(19) United States
(12) Patent Application Publication
(10) Pub. No.: US 2026/0207734 A1
(43) Pub. Date: Jul. 23, 2026

(54) CORONAVIRUS VACCINES AND METHODS OF USE

(71) Applicant: BioNTech SE, Mainz (DE)

(72) Inventors: Richard B. Gaynor, Carmel, IN (US); Lakshmi Srinivasan, Lexington, MA (US); Asaf Poran, Stoneham, MA (US); Dewi Harjanto, Brookline, MA (US); Christina Murphy Arieta, Lexington, MA (US); Daniel Abram Rothenberg, Melrose, MA (US); John Srouji, Medford, MA (US); Stefanie Krumm, Partenheim (DE); Kaushik Thanki, Wiesbaden (DE); Annette Vogel, Mainz (DE); Ugur Sahin, Mainz (DE); Yushu Joy Xie, Cambridge, MA (US)

(21) Appl. No.: 18/694,596

(22) PCT Filed: Sep. 22, 2022
(86) PCT No.: PCT/US2022/044400

§ 371 Date: Mar. 22, 2024

Related U.S. Application Data
(60) Provisional application No. 63/246,902, filed on Sep. 22, 2021.
(60) Provisional application No. 63/320,187, filed on Mar. 15, 2022.

(51) Int. Cl. A61K 39/215; A61K 9/127; A61K 39/00; A61P 37/04

(52) U.S. Cl. CPC A61K 39/215; A61K 9/127; A61P 37/04; A61K 2039/53

(57) ABSTRACT

Compositions and methods for the prevention and/or treatment of a viral infection, in particular of the Coronaviridae family.

### CROSS-REFERENCE TO RELATED APPLICATIONS

This application claims the benefit of U.S. Provisional Application No. U.S. 63/246,902 filed Sep. 22, 2021, and U.S. Provisional Application No. 63/320,187 filed Mar. 15, 2022, the content of each of which is hereby incorporated herein in its entirety.

### BACKGROUND

Newly emerging acute respiratory virus infections caused by novel coronavirus is a significant public health concern. Importantly, there are no vaccines or specific antivirals at the time of an outbreak, specifically, for example the MERS-CoV of 2015, or 2019 SARS CoV-2 infections. The 2019 SARS COV-2 infection outbreak in December of 2019 claimed more than 2000 lives in less than 2 months from the first reported case. Accordingly, novel and easily scalable therapeutics are necessary to combat a disease caused by such a viral infection.

The present application contains a Sequence Listing which has been submitted electronically in XML format and is hereby incorporated by reference in its entirety. Said XML file copy, created on Sep. 15, 2025, is named 2013237-0996.xml and is 23,117,867 bytes in size.

### SUMMARY

Patients who are immunocompromised because of autoimmunity, organ transplant or immunosuppressive treatment have reduced ability to produce antibody responses to vaccinations (Rousseau et al., A H1N1v 2009 vaccine in cancer patients treated with cytotoxic chemotherapy and/or targeted therapy: the VACANCE study. Ann Oncol. 2012 February;23 (2): 450-7). Seropositivity after vaccination is decreased in immunocompromised patients (Haidar et al., Immunogenicity of COVID-19 Vaccination in Immunocompromised Patients: An Observational, Prospective Cohort Study Interim Analysis. medRxiv 2021.06.28.21259576). While immunocompromised patients were excluded from the original clinical trials testing current SARS-CoV-2 vaccines targeting spike protein, follow-up analysis of immunocompromised patients have shown reduced capacity for patients to mount broad and durable anti-spike antibody responses, particularly in older patient cohorts (Boyarsky, et al., Immunogenicity of a Single Dose of SARS-CoV-2 Messenger RNA Vaccine in Solid Organ Transplant Recipients. JAMA. 2021; 325 (17): 1784-1786; Rincon-Arevalo, et al., Impaired antigen-specific memory B cell and plasma cell responses including lack of specific IgG upon SARS-CoV-2 BNT162b2 vaccination among Kidney Transplant and Dialysis patients. medRxiv 2021.04.15.21255550). Thus, induction of T cell responses to other SARS-CoV-2 viral proteins may be beneficial to enhance both cellular and humoral immunity. Thus, there is a need for a SARS-CoV-2 vaccine that specifically targets T cell responses. Patients undergoing anti-CD20 treatment were shown to be able to develop functional T cells with vaccination (Apostolidis et al., Altered cellular and humoral immune responses following SARS-CoV-2 mRNA vaccination in patients with multiple sclerosis on anti-CD20 therapy. Nature Medicine, vol. 27, pp. 1990-2001 (2021)), indicating that patients with impaired B cells may still develop SARS-CoV 2-specific T cells in response to vaccination.

Provided herein is an immunogenic composition (e.g., a vaccine) that specifically targets one or more T cell responses to a pathogen (e.g., in some embodiments a virus), including CD4+ T cell responses and/or CD8+ T cell responses, and/or leverages long term persistence of T cell immunity. In some embodiments, an immunogenic composition (e.g., vaccine) provided herein can specifically target one or more T cell responses to one or more polypeptide antigens of a pathogen (e.g., in some embodiments one or more viral antigens), including, e.g., non-structural proteins, nucleocapsid, membrane protein and/or envelope protein of one or more viruses.

In one aspect, provided herein is a SARS-CoV-2 immunogenic composition (e.g., a vaccine) that specifically targets one or more T cell responses, including CD4+ T cell responses and/or CD8+ T cell responses, and/or leverages long term persistence of T cell immunity. In some embodiments, a SARS-CoV-2 vaccine provided herein can specifically target one or more T cell responses to a polypeptide antigen of SARS-CoV-2, including, e.g., nucleocapsid, membrane protein and/or envelope protein of SARS-CoV-2. SARS-CoV-2 immunogenic compositions (e.g., a vaccines) provided herein can be useful for eliciting one or more T cell responses to SARS-CoV-2 in all patients. Protection from COVID19 has been observed in patients deficient in humoral immunity when T cell responses were present (Bange, et al., CD8+ T cells contribute to survival in patients with COVID-19 and hematologic cancer. Nat Med 27, 1280-1289 (2021)) and the longevity of T cell responses from related infections from the original SARS-CoV epidemic (Le Bert et al., SARS-CoV-2-specific T cell immunity in cases of COVID-19 and SARS, and uninfected controls. Nature 584, 457-462 (2020)). In some embodiments, the present disclosure, among other things, provides a particular insight that SARS-CoV-2 immunogenic compositions (e.g., a vaccines) provided herein can be particularly useful for eliciting one or more T cell responses to SARS-CoV-2 in patients that have been immunocompromised in their humoral immunity, such as, for example, in some embodiments through cancer (e.g., B cell lymphoma), treatment with rituximab, methotrexate or other immunosuppressive treatment targeting the humoral immune response, or patients undergoing organ transplant. In some embodiments, T cell responses induced by SARS-CoV-2 immunogenic compositions (e.g., a vaccines) described herein can protect patients from severe COVID-19 and provide long lasting protection through T cell immunity to the SARS-CoV-2 immunogenic composition (e.g., a vaccine) provided herein. Additionally or alternatively, in some embodiments, SARS-CoV-2 immunogenic compositions (e.g., a vaccines) provided herein can be used to overcome SARS-CoV-2 variants that could reduce efficacy of other vaccines, such as those that do not target T cell responses (Davis et al., Reduced neutralisation of the Delta (B.1.617.2) SARS-CoV-2 variant of concern following vaccination. PLoS Pathog., 17 (12): e1010022 (2021); Tada et al., Comparison of Neutralizing Antibody Titers Elicited by mRNA and Adenoviral Vector Vaccine against SARS-CoV-2 Variants. bioRxiv 2021.07.19.452771). In some embodiments, the immunogenic compositions described herein are used to treat a subject with SARS-CoV-2B1.1.529 (omicron) variant or to immunize a subject against SARS-CoV-2 B1.1.529 (omicron) variant. In some embodiments, SARS-CoV-2 immunogenic compositions (e.g., a vaccines) provided herein can be used to complement and/or enhance other immunogenic compositions (e.g., a vaccines), such as those that do not target T cell responses. For example, in some embodiments, SARS-CoV-2 immunogenic compositions (e.g., a vaccines) provided herein can be used to enhance B cell responses through increased CD4+ T cell activation.

Coronaviruses are single positive stranded RNA viruses that have emerged occasionally from zoonotic sources to infect human populations. Most of the infections in humans cause mild respiratory symptoms, though some recent coronavirus infections in the last decade have resulted in severe morbidity and mortality. These include the severe acute respiratory syndrome coronavirus (SARS-CoV), middle east respiratory syndrome coronavirus (MERS-CoV) and the currently ongoing pandemic of SARS-CoV-2. Infection with these viruses can lead to acute respiratory distress resulting in a high mortality rate. SARS-CoV originated in 2002 in South China and its global spread led to 8096 cases and 774 deaths. The first case of MERS-CoV emerged in 2012 in Saudi Arabia and since then a total of 2494 cases and 858 associated deaths have been reported. 2019 SARS CoV-2 emerged in Wuhan, China at the end of December 2019 and by Mar. 8, 2020 had resulted in 118,096 cases including 4262 deaths globally. The rapid spread of 2019 SARS-CoV-2 resulted in the World Health Organization declaring a global pandemic of international concern.

All three coronaviruses SARS-CoV, MERS-CoV and the recently emergent SARS CoV-2 belong to the genus beta coronaviridae. SARS CoV-2 has a genome size of 30 kilobases that encodes for at least four (4) structural (spike [S], envelope [E], membrane [M], and nucleocapsid [N]) and at least sixteen (16) non-structural (NSP 1-16) proteins. S protein facilitates viral entry into target cells and entry depends on binding of the spike protein to a cellular receptor ACE2 for both SARS-CoV and SARS-CoV-2. Both viruses share a 76% amino acid identity across the genome.

The field of the present disclosure relates to immunotherapeutic peptides, nucleic acids encoding the peptides, peptide binding agents, and their use, for example, in the immunotherapy of a viral disease. In one aspect, the present disclosure provides viral epitopes expressed in virus infected cells, useful alone or in combination with other anti-viral, or immunomodulatory agents to treat viral infection. The present disclosure is useful in immunotherapy for a coronavirus infection.

Provided herein is a method of treating or preventing an infection by a virus (e.g., SARS-CoV-2) or treating a respiratory disease or condition associated with an infection by a virus (e.g., SARS-CoV-2) comprising administering to a subject with a B cell immunodeficiency a pharmaceutical composition comprising: (i) a polypeptide comprising at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (ii) a polynucleotide encoding a polypeptide, wherein the polypeptide comprises at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (iii) a T cell receptor (TCR) or a T cell comprising the TCR, wherein the TCR binds to an epitope sequence of the polypeptide in complex with a corresponding HLA class I or class II molecule; (iv) an antigen presenting cell comprising (i) or (ii); or (v) an antibody or B cell comprising the antibody, wherein the antibody binds to an epitope sequence of the polypeptide.

In some embodiments, the subject has a reduced ability to produce an antibody response to an antigen compared to a subject without a B cell immunodeficiency.

In some embodiments, the subject has a reduced ability to produce an antibody response to a vaccination compared to a subject without a B cell immunodeficiency.

In some embodiments, the subject has a reduced ability to produce an anti-spike protein antibody response and/or an anti-RBD antibody response compared to a subject without a B cell immunodeficiency.

In some embodiments, the subject can produce a T cell response or does not have a reduced ability to produce a T cell response compared to a subject without a B cell immunodeficiency.

In some embodiments, the pharmaceutical composition is protective against a variant of 2019 SARS CoV-2.

In some embodiments, the variant of 2019 SARS CoV-2 is alpha, beta, gamma, delta, epsilon, zeta, eta, theta, iota, kappa or lambda.

In some embodiments, the subject produces a T cell response to an epitope of the polypeptide.

In some embodiments, the subject produces a T cell response to the epitope sequence from ORF1ab, the epitope sequence from membrane glycoprotein (M) and/or the epitope sequence from nucleocapsid phosphoprotein (N).

In some embodiments, the subject is an organ transplant recipient.

In some embodiments, the organ transplant recipient is a sold organ transplant recipient, a stem cell transplant recipient or a bone marrow transplant recipient.

In some embodiments, the subject received an organ transplant less than 1 year, less than 6 months or less than 3 months after the pharmaceutical composition is administered.

In some embodiments, the subject is expected to receive an organ transplant less than 1 year, less than 6 months or less than 3 months prior to the pharmaceutical composition being administered.

In some embodiments, the subject has a cancer.

In some embodiments, the cancer is a B cell cancer.

In some embodiments, the B cell cancer is a B cell lymphoma or a B cell leukemia.

In some embodiments, the subject has an autoimmune disease or condition.

In some embodiments, the autoimmune disease or condition is Addison disease, Anti-NMDA receptor encephalitis, antisynthetase syndrome, Aplastic anemia, autoimmune anemias, Autoimmune hemolytic anemia, Autoimmune pancreatitis, Behcet's Disease, bullous skin disorders, Celiac disease-sprue, chronic fatigue syndrome, Chronic inflammatory demyelinating polyneuropathy, chronic lymphocytic leukemia, Crohn's disease, Dermatomyositis, Devic's disease, Erythroblastopenia, Evans syndrome, Focal segmental glomerulosclerosis, Granulomatosis with polyangiitis, Graves disease, Graves' ophthalmopathy, Guillain-Barre syndrome, Hashimoto thyroiditis, idiopathic thrombocytopenia purpura (ITP), IgA nephropathy, IgA-mediated autoimmune diseases, IgG4-related disease, Inflammatory bowel disease, Juvenile idiopathic arthritis, Multiple sclerosis, Myasthenia gravis, myeloma, non-Hodgkin's lymphoma, Opsoclonus myoclonus syndrome (OMS), Pemphigoid, Pemphigus, pemphigus vulgaris, Pernicious anemia, polymyositis, Psoriasis, pure red cell aplasia, Reactive arthritis, Rheumatoid arthritis, Sarcoidosis, scleroderma, Sjögren syndrome, Systemic lupus erythematosus, Thrombocytopenia purpura, Thrombotic thrombocytopenia purpura, Type I diabetes, Ulcerative colitis, Vasculitis and Vitiligo.

In some embodiments, the subject does not have congenital agammaglobulinemia or congenital IgA deficiency.

In some embodiments, the subject does not have HIV or AIDS.

In some embodiments, the subject is receiving an immunosuppressive agent or has received an immunosuppressive agent less than 1 year, less than 6 months or less than 3 months prior to the administering of the pharmaceutical composition.

In some embodiments, the immunosuppressive agent is abatacept, abrilumab, acalabrutinib, adalimumab, adrenocorticotropic hormone, agatolimod sodium, aldesleukin, alefacept, alemtuzumab, alisertib, alvespimycin hydrochloride, alvocidib, ambrisentan, aminocamptothecin, amiselimod, anakinra, andecaliximab, andrographolides, anifrolumab, antithymocyte Ig, apatinib, apelisib, asparaginase, atacicept, atezolizumab, avelumab, azacitidine, azathioprine, bafetinib, baminercept, baricitinib, basiliximab, becatecarin, begelomab, belatacept, belimumab, bemcentinib, bendamustine, bendamustine, betalutin with lilotomab, bevacizumab, BIIB033, BIIB059, BIIB061, bimekizumab, binimetinib, bleomycin, blinatumomab, bortezomib, brentuximab vedotin, bryostatin 1, bucillamine, buparlisib, busulfan, canakinumab, capecitabine, carboplatin, carfilzomib, carmustine, cediranib maleate, cemiplimab, ceralifimod, cerdulatinib, certolizumab, cetuximab, chidamide, chlorambucil, cilengitide, cirmtuzumab, cisplatin, cladribine, clazakizumab, clemastine, clioquinol, corticosteroids, cyclophosphamide, cyclosporine, cytarabine, cytotoxic chemotherapy, daclizumab, dalfampridine, daprolizumab pegol, daratumumab, dasatinib, defactinib, defibrotide, denosumab, dexamethasone, diacerein, dimethyl fumarate, dinaciclib, diroximel fumarate, doxorubicin, doxorubicin, durvalumab, duvelisib, duvortuxizumab, eculizumab, efalizumab, eftilagimod alpha, a neuropeptide combination of metenkefalin and tridecactide, elezanumab, elotuzumab, encorafenib, enfuvirtida, entinostat, entospletinib, enzastaurin, epacadostat, epirubicin, epratuzumab, eritoran tetrasodium, etanercept, etoposide, etrolizumab, everolimus, evobrutinib, filgotinib, fingolimod, firategrast, fludarabine, fluorouracil, fontolizumab, forodesine hydrochloride, fostamatinib, galunisertib, ganetespib, ganitumab, gemcitabine, gemtuzumab ozogamicin, gerilimzumab, glasdegib, glassia, glatiramer acetate, glembatumumab vedotin, glesatinib, golimumab, guadecitabine, hydrocortisone, hydroxychloroquine sulfate, hydroxyurea, ibritumomab tiuxetan, ibrutinib, ibudilast, idarubicin, idebenone, idelalisib, ifosfamide, iguratimod, imatinib, imexon, infliximab, inotuzumab ozogamicin, interferon alfa-2, interferon beta-la, interferon beta-1b, interferon gamma-1, ipilimumab, irofulven, isatuximab, ispinesib, itacitinib, ixazomib, lapatinib, laquinimod, laromustine, ld-aminopterin, leflunomide, lenalidomide, lenvatinib, letrozole, levamisole, levocabastine, lipoic acid, lirilumab, lonafarnib, lumiliximab, maraviroc, masitinib, mavrilimumab, melphalan, mercaptopurine, methotrexate, methoxsalen, methylprednisone, milatuzumab, mitoxantrone, mizoribine, mocetinostat, monalizumab, mosunetuzumab, motesanib diphosphate, moxetumomab pasudotox, muromonab-CD3, mycophenolate mofetil, mycophenolic acid, namilumab, natalizumab, navitoclax, neihulizumab, nerispirdine, neurovax, niraparib, nivolumab, obatoclax mesylate, obinutuzumab, oblimersen sodium, ocrelizumab, ofatumumab, olokizumab, opicinumab, oprelvekin, osimertinib, otelixizumab, oxaliplatin, oxcarbazepine, ozanimod, paclitaxel, pacritinib, palifermin, panobinostat, pazopanib, peficitinib, pegfilgrastim, peginterferon beta-la, pegsunercept (peg stnf-ri), pembrolizumab, pemetrexed, penclomedine, pentostatin, perifosine, pevonedistat, pexidartinib, picoplatin, pidilizumab, pivanex, pixantrone, pleneva, plovamer acetate, polatuzumab vedotin, pomalidomide, ponatinib, ponesimod, prednisone/prednisolone, pyroxamide, ravulizimab-cwvz, recombinant il-12, relatlimab, rhigf-1, rhigm22, rigosertib, rilonacept, ritonavir, rituximab, ruxolitinib, sarilumab, secukinumab, selumetinib, simvastatin, sintilimab, siplizumab, siponimod, sirolimus (rapamycin), sirukumab, sitravatinib, sonidegib, sorafenib, sotrastaurin acetate, sunitinib, sunphenon epigallocatechin-gallate, tabalumab, tacrolimus, talabostat mesylate, talacotuzumab, tanespimycin, tegafur/gimeracil/oteracil, temozolomide, temsirolimus, tenalisib, terameprocol, teriflunomide, thalidomide, thiarabine, thiotepa, tipifarnib, tirabrutinib, tislelizumab, tivozanib, tocilizumab, tofacitinib, tregalizumab, tremelimumab, treosulfan, ublituximab, umbralisib, upadacitinib, urelumab, ustekinumab, varlilumab, vatelizumab, vedolizumab, veliparib, veltuzumab, venetoclax, vinblastine, vincristine, vinorelbine ditartrate, visilizumab, vismodegib, vistusertib, voriconazole, vorinostat, vosaroxin, ziv-aflibercept or any combination thereof.

In some embodiments, the immunosuppressive agent is A2aR antagonist, Akt inhibitor, anti CD20, Anti-amyloidotic (AA) Agent, anti-CD37 protein therapeutic, anti-CTLA4 mAb, Anti-CXCR4, anti-huCD40 mAb, anti-LAG3 mAb, anti-PD-1 mAb, anti-PD-L1 agent, anti-PD-L1 agent, anti-PD-L1 mAb, anti-TGFb mAb, anti-TIGIT mAb, anti-TIM-3 mAb, Aurora kinase inhibitor, Bcl-2 Inhibitor, bifunctional fusion protein targeting TGFb and PD-L1, bispecific anti-PD-1 and anti-LAG3 mAb, CD1d ligand, CD40 agonist, Complement C5a inhibitor, CSF1R inhibitor, EZH2 inhibitor, FGFR3 inhibitor, FGFR4 inhibitor, FGFrR3 inhibitor, glucocorticoid-induced tumor necrosis factor receptor-related gene agonist, glutaminase inhibitor, Human monoclonal antibody against IL-12, ICOS agonist, IDO1 inhibitor, IL2 mutein, IL2 receptor agonist, MEK inhibitor, multitargeted receptor tyrosine kinase inhibitor, neutrophil elastase inhibitor, Notch Inhibitor, p38 MAPK inhibitor, PD-1 inhibitor, recombinant human Flt3L, ROCK inhibitor, selective sphingosine-1-phosphate receptor modulator, Src kinase inhibitor, TLR4 agonist, TLR9 agonist, or any combination thereof.

In some embodiments, the subject is greater than 55, 56, 57, 58, 59, 60, 65, 70, 75 or 80 years of age.

In some embodiments, the polypeptide comprises (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N).

In some embodiments, the sequence comprising an epitope sequence from ORF1ab is C-terminal to the sequence comprising an epitope sequence from nucleocapsid phosphoprotein (N).

In some embodiments, the sequence comprising an epitope sequence from ORF1ab is N-terminal to the sequence comprising an epitope sequence from membrane glycoprotein (M).

In some embodiments, the sequence comprising an epitope sequence from nucleocapsid phosphoprotein (N) is N-terminal to the sequence comprising an epitope sequence from membrane glycoprotein (M).

In some embodiments, the polypeptide comprises (a) 2, 3, 4, 5, 6, 7, 8, 9 or 10 or more epitope sequences from ORF1ab, (b) a sequence comprising an epitope sequence from membrane glycoprotein (M) and (c) a sequence comprising an epitope sequence from nucleocapsid phosphoprotein (N).

In some embodiments, the epitope sequence from ORF1ab is an epitope sequence from a non-structural protein (NSP).

In some embodiments, the non-structural protein (NSP) is selected from the group consisting of NSP1, NSP2, NSP3, NSP4 and combinations thereof.

In some embodiments, the polypeptide comprises a sequence comprising an epitope sequence from NSP1, a sequence comprising an epitope sequence from NSP2, a sequence comprising an epitope sequence from NSP3 and a sequence comprising an epitope sequence from NSP4.

In some embodiments, the epitope sequence from ORF1ab is selected from the group consisting of YLFDESGEFKL, YLFDESGEF, FGDDTVIEV, QLMCQPILL, TTDPSFLGRY, PTDNYITTY, PSFLGRY, AEAELAKNV, KTIQPRVEK and any combination thereof.

In some embodiments, the epitope sequence from nucleocapsid glycoprotein (N) is LLLDRLNQL.

In some embodiments, the epitope sequence from membrane phosphoprotein (M) is VATSRTLSY.

In some embodiments, the polypeptide comprises an epitope sequence from nucleocapsid glycoprotein (N) that is LLLDRLNQL and an epitope sequence from membrane phosphoprotein (M) that is VATSRTLSY.

In some embodiments, the polypeptide comprises (a) each of the following epitope sequences from ORF1ab: YLFDESGEFKL, YLFDESGEF, FGDDTVIEV, QLMCQPILL, TTDPSFLGRY, PTDNYITTY, PSFLGRY, AEAELAKNV, KTIQPRVEK; (b) an epitope sequence from nucleocapsid glycoprotein (N) that is LLLDRLNQL; and (c) an epitope sequence from membrane phosphoprotein (M) that is VATSRTLSY.

In some embodiments, the sequence comprising an epitope sequence from ORF1ab is selected from the group consisting of the following sequences or fragments thereof:

MVTNNTFTLKVPHVGEIPVAYRKVLLKTIQPRVEKYLFDESGEFKLSEVG
PEHSLAEYYIFFASFYY; MVTNNTFTLKVPHVGEIPVAYRKVLLKTIQP
RVEKYLFDESGEFKLSEVGPEHSLAEY; APKEIIFLEGETLFGDDTVIE
VAIILASFSAST; APKEIIFLEGETLFGDDTVIEV; HTTDPSFLGRYM
SALFADDLNQLTGYHTDFSSEIIGYQLMCQPILLAEAELAKNVSLILGTV
SWNL; TTDPSFLGRYMSALFADDLNQLTGYHTDFSSEIIGYQLMCQPIL
LAEAELAKNVSLILGTVSWNL; LLSAGIFGAITDVFYKENSYKVPTDNY
ITTY; and combinations thereof.

In some embodiments, the sequence comprising an epitope sequence from membrane glycoprotein (M) is selected from the group consisting of the following sequences or fragments thereof:

ADSNGTITVEELKKLLEQWNLVIGFLFLTWICLLQFAYANRNRFLYIIKL
IFLWLLWPVTLACFVLAAVYRINWITGGIAIAMACLVGLMWLSYFIASFR
LFARTRSMWSFNPETNILLNVPLHGTILTRPLLESELVIGAVILRGHLRI
AGHHLGRCDIKDLPKEITVATSRTLSYYKLGASQRVAGDSGFAAYSRYRI
GNYKLNTDHSSSSDNIALLVQ; FAYANRNRFLYIIKLIFLWLLWPVTLA
CFVLAAVYRINWITGGIAIAMACLVGLMWLSYFIASFRLF; LGRCDIKD
LPKEITVATSRTLSYYKLGASQRVA; KLLEQWNLVIGF; NRNRFLYII
KLIFLWLLWPVTLACFVLAAVY; SELVIGAVILRGHLRIAGHHLGR;
VATSRTLSYYKLGASQRV; GLMWLSYF; and combinations
thereof.

In some embodiments, the sequence comprising an epitope sequence from nucleocapsid phosphoprotein (N) is selected from the group consisting of the following sequences or fragments thereof:

KDLSPRWYFYYLGTGPEAGLPYGANKDGIIWVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQA; RMAGNGGDAALALLLLDRLNQLESKMSGKG
QQQ; YKHWPQIAQFAPSASAFFGMSRIGMEVTPSGTWLTYTGAIKLDDK
DPNFKDQVILLNKHIDAYKTFP; SPARMAGNGGDAALALLLLDRLNQLE
SKMSGKGQQQQGQTVTKKSAAEASKKPRQKRTATKAYNVTQAFGRRGPEQ
TQGNFGDQELIRQGTDYKHWPQIAQFAPSASAFFGMSRIGMEVTPSGTWL
TYTGAIKLDDKDPNFKDQVILLNKHIDAYKTFPPTEPKKDK and
combinations thereof.

In some embodiments, the polypeptide comprises one or more linker sequences.

In some embodiments, the one or more linker sequences are selected from the group consisting of GGSGGGGSGG, GGSLGGGGSG.

In some embodiments, the one or more linker sequences comprise cleavage sequences.

In some embodiments, the one or more cleavage sequences are selected from the group consisting of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA.

In some embodiments, the polypeptide comprises a transmembrane domain sequence.

In some embodiments, the transmembrane domain sequence is C-terminal to the sequence comprising an epitope sequence from ORF1ab, the sequence comprising an epitope sequence from membrane glycoprotein (M) and the sequence comprising an epitope sequence from nucleocapsid phosphoprotein (N).

In some embodiments, the transmembrane domain sequence is EQYIKWPWYIWLGFIAGLIAIVMVTIMLCCMTSCCSCLKGCCSCGSCCKFDEDDSEPVLKGVKL HYT.

In some embodiments, the polypeptide comprises an SEC sequence.

In some embodiments, the SEC sequence is N-terminal to the sequence comprising an epitope sequence from ORF1ab, the sequence comprising an epitope sequence from membrane glycoprotein (M) and the sequence comprising an epitope sequence from nucleocapsid phosphoprotein (N).

In some embodiments, the SEC sequence is MFVFLVLLPLVSSQCVNLT.

In some embodiments, the composition comprises the polynucleotide encoding the polypeptide.

In some embodiments, the polynucleotide is an mRNA.

In some embodiments, the polynucleotide comprises a codon optimized sequence for expression in a human.

In some embodiments, the polynucleotide comprises a dEarI-hAg sequence.

In some embodiments, the dEarI-hAg sequence is ATTCTTCTGGTCCCCACAGACTCAGAGAGAACCC, optionally wherein each T is a U.

In some embodiments, the polynucleotide comprises a Kozak sequence.

In some embodiments, the Kozak sequence is GCCACC.

In some embodiments, the polynucleotide comprises an F element sequence.

In some embodiments, the F element sequence is a 3 UTR of amino-terminal enhancer of split (AES).

In some embodiments, the F element sequence is CTGGTACTGCATGCACGCAATGCTAGCTGCCCCTTTCCCGTCCTGGGTACCCCGAGTCTCCC CCGACCTCGGGTCCCAGGTATGCTCCCACCTCCACCTGCCCCACTCACCACCTCTGCTAGTTC CAGACACCTCC, optionally wherein each T is a U.

In some embodiments, the polynucleotide comprises an I element sequence.

In some embodiments, the I element sequence is a 3′ UTR of mitochondrially encoded 12S rRNA (mtRNR1).

In some embodiments, the I element sequence is CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA ATTTCGTGCCAGCCACACC, optionally wherein each T is a U.

In some embodiments, the polynucleotide comprises a poly A sequence.

In some embodiments, the poly A sequence is AAAAAAAAAAAAAAAAAAAAAAAAAAAAAAGCATATGACTAAAAAAAAAAAAAAAAAAA AAAAAAAAAAAAAAAAAAAAAAAAAAAAAAAAAAAAAAAAAAAAAAAAAAA, optionally wherein each T is a U.

In some embodiments, each of the epitope sequences from the ORF1ab, the membrane glycoprotein, and the nucleocapsid phosphoprotein are from 2019 SARS-CoV-2.

In some embodiments, one or more or each epitope elicits a T cell response.

In some embodiments, one or more or each epitope has been observed by mass spectrometry as being presented by an HLA molecule.

In some embodiments, the composition comprises (i) a polypeptide with at least 70%, at least 75%, at least 80%, at least 85%, 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; (ii) a polynucleotide encoding a polypeptide with at least 70%, at least 75%, at least 80%, at least 85%, 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or (iii) a polynucleotide with at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS Con2, RS C7n2, RS C7n4, RS C7n4full, RS C8n2, RS C5n2full, RS Con2full, RS C7n2full and RS C8n2full.

In some embodiments, the composition comprises (i) a polypeptide with at least 70%, at least 75%, at least 80%, at least 85%, at least 90%, at least 95%, or 100% sequence identity to a sequence selected from the group consisting of RS C7p1, RS C7p2, RS C7p2full, RS C7p4 and RS C7p4full; or (ii) a polynucleotide with at least 70%, at least 75%, at least 80%, at least 85%, at least 90%, at least 95%, or 100% sequence identity to a sequence selected from the group consisting of SEQ ID NOs: RS C7n1, RS C7n2, RS C7n2full, RS C7n4 and RS C7n4full.

In some embodiments, the pharmaceutical composition comprises a pharmaceutically acceptable excipient, carrier, or diluent.

Provided herein is a method of treating or preventing an infection by a virus or treating a respiratory disease or condition associated with an infection by a virus comprising administering to a subject with a B cell immunodeficiency a pharmaceutical composition comprising: (i) a polypeptide comprising an epitope sequence of Table 1A, Table 1B, Table 1C, Table 2Ai, Table 2Aii, Table 2B and/or Table 16; (ii) a polynucleotide encoding the polypeptide comprising an epitope sequence of Table 1A, Table 1B, Table 1C, Table 2Ai, Table 2Aii, Table 2B and/or Table 16; (iii) a T cell receptor (TCR) or a T cell comprising the TCR, wherein the TCR binds to the epitope sequence in complex with a corresponding HLA class I or class II molecule; (iv) an antigen presenting cell comprising (i) or (ii); or (v) an antibody or B cell comprising the antibody, wherein the antibody binds to the epitope sequence.

In some embodiments, the epitope sequence comprises one or more or each of the following: YLFDESGEFKL, YLFDESGEF, FGDDTVIEV, LLLDRLNQL, QLMCQPILL, TTDPSFLGRY, PTDNYITTY, PSFLGRY, AEAELAKNV, VATSRTLSY and KTIQPRVEK.

In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV, FGADPIHSL, NYNYLYRLF, KYIKWPWYI, KWPWYIWLGF, LPFNDGVYF, QPTESIVRF, IPFAMQMAY, YLQPRTFLL and RLQSLQTYV.

In some embodiments, the epitope sequence is from an orflab protein.

In some embodiments, the epitope sequence is from an orfla protein

In some embodiments, the epitope sequence is from a surface glycoprotein(S) or a shifted reading frame thereof.

In some embodiments, the epitope sequence is from a nucleocapsid phosphoprotein (N).

In some embodiments, the epitope sequence is from an ORF3a protein.

In some embodiments, the epitope sequence is from a membrane glycoprotein (M).

In some embodiments, the epitope sequence is from an ORF7a protein.

In some embodiments, the epitope sequence is from an ORF8 protein.

In some embodiments, the epitope sequence is from an envelope protein (E).

In some embodiments, the epitope sequence is from an ORF6 protein.

In some embodiments, the epitope sequence is from an ORF7b protein.

In some embodiments, the epitope sequence is from an ORF10 protein.

In some embodiments, the epitope sequence is from an ORF9b protein.

Provided herein is a method of treating or preventing an infection by a virus or treating a respiratory disease or condition associated with an infection by a virus comprising administering to a subject with a B cell immunodeficiency a pharmaceutical composition comprising: a polypeptide having an amino acid sequence with at least 70%, at least 75%, at least 80%, at least 85%, at least 90%, at least 95% or 100% sequence identity to a sequence of any one of the sequences depicted in column 2 of Table 11, column 2 of Table 12 or column 3 of Table 15; or a recombinant polynucleotide encoding a polypeptide having an amino acid sequence with at least at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15.

In some embodiments, the pharmaceutical composition comprises a polypeptide with at least 70%, at least 75%, at least 80%, at least 85%, 90%, at least 95% or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or a polynucleotide encoding a polypeptide with at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full.

In some embodiments, the pharmaceutical composition comprises a polynucleotide with at least 70%, at least 75%, at least 80%, at least 85%, at least 90%, at least 95% or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS Con2, RS C7n2, RS C8n2, RS C5n2full, RS Con2full, RS C7n2full, RS C8n2full, RS C7n4, and RS C7n4full.

In some embodiments, the polynucleotide is an mRNA.

In some embodiments, the pharmaceutical composition further comprises one or more lipid components.

In some embodiments, the one or more lipid components comprise a lipid nanoparticle (LNP).

In some embodiments, the LNP encapsulates the recombinant polynucleotide construct.

In some embodiments, the polypeptide is synthetic.

In some embodiments, the polypeptide is recombinant.

In some embodiments, the polypeptide is from 8-1000 amino acids in length.

In some embodiments, the epitope sequence binds to or is predicted to bind to an HLA class I or class II molecule with a KD of 1000 nM or less.

In some embodiments, the epitope sequence binds to or is predicted to bind to an HLA class I or class II molecule with a KD of 500 nM or less.

In some embodiments, the epitope sequence comprises a sequence of a viral protein expressed by a virus-infected cell of the subject.

In some embodiments, the virus is a coronavirus.

In some embodiments, the virus is 2019 SARS-CoV 2.

In some embodiments, an HLA molecule expressed by the subject is unknown at the time of administration.

In some embodiments, the ability of the virus to avoid escape of recognition by an immune system of the subject is less compared to the ability of the virus to avoid escape of recognition by an immune system of a subject administered a pharmaceutical composition containing an epitope from a single protein or epitopes from fewer proteins than in the pharmaceutical composition administered according a method described herein.

In some embodiments, the subject expresses an HLA molecule encoded by an HLA allele of any one of Table 1A, Table 1B, Table 1C, Table 2Ai, Table 2Aii, Table 2B and Table 16 and the epitope sequence is an HLA allele-matched epitope sequence.

In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV and FGADPIHSL.

In some embodiments, the method further comprises administering to the subject an additional therapy for a 2019 SARS-CoV 2 viral infection.

In some embodiments, the method further comprises administering to the subject (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (a) or (b).

In some embodiments, the vaccine or therapeutic of (a) or (b) is administered to the subject once.

In some embodiments, the vaccine or therapeutic of (a) or (b) is administered to the subject more than once.

In some embodiments, the vaccine or therapeutic is administered at least two times, wherein the first administered dose is a priming dose, and the second and subsequent doses are booster dose(s).

In some embodiments, the priming and the booster doses are administered at an interval of at least 21 days.

In some embodiments, an interval between two booster doses is at least 30 days, at least 60 days, or at least 90 days.

In some embodiments, the vaccine or therapeutic is administered once each year.

In some embodiments, the vaccine or therapeutic is administered twice each year.

In some embodiments, the vaccine or therapeutic is administered at a high priming or loading dose for the first dose, and at a reduced boosting or maintenance dose for the subsequent doses.

In some embodiments, the subject receives a lower dose of or a lower frequency of a SARS-CoV spike vaccine than a subject receiving the SARS-CoV spike vaccine alone.

Provided herein is a method of treating or preventing an infection by a virus or treating a respiratory disease or condition associated with an infection by a virus comprising administering to a subject in need thereof a pharmaceutical composition comprising: (i) a recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a recombinant polynucleotide encoding a 2019 SARS-CoV 2 spike protein or a variant or fragment thereof; wherein the ratio (e.g., mass ratio) of (i):(ii) is greater than 20:1 or less than 1:20.

In some embodiments, the ratio (e.g., mass ratio) of (i):(ii) is greater than 20:1, 30:1, 40:1, 50:1, 60:1, 70:1, 80:1, 90:1 or 100:1

In some embodiments, the ratio (e.g., mass ratio) of (i):(ii) is less than 1:20, 1:30, 1:40, 1:50, 1:60, 1:70, 1:80, 1:90 or 1:100.

Provided herein is a method of treating or preventing an infection by a virus or treating a respiratory disease or condition associated with an infection by a virus comprising administering to a subject in need thereof: (i) a first pharmaceutical composition comprising a first recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second recombinant polynucleotide encoding a 2019 SARS-CoV 2 spike protein or a variant or fragment thereof; wherein the ratio (e.g., mass ratio) of the recombinant polynucleotide in (i) to the recombinant polynucleotide in (ii) is from 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the recombinant polynucleotide in (i) to the recombinant polynucleotide in (ii) is about 1:6. In some embodiments, the ratio (e.g., mass ratio) of the recombinant polynucleotide in (i) to the recombinant polynucleotide in (ii) is about 1:3. In some embodiments, the ratio (e.g., mass ratio) of the recombinant polynucleotide in (i) to the recombinant polynucleotide in (ii) is about 1:2.

In some embodiments, the recombinant polynucleotide in (i) is present in a pharmaceutical composition at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the recombinant polynucleotide in (i) is present in a pharmaceutical composition at a dose of from 0.05 microgram to 10 micrograms, or 0.1 microgram to 5 micrograms, or 0.3 microgram to 5 micrograms. In some embodiments, the recombinant polynucleotide in (i) is present in a pharmaceutical composition at a dose of from 0.1 microgram to 20 micrograms or from 0.5 microgram to 15 micrograms.

In some embodiments, the recombinant polynucleotide in (ii) is present in a pharmaceutical composition at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the recombinant polynucleotide in (i) is present in a pharmaceutical composition at a dose of about 5 micrograms and the recombinant polynucleotide in (ii) is present in a pharmaceutical composition at a dose of about 30 micrograms. In some embodiments, the recombinant polynucleotide in (i) is present in a pharmaceutical composition at a dose of about 10 micrograms and the recombinant polynucleotide in (ii) is present in a pharmaceutical composition at a dose of about 30 micrograms. In some embodiments, the recombinant polynucleotide in (i) is present in a pharmaceutical composition at a dose of about 15 micrograms and the recombinant polynucleotide in (ii) is present in a pharmaceutical composition at a dose of about 30 micrograms.

In some embodiments, the recombinant polynucleotide in (ii) encompasses at least two separate recombinant polynucleotides, each encoding a SARS-CoV-2 S protein of a different strain or variant thereof, or an immunogenic variant or fragment thereof (e.g., in some embodiments RBD). For example, in some embodiments, the recombinant polynucleotide in (ii) encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a SARS-CoV-2 variant strain that is prevalent or rapidly spreading at the time of administration. For example, in some embodiments, the recombinant polynucleotide in (ii) encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a variant strain having one or more mutations that are characteristics of a SARS-CoV-2 variant (e.g., in some embodiments an Omicron variant such as, e.g., a Omicron BA.1, BA.2, BA.4 or BA.5 variant), or an immunogenic variant or fragment thereof. In some embodiments, at least two recombinant polynucleotides, each encoding a SARS-CoV-2 S protein of a different strain or variant thereof, or an immunogenic variant or fragment thereof, can be present in a pharmaceutical composition at a ratio (e.g., mass ratio) of 3:1 to 1:3, or 2:1 to 1:2 or 1:1.

In some embodiments, a pharmaceutical composition described herein may further comprise (iii) a recombinant polynucleotide encoding a peptide or polypeptide antigen from a pathogen associated with a non-SARS-CoV-2 respiratory disease. In some embodiments, such a non-SARS-CoV-2 respiratory disease may be flu (influenza), and/or respiratory syncytial virus.

Provided herein is a method of treating or preventing an infection by a virus or treating a respiratory disease or condition associated with an infection by a virus comprising administering to a subject in need thereof a pharmaceutical composition comprising a nanoparticle, wherein the nanoparticle comprises: (i) a first recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second recombinant polynucleotide encoding a 2019 SARS-CoV 2 spike protein or a variant or fragment thereof. In some embodiments, a first recombinant polynucleotide encodes a polypeptide comprising all of the following: a sequence comprising an epitope sequence from ORF1ab, a sequence comprising an epitope sequence from membrane glycoprotein (M), and a sequence comprising an epitope sequence from nucleocapsid phosphoprotein (N).

In some embodiments, the nanoparticle is present in the pharmaceutical composition at a dose of from 100 ng to 500 micrograms.

In some embodiments, the nanoparticle is present in the pharmaceutical composition at a dose of from 1 microgram to 100 micrograms.

In some embodiments, the nanoparticle is present in the pharmaceutical composition at a dose of from 1 microgram to 30 micrograms, 5 micrograms to 40 micrograms or 10 microgram to 50 micrograms.

In some embodiments, the nanoparticle is present in the pharmaceutical composition at a dose of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the ratio (e.g., mass ratio) of the first recombinant polynucleotide to the second recombinant polynucleotide is from about 1:50 to 50:1.

In some embodiments, the ratio (e.g., mass ratio) of the first recombinant polynucleotide to the second recombinant polynucleotide is from about 1:25 to 25:1.

In some embodiments, the ratio (e.g., mass ratio) of the first recombinant polynucleotide to the second recombinant polynucleotide is from about 1:10 to 10:1.

In some embodiments, the ratio of the first recombinant polynucleotide to the second recombinant polynucleotide is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the first recombinant polynucleotide to the second recombinant polynucleotide is about 1:6. In some embodiments, the ratio (e.g., mass ratio) of the first recombinant polynucleotide to the second recombinant polynucleotide is about 1:3. In some embodiments, the ratio (e.g., mass ratio) of the first recombinant polynucleotide to the second recombinant polynucleotide is about 1:2.

In some embodiments, the first recombinant polynucleotide is present in a pharmaceutical composition at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the first recombinant polynucleotide is present in a pharmaceutical composition at a dose of from 0.05 microgram to 10 micrograms, or 0.1 microgram to 5 micrograms, or 0.3 microgram to 5 micrograms. In some embodiments, the first recombinant polynucleotide is present in a pharmaceutical composition at a dose of from 0.1 microgram to 20 micrograms or from 0.5 microgram to 15 micrograms.

In some embodiments, the second recombinant polynucleotide is present in a pharmaceutical composition at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the second recombinant polynucleotide encompasses at least two separate recombinant polynucleotides, each encoding a SARS-CoV-2 S protein of a different strain or variant thereof, or an immunogenic variant or fragment thereof (e.g., in some embodiments RBD). For example, in some embodiments, the second recombinant polynucleotide encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a SARS-CoV-2 variant strain that is prevalent or rapidly spreading at the time of administration. For example, in some embodiments, the second recombinant polynucleotide encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a variant strain having one or more mutations that are characteristics of a SARS-CoV-2 variant (e.g., in some embodiments an Omicron variant such as, e.g., an Omicron BA.1, BA.2, BA.4 or BA.5 variant), or an immunogenic variant or fragment thereof. In some embodiments, the two recombinant polynucleotides, each encoding a SARS-CoV-2 S protein of a different strain or variant thereof, or an immunogenic variant or fragment thereof, can be present in a pharmaceutical composition at a ratio (e.g., mass ratio) of 3:1 to 1:3, or 2:1 to 1:2 or 1:1.

In some embodiments, a pharmaceutical composition described herein may comprise a third recombinant polynucleotide encoding a peptide or polypeptide antigen from a pathogen associated with a non-SARS-CoV-2 respiratory disease. In some embodiments, such a non-SARS-CoV-2 respiratory disease may be, but not limited to flu (influenza), and/or respiratory syncytial virus.

Provided herein is a method of treating or preventing an infection by a virus or treating a respiratory disease or condition associated with an infection by a virus comprising administering to a subject in need thereof: (i) a first pharmaceutical composition comprising a first nanoparticle, wherein the first nanoparticle comprises a recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second nanoparticle, wherein the second nanoparticle comprises a recombinant polynucleotide encoding a 2019 SARS-CoV 2 spike protein or a variant or fragment thereof. In some embodiments, a first pharmaceutical composition comprising a first nanoparticle, wherein the first nanoparticle comprises a recombinant polynucleotide encoding a polypeptide comprising all of the following: a sequence comprising an epitope sequence from ORF1ab, a sequence comprising an epitope sequence from membrane glycoprotein (M), and a sequence comprising an epitope sequence from nucleocapsid phosphoprotein (N).

In some embodiments, the ratio (e.g., mass ratio) of the recombinant polynucleotide in (i) to the recombinant polynucleotide in (ii) is from about 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the recombinant polynucleotide in (i) to the recombinant polynucleotide in (ii) is about 1:6. In some embodiments, the ratio (e.g., mass ratio) of the recombinant polynucleotide in (i) to the recombinant polynucleotide in (ii) is about 1:3. In some embodiments, the ratio (e.g., mass ratio) of the recombinant polynucleotide in (i) to the recombinant polynucleotide in (ii) is about 1:2.

In some embodiments, the recombinant polynucleotide in (i) is present in a pharmaceutical composition at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the recombinant polynucleotide in (i) is present in a pharmaceutical composition at a dose of from 0.05 microgram to 10 micrograms, or 0.1 microgram to 5 micrograms, or 0.3 microgram to 5 micrograms. In some embodiments, the recombinant polynucleotide in (i) is present in a pharmaceutical composition at a dose of from 0.1 microgram to 20 micrograms or from 0.5 microgram to 15 micrograms.

In some embodiments, the recombinant polynucleotide in (ii) is present in a pharmaceutical composition at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the recombinant polynucleotide in (ii) encompasses at least two separate recombinant polynucleotides, each encoding a SARS-CoV-2 S protein of a different strain or variant thereof, or an immunogenic variant or fragment thereof (e.g., in some embodiments RBD). For example, in some embodiments, the recombinant polynucleotide in (ii) encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a SARS-CoV-2 variant strain that is prevalent or rapidly spreading at the time of administration. For example, in some embodiments, the recombinant polynucleotide in (ii) encompasses a recombinant polynucleotide encoding a SARS-CoV-2 S protein of an ancestral strain (e.g., Wuhan strain) or an immunogenic variant or fragment thereof (e.g., in some embodiments RBD) and a recombinant polynucleotide encoding a SARS-CoV-2 S protein of a variant strain having one or more mutations that are characteristics of a SARS-CoV-2 variant (e.g., in some embodiments an Omicron variant such as, e.g., a Omicron BA.1, BA.2, BA.4 or BA.5 variant), or an immunogenic variant or fragment thereof. In some embodiments, at least two recombinant polynucleotides, each encoding a SARS-CoV-2 S protein of a different strain or variant thereof, or an immunogenic variant or fragment thereof, can be present in a pharmaceutical composition at a ratio (e.g., mass ratio) of 3:1 to 1:3, or 2:1 to 1:2 or 1:1.

In some embodiments, a third pharmaceutical composition (iii) may be administered to a subject in need thereof, which comprises a third nanoparticle, wherein the third nanoparticle comprises a recombinant polynucleotide encoding a peptide or polypeptide antigen from a pathogen associated with a non-SARS-CoV-2 respiratory disease. In some embodiments, such a non-SARS-CoV-2 respiratory disease may be flu (influenza), and/or respiratory syncytial virus.

In some embodiments, the first nanoparticle is present in the first pharmaceutical composition at a dose of from about 100 ng to 500 micrograms.

In some embodiments, the first nanoparticle is present in the first pharmaceutical composition at a dose of from about 1 microgram to 100 micrograms.

In some embodiments, the first nanoparticle is present in the first pharmaceutical composition at a dose of from about 1 microgram to 30 micrograms, 5 micrograms to 40 micrograms or 10 microgram to 50 micrograms.

In some embodiments, the first nanoparticle is present in the first pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the second nanoparticle is present in the second pharmaceutical composition at a dose of from about 100 ng to 500 micrograms.

In some embodiments, the second nanoparticle is present in the second pharmaceutical composition at a dose of from about 1 microgram to 100 micrograms.

In some embodiments, the second nanoparticle is present in the second pharmaceutical composition at a dose of from about 1 microgram to 30 micrograms, 5 micrograms to 40 micrograms or 10 microgram to 50 micrograms.

In some embodiments, the second nanoparticle is present in the second pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; or (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a dose of (ii)(a) or (ii)(b) that is lower than a dose of (ii)(a) or (ii)(b) administered to a subject alone.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; or (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b).

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a number of doses of (ii)(a) or (ii)(b) that is lower than a number of doses of (ii)(a) or (ii)(b) administered to a subject alone.

In some embodiments, the subject receives a dose of (ii)(a) or (ii)(b) that is at least 1.1, 1, 1.5, 2, 3, 4, 5, 6, 7, 8, 9, 10, 20, 30, 40, 50, 60, 70, 80, 90 or 100 times lower than a dose of (ii)(a) or (ii)(b) administered to a subject alone.

In some embodiments, the subject receives 1, 2, 3, 4, 5, 6, 7, 8, 9 or 10 fewer doses of (ii)(a) or (ii)(b) than the number of doses of (ii)(a) or (ii)(b) administered to a subject alone.

In some embodiments, the pharmaceutical composition of (i) is co-formulated with the pharmaceutical composition of (ii).

In some embodiments, the pharmaceutical composition of (i) is formulated separately from the pharmaceutical composition of (ii).

In some embodiments, the pharmaceutical composition of (i) is administered separately from the pharmaceutical composition of (ii).

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a dose of (i)(a) or (i)(b) that is lower than a dose of (i)(a) or (i)(b) administered to a subject alone.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a number of doses of (i)(a) or (i)(b) that is lower than a number of doses of (i)(a) or (i)(b) administered to a subject alone.

In some embodiments, the subject receives a dose of (i)(a) or (i)(b) that is at least 1.1, 1, 1.5, 2, 3, 4, 5, 6, 7, 8, 9, 10, 20, 30, 40, 50, 60, 70, 80, 90 or 100 times lower than a dose of (i)(a) or (i)(b) administered to a subject alone.

In some embodiments, the subject receives 1, 2, 3, 4, 5, 6, 7, 8, 9 or 10 fewer doses of (i)(a) or (i)(b) than the number of doses of (i)(a) or (i)(b) administered to a subject alone.

In some embodiments, the pharmaceutical composition of (i) is co-formulated with the pharmaceutical composition of (ii).

In some embodiments, the pharmaceutical composition of (i) is formulated separately from the pharmaceutical composition of (ii).

In some embodiments, the pharmaceutical composition of (i) is administered separately from the pharmaceutical composition of (ii).

In some embodiments, the pharmaceutical composition is a coformulation.

In some embodiments, the first pharmaceutical composition is administered with or on the same day as the second pharmaceutical composition

In some embodiments, the first pharmaceutical composition is administered simultaneously with the second pharmaceutical composition.

In some embodiments, the first pharmaceutical composition is administered at a first location of the subject and the second pharmaceutical composition is administered at a second location of the subject that is different than the first location.

In some embodiments, the first location is at an appendage of the subject and second location is at an opposing appendage of the subject,

In some embodiments, the first appendage is an arm and the second appendage is an arm.

In some embodiments, the first pharmaceutical composition and the second pharmaceutical composition are administered to the same location of the subject.

In some embodiments, the pharmaceutical composition is administered at a first time point and a second time point, wherein the second time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the pharmaceutical composition is administered at a third time point, wherein the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

In some embodiments, the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the first pharmaceutical composition is administered at a first time point and a second time point, wherein the second time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the first pharmaceutical composition is administered at a third time point, wherein the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

In some embodiments, the third time point is at least about, at most about or about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the second pharmaceutical composition is administered at the first time point.

In some embodiments, the second pharmaceutical composition is administered at the second time point.

In some embodiments, the second pharmaceutical composition is administered at the third time point.

In some embodiments, the second pharmaceutical composition is administered at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the second pharmaceutical composition is administered at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); wherein the pharmaceutical composition is administered at a first time point, a second time point and a third time point, wherein the second time point is at least about 2 days after the first time point and the third time point is at least about 2 days after the second time point.

In some embodiments, the second time point is at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at least about 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the second time point is at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at most about 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the second time point is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point is at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the second time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the second time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 days after the second time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point is at most about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the method further comprises administering to the subject: (ii)(a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b).

In some embodiments, the subject has an immunodeficiency.

In some embodiments, the subject has a B cell immunodeficiency.

In some embodiments, the pharmaceutical composition is administered prophylactically.

Provided herein is a pharmaceutical composition comprising: (i) a recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a recombinant polynucleotide encoding a 2019 SARS-CoV 2 spike protein or a variant or fragment thereof; wherein the ratio (e.g., mass ratio) of (i):(ii) is greater than 20:1 or less than 1:20.

In some embodiments, the ratio (e.g., mass ratio) of (i):(ii) is greater than 20:1, 30:1, 40:1, 50:1, 60:1, 70:1, 80:1, 90:1 or 100:1

In some embodiments, the ratio (e.g., mass ratio) of (i):(ii) is less than 1:20, 1:30, 1:40, 1:50, 1:60, 1:70, 1:80, 1:90 or 1:100.

Provided herein is a composition comprising: (i) a first pharmaceutical composition comprising a first recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second recombinant polynucleotide encoding a 2019 SARS-CoV 2 spike protein or a variant or fragment thereof; wherein the ratio (e.g., mass ratio) of the recombinant polynucleotide in (i) to the recombinant polynucleotide in (ii) is from 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the recombinant polynucleotide in (i) to the recombinant polynucleotide in (ii) is about 1:6. In some embodiments, the ratio (e.g., mass ratio) of the recombinant polynucleotide in (i) to the recombinant polynucleotide in (ii) is about 1:3. In some embodiments, the ratio (e.g., mass ratio) of the recombinant polynucleotide in (i) to the recombinant polynucleotide in (ii) is about 1:2.

In some embodiments, the recombinant polynucleotide in (i) is present at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the recombinant polynucleotide in (i) is present at a dose of from 0.05 microgram to 10 micrograms, or 0.1 microgram to 5 micrograms, or 0.3 microgram to 5 micrograms. In some embodiments, the recombinant polynucleotide in (i) is present at a dose of from 0.1 microgram to 20 micrograms or from 0.5 microgram to 15 micrograms.

In some embodiments, the recombinant polynucleotide in (ii) is present at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the recombinant polynucleotide in (ii) encompasses at least two recombinant polynucleotide, each encoding a SARS-CoV-2 S protein of a different strain or variant thereof. For example, in some embodiments, the recombinant polynucleotide in (ii) encompasses a recombinant polynucleotide encoding a SARS-CoV-2 protein of a Wuhan strain and a recombinant polynucleotide encoding a SARS-CoV-2 protein having one or more mutations that are characteristics of a SARS-CoV-2 variant (e.g., in some embodiments an Omicron variant such as, e.g., a Omicron BA.4 or BA.5).

In some embodiments, the recombinant polynucleotide in (i) is present at a dose of about 5 micrograms and the recombinant polynucleotide in (ii) is present at a dose of about 30 micrograms. In some embodiments, the recombinant polynucleotide in (i) is present at a dose of about 10 micrograms and the recombinant polynucleotide in (ii) is present at a dose of about 30 micrograms. In some embodiments, the recombinant polynucleotide in (i) is present at a dose of about 15 micrograms and the recombinant polynucleotide in (ii) is present at a dose of about 30 micrograms.

Provided herein is a pharmaceutical composition comprising a nanoparticle, wherein the nanoparticle comprises: (i) a first recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second recombinant polynucleotide encoding a 2019 SARS-CoV 2 spike protein or a variant or fragment thereof.

In some embodiments, the nanoparticle is present in the pharmaceutical composition at a dose of from 100 ng to 500 micrograms.

In some embodiments, the nanoparticle is present in the pharmaceutical composition at a dose of from 1 microgram to 100 micrograms.

In some embodiments, the nanoparticle is present in the pharmaceutical composition at a dose of from 1 microgram to 30 micrograms, 5 micrograms to 40 micrograms or 10 microgram to 50 micrograms.

In some embodiments, the nanoparticle is present in the pharmaceutical composition at a dose of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the ratio (e.g., mass ratio) of the first recombinant polynucleotide to the second recombinant polynucleotide is from about 1:50 to 50:1.

In some embodiments, the ratio (e.g., mass ratio) of the first recombinant polynucleotide to the second recombinant polynucleotide is from about 1:25 to 25:1.

In some embodiments, the ratio (e.g., mass ratio) of the first recombinant polynucleotide to the second recombinant polynucleotide is from about 1:10 to 10:1.

In some embodiments, the ratio (e.g., mass ratio) of the first recombinant polynucleotide to the second recombinant polynucleotide is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the first recombinant polynucleotide to the second recombinant polynucleotide is about 1:6. In some embodiments, the ratio (e.g., mass ratio) of the first recombinant polynucleotide to the second recombinant polynucleotide is about 1:3. In some embodiments, the ratio (e.g., mass ratio) of the first recombinant polynucleotide to the second recombinant polynucleotide is about 1:2.

In some embodiments, the first recombinant polynucleotide is present in a pharmaceutical composition at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the first recombinant polynucleotide is present in a pharmaceutical composition at a dose of from 0.05 microgram to 10 micrograms, or 0.1 microgram to 5 micrograms, or 0.3 microgram to 5 micrograms. In some embodiments, the first recombinant polynucleotide is present in a pharmaceutical composition at a dose of from 0.1 microgram to 20 micrograms or from 0.5 microgram to 15 micrograms.

In some embodiments, the second recombinant polynucleotide is present in a pharmaceutical composition at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the second recombinant polynucleotide encompasses at least two recombinant polynucleotide, each encoding a SARS-CoV-2 S protein of a different strain or variant thereof. For example, in some embodiments, the second recombinant polynucleotide encompasses a recombinant polynucleotide encoding a SARS-CoV-2 S protein of a Wuhan strain and a recombinant polynucleotide encoding a SARS-CoV-2 S protein having one or more mutations that are characteristics of a SARS-CoV-2 variant (e.g., in some embodiments an Omicron variant such as, e.g., an Omicron BA.1, BA.2, BA.4 or BA.5 variant).

Provided herein is a composition comprising: (i) a first pharmaceutical composition comprising a first nanoparticle, wherein the first nanoparticle comprises a recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second nanoparticle, wherein the second nanoparticle comprises a recombinant polynucleotide encoding a 2019 SARS-CoV 2 spike protein or a variant or fragment thereof.

In some embodiments, the ratio (e.g., mass ratio) of the recombinant polynucleotide in (i) to the recombinant polynucleotide in (ii) is from about 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8. In some embodiments, the ratio (e.g., mass ratio) of the recombinant polynucleotide in (i) to the recombinant polynucleotide in (ii) is about 1:6. In some embodiments, the ratio (e.g., mass ratio) of the recombinant polynucleotide in (i) to the recombinant polynucleotide in (ii) is about 1:3. In some embodiments, the ratio (e.g., mass ratio) of the recombinant polynucleotide in (i) to the recombinant polynucleotide in (ii) is about 1:2.

In some embodiments, the recombinant polynucleotide in (i) is present at a dose of from 0.1 microgram to 100 micrograms, or 1 microgram to 50 micrograms, or 1 microgram to 30 micrograms, or 1 microgram to 20 micrograms, or 3 micrograms to 20 micrograms, or 5 micrograms to 15 micrograms. In some embodiments, the recombinant polynucleotide in (i) is present at a dose of from 0.05 microgram to 10 micrograms, or 0.1 microgram to 5 micrograms, or 0.3 microgram to 5 micrograms. In some embodiments, the recombinant polynucleotide in (i) is present at a dose of from 0.1 microgram to 20 micrograms or from 0.5 microgram to 15 micrograms.

In some embodiments, the recombinant polynucleotide in (ii) is present at a dose of from 0.1 microgram to 100 micrograms, or 1 microgram to 100 micrograms, or 1 microgram to 30 micrograms, or 1 microgram to 20 micrograms, or 3 micrograms to 30 micrograms.

In some embodiments, the recombinant polynucleotide in (ii) encompasses at least two recombinant polynucleotide, each encoding a SARS-CoV-2 S protein of a different strain or variant thereof. For example, in some embodiments, the recombinant polynucleotide in (ii) encompasses a recombinant polynucleotide encoding a SARS-CoV-2 protein of a Wuhan strain and a recombinant polynucleotide encoding a SARS-CoV-2 protein having one or more mutations that are characteristics of a SARS-CoV-2 variant (e.g., in some embodiments an Omicron variant such as, e.g., a Omicron BA.4 or BA.5).

In some embodiments, the first nanoparticle is present in the first pharmaceutical composition at a dose of from about 100 ng to 500 micrograms.

In some embodiments, the first nanoparticle is present in the first pharmaceutical composition at a dose of from about 1 microgram to 100 micrograms.

In some embodiments, the first nanoparticle is present in the first pharmaceutical composition at a dose of from about 1 microgram to 30 micrograms, 5 micrograms to 40 micrograms or 10 microgram to 50 micrograms.

In some embodiments, the first nanoparticle is present in the first pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the second nanoparticle is present in the second pharmaceutical composition at a dose of from about 100 ng to 500 micrograms.

In some embodiments, the second nanoparticle is present in the second pharmaceutical composition at a dose of from about 1 microgram to 100 micrograms.

In some embodiments, the second nanoparticle is present in the second pharmaceutical composition at a dose of from about 1 microgram to 30 micrograms, 5 micrograms to 40 micrograms or 10 microgram to 50 micrograms.

In some embodiments, the second nanoparticle is present in the second pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the recombinant polynucleotide in (i) is present in the first pharmaceutical composition at a dose of from about 50 ng to 250 micrograms.

In some embodiments, the recombinant polynucleotide in (i) is present in the first pharmaceutical composition at a dose of from about 0.5 to 50 micrograms.

In some embodiments, the recombinant polynucleotide in (i) is present in the first pharmaceutical composition at a dose of from about 0.5 microgram to 15 micrograms, 2.5 micrograms to 20 micrograms or 5 microgram to 25 micrograms.

In some embodiments, the recombinant polynucleotide in (i) is present in the first pharmaceutical composition at a dose of about 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450 or 500 micrograms.

In some embodiments, the recombinant polynucleotide in (ii) is present in the second pharmaceutical composition at a dose of from about 50 ng to 250 micrograms.

In some embodiments, the recombinant polynucleotide in (ii) is present in the second pharmaceutical composition at a dose of from about 0.5 to 50 micrograms.

In some embodiments, the recombinant polynucleotide in (ii) is present in the second pharmaceutical composition at a dose of from about 0.5 microgram to 15 micrograms, 2.5 micrograms to 20 micrograms or 5 microgram to 25 micrograms.

In some embodiments, the recombinant polynucleotide in (ii) is present in the second pharmaceutical composition at a dose of about 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450 or 500 micrograms.

In some embodiments, the nanoparticle is a lipid nanoparticle.

Provided herein is a method of treating or preventing an infection by a virus or treating a respiratory disease or condition associated with an infection by a virus comprising administering to a subject in need thereof a pharmaceutical composition comprising: (i) a polypeptide comprising at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (ii) a polynucleotide encoding a polypeptide, wherein the polypeptide comprises at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (iii) a T cell receptor (TCR) or a T cell comprising the TCR, wherein the TCR binds to an epitope sequence of the polypeptide in complex with a corresponding HLA class I or class II molecule; (iv) an antigen presenting cell comprising (i) or (ii); or (v) an antibody or B cell comprising the antibody, wherein the antibody binds to an epitope sequence of the polypeptide.

In some embodiments, the subject has an immunodeficiency.

In some embodiments, the subject has a B cell immunodeficiency.

In some embodiments, the subject has a reduced ability to produce an antibody response to an antigen compared to a subject without an immunodeficiency.

In some embodiments, the subject has a reduced ability to produce an antibody response to a vaccination compared to a subject without an immunodeficiency.

In some embodiments, the subject has a reduced ability to produce an anti-spike protein antibody response and/or an anti-RBD antibody response compared to a subject without an immunodeficiency.

In some embodiments, the subject can produce a T cell response or does not have a reduced ability to produce a T cell response compared to a subject without an immunodeficiency.

In some embodiments, the pharmaceutical composition is protective against a variant of 2019 SARS CoV-2.

In some embodiments, the variant of 2019 SARS CoV-2 is alpha, beta, gamma, delta, epsilon, zeta, eta, theta, iota, kappa or lambda.

In some embodiments, the subject produces a T cell response to an epitope of the polypeptide.

In some embodiments, the subject produces a T cell response to the epitope sequence from ORF1ab, the epitope sequence from membrane glycoprotein (M) and/or the epitope sequence from nucleocapsid phosphoprotein (N).

In some embodiments, the subject is an organ transplant recipient.

In some embodiments, the organ transplant recipient is a sold organ transplant recipient, a stem cell transplant recipient or a bone marrow transplant recipient.

In some embodiments, the subject received an organ transplant less than 1 year, less than 6 months or less than 3 months after the pharmaceutical composition is administered.

In some embodiments, the subject is expected to receive an organ transplant less than 1 year, less than 6 months or less than 3 months prior to the pharmaceutical composition being administered.

In some embodiments, the subject has a cancer.

In some embodiments, the cancer is a B cell cancer.

In some embodiments, the B cell cancer is a B cell lymphoma or a B cell leukemia.

In some embodiments, the subject has an autoimmune disease or condition.

In some embodiments, the autoimmune disease or condition is Addison disease, Anti-NMDA receptor encephalitis, antisynthetase syndrome, Aplastic anemia, autoimmune anemias, Autoimmune hemolytic anemia, Autoimmune pancreatitis, Behcet's Disease, bullous skin disorders, Celiac disease-sprue, chronic fatigue syndrome, Chronic inflammatory demyelinating polyneuropathy, chronic lymphocytic leukemia, Crohn's disease, Dermatomyositis, Devic's disease, Erythroblastopenia, Evans syndrome, Focal segmental glomerulosclerosis, Granulomatosis with polyangiitis, Graves disease, Graves' ophthalmopathy, Guillain-Barre syndrome, Hashimoto thyroiditis, idiopathic thrombocytopenia purpura (ITP), IgA nephropathy, IgA-mediated autoimmune diseases, IgG4-related disease, Inflammatory bowel disease, Juvenile idiopathic arthritis, Multiple sclerosis, Myasthenia gravis, myeloma, non-Hodgkin's lymphoma, Opsoclonus myoclonus syndrome (OMS), Pemphigoid, Pemphigus, pemphigus vulgaris, Pernicious anemia, polymyositis, Psoriasis, pure red cell aplasia, Reactive arthritis, Rheumatoid arthritis, Sarcoidosis, scleroderma, Sjögren syndrome, Systemic lupus erythematosus, Thrombocytopenia purpura, Thrombotic thrombocytopeniaurpura, Type I diabetes, Ulcerative colitis, Vasculitis and Vitiligo.

In some embodiments, the subject has congenital agammaglobulinemia or congenital IgA deficiency.

In some embodiments, the subject has HIV or AIDS. In some embodiments, the subject has an age-related decline in immunity, immunosenescence, multifactoral immunodeficiency, or is an elderly or older adult with an age-related immunodeficiency.

In some embodiments, the subject is receiving an immunosuppressive agent or has received an immunosuppressive agent less than 1 year, less than 6 months or less than 3 months prior to the administering of the pharmaceutical composition.

In some embodiments, the immunosuppressive agent is abatacept, abrilumab, acalabrutinib, adalimumab, adrenocorticotropic hormone, agatolimod sodium, aldesleukin, alefacept, alemtuzumab, alisertib, alvespimycin hydrochloride, alvocidib, ambrisentan, aminocamptothecin, amiselimod, anakinra, andecaliximab, andrographolides, anifrolumab, antithymocyte Ig, apatinib, apelisib, asparaginase, atacicept, atezolizumab, avelumab, azacitidine, azathioprine, bafetinib, baminercept, baricitinib, basiliximab, becatecarin, begelomab, belatacept, belimumab, bemcentinib, bendamustine, bendamustine, betalutin with lilotomab, bevacizumab, BIIB033, BIIB059, BIIB061, bimekizumab, binimetinib, bleomycin, blinatumomab, bortezomib, brentuximab vedotin, bryostatin 1, bucillamine, buparlisib, busulfan, canakinumab, capecitabine, carboplatin, carfilzomib, carmustine, cediranib maleate, cemiplimab, ceralifimod, cerdulatinib, certolizumab, cetuximab, chidamide, chlorambucil, cilengitide, cirmtuzumab, cisplatin, cladribine, clazakizumab, clemastine, clioquinol, corticosteroids, cyclophosphamide, cyclosporine, cytarabine, cytotoxic chemotherapy, daclizumab, dalfampridine, daprolizumab pegol, daratumumab, dasatinib, defactinib, defibrotide, denosumab, dexamethasone, diacerein, dimethyl fumarate, dinaciclib, diroximel fumarate, doxorubicin, doxorubicin, durvalumab, duvelisib, duvortuxizumab, eculizumab, efalizumab, eftilagimod alpha, a neuropeptide combination of metenkefalin and tridecactide, elezanumab, elotuzumab, encorafenib, enfuvirtida, entinostat, entospletinib, enzastaurin, epacadostat, epirubicin, epratuzumab, eritoran tetrasodium, etanercept, etoposide, etrolizumab, everolimus, evobrutinib, filgotinib, fingolimod, firategrast, fludarabine, fluorouracil, fontolizumab, forodesine hydrochloride, fostamatinib, galunisertib, ganetespib, ganitumab, gemcitabine, gemtuzumab ozogamicin, gerilimzumab, glasdegib, glassia, glatiramer acetate, glembatumumab vedotin, glesatinib, golimumab, guadecitabine, hydrocortisone, hydroxychloroquine sulfate, hydroxyurea, ibritumomab tiuxetan, ibrutinib, ibudilast, idarubicin, idebenone, idelalisib, ifosfamide, iguratimod, imatinib, imexon, infliximab, inotuzumab ozogamicin, interferon alfa-2, interferon beta-la, interferon beta-1b, interferon gamma-1, ipilimumab, irofulven, isatuximab, ispinesib, itacitinib, ixazomib, lapatinib, laquinimod, laromustine, ld-aminopterin, leflunomide, lenalidomide, lenvatinib, letrozole, levamisole, levocabastine, lipoic acid, lirilumab, lonafarnib, lumiliximab, maraviroc, masitinib, mavrilimumab, melphalan, mercaptopurine, methotrexate, methoxsalen, methylprednisone, milatuzumab, mitoxantrone, mizoribine, mocetinostat, monalizumab, mosunetuzumab, motesanib diphosphate, moxetumomab pasudotox, muromonab-CD3, mycophenolate mofetil, mycophenolic acid, namilumab, natalizumab, navitoclax, neihulizumab, nerispirdine, neurovax, niraparib, nivolumab, obatoclax mesylate, obinutuzumab, oblimersen sodium, ocrelizumab, ofatumumab, olokizumab, opicinumab, oprelvekin, osimertinib, otelixizumab, oxaliplatin, oxcarbazepine, ozanimod, paclitaxel, pacritinib, palifermin, panobinostat, pazopanib, peficitinib, pegfilgrastim, peginterferon beta-la, pegsunercept (peg stnf-ri), pembrolizumab, pemetrexed, penclomedine, pentostatin, perifosine, pevonedistat, pexidartinib, picoplatin, pidilizumab, pivanex, pixantrone, pleneva, plovamer acetate, polatuzumab vedotin, pomalidomide, ponatinib, ponesimod, prednisone/prednisolone, pyroxamide, ravulizimab-cwvz, recombinant il-12, relatlimab, rhigf-1, rhigm22, rigosertib, rilonacept, ritonavir, rituximab, ruxolitinib, sarilumab, secukinumab, selumetinib, simvastatin, sintilimab, siplizumab, siponimod, sirolimus (rapamycin), sirukumab, sitravatinib, sonidegib, sorafenib, sotrastaurin acetate, sunitinib, sunphenon epigallocatechin-gallate, tabalumab, tacrolimus, talabostat mesylate, talacotuzumab, tanespimycin, tegafur/gimeracil/oteracil, temozolomide, temsirolimus, tenalisib, terameprocol, teriflunomide, thalidomide, thiarabine, thiotepa, tipifarnib, tirabrutinib, tislelizumab, tivozanib, tocilizumab, tofacitinib, tregalizumab, tremelimumab, treosulfan, ublituximab, umbralisib, upadacitinib, urelumab, ustekinumab, varlilumab, vatelizumab, vedolizumab, veliparib, veltuzumab, venetoclax, vinblastine, vincristine, vinorelbine ditartrate, visilizumab, vismodegib, vistusertib, voriconazole, vorinostat, vosaroxin, ziv-aflibercept or any combination thereof.

In some embodiments, the immunosuppressive agent is A2aR antagonist, Akt inhibitor, anti CD20, Anti-amyloidotic (AA) Agent, anti-CD37 protein therapeutic, anti-CTLA4 mAb, Anti-CXCR4, anti-huCD40 mAb, anti-LAG3 mAb, anti-PD-1 mAb, anti-PD-L1 agent, anti-PD-L1 agent, anti-PD-L1 mAb, anti-TGFb mAb, anti-TIGIT mAb, anti-TIM-3 mAb, Aurora kinase inhibitor, Bcl-2 Inhibitor, bifunctional fusion protein targeting TGFb and PD-L1, bispecific anti-PD-1 and anti-LAG3 mAb, CD1d ligand, CD40 agonist, Complement C5a inhibitor, CSF1R inhibitor, EZH2 inhibitor, FGFR3 inhibitor, FGFR4 inhibitor, FGFrR3 inhibitor, glucocorticoid-induced tumor necrosis factor receptor-related gene agonist, glutaminase inhibitor, Human monoclonal antibody against IL-12, ICOS agonist, IDO1 inhibitor, IL2 mutein, IL2 receptor agonist, MEK inhibitor, multitargeted receptor tyrosine kinase inhibitor, neutrophil elastase inhibitor, Notch Inhibitor, p38 MAPK inhibitor, PD-1 inhibitor, recombinant human Flt3L, ROCK inhibitor, selective sphingosine-1-phosphate receptor modulator, Src kinase inhibitor, TLR4 agonist, TLR9 agonist, or any combination thereof.

In some embodiments, the subject is greater than 55, 56, 57, 58, 59, 60, 65, 70, 75 or 80 years of age.

In some embodiments, the polypeptide comprises (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N).

In some embodiments, the sequence comprising an epitope sequence from ORF1ab is C-terminal to the sequence comprising an epitope sequence from nucleocapsid phosphoprotein (N).

In some embodiments, the sequence comprising an epitope sequence from ORF1ab is N-terminal to the sequence comprising an epitope sequence from membrane glycoprotein (M).

In some embodiments, the sequence comprising an epitope sequence from nucleocapsid phosphoprotein (N) is N-terminal to the sequence comprising an epitope sequence from membrane glycoprotein (M).

In some embodiments, the polypeptide comprises (a) 2, 3, 4, 5, 6, 7, 8, 9 or 10 or more epitope sequences from ORF1ab, (b) a sequence comprising an epitope sequence from membrane glycoprotein (M) and (c) a sequence comprising an epitope sequence from nucleocapsid phosphoprotein (N).

In some embodiments, the epitope sequence from ORF1ab is an epitope sequence from a non-structural protein (NSP).

In some embodiments, the non-structural protein (NSP) is selected from the group consisting of NSP1, NSP2, NSP3, NSP4 and combinations thereof.

In some embodiments, the polypeptide comprises a sequence comprising an epitope sequence from NSP1, a sequence comprising an epitope sequence from NSP2, a sequence comprising an epitope sequence from NSP3 and a sequence comprising an epitope sequence from NSP4.

In some embodiments, the epitope sequence from ORF1ab is selected from the group consisting of YLFDESGEFKL, YLFDESGEF, FGDDTVIEV, QLMCQPILL, TTDPSFLGRY, PTDNYITTY, PSFLGRY, AEAELAKNV, KTIQPRVEK and any combination thereof.

In some embodiments, the epitope sequence from nucleocapsid glycoprotein (N) is LLLDRLNQL.

In some embodiments, the epitope sequence from membrane phosphoprotein (M) is VATSRTLSY.

In some embodiments, the polypeptide comprises an epitope sequence from nucleocapsid glycoprotein (N) that is LLLDRLNQL and an epitope sequence from membrane phosphoprotein (M) that is VATSRTLSY.

In some embodiments, the polypeptide comprises (a) each of the following epitope sequences from ORF1ab: YLFDESGEFKL, YLFDESGEF, FGDDTVIEV, QLMCQPILL, TTDPSFLGRY, PTDNYITTY, PSFLGRY, AEAELAKNV, KTIQPRVEK; (b) an epitope sequence from nucleocapsid glycoprotein (N) that is LLLDRLNQL; and (c) an epitope sequence from membrane phosphoprotein (M) that is VATSRTLSY.

In some embodiments, the sequence comprising an epitope sequence from ORF1ab is selected from the group fragments thereof: consisting of the following sequences or

MVTNNTFTLKVPHVGEIPVAYRKVLLKTIQPRVEKYLFDESGEFKLSEVG
PEHSLAEYYIFFASFYY; MVTNNTFTLKVPHVGEIPVAYRKVLLKTIQP
RVEKYLFDESGEFKLSEVGPEHSLAEY; APKEIIFLEGETLFGDDTVIE
VAIILASFSAST; APKEIIFLEGETLFGDDTVIEV; HTTDPSFLGRYM
SALFADDLNQLTGYHTDFSSEIIGYQLMCQPILLAEAELAKNVSLILGTV
SWNL; TTDPSFLGRYMSALFADDLNQLTGYHTDFSSEIIGYQLMCQPIL
LAEAELAKNVSLILGTVSWNL; LLSAGIFGAITDVFYKENSYKVPTDNY
ITTY; and combinations thereof.

In some embodiments, the sequence comprising an epitope sequence from membrane glycoprotein (M) is selected from the group consisting of the following sequences or fragments thereof:

ADSNGTITVEELKKLLEQWNLVIGFLFLTWICLLQFAYANRNRFLYIIKL
IFLWLLWPVTLACFVLAAVYRINWITGGIAIAMACLVGLMWLSYFIASFR
LFARTRSMWSFNPETNILLNVPLHGTILTRPLLESELVIGAVILRGHLRI
AGHHLGRCDIKDLPKEITVATSRTLSYYKLGASQRVAGDSGFAAYSRYRI
GNYKLNTDHSSSSDNIALLVQ; FAYANRNRFLYIIKLIFLWLLWPVTLA
CFVLAAVYRINWITGGIAIAMACLVGLMWLSYFIASFRLF; LGRCDIKD
LPKEITVATSRTLSYYKLGASQRVA; KLLEQWNLVIGF; NRNRFLYII
KLIFLWLLWPVTLACFVLAAVY; SELVIGAVILRGHLRIAGHHLGR;
VATSRTLSYYKLGASQRV; GLMWLSYF; and combinations
thereof.

In some embodiments, the sequence comprising an epitope sequence from nucleocapsid phosphoprotein (N) is selected from the group consisting of the following sequences or fragments thereof:

KDLSPRWYFYYLGTGPEAGLPYGANKDGIIWVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQA; RMAGNGGDAALALLLLDRLNQLESKMSGKG
QQQ; YKHWPQIAQFAPSASAFFGMSRIGMEVTPSGTWLTYTGAIKLDDK
DPNFKDQVILLNKHIDAYKTFP; SPARMAGNGGDAALALLLLDRLNQLE
SKMSGKGQQQQGQTVTKKSAAEASKKPRQKRTATKAYNVTQAFGRRGPEQ
TQGNFGDQELIRQGTDYKHWPQIAQFAPSASAFFGMSRIGMEVTPSGTWL
TYTGAIKLDDKDPNFKDQVILLNKHIDAYKTFPPTEPKKDK and
combinations thereof.

In some embodiments, the polypeptide comprises one or more linker sequences.

In some embodiments, the one or more linker sequences are selected from the group consisting of GGSGGGGSGG, GGSLGGGGSG.

In some embodiments, the one or more linker sequences comprise cleavage sequences.

In some embodiments, the one or more cleavage sequences are selected from the group consisting of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA.

In some embodiments, the polypeptide comprises a transmembrane domain sequence.

In some embodiments, the transmembrane domain sequence is C-terminal to the sequence comprising an epitope sequence from ORF1ab, the sequence comprising an epitope sequence from membrane glycoprotein (M) and the sequence comprising an epitope sequence from nucleocapsid phosphoprotein (N).

In some embodiments, the transmembrane domain sequence is EQYIKWPWYIWLGFIAGLIAIVMVTIMLCCMTSCCSCLKGCCSCGSCCKFDEDDSEPVLKGVKL HYT.

In some embodiments, the polypeptide comprises a secretory signal sequence (SEC sequence).

In some embodiments, the SEC sequence is N-terminal to the sequence comprising an epitope sequence from ORF1ab, the sequence comprising an epitope sequence from membrane glycoprotein (M) and the sequence comprising an epitope sequence from nucleocapsid phosphoprotein (N).

In some embodiments, the SEC sequence is MFVFLVLLPLVSSQCVNLT.

In some embodiments, the composition comprises the polynucleotide encoding the polypeptide.

In some embodiments, the polynucleotide is an mRNA.

In some embodiments, the polynucleotide comprises a codon optimized sequence for expression in a human.

In some embodiments, the polynucleotide comprises a dEarI-hAg sequence.

In some embodiments, the dEarI-hAg sequence is ATTCTTCTGGTCCCCACAGACTCAGAGAGAACCC, optionally wherein each T is a U.

In some embodiments, the polynucleotide comprises a Kozak sequence.

In some embodiments, the Kozak sequence is GCCACC.

In some embodiments, the polynucleotide comprises an F element sequence.

In some embodiments, the F element sequence is a 3 UTR of amino-terminal enhancer of split (AES).

In some embodiments, the F element sequence is CTGGTACTGCATGCACGCAATGCTAGCTGCCCCTTTCCCGTCCTGGGTACCCCGAGTCTCCC CCGACCTCGGGTCCCAGGTATGCTCCCACCTCCACCTGCCCCACTCACCACCTCTGCTAGTTC CAGACACCTCC, optionally wherein each T is a U.

In some embodiments, the polynucleotide comprises an I element sequence.

In some embodiments, the I element sequence is a 3′ UTR of mitochondrially encoded 12S rRNA (mtRNR1).

In some sequence is embodiments, the I element CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA

ATTTCGTGCCAGCCACACC, optionally wherein each T is a U.

In some embodiments, the polynucleotide comprises a poly A sequence.

In some embodiments, the poly A sequence is AAAAAAAAAAAAAAAAAAAAAAAAAAAAAAGCATATGACTAAAAAAAAAAAAAAAAAAA AAAAAAAAAAAAAAAAAAAAAAAAAAAAAAAAAAAAAAAAAAAAAAAAAAA, optionally wherein each T is a U.

In some embodiments, each of the epitope sequences from the ORF1ab, the membrane glycoprotein, and the nucleocapsid phosphoprotein are from 2019 SARS-CoV-2.

In some embodiments, one or more or each epitope elicits a T cell response.

In some embodiments, one or more or each epitope has been observed by mass spectrometry as being presented by an HLA molecule.

In some embodiments, the composition comprises (i) a polypeptide with at least 70%, at least 75%, at least 80%, at least 85%, at least 90%, at least 95% or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; (ii) a polynucleotide encoding a polypeptide with at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or (iii) a polynucleotide with at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS C6n2, RS C7n2, RS C8n2, RS C5n2full, RS Con2full, RS C7n2full, RS C8n2full, RS C7n4, RS C7n4full.

In some embodiments, the composition comprises (i) a polypeptide with at least 70%, 80%, 90% or 100% sequence identity to a sequence selected from the group consisting of RS C7p1, RS C7p2, RS C7p2full, RS C7p4, and RS C7p4full; or (iii) a polynucleotide with at least at least 70%, at least 75%, at least 80%, at least 85%, at least 90%, at least 95%, or 100% sequence identity to a sequence selected from the group consisting of SEQ ID NOs: RS C7n1, RS C7n2, RS C7n2full, RS C7n4, RS and C7n4full.

In some embodiments, the pharmaceutical composition comprises a pharmaceutically acceptable excipient, carrier, or diluent.

Provided herein is a method of treating or preventing an infection by a virus or treating a respiratory disease or condition associated with an infection by a virus comprising administering to a subject in need thereof a pharmaceutical composition comprising: (i) a polypeptide comprising an epitope sequence of Table 1A, Table 1B, Table 1C, Table 2Ai, Table 2Aii, Table 2B and/or Table 16; (ii) a polynucleotide encoding the polypeptide comprising an epitope sequence of Table 1A, Table 1B, Table 1C, Table 2Ai, Table 2Aii, Table 2B and/or Table 16; (iii) a T cell receptor (TCR) or a T cell comprising the TCR, wherein the TCR binds to the epitope sequence in complex with a corresponding HLA class I or class II molecule; (iv) an antigen presenting cell comprising (i) or (ii); or (v) an antibody or B cell comprising the antibody, wherein the antibody binds to the epitope sequence.

In some embodiments, the subject has an immunodeficiency.

In some embodiments, the subject has a B cell immunodeficiency.

In some embodiments, the epitope sequence comprises one or more or each of the following: YLFDESGEFKL, YLFDESGEF, FGDDTVIEV, LLLDRLNQL, QLMCQPILL, TTDPSFLGRY, PTDNYITTY, PSFLGRY, AEAELAKNV, VATSRTLSY and KTIQPRVEK.

In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV, FGADPIHSL, NYNYLYRLF, KYIKWPWYI, KWPWYIWLGF, LPFNDGVYF, QPTESIVRF, IPFAMQMAY, YLQPRTFLL and RLQSLQTYV.

In some embodiments, the epitope sequence is from an orflab protein.

In some embodiments, the epitope sequence is from an orfla protein

In some embodiments, the epitope sequence is from a surface glycoprotein(S) or a shifted reading frame thereof.

In some embodiments, the epitope sequence is from a nucleocapsid phosphoprotein (N).

In some embodiments, the epitope sequence is from an ORF3a protein.

In some embodiments, the epitope sequence is from a membrane glycoprotein (M).

In some embodiments, the epitope sequence is from an ORF7a protein.

In some embodiments, the epitope sequence is from an ORF8 protein.

In some embodiments, the epitope sequence is from an envelope protein (E).

In some embodiments, the epitope sequence is from an ORF6 protein.

In some embodiments, the epitope sequence is from an ORF7b protein.

In some embodiments, the epitope sequence is from an ORF10 protein.

In some embodiments, the epitope sequence is from an ORF9b protein.

Provided herein is a method of treating or preventing an infection by a virus or treating a respiratory disease or condition associated with an infection by a virus comprising administering to a subject in need thereof a pharmaceutical composition comprising: a polypeptide having an amino acid sequence with at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15; or a recombinant polynucleotide encoding a polypeptide having an amino acid sequence with at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15.

In some embodiments, the subject has an immunodeficiency.

In some embodiments, the subject has a B cell immunodeficiency.

In some embodiments, the pharmaceutical composition comprises a polypeptide with at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or a polynucleotide encoding a polypeptide with at least 70%, at least 75%, at least 80%, at least 85%, at least 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full.

In some embodiments, the pharmaceutical composition comprises a polynucleotide with at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS Con2, RS C7n2, RS C8n2, RS C5n2full, RS Con2full, RS C7n2full, RS C8n2full, RS C7n4, and RS C7n4full.

In some embodiments, the polynucleotide is an mRNA.

In some embodiments, the pharmaceutical composition further comprises one or more lipids.

In some embodiments, the one or more lipids comprise a lipid nanoparticle (LNP).

In some embodiments, the LNP encapsulates the recombinant polynucleotide construct.

In some embodiments, the polypeptide is synthetic.

In some embodiments, the polypeptide is recombinant.

In some embodiments, the polypeptide is from 8-1000 amino acids in length.

In some embodiments, the epitope sequence binds to or is predicted to bind to an HLA class I or class II molecule with a KD of 1000 nM or less.

In some embodiments, the epitope sequence binds to or is predicted to bind to an HLA class I or class II molecule with a KD of 500 nM or less.

In some embodiments, the epitope sequence comprises a sequence of a viral protein expressed by a virus-infected cell of the subject.

In some embodiments, the virus is a coronavirus.

In some embodiments, the virus is 2019 SARS-CoV 2.

In some embodiments, an HLA molecule expressed by the subject is unknown at the time of administration.

In some embodiments, the ability of the virus to avoid escape of recognition by an immune system of the subject is less compared to the ability of the virus to avoid escape of recognition by an immune system of a subject administered a pharmaceutical composition containing an epitope from a single protein or epitopes from fewer proteins than in the pharmaceutical composition administered according to a method described herein.

In some embodiments, the subject expresses an HLA molecule encoded by an HLA allele of any one of Table 1A, Table 1B, Table 1C, Table 2Ai, Table 2Aii, Table 2B and Table 16 and the epitope sequence is an HLA allele-matched epitope sequence.

In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV and FGADPIHSL.

In some embodiments, the method further comprises administering to the subject an additional therapy for a 2019 SARS-CoV 2 viral infection.

In some embodiments, the method further comprises administering to the subject (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (a) or (b).

In some embodiments, the vaccine or therapeutic of (a) or (b) is administered to the subject once.

In some embodiments, the vaccine or therapeutic of (a) or (b) is administered to the subject more than once.

In some embodiments, the vaccine or therapeutic of (a) or (b) is administered at least two times, wherein the first administered dose is a priming dose, and the second and subsequent doses are booster dose(s). In some embodiments, the vaccine or therapeutic of (a) or (b) is administered at least three times, wherein the first administered dose is a priming dose, and the second, third, and subsequent doses are booster dose(s).

In some embodiments, the priming and the booster doses are administered at an interval of at least 21 days.

In some embodiments, an interval between two booster doses is at least 30 days, at least 60 days or at least 90 days.

In some embodiments, the vaccine or therapeutic is administered once each year.

In some embodiments, the vaccine or therapeutic is administered twice each year.

In some embodiments, the vaccine or therapeutic is administered at a high priming or loading dose for the first dose, and at a reduced boosting or maintenance dose for the subsequent doses.

In some embodiments, the subject receives a lower dose of or a lower frequency of a SARS-CoV spike vaccine than a subject receiving the SARS-CoV spike vaccine alone.

Provided herein is a method of treating or preventing an infection by a virus or treating a respiratory disease or condition associated with an infection by a virus comprising administering to a subject in need thereof a pharmaceutical composition comprising: (i) a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a recombinant polynucleotide encoding a 2019 SARS-CoV 2 spike protein or a variant or fragment thereof.

In some embodiments, provided herein is a method of treating or preventing an infection by a virus or treating a respiratory disease or condition associated with an infection by a virus comprising administering to a subject in need thereof: (i) a first pharmaceutical composition comprising a first recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second recombinant polynucleotide encoding a 2019 SARS-CoV 2 spike protein or a variant or fragment thereof.

In some embodiments, the ratio (e.g., mass ratio) of the recombinant polynucleotide in (i) to the recombinant polynucleotide in (ii) is from 20:1 to 1:20.

In some embodiments, the ratio (e.g., mass ratio) of the recombinant polynucleotide in (i) to the recombinant polynucleotide in (ii) is from 1:10 to 10:1.

In some embodiments, the ratio (e.g., mass ratio) of the recombinant polynucleotide in (i) to the recombinant polynucleotide in (ii) is from about 1:5 to 5:1.

In some embodiments, the ratio (e.g., mass ratio) of the recombinant polynucleotide in (i) to the recombinant polynucleotide in (ii) is from about 1:3 to 3:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

In some embodiments, a method of treating or preventing an infection comprises administering the first pharmaceutical composition (i) and the second pharmaceutical composition (ii), to a subject who has previously been administered one or more doses of a SARS-CoV-2 vaccine (e.g., a SARS-CoV-2 vaccine that comprises (a) a polypeptide that includes a SARS-CoV-2 spike protein, or a fragment or variant thereof, or (b) a recombinant polynucleotide comprising a sequence that encodes a SARS-CoV-2 spike protein or a fragment or variant thereof). In some embodiments, a method of treating or preventing an infection comprises administering the first pharmaceutical composition (i) and the second pharmaceutical composition (ii) to a subject who has previously been administered two or more (e.g., three) doses of a SARS-CoV-2 vaccine.

In some embodiments, provided herein is a method of treating or preventing an infection by a virus or treating a respiratory disease or condition associated with an infection by a virus comprising administering to a subject in need thereof a pharmaceutical composition comprising: (i) a first recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second recombinant polynucleotide encoding a 2019 SARS-CoV 2 spike protein or a variant or fragment thereof.

In some embodiments, the pharmaceutical composition comprises a nanoparticle, wherein the nanoparticle comprises the first recombinant polynucleotide and the second recombinant polynucleotide.

In some embodiments, the nanoparticle is present in the pharmaceutical composition at a dose of from 100 ng to 500 micrograms.

In some embodiments, the nanoparticle is present in the pharmaceutical composition at a dose of from 1 microgram to 100 micrograms.

In some embodiments, the nanoparticle is present in the pharmaceutical composition at a dose of from 1 microgram to 30 micrograms, 5 micrograms to 40 micrograms or 10 microgram to 50 micrograms.

In some embodiments, the nanoparticle is present in the pharmaceutical composition at a dose of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the ratio (e.g., mass ratio) of the first recombinant polynucleotide to the second recombinant polynucleotide is from about 1:50 to 50:1.

In some embodiments, the ratio (e.g., mass ratio) of the first recombinant polynucleotide to the second recombinant polynucleotide is from about 1:25 to 25:1.

In some embodiments, the ratio (e.g., mass ratio) of the first recombinant polynucleotide to the second recombinant polynucleotide is from about 1:10 to 10:1.

In some embodiments, the ratio (e.g., mass ratio) of the first recombinant polynucleotide to the second recombinant polynucleotide is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

Provided herein is a method of treating or preventing an infection by a virus or treating a respiratory disease or condition associated with an infection by a virus comprising administering to a subject in need thereof: (i) a first pharmaceutical composition comprising a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a recombinant polynucleotide encoding a 2019 SARS-CoV 2 spike protein or a variant or fragment thereof.

In some embodiments, the ratio (e.g., mass ratio) of the recombinant polynucleotide in (i) to the recombinant polynucleotide in (ii) is from about 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

In some embodiments, the first pharmaceutical composition comprises a first nanoparticle, wherein the first nanoparticle comprises the recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and wherein the second pharmaceutical composition comprises a second nanoparticle, wherein the second nanoparticle comprises the recombinant polynucleotide encoding a 2019 SARS-CoV 2 spike protein or a variant or fragment thereof.

In some embodiments, the first nanoparticle is present in the first pharmaceutical composition at a dose of from about 100 ng to 500 micrograms.

In some embodiments, the first nanoparticle is present in the first pharmaceutical composition at a dose of from about 1 microgram to 100 micrograms.

In some embodiments, the first nanoparticle is present in the first pharmaceutical composition at a dose of from about 1 microgram to 30 micrograms, 5 micrograms to 40 micrograms or 10 microgram to 50 micrograms.

In some embodiments, the first nanoparticle is present in the first pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the second nanoparticle is present in the second pharmaceutical composition at a dose of from about 100 ng to 500 micrograms.

In some embodiments, the second nanoparticle is present in the second pharmaceutical composition at a dose of from about 1 microgram to 100 micrograms.

In some embodiments, the second nanoparticle is present in the second pharmaceutical composition at a dose of from about 1 microgram to 30 micrograms, 5 micrograms to 40 micrograms or 10 microgram to 50 micrograms.

In some embodiments, the second nanoparticle is present in the second pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii)(a) a pharmaceutical composition comprising a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b);

wherein the subject receives a dose of (ii)(a) or (ii)(b) that is lower than a dose of (ii)(a) or (ii)(b) administered to a subject alone.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii)(a) a pharmaceutical composition comprising a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b);

wherein the subject receives a number of doses of (ii)(a) or (ii)(b) that is lower than a number of doses of (ii)(a) or (ii)(b) administered to a subject alone.

In some embodiments, the subject receives a dose of (ii)(a) or (ii)(b) that is at least 1.1, 1, 1.5, 2, 3, 4, 5, 6, 7, 8, 9, 10, 20, 30, 40, 50, 60, 70, 80, 90 or 100 times lower than a dose of (ii)(a) or (ii)(b) administered to a subject alone.

In some embodiments, the subject receives 1, 2, 3, 4, 5, 6, 7, 8, 9 or 10 fewer doses of (ii)(a) or (ii)(b) than the number of doses of (ii)(a) or (ii)(b) administered to a subject alone.

In some embodiments, the pharmaceutical composition of (i) is co-formulated with the pharmaceutical composition of (ii).

In some embodiments, the pharmaceutical composition of (i) is formulated separately from the pharmaceutical composition of (ii).

In some embodiments, the pharmaceutical composition of (i) is administered separately from the pharmaceutical composition of (ii).

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a dose of (i)(a) or (i)(b) that is lower than a dose of (i)(a) or (i)(b) administered to a subject alone.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a pharmaceutical composition comprising (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b); wherein the subject receives a number of doses of (i)(a) or (i)(b) that is lower than a number of doses of (i)(a) or (i)(b) administered to a subject alone.

In some embodiments, the subject receives a dose of (i)(a) or (i)(b) that is at least 1.1, 1, 1.5, 2, 3, 4, 5, 6, 7, 8, 9, 10, 20, 30, 40, 50, 60, 70, 80, 90 or 100 times lower than a dose of (i)(a) or (i)(b) administered to a subject alone.

In some embodiments, the subject receives 1, 2, 3, 4, 5, 6, 7, 8, 9 or 10 fewer doses of (i)(a) or (i)(b) than the number of doses of (i)(a) or (i)(b) administered to a subject alone.

In some embodiments, the pharmaceutical composition of (i) is co-formulated with the pharmaceutical composition of (ii).

In some embodiments, the pharmaceutical composition of (i) is formulated separately from the pharmaceutical composition of (ii).

In some embodiments, the pharmaceutical composition of (i) is administered separately from the pharmaceutical composition of (ii).

In some embodiments, the pharmaceutical composition is a coformulation.

In some embodiments, the first pharmaceutical composition is administered with or on the same day as the second pharmaceutical composition.

In some embodiments, the first pharmaceutical composition is administered simultaneously with the second pharmaceutical composition.

In some embodiments, the first pharmaceutical composition is administered at a first location of the subject and the second pharmaceutical composition is administered at a second location of the subject that is different than the first location.

In some embodiments, the first location is at an appendage of the subject and second location is at an opposing appendage of the subject,

In some embodiments, the first appendage is an arm and the second appendage is an arm.

In some embodiments, the first pharmaceutical composition and the second pharmaceutical composition are administered to the same location of the subject.

In some embodiments, the pharmaceutical composition is administered at a first time point and a second time point, wherein the second time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the pharmaceutical composition is administered at a third time point, wherein the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

In some embodiments, the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the first pharmaceutical composition is administered at a first time point and a second time point, wherein the second time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the first pharmaceutical composition is administered at a third time point, wherein the third time point is at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

In some embodiments, the third time point is at least about, at most about or about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the second pharmaceutical composition is administered at the first time point.

In some embodiments, the second pharmaceutical composition is administered at the second time point.

In some embodiments, the second pharmaceutical composition is administered at the third time point.

In some embodiments, the second pharmaceutical composition is administered at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the first time point.

In some embodiments, the second pharmaceutical composition is administered at least about, at most about or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the second time point; at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the second time point; or at least about, at most about or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months after the second time point.

Provided herein is a method of treating or preventing an infection by a virus or treating a respiratory disease or condition associated with an infection by a virus comprising administering to a subject in need thereof: (i) a pharmaceutical composition comprising (a) a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); or (b) a polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); wherein the pharmaceutical composition is administered at a first time point and a second time point, wherein the second time point is at least about 2 days after the first time point.

In some embodiments, the pharmaceutical composition is administered at a third time point, wherein the third time point is at least about 2 days after the second time point.

In some embodiments, the second time point is at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at least about 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the second time point is at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at most about 1, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the second time point is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the first time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point is at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the second time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after the second time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 days after the second time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the second time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the second time point.

In some embodiments, the third time point is at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point is at most about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the third time point about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days after the first time point, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 weeks after the first time point, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months after the first time point.

In some embodiments, the method further comprises administering to the subject: (ii)(a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (ii)(a) or (ii)(b).

In some embodiments, the subject has an immunodeficiency.

In some embodiments, the subject has a B cell immunodeficiency.

In some embodiments, the pharmaceutical composition is administered prophylactically.

Provided herein is a pharmaceutical composition comprising: (i) a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a recombinant polynucleotide encoding a 2019 SARS-CoV 2 spike protein or a variant or fragment thereof.

In some embodiments, the ratio (e.g., mass ratio) of (i):(ii) is from 20:1 to 1:20.

In some embodiments, the ratio (e.g., mass ratio) of (i):(ii) is less than 20:1, 30:1, 40:1, 50:1, 60:1, 70:1, 80:1, 90:1 or 100:1.

In some embodiments, the ratio (e.g., mass ratio) of (i):(ii) is greater than 1:20, 1:30, 1:40, 1:50, 1:60, 1:70, 1:80, 1:90 or 1:100.

Provided herein is a composition comprising: (i) a first pharmaceutical composition comprising a first recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a second recombinant polynucleotide encoding a 2019 SARS-CoV 2 spike protein or a variant or fragment thereof.

In some embodiments, the ratio (e.g., mass ratio) of the recombinant polynucleotide in (i) to the recombinant polynucleotide in (ii) is from 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

Provided herein is a pharmaceutical composition comprising: (i) a first recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second recombinant polynucleotide encoding a 2019 SARS-CoV 2 spike protein or a variant or fragment thereof.

In some embodiments, the pharmaceutical composition comprises a nanoparticle, wherein the nanoparticle comprises the first recombinant polynucleotide and the second recombinant polynucleotide.

In some embodiments, the nanoparticle is present in the pharmaceutical composition at a dose of from 100 ng to 500 micrograms.

In some embodiments, the nanoparticle is present in the pharmaceutical composition at a dose of from 1 microgram to 100 micrograms.

In some embodiments, the nanoparticle is present in the pharmaceutical composition at a dose of from 1 microgram to 30 micrograms, 5 micrograms to 40 micrograms or 10 microgram to 50 micrograms.

In some embodiments, the nanoparticle is present in the pharmaceutical composition at a dose of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the ratio (e.g., mass ratio) of the first recombinant polynucleotide to the second recombinant polynucleotide is from about 1:50 to 50:1.

In some embodiments, the ratio (e.g., mass ratio) of the first recombinant polynucleotide to the second recombinant polynucleotide is from about 1:25 to 25:1.

In some embodiments, the ratio (e.g., mass ratio) of the first recombinant polynucleotide to the second recombinant polynucleotide is from about 1:10 to 10:1.

In some embodiments, the ratio (e.g., mass ratio) of the first recombinant polynucleotide to the second recombinant polynucleotide is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

Provided herein is a composition comprising: (i) a first pharmaceutical composition comprising a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a second pharmaceutical composition comprising a recombinant polynucleotide encoding a 2019 SARS-CoV 2 spike protein or a variant or fragment thereof.

In some embodiments, the first pharmaceutical composition comprises a first nanoparticle, wherein the first nanoparticle comprises the recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and wherein the second pharmaceutical composition comprises a second nanoparticle, wherein the second nanoparticle comprises the recombinant polynucleotide encoding a 2019 SARS-CoV 2 spike protein or a variant or fragment thereof.

In some embodiments, the ratio (e.g., mass ratio) of the recombinant polynucleotide in (i) to the recombinant polynucleotide in (ii) is from about 1:50 to 50:1.

In some embodiments, the ratio (e.g., mass ratio) of the recombinant polynucleotide in (i) to the recombinant polynucleotide in (ii) is from about 1:25 to 25:1.

In some embodiments, the ratio (e.g., mass ratio) of the recombinant polynucleotide in (i) to the recombinant polynucleotide in (ii) is from about 1:10 to 10:1.

In some embodiments, the ratio (e.g., mass ratio) of the recombinant polynucleotide in (i) to the recombinant polynucleotide in (ii) is about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8.

In some embodiments, the first nanoparticle is present in the first pharmaceutical composition at a dose of from about 100 ng to 500 micrograms.

In some embodiments, the first nanoparticle is present in the first pharmaceutical composition at a dose of from about 1 microgram to 100 micrograms.

In some embodiments, the first nanoparticle is present in the first pharmaceutical composition at a dose of from about 1 microgram to 30 micrograms, 5 micrograms to 40 micrograms or 10 microgram to 50 micrograms.

In some embodiments, the first nanoparticle is present in the first pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the second nanoparticle is present in the second pharmaceutical composition at a dose of from about 100 ng to 500 micrograms.

In some embodiments, the second nanoparticle is present in the second pharmaceutical composition at a dose of from about 1 microgram to 100 micrograms.

In some embodiments, the second nanoparticle is present in the second pharmaceutical composition at a dose of from about 1 microgram to 30 micrograms, 5 micrograms to 40 micrograms or 10 microgram to 50 micrograms.

In some embodiments, the second nanoparticle is present in the second pharmaceutical composition at a dose of about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900 or 1,000 micrograms.

In some embodiments, the recombinant polynucleotide in (i) is present in the first pharmaceutical composition at a dose of from about 50 ng to 250 micrograms.

In some embodiments, the recombinant polynucleotide in (i) is present in the first pharmaceutical composition at a dose of from about 0.5 to 50 micrograms.

In some embodiments, the recombinant polynucleotide in (i) is present in the first pharmaceutical composition at a dose of from about 0.5 microgram to 15 micrograms, 2.5 micrograms to 20 micrograms or 5 microgram to 25 micrograms.

In some embodiments, the recombinant polynucleotide in (i) is present in the first pharmaceutical composition at a dose of about 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450 or 500 micrograms.

In some embodiments, the recombinant polynucleotide in (ii) is present in the second pharmaceutical composition at a dose of from about 50 ng to 250 micrograms.

In some embodiments, the recombinant polynucleotide in (ii) is present in the second pharmaceutical composition at a dose of from about 0.5 to 50 micrograms.

In some embodiments, the recombinant polynucleotide in (ii) is present in the second pharmaceutical composition at a dose of from about 0.5 microgram to 15 micrograms, 2.5 micrograms to 20 micrograms or 5 microgram to 25 micrograms.

In some embodiments, the recombinant polynucleotide in (ii) is present in the second pharmaceutical composition at a dose of about 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450 or 500 micrograms.

In some embodiments, the nanoparticle is a lipid nanoparticle.

Provided herein is a composition comprising: (i) a polypeptide comprising at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (ii) a polynucleotide encoding a polypeptide, wherein the polypeptide comprises at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N); (iii) a T cell receptor (TCR) or a T cell comprising the TCR, wherein the TCR binds to an epitope sequence of the polypeptide in complex with a corresponding HLA class I or class II molecule; (iv) an antigen presenting cell comprising (i) or (ii); or (v) an antibody or B cell comprising the antibody, wherein the antibody binds to an epitope sequence of the polypeptide; and a pharmaceutically acceptable excipient.

In some embodiments, the polypeptide comprises (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N). In some embodiments, the sequence comprising an epitope sequence from ORF1ab is C-terminal to the sequence comprising an epitope sequence from nucleocapsid phosphoprotein (N). In some embodiments, the sequence comprising an epitope sequence from ORF1ab is N-terminal to the sequence comprising an epitope sequence from membrane glycoprotein (M). In some embodiments, the sequence comprising an epitope sequence from nucleocapsid phosphoprotein (N) is N-terminal to the sequence comprising an epitope sequence from membrane glycoprotein (M).

In some embodiments, the polypeptide comprises at least two of the following (a) a sequence comprising an epitope sequence from ORF1ab, (b) a sequence comprising an epitope sequence from membrane glycoprotein (M) and (c) a sequence comprising an epitope sequence from nucleocapsid phosphoprotein (N)

In some embodiments, the polypeptide comprises (a) 2, 3, 4, 5, 6, 7, 8, 9 or 10 or more epitope sequence from ORF1ab, (b) a sequence comprising an epitope sequence from membrane glycoprotein (M) and (c) a sequence comprising an epitope sequence from nucleocapsid phosphoprotein (N).

In some embodiments, the epitope sequence from ORF1ab is an epitope sequence from a non-structural protein. In some embodiments, the non-structural protein is selected from the group consisting of NSP1, NSP2, NSP3, NSP4 and combinations thereof. In some embodiments, the polypeptide comprises a sequence comprising an epitope sequence from NSP1, a sequence comprising an epitope sequence from NSP2, a sequence comprising an epitope sequence from NSP3 and a sequence comprising an epitope sequence from NSP4.

In some embodiments, the epitope sequence from ORF1ab is selected from the group consisting of YLFDESGEFKL, YLFDESGEF, FGDDTVIEV, QLMCQPILL, TTDPSFLGRY, PTDNYITTY, PSFLGRY, AEAELAKNV, KTIQPRVEK and any combination thereof.

In some embodiments, the epitope sequence from nucleocapsid glycoprotein (N) is LLLDRLNQL. In some embodiments, the epitope sequence from membrane phosphoprotein (M) is VATSRTLSY. In some embodiments, the polypeptide comprises an epitope sequence from nucleocapsid glycoprotein (N) that is LLLDRLNQL and an epitope sequence from membrane phosphoprotein (M) that is VATSRTLSY.

In some embodiments, the polypeptide comprises (a) each of the following epitope sequences from ORF1ab: YLFDESGEFKL, YLFDESGEF, FGDDTVIEV, QLMCQPILL, TTDPSFLGRY, PTDNYITTY, PSFLGRY, AEAELAKNV, KTIQPRVEK; (b) an epitope sequence from nucleocapsid glycoprotein (N) that is LLLDRLNQL; and (c) an epitope sequence from membrane phosphoprotein (M) that is VATSRTLSY.

In some embodiments, the sequence comprising an epitope sequence from ORF1ab is selected from the group consisting of the following sequences or fragments thereof:

MVTNNTFTLKVPHVGEIPVAYRKVLLKTIQPRVEKYLFDESGEFKLSEVG
PEHSLAEYYIFFASFYY; MVTNNTFTLKVPHVGEIPVAYRKVLLKTIQP
RVEKYLFDESGEFKLSEVGPEHSLAEY; APKEIIFLEGETLFGDDTVIE
VAIILASFSAST; APKEIIFLEGETLFGDDTVIEV; HTTDPSFLGRYM
SALFADDLNQLTGYHTDFSSEIIGYQLMCQPILLAEAELAKNVSLILGTV
SWNL; TTDPSFLGRYMSALFADDLNQLTGYHTDFSSEIIGYQLMCQPIL
LAEAELAKNVSLILGTVSWNL; LLSAGIFGAITDVFYKENSYKVPTDNY
ITTY; and combinations thereof.

In some embodiments, the sequence comprising an epitope sequence from membrane glycoprotein (M) is selected from the group consisting of the following sequences or fragments thereof:

ADSNGTITVEELKKLLEQWNLVIGFLFLTWICLLQFAYANRNRFLYIIKL
IFLWLLWPVTLACFVLAAVYRINWITGGIAIAMACLVGLMWLSYFIASFR
LFARTRSMWSFNPETNILLNVPLHGTILTRPLLESELVIGAVILRGHLRI
AGHHLGRCDIKDLPKEITVATSRTLSYYKLGASQRVAGDSGFAAYSRYRI
GNYKLNTDHSSSSDNIALLVQ; FAYANRNRFLYIIKLIFLWLLWPVTLA
CFVLAAVYRINWITGGIAIAMACLVGLMWLSYFIASFRLF; LGRCDIKD
LPKEITVATSRTLSYYKLGASQRVA; KLLEQWNLVIGF; NRNRFLYII
KLIFLWLLWPVTLACFVLAAVY; SELVIGAVILRGHLRIAGHHLGR;
VATSRTLSYYKLGASQRV; GLMWLSYF; and combinations
thereof.

In some embodiments, the sequence comprising an epitope sequence from nucleocapsid phosphoprotein (N) is selected from the group consisting of the following sequences or fragments thereof:

KDLSPRWYFYYLGTGPEAGLPYGANKDGIIWVATEGALNTPKDHIGTRNP
ANNAAIVLQLPQGTTLPKGFYAEGSRGGSQASSRSSSRSRNSSRNSTPGS
SRGTSPARMAGNGGDAALALLLLDRLNQLESKMSGKGQQQQGQTVTKKSA
AEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVI
LLNKHIDAYKTFPPTEPKKDKKKKADETQALPQRQKKQQTVTLLPAADLD
DFSKQLQQSMSSADSTQA;RMAGNGGDAALALLLLDRLNQLESKMSGKGQ
QQ;YKHWPQIAQFAPSASAFFGMSRIGMEVTPSGTWLTYTGAIKLDDKDP
NFKDQVILLNKHIDAYKTFP;SPARMAGNGGDAALALLLLDRLNQLESKM
SGKGQQQQGQTVTKKSAAEASKKPRQKRTATKAYNVTQAFGRRGPEQTQG
NFGDQELIRQGTDYKHWPQIAQFAPSASAFFGMSRIGMEVTPSGTWLTYT
GAIKLDDKDPNFKDQVILLNKHIDAYKTFPPTEPKKDK and
combinations thereof.

In some embodiments, the polypeptide comprises one or more linker sequences. In some embodiments, the one or more linker sequences are selected from the group consisting of GGSGGGGSGG, GGSLGGGGSG. In some embodiments, the one or more linker sequences comprise cleavage sequences. In some embodiments, the one or more cleavage sequences are selected from the group consisting of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA.

In some embodiments, the polypeptide comprises a transmembrane domain sequence. In some embodiments, the transmembrane sequence is C-terminal to the sequence comprising an epitope sequence from ORF1ab, the sequence comprising an epitope sequence from membrane glycoprotein (M) and the sequence comprising an epitope sequence from nucleocapsid phosphoprotein (N). In some embodiments, the is transmembrane sequence EQYIKWPWYIWLGFIAGLIAIVMVTIMLCCMTSCCSCLKGCCSCGSCCKFDEDDSEPVLKGVKL HYT.

In some embodiments, the polypeptide comprises an SEC sequence. In some embodiments, the SEC sequence is N-terminal to the sequence comprising an epitope sequence from ORF1ab, the sequence comprising an epitope sequence from membrane glycoprotein (M) and the sequence comprising an epitope sequence from nucleocapsid phosphoprotein (N). In some embodiments, the SEC sequence is MFVFLVLLPLVSSQCVNLT.

In some embodiments, the composition comprises the polynucleotide encoding the polypeptide. In some embodiments, the polynucleotide is an mRNA. In some embodiments, the polynucleotide comprises a codon optimized sequence for expression in a human.

In some embodiments, the polynucleotide comprises a dEarI-hAg sequence. In some embodiments, the dEarI-hAg sequence is ATTCTTCTGGTCCCCACAGACTCAGAGAGAACCC, optionally wherein each T is a U.

In some embodiments, the polynucleotide comprises a Kozak sequence. In some embodiments, the a Kozak sequences is GCCACC.

In some embodiments, the polynucleotide comprises an F element sequence. In some embodiments, the F element sequence is a 3 UTR of amino-terminal enhancer of split (AES). In some embodiments, the F element sequence is CTGGTACTGCATGCACGCAATGCTAGCTGCCCCTTTCCCGTCCTGGGTACCCCGAGTCTCCC CCGACCTCGGGTCCCAGGTATGCTCCCACCTCCACCTGCCCCACTCACCACCTCTGCTAGTTC CAGACACCTCC, optionally wherein each T is a U.

In some embodiments, the polynucleotide comprises an I element sequence. In some embodiments, the I element sequence is a 3′ UTR of mitochondrially encoded 12S rRNA (mtRNR1). In some embodiments, the I element sequence is CAAGCACGCAGCAATGCAGCTCAAAACGCTTAGCCTAGCCACACCCCCACGGGAAACAGCA GTGATTAACCTTTAGCAATAAACGAAAGTTTAACTAAGCTATACTAACCCCAGGGTTGGTCA ATTTCGTGCCAGCCACACC, optionally wherein each T is a U.

In some embodiments, the polynucleotide comprises a poly A sequence. In some embodiments, the poly A sequence is AAAAAAAAAAAAAAAAAAAAAAAAAAAAAAGCATATGACTAAAAAAAAAAAAAAAAAAA AAAAAAAAAAAAAAAAAAAAAAAAAAAAAAAAAAAAAAAAAAAAAAAAAAA, optionally wherein each T is a U.

In some embodiments, each of the epitope sequences from the ORF1ab, the membrane glycoprotein, and the nucleocapsid phosphoprotein are from 2019 SARS-CoV-2.

In some embodiments, one or more or each epitope elicits a T cell response.

In some embodiments, one or more or each epitope has been observed by mass spectrometry as being presented by an HLA molecule.

In some embodiments, the composition comprises (i) a polypeptide with at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; (ii) a polynucleotide encoding a polypeptide with at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or (iii) a polynucleotide with at least at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS Con2, RS C7n2, RS C8n2, RS C5n2full, RS Con2full, RS C7n2full, RS C8n2full, RS C7n4, RS C7n4full.

Also provided herein is a pharmaceutical composition comprising any of the compositions described herein.

Also provided herein is a pharmaceutical composition comprising: (i) a polypeptide comprising an epitope sequence of Table 1A, Table 1B, Table 1C, Table 2Ai, Table 2Aii, Table 2B and/or Table 16; (ii) a polynucleotide encoding the polypeptide; (iii) a T cell receptor (TCR) or a T cell comprising the TCR, wherein the TCR binds to the epitope sequence in complex with a corresponding HLA class I or class II molecule; (iv) an antigen presenting cell comprising (i) or (ii); or (v) an antibody or B cell comprising the antibody, wherein the antibody binds to the epitope sequence; and a pharmaceutically acceptable excipient.

In some embodiments, the epitope sequence comprises one or more or each of the following: YLFDESGEFKL, YLFDESGEF, FGDDTVIEV, LLLDRLNQL, QLMCQPILL, TTDPSFLGRY, PTDNYITTY, PSFLGRY, AEAELAKNV, VATSRTLSY and KTIQPRVEK. In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV, FGADPIHSL, NYNYLYRLF, KYIKWPWYI, KWPWYIWLGF, LPFNDGVYF, QPTESIVRF, IPFAMQMAY, YLOPRTFLL and RLQSLQTYV.

In some embodiments, the epitope sequence is from an orflab protein. In some embodiments, the epitope sequence is from an orfla protein In some embodiments, the epitope sequence is from a surface glycoprotein(S) or a shifted reading frame thereof. In some embodiments, the epitope sequence is from a nucleocapsid phosphoprotein (N). In some embodiments, the epitope sequence is from an ORF3a protein. In some embodiments, the epitope sequence is from a membrane glycoprotein (M). In some embodiments, the epitope sequence is from an ORF7a protein. In some embodiments, the epitope sequence is from an ORF8 protein. In some embodiments, the epitope sequence is from an envelope protein (E). In some embodiments, the epitope sequence is from an ORF6 protein. In some embodiments, the epitope sequence is from an ORF7b protein. In some embodiments, the epitope sequence is from an ORF10 protein. In some embodiments, the epitope sequence is from an ORF9b protein.

Also provided herein is a pharmaceutical composition comprising: a polypeptide having an amino acid sequence with at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15; or a recombinant polynucleotide encoding a polypeptide having an amino acid sequence with at least at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15.

In some embodiments, the pharmaceutical composition comprises a polypeptide with at least at least 70%, at least 75%, at least 80%, at least 85%, at least 90%, at least 95%, or 100% sequence identity to a sequence selected from the group consisting of RS C1p1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or a polynucleotide encoding a polypeptide with at least 70%, at least 75%, at least 80%, at least 85%, at least 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full. In some embodiments, the pharmaceutical composition comprises a polynucleotide with at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS C6n2, RS C7n2, RS C8n2, RS C5n2full, RS Con2full, RS C7n2full, RS C8n2full, RS C7n4 and RS C7n4full,.

In some embodiments, the polynucleotide is an mRNA.

In some embodiments, the pharmaceutical composition further comprises one or more lipid components. In some embodiments, the one or more lipids comprise a lipid nanoparticle (LNP). In some embodiments, the LNP encapsulates the recombinant polynucleotide construct.

In some embodiments, the polypeptide is synthetic. In some embodiments, the polypeptide is recombinant.

In some embodiments, the polypeptide is from 8-1000 amino acids in length.

In some embodiments, the epitope sequence binds to or is predicted to bind to an HLA class I or class II molecule with a K_{D} of 1000 nM or less. In some embodiments, the epitope sequence binds to or is predicted to bind to an HLA class I or class II molecule with a K_{D} of 500 nM or less.

In some embodiments, the epitope sequence comprises a sequence of a viral protein expressed by a virus-infected cell of a subject.

Also provided herein is a method of treating or preventing an infection by a virus or treating a respiratory disease or condition associated with an infection by a virus comprising administering to a subject in need thereof a pharmaceutical composition described herein.

In some embodiments, the virus is a coronavirus. In some embodiments, the virus is 2019 SARS-CoV 2. In some embodiments, an HLA molecule expressed by the subject is unknown at the time of administration. In some embodiments, the ability of the virus to avoid escape of recognition by an immune system of the subject is less compared to the ability of the virus to avoid escape of recognition by an immune system of a subject administered a pharmaceutical composition containing an epitope from a single protein or epitopes from fewer proteins than in a pharmaceutical composition described herein. In some embodiments, the subject express an HLA molecule encoded by an HLA allele of any one of Table 1A, Table 1B, Table 1C, Table 2Ai or Table 2Aii, Table 2B or Table 16 and the epitope sequence is an HLA allele-matched epitope sequence.

In some embodiments, the epitope sequence comprises one or more or each of the following: SAPPAQYEL, AVASKILGL, EYADVFHLY, DEFTPFDVV, VRIQPGQTF, SFRLFARTR, KFLPFQQF, VVQEGVLTA, RLDKVEAEV and FGADPIHSL.

Also provided herein is a method of treating or preventing a 2019 SARS-CoV 2 infection in a subject in need thereof, comprising administering to the subject a pharmaceutical composition described herein.

In some embodiments, the pharmaceutical composition is administered in addition to one or more therapeutics for the 2019 SARS-CoV 2 viral infection in the subject. In some embodiments, the pharmaceutical composition is administered in combination with (a) a polypeptide having an amino acid sequence of a 2019 SARS-CoV 2 spike protein or a variant or fragment thereof; (b) a recombinant polynucleotide encoding a 2019 SARS-CoV 2 spike protein or a variant or fragment thereof; or a 2019 SARS-CoV 2 spike protein pharmaceutical composition comprising (a) or (b). In some embodiments, the 2019 SARS-CoV 2 spike protein or a variant or fragment thereof is a SARS-CoV-2 spike protein or a fragment thereof.

In some embodiments, the pharmaceutical composition is administered 1-10 weeks after a first administration of the 2019 SARS-CoV 2 spike protein pharmaceutical composition. In some embodiments, the pharmaceutical composition is administered 1-6 weeks, 1-6 months or 1-2 years or later after a first administration of the 2019 SARS-CoV 2 spike protein pharmaceutical composition. In some embodiments, the pharmaceutical composition is administered on the same day or simultaneously with an administration of the 2019 SARS-CoV 2 spike protein pharmaceutical composition. In some embodiments, the pharmaceutical composition is co-formulated with the polypeptide having an amino acid sequence of a 2019 SARS-CoV 2 spike protein or a variant or fragment thereof or the recombinant polynucleotide encoding a 2019 SARS-CoV 2 spike protein or a variant or fragment thereof. In some embodiments, the pharmaceutical composition is administered before an administration of the 2019 SARS-CoV 2 spike protein pharmaceutical composition, such as 2-10 weeks before an administration of the 2019 SARS-CoV 2 spike protein pharmaceutical composition. In some embodiments, the pharmaceutical composition is administered prophylactically. In some embodiments, the pharmaceutical composition is administered once every 1, 2, 3, 4, 5, 6 or more weeks; or once every 1-7, 7-14, 14-21, 21-28, or 28-35 days; or once every 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days.

Also provided herein is a use of a composition described herein for preparing a therapeutic for treating or preventing a respiratory viral infection caused by 2019 SARS CoV-2 virus.

Also provided herein is a composition described herein or a pharmaceutical composition described herein for use as a medicament.

Also provided herein is a composition described herein or a pharmaceutical composition described herein for use in the treatment or prevention of a respiratory viral infection caused by 2019 SARS CoV-2 virus.

Provided herein is an antigenic peptide comprising an epitope sequence from Table 1A, Table 1B, Table 1C, Table 2Ai, Table 2Aii or Table 2B. Also provided herein is a polynucleotide encoding and antigenic peptide comprising an epitope sequence from Table 1A, Table 1B, Table 1C, Table 2Ai, Table 2Aii or Table 2B. The antigenic peptide and/or polynucleotide may be recombinant. The antigenic peptide and/or polynucleotide may be isolated or purified. The antigenic peptide may be synthetic or expressed from a polynucleotide.

Also provided herein is an antibody or B cell comprising an antibody that binds to an antigenic peptide comprising an epitope sequence from Table 1A, Table 1B, Table 1C, Table 2Ai, Table 2Aii or Table 2B.

Also provided herein is a T cell receptor (TCR) or T cell comprising a TCR that binds an epitope sequence from Table 1A or Table 1B in complex with a corresponding MHC class I molecule according to Table 1A or Table 1B. For example, the TCR can bind to an epitope sequence from column 2 (set 1) of Table 1A in complex with a corresponding MHC class I molecule from column 3 (set 1) in the same row of Table 1A. For example, the TCR can bind to an epitope sequence from column 4 (set 2) of Table 1A in complex with a corresponding MHC class I molecule from column 5 (set 2) in the same row of Table 1A. For example, the TCR can bind to an epitope sequence from column 6 (set 3) of Table 1A in complex with a corresponding MHC class I molecule from column 7 (set 3) in the same row of Table 1A. For example, the TCR can bind to an epitope sequence from column 2 (set 1) of Table 1B in complex with a corresponding MHC class I molecule from column 3 (set 1) in the same row of Table 1B. For example, the TCR can bind to an epitope sequence from column 4 (set 2) of Table 1B in complex with a corresponding MHC class I molecule from column 5 (set 2) in the same row of Table 1B.

Also provided herein is a T cell receptor (TCR) or T cell comprising a TCR that binds to an epitope sequence from Table 2Ai or Table 2Aii in complex with a corresponding MHC class II molecule according to Table 2Ai or Table 2Aii. For example, the TCR can bind to an epitope sequence from column 2 (set 1) of Table 2Ai in complex with a corresponding MHC class II molecule from column 3 (set 1) in the same row of Table 2Ai. For example, the TCR can bind to an epitope sequence from column 4 (set 2) of Table 2Ai in complex with a corresponding MHC class II molecule from column 5 (set 2) in the same row of Table 2Ai. Likewise, a TCR can bind to an epitope sequence from the left column of Table 2Aii in complex with a corresponding MHC class II molecule from the right column of Table 2Aii.

Provided herein is a method of treating or preventing viral infection in a subject in need thereof comprising administering to the subject an antigenic peptide comprising an epitope sequence from Table 1A, Table 1B, Table 1C, Table 2Ai, Table 2Aii or Table 2B. Also provided herein is a method of treating or preventing viral infection in a subject in need thereof comprising administering to the subject a polynucleotide encoding and antigenic peptide comprising an epitope sequence from Table 1A, Table 1B, Table 1C, Table 2Ai, Table 2Aii or Table 2B.

Also provided herein is a method of treating or preventing a viral infection in a subject in need thereof comprising administering to the subject an antibody or B cell comprising an antibody that binds to an antigenic peptide comprising an epitope sequence from Table 1A, Table 1B, Table 1C, Table 2Ai, Table 2Aii or Table 2B.

Also provided herein is a method of treating or preventing viral infection in a subject in need thereof comprising administering to the subject a T cell receptor (TCR) or T cell comprising a TCR that that binds an epitope sequence from Table 1A or Table 1B in complex with a corresponding MHC class I molecule according to Table 1A or Table 1B.

For example, the method can comprise administering to the subject a TCR or T cell comprising a TCR that can bind to an epitope sequence from column 2 (set 1) of Table 1A in complex with a corresponding MHC class I molecule from column 3 (set 1) in the same row of Table 1A. For example, the method can comprise administering to a TCR or T cell comprising a TCR that can bind to an epitope sequence from column 2 (set 1) of Table 1A in complex with a corresponding MHC class I molecule from column 3 (set 1) in the same row of Table 1A to a subject that expresses the corresponding MHC class I molecule from column 3 (set 1). For example, the method can comprise administering to the subject a TCR or T cell comprising a TCR that can bind to an epitope sequence from column 4 (set 2) of Table 1A in complex with a corresponding MHC class I molecule from column 5 (set 2) in the same row of Table 1A. For example, the method can comprise administering to a TCR or T cell comprising a TCR that can bind to an epitope sequence from column 4 (set 2) of Table 1A in complex with a corresponding MHC class I molecule from column 5 (set 2) in the same row of Table 1A to a subject that expresses the corresponding MHC class I molecule from column 5 (set 2). For example, the method can comprise administering to the subject a TCR or T cell comprising a TCR that can bind to an epitope sequence from column 6 (set 3) of Table 1A in complex with a corresponding MHC class I molecule from column 7 (set 3) in the same row of Table 1A. For example, the method can comprise administering to a TCR or T cell comprising a TCR that can bind to an epitope sequence from column 6 (set 3) of Table 1A in complex with a corresponding MHC class I molecule from column 7 (set 3) in the same row of Table 1A to a subject that expresses the corresponding MHC class I molecule from column 7 (set 3).

For example, the method can comprise administering to the subject a TCR or T cell comprising a TCR that can bind to an epitope sequence from column 2 (set 1) of Table 1B in complex with a corresponding MHC class I molecule from column 3 (set 1) in the same row of Table 1B. For example, the method can comprise administering to a TCR or T cell comprising a TCR that can bind to an epitope sequence from column 2 (set 1) of Table 1B in complex with a corresponding MHC class I molecule from column 3 (set 1) in the same row of Table 1B to a subject that expresses the corresponding MHC class I molecule from column 3 (set 1). For example, the method can comprise administering to the subject a TCR or T cell comprising a TCR that can bind to an epitope sequence from column 4 (set 2) of Table 1B in complex with a corresponding MHC class I molecule from column 5 (set 2) in the same row of Table 1B. For example, the method can comprise administering to a TCR or T cell comprising a TCR that can bind to an epitope sequence from column 4 (set 2) of Table 1B in complex with a corresponding MHC class I molecule from column 5 (set 2) in the same row of Table 1B to a subject that expresses the corresponding MHC class I molecule from column 5 (set 2).

For example, the method can comprise administering to the subject a TCR or T cell comprising a TCR that can bind to an epitope sequence from column 2 (set 1) of Table 2Ai in complex with a corresponding MHC class II molecule from column 3 (set 1) in the same row of Table 2Ai. For example, the method can comprise administering to a TCR or T cell comprising a TCR that can bind to an epitope sequence from column 2 (set 1) of Table 2Ai in complex with a corresponding MHC class II molecule from column 3 (set 1) in the same row of Table 2Ai to a subject that expresses the corresponding MHC class II molecule from column 3 (set 1). For example, the method can comprise administering to the subject a TCR or T cell comprising a TCR that can bind to an epitope sequence from column 4 (set 2) of Table 2Ai in complex with a corresponding MHC class II molecule from column 5 (set 2) in the same row of Table 2Ai. For example, the method can comprise administering to a TCR or T cell comprising a TCR that can bind to an epitope sequence from column 4 (set 2) of Table 2Ai in complex with a corresponding MHC class II molecule from column 5 (set 2) in the same row of Table 2Ai to a subject that expresses the corresponding MHC class II molecule from column 5 (set 2). For example, the method can comprise administering to the subject a TCR or T cell comprising a TCR that can bind to an epitope sequence from the left column of Table 2Aii in complex with a corresponding MHC class II molecule from the right column in the same row of Table 2Aii.

In one embodiment, the antigenic peptide is a viral antigen. In another embodiment, the antigenic peptide is a non-mutated overexpressed antigen. In some embodiments, the viral antigen is derived from publicly disclosed information on the viral genetic information. In some embodiments, the viral antigen is derived from analysis of the viral genome to predict suitable epitopes for T cell activation. In some embodiments, the viral antigen is derived from analysis of the sequence of the viral genome in a MHC-peptide presentation prediction algorithm implemented in a computer processor. In some embodiments, the viral antigen is derived from analysis of the viral sequences in an MHC-peptide presentation prediction algorithm implemented in a computer processor that has been trained by a machine learning software, which predicts the likelihood of binding and presentation of an epitope by an MHC class I or an MHC class II antigen. In some embodiments, the MHC-peptide presentation predictor is neonmhc2.

In some embodiments, the MHC-peptide presentation prediction algorithm or MHC-peptide presentation predictor is NetMHCpan or NetMHCIIpan and in addition, further analyzed in MHC-peptide presentation predictor NetMHCpan or NetMHCIIpan for comparison. In some embodiments, a skilled artisan may use hidden markov model approach for MHC-peptide presentation prediction. In some embodiments, the peptide prediction model MARIA may be utilized. In some embodiments, the MHC-peptide presentation prediction algorithm or MHC-peptide presentation predictor used is not NetMHCpan or NetMHCIIpan. In some embodiments, the viral sequences are analyzed in MHC-peptide presentation prediction algorithm implemented in a computer processor where the MHC-peptide presentation predictor is neonmhc 1 or neonmhc2, that refer respectively to class I and class II binding prediction. In some embodiments, the MHC-peptide presentation predictor model is RECON, which offers high quality MHC-peptide presentation prediction based on expression, processing and binding capabilities.

In one aspect, provided herein is a method of treating a viral disease in a subject caused by a coronavirus, comprising: administering to the subject a composition comprising one or more viral peptide antigens, wherein the viral peptide antigens are predicted to bind to an MHC class I or an MHC class II peptide of the subject, and are predicted to be presented by an antigen presenting cell to a T cell of the subject such that an antiviral response is initiated in the subject. In some embodiments, the viral antigen is derived from analysis of the sequence of the viral genome in a MHC-peptide presentation prediction algorithm implemented in a computer processor. In some embodiments, the viral antigen is derived from analysis of the viral sequences in an MHC-peptide presentation prediction algorithm implemented in a computer processor that has been trained by a machine learning software, which predicts the likelihood of binding and presentation of an epitope by an MHC class I or an MHC class II antigen. In some embodiments, the MHC-peptide presentation predictor is neonmhc2. In some embodiments, the method further comprises analyzing nucleic acid sequence derived from viral genome in an MHC-peptide presentation prediction model, comprising an algorithm implemented in a computer processor that has been trained by a machine learning software, wherein the MHC-peptide presentation prediction model predicts the likelihood of binding and presentation of an epitope encoded by the viral genome by an MHC class I or an MHC class II antigen. In some embodiments, the method further comprises analyzing a biological sample from a subject for identification of the MHC class I and MHC class II repertoire, wherein the analyzing comprises analyzing by genome or whole exome sequencing or by analysis of proteins encoded by an HLA gene. In some embodiments, the method further comprises matching the epitopes predicted by the MHC-peptide presentation prediction model that have a high affinity for an MHC class I or an MHC class II peptide encoded by an HLA gene of the subject, and selecting one or more peptides that are predicted to bind an MHC peptide encoded by an HLA gene of the subject with a high affinity ranked by the MHC-peptide presentation prediction model. In some embodiments, the one or more peptides that are selected have been predicted to bind an MHC peptide encoded by an HLA gene of the subject with an affinity of at least 1000 nM. In some embodiments, the one or more peptides that are selected have been predicted to bind an MHC class I peptide encoded by an HLA gene of the subject with an affinity of at least 500 nM. In some embodiments, the one or more peptides that are selected have been predicted to bind an MHC class II peptide encoded by an HLA gene of the subject with an affinity of at least 1000 nM.

In some embodiments, the MHC-peptide presentation prediction model is programmed to provide a ranking order in decreasing order of a likelihood for a particular epitope or antigenic peptide to bind to an HLA allele that would present the peptide to a T cell receptor. In some embodiments, epitope sequences that have the highest likelihood of binding and being presented by an HLA are selected for preparing a therapeutic. In some embodiments, the selection of the HLA may be restricted by HLA expressed in a subject. In some embodiments, the selection of the HLA may be based on the prevalence (e.g., higher prevalence) of the allele in a population. In some embodiments the epitopes may be selected for preparing a therapeutic based on the higher likelihood for the peptide (epitope) of binding to and being presented by an HLA allele, e.g., an HLA allele of interest. In some embodiments, this % rank value may be determined by evaluating the percentile in which a query peptide scores for a specific allele compared to a fixed set of reference peptides (with a different set of reference peptides for class I and class II). In some embodiments the top 10% of the epitopes that have the highest likelihood of binding to an HLA allele may be selected. In some embodiments the top 2% of the epitopes that have the highest likelihood of binding to an HLA allele may be selected. In some embodiments the top 5% of the epitopes that have the highest likelihood of binding to an HLA allele may be selected. In some embodiments the top 8% of the epitopes that have the highest likelihood of binding to an HLA allele may be selected. In some embodiments the top 1% of the epitopes that have the highest likelihood of binding to an HLA allele may be selected. In some embodiments the top 0.5% of the epitopes that have the highest likelihood of binding to an HLA allele may be selected. In some embodiments the top 0.1% of the epitopes that have the highest likelihood of binding to an HLA allele may be selected. In some embodiments the top 0.01% of the epitopes that have the highest likelihood of binding to an HLA allele may be selected. In some embodiments the selection of the cut off may be dependent on the availability and number of epitopes predicted to have a high likelihood of binding to an HLA allele as determined by the prediction model.

In some embodiments, the subject may be infected by the virus. In some embodiments, the subject may be at risk of infection by the virus. In some embodiments, the virus is a coronavirus. In some embodiments, the coronavirus is selected from a SARS virus, a MERS coronavirus or a 2019 SARS COV-2 virus. In some embodiments, the one or more viral peptide antigen comprises a peptide comprising at least 8 contiguous amino acids of a sequence in Table 1A, Table 1B, Table 1C, Table 2Ai, Table 2Aii, Table 2B, Table 9, Table 10, Table 11, Table 12, Table 14A, Table 14B, Table 15 or Table 16. In some embodiments, the one or more viral peptide antigen comprises a peptide comprising at least 7 contiguous amino acids of a sequence in Table 1A, Table 1B, Table 1C, Table 2Ai, Table 2Aii, Table 2B, Table 9, Table 10, Table 11, Table 12, Table 14A, Table 14B, Table 15 or Table 16. In some embodiments, the one or more viral peptide antigen comprises a peptide comprising at least 6 contiguous amino acids of a sequence in Table 1A, Table 1B, Table 1C, Table 2Ai, Table 2Aii, Table 2B, Table 9, Table 10, Table 11, Table 12, Table 14A, Table 14B, Table 15 or Table 16.

In one embodiment, the antigenic peptide is between about 5 to about 50 amino acids in length. In another embodiment, the antigenic peptide is between about 15 to about 35 amino acids in length. In another embodiment, the antigenic peptide is about 15 amino acids or less in length. In another embodiment, the antigenic peptide is between about 8 and about 11 amino acids in length. In another embodiment, the antigenic peptide is 9 or 10 amino acids in length. In one embodiment, the antigenic peptide binds major histocompatibility complex (MHC) class I. In another embodiment, the antigenic peptide binds MHC class I with a binding affinity of less than about 500 nM.

In one embodiment, the antigenic peptide is about 30 amino acids or less in length. In another embodiment, the antigenic peptide is between about 6 and about 25 amino acids in length. In another embodiment, the antigenic peptide is between about 15 and about 24 amino acids in length. In another embodiment, the antigenic peptide is between about 9 and about 15 amino acids in length. In one embodiment, the antigenic peptide binds MHC class II. In another embodiment, the antigenic peptide binds MHC class II with a binding affinity of less than about 1000 nM.

In one embodiment, the antigenic peptide further comprises flanking amino acids. In another embodiment, the flanking amino acids are not native flanking amino acids. In one embodiment, the antigenic peptide is linked to at least a second antigenic peptide. In another embodiment, the peptides are linked using a poly-glycine or poly-serine linker. In another embodiment, the second antigenic peptide binds MHC class I or class II with a binding affinity of less than about 1000 nM. In another embodiment, the second antigenic peptide binds MHC class I or class II with a binding affinity of less than about 500 nM. In another embodiment, both of the epitopes bind to human leukocyte antigen (HLA)-A, -B, -C, -DP, -DQ, or -DR. In another embodiment, the antigenic peptide binds a class I HLA and the second antigenic peptide binds a class II HLA. In another embodiment, the antigenic peptide binds a class II HLA and the second antigenic peptide binds a class I HLA.

In one embodiment, the antigenic peptide further comprises modifications which increase in vivo half-life, cellular targeting, antigen uptake, antigen processing, MHC affinity, MHC stability, or antigen presentation. In another embodiment, the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids, for example, synthetic amino acids, or f-moc amino acids, D-amino acids N-methyl amino acids. In one embodiment, the cells that are targeted are antigen presenting cells. In another embodiment, the antigen presenting cells are dendritic cells. In another embodiment, the dendritic cells are targeted using DEC205, XCR1, CD197, CD80, CD86, CD123, CD209, CD273, CD283, CD289, CD184, CD85h, CD85j, CD85k, CD85d, CD85g, CD85a, CD141, CD11 c, CD83, TSLP receptor, or CD1a marker. In another embodiment, the dendritic cells are targeted using the CD141, DEC205, or XCR1 marker.

In one embodiment, provided herein is an in vivo delivery system comprising an antigenic peptide described herein. In another embodiment, the delivery system includes cell-penetrating peptides, nanoparticulate encapsulation, virus like particles, or liposomes. In another embodiment, the cell-penetrating peptide is TAT peptide, herpes simplex virus VP22, transportan, or Antp.

In one embodiment, provided herein is a cell comprising an antigenic peptide described herein. In another embodiment, the cell is an antigen presenting cell. In another embodiment, the cell is a dendritic cell.

In one embodiment, provided herein is a composition comprising an antigenic peptide described herein. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the antigenic peptides comprising an epitope of Table 1A. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the antigenic peptides comprising an epitope of Table 1B. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the antigenic peptides comprising an epitope of Table 2B. In another embodiment, the composition comprises between 2 and 20 antigenic peptides. In another embodiment, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, at least 26, at least 27, at least 28, at least 29, or at least 30 additional antigenic peptides. In another embodiment, the composition comprises between about 4 and about 20 additional antigenic peptides. In another embodiment, the additional antigenic peptide is specific for coronavirus.

In one embodiment, provided herein is a polynucleotide encoding the antigenic peptide described herein. In another embodiment, the polynucleotide is RNA, optionally a self-amplifying RNA. In some embodiments the polynucleotide is DNA. In another embodiment, the RNA is modified to increase stability, increase cellular targeting, increase translation efficiency, adjuvanticity, cytosol accessibility, and/or decrease cytotoxicity. In another embodiment, the modification is conjugation to a carrier protein, conjugation to a ligand, conjugation to an antibody, codon optimization, increased GC-content, incorporation of modified nucleosides, incorporation of 5′-cap or cap analog, and/or incorporation of a poly-A sequence e.g., an unmasked poly-A sequence, or a disrupted poly-A sequence in which two segments of contiguous A sequences linked by a linker.

In one embodiment, provided herein is a cell comprising a polynucleotide described herein.

In one embodiment, provided herein is a vector comprising a polynucleotide described herein. In another embodiment, the polynucleotide is operably linked to a promoter. In another embodiment, the vector is a self-amplifying RNA replicon, plasmid, phage, transposon, cosmid, virus, or virion. In another embodiment, the vector is an adeno-associated virus, herpesvirus, lentivirus, or pseudotypes thereof

In one embodiment, provided herein is an in vivo delivery system comprising an polynucleotide described herein. In another embodiment, the delivery system includes spherical nucleic acids, viruses, virus-like particles, plasmids, bacterial plasmids, or nanoparticles.

In one embodiment, provided herein is a cell comprising a vector or delivery system described herein. In another embodiment, the cell is an antigen presenting cell. In another embodiment, the cell is a dendritic cell. In another embodiment, the cell is an immature dendritic cell.

In some embodiments, provided herein is a composition comprising at least one polynucleotide described herein. In some embodiments, provided herein is a composition comprising one or more antigenic peptides described herein in combination with one or more 2019 SARS CoV-2 vaccines (e.g., mRNA-based vaccines, DNA-based vaccines, AAV-based vaccines, protein-based vaccines). In some embodiments, provided herein is a composition comprising one or more polynucleotides encoding at least one antigenic peptide described herein in combination with one or more 2019 SARS CoV-2 vaccines (e.g., mRNA-based vaccines, DNA-based vaccines, AAV-based vaccines, protein-based vaccines). In some embodiments, provided herein is a single polynucleotide encoding more than one antigenic peptide as described herein. In some embodiments, provided herein is a single polynucleotide encoding (i) at least one antigenic peptide as described herein and (ii) a 2019 SARS CoV-2 protein (e.g., S protein) and/or immunogenic fragments thereof (e.g., receptor binding domain (RBD) of S protein). In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the polynucleotides. In another embodiment, the composition comprises between about 2 and about 20 polynucleotides. In another embodiment, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 additional antigenic polynucleotides encoding for additional antigenic peptides. In another embodiment, the composition comprises between about 4 and about 20 additional antigenic polynucleotides. In another embodiment, the polynucleotides and the additional antigenic polynucleotides are linked. In another embodiment, the polynucleotides are linked using nucleic acids that encode a poly-glycine or poly-serine linker.

In one embodiment, provided herein is a T cell receptor (TCR) capable of binding at least one antigenic peptide described herein. In another embodiment, the TCR is capable of binding the antigenic peptide in the context of MHC class I or class II.

In one embodiment, provided herein is a chimeric antigen receptor comprising: (i) a T cell activation molecule; (ii) a transmembrane region; and (iii) an antigen recognition moiety capable of binding an antigenic peptide described herein. In another embodiment, CD3-zeta is the T cell activation molecule. In another embodiment, the chimeric antigen receptor further comprises at least one costimulatory signaling domain. In another embodiment, the signaling domain is CD28, 4-1BB, ICOS, OX40, ITAM, or Fc epsilon RI-gamma. In another embodiment, the antigen recognition moiety is capable of binding the antigenic peptide in the context of MHC class I or class II. In another embodiment, the chimeric antigen receptor comprises the CD3-zeta, CD28, CTLA-4, ICOS, BTLA, KIR, LAG3, CD137, OX40, CD27, CD40L, Tim-3, A2aR, or PD-1 transmembrane region.

In one embodiment, provided herein is a T cell comprising the T cell receptor or chimeric antigen receptor described herein. In one embodiment, the T cell is a helper or cytotoxic T cell.

In one embodiment, provided herein is a nucleic acid comprising a promoter operably linked to a polynucleotide encoding a T cell receptor described herein. In another embodiment, the TCR is capable of binding the at least one antigenic peptide in the context of major histocompatibility complex (MHC) class I or class II. In one embodiment, the nucleic acid comprises a promoter operably linked to a polynucleotide encoding a chimeric antigen receptor described herein. In another embodiment, the antigen recognition moiety is capable of binding the at least one antigenic peptide in the context of major histocompatibility complex (MHC) class I or class II.

In one embodiment, provided herein is an antibody capable of binding a peptide comprising an epitope of Table 1A, Table 1B, Table 1C, Table 2Ai, Table 2Aii, Table 2B, Table 9, Table 10, Table 11, Table 12, Table 14A, Table 14B, Table 15 or Table 16. In one embodiment, provided herein is an antibody capable of binding a peptide comprising an epitope of Table 1B. In one embodiment, provided herein is an antibody capable of binding a peptide comprising an epitope of Table 2Ai or Table 2Aii.

In one embodiment, provided herein is a modified cell transfected or transduced with a nucleic acid described herein. In one embodiment, the modified cell is a T cell, tumor infiltrating lymphocyte, NK-T cell, TCR-expressing cell, CD4+ T cell, CD8+ T cell, or NK cell.

In one embodiment, provided herein is a composition comprising a T cell receptor or chimeric antigen receptor described herein. In another embodiment, a composition comprises autologous patient T cells containing a T cell receptor or chimeric antigen receptor described herein. In another embodiment, the composition further comprises an immune checkpoint inhibitor. In another embodiment, the composition further comprises at least two immune checkpoint inhibitors. In another embodiment, each of the immune checkpoint inhibitors inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CLIK 1, CHK2, A2aR, and B-7 family ligands or a combination thereof. In another embodiment, each of the immune checkpoint inhibitors interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

In one embodiment, the composition further comprises an immune modulator or adjuvant. In another embodiment, the immune modulator is a co-stimulatory ligand, a TNF ligand, an Ig superfamily ligand, CD28, CD80, CD86, ICOS, CD40L, OX40, CD27, GITR, CD30, DR3, CD69, or 4-1BB. In another embodiment, the immune modulator is at least one an infected cell extract. In another embodiment, the infected cell is autologous to the subject in need of the composition. In another embodiment, the infected cell has undergone lysis or been exposed to UV radiation. In another embodiment, the composition further comprises an adjuvant. In another embodiment, the adjuvant is selected from the group consisting of: Poly(I:C), Poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel®. vector system, PLG microparticles, resiquimod, SRL172, virosomes and other virus-like particles, YF-17D, VEGF trap, R848, beta-glucan, Pam3Cys, Pam3CSK4, acrylic or methacrylic polymers, copolymers of maleic anhydride, and QS21 stimulon. In another embodiment, the adjuvant induces a humoral when administered to a subject. In another embodiment, the adjuvant induces a T helper cell type 1 when administered to a subject.

In one embodiment, provided herein is a method of inhibiting infection by a virus by administering to a subject who has a likelihood of getting infected by the virus, a vaccine composition comprising one or more peptides comprising at least 8 contiguous amino acids from the epitopes defined in Table 1A, Table 1B, Table 1C, Table 2Ai, Table 2Aii or Table 2B, comprising contacting a cell with a peptide, polynucleotide, delivery system, vector, composition, antibody, or cells of the present disclosure.

In one embodiment, provided herein is a method of treating a viral infection specifically, a coronaviral infection, for example a 2019 SARS CoV-2 infection by enhancing, or prolonging an antiviral response in a subject in need thereof comprising administering to the subject the peptide, polynucleotide, vector, composition, antibody, or cells described herein.

In one embodiment, the subject is a human. In another embodiment, the subject has a viral infection. In one embodiment, the subject is infected by a respiratory virus, such as an acute respiratory virus, for example, a SARS-like virus or a MERS or MERS-like virus, or more specifically, a coronavirus of the 2019 SARS CoV-2 strain. In some embodiments, the subject is infected with a 2019 SARS CoV-2 coronavirus. In some embodiments, the subject has been detectably infected with the 2019 SARS CoV-2 coronavirus. In some embodiments, the subject is asymptomatic. In some embodiments, the subject is symptomatic. In some embodiments, the subject is not detected to have been infected by a 2019 SARS CoV-2 virus or a related virus, but the subject is in close proximity of an infected person, in an infected area or otherwise at risk of infection.

In one embodiment of the method, a peptide is administered. In another embodiment, the administration is systemic. In another embodiment of the method, a polynucleotide, optionally RNA, is administered. In one embodiment, the polynucleotide is administered parenterally. In one embodiment, the polynucleotide is administered intravenously. In another embodiment, the polynucleotide is administered intradermally or intramuscularly, or subcutaneously. In one embodiment, the polynucleotide is administered intramuscularly. In one embodiment of the method, a cell is administered. In another embodiment, the cell is a T cell or dendritic cell. In another embodiment, the peptide or polynucleotide comprises an antigen presenting cell targeting moiety.

In one embodiment, the peptide, polynucleotide, vector, composition, or cells is administered prior to administering concurrent with another therapy, such as another antiviral therapy. In another embodiment, the peptide, polynucleotide, vector, composition, or cells is administered before or after the another antiviral therapy. In another embodiment, administration of the another antiviral therapy is continued throughout antigen peptide, polynucleotide, vector, composition, or cell therapy.

In one embodiment of the method, an additional agent is administered. In another embodiment, the agent is a chemotherapeutic agent, an immunomodulatory drug, an immune metabolism modifying drug, a targeted therapy, radiation an anti-angiogenesis agent, or an agent that reduces immune-suppression. In another embodiment, the administration of a pharmaceutical composition described herein elicits or promotes a CD4+ T cell immune response. In another embodiment, the administration of a pharmaceutical composition described herein elicits or promotes a CD4+ T cell immune response and a CD8+ T cell immune response.

In another embodiment, the patient received a chemotherapeutic agent, an immunomodulatory drug, an immune metabolism modifying drug, targeted therapy or radiation prior to and/or during receipt of the antigen peptide or nucleic acid vaccine. In another embodiment, the autologous T cells are obtained from a patient that has already received at least one round of T cell therapy containing an antigen. In another embodiment, the method further comprises adoptive T cell therapy. In another embodiment, the adoptive T cell therapy comprises autologous T cells. In another embodiment, the autologous T cells are targeted against viral antigens. In another embodiment, the adoptive T cell therapy further comprises allogenic T cells. In another embodiment, the allogenic T cells are targeted against viral antigens.

In one embodiment, provided herein is a method for evaluating the efficacy of treatment comprising: (i) measuring the number or concentration of target cells in a first sample obtained from the subject before administering the modified cell, (ii) measuring the number or concentration of target cells in a second sample obtained from the subject after administration of the modified cell, and (iii) determining an increase or decrease of the number or concentration of target cells in the second sample compared to the number or concentration of target cells in the first sample. In another embodiment, the treatment efficacy is determined by monitoring a clinical outcome; an increase, enhancement or prolongation of antiviral activity by T cells; an increase in the number of antiviral T cells or activated T cells as compared with the number prior to treatment; B cell activity; CD4 T cell activity; or a combination thereof. In another embodiment, the treatment efficacy is determined by monitoring a biomarker. In another embodiment, the treatment effect is predicted by presence of T cells or by presence of a gene signature indicating T cell inflammation or a combination thereof.

Provided herein a pharmaceutical composition comprising: one or more polypeptides having an amino acid sequence of any one of the sequences depicted in column 2 of Table 11 and 12; or one or more recombinant polynucleotide constructs each encoding a polypeptide having an amino acid sequence of any one of the sequences depicted in column 2 of Table 11 and 12.

In some embodiments, the one or more polypeptides comprises at least 2, 3, 4, 5, 6, 7 or 8 different polypeptides having an amino acid sequence of any one of the sequences depicted in column 2 of Table 11 and 12; or wherein the one or more recombinant polynucleotide constructs comprises at least 2, 3, 4, 5, 6, 7 or 8 recombinant polynucleotide constructs each encoding a different polypeptide having an amino acid sequence of any one of the sequences depicted in column 2 of Table 11 and 12. In some embodiments, the pharmaceutical composition comprises at least 8 recombinant polynucleotide strings. In some embodiments, the one or more recombinant polynucleotide strings encoding a plurality of coronavirus peptide antigens, comprises a sequence selected from a group of sequences depicted in SEQ ID RS C1n, RS C2n, RS C3n, RSC4n, RS C5n, RS Con, RS C7n, and RS C8n, or a sequence that is at least 70%, at least 75%, at least 80%, at least 85%, at least 90%, at least 95%, or 100% sequence identity to any one of the above. In some embodiments, the recombinant polynucleotide construct comprises an mRNA. In some embodiments, the recombinant polynucleotide construct is an mRNA. In some embodiments, the pharmaceutical composition further comprises one or more lipid components. In some embodiments, the one or more lipids comprise a lipid nanoparticle (LNP). In some embodiments, the LNP encapsulates the recombinant polynucleotide construct. In some embodiments, the pharmaceutical composition is administered to a subject in need thereof.

Provided herein is a method of treating COVID in a subject in need thereof, comprising administering to the subject a pharmaceutical composition described above. In some embodiments, the pharmaceutical composition is administered in addition to one or more therapeutic for COVID. In some embodiments, the pharmaceutical composition is administered in combination with one or more polypeptides having an amino acid sequence of a 2019 SARS CoV-2 spike protein or fragment thereof; or one or more recombinant polynucleotide constructs encoding a 2019 SARS CoV-2 spike protein or fragment thereof. In some embodiments, the 2019 SARS CoV-2 spike protein or fragment thereof is a SARS-CoV-2 spike protein or a fragment thereof. In some embodiments, the pharmaceutical composition is administered 2-10 weeks after a first administration of the 2019 SARS CoV-2 spike protein or fragment thereof. In some embodiments, the pharmaceutical composition is administered 1-6 months after a first administration of the 2019 SARS CoV-2 spike protein or fragment thereof. In some embodiments, the pharmaceutical composition is administered simultaneously with an administration of the 2019 SARS CoV-2 spike protein or fragment thereof. In some embodiments, the pharmaceutical composition is administered 2-10 weeks before an administration of the 2019 SARS CoV-2 spike protein or fragment thereof. In some embodiments, the pharmaceutical composition is administered 2-10 weeks after the first administration of vaccine comprising a SARS-CoV-2 spike protein or polynucleotide encoding the same. In some embodiments, the pharmaceutical composition is administered 1-6 months after the first administration of a SARS-CoV-2 spike protein or polynucleotide encoding the same. In some embodiments, the pharmaceutical composition is administered simultaneously with the administration of a SARS-CoV-2 spike protein or polynucleotide encoding the same. In some embodiments, the pharmaceutical composition is administered prophylactically. In some embodiments, the pharmaceutical composition is administered once every 1, 2, 3, 4, 5, 6 or more weeks.

Provided herein is an use of any one of the compositions described herein for preparing a therapeutic for treating or preventing a respiratory viral infection caused by 2019 SARS CoV-2 virus.

Where aspects or embodiments of the present disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the embodiments of the present disclosure.

### BRIEF DESCRIPTION OF THE DRAWINGS

FIG. 1A depicts an exemplary flow diagram of a method to identify peptides most relevant to the generation of CD8+ T cell responses against the viral epitopes described herein.

FIG. 1B shows a graphic representation of the SARS-CoV 2 genome.

FIG. 2 depicts exemplary graphs of results obtained using a T cell epitope prediction algorithm applied to class I peptide-MHC allele pairs in a validation dataset and comparison of the computed percent-ranks of these pairs with reported MHC-binding assay results. The percent-ranks of peptide-MHC allele pairs which had a binary “Positive” result in the MHC-binding assay were significantly lower than pairs with a “Negative” result. In the more granular positive results, stronger assay results (low<intermediate<high) were associated with significantly lower percent-ranks.

FIG. 3 depicts experimental validation of HLA-A02:01 predicted epitopes from 2019 SARS COV-2 in human T cell induction assays. 23 peptides that were predicted to be high binders to HLA-A02:01 (see Table 4 of Example 8) were synthesized and assayed in T cell inductions using PBMCs from three human donors. Epitopes were considered to be immunogenic if at least one donor raised a T cell response to the peptide as determined by pMHC multimer technology. Representative flow cytometry plots of pMHC staining using peptides from Table 4 of Example 8 are shown. Multimer positive populations are circled, with the frequency of multimer positive CD8+ T cells shown in the upper right-hand corner of each plot.

FIG. 4A depicts exemplary graphs of cumulative USA population coverage of HLA alleles for the indicated peptides predicted to be MHC class I epitopes (left) and the cumulative USA population coverage of HLA alleles for 25mer peptides predicted to be MHC class II epitopes (right).

FIG. 4B depicts a small number of predicted multi-allele binding epitopes from individual 2019 SARS-CoV-2 proteins (alternatively termed 2019-CoV-2 proteins) can achieve broad population coverage. The upper panel shows cumulative HLA-I coverage for USA, EUR, and API populations versus the number of included prioritized HLA-I epitopes for M, N, and S proteins, respectively. Peptide sequences corresponding to the upper panel are shown in Table 6. The lower panel shows cumulative HLA-II coverage for each population versus the number of included prioritized HLA-II 25mers for M, N, and S proteins, respectively. Peptide sequences corresponding to the lower panel are shown in Table 7.

FIG. 5 depicts results from analysis of publicly available proteomic datasets showing relative 2019 SARS CoV-2 protein expression levels that can be leveraged to prioritize potential vaccine targets. Three datasets examining the proteomic response to 2019 SARS CoV-2 infection (alternatively termed 2019 SARS CoV-2 infection) were re-analyzed and protein abundance was estimated by spectral counts normalized to protein length. Any annotated ORF not shown in the figure was not detected in these proteomic studies. Across all three studies, the nucleocapsid protein is the most abundant protein during 2019 SARS CoV-2 infection.

FIG. 6A depicts a graphical representation of a string construct described as group 1, also described in Tables 9 and 11.

FIG. 6B provides a detailed and expanded view of the constructs in FIG. 6A.

FIG. 7A depicts a graphical representation of a string construct described as group 2, also described in Tables 10 and 12.

FIG. 7B provides a detailed and expanded view of the constructs in FIG. 7A.

FIG. 8Ai-8Aii show characterization of BNT mRNA vaccine-induced T cells on a single epitope level. Included data shows epitope responsive T cells for the indicated epitopes in three different participants. The vaccine comprises mRNA encoding a SARS-CoV-2 spike protein of 2019 SARS COV-2 encapsulated in a lipid nanoparticle.

FIG. 8B shows multimer positive CD8+ cells analysed by flow cytometry for cell surface markers, CCR7, CD45RA, CD3, PD-1, CD38, HLA-DR, CD28 and CD27.

FIG. 8C shows a mRNA vaccine including the spike proteins S1 and S2, with indicated epitope regions that can bind to specific MHC molecules indicated by the solid shapes along the length, corresponding HLA allele to which it binds is indicated below.

FIG. 8D shows time course of T cell responses after vaccination of patients with Spike protein mRNA vaccines at different doses (10, 20 and 30 micrograms as indicated). Upper panel shows CD4+ T cell responses, indicated by IFN-g expression using ELISPOT assay. Lower panel shows CD8+ T cell responses, indicated by IFN-g expression using ELISPOT assay. CEF and CEFT are controls CMV, EBV and influenza pools.

FIG. 8E shows time course of CD4+ T cells and CD8+ T cell responses in older adult population who are administered Spike protein mRNA vaccine (30 microgram each).

FIG. 9 shows design of vaccine strings comprising ORF-1ab epitopes, with specific use of MS-based HLA-I cleavage predictor information in ordering the epitopes. The design utilizes minimum number of linker sequences.

FIG. 10A shows experimental design for validating immunogenicity of the string vaccine compositions in an animal model.

FIG. 10B shows a representative experimental set up of an animal model study to determine the immunogenicity of the four CorVac 2.0 strings when administered in vivo. 5 Groups (16 animals/8-10 take down at d14/8-10 at d28).

FIG. 11A is a schematic of different dosing schedules for spike vaccine (BNT162b2) and CorVac 2.0.

FIG. 11B depicts a schematic of an animal study for determining the immune responses elicited by different formulation ratios and doses of CorVac 2.0 strings with BNT162b2 in HLA-A02 transgenic mice.

FIG. 11C depicts a schematic of an animal study for determining the immune responses elicited by different formulation ratios and doses of CorVac 2.0 strings with BNT16262 in transgenic mice expressing human ACE2.

FIG. 12A demonstrates sequence variants and mutants across the spike protein in various SARS CoV-2 isolates, and the respective mapping of the vaccine epitope sequences.

FIG. 12B is a chart showing spike variant frequencies over time.

FIG. 13A demonstrates sequence variants and mutants across the nucleocapsid protein in various SARS CoV-2 isolates, and the respective mapping of the vaccine epitope sequences.

FIG. 13B is a chart showing nucleocapsid variant frequencies over time.

FIG. 14 demonstrates sequence variants and mutants across the membrane protein in various SARS CoV-2 isolates, and the respective mapping of the vaccine epitope sequences.

FIG. 15 demonstrates sequence variants and mutants across the NSP1 protein in various SARS CoV-2 isolates, and the respective mapping of the vaccine epitope sequences.

FIG. 16 demonstrates sequence variants and mutants across the NSP2 protein in various SARS CoV-2 isolates, and the respective mapping of the vaccine epitope sequences.

FIG. 17 demonstrates sequence variants and mutants across the NSP3 protein in various SARS CoV-2 isolates, and the respective mapping of the vaccine epitope sequences.

FIG. 18 demonstrates sequence variants and mutants across the NSP4 protein in various SARS CoV-2 isolates, and the respective mapping of the vaccine epitope sequences.

FIG. 19A shows CorVac 2.0-String Design for maximal CD8 and CD4 T cell responses.

FIG. 19B shows RS-C7 is enriched for known ORF1ab T cell epitopes and avoids most variants of concern/variants of interest (VOC/VOI) mutations.

FIG. 19C shows RS-C7 is enriched for known nucleocapsid and membrane T cell epitopes and avoids most variants of concern/variants of interest (VOC/VOI) mutations.

FIG. 20 shows an exemplary experimental set up outlined to test the polynucleotide strings for peptide presentation in complex with the MHC protein.

FIG. 21 depicts representative data showing an exemplary target epitope presentation verified by mass spectrometry; endogenous, in an experimental set up when the epitope is expressed on A375 cells expressing endogenous HLAs; synthetic, in an experimental set up when the epitope is expressed in cells expressing exogenous HLA.

FIG. 22 shows a diagrammatic representation of a list of epitopes identified by the above methods using mass spectrometry. The identified epitopes span the viral genome, covering epitopes of the nucleocapsid protein, ORF1ab domains and the membrane protein.

FIG. 23 shows a representation of a map of all identified CorVac 2.0 epitopes across the viral nucleocapsid, ORF1ab and membrane regions.

FIG. 24 shows representative data demonstrating Corvac 2.0 strings elicit T cell responses from the nucleocapsid region after one injection at day 0 in BALB/C mice. Immunoreactive T cells are determined by elispot assay (#spots/1×10\hat{ }6 cells). Statistical significance determined by two-way ANOVA with Sidak's multiple comparisons tests.

FIG. 25 shows representative data demonstrating Corvac 2.0 strings elicit T cell responses from the membrane region. after one injection at day 0 in BALB/C mice. Immunoreactive T cells are determined by elispot assay (#spots/1×10\hat{ }6 cells). Statistical significance determined by two-way ANOVA with Sidak's multiple comparisons tests.

FIG. 26 shows a graphical representation of the summary of performances of the different strings tested thus far. Number of stars are proportional to the statistical significance of immunogenicity of each string compared to the vehicle control.

FIG. 27 shows representative data demonstrating Corvac 2.0 strings elicit T cell responses to epitopes from the nucleocapsid region after one injection at day 0 in HLA-A2tg mice (mice that express humanized HLA-A02:01). T cell immunoreactivity was determined by ELISpot assay (#spots/1×10\hat{ }6 cells). Statistical significance was determined by two-way ANOVA with Sidak's multiple comparisons tests. Data is from mice at day 28 after injection of the string composition.

FIG. 28 shows representative data demonstrating Corvac 2.0 strings elicit T cell responses to epitopes from the membrane region after one injection at day 0 in HLA-A2tg mice (mice that express humanized HLA-A02:01). T cell immunoreactivity was determined by ELISpot assay (#spots/1×10\hat{ }6 cells). Statistical significance was determined by two-way ANOVA with Sidak's multiple comparisons tests. Data from mice at day 28 after injection of the string composition.

FIG. 29 shows representative data demonstrating Corvac 2.0 strings elicit T cell responses to epitopes from the ORF1ab region after one injection at day 0 in HLA-A2tg mice (mice that express humanized HLA-A02:01). T cell immunoreactivity was determined by ELISpot assay (#spots/1×10\hat{ }6 cells). Statistical significance was determined by two-way ANOVA with Sidak's multiple comparisons tests. Data is from mice at day 28 after injection of the string composition.

FIG. 30 is a graphical representation summarizing the statistical significance of immunogenicity of the different strings tested in the HLA-A02 transgenic mouse model compared to vehicle controls and results depicted in FIG. 27, FIG. 28 and FIG. 29. The representation indicates RS-C7 has the most T cell responses across pools in the HLA-A02 transgenic mouse model.

FIG. 31 shows a graphical representation of the RS-C7 string design showing regions of the string that elicited immune responses as determined by ELISpot assay and epitopes that were confirmed process and presented by HLA using mass spectrometry.

FIG. 32A shows a graphical representation of the string designs where the encoded nucleocapsid sequence contain, inter alia, tiled overlapping 15-mer epitope sequences with 11 amino acid overlaps.

FIG. 32B shows a graphical representation of the string designs where the encoded membrane sequence contain, inter alia, tiled 15-mer sequences with 11 amino acid overlaps.

FIG. 32C shows a graphical representation of the string designs where the encoded ORF 1ab sequence contain, inter alia, tiled 15-mer sequences with 11 amino acid overlaps.

FIG. 32D shows results from the experimental design shown in FIG. 10B indicating CorVac 2.0 strings do not raise T cell responses from the ORF1ab region in the BALB/C mouse model. In contrast, results shown in FIG. 29, (left, pool 11), (right, pool 12) indicates CorVac 2.0 strings do raise T cell responses from the ORF1ab region in an HLA-A02 transgenic mouse model. Statistical significance determined by one-way ANOVA with Sidak's multiple comparisons tests.

FIG. 32E is a graphical representation of the summary of performances of the different strings tested in the BALB/C mouse model and demonstrates that RS-C7 has the most T cell responses across pools in the BALB/C mouse model. The RS-C7 string raised the most T cell responses across pools in the BALB/C mouse model.

FIG. 33 is a graphical representation showing that the CorVac 2.0 String C7 epitopes overlap with few or no regions with mutations of variants of concern.

FIG. 34A depicts data related to assessment of B cell responses by ELISA for S1 binding antibodies in mouse serum at the indicated timepoints and serum dilutions using the indicated dosing schedules according to FIG. 11A.

FIG. 34B depicts data related to assessment of B cell responses by ELISA for NP binding antibodies in mouse serum at the indicated timepoints and serum dilutions using the indicated dosing schedules according to FIG. 11A.

FIG. 35 shows results from ELISA for serum IgG concentrations at day 14, 21 and 35 after treatment (injection) with saline control (NaCl), BNT162b2, the C7 string, or the combinations as indicated using the dosing schedules according to FIG. 11A. Results indicate that inclusion of CorVac 2.0 in a co-formulation setting does not impact Spike-specific Ab development.

FIG. 36 shows results from a pseudovirus neutralization test (pVNT) using viral particles pseudotyped with VSV envelope containing SARS-CoV-2 spike protein at day 14, 21 and 35 after treatment (injection) with saline control (NaCl), BNT162b2, the C7 string, or the combinations as indicated using the dosing schedules according to FIG. 11A. Results indicate that CorVac 2.0 does not negatively impact formation of neutralizing antibodies against the SARS-CoV-2 viral protein (Wuhan strain).

FIG. 37A shows lymph node phenotyping results at day 35 using the dosing schedules according to FIG. 11A. B cell populations are indicated by the % of CD19+ cells of the CD45+ cell population. Percentage of activated B cells was determined by the % of IgD-CD3-CD4-CD8-cells; switched B cell percentage is determined by the % of Activated/B220+IgM-CD19+CD138-cells; and % GC B cells was determined by the % of Activated/B220+IgM-CD19+CD138-cells of the total CD45+ cells in the population. Results indicate that inclusion of CorVac 2.0 in a 1:1 coformulation does not affect BNT162b2-driven B cell response, and slightly enhances it.

FIG. 37B shows lymph node phenotyping results at day 35 using the dosing schedules according to FIG. 11A. Results indicate that inclusion of CorVac 2.0 maintains the frequency of Tfh cells in the lymph node.

FIG. 38 depicts activation of splenocyte cells that have been stimulated with peptides present in BNT162b2 on day 35 and assessed by flow cytometry for the presence of the activation marker CD69 on bulk T cell population (left panel) or CD4 T cells (middle panel) or CD8 T cells (right panel) using the dosing schedules according to FIG. 11A. Results indicate that activation of T cells when restimulated with spike peptides (spike 1 pool) are increased in vaccine groups that have BNT162b2 (Group 2) and not inhibited in groups that have both CorVac 2.0 and BNT162b2 (Groups 4-7).

FIG. 39 depicts activation of splenocyte cells that have been stimulated with peptides present in BNT162b2 on day 35 and assessed by flow cytometry for the presence of the activation marker IFN-gamma on bulk T cell population (left panel) or CD8 T cells (middle panel) or CD4 T cells (right panel) using the dosing schedules according to FIG. 11A. Results indicate that activation of T cells when restimulated with spike peptides (spike 1 pool) are increased in vaccine groups that have BNT162b2 (Group 2) and not inhibited in groups that have both CorVac 2.0 and BNT162b2 (Groups 4-7) in the CD3 T cell population and CD8 T cell population, but not significantly increased in any groups in the CD4 T cell population.

FIG. 40 depicts activation of splenocyte cells that have been stimulated with peptides present in BNT162b2 on day 35 and assessed by flow cytometry for the presence of the activation marker CD69 on bulk T cell population (left panel) or CD4 T cells (middle panel) or CD8 T cells (right panel) using the dosing schedules according to FIG. 11A. Results indicate that activation of T cells when restimulated with spike peptides (spike 2 pool) are increased most dramatically in groups that have been given the BNT162b2 co-formulated in the same LNP (Groups 5-7).

FIG. 41A depicts activation of splenocyte cells that have been stimulated with peptides present in the nucleocapsid sequence present in the CorVac 2.0 string on day 35 and assessed by flow cytometry for the presence of the activation marker CD69 on bulk T cell population (left panel) or CD4 T cells (middle panel) or CD8 T cells (right panel) using the dosing schedules according to FIG. 11A. Results indicate that activation of T cells when restimulated with nucleocapsid peptides are increased in vaccine groups that have CorVac 2.0 (Group 3) and separately formulated and co-formulated strings of both BNT162b2 and CorVac 2.0 (Group 4 and 5) in the CD3 T cell population and CD8 T cell population, and modestly in the CD4 T cell population.

FIG. 41B depicts activation of splenocyte cells that have been stimulated with peptides present in the nucleocapsid sequence present in the CorVac 2.0 string on day 35 and assessed by flow cytometry for the presence of the functional marker IFN-gamma on bulk T cell population (left panel) or CD8 T cells (middle panel) or CD4 T cells (right panel) using the dosing schedules according to FIG. 11A. Results indicate that production of INF-gamma in T cells when restimulated with nucleocapsid peptides are increased in vaccine groups that have CorVac 2.0 (Group 3) and co-formulated strings of both BNT162b2 and CorVac 2.0 at the same ratio (Group 5) in the CD3 T cell population and CD4 T cell population, and modestly in the CD8 T cell population.

FIG. 42 depicts results showing Spike (N and C-terminal) antigen-specific T cell responses by ELISpot assay on samples from day 35 using the dosing schedules according to FIG. 11A. Results indicate that Ag-specific Spike T-cell responses are not inhibited by addition of CorVac 2.0 and are slightly enhanced by the 1:1 co-formulation.

FIG. 43 depicts results showing nucleocapsid, membrane and Orflab antigen-specific T cell responses by ELISpot assay on samples from day 35 using the dosing schedules according to FIG. 11A. Results indicate that CorVac 2.0 string responses are stronger in the CorVac 2.0 only group but are still present when CorVac 2.0 string is combined with BNT162b2.

FIG. 44 depicts the effect of BNT162b2 and CorVac 2.0 alone or in combination on the anti-Spike antigen T cell responses by ELISPOT at day 14 and day 35 using the dosing schedules according to FIG. 11A. The kinetic study depicted herein indicates that Spike T cell responses increase after boost in all groups.

FIG. 45 depicts the effect of booster doses on the CorVac 2.0 immune responses at day 14 and day 35 using the dosing schedules according to FIG. 11A. The kinetic study depicted herein indicates that CorVac 2.0 responses did not increase after boost.

FIG. 46 depicts polyfunctionality of CD4 and CD8 responses elicited by BNT162b2 and CorVac 2.0 using the dosing schedules according to FIG. 11A after restimulation with cognate peptide groups (Spike N-term, Spike C-term, Nucleocapsid and Membrane, as noted) assayed by flow cytometry. CD4 and CD8 T cell responses were determined to be polyfunctional by the expression of IFNγ+IL-2+TNFα+.

FIG. 47 shows a graphical representation that summarizes the effects of the different formulations indicated on the left-hand column on spike N-terminal antigen specific responses by CD4/CD8 cells using the dosing schedules according to FIG. 11A. The number of stars is proportional to the statistical significance and intensity of the response compared to the vehicle control.

FIG. 48 shows a graphical representation summarizes the effects of the different formulations indicated on the left-hand column on spike C-terminal antigen specific responses by CD4/CD8 cells using the dosing schedules according to FIG. 11A. The number of stars is proportional to the statistical significance and intensity of the response compared to the vehicle control.

FIG. 49 shows a graphical representation that summarizes the effects of the different formulations indicated on the left-hand column on nucleocapsid antigen specific responses by CD4/CD8 cells using the dosing schedules according to FIG. 11A. The number of start is proportional to the statistical significance and intensity of the response compared to the vehicle control.

FIG. 50 shows a graphical representation that summarizes the effects of the different formulations indicated on the left-hand column on membrane antigen specific responses by CD4/CD8 cells using the dosing schedules according to FIG. 11A. The number of stars is proportional to the statistical significance and intensity of the response compared to the vehicle control.

FIG. 51 depicts a schematic of an animal study for determining the immune responses elicited by different formulation ratios and doses of CorVac 2.0 strings with BNT162b2 in transgenic mice expressing human ACE2.

FIG. 52 shows data from the animal study depicted in FIG. 51, demonstrating that boosting with separately formulated CorVac2.0 at 0.3 μg dose enhances anti-spike IgG production (quantified at day 56). CorVac2.0 alone slightly boosts anti-spike IgG production, separately formulated 3:1 boosts anti-spike IgG production and inclusion of multiple doses of separately formulated CorVac2.0 does not negatively impact anti-spike IgG responses.

FIG. 53 shows data from the animal study depicted in FIG. 51, demonstrating that boosting with separately formulated CorVac2.0 at 0.3 μg dose enhances anti-spike IgG production (as measured by optical density (OD) at day 56). CorVac2.0 alone slightly boosts anti-spike IgG production, separately formulated 3:1 boosts anti-spike IgG production and inclusion of multiple doses of separately formulated CorVac2.0 does not negatively impact anti-spike IgG responses.

FIG. 54 shows data from the animal study depicted in FIG. 51, demonstrating anti-spike IgG kinetics over time. A third boost increased anti-spike IgG under all CorVac2.0 variations.

FIG. 55 shows ELISA data from the animal study depicted in FIG. 51, demonstrating anti-spike IgG levels in serum on the indicated days using the indicated serum dilutions.

FIG. 56 shows pseudovirus neutralization test (pVNT) data from the animal study depicted in FIG. 51, demonstrating that inclusion of CorVac2.0 does not negatively impact neutralizing titers on the indicated days following treatment with indicated dosing regimens. The pVNT was performed using viral particles pseudotyped with VSV envelope containing SARS-CoV-2 spike protein at the indicated dilutions.

FIG. 57 shows tetramer staining data using PBLs from the animal study depicted in FIG. 51, showing the percentage of CD8+ T cells specific to the indicated membrane and spike epitopes or epitope combinations indicated, demonstrating that anti-spike responses are not impacted by addition of CorVac2.0.

FIG. 58 shows tetramer staining data using PBLs from the animal study depicted in FIG. 51, showing the percentage of CD8+ T cells specific to the indicated spike epitopes, demonstrating that anti-spike responses are not impacted by addition of CorVac2.0.

FIG. 59 shows a spot forming assay from the animal study depicted in FIG. 51, showing the number of spots formed per 1×10\hat{ }6 cells after treatment with the indicated regimens at day 56, demonstrating that addition of CorVac2.0 in the third booster shot as a separate formulation slightly improves anti-spike T cell responses. CorVac2.0 alone slightly boosts anti-spike T cell responses, separately formulated 3:1 and 9:1 slightly boost anti-spike T cells, and inclusion of multiple doses of separately formulated CorVac2.0 does not negatively impact anti-spike T cell responses.

FIG. 60 shows a spot forming assay from the animal study depicted in FIG. 51, showing the number of spots formed per 1×10\hat{ }6 cells after treatment with the indicated regimens at day 56, demonstrating that weak anti-nucleocapsid responses were seen with 1 booster dose, and multiple doses of separately formulated BNT162b2+CorVac2.0 leads to increases in responses. Weak responses to nucleocapsid in K18-hACE2 mice with one CorVac2.0 boost was observed. Increased anti-nucleocapsid responses with increasing number of CorVac2.0 doses was also observed.

FIG. 61 shows a spot forming assay from the animal study depicted in FIG. 51, showing the number of spots formed per 1×10\hat{ }6 cells after treatment with the indicated regimens at day 56, demonstrating that the strongest anti-membrane responses were seen in separately formulated groups as a booster; multiple doses of separately formulated BNT162b2+CorVac2.0 lead to increases in responses. Strongest anti-membrane responses were seen in separately formulated conditions. Increased anti-mmbrane responses with increasing number of CorVac2.0 doses was observed.

FIG. 62 shows data from the animal study depicted in FIG. 51, in which harvested inguinal lymph nodes (a draining lympgh node (dLN)) were dissected and live cell counts were determined after treatment with the indicated regimens. Three samples with 0% viability were removed (G5, G7, G9). The data demonstrate that groups containing a CorVac2.0 boost in combination with BNT162b2 show increased live LN cell counts.

FIG. 63 shows data from the animal study depicted in FIG. 51, in which harvested lymph nodes were dissected and cell counts of the indicated cell populations as a percentage of CD45.2 cells were determined after treatment with the indicated regimens. The data demonstrate a trend for increased germinal center (GC) cells and CD27+ memory B cells in groups treated with with separately formulated CorVac2.0+BNT162.

FIG. 64A shows data from the animal study depicted in FIG. 51, in which harvested lymph nodes were dissected and cell counts of the indicated cell populations as a percentage of CD45.2 cells were determined after treatment with the indicated regimens. The data demonstrate slightly higher class-switched B cells in groups treated with with separately formulated CorVac2.0+BNT162.

FIG. 64B shows data from the animal study depicted in FIG. 51, in which harvested lymph nodes were dissected and toal cell counts of the indicated cell populations were determined after treatment with the indicated regimens. The data demonstrate slightly higher class-switched B cells in groups treated with with separately formulated CorVac2.0+BNT162

FIG. 65 shows tetramer-specific staing data from the animal study depicted in FIG. 51, from harvested lymph node samples showing the percentage of CD8+ T cells specific to spike or membrane epitopes. Also depicted is a graph showing the percentage of central memory T cells, effector memory T cells, naive T cells and effector T cells cells as a percentage of spike positive CD8 T cells. Slightly less differentiated cells in CorVac2.0 treated groups were observed.

FIG. 66 depicts a schematic of an animal study for determining the immune responses elicited by different formulation ratios and doses of CorVac 2.0 strings with BNT162b2 in HLA-A02 transgenic mice.

FIG. 67A shows a spot forming assay from the animal study depicted in FIG. 66, showing the number of spots formed per 1×10\hat{ }6 cells after treatment with the indicated regimens at day 14, demonstrating that CorVac2.0 induces the strongest immune responses at the highest dose.

FIG. 67B shows a spot forming assay from the animal study depicted in FIG. 66, showing the number of spots formed per 1×10\hat{ }6 cells after treatment with the indicated regimens at day 35, demonstrating that Spike T cell responses are enhanced with CorVac2.0 inclusion. The dose of 1 μg yielded the strongest responses.

FIG. 67C shows a spot forming assay from the animal study depicted in FIG. 66, showing the number of spots formed per 1×10\hat{ }6 cells over time. Improved T cell responses to all regions were observed after the boost.

FIG. 67D shows a co-culture spot forming assay from the animal study depicted in FIG. 66, showing the number of spots formed per 1×10\hat{ }6 cells after treatment with the indicated regimens at day 35. Spike responses were seen in CD4 and CD8 T cells. Spike CD8 T cells show more cytokine secretion and degranulation than CD4 T cells.

FIG. 67E shows a co-culture spot forming assay from the animal study depicted in FIG. 66, showing the number of spots formed per 1×10\hat{ }6 cells after treatment with the indicated regimens at day 35. Membrane responses were seen in CD4 and CD8 T cells and were strongest at the highest dose. IL2 secretion was seen in CD4 and CD8 T cells.

FIG. 67F shows a spot forming assay from the animal study depicted in FIG. 66, showing the number of spots formed per 1×10\hat{ }6 cells after treatment with the indicated regimens at day 56. Spike responses were not negatively impacted by CorVac2.0. CorVac2.0 alone boosted spike responses. Boost with CorVac2.0 alone increased spike responses. No statistically significant changes in spike responses were seen with inclusion of CorVac2.0. The strongest membrane responses were observed in the highest dose groups. The strongest N responseswere observed in CorVac2.0 alone at the highest dose. The strongest Orf1ab responses were observed in CorVac2.0 alone and the separately formulated 3:1 dose.

FIG. 67G shows a co-culture spot forming assay from the animal study depicted in FIG. 66, showing the number of spots formed per 1×10\hat{ }6 cells after treatment with the indicated regimens at day 56. Spike responses were seen in CD4 and CD8 T cells and some were boosted by inclusion of CorVac2.0.

FIG. 67H shows a co-culture spot forming assay from the animal study depicted in FIG. 66, showing the number of spots formed per 1×10\hat{ }6 cells after treatment with the indicated regimens at day 56. Polyfunctional spike CD4 and CD8 T cells were slightly enhanced in higher dose CorVac2.0 conditions.

FIG. 67I shows a co-culture spot forming assay from the animal study depicted in FIG. 66, showing the number of spots formed per 1×10\hat{ }6 cells after treatment with the indicated regimens at day 56. Membrane responses seen more strongly in CD8 T cells.

FIG. 67J shows a co-culture spot forming assay from the animal study depicted in FIG. 66, showing the number of spots formed per 1×10\hat{ }6 cells after treatment with the indicated regimens at day 56. Polyfunctional membrane CD4 and CD8 T cells were observed.

FIG. 68A shows phenotyping data from the animal study depicted in FIG. 66, in which harvested lymph nodes were dissected and cell counts of the indicated cell populations as a percentage of CD45.2 cells were determined after treatment with the indicated regimens at day 14. Memory B cells: CD3-IgD-IgM-CD27+; Activated B cells: IgD-CD3-CD4-CD8-; Switched B cells: IgD-CD3-CD4-CD8-B220+IgM-CD19+CD138-. GC B cells: IgD-CD3-CD4-CD8-B220+IgM-CD19+CD138-CD95+CD38-.

FIG. 68B shows phenotyping data from the animal study depicted in FIG. 66, in which harvested lymph nodes were dissected and cell counts of the indicated cell populations as a percentage of CD45.2 cells were determined after treatment with the indicated regimens at day 35. Memory B cells: CD3-IgD-IgM-CD27+; Activated B cells: IgD-CD3-CD4-CD8-; Switched B cells: IgD-CD3-CD4-CD8-B220+IgM-CD19+CD138-. GC B cells: IgD-CD3-CD4-CD8-B220+IgM-CD19+CD138-CD95+CD38-. The addition of CorVac2.0 to BNT162b2 slightly enhances class-switching and GC B cells. CorVac2.0 leads to a slight increase in Tfh cells.

FIG. 68C shows phenotyping data from the animal study depicted in FIG. 66, in which harvested lymph nodes were dissected and cell counts of the indicated cell populations as a percentage of CD45.2 cells were determined after treatment with the indicated regimens at day 56. Memory B cells: CD3-IgD-IgM-CD27+; Activated B cells: IgD-CD3-CD4-CD8-; Switched B cells: IgD-CD3-CD4-CD8-B220+IgM-CD19+CD138-. GC B cells: IgD-CD3-CD4-CD8-B220+IgM-CD19+CD138-CD95+CD38-. Boosting with CorVac2.0 alone enhances B cell responses compared to no boost. CorVac2.0 does not significantly affect B cells responses in combination with BNT162b2.

FIG. 69 shows an exemplary clinical study design to evaluate safety of CorVac 2.0 vaccines in healthy human subjects.

FIG. 70 shows an exemplary clinical study design to evaluate safety of CorVac 2.0 vaccines in immunocompromised human subjects.

FIG. 71 shows an exemplary clinical study design to evaluate safety of CorVac 2.0 vaccines in immunocompromised human subjects.

FIG. 72 demonstrates sequence variants and mutants across the Spike protein in various SARS CoV-2 isolates as indicated on the right hand side. The data shows that the Spike protein is highly mutated putitively due to selective pressure.

FIG. 73 demonstrates sequence variants and mutants across the nucleocapsid (N) and membrane (M) protein in various SARS CoV-2 isolates as indicated on the right hand side, and the respective mapping of the vaccine epitope sequences in encoded by CorVac 2.0. The data shows that the CorVac2.0 vaccine sequence is rarely impacted by variant mutations in N, M.

FIG. 74 demonstrates sequence variants and mutants across the ORF 1ab in various SARS COV-2 isolates as indicated on the right hand side, and the respective mapping of the vaccine epitope sequences in encoded by CorVac 2.0. The data shows that the CorVac2.0 vaccine sequence is rarely impacted by variant mutations in ORF1ab.

FIG. 75A shows a representation of a map of an exemplary CorVac2.0 RS-C7 string depicting linkers, the SEC domain, the transmembrane (TM) domain and the viral epitopes contained within the string, including nucleocapsid epitopes, ORF1ab epitopes, and membrane epitopes.

FIG. 75B shows a representation of a map of an exemplary CorVac2.0 RS-C7 string depicting the viral epitopes contained within the string that were observed as being presented by mass spectrometry (MS), including nucleocapsid epitopes, ORF1ab epitopes, and membrane epitopes.

FIG. 76 shows a representation of an MS-based HLA-I cleavage predictor used to optimize ordering of candidate ORF1ab sequences, adding as few linkers as possible while retaining efficient epitope cleavage (top) and 18 ORF1ab epitopes optimized for GSS linker contexts.

FIG. 77A-C shows a representation of a map of an exemplary CorVac2.0 RS-C7 string Antigens chosen for CorVac 2.0 are rarely mutated: across all WHO-designated variants, RS-C7 is only impacted by 3 mutations, leaving the vast majority of epitopes unchanged. As shown in FIG. 77B-C, even in the highly mutated Omicron variants, only one mutation impacts CorVac 2.0 strings (in contrast to ~39 amino acid changes in Spike protein).

FIG. 78A-78B shows kinetics of the antibody concentration against the Spike protein.

FIG. 79 depicts an exemplary structure of RS C7, and illustrates certain factors that were considered when designing a CorVac 2.0 string. “Count”, as used in FIG. 79, refers to the number of pMHC-allele pairs that overlap with an indicated residue of a protein sequence. Counts can be determined by reference to a database of epitopes (e.g., a database of epitopes that have been observed, for example, in experimental studies, and/or predicted). “Entropy” as used in FIG. 79 refers to Shannon entropy, and is a measure of conservation level (where lower entropy indicates higher conservation).

FIG. 80A depicts epitopes present in an exemplary CorVac 2.0 string (e.g., RS C7 string) that were observed via mass spectrometery as processed and presented by MHC complexes.

FIG. 80B depicts data for one exemplary epitope observed by mass spectrometry (epitope 23 of Table 19).

FIG. 81 depicts a study design to test the immunogenicity of various BNT162b2+CorVac2.0 string dosing regimens in K18-hACE2 mice.

FIG. 82A-82B show anti-Spike protein, IgG concentrations measured over time, from serum samples collected from the experiment depicted in FIG. 81. IgG concentrations measured by ELISA. Error bars indicate standard error of the mean.

FIG. 83A-83B show neutralization titers measured from the serum samples collected from the experiment depicted in FIG. 81. Neutralization titers were measured using a pseudovirus neutralization assay, using pseudoviruses comprising an SARS-CoV-2 S protein from a Wuhan stain.

FIG. 84 depicts an experimental protocol for measuring the efficacy of a CorVac2.0 string, administered alone or in combination with RNA encoding a SARS-CoV-2 S protein in Syrian Hamsters.

FIG. 85 shows results from the study depicted in FIG. 84. Specifically, FIG. 85 shows changes in body weight of hamsters, following challenge with SARS-CoV-2 (Wuhan strain).

FIG. 86 shows an exemplary protocol for clonotype analysis of Spike-specific B cells and T cells.

FIG. 87 summarizes T cell clonotypes observed in animals administered (i) two doses of RNA encoding a SARS-CoV-2 S protein (BNT162b2) or (ii) two doses of RNA encoding a SARS-CoV-2 S protein and one dose of CorVac2.0 (String C7). Circles correspond to samples having the same clonotype, with clonotypes in the same “clonotype cluster” (i.e., clonotypes having similar sequences) indicated with lines connecting dots. As shown in the figure, administration of a CorVac2.0 construct increases the trend towards higher clonal expansion, but does not negatively impact clonality.

FIG. 88 shows an exemplary clinical study design to evaluate safety of CorVac 2.0 vaccines in healthy human subjects. “Spike Vac” refers to RNA encoding a SARS-CoV-2 S protein and comprising one or more mutations that stabilize the prefusion conformation (e.g., one or more mutations disclosed herein or known in the art); in the exemplary clinical study design, a bivalent vaccine is administered, comprising RNA encoding a SARS-CoV-2 S polypeptide of a Wuhan strain and a SARS-CoV-2 S polypeptide comprising one or more mutations characteristic of a BA.4/5 Omicron variant (e.g., one or more mutations disclosed herein). ^{a}Further dosing of subjects or opening of new cohorts may proceed if acceptable tolerability is shown and no stopping/pausing rules are met. ^{b}An exemplary Spike Vac Bivalent is BNT162b2 Bivalent (Wuhan+OMI BA.4/BA.5). Abbreviations: d=days; h=hour(s); FIH=first-in-human; IRC=Internal Review Committee; N=number of subjects; OMI=Omicron; WT=Wild Type. Trial population: Healthy volunteers aged 18-55 years, who have been previously vaccinated with at least three doses of any authorized COVID19 RNA vaccine (the last COVID19 RNA vaccine dose must have been administered at least 4 months before Visit 1). Subjects who have had SARS-CoV-2 infection 60 d or more prior to randomization are not excluded from the trial.

### DETAILED DESCRIPTION

Described herein are novel therapeutics and vaccines based on viral epitopes. Accordingly, the present disclosure described herein provides peptides, polynucleotides encoding the peptides, and peptide binding agents that can be used, for example, to stimulate an immune response to a viral antigen, to create an immunogenic composition or vaccine for use in treating or preventing a viral infection.

### Definitions

To facilitate an understanding of the present disclosure, a number of terms and phrases are defined below.

“Viral antigens” refer to antigens encoded by a virus. They include, but are not limited to, antigens of coronaviruses, such as COVID19.

Throughout this disclosure, “binding data” results can be expressed in terms of “IC50.” IC50 is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC50, relative to the IC50 of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

The term “derived” when used to discuss an epitope is a synonym for “prepared.” A derived epitope can be from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

A “diluent” includes sterile liquids, such as water and oils, including those of petroleum, animal, vegetable or synthetic origin, such as peanut oil, soybean oil, mineral oil, sesame oil and the like. Water is also a diluent for pharmaceutical compositions. Saline solutions and aqueous dextrose and glycerol solutions can also be employed as diluents, for example, in injectable solutions.

An “epitope” is the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes.

It is to be appreciated that proteins or peptides that comprise an epitope or an analog described herein as well as additional amino acid(s) are still within the bounds of the present disclosure. In certain embodiments, the peptide comprises a fragment of an antigen.

In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 amino acid residues.

“Human Leukocyte Antigen” or “HLA” is a human class I or class II Major Histocompatibility Complex (MITC) protein (see, e.g., Stites, et al., IMMUNOLOGY, 8TH ED., Lange Publishing, Los Altos, Calif. (1994).

An “HLA supertype or HLA family”, as used herein, describes sets of HLA molecules grouped on the basis of shared peptide-binding specificities. HLA class I molecules that share somewhat similar binding affinity for peptides bearing certain amino acid motifs are grouped into such HLA supertypes. The terms HLA superfamily, HLA supertype family, HLA family, and HLA xx-like molecules (where “xx” denotes a particular HLA type), are synonyms.

The terms “identical” or percent “identity,” in the context of two or more peptide sequences or antigen fragments, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL), helper T lymphocyte (HTL) and/or B lymphocyte response. Thus, immunogenic peptides described herein are capable of binding to an appropriate HLA molecule and thereafter inducing a CTL (cytotoxic) response, or a HTL (and humoral) response, to the peptide.

As used herein, a “chimeric antigen receptor” or “CAR” refers to an antigen binding protein in that includes an immunoglobulin antigen binding domain (e.g., an immunoglobulin variable domain) and a T cell receptor (TCR) constant domain. As used herein, a “constant domain” of a TCR polypeptide includes a membrane-proximal TCR constant domain, and may also include a TCR transmembrane domain and/or a TCR cytoplasmic tail. For example, in some embodiments, the CAR is a dimer that includes a first polypeptide comprising a immunoglobulin heavy chain variable domain linked to a TCR-beta constant domain and a second polypeptide comprising an immunoglobulin light chain variable domain (e.g., a lc or 2\., variable domain) linked to a TCRα constant domain. In some embodiments, the CAR is a dimer that includes a first polypeptide comprising a immunoglobulin heavy chain variable domain linked to a TCRα constant domain and a second polypeptide comprising an immunoglobulin light chain variable domain linked to a TCRβ constant domain.

The phrases “isolated” or “biologically pure” refer to material which is substantially or essentially free from components which normally accompany the material as it is found in its native state. Thus, peptides described herein do not contain some or all of the materials normally associated with the peptides in their in situ environment. An “isolated” epitope refers to an epitope that does not include the whole sequence of the antigen from which the epitope was derived. Typically, the “isolated” epitope does not have attached thereto additional amino acid residues that result in a sequence that has 100% identity over the entire length of a native sequence. The native sequence can be a sequence such as a viral antigen from which the epitope is derived. Thus, the term “isolated” means that the material is removed from its original environment (e.g., the natural environment if it is naturally occurring). For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. RNA molecules include in vivo or in vitro RNA transcripts of the DNA molecules described herein, and further include such molecules produced synthetically.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, FUNDAMENTAL IMMUNOLOGY, 3.sup.RD ED., Raven Press, New York (1993).

A “native” or a “wild type” sequence refers to a sequence found in nature. Such a sequence can comprise a longer sequence in nature.

A “T cell epitope” is to be understood as meaning a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

A “receptor” is to be understood as meaning a biological molecule or a molecule grouping capable of binding a ligand. A receptor may serve, to transmit information in a cell, a cell formation or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex”, including a peptide- or peptide fragment-presenting MHC molecule of class I or of class II.

“Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

The terms “peptide” and “peptide epitope” are used interchangeably with “oligopeptide” in the present specification to designate a series of residues connected one to the other, typically by peptide bonds between the α-amino and carboxyl groups of adjacent amino acid residues.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

A “PanDR binding” peptide, a “PanDR binding epitope” is a member of a family of molecules that binds more than one HLA class II DR molecule.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable. The term “motif refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) fora class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

A “supermotif’ is a peptide binding specificity shared by HLA molecules encoded by two or more HLA alleles. In some embodiments, a supermotif-bearing peptide described herein is recognized with high or intermediate affinity (as defined herein) by two or more HLA antigens.

The term “naturally occurring” as used herein refers to the fact that an object can be found in nature. For example, a peptide or nucleic acid that is present in an organism (including viruses) and can be from a source in nature and which has not been intentionally modified by man in the laboratory is naturally occurring.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cell infected with a virus. A vaccine may be used for the prevention or treatment of a disease.

A “protective immune response” or “therapeutic immune response” refers to a CTL and/or an HTL response to an antigen derived from an pathogenic antigen (e.g., a viral antigen), which in some way prevents or at least partially arrests disease symptoms, side effects or progression. The immune response can also include an antibody response which has been facilitated by the stimulation of helper T cells.

“Antigen processing” or “processing” refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells.

The main types of professional antigen-presenting cells are dendritic cells, which have the broadest range of antigen presentation, and are probably the most important antigen presenting cells, macrophages, B-cells, and certain activated epithelial cells.

Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antiviral immunity.

Dendritic cells are conveniently categorized as “immature” and “mature” cells, which can be used as a simple way to discriminate between two well characterized phenotypes. However, this nomenclature should not be construed to exclude all possible intermediate stages of differentiation.

Immature dendritic cells are characterized as antigen presenting cells with a high capacity for antigen uptake and processing, which correlates with the high expression of Fey receptor and mannose receptor. The mature phenotype is typically characterized by a lower expression of these markers, but a high expression of cell surface molecules responsible for T cell activation such as class I and class II MHC, adhesion molecules (e. g. CD54 and CD11) and costimulatory molecules (e. g., CD40, CD80, CD86 and 4-1 BB).

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part.

In the formulae representing selected specific embodiments of the present disclosure, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

The terms “polynucleotide” and “nucleic acid” are used interchangeably herein and refer to polymers of nucleotides of any length, and include DNA and RNA, for example, mRNA. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered is mRNA.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides described herein are substantially identical, meaning they have at least 70%, at least 75%, at least 80%, at least 85%, at least 90%, and in some embodiments at least 95%, at least 96%, at least 97%, at least 98%, at least 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is substantially pure. In one embodiment, a “polynucleotide” encompasses a PCR or quantitative PCR reaction comprising the polynucleotide amplified in the PCR or quantitative PCR reaction.

The term “substantially pure” as used herein refers to material which is at least 50% pure (i.e., free from contaminants), at least 90% pure, at least 95% pure, at least 98% pure, or at least 99% pure.

The term ‘subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented.

As used in the present disclosure and embodiments, the singular forms “a”, “an” and “the” include plural forms unless the context clearly dictates otherwise.

The term “therapeutic” refers a composition that is used to treat or prevent a disease or a condition, such as viral infection, e.g., in some embodiments coronaviral infection. For example, in some embodiments, a therapeutic is or comprises a vaccine. In some embodiments, a therapeutic may be a drug, e.g., a small molecule drug. A therapeutic may be administered to a subject in need thereof, to prevent a disease or an infection, or to reduce or ameliorate one or more symptoms associated with a disease. A therapeutic may also be considered to treat at least a symptom of the disease.

It is understood that terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the present disclosure. Nothing herein is intended as a promise.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “2019 SARS-CoV 2” when, for example, referring to a virus, includes, but is not limited to, the 2019 SARS-CoV 2 virus and any mutant or variant thereof. A variant of a 2019 SARS-CoV 2 virus, or simply a variant as referred to here may mean a virus strain that is mutated with respect to the originally sequenced 2019 SARS-CoV 2 virus strain, e.g., the Wuhan strain. A mutation can be present in a coding region, e.g., spike protein encoding region, a nucleocapsid protein encoding region or any viral protein encoding region. A mutation may be in the genome of the virus that changes or has the potential to change its infectivity and/or virulence.

2019 SARS-CoV 2 variants include, but are not limited to, the variants in the table below:

TABLE A
Exemplary 2019 SARS-COV 2 variants

| Name | SNPs | Notes |
|---|---|---|
| Alpha (B.1.1.7; | orf1ab:T1001I | Initially a UK lineage, associated with a variant of concern with |
| 201; S.501Y.V1) | orflab:A1708D | N501Y, P681H and numerous other mutations. |
| | orf1ab:I2230T | Pangolin assigns B.1.1.7 to sequences with at least 5 of the 17 |
| | del : 11288:9 | defining B.1.1.7 SNPs |
| | del:21765:6 | |
| | del:21991:3 | |
| | S:N501Y | |
| | S:A570D | |
| | S:P681H | |
| | S:T716I | |
| | S:S982A | |
| | S:D1118H | |
| | Orf8:Q27* | |
| | Orf8:R52I | |
| | Orf8 : Y73C | |
| | N:D3L | |
| | N:S235F | |
| | S:H69- | Announced on the 14 Dec. 2020, this variant appears to have arisen |
| | S:V70- | and/or initially expanded in the South East of England. |
| | S : Y144- | |
| | S:N501Y | |
| | S:A570D | |
| | S:D614G | |
| | S:P681H | |
| | S:T716I | |
| | S:S982A | |
| | S:D1118H | |
| | ORF1a:T1001I | |
| | ORF1a:A1708D | |
| | ORF1a:I2230T | |
| | ORF1a:S3675- | |
| | ORF1a:G3676- | |
| | ORF1a:F3677- | |
| | N:D3L | |
| | N:R203K | |
| | N:G204R | |
| | N:S235F | |
| | ORF1b:P314L | |
| | ORF8:Q27* | |
| | ORF8:R52I | |
| | ORF8:Y73C | |
| Beta (B.1.351; | E:P71L | A lineage first identified in South Africa and defined by new |
| 20H; S.501Y.V2) | N:T205I | variant of concern 501Y.V2 |
| | orf1a:K1655N | Pangolin assigns B.1.351 to sequences with at least 5 of the 9 |
| | S:D80A | defining B.1.351 SNPs |
| | S:D215G | |
| | S:K417N | |
| | S:A701V | |
| | S:N501Y | |
| | S:E484K | |
| | S:D80A | Announced in December 2020, 20H (Beta, V2) originated and/or |
| | S:D215G | initially expanded in South Africa |
| | S:L241- | |
| | S:L242- | |
| | S:A243- | |
| | S:K417N | |
| | S:E484K | |
| | S:N501Y | |
| | S:D614G | |
| | S:A701V | |
| | ORF3a:Q57H | |
| | ORF1a:T265I | |
| | ORF1a:K1655N | |
| | ORF1a:K3353R | |
| | ORF1a:S3675- | |
| | ORF1a:G3676- | |
| | ORF1a:F3677- | |
| | N:T205I | |
| | ORF1b:P314L | |
| | E:P71L | |
| Delta (B.1.617.2; | S:T19R | A lineage circulating with variants of biological significance |
| 21A; | S:L452R | S:P681R and S:L452R, first detected in India and international |
| 21A/S:478K) | S:T478K | cases with travel history from India. |
| | S:P681R | Pangolin assigns B.1.617.2 to sequences with at least 5 of the 15 |
| | S:D950N | defining B.1.617.2 SNPs |
| | ORF3a:S26L | |
| | M:I82T | |
| | ORF7a:V82A | |
| | ORF7a:T120I | |
| | N:D63G | |
| | N:R203M | |
| | N:D377Y | |
| | S:T19R | B.1.617 was first detected in late 2020 in India, and has appeared to |
| | S:E156- | expand rapidly |
| | S:F157- | |
| | S:R158G | |
| | S:L452R | |
| | S:T478K | |
| | S:D614G | |
| | S:P681R | |
| | S:D950N | |
| | ORF1b:P314L | |
| | ORF1b:P1000L | |
| | M:I82T | |
| | N:D63G | |
| | N:R203M | |
| | N:D377Y | |
| | ORF3a:S26L | |
| | ORF7a : V82A | |
| | ORF7a:T120I | |
| Gamma | orflab:S1188L | A lineage first identified in Brazil with variants of biological |
| (B.1.1.28.1; P.1; | orflab:K1795Q | significance E484K, N501Y and K417T |
| 20J; S.501Y.V3) | del : 11288:9 | Pangolin assigns P.1 to sequences with at least 10 of the 17 defining |
| | S:L18F | P.1 SNPs |
| | S:T20N | |
| | S:P26S | |
| | S:D138Y | |
| | S:R190S | |
| | S:K417T | |
| | S:E484K | |
| | S:N501Y | |
| | S:H655Y | |
| | S:T1027I | |
| | orf3a:G174C | |
| | orf8:E92K | |
| | N:P80R | |
| | S:L18F | Announced in January 2021, originated and/or initially expanded in |
| | S:T20N | Brazil, and was particularly associated with Manaus, Amazonas |
| | S:P26S | |
| | S:D138Y | |
| | S:R190S | |
| | S:K417T | |
| | S:E484K | |
| | S:N501Y | |
| | S:D614G | |
| | S:H655Y | |
| | S:T1027I | |
| | S:V1176F | |
| | ORF3a:S253P | |
| | ORF1a:S1188L | |
| | ORF1a:K1795Q | |
| | ORF1a:S3675- | |
| | ORF1a:G3676- | |
| | ORF1a:F3677- | |
| | N:P80R | |
| | N:R203K | |
| | N:G204R | |
| | ORF1b:P314L | |
| | ORF1b:E1264D | |
| | ORF8:E92K | |
| Epsilon (21C; | S:S13I | Was first identified in California and appears to have increased in |
| 20C/S:452R) | S:W152C | frequency |
| | S:L452R | |
| | S:D614G | |
| | ORF1a:I4205V | |
| | ORF1b:D1183Y | |
| Eta (B.1.525; | orflab:L4715F | International lineage with variants of biological significance |
| 21D; | S:Q52R | E484K, Q677H, F888L and a similar suite of deletions to B.1.1.7. |
| 20A/S:484K) | S:E484K | |
| | S:Q677H | |
| | S:F888L | |
| | E:L21F | |
| | E:I82T | |
| | del11288:9 | |
| | del:21765:6 | |
| | del:28278:3 | |
| | S:Q52R | Appears to have arisen in late 2020, and as of March 2021 has been |
| | S:A67V | detected in North America, Europe, Asia, Africa, and Australia |
| | S:H69- | |
| | S:V70- | |
| | S:Y144- | |
| | S:E484K | |
| | S:D614G | |
| | S:Q677H | |
| | S:F888L | |
| | ORF1b:P314F | |
| | N:S2- | |
| | N:D3Y | |
| | N:A12G | |
| | N:T205I | |
| | M:182T | |
| | ORF1a:T2007I | |
| | ORF1a:S3675- | |
| | ORF1a:G3676- | |
| | ORF1a:F3677- | |
| | E:L21F | |
| | ORF6:F2- | |
| Iota (B.1.526; | S:L5F | Largely found within the USA, particularly in New York state, and |
| 21F; | S:T95I | seems to have arisen in late 2020. |
| 20C/S:484K) | S:D253G | A lineage predominantly circulating in New York but with a few |
| | S:E484K | exports to other countries. Characterised by spike mutations T95I |
| | S:D614G | and D253G, plus others. The most frequent spike mutation pattern |
| | S:A701V | is L5F T95I D253G E484K D614G A701V, with a smaller fraction |
| | ORF1b:P314L | having S477N instead of E484K. Spike mutation E484K is present |
| | ORF1b:Q1011H | in about half of this lineage (as of 2021 Feb. 10) |
| | ORF3a:P42L | |
| | ORF3a:Q57H | |
| | ORF1a:T265I | |
| | ORF1a:L3201P | |
| | ORF1a:S3675- | |
| | ORF1a:G3676- | |
| | ORF1a:F3677- | |
| | N:P199L | |
| | N:M234I | |
| | ORF8:T11I | |
| Kappa | S:E154K | Predominantly India lineage with 484Q |
| (B.1.617.1; 21B; | S:L452R | B.1.617 was first detected in late 2020 in India, and has appeared to |
| 21A/S:154K) | S:E484Q | expand rapidly |
| | S:D614G | |
| | S:P681R | |
| | S:Q1071H | |
| | ORF1b:P314L | |
| | ORF1b:G1129C | |
| | ORF1b:M1352I | |
| | ORF1b:K2310R | |
| | ORF1b:S2312A | |
| | N:R203M | |
| | N:D377Y | |
| | M:I82S | |
| | ORF3a:S26L | |
| | ORF1a:T1567I | |
| | ORF1a:T3646A | |
| | ORF7a : V82A | |
| Lambda (C.37; | S:G75V | Lineage in Peru, Chile, USA and Germany. |
| 21G; B.1.1.1.37) | S:T76I | |
| | S:R246- | |
| | S:S247- | |
| | S:Y248- | |
| | S:L249- | |
| | S:T250- | |
| | S:P251- | |
| | S:G252- | |
| | S:D253N | |
| | S:L452Q | |
| | S:F490S | |
| | S:D614G | |
| | S:T859N | |
| | ORF1a:T1246I | Appears to have arisen in late 2020 in South America. Early |
| | ORF1a:P2287S | sequences are predominantly from Peru and Chile. |
| | ORF1a:F2387V | |
| | ORF1a:L3201P | |
| | ORF1a:T3255I | |
| | ORF1a:G3278S | |
| | ORF1a:S3675- | |
| | ORF1a:G3676- | |
| | ORF1a:F3677- | |
| | ORF1b:P314L | |
| | N:P13L | |
| | N:R203K | |
| | N:G204R | |
| | N:G214C | |
| Mu (B.1.621; | S:T95I | Lineage predominantly in Colombia with several spike mutations. |
| 21H) | S:Y144S | Appears to have arisen in early 2021 in South America. |
| | S:Y145N | |
| | S:R346K | |
| | S:E484K | |
| | S:N501Y | |
| | S:D614G | |
| | S:P681H | |
| | S:D950N | |
| | ORF1a:T1055A | |
| | ORF1a:T1538I | |
| | ORF1a:T3255I | |
| | ORF1a:Q3729R | |
| | ORF1b:P314L | |
| | ORF1b:P1342S | |
| | N:T205I | |
| | ORF3a:Q57H | |
| | ORF3a:V256I | |
| | ORF3a:N257Q | |
| | ORF3a:P258* | |
| | ORF8:T11K | |
| | ORF8:P38S | |
| | ORF8:S67F | |
| Omicron | del:6513:3 | A lineage detected in a number of countries with a high number of |
| (B.1.1.529) | del:11283:9 | spike protein mutations. Since initial detection, a number of |
| | nuc:C241T | Omicron variants have arisen, including BA.1, BA.2, BA.2.12.1, |
| | ORF1A:K856R | BA.4, BA.5, and BA.2.75 (mutations shown in the present table |
| | nuc:C3037T | correspond to the BA.1 Omicron variant). |
| | nuc:T5386G | First reported to WHO from South Africa on 24 November 2021. |
| | ORF1A:A2710T | |
| | ORF1A:T3255I | |
| | ORF1A:P3395H | |
| | ORF1A:I3758V | |
| | nuc:T13195C | |
| | ORF1B:P314L | |
| | nuc:C15240T | |
| | ORF1B:I1566V | |
| | S:A67V | |
| | S:T95I | |
| | S:G339D | |
| | S:S371L | |
| | S:S373P | |
| | S:K417N | |
| | S:N440K | |
| | S:G446S | |
| | S:S477N | |
| | S:T478K | |
| | S:E484A | |
| | S:Q493R | |
| | S:G496S | |
| | S:Q498R | |
| | S:N501Y | |
| | S:T547K | |
| | S:D614G | |
| | S:H655Y | |
| | S:N679K | |
| | S:P681H | |
| | S:N764K | |
| | S:D796Y | |
| | S:N856K | |
| | S:Q954H | |
| | S:N969K | |
| | nuc:C25000T | |
| | E:T9I | |
| | M:D3G | |
| | M:Q19E | |
| | M:A63T | |
| | nuc:A27259C | |
| | nuc:C27807T | |
| | N:RG203KR | |

In some embodiments, sequencing methods may be used to identify virus specific epitopes. Any suitable sequencing method can be used according to the present disclosure, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present disclosure mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the present disclosure e.g. those described in detail in WO 2012/159643.

In certain embodiments a viral epitope peptide described herein molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein. In specific embodiments, a viral epitope peptide molecule is equal to or less than 100 amino acids.

In some embodiments, viral epitope peptides described herein for MHC Class I are 13 residues or less in length and usually consist of between about 8 and about 11 residues, particularly 9 or 10 residues. In some embodiments, viral epitope peptides described herein for MHC Class II are 9-24 residues in length.

A longer viral protein epitope peptide can be designed in several ways. In some embodiments, when HLA-binding peptides are predicted or known, a longer viral protein epitope peptide could consist of (1) individual binding peptides with extensions of 2-5 amino acids toward the N- and C-terminus of each corresponding peptide; or (2) a concatenation of some or all of the binding peptides with extended sequences for each. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long viral epitope peptide.

In some embodiments, the viral epitope peptides and polypeptides bind an HLA protein (e.g., HLA class I or HLA class II). In specific embodiments the viral epitope peptide or polypeptide has an IC50 of at least less than 5000 nM, at least less than 500 nM, at least less than 100 nM, at least less than 50 nM or less.

In some embodiments, a viral protein epitope peptide described herein can be in solution, lyophilized, or can be in crystal form.

In some embodiments, a viral protein epitope peptide described herein can be prepared synthetically, by recombinant DNA technology or chemical synthesis, or can be from natural sources such as native viruses. Epitopes can be synthesized individually or joined directly or indirectly in a peptide. Although a viral epitope peptide described herein will be substantially free of other naturally occurring host cell proteins and fragments thereof, in some embodiments the peptide can be synthetically conjugated to be joined to native fragments or particles.

In some embodiments, a viral protein epitope peptide described herein can be prepared in a wide variety of ways. In some embodiments, the peptides can be synthesized in solution or on a solid support according to conventional techniques. Various automatic synthesizers are commercially available and can be used according to known protocols. (See, for example, Stewart & Young, Solid Phase Peptide Synthesis, 2D. ED., Pierce Chemical Co., 1984). Further, individual peptides can be joined using chemical ligation to produce larger peptides that are still within the bounds of the present disclosure.

Alternatively, recombinant DNA technology can be employed wherein a nucleotide sequence which encodes a peptide inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Thus, recombinant peptides, which comprise or consist of one or more epitopes described herein, can be used to present the appropriate T cell epitope.

In one aspect, the present disclosure described herein also provides compositions comprising one, at least two, or more than two viral epitope peptides. In some embodiments a composition described herein contains at least two distinct peptides. In some embodiments, the at least two distinct peptides are derived from the same polypeptide. By distinct polypeptides is meant that the peptide vary by length, amino acid sequence or both. The peptides are derived from any polypeptide known to or have been found to contain a viral-specific epitope.

### Viral Epitope Polynucleotides

Polynucleotides encoding each of the peptides described herein are also within the scope of the present disclosure. As appreciated by one of ordinary skill in the art, various nucleic acids can encode the same peptide due to the redundancy of the genetic code. Each of these nucleic acids falls within the scope of the present disclosure. This embodiment of the present disclosure comprises DNA and RNA, for example, mRNA, and in certain embodiments a combination of DNA and RNA. In one embodiment, the mRNA is a self-amplifying mRNA. (Brito et al., Adv. Genet. 2015; 89:179-233). It is to be appreciated that any polynucleotide that encodes a peptide described herein falls within the scope of this present disclosure.

The term “RNA” includes and in some embodiments relates to “mRNA”. The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In one embodiment, the mRNA is self-amplifying mRNA. In the context of the present disclosure, mRNA may be generated by in vitro transcription from a DNA template. The in vitro transcription methodology is known to the skilled person. For example, there is a variety of in vitro transcription kits commercially available.

The stability and/or translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448, PCT/EP15/073180, and PCT/EP2015/065357, each of which are incorporated herein by reference. In order to increase expression of the RNA used according to the present disclosure, it may be modified within the coding region, i.e. the sequence encoding the expressed peptide or protein, without altering the sequence of the expressed peptide or protein, so as to increase the GC-content to increase mRNA stability and to perform a codon optimization and, thus, enhance translation in cells.

The term “modification” in the context of the RNA used in the present disclosure includes any modification of an RNA which is not naturally present in said RNA. In one embodiment of the present disclosure, the RNA used according to the present disclosure does not have uncapped 5′-triphosphates. Removal of such uncapped 5′-triphosphates can be achieved by treating RNA with a phosphatase. The RNA according to the present disclosure may have modified ribonucleotides in order to increase its stability and/or decrease cytotoxicity. For example, in one embodiment, in the RNA used according to the present disclosure cytidine may be substituted by 5-methylcytidine; 5-methylcytidine is substituted partially or completely, for example, completely, for cytidine. Alternatively, or additionally, in one embodiment, in the RNA used according to the present disclosure uridine may be substituted by a modified uridine. In some embodiments pseudouridine or 1-methyl pseudouridine; pseudouridine or 1-methyl pseudouridine is substituted partially or completely, for example, completely, for uridine.

In one embodiment the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, to the 7-methylguanosine cap (m G). In the context of the present disclosure, the term “5′-cap” includes a 5′-cap analog that resembles the RNA cap structure and is modified to possess the ability to stabilize RNA and/or enhance translation of RNA if attached thereto, in vivo and/or in a cell.

In certain embodiments, an mRNA encoding a viral epitope is administered to a subject in need thereof. In one embodiment, the present disclosure provides RNA, oligoribonucleotide, and polyribonucleotide molecules comprising a modified nucleoside, gene therapy vectors comprising same, gene therapy methods and gene transcription silencing methods comprising same. In one embodiment, the mRNA to be administered comprises at least one modified nucleoside.

The polynucleotides encoding peptides described herein can be synthesized by chemical techniques, for example, the phosphotriester method of Matteucci, et al., J. Am. Chem. Soc. 103:3185 (1981). Polynucleotides encoding peptides comprising or consisting of an analog can be made simply by substituting the appropriate and desired nucleic acid base(s) for those that encode the native epitope.

A large number of vectors and host systems suitable for producing and administering a viral epitope peptide described herein are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, pXT1, pSG (Stratagene) pSVK3, pBPV, pMSG, pSVL (Pharmacia); p75.6 (Valentis); pCEP (Invitrogen); pCEI (Epimmune). However, any other plasmid or vector can be used as long as it is replicable and viable in the host.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Thus, the present disclosure is also directed to vectors, and expression vectors useful for the production and administration of the viral epitope peptides described herein, and to host cells comprising such vectors.

Host cells are genetically engineered (transduced or transformed or transfected) with the vectors which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

For expression of the viral epitope peptides described herein, the coding sequence will be provided operably linked start and stop codons, promoter and terminator regions, and in some embodiments, and a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), acid phosphatase, or heat shock proteins, among others. The heterologous structural sequence is assembled in appropriate phase with translation initiation and termination sequences, and in some embodiments, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Yeast, insect or mammalian cell hosts can also be used, employing suitable vectors and control sequences. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. Such promoters can also be derived from viral sources, such as, e.g., human cytomegalovirus (CMV-IE promoter) or herpes simplex virus type-1 (HSV TK promoter). Nucleic acid sequences derived from the SV40 splice, and polyadenylation sites can be used to provide the non-transcribed genetic elements.

Polynucleotides encoding viral epitope peptides described herein can also comprise a ubiquitination signal sequence, and/or a targeting sequence such as an endoplasmic reticulum (ER) signal sequence to facilitate movement of the resulting peptide into the endoplasmic reticulum.

Polynucleotides described herein can be administered and expressed in human cells (e.g., immune cells, including dendritic cells). A human codon usage table can be used to guide the codon choice for each amino acid. Such polynucleotides comprise spacer amino acid residues between epitopes and/or analogs, such as those described above, or can comprise naturally-occurring flanking sequences adjacent to the epitopes and/or analogs (and/or CTL, HTL, and B cell epitopes).

In some embodiments, a viral epitope peptide described herein can also be administered/expressed by viral or bacterial vectors. Examples of expression vectors include attenuated viral hosts, such as vaccinia or fowlpox. As an example of this approach, vaccinia virus is used as a vector to express nucleotide sequences that encode the viral epitope peptides described herein. Vaccinia vectors and methods useful in immunization protocols are described in, e.g., U.S. Pat. No. 4,722,848. Another vector is BCG (Bacille Calmette Guerin). BCG vectors are described by Stover et al., Nature 351:456-460 (1991). A wide variety of other vectors useful for therapeutic administration or immunization of the viral epitope polypeptides described herein, e.g. adeno and adeno-associated virus vectors, retroviral vectors, Salmonella typhi vectors, detoxified anthrax toxin vectors, Sendai virus vectors, poxvirus vectors, canarypox vectors, and fowlpox vectors, and the like, will be apparent to those skilled in the art from the description herein. In some embodiments, the vector is Modified Vaccinia Ankara (VA) (e.g. Bavarian Nordic (MVA-BN)).

Standard regulatory sequences well known to those of skill in the art can be included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. In some embodiments, the promoter is the CMV-IE promoter.

Polynucleotides described herein can comprise one or more synthetic or naturally-occurring introns in the transcribed region. The inclusion of mRNA stabilization sequences and sequences for replication in mammalian cells can also be considered for increasing polynucleotide expression.

In addition, a polynucleotide described herein can comprise immunostimulatory sequences (ISSs or CpGs). These sequences can be included in the vector, outside the polynucleotide coding sequence to enhance immunogenicity.

### Viral Epitopes

Coronaviruses are enveloped positive-stranded RNA viruses that belong to the family Coronaviridae and the order Nidovirales. Coronaviruses frequently infect people around the globe. There are a large number of coronaviruses, most of which circulate among peridomestic animals including pigs, camels, bats and cats. Of the seven coronaviruses identified in human so far, Coronaviruses 229E, NL63 were classified as Group 1 antigenic viruses, OC43 and HKU1 were classified as Group 2 antigenic viruses. They typically infect upper respiratory tract in human and can bring about acute respiratory syndrome and can be fatal. Coronaviruses may be zoonotic in origin. The SARS-CoV, MERS-CoV and 2019 SARS COV-2 have human transmission and infective capability and have caused major public health concern worldwide over a short period within the century. The expansion of genetic diversity among coronaviruses and their consequent ability to cause disease in human beings is mainly achieved through infecting peridomestic animals, which serve as intermediate hosts, nurturing recombination and mutation events. The spike glycoprotein (S glycoprotein), which attaches the virion to the host cell membrane, is postulated to play a dominant role in host range restriction. While SARS-CoV and 2019 SARS CoV-2 infect type 2 pneumocytes and ciliated bronchial epithelial cells utilizing angiotensin converting enzyme 2 (ACE2) as a receptor, MERS-CoV exploits dipeptidyl peptidase 4 (DPP4), a transmembrane glycoprotein, to infect type 2 pneumocytes and unciliated bronchial epithelial cells.

Coronaviruses first replicate in epithelial cells of the respiratory and enteric cells. Human airway epithelial cells facilitate high growth rate for the 2019 SARS CoV-2 virus. Coronavirus infected human beings can present with influenza-like symptoms and can develop pneumonia. Associated symptoms with the disease include cough, fever, dyspnea, myalgia or fatigue. Some human patients present with mild clinical manifestation of the disease. However, the manifestation of the disease in human population can span a wide range from asymptomatic to fatal. In some cases, human coronavirus has an incubation period of 2-4 days; 2019 SARS CoV-2 is estimated to be 3-6 days, and SARS-CoV can be 4-6 days. SARS coronavirus was identified in 2003 and may have originated from an animal reservoir, and first infected humans in Guangdong province in southern China in 2002. Patients presented respiratory distress and diarrhea. MERS-CoV was identified in Saudi Arabia in 2012. Dromedary camels may have been the major reservoirs of MERS-CoV. Typical MERS symptoms include fever, cough, shortness of breath, pneumonia, gastrointestinal symptoms including diarrhea. 2019 SARS CoV-2 is also called SARS CoV-2 or simply CoV-2.

Human-to-human transmission of SARS-CoV occurred after early importation of cases were Toronto in Canada, Hong Kong Special Administrative Region of China, Chinese Taipei, Singapore, and Hanoi in Viet Nam during the global epidemic of 2003; at least four resurgences have since been reported. MERS is reported to have spread to countries, including at least Algeria, Austria, Bahrain, China, Egypt, France, Germany, Greece, Islamic Republic of Iran, Italy, Jordan, Kuwait, Lebanon, Malaysia, the Netherlands, Oman, Philippines, Qatar, Republic of Korea, Kingdom of Saudi Arabia, Thailand, Tunisia, Turkey, United Arab Emirates, United Kingdom, United States, and Yemen during the 2012 outbreak. The 2019 SARS CoV-2 was first identified in Wuhan, China and spread worldwide between December 2019 and early 2020.

As of Mar. 20, 2019, no vaccines had been approved for these viruses. Novel therapeutics against the virus are needed. The present disclosure comprises methods and compositions for developing immunotherapy using subject's own immune cells to activate immune response against the virus.

In one aspect the method comprises one or more of the following:
Analyzing the virus genome sequence to obtain information on potential viral epitopes.
Analyzing a subject's MHC class I and MHC class II expression profiles.
Analyzing the viral sequences in MHC-peptide presentation prediction algorithm implemented in a computer processor wherein the MHC-peptide presentation prediction algorithm implemented in a computer processor has been trained by a machine learning training module that incorporates a large number of characteristics related to the peptide and peptide MHC interactions in order to provide an output of a selection of peptides that are predicted to bind to a certain MHC molecule. In some embodiments, the MHC-peptide presentation predictor is neonmhc2. In some embodiments, a further analysis using MHC-peptide presentation predictor NetMHCpan or NetMHCpan II is performed for comparison. In some embodiments, the MHC-peptide presentation predictor is NetMHCpan. In some embodiments, the MHC-peptide presentation predictor is NetMHCpan II.
Identifying which viral epitopes can bind to an MHC present in the subject.
Ranking aided by a machine learning the viral peptides that bind to the subjects' MHC molecules according to the binding affinities, where higher rank infers higher binding affinity and presentation efficiency.
Select at least one, at least two, at least three or at least four or more viral peptides from the ranked peptides that have high binding affinity to the subject's one or more MHC molecules and prepare a composition. The viral peptides that are selected, taken together may bind to one or more class I MHCs, or a class II MHCs or a mixture of class I and class II MHCs, wherein each of the MHCs is expressed by the subject.

Provided herein an antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 15 or Table 16. Also provided herein is a polynucleotide encoding and antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 14C, Table 15 or Table 16. The antigenic peptide and/or polynucleotide may be recombinant. The antigenic peptide and/or polynucleotide may be isolated or purified. The antigenic peptide may be synthetic or expressed from a polynucleotide.

Also provided herein is an antibody or B cell comprising an antibody that binds to an antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 14C, Table 15 or Table 16.

Also provided herein is a T cell receptor (TCR) or T cell comprising a TCR that that binds an epitope sequence from Table 1A or Table 1B in complex with a corresponding MHC class I molecule according to Table 1A or Table 1B. For example, the TCR can bind to an epitope sequence from column 2 (set 1) of Table 1A in complex with a corresponding MHC class I molecule from column 3 (set 1) in the same row of Table 1A. For example, the TCR can bind to an epitope sequence from column 4 (set 2) of Table 1A in complex with a corresponding MHC class I molecule from column 5 (set 2) in the same row of Table 1A. For example, the TCR can bind to an epitope sequence from column 6 (set 3) of Table 1A in complex with a corresponding MHC class I molecule from column 7 (set 3) in the same row of Table 1A. For example, the TCR can bind to an epitope sequence from column 2 (set 1) of Table 1B in complex with a corresponding MHC class I molecule from column 3 (set 1) in the same row of Table 1B. For example, the TCR can bind to an epitope sequence from column 4 (set 2) of Table 1B in complex with a corresponding MHC class I molecule from column 5 (set 2) in the same row of Table 1B.

Also provided herein is a T cell receptor (TCR) or T cell comprising a TCR that that binds to an epitope sequence from Table 2Ai in complex with a corresponding MHC class II molecule according to Table 2Ai. For example, the TCR can bind to an epitope sequence from column 2 (set 1) of Table 2Ai in complex with a corresponding MHC class II molecule from column 3 (set 1) in the same row of Table 2Ai. For example, the TCR can bind to an epitope sequence from column 4 (set 2) of Table 2Ai in complex with a corresponding MHC class II molecule from column 5 (set 2) in the same row of Table 2Ai. Also provided herein is a T cell receptor (TCR) or T cell comprising a TCR that that binds to an epitope sequence from Table 2Aii in complex with a corresponding MHC class II molecule according to Table 2Aii. For example, the TCR can bind to an epitope sequence from column 2 (set 1) of Table 2Aii in complex with a corresponding MHC class II molecule from column 3 (set 1) in the same row of Table 2Aii.

Provided herein is a method of treating or preventing viral infection in a subject in need thereof comprising administering to the subject an antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 14C, Table 15 or Table 16. Also provided herein is a method of treating or preventing viral infection in a subject in need thereof comprising administering to the subject a polynucleotide encoding and antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 14C, Table 15 or Table 16.

Also provided herein is a method of treating or preventing a viral infection in a subject in need thereof comprising administering to the subject an antibody or B cell comprising an antibody that binds to an antigenic peptide comprising an epitope sequence from Table 1A, Table 1B, Table 1C, Table 2Ai, Table 2Aii, Table 2B, Table 9, Table 10, Table 11, Table 12, Table 14A, Table 14B, Table 14C, Table 15 or Table 16.

Also provided herein is a method of treating or preventing viral infection in a subject in need thereof comprising administering to the subject a T cell receptor (TCR) or T cell comprising a TCR that that binds an epitope sequence from Table 1A, Table 1B, Table 1C, Table 2Ai, Table 2Aii, Table 2B, Table 9, Table 10, Table 11, Table 12, Table 14A, Table 14B, Table 15 or Table 16 in complex with a corresponding MHC class I molecule according to Table 1A, Table 1B, Table 1C, Table 2Ai, Table 2Aii, Table 2B, Table 9, Table 10, Table 11, Table 12, Table 14A, Table 14B, Table 14C, Table 15 or Table 16.

For example, the method can comprise administering to the subject a TCR or T cell comprising a TCR that can bind to an epitope sequence from column 2 (set 1) of Table 1A in complex with a corresponding MHC class I molecule from column 3 (set 1) in the same row of Table 1A. For example, the method can comprise administering to a TCR or T cell comprising a TCR that can bind to an epitope sequence from column 2 (set 1) of Table 1A in complex with a corresponding MHC class I molecule from column 3 (set 1) in the same row of Table 1A to a subject that expresses the corresponding MHC class I molecule from column 3 (set 1). For example, the method can comprise administering to the subject a TCR or T cell comprising a TCR that can bind to an epitope sequence from column 4 (set 2) of Table 1A in complex with a corresponding MHC class I molecule from column 5 (set 2) in the same row of Table 1A. For example, the method can comprise administering to a TCR or T cell comprising a TCR that can bind to an epitope sequence from column 4 (set 2) of Table 1A in complex with a corresponding MHC class I molecule from column 5 (set 2) in the same row of Table 1A to a subject that expresses the corresponding MHC class I molecule from column 5 (set 2). For example, the method can comprise administering to the subject a TCR or T cell comprising a TCR that can bind to an epitope sequence from column 6 (set 3) of Table 1A in complex with a corresponding MHC class I molecule from column 7 (set 3) in the same row of Table 1A. For example, the method can comprise administering to a TCR or T cell comprising a TCR that can bind to an epitope sequence from column 6 (set 3) of Table 1A in complex with a corresponding MHC class I molecule from column 7 (set 3) in the same row of Table 1A to a subject that expresses the corresponding MHC class I molecule from column 7 (set 3).

For example, the method can comprise administering to the subject a TCR or T cell comprising a TCR that can bind to an epitope sequence from column 2 (set 1) of Table 1B in complex with a corresponding MHC class I molecule from column 3 (set 1) in the same row of Table 1B. For example, the method can comprise administering to a TCR or T cell comprising a TCR that can bind to an epitope sequence from column 2 (set 1) of Table 1B in complex with a corresponding MHC class I molecule from column 3 (set 1) in the same row of Table 1B to a subject that expresses the corresponding MHC class I molecule from column 3 (set 1). For example, the method can comprise administering to the subject a TCR or T cell comprising a TCR that can bind to an epitope sequence from column 4 (set 2) of Table 1B in complex with a corresponding MHC class I molecule from column 5 (set 2) in the same row of Table 1B. For example, the method can comprise administering to a TCR or T cell comprising a TCR that can bind to an epitope sequence from column 4 (set 2) of Table 1B in complex with a corresponding MHC class I molecule from column 5 (set 2) in the same row of Table 1B to a subject that expresses the corresponding MHC class I molecule from column 5 (set 2).

For example, the method can comprise administering to the subject a TCR or T cell comprising a TCR that can bind to an epitope sequence from column 2 (set 1) of Table 2Ai in complex with a corresponding MHC class II molecule from column 3 (set 1) in the same row of Table 2Ai. For example, the method can comprise administering to a TCR or T cell comprising a TCR that can bind to an epitope sequence from column 2 (set 1) of Table 2Ai in complex with a corresponding MHC class II molecule from column 3 (set 1) in the same row of Table 2Ai to a subject that expresses the corresponding MHC class II molecule from column 3 (set 1). For example, the method can comprise administering to the subject a TCR or T cell comprising a TCR that can bind to an epitope sequence from column 4 (set 2) of Table 2Ai in complex with a corresponding MHC class II molecule from column 5 (set 2) in the same row of Table 2Ai. For example, the method can comprise administering to a TCR or T cell comprising a TCR that can bind to an epitope sequence from column 4 (set 2) of Table 2Ai in complex with a corresponding MHC class II molecule from column 5 (set 2) in the same row of Table 2Ai to a subject that expresses the corresponding MHC class II molecule from column 5 (set 2). Likewise, the method can comprise administering to the subject a TCR or T cell comprising a TCR that can bind to an epitope sequence from column on the left of Table 2Aii in complex with a corresponding MHC class II molecule from the respective column on the right in the same row of Table 2Aii. A protein encoded by the corresponding allele to the right adjacent column of a peptide in any single row of Table 2Ai or Table 2Aii is an MHC protein that binds to the peptide and is presented to T cells by APCs. A peptide listed on the immediate left column of an HLA allele(s) in each row is matched with the HLA in the row.

TABLE 1A
Peptides and Alleles

| | Set 1 | Set 1 | Set 2 | Set 2 | Set 3 | Set 3 |
|---|---|---|---|---|---|---|
| | Peptide | Allele | Peptide | Allele | Peptide | Allele |
| 1 | AACCHLAKAL | L61 | TPLVPFWI | L47 | FLVLLPLV | L2; L47; L4 |
| 2 | AADLDDFSKQL | L68 | TPLVPFWITIA | L50 | FLYENAFLP | L12; L2; L4 |
| 3 | AADPAMHAAS | L68 | TPLVPFWITIAY | L31 | FMGRIRSVY | L14; L43; L69 |
| 4 | AAGLEAPFLYL | L67 | TPNNTDFSRV | L51 | FNATRFASV | L3; L20; L73 |
| 5 | AAISDYDY | L54 | TPRDLGAC | L51 | FNIDYDCVSF | L43; L71; L72 |
| 6 | AAISDYDYYR | L15 | TPSFKKGAKLL | L39 | FPDLNGDVVAI | L68; L50; L49 |
| 7 | AAITILDGI | L10 | TPSKLIEYT | L49 | FPFNKWGKA | L50; L51; L46 |
| 8 | AAITILDGISQY | L14 | TPVYSFLPGVY | L31 | FPLCANGQVF | L31; L50; L32 |
| 9 | AAKKNNLPFK | L13 | TQEVFAQV | L48 | FPLNSIIKT | L50; L31; L47 |
| 10 | AALALLLLDR | L7 | TQFNYYKK | L24 | FPLNSIIKTI | L47; L50; L49 |
| 11 | AALCTFLL | L74 | TQGNFGDQEL | L34 | FRIDGDMVP | L66; L35; L34 |
| 12 | AALQIPFA | L50 | TQGNFGDQELI | L34 | FRKSNLKPF | L66; L30; L58 |
| 13 | AALQPEEEQ | L67 | TQHGKEDLKF | L26 | FRLTLGVYDY | L66; L30; L12 |
| 14 | AAMQRKLEK | L7 | TQLPPAYTNSF | L8 | FRSSVLHST | L66; L35; L64 |
| 15 | AANTVIWDYKR | L15 | TQLSTDTGV | L24 | FSALEPLV | L48; L47; L70 |
| 16 | AARDLICAQ | L43 | TQLYLGGMSYY | L14 | FSKLINIIIW | L52; L69; L70 |
| 17 | AARDLICAQK | L13 | TQQLIRAA | L48 | FSSLPSYAAF | L59; L60; L43 |
| 18 | AARVVRSIFSR | L15 | TQRNFYEPQI | L48 | FSTVFPPTSF | L43; L60; L59 |
| 19 | AARYMRSLK | L13 | TQSLLIVNN | L24 | FTSLEIPRR | L19; L17; L21 |
| 20 | AASGNLLLD | L69 | TQYEYGTEDDY | L14 | FTYICGFIQQK | L7; L6; L19 |
| 21 | AASGNLLLDK | L7 | TRAGCLIGA | L30 | FVDGVPFVVS | L68; L5; L67 |
| 22 | AAVDALCEKAL | L67 | TRCLAVHEC | L35 | FVDSDVET | L63; L68; L62 |
| 23 | AAVINGDRWF | L52 | TRCNLGGAV | L35 | FVENPDILRV | L20; L5; L63 |
| 24 | AAVNSVPW | L54 | TRDIASTD | L65 | FVENPDILRVY | L14; L1; L31 |
| 25 | AAVYRINW | L54 | TRDIASTDT | L35 | FVETVKGL | L63; L68; L60 |
| 26 | AAYSRYRIGNY | L14 | TRDIASTDTCF | L34 | FVETVKGLDY | L1; L12; L18 |
| 27 | ACLVGLMWL | L4 | TREVGFVV | L35 | FVFKNIDGYF | L11; L58; L12 |
| 28 | ACPLIAAVI | L57 | TRFASVYAWNR | L30 | FVKATCEF | L58; L59; L43 |
| 29 | ACTDDNALA | L42 | TRFQTLLALH | L30 | FVLAAVYR | L17; L15; L21 |
| 30 | ACTDDNALAYY | L14 | TRFQTLLALHR | L30 | FVRIIMRL | L74; L59; L23 |
| 31 | ACVEEVTTTL | L36 | TRGATVVIG | L65 | FVRIIMRLW | L52; L49; L10 |
| 32 | ACYNGSPSGV | L3 | TRKLMPVCV | L64 | FVTVYSHLLL | L74; L60; L61 |
| 33 | ACYNGSPSGVY | L14 | TRLQSLENVAF | L30 | FYKGVITH | L71; L66; L72 |
| 34 | ADAQSFLN | L33 | TRNPANNAA | L35 | FYLCFLAFLLF | L9; L12; L8 |
| 35 | ADAQSFLNGF | L33 | TRNPAWRKAVF | L65 | FYPKLQSS | L71; L72; L66 |
| 36 | ADDLNQLT | L33 | TRQVVNVVT | L35 | FYPKLQSSQ | L71; L72; L66 |
| 37 | ADKFPVLHD | L65 | TRSTNSRI | L35 | FYTSKTTVA | L72; L71; L46 |
| 38 | ADLKSFDL | L33 | TRYVDNNFC | L34 | FYVLGLAA | L50; L72; L71 |
| 39 | ADLVYALR | L33 | TSALLAGTI | L55 | FYVLPNDDTL | L67; L9; L71 |
| 40 | ADLVYALRH | L33 | TSAMQTML | L74 | FYYLGTGPEA | L50; L71; L72 |
| 41 | ADLYKLMGH | L33 | TSDLATNNLVV | L68 | GADLKSFDL | L68; L63; L60 |
| 42 | ADLYKLMGHF | L33 | TSFGPLVRKIF | L52 | GADPIHSLRV | L18; L63; L5 |
| 43 | ADNKFALTC | L33 | TSGDGTTS | L65 | GAPTKVTF | L23; L63; L57 |
| 44 | ADPAMHAA | L33 | TSHTVMPL | L57 | GAWNIGEQK | L13; L7; L19 |
| 45 | ADPIHSLR | L33 | TSITSAVLQ | L19 | GCDGGSLY | L1; L18; L14 |
| 46 | ADPIHSLRV | L33 | TSLEIPRRNV | L13 | GCDGGSLYV | L18; L2; L45 |
| 47 | ADQAMTQM | L33 | TSLLVLVQ | L48 | GDDYVYLPY | L18; L1; L65 |
| 48 | ADQAMTQMY | L14 | TSNPTTFH | L43 | GDFKLNEEI | L37; L24; L34 |
| 49 | ADQLTPTW | L33 | TSNPTTFHLD | L65 | GDFSHSQL | L25; L33; L37 |
| 50 | ADSFVIRG | L33 | TSPDVDLGD | L65 | GEAANFCALI | L45; L36; L41 |
| 51 | ADSIIIGG | L33 | TSQWLTNI | L48 | GEAVKTQFN | L45; L36; L40 |
| 52 | ADSIIIGGA | L33 | TSQWLTNIF | L53 | GEIKDATP | L38; L46; L37 |
| 53 | ADSKIVQLS | L33 | TSSIVITSG | L65 | GEIPVAYRKVLL | L36; L40; L41 |
| 54 | ADSTLIGD | L33 | TSTDVVYRA | L20 | GELGDVRETM | L36; L41; L37 |
| 55 | ADVFHLYL | L33 | TSWQTGDFVK | L7 | GEVITFDNLKTL | L36; L37; L41 |
| 56 | ADVFHLYLQ | L33 | TTAAKLMV | L73 | GEVPVSIINN | L36; L37; L41 |
| 57 | ADVTKIKP | L33 | TTADIVVFDEI | L20 | GEYTFEKGD | L45; L46; L37 |
| 58 | ADYNYKLPD | L65 | TTAPAICHD | L20 | GFELTSMKY | L12; L18; L1 |
| 59 | ADYSVLYN | L33 | TTAPAICHDGK | L19 | GFFKEGSSV | L72; L24; L71 |
| 60 | AEASKKPRQK | L46 | TTAYANSV | L20 | GFMGRIRSVY | L71; L14; L72 |
| 61 | AECTIFKD | L33 | TTDNTFVS | L1 | GFNEKTHVQL | L8; L71; L62 |
| 62 | AEETRKLM | L45 | TTDPSFLGRYM | L1 | GFNFSQIL | L62; L72; L71 |
| 63 | AEGSRGGSQ | L40 | TTEMLAKALR | L19 | GFSTGVNL | L72; L71; L62 |
| 64 | AEIRASAN | L45 | TTETAHSC | L18 | GHMLDMYSV | L35; L28; L34 |
| 65 | AELAKNVS | L46 | TTEVVGDIILK | L7 | GHNLAKHCL | L35; L34; L28 |
| 66 | AENVTGLFKDC | L40 | TTFHLDGEV | L20 | GHTDLMAAY | L27; L12; L14 |
| 67 | AESHVDTD | L33 | TTFHLDGEVI | L20 | GHVETFYPKL | L28; L34; L35 |
| 68 | AEVAVKMFD | L41 | TTFHLDGEVITF | L52 | GIATVREV | L48; L3; L64 |
| 69 | AFACPDGVKHVY | L14 | TTKGGRFV | L70 | GIFGADPIHSL | L16; L2; L10 |
| 70 | AFASEAARV | L24 | TTLPVNVAFEL | L52 | GIIAMSAF | L43; L26; L59 |
| | | | W | | | |
| 71 | AFDIYNDK | L62 | TTNIVTRCLNR | L15 | GINASVVNI | L24; L3; L4 |
| 72 | AFDIYNDKV | L62 | TTRQVVNV | L48 | GLDSLDTY | L18; L1; L14 |
| 73 | AFDKSAFVN | L62 | TTSPISEHD | L20 | GLEAPFLY | L1; L18; L12 |
| 74 | AFEHIVYG | L62 | TTTIQTIV | L48 | GLIAIVMVTI | L3; L24; L2 |
| 75 | AFFGMSRI | L48 | TTTLNGLW | L52 | GLNDNLLEIL | L4; L2; L3 |
| 76 | AFGEYSHVVA | L42 | TTTNIVTRCL | L20 | GLNLEEAARY | L14; L12; L18 |
| 77 | AFHTPAFDK | L7 | TTTYKLNVGDY | L14 | GLPGTILRT | L24; L5; L2 |
| 78 | AFKQIVESC | L13 | TTYKLNVGD | L19 | GLVASIKNF | L10; L27; L26 |
| 79 | AFLLFLVLIM | L71 | TTYPGQGLN | L14 | GMPSYCTGY | L26; L27; L12 |
| 80 | AFLPFAMGIIAM | L71 | TVAAFHQEC | L20 | GNFGDQELI | L34; L24; L36 |
| 81 | AFVNLKQLP | L46 | TVAYFNMV | L20 | GPFVDRQTA | L51; L50; L46 |
| 82 | AFVNLKQLPFFY | L12 | TVCTVCGMW | L10 | GQVDLFRNA | L24; L13; L46 |
| 83 | AGDSGFAAYSRY | L18 | TVDNINLH | L1 | GRCDIKDL | L66; L25; L64 |
| 84 | AGFAKFLK | L7 | TVDNINLHT | L68 | GRVDGQVDLF | L30; L34; L65 |
| 85 | AGGIVAIV | L48 | TVDSSQGSEYD | L18 | GSSVELKHF | L53; L52; L26 |
| | | | Y | | | |
| 86 | AGLEAPFLYL | L67 | TVFFDGRV | L20 | GTAVLRQW | L52; L54; L55 |
| 87 | AGLEAPFLYLY | L14 | TVHTANKW | L54 | GTDLEGNF | L1; L62; L63 |
| 88 | AGSYKDWSY | L14 | TVIEVQGYKSV | L10 | GTDTTITV | L18; L63; L68 |
| 89 | AHISTIGVCSM | L34 | TVKGLDYKA | L51 | GTGPEAGLPY | L18; L12; L7 |
| 90 | AHVASCDAIM | L28 | TVKNYFITD | L13 | GTGTSTDVVYR | L7; L15; L19 |
| 91 | AICHDGKAH | L43 | TVKSVGKFCL | L39 | GTITVEEL | L67; L55; L54 |
| 92 | AIDAYPLTKH | L6 | TVLKKCKSA | L23 | GTITVEELK | L7; L13; L19 |
| 93 | AIDGGVTRDI | L68 | TVLKKCKSAF | L23 | GTLMIERF | L54; L52; L53 |
| 94 | AIFYLITPVHV | L20 | TVLSFCAFAV | L4 | GTLSYEQF | L54; L53; L52 |
| 95 | AIKCVPQAD | L43 | TVMFLARGI | L55 | GTLSYEQFK | L7; L6; L13 |
| 96 | AILTALRLC | L14 | TVRTNVYLAVF | L52 | GTNLPLQL | L73; L55; L60 |
| 97 | AILTALRLCAY | L14 | TVSALVYDNK | L7 | GTSKFYGGW | L10; L54; L52 |
| 98 | AIMQLFFSYF | L12 | TVVIGTSK | L19 | GVAGALNK | L7; L6; L19 |
| 99 | AISSVLNDI | L24 | TVVIGTSKFY | L14 | GVEHVTFFIY | L12; L18; L1 |
| 100 | AIVFITLCFTLK | L7 | TVVVNAANV | L20 | GVLTESNKK | L7; L13; L6 |
| 101 | AKCWTETDL | L44 | TVVVNAANVY | L14 | GVYYHKNNK | L7; L6; L13 |
| 102 | AKEIKESVQTF | L27 | TVYEKLKPVL | L20 | GWTAGAAAY | L12; L14; L27 |
| 103 | AKFLKTNCC | L27 | TVYSDVENP | L49 | GYFCTCYF | L72; L71; L9 |
| 104 | AKFLKTNCCRF | L27 | TVYSHLLLVAA | L51 | GYLPQNAV | L48; L71; L72 |
| 105 | AKHCLHVVG | L44 | TWICLLQFAY | L12 | GYQPYRVV | L72; L71; L64 |
| 106 | AKKNNLPFK | L13 | TWLDMVDTSL | L8 | HAEETRKLM | L32; L63; L49 |
| 107 | AKKPTETIC | L27 | TWLTYTGAI | L8 | HDVSSAINR | L19; L17; L15 |
| 108 | AKNRARTV | L48 | TWLTYTGAIKL | L8 | HEHEIAWYTE | L37; L46; L29 |
| 109 | AKNVSLDNVL | L44 | TYERHSLSH | L71 | HEVLLAPLLSA | L46; L41; L38 |
| 110 | AKPFLNKVV | L64 | TYGQQFGPTYL | L9 | HFAWWTAF | L72; L71; L62 |
| 111 | AKRFKESPF | L27 | TYKLNVGDYF | L9 | HFDGQQGEV | L62; L68; L63 |
| 112 | AKSASVYY | L27 | TYNCCDDDY | L14 | HFISNSWL | L72; L71; L62 |
| 113 | AKVGILCIM | L44 | TYTGAIKLD | L9 | HFPREGVF | L62; L72; L71 |
| 114 | ALAPNMMVTN | L3 | TYYLFDESGEF | L9 | HFPREGVFV | L62; L71; L72 |
| 115 | ALGGSVAI | L24 | VAAIFYLI | L47 | HFVCNLLL | L62; L71; L72 |
| 116 | ALGGSVAIKI | L24 | VAAIVFITLCF | L52 | HFVCNLLLLF | L8; L12; L9 |
| 117 | ALGKLQDV | L24 | VAALTNNVA | L50 | HFVNLDNL | L71; L62; L72 |
| 118 | ALHRSYLTP | L46 | VADAVIKTLQ | L63 | HFVNLDNLR | L17; L15; L19 |
| 119 | ALITLATC | L3 | VAEWFLAYIL | L67 | HFYSKWYIR | L17; L15; L21 |
| 120 | ALKGGKIVNNW | L16 | VAFELWAKRN | L69 | HGHVMVEL | L23; L25; L59 |
| 121 | ALLADKFPVLH | L6 | VAGDSGFAA | L50 | HHANEYRLYL | L34; L28; L35 |
| 122 | ALLAVFQSASK | L6 | VAGFAKFLK | L7 | HHSIGFDYV | L34; L28; L35 |
| 123 | ALLLLDRLNQL | L4 | VAGVSICST | L43 | HIDAYKTF | L63; L62; L68 |
| 124 | ALLTLQQIELK | L6 | VAIDYKHYTP | L67 | HLAKALNDF | L26; L11; L10 |
| 125 | ALNKATNNA | L3 | VAIHADQLTPT | L52 | HLYLQYIRK | L6; L13; L7 |
| | | | W | | | |
| 126 | ALRANSAV | L3 | VAKSHNIALI | L70 | HPLADNKF | L32; L31; L47 |
| 127 | ALTGIAVEQ | L24 | VAKSHNIALIW | L52 | HPNPKGFCD | L32; L49; L39 |
| 128 | ALWEIQQVVD | L2 | VALLAVFQSA | L50 | HQKLLKSI | L48; L25; L23 |
| 129 | ALWEIQQVVDA | L2 | VAMKYNYEPL | L67 | HQSDIEVT | L24; L35; L44 |
| 130 | AMDEFIERYKL | L5 | VANGDSEVVLK | L7 | HSDKFTDGV | L18; L1; L63 |
| 131 | AMPNMLRI | L48 | VANYQKVGM | L58 | HSMQNCVLK | L7; L13; L19 |
| 132 | AMQMAYRF | L33 | VAPGTAVLRQW | L52 | HTDLMAAY | L18; L1; L68 |
| 133 | AMRNAGIVGV | L3 | VARDLSLQFK | L13 | HTIDGSSGVV | L11; L20; L10 |
| 134 | AMRPNFTI | L48 | VASCDAIMTR | L19 | HTPAFDKSAF | L57; L11; L10 |
| 135 | AMSAFAMMF | L12 | VASPNECNQM | L70 | HTQVVDMSMTY | L18; L14; L1 |
| 136 | ANFCALIL | L73 | VAVLYQDVN | L67 | HTSPDVDL | L53; L20; L54 |
| 137 | ANFCALILA | L46 | VAYSNNSIAI | L47 | HVDILGPL | L68; L63; L62 |
| 138 | ANFCALILAY | L12 | VCRFDTRV | L48 | HVDTDLTKPY | L18; L1; L14 |
| 139 | ANGDSEVVLKK | L7 | VCRFDTRVL | L39 | HVSGTNGTK | L19; L7; L6 |
| 140 | ANGGKGFCK | L7 | VCRLMKTI | L48 | HVYQLRAR | L17; L15; L21 |
| 141 | ANLDSCKRV | L4 | VCSVIDLL | L62 | IADTTDAV | L68; L63; L67 |
| 142 | ANLDSCKRVL | L65 | VCTNYMPYF | L9 | IAMSAFAM | L59; L61; L70 |
| 143 | ANLGERVRQA | L65 | VCTNYMPYFF | L9 | IASFRLFAR | L17; L19; L15 |
| 144 | ANNAAIVLQL | L73 | VCVETKAI | L48 | IAVLDMCASL | L67; L31; L60 |
| 145 | ANNCTFEYV | L73 | VDALCEKAL | L33 | IAYIICISTK | L19; L47; L43 |
| 146 | ANNTKGSL | L23 | VDDIVKTD | L33 | ICISTKHFY | L14; L12; L58 |
| 147 | ANQFNSAIGK | L7 | VDGQVDLF | L33 | IDYVPLKSA | L46; L51; L43 |
| 148 | ANQVIVNNL | L72 | VDGVVQQL | L33 | IEELFYSY | L29; L18; L1 |
| 149 | ANRNRFLYI | L73 | VDNSSLTI | L48 | IEELFYSYA | L38; L42; L46 |
| 150 | ANSIVCRF | L33 | VDSDLNDF | L33 | IELKFNPPA | L46; L38; L42 |
| 151 | ANSIVCRFD | L65 | VDSVTVKN | L33 | IELKFNPPAL | L36; L37; L29 |
| 152 | ANYQKVGM | L33 | VDSYYSLL | L33 | IELSLIDFYL | L40; L36; L41 |
| 153 | ANYQKVGMQKY | L14 | VDSYYSLLM | L33 | IERFVSLAI | L45; L48; L46 |
| 154 | APAHISTIG | L22 | VDTDLTKPY | L43 | IEYTDFATSA | L46; L38; L42 |
| 155 | APFLYLYALVY | L31 | VDTSLSGF | L33 | IFFASFYY | L12; L71; L72 |
| 156 | APHGVVFLH | L56 | VDTVRTNVY | L29 | IFYLITPVH | L72; L71; L48 |
| 157 | APHGVVFLHVT | L56 | VDTVSALV | L33 | IGIVNNTVY | L26; L27; L29 |
| 158 | APHGVVFLHVTY | L56 | VDVELFENK | L7 | IGVTQNVLY | L12; L52; L14 |
| 159 | APISAMVRMY | L31 | VDVVNFNL | L33 | IHADQLTPTW | L28; L52; L54 |
| 160 | APKEIIFLEG | L51 | VEAEVQID | L29 | IIAYTMSL | L59; L60; L61 |
| 161 | APLIELCV | L47 | VEAEVQIDR | L19 | IKDATPSDF | L62; L27; L63 |
| 162 | APLIELCVDEA | L50 | VEAFEYYHT | L38 | IKDLPKEI | L68; L62; L63 |
| 163 | APLLSAGIFGA | L50 | VEAFEYYHTT | L38 | IKNFKSVLY | L27; L12; L18 |
| 164 | APLTVFFD | L65 | VECTTIVN | L29 | IKWDLLKY | L27; L18; L66 |
| 165 | APRITFGGP | L51 | VECTTIVNGV | L45 | ILAYCNKTV | L3; L2; L4 |
| 166 | APRITFGGPSD | L22 | VEEAKTVLK | L38 | ILHCANFNV | L4; L2; L5 |
| 167 | APRTLLTKGTL | L22 | VEEVLSEA | L38 | ILLLDQALV | L2; L4; L5 |
| 168 | APTHLSVDT | L56 | VEFLRDGW | L29 | ILPDPSKPS | L57; L43; L5 |
| 169 | APTKVTFGD | L56 | VEGFNCYF | L33 | ILQKEKVNI | L24; L2; L4 |
| 170 | APTLVPQEHY | L31 | VEIIKSQD | L45 | ILSDDAVVC | L2; L26; L4 |
| 171 | AQAAGTDTT | L44 | VEIIKSQDLSV | L38 | ILSPLYAFA | L3; L2; L5 |
| 172 | AQFAPSASAFF | L27 | VEKGIYQTS | L45 | ILTLTRAL | L23; L59; L57 |
| 173 | AQLPAPRT | L48 | VELGTEVN | L29 | IMSDRDLY | L18; L14; L1 |
| 174 | AQLPAPRTLL | L44 | VENPTIQK | L29 | INIIIWELL | L73; L9; L8 |
| 175 | AQLPAPRTLLTK | L7 | VENPTIQKDV | L45 | INIIIWFLLL | L73; L8; L67 |
| 176 | AQSFLNGFAV | L24 | VENPTIQKDVL | L36 | IPARARVEC | L56; L22; L39 |
| 177 | AQTGSSKCV | L24 | VESDDYIA | L38 | IPCSVCLSGL | L56; L22; L39 |
| 178 | AQVDVVNFNL | L44 | VESSSKLW | L40 | IPCTCGKQA | L51; L50; L39 |
| 179 | AQVLSEMV | L48 | VETKAIVS | L29 | IPIGAGICA | L51; L50; L22 |
| 180 | AQYELKHG | L48 | VEVEKGVLPQL | L36 | IPLMYKGL | L39; L47; L23 |
| 181 | ARARVECF | L35 | VEVQPQLEMEL | L36 | IPMDSTVKN | L49; L31; L32 |
| 182 | ARARVECFDK | L30 | VEVVDKYF | L29 | IPYNSVTSS | L51; L47; L50 |
| 183 | ARDGCVPLNII | L34 | VEWKFYDA | L46 | IQASLPFGW | L52; L16; L54 |
| 184 | ARDLICAQK | L30 | VEYCPIFFITG | L46 | IQLSSYSL | L44; L35; L25 |
| 185 | ARDLSLQFK | L30 | VFAFPFTI | L9 | IRGWIFGTTL | L35; L30; L66 |
| 186 | ARDLSLQFKR | L30 | VFAFPFTIYSL | L8 | IRKLHDEL | L25; L66; L67 |
| 187 | ARFPKSDGT | L30 | VFAQVKQI | L48 | ISAARQGF | L54; L53; L52 |
| 188 | ARFPKSDGTGT | L30 | VFDEISMA | L62 | ISAMVRMY | L55; L70; L52 |
| 189 | ARFYFYTS | L65 | VFDEISMATN | L62 | ISQYSLRLI | L55; L48; L73 |
| 190 | ARGIVFMC | L65 | VFDGKSKCE | L62 | ISSDVLVNN | L53; L54; L52 |
| 191 | ARHINAQVAK | L30 | VFDKNLYD | L62 | ISTKHFYWFF | L53; L54; L52 |
| 192 | ARIVYTACSH | L30 | VFDKNLYDK | L62 | ISTSHKLV | L48; L70; L73 |
| 193 | ARLRAKHY | L66 | VFDKNLYDKL | L62 | ITEGSVKGL | L63; L60; L39 |
| 194 | ARLRAKHYV | L30 | VFITLCFTLK | L13 | ITFELDER | L21; L15; L17 |
| 195 | ARLTPCGTGT | L30 | VFKNIDGYFK | L13 | ITISSFKW | L54; L52; L53 |
| 196 | ARLYYDSM | L66 | VFKNIDGYFKI | L8 | ITPVHVMSK | L57; L6; L7 |
| 197 | ARQGFVDSD | L65 | VFLGIITTVA | L8 | ITSAVLQSGF | L52; L54; L53 |
| 198 | ARSEDKRAK | L30 | VFLHVTYVP | L8 | IVAAIVFI | L20; L47; L74 |
| 199 | ARSVSPKLFIR | L30 | VFNATRFA | L71 | IVDEPEEHVQI | L5; L68; L63 |
| 200 | ARTAPHGHV | L30 | VFNATRFASV | L71 | IVFDGKSKC | L16; L11; L14 |
| 201 | ARTVYDDGAR | L30 | VFNATRFASVY | L71 | IVMVTIML | L74; L68; L60 |
| 202 | ARTVYDDGARR | L30 | VFNGVSFS | L62 | IVNGVRRSFY | L14; L12; L18 |
| 203 | ARVECFDKFK | L30 | VFNGVSFST | L8 | IVNNATNVV | L58; L51; L73 |
| 204 | ARVECFDKFKV | L30 | VFPLNSIIK | L62 | IVVFDEISM | L31; L32; L67 |
| 205 | ARVVRSIF | L66 | VFSAVGNICY | L12 | IYDEPTTT | L62; L63; L68 |
| 206 | ARVVRSIFSR | L30 | VFTGYRVTK | L13 | IYLYLTFY | L71; L72; L12 |
| 207 | ASALGKLQD | L65 | VFVLWAHGFEL | L71 | IYSKHTPINL | L9; L8; L66 |
| 208 | ASANIGCNH | L43 | VGDFKLNEEI | L68 | KAGQKTYER | L21; L15; L17 |
| 209 | ASCTLSEQL | L53 | VGEIPVAYR | L15 | KAKKGAWNI | L55; L53; L70 |
| 210 | ASDTYACWHH | L18 | VGHTDLMAAY | L14 | KASMPTTI | L55; L70; L54 |
| 211 | ASEAARVV | L48 | VGKPRPPLNR | L15 | KCVPQADVEW | L54; L52; L16 |
| 212 | ASEFSSLPS | L1 | VGPEHSLAE | L57 | KCYGVSPTK | L13; L6; L7 |
| 213 | ASEYTGNYQC | L18 | VGPKQASL | L57 | KDFGGFNF | L33; L54; L65 |
| 214 | ASFSASTS | L55 | VGYLQPRT | L48 | KDGHVETF | L33; L54; L65 |
| 215 | ASFSTFKC | L54 | VGYQPYRVVVL | L67 | KDQVILLNK | L13; L7; L6 |
| 216 | ASFYYVWKS | L69 | VHFISNSW | L28 | KEGFFTYIC | L42; L38; L37 |
| 217 | ASHMYCSF | L43 | VHFISNSWLM | L28 | KEIIFLEG | L45; L37; L46 |
| 218 | ASIKNFKSVLY | L18 | VHFVCNLLLL | L34 | KEIIFLEGE | L46; L37; L42 |
| 219 | ASIKNFKSVLYY | L18 | VHFVCNLLLLF | L34 | KEIIFLEGETL | L36; L37; L41 |
| 220 | ASIVAGGI | L55 | VHNQDVNLH | L34 | KEILVTYN | L33; L45; L29 |
| 221 | ASIVAGGIVA | L42 | VIDLLLDD | L68 | KEITVATSRTL | L36; L37; L41 |
| 222 | ASKIITLK | L13 | VIGAVILR | L21 | KEKVNINI | L45; L33; L37 |
| 223 | ASKIITLKKR | L15 | VIHFGAGSD | L43 | KFCLEASF | L62; L71; L72 |
| 224 | ASKIITLKKRW | L52 | VIKVCEFQF | L26 | KFKVNSTL | L71; L72; L62 |
| 225 | ASKILGLPT | L43 | VILLNKHI | L48 | KFNPPALQD | L65; L13; L8 |
| 226 | ASLVLARK | L7 | VISTSHKL | L60 | KFPVLHDI | L62; L71; L72 |
| 227 | ASPNECNQM | L57 | VIWDYKRD | L43 | KGEDIQLL | L63; L55; L65 |
| 228 | ASQGLVASIK | L7 | VIYLYLTFYL | L67 | KGLPWNVVR | L15; L21; L7 |
| 229 | ASQSIIAYT | L14 | VKDCVVLH | L62 | KGLPWNVVRI | L8; L4; L16 |
| 230 | ASQSIIAYTM | L55 | VKGLDYKAF | L27 | KGPKVKYL | L57; L64; L66 |
| 231 | ASRELKVT | L43 | VKIGPERTC | L27 | KGTHHWLLL | L73; L69; L8 |
| 232 | ASSRSSSRSR | L15 | VKLQNNEL | L44 | KGTLEPEYF | L54; L52; L9 |
| 233 | ASTEKSNII | L55 | VKNGSIHL | L66 | KHFYWFFSNY | L27; L14; L16 |
| 234 | ASTEKSNIIR | L7 | VKNGSIHLYF | L27 | KHLIPLMYK | L6; L13; L7 |
| 235 | ASTSAFVET | L73 | VKNKCVNF | L27 | KHYTPSFKK | L6; L7; L13 |
| 236 | ASVVNIQK | L7 | VKPGGTSSG | L57 | KIAEIPKEE | L16; L43; L3 |
| 237 | ASVYAWNR | L15 | VKPTVVVN | L66 | KIFVDGVPFV | L13; L2; L4 |
| 238 | ASVYAWNRK | L7 | VKQGDDYVYL | L44 | KIKACVEEV | L13; L3; L58 |
| 239 | ASVYYSQLM | L54 | VKRHTFSNY | L27 | KIVDEPEEH | L16; L43; L54 |
| 240 | ASWFTALTQ | L18 | VKSREETGL | L44 | KIYSKHTPI | L16; L24; L6 |
| 241 | ASWVMRIM | L55 | VKSREETGLL | L44 | KKAGGTTEM | L27; L44; L66 |
| 242 | ASYQTQTN | L48 | VKSVGKFCL | L44 | KKQQTVTL | L44; L27; L66 |
| 243 | ATAEAELAKN | L10 | VKYLYFIKGL | L27 | KLALGGSVA | L51; L3; L46 |
| 244 | ATALLTLQQI | L53 | VLAAVYRI | L24 | KLASHMYCSF | L16; L43; L26 |
| 245 | ATAQEAYEQ | L53 | VLCNSQTSLR | L21 | KLDNDALNNI | L5; L2; L18 |
| 246 | ATEGALNT | L18 | VLDMCASLK | L1 | KLFDRYFK | L13; L6; L21 |
| 247 | ATEGALNTPK | L7 | VLGSLAATVR | L21 | KLIEYTDFA | L2; L5; L3 |
| 248 | ATEVPANST | L18 | VLHDIGNPKA | L2 | KLINIIIW | L16; L54; L69 |
| 249 | ATEVPANSTV | L18 | VLHSTQDLFL | L57 | KLKDCVMYA | L3; L13; L2 |
| 250 | ATHGLAAVN | L14 | VLKKCKSA | L23 | KLLKSIAAT | L16; L2; L4 |
| 251 | ATHSDKFTD | L53 | VLKLKVDT | L23 | KLMPVCVET | L2; L5; L4 |
| 252 | ATKMSECVL | L70 | VLKTGDLQPL | L3 | KLMPVCVETK | L6; L16; L13 |
| 253 | ATKYLVQQE | L13 | VLLFLAFVVFL | L4 | KLNDLCFTN | L16; L4; L21 |
| 254 | ATNKATYK | L7 | VLLILMTA | L23 | KLNVGDYFV | L2; L4; L5 |
| 255 | ATNYDLSV | L73 | VLLPLVSS | L23 | KLPDDFTGCV | L5; L2; L57 |
| 256 | ATREAVGTN | L10 | VLLRKNGNK | L6 | KLRGTAVMSL | L16; L3; L2 |
| 257 | ATREAVGTNL | L53 | VLMDGSIIQ | L2 | KLRSDVLLP | L6; L16; L13 |
| 258 | ATRFASVYA | L13 | VLMSNLGMPSY | L12 | KMADQAMTQM | L18; L14; L16 |
| | | | | | Y | |
| 259 | ATRFASVYAW | L52 | VLPFNDGV | L57 | KNLREFVFK | L13; L6; L7 |
| 260 | ATRGATVV | L70 | VLQKAAITIL | L74 | KNNLPFKL | L73; L55; L33 |
| 261 | ATRVECTTI | L53 | VLQVRDVL | L23 | KNSIDAFKL | L55; L53; L73 |
| 262 | ATSACVLAA | L42 | VLSDRELHLSW | L16 | KPLEFGATS | L51; L56; L50 |
| 263 | ATSRTLSYYK | L7 | VLSEARQHLK | L6 | KPRPPLNRN | L51; L22; L39 |
| 264 | ATTAYANSVF | L52 | VLSTFISAAR | L21 | KPRQKRTA | L51; L39; L65 |
| 265 | ATVCGPKK | L7 | VLSVNPYV | L2 | KPRQKRTAT | L22; L51; L39 |
| 266 | ATYYLFDES | L67 | VLTAVVIPTK | L6 | KPTVVVNAA | L51; L50; L56 |
| 267 | AVDAAKAYKDY | L18 | VLTAVVIPTKK | L6 | KQEILGTVSW | L16; L52; L18 |
| 268 | AVDALCEK | L7 | VLVPHVGEIPV | L2 | KQQTVTLL | L48; L33; L44 |
| 269 | AVDALCEKA | L5 | VLYENQKLIA | L2 | KQVEQKIAEI | L44; L3; L24 |
| 270 | AVDALCEKALKY | L18 | VLYNSASFSTF | L26 | KQVVSDIDY | L26; L27; L14 |
| 271 | AVDINKLCE | L18 | VLYNSASFSTFK | L6 | KRGDKSVY | L65; L66; L14 |
| 272 | AVFISPYNS | L7 | VLYQDVNCTEV | L2 | KRSDARTA | L65; L66; L55 |
| 273 | AVGTNLPLQL | L6 | VLYYQNNV | L48 | KRTIKGTHHW | L16; L54; L52 |
| 274 | AVHECFVK | L7 | VMAYITGGV | L3 | KRVDFCGKGY | L30; L66; L14 |
| 275 | AVHECFVKRV | L3 | VMCGGSLYV | L2 | KRWQLALSK | L30; L13; L6 |
| 276 | AVILRGHL | L74 | VMCGGSLYVK | L7 | KSASVYYSQ | L54; L55; L53 |
| 277 | AVILRGHLRI | L24 | VMFTPLVPFW | L52 | KSHFAIGL | L55; L73; L54 |
| 278 | AVINGDRW | L54 | VMHANYIFWR | L21 | KSHFAIGLALY | L14; L18; L52 |
| 279 | AVINGDRWFL | L16 | VMLTNDNTSR | L15 | KSVGKFCL | L65; L54; L55 |
| 280 | AVITREVGFV | L3 | VMLTNDNTSRY | L14 | KSWMESEF | L53; L55; L54 |
| 281 | AVKRTIKGTHHW | L52 | VMPLSAPTLV | L5 | KSYVHVVD | L55; L65; L54 |
| 282 | AVKTQFNY | L14 | VMRIMTWL | L48 | KTDGTLMIER | L21; L18; L15 |
| 283 | AVLQSGFRKM | L14 | VMYASAVVLL | L74 | KTFPPTEP | L54; L55; L73 |
| 284 | AVSKGFFK | L7 | VNAANVYL | L74 | KTIQPRVEKK | L13; L7; L6 |
| 285 | AVTAYNGY | L14 | VNAANVYLK | L7 | KTLLSLREVR | L21; L15; L52 |
| 286 | AVTAYNGYL | L74 | VNASSSEAF | L9 | KTQSLLIV | L73; L70; L18 |
| 287 | AVVLLILM | L74 | VNEFYAYL | L62 | KTQSLLIVN | L53; L13; L21 |
| 288 | AVVLLILMT | L7 | VNINIVGDF | L9 | KTTEVVGDI | L55; L53; L54 |
| 289 | AVVLLILMTA | L51 | VNKGEDIQL | L39 | KTTEVVGDII | L53; L54; L55 |
| 290 | AWPLIVTA | L62 | VNLHSSRLSF | L12 | KTYERHSLSH | L16; L6; L18 |
| 291 | AWPLIVTALR | L21 | VNLKQLPFFY | L12 | KVAGFAKFLK | L7; L6; L21 |
| 292 | AWQPGVAMPNL | L14 | VNLKQLPFFYY | L12 | KVEGCMVQV | L63; L5; L13 |
| | Y | | | | | |
| 293 | AWRKAVFISP | L42 | VNLVAVPTGY | L12 | KVFTTVDNI | L16; L54; L24 |
| 294 | AWYTERSEKSY | L14 | VNNATNKATY | L14 | KVGGNYNY | L54; L14; L18 |
| 295 | AYEQAVAN | L71 | VNNATNVV | L48 | KVKPTVVVNA | L13; L51; L3 |
| 296 | AYFNMVYMPAS | L9 | VNSFSGYLK | L7 | KVLNEKCSAY | L14; L16; L6 |
| | W | | | | | |
| 297 | AYSRYRIGNY | L14 | VNSTLEQYVF | L9 | KVNINIVGD | L16; L21; L65 |
| 298 | AYTKRNVIP | L42 | VNSVLLFL | L73 | KVNSTLEQYVF | L16; L54; L52 |
| 299 | AYTKRNVIPTI | L9 | VNSVLLFLAF | L12 | KVTLADAGF | L54; L52; L53 |
| 300 | AYTNSFTR | L15 | VNVVTTKI | L48 | KVTSAMQTM | L54; L16; L53 |
| 301 | AYTNSFTRG | L9 | VPANSTVLS | L56 | KVVSTTTNI | L54; L16; L24 |
| 302 | AYTNSFTRGVY | L14 | VPAQEKNF | L39 | KWDLIISDMY | L18; L14; L1 |
| 303 | AYTVELGTEV | L72 | VPATVSVSSP | L51 | KWGKARLY | L55; L14; L18 |
| 304 | AYYFMRFR | L15 | VPEVKILNN | L49 | KWPWYIWL | L57; L62; L71 |
| 305 | AYYNTTKGG | L72 | VPFVVSTG | L47 | KWYIRVGAR | L21; L15; L6 |
| 306 | CAFAVDAA | L50 | VPFWITIAYII | L47 | KYAISAKNR | L15; L21; L17 |
| 307 | CAFAVDAAK | L19 | VPGLPGTI | L47 | KYCALAPNM | L71; L72; L9 |
| 308 | CAKEIKES | L43 | VPGLPGTILR | L21 | KYFKNHTSP | L8; L46; L71 |
| 309 | CALAPNMMV | L47 | VPHVGEIPVAY | L31 | KYKGIKIQE | L13; L8; L15 |
| 310 | CALDPLSET | L59 | VPINTNSSP | L51 | KYNYEPLTQ | L9; L8; L65 |
| 311 | CAMRPNFTI | L47 | VPLKSATCI | L47 | KYTQLCQYL | L9; L8; L64 |
| 312 | CANDPVGFTLK | L6 | VPLNIIPLTT | L50 | KYVQIPTTC | L8; L9; L66 |
| 313 | CANFNVLF | L58 | VPLNIIPLTTAA | L50 | KYVRNLQHR | L15; L17; L8 |
| 314 | CAQVLSEM | L59 | VPQEHYVRIT | L39 | KYWDQTYHP | L8; L9; L66 |
| 315 | CATTRQVV | L70 | VPVAIHAD | L51 | LAAVYRINW | L49; L52; L69 |
| 316 | CAYCCNIVN | L69 | VPVSIINNTVY | L31 | LAFVVFLLVT | L31; L47; L50 |
| 317 | CAYCCNIVNV | L69 | VPYNMRVIH | L47 | LAGTITSGW | L49; L52; L54 |
| 318 | CAYWVPRASA | L50 | VQAGNVQLRV | L24 | LAVFDKNL | L60; L61; L74 |
| 319 | CCSLSHRF | L33 | VQELYSPIF | L27 | LAYILFTR | L47; L17; L59 |
| 320 | CDAMRNAGI | L33 | VQELYSPIFL | L68 | LDDDSQQTV | L5; L68; L63 |
| 321 | CDGTTFTY | L65 | VQHMVVKAA | L51 | LDDFSKQL | L33; L68; L62 |
| 322 | CDIRQLLF | L33 | VQIGEYTFEK | L7 | LDGEVITF | L33; L65; L29 |
| 323 | CDIRQLLFV | L73 | VQIPCTCGK | L13 | LDNVLSTF | L33; L59; L43 |
| 324 | CDLKGKYV | L33 | VQIPTTCA | L48 | LEAPFLYLYA | L46; L42; L38 |
| 325 | CDQLREPML | L33 | VQLHNDILLAK | L6 | LEAPFLYLYAL | L41; L40; L36 |
| 326 | CDTLKEIL | L33 | VQLRVIGHSM | L14 | LECIKDLLA | L38; L46; L42 |
| 327 | CDTLKEILV | L33 | VQLSEISM | L44 | LEMKSEKQV | L45; L38; L37 |
| 328 | CEEMLDNRATL | L36 | VQLSLPVLQ | L44 | LENVAFNV | L33; L29; L45 |
| 329 | CEFQFCNDPF | L29 | VQLSLPVLQVR | L15 | LEYHDVRV | L45; L48; L37 |
| 330 | CEFQFCNDPFL | L36 | VQMAPISAMVR | L21 | LEYHDVRVVL | L36; L37; L41 |
| 331 | CEIVGGQIVTC | L36 | VQMLSDTLK | L7 | LFFSYFAVH | L71; L72; L12 |
| 332 | CEKALKYL | L37 | VQPTESIV | L48 | LFLPFFSNVTW | L52; L9; L8 |
| 333 | CFDKFKVNS | L62 | VQQESPFV | L48 | LFLTWICL | L71; L72; L62 |
| 334 | CFKMFYKGV | L13 | VQSKMSDV | L48 | LFLTWICLLQF | L8; L9; L12 |
| 335 | CFLGYFCTC | L8 | VQSTQWSLFF | L9 | LFLVLIML | L71; L72; L62 |
| 336 | CFLGYFCTCYF | L8 | VQTFFKLV | L48 | LFLVLIMLI | L47; L9; L8 |
| 337 | CFVDDIVKT | L8 | VQTFFKLVNK | L7 | LFVAAIFY | L72; L71; L12 |
| 338 | CGFIQQKLAL | L65 | VQVTCGTTTL | L44 | LGDIAARDL | L68; L63; L62 |
| 339 | CGPKKSTNL | L57 | VRDLPQGFSAL | L68 | LGDVRETM | L63; L62; L68 |
| 340 | CGYLPQNAV | L48 | VRFPNITNLC | L30 | LGLPTQTV | L47; L48; L25 |
| 341 | CHATREAV | L28 | VRKIFVDGV | L64 | LHCANFNVL | L35; L28; L34 |
| 342 | CHDGKAHFP | L34 | VRLQAGNAT | L30 | LHSTQDLFL | L28; L34; L35 |
| 343 | CHIDHPNPK | L28 | VRMYIFFASF | L30 | LIAAVITR | L19; L21; L17 |
| 344 | CHIDHPNPKGF | L8 | VRTNVYLAVF | L66 | LIDSYFVV | L68; L63; L5 |
| 345 | CHNKCAYWV | L35 | VRVLQKAA | L25 | LIRKSNHNF | L58; L26; L52 |
| 346 | CIKDLLARA | L3 | VSAKPPPGD | L43 | LITPVHVM | L59; L61; L60 |
| 347 | CINANQVIV | L73 | VSALVYDNK | L7 | LIVAAIVFI | L20; L47; L5 |
| 348 | CIRCLWSTK | L13 | VSELLTPLG | L1 | LIVGVALL | L74; L60; L61 |
| 349 | CISTKHFY | L14 | VSGNCDVVI | L53 | LIVNSVLL | L60; L74; L61 |
| 350 | CISTKHFYW | L54 | VSGTNGTKRF | L52 | LKEILVTY | L27; L18; L1 |
| 351 | CKDGHVETFY | L27 | VSIINNTVYTK | L7 | LKRGDKSVY | L27; L26; L43 |
| 352 | CLAVHECFV | L2 | VSKVVKVT | L43 | LKVGGSCVL | L44; L27; L61 |
| 353 | CLAVHECFVKR | L19 | VSKVVKVTIDY | L43 | LKVPATVSV | L50; L51; L64 |
| 354 | CLAYYFMR | L21 | VSLAIDAYPL | L55 | LLALHRSYL | L74; L3; L4 |
| 355 | CLFLLPSLA | L3 | VSLLSVLLS | L52 | LLDQALVSD | L5; L68; L1 |
| 356 | CLFWNCNVDR | L21 | VSLLSVLLSM | L52 | LLEIKDTEKY | L1; L18; L12 |
| 357 | CLGDIAAR | L21 | VSMTKTSV | L48 | LLFVVEVV | L3; L23; L47 |
| 358 | CLLNRYFR | L21 | VSTIQRKYK | L13 | LLKSIAATR | L21; L15; L17 |
| 359 | CLNRVCTNY | L26 | VSTQEFRY | L54 | LLLDQALV | L2; L4; L5 |
| 360 | CLSGLDSLDTY | L14 | VTCLAYYF | L62 | LLLSVCLGSL | L4; L2; L5 |
| 361 | CLVGLMWL | L74 | VTFFPDLNG | L7 | LLPPKNSI | L57; L5; L23 |
| 362 | CNVKTTEV | L48 | VTFGDDTVIEV | L20 | LLPSLATV | L5; L57; L3 |
| 363 | CPACHNSEV | L22 | VTFQSAVK | L7 | LLPSLATVAY | L12; L57; L5 |
| 364 | CPDGVKHV | L47 | VTFQSAVKRTI | L52 | LLSAGIFGA | L3; L2; L4 |
| 365 | CPIFFITG | L50 | VTHSKGLYRK | L7 | LLSVCLGSL | L57; L4; L3 |
| 366 | CPNFVFPL | L22 | VTIDYTEISFM | L11 | LLSVLQQL | L4; L59; L23 |
| 367 | CPNFVFPLN | L50 | VTKGKAKK | L13 | LLTKSSEY | L26; L43; L1 |
| 368 | CQAVTANV | L48 | VTKNSKVQIGEY | L14 | LLVTLAIL | L74; L23; L59 |
| 369 | CQEPKLGSL | L44 | VTLACFVL | L55 | LMNVLTLV | L48; L62; L47 |
| 370 | CQPILLLD | L48 | VTLADAGF | L54 | LMWLIINL | L47; L62; L74 |
| 371 | CQVHGNAHV | L24 | VTLADAGFI | L55 | LNDILSRL | L63; L68; L62 |
| 372 | CQYLNTLTLAV | L48 | VTLADAGFIKQY | L14 | LNLEEAARY | L12; L14; L31 |
| 373 | CRFDTRVLSN | L65 | VTLIGEAV | L48 | LNRYFRLTL | L65; L73; L22 |
| 374 | CRFDTRVLSNL | L30 | VTLIGEAVK | L7 | LNVGDYFVL | L65; L67; L32 |
| 375 | CRFQEKDED | L65 | VTLKQGEI | L55 | LPDDFTGCV | L32; L49; L47 |
| 376 | CRFVTDTPK | L30 | VTLKQGEIK | L7 | LPFKLTCAT | L50; L32; L51 |
| 377 | CRHHANEY | L66 | VTQNVLYENQK | L7 | LPGVYSVIY | L31; L65; L49 |
| 378 | CRKVQHMVV | L25 | VTQQLIRAA | L51 | LPGVYSVIYL | L56; L39; L22 |
| 379 | CRMNSRNY | L65 | VTRCLNRV | L48 | LPIDKCSRI | L47; L49; L32 |
| 380 | CRMNSRNYI | L34 | VTRCLNRVC | L52 | LPLTQYNRY | L31; L49; L12 |
| 381 | CSFYPPDED | L65 | VTRDIAST | L43 | LPLVSSQCV | L47; L50; L51 |
| 382 | CSLSHRFY | L55 | VTSAMQTM | L70 | LPNNTASW | L49; L50; L56 |
| 383 | CSQHTMLV | L48 | VTSNYSGVV | L70 | LPQLEQPYV | L47; L50; L51 |
| 384 | CSQHTMLVK | L7 | VTTTLEETK | L7 | LPQLEQPYVF | L31; L32; L39 |
| 385 | CSRIIPAR | L48 | VTYKLDGVV | L73 | LPTGTLLV | L47; L50; L51 |
| 386 | CTDDNALA | L1 | VTYNCCDDDY | L14 | LPVLQVRDVL | L31; L22; L32 |
| 387 | CTFEYVSQPFL | L20 | VVDGCNSSTCM | L63 | LPWNVVRI | L47; L50; L49 |
| 388 | CTLKSFTVEK | L7 | VVDMSMTYG | L68 | LPWNVVRIK | L31; L47; L32 |
| 389 | CTQHQPYVV | L73 | VVFVLWAH | L59 | LPWNVVRIKI | L47; L50; L39 |
| 390 | CTTIVNGVRR | L19 | VVGPNVNK | L7 | LPWNVVRIKIV | L50; L39; L47 |
| 391 | CVCSVIDL | L74 | VVIGIVNN | L74 | LPYPDPSR | L47; L50; L51 |
| 392 | CVDEAGSKS | L63 | VVIGIVNNT | L20 | LQAIASEF | L26; L59; L27 |
| 393 | CVDTVRTNVYL | L68 | VVIGIVNNTV | L20 | LQSINFVRI | L24; L48; L34 |
| 394 | CVEYCPIFF | L62 | VVIPTKKAG | L43 | LRIAGHHL | L66; L35; L25 |
| 395 | CVIAWNSNNL | L10 | VVKAALLA | L51 | LSDRELHLS | L1; L53; L18 |
| 396 | CVLGQSKR | L17 | VVKAALLAD | L51 | LSFKELLV | L73; L48; L47 |
| 397 | CVLSGHNL | L60 | VVLHSYFTSDY | L14 | LSKGRLIIR | L15; L17; L21 |
| 398 | CVRGTTVL | L59 | VVLKKLKKSL | L23 | LSPRWYFYY | L1; L12; L18 |
| 399 | CVVADAVIKTL | L10 | VVLKTGDL | L74 | LSTDTGVEH | L43; L61; L59 |
| 400 | CVVLHSYF | L74 | VVNARLRAKHY | L14 | LSYFIASFR | L19; L15; L17 |
| 401 | CWHHSIGFDY | L14 | VVQQLPETYF | L12 | LTAFGLVAEW | L52; L10; L54 |
| 402 | CYDHVIST | L62 | VVREFLTRN | L43 | LTALRLCAY | L1; L59; L18 |
| 403 | CYDHVISTS | L62 | VVRIKIVQ | L23 | LTENKYSQL | L1; L63; L60 |
| 404 | CYDTNVLE | L62 | VVSDIDYV | L20 | LTENLLLY | L1; L18; L12 |
| 405 | CYDTNVLEG | L62 | VVSTGYHF | L54 | LTENLLLYI | L1; L18; L73 |
| 406 | CYDYCIPY | L62 | VVTCLAYY | L14 | LTESNKKFL | L1; L63; L60 |
| 407 | CYDYCIPYN | L62 | VVTTVMFL | L74 | LTGHMLDMY | L1; L18; L12 |
| 408 | CYTPSKLI | L9 | VVVNAANVYL | L20 | LTILTSLLV | L73; L1; L18 |
| 409 | DAALALLLLDR | L19 | VVVNAANVYLK | L7 | LTKEGATTC | L55; L53; L52 |
| 410 | DADSTLIGD | L49 | VWTLMNVLTL | L9 | LTLQQIELK | L6; L7; L52 |
| 411 | DAFKLNIK | L47 | VYAADPAMH | L9 | LTLQQIELKF | L52; L12; L53 |
| 412 | DAGFIKQY | L70 | VYANLGER | L17 | LTNDNTSR | L21; L15; L17 |
| 413 | DALCEKALK | L19 | VYCCREHEH | L72 | LTNMFTPL | L59; L61; L60 |
| 414 | DAMRNAGI | L47 | VYCFLGYFC | L9 | LTPVVQTI | L47; L55; L57 |
| 415 | DAMRNAGIV | L47 | VYDDGARR | L62 | LTRNPAWRK | L13; L6; L7 |
| 416 | DAQGMDNL | L74 | VYDNKLKA | L62 | LVAEWFLAYI | L20; L3; L4 |
| 417 | DATPSDFV | L47 | VYDPLQPE | L62 | LVASIKNF | L74; L52; L58 |
| 418 | DATPSDFVRA | L50 | VYDPLQPELDSF | L9 | LVASIKNFK | L6; L7; L19 |
| 419 | DAVIKTLQP | L49 | VYDYLVST | L62 | LVAVPTGY | L26; L43; L1 |
| 420 | DAVNLLTN | L47 | VYFASTEKS | L9 | LVDFQVTI | L68; L63; L62 |
| 421 | DAVNLLTNMF | L10 | VYFASTEKSNI | L9 | LVDLPIGI | L68; L62; L63 |
| 422 | DAVVCFNSTY | L10 | VYFLQSINFVR | L15 | LVEVEKGVL | L68; L63; L1 |
| 423 | DAYKTFPPT | L47 | VYIGDPAQ | L71 | LVFLFVAAIF | L31; L12; L52 |
| 424 | DAYNMMIS | L47 | VYKQFDTYNLW | L9 | LVKQGDDYVY | L14; L26; L52 |
| 425 | DAYNMMISAGF | L11 | VYLAVFDKNLY | L12 | LVKQLSSNF | L26; L58; L52 |
| 426 | DAYVNTFSS | L47 | VYNPFMID | L66 | LVPFWITIAY | L12; L26; L6 |
| 427 | DAYYRARAG | L25 | VYNPFMIDVQ | L9 | LWLDDVVYC | L31; L4; L8 |
| 428 | DCATVHTANK | L19 | VYNPFMIDVQQ | L9 | LYDANYFL | L62; L68; L63 |
| | | | W | | | |
| 429 | DCSARHINA | L25 | VYQCAMRPN | L72 | LYFDKAGQKTY | L71; L14; L12 |
| 430 | DCVVLHSY | L29 | VYRAFDIY | L66 | LYLDAYNM | L71; L72; L66 |
| 431 | DCVVLHSYF | L29 | VYSVIYLYLT | L9 | LYPTLNISD | L71; L72; L9 |
| 432 | DDFTGCVI | L25 | VYYSQLMCQ | L72 | MADSNGTI | L68; L63; L47 |
| 433 | DDFVEIIKS | L29 | VYYTSNPTTFHL | L9 | MADSNGTIT | L63; L68; L32 |
| 434 | DDKKIKAC | L23 | WCKDGHVET | L43 | MAFPSGKV | L47; L70; L69 |
| 435 | DDNALAYY | L29 | WDLIISDM | L33 | MCDIRQLL | L62; L63; L68 |
| 436 | DDNLIDSY | L29 | WDLLKYDF | L33 | MDEFIERY | L18; L1; L29 |
| 437 | DDNLIDSYF | L29 | WDLTAFGL | L33 | MEVTPSGTWL | L36; L37; L41 |
| 438 | DDQIGYYR | L17 | WEIVKFISTC | L45 | MFVFLVLL | L62; L71; L72 |
| 439 | DDQIGYYRR | L17 | WEPEFYEA | L42 | MFYKGVITH | L71; L72; L17 |
| 440 | DDTLRVEA | L25 | WESGVKDCVV | L38 | MHAASGNLL | L34; L28; L35 |
| 441 | DDTVIEVQGY | L29 | WESGVKDCVVL | L36 | MHAASGNLLL | L28; L34; L35 |
| 442 | DDVVYCPRH | L29 | WEVGKPRP | L46 | MISAGFSL | L60; L61; L28 |
| 443 | DDYQGKPL | L25 | WEVGKPRPPL | L36 | MLDMYSVM | L63; L68; L62 |
| 444 | DDYVYLPY | L29 | WFHAIHVSG | L71 | MLTNDNTSRY | L14; L12; L18 |
| 445 | DEAGSKSPIQY | L29 | WFLAYILFT | L12 | MLVKQGDDY | L26; L14; L12 |
| 446 | DEDDNLID | L29 | WFLNRFTTT | L8 | MPVCVETKA | L50; L51; L32 |
| 447 | DEDDNLIDS | L29 | WHNMLKTV | L48 | MPYFFTLLLQL | L31; L50; L47 |
| 448 | DEDDNLIDSYF | L41 | WHNMLKTVY | L27 | MSAFAMMFV | L73; L20; L70 |
| 449 | DEDDSEPV | L29 | WHTNCYDYCI | L34 | MSKFPLKLR | L15; L17; L21 |
| 450 | DEDEEEGD | L29 | WICLLQFAY | L12 | MSYEDQDALF | L54; L53; L52 |
| 451 | DEEQPMEID | L29 | WKSYVHVVD | L44 | MTQMYKQAR | L17; L15; L21 |
| 452 | DEFIERYK | L29 | WLCWKCRSK | L13 | MVTNNTFTLK | L6; L7; L19 |
| 453 | DEFIERYKLEGY | L29 | WLDDVVYCPR | L21 | MVYMPASWVM | L21; L19; L15 |
| | | | | | R | |
| 454 | DEGNCDTL | L29 | WLDDVVYCPRH | L5 | MYKGLPWNV | L13; L64; L3 |
| 455 | DEISMATN | L29 | WLDDVVYCPRH | L5 | NADIVEEA | L68; L67; L63 |
| | | | V | | | |
| 456 | DELGTDPY | L29 | WLDMVDTSLS | L1 | NADLYKLM | L63; L68; L62 |
| 457 | DELTGHMLDM | L29 | WLGFIAGLI | L24 | NAGDYILAN | L67; L69; L43 |
| 458 | DELTGHMLDMY | L29 | WLKQLIKVT | L3 | NAGIVGVL | L67; L70; L74 |
| 459 | DEMIAQYT | L29 | WLKQLIKVTLV | L3 | NALDQAISMW | L10; L49; L52 |
| 460 | DEMIAQYTS | L29 | WLLWPVTLA | L2 | NAVASKILGL | L31; L49; L10 |
| 461 | DEPEEHVQ | L29 | WLSYFIASFR | L21 | NCTEVPVAI | L35; L20; L34 |
| 462 | DEPEEHVQIH | L29 | WMVMFTPLV | L48 | NDFNLVAM | L29; L33; L25 |
| 463 | DEPTTTTS | L29 | WNLVIGFLFL | L73 | NDNLLEIL | L33; L25; L29 |
| 464 | DERIDKVLN | L29 | WNVKDFMSL | L32 | NEFYAYLRK | L41; L40; L29 |
| 465 | DETQALPQR | L29 | WNVVRIKIV | L69 | NEKTHVQLSL | L37; L41; L36 |
| 466 | DEVRQIAPG | L29 | WPLIVTALRA | L50 | NENGTITDA | L42; L38; L46 |
| 467 | DEWSMATY | L29 | WPQIAQFAP | L50 | NETLVTMPL | L37; L29; L41 |
| 468 | DFAVSKGFF | L9 | WPQIAQFAPSA | L50 | NEYRLYLDAY | L29; L41; L40 |
| 469 | DFCGKGYHL | L8 | WPVTLACFV | L47 | NFKSVLYY | L72; L71; L12 |
| 470 | DFGDFIQTT | L17 | WPVTLACFVLA | L50 | NFNVLFSTV | L62; L71; L72 |
| 471 | DFIDTKRGV | L11 | WPWYIWLGFI | L47 | NFSKLINII | L8; L34; L9 |
| 472 | DFIPMDSTV | L71 | WPWYIWLGFIA | L50 | NFYGPFVDR | L17; L19; L15 |
| 473 | DFLEYHDVR | L17 | WQTGDFVKA | L24 | NGDFLHFL | L68; L63; L62 |
| 474 | DFLHFLPR | L17 | WRVYSTGSNV | L30 | NGLMLLEI | L25; L47; L48 |
| 475 | DFLHFLPRVF | L9 | WSFNPETNIL | L60 | NGYPNMFITR | L19; L17; L15 |
| 476 | DFQENWNTK | L17 | WSLFFFLYE | L73 | NHDLYCQVH | L35; L34; L28 |
| 477 | DFVEIIKS | L29 | WSYSGQSTQ | L55 | NHTSPDVDL | L28; L35; L34 |
| 478 | DGADVTKIK | L19 | WTAFVTNV | L20 | NIALIWNVK | L19; L7; L6 |
| 479 | DGCVPLNI | L47 | WTAFVTNVNA | L20 | NIIPLTTA | L3; L25; L23 |
| 480 | DGCVPLNII | L47 | WTAGAAAYYV | L20 | NINLHTQV | L25; L23; L48 |
| 481 | DGDMVPHI | L47 | WTIEYPIIG | L69 | NIVNVSLVK | L19; L7; L6 |
| 482 | DGGSLYVNK | L19 | WTLMNVLTLVY | L52 | NKGAGGHSY | L27; L66; L14 |
| 483 | DGHVETFY | L29 | WVATEGAL | L74 | NLAKHCLHVV | L3; L2; L4 |
| 484 | DGIIWVAT | L25 | WVLNNDYY | L14 | NLIDSYFVVKR | L19; L17; L21 |
| 485 | DGKMKDLSP | L23 | WYDFGDFI | L62 | NLIIKNLSK | L6; L23; L7 |
| 486 | DGKSKCEE | L23 | WYDFVENP | L62 | NLLEILQK | L6; L7; L4 |
| 487 | DGTLMIERF | L29 | WYIRVGAR | L17 | NLLKDCPAV | L4; L2; L5 |
| 488 | DGVDVELF | L29 | WYIRVGARK | L8 | NLLLQYGSF | L10; L27; L12 |
| 489 | DGVKHVYQLR | L19 | WYIWLGFIA | L50 | NLSDRVVFVL | L4; L3; L2 |
| 490 | DGVPFVVSTGY | L11 | WYTERSEKSY | L14 | NLWNTFTR | L21; L17; L19 |
| 491 | DGVVCTEI | L25 | YAADPAMHAAS | L67 | NLYKMQRML | L64; L23; L74 |
| 492 | DGWEIVKFI | L47 | YAAFATAQ | L59 | NNDYYRSL | L68; L62; L63 |
| 493 | DGYVMHANYI | L47 | YAAFATAQEAY | L31 | NNLDKSAGF | L8; L12; L11 |
| 494 | DHCGETSW | L28 | YAAVINGDR | L19 | NNLNRGMVL | L25; L23; L71 |
| 495 | DHIGTRNPA | L28 | YADSFVIRGD | L49 | NPDILRVY | L31; L49; L1 |
| 496 | DHSSSSDNIALL | L34 | YAFASEAA | L50 | NPFMIDVQQW | L49; L31; L41 |
| 497 | DHVISTSH | L28 | YAFEHIVYGD | L49 | NQKLIANQF | L26; L27; L58 |
| 498 | DIAANTVIWDY | L11 | YAFEHIVYGDF | L31 | NQMCLSTL | L25; L48; L35 |
| 499 | DIAKKPTET | L23 | YALVYFLQSI | L47 | NRATLQAI | L25; L35; L64 |
| 500 | DIDYVPLKS | L1 | YANGGKGFC | L58 | NRNYVFTGY | L30; L66; L65 |
| 501 | DIGNPKAIK | L19 | YANGGKGFCKL | L60 | NRPQIGVV | L66; L64; L25 |
| 502 | DILGPLSA | L25 | YASAVVLLILM | L31 | NRQFHQKL | L25; L35; L66 |
| 503 | DINGNLHPD | L11 | YASQGLVASI | L20 | NRVCGVSAA | L25; L35; L30 |
| 504 | DINKLCEEM | L29 | YATHSDKFTD | L49 | NSFSGYLK | L19; L55; L7 |
| 505 | DIPGIPKD | L11 | YAWNRKRIS | L69 | NSFTRGVY | L14; L1; L70 |
| 506 | DIPGIPKDMTY | L11 | YAYLRKHFS | L23 | NSPNLAWPL | L57; L67; L66 |
| 507 | DIPIGAGIC | L11 | YCALAPNMM | L28 | NSRNYIAQV | L13; L70; L48 |
| 508 | DIQLLKSA | L23 | YCPACHNSE | L57 | NSSPDDQIGYY | L1; L14; L18 |
| 509 | DIRQLLFV | L23 | YCPACHNSEV | L57 | NSTNVTIATY | L14; L43; L10 |
| 510 | DISGINASVV | L20 | YCPIFFITG | L57 | NSVPWDTIANY | L52; L14; L11 |
| 511 | DISTEIYQA | L11 | YCPIFFITGNTL | L57 | NTFSSTFNV | L20; L70; L19 |
| 512 | DITPCSFGGV | L11 | YCRHGTCER | L43 | NTVIWDYKR | L17; L19; L15 |
| 513 | DIVEEAKK | L19 | YDANYFLCW | L49 | NVAKYTQL | L74; L25; L56 |
| 514 | DIVEEAKKVK | L19 | YDDGARRVW | L49 | NVGDYFVL | L67; L66; L65 |
| 515 | DIVKTDGTLM | L11 | YDHVISTSH | L43 | NVLSTFISA | L50; L51; L17 |
| 516 | DIVVFDEI | L47 | YDKLVSSFL | L33 | NVLTLVYK | L7; L17; L6 |
| 517 | DKAGQKTY | L27 | YDNKLKAH | L33 | NVPMEKLKTL | L57; L10; L5 |
| 518 | DKAGQKTYER | L19 | YDPKTKNV | L33 | NVSLVKPSF | L31; L39; L12 |
| 519 | DKKIKACV | L23 | YDSMSYED | L33 | NVYADSFVI | L49; L10; L20 |
| 520 | DKNTQEVF | L29 | YDYVIFTQ | L33 | NVYLAVFDK | L7; L19; L6 |
| 521 | DKVEAEVQI | L44 | YDYYRYNL | L33 | NWLKQLIKV | L4; L8; L25 |
| 522 | DKYVRNLQH | L25 | YDYYRYNLP | L33 | NWYDFGDF | L9; L71; L62 |
| 523 | DLATNNLVVM | L11 | YEAMYTPHTVL | L36 | NWYDFGDFI | L34; L8; L9 |
| 524 | DLICAQKF | L11 | YECDIPIGAGI | L45 | NYIAQVDVVNF | L8; L9; L71 |
| 525 | DLKGKYVQ | L23 | YECLYRNRD | L45 | NYIFWRNTN | L72; L8; L71 |
| 526 | DLKPVSEE | L23 | YEDFQENWN | L45 | NYKLPDDF | L62; L9; L71 |
| 527 | DLLIRKSN | L23 | YEDLLIRK | L45 | NYQKVGMQKY | L8; L14; L12 |
| 528 | DLLIRKSNH | L23 | YEDLLIRKSN | L41 | PASWVMRIM | L70; L32; L69 |
| 529 | DLNDFVSD | L23 | YEGNSPFHP | L38 | PAYTNSFTR | L19; L17; L15 |
| 530 | DLNGNWYDF | L11 | YEGNSPFHPL | L36 | PEAGLPYGA | L46; L42; L38 |
| 531 | DLPIGINITR | L19 | YELKHGTFT | L45 | PETTADIVV | L38; L37; L36 |
| 532 | DLPIGINITRF | L11 | YELKHGTFTC | L38 | PETTADIVVF | L29; L41; L40 |
| 533 | DLPKEITV | L23 | YELKHGTFTCA | L46 | PGIPKDMTY | L43; L26; L14 |
| 534 | DLPKEITVA | L11 | YENFNQHEVLL | L36 | PGLPGTILR | L15; L21; L17 |
| 535 | DLSVVNAR | L17 | YEPLTQDH | L33 | PHGHVMVEL | L35; L34; L28 |
| 536 | DLSVVSKVVK | L19 | YEPQIITT | L29 | PLADNKFAL | L4; L2; L16 |
| 537 | DLTAFGLVAEW | L10 | YEQAVANGD | L38 | PLHGTILTR | L21; L19; L17 |
| 538 | DLYCQVHGN | L11 | YEQFKKGVQ | L46 | PLQPELDSF | L9; L26; L16 |
| 539 | DMCASLKEL | L25 | YEQFKKGVQIP | L38 | PPIKDFGGF | L49; L11; L10 |
| 540 | DMFLGTCR | L17 | YEQYIKWP | L38 | PPTSFGPLV | L50; L51; L47 |
| 541 | DMYSVMLTN | L47 | YERHSLSH | L29 | PQNAVVKIY | L26; L14; L27 |
| 542 | DNGPQNQR | L17 | YERHSLSHFV | L45 | PRPPLNRNY | L64; L65; L66 |
| 543 | DNIALLVQ | L25 | YFDCYDGG | L62 | PTITQMNLKY | L11; L12; L18 |
| 544 | DNLACEDL | L25 | YFDCYDGGC | L62 | PTLVPQEHY | L52; L12; L16 |
| 545 | DNLKTLLSLR | L17 | YFDCYDGGCI | L62 | PTMCDIRQL | L55; L53; L10 |
| 546 | DNQDLNGNW | L49 | YFDKAGQK | L62 | PTSFGPLVR | L19; L7; L21 |
| 547 | DNSSLTIKK | L19 | YFDKAGQKT | L62 | PVAYRKVLL | L23; L20; L74 |
| 548 | DNVTDFNAI | L10 | YFFTLLLQ | L48 | PVLDWLEEK | L7; L6; L13 |
| 549 | DNYYKKDNSY | L11 | YFFTLLLQLC | L12 | PVSELLTPL | L20; L67; L56 |
| 550 | DPAQLPAP | L50 | YFGLFCLLNR | L17 | PVYSFLPGVY | L14; L11; L12 |
| 551 | DPAQLPAPRTL | L22 | YFIASFRLFAR | L17 | PWDTIANY | L1; L18; L62 |
| 552 | DPIHSLRV | L47 | YFIKGLNNLNR | L17 | PYFFTLLL | L9; L71; L72 |
| 553 | DPKTKNVT | L23 | YFKNHTSP | L71 | PYIKWDLLKY | L12; L8; L14 |
| 554 | DPKTKNVTK | L23 | YFLQSINFVR | L17 | PYPDPSRI | L9; L8; L62 |
| 555 | DPLSETKC | L47 | YFLQSINFVRI | L8 | QAAGTDTTI | L32; L49; L67 |
| 556 | DPNFKDQVILL | L31 | YFRLTLGV | L48 | QADVEWKF | L63; L68; L62 |
| 557 | DPQTLEILD | L32 | YFRLTLGVYDY | L12 | QADVEWKFY | L1; L18; L70 |
| 558 | DPQTLEILDI | L47 | YFTQSRNLQEF | L9 | QAPTHLSV | L57; L47; L70 |
| 559 | DPSRILGA | L50 | YFYTSKTTVAS | L71 | QECVRGTTVL | L36; L40; L37 |
| 560 | DPVGFTLK | L47 | YFYTSKTTVASL | L71 | QEEVQELYSP | L42; L38; L46 |
| 561 | DPYEDFQE | L47 | YGADLKSFDL | L44 | QEFRYMNSQ | L46; L29; L45 |
| 562 | DPYEDFQENW | L49 | YGDFSHSQ | L68 | QEFRYMNSQGL | L40; L36; L41 |
| 563 | DQALVSDV | L25 | YGDSATLP | L63 | QEILGTVSWNL | L40; L41; L36 |
| 564 | DQDALFAY | L1 | YGFQPTNGV | L47 | QEKNFTTAP | L46; L42; L38 |
| 565 | DQFKHLIP | L25 | YGFQPTNGVGY | L27 | QELIRQGTD | L40; L33; L46 |
| 566 | DQLREPML | L25 | YHDVRVVLDF | L34 | QESPFVMMSA | L42; L46; L38 |
| 567 | DQLTPTWR | L17 | YHDVRVVLDFI | L34 | QEVFAQVKQI | L41; L45; L40 |
| 568 | DQVILLNK | L25 | YHTTDPSF | L28 | QFAYANRNR | L17; L15; L21 |
| 569 | DQVILLNKH | L29 | YHYQECVR | L28 | QHEETIYNL | L34; L35; L28 |
| 570 | DRCILHCA | L25 | YICGDSTEC | L43 | QHEETIYNLL | L34; L28; L35 |
| 571 | DRCILHCAN | L25 | YIDIGNYTVS | L68 | QHMVVKAAL | L28; L35; L34 |
| 572 | DRDAAMQRKL | L35 | YIDIGNYTVSC | L68 | QIAPGQTGK | L6; L7; L19 |
| 573 | DRLITGRL | L25 | YIFFASFYYV | L20 | QKKQQTVTL | L44; L27; L39 |
| 574 | DRLITGRLQ | L25 | YILANTCTER | L17 | QLDEEQPMEI | L5; L2; L68 |
| 575 | DRLITGRLQSL | L25 | YIRVGARK | L43 | QLHNDILLA | L3; L2; L24 |
| 576 | DRLNEVAK | L25 | YITTYPGQGL | L60 | QLPAPRTLL | L57; L5; L22 |
| 577 | DRLNQLES | L25 | YIWLGFIA | L50 | QLPPAYTNSF | L9; L57; L5 |
| 578 | DRLNQLESKM | L30 | YKAFKQIVE | L44 | QLPQGTTL | L57; L23; L5 |
| 579 | DRQTAQAA | L25 | YKAIDGGVTR | L19 | QMAPISAMV | L20; L3; L28 |
| 580 | DRVVFVLW | L25 | YKENSYTTT | L27 | QPGVAMPNL | L22; L56; L39 |
| 58 | DRYFKYWD | L25 | YKENSYTTTI | L34 | QPITNCVKML | L32; L39; L22 |
| 582 | DRYFKYWDQ | L25 | YKHITSKETL | L44 | QPPQTSITSA | L50; L51; L22 |
| 583 | DRYFKYWDQTY | L30 | YKHWPQIAQF | L27 | QPQLEMEL | L39; L22; L56 |
| 584 | DSAEVAVK | L19 | YKKVDGVVQ | L27 | QPRTFLLKY | L31; L11; L22 |
| 585 | DSATLPKGI | L10 | YKLEGYAFE | L44 | QPSVGPKQA | L51; L22; L39 |
| 586 | DSATLVSDI | L10 | YKLMGHFAW | L27 | QPTSEAVEA | L50; L51; L32 |
| 587 | DSCKRVLNV | L73 | YKLNVGDY | L27 | QQLIRAAEI | L48; L25; L24 |
| 588 | DSCNNYMLTY | L18 | YKLNVGDYFVL | L44 | QQQQGQTV | L48; L24; L28 |
| 589 | DSDLNDFV | L1 | YKLPDDFTGC | L27 | QSASKIITLK | L7; L6; L19 |
| 590 | DSDLNDFVS | L1 | YKNTCDGTTF | L27 | QSIIAYTM | L55; L48; L54 |
| 591 | DSEPVLKGV | L20 | YKNTCDGTTFT | L27 | QSINFVRI | L48; L73; L55 |
| | | | Y | | | |
| 592 | DSFKEELDK | L19 | YKSVNITFEL | L44 | QSINFVRIIMR | L19; L21; L7 |
| 593 | DSFVIRGD | L65 | YKYFSGAM | L27 | QSSYIVDSV | L20; L73; L70 |
| 594 | DSFVIRGDEV | L20 | YKYFSGAMD | L27 | QTFFKLVNKF | L11; L10; L52 |
| 595 | DSKEGFFTYI | L10 | YLALYNKYK | L6 | QTLLALHRSY | L52; L14; L40 |
| 596 | DSVEEVLSEAR | L19 | YLASGGQPIT | L2 | QTNSPRRAR | L15; L21; L17 |
| 597 | DSVTVKNGSI | L10 | YLDGADVT | L1 | QVDLFRNAR | L21; L17; L15 |
| 598 | DSYFVVKRH | L19 | YLGKPREQI | L2 | QVRDVLVR | L21; L15; L17 |
| 599 | DTANPKTPKYK | L19 | YLGTGPEAGL | L2 | QVTCGTTTL | L32; L56; L10 |
| 600 | DTDLTKPYI | L1 | YLITPVHVMSK | L6 | QVVSDIDYV | L20; L11; L5 |
| 601 | DTFNGECPNFV | L20 | YLKHGGGVAGA | L3 | QWLPTGTL | L33; L71; L9 |
| 602 | DTKRGVYCC | L10 | YLKLRSDV | L23 | QWNLVIGF | L33; L62; L29 |
| 603 | DTLRVEAF | L29 | YLKLRSDVL | L23 | QYIDIGNYT | L9; L8; L72 |
| 604 | DTPNNTDF | L11 | YLKLRSDVLL | L23 | QYIDIGNYTV | L8; L20; L9 |
| 605 | DTSLSGFKLK | L19 | YLNSTNVTIA | L3 | QYNRYLAL | L71; L72; L66 |
| 606 | DTTEAFEK | L19 | YLNTLTLA | L3 | RAAEIRASA | L51; L50; L46 |
| 607 | DTTITVNVLAW | L10 | YLPIDKCSR | L57 | RAFDIYND | L65; L54; L55 |
| 608 | DTVRTNVYLAVF | L10 | YLPYPDPSRIL | L57 | RAFGEYSHVV | L24; L70; L69 |
| 609 | DTYPSLET | L25 | YLRKHFSM | L23 | RAKVTSAM | L43; L70; L59 |
| 610 | DVDLGDISGI | L10 | YLRKHFSMM | L3 | RALTGIAVE | L52; L44; L53 |
| 611 | DVENPHLMGWD | L1 | YLVQQESPF | L26 | RDAAMQRKL | L33; L55; L37 |
| | Y | | | | | |
| 612 | DVETKDVV | L23 | YLVQQESPFV | L2 | REAACCHL | L33; L37; L36 |
| 613 | DVGDSAEV | L20 | YLYALVYFLQ | L12 | REAACCHLA | L38; L42; L46 |
| 614 | DVGDSAEVAV | L20 | YLYRLFRKS | L24 | REAACCHLAKA | L46; L38; L42 |
| 615 | DVGDSAEVAVK | L19 | YMGTLSYEQF | L9 | REAVGTNLPL | L36; L41; L40 |
| 616 | DVKCTSVV | L23 | YMPASWVMRI | L5 | REEAIRHVRAW | L40; L38; L41 |
| 617 | DVKCTSVVLL | L11 | YMRSLKVPA | L50 | REGYLNSTNV | L45; L38; L36 |
| 618 | DVLECNVKT | L11 | YMRSLKVPATV | L3 | RELGVVHN | L33; L37; L29 |
| 619 | DVLECNVKTT | L10 | YNLWNTFTR | L17 | RELNGGAY | L29; L14; L27 |
| 620 | DVLLPLTQ | L25 | YNYEPLTQ | L48 | RELNGGAYT | L36; L45; L40 |
| 621 | DVLLPLTQYNR | L17 | YNYKLPDDF | L12 | RFASVYAWNR | L15; L21; L17 |
| 622 | DVLVRGFGD | L17 | YNYLYRLFR | L17 | RFLYIIKLIF | L8; L9; L12 |
| 623 | DVQQWGFTGN | L10 | YPANSIVC | L47 | RFYRLANEC | L72; L71; L8 |
| 624 | DVRVVLDF | L23 | YPGQGLNGYTV | L50 | RGFGDSVEE | L65; L44; L27 |
| 625 | DVSFLAHI | L20 | YPLECIKDLL | L31 | RGIVFMCVEY | L26; L43; L27 |
| 626 | DVTQLYLGG | L11 | YPLECIKDLLA | L50 | RHFDEGNCDTL | L34; L28; L16 |
| 627 | DVVECLKLSH | L11 | YPLTKHPNQEY | L31 | RHHANEYRLY | L14; L27; L18 |
| 628 | DVVIGIVNNTV | L20 | YPNMFITREEA | L50 | RHVRAWIGF | L27; L34; L53 |
| 629 | DVVYCPRHV | L20 | YPSLETIQIT | L31 | RIMTWLDMV | L73; L4; L24 |
| 630 | DWTIEYPII | L47 | YPSLETIQITI | L47 | RKGGRTIAF | L27; L44; L33 |
| 631 | DWYDFVENP | L11 | YPTLNISD | L50 | RLANECAQVL | L16; L24; L44 |
| 632 | DYDCVSFC | L62 | YQCAMRPNF | L58 | RLIDAMMF | L26; L16; L54 |
| 633 | DYDCVSFCYM | L62 | YQCGHYKHI | L24 | RLISMMGF | L26; L27; L16 |
| 634 | DYDYYRYNL | L62 | YQLYSTQLS | L24 | RLKLFDRY | L14; L18; L26 |
| 635 | DYFNKKDWYDF | L8 | YQPYRVVV | L48 | RLNQLESKM | L14; L16; L24 |
| 636 | DYGARFYF | L9 | YRAFDIYNDK | L30 | RLQSLENVAF | L16; L26; L56 |
| 637 | DYGARFYFY | L12 | YREAACCHL | L34 | RLYECLYR | L21; L15; L6 |
| 638 | DYIATNGPLKV | L8 | YREGYLNST | L35 | RLYECLYRNR | L21; L15; L6 |
| 639 | DYIINLIIK | L17 | YRINWITGG | L30 | RMYIFFASFYY | L14; L18; L12 |
| 640 | DYILANTC | L71 | YRKVLLRKN | L64 | RNLQHRLY | L55; L14; L65 |
| 641 | DYILANTCTER | L17 | YRLANECAQV | L30 | RNSTPGSSR | L15; L21; L17 |
| 642 | DYKAFKQIV | L23 | YRLANECAQVL | L35 | RNYVFTGY | L27; L26; L65 |
| 643 | DYLVSTQEFR | L17 | YRLFRKSNLK | L30 | RPINPTDQSSY | L14; L27; L31 |
| 644 | DYNYKLPDDF | L9 | YRLYLDAY | L66 | RPLLESEL | L56; L22; L39 |
| 645 | DYVPLKSA | L25 | YRNRDVDTD | L65 | RPNFTIKGSF | L39; L22; L56 |
| 646 | DYYQLYSTQ | L17 | YRRLISMM | L66 | RPNFTIKGSFL | L56; L39; L22 |
| 647 | DYYRSLPGV | L47 | YRVVVLSFELL | L30 | RPPLNRNYV | L39; L22; L51 |
| 648 | DYYRSLPGVF | L9 | YSDSPCES | L1 | RPPLNRNYVF | L22; L39; L56 |
| 649 | EAACCHLAK | L19 | YSDSPCESHG | L63 | RPQIGVVR | L56; L15; L39 |
| 650 | EAALCTFL | L74 | YSDVENPHLMG | L1 | RQALLKTV | L48; L24; L33 |
| | | | W | | | |
| 651 | EAANFCALI | L20 | YSFLPGVYSVIY | L1 | RQKKQQTV | L48; L24; L26 |
| 652 | EAARVVRSIFSR | L19 | YSFVSEET | L43 | RQKKQQTVT | L26; L27; L44 |
| 653 | EAELAKNVSL | L68 | YSLLLCRM | L59 | RQKRTATKAY | L26; L27; L14 |
| 654 | EAEVQIDR | L17 | YSLRLIDAM | L67 | RQRLTKYTM | L27; L44; L26 |
| 655 | EAFLIGCNYL | L20 | YSPIFLIVAA | L50 | RSDVLLPLTQY | L18; L1; L14 |
| 656 | EAGLPYGANK | L19 | YSVIYLYLTFY | L1 | RSGETLGV | L73; L48; L24 |
| 657 | EAIRHVRAWI | L20 | YSVLYNSA | L50 | RSVSPKLFIR | L15; L21; L7 |
| 658 | EAKTVLKKC | L10 | YTDFATSACV | L1 | RTTNGDFL | L55; L54; L53 |
| 659 | EASFNYLK | L19 | YTDFATSACVL | L68 | RTVAGVSI | L55; L54; L53 |
| 660 | EAVGTNLPLQL | L20 | YTEISFMLWC | L1 | RVDFCGKGYHL | L18; L63; L68 |
| 661 | EAVMYMGTLSY | L11 | YTEKWESGV | L1 | RVDGQVDLFR | L21; L7; L18 |
| 662 | EAYEQAVA | L50 | YTEKWESGVK | L1 | RVEAFEYY | L18; L1; L14 |
| 663 | ECAQVLSEMV | L20 | YTELEPPCR | L1 | RVESSSKL | L63; L18; L54 |
| 664 | ECFDKFKVN | L10 | YTELEPPCRF | L1 | RVLNVVCK | L7; L13; L6 |
| 665 | ECFVKRVD | L65 | YTERSEKS | L1 | RVLNVVCKT | L13; L24; L6 |
| 666 | ECNQMCLSTL | L10 | YTGNYQCGHY | L1 | RVLSNLNLP | L6; L16; L15 |
| 667 | ECNVKTTEV | L23 | YTKRNVIPTI | L10 | RVQPTESI | L54; L16; L24 |
| 668 | ECTTIVNGV | L20 | YTKVDGVDVEL | L52 | RVQPTESIV | L24; L14; L28 |
| | | | F | | | |
| 669 | ECTTIVNGVRR | L19 | YTMADLVYA | L20 | RVRQALLK | L13; L6; L7 |
| 670 | ECVRGTTV | L48 | YTPHTVLQA | L11 | RVRQALLKT | L13; L6; L51 |
| 671 | ECVRGTTVLL | L20 | YTSALLAGT | L20 | RVRQALLKTV | L13; L3; L51 |
| 672 | EDDSEPVL | L28 | YTSKTTVASL | L20 | RVYSSANNCTF | L16; L54; L52 |
| 673 | EDDSEPVLK | L19 | YTSNPTTFH | L58 | RWVLNNDYY | L14; L18; L12 |
| 674 | EDIQLLKSA | L46 | YTTTIKPV | L70 | RYWEPEFYE | L65; L8; L9 |
| 675 | EDIQLLKSAY | L40 | YTVELGTEVNEF | L10 | SAEVAVKM | L63; L62; L68 |
| 676 | EDLLFNKVTL | L23 | YTVSCLPFTI | L20 | SAFFGMSR | L19; L17; L15 |
| 677 | EDMLNPNY | L29 | YVDNSSLTIK | L6 | SAFYILPSII | L69; L20; L70 |
| 678 | EDNQTTTI | L48 | YVDNSSLTIKK | L7 | SAGFPFNKW | L49; L52; L54 |
| 679 | EEAALCTFLLN | L41 | YVGCHNKCAY | L43 | SAIGKIQDS | L43; L49; L58 |
| 680 | EEAARYMRS | L42 | YVMHANYIFW | L10 | SARHINAQV | L20; L58; L70 |
| 681 | EEAKKVKPT | L42 | YVPLKSAT | L57 | SAVGNICY | L70; L58; L69 |
| 682 | EEEEFEPST | L42 | YVPLKSATC | L57 | SCKRVLNVV | L51; L3; L39 |
| 683 | EEEGDCEEE | L42 | YVSQPFLMDL | L20 | SDDAVVCF | L33; L62; L63 |
| 684 | EEHFIETIS | L42 | YVTQQLIRAA | L50 | SDRVVFVL | L33; L65; L29 |
| 685 | EELFYSYATH | L40 | YVYIGDPA | L50 | SEAFLIGCNY | L40; L41; L29 |
| 686 | EEMLDNRA | L42 | YVYIGDPAQ | L67 | SEAFLIGCNYL | L40; L36; L41 |
| 687 | EEQEEDWL | L29 | YVYNPFMI | L47 | SEAKCWTET | L46; L42; L37 |
| 688 | EEQPMEID | L29 | YVYNPFMIDV | L20 | SEETGTLI | L45; L36; L37 |
| 689 | EERLKLFDR | L17 | YWDQTYHP | L62 | SEFRVYSS | L29; L33; L37 |
| 690 | EERLKLFDRYF | L40 | YWDQTYHPN | L62 | SEGLNDNL | L33; L36; L37 |
| 691 | EESSAKSASVYY | L41 | YWEPEFYEAMY | L1 | SEISMDNSPNL | L36; L41; L40 |
| 692 | EETGLLMPLKA | L42 | YWFFSNYLK | L7 | SEKQVEQKIA | L42; L38; L46 |
| 693 | EETGTLIVNSVL | L41 | YWFFSNYLKR | L19 | SEKSYELQTP | L42; L46; L38 |
| 694 | EETIYNLLKD | L41 | YYDSMSYE | L62 | SELLTPLGIDL | L36; L41; L40 |
| 695 | EETKFLTENLL | L41 | YYDSMSYED | L62 | SEQLDFIDT | L42; L40; L38 |
| 696 | EETKFLTENLLL | L41 | YYFMRFRR | L17 | SFCYMHHM | L62; L72; L71 |
| 697 | EEVGHTDLMA | L42 | YYFMRFRRA | L46 | SFCYMHHMEL | L71; L72; L62 |
| 698 | EEVKPFIT | L29 | YYGNALDQ | L71 | SFGGASCCL | L72; L71; L62 |
| 699 | EEVLSEAR | L29 | YYGNALDQAI | L9 | SFGGVSVI | L72; L71; L48 |
| 700 | EEVQELYSPIFL | L41 | YYHTTDPS | L71 | SFIEDLLEN | L12; L8; L72 |
| 701 | EEVTTTLEE | L41 | YYLFDESG | L71 | SFIEDLLENK | L7; L13; L6 |
| 702 | EEVVLKTG | L29 | YYLGTGPE | L71 | SFKKGAKL | L72; L62; L71 |
| 703 | EFCSQHTML | L8 | YYLGTGPEAGL | L9 | SFLEMKSEK | L71; L72; L13 |
| 704 | EFDRDAAM | L62 | YYNTTKGGR | L17 | SFNYLKSPNF | L9; L8; L12 |
| 705 | EFDRDAAMQR | L17 | YYRARAGEA | L72 | SFTRGVYY | L72; L71; L12 |
| 706 | EFEPSTQY | L1 | YYRRATRR | L17 | SFTVEKGIY | L14; L12; L72 |
| 707 | EFIERYKL | L8 | YYRSLPGV | L48 | SFVSEETGTL | L8; L72; L71 |
| 708 | EFKLASHMY | L14 | YYSLLMPILTL | L9 | SFYPPDED | L72; L71; L62 |
| 709 | EFLRDGWEIVKF | L8 | AAAYYVGY | L43; L58 | SGVKDCVVL | L44; L32; L56 |
| 710 | EFLTRNPAWR | L17 | AADPAMHA | L63; L68 | SHAAVDAL | L35; L28; L34 |
| 711 | EFQFCNDPF | L72 | AAFATAQEA | L69; L46 | SHEGKTFYV | L34; L35; L28 |
| 712 | EFYAYLRKH | L17 | AAIMQLFFSY | L12; L14 | SHEGKTFYVL | L34; L35; L28 |
| 713 | EGDCEEEEF | L34 | AAISDYDYYRY | L12; L14 | SHFVNLDNL | L34; L28; L35 |
| 714 | EGFFTYICGF | L11 | AAKLMVVI | L70; L69 | SHQSDIEVT | L28; L34; L35 |
| 715 | EGKTFYVL | L23 | AAKLMVVIP | L69; L42 | SHSQLGGLHL | L34; L28; L35 |
| 716 | EGKTFYVLP | L42 | AALALLLLD | L12; L7 | SHSQLGGLHLL | L34; L28; L35 |
| 717 | EGNFYGPFVDR | L17 | AALCTFLLNK | L7; L6 | SHVVAFNTLL | L34; L28; L35 |
| 718 | EGVLTAVV | L48 | AALGVLMSNL | L74; L67 | SIFSRTLET | L43; L6; L3 |
| 719 | EGVVDYGAR | L17 | AALLADKF | L33; L54 | SIINNTVYTK | L7; L19; L6 |
| 720 | EGYAFEHIVY | L27 | AALLADKFPVL | L67; L31 | SIIQFPNTYL | L11; L20; L6 |
| 721 | EGYLNSTNV | L48 | AANFCALILAY | L12; L31 | SIKNFKSVLYY | L12; L14; L6 |
| 722 | EHDYQIGGYTE | L34 | AAYSRYRI | L70; L47 | SKLINIIIW | L44; L27; L52 |
| 723 | EHEIAWYTE | L34 | AAYSRYRIG | L69; L70 | SKPSKRSF | L27; L66; L57 |
| 724 | EHFIETISLA | L28 | ACFVLAAV | L48; L33 | SKRSFIEDL | L44; L27; L66 |
| 725 | EHIVYGDFS | L34 | ADDLNQLTGY | L18; L1 | SKVQIGEYTF | L27; L44; L34 |
| 726 | EHIVYGDFSH | L34 | ADLDDFSKQL | L40; L37 | SLAEYHNESGL | L2; L3; L4 |
| 727 | EHSWNADLY | L34 | ADLVYALRHF | L40; L33 | SLDNVLSTFI | L5; L68; L2 |
| 728 | EHVNNSYECDI | L34 | ADSFVIRGD | L65; L33 | SLFDMSKFPLK | L6; L7; L13 |
| 729 | EHVTFFIY | L29 | ADVEWKFYD | L65; L33 | SLFDMSKFPLKL | L2; L3; L4 |
| 730 | EHYVRITGLY | L11 | AEETRKLMPV | L42; L38 | SLGAENSVAY | L12; L26; L14 |
| 731 | EIDRLNEVA | L42 | AEHVNNSYEC | L42; L38 | SLIDLQELG | L2; L4; L12 |
| 732 | EIIGYKAI | L10 | AEILLIIMRTFK | L40; L41 | SLKELLQNG | L3; L13; L4 |
| 733 | EIIGYKAID | L11 | AEIPKEEVKPF | L41; L40 | SLKVPATV | L3; L23; L43 |
| 734 | EIIGYKAIDGG | L11 | AEIVDTVSALVY | L41; L40 | SLPSYAAF | L57; L43; L72 |
| 735 | EIIKSQDL | L23 | AELAKNVSLD | L40; L41 | SLPVLQVRDV | L57; L64; L5 |
| 736 | EIIKSQDLSV | L11 | AELEGIQYGRSG | L41; L40 | SLREVRTIK | L13; L6; L21 |
| 737 | EIKDATPSD | L11 | AETLKATEE | L45; L46 | SLRVCVDTV | L3; L4; L2 |
| 738 | EIKDTEKYCAL | L10 | AETLKATEETF | L41; L36 | SLSEQLRKQ | L3; L4; L14 |
| 739 | EIKLAKKF | L23 | AEVAVKMFDAY | L40; L41 | SLSHFVNL | L74; L56; L4 |
| 740 | EILGTVSW | L10 | AEVQIDRLIT | L41; L40 | SLVVRCSFY | L14; L12; L26 |
| 741 | EILQKEKV | L23 | AEWFLAYILFT | L40; L41 | SNHNFLVQA | L46; L51; L42 |
| 742 | EIPKEEVKP | L11 | AEYHNESGLKTI | L45; L41 | SNYLKRRV | L48; L45; L33 |
| 743 | EIPVAYRKVL | L57 | AFATAQEA | L72; L71 | SPCESHGKQV | L39; L51; L22 |
| 744 | EISFMLWCK | L19 | AFDKSAFV | L62; L63 | SPFVMMSA | L50; L51; L25 |
| 745 | EITVATSRTL | L10 | AFDKSAFVNL | L62; L68 | SPIFLIVA | L50; L51; L22 |
| 746 | EIVGGQIV | L20 | AFFGMSRIGM | L71; L72 | SPISEHDYQI | L32; L49; L34 |
| 747 | EKCSAYTVEL | L44 | AFKLNIKL | L62; L71 | SPNLAWPL | L22; L56; L39 |
| 748 | EKDEDDNLI | L34 | AFLIGCNY | L71; L72 | SPSGVYQCAM | L22; L32; L39 |
| 749 | EKGDYGDAVVY | L27 | AFPFTIYS | L71; L72 | SQCVNLTTR | L15; L21; L17 |
| 750 | EKQEILGTV | L20 | AFPSGKVE | L71; L72 | SQDLSVVSKV | L63; L68; L24 |
| 751 | ELAKNVSLD | L11 | AFPSGKVEG | L72; L71 | SQILPDPSK | L13; L7; L28 |
| 752 | ELDSFKEELDKY | L1 | AFQTVKPG | L72; L71 | SQLGGLHLLI | L24; L34; L8 |
| 753 | ELFYSYATH | L10 | AFVETVKGLDY | L12; L71 | SQNAVASKI | L24; L48; L34 |
| 754 | ELGKYEQYI | L24 | AFVNLKQL | L72; L71 | SQWLTNIF | L27; L48; L26 |
| 755 | ELGTDPYEDF | L11 | AFVTNVNAS | L72; L71 | SRELKVTFF | L34; L30; L62 |
| 756 | ELHLSWEV | L23 | AGFPFNKW | L54; L48 | SRIGMEVTP | L35; L34; L30 |
| 757 | ELIRQGTDYKHW | L10 | AGFPFNKWGK | L7; L13 | SRIIPARAR | L30; L17; L15 |
| 758 | ELKHGTFTC | L10 | AGGIVAIVV | L69; L73 | SRNYIAQV | L64; L35; L48 |
| 759 | ELKINAAC | L23 | AGLIAIVMV | L4; L73 | SRNYIAQVD | L65; L64; L35 |
| 760 | ELKINAACR | L17 | AGNVQLRV | L48; L73 | SRVPDLLV | L64; L35; L25 |
| 761 | ELKKLLEQ | L23 | AGNVQLRVI | L55; L48 | SRYRIGNYKL | L30; L35; L34 |
| 762 | ELSLIDFY | L1 | AGSDKGVAP | L46; L42 | SRYWEPEF | L65; L66; L28 |
| 763 | ELSLIDFYL | L20 | AGTDLEGNFY | L14; L12 | SSRGTSPAR | L21; L15; L13 |
| 764 | ELSPVALRQM | L11 | AHIQWMVM | L28; L35 | SSRSRNSSR | L15; L21; L17 |
| 765 | ELSRVLGL | L23 | AHISTIGV | L28; L35 | SSSDNIALLV | L73; L20; L18 |
| 766 | ELTGHMLDM | L11 | AHKDKSAQCF | L43; L34 | SSVELKHFFF | L52; L53; L54 |
| 767 | ELTPVVQTIEV | L20 | AHVASCDAI | L34; L28 | STASALGK | L7; L19; L6 |
| 768 | ELTSMKYFV | L20 | AIAMACLVGL | L3; L4 | STASDTYACW | L10; L52; L54 |
| 769 | ELVAELEGIQY | L11 | AIGKIQDSL | L67; L44 | STDGNKIADKY | L18; L1; L14 |
| 770 | ELVIGAVI | L23 | AINRPQIGVVR | L21; L15 | STFNVPMEKLK | L7; L6; L13 |
| 771 | EMELTPVV | L48 | AIPTNFTISV | L57; L5 | STKPVETSNSF | L52; L53; L10 |
| 772 | EMIAQYTSA | L42 | AITILDGISQY | L14; L18 | STMTNRQF | L52; L55; L53 |
| 773 | EMIAQYTSAL | L10 | AKDTTEAF | L27; L68 | STQLGIEFLKR | L15; L7; L19 |
| 774 | EMKSEKQV | L48 | AKHDFFKF | L27; L66 | STYASQGLV | L20; L73; L14 |
| 775 | EMLAKALR | L17 | AKHDFFKFR | L15; L21 | SVEEVLSEA | L3; L5; L51 |
| 776 | EMLAKALRK | L6 | AKKVKPTVV | L23; L3 | SVITPGTNT | L13; L11; L7 |
| 777 | EMVMCGGSL | L10 | AKNLNESL | L44; L27 | SVMLTNDNTSR | L7; L19; L15 |
| 778 | EMYLKLRSD | L23 | AKNRARTVA | L42; L46 | SVQTFFKLVNK | L7; L6; L15 |
| 779 | ENFNQHEVLL | L20 | AKSHNIALI | L44; L34 | SVVNARLRAK | L7; L10; L6 |
| 780 | ENSYTTTI | L48 | ALALLLLDRL | L4; L3 | SVVNIQKEI | L20; L10; L24 |
| 781 | ENVAFNVVNK | L19 | ALAPNMMVT | L3; L2 | SVVSKVVK | L7; L19; L13 |
| 782 | EPEFYEAMY | L31 | ALDPLSETKC | L5; L18 | SYCTGYREGY | L14; L72; L71 |
| 783 | EPIYDEPTT | L32 | ALDQAISMWAL | L5; L68 | SYELQTPF | L71; L72; L62 |
| 784 | EPKKDKKKK | L23 | ALEPLVDLP | L1; L18 | SYFVVKRH | L71; L48; L72 |
| 785 | EPKKDKKKKAD | L23 | ALFAYTKR | L21; L15 | SYGIATVREV | L72; L64; L71 |
| 786 | EPLTQDHV | L47 | ALFAYTKRN | L24; L3 | SYKDWSYSG | L8; L66; L15 |
| 787 | EPLTQDHVD | L32 | ALFAYTKRNV | L3; L2 | TAALGVLM | L70; L31; L74 |
| 788 | EPLVDLPI | L47 | ALGGSVAIK | L6; L7 | TADIVVFD | L68; L63; L62 |
| 789 | EPLVDLPIG | L31 | ALGKLQDVV | L24; L2 | TADIVVFDEI | L67; L68; L49 |
| 790 | EPMLQSADA | L50 | ALIISVTSN | L3; L26 | TANVNALL | L62; L68; L63 |
| 791 | EPQIITTD | L23 | ALITLATCEL | L2; L4 | TAYNGYLTS | L49; L43; L69 |
| 792 | EPTTTTSVPL | L22 | ALKGGKIV | L23; L39 | TCCSLSHRF | L49; L8; L34 |
| 793 | EQDKNTQEVF | L34 | ALKGGKIVN | L43; L26 | TDDNALAYY | L1; L18; L14 |
| 794 | EQIDGYVM | L35 | ALKGGKIVNN | L3; L21 | TDNYITTY | L29; L65; L33 |
| 795 | EQIDGYVMHA | L11 | ALLADKFPV | L2; L4 | TEAFEKMV | L45; L37; L38 |
| 796 | EQKSILSPL | L10 | ALLAGTITS | L2; L4 | TEAFEKMVSLL | L36; L40; L41 |
| 797 | EQKSILSPLY | L14 | ALLLLDRL | L4; L74 | TECSNLLL | L45; L36; L37 |
| 798 | EQLDFIDTKR | L17 | ALLSDLQDLK | L6; L7 | TEGSVKGL | L37; L33; L45 |
| 799 | EQPTSEAV | L48 | ALLSTDGNK | L6; L7 | TEGSVKGLQP | L38; L42; L46 |
| 800 | EQYIKWPW | L48 | ALLSTDGNKI | L24; L2 | TEIYQAGSTP | L38; L46; L42 |
| 801 | EQYIKWPWYI | L48 | ALLTKSSEYK | L6; L7 | TEKYCALAP | L46; L42; L38 |
| 802 | EQYVFCTVN | L48 | ALLTLQQI | L4; L24 | TELEPPCRFV | L45; L38; L36 |
| 803 | ERLKLFAA | L25 | ALNDFSNSG | L43; L16 | TENLLLYIDI | L41; L40; L36 |
| 804 | ERLKLFDR | L17 | ALPETTAD | L57; L43 | TESNKKFLP | L38; L42; L46 |
| 805 | ERLKLFDRYF | L30 | ALPETTADIV | L5; L2 | TETICAPLTV | L45; L38; L46 |
| 806 | ERSEAGVC | L35 | ALPETTADIVV | L5; L2 | TETICAPLTVF | L40; L41; L29 |
| 807 | ERVRQALL | L35 | ALQDAYYR | L21; L15 | TFEEAALCTF | L62; L8; L9 |
| 808 | ERVRQALLK | L30 | ALQDAYYRAR | L21; L15 | TFGAGAAL | L72; L71; L62 |
| 809 | ERYKLEGY | L25 | ALRANSAVK | L13; L6 | TFISDEVAR | L17; L19; L15 |
| 810 | ESCGNFKVTK | L19 | ALRKVPTDNY | L26; L14 | TFLLNKEMY | L12; L72; L71 |
| 811 | ESDDYIAT | L1 | ALSKGVHF | L26; L74 | TFTYASAL | L72; L71; L62 |
| 812 | ESDDYIATN | L1 | ALTCFSTQF | L26; L27 | TFVTHSKGL | L71; L8; L72 |
| 813 | ESFGGASCC | L19 | ALTNNVAF | L26; L43 | TGSNVFQTR | L19; L17; L15 |
| 814 | ESGLKTIL | L23 | ALYYPSAR | L21; L6 | TGYKKPASR | L17; L19; L15 |
| 815 | ESGLKTILRK | L19 | AMACLVGL | L56; L3 | THDVSSAIN | L28; L35; L34 |
| 816 | ESLRPDTR | L17 | AMMFTSDL | L71; L72 | THHWLLLTIL | L28; L34; L35 |
| 817 | ESLVPGFNE | L17 | AMPNMLRIM | L64; L57 | THSDKFTDGVC | L34; L35; L28 |
| | | | | | L | |
| 818 | ESPFELEDF | L11 | ANGDSEVVLK | L7; L6 | THTGTGQAI | L35; L34; L28 |
| 819 | ESSAKSASV | L20 | ANGQVFGL | L65; L33 | THTGTGQAITV | L28; L34; L35 |
| 820 | ESSAKSASVY | L14 | ANGQVFGLYK | L7; L6 | THVQLSLPVL | L28; L34; L35 |
| 821 | ESVQTFFKLV | L20 | ANKDGIIWVA | L51; L42 | TIDGSSGVV | L68; L63; L28 |
| 822 | ETAHSCNV | L20 | ANSTVLSF | L33; L65 | TIFKDASGK | L19; L6; L7 |
| 823 | ETAQNSVRVLQK | L19 | ANYAKPFLNK | L6; L7 | TISLAGSY | L14; L18; L1 |
| 824 | ETDLTKGPH | L1 | ANYQKVGMQK | L6; L7 | TISLAGSYK | L6; L19; L7 |
| 825 | ETDLTKGPHEF | L1 | APFLYLYA | L50; L51 | TITQMNLKY | L12; L18; L14 |
| 826 | ETFKLSYGIA | L11 | APGQTGKIA | L51; L39 | TITVNVLAW | L10; L16; L49 |
| 827 | ETFKLSYGIATV | L20 | APGTAVLRQWL | L56; L22 | TKDVVECL | L62; L68; L63 |
| 828 | ETFVTHSKGLY | L11 | APHGHVMVE | L51; L39 | TKHPNQEY | L27; L66; L43 |
| 829 | ETFYPKLQS | L20 | APKEIIFLE | L39; L56 | TKPYIKWDL | L44; L66; L57 |
| 830 | ETGLLMPLK | L19 | APLLSAGIF | L22; L31 | TKVDGVDVEL | L44; L32; L66 |
| 831 | ETGTLIVNSV | L20 | APNMMVTNNTF | L39; L22 | TLADAGFIK | L6; L7; L19 |
| 832 | ETICAPLTVFF | L11 | APRTLLTK | L22; L51 | TLADAGFIKQY | L11; L10; L14 |
| 833 | ETIQITISSFK | L19 | APRTLLTKG | L51; L22 | TLEPEYFNSV | L5; L2; L4 |
| 834 | ETISLAGS | L11 | APSASAFF | L22; L56 | TLIVNSVL | L23; L68; L28 |
| 835 | ETIYNLLK | L19 | APSASAFFGM | L22; L56 | TLKATEETF | L26; L9; L16 |
| 836 | ETKDVVEC | L17 | APYIVGDV | L47; L51 | TLKKRWQLAL | L23; L3; L39 |
| 837 | ETKDVVECLK | L19 | APYIVGDVVQ | L56; L51 | TLLSLREVR | L15; L21; L17 |
| 838 | ETKFLTENLL | L20 | AQAAGTDTTI | L34; L24 | TLNGLWLDDV | L2; L4; L3 |
| 839 | ETKFLTENLLLY | L1 | AQALNTLV | L24; L48 | TLQAENVTGL | L4; L3; L2 |
| 840 | ETLGVLVPHV | L20 | AQCFKMFYK | L7; L6 | TLSYEQFKK | L6; L7; L19 |
| 841 | ETLPTEVLT | L17 | AQDGNAAISDY | L14; L18 | TLVFLFVAA | L50; L46; L51 |
| 842 | ETLYCIDGALL | L20 | AQEAYEQAV | L24; L28 | TMCDIRQLLF | L12; L8; L9 |
| 843 | ETNILLNVPL | L20 | AQNSVRVLQ | L44; L15 | TNNLVVMAY | L14; L31; L65 |
| 844 | ETQALPQRQ | L20 | AQSFLNRV | L48; L24 | TPLVPFWIT | L31; L47; L50 |
| 845 | ETQALPQRQK | L19 | AQTGIAVLD | L44; L24 | TPLVPFWITI | L47; L50; L22 |
| 846 | ETRKLMPVCV | L20 | AQVAKSHNI | L24; L48 | TPTWRVYST | L23; L32; L39 |
| 847 | ETSNSFDV | L20 | AQVDVVNEN | L24; L44 | TQLGIEFL | L25; L35; L34 |
| 848 | ETSNSFDVLK | L19 | AQVKQIYKT | L24; L44 | TQLGIEFLK | L7; L30; L6 |
| 849 | ETSWQTGDFV | L20 | AQYTSALLA | L24; L28 | TQVVDMSMTY | L26; L27; L14 |
| 850 | ETTADIVV | L20 | ARAGKASCTL | L30; L35 | TQWSLFFFLY | L14; L12; L27 |
| 851 | ETTADIVVFD | L20 | ARDGCVPLNI | L30; L34 | TRAKVGILC | L30; L34; L35 |
| 852 | ETYFTQSRN | L11 | ARHINAQV | L35; L25 | TREEAIRHV | L64; L35; L34 |
| 853 | EVARDLSL | L20 | ARLRAKHYVY | L66; L30 | TRLQSLENV | L30; L34; L35 |
| 854 | EVAVKMFDAYV | L20 | ARNGVLIT | L65; L35 | TRQVVNVV | L35; L25; L64 |
| 855 | EVFAQVKQ | L20 | ARNGVLITEG | L65; L30 | TRVECTTI | L25; L35; L34 |
| 856 | EVFNATRFAS | L20 | ASAVVLLILM | L52; L7 | TRVECTTIV | L64; L35; L25 |
| 857 | EVFNATRFASV | L20 | ASDTYACWH | L18; L1 | TSAFVETV | L48; L20; L70 |
| 858 | EVGKPRPPL | L20 | ASEAARVVR | L15; L21 | TSAMQTMLF | L53; L52; L54 |
| 859 | EVGKPRPPLNR | L17 | ASEFSSLP | L1; L18 | TSDLATNNLV | L1; L68; L18 |
| 860 | EVGPEHSLA | L20 | ASEYTGNY | L1; L18 | TSFGPLVRKI | L55; L20; L10 |
| 861 | EVITFDNLKT | L11 | ASEYTGNYQ | L1; L18 | TSLEIPRR | L48; L17; L15 |
| 862 | EVKILNNL | L23 | ASFYYVWK | L7; L13 | TSNSFDVL | L73; L55; L67 |
| 863 | EVKILNNLGV | L11 | ASGKPVPY | L43; L65 | TSPISEHDY | L14; L1; L26 |
| 864 | EVKPFITE | L23 | ASGNLLLDK | L7; L6 | TSSGDATTAY | L18; L14; L1 |
| 865 | EVLLAPLL | L74 | ASKKPRQK | L13; L55 | TSTDVVYR | L19; L17; L15 |
| 866 | EVLLAPLLSA | L50 | ASLPFGWLIV | L73; L69 | TSVDCTMYI | L70; L69; L73 |
| 867 | EVLSDRELH | L17 | ASLVLARKH | L55; L52 | TSVVLLSV | L48; L73; L59 |
| 868 | EVLSDRELHL | L10 | ASSSEAFL | L74; L73 | TTCCSLSHR | L17; L15; L19 |
| 869 | EVNEFACV | L20 | ASTDTCFANK | L7; L13 | TTCFSVAAL | L20; L68; L57 |
| 870 | EVPANSTV | L20 | ATALLTLQQ | L7; L18 | TTFTYASALW | L52; L10; L54 |
| 871 | EVPVSIINNTV | L20 | ATCDWTNAGDY | L14; L18 | TTIAKNTVK | L7; L19; L6 |
| 872 | EVQELYSPIF | L11 | ATCFSTASDTY | L18; L14 | TTIKPVTYKL | L20; L53; L11 |
| 873 | EVQELYSPIFL | L20 | ATEETFKLS | L1; L18 | TTLDSKTQSL | L10; L16; L8 |
| 874 | EVQGYKSVNI | L20 | ATEGALNTP | L42; L18 | TTLEETKF | L53; L54; L52 |
| 875 | EVQPQLEMEL | L20 | ATLVSDIDITF | L52; L53 | TTLNGLWL | L55; L61; L60 |
| 876 | EVRQIAPGQ | L11 | ATNNAMQV | L73; L70 | TTTLEETKF | L53; L52; L54 |
| 877 | EVRTIKVFT | L20 | ATNNLVVMA | L42; L69 | TTVDNINL | L20; L70; L60 |
| 878 | EVRTIKVFTTV | L20 | ATNVVIKVCEF | L52; L16 | TTYKLNVGDY | L14; L11; L10 |
| 879 | EVTGDSCNNYM | L11 | ATTAYANSV | L73; L70 | TVCTVCGMWK | L7; L6; L19 |
| 880 | EVTTTLEET | L10 | ATVAYFNMV | L73; L20 | TVEEAKTVLK | L19; L7; L6 |
| 881 | EVVDKYFD | L20 | ATVAYFNMVY | L14; L18 | TVELGTEV | L63; L68; L23 |
| 882 | EVVENPTI | L20 | ATVREVLSD | L65; L10 | TVFFDGRVD | L65; L55; L32 |
| 883 | EWKFYDAQP | L42 | AVAKHDFF | L74; L54 | TVMPLSAPTL | L20; L28; L32 |
| 884 | EYCRHGTCER | L17 | AVAKHDFFK | L7; L6 | TVNVLAWL | L62; L59; L23 |
| 885 | EYDYVIFT | L62 | AVANGDSEVVL | L44; L56 | TVQFCDAMR | L19; L21; L17 |
| 886 | EYDYVIFTQ | L17 | AVANGDSEVVL | L7; L6 | TVYDDGARRV | L16; L52; L10 |
| | | | K | | W | |
| 887 | EYFNSVCR | L17 | AVCRHHANEY | L18; L14 | TVYDPLQPE | L6; L43; L20 |
| 888 | EYRLYLDAY | L14 | AVDAAKAYK | L7; L6 | TVYSHLLLVA | L51; L3; L46 |
| 889 | EYTDFATSA | L17 | AVINGDRWFLN | L7; L15 | TVYTKVDGV | L3; L20; L11 |
| | | | R | | | |
| 890 | EYTFEKGDY | L14 | AVKMFDAY | L14; L26 | TWFSQRGGSY | L14; L10; L12 |
| 891 | EYVSQPFL | L9 | AVKTQFNYYK | L13; L7 | TWICLLQF | L8; L9; L59 |
| 892 | FAAYSRYR | L17 | AVKTQFNYYKK | L7; L13 | TYASALWEI | L9; L8; L64 |
| 893 | FAFACPDGVKH | L31 | AVLQSGFRK | L7; L6 | TYEGNSPF | L72; L71; L62 |
| 894 | FAKFLKTN | L70 | AVRDPQTLE | L13; L6 | TYICGFIQQ | L8; L17; L9 |
| 895 | FAKFLKTNC | L58 | AVSKGFFKE | L7; L24 | TYKLNVGDY | L14; L72; L71 |
| 896 | FAMMFVKH | L59 | AVVIPTKK | L7; L6 | TYLDGADVTK | L8; L9; L17 |
| 897 | FANKHADFD | L58 | AVVIPTKKA | L51; L46 | TYLEGSVRVV | L8; L72; L71 |
| 898 | FANKHADFDTW | L49 | AVVYRGTTTYK | L7; L6 | TYNKVENM | L71; L72; L62 |
| 899 | FASEAARVVR | L19 | AVYRINWI | L48; L24 | TYNLWNTFTR | L17; L15; L21 |
| 900 | FASFYYVWKSY | L31 | AWQPGVAMP | L42; L38 | TYRRLISM | L72; L71; L66 |
| 901 | FATSACVLAA | L50 | AYANRNRFLY | L12; L14 | VAALTNNVAF | L31; L52; L58 |
| 902 | FAVDAAKA | L50 | AYANRNRFLYI | L9; L8 | VAFNVVNKG | L69; L70; L47 |
| 903 | FAYANRNR | L17 | AYCNKTVGEL | L72; L71 | VAITRAKV | L70; L47; L48 |
| 904 | FAYANRNRFLY | L12 | AYIICIST | L71; L72 | VAKYTQLCQY | L43; L14; L58 |
| 905 | FAYANRNRFLYI | L47 | AYIICISTKHFY | L14; L40 | VANGDSEV | L70; L58; L63 |
| 906 | FAYTKRNVIPTI | L47 | AYITGGVV | L72; L71 | VAYESLRPD | L43; L49; L59 |
| 907 | FCAFAVDAA | L50 | AYKIEELFYSY | L14; L12 | VAYSNNSI | L47; L70; L68 |
| 908 | FCGKGYHL | L74 | AYNGYLTS | L65; L71 | VCTEIDPKL | L8; L9; L5 |
| 909 | FCLLNRYFR | L17 | AYNMMISA | L71; L72 | VDDPCPIHFY | L18; L1; L14 |
| 910 | FCSQHTML | L74 | AYNMMISAG | L72; L71 | VDILGPLSA | L46; L50; L51 |
| 911 | FDEDDSEPVL | L67 | AYNVTQAFGR | L15; L21 | VDYGARFY | L65; L14; L55 |
| 912 | FDEGNCDTL | L67 | AYPLTKHP | L72; L71 | VEGCMVQV | L45; L37; L33 |
| 913 | FDEISMATNY | L1 | AYPLTKHPN | L72; L71 | VEHVTFFI | L45; L33; L48 |
| 914 | FDKSAFVNL | L58 | AYRFNGIGV | L13; L72 | VEHVTFFIY | L29; L40; L41 |
| 915 | FDRDAAMQR | L17 | AYRKVLLR | L15; L17 | VEKKKLDGF | L33; L37; L29 |
| 916 | FDTRVLSNL | L74 | AYTRYVDNNF | L9; L8 | VELKHFFFA | L46; L42; L38 |
| 917 | FDVEGCHATR | L19 | AYVDNSSLT | L8; L9 | VEQDKNTQEV | L38; L45; L37 |
| 918 | FEEAALCTFL | L36 | AYWVPRAS | L71; L72 | VEQKIAEIP | L38; L42; L29 |
| 919 | FEHIVYGD | L33 | AYYFMRFRRA | L46; L42 | VESSSKLWA | L46; L38; L42 |
| 920 | FEKGDYGDA | L46 | AYYFMRFRRAF | L71; L72 | VETSNSFDV | L45; L38; L46 |
| 921 | FEKGDYGDAV | L45 | AYYNTTKGGRF | L8; L9 | VEWKFYDAQP | L46; L42; L38 |
| 922 | FELEDFIP | L38 | AYYRARAG | L71; L72 | VFAQVKQIY | L12; L14; L72 |
| 923 | FELLHAPA | L50 | AYYVGYLQPR | L15; L21 | VFCGVDAV | L71; L62; L72 |
| 924 | FELLHAPAT | L38 | CANGQVFGLY | L14; L12 | VFCTVNAL | L72; L71; L62 |
| 925 | FELTSMKY | L29 | CASLKELL | L23; L74 | VFDEISMATNY | L12; L18; L62 |
| 926 | FELTSMKYFV | L45 | CATVHTANKW | L54; L49 | VFHLYLQY | L71; L72; L12 |
| 927 | FELWAKRN | L29 | CAYWVPRA | L50; L47 | VFKNIDGY | L14; L71; L72 |
| 928 | FENKTTLP | L38 | CAYWVPRAS | L59; L69 | VFLFVAAI | L71; L72; L48 |
| 929 | FFASFYYV | L62 | CCDDDYFNK | L13; L7 | VFLLVTLAIL | L9; L62; L8 |
| 930 | FFDGRVDG | L62 | CCYDHVIST | L69; L43 | VFPLNSII | L62; L71; L72 |
| 931 | FFDGRVDGQ | L62 | CDNIKFAD | L65; L33 | VFPPTSFG | L62; L72; L71 |
| 932 | FFDGRVDGQV | L62 | CDVTDVTQL | L37; L33 | VFQSASKIITL | L72; L71; L8 |
| 933 | FFIYNKIV | L71 | CEEEEFEP | L38; L42 | VFQTRAGCL | L72; L71; L62 |
| 934 | FFSNYLKRR | L17 | CEEMLDNRA | L38; L42 | VFTTVDNINL | L9; L8; L72 |
| 935 | FFTLLLQLC | L12 | CEFQFCNDP | L46; L42 | VGSDNVTDF | L26; L9; L8 |
| 936 | FFYVLGLA | L62 | CEIVGGQI | L45; L48 | VHFVCNLL | L28; L34; L35 |
| 937 | FFYVLGLAAIM | L71 | CFSTQFAF | L72; L71 | VHFVCNLLL | L34; L28; L35 |
| 938 | FGADPIHSLR | L19 | CFTNVYADSF | L9; L72 | VHNQDVNL | L28; L34; L35 |
| 939 | FGDDTVIEVQ | L68 | CFVLAAVY | L72; L71 | VIAWNSNNL | L60; L74; L58 |
| 940 | FGDFIQTT | L68 | CFVLAAVYR | L17; L15 | VILRGHLRI | L24; L8; L48 |
| 941 | FGDFIQTTP | L68 | CGFIQQKL | L48; L25 | VINGDRWFLNR | L15; L21; L17 |
| 942 | FGGCVFSY | L66 | CGPDGYPL | L57; L66 | VIVFDGKSK | L7; L13; L43 |
| 943 | FGLFCLLNR | L17 | CHDGKAHF | L62; L63 | VKDCVVLHSY | L14; L18; L27 |
| 944 | FGLFCLLNRY | L12 | CIDCSARHI | L68; L63 | VKQGDDYVY | L27; L66; L14 |
| 945 | FGLVAEWF | L59 | CIKDLLAR | L17; L15 | VKRVDWTIEY | L43; L27; L26 |
| 946 | FGPLVRKIF | L57 | CLAYYFMRF | L12; L10 | VLAAECTIF | L26; L9; L74 |
| 947 | FGWLIVGVA | L50 | CLDDRCILH | L5; L18 | VLHSTQDLF | L9; L12; L26 |
| 948 | FHAIHVSGT | L28 | CLDDRCILHC | L18; L5 | VLHSYFTSDY | L43; L26; L14 |
| 949 | FHLVDFQVT | L35 | CLEASFNY | L1; L18 | VLITEGSVK | L6; L13; L7 |
| 950 | FHLVDFQVTI | L34 | CLEASFNYL | L5; L1 | VLKKCKSAF | L23; L43; L26 |
| 951 | FHQKLLKSI | L34 | CLLQFAYANR | L21; L15 | VLLFLAFVV | L4; L2; L24 |
| 952 | FHTPAFDKSAF | L28 | CLTPVYSF | L23; L26 | VLLILMTAR | L21; L15; L6 |
| 953 | FIASFRLFA | L69 | CMMCYKRNR | L15; L17 | VLLSVLQQLR | L21; L15; L6 |
| 954 | FIDTKRGVYC | L63 | CNIVNVSLV | L73; L48 | VLNNDYYRSL | L43; L57; L4 |
| 955 | FIEDLLEN | L1 | CNLLLLFV | L73; L48 | VLPNDDTL | L57; L5; L62 |
| 956 | FIETISLAGSY | L1 | CPAEIVDTVSA | L50; L51 | VLPNDDTLR | L21; L15; L5 |
| 957 | FISDEVARD | L3 | CPDGVKHVYQL | L32; L39 | VLPNDDTLRV | L5; L2; L57 |
| 958 | FISDEVARDL | L60 | CPFGEVENA | L50; L51 | VLPPLLTD | L57; L5; L43 |
| 959 | FISNSWLM | L58 | CPIFFITGNTL | L56; L22 | VLPQLEQPY | L12; L26; L57 |
| 960 | FISPYNSQN | L58 | CPIHFYSKW | L49; L10 | VLQAVGACV | L2; L5; L4 |
| 961 | FIYNKIVDE | L69 | CPLIAAVI | L47; L48 | VLQKAAITI | L4; L24; L2 |
| 962 | FKDQVILLN | L1 | CRFDTRVLS | L65; L30 | VLQVRDVLV | L2; L4; L5 |
| 963 | FKEELDKY | L1 | CRSKNPLL | L35; L66 | VLSFELLHA | L3; L2; L4 |
| 964 | FKEELDKYF | L27 | CSDKAYKI | L55; L63 | VLSTFISAA | L3; L2; L51 |
| 965 | FKELLVYAA | L50 | CSVIDLLL | L73; L74 | VLYQPPQTS | L43; L3; L24 |
| 966 | FKFVCDNIKF | L27 | CTCGKQATK | L7; L19 | VMCGGSLY | L18; L1; L14 |
| 967 | FKHLIPLM | L66 | CTEIDPKLDNY | L1; L18 | VMFLARGI | L48; L55; L47 |
| 968 | FKLASHMY | L27 | CTEIDPKLDNYY | L1; L18 | VPFWITIA | L50; L51; L47 |
| 969 | FKLNEEIAI | L44 | CTFEYVSQP | L11; L10 | VPNQPYPNA | L51; L50; L22 |
| 970 | FKLSYGIAT | L44 | CTFLLNKEM | L70; L69 | VPQADVEW | L56; L49; L39 |
| 971 | FKVNSTLEQY | L27 | CTVCGMWKGY | L10; L11 | VPVSIINNTV | L51; L50; L47 |
| 972 | FKVSIWNLDY | L12 | CVFSYVGCHNK | L7; L6 | VQIDRLITG | L26; L69; L27 |
| 973 | FKWDLTAFGL | L44 | CVLAAECTIFK | L7; L6 | VQLHNDILL | L44; L8; L34 |
| 974 | FKYWDQTYH | L27 | CVLSGHNLAK | L6; L7 | VQQESPFVM | L66; L35; L28 |
| 975 | FLAHIQWM | L74 | CVVADAVIK | L7; L19 | VQQLPETY | L26; L27; L48 |
| 976 | FLALITLA | L3 | CYFGLFCLLNR | L15; L17 | VQQLPETYF | L26; L27; L9 |
| 977 | FLALITLATC | L3 | CYKRNRATR | L17; L15 | VRAWIGFDV | L64; L30; L35 |
| 978 | FLAYILFTRFFY | L12 | CYNGSPSGV | L62; L72 | VRCSFYEDF | L66; L30; L34 |
| 979 | FLEGETLPTEV | L2 | CYNGSPSGVY | L14; L72 | VRETMSYLF | L9; L66; L34 |
| 980 | FLGIITTVAA | L2 | DAAMQRKL | L47; L70 | VRIKIVQM | L66; L35; L25 |
| 981 | FLGYFCTCY | L12 | DALFAYTKR | L17; L19 | VRTNVYLAV | L35; L64; L30 |
| 982 | FLIGCNYLG | L2 | DALNNIINN | L47; L17 | VSCLPFTI | L55; L48; L68 |
| 983 | FLIGCNYLGK | L6 | DALNNIINNAR | L17; L19 | VSDIDITFLK | L1; L18; L7 |
| 984 | FLKKDAPY | L43 | DAMMFTSDL | L25; L61 | VSDIDITFLKK | L18; L1; L7 |
| 985 | FLKTNCCRF | L43 | DANYFLCW | L49; L47 | VSDVGDSAE | L68; L1; L63 |
| 986 | FLLKYNENG | L2 | DAPYIVGDV | L47; L20 | VSGTNGTKR | L15; L19; L21 |
| 987 | FLLNKEMY | L12 | DAQSFLNR | L17; L19 | VSNGTHWFV | L73; L58; L70 |
| 988 | FLLNKEMYLK | L6 | DATPSDFVR | L19; L17 | VSTSGRWV | L48; L70; L55 |
| 989 | FLLPSLATVA | L2 | DAYPLTKHP | L47; L49 | VSWNLREML | L53; L16; L67 |
| 990 | FLLVTLAI | L59 | DDFTGCVIAW | L10; L29 | VTDFNAIAT | L68; L1; L18 |
| 991 | FLLVTLAILTA | L2 | DDPCPIHFY | L11; L29 | VTDTPKGPKV | L63; L1; L18 |
| 992 | FLMDLEGKQ | L2 | DDYQGKPLEF | L10; L49 | VTHSKGLY | L1; L14; L18 |
| 993 | FLMDLEGKQG | L2 | DEFTPFDV | L29; L25 | VTLLPAADL | L60; L61; L53 |
| 994 | FLNKVVST | L23 | DEFTPFDVVR | L29; L19 | VTMPLGYVT | L52; L55; L53 |
| 995 | FLNRVCGV | L3 | DEFTPFDVVRQ | L41; L29 | VTMPLGYVTH | L52; L53; L7 |
| 996 | FLPFAMGI | L57 | DELTGHMLD | L29; L25 | VTQLYLGGMSY | L1; L14; L18 |
| 997 | FLPFFSNVT | L57 | DEMIAQYTSA | L38; L42 | VTRDIASTD | L43; L55; L13 |
| 998 | FLPFFSNVTWF | L57 | DESGEFKL | L29; L37 | VTTEILPV | L73; L48; L70 |
| 999 | FLPGVYSVIYLY | L12 | DEVRQIAP | L29; L46 | VTVYSHLL | L60; L55; L70 |
| 1000 | FLRDGWEIVK | L13 | DEWSMATYYLF | L41; L40 | VVDADSKIV | L63; L5; L68 |
| 1001 | FLRDGWEIVKF | L43 | DFATSACVL | L72; L71 | VVDADSKIVQL | L68; L5; L63 |
| 1002 | FLVFLGIITT | L2 | DFLELAMDEF | L9; L8 | VVDDPCPI | L68; L63; L62 |
| 1003 | FLVLIMLI | L74 | DFLHFLPRV | L47; L4 | VVDDPCPIH | L63; L68; L5 |
| 1004 | FLWLLWPV | L2 | DFMSLSEQLR | L17; L19 | VVDYGARFYF | L1; L63; L12 |
| 1005 | FLWLLWPVT | L2 | DFTGCVIAW | L49; L10 | VVFDEISM | L69; L59; L58 |
| 1006 | FLYIIKLIFLW | L12 | DFVENPDIL | L62; L67 | VVFLLVTLA | L50; L51; L3 |
| 1007 | FMRFRRAF | L59 | DFVENPDILR | L17; L19 | VVFNGVSFSTF | L16; L26; L52 |
| 1008 | FMRIFTIGTV | L3 | DFVRATATI | L10; L8 | VVIPDYNTYK | L7; L6; L13 |
| 1009 | FNDGVYFAS | L68 | DGADVTKI | L47; L25 | VVKRHTFSNY | L14; L10; L26 |
| 1010 | FNICQAVTA | L50 | DGISQYSL | L23; L25 | VVLLILMTAR | L15; L21; L17 |
| 1011 | FNKWGKARLY | L14 | DGNAAISDY | L14; L29 | VVLSFELLH | L12; L7; L6 |
| 1012 | FNLVAMKYNY | L12 | DGYFKIYSK | L23; L19 | VVNAANVY | L26; L18; L14 |
| 1013 | FNPETNILLNV | L5 | DIAKKPTETI | L10; L11 | VVNARLRAK | L6; L7; L13 |
| 1014 | FNPPALQDAY | L12 | DIASTDTCF | L11; L10 | VVNPVMEPIY | L12; L14; L31 |
| 1015 | FNVAITRAKV | L20 | DILGPLSAQ | L10; L11 | VVNVVTTKI | L24; L49; L48 |
| 1016 | FNYLKSPNF | L59 | DILRVYANL | L10; L11 | VVPGLPGTI | L5; L57; L9 |
| 1017 | FPDLNGDV | L68 | DIPGIPKDM | L11; L57 | VVREFLTR | L15; L21; L17 |
| 1018 | FPFNKWGKAR | L50 | DIQLLKSAY | L14; L29 | VVRQCSGVTF | L26; L52; L53 |
| 1019 | FPFNKWGKARLY | L31 | DIVKTDGTL | L10; L11 | VVRSIFSR | L15; L21; L17 |
| 1020 | FPKSDGTGTI | L49 | DIYNDKVAGF | L11; L10 | VVRSIFSRTL | L13; L39; L55 |
| 1021 | FPLQSYGF | L31 | DKFPVLHDI | L47; L20 | VVYCPRHV | L48; L47; L64 |
| 1022 | FPLQSYGFQP | L50 | DKLQFTSL | L25; L23 | VVYCPRHVI | L16; L47; L39 |
| 1023 | FPNITNLC | L47 | DKVFRSSVL | L25; L44 | VYAADPAM | L71; L72; L66 |
| 1024 | FPNITNLCP | L50 | DLEGKQGNF | L11; L10 | VYANLGERV | L9; L8; L72 |
| 1025 | FPNTYLEG | L50 | DLGDELGTD | L10; L11 | VYCPRHVIC | L8; L66; L9 |
| 1026 | FPPTEPKKD | L49 | DLNDFVSDA | L3; L11 | VYDDGARRVW | L9; L62; L52 |
| 1027 | FPQSAPHGVVFL | L31 | DLNQLTGY | L11; L26 | VYDNKLKAH | L62; L71; L72 |
| 1028 | FPSGKVEG | L50 | DLQDLKWARF | L11; L10 | VYDYLVSTQEF | L8; L9; L62 |
| 1029 | FQEKDEDDNL | L34 | DLQELGKYEQY | L11; L10 | VYLAVFDKN | L9; L8; L12 |
| 1030 | FQQFGRDI | L48 | DLSVVNARL | L20; L4 | VYLKHGGGV | L72; L71; L8 |
| 1031 | FQSASKII | L48 | DLSVVNARLR | L19; L17 | VYLPYPDPS | L9; L72; L71 |
| 1032 | FQTRAGCLI | L24 | DLTKPYIKW | L10; L49 | VYMPASWV | L72; L71; L62 |
| 1033 | FQTVKPGNF | L58 | DLVYALRHF | L10; L11 | VYMPASWVMR | L15; L9; L21 |
| 1034 | FQVTIAEI | L48 | DLYDKLQFTSL | L10; L11 | VYPVASPNE | L9; L71; L72 |
| 1035 | FQVTIAEILL | L44 | DLYKLMGHF | L11; L10 | VYPVASPNEC | L72; L9; L71 |
| 1036 | FRIDGDMV | L35 | DMSKFPLKLR | L17; L19 | VYQLRARSV | L72; L71; L64 |
| 1037 | FRIDGDMVPH | L30 | DMVDTSLSGF | L10; L11 | VYSDVENPH | L72; L9; L71 |
| 1038 | FRIDGDMVPHI | L34 | DMVPHISRQ | L11; L10 | VYSVIYLYLTF | L9; L8; L12 |
| 1039 | FRKMAFPSG | L66 | DMVPHISRQR | L19; L17 | VYYGNALDQ | L71; L72; L8 |
| 1040 | FRLTLGVY | L66 | DNINLHTQV | L25; L20 | VYYHKNNKSW | L9; L8; L71 |
| 1041 | FRLTLGVYD | L30 | DNLLEILQK | L17; L7 | WAHGFELTSM | L58; L43; L59 |
| 1042 | FRRAFGEY | L66 | DPAQLPAPR | L19; L17 | WAKRNIKPV | L70; L58; L69 |
| 1043 | FRVQPTES | L66 | DPLQPELDSF | L31; L47 | WEVGKPRPP | L38; L46; L42 |
| 1044 | FRYMNSQGLL | L30 | DPLSETKCTL | L32; L23 | WFFSNYLKR | L17; L15; L19 |
| 1045 | FSALEPLVD | L69 | DPNFKDQV | L23; L47 | WFLAYILFTRF | L8; L12; L9 |
| 1046 | FSGYLKLTD | L65 | DQIGYYRRA | L11; L24 | WFLLLSVCL | L71; L72; L62 |
| 1047 | FSHSQLGGLHL | L60 | DQLTPTWRV | L24; L48 | WFLNRFTTTL | L71; L8; L72 |
| 1048 | FSLELQDH | L59 | DQLTPTWRVY | L27; L14 | WHHSIGFDY | L14; L12; L43 |
| 1049 | FSNVTWFHA | L50 | DRAMPNML | L25; L35 | WLDDDSQQT | L5; L2; L1 |
| 1050 | FSNVTWFHAI | L61 | DRDLYDKL | L25; L35 | WLDDDSQQTV | L5; L2; L24 |
| 1051 | FSNYLKRRVV | L73 | DRDLYDKLQF | L34; L35 | WLKQLIKVTL | L23; L3; L56 |
| 1052 | FSSLPSYAA | L50 | DRELHLSW | L25; L35 | WLPTGTLL | L57; L74; L62 |
| 1053 | FSSNVANYQK | L19 | DRLNQLESK | L30; L25 | WSYSGQSTQL | L60; L55; L53 |
| 1054 | FSYVGCHNK | L7 | DRVVFVLWA | L25; L35 | WVMRIMTW | L10; L54; L52 |
| 1055 | FTDGVCLFWN | L1 | DRYPANSIVC | L25; L35 | WWTAFVTNV | L20; L3; L64 |
| 1056 | FTEERLKLFD | L1 | DSAEVAVKMF | L10; L11 | YAADPAMHAA | L50; L67; L61 |
| 1057 | FTGNLQSNHDLY | L1 | DSGFAAYSR | L19; L17 | YAAVINGDRW | L52; L49; L54 |
| 1058 | FTIGTVTLKQ | L11 | DSIIIGGAK | L19; L11 | YACWHHSIG | L59; L69; L70 |
| 1059 | FTPLIQPI | L57 | DSIIIGGAKL | L20; L11 | YAFASEAAR | L19; L17; L31 |
| 1060 | FTPLIQPIGAL | L57 | DSSQGSEY | L1; L18 | YAFASEAARV | L20; L47; L58 |
| 1061 | FTRGVYYPDK | L13 | DSVEEVLSE | L10; L11 | YAKPFLNK | L13; L59; L70 |
| 1062 | FTRSTNSRIK | L13 | DSYFVVKR | L19; L17 | YAKPFLNKVV | L70; L69; L58 |
| 1063 | FTSDLATN | L59 | DTANPKTPKY | L11; L10 | YALRHFDEG | L67; L59; L61 |
| 1064 | FTVEKGIY | L1 | DTDFVNEFYAY | L1; L18 | YALVYFLQS | L59; L61; L47 |
| 1065 | FTYASALW | L54 | DTDLTKPY | L1; L18 | YANLGERVR | L17; L19; L32 |
| 1066 | FVCNLLLLFV | L20 | DTFNGECPNF | L10; L11 | YANRNRFLYI | L47; L49; L73 |
| 1067 | FVDDIVKTDGTL | L68 | DTGVEHVTFF | L10; L11 | YANSVENI | L47; L70; L49 |
| 1068 | FVDGVPFVVST | L68 | DTIANYAKP | L11; L10 | YATHSDKF | L58; L49; L54 |
| 1069 | FVDRQTAQ | L68 | DTIANYAKPF | L10; L11 | YCIPYNSV | L28; L47; L45 |
| 1070 | FVEIIKSQDL | L68 | DTLKEILVT | L10; L11 | YCQVHGNAH | L71; L72; L59 |
| 1071 | FVETVKGLD | L1 | DTLRVEAFEY | L12; L10 | YDDGARRV | L63; L33; L62 |
| 1072 | FVFLVLLP | L50 | DTPKGPKV | L11; L47 | YDFTEERL | L33; L65; L29 |
| 1073 | FVIRGDEV | L67 | DTPKGPKVK | L19; L11 | YDKLQFTSL | L23; L37; L33 |
| 1074 | FVLTSHTV | L47 | DTPKGPKVKY | L11; L10 | YDKLVSSF | L33; L43; L23 |
| 1075 | FVMMSAPPA | L50 | DTPNNTDFSR | L19; L17 | YDYLVSTQEF | L33; L43; L27 |
| 1076 | FVNLDNLRA | L50 | DTSLSGFKL | L20; L19 | YEDQDALF | L62; L33; L63 |
| 1077 | FVNLKQLPFFY | L12 | DTTDAVRDP | L11; L10 | YEKLKPVLD | L45; L46; L23 |
| 1078 | VNLKQLPFFYY | L12 | DTWFSQRGGSY | L11; L14 | YELKHGTF | L33; L29; L37 |
| 1079 | FVRATATI | L48 | DVDTDFVNEFY | L1; L18 | YENAFLPF | L29; L33; L59 |
| 1080 | FVSDADSTLI | L20 | DVEGCHATR | L19; L17 | YENAFLPFAM | L29; L36; L40 |
| 1081 | FVSEETGTLI | L20 | DVENPHLMGW | L10; L11 | YENQKLIA | L46; L38; L42 |
| 1082 | FVSGNCDVVI | L32 | DVFHLYLQYI | L20; L10 | YENQKLIANQF | L29; L40; L41 |
| 1083 | FVSLAIDA | L50 | DVNLHSSR | L17; L19 | YEQYIKWPWY | L41; L29; L40 |
| 1084 | FVSNGTHWFV | L20 | DVNLHSSRL | L11; L10 | YESLRPDTRY | L29; L40; L41 |
| 1085 | FVTDTPKGPKV | L20 | DVRETMSYL | L11; L10 | YEYGTEDDY | L29; L14; L27 |
| 1086 | FVTNVNASS | L20 | DVRETMSYLF | L11; L10 | YFAVHFISN | L71; L72; L46 |
| 1087 | FVVEVVDK | L19 | DVSFLAHIQW | L19; L10 | YFKNHTSPD | L71; L43; L72 |
| 1088 | FVVFLLVT | L59 | DVTQLYLGGM | L11; L10 | YFLCWHTNC | L71; L72; L8 |
| 1089 | FVVFLLVTLA | L50 | DVVECLKLS | L11; L10 | YFLCWHTNCY | L72; L71; L12 |
| 1090 | FVVKRHTFS | L74 | DVVECLKLSHQ | L10; L11 | YFNKKDWYDF | L8; L9; L12 |
| 1091 | FVVKRHTFSNY | L10 | DVVNQNAQA | L11; L20 | YFNSVCRL | L62; L71; L72 |
| 1092 | FVVPGLPG | L61 | DVVQEGVLTA | L11; L10 | YFNSVCRLM | L62; L71; L72 |
| 1093 | FVVPGLPGTILR | L19 | DVVQEGVLTAV | L11; L20 | YFSGAMDTTSY | L71; L72; L12 |
| 1094 | FVVSTGYHFREL | L60 | DVVRQCSGVTF | L11; L10 | YFTQSRNL | L71; L72; L62 |
| 1095 | FYDAQPCSD | L62 | DVVYRAFDIY | L10; L11 | YFVKIGPER | L17; L19; L8 |
| 1096 | FYDFAVSKGFF | L62 | DWYDFVEN | L25; L29 | YFYTSKTTVA | L71; L72; L46 |
| 1097 | FYFYTSKT | L71 | DYDCVSFCY | L12; L1 | YFYYLGTGP | L72; L71; L46 |
| 1098 | FYGPFVDRQ | L66 | DYIATNGPL | L72; L71 | YHFRELGV | L28; L35; L48 |
| 1099 | FYILPSIISN | L72 | DYIINLII | L47; L25 | YHNESGLKT | L28; L35; L34 |
| 1100 | FYKENSYTTTI | L9 | DYPKCDRAM | L72; L71 | YHNESGLKTI | L34; L28; L35 |
| 1101 | FYKGVITHD | L66 | DYTEISFM | L72; L62 | YIATNGPLK | L6; L7; L19 |
| 1102 | FYLTNDVS | L71 | DYTEISFMLW | L9; L8 | YIDINGNLHP | L1; L68; L18 |
| 1103 | FYPPDEDEEE | L66 | DYVYNPFMI | L8; L9 | YILFTRFFY | L12; L14; L1 |
| 1104 | FYSKWYIR | L17 | EAALCTFLL | L20; L74 | YKHYTPSF | L66; L27; L59 |
| 1105 | FYSYATHSDKF | L9 | EAANFCALIL | L69; L20 | YKIEELFY | L27; L66; L12 |
| 1106 | FYVYSRVK | L71 | EAEVQIDRL | L32; L63 | YKKDNSYF | L58; L27; L66 |
| 1107 | GAAAYYVGYL | L74 | EAGVCVSTSGR | L19; L17 | YKLNVGDYF | L27; L9; L12 |
| 1108 | GAALQIPFA | L50 | EAMYTPHTVL | L67; L32 | YKQFDTYNL | L44; L66; L34 |
| 1109 | GADLKSFD | L63 | EAPFLYLYAL | L57; L20 | YLALYNKYKY | L12; L1; L14 |
| 1110 | GADVTKIKP | L63 | EAPLVGTPV | L57; L20 | YLCFLAFLLF | L12; L9; L1 |
| 1111 | GAISSVLND | L49 | EASKKPRQK | L19; L17 | YLFDESGEFK | L6; L2; L13 |
| 1112 | GAISSVLNDIL | L67 | EASKKPRQKR | L17; L19 | YLFDESGEFKL | L2; L4; L5 |
| 1113 | GALDISASI | L44 | EAVKTQFNYY | L11; L10 | YLKRRVVF | L23; L26; L27 |
| 1114 | GATTCGYL | L74 | EAVMYMGTL | L10; L61 | YLKSPNFSK | L13; L6; L43 |
| 1115 | GAVILRGHLR | L19 | ECFDKFKV | L20; L24 | YLKSPNFSKL | L3; L10; L5 |
| 1116 | GAWNIGEQKS | L24 | ECPNFVFPL | L57; L20 | YLPQNAVVKI | L5; L3; L2 |
| 1117 | GCDVTDVTQL | L34 | ECVLGQSKR | L19; L17 | YLTNDVSF | L43; L26; L74 |
| 1118 | GCDVTDVTQLY | L18 | EDLLFNKV | L33; L48 | YLVSTQEFRY | L12; L14; L1 |
| 1119 | GCINANQV | L48 | EEAIRHVR | L29; L42 | YMPASWVM | L57; L66; L71 |
| 1120 | GCVPLNII | L48 | EEAKTVLKKC | L41; L40 | YNDKVAGF | L62; L33; L63 |
| 1121 | GCVPLNIIPL | L44 | EEEEFEPSTQY | L41; L40 | YNGSPSGVY | L65; L14; L12 |
| 1122 | GDAALALL | L33 | EEEFEPSTQY | L41; L40 | YPDKVFRSS | L32; L49; L39 |
| 1123 | GDAALALLL | L40 | EEFEPSTQYE | L41; L40 | YPDKVFRSSV | L32; L22; L39 |
| 1124 | GDCEEEEF | L33 | EEGDCEEEEF | L40; L41 | YPKLQSSQA | L51; L50; L22 |
| 1125 | GDELGTDPY | L18 | EEIAIILASFS | L40; L41 | YPNMFITR | L50; L17; L47 |
| 1126 | GDELKINAA | L46 | EELDKYFKNH | L41; L40 | YPNMFITRE | L49; L32; L50 |
| 1127 | GDFIQTTP | L65 | EELFYSYAT | L42; L40 | YQIGGYTEKW | L49; L10; L24 |
| 1128 | GDFLHFLP | L33 | EELKKLLEQW | L40; L41 | YQPPQTSI | L48; L24; L57 |
| 1129 | GDFLHFLPR | L7 | EESSAKSA | L42; L38 | YQVNGYPNM | L58; L28; L61 |
| 1130 | GDFLHFLPRV | L33 | EESSAKSASV | L42; L38 | YQVNGYPNMF | L34; L27; L58 |
| 1131 | GDFVKATC | L33 | EESSAKSASVY | L41; L40 | YRENGIGV | L25; L35; L64 |
| 1132 | GDIAARDL | L37 | EETFKLSYGI | L40; L41 | YRENGIGVTQ | L30; L35; L34 |
| 1133 | GDMVPHISR | L15 | EETGLLMP | L42; L46 | YRVVVLSFEL | L66; L30; L35 |
| 1134 | GDQFKHLI | L33 | EETKFLTENL | L41; L40 | YSGVVTTV | L48; L73; L70 |
| 1135 | GDSAEVAV | L33 | EETRKLMP | L42; L46 | YSVIYLYLTF | L52; L54; L10 |
| 1136 | GDSCNNYM | L33 | EETRKLMPV | L42; L45 | YTDFATSA | L1; L68; L63 |
| 1137 | GDSEVVLK | L33 | EEVLSEARQ | L41; L40 | YTEISFML | L1; L68; L60 |
| 1138 | GDSEVVLKKL | L37 | EEVLSEARQHL | L40; L41 | YTERSEKSY | L1; L18; L14 |
| 1139 | GDSVEEVL | L33 | EEVQELYSPI | L41; L40 | YTRYVDNNF | L53; L52; L58 |
| 1140 | GDVRETMSY | L27 | EEVQELYSPIF | L40; L41 | YTSNPTTFHL | L20; L60; L61 |
| 1141 | GDVVQEGV | L33 | EEVTTTLEETKF | L40; L41 | YVMHANYIF | L58; L12; L31 |
| 1142 | GDYFVLTSH | L43 | EEVVLKTGDL | L41; L40 | YVPAQEKNF | L5; L10; L12 |
| 1143 | GEGSEGLNDNL | L36 | EFEPSTQYEY | L12; L1 | YVRITGLY | L1; L14; L26 |
| 1144 | GEGSEGLNDNLL | L36 | EFGATSAAL | L72; L71 | YVRNLQHRLY | L12; L14; L13 |
| 1145 | GEIPVAYR | L45 | EFIERYKLEGY | L11; L10 | YVVDDPCPI | L20; L67; L49 |
| 1146 | GEQKSILSPL | L36 | EFLRDGWEI | L8; L9 | YVVDDPCPIHFY | L11; L14; L12 |
| 1147 | GETFVTHSKGL | L36 | EFTPFDVVR | L17; L19 | YVWKSYVHVV | L2; L5; L3 |
| 1148 | GETLPTEVLT | L36 | EFVFKNIDGY | L11; L10 | YVYNPFMID | L61; L60; L59 |
| 1149 | GEVFNATR | L45 | EFYEAMYTP | L42; L8 | YWVPRASA | L50; L46; L51 |
| 1150 | GEVPVSIINNTV | L36 | EGYAFEHI | L48; L47 | YYFMRFRRAF | L71; L72; L9 |
| 1151 | GEYSHVVAFNTL | L36 | EGYAFEHIV | L48; L47 | YYKKDNSY | L66; L72; L71 |
| 1152 | GEYTFEKGDY | L41 | EHEHEIAW | L35; L28 | YYRRATRRI | L8; L48; L9 |
| 1153 | GFDVEGCH | L62 | EHSWNADL | L35; L28 | YYSDSPCES | L66; L72; L9 |
| 1154 | GFDYVYNP | L62 | EHSWNADLYKL | L34; L35 | AACRKVQHM | L70; L69; L58; |
| | | | | | | L63 |
| 1155 | GFDYVYNPF | L62 | EIAWYTER | L17; L19 | AADLDDESK | L68; L7; L63; L18 |
| 1156 | GFDYVYNPFM | L62 | EIKDATPSDF | L10; L11 | AAFATAQEAY | L31; L27; L14; |
| | | | | | | L21 |
| 1157 | GFFKEGSSVEL | L44 | EIKESVQTFF | L10; L11 | AAIFYLITP | L69; L42; L38; |
| | | | | | | L46 |
| 1158 | GFLFLTWI | L48 | EILGTVSWNLR | L19; L17 | AAKAYKDY | L43; L70; L58; |
| | | | | | | L14 |
| 1159 | GFQPTNGV | L48 | EILLIIMR | L17; L19 | AAKKNNLPF | L58; L43; L70; |
| | | | | | | L52 |
| 1160 | GFSALEPL | L72 | EIPKEEVKPF | L10; L11 | AARVVRSI | L48; L55; L70; |
| | | | | | | L47 |
| 1161 | GFSTGVNLV | L64 | EIPVAYRKV | L11; L10 | AATRGATVV | L69; L70; L58; |
| | | | | | | L24 |
| 1162 | GFTLKNTV | L48 | EITVATSR | L17; L19 | AAVDALCEK | L7; L19; L67; L69 |
| 1163 | GGAYTRYV | L48 | EIVDTVSALV | L20; L11 | ADAVIKTL | L33; L37; L25; |
| | | | | | | L23 |
| 1164 | GGDGKMKDL | L63 | EIVDTVSALVY | L11; L10 | ADFDTWFSQR | L15; L21; L7; L19 |
| 1165 | GGFNFSQI | L48 | EIYQAGSTP | L10; L11 | ADSKIVQL | L33; L37; L56; |
| | | | | | | L65 |
| 1166 | GGIVAIVV | L48 | EKFKEGVEFL | L44; L20 | AEAELAKNVSL | L36; L41; L40; |
| | | | | | | L37 |
| 1167 | GGIVAIVVT | L44 | ELAMDEFIERY | L11; L10 | AECTIFKDA | L42; L46; L38; |
| | | | | | | L40 |
| 1168 | GGKIVNNWL | L44 | ELGDVRETMSY | L10; L11 | AEILLIIMRTF | L41; L40; L37; |
| | | | | | | L29 |
| 1169 | GGKPCIKV | L48 | ELKFNPPAL | L23; L3 | AEIRASANLA | L38; L42; L46; |
| | | | | | | L40 |
| 1170 | GGNYNYLYR | L15 | ELKKLLEQW | L10; L11 | AESHVDTDL | L36; L37; L41; |
| | | | | | | L40 |
| 1171 | GGQPITNCV | L48 | ELLQNGMNGR | L17; L19 | AEWFLAYI | L45; L33; L48; |
| | | | | | | L37 |
| 1172 | GGRFVLALL | L44 | ELSPVALRQ | L24; L20 | AFATAQEAY | L72; L71; L12; |
| | | | | | | L14 |
| 1173 | GGSVAIKI | L48 | ELSRVLGLK | L19; L6 | AFAVDAAKAY | L14; L72; L71; |
| | | | | | | L12 |
| 1174 | GGTTEMLAK | L7 | ELTGHMLDMY | L11; L10 | AFEKMVSL | L62; L72; L71; |
| | | | | | | 3L6 |
| 1175 | GGVAGALNK | L7 | ELYHYQECV | L24; L20 | AFLPFAMGI | L71; L72; L8; L9 |
| 1176 | GHFAWWTA | L28 | ENFNQHEV | L48; L20 | AFNTLLFL | L62; L71; L72; |
| | | | | | | L73 |
| 1177 | GHFAWWTAFV | L28 | EPEEHVQI | L49; L25 | AFYILPSI | L48; L71; L72; |
| | | | | | | 7L4 |
| 1178 | GHHLGRCDI | L34 | EPKLGSLV | L23; L51 | AGNGGDAAL | L44; L57; L56; |
| | | | | | | L61 |
| 1179 | GHMLDMYSVM | L28 | EPKLGSLVVR | L17; L19 | AGSKSPIQY | L26; L27; L14; |
| | | | | | | L18 |
| 1180 | GHMLDMYSVML | L28 | EPTTTTSV | L23; L50 | AGTDTTITV | L24; L69; L48; L2 |
| 1181 | GHSMQNCV | L28 | EQLDFIDTK | L24; L7 | AHISTIGVC | L35; L28; L34; |
| | | | | | | L72 |
| 1182 | GHSMQNCVLKL | L28 | ERFVSLAI | L25; L35 | AHSCNVNRF | L34; L35; L28; |
| | | | | | | L27 |
| 1183 | GHTDLMAA | L28 | ERHSLSHFV | L35; L64 | AIDAYPLTK | L6; L7; L18; L68 |
| 1184 | GHTDLMAAYV | L28 | ERIDKVLNE | L30; L35 | AIMTRCLAV | L23; L73; L3; L4 |
| 1185 | GHVETFYPK | L7 | ERIDKVLNEK | L30; L11 | AISDYDYY | L14; L18; L1; L58 |
| 1186 | GHVMVELV | L28 | ESELVIGAV | L20; L1 | AISDYDYYRY | L12; L14; L18; |
| | | | | | | L16 |
| 1187 | GIDLDEWSMATY | L18 | ESIVRFPNI | L48; L20 | ALALLLLDR | L21; L6; L7; L15 |
| 1188 | GIKIQEGVVDY | L26 | ESLRPDTRY | L11; L14 | ALCEKALKYL | L3; L2; L4; L5 |
| 1189 | GIPKDMTY | L26 | ETAHSCNVNRF | L11; L10 | ALDQAISM | L63; L68; L62; |
| | | | | | | L18 |
| 1190 | GIVAIVVTC | L19 | ETAQNSVR | L17; L19 | ALLKTVQF | L26; L27; L33; |
| | | | | | | L74 |
| 1191 | GIVNNTVY | L26 | ETFVTHSK | L19; L17 | ALNHTKKWKY | L14; L12; L18; |
| | | | | | | L16 |
| 1192 | GIYQTSNF | L26 | ETFVTHSKGL | L10; L20 | ALNLGETFV | L2; L4; L24; L3 |
| 1193 | GKASCTLSE | L44 | ETFYPKLQSS | L20; L10 | ALYYPSARI | L24; L45; L2; L3 |
| 1194 | GKASCTLSEQL | L44 | ETICAPLTVF | L10; L11 | AMDEFIER | L15; L21; L62; |
| | | | | | | L68 |
| 1195 | GKFCLEASFNY | L27 | ETKDVVECLKL | L11; L10 | ANGQVFGLY | L14; L12; L18; |
| | | | | | | L40 |
| 1196 | GKGYHLMSF | L27 | ETLVTMPLGY | L11; L12 | APAHISTIGV | L56; L22; L51; |
| | | | | | | L39 |
| 1197 | GKIADYNY | L27 | ETMSYLFQHA | L20; L10 | APHGHVMVEL | L56; L39; L22; |
| | | | | | | L32 |
| 1198 | GKIADYNYKL | L44 | ETNILLNV | L48; L73 | APPAQYEL | L56; L39; L22; |
| | | | | | | L66 |
| 1199 | GKIQDSLSS | L27 | ETQALPQR | L17; L19 | AQNSVRVLQK | L7; L6; L13; L24 |
| 1200 | GKIQDSLSST | L27 | ETSWQTGDF | L11; L10 | AQYTSALL | L28; L48; L44; |
| | | | | | | L27 |
| 1201 | GKIVNNWL | L44 | ETVKGLDY | L11; L1 | ARDGCVPL | L35; L66; L63; |
| | | | | | | L68 |
| 1202 | GKIVNNWLKQL | L44 | ETVKGLDYKAF | L10; L11 | ARDLSLQF | L35; L33; L65; |
| | | | | | | L62 |
| 1203 | GKMKDLSPRWY | L27 | ETYFTQSR | L17; L19 | ARHINAQVA | L35; L25; L46; |
| | | | | | | L28 |
| 1204 | GKPCIKVAT | L44 | EVAVKMFDAY | L10; L11 | ARNGVLITE | L30; L65; L66; |
| | | | | | | L35 |
| 1205 | GKPREQIDGY | L27 | EVEKGVLPQL | L11; L20 | ARTRSMWSF | L66; L30; L35; |
| | | | | | | 4L3 |
| 1206 | GKPVPYCY | L66 | EVFNATRFA | L20; L11 | ASAVVLLI | L73; L48; L55; |
| | | | | | | L24 |
| 1207 | GKSHFAIGL | L44 | EVGFVVPGL | L20; L11 | ASFRLFARTR | L15; L19; L21; |
| | | | | | | L55 |
| 1208 | GKVEGCMVQ | L27 | EVGHTDLMAAY | L11; L10 | ASFYYVWKSY | L43; L16; L14; |
| | | | | | | 2L5 |
| 1209 | GKVEGCMVQV | L27 | EVITFDNL | L20; L74 | ASKKPRQKR | L15; L13; L21; |
| | | | | | | L17 |
| 1210 | GKYEQYIKWP | L27 | EVITFDNLKTL | L10; L11 | ASMPTTIAK | L7; L6; L13; L43 |
| 1211 | GKYEQYIKWPW | L27 | EVKPFITESK | L11; L10 | ASSSEAFLI | L55; L24; L53; |
| | Y | | | | | L54 |
| 1212 | GKYVQIPTTC | L27 | EVLSDREL | L23; L55 | ATAEAELAK | L7; L6; L19; L13 |
| 1213 | GLAAIMQLFF | L12 | EVLSEARQHL | L10; L20 | ATCELYHY | L18; L1; L14; L54 |
| 1214 | GLCVDIPGIPK | L6 | EVNEFACVV | L20; L11 | ATEETFKL | L63; L1; L18; L68 |
| 1215 | GLFCLLNRYF | L12 | EVNSFSGYLKL | L11; L10 | ATLQAIASEF | L52; L53; L16; |
| | | | | | | L10 |
| 1216 | GLHLLIGL | L23 | EVPANSTVLSF | L11; L10 | ATNGPLKV | L73; L70; L48; |
| | | | | | | L24 |
| 1217 | GLHLLIGLA | L24 | EVPVAIHAD | L10; L11 | ATNNLVVM | L70; L73; L59; |
| | | | | | | L58 |
| 1218 | GLHLLIGLAK | L6 | EVPVSIINN | L17; L11 | ATNYDLSVV | L73; L70; L58; |
| | | | | | | L18 |
| 1219 | GLHPTQAP | L43 | EVQIDRLITGR | L17; L19 | ATRFASVY | L18; L14; L26; L1 |
| 1220 | GLKTILRKG | L3 | EVTPSGTW | L10; L54 | ATRGATVVI | L53; L70; L52; |
| | | | | | | L10 |
| 1221 | GLKTLATHGL | L3 | EVTPSGTWLTY | L11; L10 | ATSRTLSY | L18; L1; L54; L14 |
| 1222 | GLLMPLKAPK | L6 | EVVDKYFDCY | L10; L11 | AVDALCEKAL | L68; L5; L67; L63 |
| 1223 | GLMWLSYF | L26 | EVVENPTIQ | L11; L20 | AYANRNRF | L9; L71; L72; L8 |
| 1224 | GLNGYTVEE | L2 | EWFLAYILFTR | L17; L19 | AYANSVENI | L9; L8; L72; L34 |
| 1225 | GLPGTILR | L21 | EWSMATYYLF | L9; L8 | AYFNMVYM | L71; L72; L62; |
| | | | | | | L66 |
| 1226 | GLTGTGVL | L74 | EYCPIFFI | L9; L8 | AYFNMVYMP | L42; L71; L66; |
| | | | | | | L72 |
| 1227 | GLTGTGVLT | L2 | EYHDVRVVLDF | L8; L9 | AYIICISTK | L13; L72; L7; L71 |
| 1228 | GLVAEWFL | L74 | EYHNESGL | L71; L72 | AYIICISTKHF | L8; L9; L72; L40 |
| 1229 | GLVAEWFLA | L2 | EYKGPITDV | L17; L11 | AYITGGVVQ | L71; L72; L8; L9 |
| 1230 | GLWLDDVVYC | L2 | EYSHVVAFNTL | L8; L9 | AYNGYLTSS | L72; L71; L15; L8 |
| 1231 | GLYRKCVKS | L24 | EYYHTTDPSF | L9; L8 | AYSNNSIAI | L72; L9; L71; L8 |
| 1232 | GLYRKCVKSR | L21 | FADDLNQLTG | L67; L68 | AYVDNSSLTI | L8; L9; L71; L72 |
| 1233 | GMSRIGMEV | L24 | FALTCFSTQ | L59; L61 | AYVNTFSSTF | L8; L9; L71; L72 |
| 1234 | GMVLGSLAA | L46 | FAMGIIAMSA | L50; L61 | AYYFMRFRR | L15; L17; L21; |
| | | | | | | L12 |
| 1235 | GNAAISDYDY | L12 | FASTEKSNI | L49; L70 | CCNIVNVSL | L56; L44; L32; |
| | | | | | | L35 |
| 1236 | GNCDVVIGI | L24 | FATSACVLA | L50; L69 | CEFCGTENL | L37; L36; L29; |
| | | | | | | L45 |
| 1237 | GNFGDQEL | L65 | FCDLKGKY | L1; L18 | CFSTASDTY | L14; L72; L71; |
| | | | | | | L12 |
| 1238 | GNFKVTKGK | L13 | FCLEASFNY | L12; L31 | CLHVVGPNV | L2; L24; L4; L5 |
| 1239 | GNFYGPFV | L48 | FCNDPFLGVYY | L31; L12 | CLVGLMWLSY | L12; L16; L10; |
| | | | | | | L11 |
| 1240 | GNFYGPFVD | L65 | FDAYVNTF | L33; L29 | CSNLLLQY | L18; L59; L1; L48 |
| 1241 | GNVQLRVI | L48 | FDESGEFKL | L32; L63 | CTSVVLLSV | L73; L20; L69; |
| | | | | | | L70 |
| 1242 | GPDGYPLEC | L49 | FEKMVSLL | L33; L23 | CVDIPGIPK | L7; L6; L13; L68 |
| 1243 | GPEAGLPY | L18 | FEKMVSLLSV | L45; L46 | CVPLNIIPL | L57; L5; L74; L64 |
| 1244 | GPEQTQGNF | L49 | FELDERIDKVL | L40; L41 | CVRGTTVLL | L74; L58; L13; |
| | | | | | | L56 |
| 1245 | GPITDVFYK | L7 | FERDISTEIY | L43; L29 | CYLATALL | L71; L72; L9; L62 |
| 1246 | GPKQASLNGV | L51 | FEYYHTTD | L25; L45 | CYLATALLTL | L8; L9; L71; L72 |
| 1247 | GPKQASLNGVTL | L56 | FFGMSRIGM | L72; L71 | CYTPSKLIEY | L12; L72; L66; |
| | | | | | | L71 |
| 1248 | GPKVKYLY | L23 | FFITGNTLQ | L71; L72 | DAAKAYKDY | L10; L11; L58; |
| | | | | | | L49 |
| 1249 | GPLKVGGSCV | L51 | FFITGNTLQC | L71; L72 | DAYNMMISA | L47; L50; L25; |
| | | | | | | L69 |
| 1250 | GPLKVGGSCVL | L56 | FFIYNKIVD | L72; L71 | DDTLRVEAF | L33; L29; L23; |
| | | | | | | L25 |
| 1251 | GPLVRKIF | L23 | FFKEGSSVE | L71; L72 | DEDDNLIDSY | L29; L41; L40; L1 |
| 1252 | GPNVNKGED | L49 | FFLYENAFLPF | L12; L8 | DEFSSNVAN | L29; L46; L25; |
| | | | | | | L41 |
| 1253 | GQAITVTP | L48 | FFYYSDSP | L71; L72 | DEISMATNY | L29; L41; L11; |
| | | | | | | L40 |
| 1254 | GQINDMILS | L24 | FFYYSDSPC | L71; L72 | DELTGHML | L25; L29; L23; |
| | | | | | | L37 |
| 1255 | GQIVTCAKE | L24 | FGAGAALQI | L64; L47 | DEPTTTTSV | L45; L29; L57; |
| | | | | | | L37 |
| 1256 | GQKTYERHSL | L44 | FGATSAAL | L61; L59 | DFMSLSEQL | L8; L71; L9; L72 |
| 1257 | GQQDGSEDN | L24 | FGLVAEWFL | L61; L44 | DISGINASV | L20; L11; L28; |
| | | | | | | L19 |
| 1258 | GQQGEVPV | L24 | FHPLADNKF | L34; L62 | DIVEEAKKV | L11; L10; L20; |
| | | | | | | L24 |
| 1259 | GQQGEVPVS | L24 | FIAGLIAIVMV | L20; L2 | DKFKVNSTL | L44; L25; L27; |
| | | | | | | L35 |
| 1260 | GQQGEVPVSI | L24 | FIDTKRGV | L63; L68 | DKVAGFAKF | L10; L11; L27; |
| | | | | | | L29 |
| 1261 | GQQQQGQTVTK | L7 | FIEDLLFNKV | L5; L20 | DLKGKYVQI | L23; L10; L11; L3 |
| 1262 | GQQQTTLKG | L24 | FIERYKLEGY | L1; L18 | DLPQGFSAL | L57; L10; L11; L5 |
| 1263 | GQSTQLGIEFL | L44 | FIKGLNNLNR | L17; L21 | DLQDLKWAR | L17; L19; L21; |
| | | | | | | L15 |
| 1264 | GQTFSVLAC | L24 | FIPMDSTV | L57; L5 | DLQELGKY | L14; L11; L1; L10 |
| 1265 | GQTFSVLACY | L14 | FISDEVAR | L19; L17 | DLSPRWYFYY | L11; L12; L1; L18 |
| 1266 | GQTGKIADYNY | L14 | FISNSWLMW | L49; L69 | DLYDKLQF | L23; L11; L43; |
| | | | | | | L25 |
| 1267 | GQTVTKKS | L48 | FITGNTLQC | L60; L58 | DLYDKLQFT | L11; L2; L3; L10 |
| 1268 | GQVDLFRN | L24 | FITLCFTLK | L6; L7 | DMILSLLSK | L6; L19; L17; L7 |
| 1269 | GQVDLFRNAR | L15 | FIYNKIVD | L43; L23 | DNLKTLLSL | L25; L23; L8; L73 |
| 1270 | GQVFGLYKN | L24 | FKESPFEL | L66; L68 | DPCPIHFY | L49; L29; L31; |
| | | | | | | L47 |
| 1271 | GRCDIKDLPK | L30 | FKKGAKLL | L66; L64 | DPFLGVYY | L31; L29; L47; |
| | | | | | | L49 |
| 1272 | GRDIADTTD | L34 | FKLKDCVMY | L27; L31 | DPIHSLRVC | L47; L32; L49; |
| | | | | | | L31 |
| 1273 | GRFVLALLS | L30 | FKLVNKFLAL | L61; L44 | DPNFKDQVIL | L39; L32; L56; |
| | | | | | | L23 |
| 1274 | GRFVLALLSD | L30 | FKNLREFVF | L66; L27 | DPSFLGRYM | L31; L32; L49; |
| | | | | | | L22 |
| 1275 | GRLIIREN | L65 | FKYWDQTY | L27; L66 | DPVGFTLKN | L49; L11; L47; |
| | | | | | | L32 |
| 1276 | GRLIIRENN | L30 | FLALITLAT | L2; L3 | DQAISMWAL | L25; L44; L35; |
| | | | | | | L28 |
| 1277 | GRLIIRENNR | L30 | FLAYILFTRF | L12; L10 | DSNGTITV | L48; L47; L70; |
| | | | | | | L73 |
| 1278 | GRLQSLQTYV | L30 | FLEYHDVRVV | L3; L2 | DTDFVNEF | L62; L1; L68; L63 |
| 1279 | GRSGETLGVLV | L30 | FLGIITTVA | L2; L3 | DTTEAFEKM | L11; L10; L29; |
| | | | | | | L20 |
| 1280 | GRTIAFGGC | L30 | FLKKDAPYI | L2; L3 | DTTITVNVL | L10; L25; L35; |
| | | | | | | L11 |
| 1281 | GRTILGSALL | L30 | FLKKDAPYIV | L3; L2 | DTVSALVYD | L11; L20; L19; |
| | | | | | | L17 |
| 1282 | GRVDGQVD | L65 | FLKRGDKSV | L3; L2 | DTYNLWNTFTR | L19; L17; L21; L7 |
| 1283 | GRVDGQVDLFR | L30 | FLLNKEMYLKL | L4; L2 | DVDTDFVNEF | L10; L11; L68; |
| | | | | | | L49 |
| 1284 | GRWVLNNDY | L30 | FLLPSLATVAY | L12; L31 | DVKCTSVVL | L23; L10; L11; |
| | | | | | | L39 |
| 1285 | GRWVLNNDYY | L30 | FLNRVCGVSA | L2; L50 | DYFNKKDWY | L72; L12; L14; |
| | | | | | | L71 |
| 1286 | GRWVLNNDYYR | L30 | FLPFAMGII | L57; L5 | DYFVLTSH | L71; L25; L72; |
| | | | | | | L17 |
| 1287 | GRYMSALNH | L30 | FLPFFSNV | L57; L5 | DYGDAVVYR | L17; L19; L15; |
| | | | | | | L21 |
| 1288 | GRYMSALNHT | L30 | FLPFFSNVTW | L57; L5 | EAANFCAL | L67; L59; L61; |
| | | | | | | L20 |
| 1289 | GRYMSALNHTK | L30 | FLTWICLLQF | L12; L5 | EDQDALFAY | L40; L10; L14; |
| | | | | | | L29 |
| 1290 | GSCGSVGF | L54 | FLVFLGIITTV | L2; L3 | EEAALCTFL | L41; L40; L36; |
| | | | | | | L20 |
| 1291 | GSEGLNDNL | L1 | FLVLIMLII | L69; L67 | EEAALCTFLL | L41; L40; L42; |
| | | | | | | L20 |
| 1292 | GSEYDYVIF | L53 | FLVLIMLIIF | L74; L69 | EEAKTVLKK | L42; L19; L41; |
| | | | | | | L40 |
| 1293 | GSGVPVVD | L65 | FLYENAFLPFA | L2; L3 | EEEQEEDWL | L36; L41; L40; |
| | | | | | | L34 |
| 1294 | GSIHLYFDK | L7 | FMGRIRSV | L48; L64 | EEHFIETI | L45; L29; L48; |
| | | | | | | L37 |
| 1295 | GSLIYSTAA | L46 | FNGECPNFV | L73; L64 | EEKFKEGVEF | L40; L41; L42; |
| | | | | | | L37 |
| 1296 | GSLVVRCSFY | L14 | FNGVSFSTF | L59; L9 | EELDKYFKN | L41; L40; L29; |
| | | | | | | L42 |
| 1297 | GSLYVNKHAF | L52 | FNQHEVLL | L62; L74 | EELKKLLEQ | L42; L41; L40; |
| | | | | | | L46 |
| 1298 | GSNVFQTR | L15 | FNVLFSTV | L47; L48 | EEMLDNRATL | L40; L41; L36; |
| | | | | | | L42 |
| 1299 | GSRGGSQAS | L43 | FNVPMEKL | L67; L74 | EETKFLTEN | L42; L46; L29; |
| | | | | | | L45 |
| 1300 | GSSVELKHFF | L52 | FPDLNGDVVA | L50; L32 | EEVVLKTGD | L41; L42; L46; |
| | | | | | | L40 |
| 1301 | GSTPCNGV | L48 | FPFNKWGKARL | L56; L31 | EHDYQIGGY | L1; L11; L10; L18 |
| 1302 | GTCGLVEVEK | L7 | FPKSDGTGTIY | L31; L49 | EHEHEIAWY | L11; L34; L1; L10 |
| 1303 | GTDLEGNFYG | L1 | FPLCANGQV | L50; L47 | EHVQIHTI | L34; L48; L35; |
| | | | | | | L28 |
| 1304 | GTDTTITVNV | L18 | FPLKLRGTA | L50; L51 | EIIFLEGETL | L20; L10; L34; |
| | | | | | | L11 |
| 1305 | GTGVLTESNK | L7 | FPNITNLCPF | L31; L49 | EIKDTEKY | L11; L10; L26; |
| | | | | | | L43 |
| 1306 | GTHHWLLL | L73 | FPPTSFGPLV | L47; L50 | EILLIIMRTF | L10; L11; L40; |
| | | | | | | L41 |
| 1307 | GTHWFVTQRNF | L52 | FPQSAPHGVV | L50; L22 | EIVGGQIVTC | L11; L10; L19; |
| | | | | | | L20 |
| 1308 | GTILTRPLL | L13 | FPREGVFVS | L50; L31 | EKSNIIRGW | L10; L40; L41; |
| | | | | | | L49 |
| 1309 | GTKRFDNPVL | L53 | FPSGKVEGC | L49; L31 | ELAKNVSL | L23; L20; L35; |
| | | | | | | L56 |
| 1310 | GTLIVNSV | L48 | FPVLHDIGN | L50; L31 | ELGTEVNEF | L10; L9; L11; L26 |
| 1311 | GTLIVNSVLLF | L52 | FSALEPLVDL | L74; L60 | ELIRQGTDY | L11; L10; L26; |
| | | | | | | L12 |
| 1312 | GTNGTKRF | L54 | FSGAMDTTSY | L43; L1 | ELLHAPATV | L2; L4; L20; L3 |
| 1313 | GTNGTKRFD | L65 | FSNSGSDV | L58; L70 | ELNGGAYTRY | L11; L10; L14; |
| | | | | | | L26 |
| 1314 | GTNTSNQVAVLY | L18 | FSSEIIGYK | L19; L7 | ELVAELEGI | L10; L20; L24; |
| | | | | | | L11 |
| 1315 | GTRNPANNA | L13 | FSTASDTY | L1; L54 | ENFNQHEVL | L25; L20; L35; |
| | | | | | | L34 |
| 1316 | GTSTDVVY | L18 | FTALTQHGK | L19; L7 | EQYIKWPWY | L26; L27; L11; |
| | | | | | | L48 |
| 1317 | GTSTDVVYRAF | L52 | FTEERLKLFDRY | L1; L18 | ERDISTEIY | L1; L34; L35; L18 |
| 1318 | GTTEMLAK | L7 | FTIYSLLLCR | L19; L17 | ERSEAGVCV | L35; L34; L28; |
| | | | | | | L20 |
| 1319 | GTTLPKGF | L55 | FTLLLQLCTF | L52; L53 | ESKPSVEQR | L17; L19; L11; |
| | | | | | | L15 |
| 1320 | GTTSPISEHDY | L14 | FTPLVPFWI | L64; L20 | ETAHSCNVNR | L19; L17; L20; |
| | | | | | | L21 |
| 1321 | GTTTYKLNV | L73 | FTRSTNSR | L17; L21 | ETICAPLTV | L20; L73; L11; |
| | | | | | | L10 |
| 1322 | GTVSWNLREM | L10 | FTSDLATNNL | L20; L60 | ETISLAGSYK | L19; L11; L17; L7 |
| 1323 | GVAMPNLY | L18 | FTTTLNDF | L59; L58 | ETLGVLVPH | L17; L20; L7; L11 |
| 1324 | GVAPGTAVLRQ | L52 | FTVLCLTPV | L20; L59 | ETVKGLDYK | L19; L11; L20; |
| | W | | | | | L17 |
| 1325 | GVCSMTDIAK | L7 | FVCDNIKFA | L3; L69 | EVFAQVKQIYK | L19; L7; L6; L20 |
| 1326 | GVCSMTDIAKK | L7 | FVDRQTAQA | L5; L63 | EVFNATRF | L11; L10; L29; |
| | | | | | | L20 |
| 1327 | GVDIAANTVI | L68 | FVFKNIDGYFK | L6; L7 | EVNSFSGY | L11; L10; L26; |
| | | | | | | L14 |
| 1328 | GVDVELFENK | L7 | FVIRGDEVR | L19; L17 | EVPANSTVL | L10; L11; L57; |
| | | | | | | L20 |
| 1329 | GVEGFNCYF | L12 | FVLWAHGF | L59; L60 | EVVDKYFDC | L11; L10; L20; |
| | | | | | | L19 |
| 1330 | GVEHVTFF | L63 | FVNLKQLPFF | L58; L59 | EVVGDIILKP | L11; L10; L20; |
| | | | | | | L19 |
| 1331 | GVEHVTFFI | L24 | FVTHSKGL | L60; L61 | EYHDVRVV | L64; L72; L48; |
| | | | | | | L71 |
| 1332 | GVGYQPYRV | L24 | FVTHSKGLYR | L19; L17 | EYVSQPFLM | L9; L8; L17; L66 |
| 1333 | GVITHDVSSA | L3 | FVVPGLPGT | L11; L20 | FAAETLKA | L50; L69; L59; |
| | | | | | | L61 |
| 1334 | GVKHVYQL | L23 | FVVSTGYHFR | L19; L17 | FACPDGVKHV | L70; L69; L58; |
| | | | | | | L47 |
| 1335 | GVKHVYQLRAR | L15 | FWITIAYI | L62; L47 | FASFYYVWK | L7; L67; L19; L69 |
| 1336 | GVLTAVVI | L55 | FWRNTNPIQL | L62; L74 | FATAQEAY | L59; L31; L58; |
| | | | | | | L70 |
| 1337 | GVLTESNKKF | L52 | FYDFAVSKG | L62; L68 | FAVHFISNS | L69; L70; L58; |
| | | | | | | L49 |
| 1338 | GVPVVDSY | L26 | FYDFAVSKGF | L62; L9 | FAWWTAFVTN | L67; L50; L49; |
| | | | | | | L31 |
| 1339 | GVQIPCTCGK | L7 | FYEAMYTPH | L71; L72 | FCGPDGYPL | L67; L61; L66; |
| | | | | | | L60 |
| 1340 | GVSAARLT | L55 | FYFYTSKTT | L71; L72 | FCTQLNRAL | L61; L60; L64; |
| | | | | | | L32 |
| 1341 | GVSICSTMTNR | L19 | FYFYTSKTTV | L71; L72 | FDKAGQKTY | L58; L43; L14; |
| | | | | | | L70 |
| 1342 | GVSPTKLND | L65 | FYGPFVDR | L17; L66 | FDYVYNPF | L33; L59; L71; |
| | | | | | | L43 |
| 1343 | GVTFQSAVK | L7 | FYKENSYTT | L66; L9 | FEEAALCTF | L38; L41; L36; |
| | | | | | | L40 |
| 1344 | GVTRELMREL | L10 | FYLCFLAFLL | L9; L8 | FELDERIDKV | L45; L36; L38; |
| | | | | | | L37 |
| 1345 | GVVCTEIDPK | L7 | FYLITPVH | L71; L72 | FELLHAPATV | L45; L46; L38; |
| | | | | | | L36 |
| 1346 | GVVDYGARFYF | L12 | FYPPDEDE | L66; L71 | FELTSMKYF | L41; L40; L29; |
| | | | | | | L33 |
| 1347 | GVVFLHVTYV | L20 | FYRLANEC | L72; L71 | FENKTTLPV | L45; L38; L37; |
| | | | | | | L42 |
| 1348 | GVVGEGSEGL | L10 | FYSKWYIRV | L64; L62 | FEPSTQYEY | L66; L12; L40; |
| | | | | | | L41 |
| 1349 | GVVTTVMF | L74 | FYSYATHSD | L72; L71 | FERDISTEI | L45; L43; L36; |
| | | | | | | L37 |
| 1350 | GVVTTVMFL | L74 | FYTSKTTV | L71; L72 | FFPDLNGDV | L62; L72; L71; L5 |
| 1351 | GVYDYLVSTQ | L10 | FYTSKTTVASL | L71; L72 | FFSNVTWF | L72; L62; L71; L9 |
| 1352 | GVYYHKNNKSW | L16 | FYVLGLAAIM | L71; L72 | FGAISSVL | L61; L59; L28; |
| | | | | | | L25 |
| 1353 | GWEIVKFI | L48 | FYVYANGGKGF | L8; L9 | FGDSVEEV | L63; L68; L62; |
| | | | | | | L67 |
| 1354 | GWLIVGVALL | L8 | FYVYSRVKNL | L71; L72 | FHLDGEVIT | L35; L34; L28; |
| | | | | | | L44 |
| 1355 | GYKAIDGGVTR | L15 | FYYLGTGP | L71; L72 | FIASFRLF | L58; L59; L74; |
| | | | | | | L60 |
| 1356 | GYKKPASREL | L13 | FYYLGTGPE | L72; L71 | FISAARQGF | L58; L12; L60; |
| | | | | | | L16 |
| 1357 | GYLNSTNV | L71 | FYYSDSPC | L71; L72 | FKWDLTAF | L59; L27; L66; |
| | | | | | | L71 |
| 1358 | GYLQPRTFLLK | L6 | FYYVWKSYVH | L71; L72 | FLARGIVE | L74; L59; L26; |
| | | | | | | L23 |
| 1359 | GYLQPRTFLLKY | L12 | GAALQIPF | L74; L59 | FLCWHTNCY | L43; L59; L26; L1 |
| 1360 | GYLTSSSK | L71 | GAENSVAY | L18; L27 | FLEYHDVRV | L2; L63; L5; L4 |
| 1361 | GYREGYLNS | L13 | GALLTKSSEY | L27; L14 | FLIVAAIVFI | L2; L3; L4; L5 |
| 1362 | GYRVTKNSK | L13 | GCFVDDIVK | L7; L13 | FLMSFTVLC | L2; L4; L12; L5 |
| 1363 | GYVMHANYI | L9 | GDDTVIEV | L33; L37 | FLNKVVSTT | L3; L2; L4; L43 |
| 1364 | HAASGNLLLD | L49 | GDYGDAVVYR | L15; L19 | FLPGVYSVIY | L12; L66; L26; |
| | | | | | | L43 |
| 1365 | HAAVDALCE | L49 | GEAANFCA | L38; L46 | FLPGVYSVIYL | L2; L5; L57; L74 |
| 1366 | HADQLTPTWR | L63 | GEAANFCALIL | L36; L41 | FLQSINFVR | L21; L17; L15; |
| | | | | | | L19 |
| 1367 | HADQLTPTWRV | L18 | GEAVKTQFNY | L40; L41 | FLTENLLLYI | L3; L2; L4; L5 |
| | Y | | | | | |
| 1368 | HAFHTPAFD | L49 | GECPNFVFPL | L37; L36 | FLVQAGNVQL | L2; L4; L5; L74 |
| 1369 | HAFLCLFLLP | L50 | GEDIQLLKSAY | L41; L40 | FLYLYALV | L62; L2; L3; L47 |
| 1370 | HANEYRLYLD | L49 | GEQKSILSPLY | L40; L41 | FMIDVQQW | L49; L69; L54; L3 |
| 1371 | HANLDSCKR | L17 | GERSGARSK | L13; L45 | FPLKLRGTAV | L50; L51; L22; |
| | | | | | | L32 |
| 1372 | HDELTGHM | L33 | GETFVTHSK | L37; L38 | FPREGVFVSN | L50; L51; L31; |
| | | | | | | L22 |
| 1373 | HDELTGHMLDM | L18 | GETSWQTGD | L46; L37 | FRNARNGVL | L25; L66; L64; |
| | Y | | | | | L35 |
| 1374 | HDFFKFRI | L33 | GEVFNATRFA | L46; L38 | FSASTSAFV | L58; L70; L20; |
| | | | | | | L73 |
| 1375 | HDFFKFRID | L65 | GEYTFEKG | L45; L29 | FSHSQLGGL | L58; L60; L57; |
| | | | | | | L61 |
| 1376 | HDIGNPKAI | L37 | GFAAYSRY | L12; L14 | FSKLINII | L70; L48; L55; |
| | | | | | | L47 |
| 1377 | HDLYCQVH | L33 | GFAAYSRYR | L15; L17 | FSKLINIII | L70; L69; L55; |
| | | | | | | L68 |
| 1378 | HEFCSQHT | L29 | GFCDLKGKY | L12; L14 | FSNYLKRRV | L73; L64; L70; |
| | | | | | | L69 |
| 1379 | HEIAWYTE | L29 | GFNEKTHV | L62; L48 | FSQRGGSY | L1; L14; L59; L18 |
| 1380 | HEVLLAPLLS | L41 | GGAKLKAL | L23; L44 | FSSNVANY | L58; L1; L70; L59 |
| 1381 | HFDEGNCD | L62 | GGDAALAL | L68; L63 | FSTGVNLVA | L50; L61; L69; |
| | | | | | | L73 |
| 1382 | HFDEGNCDT | L62 | GGHSYGADL | L57; L44 | FSTGVNLVAV | L20; L73; L58; |
| | | | | | | L50 |
| 1383 | HFISNSWLMW | L8 | GGLHLLIGL | L44; L4 | FTEQPIDLV | L1; L20; L18; L63 |
| 1384 | HFYSKWYI | L48 | GGRFVLAL | L48; L23 | FTISVTTEIL | L60; L67; L61; |
| | | | | | | L20 |
| 1385 | HGFELTSMK | L13 | GHFDGQQG | L28; L35 | FTIYSLLLC | L73; L1; L12; L58 |
| 1386 | HGGGVAGAL | L44 | GHFDGQQGEV | L28; L35 | FTQSRNLQEF | L58; L10; L59; |
| | | | | | | L60 |
| 1387 | HGHVMVELV | L48 | GHLRIAGHHL | L34; L44 | FVCDNIKF | L58; L74; L68; |
| | | | | | | L63 |
| 1388 | HGKQVVSD | L23 | GHSYGADL | L28; L35 | FVENPDILR | L19; L17; L21; |
| | | | | | | L15 |
| 1389 | HGLNLEEAA | L50 | GHVMVELVA | L28; L35 | FVFPLNSIIK | L6; L7; L13; L19 |
| 1390 | HGLNLEEAARY | L14 | GIGVTQNVLY | L12; L18 | FVKHKHAF | L23; L59; L58; |
| | | | | | | L43 |
| 1391 | HGVVFLHV | L48 | GINASVVNIQK | L7; L6 | FVLAAVYRI | L4; L47; L5; L20 |
| 1392 | HHMELPTG | L28 | GISQYSLRL | L24; L73 | FVLWAHGFEL | L60; L61; L67; |
| | | | | | | L59 |
| 1393 | HHSIGFDYVY | L34 | GKFCLEASF | L27; L44 | FVNEFYAYLR | L15; L21; L17; |
| | | | | | | L19 |
| 1394 | HIDAYKTFP | L63 | GKQGNFKNL | L44; L27 | FVNLKQLPF | L59; L12; L58; |
| | | | | | | L60 |
| 1395 | HIDHPNPKG | L63 | GKYVQIPTT | L27; L44 | FVRIQPGQTF | L26; L43; L10; |
| | | | | | | L58 |
| 1396 | HIDHPNPKGF | L63 | GLALYYPSA | L2; L3 | FVSGNCDVV | L58; L67; L20; |
| | | | | | | L69 |
| 1397 | HISRQRLTK | L6 | GLDSLDTYPSL | L2; L5 | FVVPGLPGTI | L20; L10; L67; L3 |
| 1398 | HITSKETL | L23 | GLFCLLNR | L15; L21 | FWNCNVDRY | L58; L12; L49; |
| | | | | | | L14 |
| 1399 | HKHAFLCLFL | L44 | GLIAIVMVT | L3; L2 | FYDFAVSK | L62; L71; L68; |
| | | | | | | L72 |
| 1400 | HKLVLSVNP | L44 | GLKTILRK | L13; L6 | FYEDFLEYH | L62; L72; L71; |
| | | | | | | L12 |
| 1401 | HKPIVWHV | L48 | GLMWLSYFI | L24; L2 | FYGGWHNM | L72; L66; L71; |
| | | | | | | L62 |
| 1402 | HKPIVWHVN | L44 | GLNGYTVEEA | L3; L2 | FYILPSII | L62; L71; L72; |
| | | | | | | L47 |
| 1403 | HLIPLMYK | L6 | GLNLEEAAR | L21; L15 | FYWFFSNY | L72; L71; L66; |
| | | | | | | L12 |
| 1404 | HLIPLMYKG | L24 | GLQPSVGPK | L6; L7 | GANKDGIIW | L54; L49; L52; |
| | | | | | | L53 |
| 1405 | HLKDGTCGLV | L3 | GLVEVEKGVL | L44; L2 | GEAVKTQF | L33; L29; L37; |
| | | | | | | L45 |
| 1406 | HLKDGTCGLVEV | L3 | GLYKNTCV | L24; L2 | GECPNFVF | L29; L37; L33; |
| | | | | | | L36 |
| 1407 | HLLIGLAKRF | L12 | GLYPTLNIS | L24; L3 | GEDIQLLKS | L45; L46; L37; |
| | | | | | | L41 |
| 1408 | HLMGWDYPK | L6 | GNKGAGGHSY | L14; L26 | GEDIQLLKSA | L46; L38; L42; |
| | | | | | | L37 |
| 1409 | HLRIAGHHL | L26 | GNLQSNHDLY | L12; L14 | GEFKLASHMY | L40; L29; L41; |
| | | | | | | L27 |
| 1410 | HLSVDTKFK | L6 | GNYNYLYRL | L4; L24 | GEIPVAYRKVL | L36; L41; L40; |
| | | | | | | L37 |
| 1411 | HLSWEVGKP | L24 | GNYQCGHYK | L7; L6 | GGDAALALL | L63; L62; L68; |
| | | | | | | L34 |
| 1412 | HLVDFQVTIA | L3 | GPKKSTNLV | L51; L39 | GGFNFSQIL | L44; L64; L28; |
| | | | | | | L69 |
| 1413 | HLYFDKAGQK | L6 | GPLVRKIFV | L23; L47 | GHFDGQQGE | L28; L34; L35; |
| | | | | | | L24 |
| 1414 | HLYLQYIR | L21 | GPPGTGKSH | L39; L22 | GHSMQNCVL | L35; L28; L34; |
| | | | | | | L44 |
| 1415 | HMELPTGV | L48 | GQGLNGYTV | L24; L48 | GIATVREVL | L44; L16; L28; |
| | | | | | | L67 |
| 1416 | HMLDMYSV | L48 | GQGVPINTN | L24; L26 | GIDLDEWSM | L68; L63; L28; |
| | | | | | | L18 |
| 1417 | HMLDMYSVM | L72 | GQINDMIL | L28; L44 | GIVFMCVEY | L26; L43; L12; |
| | | | | | | L27 |
| 1418 | HMLDMYSVML | L44 | GQIVTCAKEI | L24; L48 | GIYQTSNFR | L21; L7; L6; L19 |
| 1419 | HNLAKHCL | L23 | GQPITNCV | L48; L24 | GKYEQYIKW | L27; L44; L49; |
| | | | | | | L10 |
| 1420 | HNSHEGKTFY | L12 | GQSKRVDF | L27; L26 | GLAAIMQL | L3; L74; L2; L4 |
| 1421 | HPTQAPTH | L50 | GQSTQLGI | L48; L24 | GLAAVNSV | L3; L2; L4; L24 |
| 1422 | HQECSLQSC | L24 | GQSTQLGIEF | L26; L27 | GLCVDIPGI | L4; L2; L24; L3 |
| 1423 | HQSDIEVTGD | L44 | GRIRSVYPV | L30; L35 | GLEAPFLYLY | L18; L12; L1; L14 |
| 1424 | HRFYRLAN | L25 | GRRGPEQTQ | L65; L30 | GLFKDCSKV | L24; L3; L2; L4 |
| 1425 | HRFYRLANE | L25 | GRSGETLGVL | L30; L35 | GLIAIVMV | L24; L2; L3; L4 |
| 1426 | HRLYECLY | L65 | GSALLEDEF | L53; L52 | GLPNNTASW | L57; L10; L16; L5 |
| 1427 | HRLYECLYR | L30 | GSDNVTDEN | L1; L18 | GLYPTLNI | L24; L48; L3; L2 |
| 1428 | HRLYECLYRNR | L30 | GSGVPVVDSY | L14; L18 | GPEHSLAEY | L49; L31; L18; |
| | | | | | | L12 |
| 1429 | HSCNVNRFN | L55 | GSSGVVNPV | L20; L24 | GPKKSTNL | L56; L39; L23; |
| | | | | | | L22 |
| 1430 | HSMQNCVL | L55 | GSTPCNGVEGF | L52; L54 | GQQFGPTY | L26; L27; L48; |
| | | | | | | L14 |
| 1431 | HSSSSDNIAL | L44 | GSVAYESL | L67; L55 | GQQFGPTYL | L44; L28; L24; |
| | | | | | | L34 |
| 1432 | HSTQDLFLP | L53 | GSVAYESLR | L19; L7 | GQTGKIADY | L26; L14; L27; |
| | | | | | | L12 |
| 1433 | HSTQDLFLPF | L52 | GTAVLRQWL | L53; L16 | GRSGETLGV | L30; L24; L35; |
| | | | | | | L34 |
| 1434 | HSTQDLFLPFF | L52 | GTDPYEDFQ | L1; L18 | GSFCTQLNR | L7; L15; L19; L21 |
| 1435 | HSWNADLYKL | L53 | GTDPYEDFQEN | L52; L54 | GSLPINVI | L48; L55; L47; |
| | | | W | | | L53 |
| 1436 | HTGTGQAITV | L20 | GTDTTITVN | L18; L1 | GTDLEGNFY | L1; L18; L14; L12 |
| 1437 | HTKKWKYPQV | L13 | GTEDDYQGK | L13; L7 | GTGTIYTEL | L53; L10; L67; |
| | | | | | | L65 |
| 1438 | HTPINLVRD | L11 | GTGTSTDVVY | L18; L52 | GTITVEELKK | L7; L13; L6; L19 |
| 1439 | HTPINLVRDL | L11 | GTILTRPL | L55; L60 | GTLEPEYF | L52; L55; L54; |
| | | | | | | L53 |
| 1440 | HTTCCSLSHRF | L54 | GTLIVNSVLL | L53; L13 | GTLIVNSVL | L53; L44; L55; |
| | | | | | | L52 |
| 1441 | HTTDPSFLG | L20 | GTLSYEQFKK | L7; L6 | GTTLPKGFY | L14; L1; L12; L18 |
| 1442 | HTVLQAVGA | L50 | GTNLPLQLGF | L12; L52 | GVAMPNLYK | L6; L7; L13; L19 |
| 1443 | HTVMPLSA | L50 | GTPVCINGL | L57; L10 | GVCVSTSGR | L19; L15; L21; L7 |
| 1444 | HTVMPLSAP | L10 | GTSSGDATTAY | L18; L14 | GVKHVYQLR | L15; L21; L19; |
| | | | | | | L17 |
| 1445 | HVDILGPLS | L1 | GTTSPISEH | L7; L10 | GVPFVVSTGY | L11; L12; L26; |
| | | | | | | L14 |
| 1446 | HVDTDLTK | L18 | GTTTLNGLW | L52; L54 | GVVDYGARFY | L12; L14; L10; |
| | | | | | | L11 |
| 1447 | HVICTSEDM | L11 | GTVSWNLR | L19; L15 | GVYFASTEK | L7; L6; L13; L19 |
| 1448 | HVISTSHKLV | L20 | GVAGALNKA | L51; L3 | GWHNMLKTV | L24; L3; L48; L45 |
| 1449 | HVMSKHTD | L23 | GVALLAVF | L26; L59 | GYLPQNAVV | L72; L8; L71; L9 |
| 1450 | HVNNATNKA | L51 | GVDAVNLLT | L18; L1 | HADFDTWFS | L68; L49; L18; |
| | | | | | | L63 |
| 1451 | HVNNATNKATY | L14 | GVEGFNCY | L18; L1 | HCANFNVL | L35; L28; L25; |
| | | | | | | L44 |
| 1452 | HVRAWIGF | L26 | GVFVSNGTHW | L10; L52 | HDVRVVLDF | L33; L49; L65; |
| | | | | | | L29 |
| 1453 | HVSGTNGTKRF | L11 | GVFVSNGTHWF | L16; L26 | HDYQIGGY | L29; L33; L14; |
| | | | | | | L11 |
| 1454 | HVTFFIYNKI | L20 | GVGGKPCIK | L6; L7 | HEETIYNL | L37; L33; L29; |
| | | | | | | L36 |
| 1455 | HVTYVPAQE | L20 | GVKDCVVLH | L13; L7 | HEFCSQHTML | L37; L36; L45; |
| | | | | | | L25 |
| 1456 | HVTYVPAQEK | L19 | GVLITEGSVK | L6; L7 | HEGKTFYV | L33; L45; L37; |
| | | | | | | L29 |
| 1457 | HVYQLRARSV | L24 | GVLPQLEQPY | L12; L14 | HEHEIAWY | L29; L37; L33; |
| | | | | | | L45 |
| 1458 | HWFVTQRN | L48 | GVLTAVVIPTK | L7; L6 | HFISNSWLM | L8; L72; L12; L71 |
| 1459 | HWFVTQRNFY | L12 | GVLTAVVIPTKK | L7; L6 | HFRELGVVH | L72; L71; L13; |
| | | | | | | L43 |
| 1460 | HYQECVRGT | L72 | GVTFQSAVKR | L19; L7 | HGFELTSMKY | L14; L12; L11; |
| | | | | | | L29 |
| 1461 | HYVYIGDPA | L50 | GVTLIGEAV | L20; L24 | HHANEYRL | L34; L35; L28; |
| | | | | | | L44 |
| 1462 | IAARDLICA | L50 | GVTQNVLY | L12; L18 | HISRQRLTKY | L11; L14; L18; |
| | | | | | | L10 |
| 1463 | IAARDLICAQKF | L52 | GVVTTVMFLAR | L7; L19 | HKDKSAQCF | L34; L49; L27; |
| | | | | | | L63 |
| 1464 | IADKYVRN | L63 | GVYYPDKV | L48; L24 | HKPPISFPL | L66; L44; L57; |
| | | | | | | L64 |
| 1465 | LADYNYKLP | L63 | GWTAGAAAYY | L12; L14 | HLIPLMYKGL | L3; L11; L10; L5 |
| 1466 | IAFGGCVFS | L69 | GYINVFAFP | L9; L12 | HNIALIWNV | L20; L24; L73; L4 |
| 1467 | IAGLIAIVMV | L50 | GYKKPASR | L15; L17 | HNSHEGKTF | L49; L34; L32; |
| | | | | | | L53 |
| 1468 | IAIILASFSA | L50 | GYLKLTDNVY | L12; L14 | HPNCVNCL | L32; L56; L39; |
| | | | | | | L23 |
| 1469 | IAKKPTET | L43 | GYLNSTNVTI | L8; L9 | HPNQEYADVF | L32; L31; L49; |
| | | | | | | L39 |
| 1470 | IALIWNVKDF | L52 | GYLPQNAVVK | L13; L7 | HSIGFDYV | L73; L20; L48; |
| | | | | | | L67 |
| 1471 | IALKGGKI | L47 | GYLPQNAVVKI | L9; L8 | HSWNADLY | L18; L1; L14; L54 |
| 1472 | IALKGGKIVNNW | L52 | GYLQPRTFLL | L9; L8 | HTSPDVDLG | L52; L53; L18; |
| | | | | | | L20 |
| 1473 | IAMACLVGLMW | L52 | GYVDTPNNTDF | L8; L9 | HTTDPSFLGRY | L14; L11; L18; |
| | | | | | | L10 |
| 1474 | IANQFNSA | L50 | GYVMHANY | L71; L72 | HVDTDLTKP | L63; L18; L49; |
| | | | | | | L11 |
| 1475 | IANYAKPF | L59 | GYVMHANYIF | L8; L9 | IAANTVIWD | L69; L70; L74; |
| | | | | | | L49 |
| 1476 | IAQFAPSA | L50 | GYYRRATR | L15; L17 | IAANTVIWDY | L31; L12; L58; |
| | | | | | | L14 |
| 1477 | IASEFSSLPSY | L31 | GYYRRATRR | L15; L17 | IAPGQTGKI | L47; L49; L63; |
| | | | | | | L57 |
| 1478 | IASTDTCF | L54 | HAIHVSGTN | L32; L49 | ICHDGKAHF | L43; L49; L8; L9 |
| 1479 | IAYIICIS | L47 | HANYIFWR | L17; L15 | ICYTPSKL | L74; L48; L47; |
| | | | | | | L25 |
| 1480 | IAYTMSLG | L47 | HANYIFWRN | L49; L32 | IDHPNPKGF | L33; L43; L29; |
| | | | | | | L52 |
| 1481 | IAYTMSLGA | L50 | HDELTGHML | L37; L34 | IDYDCVSF | L33; L43; L71; |
| | | | | | | L29 |
| 1482 | ICHDGKAH | L43 | HDVRVVLD | L65; L33 | IEFLKRGD | L33; L45; L46; |
| | | | | | | L65 |
| 1483 | ICISTKHFYWF | L8 | HECFVKRVDW | L41; L40 | IFFASFYYV | L47; L62; L72; |
| | | | | | | L71 |
| 1484 | ICLLQFAYA | L50 | HEGKTFYVLP | L38; L42 | IFGADPIHSL | L72; L9; L8; L71 |
| 1485 | IDAFKLNI | L33 | HFAWWTAFV | L62; L20 | IFLIVAAI | L71; L48; L72; |
| | | | | | | L47 |
| 1486 | IDCSARHI | L55 | HFIETISLAGSY | L14; L12 | IFLIVAAIV | L71; L72; L47; |
| | | | | | | L62 |
| 1487 | IDFYLCFL | L33 | HFLPRVFSAV | L71; L72 | IGFLFLTW | L48; L47; L54; |
| | | | | | | L52 |
| 1488 | IDFYLCFLAF | L33 | HFRELGVV | L48; L62 | IGINITRF | L29; L54; L27; |
| | | | | | | L26 |
| 1489 | IDGGVTRD | L65 | HFYWFFSNYL | L71; L72 | IIIGGAKLK | L7; L6; L19; L13 |
| 1490 | IDLDEWSM | L33 | HGLAAVNSV | L48; L47 | IINNTVYTK | L7; L6; L19; L21 |
| 1491 | IDLLLDDF | L33 | HGLNLEEA | L25; L50 | IKLIFLWLL | L44; L4; L64; L8 |
| 1492 | IDLVPNQPY | L12 | HGVVFLHVTY | L29; L26 | ILANTCTER | L21; L19; L15; L6 |
| 1493 | IDVQQWGF | L33 | HKHAFLCLF | L27; L34 | ILLNKHIDAY | L12; L14; L6; L16 |
| 1494 | IDYKHYTP | L33 | HLDGEVIT | L62; L63 | ILLNVPLHG | L4; L12; L6; L2 |
| 1495 | IDYKHYTPSF | L33 | HLLIGLAK | L6; L23 | ILSDDAVVCF | L26; L9; L16; L49 |
| 1496 | IEELFYSYAT | L38 | HMVVKAAL | L23; L25 | INDMILSL | L68; L63; L62; |
| | | | | | | L67 |
| 1497 | IEFLKRGDK | L46 | HMVVKAALL | L23; L62 | INDMILSLL | L68; L62; L63; L5 |
| 1498 | IEFLKRGDKSV | L45 | HNESGLKTI | L49; L34 | INFVRIIMR | L17; L21; L15; |
| | | | | | | L19 |
| 1499 | IELKFNPP | L46 | HNMLKTVY | L29; L65 | IPIQASLPF | L22; L31; L49; |
| | | | | | | L50 |
| 1500 | IELSLIDFYLC | L40 | HPDSATLVS | L32; L49 | IPKEEVKPF | L39; L49; L31; |
| | | | | | | L22 |
| 1501 | IELSLIDFYLCF | L41 | HPDSATLVSD | L49; L32 | IQPGQTFSV | L5; L24; L48; L57 |
| 1502 | IERFVSLA | L46 | HPLADNKFA | L51; L50 | IRKSNHNFL | L25; L66; L64; |
| | | | | | | L30 |
| 1503 | IETISLAGS | L46 | HPNCVNCLD | L32; L49 | ISAGFSLW | L54; L52; L53; |
| | | | | | | L18 |
| 1504 | IEVNSFSGYL | L36 | HPTQAPTHLSV | L50; L51 | ISDEFSSN | L68; L1; L63; L18 |
| 1505 | IEVNSFSGYLKL | L36 | HSCNVNRF | L53; L54 | ISDEVARD | L63; L1; L68; L54 |
| 1506 | IEVQGYKS | L29 | HSDKFTDGVCL | L63; L68 | ISDEVARDLSL | L68; L63; L67; L1 |
| 1507 | IEVQGYKSVN | L46 | HSMQNCVLKL | L73; L53 | ISMWALII | L55; L48; L47; |
| | | | | | | L73 |
| 1508 | IEVTGDSC | L29 | HSQLGGLHLL | L53; L18 | ISVTTEIL | L67; L74; L60; |
| | | | | | | L54 |
| 1509 | IEYPIIGDE | L46 | HSSRLSFKEL | L53; L73 | ITEEVGHTDL | L63; L68; L1; L60 |
| 1510 | IEYPIIGDELKI | L45 | HSTQDLFL | L73; L74 | ITHDVSSAI | L53; L61; L70; |
| | | | | | | L68 |
| 1511 | IFFASFYYVW | L9 | HSWNADLYK | L13; L7 | ITHDVSSAINR | L19; L21; L15; |
| | | | | | | L17 |
| 1512 | IFLEGETLP | L8 | HSYFTSDYYQL | L1; L18 | ITSKETLY | L1; L18; L54; L53 |
| | | | Y | | | |
| 1513 | IFLWLLWPV | L71 | HTANKWDLI | L20; L53 | ITTYPGQGL | L60; L61; L53; |
| | | | | | | L20 |
| 1514 | IFWRNTNPI | L8 | HTDFSSEIIGY | L18; L1 | IVDEPEEH | L63; L68; L62; |
| | | | | | | L67 |
| 1515 | IFYLITPVHV | L62 | HTFSNYQHE | L10; L11 | IVFDGKSK | L13; L19; L7; L6 |
| 1516 | IGAEHVNNSY | L14 | HTQVVDMSM | L32; L53 | IVFITLCF | L26; L59; L54; |
| | | | | | | L52 |
| 1517 | IGEAVKTQF | L63 | HVASCDAIM | L32; L11 | IVGVALLAV | L73; L4; L51; L5 |
| 1518 | IGEAVKTQFNYY | L18 | HVDILGPLSA | L18; L50 | IVKTDGTLM | L58; L53; L11; |
| | | | | | | L52 |
| 1519 | IGEQKSIL | L63 | HVGEIPVA | L50; L51 | IYQTSNFRV | L8; L9; L71; L72 |
| 1520 | IGGAKLKAL | L23 | HVMVELVAEL | L20; L10 | KADETQALP | L63; L54; L18; |
| | | | | | | L68 |
| 1521 | IGGYTEKW | L54 | HVVDGCNSS | L10; L11 | KAPKEIIF | L63; L74; L54; |
| | | | | | | L53 |
| 1522 | IGKIQDSL | L23 | HVVDGCNSST | L11; L10 | KATNNAMQV | L70; L73; L69; |
| | | | | | | L58 |
| 1523 | IGPERTCC | L57 | HYTPSFKKG | L9; L8 | KAVFISPY | L70; L43; L59; |
| | | | | | | L54 |
| 1524 | IGPERTCCL | L57 | HYVYIGDPAQL | L8; L9 | KCTSVVLL | L56; L73; L55; |
| | | | | | | L74 |
| 1525 | IGVTQNVL | L25 | IADTTDAVR | L68; L63 | KCYGVSPTKL | L6; L8; L73; L30 |
| 1526 | IGYYRRATR | L15 | IADTTDAVRD | L63; L49 | KESPFELEDF | L36; L41; L33; |
| | | | | | | L40 |
| 1527 | IHFGAGSDK | L28 | IAEILLII | L47; L68 | KGEDIQLLK | L18; L13; L7; L6 |
| 1528 | IHFYSKWY | L27 | IAGLIAIV | L47; L70 | KGGKIVNNW | L54; L16; L52; |
| | | | | | | L53 |
| 1529 | IHFYSKWYI | L34 | IALKGGKIV | L47; L70 | KGGRFVLAL | L73; L65; L44; |
| | | | | | | L67 |
| 1530 | IHSLRVCV | L28 | ICISTKHFYW | L52; L54 | KHTDFSSEI | L35; L34; L28; L8 |
| 1531 | IHTIDGSSG | L28 | ICYTPSKLI | L47; L45 | KIAEIPKEEV | L2; L3; L20; L5 |
| 1532 | IHTIDGSSGV | L28 | IDFLELAM | L33; L29 | KIITLKKRW | L54; L52; L16; |
| | | | | | | L14 |
| 1533 | IHTIDGSSGVV | L28 | IDLQELGKY | L14; L12 | KILGLPTQTV | L16; L24; L2; L4 |
| 1534 | IHVSGTNGT | L28 | IDSYFVVKR | L19; L21 | KKLDGFMGR | L21; L15; L27; |
| | | | | | | L17 |
| 1535 | IICISTKHF | L26 | IDYTEISF | L33; L29 | KKPASREL | L66; L57; L27; |
| | | | | | | L64 |
| 1536 | IICISTKHFY | L18 | IEDLLFNKVTL | L36; L41 | KLDGVVCTEI | L5; L68; L63; L2 |
| 1537 | IIGDELKI | L24 | IELSLIDF | L33; L29 | KLFAAETLK | L6; L21; L7; L13 |
| 1538 | IIKLIFLW | L52 | IERYKLEGY | L29; L26 | KLIEYTDF | L26; L43; L27; |
| | | | | | | L54 |
| 1539 | IIKSQDLSV | L3 | IETISLAGSY | L40; L41 | KLIFLWLL | L74; L4; L55; L2 |
| 1540 | IIKSQDLSVV | L3 | IEYTDFATS | L46; L45 | KLKTLVATA | L3; L51; L13; L2 |
| 1541 | IIKTIQPR | L21 | IFLWLLWPVTL | L9; L8 | KLLEQWNLVI | L16; L2; L4; L8 |
| 1542 | IILKPANNSL | L16 | IFYLITPVHVM | L71; L72 | KLNDLCFTNVY | L14; L16; L18; |
| | | | | | | L26 |
| 1543 | IIMRLWLCWK | L6 | IGFLFLTWI | L47; L48 | KLTDNVYIK | L6; L13; L16; L7 |
| 1544 | IIMRTFKV | L48 | IGIVNNTV | L48; L47 | KNVSLDNVL | L44; L73; L67; |
| | | | | | | L36 |
| 1545 | IIPARARV | L5 | IGNYTVSCL | L57; L65 | KPANNSLKI | L24; L47; L49; |
| | | | | | | L51 |
| 1546 | IIPARARVE | L57 | IHADQLTP | L35; L28 | KPPPGDQF | L39; L56; L22; |
| | | | | | | L66 |
| 1547 | IIRENNRV | L48 | IIIWFLLLS | L73; L6 | KPRPPLNRNY | L52; L13; L14; |
| | | | | | | L22 |
| 1548 | IISDMYDPK | L6 | IILFLALI | L48; L47 | KRFDNPVLP | L64; L30; L65; |
| | | | | | | L66 |
| 1549 | IISDMYDPKTK | L6 | IILFLALITL | L4; L67 | KRSFIEDLL | L64; L34; L66; |
| | | | | | | L30 |
| 1550 | IISNEKQEI | L24 | IIMRLWLCW | L54; L52 | KSAFVNLKQ | L54; L73; L18; |
| | | | | | | L53 |
| 1551 | IITTDNTF | L54 | IIPLTTAAK | L57; L6 | KSATCITRC | L54; L53; L18; |
| | | | | | | L52 |
| 1552 | IITTVAAF | L59 | IIPLTTAAKL | L57; L5 | KSEDAQGM | L55; L18; L54; |
| | | | | | | L63 |
| 1553 | IKESVQTF | L27 | IIQFPNTY | L26; L18 | KSFTVEKGIY | L54; L14; L18; |
| | | | | | | L52 |
| 1554 | IKESVQTFF | L27 | IISVTSNY | L18; L26 | KSHFAIGLAL | L57; L61; L73; |
| | | | | | | L60 |
| 1555 | IKIQEGVVD | L27 | IKFADDLNQL | L27; L44 | KSNIIRGWIF | L53; L54; L55; |
| | | | | | | L52 |
| 1556 | IKIQEGVVDY | L27 | IKGTHHWLL | L66; L44 | KSQDLSVVSK | L13; L7; L6; L15 |
| 1557 | IKLDDKDPNF | L9 | IKNFKSVLYY | L27; L12 | KSREETGL | L53; L55; L65; |
| | | | | | | L54 |
| 1558 | IKLIFLWLLW | L52 | IKWPWYIWLGF | L27; L8 | KTCGQQQTT | L54; L53; L55; |
| | | | | | | L16 |
| 1559 | IKNADIVEE | L27 | ILANTCTERL | L4; L2 | KTCGQQQTTL | L53; L16; L55; |
| | | | | | | L54 |
| 1560 | IKNLSKSL | L27 | ILDGISQYS | L5; L1 | KTFPPTEPKK | L13; L7; L6; L52 |
| 1561 | IKPVTYKL | L66 | ILFTRFFY | L12; L18 | KTFYVLPND | L13; L53; L54; |
| | | | | | | L52 |
| 1562 | IKRSDARTA | L27 | ILLNVPLHGT | L2; L4 | KTGDLQPL | L55; L73; L54; |
| | | | | | | L53 |
| 1563 | IKTIQPRV | L48 | ILNNLGVDI | L24; L4 | KTIGPDMF | L54; L52; L55; |
| | | | | | | L53 |
| 1564 | IKWADNNCY | L27 | ILPDPSKPSK | L6; L5 | KTLLSLREV | L73; L13; L52; |
| | | | | | | L55 |
| 1565 | ILASFSAST | L43 | ILPSIISNE | L5; L57 | KTLQPVSELL | L53; L16; L54 |
| | | | | | | L52 |
| 1566 | ILCIMSDRDLY | L18 | ILPSIISNEK | L6; L57 | KTTLPVNVAF | L53; L52; L16; |
| | | | | | | L54 |
| 1567 | ILFLALITLA | L3 | ILPVSMTKT | L57; L5 | KVFRSSVLH | L6; L7; L21; L54 |
| 1568 | ILFTRFFYVL | L4 | ILRGHLRIA | L3; L51 | KVPTDNYITTY | L18; L11; L16; |
| | | | | | | L14 |
| 1569 | ILGTVSWNLR | L21 | IMLVYCFLGY | L12; L6 | KVTIDYTEI | L53; L55; L54; |
| | | | | | | L24 |
| 1570 | ILHCANFNVL | L57 | IMQLFFSY | L18; L26 | KVTLVFLF | L54; L55; L52; |
| | | | | | | L33 |
| 1571 | ILKPANNSLK | L6 | IMQLFFSYF | L9; L26 | KWDLIISDM | L62; L63; L68; |
| | | | | | | L18 |
| 1572 | ILLAKDTTEA | L2 | INASVVNIQK | L19; L7 | KWDLTAFGL | L62; L68; L63; |
| | | | | | | L36 |
| 1573 | ILLIIMRTFK | L6 | INLIIKNL | L48; L25 | KWGKARLYY | L12; L18; L14; |
| | | | | | | L16 |
| 1574 | ILLLDQAL | L23 | INRPQIGVV | L3; L73 | KYLVQQESPF | L8; L9; L72; L71 |
| 1575 | ILLNKHIDA | L2 | IPARARVECF | L49; L31 | KYNENGTI | L9; L71; L8; L72 |
| 1576 | ILMTARTV | L48 | IPDYNTYKN | L49; L32 | KYNENGTIT | L8; L72; L9; L71 |
| 1577 | ILPSIISN | L57 | IPFAMQMA | L50; L51 | LAAVNSVPW | L49; L52; L61; |
| | | | | | | L31 |
| 1578 | ILPVSMTK | L57 | IPFAMQMAYRF | L31; L49 | LAFVVFLLVTL | L31; L69; L59; |
| | | | | | | L47 |
| 1579 | ILRGHLRI | L48 | IPMDSTVKNY | L31; L49 | LALYNKYKY | L12; L31; L52; |
| | | | | | | L14 |
| 1580 | ILSDDAVV | L2 | IPVAYRKV | L47; L51 | LATNNLVV | L47; L70; L50; |
| | | | | | | L69 |
| 1581 | ILSLLSKGR | L21 | IPYNSVTSSI | L47; L22 | LAVPYNMRVI | L47; L69; L70; |
| | | | | | | L55 |
| 1582 | ILTALRLCAY | L12 | IQEGVVDY | L26; L27 | LAWLYAAV | L47; L50; L59; |
| | | | | | | L67 |
| 1583 | IMASLVLARK | L6 | IQKDVLEC | L48; L26 | LAYCNKTV | L47; L70; L69; |
| | | | | | | L48 |
| 1584 | IMLIIFWF | L33 | IQLLKSAY | L27; L26 | LAYYFMRFR | L17; L69; L59; |
| | | | | | | L47 |
| 1585 | IMLIIFWFSL | L4 | IQPGQTFSVL | L57; L26 | LDYIINLI | L47; L48; L33; |
| | | | | | | L25 |
| 1586 | IMMNVAKY | L14 | IQPIGALDI | L24; L48 | LEFGATSAA | L46; L38; L42; |
| | | | | | | L37 |
| 1587 | IMRLWLCW | L48 | IRASANLAA | L35; L46 | LEFGATSAAL | L36; L41; L37; |
| | | | | | | L40 |
| 1588 | IMRLWLCWK | L13 | IRENNRVVI | L34; L35 | LEGSVRVV | L45; L37; L38; |
| | | | | | | L48 |
| 1589 | IMRTFKVSI | L48 | IRQEEVQELY | L30; L14 | LETAQNSV | L45; L46; L37; |
| | | | | | | L38 |
| 1590 | IMRTFKVSIW | L52 | IRQLLFVV | L25; L35 | LETIQITI | L45; L48; L37; |
| | | | | | | L29 |
| 1591 | IMTRCLAV | L48 | IRQLLFVVEV | L64; L30 | LFDRYFKY | L1; L62; L12; L18 |
| 1592 | INFVRIIMRLW | L52 | ISAMVRMYI | L55; L73 | LFENKTTL | L62; L71; L72; |
| | | | | | | L23 |
| 1593 | INGDRWFLNR | L17 | ISDEFSSNVA | L46; L68 | LFFFLYENAF | L72; L71; L12; L9 |
| 1594 | INGLMLLEI | L73 | ISDEFSSNVANY | L1; L18 | LFLPFFSNV | L47; L8; L71; L72 |
| 1595 | INIIIWFL | L73 | ISEHDYQIGGY | L1; L18 | LGFSTGVNL | L44; L56; L61; |
| | | | | | | L49 |
| 1596 | INLHTQVV | L48 | ISGINASVV | L73; L48 | LIDLQELGKY | L1; L18; L14; L12 |
| 1597 | INNTVYTKV | L73 | ISLAGSYKD | L54; L55 | LIIFWFSLEL | L60; L61; L73; |
| | | | | | | L59 |
| 1598 | INRPQIGV | L48 | ISMDNSPNLAW | L52; L54 | LITLATCEL | L60; L59; L61; |
| | | | | | | L67 |
| 1599 | IPGIPKDMTY | L31 | ISMMGFKMNY | L1; L18 | LKAPKEIIF | L27; L44; L66; |
| | | | | | | L69 |
| 1600 | IPIGAGICASY | L31 | ISNCVADY | L54; L18 | LKSPNFSKL | L44; L66; L64; |
| | | | | | | L27 |
| 1601 | IPKDMTYRRL | L39 | ISNSWLMW | L54; L73 | LLALHRSY | L26; L59; L1; L18 |
| 1602 | IPKDMTYRRLI | L39 | ISQYSLRL | L73; L60 | LLFLAFVVFL | L4; L2; L3; L5 |
| 1603 | IPKEEVKP | L51 | ISSVLNDIL | L67; L53 | LLFLMSFTV | L5; L2; L4; L3 |
| 1604 | IPKEEVKPFI | L47 | ISTKHFYW | L54; L52 | LLHKPIVWHV | L3; L5; L4; L2 |
| 1605 | IPMDSTVKNYF | L31 | ITDAQTGSS | L1; L18 | LLLDKRTTC | L4; L23; L16; L2 |
| 1606 | IPRRNVATLQA | L51 | ITDAQTGSSK | L18; L1 | LLSDLQDL | L4; L3; L57; L59 |
| 1607 | IPTITQMNLKY | L31 | ITDAQTGSSKC | L18; L1 | LLTILTSL | L23; L59; L74; |
| | | | | | | L25 |
| 1608 | IPYNSVTS | L47 | ITDVFYKEN | L1; L68 | LLTLQQIEL | L74; L68; L2; L56 |
| 1609 | IPYNSVTSSIV | L47 | ITDVFYKENSY | L1; L18 | LMCQPILLL | L5; L4; L61; L34 |
| 1610 | IPYNSVTSSIVI | L47 | ITEHSWNADLY | L1; L18 | LNDFNLVAM | L68; L62; L63; |
| | | | | | | L32 |
| 1611 | IQDSLSSTA | L28 | ITFDNLKTLL | L20; L53 | LNDLNETL | L68; L63; L62; |
| | | | | | | L67 |
| 1612 | IQFPNTYLE | L24 | ITGGIAIAM | L70; L58 | LNDLNETLV | L63; L62; L1; L68 |
| 1613 | IQITISSFK | L13 | ITGRLQSL | L53; L54 | LPDPSKPSK | L32; L22; L31; |
| | | | | | | L49 |
| 1614 | IQITISSFKW | L54 | ITILDGISQ | L53; L52 | LPETTADIVVF | L31; L39; L32; |
| | | | | | | L49 |
| 1615 | IQKEIDRL | L48 | ITLCFTLK | L6; L7 | LPFGWLIV | L47; L50; L51; |
| | | | | | | L39 |
| 1616 | IQRKYKGI | L48 | ITLCFTLKRK | L7; L6 | LPFGWLIVGVAL | L56; L31; L22; |
| | | | | | | L39 |
| 1617 | IQRKYKGIKI | L48 | ITQMNLKY | L18; L1 | LPGTILRTT | L51; L50; L32; |
| | | | | | | L56 |
| 1618 | IQTIVEVQ | L48 | ITREEAIRH | L53; L52 | LPIDKCSRII | L47; L32; L49; |
| | | | | | | L39 |
| 1619 | IQTTPGSGV | L28 | ITREEAIRHV | L13; L52 | LPKGIMMNV | L51; L47; L39; |
| | | | | | | L50 |
| 1620 | IQWMVMFTP | L24 | ITVNVLAW | L54; L52 | LPPLLTDEM | L31; L22; L47; |
| | | | | | | L32 |
| 1621 | IRAAEIRAS | L64 | IVAGGIVA | L51; L50 | LPVLQVRDV | L47; L50; L51; |
| | | | | | | L39 |
| 1622 | IRENNRVV | L64 | IVAGGIVAIV | L20; L3 | LQFTSLEI | L48; L24; L47; |
| | | | | | | L25 |
| 1623 | IRGGDGKMK | L30 | IVAIVVTCLAY | L12; L18 | LQKAAITIL | L44; L58; L26; |
| | | | | | | L69 |
| 1624 | IRHVRAWIGF | L30 | IVDEPEEHVQ | L63; L68 | LQSLENVAF | L26; L27; L31; |
| | | | | | | L34 |
| 1625 | IRQGTDYKH | L30 | IVDSVTVK | L63; L68 | LRGTAVMSL | L65; L66; L35; |
| | | | | | | L64 |
| 1626 | IRVGARKSA | L30 | IVEVQPQL | L63; L68 | LSDRELHLSW | L52; L1; L54; L18 |
| 1627 | ISAARQGFV | L73 | IVFITLCFTLK | L6; L7 | LSDRVVFV | L63; L68; L62; L1 |
| 1628 | ISAGFSLWVYK | L7 | IVFMCVEY | L26; L59 | LSGLDSLDTY | L18; L1; L52; L14 |
| 1629 | ISAMVRMYIF | L53 | IVNNATNVVIK | L7; L6 | LTILTSLL | L60; L1; L59; L61 |
| 1630 | ISDEFSSNVAN | L68 | IVVTCLAYYF | L12; L52 | LTILTSLLVL | L67; L60; L59; |
| | | | | | | L73 |
| 1631 | ISDEVARDLS | L1 | IWNVKDFMSL | L8; L9 | LTKPYIKW | L52; L54; L53; |
| | | | | | | L55 |
| 1632 | ISDYDYYR | L1 | IWVATEGAL | L28; L67 | LVFLGIITT | L50; L51; L69; |
| | | | | | | L13 |
| 1633 | ISFPLCAN | L59 | IYDEPTTTTS | L62; L9 | LVKNKCVNF | L58; L26; L43; |
| | | | | | | L52 |
| 1634 | ISFPLCANGQVF | L52 | IYDEPTTTTSV | L62; L5 | LVSTQEFRY | L12; L14; L18; L1 |
| 1635 | ISLAGSYKDW | L52 | IYKTPPIKD | L9; L13 | LWAHGFEL | L61; L59; L60; |
| | | | | | | L56 |
| 1636 | ISMATNYDL | L53 | IYKTPPIKDF | L9; L8 | LYDANYFLC | L62; L1; L9; L12 |
| 1637 | ISPYNSQNAV | L57 | IYLYLTFYLT | L9; L8 | LYDKLQFTSL | L62; L9; L68; L8 |
| 1638 | ISRQRLTK | L13 | IYNDKVAG | L71; L72 | LYFIKGLNNL | L9; L8; L71; L72 |
| 1639 | ISSDVLVN | L73 | IYNDKVAGFAK | L9; L8 | LYIIKLIFLW | L9; L8; L52; L12 |
| | | | F | | | |
| 1640 | ISTCACEI | L55 | IYQAGSTP | L71; L72 | LYLDAYNMM | L71; L72; L8; L9 |
| 1641 | ISTEIYQAG | L43 | IYQAGSTPC | L71; L72 | LYLTFYLTN | L8; L9; L71; L72 |
| 1642 | ITDAQTGS | L1 | IYQTSNFR | L71; L72 | LYNKYKYF | L9; L62; L72; L71 |
| 1643 | ITDVFYKE | L1 | IYSLLLCRM | L62; L9 | LYNSASFSTF | L8; L9; L72; L71 |
| 1644 | ITEEVGHTDLM | L18 | IYSTAALGV | L72; L71 | LYYQNNVFM | L72; L71; L62; L9 |
| 1645 | ITFELDERIDK | L7 | IYSTAALGVL | L9; L8 | MADQAMTQMY | L1; L18; L31; L49 |
| 1646 | ITGLYPTL | L67 | IYTELEPPCRF | L9; L8 | MAFPSGKVE | L69; L70; L61; |
| | | | | | | L32 |
| 1647 | ITGNTLQCI | L53 | KACPLIAA | L50; L51 | MEKLKTLV | L45; L33; L23; |
| | | | | | | L48 |
| 1648 | ITLATCEL | L60 | KAFKQIVESC | L54; L52 | MEKLKTLVA | L42; L46; L38; |
| | | | | | | L37 |
| 1649 | ITLATCELYHY | L12 | KAIVSTIQRKY | L14; L52 | MELTPVVQTI | L45; L40; L41; |
| | | | | | | L36 |
| 1650 | ITNCVKML | L73 | KALRKVPTD | L54; L52 | MGYINVFAF | L27; L26; L47; |
| | | | | | | L59 |
| 1651 | ITNLCPFGEVF | L52 | KATEETFKLSY | L14; L18 | MIDVQQWGF | L63; L1; L68; L54 |
| 1652 | ITPCSFGGV | L57 | KCDRAMPNML | L63; L68 | MIERFVSL | L60; L63; L68; |
| | | | | | | L23 |
| 1653 | ITPGTNTSN | L57 | KCKSAFYIL | L39; L13 | MLDNRATL | L62; L63; L68; L5 |
| 1654 | ITREVGFVVP | L52 | KDAPYIVGD | L65; L33 | MPLGYVTHGL | L31; L32; L56; |
| | | | | | | L22 |
| 1655 | ITSGDGTTS | L53 | KDKSAQCF | L33; L55 | MRIFTIGTV | L35; L64; L30; |
| | | | | | | L28 |
| 1656 | ITSKETLYC | L18 | KDLSPRWY | L55; L14 | MRNAGIVGV | L30; L64; L35; |
| | | | | | | L25 |
| 1657 | ITSKETLYCI | L53 | KDMTYRRL | L33; L55 | MRNAGIVGVL | L65; L35; L66; |
| | | | | | | L30 |
| 1658 | ITTDNTFV | L73 | KDVVECLKL | L33; L37 | MSDVKCTSV | L63; L68; L18; L1 |
| 1659 | ITTYPGQGLNGY | L1 | KEDLKFPRG | L45; L36 | MVSLLSVL | L59; L67; L25; |
| | | | | | | L74 |
| 1660 | ITVATSRTLSY | L18 | KEELDKYFK | L36; L38 | MWLIINLV | L48; L47; L25; |
| | | | | | | L33 |
| 1661 | ITVEELKK | L7 | KEGFFTYI | L45; L33 | MWLSYFIASF | L9; L8; L12; L10 |
| 1662 | ITVEELKKLL | L53 | KEGQINDM | L33; L37 | MYMGTLSY | L71; L72; L66; |
| | | | | | | L12 |
| 1663 | IVAAIVFIT | L20 | KEGQINDMI | L45; L36 | NANQVIVNN | L49; L17; L31; |
| | | | | | | L19 |
| 1664 | IVAGGIVAIVV | L20 | KEGSSVELKHF | L41; L40 | NAVASKIL | L70; L60; L61; |
| | | | | | | L69 |
| 1665 | IVAIVVTCLAYY | L18 | KEGVEFLRD | L45; L65 | NDPVGFTL | L33; L57; L66; |
| | | | | | | L71 |
| 1666 | IVEEAKKV | L63 | KENDSKEGFFTY | L40; L14 | NEFACVVAD | L29; L46; L37; |
| | | | | | | L42 |
| 1667 | IVGDVVQEGV | L20 | KESVQTFF | L33; L29 | NEYRLYLDA | L42; L46; L38; |
| | | | | | | L25 |
| 1668 | IVGGQIVTC | L26 | KETLYCIDGA | L38; L42 | NFCALILAY | L12; L72; L71; |
| | | | | | | L31 |
| 1669 | IVGVALLAVF | L52 | KFKEGVEFLR | L15; L21 | NFKNLREF | L71; L72; L62; |
| | | | | | | L23 |
| 1670 | IVKFISTCA | L51 | KFLKTNCCRF | L8; L9 | NFVFPLNSI | L8; L71; L9; L72 |
| 1671 | IVNNWLKQLIK | L6 | KFLPFQQFGR | L15; L21 | NFVRIIMRL | L20; L8; L29; L9 |
| 1672 | IVNSVLLFLAF | L12 | KFNGLTVLP | L8; L62 | NGVEGENCY | L29; L49; L27; |
| | | | | | | L10 |
| 1673 | IVNVSLVKPSF | L52 | KFNPPALQDAY | L14; L12 | NIKPVPEV | L3; L48; L23; L58 |
| 1674 | IVQLSEISM | L28 | KFTDGVCLFW | L8; L9 | NKWGKARLY | L27; L64; L14; |
| | | | | | | L66 |
| 1675 | IVQMLSDTL | L67 | KFVRIQPGQTF | L8; L9 | NLAKHCLHV | L3; L24; L2; L4 |
| 1676 | IVRFPNITNL | L13 | KFYDAQPC | L72; L71 | NLDSKVGGNY | L1; L18; L14; L12 |
| 1677 | IVSTIQRKYK | L13 | KGRLIIREN | L55; L13 | NLEEAARY | L1; L18; L12; L14 |
| 1678 | IVVTCLAY | L59 | KGSLPINVI | L16; L44 | NLIDSYFVVK | L6; L19; L7; L13 |
| 1679 | IVWHVNNATNK | L6 | KGVEAVMY | L54; L27 | NLKTLLSL | L23; L3; L25; L57 |
| 1680 | IVYTACSHAA | L51 | KGYHLMSF | L54; L59 | NLKTLLSLR | L15; L17; L21; |
| | | | | | | L19 |
| 1681 | IWLGFIAGLI | L8 | KHFSMMIL | L28; L35 | NLLEILQKE | L4; L24; L2; L5 |
| 1682 | IWNLDYIINL | L62 | KHITSKETLY | L14; L18 | NLPGCDGGSLY | L14; L12; L11; |
| | | | | | | L18 |
| 1683 | IWNVKDFM | L62 | KHLIPLMY | L27; L18 | NLQSNHDLY | L1; L18; L12; L14 |
| 1684 | IYSKHTPIN | L9 | KHSSGVTR | L21; L15 | NPETNILL | L39; L56; L23; |
| | | | | | | L32 |
| 1685 | IYTELEPPC | L9 | KHSSGVTREL | L34; L28 | NPFMIDVQQ | L49; L32; L31; |
| | | | | | | L50 |
| 1686 | KAHFPREGV | L13 | KHTPINLVR | L35; L21 | NPKAIKCV | L39; L23; L51; |
| | | | | | | L47 |
| 1687 | KAHKDKSAQ | L43 | KHVYQLRAR | L15; L21 | NPNYEDLLI | L49; L32; L34; |
| | | | | | | L47 |
| 1688 | KAHKDKSAQCF | L54 | KIADKYVR | L21; L15 | NQFNSAIGK | L7; L19; L13; L6 |
| 1689 | KAIDGGVT | L55 | KIADKYVRN | L43; L16 | NQPYPNASF | L27; L26; L57; L9 |
| 1690 | KAIDGGVTRD | L54 | KIITLKKR | L21; L15 | NQVIVNNL | L48; L25; L35; |
| | | | | | | L28 |
| 1691 | KALRKVPTDNY | L14 | KILGLPTQT | L6; L16 | NRGMVLGSL | L66; L25; L35; |
| | | | | | | L30 |
| 1692 | KARLYYDSM | L43 | KIQDSLSST | L14; L16 | NRNRFLYII | L35; L34; L64; |
| | | | | | | L30 |
| 1693 | KASCTLSEQL | L54 | KITEHSWNADL | L14; L18 | NRPQIGVVR | L66; L19; L30; |
| | | | Y | | | L35 |
| 1694 | KASMPTTIAK | L6 | KIVNNWLKQL | L16; L60 | NSTLEQYVF | L53; L54; L55; |
| | | | | | | L52 |
| 1695 | KAYKDYLAS | L43 | KIYSKHTPINL | L16; L6 | NTCDGTTF | L53; L54; L70; |
| | | | | | | L58 |
| 1696 | KAYNVTQA | L69 | KKFDTFNGE | L27; L44 | NTKHSSGVTR | L17; L19; L15; |
| | | | | | | L21 |
| 1697 | KAYNVTQAFGR | L15 | KKIKACVEE | L44; L27 | NTSNQVAVLY | L12; L18; L1; L14 |
| 1698 | KCAYWVPR | L21 | KKKADETQAL | L44; L27 | NTWCIRCLW | L52; L16; L54; |
| | | | | | | L53 |
| 1699 | KCDHCGETSW | L54 | KKLLEQWNL | L44; L27 | NTYLEGSVR | L17; L19; L21; |
| | | | | | | L15 |
| 1700 | KCDRAMPNM | L63 | KKPNELSRV | L5; L24 | NVAFELWAK | L7; L19; L6; L17 |
| 1701 | KCEESSAKSA | L38 | KKPNELSRVL | L44; L66 | NVLTLVYKV | L4; L20; L24; L5 |
| 1702 | KCKSAFYI | L55 | KKQQTVTLL | L44; L27 | NVLTLVYKVY | L10; L14; L12; |
| | | | | | | L11 |
| 1703 | KCRSKNPLL | L13 | KKVDGVVQ | L27; L44 | NYITTYPGQGL | L8; L72; L71; L9 |
| 1704 | KCSRIIPAR | L21 | KKVDGVVQQ | L27; L44 | NYLGKPREQ | L71; L72; L8; L17 |
| 1705 | KDASGKPVP | L46 | KKVDGVVQQL | L44; L27 | NYLKRRVV | L71; L25; L23; |
| | | | | | | L72 |
| 1706 | KDASGKPVPY | L43 | KKVKPTVVV | L44; L27 | NYLKSPNF | L9; L8; L71; L72 |
| 1707 | KDATPSDF | L33 | KKWKYPQVN | L44; L27 | NYNYLYRL | L66; L62; L8; L71 |
| 1708 | KDATPSDFVR | L21 | KLALGGSVAI | L16; L2 | NYQKVGMQK | L17; L13; L71; L8 |
| 1709 | KDCPAVAK | L33 | KLCEEMLDNR | L21; L15 | NYQVNGYPNMF | L9; L8; L72; L71 |
| 1710 | KDCPAVAKH | L33 | KLDGFMGR | L21; L15 | NYSGVVTTVM | L66; L71; L72; L8 |
| 1711 | KDEDDNLIDSY | L18 | KLDGFMGRIR | L21; L15 | NYTVSCLPF | L72; L9; L71; L8 |
| 1712 | KDFMSLSE | L33 | KLDGVVCTE | L5; L2 | NYYKKDNSYF | L8; L71; L9; L72 |
| 1713 | KDFYDFAV | L33 | KLDNDALNN | L18; L5 | PAFDKSAF | L43; L59; L58; |
| | | | | | | L70 |
| 1714 | KDFYDFAVSK | L13 | KLDNYYKKDNS | L18; L1 | PAFDKSAFV | L20; L58; L69; |
| | | | Y | | | L70 |
| 1715 | KDGHVETFY | L14 | KLFAAETLKA | L3; L2 | PEEHFIETI | L45; L37; L36; |
| | | | | | | L42 |
| 1716 | KDGIIWVA | L65 | KLGSLVVR | L21; L15 | PFFSNVTWF | L9; L72; L8; L71 |
| 1717 | KDGTCGLV | L33 | KLHDELTGHML | L2; L16 | PINPTDQSSY | L43; L14; L26; |
| | | | | | | L18 |
| 1718 | KDHIGTRN | L33 | KLINIIIWELL | L4; L16 | PLIAAVITR | L21; L19; L17; |
| | | | | | | L15 |
| 1719 | KDKKKKAD | L23 | KLKKSLNVA | L3; L51 | PLIQPIGAL | L4; L10; L3; L11 |
| 1720 | KDKSAQCFK | L13 | KLKKSLNVAK | L13; L6 | PLNSIIKTI | L24; L3; L4; L16 |
| 1721 | KDLLARAG | L33 | KLKPVLDW | L16; L54 | PLVDLPIGI | L3; L2; L4; L5 |
| 1722 | KDLPKEIT | L33 | KLKVDTANPK | L13; L6 | PTLNISDEF | L53; L52; L9; L12 |
| 1723 | KDMTYRRLI | L55 | KLLHKPIVWHV | L4; L2 | PYCYDTNVL | L9; L8; L72; L71 |
| 1724 | KDMTYRRLISM | L33 | KLLKSIAATR | L21; L15 | PYNSVTSSI | L9; L8; L72; L71 |
| 1725 | KDPNFKDQV | L33 | KLMGHFAW | L16; L54 | QAGNVQLRV | L73; L69; L24; |
| | | | | | | L70 |
| 1726 | KDQVILLN | L33 | KLQFTSLEI | L24; L16 | QEAYEQAVA | L42; L38; L46; |
| | | | | | | L40 |
| 1727 | KDTEKYCAL | L33 | KLQNNELSPV | L2; L3 | QEGVLTAV | L45; L33; L48; |
| | | | | | | L37 |
| 1728 | KDVLECNV | L33 | KLQSSQAW | L54; L16 | QEGVLTAVVI | L40; L41; L36; |
| | | | | | | L45 |
| 1729 | KDYLASGG | L33 | KLRSDVLLPL | L6; L3 | QEHYVRITG | L42; L41; L38; |
| | | | | | | L29 |
| 1730 | KEELDKYF | L33 | KLSYGIATVR | L21; L15 | QEHYVRITGL | L37; L41; L36; |
| | | | | | | L40 |
| 1731 | KEELDKYFKN | L38 | KLWAQCVQLH | L6; L16 | QELGKYEQYI | L40; L45; L41; |
| | | | | | | L36 |
| 1732 | KEEVKPFI | L45 | KMFDAYVNT | L13; L16 | QELYSPIFLI | L41; L40; L36; |
| | | | | | | L45 |
| 1733 | KEEVKPFIT | L38 | KMFYKGVITH | L16; L6 | QGDDYVYL | L63; L62; L68; |
| | | | | | | L67 |
| 1734 | KEGATTCGY | L14 | KMKDLSPR | L15; L21 | QIDRLITGR | L21; L17; L19; |
| | | | | | | L15 |
| 1735 | KEGATTCGYL | L36 | KMQRMLLEK | L13; L6 | QLCQYLNTL | L4; L3; L5; L57 |
| 1736 | KEGQINDMIL | L36 | KMSDVKCTSV | L3; L2 | QLIKVTLV | L48; L3; L24; L23 |
| 1737 | KEGQINDMILSL | L36 | KMVSLLSV | L48; L73 | QLLFVVEV | L4; L2; L23; L48 |
| 1738 | KEGSSVELK | L36 | KNADIVEEA | L3; L46 | QPILLLDQAL | L22; L32; L31; |
| | | | | | | L56 |
| 1739 | KEIKESVQTFF | L36 | KNIDGYFKIY | L14; L27 | QPRVEKKKL | L39; L22; L23; |
| | | | | | | L56 |
| 1740 | KEILVTYNCC | L38 | KNKCVNFNF | L53; L52 | QPVSELLTP | L32; L51; L50; |
| | | | | | | L31 |
| 1741 | KEITVATS | L37 | KNLREFVF | L54; L53 | QRNAPRITF | L30; L65; L34; |
| | | | | | | L66 |
| 1742 | KELLVYAADP | L38 | KPFLNKVVS | L56; L50 | QSTQWSLFF | L73; L53; L55; |
| | | | | | | L54 |
| 1743 | KENDSKEGFF | L40 | KPFLNKVVST | L56; L50 | QTIEVNSFSGY | L11; L14; L1; L10 |
| 1744 | KENSYTTTIKP | L38 | KPGNFNKDF | L39; L56 | QTYVTQQL | L55; L70; L48; |
| | | | | | | L25 |
| 1745 | KEPCSSGTY | L14 | KPHNSHEGKTF | L39; L56 | QTYVTQQLI | L55; L48; L24; |
| | | | | | | L53 |
| 1746 | KESPFELE | L33 | KPIVWHVNNA | L51; L50 | QVRDVLVRGF | L10; L11; L52; |
| | | | | | | L26 |
| 1747 | KESPFELEDFI | L36 | KPLEFGATSA | L51; L50 | QWNLVIGFLF | L9; L40; L41; L8 |
| 1748 | KESVQTFFKLV | L45 | KPPISFPL | L22; L56 | QYELKHGTF | L9; L62; L8; L72 |
| 1749 | KFADDLNQLTGY | L14 | KPSFYVYSR | L21; L15 | QYGSFCTQL | L9; L66; L72; L8 |
| 1750 | KFCLEASFNY | L12 | KPSKRSFIED | L56; L39 | QYIDIGNY | L72; L71; L14; |
| | | | | | | L66 |
| 1751 | KFDEDDSE | L62 | KPTETICA | L51; L50 | QYIKWPWYI | L9; L8; L64; L55 |
| 1752 | KFDEDDSEP | L62 | KPTETICAPL | L56; L22 | QYNRYLALY | L14; L12; L72; L9 |
| 1753 | KFDTFNGE | L62 | KPTVVVNA | L51; L50 | RAFDIYNDK | L13; L7; L6; L19 |
| 1754 | KFDTFNGEC | L62 | KQDDKKIKA | L24; L63 | RAMPNMLRIM | L69; L70; L52; |
| | | | | | | L55 |
| 1755 | KFISTCAC | L71 | KQEILGTV | L48; L24 | RDLSLQFKR | L15; L21; L30; L7 |
| 1756 | KFISTCACEI | L8 | KQFDTYNLWNT | L16; L27 | RDVLVRGF | L33; L65; L37; |
| | | | F | | | L29 |
| 1757 | KFKTEGLCV | L13 | KQGNFKNLR | L21; L15 | REEAIRHVRA | L38; L42; L46; |
| | | | | | | L37 |
| 1758 | KFLKTNCCR | L15 | KQIYKTPPI | L48; L24 | REFLTRNPA | L46; L38; L42; |
| | | | | | | L37 |
| 1759 | KFLPFQQFG | L8 | KQYGDCLGD | L27; L24 | REGVFVSN | L33; L45; L37; |
| | | | | | | L29 |
| 1760 | KFLTENLLLYI | L8 | KRAKVTSAM | L66; L30 | RELKVTFFP | L38; L42; L46; |
| | | | | | | L33 |
| 1761 | KFLVFLGI | L8 | KRHTFSNY | L66; L65 | RELNGGAYTR | L21; L36; L15; |
| | | | | | | L38 |
| 1762 | KFLVFLGII | L8 | KRISNCVADY | L30; L14 | REPMLQSA | L33; L37; L38; |
| | | | | | | L46 |
| 1763 | KFNPPALQ | L62 | KRNVIPTI | L48; L66 | RFPNITNLC | L8; L9; L72; L71 |
| 1764 | KFNPPALQDAYY | L14 | KRRVVFNGV | L13; L30 | RGDKSVYY | L18; L1; L54; L27 |
| 1765 | KFPRGQGV | L71 | KRSFIEDL | L65; L66 | RHINAQVAK | L7; L28; L13; L6 |
| 1766 | KFYDAQPCSDK | L6 | KRSFIEDLLF | L30; L34 | RHSLSHFVNL | L34; L56; L28; |
| | | | | | | L35 |
| 1767 | KFYGGWHNMLK | L6 | KSAQCFKM | L54; L55 | RIFTIGTV | L48; L24; L3; L33 |
| 1768 | KGAKLLHKP | L46 | KSAQCFKMFY | L18; L14 | RIFTIGTVTLK | L6; L7; L13; L21 |
| 1769 | KGDYGDAVV | L63 | KSATCITR | L21; L15 | RIMASLVL | L73; L61; L60; |
| | | | | | | L56 |
| 1770 | KGFCDLKGK | L13 | KSAYENFNQ | L54; L53 | RIMASLVLAR | L21; L6; L7; L15 |
| 1771 | KGFCDLKGKY | L14 | KSDGTGTIYT | L18; L1 | RKYKGIKI | L24; L27; L48; |
| | | | | | | L44 |
| 1772 | KGGAPTKVTF | L65 | KSDGTGTIYTEL | L18; L68 | RLANECAQV | L3; L24; L2; L4 |
| 1773 | KGGRTIAF | L54 | KSIAATRGA | L46; L55 | RLFRKSNLK | L6; L21; L13; L7 |
| 1774 | KGIKIQEGV | L24 | KSLNVAKSE | L55; L53 | RLQSLENV | L4; L24; L2; L3 |
| 1775 | KGIMMNVAKY | L14 | KSNHNFLV | L73; L18 | RNFYEPQII | L24; L55; L65; |
| | | | | | | L73 |
| 1776 | KGLDYKAF | L27 | KSNHNFLVQ | L73; L18 | RNRDVDTDF | L27; L26; L65; |
| | | | | | | L53 |
| 1777 | KGLDYKAFK | L13 | KSNIIRGWI | L55; L73 | RQALLKTVQF | L27; L44; L26; |
| | | | | | | L16 |
| 1778 | KGLNNLNR | L15 | KSTNLVKNK | L13; L7 | RQCSGVTF | L27; L26; L44; |
| | | | | | | L54 |
| 1779 | KGLPWNVV | L48 | KSVLYYQNNVF | L54; L52 | RQFHQKLL | L48; L44; L24; |
| | | | | | | L27 |
| 1780 | KGLYRKCVKSR | L15 | KSWMESEFR | L21; L15 | RQGTDYKHW | L54; L16; L52; |
| | | | | | | L14 |
| 1781 | KGPITDVFY | L14 | KSYVHVVDG | L73; L53 | RQIAPGQTG | L24; L26; L27; |
| | | | | | | L44 |
| 1782 | KGPKVKYLYF | L8 | KTHVQLSLP | L18; L53 | RQKKQQTVTL | L44; L26; L27; |
| | | | | | | L56 |
| 1783 | KGVAPGTAV | L51 | KTILRKGGR | L21; L15 | RQVVNVVTT | L44; L26; L27; |
| | | | | | | L24 |
| 1784 | KGVAPGTAVL | L44 | KTPEEHFI | L55; L53 | RQWLPTGTLL | L44; L27; L30; |
| | | | | | | L24 |
| 1785 | KGVEAVMYM | L69 | KTPKYKFVR | L21; L15 | RRVVFNGVSF | L30; L65; L54; |
| | | | | | | L53 |
| 1786 | KGVHFVCNL | L69 | KTQFNYYKK | L13; L7 | RSEDKRAKV | L63; L73; L18; |
| | | | | | | L13 |
| 1787 | KHADFDTW | L54 | KTTEVVGD | L54; L55 | RSQMEIDFL | L55; L73; L53; |
| | | | | | | L65 |
| 1788 | KHAFLCLFLL | L34 | KTTEVVGDIILK | L6; L7 | RSVASQSI | L55; L54; L53; |
| | | | | | | L48 |
| 1789 | KHDFFKFRI | L34 | KTTVASLI | L55; L54 | RTIAFGGCVFSY | L18; L14; L16; |
| | | | | | | L52 |
| 1790 | KHFYWFFSN | L16 | KTVQFCDAM | L54; L53 | RTLLTKGTL | L55; L53; L16; |
| | | | | | | L44 |
| 1791 | KHKHAFLCL | L13 | KTYERHSLS | L18; L73 | RTTNGDFLH | L54; L53; L52; L7 |
| 1792 | KIADYNYK | L6 | KVATVQSK | L13; L6 | RVDFCGKGY | L18; L14; L1; L26 |
| 1793 | KIALKGGKI | L24 | KVGGNYNYLYR | L21; L15 | RVESSSKLW | L54; L18; L52; |
| | | | | | | L16 |
| 1794 | KIFVDGVPFVV | L16 | KVPATVSV | L5; L57 | RVQPTESIVR | L21; L15; L7; L13 |
| 1795 | KILNNLGV | L73 | KVTIDYTEISF | L16; L52 | RVVTTFDSEY | L14; L18; L26; |
| | | | | | | L54 |
| 1796 | KINAACRKV | L73 | KVTKGKAKK | L13; L6 | RYFRLTLGV | L24; L13; L8; L71 |
| 1797 | KIVDEPEEHV | L2 | KVYYGNALD | L14; L21 | RYMNSQGL | L71; L72; L8; L9 |
| 1798 | KKAGGTTE | L27 | KWADNNCY | L54; L18 | RYRIGNYKL | L8; L9; L13; L66 |
| 1799 | KKAGGTTEML | L44 | KWDLIISD | L62; L68 | RYWEPEFYEA | L8; L13; L9; L65 |
| 1800 | KKCKSAFY | L27 | KWDLTAFGLV | L62; L18 | SAAKKNNL | L74; L23; L56; |
| | | | | | | L58 |
| 1801 | KKCKSAFYIL | L44 | KWPWYIWLGF | L8; L9 | SAFAMMFVK | L7; L6; L19; L69 |
| 1802 | KKGAKLLHK | L6 | KYFSGAMDTTS | L14; L71 | SAMQTMLF | L58; L59; L74; |
| | | | Y | | | L70 |
| 1803 | KKKLDGFMGR | L15 | KYFVKIGPER | L15; L21 | SAMVRMYIF | L59; L58; L69; |
| | | | | | | L70 |
| 1804 | KKLKKSLNV | L73 | KYLPIDKCSRI | L8; L9 | SCVLSGHNL | L60; L36; L56; |
| | | | | | | L34 |
| 1805 | KKRWQLAL | L27 | KYLVQQESP | L71; L8 | SDIDITFLK | L7; L19; L6; L13 |
| 1806 | KKSTNLVKN | L44 | KYNENGTITD | L8; L9 | SDIDITFLKK | L7; L13; L19; L6 |
| 1807 | KLALGGSVAIK | L6 | KYPQVNGLTS | L8; L9 | SDIDYVPLK | L7; L13; L6; L19 |
| 1808 | KLASHMYCSFY | L18 | KYPQVNGLTSI | L9; L8 | SDNGPQNQR | L15; L21; L17; |
| | | | | | | L19 |
| 1809 | KLDDKDPNFK | L6 | KYVRNLQHRL | L8; L9 | SDYDYYRY | L29; L65; L43; |
| | | | | | | L14 |
| 1810 | KLDGVVCT | L63 | LAAECTIFK | L7; L19 | SEAFLIGC | L33; L45; L37; |
| | | | | | | L29 |
| 1811 | KLDNDALN | L18 | LAAIMQLFFSY | L31; L12 | SEDNQTTTIQTI | L40; L45; L36; |
| | | | | | | L41 |
| 1812 | KLDNYYKK | L18 | LAATKMSEC | L70; L58 | SEPVLKGV | L33; L48; L45; |
| | | | | | | L37 |
| 1813 | KLDNYYKKD | L5 | LAATVRLQA | L50; L51 | SEPVLKGVKL | L36; L41; L37; |
| | | | | | | L40 |
| 1814 | KLEGYAFEH | L18 | LACEDLKPV | L47; L70 | SETKCTLKSF | L40; L41; L29; |
| | | | | | | L37 |
| 1815 | KLEGYAFEHI | L18 | LACFVLAAVY | L31; L12 | SEVGPEHSLA | L38; L42; L46; |
| | | | | | | L41 |
| 1816 | KLEGYAFEHIVY | L18 | LADNKFALT | L63; L1 | SFLAHIQWM | L71; L72; L12; L8 |
| 1817 | KLGASQRV | L24 | LAHIQWMVMF | L31; L49 | SFNPETNI | L62; L48; L71; |
| | | | | | | L72 |
| 1818 | KLGSLVVRCSF | L16 | LAIDAYPLT | L61; L31 | SFPLCANGQVF | L72; L71; L9; L8 |
| 1819 | KLHDELTGHM | L14 | LAIDAYPLTK | L7; L61 | SFRLFARTR | L17; L15; L21; |
| | | | | | | L48 |
| 1820 | KLHNWNCVN | L16 | LAKALNDF | L58; L59 | SFSGYLKL | L62; L8; L72; L71 |
| 1821 | KLIANQFNSA | L46 | LAKDTTEA | L43; L50 | SFYVYSRV | L48; L62; L71; |
| | | | | | | L72 |
| 1822 | KLINIIIWFLLL | L6 | LAKHCLHV | L70; L47 | SFYVYSRVK | L13; L71; L72; L7 |
| 1823 | KLKALNLGETF | L16 | LALITLAT | L59; L47 | SGAMDTTSY | L43; L26; L27; |
| | | | | | | L14 |
| 1824 | KLKKSLNV | L13 | LALLSDLQDL | L31; L60 | SIIIGGAKLK | L7; L19; L13; L6 |
| 1825 | KLLEQWNLVIGF | L16 | LANECAQV | L47; L70 | SIVCRFDTR | L19; L17; L21; |
| | | | | | | L15 |
| 1826 | KLLGVGGK | L6 | LAPLLSAG | L57; L59 | SLDTYPSLE | L1; L62; L63; L18 |
| 1827 | KLLGVGGKP | L14 | LAPLLSAGI | L47; L57 | SLENVAFNV | L4; L5; L2; L24 |
| 1828 | KLLHKPIVWH | L6 | LATHGLAA | L50; L61 | SLETIQITI | L24; L5; L2; L68 |
| 1829 | KLMGHFAWWTA | L16 | LCDRRATCF | L63; L62 | SLIDLQELGK | L6; L7; L13; L19 |
| 1830 | KLMGHFAWWTA | L16 | LCEKALKY | L1; L18 | SLIDLQELGKY | L14; L11; L12; |
| | F | | | | | L10 |
| 1831 | KLMVVIPDYNTY | L18 | LCPFGEVF | L72; L71 | SLKVPATVSV | L43; L3; L2; L13 |
| 1832 | KLNEEIAIILA | L2 | LDEWSMATY | L18; L1 | SLNGVTLI | L24; L3; L74; L48 |
| 1833 | KLNTDHSSS | L16 | LDEWSMATYY | L1; L18 | SLRPDTRY | L26; L43; L27; |
| | | | | | | L14 |
| 1834 | KLNVGDYF | L62 | LDISASIVA | L46; L38 | SLVKPSFYV | L5; L2; L4; L20 |
| 1835 | KLNVGDYFVL | L16 | LDSCKRVL | L65; L33 | SMDNSPNL | L62; L68; L63; |
| | | | | | | L28 |
| 1836 | KLQDVVNQNA | L2 | LDSFKEEL | L33; L65 | SMMGFKMNY | L26; L14; L12; |
| | | | | | | L16 |
| 1837 | KLQNNELSP | L16 | LDWLEEKF | L33; L29 | SMQNCVLKL | L4; L3; L73; L8 |
| 1838 | KLRGTAVMSLK | L6 | LEASFNYL | L45; L29 | SNSGSDVLY | L14; L12; L27; |
| | | | | | | L18 |
| 1839 | KLRSDVLL | L74 | LEDEFTPF | L29; L62 | SPDAVTAY | L31; L49; L1; L22 |
| 1840 | KLSHQSDIEV | L2 | LEGETLPTEV | L45; L38 | SPFELEDF | L31; L49; L56; |
| | | | | | | L39 |
| 1841 | KLTCATTR | L21 | LEGYAFEHI | L45; L41 | SPFHPLAD | L50; L51; L65; |
| | | | | | | L49 |
| 1842 | KLTDNVYI | L2 | LEILDITP | L38; L46 | SPFHPLADN | L51; L49; L56; |
| | | | | | | L22 |
| 1843 | KLVLSVNPYV | L2 | LEILDITPC | L38; L29 | SPIQYIDIGNY | L31; L11; L14; |
| | | | | | | L49 |
| 1844 | KLVSSFLEMK | L6 | LEILDITPCSF | L40; L41 | SPNECNQMC | L49; L32; L56; |
| | | | | | | L39 |
| 1845 | KMADQAMTQ | L16 | LEIPRRNVAT | L38; L46 | SPNECNQMCL | L56; L39; L32; |
| | | | | | | L22 |
| 1846 | KMKDLSPRWYF | L16 | LEKCDLQNY | L29; L26 | SPNLAWPLI | L47; L22; L50; |
| | | | | | | L39 |
| 1847 | KMSECVLGQ | L16 | LELAMDEF | L29; L33 | SPTKLNDL | L56; L39; L22; |
| | | | | | | L23 |
| 1848 | KMVSLLSVLL | L44 | LEPPCRFV | L45; L64 | SRELKVTF | L65; L35; L66; |
| | | | | | | L34 |
| 1849 | KNLSDRVV | L73 | LEQWNLVIGF | L40; L41 | SRLDKVEA | L65; L25; L35; |
| | | | | | | L66 |
| 1850 | KNSKVQIGEY | L14 | LEQYVFCTVNA | L38; L42 | SSAKSASVYY | L18; L1; L58; L14 |
| 1851 | KNTVKSVGK | L13 | LETAQNSVRV | L45; L38 | SSQGSEYDY | L14; L1; L18; L26 |
| 1852 | KPASRELKVTF | L39 | LETIQITISSF | L40; L41 | SSVLNDILSR | L19; L7; L15; L17 |
| 1853 | KPGGTSSGD | L65 | LFARTRSM | L72; L71 | STECSNLLL | L1; L18; L68; L63 |
| 1854 | KPIVWHVNN | L56 | LFDESGEFK | L62; L1 | STFEEAALCTF | L16; L52; L10; |
| | | | | | | L53 |
| 1855 | KPLEFGATSAA | L51 | LFDMSKFPL | L62; L68 | STFISAAR | L21; L15; L19; |
| | | | | | | L17 |
| 1856 | KPNELSRVLGL | L56 | LFFSYFAV | L71; L72 | STLEQYVF | L53; L54; L52; |
| | | | | | | L55 |
| 1857 | KPNTWCIRC | L56 | LFIRQEEV | L48; L71 | STVLSFCAF | L10; L52; L60; |
| | | | | | | L59 |
| 1858 | KPPPGDQFKHL | L39 | LFLAFVVFLL | L8; L9 | SVAYESLR | L19; L21; L17; |
| | | | | | | L15 |
| 1859 | KPREQIDGYVM | L39 | LFLALITLA | L50; L12 | SVIDLLLDD | L11; L7; L12; L49 |
| 1860 | KPRSQMEI | L39 | LFSTVFPPTSF | L9; L8 | SVPWDTIANY | L11; L10; L12; |
| | | | | | | L14 |
| 1861 | KPRSQMEIDF | L39 | LFTRFFYVL | L8; L9 | SVQTFFKLV | L13; L20; L73; |
| | | | | | | L64 |
| 1862 | KPSKRSFIEDL | L56 | LFVTVYSH | L71; L72 | SVSSPDAVTAY | L14; L6; L11; L12 |
| 1863 | KPTETICAP | L51 | LFVTVYSHLL | L8; L9 | SVTSNYSGV | L11; L3; L10; L20 |
| 1864 | KPVPEVKI | L47 | LFYSYATH | L71; L72 | SVVNARLR | L19; L15; L17; |
| | | | | | | L21 |
| 1865 | KPVSEEVV | L51 | LFYSYATHS | L72; L71 | SYEDQDAL | L72; L71; L62; |
| | | | | | | L66 |
| 1866 | KPYIKWDLLK | L6 | LGDELGTDPY | L1; L18 | SYFIASFR | L17; L71; L15; |
| | | | | | | L72 |
| 1867 | KQARSEDKR | L21 | LGDVRETMSY | L18; L1 | SYLTPGDSS | L71; L72; L9; L8 |
| 1868 | KQASLNGV | L24 | LGFIAGLI | L47; L48 | SYYKLGASQR | L17; L15; L19; |
| | | | | | | L21 |
| 1869 | KQASLNGVT | L44 | LGLAAIMQL | L4; L61 | SYYSLLMPI | L71; L8; L72; L9 |
| 1870 | KQATKYLV | L24 | LGSLAATV | L47; L48 | TACTDDNAL | L32; L67; L57; |
| | | | | | | L68 |
| 1871 | KQATKYLVQ | L44 | LGVLVPHV | L47; L48 | TAFGLVAEWF | L31; L49; L52; |
| | | | | | | L58 |
| 1872 | KQIRSAAKK | L24 | LGYVTHGL | L25; L47 | TAHSCNVNR | L17; L19; L15; |
| | | | | | | L21 |
| 1873 | KQIVESCGNF | L26 | LHAPATVC | L28; L35 | TANKWDLII | L32; L47; L73; |
| | | | | | | L49 |
| 1874 | KQIYKTPPIK | L13 | LHNDILLAK | L6; L7 | TASDTYACW | L49; L54; L52; |
| | | | | | | L10 |
| 1875 | KQLPFFYYS | L15 | LIAIVMVTI | L47; L24 | TCGQQQTTL | L34; L35; L28; |
| | | | | | | L65 |
| 1876 | KQQTVTLLP | L24 | LIANQFNSA | L46; L50 | TCLAYYFMR | L17; L15; L21; L7 |
| 1877 | KQRRPQGL | L44 | LIIMRTFKV | L4; L5 | TDPSFLGRY | L14; L11; L12; |
| | | | | | | L10 |
| 1878 | KQVEQKIAE | L44 | LIKVTLVFLF | L52; L12 | TEDDYQGKP | L38; L42; L46; |
| | | | | | | L45 |
| 1879 | KQYGDCLGDI | L24 | LIPLMYKGL | L57; L5 | TEEVGHTDL | L36; L25; L37; |
| | | | | | | L45 |
| 1880 | KRDAPAHI | L63 | LIVAAIVF | L59; L60 | TEHSWNADLY | L41; L29; L14; |
| | | | | | | L40 |
| 1881 | KRFKESPF | L66 | LKAPKEIIFL | L44; L64 | TEISFMLW | L41; L40; L29; |
| | | | | | | L45 |
| 1882 | KRHTFSNYQH | L30 | LKFNPPAL | L66; L59 | TENKYSQL | L33; L37; L29; |
| | | | | | | L45 |
| 1883 | KRISNCVAD | L30 | LKFPRGQGV | L25; L64 | TENLLLYI | L45; L48; L33; |
| | | | | | | L41 |
| 1884 | KRNIKPVP | L65 | LKPANNSL | L57; L66 | TENLLLYID | L42; L29; L40; |
| | | | | | | L41 |
| 1885 | KRNIKPVPE | L30 | LKQLIKVTL | L44; L56 | TENLTKEGA | L42; L38; L46; |
| | | | | | | L37 |
| 1886 | KRNIKPVPEV | L30 | LKQLPFFYY | L27; L12 | TERLKLFAA | L42; L46; L38; |
| | | | | | | L37 |
| 1887 | KRNIKPVPEVK | L30 | LLADKFPVLH | L6; L3 | TESNKKFL | L33; L37; L45; |
| | | | | | | L36 |
| 1888 | KRNRATRVE | L65 | LLAKDTTEA | L3; L2 | TEVNEFACVV | L38; L37; L45; |
| | | | | | | L36 |
| 1889 | KRNVIPTIT | L30 | LLAKDTTEAF | L26; L43 | TFCAGSTF | L72; L71; L62; L9 |
| 1890 | KRNVIPTITQ | L30 | LLAPLLSAG | L3; L4 | TFISAARQGF | L8; L9; L12; L72 |
| 1891 | KRNVIPTITQM | L30 | LLEKCDLQNY | L1; L18 | TFLKKDAPY | L12; L71; L72; |
| | | | | | | L14 |
| 1892 | KRPINPTD | L66 | LLFLAFVV | L47; L23 | TFYLTNDV | L48; L71; L62; |
| | | | | | | L72 |
| 1893 | KRRVVFNGVSF | L30 | LLKDCPAVA | L3; L51 | TFYPKLQSS | L8; L43; L72; L71 |
| 1894 | KRTATKAY | L66 | LLKSAYENF | L43; L26 | THDVSSAI | L28; L35; L68; |
| | | | | | | L34 |
| 1895 | KRTIKGTHH | L30 | LLLCRMNSR | L21; L15 | TIAEILLIIMR | L19; L21; L17; |
| | | | | | | L15 |
| 1896 | KRVDWTIEYP | L65 | LLLDDFVEII | L4; L2 | TIKKPNEL | L23; L39; L58; |
| | | | | | | L74 |
| 1897 | KRVLNVVCK | L30 | LLLDQALVSD | L4; L2 | TKHSSGVTR | L17; L19; L21; |
| | | | | | | L15 |
| 1898 | KRWQLALSKG | L30 | LLMPILTLTR | L21; L6 | TLACFVLAA | L3; L46; L2; L4 |
| 1899 | KRWQLALSKGV | L30 | LLPLTQYNRY | L12; L1 | TLACFVLAAV | L3; L2; L4; L20 |
| 1900 | KSAFVNLK | L55 | LLPLVSSQC | L57; L5 | TLATCELYHY | L12; L10; L18; |
| | | | | | | L14 |
| 1901 | KSAFYILPSI | L52 | LLPSLATVA | L5; L57 | TLATHGLAA | L3; L51; L46; L2 |
| 1902 | KSAPLIELCV | L73 | LLQFAYANR | L21; L15 | TLATHGLAAV | L3; L4; L2; L20 |
| 1903 | KSAQCFKMFYK | L7 | LLQLCTFTR | L21; L15 | TLAVPYNMR | L21; L19; L17; |
| | | | | | | L15 |
| 1904 | KSDGTGTIYTE | L18 | LLQNGMNGR | L21; L15 | TLEETKFL | L62; L63; L23; |
| | | | | | | L68 |
| 1905 | KSEDAQGMD | L18 | LLSKGRLII | L24; L16 | TLGVLVPHV | L4; L2; L5; L3 |
| 1906 | KSEDAQGMDNL | L18 | LLSVCLGSLIY | L18; L12 | TLKGVEAVMY | L26; L12; L14; |
| | A | | | | | L43 |
| 1907 | KSEFDRDAA | L18 | LLSVLQQLR | L21; L15 | TLNDFNLVA | L2; L3; L46; L4 |
| 1908 | KSEFDRDAAM | L18 | LLTNMFTPL | L5; L74 | TLNDFNLVAM | L4; L2; L3; L43 |
| 1909 | KSEKQVEQK | L18 | LLWPVTLA | L2; L3 | TLPKGIMMNV | L5; L57; L2; L3 |
| 1910 | KSEKQVEQKI | L53 | LLWPVTLACF | L5; L12 | TLSEQLDFI | L3; L4; L2; L5 |
| 1911 | KSFDLGDELG | L52 | LMCQPILL | L74; L62 | TMADLVYALR | L19; L21; L15; |
| | | | | | | L17 |
| 1912 | KSFTVEKG | L54 | LMRELNGGAY | L14; L43 | TNPIQLSSY | L26; L72; L14; |
| | | | | | | L71 |
| 1913 | KSHFAIGLALYY | L18 | LNDLCFTNVY | L1; L18 | TPCGTGTST | L51; L39; L22; |
| | | | | | | L32 |
| 1914 | KSIAATRG | L54 | LNDNLLEI | L68; L62 | TPCNGVEGF | L49; L31; L34; |
| | | | | | | L39 |
| 1915 | KSILSPLYA | L73 | LNHTKKWKY | L12; L14 | TPEEHFIET | L31; L32; L39; |
| | | | | | | L49 |
| 1916 | KSKCEESSAK | L13 | LNIIPLTTA | L50; L46 | TPEEHFIETI | L49; L34; L39; |
| | | | | | | L32 |
| 1917 | KSLTENKYS | L52 | LNRVCGVSA | L51; L50 | TPFDVVRQ | L49; L47; L50; |
| | | | | | | L56 |
| 1918 | KSPIQYIDI | L53 | LNSIIKTI | L48; L47 | TPFEIKLA | L50; L51; L47; |
| | | | | | | L23 |
| 1919 | KSPIQYIDIGNY | L18 | LPAPRTLLTK | L22; L6 | TPGSGVPV | L51; L50; L39; |
| | | | | | | L22 |
| 1920 | KSQDLSVVS | L53 | LPDDFTGCVI | L49; L32 | TPKGPKVKYL | L39; L56; L22; |
| | | | | | | L32 |
| 1921 | KSTNLVKN | L54 | LPETTADIV | L32; L47 | TPLIQPIGAL | L22; L56; L31; |
| | | | | | | L32 |
| 1922 | KSYELQTPFEI | L55 | LPFFSNVT | L47; L50 | TPNNTDFSR | L32; L19; L17; |
| | | | | | | L15 |
| 1923 | KTCGQQQTTLK | L6 | LPFGWLIVGVA | L50; L51 | TPRDLGACI | L39; L51; L49; |
| | | | | | | L22 |
| 1924 | KTDGTLMIE | L18 | LPFKLTCA | L50; L51 | TPSFKKGAKL | L56; L39; L32; |
| | | | | | | L22 |
| 1925 | KTGDLQPLEQ | L73 | LPFNDGVYFA | L50; L51 | TPSKLIEY | L31; L49; L56; |
| | | | | | | L39 |
| 1926 | KTILRKGGRTI | L55 | LPFQQFGRD | L49; L50 | TQHQPYVV | L48; L35; L24; |
| | | | | | | L28 |
| 1927 | KTIQPRVE | L55 | LPFQQFGRDI | L39; L47 | TQLYLGGMSY | L27; L14; L26; |
| | | | | | | L12 |
| 1928 | KTKNVTKEN | L13 | LPGCDGGSLY | L31; L18 | TQYNRYLALY | L14; L27; L26; |
| | | | | | | L12 |
| 1929 | KTLLSLREVRTI | L55 | LPGVFCGVDA | L50; L51 | TRAKVGIL | L35; L66; L65; |
| | | | | | | L64 |
| 1930 | KTNCCRFQ | L55 | LPKGIMMNVA | L51; L50 | TRELMREL | L25; L35; L64; |
| | | | | | | L66 |
| 1931 | KTNCCRFQE | L16 | LPLQLGFST | L50; L31 | TRNPAWRKA | L64; L30; L51; |
| | | | | | | L65 |
| 1932 | KTNCCRFQEK | L13 | LPNDDTLRVEA | L50; L51 | TSAVLQSGF | L53; L52; L54; |
| | | | | | | L26 |
| 1933 | KTPEEHFIETI | L16 | LPPKNSIDA | L51; L50 | TSDYYQLY | L1; L18; L54; L55 |
| 1934 | KTPKYKFVRI | L53 | LPPKNSIDAF | L39; L56 | TSFGPLVR | L19; L17; L7; L15 |
| 1935 | KTSVDCTM | L54 | LPRVFSAVG | L51; L22 | TSRTLSYY | L1; L18; L14; L58 |
| 1936 | KTSVDCTMYI | L53 | LPSLATVA | L50; L51 | TSTDVVYRAF | L10; L52; L55; |
| | | | | | | L43 |
| 1937 | KTTLPVNV | L73 | LPSYAAFA | L50; L51 | TTEILPVSM | L63; L18; L1; L62 |
| 1938 | KTTLPVNVA | L51 | LPSYAAFATA | L50; L51 | TTITVNVLAW | L10; L52; L54; |
| | | | | | | L19 |
| 1939 | KTTVASLINTL | L16 | LPTEVLTE | L47; L50 | TTRQVVNVV | L20; L13; L70; |
| | | | | | | L73 |
| 1940 | KTVGELGD | L65 | LPTEVLTEEV | L50; L51 | TTSPISEHDY | L14; L18; L12; L1 |
| 1941 | KTVGELGDVR | L21 | LPTGVHAGTDL | L56; L22 | TTTNIVTR | L19; L17; L21; |
| | | | | | | L15 |
| 1942 | KTVQFCDAMR | L21 | LPTMCDIRQL | L39; L32 | TVATSRTLSYY | L11; L14; L10; |
| | | | | | | L18 |
| 1943 | KVDGVVQQ | L63 | LPVNVAFELW | L49; L31 | TVCGMWKGY | L26; L14; L10; |
| | | | | | | L11 |
| 1944 | KVDTANPKT | L63 | LPYPDPSRILGA | L50; L51 | TVDSSQGSEY | L1; L18; L14; L10 |
| 1945 | KVDTANPKTPK | L6 | LQDLKWARF | L63; L62 | TVEEAKTV | L23; L39; L48; |
| | | | | | | L63 |
| 1946 | KVDTANPKTPKY | IL18 | LQELGKYEQY | L18; L1 | TVEELKKL | L39; L63; L23; |
| | | | | | | L68 |
| 1947 | KVEAEVQI | L63 | LQIPFAMQMAY | L27; L26 | TVGELGDVR | L19; L17; L21; |
| | | | | | | L15 |
| 1948 | KVFTTVDNINL | L16 | LQTYVTQQL | L44; L24 | TVYDDGARRV | L20; L11; L14; |
| | | | | | | L24 |
| 1949 | KVGGSCVL | L65 | LRAKHYVY | L66; L65 | TYACWHHSI | L8; L9; L71; L72 |
| 1950 | KVKPTVVVNAA | L51 | LRAKHYVYI | L64; L34 | TYHPNCVNCL | L72; L8; L71; L9 |
| 1951 | KVKYLYFI | L55 | LRDGWEIVK | L30; L35 | TYLDGADVT | L8; L72; L71; L9 |
| 1952 | KVKYLYFIKG | L13 | LREVRTIKV | L25; L64 | TYLEGSVR | L17; L15; L71; |
| | | | | | | L72 |
| 1953 | KVLLRKNGNK | L6 | LRKGGRTI | L55; L65 | TYPGQGLNG | L8; L72; L9; L71 |
| 1954 | KVNINIVGDF | L26 | LRPDTRYV | L64; L66 | VAEWFLAY | L59; L18; L1; L31 |
| 1955 | KVQHMVVK | L13 | LRSDVLLP | L65; L64 | VAEWFLAYI | L47; L68; L63; |
| | | | | | | L69 |
| 1956 | KVQIGEYTFEK | L7 | LRVEAFEY | L66; L65 | VAFNVVNK | L7; L13; L19; L70 |
| 1957 | KVTSAMQTMLF | L16 | LRVIGHSM | L25; L66 | VAGFAKFL | L70; L74; L60; |
| | | | | | | L61 |
| 1958 | KVVSTTTN | L54 | LSAPTLVPQEHY | L1; L52 | VAGGIVAIVV | L69; L70; L58; |
| | | | | | | L67 |
| 1959 | KVVSTTTNIVTR | L19 | LSAQTGIAV | L61; L50 | VAIKITEH | L43; L59; L74; |
| | | | | | | L58 |
| 1960 | KWDLLKYD | L62 | LSDTLKNLS | L1; L18 | VAIKITEHSW | L52; L54; L49; |
| | | | | | | L53 |
| 1961 | KWDLTAFG | L62 | LSDTLKNLSD | L1; L18 | VAIVVTCLAY | L43; L12; L59; |
| | | | | | | L31 |
| 1962 | KWKYPQVNG | L13 | LSEARQHL | L63; L1 | VATSRTLSYY | L58; L14; L69; |
| | | | | | | L12 |
| 1963 | KWYIRVGARK | L6 | LSEARQHLK | L1; L18 | VAVPTGYV | L70; L58; L47; |
| | | | | | | L69 |
| 1964 | KYDFTEER | L62 | LSEMVMCGGSL | L1; L18 | VCLSGLDSL | L67; L4; L8; L44 |
| | | | Y | | | |
| 1965 | KYFDCYDGGCI | L8 | LSETKCTL | L63; L68 | VCRHHANEY | L43; L59; L26; |
| | | | | | | L58 |
| 1966 | KYFSGAMDT | L71 | LSFELLHAP | L46; L50 | VDADSKIVQL | L56; L37; L4; L3 |
| 1967 | KYKFVRIQP | L13 | LSKGRLII | L55; L70 | VDGVDVELF | L9; L33; L52; L34 |
| 1968 | KYLPIDKC | L8 | LSKGVHFV | L70; L58 | VDSSQGSEY | L14; L26; L43; |
| | | | | | | L18 |
| 1969 | KYLPIDKCS | L8 | LSKGVHFVC | L70; L52 | VEAPLVGTP | L46; L38; L42; |
| | | | | | | L29 |
| 1970 | KYLPIDKCSR | L15 | LSKSLTENKY | L14; L1 | VEFLRDGWEI | L45; L36; L40; |
| | | | | | | L41 |
| 1971 | KYLYFIKGLNNL | L8 | LSLLSKGRLI | L55; L52 | VELFENKTT | L38; L45; L41; |
| | | | | | | L40 |
| 1972 | KYPQVNGLT | L9 | LSMQGAVDI | L55; L53 | VENMTPRDL | L37; L36; L40; |
| | | | | | | L45 |
| 1973 | KYTQLCQY | L14 | LSPVALRQM | L57; L59 | VEQKIAEI | L45; L48; L33; |
| | | | | | | L37 |
| 1974 | KYVRNLQHRLY | L14 | LSRLDKVEA | L50; L43 | VETFYPKL | L37; L45; L29; |
| | | | | | | L36 |
| 1975 | KYWDQTYH | L71 | LSTDGNKI | L55; L48 | VETKAIVST | L37; L45; L38; |
| | | | | | | L29 |
| 1976 | KYWDQTYHPNC | L8 | LSVCLGSL | L59; L60 | VETVKGLDY | L29; L41; L40; |
| | | | | | | L12 |
| 1977 | LAAVYRINWI | L47 | LSVCLGSLIY | L18; L1 | VFCGVDAVNL | L62; L8; L72; L71 |
| 1978 | LACFVLAA | L50 | LSVLQQLRV | L73; L48 | VFFDGRVD | L72; L71; L65; |
| | | | | | | L55 |
| 1979 | LADKFPVLHDI | L68 | LSVVSKVV | L48; L70 | VFISPYNSQ | L8; L72; L71; L9 |
| 1980 | LAFLLFLVLIM | L31 | LSWEVGKPR | L15; L19 | VFLFVAAIFY | L12; L72; L71; |
| | | | | | | L14 |
| 1981 | LAHAEETRKL | L32 | LTAVVIPTKK | L6; L7 | VFLHVTYV | L72; L71; L62; |
| | | | | | | L48 |
| 1982 | LAHIQWMV | L47 | LTDEMIAQ | L1; L68 | VFLVLLPL | L71; L72; L59; |
| | | | | | | L62 |
| 1983 | LAKNVSLDN | L43 | LTDNVYIKN | L1; L18 | VFMSEAKCW | L71; L72; L9; L8 |
| 1984 | LALCADSI | L47 | LTEEVVLKT | L1; L18 | VFNICQAV | L71; L48; L72; |
| | | | | | | L62 |
| 1985 | LALCADSII | L47 | LTKGTLEPEY | L52; L14 | VFQSASKI | L48; L71; L72; |
| | | | | | | L62 |
| 1986 | LALCADSIII | L47 | LTLVYKVY | L52; L55 | VGPEHSLAEY | L43; L57; L14; |
| | | | | | | L12 |
| 1987 | LALGGSVA | L50 | LTNNVAFQTV | L73; L20 | VGVALLAVF | L9; L31; L52; L27 |
| 1988 | LALLSDLQDLKW | L52 | LTPLGIDL | L57; L62 | VHFISNSWL | L28; L35; L34; |
| | | | | | | L25 |
| 1989 | LALSKGVHFV | L47 | LTPTWRVY | L55; L1 | VIDLLLDDF | L62; L1; L68; L63 |
| 1990 | LALYNKYKYF | L52 | LTPVYSFL | L57; L62 | VIPDYNTYK | L6; L7; L57; L5 |
| 1991 | LALYYPSA | L50 | LTQYNRYL | L55; L60 | VKCTSVVLL | L44; L66; L62; |
| | | | | | | L64 |
| 1992 | LALYYPSAR | L17 | LTQYNRYLALY | L1; L18 | VLAWLYAAV | L3; L2; L4; L5 |
| 1993 | LARAGKASC | L43 | LTSHTVMPL | L61; L60 | VLDMCASL | L68; L62; L63; L5 |
| 1994 | LATNNLVVMA | L50 | LTSLLVLV | L73; L1 | VLHDIGNPK | L6; L13; L43; L7 |
| 1995 | LATNNLVVMAY | L31 | LTVLPPLL | L60; L55 | VLIMLIIF | L74; L26; L23; |
| | | | | | | L59 |
| 1996 | LAVHECFV | L47 | LTWICLLQFAY | L1; L12 | VLLPLTQYN | L4; L12; L2; L5 |
| 1997 | LAVHECFVKR | L19 | LTYNKVENM | L70; L58 | VLLPLVSSQ | L4; L16; L3; L2 |
| 1998 | LAVPYNMRVIHF | L31 | LVAAGLEA | L51; L50 | VLPFNDGVYF | L9; L12; L57; L5 |
| 1999 | LAYILFTRFFY | L12 | LVDFQVTIA | L68; L63 | VLTESNKKF | L9; L26; L16; L12 |
| 2000 | LCADSIII | L47 | LVDFQVTIAEI | L5; L68 | VPATVSVSS | L51; L56; L39; |
| | | | | | | L22 |
| 2001 | LCANGQVF | L59 | LVDLPIGIN | L68; L1 | VPFWITIAYI | L20; L47; L49; |
| | | | | | | L50 |
| 2002 | LCFLAFLL | L47 | LVDLPIGINI | L68; L63 | VPHVGEIPV | L51; L50; L47; |
| | | | | | | L22 |
| 2003 | LCNSQTSL | L57 | LVDSDLNDFV | L5; L1 | VPLNIIPLT | L47; L50; L31; |
| | | | | | | L51 |
| 2004 | LCNSQTSLR | L21 | LVFLFVAA | L50; L51 | VPTDNYITT | L49; L56; L50; |
| | | | | | | L51 |
| 2005 | LCVDIPGI | L47 | LVFLFVAAIFY | L12; L31 | VPTDNYITTY | L49; L31; L43; |
| | | | | | | L12 |
| 2006 | LDDFVEII | L68 | LVIGFLFLT | L12; L20 | VPVAIHADQL | L31; L32; L56; |
| | | | | | | L39 |
| 2007 | LDERIDKVL | L32 | LVKPSFYVYSR | L15; L21 | VPYNMRVI | L47; L48; L39; |
| | | | | | | L25 |
| 2008 | LDGEVITFD | L65 | LVLIMLIIFWF | L52; L12 | VQEGVLTAV | L24; L48; L68; |
| | | | | | | L63 |
| 2009 | LDGISQYSL | L65 | LVLVQSTQW | L52; L54 | VQLSLPVL | L48; L25; L44; |
| | | | | | | L59 |
| 2010 | LDMYSVML | L33 | LVQAGNVQLR | L21; L19 | VQPQLEMEL | L57; L44; L64; L5 |
| 2011 | LDSKTQSL | L33 | LVQMAPISA | L50; L51 | VQPTESIVRF | L26; L9; L8; L27 |
| 2012 | LDSKVGGNY | L14 | LVRGFGDSV | L51; L58 | VRDPQTLEIL | L35; L34; L66; |
| | | | | | | L68 |
| 2013 | LDYIINLII | L47 | LVSDIDITFLK | L6; L7 | VRFPNITN | L25; L66; L65; |
| | | | | | | L35 |
| 2014 | LDYKAFKQI | L47 | LVSDVGDSA | L50; L51 | VRGTTVLL | L66; L65; L64; |
| | | | | | | L35 |
| 2015 | LEASFNYLK | L38 | LVSSFLEM | L59; L67 | VRGTTVLLK | L30; L65; L7; L6 |
| 2016 | LEASFNYLKSP | L46 | LVSSFLEMK | L6; L7 | VRIIMRLWL | L25; L64; L66; |
| | | | | | | L30 |
| 2017 | LEDEFTPFDV | L38 | LVSSQCVNL | L74; L56 | VSDVGDSAEV | L63; L68; L1; L18 |
| 2018 | LEETKFLTE | L46 | LVTLAILTA | L50; L51 | VSELLTPL | L68; L59; L63; L1 |
| 2019 | LEGETLPTEVL | L36 | LWAKRNIKP | L38; L46 | VSFCYMHHM | L70; L73; L58; |
| | | | | | | L69 |
| 2020 | LEGSVAYESL | L36 | LWLDDVVY | L71; L31 | VSIWNLDY | L1; L43; L18; L54 |
| 2021 | LEGSVRVVT | L38 | LWPVTLAC | L71; L57 | VSLDNVLST | L43; L55; L52; |
| | | | | | | L73 |
| 2022 | LEGYAFEHIVY | L41 | LYAFASEA | L72; L71 | VSTTTNIVTR | L19; L15; L7; L21 |
| 2023 | LEIPRRNVATL | L36 | LYCIDGAL | L72; L71 | VTGDSCNNY | L18; L1; L14; L58 |
| 2024 | LELAMDEFI | L36 | LYDKLVSSFL | L62; L63 | VTTTLEETKF | L52; L53; L54; |
| | | | | | | L12 |
| 2025 | LELQDHNETC | L36 | LYECLYRNR | L17; L15 | VTTVMFLAR | L7; L15; L17; L19 |
| 2026 | LEMELTPV | L45 | LYFDKAGQK | L13; L72 | VVADAVIKT | L20; L19; L7; L6 |
| 2027 | LEQPTSEA | L38 | LYFIKGLNN | L71; L72 | VVAFNTLLFL | L74; L20; L6; L60 |
| 2028 | LEQPTSEAVEA | L38 | LYIDINGNLH | L8; L9 | VVDKYFDCY | L18; L1; L12; L14 |
| 2029 | LEQPYVFI | L45 | LYKMQRMLL | L9; L71 | VVDMSMTY | L18; L1; L68; L63 |
| 2030 | LEQPYVFIK | L38 | LYLDAYNMMI | L8; L9 | VVENPTIQK | L7; L6; L13; L19 |
| 2031 | LEQWNLVI | L45 | LYLYALVYFL | L9; L8 | VVEVVDKYF | L63; L52; L62; |
| | | | | | | L12 |
| 2032 | LEQWNLVIGFLF | L40 | LYQDVNCT | L72; L71 | VVGDIILK | L7; L6; L13; L19 |
| 2033 | LESELVIGAVI | L41 | LYQDVNCTEV | L72; L71 | VVHNQDVNL | L74; L56; L67; |
| | | | | | | L63 |
| 2034 | LETAQNSVRVL | L41 | LYQPPQTS | L71; L72 | VVIGIVNNTVY | L14; L11; L12; |
| | | | | | | L26 |
| 2035 | LETIQITIS | L46 | LYRKCVKSR | L17; L15 | VVISSDVL | L67; L74; L60; |
| | | | | | | L61 |
| 2036 | LFDMSKFP | L62 | LYSPIFLIVA | L50; L51 | VVLSFELL | L74; L67; L60; |
| | | | | | | L23 |
| 2037 | LFDMSKFPLKL | L62 | LYVNKHAFH | L71; L72 | VVPGLPGTIL | L57; L67; L56; L5 |
| 2038 | LFDRYFKYW | L62 | LYYDSMSYE | L72; L71 | VVPGLPGTILR | L19; L21; L7; L15 |
| 2039 | LFFFLYENA | L50 | MAPISAMV | L47; L57 | VVTTKIAL | L23; L74; L56; |
| | | | | | | L60 |
| 2040 | LFKDCSKVI | L8 | MATNYDLSVV | L69; L70 | VVTTKIALK | L7; L6; L13; L19 |
| 2041 | LFLLPSLAT | L71 | MAYITGGV | L47; L48 | VVTTVMFLAR | L7; L19; L6; L15 |
| 2042 | LFLMSFTV | L71 | MAYRENGI | L47; L48 | VYCFLGYF | L9; L62; L72; L71 |
| 2043 | LFLPFFSNVTWF | L8 | MAYRFNGIGV | L50; L47 | VYCPRHVI | L9; L71; L8; L72 |
| 2044 | LFMRIFTI | L47 | MCASLKEL | L25; L59 | VYKQFDTY | L66; L14; L71; |
| | | | | | | L72 |
| 2045 | LFTMLRKL | L62 | MCASLKELL | L34; L20 | VYKVYYGNAL | L71; L72; L9; L66 |
| 2046 | LFVAAIFYLI | L8 | MDNSPNLAW | L49; L16 | VYQCAMRPNF | L9; L8; L71; L72 |
| 2047 | LFVVEVVDKY | L12 | MDSTVKNY | L29; L33 | VYSDVENPHL | L9; L8; L66; L72 |
| 2048 | LFWNCNVDR | L17 | MELPTGVH | L29; L33 | VYSHLLLVA | L9; L8; L50; L51 |
| 2049 | LFWNCNVDRY | L12 | MESEFRVYS | L42; L46 | VYYTSNPTT | L9; L71; L72; L8 |
| 2050 | LGERVRQAL | L32 | MESLVPGF | L33; L29 | WEIQQVVDA | L46; L38; L42; |
| | | | | | | L37 |
| 2051 | LGFIAGLIA | L50 | MEVTPSGTWLT | L41; L40 | WFSQRGGSY | L14; L71; L72; |
| | | | Y | | | L12 |
| 2052 | LGFSTGVNLV | L47 | MFITREEA | L71; L72 | WFVTQRNFY | L71; L12; L72; |
| | | | | | | L14 |
| 2053 | LGGLHLLI | L47 | MFITREEAI | L71; L72 | WLLWPVTL | L23; L74; L4; L2 |
| 2054 | LGGSVAIKI | L47 | MGHFAWWTA | L50; L51 | WLMWLIINLV | L3; L2; L4; L5 |
| 2055 | LGIITTVAA | L50 | MHHMELPTG | L28; L34 | WLTNIFGTVY | L12; L14; L26; |
| | | | | | | L43 |
| 2056 | LGLPTQTVD | L65 | MIELSLIDFY | L1; L18 | WSMATYYLF | L54; L53; L55; |
| | | | | | | L52 |
| 2057 | LGQSKRVD | L65 | MILSLLSK | L6; L7 | WVMRIMTWL | L20; L74; L60; |
| | | | | | | L10 |
| 2058 | LGRYMSAL | L59 | MILSLLSKGR | L17; L21 | YADVFHLYLQY | L1; L18; L49; L12 |
| 2059 | LGTDPYEDF | L49 | MISAGFSLW | L16; L10 | YAWNRKRI | L47; L70; L69; |
| | | | | | | L48 |
| 2060 | LGTEVNEF | L59 | MKDLSPRWY | L27; L1 | YEDQDALFA | L38; L46; L42; |
| | | | | | | L45 |
| 2061 | LHAPATVCG | L28 | MKFLVFLGI | L47; L27 | YEDQDALFAY | L1; L40; L41; L29 |
| 2062 | LHCANFNVLF | L34 | MLTNDNTSR | L21; L17 | YENAFLPFA | L46; L38; L42; |
| | | | | | | L50 |
| 2063 | LHDIGNPKA | L28 | MPASWVMRIM | L32; L22 | YENFNQHEVL | L37; L36; L41; |
| | | | | | | L29 |
| 2064 | LHDIGNPKAI | L28 | MPILTLTR | L50; L47 | YEPQIITTD | L45; L37; L36; |
| | | | | | | L29 |
| 2065 | LHFLPRVESA | L50 | MPILTLTRA | L50; L51 | YEQFKKGV | L45; L33; L38; |
| | | | | | | L37 |
| 2066 | LHLLIGLAK | L6 | MPLGYVTH | L50; L47 | YEQFKKGVQI | L45; L37; L38; |
| | | | | | | L36 |
| 2067 | LHNDILLA | L50 | MPLGYVTHG | L50; L31 | YEQYIKWPW | L40; L41; L38; |
| | | | | | | L42 |
| 2068 | LHPTQAPTHL | L57 | MPLSAPTLVP | L50; L51 | YFCTCYFGL | L72; L62; L8; L71 |
| 2069 | LHSSRLSF | L28 | MPNLYKMQRM | L32; L31 | YFIASFRL | L71; L72; L62; L8 |
| 2070 | LHTQVVDM | L28 | MPNMLRIM | L47; L32 | YFVLTSHTV | L71; L48; L72; |
| | | | | | | L62 |
| 2071 | LHVTYVPAQ | L28 | MPNMLRIMA | L50; L51 | YGVSPTKL | L74; L67; L25; |
| | | | | | | L61 |
| 2072 | LHVVGPNV | L28 | MPTTIAKNTV | L50; L32 | YHDVRVVLD | L34; L62; L28; |
| | | | | | | L35 |
| 2073 | LHVVGPNVN | L28 | MPYFFTLLLQ | L50; L47 | YICGFIQQK | L7; L13; L6; L19 |
| 2074 | LIAAVITREV | L3 | MQNCVLKL | L48; L73 | YIDINGNLH | L1; L68; L18; L63 |
| 2075 | LIDAMMFTSDL | L68 | MRIFTIGTVTL | L34; L35 | YIVDSVTVK | L19; L7; L6; L60 |
| 2076 | LIDFYLCFLAF | L68 | MRPNFTIKG | L64; L66 | YLALYNKY | L1; L26; L18; L27 |
| 2077 | LIDLQELG | L1 | MSALNHTKK | L7; L19 | YLAVFDKNLY | L1; L12; L18; L26 |
| 2078 | LIDLQELGK | L1 | MSDVKCTSVV | L63; L68 | YLDAYNMM | L62; L1; L63; L68 |
| 2079 | LIGCNYLGK | L6 | MSLSEQLRK | L7; L6 | YLEGSVRV | L2; L63; L1; L62 |
| 2080 | LIIKNLSKSL | L60 | MTNRQFHQK | L7; L13 | YLKLTDNVY | L26; L27; L1; L14 |
| 2081 | LIINLVQM | L59 | MTPRDLGAC | L10; L11 | YLQPRTFLLKY | L12; L1; L14; L18 |
| 2082 | LIINLVQMA | L3 | MTYGQQFGPTY | L14; L11 | YLQYIRKL | L64; L23; L4; L74 |
| 2083 | LIISVTSN | L43 | MVLGSLAA | L50; L51 | YNYLYRLF | L33; L27; L59; |
| | | | | | | L55 |
| 2084 | LINTLNDL | L59 | MVPHISRQRL | L20; L5 | YPANSIVCR | L19; L17; L49; |
| | | | | | | L31 |
| 2085 | LIRQGTDY | L26 | MVYMPASWV | L20; L47 | YPDPSRILGA | L50; L51; L49; |
| | | | | | | L32 |
| 2086 | LITPVHVMSK | L6 | MYASAVVLLI | L9; L8 | YPIIGDELKI | L47; L49; L32; |
| | | | | | | L34 |
| 2087 | LIVGVALLAV | L5 | MYDPKTKNV | L62; L5 | YPSARIVYT | L49; L32; L31; |
| | | | | | | L22 |
| 2088 | LIVNSVLLFL | L60 | MYIFFASFYY | L12; L14 | YQHEETIY | L26; L27; L43; |
| | | | | | | L58 |
| 2089 | LKALNLGETF | L27 | MYKGLPWNVV | L15; L17 | YRLFRKSNL | L25; L23; L30; |
| | | | R | | | L66 |
| 2090 | LKATEETF | L27 | MYMGTLSYEQF | L8; L9 | YRVTKNSKV | L64; L30; L25; |
| | | | | | | L35 |
| 2091 | LKATEETFKL | L44 | NAAISDYDY | L49; L31 | YRYNLPTMC | L64; L66; L25; |
| | | | | | | L30 |
| 2092 | LKEPCSSGTY | L27 | NAANVYLKH | L69; L19 | YSDSPCESH | L68; L43; L1; L63 |
| 2093 | LKGVEAVMY | L27 | NADIVEEAK | L67; L68 | YSDVENPHLM | L63; L1; L68; L62 |
| 2094 | LKKLKKSL | L23 | NALPETTAD | L31; L32 | YSGQSTQL | L74; L61; L60; |
| | | | | | | L65 |
| 2095 | LKLFAAETL | L44 | NARLRAKHY | L58; L70 | YSHLLLVAA | L50; L46; L67; |
| | | | | | | L51 |
| 2096 | LKLRSDVLL | L44 | NASSSEAF | L49; L54 | YSKWYIRV | L70; L43; L58; |
| | | | | | | L69 |
| 2097 | LKLTDNVY | L27 | NASVVNIQK | L19; L7 | YSNNSIAI | L48; L59; L43; |
| | | | | | | L61 |
| 2098 | LKNLSDRVVF | L27 | NCDVVIGI | L62; L68 | YTEISFMLW | L1; L52; L18; L49 |
| 2099 | LKRGDKSVYY | L27 | NCYLATALL | L28; L62 | YTMADLVY | L1; L18; L59; L70 |
| 2100 | LKYDFTEER | L27 | NDFNLVAMKY | L12; L29 | YTVEEAKTVL | L60; L53; L10; |
| | | | | | | L20 |
| 2101 | LKYDFTEERL | L44 | NDMILSLL | L33; L25 | YVDTPNNTD | L68; L63; L1; L32 |
| 2102 | LLADKFPV | L2 | NEEIAIILASF | L41; L40 | YVDTPNNTDF | L68; L63; L1; L60 |
| 2103 | LLAGTITSG | L3 | NEFYAYLRKH | L29; L41 | YVFTGYRVTK | L7; L13; L19; L6 |
| 2104 | LLAPLLSAGI | L3 | NEFYAYLRKHF | L41; L40 | YVLGLAAIM | L31; L12; L67; |
| | | | | | | L32 |
| 2105 | LLAVFQSASK | L6 | NEKQEILGTVSW | L40; L41 | YVLPNDDTLR | L17; L15; L21; |
| | | | | | | L19 |
| 2106 | LLDDFVEIIKS | L5 | NELSPVALRQ | L41; L40 | YVMHANYIFWR | L7; L21; L19; L15 |
| 2107 | LLDKRTTC | L63 | NELSPVALRQM | L40; L41 | YVTHGLNL | L60; L61; L74; |
| | | | | | | L59 |
| 2108 | LLDQALVS | L1 | NELSRVLGLKTL | L41; L40 | YYHKNNKSW | L71; L9; L72; L8 |
| 2109 | LLDQALVSDV | L5 | NESGLKTILR | L19; L41 | YYKLGASQR | L17; L15; L21; |
| | | | | | | L71 |
| 2110 | LLDRLNQLE | L1 | NETLVTMPLGY | L41; L40 | YYQNNVFM | L71; L72; L62; |
| | | | | | | L66 |
| 2111 | LLDRLNQLES | L1 | NEVAKNLNESL | L36; L41 | YYQNNVFMS | L72; L66; L71; L9 |
| 2112 | LLEQWNLV | L1 | NFGAISSV | L72; L71 | YYVGYLQPR | L17; L15; L71; |
| | | | | | | L72 |
| 2113 | LLEQWNLVI | L68 | NFKNLREFVF | L8; L9 | YYVGYLQPRTF | L9; L8; L72; L71 |
| 2114 | LLFLVLIM | L59 | NFLVQAGNV | L72; L71 | AAIVFITLC | L69; L74; L58; |
| | | | | | | L70; L7 |
| 2115 | LLFLVLIMLI | L3 | NFNFNGLTG | L72; L71 | AAIVFITLCF | L59; L52; L43; |
| | | | | | | L69; L12 |
| 2116 | LLFVTVYSH | L4 | NFTIKGSF | L72; L71 | AAKAYKDYL | L58; L67; L70; |
| | | | | | | L69; L74 |
| 2117 | LLFVVEVVDK | L6 | NFTTAPAI | L71; L72 | AAVITREV | L48; L70; L59; |
| | | | | | | L69; L64 |
| 2118 | LLFVVEVVDKY | L12 | NGDSEVVLK | L19; L7 | ACTDDNALAY | L12; L14; L40; |
| | | | | | | L18; L41 |
| 2119 | LLHAPATV | L3 | NGDVVAIDY | L1; L18 | ADAQSFLNR | L7; L15; L21; L19; |
| | | | | | | L17 |
| 2120 | LLHKPIVW | L23 | NGIGVTQNV | L24; L20 | AEAELAKN | L46; L45; L37; |
| | | | | | | L33; L42 |
| 2121 | LLHKPIVWH | L43 | NGLTGTGVL | L25; L44 | AEAELAKNVS | L46; L42; L40; |
| | | | | | | L36; L41 |
| 2122 | LLIGLAKR | L21 | NGMNGRTI | L55; L48 | AEHVNNSY | L29; L45; L33; |
| | | | | | | L26; L41 |
| 2123 | LLIIMRTFK | L6 | NGTITVEEL | L67; L25 | AEIVDTVSAL | L36; L40; L41; |
| | | | | | | L37; L45 |
| 2124 | LLKDCPAV | L3 | NGVSFSTF | L29; L27 | AENSVAYSN | L42; L38; L40; |
| | | | | | | L41; L37 |
| 2125 | LLKDCPAVAK | L6 | NGYPNMFI | L47; L25 | AENVTGLFK | L40; L7; L41; L6; |
| | | | | | | L42 |
| 2126 | LLKEPCSS | L43 | NHDLYCQV | L35; L28 | AEVAVKMF | L33; L29; L41; |
| | | | | | | L37; L40 |
| 2127 | LLKEPCSSG | L43 | NHNFLVQA | L35; L28 | AEWFLAYILF | L40; L41; L36; |
| | | | | | | L37; L42 |
| 2128 | LLKEPCSSGT | L43 | NIDGYFKI | L68; L63 | AFEKMVSLL | L62; L72; L63; |
| | | | | | | L71; L8 |
| 2129 | LLKEPCSSGTY | L14 | NIFGTVYEKL | L11; L10 | AFGLVAEWF | L72; L9; L62; L71; |
| | | | | | | L12 |
| 2130 | LLKSIAAT | L43 | NIIPLTTAAK | L19; L6 | AFLIGCNYL | L71; L72; L8; L9; |
| | | | | | | L62 |
| 2131 | LLLDKRTT | L23 | NILLNVPL | L23; L25 | AFYILPSII | L62; L72; L71; |
| | | | | | | L48; L69 |
| 2132 | LLLFVTVY | L26 | NIQKEIDRL | L4; L34 | AGFSLWVY | L27; L69; L14; |
| | | | | | | L26; L59 |
| 2133 | LLLLDRLNQL | L4 | NITFELDER | L19; L17 | AIDGGVTR | L21; L63; L68; |
| | | | | | | L15; L19 |
| 2134 | LLLQLCTF | L23 | NITRFQTL | L60; L23 | AISDYDYYR | L21; L15; L7; L19; |
| | | | | | | L17 |
| 2135 | LLLQLCTFTR | L21 | NIVTRCLNR | L19; L17 | AIVSTIQR | L21; L19; L7; L15; |
| | | | | | | L17 |
| 2136 | LLLTILTSLL | L4 | NKSWMESEF | L27; L66 | AIVSTIQRK | L7; L6; L19; L24; |
| | | | | | | L13 |
| 2137 | LLLVAAGL | L23 | NKWDLIISD | L44; L27 | ALDPLSET | L1; L68; L5; L62; |
| | | | | | | L63 |
| 2138 | LLMPLKAPK | L6 | NLAWPLIVTA | L3; L2 | ALIISVTSNY | L26; L14; L6; L18; |
| | | | | | | L12 |
| 2139 | LLNKEMYL | L74 | NLDKSAGF | L63; L62 | ALIWNVKDF | L26; L10; L27; |
| | | | | | | L16; L11 |
| 2140 | LLNKEMYLKL | L4 | NLDSKVGGNYN | L18; L1 | ALLEDEFTPF | L6; L12; L9; L2; |
| | | | Y | | | L16 |
| 2141 | LLNRYFRLTL | L4 | NLEEAARYM | L62; L63 | ALNNIINNA | L3; L4; L2; L5; L24 |
| 2142 | LLPLTQYNR | L21 | NLGERVRQA | L3; L2 | ALPETTADI | L57; L5; L3; L24; |
| | | | | | | L2 |
| 2143 | LLPLTQYNRYL | L57 | NLGETFVTH | L5; L2 | ALYNKYKY | L14; L12; L26; |
| | | | | | | L18; L16 |
| 2144 | LLPLVSSQ | L57 | NLKPFERDI | L64; L3 | ALYYPSARIVY | L27; L6; L16; L14; |
| | | | | | | L12 |
| 2145 | LLPLVSSQCV | L5 | NLKQLPFFY | L12; L14 | AMYTPHTV | L48; L3; L69; L70; |
| | | | | | | L47 |
| 2146 | LLPPKNSID | L5 | NLKQLPFFYY | L12; L14 | APHGHVMV | L51; L39; L56; |
| | | | | | | L22; L50 |
| 2147 | LLSDLQDLK | L6 | NLLEILQKEKV | L2; L4 | APHGVVFL | L56; L22; L39; |
| | | | | | | L65; L23 |
| 2148 | LLSDLQDLKW | L12 | NLLLLFVTVY | L12; L27 | APISAMVRM | L56; L22; L32; |
| | | | | | | L31; L39 |
| 2149 | LLSKGRLI | L55 | NLNRGMVL | L23; L57 | APKEIIFL | L56; L39; L23; |
| | | | | | | L22; L51 |
| 2150 | LLSKGRLIIR | L21 | NLPFKLTCA | L3; L5 | AQEKNFTTA | L42; L46; L27; |
| | | | | | | L24; L26 |
| 2151 | LLSLREVRTI | L3 | NLQHRLYEC | L23; L4 | AQFAPSASAF | L26; L27; L16; |
| | | | | | | L71; L28 |
| 2152 | LLSTDGNKI | L24 | NLREFVFKN | L4; L3 | AQVDVVNF | L26; L27; L58; |
| | | | | | | L33; L69 |
| 2153 | LLSVLLSM | L59 | NLREMLAHA | L3; L2 | AQYELKHGT | L24; L27; L14; |
| | | | | | | L44; L26 |
| 2154 | LLTILTSLL | L74 | NLSDRVVF | L23; L26 | AQYELKHGTF | L27; L26; L14; |
| | | | | | | L44; L10 |
| 2155 | LLVAAGLEA | L51 | NLTTRTQL | L23; L25 | ARAGEAANF | L30; L34; L66; |
| | | | | | | L65; L35 |
| 2156 | LLVDSDLNDF | L26 | NNLVVMAY | L71; L29 | ARFYFYTSK | L30; L7; L13; L66; |
| | | | | | | L6 |
| 2157 | LLVTLAILTA | L3 | NPETNILLN | L49; L32 | ARSVASQSI | L35; L34; L28; |
| | | | | | | L64; L30 |
| 2158 | LLYDANYFLCW | L6 | NPHLMGWDY | L31; L49 | ASAFFGMSR | L7; L19; L15; L21; |
| | | | | | | L17 |
| 2159 | LMIERFVSLA | L3 | NPKAIKCVP | L39; L51 | ASCDAIMTR | L7; L21; L19; L15; |
| | | | | | | L17 |
| 2160 | LMNVLTLVYK | L6 | NPKGFCDL | L39; L23 | ASVYYSQL | L74; L60; L61; |
| | | | | | | L70; L67 |
| 2161 | LMSFPQSA | L50 | NPPALQDAY | L31; L12 | ASWVMRIMTW | L52; L53; L54; |
| | | | | | | L16; L10 |
| 2162 | LMSNLGMPSY | L12 | NPTDQSSYI | L49; L47 | ATEETFKLSY | L1; L18; L12; L14; |
| | | | | | | L16 |
| 2163 | LMWLSYFI | L47 | NQEYADVFHLY | L14; L1 | ATVHTANKW | L54; L52; L53; |
| | | | | | | L10; L16 |
| 2164 | LMWLSYFIA | L50 | NQHEVLLA | L24; L25 | ATVVIGTSK | L7; L19; L6; L13; |
| | | | | | | L15 |
| 2165 | LMYKGLPWNV | L3 | NQHEVLLAP | L24; L25 | ATYKPNTW | L54; L52; L16; |
| | | | | | | L55; L53 |
| 2166 | LNDILSRLD | L65 | NQRNAPRITF | L27; L26 | AVANGDSEV | L3; L20; L2; L58 |
| | | | | | | L28 |
| 2167 | LNGDVVAID | L65 | NRARTVAGV | L35; L30 | AVDCALDPL | L68; L5; L67; L63; |
| | | | | | | L62 |
| 2168 | LNGDVVAIDY | L12 | NRDVDTDF | L62; L35 | AVFQSASKI | L24; L16; L10; |
| | | | | | | L45; L3 |
| 2169 | LNGGAYTRY | L12 | NRDVDTDFV | L34; L35 | AVHFISNSW | L16; L10; L54; |
| | | | | | | L52; L26 |
| 2170 | LNGYTVEEA | L65 | NRFLYIIKLI | L30; L64 | AVKLQNNEL | L26; L68; L39; |
| | | | | | | L56; L67 |
| 2171 | LNIKLLGV | L73 | NRFNVAIT | L25; L35 | AVNLLTNMF | L26; L16; L12; |
| | | | | | | L52; L58 |
| 2172 | LNKEMYLKL | L73 | NRFTTTLNDF | L30; L34 | AYWVPRASA | L72; L42; L71; |
| | | | | | | L46; L51 |
| 2173 | LNTLTLAV | L48 | NRVCGVSA | L25; L35 | AYYVGYLQP | L46; L72; L42; |
| | | | | | | L71; L38 |
| 2174 | LPDDFTGCVIA | L50 | NRVVISSDV | L25; L35 | CAKEIKESV | L43; L70; L58; |
| | | | | | | L23; L69 |
| 2175 | LPDPSKPSKRSF | L39 | NRYFRLTLG | L25; L30 | CASEYTGNY | L58; L49; L14; |
| | | | | | | L18; L70 |
| 2176 | LPETTADIVV | L32 | NRYFRLTLGV | L25; L30 | CIMSDRDLY | L14; L18; L12; L1; |
| | | | | | | L16 |
| 2177 | LPFAMGII | L47 | NRYLALYNK | L30; L25 | CRFDTRVL | L65; L25; L64; |
| | | | | | | L66; L35 |
| 2178 | LPFAMGIIAMSA | L50 | NSASFSTFK | L7; L19 | CSFYEDFLEY | L18; L12; L1; L65; |
| | | | | | | L59 |
| 2179 | LPFFSNVTWFHA | L50 | NSFDVLKSE | L69; L70 | CTLSEQLDF | L53; L12; L52; |
| | | | | | | L54; L16 |
| 2180 | LPFFYYSD | L50 | NSGSDVLY | L1; L18 | CYMHHMEL | L71; L72; L25; |
| | | | | | | L66; L62 |
| 2181 | LPFNDGVY | L31 | NSLKITEEV | L73; L48 | DAIMTRCL | L23; L61; L70; |
| | | | | | | L47; L60 |
| 2182 | LPFQQFGRDIA | L50 | NSPRRARSV | L57; L64 | DAVNLLTNM | L11; L31; L19; |
| | | | | | | L58; L17 |
| 2183 | LPFTINCQ | L47 | NSQNAVASK | L19; L7 | DAVTAYNGY | L10; L11; L49; |
| | | | | | | L31; L19 |
| 2184 | LPFTINCQEP | L32 | NSTPGSSR | L19; L17 | DCSKVITGL | L20; L11; L34; |
| | | | | | | L25; L35 |
| 2185 | LPGVFCGV | L47 | NSVPWDTI | L67; L55 | DESGEFKLA | L42; L38; L46; |
| | | | | | | L41; L29 |
| 2186 | LPGVYSVI | L47 | NSWLMWLI | L48; L55 | DFVKATCEF | L8; L9; L72; L10; |
| | | | | | | L71 |
| 2187 | LPGVYSVIYLY | L31 | NTDFSRVSA | L68; L1 | DGARRVWTL | L25; L23; L10; |
| | | | | | | L29; L47 |
| 2188 | LPIDKCSRIIPA | L50 | NTDHSSSSD | L1; L18 | DIDITFLKK | L1; L6; L7; L18; |
| | | | | | | L19 |
| 2189 | LPIGINIT | L47 | NTLQCIMLV | L73; L20 | DILSRLDKV | L23; L11; L4; L47; |
| | | | | | | L25 |
| 2190 | LPKGFYAEG | L51 | NTLTLAVPY | L12; L14 | DKAYKIEEL | L44; L25; L23; |
| | | | | | | L20; L10 |
| 2191 | LPLQLGFSTG | L50 | NTPKDHIGTR | L11; L19 | DLDEWSMATY | L1; L18; L10; L11; |
| | | | | | | L14 |
| 2192 | LPLTQYNRYL | L32 | NTQEVFAQVK | L19; L13 | DLLFNKVTL | L23; L4; L44; L2; |
| | | | | | | L25 |
| 2193 | LPNDDTLRVEAF | L31 | NTWCIRCL | L55; L70 | DPNFKDQVI | L32; L47; L49; |
| | | | | | | L23; L39 |
| 2194 | LPNNTASWFTA | L50 | NVAKSEFDR | L19; L17 | DPSKPSKRSF | L39; L49; L32; |
| | | | | | | L23; L22 |
| 2195 | LPNNTASWFTAL | L56 | NVANYQKV | L24; L20 | DRYPANSIV | L25; L35; L28; |
| | | | | | | L64; L47 |
| 2196 | LPQGTTLPKGF | L39 | NVATLQAEN | L19; L20 | DTGVEHVTF | L53; L54; L10; |
| | | | | | | L52; L11 |
| 2197 | LPQLEQPY | L31 | NVLYENQKL | L25; L24 | DTVSALVY | L11; L1; L29; L10; |
| | | | | | | L18 |
| 2198 | LPQNAVVKIY | L31 | NVNKGEDIQL | L32; L56 | DVRETMSY | L11; L10; L29; |
| | | | | | | L43; L26 |
| 2199 | LPSLATVAYF | L31 | NVNRFNVAI | L20; L32 | DVVECLKL | L25; L11; L23; |
| | | | | | | L10; L74 |
| 2200 | LPSYAAFAT | L50 | NVNRFNVAITR | L19; L17 | DYGDAVVY | L66; L72; L65; |
| | | | | | | L71; L29 |
| 2201 | LPTEVLTEEVV | L50 | NVSLDNVL | L67; L25 | DYKHWPQIAQF | L9; L11; L8; L17; |
| | | | | | | L01 |
| 2202 | LPTEVLTEEVVL | L31 | NVSLVKPSFY | L12; L14 | DYKHYTPSF | L9; L66; L8; L72; |
| | | | | | | L71 |
| 2203 | LPTGVHAGT | L56 | NVTQAFGR | L17; L19 | DYLVSTQEF | L9; L8; L72; L71; |
| | | | | | | L12 |
| 2204 | LPTGVHAGTD | L49 | NVTQAFGRR | L19; L17 | EAKKVKPTV | L23; L20; L70; |
| | | | | | | L58; L47 |
| 2205 | LPTQTVDSS | L50 | NVVTTKIALK | L19; L7 | ECSNLLLQY | L11; L41; L12; |
| | | | | | | L40; L10 |
| 2206 | LPVLQVRD | L50 | NVYIKNAD | L23; L25 | EEIAIILA | L42; L46; L38; |
| | | | | | | L29; L37 |
| 2207 | LPVLQVRDVLV | L50 | NYEDLLIRK | L17; L8 | EETGTLIV | L45; L48; L42; |
| | | | | | | L37; L29 |
| 2208 | LPVNVAFELWA | L50 | NYFITDAQ | L71; L72 | EETGTLIVN | L42; L41; L40; |
| | | | | | | L29; L46 |
| 2209 | LPVSMTKTSV | L50 | NYFLCWHTNCY | L72; L71 | EEVGHTDLM | L29; L41; L36; |
| | | | | | | L45; L42 |
| 2210 | LPYGANKD | L47 | NYGDSATLP | L9; L8 | EEVQELYSP | L42; L46; L38; |
| | | | | | | L41; L40 |
| 2211 | LPYGANKDGI | L47 | NYIAQVDV | L71; L72 | EFSSNVANY | L12; L17; L11; |
| | | | | | | L14; L72 |
| 2212 | LQCIMLVY | L26 | NYIAQVDVV | L72; L71 | EILPVSMTK | L19; L7; L6; L17; |
| | | | | | | L13 |
| 2213 | LQFAYANR | L21 | NYLGKPREQI | L8; L9 | ELNGGAYTR | L17; L21; L19; |
| | | | | | | L15; L11 |
| 2214 | LQFAYANRNR | L21 | NYLKSPNFS | L9; L8 | ELVIGAVIL | L20; L28; L23; |
| | | | | | | L74; L11 |
| 2215 | LQGPPGTGK | L6 | NYLKSPNFSKL | L8; L9 | EQDKNTQEV | L24; L35; L48; |
| | | | | | | L34; L63 |
| 2216 | LQHRLYECLY | L14 | NYLYRLFRK | L17; L8 | ETIQITISS | L20; L11; L19; |
| | | | | | | L10; L17 |
| 2217 | LQKAAITI | L48 | NYMPYFFTLLL | L9; L8 | ETKDVVECL | L20; L10; L11; |
| | | | | | | L13; L70 |
| 2218 | LQLGFSTGV | L24 | NYNYLYRLFR | L17; L15 | EVITFDNLK | L19; L11; L7; L20; |
| | | | | | | L17 |
| 2219 | LQNNELSPV | L24 | NYQHEETI | L71; L72 | EVKPFITES | L11; L10; L20; |
| | | | | | | L17; L43 |
| 2220 | LQPEEEQEE | L24 | NYQHEETIYNL | L8; L9 | EVLTEEVVLK | L19; L7; L17; L6; |
| | | | | | | L13 |
| 2221 | LQPELDSF | L26 | NYQVNGYPN | L71; L72 | EVRTIKVF | L10; L23; L11; |
| | | | | | | L29; L26 |
| 2222 | LQPRTFLLK | L6 | NYSGVVTTVMF | L9; L8 | EYSHVVAF | L9; L71; L72; L62; |
| | | | | | | L66 |
| 2223 | LQQIELKF | L26 | PALQDAYYR | L17; L15 | FAAYSRYRI | L70; L67; L69; |
| | | | | | | L47; L49 |
| 2224 | LQSLQTYV | L48 | PDDQIGYY | L1; L18 | FASFYYVW | L67; L70; L69; |
| | | | | | | L59; L54 |
| 2225 | LQTYVTQQLI | L24 | PELDSFKEEL | L36; L41 | FAVSKGFFK | L60; L19; L61; L7; |
| | | | | | | L58 |
| 2226 | LQVRDVLV | L48 | PETNILLNV | L45; L37 | FELWAKRNI | L45; L41; L37; |
| | | | | | | L40; L36 |
| 2227 | LQYGSFCTQ | L26 | PFAMGIIAM | L72; L71 | FEYVSQPF | L29; L33; L43; |
| | | | | | | L59; L71 |
| 2228 | LQYIRKLH | L48 | PFHPLADNKF | L9; L8 | FFKEGSSV | L71; L72; L62; |
| | | | | | | L43; L23 |
| 2229 | LRARSVSPK | L30 | PFLYLYAL | L71; L72 | FFLYENAFL | L72; L71; L62; L9; |
| | | | | | | L8 |
| 2230 | LRARSVSPKLF | L30 | PFMIDVQQW | L9; L8 | FFYVLGLAA | L50; L71; L72; |
| | | | | | | L46; L51 |
| 2231 | LRCGACIRR | L30 | PFNKWGKAR | L17; L15 | FGWLIVGVAL | L67; L68; L44; |
| | | | | | | L61; L31 |
| 2232 | LRDGWEIV | L35 | PFTIYSLLL | L9; L8 | FIAGLIAI | L59; L67; L74; L3; |
| | | | | | | L61 |
| 2233 | LREPMLQSA | L25 | PFVVSTGYHF | L8; L9 | FISNSWLMWL | L60; L59; L58; |
| | | | | | | L61; L74 |
| 2234 | LRIAGHHLGR | L30 | PFWITIAY | L72; L71 | FITLCFTL | L60; L59; L23; |
| | | | | | | L61; L67 |
| 2235 | LRLIDAMMF | L30 | PGCDGGSLY | L14; L18 | FKHLIPLMY | L66; L12; L27; |
| | | | | | | L31; L59 |
| 2236 | LRPDTRYVLM | L66 | PGLPGTIL | L23; L25 | FLCLFLLPSL | L2; L4; L3; L5; L59 |
| 2237 | LRQWLPTGT | L30 | PIFLIVAAI | L20; L3 | FLFVAAIFYL | L2; L4; L5; L3; L74 |
| 2238 | LRTTNGDFLH | L30 | PIYDEPTTT | L43; L3 | FLIGCNYL | L74; L67; L2; L61; |
| | | | | | | L59 |
| 2239 | LRVCVDTVR | L30 | PLCANGQVF | L26; L9 | FLLFLVLI | L4; L74; L2; L47; |
| | | | | | | L23 |
| 2240 | LRVIGHSMQ | L25 | PLLESELVI | L2; L4 | FLMSFTVLCL | L3; L2; L4; L5; L74 |
| 2241 | LSAPTLVPQEH | L52 | PMDSTVKNY | L1; L18 | FLNGSCGSV | L3; L2; L4; L57; L5 |
| 2242 | LSAQTGIAVL | L53 | PPISFPLCA | L50; L51 | FLPRVESAV | L5; L57; L2; L3; L4 |
| 2243 | LSDDAVVCFN | L1 | PPKNSIDAF | L39; L56 | FLRDGWEIV | L3; L43; L2; L4; L5 |
| 2244 | LSDLQDLK | L1 | PPQTSITSA | L51; L50 | FLYLYALVYF | L74; L12; L59; L2; |
| | | | | | | L62 |
| 2245 | LSEISMDNS | L1 | PPTSFGPL | L56; L22 | FMIDVQQWGF | L43; L8; L12; L11; |
| | | | | | | L9 |
| 2246 | LSEQLDFI | L1 | PQNAVVKI | L48; L24 | FNVLFSTVF | L59; L31; L27; |
| | | | | | | L26; L60 |
| 2247 | LSETKCTLK | L1 | PQSAPHGVVF | L26; L27 | FQFCNDPFL | L44; L58; L61; |
| | | | | | | L69; L28 |
| 2248 | LSFELLHA | L50 | PRRNVATL | L66; L65 | FQSAVKRTI | L24; L48; L34; |
| | | | | | | L64; L44 |
| 2249 | LSFELLHAPA | L50 | PRWYFYYL | L65; L66 | FRVQPTESI | L34; L35; L30; |
| | | | | | | L66; L28 |
| 2250 | LSGFKLKD | L65 | PSGTWLTY | L65; L18 | FSNSGSDVLY | L1; L14; L18; L58; |
| | | | | | | L52 |
| 2251 | LSHQSDIEV | L73 | PTSFGPLVRK | L7; L6 | FSSTFNVPM | L70; L61; L58; |
| | | | | | | L69; L59 |
| 2252 | LSHRFYRL | L60 | PVAYRKVLLR | L19; L21 | FTVEKGIYQ | L60; L58; L20; |
| | | | | | | L61; L69 |
| 2253 | LSKSLTEN | L43 | PVLKGVKL | L23; L74 | FVTHSKGLY | L12; L14; L1; L49; |
| | | | | | | L58 |
| 2254 | LSKSLTENK | L13 | PVNVAFELW | L54; L52 | FVTVYSHL | L74; L60; L59; |
| | | | | | | L61; L68 |
| 2255 | LSLIDFYL | L55 | PVPEVKIL | L57; L64 | FVVPGLPGTIL | L60; L67; L74; |
| | | | | | | L31; L61 |
| 2256 | LSLIDFYLC | L52 | PYEDFQENW | L9; L8 | FWITIAYII | L69; L34; L8; L9; |
| | | | | | | L55 |
| 2257 | LSLIDFYLCF | L52 | PYFFTLLLQL | L9; L8 | FYAYLRKHF | L9; L72; L71; L66; |
| | | | | | | L12 |
| 2258 | LSLPVLQVRD | L52 | PYIKWDLLK | L8; L9 | FYEPQIITT | L62; L72; L66; |
| | | | | | | L71; L9 |
| 2259 | LSLQFKRPI | L55 | PYIVGDVV | L71; L72 | FYLITPVHVM | L71; L72; L66; L9; |
| | | | | | | L8 |
| 2260 | LSLREVRTIKVF | L52 | PYPNASFDNF | L9; L8 | FYLTNDVSFL | L72; L71; L9; L62; |
| | | | | | | L8 |
| 2261 | LSPRWYFY | L1 | QAENVTGLF | L49; L63 | FYPPDEDEE | L66; L72; L62; L9; |
| | | | | | | L71 |
| 2262 | LSPRWYFYYL | L57 | QAITVTPEA | L50; L32 | FYYVWKSY | L71; L72; L43; |
| | | | | | | L66; L59 |
| 2263 | LSRVLGLKTL | L52 | QASSRSSSR | L17; L19 | GADPIHSLR | L63; L19; L15; |
| | | | | | | L18; L68 |
| 2264 | LSSYSLFD | L65 | QAVTANVNAL | L67; L61 | GAMDTTSYR | L15; L19; L17; L7; |
| | | | | | | L21 |
| 2265 | LSTDGNKIADKY | L1 | QEAYEQAV | L33; L45 | GCINANQVI | L24; L34; L36; |
| | | | | | | L28; L45 |
| 2266 | LSTDTGVEHVTF | L52 | QEAYEQAVAN | L38; L40 | GDSEVVLKK | L7; L6; L19; L13; |
| | | | | | | L24 |
| 2267 | LSVCLGSLI | L55 | QEEVQELY | L29; L1 | GEFKLASH | L45; L37; L29; |
| | | | | | | L46; L33 |
| 2268 | LSVVNARL | L60 | QEFKPRSQ | L29; L48 | GEQKSILSP | L38; L42; L46; |
| | | | | | | L37; L45 |
| 2269 | LSYEQFKK | L55 | QEGVVDYGA | L42; L38 | GERVRQAL | L37; L25; L33; |
| | | | | | | L45; L36 |
| 2270 | LSYFIASFRLF | L52 | QEGVVDYGARF | L40; L41 | GERVRQALL | L36; L45; L37; |
| | | | | | | L40; L33 |
| 2271 | LTAESHVD | L55 | QEHYVRITGLY | L40; L41 | GETLPTEV | L45; L48; L37; |
| | | | | | | L46; L36 |
| 2272 | LTAFGLVA | L50 | QEILGTVSWN | L41; L40 | GEVITFDNL | L36; L37; L45; |
| | | | | | | L41; L40 |
| 2273 | LTAFGLVAEWF | L52 | QEKNFTTA | L46; L42 | GEVPVSII | L45; L48; L37; |
| | | | | | | L36; L29 |
| 2274 | LTCATTRQV | L70 | QELGKYEQYIK | L40; L41 | GEYSHVVA | L46; L38; L25; |
| | | | W | | | L37; L42 |
| 2275 | LTDEMIAQYT | L1 | QELIRQGTDY | L40; L41 | GFFTYICGF | L72; L12; L9; L71; |
| | | | | | | L8 |
| 2276 | LTDNVYIK | L1 | QELYSPIF | L29; L33 | GFIAGLIAI | L71; L72; L24; L8; |
| | | | | | | L34 |
| 2277 | LTEEVVLK | L1 | QEPKLGSL | L33; L57 | GFIQQKLAL | L72; L71; L8; L23; |
| | | | | | | L37 |
| 2278 | LTESNKKF | L1 | QEPKLGSLV | L45; L36 | GFQPTNGVGY | L14; L12; L72; |
| | | | | | | L26; L71 |
| 2279 | LTFYLTNDV | L20 | QEPKLGSLVV | L42; L36 | GHFAWWTAF | L27; L28; L35; |
| | | | | | | L34; L26 |
| 2280 | LTFYLTNDVSF | L52 | QESPFVMM | L29; L33 | GHYKHITSK | L27; L28; L6; L7; |
| | | | | | | L35 |
| 2281 | LTKEGATT | L55 | QEVFAQVKQIY | L41; L40 | GLAAIMQLF | L12; L9; L26; L16; |
| | | | | | | L24 |
| 2282 | LTKGTLEPEYF | L52 | QEYADVFHLYL | L41; L40 | GLTVLPPLL | L4; L2; L74; L3; L5 |
| 2283 | LTKPYIKWD | L70 | QFAPSASAFF | L9; L12 | GLVEVEKGV | L2; L5; L3; L24; L4 |
| 2284 | LTLDNQDL | L60 | QFDTYNLWNTF | L9; L8 | GLWLDDVVY | L27; L26; L18; |
| | | | | | | L14; L12 |
| 2285 | LTLDNQDLN | L52 | QFKHLIPL | L71; L72 | GPKVKYLYF | L39; L23; L49; |
| | | | | | | L22; L43 |
| 2286 | LTLDNQDLNGN | L52 | QFPNTYLEG | L72; L71 | GQQQQGQTV | L24; L48; L28; |
| | W | | | | | L26; L35 |
| 2287 | LTLGVYDY | L1 | QGAVDINKL | L64; L34 | GRFVLALL | L25; L64; L35; |
| | | | | | | L66; L30 |
| 2288 | LTNDNTSRYW | L52 | QGDDYVYLPY | L18; L1 | GSDNVTDF | L63; L62; L68; |
| | | | | | | L74; L54 |
| 2289 | LTNDVSFLA | L1 | QHEVLLAPL | L34; L28 | GSIIQFPNTY | L26; L14; L55; |
| | | | | | | L52; L27 |
| 2290 | LTNDVSFLAH | L6 | QHEVLLAPLL | L28; L34 | GSKSPIQYI | L55; L13; L24; |
| | | | | | | L70; L53 |
| 2291 | LTPGDSSSG | L57 | QHMVVKAALL | L28; L34 | GSLAATVRL | L44; L53; L73; |
| | | | | | | L74; L60 |
| 2292 | LTPLGIDLDEW | L52 | QIDGYVMHANY | L18; L1 | GSLVVRCSF | L52; L53; L54; |
| | | | | | | L55; L16 |
| 2293 | LTPVVQTIEV | L5 | QIGEYTFEK | L7; L6 | GTDPYEDF | L1; L54; L62; L18; |
| | | | | | | L63 |
| 2294 | LTRPLLES | L59 | QIGGYTEKW | L54; L16 | GTHWFVTQR | L21; L7; L15; L19; |
| | | | | | | L17 |
| 2295 | LTRPLLESE | L52 | QIVESCGNF | L10; L11 | GVAPGTAV | L28; L20; L51; |
| | | | | | | L23; L24 |
| 2296 | LTSMKYFVK | L7 | QLDEEQPM | L63; L62 | GVDAVNLL | L63; L74; L68; |
| | | | | | | L62; L1 |
| 2297 | LTSQWLTNI | L53 | QLDFIDTKRGVY | L1; L18 | GVFCGVDAV | L20; L3; L24; L4; |
| | | | | | | L2 |
| 2298 | LTSQWLTNIF | L52 | QLGIEFLKR | L21; L6 | GVPVVDSYY | L12; L14; L26; |
| | | | | | | L18; L11 |
| 2299 | LTTAAKLM | L55 | QLMCQPILLL | L4; L2 | GVVQLTSQW | L10; L52; L54; |
| | | | | | | L49; L16 |
| 2300 | LTTAAKLMV | L73 | QLNRALTGI | L3; L4 | GVVQQLPETY | L12; L26; L10; |
| | | | | | | L14; L52 |
| 2301 | LTVFFDGRV | L20 | QLPETYFTQ | L5; L57 | GVYSVIYL | L74; L67; L64; |
| | | | | | | L69; L58 |
| 2302 | LTVFFDGRVD | L55 | QLRKQIRSA | L3; L51 | GVYYPDKVFR | L7; L15; L19; L21; |
| | | | | | | L6 |
| 2303 | LVAAGLEAPFLY | IL12 | QLRVIGHSM | L43; L26 | GYINVFAF | L72; L71; L9; L8; |
| | | | | | | L66 |
| 2304 | LVAVPTGYV | L20 | QLTPTWRV | L48; L4 | GYINVFAFPF | L8; L9; L12; L72; |
| | | | | | | L71 |
| 2305 | LVKQGDDY | L26 | QMAPISAMVR | L21; L19 | GYLQPRTFL | L9; L8; L72; L71; |
| | | | | | | L36 |
| 2306 | LVLARKHTT | L23 | QNAQALNTL | L34; L28 | HECFVKRV | L48; L45; L37; |
| | | | | | | L33; L29 |
| 2307 | LVLIMLII | L47 | QNYGDSATL | L28; L65 | HFAIGLALYY | L12; L18; L14; |
| | | | | | | L72; L71 |
| 2308 | LVLIMLIIFW | L52 | QPEEEQEED | L32; L49 | HFDEGNCDTL | L62; L34; L68; |
| | | | | | | L67; L63 |
| 2309 | LVLLPLVSS | L50 | QPIGALDISA | L50; L51 | HFIETISLA | L8; L46; L62; L11; |
| | | | | | | L72 |
| 2310 | LVPFWITI | L47 | QPILLLDQA | L50; L51 | HGFELTSM | L25; L48; L29; |
| | | | | | | L59; L23 |
| 2311 | LVPFWITIAYI | L5 | QPITNCVKM | L32; L22 | HHMELPTGV | L28; L35; L3; L34; |
| | | | | | | L2 |
| 2312 | LVPHVGEI | L47 | QPLEQPTSEA | L50; L51 | HITSKETLY | L12; L14; L11; |
| | | | | | | L18; L58 |
| 2313 | LVPHVGEIPV | L5 | QPSVGPKQASL | L56; L22 | HLLIGLAKR | L21; L15; L17; L6; |
| | | | | | | L11 |
| 2314 | LVPQEHYV | L5 | QPVSELLTPL | L22; L31 | HLLLVAAGL | L44; L4; L2; L5; |
| | | | | | | L23 |
| 2315 | LVPQEHYVRI | L5 | QQQTTLKGV | L48; L24 | HLMSFPQSA | L3; L2; L43; L46; |
| | | | | | | L50 |
| 2316 | LVRKIFVD | L65 | QRNFYEPQI | L34; L30 | HLYLQYIRKL | L3; L64; L24; L4; |
| | | | | | | L44 |
| 2317 | LVSDIDITFLKK | L6 | QRVAGDSGF | L30; L34 | HPDSATLV | L63; L62; L47; |
| | | | | | | L50; L32 |
| 2318 | LVTLAILTAL | L56 | QSADAQSFLNR | L15; L7 | HPLADNKFAL | L32; L56; L31; |
| | | | | | | L23; L39 |
| 2319 | LVYAADPAM | L32 | QSAPHGVV | L70; L48 | HPNPKGFCDL | L32; L39; L56; |
| | | | | | | L22; L49 |
| 2320 | LVYDNKLKAH | L43 | QSAVKRTI | L55; L48 | HPNQEYADV | L51; L50; L32; |
| | | | | | | L49; L39 |
| 2321 | LVYDNKLKAHK | L6 | QSDIEVTGD | L65; L1 | HSKGLYRKC | L55; L13; L14; |
| | | | | | | L52; L10 |
| 2322 | LVYFLQSINF | L12 | QSFLNRVC | L55; L48 | HSYFTSDY | L18; L43; L14; |
| | | | | | | L26; L1 |
| 2323 | LWCKDGHVETF | L8 | QSYGFQPTN | L48; L55 | HTDFSSEI | L68; L63; L62; |
| | | | | | | L18; L1 |
| 2324 | LWLLWPVTLA | L50 | QTLEILDI | L55; L48 | HTDESSEII | L68; L34; L18; |
| | | | | | | L63; L1 |
| 2325 | LWNTFTRL | L62 | QTMLFTMLRK | L7; L6 | HTDLMAAYV | L62; L63; L18; L1; |
| | | | | | | L68 |
| 2326 | LYALVYFLQ | L9 | QTSITSAVL | L28; L53 | HVSGTNGTKR | L19; L21; L17; |
| | | | | | | L11; L15 |
| 2327 | LYDKLQFT | L62 | QTTETAHSC | L55; L54 | HVVAFNTLLF | L11; L12; L10; |
| | | | | | | L49; L52 |
| 2328 | LYDKLVSS | L62 | QTTTIQTI | L55; L48 | HWPQIAQF | L62; L33; L9; L11; |
| | | | | | | L71 |
| 2329 | LYIDINGN | L72 | QTVDSSQGSEY | L14; L11 | HYVRITGLY | L14; L12; L11; |
| | | | | | | L49; L66 |
| 2330 | LYIDINGNLHP | L8 | QVAKSHNIAL | L56; L20 | IAANTVIW | L54; L69; L52; |
| | | | | | | L49; L70 |
| 2331 | LYKLMGHF | L9 | QVEQKIAEI | L20; L10 | IAATRGATV | L58; L70; L47; |
| | | | | | | L61; L69 |
| 2332 | LYKMQRML | L71 | QVIVNNLDK | L7; L6 | IAEIPKEEV | L63; L68; L67; |
| | | | | | | L49; L47 |
| 2333 | LYPTLNISDEF | L9 | QVNGLTSIK | L6; L7 | IAIVMVTIML | L74; L60; L61; |
| | | | | | | L67; L31 |
| 2334 | LYSPIFLIVAA | L50 | QVNGYPNMF | L16; L58 | IANQFNSAI | L68; L47; L32; |
| | | | | | | L70; L57 |
| 2335 | MADLVYALRH | L68 | QVNGYPNMFIT | L21; L17 | IAQYTSALL | L60; L61; L63; |
| | | | R | | | L32; L74 |
| 2336 | MADQAMTQ | L68 | QVVDADSKI | L10; L20 | IAYIICIST | L43; L69; L67; |
| | | | | | | L47; L50 |
| 2337 | MAFPSGKVEG | L31 | QWMVMFTPL | L71; L72 | IFGTVYEKL | L8; L9; L62; L72; |
| | | | | | | L71 |
| 2338 | MAGNGGDAA | L50 | QWNLVIGFL | L62; L40 | IFVDGVPF | L72; L71; L62; L9; |
| | | | | | | L8 |
| 2339 | MAGNGGDAAL | L32 | QYIKWPWY | L71; L72 | IFWFSLEL | L72; L62; L71; |
| | | | | | | L68; L67 |
| 2340 | MAPISAMVR | L57 | QYIKWPWYIW | L9; L8 | IFYLITPV | L48; L72; L71; |
| | | | | | | L47; L62 |
| 2341 | MASLVLAR | L17 | QYIKWPWYIWL | L8; L9 | IGAGICASY | L14; L26; L12; |
| | | | | | | L43; L27 |
| 2342 | MASLVLARK | L19 | QYIRKLHDEL | L9; L8 | IIIWFLLLSV | L3; L73; L4; L5; |
| | | | | | | L20 |
| 2343 | MAYITGGVVQ | L49 | QYLNTLTLA | L8; L9 | IIKTIQPRV | L58; L48; L5; L13; |
| | | | | | | L3 |
| 2344 | MAYITGGVVQL | L69 | QYVFCTVNAL | L72; L71 | IIRENNRVV | L3; L13; L58; L39; |
| | | | | | | L48 |
| 2345 | MDGSIIQF | L33 | RAFDIYNDKV | L24; L20 | IKDFGGFNF | L27; L34; L68; |
| | | | | | | L63; L9 |
| 2346 | MDLFMRIF | L33 | RAGEAANF | L54; L53 | ILDGISQYSL | L5; L68; L63; L2; |
| | | | | | | L67 |
| 2347 | MDNLACEDL | L33 | RAMPNMLR | L15; L21 | ILGLPTQTV | L2; L24; L5; L4; L3 |
| 2348 | MDSTVKNYF | L33 | RARSVASQSI | L55; L53 | ILGTVSWNL | L2; L4; L5; L74; L9 |
| 2349 | MEKLKTLVAT | L42 | RARTVAGVSI | L53; L44 | ILRKGGRTI | L55; L3; L24; L39; |
| | | | | | | L48 |
| 2350 | MELPTGVHAG | L40 | RARVECFDKF | L52; L53 | ILTSLLVL | L74; L23; L59; L4; |
| | | | | | | L73 |
| 2351 | MELTPVVQ | L29 | RATRVECTTI | L54; L53 | IMASLVLAR | L19; L17; L6; L21; |
| | | | | | | L15 |
| 2352 | MESEFRVYSS | L42 | RCNLGGAVCR | L21; L15 | INPTDQSSY | L14; L12; L43; |
| | | | | | | L72; L71 |
| 2353 | MFDAYVNT | L62 | RDLPQGFSA | L51; L46 | IPFAMQMAY | L31; L49; L12; |
| | | | | | | L50; L71 |
| 2354 | MFLGTCRR | L17 | RDLPQGFSAL | L33; L37 | IPTITQMNL | L56; L39; L32; |
| | | | | | | L22; L49 |
| 2355 | MFTPLIQPI | L9 | RDPQTLEIL | L33; L57 | IQYGRSGETL | L25; L27; L26; |
| | | | | | | L28; L44 |
| 2356 | MFTPLVPFWI | L9 | REFVFKNIDGY | L29; L41 | ITIAYIICI | L20; L69; L70; |
| | | | | | | L73; L53 |
| 2357 | MGIIAMSA | L50 | REGYLNSTN | L45; L36 | ITLKKRWQL | L53; L16; L73; |
| | | | | | | L60; L4 |
| 2358 | MGIIAMSAF | L59 | RELNGGAYTRY | L40; L41 | IVDSVTVKN | L63; L5; L68; L49; |
| | | | | | | L18 |
| 2359 | MHAASGNL | L28 | REQIDGYVMHA | L42; L38 | IWLGFIAGL | L4; L8; L9; L30; |
| | | | | | | L62 |
| 2360 | MHHMELPTGV | L28 | RETMSYLF | L33; L29 | IYDEPTTTT | L62; L68; L9; L8; |
| | | | | | | L36 |
| 2361 | MISAGFSLWVY | L14 | REVLSDRELHL | L36; L40 | KAFKQIVES | L69; L53; L70; |
| | | | | | | L73; L58 |
| 2362 | MLAHAEETR | L21 | RFASVYAW | L54; L71 | KAGGTTEM | L70; L58; L54; |
| | | | | | | L59; L63 |
| 2363 | MLDNRATLQ | L1 | RFDTRVLSN | L18; L62 | KAIVSTIQR | L21; L15; L19; |
| | | | | | | L17; L7 |
| 2364 | MLDNRATLQAI | L5 | RFQTLLAL | L72; L71 | KCRSKNPLLY | L13; L18; L6; L12; |
| | | | | | | L14 |
| 2365 | MLFTMLRK | L6 | RFQTLLALH | L14; L15 | KDCVVLHSY | L14; L16; L18; |
| | | | | | | L26; L33 |
| 2366 | MLKTVYSD | L23 | RFVSLAIDAY | L14; L12 | KDLPKEITV | L33; L73; L2; L37; |
| | | | | | | L4 |
| 2367 | MLKTVYSDV | L3 | RFYFYTSK | L71; L72 | KDLSPRWYF | L33; L16; L8; L52; |
| | | | | | | L9 |
| 2368 | MLLEIKDTEK | L6 | RGGSQASSR | L15; L21 | KEIDRLNEVA | L38; L46; L42; |
| | | | | | | L36; L37 |
| 2369 | MLLEIKDTEKY | L12 | RHSLSHFV | L33; L28 | KELLQNGM | L33; L37; L36; |
| | | | | | | L45; L29 |
| 2370 | MLLEKCDL | L23 | RIDGDMVPHI | L5; L63 | KELLVYAA | L33; L38; L46; |
| | | | | | | L42; L37 |
| 2371 | MLRIMASL | L23 | RIIMRLWLCW | L54; L16 | KELLVYAAD | L38; L33; L46; |
| | | | | | | L40; L42 |
| 2372 | MLWCKDGHV | L2 | RIIPARAR | L21; L15 | KESVQTFFKL | L36; L37; L41; |
| | | | | | | L40; L45 |
| 2373 | MMFVKHKH | L48 | RIKIVQML | L23; L39 | KFLTENLL | L71; L8; L62; L72; |
| | | | | | | L9 |
| 2374 | MMFVKHKHAF | L16 | RILGAGCFV | L73; L13 | KFNGLTVL | L62; L71; L72; L8; |
| | | | | | | L56 |
| 2375 | MMISAGFSLW | L10 | RIMASLVLARK | L6; L7 | KFTDGVCL | L72; L71; L62; L8; |
| | | | | | | L56 |
| 2376 | MNLKYAISA | L50 | RIQPGQTFSV | L16; L4 | KFYGGWHNML | L71; L8; L72; L9; |
| | | | | | | L16 |
| 2377 | MPASWVMRIMT | L49 | RIRSVYPVA | L51; L13 | KGFCKLHNW | L16; L54; L52; |
| | W | | | | | L49; L53 |
| 2378 | MPILTLTRALTA | L50 | RKHFSMMIL | L44; L27 | KGIYQTSNF | L54; L27; L8; L26; |
| | | | | | | L16 |
| 2379 | MPLKAPKE | L47 | RKHTTCCSL | L44; L27 | KGPKVKYLY | L14; L12; L66; |
| | | | | | | L54; L18 |
| 2380 | MPLKAPKEII | L47 | RKIFVDGVPF | L27; L44 | KHAFLCLFL | L28; L34; L73; |
| | | | | | | L44; L35 |
| 2381 | MPLKAPKEIIF | L31 | RKLDNDAL | L44; L27 | KHTPINLV | L28; L48; L73; |
| | | | | | | L64; L35 |
| 2382 | MPLSAPTLVPQ | L50 | RKVQHMVVK | L44; L27 | KIADKYVRNL | L3; L16; L4; L13; L |
| | | | | | | 64 |
| 2383 | MPNLYKMQR | L17 | RKYKGIKIQ | L27; L44 | KIILFLALI | L73; L3; L48; L24; |
| | | | | | | L16 |
| 2384 | MPNLYKMQRML | L39 | RLIIRENNR | L21; L15 | KLDGFMGRI | L5; L18; L63; L2; |
| | | | | | | L24 |
| 2385 | MPNMLRIMAS | L50 | RLIIRENNRV | L24; L2 | KLGSLVVRC | L4; L2; L16; L21; |
| | | | | | | L42 |
| 2386 | MPNMLRIMASL | L22 | RLISMMGFK | L6; L13 | KLKDCVMY | L26; L13; L43; |
| | | | | | | L14; L18 |
| 2387 | MPVCVETKAI | L32 | RLITGRLQS | L16; L13 | KLLEQWNL | L2; L4; L55; L16; |
| | | | | | | L5 |
| 2388 | MQNCVLKLKV | L24 | RLKLFAAETL | L16; L26 | KLNEEIAIIL | L16; L2; L4; L5; |
| | | | | | | L44 |
| 2389 | MQRKLEKM | L48 | RLKLFDRYFKY | L14; L18 | KLVSSFLEM | L16; L5; L73; L6; |
| | | | | | | L27 |
| 2390 | MQTMLFTML | L44 | RLQAGNATEV | L2; L24 | KMKDLSPRWY | L14; L13; L55; |
| | | | | | | L16; L26 |
| 2391 | MQVESDDY | L26 | RLWLCWKCR | L21; L15 | KNFKSVLY | L27; L54; L65; |
| | | | | | | L18; L26 |
| 2392 | MQVESDDYI | L34 | RLYLDAYNM | L21; L16 | KNGSIHLY | L14; L65; L18; |
| | | | | | | L55; L54 |
| 2393 | MRELNGGAY | L1 | RNAGIVGV | L24; L33 | KPCIKVATV | L51; L39; L47; |
| | | | | | | L23; L22 |
| 2394 | MRLWLCWKC | L30 | RNARNGVLI | L53; L24 | KPFERDIST | L56; L51; L39; |
| | | | | | | L32; L50 |
| 2395 | MRLWLCWKCR | L30 | RNLQEFKPR | L15; L21 | KPFLNKVV | L47; L39; L51; |
| | | | | | | L25; L50 |
| 2396 | MRTFKVSI | L35 | RNRFLYIIKL | L13; L44 | KPNELSRV | L51; L39; L56; |
| | | | | | | L47; L48 |
| 2397 | MRTFKVSIWNL | L30 | RNTNPIQL | L73; L56 | KPYIKWDLL | L23; L39; L56; |
| | | | | | | L22; L47 |
| 2398 | MRVIHFGA | L25 | RNYIAQVD | L65; L55 | KQGDDYVY | L26; L14; L65; |
| | | | | | | L27; L66 |
| 2399 | MSALNHTKKW | L52 | RNYIAQVDV | L73; L24 | KQLIKVTLV | L48; L24; L73; |
| | | | | | | L33; L4 |
| 2400 | MSALNHTKKWK | L1 | RNYVFTGYR | L15; L21 | KQLIKVTLVF | L16; L27; L26; L8; |
| | Y | | | | | L52 |
| 2401 | MSDNGPQNQ | L1 | RPQGLPNNTA | L51; L22 | KQLPFFYY | L27; L14; L26; |
| | | | | | | L12; L16 |
| 2402 | MSDRDLYD | L1 | RPQIGVVRE | L56; L39 | KRFDNPVLPF | L30; L66; L65; |
| | | | | | | L16; L6 |
| 2403 | MSDRDLYDK | L1 | RQALLKTVQ | L44; L24 | KRFKESPFEL | L65; L66; L30; |
| | | | | | | L35; L16 |
| 2404 | MSDRDLYDKL | L1 | RQLLFVVE | L27; L44 | KSAFVNLKQL | L55; L53; L73; |
| | | | | | | L13; L44 |
| 2405 | MSDVKCTSVVL | L68 | RQLLFVVEVV | L24; L48 | KSAGFPFNKW | L52; L54; L16; |
| | | | | | | L53; L18 |
| 2406 | MSLSEQLR | L17 | RQVVNVVTTK | L30; L7 | KSASVYYSQL | L55; L53; L44; |
| | | | | | | L54; L73 |
| 2407 | MSRIGMEV | L48 | RRAFGEYSH | L30; L65 | KSDGTGTI | L55; L68; L63; |
| | | | | | | L54; L18 |
| 2408 | MSYEDQDALFAY | L1 | RRATCFSTA | L65; L30 | KSFTVEKGI | L55; L53; L54; |
| | | | | | | L52; L16 |
| 2409 | MSYLFQHA | L50 | RRCPAEIVD | L65; L30 | KSHNIALI | L55; L48; L73; |
| | | | | | | L53; L54 |
| 2410 | MSYLFQHAN | L48 | RRGPEQTQG | L65; L30 | KSILSPLYAF | L52; L53; L54; |
| | | | | | | L16; L26 |
| 2411 | MTYRRLISMM | L11 | RRLISMMGF | L30; L33 | KSLNVAKSEF | L53; L52; L54; |
| | | | | | | L16; L55 |
| 2412 | MVDTSLSG | L68 | RRSFYVYAN | L65; L30 | KSPNFSKL | L57; L73; L63; |
| | | | | | | L55; L54 |
| 2413 | MVDTSLSGFKL | L68 | RRVWTLMNV | L30; L64 | KSPNFSKLI | L55; L64; L53; |
| | | | | | | L57; L73 |
| 2414 | MVLGSLAATV | L20 | RRVWTLMNVL | L65; L30 | KSQDLSVV | L73; L70; L48; |
| | | | | | | L69; L18 |
| 2415 | MVMCGGSLYVK | L7 | RSDVLLPLT | L18; L1 | KSREETGLLM | L52; L13; L53; |
| | | | | | | L18; L55 |
| 2416 | MVMFTPLV | L48 | RSEKSYELQ | L18; L1 | KSWMESEFRVY | L14; L18; L52; |
| | | | | | | L55; L16 |
| 2417 | MVMFTPLVP | L50 | RSFIEDLLFNK | L7; L6 | KTDGTLMIERF | L18; L54; L52; |
| | | | | | | L53; L16 |
| 2418 | MVRMYIFFA | L50 | RSGARSKQR | L21; L15 | KTHVQLSL | L73; L53; L55; |
| | | | | | | L54; L60 |
| 2419 | MVVKAALL | L74 | RSKNPLLYD | L13; L15 | KTLATHGL | L54; L55; L16; |
| | | | | | | L33; L53 |
| 2420 | MWKGYGCSC | L43 | RSLPGVFC | L55; L54 | KTPPIKDF | L53; L54; L52; |
| | | | | | | L55; L66 |
| 2421 | MWLSYFIA | L50 | RSLPGVFCGV | L13; L73 | KTYERHSLSHF | L16; L53; L54; |
| | | | | | | L11; L52 |
| 2422 | MYDPKTKN | L62 | RSSVLHST | L55; L54 | KVFRSSVL | L54; L53; L56; |
| | | | | | | L44; L55 |
| 2423 | MYLKLRSDVL | L9 | RSVASQSII | L55; L54 | KVGGNYNYLY | L14; L18; L16; |
| | | | | | | L12; L54 |
| 2424 | MYTPHTVLQ | L9 | RSVASQSIIAY | L18; L14 | KVKYLYFIK | L13; L7; L6; L21; |
| | | | | | | L15 |
| 2425 | NAAIVLQLP | L49 | RSVYPVASP | L65; L54 | KVTLVFLFV | L73; L13; L20; |
| | | | | | | L24; L5 |
| 2426 | NAANVYLK | L19 | RTAPHGHV | L73; L18 | KWADNNCYL | L44; L8; L9; L73; |
| | | | | | | L36 |
| 2427 | NADLYKLMG | L68 | RTAPHGHVMVE | L16; L52 | KWDLLKYDF | L62; L53; L9; L63; |
| | | | | | | L33 |
| 2428 | NALPETTA | L50 | RTCCLCDRR | L21; L15 | KYKYFSGAM | L66; L71; L72; |
| | | | | | | L13; L14 |
| 2429 | NANQVIVNNL | L31 | RTFKVSIWNLDY | L18; L14 | KYTMADLVY | L14; L66; L72; |
| | | | | | | L71; L12 |
| 2430 | NAPGCDVTD | L49 | RTIAFGGCV | L73; L13 | LADAGFIKQ | L63; L49; L68; |
| | | | | | | L32; L18 |
| 2431 | NAPGCDVTDV | L57 | RTIKVFTT | L55; L54 | LADAGFIKQY | L18; L1; L49; L70; |
| | | | | | | L31 |
| 2432 | NAQALNTLV | L70 | RTLETAQNS | L53; L54 | LAFLLFLVLI | L47; L50; L69; |
| | | | | | | L31; L52 |
| 2433 | NARNGVLI | L47 | RTLETAQNSVR | L21; L15 | LAKKFDTF | L58; L59; L23; |
| | | | | | | L47; L52 |
| 2434 | NARNGVLIT | L69 | RTNVYLAV | L73; L18 | LALHRSYL | L60; L61; L59; |
| | | | | | | L47; L74 |
| 2435 | NATNKATY | L70 | RTQLPPAY | L18; L14 | LALITLATC | L47; L50; L59; |
| | | | | | | L52; L31 |
| 2436 | NATNKATYK | L19 | RTVAGVSIC | L54; L16 | LATCELYHY | L31; L49; L12; |
| | | | | | | L52; L58 |
| 2437 | NATRFASV | L47 | RTVYDDGAR | L21; L15 | LAYILFTRFF | L52; L49; L47; |
| | | | | | | L60; L31 |
| 2438 | NCDTLKEI | L68 | RTVYDDGARR | L15; L21 | LCLFLLPSL | L4; L31; L50; L59; |
| | | | | | | L47 |
| 2439 | NCDVVIGIV | L62 | RVAGDSGF | L54; L26 | LEEKFKEGV | L45; L46; L42; |
| | | | | | | L38; L37 |
| 2440 | NCVADYSVLY | L31 | RVCGVSAARL | L56; L14 | LEFGATSA | L46; L25; L38; |
| | | | | | | L29; L37 |
| 2441 | NCVLKLKV | L25 | RVCVDTVR | L21; L15 | LEQPTSEAV | L38; L45; L42; |
| | | | | | | L46; L36 |
| 2442 | NDFNLVAMK | L19 | RVIGHSMQN | L13; L11 | LEQYVFCTV | L45; L38; L42; |
| | | | | | | L37; L46 |
| 2443 | NDLCFTNV | L33 | RVIHFGAGSDK | L6; L7 | LFCLLNRYF | L62; L72; L9; L71; |
| | | | | | | L12 |
| 2444 | NDLCFTNVY | L29 | RVLQKAAITI | L16; L53 | LFDESGEFKL | L62; L68; L63; L5; |
| | | | | | | L8 |
| 2445 | NDPFLGVYY | L12 | RVVFVLWAH | L7; L6 | LFFSYFAVHF | L71; L9; L72; L12; |
| | | | | | | L8 |
| 2446 | NDSKEGFF | L33 | RVVFVLWAHGF | L16; L52 | LFVAAIFYL | L72; L62; L71; L9; |
| | | | | | | L8 |
| 2447 | NDSKEGFFTY | L29 | RVYANLGERVR | L21; L15 | LFVTVYSHL | L72; L8; L71; L62; |
| | | | | | | L9 |
| 2448 | NEFACVVADA | L46 | RYFRLTLGVY | L14; L12 | LHDELTGHM | L62; L63; L28; L1; |
| | | | | | | L5 |
| 2449 | NEFYAYLR | L29 | RYLALYNKYKY | L14; L12 | LHPDSATLV | L5; L28; L64; L57; |
| | | | | | | L34 |
| 2450 | NEKQEILGT | L42 | RYMSALNH | L71; L72 | LIDFYLCF | L68; L1; L62; L63; |
| | | | | | | L60 |
| 2451 | NEKTHVQLS | L29 | RYMSALNHT | L9; L8 | LIDSYFVVK | L7; L6; L5; L68; L1 |
| 2452 | NENGTITD | L29 | RYPANSIV | L71; L72 | LIIFWFSL | L23; L60; L61; |
| | | | | | | L59; L67 |
| 2453 | NESLIDLQ | L29 | RYPANSIVCR | L15; L21 | LIKVTLVF | L59; L43; L26; |
| | | | | | | L23; L58 |
| 2454 | NETLVTMP | L29 | RYPANSIVCRF | L8; L9 | LINIIIWF | L59; L26; L74; |
| | | | | | | L23; L43 |
| 2455 | NEVAKNLN | L29 | RYVLMDGSIIQF | L9; L8 | LIQPIGAL | L61; L59; L60; |
| | | | | | | L23; L74 |
| 2456 | NEYRLYLD | L29 | SAAKKNNLPF | L58; L43 | LLEDEFTPF | L5; L1; L26; L12 |
| | | | | | | L9 |
| 2457 | NFGDQELIR | L17 | SADAQSFLN | L68; L63 | LLFNKVTLA | L3; L2; L4; L6; L5 |
| 2458 | NFVRIIMR | L17 | SADAQSFLNR | L7; L19 | LLIIMRTF | L26; L55; L59; |
| | | | | | | L43; L23 |
| 2459 | NFYEPQIITT | L71 | SAINRPQI | L70; L47 | LLLLFVTV | L47; L23; L50; L4; |
| | | | | | | L2 |
| 2460 | NGDRWFLNR | L17 | SAINRPQIG | L69; L70 | LLMPILTLT | L2; L5; L3; L4; L6 |
| 2461 | NGGAYTRYV | L64 | SAINRPQIGV | L20; L58 | LLTDEMIAQY | L11; L14; L12; |
| | | | | | | L26; L18 |
| 2462 | NGLTGTGV | L25 | SALNHTKKWKY | L12; L14 | LLYDANYF | L59; L62; L26; |
| | | | | | | L74; L72 |
| 2463 | NGMNGRTIL | L25 | SAMVRMYIFF | L58; L43 | LNEEIAIIL | L32; L31; L63; |
| | | | | | | L68; L62 |
| 2464 | NGNKGAGGHSY | L14 | SAQTGIAV | L70; L23 | LPAPRTLLT | L22; L47; L31; |
| | | | | | | L51; L49 |
| 2465 | NGQVFGLY | L14 | SASAFFGMSR | L19; L17 | LPFFSNVTWF | L31; L49; L32; |
| | | | | | | L39; L47 |
| 2466 | NGSPSGVY | L14 | SASKIITLKK | L7; L19 | LPGCDGGSL | L39; L22; L32; |
| | | | | | | L56; L31 |
| 2467 | NGYLTSSS | L25 | SAVGNICYT | L69; L58 | LPKEITVA | L50; L51; L23; |
| | | | | | | L39; L47 |
| 2468 | NGYTVEEA | L25 | SAVKLQNNEL | L67; L32 | LPNNTASWF | L49; L31; L32; |
| | | | | | | L39; L22 |
| 2469 | NHNFLVQAG | L35 | SAVLQSGFRK | L7; L19 | LQAGNATEV | L24; L58; L28; L3; |
| | | | | | | L64 |
| 2470 | NHTKKWKY | L29 | SDFVRATAT | L33; L25 | LQAVGACVL | L44; L28; L61; |
| | | | | | | L35; L25 |
| 2471 | NICYTPSKL | L63 | SDIDYVPL | L43; L71 | LRVEAFEYY | L66; L30; L31; |
| | | | | | | L12; L65 |
| 2472 | NIFGTVYEKLK | L19 | SDNIALLV | L33; L48 | LSDDAVVC | L68; L63; L1; L62; |
| | | | | | | L18 |
| 2473 | NIGEQKSIL | L67 | SDRELHLSW | L49; L52 | LSLPVLQV | L73; L48; L47; |
| | | | | | | L70; L59 |
| 2474 | NIIIWFLL | L23 | SDVENPHLM | L29; L33 | LSSTASAL | L57; L61; L60; |
| | | | | | | L59; L55 |
| 2475 | NIIRGWIF | L29 | SDYDYYRYN | L65; L43 | LSVVNARLR | L55; L19; L15; |
| | | | | | | L52; L17 |
| 2476 | NIKFADDL | L23 | SEAARVVR | L29; L42 | LSYGIATVR | L19; L15; L17; |
| | | | | | | L21; L69 |
| 2477 | NIKPVPEVK | L13 | SEAARVVRSIF | L41; L40 | LTAVVIPTK | L7; L13; L6; L52; |
| | | | | | | L19 |
| 2478 | NINLHTQVV | L73 | SEDAQGMDNL | L36; L37 | LTLGVYDYL | L67; L60; L53; |
| | | | | | | L61; L73 |
| 2479 | NITRFQTLL | L74 | SEDKRAKVTSA | L38; L42 | LVFLGIITTV | L3; L20; L50; L51; |
| | | | | | | L47 |
| 2480 | NIVNVSLV | L48 | SEDNQTTT | L45; L37 | LVQSTQWSL | L32; L39; L60; |
| | | | | | | L56; L4 |
| 2481 | NKDGIIWVA | L35 | SEETGTLIVN | L36; L42 | LVYALRHF | L59; L55; L47; |
| | | | | | | L52; L58 |
| 2482 | NKFALTCF | L27 | SEEVVENP | L42; L38 | LVYDNKLKA | L50; L46; L51; |
| | | | | | | L24; L3 |
| 2483 | NKGEDIQL | L66 | SEFSSLPSYAA | L46; L42 | LYCIDGALL | L9; L62; L8; L72; |
| | | | | | | L71 |
| 2484 | NKHADFDTW | L49 | SEFSSLPSYAAF | L41; L40 | LYLGGMSY | L71; L72; L66; |
| | | | | | | L12; L14 |
| 2485 | NKHIDAYKTF | L27 | SEGLNDNLLEI | L41; L45 | LYLGGMSYY | L71; L72; L12; |
| | | | | | | L14; L8 |
| 2486 | NKKDWYDF | L66 | SEMVMCGGSLY | L40; L41 | LYLYALVY | L72; L71; L65; |
| | | | | | | L12; L66 |
| 2487 | NKKFLPFQQF | L27 | SEQLRKQIRSA | L42; L38 | LYQPPQTSI | L9; L8; L72; L71; |
| | | | | | | L28 |
| 2488 | NKWGKARLYY | L27 | SEVGPEHSLAEY | L41; L40 | LYVNKHAF | L72; L71; L9; L8; |
| | | | | | | L66 |
| 2489 | NKYKYFSGA | L46 | SEVVLKKLK | L41; L40 | MACLVGLMW | L49; L31; L54; |
| | | | | | | L50; L52 |
| 2490 | NLAWPLIV | L3 | SEYKGPITDVF | L29; L41 | MADLVYALR | L19; L68; L17; |
| | | | | | | L63; L1 |
| 2491 | NLAWPLIVTAL | L4 | SFCTQLNR | L17; L15 | MCQPILLL | L74; L28; L73; |
| | | | | | | L25; L61 |
| 2492 | NLDKSAGFP | L1 | SFCTQLNRAL | L71; L72 | MELPTGVHA | L46; L42; L38; |
| | | | | | | L50; L40 |
| 2493 | NLDSCKRV | L63 | SFGGASCCLY | L14; L12 | MELTPVVQT | L42; L29; L38; |
| | | | | | | L46; L33 |
| 2494 | NLDSCKRVLNV | L5 | SFLAHIQW | L71; L72 | MESEFRVY | L29; L40; L46; |
| | | | | | | L45; L41 |
| 2495 | NLDYIINLII | L68 | SFLGRYMSAL | L71; L72 | MFTPLVPF | L72; L62; L71; L9; |
| | | | | | | L59 |
| 2496 | NLHPDSATLV | L2 | SFLNGFAV | L71; L72 | MFTPLVPFW | L9; L49; L54; L52; |
| | | | | | | L8 |
| 2497 | NLIDSYFV | L2 | SFLNGSCG | L72; L71 | MLAKALRKV | L3; L2; L4; L24; |
| | | | | | | L64 |
| 2498 | NLKYAISA | L23 | SFLNRVCGV | L71; L72 | MLNPNYEDL | L5; L4; L3; L74; L2 |
| 2499 | NLKYAISAK | L6 | SFLPGVYS | L71; L72 | MMSAPPAQY | L14; L12; L26; |
| | | | | | | L31; L16 |
| 2500 | NLLLDKRTT | L23 | SFLPGVYSVI | L8; L9 | MPASWVMRI | L47; L49; L32; |
| | | | | | | L50; L20 |
| 2501 | NLPFKLTC | L57 | SFPQSAPH | L71; L72 | MPILTLTRAL | L32; L22; L31; |
| | | | | | | L50; L39 |
| 2502 | NLPGCDGGSL | L57 | SFSTFEEAAL | L72; L71 | MPLKAPKEI | L47; L50; L39; |
| | | | | | | L32; L49 |
| 2503 | NLPLQLGF | L57 | SFSTFKCY | L72; L71 | MPLSAPTLV | L47; L50; L51; |
| | | | | | | L22; L31 |
| 2504 | NLPTMCDI | L57 | SFVIRGDEV | L72; L71 | MSKFPLKL | L70; L73; L55; |
| | | | | | | L74; L69 |
| 2505 | NLPTMCDIRQL | L57 | SFYVYANGG | L72; L71 | MVELVAEL | L68; L62; L63; |
| | | | | | | L67; L59 |
| 2506 | NLREFVFK | L13 | SGDATTAY | L1; L18 | MVPHISRQR | L19; L17; L15; |
| | | | | | | L11; L21 |
| 2507 | NLREFVFKNI | L3 | SGDGTTSPI | L68; L63 | MVVIPDYNTY | L10; L11; L26; |
| | | | | | | L12; L14 |
| 2508 | NLREMLAH | L43 | SGLKTILR | L15; L17 | MWALIISV | L50; L25; L47; |
| | | | | | | L28; L33 |
| 2509 | NLSKSLTEN | L3 | SGLKTILRK | L7; L6 | MYIFFASFY | L72; L12; L71; |
| | | | | | | L14; L66 |
| 2510 | NLVAMKYNY | L12 | SGTNGTKRF | L49; L27 | NCVADYSVL | L35; L25; L28; |
| | | | | | | L10; L67 |
| 2511 | NMFITREE | L48 | SGVPVVDSYY | L14; L12 | NCYDYCIPY | L43; L69; L6; L72; |
| | | | | | | L71 |
| 2512 | NMFTPLIQP | L46 | SGYLKLTD | L48; L65 | NEEIAIIL | L29; L25; L37; |
| | | | | | | L36; L45 |
| 2513 | NMLRIMASL | L8 | SHAAVDALC | L34; L28 | NEKCSAYTV | L37; L45; L42; |
| | | | | | | L38; L29 |
| 2514 | NNATNKATY | L12 | SHFAIGLALYY | L12; L41 | NELSPVALR | L17; L40; L41; |
| | | | | | | L19; L15 |
| 2515 | NNATNVVIK | L19 | SHNIALIW | L35; L28 | NESGLKTI | L45; L48; L29; |
| | | | | | | L37; L25 |
| 2516 | NNLDSKVGGNY | L14 | SHQSDIEV | L28; L35 | NESLIDLQEL | L40; L41; L36; |
| | | | | | | L29; L37 |
| 2517 | NPDILRVYA | L50 | SHSQLGGL | L28; L35 | NFNQHEVL | L71; L25; L72; |
| | | | | | | L62; L23 |
| 2518 | NPIQLSSY | L31 | SHSQLGGLH | L28; L34 | NFNVLFSTVF | L62; L9; L72; L71; |
| | | | | | | L8 |
| 2519 | NPKAIKCVPQA | L51 | SHTVMPLSA | L28; L35 | NFTIKGSFL | L72; L71; L8; L62; |
| | | | | | | L9 |
| 2520 | NPKTPKYKFV | L39 | SIAIPTNF | L74; L26 | NGGDAALAL | L67; L61; L35; |
| | | | | | | L66; L57 |
| 2521 | NPLLYDANY | L31 | SIDAFKLNIK | L7; L6 | NIDGYFKIY | L18; L1; L14; L58; |
| | | | | | | L29 |
| 2522 | NPLLYDANYF | L31 | SIVAGGIVAI | L10; L20 | NIFGTVYEK | L7; L19; L6; L17; |
| | | | | | | L13 |
| 2523 | NPTDQSSY | L31 | SKEGFFTY | L27; L66 | NIIPLTTAA | L28; L3; L11; L50; |
| | | | | | | L46 |
| 2524 | NPVLPFNDGVY | L31 | SKHTPINLV | L28; L64 | NPANNAAIV | L47; L22; L50; |
| | | | | | | L39; L51 |
| 2525 | NPVLPFNDGVYF | L31 | SKIITLKKRW | L41; L40 | NPANNAAIVL | L56; L39; L31; |
| | | | | | | L22; L32 |
| 2526 | NPVMEPIY | L31 | SKSLTENKY | L27; L14 | NPAWRKAV | L39; L25; L23; |
| | | | | | | L22; L51 |
| 2527 | NPVMEPIYD | L49 | SKTPEEHFI | L44; L34 | NPIQLSSYSL | L56; L22; L32; |
| | | | | | | L39; L31 |
| 2528 | NQFNSAIGKI | L24 | SKTTVASL | L44; L66 | NPKTPKYKF | L39; L49; L23; |
| | | | | | | L31; L22 |
| 2529 | NQNAQALNTL | L34 | SKVGGNYNY | L27; L14 | NQDLNGNWY | L1; L26; L27; L18; |
| | | | | | | L68 |
| 2530 | NQRNAPRI | L48 | SKVQIGEY | L27; L66 | NRALTGIAV | L35; L25; L30; |
| | | | | | | L28; L64 |
| 2531 | NRARTVAG | L35 | SLAATVRLQ | L3; L64 | NSSPDDQIGY | L1; L18; L52; L54; |
| | | | | | | L14 |
| 2532 | NRATLQAIA | L35 | SLAEYHNES | L2; L3 | NSSRVPDLL | L74; L67; L53; |
| | | | | | | L73; L20 |
| 2533 | NRATRVEC | L25 | SLAGSYKDWSY | L14; L18 | NTQEVFAQV | L20; L13; L73; L5; |
| | | | | | | L11 |
| 2534 | NRATRVECT | L25 | SLAIDAYPL | L2; L4 | NVVRIKIVQM | L19; L11; L20; |
| | | | | | | L32; L10 |
| 2535 | NRFLYIIK | L25 | SLAIDAYPLTK | L6; L7 | NYMPYFFTLL | L9; L8; L71; L66; |
| | | | | | | L27 |
| 2536 | NRFLYIIKLIF | L30 | SLDTYPSLETI | L68; L5 | NYQHEETIY | L72; L66; L71; |
| | | | | | | L14; L12 |
| 2537 | NRFNVAITRA | L30 | SLFDMSKFPL | L2; L4 | PEVKILNNL | L37; L36; L29; |
| | | | | | | L41; L45 |
| 2538 | NRFTTTLND | L65 | SLIDFYLCFLAF | L12; L10 | PFNDGVYF | L62; L72; L9; L71; |
| | | | | | | L8 |
| 2539 | NRNRFLYI | L35 | SLKEGQIND | L13; L43 | PLVPFWITI | L3; L2; L5; L4; L69 |
| 2540 | NRNRFLYIIK | L30 | SLKEGQINDM | L11; L3 | PQLEQPYVF | L8; L9; L16; L27; |
| | | | | | | L26 |
| 2541 | NRNRFLYIIKL | L30 | SLKELLQN | L3; L43 | PVVDSYYSL | L10; L67; L20; |
| | | | | | | L60; L61 |
| 2542 | NRNYVFTG | L65 | SLKITEEV | L23; L3 | QAIASEFSSL | L61; L67; L60; |
| | | | | | | L10; L32 |
| 2543 | NRPQIGVVREF | L66 | SLLSKGRLIIR | L21; L15 | QAISMWAL | L67; L61; L59; |
| | | | | | | L70; L60 |
| 2544 | NRQFHQKLLK | L30 | SLLVLVQST | L4; L2 | QASLPFGWL | L67; L61; L60; |
| | | | | | | L59; L74 |
| 2545 | NRVCGVSAAR | L30 | SLPINVIV | L57; L5 | QELGKYEQY | L29; L41; L40; |
| | | | | | | L42; L36 |
| 2546 | NRVCTNYMPY | L66 | SLPSYAAFA | L5; L57 | QEYADVFHLY | L41; L40; L14; |
| | | | | | | L29; L42 |
| 2547 | NRVVISSD | L25 | SLRCGACIR | L21; L15 | QLFFSYFAV | L4; L24; L3; L2; L5 |
| 2548 | NRVVISSDVL | L35 | SLREVRTIKVF | L10; L43 | QLLFVVEVV | L4; L2; L24; L3; L5 |
| 2549 | NRYFRLTLGVY | L30 | SLRPDTRYVL | L16; L13 | QLMCQPILL | L74; L4; L2; L5; L3 |
| 2550 | NRYLALYN | L25 | SLSEQLRK | L6; L7 | QLPAPRTL | L57; L64; L55; L5; |
| | | | | | | L66 |
| 2551 | NRYLALYNKY | L30 | SLSHRFYR | L21; L15 | QLYLGGMSYY | L18; L1; L6; L14; |
| | | | | | | L26 |
| 2552 | NSEVGPEHSL | L68 | SLTIKKPNEL | L3; L4 | QMEIDFLEL | L1; L67; L34; L68; |
| | | | | | | L18 |
| 2553 | NSHEGKTFYVL | L67 | SLVKPSFY | L26; L14 | QPYPNASF | L22; L39; L47; |
| | | | | | | L31; L56 |
| 2554 | NSIAIPTN | L48 | SLWVYKQF | L43; L26 | QQFGPTYL | L48; L28; L74; |
| | | | | | | L69; L25 |
| 2555 | NSIIKTIQPR | L19 | SLYVKPGGT | L43; L3 | QSLENVAF | L54; L52; L55; |
| | | | | | | L53; L33 |
| 2556 | NSIVCRFDTR | L19 | SNYQHEETI | L34; L28 | QTMLFTMLR | L21; L15; L17; L7; |
| | | | | | | L19 |
| 2557 | NSNNLDSKV | L73 | SPDAVTAYN | L49; L22 | QVDVVNFNL | L68; L5; L63; L1; |
| | | | | | | L26 |
| 2558 | NSPFHPLAD | L57 | SPDVDLGDI | L49; L22 | QVVNVVTTK | L19; L7; L6; L13; |
| | | | | | | L20 |
| 2559 | NSQGLLPPK | L7 | SPFHPLADNKF | L31; L49 | RANNTKGSL | L56; L44; L57; |
| | | | | | | L58; L61 |
| 2560 | NSVLLFLAFV | L20 | SPFVMMSAP | L51; L22 | RARSVSPKLF | L52; L53; L55; |
| | | | | | | L58; L27 |
| 2561 | NSVRVLQK | L19 | SPIFLIVAAI | L22; L50 | RCLAVHECF | L54; L8; L33; L53; |
| | | | | | | L52 |
| 2562 | NSVTSSIV | L70 | SPIFLIVAAIVF | L31; L41 | RDGWEIVKF | L33; L65; L53; |
| | | | | | | L37; L54 |
| 2563 | NSVTSSIVI | L70 | SPKLFIRQ | L39; L51 | REAVGTNL | L33; L37; L36; |
| | | | | | | L45; L56 |
| 2564 | NSWLMWLII | L67 | SPKLFIRQE | L39; L51 | REEAIRHVR | L38; L42; L36; |
| | | | | | | L37; L40 |
| 2565 | NTCTERLKLF | L10 | SPLYAFASE | L51; L50 | REETGLLM | L45; L33; L36; |
| | | | | | | L37; L18 |
| 2566 | NTDHSSSS | L1 | SPLYAFASEA | L51; L50 | REFLTRNPAW | L40; L41; L36; |
| | | | | | | L38; L37 |
| 2567 | NTKGSLPINV | L20 | SPNFSKLI | L39; L47 | REFVFKNI | L45; L48; L33; |
| | | | | | | L37; L29 |
| 2568 | NTLNDLNETL | L53 | SPNFSKLINI | L56; L39 | RELKVTFF | L33; L29; L37; |
| | | | | | | L27; L36 |
| 2569 | NTSNQVAV | L20 | SPNLAWPLIVTA | L50; L51 | RENNRVVIS | L33; L38; L42; |
| | | | | | | L37; L46 |
| 2570 | NTSRYWEPEF | L53 | SPRRARSVA | L51; L22 | REVGFVVPG | L38; L46; L37; |
| | | | | | | L36; L42 |
| 2571 | NTSRYWEPEFY | L1 | SPYNSQNAVA | L51; L50 | REVGFVVPGL | L36; L37; L41; |
| | | | | | | L40; L45 |
| 2572 | NTTKGGRF | L54 | SQASSRSSSR | L21; L15 | RFNGIGVTQ | L8; L15; L71; L56; |
| | | | | | | L72 |
| 2573 | NTTKGGRFV | L20 | SQLDEEQPM | L24; L44 | RFNVAITR | L15; L71; L21; |
| | | | | | | L17; L72 |
| 2574 | NTTKGGRFVL | L16 | SQRGGSYTN | L26; L27 | RFRRAFGEY | L14; L13; L27; |
| | | | | | | L72; L12 |
| 2575 | NTVCTVCGM | L11 | SQRVAGDSGF | L26; L27 | RFTTTLNDF | L8; L9; L72; L71; |
| | | | | | | L62 |
| 2576 | NTVKSVGK | L19 | SREETGLL | L66; L35 | RGDEVRQI | L55; L48; L63; |
| | | | | | | L33; L45 |
| 2577 | NTYLEGSVRV | L20 | SRGTSPARM | L66; L30 | RHHANEYRL | L44; L34; L35; |
| | | | | | | L28; L56 |
| 2578 | NVAFELWAKR | L19 | SRILGAGCF | L30; L34 | RIDKVLNEK | L6; L7; L18; L13; |
| | | | | | | L5 |
| 2579 | NVAFQTVK | L19 | SRLDKVEAE | L30; L35 | RIFTIGTVT | L44; L13; L6; L21; |
| | | | | | | L16 |
| 2580 | NVAFQTVKP | L19 | SRYRIGNY | L65; L66 | RKAVFISPY | L27; L26; L43; |
| | | | | | | L18; L6 |
| 2581 | NVAITRAKV | L20 | SRYRIGNYK | L30; L6 | RLKLFDRYF | L26; L55; L27; |
| | | | | | | L14; L52 |
| 2582 | NVAKYTQLCQY | L11 | SSFLEMKSEK | L7; L13 | RQFHQKLLK | L6; L24; L13; L7; |
| | | | | | | L30 |
| 2583 | NVATLQAENV | L20 | SSKCVCSV | L70; L48 | RQIAPGQTGK | L6; L7; L30; L13; |
| | | | | | | L21 |
| 2584 | NVDRYPANSI | L68 | SSKLWAQCV | L13; L70 | RSFIEDLL | L55; L53; L54; |
| | | | | | | L65; L73 |
| 2585 | NVGDYFVLT | L20 | SSRVPDLLV | L73; L13 | RSLPGVFCG | L54; L52; L55; |
| | | | | | | L16; L53 |
| 2586 | NVIPTITQMNL | L20 | SSSSDNIALL | L53; L20 | RSVSPKLF | L55; L54; L52; |
| | | | | | | L53; L73 |
| 2587 | NVIVFDGKSK | L19 | SSTASALGK | L7; L19 | RTAPHGHVMV | L73; L18; L20; |
| | | | | | | L53; L13 |
| 2588 | NVKTTEVV | L23 | SSYIVDSV | L48; L47 | RTFKVSIW | L54; L16; L52; |
| | | | | | | L53; L55 |
| 2589 | NVLAWLYAA | L50 | SSYIVDSVTV | L48; L73 | RTFKVSIWN | L53; L13; L54; |
| | | | | | | L55; L7 |
| 2590 | NVLEGSVA | L25 | SSYIVDSVTVK | L7; L6 | RTIKGTHHWL | L53; L16; L55; |
| | | | | | | L54; L73 |
| 2591 | NVLSTFISAAR | L17 | STDGNKIAD | L68; L1 | RTLSYYKL | L55; L54; L53; |
| | | | | | | L73; L16 |
| 2592 | NVLTLVYKVYY | L12 | STDVVYRAFDIY | L1; L18 | RTRSMWSF | L55; L53; L16; |
| | | | | | | L54; L52 |
| 2593 | NVPLHGTILTR | L19 | STECSNLL | L1; L63 | RVAGDSGFAAY | L14; L18; L16; L6; |
| | | | | | | L26 |
| 2594 | NVTDFNAI | L20 | STECSNLLLQY | L1; L18 | RVCGVSAAR | L21; L15; L7; L19; |
| | | | | | | L6 |
| 2595 | NVTDFNAIA | L20 | STGVNLVAV | L73; L20 | RVCTNYMPY | L18; L6; L14; L13; |
| | | | | | | L16 |
| 2596 | NVTWFHAIHV | L20 | STIGVCSM | L70; L58 | RVFSAVGNI | L16; L24; L3; L6; |
| | | | | | | L10 |
| 2597 | NVVNKGHFD | L11 | STKPVETSN | L43; L53 | RVYSSANNC | L16; L6; L21; L15; |
| | | | | | | L14 |
| 2598 | NVVRIKIVQ | L20 | STLEQYVFC | L7; L13 | RYPANSIVC | L72; L71; L8; L9; |
| | | | | | | L66 |
| 2599 | NVYIKNADI | L25 | STLQGPPGTGK | L6; L7 | SAFAMMFV | L47; L70; L69; |
| | | | | | | L58; L73 |
| 2600 | NVYLKHGGGV | L11 | STMTNRQFHQK | L7; L6 | SALVYDNKL | L32; L74; L61; |
| | | | | | | L67; L60 |
| 2601 | NWITGGIAIAM | L71 | STNLVKNK | L13; L7 | SAPLIELCV | L64; L57; L69; |
| | | | | | | L47; L70 |
| 2602 | NWNCVNCDTF | L8 | STPCNGVEGF | L11; L10 | SAQCFKMFY | L58; L14; L59; |
| | | | | | | L69; L12 |
| 2603 | NYDLSVVN | L62 | STQDLFLPFF | L52; L12 | SEAGVCVST | L42; L38; L36; |
| | | | | | | L37; L41 |
| 2604 | NYDLSVVNA | L62 | STSHKLVL | L73; L70 | SEDKRAKVT | L45; L38; L42; |
| | | | | | | L37; L40 |
| 2605 | NYDLSVVNAR | L17 | STSHKLVLSV | L20; L73 | SEDMLNPNY | L41; L29; L40; |
| | | | | | | L36; L1 |
| 2606 | NYDLSVVNARL | L62 | STYASQGL | L60; L25 | SEEVVENPTI | L36; L41; L38; |
| | | | | | | L45; L42 |
| 2607 | NYEDLLIR | L17 | SVAYSNNSI | L20; L10 | SEFDRDAA | L46; L42; L37; |
| | | | | | | L29; L38 |
| 2608 | NYITTYPG | L71 | SVDCTMYI | L68; L63 | SEGLNDNLL | L36; L40; L41; |
| | | | | | | L37; L45 |
| 2609 | NYLYRLFR | L17 | SVELKHFF | L63; L68 | SEHDYQIGGY | L40; L41; L14; |
| | | | | | | L11; L29 |
| 2610 | NYMLTYNKV | L62 | SVELKHFFF | L12; L63 | SEISMDNSP | L42; L38; L46; |
| | | | | | | L37; L36 |
| 2611 | NYQCGHYKH | L8 | SVLHSTQDL | L60; L56 | SEQLRKQI | L45; L48; L33; |
| | | | | | | L29; L37 |
| 2612 | NYQVNGYPNM | L71 | SVLHSTQDLF | L12; L52 | SEQLRKQIR | L42; L38; L40; |
| | | | | | | L33; L29 |
| 2613 | NYVFTGYR | L17 | SVLQQLRV | L48; L24 | SEYDYVIFTQ | L42; L37; L40; |
| | | | | | | L41; L38 |
| 2614 | NYVFTGYRV | L8 | SVNITFELD | L65; L49 | SFNPETNILL | L62; L8; L9; L71; |
| | | | | | | L72 |
| 2615 | NYYKKVDGV | L71 | SVNPYVCNA | L65; L3 | SGFAAYSRY | L14; L27; L12; |
| | | | | | | L10; L26 |
| 2616 | PANSIVCR | L17 | SVRVLQKAA | L51; L13 | SHKLVLSV | L48; L28; L23; |
| | | | | | | L35; L51 |
| 2617 | PAQLPAPRTL | L60 | SVYAWNRK | L7; L6 | SHLLLVAA | L50; L35; L25; |
| | | | | | | L28; L51 |
| 2618 | PARARVECF | L58 | SVYPVASP | L51; L43 | SHVDTDLTK | L34; L7; L28; L35; |
| | | | | | | L19 |
| 2619 | PAVAKHDFF | L58 | SVYPVASPN | L6; L43 | SICSTMTNR | L21; L15; L19; |
| | | | | | | L17; L7 |
| 2620 | PCIKVATV | L48 | SVYYSQLMC | L6; L7 | SIDAFKLNI | L5; L68; L63; L34; |
| | | | | | | L24 |
| 2621 | PCSDKAYKI | L8 | SWEVGKPRP | L42; L38 | SIKNFKSV | L23; L39; L13; |
| | | | | | | L43; L48 |
| 2622 | PDGVKHVY | L65 | SWFTALTQH | L7; L6 | SIVAGGIVA | L51; L46; L38; |
| | | | | | | L28; L42 |
| 2623 | PDILRVYAN | L33 | SWFTALTQHGK | L7; L6 | SLFFFLYEN | L4; L3; L2; L24; |
| | | | | | | L12 |
| 2624 | PEEEQEEDWL | L36 | SWLMWLIINL | L4; L8 | SLIDFYLCFL | L4; L2; L3; L5; L20 |
| 2625 | PEEHVQIHTI | L45 | SWMESEFRV | L4; L2 | SLREVRTI | L48; L3; L55; L23; |
| | | | | | | L24 |
| 2626 | PEFYEAMY | L29 | SWNLREML | L62; L71 | SLTENKYSQL | L3; L10; L2; L4; L5 |
| 2627 | PEHSLAEY | L29 | SYAAFATAQ | L71; L72 | SLVPGFNEK | L7; L6; L13; L19; |
| | | | | | | L21 |
| 2628 | PEYFNSVCRL | L37 | SYEDQDALFAY | L12; L14 | SPDDQIGY | L49; L1; L18; L31; |
| | | | | | | L65 |
| 2629 | PFAMQMAYR | L17 | SYFTEQPI | L71; L72 | SPFELEDFI | L47; L49; L34; |
| | | | | | | L31; L32 |
| 2630 | PFAMQMAYRF | L9 | SYFTSDYYQLY | L14; L12 | SPNLAWPLIV | L22; L50; L51; |
| | | | | | | L47; L39 |
| 2631 | PFDVVRQC | L62 | SYGIATVR | L15; L17 | SQLGGLHL | L48; L25; L35; |
| | | | | | | L27; L24 |
| 2632 | PFELEDFI | L62 | SYIVDSVT | L71; L72 | SQYSLRLI | L48; L24; L64; |
| | | | | | | L25; L47 |
| 2633 | PFFSNVTW | L71 | SYIVDSVTVK | L13; L7 | SREETGLLM | L34; L35; L30; |
| | | | | | | L66; L65 |
| 2634 | PFLGVYYHK | L8 | SYLFQHAN | L71; L72 | SRLSFKEL | L25; L66; L35; |
| | | | | | | L30; L64 |
| 2635 | PFLNKVVST | L71 | SYQTQTNSP | L71; L72 | SSANNCTF | L54; L53; L43; |
| | | | | | | L58; L70 |
| 2636 | PFLYLYALVY | L12 | SYSLFDMSKF | L9; L8 | SSANNCTFEY | L18; L1; L43; L58; |
| | | | | | | L14 |
| 2637 | PFMIDVQQWGF | L8 | SYTNDKACPL | L72; L71 | SSKTPEEHF | L43; L58; L52; |
| | | | | | | L53; L26 |
| 2638 | PFNKWGKARL | L8 | SYTTTIKPV | L72; L71 | SSQCVNLTTR | L19; L15; L21; |
| | | | | | | L17; L7 |
| 2639 | PFNKWGKARLY | L14 | SYYKLGASQ | L72; L71 | SSRSSSRSR | L15; L21; L17; |
| | | | | | | L13; L19 |
| 2640 | PFWITIAYI | L62 | TAESHVDTD | L49; L32 | STCMMCYKR | L17; L7; L15; L21; |
| | | | | | | L19 |
| 2641 | PGQTGKIADY | L14 | TAESHVDTDL | L32; L63 | STDTCFANK | L13; L7; L19; L1; |
| | | | | | | L6 |
| 2642 | PGSGVPVVD | L65 | TAHSCNVNRF | L49; L58 | STDTGVEH | L63; L68; L1; L62; |
| | | | | | | L18 |
| 2643 | PHNSHEGKTF | L34 | TAPHGHVMVEL | L57; L67 | STDTGVEHVTF | L53; L68; L63; |
| | | | | | | L54; L52 |
| 2644 | PHVGEIPVA | L28 | TAVVIPTKK | L19; L7 | STEKSNIIR | L17; L19; L21; |
| | | | | | | L15; L7 |
| 2645 | PIDKCSRI | L63 | TCYFGLFCL | L34; L35 | STFISDEVAR | L7; L19; L15; L17; |
| | | | | | | L21 |
| 2646 | PIDLVPNQPY | L1 | TDDNALAY | L1; L18 | STFNVPMEKL | L20; L53; L11; |
| | | | | | | L10; L16 |
| 2647 | PIFLIVAA | L50 | TDFSRVSAK | L33; L6 | STGSNVFQTR | L19; L21; L15; L7; |
| | | | | | | L17 |
| 2648 | PIHFYSKWY | L43 | TDGTLMIER | L19; L17 | STQLGIEFL | L20; L53; L60; |
| | | | | | | L73; L68 |
| 2649 | PIIGDELKI | L24 | TDLTKGPHEF | L33; L8 | STQWSLFFFLY | L14; L12; L1; L18; |
| | | | | | | L7 |
| 2650 | PILLLDQAL | L67 | TDVTQLYL | L65; L33 | SVAALTNNV | L20; L3; L64; L58; |
| | | | | | | L4 |
| 2651 | PILTLTRAL | L60 | TDVVYRAF | L33; L29 | SVAIKITEH | L11; L43; L6; L7; |
| | | | | | | L10 |
| 2652 | PIQYIDIGNY | L14 | TDYKHWPQI | L33; L47 | SVIDLLLDDF | L11; L10; L12; |
| | | | | | | L52; L14 |
| 2653 | PISAMVRMY | L14 | TEAFEKMVS | L46; L38 | SVLLFLAFV | L4; L20; L73; L2; |
| | | | | | | L5 |
| 2654 | PKGPKVKYL | L64 | TECSNLLLQY | L41; L40 | SVLNDILSRL | L4; L10; L11; L6; |
| | | | | | | L14 |
| 2655 | PKLGSLVVR | L17 | TEERLKLFD | L45; L42 | SVNITFEL | L23; L68; L59; |
| | | | | | | L56; L65 |
| 2656 | PKSDGTGTIY | L27 | TEGALNTP | L38; L46 | SYLFQHANL | L72; L9; L8; L71; |
| | | | | | | L62 |
| 2657 | PLIVTALR | L21 | TEGLCVDIP | L38; L42 | TAAKLMVV | L70; L47; L69; |
| | | | | | | L23; L58 |
| 2658 | PLKLRGTAV | L23 | TEIDPKLD | L45; L29 | TCANDPVGF | L34; L9; L31; L26; |
| | | | | | | L28 |
| 2659 | PLKSATCITR | L21 | TEIDPKLDNYY | L41; L40 | TCFSVAAL | L35; L28; L71; |
| | | | | | | L72; L56 |
| 2660 | PLLESELV | L2 | TERSEKSYEL | L36; L37 | TCTERLKLF | L40; L9; L8; L10; |
| | | | | | | L41 |
| 2661 | PLLYDANYF | L9 | TESIVRFP | L33; L38 | TEDDYQGKPL | L36; L41; L45; |
| | | | | | | L40; L37 |
| 2662 | PLSETKCTL | L2 | TESIVRFPNI | L45; L41 | TEERLKLF | L29; L33; L37; |
| | | | | | | L41; L40 |
| 2663 | PLYAFASEA | L3 | TESNKKFLPF | L41; L40 | TEHSWNADL | L37; L36; L41; |
| | | | | | | L40; L45 |
| 2664 | PMEKLKTL | L23 | TETDLTKGPHEF | L41; L40 | TEIDPKLDNY | L41; L40; L29; |
| | | | | | | L14; L36 |
| 2665 | PNFKDQVIL | L44 | TEVNEFACVVA | L46; L38 | TEILPVSMT | L36; L38; L41; |
| | | | | | | L45; L46 |
| 2666 | PNLAWPLIV | L73 | TEVPANST | L29; L45 | TEKSNIIRGW | L40; L41; L10; |
| | | | | | | L45; L37 |
| 2667 | PNYEDLLIR | L17 | TEVPANSTVLSF | L41; L40 | TENKYSQLD | L40; L65; L38; |
| | | | | | | L45; L46 |
| 2668 | PPGDQFKHL | L39 | TEVPVAIHADQL | L36; L41 | TEQPIDLV | L33; L45; L29; |
| | | | | | | L48; L38 |
| 2669 | PPKNSIDA | L51 | TEVVGDII | L45; L36 | TEQPIDLVP | L38; L42; L46; |
| | | | | | | L40; L41 |
| 2670 | PPLNRNYV | L47 | TFCAGSTFI | L8; L62 | TETAHSCNV | L45; L38; L46; |
| | | | | | | L33; L37 |
| 2671 | PPLNRNYVF | L8 | TFLLNKEM | L72; L71 | TEVNEFACV | L45; L37; L46; |
| | | | | | | L38; L36 |
| 2672 | PQADVEWKFY | L14 | TFPPTEPK | L71; L72 | TFDNLKTL | L62; L68; L71; |
| | | | | | | L72; L63 |
| 2673 | PQIITTDNTF | L26 | TFPPTEPKK | L8; L9 | TFHLDGEVI | L8; L72; L34; L9; |
| | | | | | | L71 |
| 2674 | PQLEQPYV | L48 | TFQSAVKR | L17; L15 | TFKVSIWNL | L72; L8; L62; L9; |
| | | | | | | L17 |
| 2675 | PQNQRNAPR | L21 | TFSVLACY | L72; L71 | TFNGECPNF | L9; L8; L62; L71; |
| | | | | | | L72 |
| 2676 | PQTLEILDI | L24 | TFTCASEY | L72; L71 | TFNVPMEKL | L62; L8; L72; L9; |
| | | | | | | L71 |
| 2677 | PQTSITSAV | L24 | TFTRLQSL | L71; L72 | TFYLTNDVSF | L62; L9; L72; L71; |
| | | | | | | L8 |
| 2678 | PQTSITSAVL | L44 | TFTRSTNSR | L17; L19 | TGPEAGLPY | L57; L72; L66; |
| | | | | | | L12; L71 |
| 2679 | PQVNGLTSI | L24 | TFVTHSKGLY | L14; L12 | THHWLLLTI | L34; L35; L28; |
| | | | | | | L24; L47 |
| 2680 | PREGVFVS | L65 | TGTIYTEL | L23; L25 | TIAEILLI | L24; L48; L47; L3; |
| | | | | | | L73 |
| 2681 | PRPPLNRNYV | L64 | TGTLIVNSV | L48; L64 | TICAPLTVF | L26; L10; L58; |
| | | | | | | L59; L39 |
| 2682 | PRVEKKKL | L25 | TGTSTDVVY | L14; L27 | TKFLTENLL | L44; L34; L64; |
| | | | | | | L27; L28 |
| 2683 | PSDSTGSNQ | L1 | TGTSTDVVYR | L19; L17 | TLAILTALR | L21; L19; L17; |
| | | | | | | L15; L6 |
| 2684 | PSFKKGAKL | L53 | TGVNLVAV | L48; L23 | TLDSKTQSLL | L63; L1; L5; L2; |
| | | | | | | L68 |
| 2685 | PSFLGRYM | L55 | THGLAAVNSV | L28; L35 | TLKNTVCTV | L3; L2; L4; L24; |
| | | | | | | L23 |
| 2686 | PSFYVYSRVK | L13 | THSDKFTD | L35; L65 | TLLLQLCTF | L26; L9; L16; L27; |
| | | | | | | L12 |
| 2687 | PSLATVAYF | L52 | THSKGLYRK | L34; L35 | TLLSLREV | L4; L23; L2; L3; |
| | | | | | | L48 |
| 2688 | PSLETIQI | L48 | THVQLSLPV | L28; L35 | TLMNVLTLVY | L12; L6; L14; L18; |
| | | | | | | L26 |
| 2689 | PSVEQRKQ | L55 | THWFVTQR | L17; L19 | TLNDLNETLV | L2; L4; L3; L5; L24 |
| 2690 | PSYAAFATA | L46 | TICAPLTV | L48; L73 | TLQQIELKF | L12; L26; L16; L9; |
| | | | | | | L8 |
| 2691 | PTDQSSYI | L1 | TIDGSSGV | L63; L68 | TLRVEAFEY | L26; L43; L12; |
| | | | | | | L14; L1 |
| 2692 | PTITQMNLK | L7 | TIDYTEISFM | L63; L62 | TNYDLSVV | L48; L25; L47; |
| | | | | | | L73; L69 |
| 2693 | PTNGVGYQPY | L14 | TIEVNSFSGY | L1; L18 | TPSDFVRAT | L32; L51; L31; |
| | | | | | | L39; L49 |
| 2694 | PVLKGVKLHY | L12 | TIKVFTTV | L23; L48 | TPVCINGLML | L32; L56; L22; |
| | | | | | | L31; L39 |
| 2695 | PVLPFNDGVY | L14 | TINCQEPKL | L4; L32 | TQDLFLPFF | L62; L34; L68; |
| | | | | | | L63; L58 |
| 2696 | PVLPFNDGVYF | L52 | TIPIQASL | L57; L23 | TQFNYYKKV | L24; L48; L64; L3; |
| | | | | | | L69 |
| 2697 | PVPEVKILN | L5 | TIQPRVEKK | L7; L6 | TREAVGTNL | L35; L34; L30; |
| | | | | | | L28; L66 |
| 2698 | PVPEVKILNNL | L5 | TIQTIVEV | L23; L48 | TSITSAVL | L61; L55; L65; |
| | | | | | | L54; L60 |
| 2699 | PVSIINNTV | L20 | TISVTTEIL | L34; L28 | TSNQVAVL | L65; L73; L48; |
| | | | | | | L55; L70 |
| 2700 | PYFFTLLLQ | L9 | TITSGWTF | L54; L55 | TSRYWEPEF | L43; L65; L52; |
| | | | | | | L53; L58 |
| 2701 | PYGANKDGI | L9 | TITVNVLAWLY | L12; L14 | TTFDSEYCR | L19; L17; L15; |
| | | | | | | L21; L7 |
| 2702 | PYIKWDLL | L9 | TIVNGVRR | L19; L17 | TTNIVTRCL | L55; L65; L73; |
| | | | | | | L64; L60 |
| 2703 | PYNSVTSSIVI | L9 | TIVNGVRRSF | L10; L16 | TTTLNGLWL | L60; L61; L73; |
| | | | | | | L55; L1 |
| 2704 | PYPDPSRILG | L9 | TKVDGVDVELF | L27; L34 | TTVDNINLH | L19; L11; L20; L7; |
| | | | | | | L10 |
| 2705 | PYPNASFDN | L9 | TLATCELY | L18; L1 | TVATSRTL | L55; L65; L39; |
| | | | | | | L28; L23 |
| 2706 | PYVFIKRSD | L65 | TLAVPYNMRV | L2; L4 | TVKGLDYKAF | L10; L39; L26; |
| | | | | | | L43; L11 |
| 2707 | PYVVDDPCPIHF | L9 | TLDNQDLNGNW | L1; L18 | TVLCLTPVY | L12; L14; L31; |
| | | | Y | | | L52; L16 |
| 2708 | QAENVTGL | L63 | TLGVYDYLV | L2; L4 | TVSWNLREM | L32; L64; L58; |
| | | | | | | L39; L59 |
| 2709 | QAGNATEV | L70 | TLIGEAVKT | L24; L2 | TVYSHLLL | L25; L74; L67; |
| | | | | | | L73; L23 |
| 2710 | QAGNVQLRVI | L55 | TLKEILVT | L23; L43 | TYPGQGLNGY | L14; L72; L12; |
| | | | | | | L71; L8 |
| 2711 | QAISMWALI | L47 | TLKGGAPTK | L13; L6 | TYPSLETI | L9; L8; L71; L72; |
| | | | | | | L62 |
| 2712 | QAISMWALII | L69 | TLKGGAPTKV | L3; L2 | VAIDYKHYT | L69; L67; L70; |
| | | | | | | L58; L43 |
| 2713 | QALPQRQK | L55 | TLKGVEAV | L3; L23 | VAPGTAVLR | L19; L7; L15; L17; |
| | | | | | | L21 |
| 2714 | QALPQRQKK | L13 | TLKKRWQL | L23; L39 | VAYFNMVY | L59; L70; L69; |
| | | | | | | L43; L58 |
| 2715 | QAPTHLSVD | L57 | TLKKRWQLA | L23; L3 | VCINGLML | L61; L71; L60; |
| | | | | | | L72; L74 |
| 2716 | QCIMLVYCF | L34 | TLKNLSDRV | L3; L2 | VECFDKFKV | L37; L45; L38; |
| | | | | | | L42; L36 |
| 2717 | QDDKKIKAC | L33 | TLLFLMSFTV | L2; L4 | VEEAKKVKP | L38; L42; L46; |
| | | | | | | L45; L37 |
| 2718 | QDKNTQEV | L33 | TLLPAADL | L23; L74 | VEEAKTVL | L37; L36; L45; |
| | | | | | | L23; L25 |
| 2719 | QDLFLPFF | L33 | TLMIERFVSL | L2; L4 | VEELKKLL | L45; L33; L23; |
| | | | | | | L37; L36 |
| 2720 | QDLKWARF | L33 | TLMNVLTLVYK | L6; L7 | VELFENKTTL | L36; L37; L45; |
| | | | | | | L41; L40 |
| 2721 | QDLSVVSKV | L33 | TLNDFNLV | L4; L2 | VESCGNFKV | L45; L37; L38; |
| | | | | | | L42; L36 |
| 2722 | QEGVVDYGARF | L40 | TLNISDEF | L26; L62 | VETKDVVEC | L37; L45; L36; |
| | Y | | | | | L29; L38 |
| 2723 | QEHYVRIT | L29 | TLPTEVLTE | L57; L5 | VFHLYLQYI | L62; L8; L9; L71; |
| | | | | | | L72 |
| 2724 | QEILGTVS | L29 | TLPVNVAFEL | L57; L5 | VFKNIDGYF | L9; L8; L12; L62; |
| | | | | | | L72 |
| 2725 | QEKDEDDNL | L36 | TLQGPPGTGK | L6; L7 | VFVSNGTHW | L9; L8; L72; L71; |
| | | | | | | L54 |
| 2726 | QEKDEDDNLI | L41 | TLVSDIDITF | L26; L10 | VFVSNGTHWF | L8; L9; L72; L71; |
| | | | | | | L12 |
| 2727 | QEKNFTTAPA | L42 | TLVSDIDITFL | L4; L2 | VGDSAEVAV | L63; L68; L62; L5; |
| | | | | | | L67 |
| 2728 | QENWNTKH | L29 | TLYCIDGAL | L2; L4 | VGYQPYRVV | L47; L48; L64; |
| | | | | | | L69; L51 |
| 2729 | QENWNTKHS | L40 | TMCDIRQL | L64; L48 | VLEGSVAY | L1; L18; L26; L43; |
| | | | | | | L27 |
| 2730 | QESPFVMMS | L42 | TMLFTMLRK | L6; L7 | VLGSLAATV | L2; L3; L5; L4; L24 |
| 2731 | QEVFAQVK | L29 | TNDNTSRY | L1; L18 | VLNDILSR | L21; L15; L17; L6; |
| | | | | | | L43 |
| 2732 | QFAYANRNRF | L9 | TNVTIATY | L65; L29 | VLTLVYKVY | L26; L14; L43; |
| | | | | | | L27; L12 |
| 2733 | QFCNDPFL | L62 | TNYDLSVVNAR | L19; L17 | VPQEHYVRI | L49; L47; L39; |
| | | | | | | L56; L32 |
| 2734 | QFDTYNLW | L62 | TPFEIKLAKK | L19; L31 | VPVVDSYYSL | L56; L39; L32; |
| | | | | | | L22; L31 |
| 2735 | QFKHLIPLMY | L12 | TPFEIKLAKKF | L39; L31 | VPYCYDTNVL | L39; L22; L32; |
| | | | | | | L47; L56 |
| 2736 | QFKKGVQI | L48 | TPHTVLQA | L51; L50 | VPYNMRVIHF | L31; L49; L47; |
| | | | | | | L39; L22 |
| 2737 | QFNYYKKV | L48 | TPHTVLQAVGA | L50; L51 | VQIGEYTF | L26; L27; L34; |
| | | | | | | L58; L8 |
| 2738 | QFTSLEIPR | L17 | TPKGPKVK | L39; L51 | VQMAPISAM | L26; L28; L27; |
| | | | | | | L58; L61 |
| 2739 | QGLNGYTV | L48 | TPLGIDLDEW | L31; L49 | VQSTQWSLF | L26; L9; L58; L27; |
| | | | | | | L8 |
| 2740 | QGLPNNTASW | L52 | TPLIQPIGA | L50; L51 | VRNLQHRLY | L66; L64; L30; |
| | | | | | | L65; L14 |
| 2741 | QGNFGDQEL | L32 | TPSDFVRA | L50; L51 | VSKVVKVTI | L53; L55; L43; |
| | | | | | | L52; L70 |
| 2742 | QGTTLPKGFY | L14 | TPSDFVRATA | L51; L50 | VSLVKPSF | L59; L52; L43; |
| | | | | | | L54; L53 |
| 2743 | QGYKSVNI | L48 | TPVCINGL | L56; L39 | VSSPDAVTA | L53; L52; L54; |
| | | | | | | L55; L69 |
| 2744 | QHGKEDLKF | L34 | TPVCINGLM | L32; L31 | VSTQEFRYM | L55; L52; L73; |
| | | | | | | L60; L70 |
| 2745 | QHLKDGTCGL | L34 | TPVHVMSKH | L32; L49 | VSWNLREM | L59; L70; L55; |
| | | | | | | L48; L43 |
| 2746 | QHMVVKAA | L28 | TPVYSFLPGV | L51; L50 | VTANVNAL | L70; L67; L60; |
| | | | | | | L59; L61 |
| 2747 | QHQPYVVD | L28 | TQALPQRQK | L13; L7 | VTCGTTTL | L60; L70; L68; |
| | | | | | | L63; L57 |
| 2748 | QHQPYVVDD | L28 | TQDLFLPF | L68; L62 | VTIDYTEI | L55; L67; L70; |
| | | | | | | L59; L53 |
| 2749 | QIAQFAPSA | L28 | TQHQPYVVD | L26; L44 | VTLAILTALR | L21; L15; L7; L19; |
| | | | | | | L17 |
| 2750 | QIGYYRRATR | L21 | TQMNLKYAI | L24; L34 | VTPSGTWL | L57; L63; L72; |
| | | | | | | L71; L60 |
| 2751 | QIPFAMQM | L57 | TRAKVGILCI | L34; L35 | VTSSIVITS | L20; L53; L70; |
| | | | | | | L19; L69 |
| 2752 | QITISSFKW | L54 | TRALTAESH | L35; L30 | VTVKNGSIHLY | L14; L18; L52; |
| | | | | | | L11; L1 |
| 2753 | QIYKTPPI | L48 | TREVGFVVP | L35; L42 | VTYVPAQEK | L13; L7; L43; L6; |
| | | | | | | L91 |
| 2754 | QIYKTPPIK | L6 | TRFASVYA | L35; L65 | VVDSYYSLLM | L1; L18; L63; L5; |
| | | | | | | L68 |
| 2755 | QKAAITIL | L44 | TRFQTLLA | L35; L25 | VVDYGARF | L63; L62; L68; L1; |
| | | | | | | L54 |
| 2756 | QKFNGLTV | L48 | TRGATVVI | L35; L65 | VVGDIILKP | L12; L24; L6; L11; |
| | | | | | | L7 |
| 2757 | QKLIANQF | L27 | TRNPANNAAI | L35; L34 | VVGEGSEGL | L16; L54; L2; L67; |
| | | | | | | L56 |
| 2758 | QKRTATKAY | L27 | TRTQLPPAY | L66; L30 | VVISSDVLV | L20; L5; L2; L73; |
| | | | | | | L58 |
| 2759 | QKTYERHSL | L44 | TSAFVETVK | L19; L7 | VVQEGVLTAV | L3; L2; L4; L5; L51 |
| 2760 | QKVGMQKY | L27 | TSDLATNN | L68; L1 | VVQTIEVNSF | L16; L26; L52; L9; |
| | | | | | | L10 |
| 2761 | QLDEEQPME | L5 | TSDYYQLYS | L1; L18 | VVSKVVKV | L24; L48; L73; |
| | | | | | | L20; L63 |
| 2762 | QLDFIDTKR | L21 | TSEAVEAPL | L63; L68 | VVSTGYHFR | L21; L15; L19; L7; |
| | | | | | | L17 |
| 2763 | QLGGLHLL | L74 | TSEDMLNPNY | L1; L18 | VVSTTTNIVTR | L19; L7; L21; L15; |
| | | | | | | L17 |
| 2764 | QLGGLHLLI | L24 | TSGDGTTSP | L43; L65 | VVVLSFELL | L74; L67; L60; |
| | | | | | | L20; L31 |
| 2765 | QLHNDILL | L74 | TSKETLYCI | L55; L53 | VVYRAFDIY | L14; L12; L26; |
| | | | | | | L18; L6 |
| 2766 | QLHNDILLAK | L6 | TSLEIPRRN | L55; L52 | VYADSFVI | L9; L8; L66; L72; |
| | | | | | | L71 |
| 2767 | QLIRAAEIR | L21 | TSLSGFKL | L55; L54 | VYGDFSHSQ | L9; L66; L8; L72; |
| | | | | | | L71 |
| 2768 | QLPAPRTLLTK | L6 | TSNSFDVLK | L7; L19 | VYGDFSHSQL | L8; L9; L66; L71; |
| | | | | | | L72 |
| 2769 | QLPETYFTQS | L5 | TSRTLSYYK | L13; L7 | VYSHLLLV | L62; L64; L73; |
| | | | | | | L48; L9 |
| 2770 | QLPPAYTNS | L5 | TSRYWEPEFY | L43; L1 | VYSRVKNL | L72; L71; L9; L66; |
| | | | | | | L62 |
| 2771 | QLPQGTTLPK | L6 | TTAAKLMVVI | L20; L10 | WEPEFYEAM | L29; L36; L57; |
| | | | | | | L37; L33 |
| 2772 | QLRARSVSPK | L6 | TTADIVVFDE | L20; L19 | WFLAYILF | L71; L9; L62; L72; |
| | | | | | | L8 |
| 2773 | QLREPMLQSA | L3 | TTDNTFVSG | L1; L18 | WMESEFRVY | L27; L1; L18; L14; |
| | | | | | | L26 |
| 2774 | QLRVESSSK | L13 | TTDPSFLG | L1; L18 | WNLVIGFLF | L12; L9; L8; L52; |
| | | | | | | L40 |
| 2775 | QLSLPVLQVR | L21 | TTEAFEKM | L18; L1 | WTAGAAAY | L18; L14; L1; L59; |
| | | | | | | L54 |
| 2776 | QLTSQWLTNI | L3 | TTIAKNTV | L20; L48 | WVLNNDYYR | L17; L15; L21; |
| | | | | | | L19; L7 |
| 2777 | QMAPISAM | L28 | TTITVNVLA | L20; L19 | YAADPAMH | L59; L43; L69; |
| | | | | | | L70; L74 |
| 2778 | QMNLKYAI | L48 | TTITVNVLAWL | L1; L18 | YACWHHSIGF | L58; L43; L49; |
| | | | Y | | | L59; L60 |
| 2779 | QMYKQARS | L48 | TTIVNGVRRSF | L52; L10 | YADSFVIR | L67; L63; L68; |
| | | | | | | L17; L74 |
| 2780 | QNCVLKLKV | L73 | TTLKGVEAV | L20; L4 | YANLGERV | L50; L47; L48; |
| | | | | | | L58; L70 |
| 2781 | QNGMNGRTI | L55 | TTLNDFNLV | L20; L73 | YASALWEI | L59; L47; L70; |
| | | | | | | L69; L67 |
| 2782 | QPIDLVPNQ | L31 | TTLPKGFY | L14; L1 | YASQGLVAS | L59; L50; L31; |
| | | | | | | L67; L32 |
| 2783 | QPIDLVPNQPY | L31 | TTNGDFLHFL | L20; L53 | YDPLQPEL | L33; L57; L66; |
| | | | | | | L59; L67 |
| 2784 | QPIGALDI | L47 | TTPGSGVPV | L57; L20 | YEAMYTPHTV | L45; L38; L46; |
| | | | | | | L37; L36 |
| 2785 | QPYPNASFD | L22 | TTPGSGVPVV | L20; L57 | YFLQSINFV | L72; L47; L71; L4; |
| | | | | | | L62 |
| 2786 | QPYRVVVLS | L47 | TTRTQLPPAY | L52; L14 | YGQQFGPTY | L59; L26; L14; |
| | | | | | | L27; L12 |
| 2787 | QPYVFIKR | L47 | TTTIKPVTYK | L6; L7 | YHFRELGVV | L28; L35; L46; L3; |
| | | | | | | L34 |
| 2788 | QPYVFIKRS | L48 | TTTIQTIVEV | L20; L73 | YIATNGPLKV | L3; L2; L73; L58; |
| | | | | | | L4 |
| 2789 | QQESPFVMM | L28 | TTTNIVTRC | L20; L19 | YILPSIISN | L4; L60; L43; L61; |
| | | | | | | L6 |
| 2790 | QQGQTVTK | L48 | TTVASLINTL | L10; L20 | YINVFAFPF | L59; L43; L12; |
| | | | | | | L58; L60 |
| 2791 | QQIELKFN | L48 | TTVMFLAR | L17; L19 | YKGPITDVF | L66; L27; L9; L44; |
| | | | | | | L58 |
| 2792 | QQLPETYF | L27 | TVAGVSICS | L19; L20 | YKIEELFYSY | L27; L12; L66; |
| | | | | | | L31; L10 |
| 2793 | QQQGQTVTK | L7 | TVAGVSICST | L19; L20 | YKLEGYAF | L27; L66; L71; |
| | | | | | | L59; L72 |
| 2794 | QQTTLKGV | L48 | TVEEAKTVLKK | L6; L7 | YLASGGQPI | L24; L2; L3; L43; |
| | | | | | | L49 |
| 2795 | QQTVTLLP | L48 | TVFPPTSFG | L20; L6 | YLAVFDKNL | L2; L4; L3; L74; |
| | | | | | | L64 |
| 2796 | QQWGFTGNL | L44 | TVIWDYKR | L19; L17 | YLCFLAFL | L62; L74; L59; |
| | | | | | | L57; L68 |
| 2797 | QRGGSYTN | L65 | TVIWDYKRD | L10; L11 | YLDGADVTK | L5; L2; L68; L63; |
| | | | | | | L1 |
| 2798 | QRKYKGIKI | L30 | TVKSVGKF | L10; L11 | YLITPVHV | L2; L3; L4; L5; L62 |
| 2799 | QRLTKYTM | L66 | TVKSVGKFC | L13; L39 | YNSASFSTF | L9; L34; L49; L58; |
| | | | | | | L27 |
| 2800 | QRNFYEPQII | L30 | TVREVLSD | L65; L43 | YPDPSRILG | L32; L49; L22; |
| | | | | | | L31; L1 |
| 2801 | QRQKKQQTV | L35 | TVSCLPFTI | L20; L24 | YPQVNGLTSI | L22; L47; L32; |
| | | | | | | L50; L39 |
| 2802 | QSAPHGVVFLH | L52 | TVTLLPAA | L51; L50 | YQGKPLEF | L58; L27; L43; |
| | | | | | | L26; L59 |
| 2803 | QSCTQHQPY | L18 | TVYDDGAR | L17; L19 | YRENGIGVT | L65; L35; L66; |
| | | | | | | L64; L25 |
| 2804 | QSFLNGFAV | L48 | TWFHAIHV | L48; L47 | YRGTTTYKL | L66; L65; L35; |
| | | | | | | L64; L34 |
| 2805 | QSRNLQEFK | L13 | TYACWHHSIGF | L9; L8 | YRIGNYKL | L35; L25; L66; |
| | | | | | | L34; L64 |
| 2806 | QTNSPRRA | L55 | TYASQGLVASI | L9; L8 | YSKWYIRVG | L70; L55; L65; |
| | | | | | | L43; L69 |
| 2807 | QTPFEIKLAK | L7 | TYCTGSIPC | L71; L72 | YSLFDMSKF | L53; L52; L54; |
| | | | | | | L58; L60 |
| 2808 | QTQTNSPRR | L21 | TYERHSLSHF | L8; L9 | YSLLMPILTL | L60; L67; L61; |
| | | | | | | L73; L59 |
| 2809 | QTRAGCLI | L55 | TYFTQSRNLQEF | L8; L9 | YSPIFLIVA | L50; L69; L46; |
| | | | | | | L59; L64 |
| 2810 | QTSITSAV | L48 | TYGQQFGPTY | L12; L14 | YSRYRIGNY | L43; L58; L1; L14; |
| | | | | | | L10 |
| 2811 | QTTPGSGVPV | L20 | TYHPNCVNC | L8; L72 | YTDFATSAC | L68; L1; L18; L63; |
| | | | | | | L60 |
| 2812 | QTTTIQTIV | L20 | TYICGFIQQK | L17; L13 | YWEPEFYEA | L38; L50; L46; |
| | | | | | | L42; L8 |
| 2813 | QTVKPGNFNK | L7 | TYICGFIQQKL | L8; L9 | YYLFDESGEF | L9; L71; L72; L8; |
| | | | | | | L12 |
| 2814 | QTYHPNCV | L48 | TYKNTCDGTTF | L8; L9 | YYLGTGPEA | L71; L72; L8; L9; |
| | | | | | | L50 |
| 2815 | QVAVLYQDV | L20 | TYKPNTWCI | L8; L9 | YYPDKVFR | L71; L66; L72; |
| | | | | | | L62; L17 |
| 2816 | QVHGNAHVA | L51 | TYKPNTWCIR | L15; L17 | YYPDKVFRSS | L72; L71; L66; L9; |
| | | | | | | L8 |
| 2817 | QVNGLTSI | L48 | TYLDGADV | L71; L72 | YYRYNLPTM | L66; L71; L9; L72; |
| | | | | | | L59 |
| 2818 | QVTIAEILL | L74 | TYLDGADVTKI | L8; L9 | YYVWKSYVH | L66; L71; L8; L72; |
| | | | | | | L9 |
| 2819 | QVVNVVTTKI | L20 | TYNCCDDDYF | L8; L9 | AADPAMHAA | L68; L67; L61; |
| | | | | | | L63; L42; L38 |
| 2820 | QWLPTGTLLV | L8 | TYNLWNTFTRL | L8; L9 | AARYMRSL | L59; L70; L55; |
| | | | | | | L61; L43; L60 |
| 2821 | QWNLVIGFLFL | L40 | TYPSLETIQI | L9; L8 | ACPDGVKHV | L57; L64; L5; L3; |
| | | | | | | L24; L69 |
| 2822 | QYLNTLTLAV | L71 | TYVPAQEK | L72; L71 | AEYHNESGL | L37; L36; L45; |
| | | | | | | L41; L40; L33 |
| 2823 | RAFGEYSH | L54 | TYVPAQEKN | L8; L9 | AFGGCVFSY | L12; L65; L14; |
| | | | | | | L72; L71; L26 |
| 2824 | RAGEAANFCAL | L67 | TYVPAQEKNF | L8; L9 | AFKLNIKLL | L13; L62; L64; |
| | | | | | | L39; L9; L72 |
| 2825 | RAKHYVYI | L70 | TYVTQQLI | L9; L8 | AFLLFLVLI | L9; L8; L48; L62; |
| | | | | | | L40; L41 |
| 2826 | RALTAESH | L54 | TYVTQQLIR | L17; L15 | AFPFTIYSLL | L72; L9; L71; L62; |
| | | | | | | L57; L8 |
| 2827 | RALTGIAV | L50 | VAAFHQEC | L70; L59 | AGDSGFAAY | L18; L14; L1; L27; |
| | | | | | | L12; L43 |
| 2828 | RARAGEAA | L51 | VAAFHQECSL | L67; L61 | AIKITEHSW | L16; L14; L26; |
| | | | | | | L54; L10; L52 |
| 2829 | RARAGEAANF | L52 | VAAGLEAPFLY | L12; L31 | AIVFITLCF | L26; L12; L58; |
| | | | | | | L60; L43; L41 |
| 2830 | RARSVSPK | L13 | VAAIFYLIT | L69; L43 | APYIVGDVV | L51; L50; L47; |
| | | | | | | L39; L22; L24 |
| 2831 | RCILHCANF | L33 | VAALTNNV | L47; L70 | AQFAPSASA | L46; L28; L51; |
| | | | | | | L27; L44; L42 |
| 2832 | RCLAVHEC | L33 | VADAVIKT | L63; L68 | ARSVSPKLF | L30; L66; L34; |
| | | | | | | L64; L35; L9 |
| 2833 | RCSFYEDF | L33 | VAFELWAK | L59; L7 | ARYMRSLKV | L25; L64; L30; |
| | | | | | | L73; L24; L35 |
| 2834 | RCSFYEDFLEY | L18 | VAFNTLLFLM | L31; L69 | ATNNLVVMAY | L14; L16; L18; |
| | | | | | | L12; L10; L6 |
| 2835 | RDAAMQRK | L33 | VAITRAKVG | L69; L70 | AVFDKNLYD | L6; L7; L12; L13; |
| | | | | | | L11; L16 |
| 2836 | RDAPAHIS | L33 | VATVQSKM | L70; L63 | AVILRGHLR | L15; L21; L19; |
| | | | | | | L17; L7; L6 |
| 2837 | RDAPAHIST | L33 | VAYESLRP | L43; L47 | AVVYRGTTTY | L26; L10; L18; |
| | | | | | | L12; L14; L11 |
| 2838 | RDAPAHISTI | L33 | VAYRKVLLRK | L6; L7 | AWQPGVAM | L71; L72; L28; |
| | | | | | | L33; L62; L59 |
| 2839 | RDIADTTDA | L46 | VAYSNNSIA | L69; L70 | AYILFTRFFY | L12; L14; L72; |
| | | | | | | L71; L41; L40 |
| 2840 | RDISTEIY | L29 | VCNLLLLF | L62; L59 | CANDPVGFTL | L61; L60; L68; |
| | | | | | | L32; L67; L31 |
| 2841 | RDLICAQK | L33 | VCNLLLLFV | L73; L64 | CFANKHADF | L8; L71; L72; L9; |
| | | | | | | L62; L12 |
| 2842 | RDPQTLEI | L33 | VCSVIDLLL | L73; L34 | CFLAFLLF | L9; L8; L12; L71; |
| | | | | | | L72; L62 |
| 2843 | RDVDTDFV | L33 | VCVSTSGRW | L49; L54 | CFLAFLLFL | L71; L8; L72; L73; |
| | | | | | | L9; L62 |
| 2844 | REAVGTNLPLQL | L36 | VDIAANTV | L48; L33 | CKDGHVETF | L27; L34; L62; |
| | | | | | | L63; L49; L68 |
| 2845 | REETGLLMPL | L36 | VDLPIGINI | L8; L9 | CRSKNPLLY | L66; L30; L64; |
| | | | | | | L12; L65; L18 |
| 2846 | REGYLNST | L45 | VDMSMTYGQQF | L8; L9 | CSAYTVEL | L59; L70; L67; |
| | | | | | | L61; L74; L60 |
| 2847 | RELHLSWEVG | L36 | VDTPNNTDF | L9; L8 | CTDDNALAYY | L18; L1; L11; L14; |
| | | | | | | L12; L58 |
| 2848 | RELKVTFFPDL | L36 | VDTVSALVY | L12; L18 | CTNYMPYFF | L53; L62; L55; |
| | | | | | | L54; L16; L58 |
| 2849 | RELMRELN | L33 | VDYGARFYF | L9; L12 | CVADYSVL | L67; L61; L74; |
| | | | | | | L59; L10; L60 |
| 2850 | RELMRELNG | L33 | VDYGARFYFY | L14; L12 | CYFGLFCLL | L9; L8; L64; L62; |
| | | | | | | L66; L13 |
| 2851 | REMLAHAE | L33 | VEAEVQIDRLI | L41; L40 | CYGVSPTKL | L62; L9; L66; L72; |
| | | | | | | L8; L71 |
| 2852 | REMLAHAEE | L33 | VEGCHATREA | L38; L46 | DAQSFLNGF | L11; L49; L10; |
| | | | | | | L58; L31; L47 |
| 2853 | RENNRVVISS | L38 | VEGFNCYFP | L38; L42 | DEPEEHVQI | L45; L36; L41; |
| | | | | | | L25; L29; L37 |
| 2854 | REQIDGYV | L33 | VELKHFFF | L33; L29 | DFVNEFYAY | L12; L10; L29; |
| | | | | | | L11; L71; L72 |
| 2855 | REQIDGYVMH | L38 | VELVAELEGI | L41; L36 | DGWEIVKF | L29; L47; L23; |
| | | | | | | L49; L25; L11 |
| 2856 | REVLSDRELH | L36 | VENPHLMGWD | L41; L29 | DHVDILGPL | L35; L20; L34; |
| | | | Y | | | L11; L28; L10 |
| 2857 | REVRTIKVFT | L36 | VESDDYIAT | L38; L36 | DLSPRWYFY | L12; L1; L23; L11; |
| | | | | | | L18; L14 |
| 2858 | RFDNPVLP | L62 | VETKAIVSTI | L45; L36 | DPYEDFQEN | L49; L47; L31; |
| | | | | | | L25; L32; L10 |
| 2859 | RFDTRVLS | L62 | VETKDVVECL | L36; L37 | DRYPANSI | L25; L47; L35; |
| | | | | | | L28; L64; L66 |
| 2860 | RFDTRVLSNL | L62 | VETSNSFDVL | L36; L45 | DSLDTYPSL | L25; L61; L60; |
| | | | | | | L67; L10; L74 |
| 2861 | RFFYVLGLAA | L51 | VEYCPIFF | L29; L33 | DTLKEILVTY | L11; L10; L52; |
| | | | | | | L53; L14; L16 |
| 2862 | RFLYIIKLIFL | L8 | VFDEISMAT | L62; L68 | DVTDVTQL | L74; L11; L20; |
| | | | | | | L25; L10; L23 |
| 2863 | RFPKSDGTGTI | L8 | VFDGKSKC | L62; L63 | DVVIGIVNN | L11; L19; L17; |
| | | | | | | L49; L10; L20 |
| 2864 | RFPNITNLCPF | L8 | VFFDGRVDG | L71; L72 | EAFEYYHTT | L10; L49; L11; |
| | | | | | | L20; L69; L32 |
| 2865 | RFQTLLALHR | L15 | VFLGIITT | L71; L72 | EAMYTPHTV | L70; L20; L47; |
| | | | | | | L32; L69; L50 |
| 2866 | RFQTLLALHRSY | IL14 | VFMCVEYC | L71; L72 | ECVRGTTVL | L35; L10; L32; |
| | | | | | | L11; L34; L8 |
| 2867 | RGATVVIGT | L44 | VFPPTSFGP | L8; L9 | EEAARYMRSL | L40; L41; L42; |
| | | | | | | L10; L25; L37 |
| 2868 | RGDEVRQIA | L46 | VFQSASKII | L72; L9 | EEHFIETISL | L40; L37; L41; |
| | | | | | | L42; L36; L29 |
| 2869 | RGGDGKMKD | L65 | VFQTRAGC | L71; L72 | EEVKPFITE | L29; L46; L42; |
| | | | | | | L41; L37; L45 |
| 2870 | RGIVFMCV | L48 | VFYKENSY | L71; L72 | EIDPKLDNY | L1; L18; L11; L10; |
| | | | | | | L14; L12 |
| 2871 | RGTAVMSLK | L13 | VGDVVQEGV | L63; L5 | EQIDGYVMH | L11; L24; L43; |
| | | | | | | L34; L10; L35 |
| 2872 | RGTTTYKL | L54 | VGDVVQEGVL | L68; L63 | ETKAIVSTI | L10; L20; L11; |
| | | | | | | L70; L55; L17 |
| 2873 | RGVYYPDKV | L73 | VGFTLKNTV | L48; L47 | ETMSYLFQH | L11; L17; L20; L7; |
| | | | | | | L10; L19 |
| 2874 | RHFDEGNCD | L34 | VGFVVPGL | L48; L47 | EVFAQVKQI | L20; L10; L48; |
| | | | | | | L11; L19; L24 |
| 2875 | RHINAQVA | L28 | VGGNYNYLY | L14; L12 | EVLTEEVVL | L20; L32; L44; |
| | | | | | | L10; L31; L23 |
| 2876 | RIAGHHLGRC | L14 | VGLMWLSYF | L9; L8 | EVVENPTIQK | L19; L11; L7; L20; |
| | | | | | | L17; L6 |
| 2877 | RIDGDMVPH | L6 | VGVALLAV | L48; L47 | FAVSKGFF | L58; L74; L59; |
| | | | | | | L60; L70; L49 |
| 2878 | RIDKVLNEKC | L18 | VGYQPYRV | L48; L47 | FAYTKRNV | L47; L70; L64; |
| | | | | | | L69; L58; L59 |
| 2879 | RIIMRLWL | L73 | VHTANKWDL | L35; L34 | FDNPVLPF | L33; L59; L43; |
| | | | | | | L71; L72; L66 |
| 2880 | RIIMRLWLC | L21 | VIGIVNNTVY | L43; L26 | FEYVSQPFL | L36; L45; L37; |
| | | | | | | L61; L74; L60 |
| 2881 | RIIMRLWLCWK | L6 | VILRGHLR | L21; L15 | FFKEGSSVEL | L62; L71; L72; |
| | | | | | | L43; L67; L8 |
| 2882 | RIKASMPTT | L13 | VIPTITQM | L57; L63 | FFTLLLQL | L62; L59; L72; |
| | | | | | | L71; L74; L67 |
| 2883 | RIKASMPTTI | L3 | VIPTITQMNL | L57; L5 | FIKGLNNL | L58; L74; L59; |
| | | | | | | L60; L23; L67 |
| 2884 | RIKASMPTTIAK | L6 | VKCTSVVL | L44; L66 | FKDQVILL | L74; L62; L68; |
| | | | | | | L63; L66; L64 |
| 2885 | RIMASLVLA | L6 | VKPSFYVY | L66; L27 | FLAFLLFLVL | L67; L74; L2; L3; |
| | | | | | | L61; L20 |
| 2886 | RINWITGGI | L24 | VLAAECTIFK | L6; L7 | FLAFVVFLLV | L2; L20; L3; L12; |
| | | | | | | L4; L5 |
| 2887 | RIRGGDGK | L13 | VLALLSDL | L74; L23 | FLFLTWICLL | L74; L2; L4; L3; |
| | | | | | | L64; L5 |
| 2888 | RIRGGDGKM | L14 | VLDMCASLKEL | L68; L5 | FLGIITTV | L3; L2; L23; L48; |
| | | | | | | L42; L4 |
| 2889 | RIRGGDGKMK | L13 | VLDWLEEK | L68; L1 | FLLFLVLIM | L2; L74; L4; L12; |
| | | | | | | L69; L67 |
| 2890 | RKIFVDGVP | L44 | VLHSTQDL | L57; L43 | FLLNKEMYL | L2; L74; L4; L5; |
| | | | | | | L23; L61 |
| 2891 | RKLDNDALN | L44 | VLHSYFTSD | L43; L57 | FLPGVYSVI | L57; L5; L3; L10; |
| | | | | | | L64; L2 |
| 2892 | RKLHDELTG | L44 | VLHSYFTSDYY | L14; L12 | FLYENAFLPF | L59; L12; L6; L2; |
| | | | | | | L5; L10 |
| 2893 | RKMAFPSGK | L6 | VLIMLIIFWF | L12; L9 | FLYIIKLI | L48; L47; L64; |
| | | | | | | L23; L24; L3 |
| 2894 | RKRISNCVADY | L27 | VLKKCKSAFY | L43; L14 | FNEKTHVQL | L63; L32; L68; |
| | | | | | | L62; L74; L34 |
| 2895 | RKSNHNFL | L44 | VLKLKVDTA | L3; L23 | FPFTIYSLLL | L31; L32; L47; |
| | | | | | | L22; L50; L49 |
| 2896 | RKSNLKPFER | L21 | VLLFLAFV | L4; L2 | FPPTSFGPL | L57; L22; L66; |
| | | | | | | L47; L56; L72 |
| 2897 | RKVPTDNY | L27 | VLLFLAFVVF | L9; L12 | FPREGVFV | L50; L47; L51; |
| | | | | | | L23; L22; L39 |
| 2898 | RKVPTDNYI | L44 | VLLPLTQYNR | L15; L21 | FPRGQGVPI | L50; L22; L49; |
| | | | | | | L47; L39; L51 |
| 2899 | RKVPTDNYITT | L44 | VLLPLTQYNRY | L12; L14 | FQSASKIITL | L44; L34; L28; |
| | | | | | | L35; L69; L58 |
| 2900 | RKVPTDNYITTY | L27 | VLLSMQGAV | L2; L4 | FSKQLQQSM | L58; L70; L43; |
| | | | | | | L55; L69; L53 |
| 2901 | RKYKGIKIQE | L27 | VLLSVLQQLRV | L4; L2 | FSTFEEAAL | L61; L60; L67; |
| | | | | | | L68; L74; L59 |
| 2902 | RLCAYCCNI | L24 | VLNEKCSA | L23; L43 | FSTGVNLV | L70; L73; L64; |
| | | | | | | L48; L69; L58 |
| 2903 | RLDKVEAEVQ | L18 | VLNNDYYR | L21; L15 | FTEQPIDL | L63; L60; L68; |
| | | | | | | L62; L1; L61 |
| 2904 | RLFARTRSMW | L16 | VLPPLLTDE | L5; L57 | FTRFFYVL | L59; L60; L67; |
| | | | | | | L70; L61; L66 |
| 2905 | RLIDAMMFT | L2 | VLPPLLTDEM | L5; L57 | FVDDIVKT | L63; L68; L62; L5; |
| | | | | | | L1; L74 |
| 2906 | RLISMMGFKMNY | L18 | VLPQLEQPYV | L5; L2 | FVENPDIL | L67; L62; L74; |
| | | | | | | L63; L68; L60 |
| 2907 | RLITGRLQ | L55 | VLQLPQGTTL | L57; L26 | FVVEVVDKYF | L11; L12; L60; |
| | | | | | | L52; L10; L58 |
| 2908 | RLKLFDRYFK | L13 | VLQSGFRKM | L14; L13 | FVVKRHTF | L58; L59; L54; |
| | | | | | | L60; L23; L26 |
| 2909 | RLMKTIGPDMF | L16 | VLQVRDVLVR | L15; L21 | FYVLGLAAI | L72; L71; L9; L59; |
| | | | | | | L62; L50 |
| 2910 | RLTKYTMADLV | L18 | VLRQWLPTG | L3; L4 | FYYVWKSYV | L64; L71; L62; |
| | Y | | | | | L72; L47; L69 |
| 2911 | RLWLCWKCRSK | L6 | VLSFCAFAV | L4; L2 | GAEHVNNSY | L18; L31; L12; L1; |
| | | | | | | L14; L49 |
| 2912 | RMNSRNYIA | L38 | VLSGHNLAK | L6; L7 | GAKLKALNL | L23; L70; L74; |
| | | | | | | L60; L58; L13 |
| 2913 | RNFYEPQI | L48 | VLTLVYKV | L48; L24 | GECPNFVFP | L46; L42; L38; |
| | | | | | | L37; L45; L36 |
| 2914 | RNPANNAAI | L57 | VLTLVYKVYY | L12; L14 | GEFKLASHM | L36; L29; L37; |
| | | | | | | L45; L41; L40 |
| 2915 | RNPAWRKAV | L57 | VLYQPPQTSI | L6; L3 | GFGDSVEEV | L72; L2; L24; L3; |
| | | | | | | L62; L71 |
| 2916 | RNRATRVEC | L13 | VMFLARGIVF | L26; L9 | GFMGRIRSV | L3; L64; L72; L48; |
| | | | | | | L45; L71 |
| 2917 | RNRDVDTD | L65 | VMMSAPPAQY | L14; L12 | GIMMNVAKY | L26; L12; L14; |
| | | | | | | L10; L11; L18 |
| 2918 | RNRFLYIIK | L13 | VMYASAVV | L48; L47 | GLPWNVVRI | L24; L5; L3; L2; |
| | | | | | | L4; L64 |
| 2919 | RNTNPIQLSSY | L14 | VNLKQLPFF | L9; L8 | GLVAEWFLAY | L12; L6; L26; L10; |
| | | | | | | L27; L18 |
| 2920 | RNVIPTITQM | L65 | VNQNAQAL | L71; L72 | GRLQSLQTY | L30; L27; L14; |
| | | | | | | L34; L12; L26 |
| 2921 | RPLLESELV | L51 | VPAQEKNFTTA | L51; L50 | GRTILGSAL | L30; L35; L28; |
| | | | | | | L25; L34; L66 |
| 2922 | RPNFTIKGS | L39 | VPEVKILNNL | L39; L56 | GSVGFNIDY | L26; L18; L27; |
| | | | | | | L14; L58; L7 |
| 2923 | RPQGLPNNT | L56 | VPHVGEIPVA | L51; L50 | GSYKDWSY | L55; L18; L27; |
| | | | | | | L14; L54; L26 |
| 2924 | RPQIGVVREFL | L56 | VPLHGTILT | L50; L31 | GTSTDVVYR | L7; L21; L15; L19; |
| | | | | | | L17; L6 |
| 2925 | RQCSGVTFQ | L24 | VPLNIIPLTTA | L50; L51 | GVAPGTAVLR | L19; L7; L21; L15; |
| | | | | | | L6; L17 |
| 2926 | RQFHQKLLKS | L24 | VPMEKLKTLVA | L50; L51 | GVKDCVVLHSY | L14; L10; L11; |
| | | | | | | L26; L12; L52 |
| 2927 | RQGFVDSDV | L24 | VPNQPYPNASF | L22; L31 | GVRRSFYVY | L26; L14; L10; |
| | | | | | | L12; L27; L52 |
| 2928 | RQHLKDGTC | L14 | VPQADVEWKF | L31; L49 | GVVREFLTR | L7; L19; L15; L17; |
| | | | | | | L10; L21 |
| 2929 | RQKRTATKA | L24 | VPRASANI | L47; L22 | GVYYPDKVF | L26; L27; L52; |
| | | | | | | L10; L39; L16 |
| 2930 | RQRLTKYT | L48 | VPVSIINNT | L49; L51 | GYLQPRTF | L55; L9; L72; L71; |
| | | | | | | L65; L8 |
| 2931 | RQVVNVVT | L44 | VPWDTIAN | L50; L51 | GYPNMFITR | L17; L15; L7; L9; |
| | | | | | | L27; L71 |
| 2932 | RQVVNVVTTKI | L24 | VPWDTIANYA | L51; L50 | GYQPYRVVV | L72; L13; L71; |
| | | | | | | L38; L64; L66 |
| 2933 | RQWLPTGTLLV | L24 | VPYCYDTNV | L47; L51 | GYVTHGLNL | L71; L8; L72; L9; |
| | | | | | | L66; L65 |
| 2934 | RRAFGEYSHV | L30 | VQIDRLITGR | L21; L15 | HADFDTWF | L63; L68; L32; |
| | | | | | | L74; L62; L49 |
| 2935 | RRARSVASQ | L30 | VQIPTTCAN | L43; L26 | HECFVKRVD | L37; L38; L65; |
| | | | | | | L42; L45; L46 |
| 2936 | RRGPEQTQ | L65 | VQLSLPVLQV | L4; L24 | HEVLLAPL | L29; L37; L25; |
| | | | | | | L33; L36; L45 |
| 2937 | RRGPEQTQGNF | L30 | VQLTSQWL | L25; L44 | HFLPRVFSA | L50; L8; L72; L71; |
| | | | | | | L38; L46 |
| 2938 | RRIRGGDGK | L30 | VQPTESIVR | L21; L15 | HFVCNLLLL | L8; L62; L72; L73; |
| | | | | | | L9; L71 |
| 2939 | RRIRGGDGKM | L30 | VQSTQWSL | L44; L35 | HHWLLLTIL | L34; L35; L28; |
| | | | | | | L44; L25; L31 |
| 2940 | RRIRGGDGKMK | L30 | VRDLPQGF | L62; L63 | HQSDIEVTG | L44; L24; L26; |
| | | | | | | L34; L35; L28 |
| 2941 | RRLISMMGFK | L30 | VRITGLYPTL | L66; L30 | HVASCDAIMTR | L19; L21; L17; |
| | | | | | | L11; L15; L7 |
| 2942 | RRLISMMGFKM | L30 | VRNLQHRL | L66; L65 | HVETFYPKL | L32; L63; L11; |
| | | | | | | L49; L13; L5 |
| 2943 | RRNVATLQA | L30 | VRQALLKTV | L64; L30 | HVTFFIYNK | L7; L13; L19; L20; |
| | | | | | | L6; L17 |
| 2944 | RRPQGLPNNT | L30 | VRRSFYVY | L66; L65 | HVVGPNVNK | L19; L7; L13; L6; |
| | | | | | | L20; L17 |
| 2945 | RRSFYVYA | L65 | VSDADSTLIG | L18; L1 | HWLLLTIL | L25; L23; L62; |
| | | | | | | L33; L59; L48 |
| 2946 | RRVVFNGV | L30 | VSDIDYVPLK | L1; L18 | HYVRITGL | L72; L65; L66; |
| | | | | | | L71; L25; L8 |
| 2947 | RRVWTLMN | L65 | VSDVGDSA | L1; L68 | IAIAMACLV | L47; L69; L70; |
| | | | | | | L58; L73; L64 |
| 2948 | RSDARTAPH | L18 | VSEETGTLIV | L18; L1 | IAMSAFAMM | L61; L59; L60; |
| | | | | | | L58; L70; L31 |
| 2949 | RSDARTAPHGH | L18 | VSEEVVEN | L63; L68 | IELSLIDFY | L40; L41; L29; |
| | | | | | | L12; L14; L36 |
| 2950 | RSDVLLPLTQ | L18 | VSFLAHIQ | L55; L48 | IEVQGYKSV | L45; L46; L38; |
| | | | | | | L37; L29; L36 |
| 2951 | RSEAGVCV | L18 | VSFSTFEEA | L43; L50 | IEYPIIGD | L29; L33; L45; |
| | | | | | | L46; L25; L48 |
| 2952 | RSEKSYELQT | L18 | VSICSTMTNR | L19; L15 | IFLIVAAIVF | L8; L9; L71; L72; |
| | | | | | | L12; L31 |
| 2953 | RSFYVYAN | L65 | VSIINNTV | L48; L70 | IIKNLSKSL | L39; L43; L58; |
| | | | | | | L60; L26; L13 |
| 2954 | RSFYVYANG | L13 | VSIWNLDYI | L73; L55 | IIQFPNTYL | L68; L60; L61; |
| | | | | | | L74; L63; L28 |
| 2955 | RSGETLGVLV | L73 | VSKGFFKEG | L43; L13 | IKVTLVFLF | L66; L9; L27; L52; |
| | | | | | | L8; L12 |
| 2956 | RSKNPLLYDANY | L14 | VSLAIDAY | L14; L1 | ILSPLYAF | L59; L26; L23; L9; |
| | | | | | | L54; L33 |
| 2957 | RSKQRRPQ | L55 | VSPTKLNDL | L57; L64 | IPLTTAAKL | L47; L32; L31; |
| | | | | | | L39; L22; L56 |
| 2958 | RSKQRRPQG | L13 | VSSQCVNL | L74; L70 | IPVAYRKVLL | L31; L56; L22; |
| | | | | | | L32; L49; L39 |
| 2959 | RSKQRRPQGL | L55 | VSSQCVNLTTR | L19; L15 | IQLSSYSLF | L27; L9; L8; L26; |
| | | | | | | L34; L16 |
| 2960 | RSLKVPATVS | L53 | VSTTTNIV | L48; L70 | IQYIDIGNY | L26; L27; L14; |
| | | | | | | L18; L43; L11 |
| 2961 | RSQMEIDFLEL | L73 | VSVSSPDAV | L67; L73 | IRGDEVRQI | L64; L65; L34; |
| | | | | | | L66; L30; L35 |
| 2962 | RSRNSSRNS | L13 | VTDFNAIATC | L18; L1 | ISAGFSLWV | L73; L70; L69; |
| | | | | | | L58; L64; L18 |
| 2963 | RSSSRSRNS | L55 | VTDTPKGPK | L1; L7 | ISNEKQEI | L55; L48; L43; |
| | | | | | | L73; L54; L53 |
| 2964 | RSSVLHSTQ | L53 | VTDTPKGPKVK | L1; L18 | ITEEVGHTD | L53; L55; L18; |
| | | | Y | | | L49; L54; L1 |
| 2965 | RSTNSRIK | L55 | VTDVTQLYLG | L1; L18 | ITFELDERI | L20; L53; L54; |
| | | | | | | L52; L16; L34 |
| 2966 | RSTNSRIKA | L13 | VTFFIYNK | L7; L13 | ITLCFTLKR | L7; L21; L6; L17; |
| | | | | | | L15; L19 |
| 2967 | RSYLTPGD | L48 | VTFFIYNKIV | L70; L20 | ITVNVLAWLY | L12; L14; L52; |
| | | | | | | L18; L54; L1 |
| 2968 | RTFLLKYNE | L53 | VTKENDSKEGF | L52; L53 | IYLYLTFYL | L8; L9; L71; L72; |
| | | | | | | L66; L62 |
| 2969 | RTIKGTHH | L54 | VTKNSKVQI | L53; L70 | KEILVTYNC | L38; L33; L36; |
| | | | | | | L37; L42; L45 |
| 2970 | RTLETAQN | L54 | VTLADAGFIK | L7; L6 | KEITVATSR | L15; L21; L38; |
| | | | | | | L36; L37; L45 |
| 2971 | RTLETAQNSV | L16 | VTLVFLFV | L73; L47 | KEKVNINIV | L45; L37; L42; |
| | | | | | | L38; L46; L36 |
| 2972 | RTNVYLAVFDK | L7 | VTLVFLFVA | L52; L50 | KFVCDNIKF | L8; L53; L54; L9; |
| | | | | | | L52; L12 |
| 2973 | RTRSMWSFN | L13 | VTMPLGYV | L73; L70 | KFYGGWHNM | L71; L72; L14; L8; |
| | | | | | | L13; L16 |
| 2974 | RTTNGDFLHFL | L53 | VTQLYLGGMSY | L1; L18 | KGAGGHSY | L14; L55; L26; |
| | | | Y | | | L27; L54; L18 |
| 2975 | RTVYDDGARRV | L52 | VTSAMQTML | L73; L70 | KGDYGDAVVY | L18; L14; L27; |
| | W | | | | | L65; L54; L1 |
| 2976 | RVAGDSGFA | L14 | VTTEILPVSM | L52; L20 | KHAFHTPAF | L28; L27; L34; |
| | | | | | | L33; L35; L9 |
| 2977 | RVAGDSGFAA | L51 | VTTFDSEY | L1; L18 | KKADETQAL | L44; L27; L66; |
| | | | | | | L16; L64; L24 |
| 2978 | RVCGVSAA | L51 | VTVKNGSIHL | L53; L60 | KLNDLCFTNV | L5; L3; L2; L4; |
| | | | | | | L13; L21 |
| 2979 | RVCTNYMPYFF | L16 | VTVYSHLLLV | L73; L20 | KLNEEIAII | L3; L4; L16; L24; |
| | | | | | | L2; L5 |
| 2980 | RVCVDTVRTNVY | L18 | VTYKLDGVVC | L53; L52 | KLPDDFTGC | L5; L57; L6; L2; |
| | | | | | | L16; L26 |
| 2981 | RVDFCGKGYH | L18 | VVADAVIK | L7; L19 | KLVLSVNPY | L26; L16; L6; L27; |
| | | | | | | L18; L14 |
| 2982 | RVDFCGKGYHL | L18 | VVAFNTLL | L74; L68 | KMKDLSPRW | L54; L52; L16; |
| | M | | | | | L14; L13; L53 |
| 2983 | RVDWTIEYP | L18 | VVCFNSTY | L26; L18 | KMNYQVNGY | L26; L16; L14; |
| | | | | | | L27; L18; L6 |
| 2984 | RVEAFEYYH | L18 | VVCTEIDPK | L7; L6 | KNGSIHLYF | L73; L65; L12; L9; |
| | | | | | | L54; L8 |
| 2985 | RVECFDKF | L18 | VVDADSKI | L63; L68 | KNIDGYFKI | L8; L24; L73; L55; |
| | | | | | | L16; L45 |
| 2986 | RVECFDKFK | L13 | VVDGCNSST | L68; L63 | KPASRELKV | L73; L51; L24; |
| | | | | | | L39; L22; L47 |
| 2987 | RVECTTIVN | L18 | VVDYGARFYFY | L1; L18 | KPNTWCIRCL | L39; L56; L32; |
| | | | | | | L55; L22; L65 |
| 2988 | RVIHFGAGSD | L14 | VVEVVDKY | L1; L18 | KPYIKWDL | L23; L56; L25; |
| | | | | | | L39; L65; L22 |
| 2989 | RVKNLNSSRV | L13 | VVEVVDKYFDC | L18; L1 | KRFDNPVL | L65; L66; L25; |
| | | | Y | | | L35; L64; L28 |
| 2990 | RVLGLKTLA | L24 | VVFDEISMATNY | L14; L12 | KSAAEASKK | L13; L7; L54; L55; |
| | | | | | | L18; L6 |
| 2991 | RVLGLKTLAT | L6 | VVKVTIDY | L43; L26 | KSAQCFKMF | L53; L52; L55; |
| | | | | | | L54; L16; L73 |
| 2992 | RVTKNSKV | L24 | VVLLILMTA | L50; L51 | KSILSPLY | L55; L54; L18; |
| | | | | | | L52; L1; L14 |
| 2993 | RVVVLSFELL | L20 | VVLLSVLQQ | L6; L7 | KSLTENKY | L55; L54; L14; |
| | | | | | | L18; L52; L53 |
| 2994 | RVWTLMNV | L13 | VVNAANVYLK | L7; L6 | KSNIIRGW | L55; L54; L52; |
| | | | | | | L65; L53; L48 |
| 2995 | RVWTLMNVLTL | L16 | VVNIQKEI | L48; L55 | KSNLKPFER | L21; L15; L73; |
| | | | | | | L55; L17; L7 |
| 2996 | RVYANLGERV | L24 | VVQLTSQWL | L60; L74 | KSYELQTP | L55; L54; L53; |
| | | | | | | L43; L46; L48 |
| 2997 | RWFLNRFT | L55 | VVRSIFSRT | L13; L43 | KTDGTLMI | L18; L68; L63; L1; |
| | | | | | | L54; L73 |
| 2998 | RWFLNRFTT | L16 | VVSDIDYVPL | L20; L67 | KVGGNYNYL | L65; L74; L16; |
| | | | | | | L73; L67; L58 |
| 2999 | RYFRLTLGVYDY | L14 | VVSDIDYVPLK | L6; L7 | LAAIMQLF | L59; L47; L52; |
| | | | | | | L49; L74; L58 |
| 3000 | RYLALYNK | L15 | VVSKVVKVTI | L20; L53 | LACFVLAAV | L50; L59; L47; |
| | | | | | | L20; L67; L70 |
| 3001 | RYLALYNKYK | L13 | VVTCLAYYF | L12; L52 | LAFLLFLV | L47; L50; L70; |
| | | | | | | L69; L59; L64 |
| 3002 | RYMNSQGLLP | L8 | VVYRGTTTYK | L6; L7 | LAKALRKV | L70; L47; L58; |
| | | | | | | L64; L23; L69 |
| 3003 | RYRIGNYK | L13 | VWTLMNVL | L62; L25 | LAKHCLHVV | L70; L47; L69; |
| | | | | | | L58; L50; L32 |
| 3004 | RYVDNNFCG | L8 | VYANLGERVR | L15; L17 | LALLLLDRL | L67; L47; L31; |
| | | | | | | L74; L50; L60 |
| 3005 | RYWEPEFYEAM | L14 | VYAWNRKRI | L9; L8 | LAMDEFIERY | L31; L49; L12; |
| | Y | | | | | L52; L18; L14 |
| 3006 | SAAEASKK | L19 | VYDPLQPELD | L62; L9 | LARGIVFM | L58; L70; L59; |
| | | | | | | L74; L47; L69 |
| 3007 | SAAEASKKPR | L19 | VYDYLVSTQ | L62; L9 | LAWPLIVT | L47; L59; L69; |
| | | | | | | L50; L31; L70 |
| 3008 | SAEVAVKMFDA | L18 | VYFASTEK | L71; L72 | LEIPRRNVA | L46; L38; L42; |
| | Y | | | | | L25; L37; L29 |
| 3009 | SAFAMMFVKHK | L7 | VYIGDPAQLP | L9; L8 | LENVAFNVV | L45; L29; L46; |
| | | | | | | L38; L42; L37 |
| 3010 | SAFVETVKG | L49 | VYIKNADI | L71; L8 | LFDESGEF | L62; L72; L68; |
| | | | | | | L63; L71; L1 |
| 3011 | SAGFPFNK | L7 | VYIKNADIV | L71; L72 | LFLMSFTVL | L72; L71; L8; L9; |
| | | | | | | L61; L62 |
| 3012 | SAGFSLWVYK | L7 | VYKQFDTYNL | L8; L9 | LFLTWICLL | L8; L9; L62; L72; |
| | | | | | | L71; L4 |
| 3013 | SAKPPPGD | L43 | VYLAVFDKNL | L9; L8 | LGAENSVAY | L26; L43; L27; |
| | | | | | | L12; L31; L59 |
| 3014 | SAKPPPGDQ | L43 | VYLPYPDPSR | L9; L15 | LHDELTGHML | L34; L35; L28; |
| | | | | | | L68; L63; L5 |
| 3015 | SALGKLQD | L65 | VYLPYPDPSRI | L9; L8 | LIMLIIFWF | L12; L26; L9; L52; |
| | | | | | | L60; L58 |
| 3016 | SALLAGTI | L47 | VYMPASWVMRI | L9; L8 | LIYSTAAL | L59; L61; L60; |
| | | | | | | L67; L43; L57 |
| 3017 | SALLEDEF | L59 | VYPVASPN | L72; L71 | LLAPLLSA | L3; L51; L50; L43; |
| | | | | | | L2; L23 |
| 3018 | SALVYDNKLK | L7 | VYRGTTTYK | L13; L6 | LLDRLNQL | L68; L1; L63; L5; |
| | | | | | | L26; L74 |
| 3019 | SAPTLVPQ | L57 | VYRGTTTYKL | L8; L9 | LLIGLAKRF | L12; L26; L11; |
| | | | | | | L43; L49; L9 |
| 3020 | SAPTLVPQEH | L57 | VYSDVENPHLM | L9; L8 | LLWPVTLAC | L6; L5; L4; L2; |
| | | | | | | L12; L43 |
| 3021 | SAQCFKMFYK | L7 | VYSRVKNLN | L9; L8 | LMWLIINLV | L47; L48; L5; L64; |
| | | | | | | L50; L4 |
| 3022 | SARIVYTA | L50 | VYSSANNCTF | L9; L8 | LNDNLLEIL | L68; L67; L62; |
| | | | | | | L63; L5; L34 |
| 3023 | SARIVYTAC | L43 | VYSTGSNV | L71; L72 | LPAADLDDF | L31; L49; L56; |
| | | | | | | L32; L22; L39 |
| 3024 | SASIVAGGI | L49 | VYYHKNNKS | L8; L9 | LPINVIVED | L50; L31; L32; |
| | | | | | | L49; L56; L47 |
| 3025 | SASKIITLKKR | L19 | VYYPDKVFRS | L8; L9 | LPQGFSAL | L22; L56; L39; |
| | | | | | | L47; L32; L31 |
| 3026 | SASTSAFV | L58 | VYYSQLMC | L71; L72 | LPQNAVVKI | L47; L50; L49; |
| | | | | | | L39; L32; L31 |
| 3027 | SAVLQSGF | L58 | VYYTSNPT | L71; L72 | LPRVFSAV | L47; L51; L50; |
| | | | | | | L22; L39; L23 |
| 3028 | SAVLQSGFR | L19 | WEIQQVVD | L37; L29 | LPSLATVAY | L31; L50; L49; |
| | | | | | | L22; L56; L51 |
| 3029 | SAYENFNQHEV | L67 | WESGVKDCV | L45; L42 | LPTEVLTEE | L31; L56; L50; |
| | | | | | | L32; L49; L51 |
| 3030 | SAYENFNQHEVL | L67 | WFFSNYLKRR | L17; L15 | LQSADAQSF | L26; L31; L27; |
| | | | | | | L34; L28; L58 |
| 3031 | SCGNFKVTK | L7 | WFLAYILFTR | L17; L15 | LRDGWEIVKF | L30; L34; L65; |
| | | | | | | L49; L63; L35 |
| 3032 | SCKRVLNV | L48 | WFVTQRNF | L71; L72 | LRVESSSKL | L30; L25; L66; |
| | | | | | | L35; L64; L28 |
| 3033 | SCLPFTINC | L7 | WIFGTTLDSK | L6; L7 | LSDRVVFVLW | L52; L53; L1; L54; |
| | | | | | | L18; L49 |
| 3034 | SDADSTLI | L33 | WLDDVVYCP | L5; L2 | LSDTLKNL | L68; L63; L1; L60; |
| | | | | | | L62; L74 |
| 3035 | SDDYIATN | L33 | WLKQLIKV | L3; L23 | LSLREVRTI | L55; L48; L53; |
| | | | | | | L47; L52; L65 |
| 3036 | SDEFSSNV | L33 | WLLLTILTSL | L4; L2 | LTLQQIEL | L55; L60; L53; |
| | | | | | | L59; L67; L61 |
| 3037 | SDEFSSNVA | L38 | WPLIVTALR | L21; L17 | LTLVYKVYY | L52; L1; L12; L18; |
| | | | | | | L14; L65 |
| 3038 | SDEVARDL | L33 | WPVTLACFVL | L32; L56 | LTQDHVDIL | L67; L69; L73; |
| | | | | | | L60; L61; L70 |
| 3039 | SDFVRATATI | L33 | WQLALSKGV | L24; L48 | LTWICLLQF | L53; L52; L59; |
| | | | | | | L16; L1; L18 |
| 3040 | SDGTGTIY | L65 | WRKAVFISPY | L66; L30 | LVFLFVAAI | L47; L20; L3; L50; |
| | | | | | | L59; L64 |
| 3041 | SDIEVTGD | L33 | WSFNPETNI | L24; L53 | LVGLMWLSY | L12; L1; L18; L43; |
| | | | | | | L6; L59 |
| 3042 | SDKFTDGV | L33 | WSLFFFLY | L1; L18 | LVIGFLFLTW | L10; L52; L12; |
| | | | | | | L16; L49; L6 |
| 3043 | SDLATNNL | L33 | WSMATYYL | L74; L55 | LVLIMLIIF | L52; L31; L59; |
| | | | | | | L12; L74; L53 |
| 3044 | SDLATNNLV | L33 | WSYSGQST | L43; L55 | LVLSVNPYV | L4; L5; L2; L47; |
| | | | | | | L13; L20 |
| 3045 | SDRDLYDKLQF | L65 | WTIEYPIIGD | L11; L10 | LVYCFLGY | L18; L59; L12; L1; |
| | | | | | | L26; L6 |
| 3046 | SDRVVFVLW | L52 | WTNAGDYIL | L32; L55 | LVYCFLGYF | L11; L58; L59; |
| | | | | | | L12; L10; L52 |
| 3047 | SDSPCESH | L33 | YAISAKNR | L17; L19 | LVYFLQSI | L47; L59; L24; |
| | | | | | | L48; L55; L25 |
| 3048 | SDVENPHL | L33 | YAISAKNRA | L50; L46 | LWPVTLACF | L62; L9; L57; L71; |
| | | | | | | L8; L72 |
| 3049 | SDVETKDV | L33 | YAISAKNRAR | L17; L19 | LYALVYFL | L62; L66; L72; |
| | | | | | | L71; L9; L64 |
| 3050 | SDVETKDVV | L37 | YASALWEIQ | L67; L49 | LYENAFLPF | L9; L8; L72; L71; |
| | | | | | | L62; L12 |
| 3051 | SDVKCTSV | L33 | YASQGLVA | L50; L61 | LYIDINGNL | L8; L9; L72; L71; |
| | | | | | | L62; L66 |
| 3052 | SDVKCTSVV | L33 | YCIDGALLTK | L7; L6 | LYIIKLIF | L71; L72; L9; L8; |
| | | | | | | L56; L23 |
| 3053 | SDVLLPLTQY | L29 | YDFAVSKGF | L33; L27 | LYSPIFLI | L9; L47; L8; L64; |
| | | | | | | L66; L62 |
| 3054 | SEAFLIGCN | L40 | YDLSVVNAR | L17; L19 | LYYQNNVF | L9; L72; L71; L8; |
| | | | | | | L26; L66 |
| 3055 | SEAGVCVS | L29 | YDYLVSTQ | L33; L48 | MATNYDLSV | L70; L69; L50; |
| | | | | | | L32; L73; L47 |
| 3056 | SEAKCWTETDL | L36 | YECLYRNRDV | L45; L38 | MAYITGGVV | L47; L50; L70; |
| | | | | | | L69; L61; L49 |
| 3057 | SECVLGQSK | L40 | YEDFLEYHDV | L38; L45 | MCDIRQLLF | L1; L18; L33; L49; |
| | | | | | | L62; L34 |
| 3058 | SEDAQGMDNLA | L38 | YEKLKPVLDW | L41; L40 | MEIDFLELAM | L46; L40; L41; |
| | | | | | | L36; L29; L42 |
| 3059 | SEDKRAKVTS | L42 | YELQTPFEIKL | L36; L41 | MFLARGIVF | L71; L72; L9; L8; |
| | | | | | | L12; L31 |
| 3060 | SEFDRDAAMQ | L40 | YEPLTQDHV | L45; L36 | MKIILFLAL | L67; L27; L61; |
| | | | | | | L44; L73; L59 |
| 3061 | SEFSSLPSYA | L46 | YERHSLSHF | L29; L58 | MSAPPAQYEL | L20; L67; L60; |
| | | | | | | L61; L53; L54 |
| 3062 | SEHDYQIG | L29 | YESLRPDTR | L33; L29 | MSYEDQDAL | L67; L55; L61; |
| | | | | | | L53; L60; L59 |
| 3063 | SEIIGYKAID | L42 | YFCTCYFGLF | L9; L62 | MYIFFASF | L72; L9; L71; L62; |
| | | | | | | L8; L59 |
| 3064 | SEKQVEQK | L29 | YFDKAGQKTY | L1; L12 | NAFLPFAM | L70; L59; L67; |
| | | | | | | L31; L50; L47 |
| 3065 | SEKSYELQT | L42 | YFGLFCLL | L62; L67 | NCDTLKEIL | L68; L34; L63; |
| | | | | | | L67; L62; L35 |
| 3066 | SEMVMCGGSL | L36 | YFIKGLNN | L71; L72 | NEEIAIILA | L42; L38; L46; |
| | | | | | | L37; L40; L29 |
| 3067 | SEPVLKGVKLHY | L41 | YFITDAQTG | L71; L72 | NEFACVVA | L46; L29; L25; |
| | | | | | | L37; L42; L38 |
| 3068 | SEVGPEHS | L29 | YFNKKDWY | L71; L72 | NEKTHVQL | L23; L25; L37; |
| | | | | | | L29; L33; L45 |
| 3069 | SEVVLKKLKK | L41 | YFPLQSYG | L62; L71 | NFKDQVIL | L62; L71; L72; |
| | | | | | | L67; L66; L23 |
| 3070 | SEYDYVIFTQTT | L41 | YFTSDYYQLY | L12; L14 | NFNKDFYDF | L8; L9; L49; L12; |
| | | | | | | L62; L72 |
| 3071 | SEYKGPIT | L29 | YFVLTSHTVM | L71; L72 | NFYEPQII | L48; L62; L25; |
| | | | | | | L71; L72; L47 |
| 3072 | SEYTGNYQ | L45 | YFYTSKTT | L71; L72 | NGDSEVVL | L63; L68; L62; |
| | | | | | | L67; L35; L28 |
| 3073 | SFDLGDELG | L62 | YGRSGETL | L23; L59 | NIDYDCVSF | L68; L63; L62; |
| | | | | | | L49; L34; L5 |
| 3074 | SFDNFKFV | L62 | YHKNNKSWM | L28; L58 | NKGEDIQLL | L64; L34; L44; |
| | | | | | | L66; L35; L27 |
| 3075 | SFDNFKFVC | L62 | YHNESGLKTIL | L28; L34 | NLDSCKRVL | L23; L63; L68; L5; |
| | | | | | | L62; L32 |
| 3076 | SFDVLKSE | L62 | YHYQECVRG | L34; L28 | NLDYIINL | L74; L62; L68; L5; |
| | | | | | | L63; L1 |
| 3077 | SFDVLKSED | L62 | YIDIGNYT | L1; L68 | NLYDKLVSS | L23; L3; L43; L4; |
| | | | | | | L2; L5 |
| 3078 | SFGPLVRKIF | L9 | YIICISTKH | L11; L43 | NMMVTNNTF | L34; L9; L8; L71; |
| | | | | | | L72; L26 |
| 3079 | SFKEELDKYF | L9 | YIKWPWYIW | L43; L16 | NNELSPVAL | L68; L35; L67; |
| | | | | | | L62; L28; L25 |
| 3080 | SFLGRYMSA | L71 | YILFTRFF | L60; L59 | NPTIQKDVL | L23; L39; L32; |
| | | | | | | L31; L56; L22 |
| 3081 | SFLPGVYSVIY | L12 | YIWLGFIAG | L67; L59 | NRQFHQKLL | L64; L66; L35; |
| | | | | | | L30; L34; L25 |
| 3082 | SFVSEETGTLI | L8 | YKGPITDVFY | L27; L12 | NRYFRLTL | L25; L35; L66; |
| | | | | | | L65; L64; L28 |
| 3083 | SFYVYSRVKNL | L71 | YKHWPQIAQ | L27; L43 | NSASFSTF | L70; L54; L55; |
| | | | | | | L59; L58; L53 |
| 3084 | SGARSKQRR | L19 | YKLDGVVC | L27; L44 | NSHEGKTFY | L43; L1; L14; L18; |
| | | | | | | L58; L26 |
| 3085 | SGETLGVLV | L63 | YKLDGVVCT | L44; L27 | NVAFNVVNK | L19; L7; L6; L13; |
| | | | | | | L17; L20 |
| 3086 | SGFAAYSR | L15 | YKSVNITF | L27; L66 | NVYADSFVIR | L19; L17; L7; L21; |
| | | | | | | L15; L6 |
| 3087 | SGFAAYSRYR | L15 | YLGGMSYY | L1; L43 | NYYKKDNSY | L71; L72; L66; |
| | | | | | | L14; L12; L8 |
| 3088 | SGFRKMAF | L29 | YLKHGGGVA | L51; L3 | PANSIVCRF | L58; L49; L54; |
| | | | | | | L43; L70; L69 |
| 3089 | SGINASVV | L48 | YLKRRVVFN | L3; L23 | PVYSFLPGV | L3; L20; L4; L11; |
| | | | | | | L5; L2 |
| 3090 | SGNLLLDKR | L15 | YLPQNAVV | L57; L5 | PYNMRVIHF | L9; L8; L72; L71; |
| | | | | | | L66; L62 |
| 3091 | SGVTFQSAV | L48 | YLPQNAVVK | L57; L6 | QALLKTVQF | L33; L52; L31; |
| | | | | | | L53; L27; L59 |
| 3092 | SGVTFQSAVKR | L19 | YLPYPDPSR | L5; L57 | QEGVLTAVV | L42; L45; L38; |
| | | | | | | L37; L36; L40 |
| 3093 | SGVVTTVMF | L49 | YLPYPDPSRI | L5; L57 | QELYSPIFL | L36; L41; L40; |
| | | | | | | L37; L29; L45 |
| 3094 | SGWTAGAAA | L51 | YLQPRTFLLK | L6; L7 | QESFGGASC | L40; L42; L38; |
| | | | | | | L36; L41; L45 |
| 3095 | SGWTAGAAAY | L14 | YLYFIKGLNNL | L2; L4 | QEVFAQVKQ | L40; L41; L36; |
| | | | | | | L38; L37; L46 |
| 3096 | SGWTAGAAAYY | L14 | YMLTYNKV | L48; L47 | QLIKVTLVFL | L2; L4; L3; L5; |
| | | | | | | L74; L20 |
| 3097 | SHEGKTFY | L1 | YMPYFFTLLL | L57; L5 | QPGQTFSVL | L22; L39; L56; |
| | | | | | | L32; L65; L31 |
| 3098 | SHFAIGLA | L28 | YNVTQAFGR | L19; L17 | QPYRVVVLSF | L31; L22; L49; |
| | | | | | | L47; L39; L32 |
| 3099 | SHGKQVVSD | L35 | YNYEPLTQD | L46; L43 | QQGEVPVSI | L24; L44; L34; |
| | | | | | | L48; L35; L28 |
| 3100 | SHKPPISFP | L28 | YPANSIVCRF | L49; L31 | QSINFVRII | L55; L70; L69; |
| | | | | | | L64; L48; L73 |
| 3101 | SHKPPISFPL | L28 | YPDKVFRSSVL | L32; L22 | QSTQLGIEF | L53; L55; L52; |
| | | | | | | L54; L58; L26 |
| 3102 | SHLLLVAAGL | L40 | YPGQGLNGY | L49; L31 | QYGRSGETL | L9; L72; L8; L71; |
| | | | | | | L66; L34 |
| 3103 | SHNIALIWNV | L28 | YPIIGDELK | L49; L32 | QYLNTLTL | L72; L71; L9; L8; |
| | | | | | | L66; L48 |
| 3104 | SHQSDIEVTG | L34 | YPKCDRAM | L39; L32 | RAFGEYSHV | L58; L69; L24; |
| | | | | | | L70; L13; L73 |
| 3105 | SHSQLGGLHLLI | L34 | YPLECIKD | L50; L47 | RAGKASCTL | L53; L54; L44; |
| | | | | | | L56; L58; L61 |
| 3106 | SHTVMPLSAPTL | L28 | YPNASFDNFKF | L31; L49 | RDLICAQKF | L33; L55; L53; L8; |
| | | | | | | L37; L54 |
| 3107 | SHVVAFNTLLF | L34 | YPPDEDEEE | L49; L32 | RDLYDKLQF | L33; L65; L53; |
| | | | | | | L16; L8; L52 |
| 3108 | SIAATRGATV | L3 | YPQVNGLTS | L50; L49 | REAVGTNLP | L38; L42; L46; |
| | | | | | | L45; L36; L37 |
| 3109 | SIDAFKLNIKL | L68 | YPSARIVY | L31; L22 | REEAIRHV | L45; L37; L33; |
| | | | | | | L38; L48; L42 |
| 3110 | SIFSRTLETA | L3 | YPSARIVYTA | L50; L51 | REETGLLMP | L38; L42; L46; |
| | | | | | | L36; L37; L45 |
| 3111 | SIIKTIQPRV | L48 | YPTLNISDEF | L31; L49 | REFLTRNP | L33; L46; L38; |
| | | | | | | L37; L45; L48 |
| 3112 | SIISNEKQEI | L24 | YQHEETIYN | L26; L24 | REHEHEIA | L38; L46; L37; |
| | | | | | | L42; L29; L33 |
| 3113 | SIKWADNNCY | L14 | YQLRARSV | L48; L25 | RENNRVVI | L37; L45; L36; |
| | | | | | | L33; L48; L29 |
| 3114 | SINFVRII | L48 | YQTSNFRV | L24; L48 | RFFYVLGL | L71; L72; L65; |
| | | | | | | L33; L48; L62 |
| 3115 | SINFVRIIM | L39 | YRAFDIYND | L65; L30 | RFLYIIKL | L8; L33; L72; L71; |
| | | | | | | L62; L48 |
| 3116 | SIPCSVCL | L57 | YRARAGEAA | L35; L46 | RFPNITNL | L72; L62; L71; |
| | | | | | | L57; L8; L9 |
| 3117 | SIVRFPNI | L48 | YRIGNYKLN | L30; L64 | RIAGHHLGR | L21; L15; L6; L19; |
| | | | | | | L7; L17 |
| 3118 | SIWNLDYII | L24 | YRLYLDAYNM | L30; L66 | RISNCVADY | L14; L18; L26; |
| | | | | | | L16; L6; L12 |
| 3119 | SKFPLKLR | L15 | YRRATRRI | L48; L64 | RLQSLQTYV | L2; L4; L24; L5; |
| | | | | | | L3; L13 |
| 3120 | SKFPLKLRG | L44 | YRSLPGVF | L66; L35 | RLSFKELLV | L24; L73; L2; L18; |
| | | | | | | L4; L3 |
| 3121 | SKFYGGWHN | L27 | YRSLPGVFC | L35; L30 | RLSFKELLVY | L18; L6; L12; L14; |
| | | | | | | L16; L26 |
| 3122 | SKFYGGWHNM | L27 | YSDVENPH | L1; L68 | RLTLGVYDY | L14; L26; L18; |
| | | | | | | L16; L12; L27 |
| 3123 | SKFYGGWHNML | L44 | YSDVENPHLMG | L1; L18 | RMYIFFASFY | L14; L27; L18; |
| | | | | | | L26; L6; L12 |
| 3124 | SKGRLIIRE | L44 | YSFLPGVYSV | L50; L20 | RNAGIVGVL | L44; L65; L73; |
| | | | | | | L56; L39; L69 |
| 3125 | SKHTPINL | L66 | YSGVVTTVMF | L53; L43 | RPDTRYVL | L39; L22; L56; |
| | | | | | | L32; L65; L63 |
| 3126 | SKILGLPTQ | L27 | YSHLLLVA | L50; L59 | RPQIGVVREF | L39; L56; L22; |
| | | | | | | L49; L31; L65 |
| 3127 | SKLIEYTDF | L27 | YSSANNCTFEY | L1; L18 | RSAAKKNNL | L53; L73; L54; |
| | | | | | | L44; L56; L16 |
| 3128 | SKLINIIIWF | L27 | YSTAALGV | L73; L48 | RSEKSYEL | L55; L54; L63; |
| | | | | | | L65; L18; L53 |
| 3129 | SKLINIIIWFL | L44 | YSTGSNVFQTR | L17; L19 | RSVSPKLFI | L73; L55; L53; |
| | | | | | | L54; L24; L65 |
| 3130 | SKLWAQCVQL | L44 | YSVLYNSASF | L43; L60 | RTATKAYNV | L73; L13; L18; |
| | | | | | | L20; L53; L21 |
| 3131 | SKQRRPQGL | L44 | YSYATHSD | L48; L43 | RVKNLNSSR | L15; L21; L13; |
| | | | | | | L17; L16; L14 |
| 3132 | SKRSFIEDLL | L44 | YSYATHSDKF | L54; L58 | RVLSNLNL | L54; L74; L56; |
| | | | | | | L16; L73; L55 |
| 3133 | SKRVDFCGK | L13 | YTACSHAAV | L20; L70 | RVQPTESIVRF | L54; L16; L52; |
| | | | | | | L53; L26; L14 |
| 3134 | SKSPIQYI | L64 | YTFEKGDY | L1; L14 | RVVISSDVL | L53; L54; L65; |
| | | | | | | L56; L26; L44 |
| 3135 | SKSPIQYID | L44 | YTKVDGVDV | L58; L70 | RYMNSQGLL | L8; L9; L72; L71; |
| | | | | | | L14; L62 |
| 3136 | SKTPEEHF | L27 | YTMADLVYAL | L20; L67 | SAEVAVKMF | L63; L58; L49; |
| | | | | | | L68; L70; L31 |
| 3137 | SKVGGNYNYL | L44 | YTNSFTRGVYY | L1; L12 | SAIGKIQDSL | L67; L61; L32; |
| | | | | | | L10; L58; L60 |
| 3138 | SKVVKVTIDY | L27 | YTPHTVLQAV | L57; L5 | SAKPPPGDQF | L43; L58; L26; |
| | | | | | | L52; L11; L53 |
| 3139 | SLAATVRLQA | L3 | YTQLCQYL | L60; L70 | SALGKLQDV | L58; L24; L69; |
| | | | | | | L70; L4; L64 |
| 3140 | SLAGSYKDW | L10 | YTVEEAKTVLK | L7; L19 | SALNHTKKW | L52; L69; L49; |
| | | | | | | L54; L10; L70 |
| 3141 | SLATVAYF | L74 | YTVSCLPF | L59; L60 | SANLAATKM | L58; L63; L31; |
| | | | | | | L68; L32; L70 |
| 3142 | SLDNVLST | L68 | YVDNNFCGPDG | L1; L18 | SDIDITFL | L74; L37; L33; |
| | | | Y | | | L65; L29; L25 |
| 3143 | SLEIPRRNV | L13 | YVDNSSLT | L1; L63 | SEETGTLIV | L45; L38; L42; |
| | | | | | | L37; L36; L46 |
| 3144 | SLENVAFNVV | L5 | YVDTPNNT | L68; L1 | SEIIGYKA | L38; L46; L42; |
| | | | | | | L37; L29; L45 |
| 3145 | SLKEGQIN | L43 | YVFIKRSD | L65; L23 | SEYCRHGTC | L25; L33; L46; |
| | | | | | | L45; L37; L38 |
| 3146 | SLKVPATVS | L43 | YVFTGYRV | L20; L48 | SEYKGPITDV | L45; L37; L36; |
| | | | | | | L42; L46; L24 |
| 3147 | SLLLCRMNSR | L21 | YVFTGYRVT | L10; L69 | SFDLGDEL | L62; L68; L67; |
| | | | | | | L71; L63; L72 |
| 3148 | SLLMPILTLT | L6 | YVGYLQPRTF | L65; L55 | SFIEDLLF | L8; L9; L62; L72; |
| | | | | | | L71; L12 |
| 3149 | SLLMPILTLTR | L21 | YVKPGGTSSG | L43; L10 | SFKEELDKY | L12; L14; L13; |
| | | | | | | L11; L10; L26 |
| 3150 | SLLSKGRL | L74 | YVLGLAAI | L59; L67 | SFKELLVYA | L13; L3; L51; L42; |
| | | | | | | L17; L15 |
| 3151 | SLLSKGRLII | L16 | YVRNLQHR | L17; L15 | SFSASTSAF | L72; L71; L9; L62; |
| | | | | | | L12; L8 |
| 3152 | SLNGVTLIG | L6 | YVTQQLIRA | L50; L46 | SGLDSLDTY | L14; L27; L12; |
| | | | | | | L26; L16; L52 |
| 3153 | SLPFGWLI | L57 | YWVPRASAN | L71; L61 | SIIKTIQPR | L19; L21; L15; L7; |
| | | | | | | L17; L11 |
| 3154 | SLPFGWLIVGV | L5 | YWVPRASANI | L9; L8 | SIINNTVY | L26; L43; L58; |
| | | | | | | L29; L59; L14 |
| 3155 | SLPSYAAFAT | L57 | YYHKNNKSWM | L71; L72 | SIKNFKSVLY | L12; L43; L14; |
| | | | | | | L13; L58; L26 |
| 3156 | SLPVLQVR | L57 | YYKLGASQ | L71; L72 | SINFVRIIMR | L21; L19; L15; L7; |
| | | | | | | L17; L6 |
| 3157 | SLPVLQVRD | L5 | YYPDKVFRSSV | L71; L72 | SLAATVRL | L74; L56; L4; L2; |
| | | | | | | L44; L3 |
| 3158 | SLQTYVTQQ | L4 | YYPDKVFRSSVL | L71; L72 | SLFDMSKF | L26; L11; L27; |
| | | | | | | L16; L10; L43 |
| 3159 | SLQTYVTQQL | L4 | YYQLYSTQ | L71; L72 | SLNVAKSEF | L43; L26; L62; |
| | | | | | | L16; L57; L72 |
| 3160 | SLRCGACIRR | L21 | YYSDSPCESH | L72; L71 | SLPFGWLIV | L57; L24; L5; L3; |
| | | | | | | L2; L64 |
| 3161 | SLRVCVDTVR | L21 | YYTSNPTTFHL | L8; L9 | SLREVRTIKV | L3; L2; L13; L5; |
| | | | | | | L4; L24 |
| 3162 | SLSEQLRKQI | L3 | YYVWKSYV | L71; L72 | SLSHRFYRL | L4; L23; L5; L2; |
| | | | | | | L74; L3 |
| 3163 | SLSGFKLK | L6 | AAGLEAPFLY | L12; L18; | SLVKPSFYVY | L26; L12; L27; |
| | | | | L14 | | L14; L16; L10 |
| 3164 | SLSGFKLKD | L24 | AAVINGDRW | L54; L52; | SPRRARSV | L48; L51; L39; |
| | | | | L49 | | L22; L23; L47 |
| 3165 | SLSSTASALGK | L6 | AAVYRINWI | L69; L70; | SPRWYFYYL | L39; L22; L32; |
| | | | | L64 | | L56; L65; L13 |
| 3166 | SLVVRCSF | L43 | ACFVLAAVYR | L15; L21; | SPVALRQM | L56; L39; L22; |
| | | | | L7 | | L32; L51; L31 |
| 3167 | SLWVYKQFDTY | L18 | ACPLIAAV | L57; L33; | SQDLSVVSK | L7; L68; L35; L27; |
| | | | | L48 | | L28; L63 |
| 3168 | SMATNYDLSV | L3 | ACWHHSIGF | L33; L27; | SRTLSYYKL | L35; L30; L25; |
| | | | | L26 | | L66; L34; L64 |
| 3169 | SMDNSPNLA | L62 | ADKYVRNL | L33; L65; | SRYWEPEFY | L30; L66; L65; |
| | | | | L37 | | L64; L12; L14 |
| 3170 | SMDNSPNLAW | L68 | ADSNGTITV | L33; L37; | SSPDDQIGYY | L11; L14; L18; |
| | | | | L24 | | L43; L1; L10 |
| 3171 | SMKYFVKI | L48 | AEETRKLMP | L38; L42; | STASALGKL | L10; L20; L11; |
| | | | | L46 | | L58; L53; L73 |
| 3172 | SMMGFKMNYQV | L2 | AEGSRGGSQA | L46; L42; | STFEEAAL | L20; L67; L59; |
| | | | | L38 | | L28; L61; L60 |
| 3173 | SMQGAVDINK | L7 | AEILLIIMRT | L41; L40; | STQLGIEF | L59; L72; L60; |
| | | | | L45 | | L71; L53; L26 |
| 3174 | SNGTHWFVTQR | L19 | AEIRASANLAA | L38; L42; | STQWSLFFF | L53; L58; L7; L52; |
| | | | | L46 | | L12; L9 |
| 3175 | SNGTITVEEL | L65 | AEIVDTVSALV | L38; L40; | SVLLFLAF | L59; L33; L23; |
| | | | | L36 | | L29; L12; L60 |
| 3176 | SNHDLYCQV | L13 | AELEGIQYGR | L40; L15; | SVLYNSASF | L12; L16; L10; |
| | | | | L41 | | L26; L11; L52 |
| 3177 | SNLKPFER | L17 | AENVTGLF | L33; L29; | SYATHSDKF | L9; L8; L71; L72; |
| | | | | L40 | | L66; L12 |
| 3178 | SNNLDSKV | L48 | AENVTGLFKD | L42; L40; | SYFTEQPIDL | L72; L71; L9; L65; |
| | | | | L41 | | L66; L8 |
| 3179 | SNPTTFHL | L57 | AEVAVKMFDA | L42; L46; | SYFTSDYY | L71; L72; L14; |
| | | | | L38 | | L12; L65; L66 |
| 3180 | SNSGSDVL | L65 | AFGEYSHV | L62; L72; | SYFTSDYYQL | L8; L9; L71; L72; |
| | | | | L71 | | L66; L65 |
| 3181 | SNVANYQKV | L73 | AFGEYSHVV | L72; L71; | SYGADLKSF | L9; L72; L8; L71; |
| | | | | L42 | | L66; L10 |
| 3182 | SNYLKRRVVF | L27 | AFGEYSHVVAF | L71; L72; | TAPHGHVM | L57; L63; L70; |
| | | | | L9 | | L32; L59; L66 |
| 3183 | SNYSGVVTTV | L3 | AFGLVAEW | L71; L72; | TAVLRQWL | L70; L60; L67; |
| | | | | L62 | | L59; L74; L61 |
| 3184 | SPCESHGKQVV | L39 | AFHQECSL | L71; L72; | TEAFEKMVSL | L36; L37; L40; |
| | | | | L62 | | L41; L38; L45 |
| 3185 | SPFELEDFIP | L32 | AFLCLFLL | L8; L71; | TESKPSVEQ | L38; L36; L45; |
| | | | | L72 | | L46; L37; L42 |
| 3186 | SPFELEDFIPM | L31 | AFLCLFLLP | L12; L42; | TEVLTEEVVL | L36; L37; L40; |
| | | | | L8 | | L41; L44; L65 |
| 3187 | SPFVMMSAPPA | L50 | AFNVVNKGHF | L9; L62; | TEVPVAIHAD | L40; L37; L38; |
| | | | | 7L2 | | L41; L29; L36 |
| 3188 | SPISEHDY | L49 | AFQTVKPGNF | L72; L9; | TFDNLKTLL | L62; L68; L63; L8; |
| | | | | 7L1 | | L34; L9 |
| 3189 | SPNECNQM | L39 | AFVNLKQLPF | L12; L71; | TFEYVSQPF | L62; L72; L9; L8; |
| | | | | L72 | | L71; L12 |
| 3190 | SPNFSKLINII | L39 | AFVVFLLV | L48; L62; | TIAFGGCVF | L43; L26; L10; |
| | | | | L73 | | L58; L28; L11 |
| 3191 | SPRRARSVAS | L22 | AFVVFLLVTL | L71; L72; | TIYSLLLCR | L17; L6; L19; L15; |
| | | | | L8 | | L7; L21 |
| 3192 | SPTKLNDLCF | L49 | AGFSLWVYK | L7; L6; | TLAILTAL | L23; L3; L25; L59; |
| | | | | 3L1 | | L4; L74 |
| 3193 | SPYNSQNA | L51 | AGLPYGANK | L7; L13; | TLIGDCATV | L3; L2; L4; L24; |
| | | | | 6L | | L5; L20 |
| 3194 | SQAWQPGV | L48 | AHFPREGV | L28; L33; | TLIVNSVLLF | L12; L9; L6; L26; |
| | | | | L25 | | L8; L16 |
| 3195 | SQAWQPGVAM | L28 | AHFPREGVFV | L28; L34; | TLKSFTVEK | L13; L43; L6; L21; |
| | | | | L35 | | L17; L7 |
| 3196 | SQDLSVVS | L68 | AHGFELTSM | L28; L34; | TLQAIASEF | L26; L72; L12; |
| | | | | L35 | | L16; L9; L14 |
| 3197 | SQGLVASI | L48 | AIDGGVTRD | L63; L18; | TLQCIMLVY | L26; L14; L12; |
| | | | | L68 | | L18; L6; L16 |
| 3198 | SQGSEYDYV | L24 | AIGLALYY | L18; L1; | TLVPQEHYV | L2; L5; L4; L3; |
| | | | | L12 | | L20; L64 |
| 3199 | SQHTMLVKQ | L24 | AIHADQLTP | L46; L42; | TPCSFGGVSV | L51; L50; L39; |
| | | | | L38 | | L22; L32; L56 |
| 3200 | SQLMCQPI | L48 | AIILASFSA | L38; L42; | TPKDHIGTR | L17; L51; L39; |
| | | | | L46 | | L49; L15; L19 |
| 3201 | SQLMCQPIL | L44 | AILTALRL | L74; L73; | TPKYKFVRI | L39; L23; L47; |
| | | | | L60 | | L49; L51; L32 |
| 3202 | SQLMCQPILL | L44 | AISAKNRAR | L21; L15; | TQDHVDIL | L63; L35; L68; |
| | | | | L7 | | L62; L28; L34 |
| 3203 | SQMEIDFL | L44 | AISMWALII | L73; L24; | TQWSLFFFL | L44; L24; L34; L4; |
| | | | | L69 | | L35; L8 |
| 3204 | SQNAVASKIL | L44 | AIVVTCLAYY | L14; L12; | TQYEYGTED | L27; L43; L24; |
| | | | | L18 | | L26; L48; L65 |
| 3205 | SQPFLMDL | L57 | AKPPPGDQF | L27; L66; | TRVLSNLNL | L35; L25; L34; |
| | | | | L9 | | L30; L64; L66 |
| 3206 | SQQTVGQQD | L24 | AKSHNIAL | L44; L66; | TSHKLVLSV | L73; L70; L24; |
| | | | | L35 | | L69; L48; L64 |
| 3207 | SQWLTNIFGTVY | L27 | AKYTQLCQY | L27; L14; | TSMKYFVKI | L73; L55; L48; |
| | | | | L26 | | L24; L53; L20 |
| 3208 | SQYSLRLID | L24 | ALAPNMMV | L2; L3; | TTAYANSVF | L53; L52; L10; |
| | | | | L4 | | L54; L70; L58 |
| 3209 | SRGGSQAS | L65 | ALAYYNTTK | L6; L7; | TTDPSFLGRY | L1; L18; L14; L10; |
| | | | | L21 | | L11; L12 |
| 3210 | SRGGSQASS | L65 | ALCTFLLNK | L6; L7; | TTITVNVL | L70; L55; L67; |
| | | | | L13 | | L28; L69; L35 |
| 3211 | SRGGSQASSR | L30 | ALDISASI | L68; L63; | TTIVNGVRR | L19; L17; L15; |
| | | | | L62 | | L21; L7; L11 |
| 3212 | SRIGMEVT | L35 | ALGVLMSNL | L4; L3; | TTLEETKFL | L53; L55; L73; L4; |
| | | | | L74 | | L10; L60 |
| 3213 | SRIIPARARV | L30 | ALILAYCNK | L13; L7; | TVLPPLLTD | L6; L7; L51; L16; |
| | | | | L6 | | L4; L12 |
| 3214 | SRIKASMPTTI | L34 | ALKYLPIDK | L13; L6; | TVREVLSDR | L15; L17; L19; |
| | | | | L7 | | L21; L11; L6 |
| 3215 | SRILGAGCFV | L30 | ALLADKFPVL | L2; L4; | TYASQGLVA | L46; L8; L51; L9; |
| | | | | L5 | | L72; L38 |
| 3216 | SRLDKVEAEV | L30 | ALLEDEFTP | L2; L4; | TYNLWNTF | L62; L9; L72; L8; |
| | | | | L16 | | L71; L33 |
| 3217 | SRLSFKELLV | L30 | ALLKTVQFC | L4; L24; | VADYSVLYN | L68; L67; L63; L1; |
| | | | | 2L | | L74; L18 |
| 3218 | SRLSFKELLVY | L30 | ALLTLQQIEL | L4; L2; | VAFELWAKR | L19; L17; L15; |
| | | | | L44 | | L21; L7; L69 |
| 3219 | SRNLQEFKP | L30 | ALNLGETF | L26; L27; | VAGDSGFAAY | L59; L14; L12; |
| | | | | L23 | | L31; L43; L18 |
| 3220 | SRNLQEFKPR | L30 | ALNNIINNAR | L21; L15; | VAGGIVAI | L67; L70; L47; |
| | | | | L6 | | L68; L74; L69 |
| 3221 | SRNSSRNST | L30 | ALQDAYYRA | L2; L4; | VAIHADQL | L67; L74; L60; |
| | | | | L5 | | L61; L70; L59 |
| 3222 | SRNSTPGSS | L30 | ALVYDNKLK | L7; L6; | VDDPCPIHF | L62; L63; L74; |
| | | | | L13 | | L33; L68; L9 |
| 3223 | SRNSTPGSSR | L30 | AMQVESDDY | L14; L26; | VEAEVQIDRL | L36; L41; L40; |
| | | | | L18 | | L45; L37; L29 |
| 3224 | SRNYIAQVDV | L30 | AMRPNFTIK | L6; L13; | VEVEKGVL | L29; L37; L36; |
| | | | | L7 | | L45; L33; L25 |
| 3225 | SRQRLTKY | L66 | ANDPVGFTLK | L7; L6; | VEVEKGVLP | L38; L46; L42; |
| | | | | L18 | | L45; L36; L37 |
| 3226 | SRQRLTKYTM | L30 | ANNAAIVL | L73; L65; | VEYCPIFFI | L45; L37; L36; |
| | | | | L71 | | L47; L41; L40 |
| 3227 | SRVKNLNSS | L30 | APAHISTI | L47; L22; | VFAFPFTIY | L12; L72; L26; |
| | | | | L39 | | L71; L9; L31 |
| 3228 | SRVKNLNSSR | L30 | APHGVVFLHV | L51; L50; | VFITLCFTL | L8; L9; L72; L71; |
| | | | | L22 | | L62; L67 |
| 3229 | SRVLGLKT | L25 | APLVGTPV | L22; L51; | VFLLVTLAI | L71; L72; L8; L9; |
| | | | | L50 | | L84; L47 |
| 3230 | SRVLGLKTLA | L30 | APLVGTPVC | L22; L51; | VFLVLLPLV | L9; L47; L4; L48; |
| | | | | L31 | | L62; L8 |
| 3231 | SRYWEPEFYE | L30 | APTLVPQEH | L56; L22; | VFPPTSFGPL | L72; L9; L57; L71; |
| | | | | L39 | | L8; L62 |
| 3232 | SRYWEPEFYEA | L30 | AQALNTLVK | L7; L6; | VFVLWAHGF | L9; L8; L62; L72; |
| | | | | L27 | | L71; L12 |
| 3233 | SRYWEPEFYEAM | L30 | AQDGNAAI | L68; L63; | VGMQKYSTL | L23; L25; L57; |
| | | | | L35 | | L61; L8; L44 |
| 3234 | SSAINRPQI | L48 | AQKFNGLTVL | L44; L26; | VGTNLPLQL | L73; L69; L64; |
| | | | | L27 | | L44; L61; L67 |
| 3235 | SSDNIALLVQ | L18 | AQPCSDKAY | L26; L27; | VIGFLFLTW | L12; L16; L67; L9; |
| | | | | L14 | | L73; L52 |
| 3236 | SSDVLVNN | L68 | AQSFLNGF | L26; L33; | VLCNSQTSL | L57; L2; L5; L68; |
| | | | | L27 | | L3; L4 |
| 3237 | SSEAFLIGC | L18 | AQVLSEMVM | L28; L27; | VLPFNDGVY | L12; L26; L14; |
| | | | | L44 | | L57; L18; L1 |
| 3238 | SSEAFLIGCNY | L1 | ARDLICAQKF | L30; L34; | VLYYQNNVF | L26; L16; L27; L9; |
| | | | | L35 | | L8; L12 |
| 3239 | SSEIIGYK | L1 | ARKSAPLIEL | L66; L30; | VPANSTVLSF | L22; L31; L49; |
| | | | | L56 | | L39; L56; L9 |
| 3240 | SSEIIGYKA | L42 | ARLYYDSMSY | L30; L66; | VPGLPGTIL | L22; L39; L56; |
| | | | | L65 | | L31; L51; L47 |
| 3241 | SSGDATTA | L65 | ARSVSPKL | L66; L35; | VPLHGTIL | L56; L47; L22; |
| | | | | L28 | | L23; L39; L31 |
| 3242 | SSGVVNPV | L48 | ARTAPHGHVM | L30; L66; | VQAGNVQLR | L21; L15; L19; |
| | | | | L35 | | L24; L17; L7 |
| 3243 | SSGVVNPVM | L70 | ARTVAGVSI | L35; L30; | VQTIEVNSF | L26; L27; L9; L34; |
| | | | | L34 | | L8; L16 |
| 3244 | SSGWTAGAAAY | L14 | ARVECFDKF | L30; L66; | VRQCSGVTF | L66; L34; L30; |
| | | | | L34 | | L35; L9; L72 |
| 3245 | SSIVITSG | L43 | ASANLAATK | L7; L19; | VSEETGTL | L63; L68; L1; L60; |
| | | | | L13 | | L55; L62 |
| 3246 | SSIVITSGD | L43 | ASDTYACW | L54; L18; | VSEETGTLI | L63; L1; L18; L68; |
| | | | | L1 | | L55; L48 |
| 3247 | SSKCVCSVI | L70 | ASEFSSLPSY | L18; L1; | VSFLAHIQW | L52; L53; L54; |
| | | | | L14 | | L70; L69; L16 |
| 3248 | SSKTPEEH | L43 | ASFNYLKSP | L46; L69; | VSLLSVLL | L74; L55; L60; |
| | | | | L43 | | L53; L73; L48 |
| 3249 | SSNFGAISS | L43 | ASFRLFAR | L15; L21; | VSTGYHFREL | L60; L73; L55; |
| | | | | L7 | | L61; L59; L70 |
| 3250 | SSNVANYQK | L7 | ASFSASTSA | L46; L51; | VSTIQRKY | L55; L48; L52; |
| | | | | L43 | | L54; L14; L1 |
| 3251 | SSPDDQIGYYR | L15 | ASKIITLKK | L13; L7; | VTFFIYNKI | L20; L55; L53; |
| | | | | L6 | | L24; L70; L16 |
| 3252 | SSQAWQPGV | L48 | ASLKELLQN | L7; L73; | VTFGDDTVI | L53; L52; L70; |
| | | | L3 | 5 | | L20; L55; L16 |
| 3253 | SSQGSEYD | L65 | ASRELKVTFF | L52; L53; | VTHSKGLYR | L15; L21; L17; L7; |
| | | | | L43 | | L6; L19 |
| 3254 | SSRVPDLL | L74 | ATNVVIKV | L73; L48; | VTNNTFTL | L70; L60; L55; |
| | | | | L70 | | L54; L53; L73 |
| 3255 | SSSEAFLI | L55 | ATNVVIKVC | L73; L52; | VTNNTFTLK | L7; L6; L13; L21; |
| | | | | L70 | | L19; L15 |
| 3256 | SSSEAFLIG | L73 | ATTRQVVNV | L73; L20; | VTWFHAIHV | L73; L20; L13; |
| | | | | L13 | | L69; L70; L63 |
| 3257 | SSSGWTAGAAAY | L14 | AVDAAKAY | L18; L1; | VVIKVCEF | L58; L74; L43; |
| | | | | L14 | | L26; L59; L54 |
| 3258 | SSSKTPEEHF | L53 | AVFDKNLYDK | L7; L6; | VVIKVCEFQF | L8; L53; L12; L52; |
| | | | | L13 | | L26; L54 |
| 3259 | SSSRSRNSSR | L19 | AVFQSASK | L7; L13; | VVTTFDSEY | L26; L14; L12; |
| | | | | L6 | | L18; L58; L52 |
| 3260 | SSTCMMCYK | L7 | AVIKTLQPV | L24; L3; | VVVLSFEL | L60; L59; L67; |
| | | | | L5 | | L68; L74; L61 |
| 3261 | SSTFNVPMEK | L7 | AVKMFDAYV | L13; L3; | VWKSYVHVV | L70; L3; L69; L39; |
| | | | | L58 | | L58; L51 |
| 3262 | SSVLHSTQ | L48 | AVLDMCASLK | L6; L7; | VYMPASWVM | L71; L66; L9; L72; |
| | | | | L13 | | L8; L62 |
| 3263 | SSVLHSTQD | L65 | AVPYNMRVI | L64; L57; | VYSFLPGVY | L66; L14; L12; |
| | | | | L10 | | L72; L9; L71 |
| 3264 | SSVLHSTQDLF | L52 | AVRDPQTLEI | L43; L10; | VYYPDKVF | L9; L72; L66; L71; |
| | | | | L6 | | L8; L62 |
| 3265 | SSVLNDIL | L67 | AWYTERSEK | L13; L7; | VYYPDKVFR | L15; L17; L9; L21; |
| | | | | L6 | | L8; L72 |
| 3266 | SSYSLFDMSK | L7 | AYCCNIVNV | L13; L62; | WCKDGHVETF | L10; L43; L26; |
| | | | | L72 | | L11; L52; L16 |
| 3267 | STASDTYAC | L7 | AYKDYLASG | L66; L72; | WEIVKFIST | L46; L45; L37; |
| | | | | L15 | | L42; L29; L38 |
| 3268 | STDGNKIADK | L7 | AYKIEELF | L9; L62; | WLIINLVQM | L4; L11; L26; L3; |
| | | | | L71 | | L2; L5 |
| 3269 | STEKSNIIRGW | L52 | AYKTFPPTE | L66; L9; | WLSYFIASF | L16; L26; L12; |
| | | | | L31 | | L43; L10; L27 |
| 3270 | STFISAARQGF | L16 | AYNMMISAGF | L9; L72; | WLTNIFGTV | L24; L3; L5; L2; |
| | | | | L71 | | L20; L4 |
| 3271 | STGSNVFQT | L7 | AYRKVLLRK | L13; L6; | WPWYIWLGF | L49; L31; L39; |
| | | | | L7 | | L50; L22; L32 |
| 3272 | STIQRKYK | L13 | AYVNTFSST | L72; L71; | YACWHHSI | L47; L70; L59; |
| | | | | L8 | | L67; L68; L61 |
| 3273 | STIQRKYKGI | L10 | CAPLTVFF | L62; L59; | YAYLRKHF | L59; L70; L47; |
| | | | | L74 | | 8L5; L60; L69 |
| 3274 | STKHFYWF | L58 | CAYCCNIV | L47; L70; | YAYLRKHFSM | L61; L60; L59; |
| | | | | L69 | | L69; L67; L70 |
| 3275 | STKHFYWFFSNY | L14 | CEESSAKSA | L38; L42; | YECDIPIGA | L46; L38; L42; |
| | | | | L46 | | L37; L50; L36 |
| 3276 | STNSRIKAS | L10 | CEIVGGQIVT | L36; L42; | YELQTPFEI | L45; L36; L37; |
| | | | | L38 | | L40; L41; L8 |
| 3277 | STQLGIEFLK | L7 | CEKALKYLP | L42; L38; | YFPLQSYGF | L62; L9; L72; L71; |
| | | | | L46 | | L12; L8 |
| 3278 | STQWSLFFFL | L20 | CELYHYQEC | L33; L45; | YIAQVDVVNF | L58; L74; L12; |
| | | | | L38 | | L26; L10; L11 |
| 3279 | STQYEYGTEDDY | L14 | CESHGKQV | L45; L48; | YIDINGNL | L68; L63; L1; L62; |
| | | | | L37 | | L74; L60 |
| 3280 | STSAFVETVK | L7 | CGVDAVNLL | L64; L69; | YIFFASFY | L58; L59; L12; L1; |
| | | | | L8 | | L43; L26 |
| 3281 | STSGRWVL | L70 | CIDGALLTK | L7; L6; | YIICISTKHF | L11; L10; L58; |
| | | | | 8L6 | | L43; L60; L26 |
| 3282 | STSHKLVLS | L20 | CINGLMLL | L74; L73; | YIRKLHDEL | L67; L58; L59; |
| | | | | L59 | | L74; L23; L60 |
| 3283 | SVAIKITEHSW | L52 | CLDDRCIL | L62; L63; | YKTPPIKDF | L27; L66; L60; L9; |
| | | | | L68 | | L58; L43 |
| 3284 | SVASQSIIA | L51 | CLFLLPSL | L59; L4; | YLCFLAFLL | L4; L5; L2; L74; |
| | | | | L32 | | L1; L64 |
| 3285 | SVCRLMKTI | L10 | CLLNRYFRL | L4; L23; | YLFQHANL | L74; L23; L3; L2; |
| | | | | L2 | | L95; L4 |
| 3286 | SVFNICQAVT | L20 | CNDPFLGVY | L18; L1; | YLVSTQEF | L43; L59; L26; |
| | | | | L41 | | L27; L49; L74 |
| 3287 | SVIDLLLD | L11 | CNDPFLGVYY | L1; L18; | YLYFIKGL | L23; L3; L74; L4; |
| | | | | L12 | | L34; L2 |
| 3288 | SVIDLLLDDFV | L20 | CNLGGAVCR | L17; L15; | YQHEETIYNL | L34; L44; L4; L24; |
| | | | | L21 | | L35; L3 |
| 3289 | SVLLFLAFVVF | L52 | CPAVAKHDF | L49; L31; | YQIGGYTEK | L43; L27; L26; |
| | | | | L39 | | L24; L7; L58 |
| 3290 | SVLYYQNNV | L4 | CSFGGVSV | L48; L70; | YQKVGMQKY | L26; L43; L58; |
| | | | | L69 | | L27; L14; L24 |
| 3291 | SVLYYQNNVF | L16 | CSHAAVDAL | L67; L61; | YQLYSTQL | L44; L27; L25; |
| | | | | L57 | | L35; L48; L28 |
| 3292 | SVMLTNDNTSRY | L1 | CSLSHRFYR | L15; L17; | YSHVVAFNTL | L60; L67; L61; |
| | | | | L21 | | L53; L59; L57 |
| 3293 | SVNITFELDER | L15 | CSMTDIAKK | L7; L13; | YSLLMPIL | L59; L74; L60; |
| | | | | L19 | | L61; L67; L47 |
| 3294 | SVPWDTIAN | L57 | CSTMTNRQF | L55; L54; | YSPIFLIV | L73; L47; L48; |
| | | | | L52 | | L59; L57; L67 |
| 3295 | SVRVLQKA | L51 | CTCGKQATKY | L11; L18; | YSTGSNVF | L59; L54; L58; |
| | | | | L14 | | L60; L70; L55 |
| 3296 | SVSPKLFIRQ | L20 | CTFEYVSQPF | L10; L16; | YTNSFTRGVY | L1; L14; L11; L10; |
| | | | | L53 | | L18; L12 |
| 3297 | SVSSPDAVTA | L51 | CTFTRSTNSR | L21; L19; | YVKPGGTSS | L43; L26; L58; |
| | | | | L15 | | L51; L11; L10 |
| 3298 | SVTVKNGSI | L10 | CTGSIPCSV | L20; L70; | YVVDDPCPIHF | L31; L60; L11; |
| | | | | L73 | | L12; L10; L58 |
| 3299 | SVTVKNGSIHLY | L14 | CVCSVIDLL | L20; L74; | YVWKSYVHV | L5; L24; L2; L20; |
| | | | | L5 | | L58; L63 |
| 3300 | SVVLLSVLQ | L7 | CVDTVRTNV | L63; L5; | YVYANGGKGF | L10; L11; L26; |
| | | | | L86 | | 8L5; L60; L12 |
| 3301 | SVVNARLRA | L51 | CVDTVRTNVY | L18; L1; | YYHTTDPSFL | L62; L72; L71; L9; |
| | | | | L14 | | L66; L8 |
| 3302 | SVVNIQKEIDR | L19 | CVRGTTVLLK | L6; L13; | YYNTTKGGRF | L8; L9; L71; L72; |
| | | | | L7 | | L62; L12 |
| 3303 | SVVSKVVKVT | L10 | CYFGLFCL | L72; L66; | YYPSARIV | L64; L71; L72; |
| | | | | L71 | | L66; L62; L47 |
| 3304 | SVVSKVVKVTI | L20 | CYFPLQSYGF | L8; L9; | AAGLEAPFL | L74; L61; L69; |
| | | | | L12 | | L70; L60; L63; L31 |
| 3305 | SVYAWNRKRI | L10 | DALCEKAL | L23; L25; | AAIMQLFF | L58; L74; L59; |
| | | | | L47 | | L69; L70; L54; L60 |
| 3306 | SVYPVASPNEC | L7 | DALCEKALKY | L31; L12; | ACPDGVKHVY | L14; L40; L71; |
| | | | | L11 | | L11; L41; L26; L66 |
| 3307 | SWFTALTQ | L48 | DAQSFLNRV | L47; L20; | AEASKKPRQ | L46; L36; L40; |
| | | | | L70 | | L42; L41; L45; L38 |
| 3308 | SWNLREMLA | L42 | DAYPLTKH | L47; L25; | AELEGIQYG | L40; L45; L41; |
| | | | | L11 | | L36; L42; L46; L37 |
| 3309 | SWQTGDFVK | L7 | DCLGDIAAR | L17; L19; | AEVQIDRL | L37; L36; L33; |
| | | | | L11 | | L29; L45; L41; L40 |
| 3310 | SWQTGDFVKA | L42 | DDLNQLTGY | L29; L11; | AFLLFLVL | L71; L72; L67; |
| | | | | L10 | | L59; L62; L23; L8 |
| 3311 | SYELQTPFEI | L9 | DEAGSKSP | L29; L46; | AFNTLLFLM | L62; L12; L9; L15; |
| | | | | L38 | | L71; L8; L72 |
| 3312 | SYFAVHFIS | L66 | DEFSSNVA | L25; L29; | AFVETVKGL | L72; L71; L8; L62; |
| | | | | L46 | | L41; L40; L9 |
| 3313 | SYFTEQPID | L65 | DEFSSNVANY | L29; L41; | AHIQWMVMF | L34; L28; L27; |
| | | | | L40 | | L35; L26; L8; L9 |
| 3314 | SYFVVKRHT | L72 | DELKINAA | L29; L25; | AIVMVTIML | L74; L44; L61; |
| | | | | L46 | | L60; L69; L5; L73 |
| 3315 | SYREAACC | L72 | DELKINAAC | L29; L40; | AIVSTIQRKY | L14; L10; L12; |
| | | | | L41 | | L40; L11; L41; L26 |
| 3316 | SYREAACCH | L72 | DERIDKVL | L29; L25; | ALDPLSETK | L5; L18; L1; L7; |
| | | | | L23 | | L2; L13; L6 |
| 3317 | SYTTTIKPVTY | L12 | DEVARDLSLQF | L41; L40; | ALDQAISMW | L18; L5; L1; L63; |
| | | | | L29 | | L68; L10; L16 |
| 3318 | SYVGCHNKC | L8 | DEWSMATYY | L29; L41; | ALLAVFQSA | L2; L46; L5; L4; |
| | | | | L40 | | L3; L24; L51 |
| 3319 | SYVGCHNKCAY | L71 | DFDTWFSQR | L17; L15; | ALSKGVHFV | L2; L3; L24; L4; |
| | | | | L19 | | L5; L64; L73 |
| 3320 | SYYCKSHKP | L8 | DFIDTKRGVY | L11; L10; | ALWEIQQV | L2; L24; L4; L3; |
| | | | | L14 | | L5; L48; L64 |
| 3321 | SYYSLLMPIL | L9 | DFNLVAMKY | L29; L12; | ASFSASTSAF | L26; L43; L16; |
| | | | | L14 | | L52; L10; L27; L54 |
| 3322 | TACSHAAV | L70 | DFQVTIAEI | L72; L62; | ASHMYCSFY | L18; L43; L14; |
| | | | | L71 | | L26; L1; L58; L70 |
| 3323 | TACTDDNALAY | L31 | DFTEERLKL | L8; L25; | ASLPFGWL | L74; L61; L73; |
| | | | | L9 | | L55; L59; L60; L67 |
| 3324 | TADIVVFDE | L68 | DFTEERLKLF | L9; L8; | ATATIPIQA | L7; L20; L69; L13; |
| | | | | L10 | | L3; L46; L42 |
| 3325 | TAFVTNVNA | L50 | DFVEIIKSQ | L10; L17; | ATVVIGTSKF | L52; L53; L10; |
| | | | | L11 | | L54; L11; L16; L26 |
| 3326 | TAGAAAYYV | L70 | DGISQYSLR | L19; L17; | AVAKHDFFKF | L12; L10; L11; |
| | | | | L11 | | L16; L53; L52; L54 |
| 3327 | TALLTLQQI | L47 | DGNKIADKY | L29; L14; | AYANRNRFL | L64; L72; L71; L9; |
| | | | | L11 | | L8; L66; L40 |
| 3328 | TALLTLQQIEL | L67 | DGVKHVYQL | L25; L29; | AYILFTRF | L9; L72; L8; L71; |
| | | | | L23 | | L62; L27; L12 |
| 3329 | TALRLCAY | L59 | DGYPLECI | L47; L25; | AYLRKHFSM | L71; L8; L72; L9; |
| | | | | L48 | | L66; L12; L33 |
| 3330 | TANKWDLI | L47 | DHSSSSDNI | L34; L28; | AYNVTQAF | L72; L71; L9; L62; |
| | | | | L35 | | L43; L33; L66 |
| 3331 | TAQNSVRV | L70 | DHSSSSDNIAL | L34; L28; | AYVDNSSL | L72; L71; L66; L8; |
| | | | | L35 | | L62; L9; L61 |
| 3332 | TASALGKL | L74 | DHVISTSHKL | L34; L35; | CANGQVFGL | L58; L70; L60; |
| | | | | L28 | | L32; L65; L69; L61 |
| 3333 | TASALGKLQ | L49 | DIAANTVIW | L10; L49; | CLTPVYSFL | L74; L5; L4; L2; |
| | | | | L19 | | L3; L61; L44 |
| 3334 | TATIPIQA | L50 | DIADTTDAV | L11; L20; | DAALALLL | L47; L74; L23; |
| | | | | L10 | | L25; L31; L67; L61 |
| 3335 | TATIPIQASL | L61 | DIADTTDAVR | L19; L11; | DAFKLNIKLL | L47; L32; L69; |
| | | | | L17 | | L10; L20; L74; L49 |
| 3336 | TAYANSVFNI | L47 | DIKDLPKEI | L10; L3; | DAPAHISTI | L47; L57; L10; |
| | | | | L23 | | L49; L70; L69; L11 |
| 3337 | TAYNGYLTSS | L43 | DKFTDGVCL | L44; L35; | DASGKPVPY | L49; L70; L58; |
| | | | | L25 | | L31; L69; L59; L43 |
| 3338 | TCASEYTGNY | L14 | DLATNNLVV | L2; L3; | DEVARDLSL | L25; L29; L41; |
| | | | | L11 | | L37; L36; L40; L23 |
| 3339 | TCATTRQV | L48 | DLDEWSMATYY | L1; L18; | DFSSEIIGY | L11; L12; L17; |
| | | | | L11 | | L10; L14; L29; L49 |
| 3340 | TCATTRQVV | L28 | DMFLGTCRR | L17; L19; | DFYDFAVSK | L17; L19; L13; |
| | | | | L21 | | L72; L71; L7; L6 |
| 3341 | TCDWTNAGDY | L1 | DMSKFPLKL | L73; L35; | DLKPVSEEV | L3; L23; L2; L20; |
| | | | | L74 | | L5; L43; L11 |
| 3342 | TCFSTASDTY | L14 | DMVPHISR | L25; L17; | DSAEVAVKM | L19; L20; L11; |
| | | | | L19 | | L53; L54; L29; L70 |
| 3343 | TCGKQATKY | L14 | DPFLGVYYH | L47; L29; | DTFCAGSTF | L10; L53; L11; |
| | | | | L31 | | L52; L16; L58; L54 |
| 3344 | TCTERLKL | L25 | DPQTLEIL | L23; L25; | EANMDQESF | L49; L43; L54; |
| | | | | L47 | | L31; L10; L52; L32 |
| 3345 | TDAQTGSSK | L19 | DQFKHLIPL | L25; L44; | EAVGTNLPL | L67; L61; L20; |
| | | | | L35 | | L32; L60; L74; L31 |
| 3346 | TDAVRDPQTL | L33 | DRAMPNMLR | L19; L17; | EEAIRHVRA | L42; L46; L38; |
| | | | | L30 | | L25; L40; L29; L37 |
| 3347 | TDFATSAC | L33 | DRYPANSIVCR | L19; L30; | EEAIRHVRAW | L40; L41; L10; |
| | | | | L17 | | L42; L38; L29; L46 |
| 3348 | TDFSRVSA | L46 | DSFKEELDKY | L10; L11; | EEIAIILAS | L41; L40; L42; |
| | | | | L14 | | L46; L29; L20; L37 |
| 3349 | TDFSSEIIGY | L27 | DTANPKTPK | L19; L7; | EEIAIILASF | L40; L41; L10; |
| | | | | L6 | | L11; L29; L37; L20 |
| 3350 | TDFVNEFY | L29 | DTDFVNEFY | L1; L18; | EEVVENPTI | L41; L45; L40; |
| | | | | L12 | | L36; L29; L37; L42 |
| 3351 | TDFVNEFYAY | L29 | DTKFKTEGL | L23; L10; | EHYVRITGL | L25; L23; L28; |
| | | | | L11 | | L35; L34; L10; L11 |
| 3352 | TDGNKIADKY | L14 | DTLKNLSDR | L17; L19; | EPKLGSLVV | L51; L39; L47; |
| | | | | L11 | | L50; L22; L23; L42 |
| 3353 | TDLEGNFY | L29 | DTVRTNVY | L29; L11; | EQWNLVIGF | L27; L26; L34; |
| | | | | L10 | | L11; L10; L30; L24 |
| 3354 | TDLMAAYV | L33 | DTVRTNVYL | L20; L11; | EVTPSGTWL | L11; L10; L20; |
| | | | | L53 | | L74; L61; L60; L28 |
| 3355 | TDLTKPYI | L33 | DTYACWHHSI | L10; L25; | EYPIIGDEL | L9; L72; L71; L8; |
| | | | | L20 | | L66; L62; L57 |
| 3356 | TDQSSYIV | L33 | DVSSAINR | L19; L17; | FACVVADAV | L67; L59; L61; |
| | | | | L21 | | L50; L47; L70; L68 |
| 3357 | TDYKHWPQIA | L46 | DVSSAINRP | L11; L10; | FAFACPDGV | L58; L69; L47; |
| | | | | L19 | | L70; L20; L50; L67 |
| 3358 | TECSNLLLQ | L41 | DVTDVTQLYL | L20; L11; | FANKHADF | L59; L58; L74; |
| | | | | L10 | | L63; L60; L49; L70 |
| 3359 | TEDDYQGKPLEF | L41 | DVVAIDYKH | L11; L20; | FASEAARV | L70; L69; L47; |
| | | | | L19 | | L58; L63; L50; L64 |
| 3360 | TEERLKLFDRY | L41 | DVVAIDYKHY | L10; L11; | FAVHFISN | L59; L61; L67; |
| | | | | L14 | | L50; L60; L69; L70 |
| 3361 | TEEVGHTD | L45 | DVVNQNAQAL | L10; L20; | FAVHFISNSW | L49; L10; L54; |
| | | | | L11 | | L61; L52; L69; L31 |
| 3362 | TEEVGHTDLM | L36 | DVVQEGVLT | L19; L11; | FAWWTAFV | L47; L70; L58; |
| | | | | L20 | | L59; L50; L69; L68 |
| 3363 | TEEVGHTDLMA | L38 | DVVRQCSGV | L11; L10; | FAWWTAFVT | L69; L32; L59; |
| | | | | L20 | | L67; L31; L50; L70 |
| 3364 | TEEVVLKT | L45 | DYTEISFML | L9; L8; | FFASFYYVW | L9; L8; L10; L12; |
| | | | | L66 | | L69; L54; L49 |
| 3365 | TEEVVLKTG | L38 | EAVEAPLV | L47; L20; | FFFLYENAF | L71; L72; L62; |
| | | | | L48 | | L59; L9; L12; L31 |
| 3366 | TEGLCVDI | L45 | EAVKTQFNY | L49; L11; | FIDTKRGVY | L1; L18; L58; L14; |
| | | | | L12 | | L63; L12; L68 |
| 3367 | TEGLCVDIPGI | L41 | EAYEQAVAN | L17; L19; | FIQQKLAL | L23; L59; L60; |
| | | | | L32 | | L61; L67; L74; L68 |
| 3368 | TEHSWNAD | L46 | ECAQVLSEM | L11; L10; | FITESKPSV | L2; L5; L4; L3; |
| | | | | L20 | | L61; L50; L58 |
| 3369 | TEIYQAGST | L45 | ECIKDLLAR | L17; L19; | FLARGIVFM | L74; L2; L3; L4; |
| | | | | L11 | | L69; L64; L11 |
| 3370 | TEKSNIIRG | L45 | EDDNLIDSY | L1; L18; | FLGRYMSAL | L57; L3; L74; L2; |
| | | | | L29 | | L4; L67; L61 |
| 3371 | TEKWESGV | L45 | EEAALCTF | L29; L41; | FLPGVYSV | L5; L57; L2; L3; |
| | | | | L33 | | L64; L4; L62 |
| 3372 | TELEPPCR | L29 | EEAKKVKP | L42; L46; | FPQSAPHGV | L50; L47; L32; |
| | | | | L38 | | L51; L39; L2; L22 |
| 3373 | TEMLAKALR | L40 | EEAKKVKPTV | L42; L37; | FPQSAPHGVVF | L31; L39; L22; |
| | | | | L45 | | L56; L50; L32; L49 |
| 3374 | TEMLAKALRKV | L45 | EEFEPSTQ | L29; L48; | FTIGTVTLK | L7; L19; L6; L17; |
| | | | | L25 | | L13; L58; L11 |
| 3375 | TENLTKEG | L29 | EEFEPSTQYEY | L41; L40; | FTTVDNINL | L53; L60; L68; |
| | | | | L29 | | L61; L20; L70; L58 |
| 3376 | TERSEKSY | L29 | EEKFKEGV | L45; L42; | FVDDIVKTD | L5; L68; L49; L32; |
| | | | | L23 | | L63; L60; L58 |
| 3377 | TETDLTKG | L45 | EEMLDNRAT | L40; L42; | FVDSDVETK | L68; L49; L63; L5; |
| | | | | L41 | | L67; L1; L7 |
| 3378 | TEVNEFAC | L29 | EEQEEDWLD | L42; L40; | FVFLVLLPLV | L20; L50; L47; L5; |
| | | | | L38 | | L2; L3; L4 |
| 3379 | TEVPVAIH | L29 | EERLKLFDRY | L29; L40; | FYLCFLAF | L72; L71; L9; L59; |
| | | | | L41 | | L66; L8; L62 |
| 3380 | TEVVGDIILKP | L38 | EETFKLSY | L29; L41; | GAMDTTSY | L43; L59; L27; |
| | | | | L40 | | L26; L58; L70; L18 |
| 3381 | TFDSEYCR | L62 | EETGLLMPL | L41; L40; | GETLGVLV | L45; L48; L37; |
| | | | | L20 | | L36; L38; L46; L33 |
| 3382 | TFDSEYCRH | L62 | EETIYNLL | L29; L25; | GETLGVLVP | L46; L38; L42; |
| | | | | L45 | | L37; L45; L36; L41 |
| 3383 | TFEEAALC | L62 | EETIYNLLK | L42; L41; | GEVPVSIIN | L36; L37; L46; |
| | | | | L40 | | L38; L29; L45; L41 |
| 3384 | TFELDERI | L62 | EEVGHTDL | L25; L29; | GLEAPFLYL | L5; L6; L2; L1; |
| | | | | L37 | | L18; L4; L74 |
| 3385 | TFFIYNKI | L48 | EEVLSEARQH | L41; L40; | GLLPPKNSI | L24; L2; L4; L16; |
| | | | | L29 | | L23; L44; L5 |
| 3386 | TFFIYNKIV | L48 | EFLTRNPAW | L9; L71; | GSKSPIQY | L55; L70; L43; |
| | | | | L8 | | L26; L13; L58; L27 |
| 3387 | TFFIYNKIVD | L65 | EHVNNSYEC | L35; L34; | GSLPINVIV | L73; L24; L69; |
| | | | | L28 | | L52; L20; L70; L2 |
| 3388 | TFFKLVNK | L17 | EIDFLELAM | L68; L1; | GVDIAANTV | L68; L63; L5; L28; |
| | | | | L67 | | L18; L24; L45 |
| 3389 | TFFPDLNGD | L8 | EIDPKLDNYY | L1; L11; | GVKDCVVL | L23; L39; L67; |
| | | | | 1L8 | | L26; L44; L56; L13 |
| 3390 | TFHLDGEV | L62 | EILDITPCSF | L10; L11; | GYQPYRVVVL | L72; L71; L65; |
| | | | | L16 | | L66; L9; L8; L44 |
| 3391 | TFHLDGEVITF | L8 | EIPRRNVATL | L10; L57; | HEHEIAWYT | L29; L37; L38; |
| | | | | L11 | | L45; L41; L40; L36 |
| 3392 | TFKVSIWNLDY | L12 | EIVGGQIVT | L20; L10; | HHWLLLTI | L48; L34; L35; |
| | | | | L19 | | L47; L25; L28; L24 |
| 3393 | TFLKKDAPYI | L8 | EIVKFISTC | L10; L11; | HPTQAPTHL | L56; L32; L22; |
| | | | | L19 | | L49; L39; L31; L50 |
| 3394 | TFNGECPNFV | L62 | EKTHVQLSL | L44; L35; | HSYGADLKSF | L10; L53; L52; |
| | | | | L20 | | L43; L26; L58; L54 |
| 3395 | TFNGECPNFVF | L8 | ELAMDEFIER | L19; L17; | HTTDPSFLGR | L19; L17; L21; |
| | | | | L21 | | L15; L7; L11; L6 |
| 3396 | TFNVPMEK | L62 | ELEGIQYGR | L17; L19; | HVGEIPVAYR | L19; L21; L17; |
| | | | | L21 | | L15; L11; L20; L6 |
| 3397 | TFSSTFNVP | L8 | ELPTGVHAG | L11; L57; | IAIAMACL | L61; L60; L59; |
| | | | | L10 | | L74; L70; L67; L47 |
| 3398 | TFTYASALW | L9 | ELSPVALR | L19; L17; | IAIVMVTI | L47; L48; L70; |
| | | | | L21 | | L59; L68; L61; L69 |
| 3399 | TFVTHSKGLYR | L17 | ELVIGAVILR | L19; L17; | IAKKPTETI | L43; L70; L49; |
| | | | | L21 | | L47; L53; L52; L58 |
| 3400 | TGDFVKATC | L68 | ELYSPIFL | L74; L23; | IATVREVL | L59; L61; L60; |
| | | | | L20 | | L67; L68; L70; L23 |
| 3401 | TGDLQPLEQ | L68 | ELYSPIFLIV | L20; L11; | IAWNSNNL | L47; L68; L60; |
| | | | | L24 | | L61; L63; L70; L74 |
| 3402 | TGDSCNNY | L1 | ENPDILRV | L48; L11; | IEVNSFSGY | L29; L41; L40; |
| | | | | L20 | | L46; L26; L27; L45 |
| 3403 | TGDSCNNYM | L63 | EPEEHVQIH | L49; L32; | IEYPIIGDEL | L36; L67; L37; |
| | | | | L31 | | L29; L45; L41; L40 |
| 3404 | TGKIADYNY | L14 | EPEEHVQIHTI | L49; L39; | IFLEGETL | L72; L71; L8; L9; |
| | | | | L32 | | L62; L67; L25 |
| 3405 | TGLLMPLKA | L50 | EPEYFNSV | L50; L23; | IIIGGAKL | L60; L74; L61; |
| | | | | L47 | | L63; L58; L67; L59 |
| 3406 | TGLYPTLNI | L8 | EQPYVFIKR | L19; L17; | IINLIIKNL | L60; L4; L39; L64; |
| | | | | L15 | | L73; L58; L5 |
| 3407 | TGNTLQCI | L48 | EQYVFCTV | L48; L24; | IPTNFTISV | L50; L51; L47; |
| | | | | L25 | | L39; L22; L32; L56 |
| 3408 | TGNYQCGHY | L14 | ERDISTEI | L35; L25; | IPVAYRKVL | L39; L32; L22; |
| | | | | L34 | | L56; L31; L47; L25 |
| 3409 | TGQAITVTP | L38 | ERLKLFDRY | L30; L14; | ISDYDYYRY | L1; L18; L54; L14; |
| | | | | L12 | | L53; L52; L49 |
| 3410 | TGRLQSLQTY | L26 | ERYKLEGYAF | L30; L35; | ITLATCELY | L18; L12; L52; L1; |
| | | | | L34 | | L54; L14; L16 |
| 3411 | TGYRVTKN | L48 | ESGLKTILR | L17; L19; | ITREVGFVV | L13; L20; L53; |
| | | | | L21 | | L70; L73; L58; L39 |
| 3412 | THDVSSAINR | L34 | ESLVPGFNEK | L19; L17; | IVDTVSALV | L5; L63; L62; L68; |
| | | | | L7 | | L18; L1; L20 |
| 3413 | THDVSSAINRP | L34 | ESNKKFLPF | L10; L11; | KAGGTTEML | L74; L67; L54; |
| | | | | L73 | | L58; L70; L65; L63 |
| 3414 | THGLNLEEA | L28 | ESVQTFFKL | L20; L10; | KCSAYTVEL | L44; L56; L73; |
| | | | | L11 | | L65; L66; L16; L39 |
| 3415 | THSDKFTDG | L35 | ETAQNSVRV | L20; L11; | KEGSSVEL | L37; L33; L36; |
| | | | | L10 | | L45; L65; L56; L44 |
| 3416 | THWFVTQRN | L34 | ETAQNSVRVL | L10; L20; | KEIKESVQT | L38; L37; L36; |
| | | | | L11 | | L46; L42; L45; L44 |
| 3417 | THWFVTQRNF | L34 | ETFKLSYGI | L20; L10; | KESPFELED | L65; L38; L42; |
| | | | | L11 | | L45; L46; L36; L37 |
| 3418 | TIAEILLIIM | L20 | ETGTLIVNS | L17; L20; | KFLTENLLLY | L12; L14; L8; L18; |
| | | | | L19 | | L13; L6; L52 |
| 3419 | TIAKNTVKSV | L3 | ETIYNLLKD | L11; L20; | KKNNLPFKL | L44; L66; L73; |
| | | | | L10 | | L16; L64; L27; L4 |
| 3420 | TIANYAKPF | L43 | ETKAIVSTIQR | L17; L19; | KLFAAETL | L55; L16; L54; |
| | | | | L21 | | L44; L24; L23; L4 |
| 3421 | TIATYCTGSI | L10 | ETKCTLKSF | L10; L11; | KLFDRYFKYW | L16; L6; L52; L3; |
| | | | | L52 | | L13; L21; L10 |
| 3422 | TIAYIICISTK | L19 | ETKFLTENL | L20; L11; | KLMGHFAWW | L16; L54; L6; L21; |
| | | | | L10 | | L10; L3; L14 |
| 3423 | TIDGSSGVVN | L68 | ETLKATEETF | L53; L52; | KNTCDGTTF | L53; L54; L55; L9; |
| | | | | L10 | | L26; L27; L8 |
| 3424 | TIEYPIIGDEL | L67 | ETLPTEVL | L25; L23; | KPREQIDGY | L14; L26; L49; |
| | | | | L20 | | L27; L31; L18; L16 |
| 3425 | TIGPDMFL | L74 | ETSNSFDVL | L20; L10; | KRGDKSVYY | L65; L66; L30; |
| | | | | L67 | | L64; L14; L12; L18 |
| 3426 | TIKKPNELSR | L17 | EVAKNLNES | L20; L10; | KSREETGLL | L55; L53; L13; |
| | | | | L11 | | L73; L14; L54; L52 |
| 3427 | TIKVFTTVD | L43 | EVAKNLNESL | L20; L10; | KTFPPTEPK | L13; L6; L7; L16; |
| | | | | L11 | | L21; L54; L52 |
| 3428 | TILGSALL | L74 | EVARDLSLQ | L20; L11; | KTSVDCTMY | L18; L53; L54; |
| | | | | L10 | | L14; L16; L52; L1 |
| 3429 | TILRKGGRTI | L10 | EVARDLSLQF | L11; L10; | KVQIGEYTF | L16; L53; L54; |
| | | | | L20 | | L26; L55; L52; L14 |
| 3430 | TILTSLLV | L48 | EVFAQVKQIY | L11; L10; | KVYYGNAL | L67; L59; L55; |
| | | | | L12 | | L60; L56; L61; L71 |
| 3431 | TILTSLLVLV | L4 | EVGPEHSL | L20; L67; | KYDFTEERL | L62; L63; L9; L8; |
| | | | | L23 | | L68; L34; L36 |
| 3432 | TIQRKYKGI | L10 | EVGPEHSLAEY | L11; L14; | LADKFPVLH | L63; L74; L68; |
| | | | | L10 | | L49; L32; L62; L1 |
| 3433 | TISVTTEI | L48 | EVLSEARQH | L11; L17; | LAHIQWMVM | L70; L61; L59; |
| | | | | L10 | | L32; L47; L69; L31 |
| 3434 | TITVEELKK | L7 | EVNSFSGYL | L11; L20; | LASHMYCSF | L43; L49; L32; |
| | | | | L10 | | L59; L58; L31; L70 |
| 3435 | TIYNLLKD | L48 | EVNSFSGYLK | L19; L17; | LAVFDKNLY | L31; L49; L12; |
| | | | | L7 | | L52; L1; L18; L58 |
| 3436 | TIYSLLLC | L48 | EVQELYSPI | L10; L20; | LAWLYAAVI | L47; L68; L67; |
| | | | | L11 | | L31; L50; L70; L59 |
| 3437 | TKAIVSTI | L48 | EVTGDSCNNY | L11; L10; | LCLTPVYSF | L8; L9; L52; L12; |
| | | | | L14 | | L31; L16; L65 |
| 3438 | TKAIVSTIQR | L19 | EVVENPTIQKD | L11; L19; | LEILQKEKV | L45; L38; L37; |
| | | | | L10 | | L46; L29; L36; L42 |
| 3439 | TKFKTEGL | L27 | EVVGDIIL | L74; L20; | LEIPRRNV | L45; L48; L38; |
| | | | | L67 | | L37; L29; L46; L25 |
| 3440 | TKFLTENLLL | L44 | EVVLKKLKK | L19; L11; | LESELVIGA | L42; L46; L38; |
| | | | | L7 | | L37; L29; L41; L50 |
| 3441 | TKFLTENLLLY | L27 | EVVLKTGDL | L11; L10; | LFLAFVVF | L72; L9; L71; L62; |
| | | | | L20 | | L8; L59; L47 |
| 3442 | TKGGRFVL | L66 | EWFLAYIL | L25; L23; | LHKPIVWHV | L3; L28; L64; L69; |
| | | | | L35 | | L70; L58; L4 |
| 3443 | TKGSLPINV | L64 | EWSMATYYL | L20; L34; | LHPDSATL | L57; L28; L35; |
| | | | | L9 | | L66; L71; L72; L62 |
| 3444 | TKGTLEPEY | L66 | EYADVFHLYL | L8; L9; | LLDDFVEI | L5; L68; L62; L63; |
| | | | | L20 | | L2; L1; L67 |
| 3445 | TKHFYWFFSNY | L27 | EYCPIFFIT | L8; L9; | LLDKRTTCF | L63; L62; L1; L5; |
| | | | | L17 | | L74; L68; L49 |
| 3446 | TKIKPHNSH | L27 | EYKGPITDVF | L9; L8; | LLNKHIDAY | L26; L43; L18; |
| | | | | L10 | | L14; L12; L1; L16 |
| 3447 | TKMSECVL | L44 | FACPDGVKHVY | L31; L52; | LLTPLGIDL | L74; L56; L5; L61; |
| | | | | L49 | | L2; L4; L64 |
| 3448 | TKNSKVQIGEY | L14 | FADDLNQLTGY | L1; L18; | LLYDANYFL | L2; L4; L5; L61; |
| | | | | L31 | | L3; L74; L68 |
| 3449 | TKRFDNPVL | L44 | FAFPFTIYSLLL | L61; L31; | LPFAMGIIA | L50; L51; L31; |
| | | | | L74 | | L47; L56; L32; L22 |
| 3450 | TKTSVDCTM | L32 | FAMGIIAMS | L58; L69; | LPFGWLIVG | L50; L31; L47; |
| | | | | L70 | | L51; L32; L56; L39 |
| 3451 | TKTSVDCTMY | L27 | FAMGIIAMSAF | L31; L58; | LPFGWLIVGV | L50; L51; L47; |
| | | | | L61 | | L56; L31; L3; L20 |
| 3452 | TKYTMADLVY | L27 | FAMMFVKHK | L69; L70; | LPIGINITRF | L31; L49; L32; |
| | | | | L59 | | L11; L50; L56; L39 |
| 3453 | TLACFVLAAVY | L14 | FAQDGNAA | L50; L59; | LPTGTLLVD | L49; L51; L32; |
| | | | | L61 | | L50; L31; L65; L56 |
| 3454 | TLADAGFIKQ | L2 | FAQVKQIY | L59; L70; | LRANSAVKL | L64; L35; L66; |
| | | | | L31 | | L30; L34; L28; L25 |
| 3455 | TLAILTALRL | L4 | FARTRSMWSF | L58; L43; | LRPDTRYVL | L66; L65; L64; |
| | | | | L59 | | L35; L57; L5; L25 |
| 3456 | TLDNQDLN | L1 | FASVYAWNR | L17; L19; | LSDRELHL | L63; L1; L68; L74; |
| | | | | L15 | | L60; L62; L67 |
| 3457 | TLEETKFLT | L1 | FAVHFISNSWL | L67; L61; | LSLPVLQVR | L15; L52; L17; |
| | | | | L60 | | L55; L21; L53; L7 |
| 3458 | TLETAQNSV | L2 | FCALILAY | L59; L1; | LVIGAVILR | L19; L21; L7; L17; |
| | | | | L31 | | L15; L6; L20 |
| 3459 | TLKEILVTYN | L3 | FCDLKGKYV | L62; L63; | LVSDIDITFL | L20; L5; L4; L60; L |
| | | | | L68 | | 31; L74; L3 |
| 3460 | TLKGVEAVM | L14 | FCGVDAVNL | L74; L62; | LYYPSARI | L47; L9; L62; L48; |
| | | | | L34 | | L8; L71; L72 |
| 3461 | TLKQGEIKDA | L3 | FCLEASFNYL | L60; L61; | LYYPSARIVY | L71; L72; L12; |
| | | | | L67 | | L27; L66; L14; L31 |
| 3462 | TLKSFTVE | L23 | FCNDPFLGVY | L14; L31; | MAAYVDNSSL | L61; L32; L67; |
| | | | | L59 | | L31; L70; L20; L60 |
| 3463 | TLLALHRSYL | L4 | FCSQHTMLV | L64; L73; | MADSNGTITV | L63; L32; L68; |
| | | | | L58 | | L49; L70; L50; L18 |
| 3464 | TLLFLMSF | L23 | FDNLKTLL | L33; L23; | MEVTPSGTW | L40; L41; L46; |
| | | | | L62 | | L29; L36; L49; L45 |
| 3465 | TLLLQLCTFTR | L21 | FEHIVYGDF | L29; L41; | MFVKHKHAF | L71; L8; L72; L49; |
| | | | | L40 | | L9; L33; L29 |
| 3466 | TLLTKGTL | L23 | FEYYHTTDP | L46; L38; | MHANYIFWR | L19; L17; L21; |
| | | | | L42 | | L34; L15; L7; L35 |
| 3467 | TLNDFNLVAMK | L6 | FFAQDGNAA | L72; L71; | MLDMYSVML | L5; L62; L68; L63; |
| | | | | L50 | | L74; L2; L1 |
| 3468 | TLPKGFYAE | L57 | FFFAQDGNAA | L71; L72; | MLVYCFLGY | L12; L6; L26; L10; |
| | | | | L50 | | L11; L1; L18 |
| 3469 | TLPKGIMM | L57 | FFFLYENAFL | L72; L62; | MSNLGMPSY | L59; L43; L1; L18; |
| | | | | L71 | | L14; L52; L70 |
| 3470 | TLPKGIMMN | L57 | FFKLVNKF | L62; L72; | MVMCGGSLY | L11; L14; L26; |
| | | | | L71 | | L18; L10; L1; L12 |
| 3471 | TLPTEVLTEEV | L5 | FFKLVNKFL | L62; L72; | MVTNNTFTL | L20; L60; L16; |
| | | | | L71 | | L32; L61; L56; L10 |
| 3472 | TLVKQLSSN | L10 | FFLYENAF | L71; L72; | NAGIVGVLTL | L67; L31; L32; |
| | | | | L62 | | L49; L69; L61; L68 |
| 3473 | TLVPQEHY | L26 | FFPDLNGD | L71; L62; | NAQALNTL | L70; L59; L25; |
| | | | | L72 | | L67; L47; L60; L23 |
| 3474 | TLVTMPLGYV | L3 | FFPDLNGDVV | L72; L71; | NATRFASVY | L70; L31; L69; |
| | | | | L62 | | L58; L59; L14; L49 |
| 3475 | TLYCIDGALL | L2 | FFSNVTWFH | L72; L12; | NCYLATAL | L25; L23; L71; |
| | | | | L71 | | L72; L28; L67; L35 |
| 3476 | TMADLVYA | L3 | FFTYICGF | L62; L72; | NFGAISSVL | L72; L71; L8; L9; |
| | | | | L71 | | L28; L35; L62 |
| 3477 | TMLFTMLR | L17 | FFYVLGLAAI | L71; L72; | NFNQHEVLL | L62; L8; L71; L9; |
| | | | | L62 | | L34; L64; L72 |
| 3478 | TMLVKQGDDY | L14 | FGDDTVIE | L62; L63; | NLLLLFVTV | L2; L4; L5; L24; |
| | | | | L68 | | L50; L23; L3 |
| 3479 | TMPLGYVTH | L57 | FGPLVRKI | L47; L48; | NLVAVPTGY | L12; L26; L6; L14; |
| | | | | L64 | | L10; L27; L11 |
| 3480 | TNDNTSRYW | L49 | FGWLIVGV | L47; L48; | NPAWRKAVF | L39; L49; L23; |
| | | | | L50 | | L22; L31; L32; L56 |
| 3481 | TNDVSFLAH | L68 | FHLDGEVI | L35; L34; | NTCDGTTFTY | L11; L18; L10; L1; |
| | | | | L28 | | L16; L14; L12 |
| 3482 | TNGDFLHF | L65 | FIAGLIAIVM | L69; L58; | NTVKSVGKF | L11; L10; L53; |
| | | | | L67 | | L54; L58; L49; L52 |
| 3483 | TNGTKRFD | L65 | FIASFRLFAR | L17; L19; | NVVIKVCEF | L10; L11; L23; |
| | | | | L21 | | L49; L26; L58; L29 |
| 3484 | TNGVGYQPY | L43 | FIEDLLFNK | L7; L19; | NVVTTKIAL | L23; L32; L20; |
| | | | | 6L | | L67; L56; L60; L74 |
| 3485 | TNIFGTVY | L26 | FIKQYGDCL | L58; L67; | NYAKPFLNK | L13; L17; L7; L8; |
| | | | | L60 | | L9; L6; L19 |
| 3486 | TNIFGTVYEK | L19 | FIRQEEVQEL | L3; L58; | PSKPSKRSF | L43; L55; L52; |
| | | | | L10 | | L65; L26; L53; L58 |
| 3487 | TNMFTPLI | L48 | FKEGVEFL | L62; L63; | PYRVVVLSF | L9; L72; L66; L8; |
| | | | | L68 | | L65; L71; L43 |
| 3488 | TNNVAFQTV | L73 | FKLNIKLL | L66; L64; | QAVGACVL | L67; L70; L61; |
| | | | | L44 | | L69; L74; L60; L55 |
| 3489 | TNSFTRGVY | L14 | FKVSIWNL | L66; L74; | QEFKPRSQM | L29; L36; L45; |
| | | | | L44 | | 6L4; L37; L33; L41 |
| 3490 | TNSPRRAR | L17 | FLELAMDEF | L62; L1; | QEYADVFHL | L36; L37; L41; |
| | | | | L68 | | L40; L29; L33; L45 |
| 3491 | TNSSPDDQI | L34 | FLFVAAIF | L59; L62; | QFAPSASAF | L72; L71; L9; L28; |
| | | | | L74 | | L62; L12; L8 |
| 3492 | TNVLEGSVA | L46 | FLHFLPRV | L2; L4; | QLSLPVLQV | L4; L3; L24; L2; |
| | | | | L3 | | L5; L28; L73 |
| 3493 | TPAFDKSA | L51 | FLHVTYVPA | L43; L2; | QSADAQSF | L55; L54; L43; |
| | | | | L05 | | L53; L70; L52; L59 |
| 3494 | TPEANMDQES | L32 | FLKRGDKSVY | L43; L26; | QSRNLQEF | L59; L43; L55; |
| | | | | L14 | | L53; L26; L58; L52 |
| 3495 | TPGDSSSGW | L49 | FLLFLVLIML | L2; L4; | QTFFKLVNK | L7; L13; L6; L19; |
| | | | | L74 | | L15; L21; L17 |
| 3496 | TPGSSRGTSP | L51 | FLLLSVCL | L74; L23; | QWSLFFFLY | L12; L40; L41; L1; |
| | | | | L62 | | L18; L29; L14 |
| 3497 | TPKDHIGT | L51 | FLQSINFV | L2; L4; | RAKVGILCI | L24; L70; L69; |
| | | | | L3 | | L53; L55; L52; L49 |
| 3498 | TPKDHIGTRNPA | L51 | FLQSINFVRI | L5; L2; | RELGVVHNQ | L33; L36; L37; |
| | | | | L4 | | L41; L40; L38; L42 |
| 3499 | TPKYKFVR | L23 | FLRDGWEI | L43; L59; | REVGFVVP | L38; L46; L37; |
| | | | | L2 | | L42; L45; L29; L65 |
| 3500 | TPKYKFVRIQ | L39 | FLTWICLL | L74; L64; | RFDNPVLPF | L62; L8; L9; L72; |
| | | | | L23 | | L71; L12; L68 |

TABLE 1B
Peptides and Alleles

| | Set 1 | Set 1 | Set 2 | Set 2 |
|---|---|---|---|---|
| | Peptide | Allele | Peptide | Allele |
| 1 | RFLYIIKLI | L8; L24; L48; L9; L55; | NLSDRVVFV | L2; L3; L4; L64; L5; L20; L23; L69; |
| | | L13; L40 | | L24; L73 |
| 2 | RLQSLQTY | L26; L14; L18; L16; L27; | NLYDKLVSSF | L10; L11; L16; L26; L43; L71; L3; |
| | | L55; L24 | | L72; L12; L27 |
| 3 | RLRAKHYVY | L26; L18; L16; L14; L27; | NNAAIVLQL | L20; L35; L73; L64; L28; L69; L3; |
| | | L13; L12 | | L34; L31; L41 |
| 4 | RLYECLYRN | L24; L4; L16; L6; L2; L3; | NRFLYIIKL | L35; L64; L66; L25; L30; L65; L34; |
| | | L21 | | L29; L69; L28 |
| 5 | RNIKPVPEV | L73; L24; L13; L4; L3; | NRFNVAITR | L19; L30; L66; L35; L17; L25; L34; |
| | | L51; L48 | | L21; L15; L64 |
| 6 | RTILGSAL | L60; L55; L61; L59; L71; | NYNYLYRLF | L8; L9; L66; L62; L64; L71; L12; |
| | | L72; L54 | | L72; L40; L41 |
| 7 | RVFSAVGNICY | L16; L14; L6; L18; L52; | PTDNYITTY | L18; L1; L10; L11; L16; L53; L54; |
| | | L54; L7 | | L14; L58; L12 |
| 8 | RVVRSIFSR | L15; L21; L7; L19; L17; | QAWQPGVAM | L61; L31; L67; L32; L68; L43; L59; |
| | | L6; L16 | | L22; L70; L63 |
| 9 | RVYANLGER | L21; L15; L6; L7; L17; | QEILGTVSW | L40; L41; L29; L42; L38; L45; L46; |
| | | L19; L14 | | L36; L37; L52 |
| 10 | RYWEPEFY | L72; L71; L65; L14; L62; | QLIKVTLVF | L26; L9; L27; L16; L8; L11; L12; |
| | | L66; L12 | | L10; L43; L74 |
| 11 | SAFFGMSRI | L47; L70; L69; L49; L24; | QLYLGGMSY | L43; L26; L27; L6; L14; L1; L16; |
| | | L20; L10 | | L18; L12; L11 |
| 12 | SASKIITLK | L19; L7; L6; L17; L58; | QPYRVVVL | L56; L47; L39; L22; L23; L32; L25; |
| | | L49; L13 | | L48; L65; L31 |
| 13 | SEARQHLKD | L42; L41; L46; L45; L37; | QSADAQSFL | L73; L70; L20; L58; L61; L60; L53; |
| | | L38; L29 | | L69; L74; L55 |
| 14 | SEDKRAKV | L45; L37; L33; L38; L63; | QTIEVNSF | L54; L55; L11; L53; L10; L52; L29; |
| | | L42; L29 | | L26; L70; L72 |
| 15 | SEFRVYSSA | L46; L42; L38; L37; L29; | RIIPARARV | L73; L24; L3; L5; L4; L13; L20; |
| | | L41; L45 | | L14; L16; L58 |
| 16 | SEKQVEQKI | L45; L37; L24; L36; L41; | RLFARTRSM | L16; L33; L27; L21; L3; L6; L24; |
| | | L40; L42 | | L13; L14; L26 |
| 17 | SELVIGAV | L33; L29; L48; L37; L45; | RLITGRLQSL | L3; L4; L16; L24; L44; L2; L10; |
| | | L25; L46 | | L56; L26; L53 |
| 18 | SELVIGAVIL | L36; L37; L40; L41; L29; | RQEEVQELY | L27; L18; L26; L14; L1; L54; L16; |
| | | L44; L45 | | L24; L12; L44 |
| 19 | SEYKGPITD | L37; L45; L46; L29; L36; | RQWLPTGTL | L44; L27; L26; L24; L16; L68; L33; |
| | | L42; L38 | | L56; L22; L48 |
| 20 | SFKKGAKLL | L8; L13; L39; L72; L71; | RTTNGDFLHF | L16; L52; L53; L54; L18; L10; L8; |
| | | L9; L62 | | L12; L73; L9 |
| 21 | SHVVAFNTL | L34; L28; L35; L60; L44; | RVVVLSFEL | L53; L56; L44; L16; L54; L60; L73; |
| | | L61; L8 | | L55; L20; L65 |
| 22 | SLLSKGRLI | L4; L24; L55; L2; L8; L23; | RVYSTGSNVF | L16; L54; L26; L27; L53; L65; L52; |
| | | L16 | | L11; L8; L14 |
| 23 | SLRPDTRYV | L3; L13; L2; L4; L43; L64; | RYLALYNKY | L14; L12; L8; L13; L71; L72; L16; |
| | | L5 | | L9; L30; L27 |
| 24 | SPDDQIGYY | L49; L31; L1; L18; L14; | SAGFSLWVY | L58; L59; L69; L31; L14; L70; L43; |
| | | L12; L11 | | L12; L18; L52 |
| 25 | SPSGVYQCA | L51; L49; L39; L56; L50; | SASVYYSQL | L70; L60; L61; L58; L74; L67; L57; |
| | | L22; L32 | | L69; L59; L43 |
| 26 | SQSIIAYTM | L24; L28; L35; L26; L34; | SEYDYVIFT | L42; L46; L45; L37; L41; L38; L36; |
| | | L44; L29 | | L29; L40; L69 |
| 27 | SRLSFKELL | L30; L34; L35; L65; L25; | SIKNFKSVL | L23; L58; L43; L39; L26; L56; L69; |
| | | L64; L66 | | L3; L13; L10 |
| 28 | SRVLGLKTL | L25; L64; L30; L35; L66; | SLINTLNDL | L4; L3; L2; L74; L5; L24; L8; L57; |
| | | L34; L28 | | L26; L11 |
| 29 | STTTNIVTR | L19; L21; L7; L17; L15; | SLYVNKHAF | L43; L26; L16; L23; L12; L49; L27; |
| | | L11; L52 | | L59; L72; L11 |
| 30 | SVIYLYLTFY | L12; L11; L14; L6; L7; | SNFGAISSV | L3; L28; L69; L20; L51; L4; L70; |
| | | L10; L58 | | L64; L58; L73 |
| 31 | SVKGLQPSV | L13; L51; L58; L20; L39; | SSFKWDLTAF | L53; L16; L10; L43; L26; L27; L58; |
| | | L3; L5 | | L59; L52; L54 |
| 32 | SVRVVTTF | L26; L43; L58; L29; L23; | SSRLSFKEL | L59; L60; L13; L73; L39; L57; L58; |
| | | L52; L59 | | L27; L61; L68 |
| 33 | SVTTEILPV | L20; L24; L13; L3; L7; L4; | STAALGVLM | L58; L20; L19; L7; L53; L21; L12; |
| | | L11 | | L52; L11; L70 |
| 34 | SVYAWNRKR | L7; L21; L15; L17; L19; | STFEEAALC | L7; L19; L14; L20; L13; L53; L11; |
| | | L6; L11 | | L12; L21; L52 |
| 35 | SWMESEFRVY | L27; L14; L10; L26; L40; | STKHFYWFF | L58; L43; L53; L13; L10; L11; L52; |
| | | L41; L29 | | L12; L70; L69 |
| 36 | SWVMRIMTW | L49; L10; L69; L8; L54; | SVFNICQAV | L24; L20; L13; L3; L64; L58; L4; |
| | | L9; L25 | | L69; L5; L28 |
| 37 | SYFAVHFI | L48; L71; L72; L64; L9; | SYIVDSVTV | L72; L71; L28; L13; L9; L8; L38; |
| | | L47; L62 | | L62; L35; L45 |
| 38 | SYFVVKRHTF | L8; L71; L72; L9; L16; | TASWFTAL | L67; L59; L61; L60; L57; L74; L68; |
| | | L12; L52 | | L23; L70; L32 |
| 39 | TEIDPKLDN | L46; L41; L36; L45; L38; | TEVVGDIIL | L36; L37; L29; L41; L38; L40; L45; |
| | | L37; L40 | | L32; L25; L34 |
| 40 | TEMLAKAL | L33; L29; L25; L37; L45; | TFFKLVNKF | L8; L9; L29; L72; L12; L62; L49; |
| | | L23; L40 | | L71; L16; L34 |
| 41 | TETDLTKGP | L46; L38; L42; L41; L40; | TGYHFREL | L23; L59; L65; L25; L48; L61; L55; |
| | | L45; L37 | | L47; L64; L66 |
| 42 | TEVLTEEVV | L45; L36; L37; L38; L29; | TPFEIKLAK | L31; L50; L49; L19; L7; L6; L32; |
| | | L46; L42 | | L51; L56; L13 |
| 43 | TGVEHVTFF | L69; L8; L65; L33; L9; | TPKGPKVKY | L31; L49; L39; L26; L51; L65; L14; |
| | | L29; L49 | | L56; L13; L12 |
| 44 | THLSVDTKF | L34; L8; L35; L28; L27; | TRFQTLLAL | L35; L25; L65; L64; L30; L34; L66; |
| | | L9; L12 | | L28; L4; L3 |
| 45 | TIKGTHHWL | L23; L39; L58; L67; L60; | TSVVLLSVL | L69; L73; L70; L53; L60; L61; L67; |
| | | L10; L74 | | L59; L55; L64 |
| 46 | TIQITISSF | L26; L10; L28; L43; L16; | TTIKPVTY | L54; L55; L58; L26; L70; L52; L29; |
| | | L72; L39 | | L69; L53; L43 |
| 47 | TLLALHRSY | L12; L14; L16; L27; L26; | TTKGGRFVL | L70; L65; L16; L53; L39; L55; L69; |
| | | L10; L55 | | L10; L20; L58 |
| 48 | TLPVNVAF | L57; L62; L26; L71; L72; | TVEEAKTVL | L63; L68; L32; L39; L23; L31; L62; |
| | | L43; L23 | | L56; L67; L34 |
| 49 | TNGDFLHFL | L65; L73; L4; L64; L20; | TVNVLAWLY | L12; L14; L18; L1; L31; L26; L52; |
| | | L34; L69 | | L55; L6; L16 |
| 50 | TNLPLQLGF | L12; L8; L52; L41; L9; | TVYDDGARR | L19; L17; L15; L21; L11; L6; L7; |
| | | L40; L16 | | L20; L14; L10 |
| 51 | TPAFDKSAF | L31; L49; L22; L32; L39; | TVYDPLQPEL | L67; L20; L60; L28; L61; L5; L2; |
| | | L56; L23 | | L4; L6; L13 |
| 52 | TPGSGVPVV | L47; L51; L39; L32; L50; | VAAGLEAPF | L58; L31; L74; L70; L43; L49; L12; |
| | | L22; L56 | | L59; L52; L53 |
| 53 | TQLGIEFLKR | L15; L7; L17; L21; L30; | VAKNLNESL | L43; L67; L70; L58; L69; L53; L68; |
| | | L19; L6 | | L60; L61; L56 |
| 54 | TQSRNLQEF | L26; L34; L58; L24; L27; | VCINGLMLL | L8; L34; L9; L74; L61; L69; L73; |
| | | L8; L35 | | L60; L4; L59 |
| 55 | TRFASVYAW | L35; L25; L65; L30; L34; | VGYLQPRTF | L55; L27; L47; L26; L52; L69; L9; |
| | | L66; L16 | | L48; L59; L65 |
| 56 | TRFFYVLGL | L65; L35; L30; L25; L64; | VLDWLEEKF | L5; L62; L63; L68; L1; L9; L12; |
| | | L66; L34 | | L49; L74; L18 |
| 57 | TSFGPLVRK | L7; L6; L19; L13; L20; | VLLPLTQY | L26; L59; L27; L43; L6; L14; L12; |
| | | L17; L15 | | L1; L4; L18 |
| 58 | TSKTTVASL | L65; L70; L23; L57; L10; | VLYENQKLI | L48; L3; L2; L24; L5; L16; L55; |
| | | L58; L39 | | L64; L4; L9 |
| 59 | TSLSGFKLK | L7; L13; L19; L17; L6; | VPWDTIANY | L49; L31; L43; L14; L12; L65; L51; |
| | | L15; L55 | | L47; L18; L27 |
| 60 | TTAAKLMVV | L20; L73; L70; L69; L3; | VSDADSTL | L63; L68; L62; L60; L1; L67; L74; |
| | | L10; L58 | | L55; L61; L54 |
| 61 | TTADIVVED | L20; L19; L64; L69; L7; | VSDIDYVPL | L68; L63; L67; L1; L60; L74; L62; |
| | | L17; L65 | | L53; L43; L61 |
| 62 | TTIKPVTYK | L13; L7; L19; L6; L17; | VTANVNALL | L20; L53; L70; L73; L54; L58; L60; |
| | | L20; L15 | | L1; L52; L61 |
| 63 | TTYPGQGLNGY | L11; L14; L1; L18; L6; | VTVYSHLLL | L73; L60; L61; L67; L70; L53; L1; |
| | | L16; L10 | | L8; L20; L69 |
| 64 | TVATSRTLSY | L10; L18; L11; L12; L6; | VVAIDYKHY | L26; L14; L11; L10; L18; L52; L43; |
| | | L14; L26 | | L12; L1; L58 |
| 65 | TVIEVQGYK | L7; L13; L6; L19; L20; | VVDSYYSLL | L63; L68; L5; L74; L1; L62; L67; |
| | | L14; L11 | | L18; L60; L58 |
| 66 | TVKPGNFNK | L7; L13; L19; L17; L15; | VVFLHVTY | L26; L29; L59; L27; L12; L52; L18; |
| | | L6; L21 | | L54; L31; L58 |
| 67 | TYTGAIKL | L62; L72; L66; L71; L8; | WLIVGVALL | L74; L3; L2; L4; L5; L20; L16; L34; |
| | | L64; L9 | | L62; L28 |
| 68 | VAIDYKHY | L43; L58; L70; L59; L69; | WLMWLIINL | L4; L2; L5; L74; L3; L64; L13; L65; |
| | | L14; L26 | | L20; L69 |
| 69 | VANGDSEVV | L58; L70; L49; L32; L68; | WTAGAAAYY | L14; L11; L58; L1; L18; L12; L10; |
| | | L43; L67 | | L54; L70; L52 |
| 70 | VAVPTGYVD | L49; L61; L67; L32; L60; | YANRNRFL | L60; L61; L70; L58; L74; L59; L64; |
| | | L70; L54 | | L63; L68; L55 |
| 71 | VELGTEVNEF | L41; L40; L8; L36; L9; | YANRNRFLY | L58; L12; L1; L59; L70; L49; L69; |
| | | L29; L37 | | L31; L18; L14 |
| 72 | VENPDILRVY | L40; L41; L29; L14; L26; | YCNKTVGEL | L65; L32; L58; L74; L57; L66; L62; |
| | | L52; L27 | | L61; L71; L72 |
| 73 | VEVQPQLEM | L36; L29; L45; L37; L46; | YENFNQHEV | L45; L37; L38; L42; L46; L29; L48; |
| | | L38; L41 | | L33; L36; L73 |
| 74 | VFLFVAAIF | L72; L62; L9; L12; L8; | YGDFSHSQL | L68; L63; L62; L28; L60; L67; L61; |
| | | L71; L31 | | L34; L57; L1 |
| 75 | VFNGVSFSTF | L8; L9; L62; L72; L71; | YLNSTNVTI | L2; L4; L24; L5; L3; L34; L49; L16; |
| | | L26; L12 | | L44; L8 |
| 76 | VINGDRWFL | L58; L60; L16; L73; L74; | YLTNDVSFL | L74; L4; L2; L3; L5; L64; L57; L61; |
| | | L4; L63 | | L62; L44 |
| 77 | VIPDYNTY | L43; L66; L26; L57; L59; | YMPYFFTLL | L57; L5; L64; L67; L62; L9; L66; |
| | | L72; L71 | | L8; L59; L74 |
| 78 | VKNGSIHLY | L14; L27; L66; L18; L12; | YPLECIKDL | L32; L39; L47; L31; L56; L23; L22; |
| | | L26; L58 | | L25; L50; L49 |
| 79 | VLSEARQHL | L4; L2; L5; L57; L16; L3; | YSDVENPHL | L68; L63; L60; L1; L74; L61; L62; |
| | | L9 | | L34; L5; L54 |
| 80 | VLTEEVVLK | L7; L6; L13; L2; L19; L4; | YSKHTPINL | L74; L58; L70; L60; L69; L43; L61; |
| | | L21 | | L67; L73; L64 |
| 81 | VLVPHVGEI | L3; L5; L4; L2; L74; L49; | YTNDKACPL | L61; L73; L60; L70; L43; L58; L59; |
| | | L69 | | L72; L57; L71 |
| 82 | VLYNSASF | L43; L26; L27; L72; L59; | YVRNLQHRL | L58; L20; L4; L10; L67; L11; L60; |
| | | L71; L62 | | L5; L44; L39 |
| 83 | VPANSTVL | L22; L56; L39; L47; L25; | AASGNLLL | L74; L70; L67; L61; L73; L69; L60; |
| | | L51; L31 | | L68; L58; L64; L63 |
| 84 | VPHISRQRL | L56; L39; L22; L32; L49; | AEAELAKNV | L45; L46; L42; L38; L37; L40; L41; |
| | | L31; L25 | | L36; L33; L29; L24 |
| 85 | VSLDNVLSTF | L53; L52; L54; L8; L16; | AIMQLFFSY | L12; L26; L7; L6; L14; L16; L18; |
| | | L9; L59 | | L10; L40; L27; L1 |
| 86 | VTDVTQLY | L18; L1; L54; L62; L63; | ALYNKYKYF | L43; L10; L16; L26; L69; L27; L12; |
| | | L52; L68 | | L11; L9; L3; L6 |
| 87 | VTFQSAVKR | L19; L7; L15; L21; L17; | ANGDSEVVL | L44; L67; L65; L35; L73; L66; L34; |
| | | L6; L52 | | L28; L36; L69; L56 |
| 88 | VTIAEILL | L74; L73; L70; L60; L53; | AQNSVRVL | L65; L44; L48; L27; L64; L35; L55; |
| | | L67; L55 | | L26; L73; L24; L69 |
| 89 | VVDDPCPIHFY | L18; L14; L1; L12; L11; | ASFDNFKFV | L73; L70; L69; L13; L45; L58; L48; |
| | | L5; L6 | | L64; L20; L3; L24 |
| 90 | VVFVLWAHGF | L16; L12; L26; L52; L53; | ASLPFGWLI | L55; L53; L52; L73; L48; L54; L24; |
| | | L11; L31 | | L7; L8; L16; L69 |
| 91 | VVIGTSKF | L58; L26; L74; L11; L54; | ATLPKGIMM | L73; L7; L13; L6; L53; L70; L52; |
| | | L63; L60 | | L61; L60; L58; L69 |
| 92 | VVRIKIVQM | L13; L26; L11; L58; L43; | AVINGDRWF | L10; L11; L26; L52; L55; L16; L58; |
| | | L39; L23 | | L27; L14; L43; L53 |
| 93 | VYEKLKPVL | L9; L8; L66; L62; L72; | DLNETLVTM | L11; L4; L23; L2; L5; L49; L3; L10; |
| | | L71; L63 | | L62; L32; L26 |
| 94 | VYYTSNPTTF | L8; L9; L71; L72; L62; | EPVLKGVKL | L23; L56; L39; L32; L22; L49; L31; |
| | | L12; L66 | | L50; L25; L20; L11 |
| 95 | WADNNCYL | L68; L63; L74; L67; L62; | ETTADIVVF | L11; L10; L53; L20; L52; L54; L31; |
| | | L60; L70 | | L49; L9; L19; L70 |
| 96 | WKYPQVNGL | L27; L44; L64; L25; L28; | FACPDGVKH | L59; L43; L58; L49; L31; L67; L32; |
| | | L66; L3 | | L69; L70; L61; L68 |
| 97 | WLDDVVYC | L62; L5; L63; L68; L74; | FAIGLALY | L59; L58; L69; L70; L12; L31; L14; |
| | | L1; L2 | | L74; L43; L49; L1 |
| 98 | WLIVGVAL | L23; L61; L59; L71; L43; | FAYTKRNVI | L70; L69; L47; L59; L61; L67; L58; |
| | | L72; L74 | | L64; L32; L49; L68 |
| 99 | WLPTGTLLV | L5; L24; L2; L57; L64; L3; | FFSYFAVHF | L62; L72; L71; L12; L9; L59; L58; |
| | | L4 | | L66; L69; L8; L31 |
| 100 | YEKLKPVL | L23; L37; L29; L33; L45; | FLAFVVFL | L74; L2; L62; L64; L3; L4; L68; |
| | | L36; L25 | | L23; L69; L61; L20 |
| 101 | YENQKLIAN | L46; L29; L37; L45; L38; | FLWLLWPVTL | L2; L5; L62; L3; L67; L68; L4; L74; |
| | | L42; L33 | | L44; L57; L69 |
| 102 | YFLQSINF | L71; L72; L9; L8; L62; | FLYENAFL | L74; L2; L62; L61; L68; L59; L5; |
| | | L12; L33 | | L3; L4; L57; L63 |
| 103 | YFMRFRRAF | L71; L72; L59; L9; L12; | FNPETNILL | L64; L57; L5; L62; L66; L67; L74; |
| | | L8; L62 | | L68; L63; L71; L61 |
| 104 | YFRLTLGVY | L72; L27; L14; L71; L12; | FSLWVYKQF | L60; L59; L53; L52; L55; L58; L43; |
| | | L26; L59 | | L61; L70; L69; L10 |
| 105 | YFYTSKTTV | L71; L72; L45; L62; L48; | FSYFA VHFI | L70; L69; L73; L47; L64; L55; L61; |
| | | L46; L47 | | L68; L74; L67; L20 |
| 106 | YGADLKSF | L59; L43; L23; L27; L26; | FVFKNIDGY | L58; L26; L11; L10; L12; L16; L14; |
| | | L69; L66 | | L49; L31; L1; L18 |
| 107 | YHTTDPSFL | L28; L34; L74; L35; L44; | GGAPTKVTF | L27; L26; L44; L65; L54; L49; L52; |
| | | L60; L61 | | L34; L53; L9; L8 |
| 108 | YIKWPWYIWL | L43; L60; L13; L58; L39; | GLFCLLNRY | L12; L26; L6; L14; L27; L18; L16; |
| | | L67; L68 | | L4; L10; L11; L3 |
| 109 | YLDAYNMMI | L5; L68; L1; L2; L34; L24; | GVVFLHVTY | L26; L12; L27; L7; L52; L14; L49; |
| | | L63 | | L13; L31; L18; L10 |
| 110 | YLEGSVRVV | L3; L2; L64; L24; L5; L63; | GYKSVNITF | L8; L9; L66; L13; L26; L27; L12; |
| | | L4 | | L72; L53; L71; L52 |
| 111 | YMPYFFTL | L57; L66; L5; L59; L67; | HLKDGTCGL | L43; L3; L11; L10; L2; L26; L13; |
| | | L47; L71 | | L23; L4; L44; L16 |
| 112 | YPVASPNEC | L49; L32; L31; L50; L51; | IAIVMVTIM | L70; L69; L61; L59; L32; L67; L74; |
| | | L47; L22 | | L31; L60; L58; L47 |
| 113 | YTNSFTRGV | L73; L58; L64; L70; L11; | IANYAKPFL | L61; L60; L58; L63; L59; L74; L68; |
| | | L48; L3 | | L70; L73; L31; L47 |
| 114 | YWFFSNYL | L62; L71; L72; L60; L61; | IFFITGNTL | L72; L71; L8; L9; L62; L28; L48; |
| | | L74; L59 | | L34; L25; L61; L67 |
| 115 | YYKKDNSYF | L8; L9; L58; L72; L71; | IIWFLLLSV | L5; L4; L24; L73; L3; L50; L2; L47; |
| | | L66; L12 | | L67; L18; L69 |
| 116 | YYPDKVFRS | L66; L8; L12; L9; L72; | IKWPWYIWL | L44; L66; L27; L64; L74; L25; L61; |
| | | L64; L71 | | L67; L65; L69; L60 |
| 117 | AAAYYVGYL | L74; L69; L67; L70; L58; | ISTKHFYWF | L53; L54; L55; L8; L52; L59; L73; |
| | | L61; L64; L60 | | L74; L9; L58; L60 |
| 118 | AALQIPFAM | L61; L31; L67; L12; L32; | ITILDGISQY | L11; L18; L10; L14; L52; L1; L26; |
| | | L60; L70; L69 | | L53; L12; L16; L27 |
| 119 | AAYYVGYL | L74; L67; L69; L70; L61; | IYNDKVAGF | L72; L9; L8; L62; L71; L66; L12; |
| | | L58; L59; L60 | | L26; L11; L43; L58 |
| 120 | AEILLIIM | L29; L37; L33; L45; L36; | KACPLIAAV | L69; L70; L73; L3; L67; L58; L5; |
| | | L42; L46; L38 | | L51; L64; L20; L50 |
| 121 | AEIPKEEVK | L36; L37; L42; L38; L46; | KENSYTTTI | L45; L37; L36; L38; L48; L24; L46; |
| | | L45; L40; L41 | | L41; L8; L42; L33 |
| 122 | AEIPKEEVKP | L46; L38; L42; L36; L37; | KFTDGVCLF | L8; L9; L54; L62; L72; L16; L12; |
| | | L40; L41; L45 | | L71; L52; L53; L14 |
| 123 | AHFPREGVF | L27; L28; L34; L35; L33; | KIEELFYSY | L18; L14; L16; L12; L1; L26; L13; |
| | | L69; L26; L44 | | L54; L27; L5; L6 |
| 124 | AIVVTCLAY | L43; L12; L26; L18; L6; | KLKPVLDWL | L3; L5; L4; L16; L2; L13; L74; L26; |
| | | L14; L59; L16 | | L14; L53; L39 |
| 125 | ALEPLVDL | L62; L23; L63; L74; L68; | KMFDAYVNTF | L16; L53; L26; L54; L9; L52; L8; |
| | | L4; L5; L1 | | L27; L3; L72; L71 |
| 126 | ALLSDLQDL | L4; L2; L24; L5; L3; L16; | LADKFPVL | L68; L63; L67; L74; L61; L60; L47; |
| | | L44; L74 | | L59; L32; L70; L62 |
| 127 | AQKFNGLTV | L24; L48; L26; L3; L58; | LIISVTSNY | L26; L43; L58; L14; L11; L12; L6; |
| | | L13; L27; L45 | | L18; L10; L1; L59 |
| 128 | AQTGIAVL | L44; L35; L27; L28; L24; | LLLDDFVEI | L2; L5; L4; L24; L3; L8; L16; L9; |
| | | L26; L25; L48 | | L67; L47; L49 |
| 129 | ASIKNFKSV | L73; L48; L46; L70; L69; | LPPAYTNSF | L31; L39; L49; L22; L47; L32; L56; |
| | | L58; L45; L13 | | L72; L71; L57; L9 |
| 130 | ASQSIIAY | L18; L43; L26; L1; L70; | LQTPFEIKL | L44; L64; L69; L74; L28; L24; L58; |
| | | L14; L27; L59 | | L73; L26; L35; L34 |
| 131 | AVHECFVKR | L15; L21; L7; L19; L17; | LRSDVLLPL | L66; L64; L30; L35; L34; L65; L67; |
| | | L6; L13; L11 | | L59; L25; L61; L4 |
| 132 | AVITREVGF | L26; L10; L11; L58; L52; | LTKHPNQEY | L43; L58; L26; L13; L70; L12; L1; |
| | | L12; L54; L16 | | L52; L14; L53; L18 |
| 133 | AYKIEELFY | L12; L14; L72; L13; L65; | LVRDLPQGF | L58; L52; L26; L43; L12; L16; L49; |
| | | L71; L66; L26 | | L11; L53; L10; L31 |
| 134 | CANDPVGF | L58; L59; L43; L62; L70; | MSAPPAQY | L70; L54; L1; L18; L59; L43; L69; |
| | | L54; L49; L69 | | L14; L58; L26; L55 |
| 135 | CEIVGGQIV | L45; L37; L38; L36; L42; | NATNVVIKV | L20; L70; L69; L64; L47; L58; L24; |
| | | L46; L29; L24 | | L73; L49; L50; L19 |
| 136 | CESHGKQVV | L37; L45; L42; L38; L24; | NSIAIPTNF | L58; L54; L53; L52; L70; L9; L74; |
| | | L46; L36; L29 | | L69; L60; L55; L49 |
| 137 | CPDGVKHVY | L31; L49; L32; L65; L14; | QPTESIVRF | L49; L31; L56; L39; L32; L22; L34; |
| | | L18; L39; L1 | | L11; L50; L9; L29 |
| 138 | DEFIERYKL | L29; L37; L25; L36; L41; | RAMPNMLRI | L73; L69; L24; L54; L47; L70; L52; |
| | | L40; L45; L33 | | L53; L55; L8; L49 |
| 139 | DEFTPFDVV | L29; L45; L37; L46; L47; | REHEHEIAW | L38; L33; L46; L42; L37; L41; L40; |
| | | L25; L36; L42 | | L29; L36; L45; L54 |
| 140 | DLNGDVVAI | L10; L49; L3; L4; L2; L23; | REHEHEIAWY | L14; L29; L41; L40; L38; L27; L46; |
| | | L24; L11 | | L26; L36; L42; L43 |
| 141 | DVFYKENSY | L11; L10; L29; L16; L12; | REVLSDREL | L36; L37; L45; L40; L41; L38; L33; |
| | | L26; L14; L19 | | L55; L27; L65; L29 |
| 142 | DYQGKPLEF | L8; L9; L72; L71; L66; | RSFIEDLLF | L53; L54; L52; L55; L16; L73; L18; |
| | | L12; L10; L62 | | L65; L27; L26; L9 |
| 143 | EAARYMRSL | L10; L32; L60; L70; L61; | RTNVYLAVF | L16; L53; L54; L52; L73; L65; L58; |
| | | L20; L11; L69 | | L26; L18; L14; L59 |
| 144 | EKFKEGVEF | L27; L44; L34; L11; L10; | RYFKYWDQT | L16; L14; L26; L71; L65; L8; L27; |
| | | L49; L26; L8 | Y | L54; L72; L12; L66 |
| 145 | EMLDNRATL | L8; L16; L25; L4; L10; | SALWEIQQV | L24; L4; L47; L69; L70; L32; L73; |
| | | L34; L40; L41 | | L64; L58; L2; L13 |
| 146 | ENPDILRVY | L11; L10; L40; L41; L64; | SANNCTFEY | L58; L43; L59; L49; L70; L12; L14; |
| | | L14; L66; L26 | | L69; L26; L31; L27 |
| 147 | EQFKKGVQI | L24; L48; L44; L34; L35; | SEAVEAPLV | L38; L45; L42; L36; L37; L40; L46; |
| | | L20; L27; L25 | | L28; L41; L24; L29 |
| 148 | ETIQITISSF | L11; L10; L20; L52; L19; | SEDNQTTTI | L45; L37; L34; L36; L42; L41; L68; |
| | | L53; L41; L40 | | L38; L40; L48; L24 |
| 149 | EVVGDIILK | L19; L7; L20; L11; L17; | SEFDRDAAM | L29; L46; L36; L37; L33; L40; L38; |
| | | L6; L10; L13 | | L45; L25; L42; L41 |
| 150 | EWFLAYILF | L9; L41; L40; L8; L34; | SEIIGYKAI | L45; L40; L41; L37; L48; L36; L42; |
| | | L62; L29; L31 | | L46; L38; L29; L25 |
| 151 | EYADVFHL | L9; L8; L66; L20; L72; | SFNPETNIL | L62; L72; L71; L28; L8; L9; L34; |
| | | L71; L65; L35 | | L32; L65; L64; L35 |
| 152 | EYFNSVCRL | L8; L9; L72; L71; L20; | SHKPPISF | L43; L28; L23; L27; L66; L58; L35; |
| | | L34; L64; L10 | | L26; L39; L29; L70 |
| 153 | FAAETLKAT | L59; L43; L61; L69; L60; | SSSDNIAL | L67; L61; L70; L68; L60; L56; L59; |
| | | L58; L70; L32 | | L35; L57; L73; L74 |
| 154 | FAFPFTIYSLL | L67; L61; L31; L74; L60; | SVLNDILSR | L7; L15; L17; L6; L19; L21; L11; |
| | | L69; L58; L70 | | L16; L13; L4; L10 |
| 155 | FEIKLAKKF | L29; L41; L40; L37; L45; | SWNADLYKL | L4; L8; L34; L62; L56; L9; L32; |
| | | L58; L36; L49 | | L35; L37; L30; L39 |
| 156 | FELEDFIPM | L29; L46; L45; L36; L42; | SYSGQSTQL | L9; L72; L8; L66; L71; L28; L65; |
| | | L40; L31; L37 | | L35; L64; L62; L56 |
| 157 | FLAHIQWMV | L2; L20; L4; L24; L5; L3; | TELEPPCRF | L29; L41; L40; L33; L36; L45; L37; |
| | | L69; L64 | | L8; L9; L38; L46 |
| 158 | FLAYILFTR | L19; L21; L17; L15; L4; | TLIVNSVLL | L74; L2; L4; L28; L62; L34; L26; |
| | | L2; L3; L20 | | L9; L8; L5; L44 |
| 159 | FNPETNIL | L57; L66; L62; L67; L71; | TPFDVVRQC | L32; L31; L49; L51; L39; L47; L64; |
| | | L72; L59; L64 | | L65; L19; L56; L50 |
| 160 | FPDLNGDVV | L50; L32; L47; L68; L22; | TPSGTWLTY | L49; L31; L65; L32; L26; L12; L18; |
| | | L62; L39; L31 | | L22; L51; L39; L14 |
| 161 | FQPTNGVGY | L26; L27; L58; L71; L12; | VASKILGL | L74; L70; L61; L60; L58; L59; L23; |
| | | L11; L72; L66 | | L73; L67; L56; L47 |
| 162 | FRYMNSQGL | L25; L64; L35; L30; L66; | VDTDFVNEF | L9; L8; L59; L66; L43; L29; L58; |
| | | L28; L34; L61 | | L49; L72; L69; L26 |
| 163 | FSASTSAF | L59; L58; L43; L60; L70; | VIYLYLTFY | L6; L12; L43; L58; L26; L59; L1; |
| | | L61; L74; L54 | | L18; L14; L69; L27 |
| 164 | FTDGVCLF | L63; L62; L1; L58; L68; | VLYENQKL | L2; L24; L74; L4; L25; L5; L3; L55; |
| | | L70; L74; L60 | | L68; L63; L64 |
| 165 | FVAAIFYLI | L20; L12; L47; L5; L49; | VMFTPLVPF | L9; L43; L26; L59; L6; L16; L71; |
| | | L64; L74; L24 | | L72; L52; L31; L12 |
| 166 | FVLALLSDL | L74; L60; L4; L67; L61; | VSDIDITF | L63; L68; L54; L1; L53; L62; L18; |
| | | L59; L31; L5 | | L74; L52; L55; L60 |
| 167 | FVSLAIDAY | L31; L1; L59; L14; L12; | VTIDYTEISF | L53; L52; L16; L10; L43; L11; L8; |
| | | L26; L58; L18 | | L58; L12; L26; L9 |
| 168 | FYEDFLEY | L12; L59; L72; L71; L66; | VVDDPCPIHF | L5; L63; L68; L62; L60; L12; L74; |
| | | L1; L62; L18 | | L58; L16; L52; L9 |
| 169 | FYLTNDVSF | L71; L72; L9; L8; L66; | VVDYGARFY | L18; L1; L14; L55; L12; L63; L58; |
| | | L12; L62; L52 | | L68; L52; L62; L49 |
| 170 | GEAANFCAL | L37; L36; L46; L45; L40; | VVNQNAQAL | L60; L57; L39; L61; L56; L32; L68; |
| | | L41; L44; L38 | | L58; L16; L59; L26 |
| 171 | GEIPVAYRK | L37; L36; L45; L7; L41; | VVVNAANVY | L26; L12; L14; L31; L58; L52; L10; |
| | | L40; L38; L29 | | L16; L54; L69; L59 |
| 172 | GEIPVAYRKV | L45; L37; L38; L46; L36; | YANSVFNIC | L59; L60; L61; L32; L58; L70; L49; |
| | | L41; L40; L24 | | L69; L67; L31; L47 |
| 173 | GELGDVRET | L37; L45; L46; L41; L40; | YFTEQPIDL | L62; L72; L74; L71; L9; L8; L64; |
| | | L36; L38; L42 | | L61; L67; L60; L66 |
| 174 | GEVFNATRF | L37; L40; L41; L29; L45; | YFVVKRHTF | L71; L72; L8; L9; L49; L62; L12; |
| | | L36; L27; L33 | | L43; L59; L29; L60 |
| 175 | GIITTVAAF | L10; L26; L11; L58; L27; | YIVDSVTV | L58; L28; L43; L48; L61; L20; L2; |
| | | L28; L12; L43 | | L59; L73; L67; L69 |
| 176 | GNFKNLREF | L27; L26; L10; L52; L55; | YKKPASREL | L66; L27; L60; L64; L44; L61; L58; |
| | | L37; L49; L58 | | L59; L43; L39; L70 |
| 177 | GVYSVIYLYL | L6; L74; L67; L10; L13; | YSFLPGVY | L1; L59; L70; L69; L55; L27; L58; |
| | | L7; L20; L11 | | L26; L43; L14; L66 |
| 178 | GWIFGTTL | L28; L71; L37; L72; L60; | YSVIYLYL | L61; L74; L60; L73; L67; L59; L65; |
| | | L48; L61; L35 | | L69; L1; L70; L55 |
| 179 | GYAFEHIVY | L12; L66; L27; L72; L26; | YYTSNPTTF | L9; L8; L66; L72; L71; L62; L58; |
| | | L71; L14; L65 | | L12; L49; L60; L27 |
| 180 | HEFCSQHTM | L29; L37; L36; L45; L46; | AANFCALIL | L73; L61; L69; L70; L74; L59; L67; |
| | | L32; L33; L38 | | L60; L32; L68; L31; L58 |
| 181 | HFAIGLALY | L12; L14; L18; L11; L72; | ACFVLAAVY | L14; L27; L12; L26; L40; L31; L69; |
| | | L71; L26; L49 | | L41; L72; L18; L71; L16 |
| 182 | HSYFTSDYY | L14; L18; L1; L65; L54; | AELEGIQY | L29; L41; L40; L27; L45; L33; L37; |
| | | L43; L58; L12 | | L42; L46; L26; L38; L36 |
| 183 | HTTDPSFL | L73; L60; L20; L70; L62; | ALCEKALKY | L12; L18; L6; L14; L16; L26; L1; |
| | | L55; L61; L63 | | L11; L43; L10; L13; L27 |
| 184 | HVQLSLPVL | L28; L32; L39; L59; L56; | ALLTKSSEY | L26; L12; L14; L27; L16; L18; L6; |
| | | L31; L67; L60 | | L43; L10; L72; L71; L1 |
| 185 | IADYNYKL | L63; L68; L67; L62; L74; | ALWEIQQVV | L5; L24; L2; L4; L3; L64; L13; L16; |
| | | L60; L54; L70 | | L69; L46; L44; L37 |
| 186 | IAEILLIIM | L31; L32; L69; L63; L67; | ATSRTLSYY | L1; L14; L18; L58; L6; L12; L54; |
| | | L68; L70; L47 | | L52; L11; L53; L7; L10 |
| 187 | IIIWFLLL | L74; L73; L60; L61; L67; | AVVCFNSTY | L26; L14; L11; L10; L16; L43; L12; |
| | | L59; L47; L48 | | L18; L58; L54; L27; L52 |
| 188 | ILFTRFFYV | L5; L4; L3; L2; L6; L69; | AYILFTRFF | L9; L72; L71; L8; L40; L64; L62; |
| | | L73; L20 | | L12; L27; L41; L66; L26 |
| 189 | ILTSLLVLV | L2; L4; L5; L3; L24; L64; | DEDDSEPVL | L36; L29; L35; L28; L37; L41; L34; |
| | | L73; L20 | | L32; L68; L25; L40; L63 |
| 190 | IPRRNVATL | L22; L56; L39; L32; L47; | EEHVQIHTI | L45; L37; L42; L41; L29; L48; L40; |
| | | L51; L31; L49 | | L24; L34; L36; L25; L46 |
| 191 | IQITISSF | L26; L27; L43; L58; L28; | EIKESVQTF | L10; L11; L26; L23; L43; L58; L16; |
| | | L48; L71; L72 | | L9; L39; L34; L8; L49 |
| 192 | ISDEFSSNV | L63; L68; L18; L1; L5; | ELTPVVQTI | L24; L20; L3; L10; L4; L49; L9; L8; |
| | | L62; L70; L73 | | L34; L5; L55; L2 |
| 193 | IVDTVSALVY | L18; L1; L12; L14; L68; | ELYSPIFLI | L24; L20; L11; L4; L17; L10; L16; |
| | | L52; L6; L54 | | L6; L2; L9; L8; L34 |
| 194 | IVVTCLAYY | L11; L12; L14; L58; L10; | FAYANRNRF | L58; L70; L69; L59; L60; L49; L74; |
| | | L18; L52; L6 | | L61; L47; L31; L12; L32 |
| 195 | KAALLADKF | L53; L54; L52; L58; L55; | FGDSVEEVL | L67; L68; L63; L62; L32; L34; L44; |
| | | L12; L31; L69 | | L61; L5; L31; L28; L74 |
| 196 | KATYKPNTW | L54; L52; L49; L53; L16; | FHLDGEVITF | L34; L35; L28; L72; L27; L71; L9; |
| | | L69; L55; L70 | | L31; L8; L60; L52; L12 |
| 197 | KAYKIEELFY | L12; L14; L18; L58; L54; | FPFTIYSL | L23; L32; L56; L47; L50; L31; L25; |
| | | L52; L27; L69 | | L66; L39; L22; L61; L49 |
| 198 | KFLTENLLL | L8; L9; L71; L72; L13; | FSYFA VHF | L59; L74; L58; L70; L60; L55; L69; |
| | | L53; L73; L16 | | L68; L62; L61; L43; L54 |
| 199 | KIILFLAL | L73; L59; L67; L60; L61; | FTEERLKLF | L1; L58; L60; L62; L10; L63; L69; |
| | | L23; L74; L68 | | L52; L70; L11; L59; L12 |
| 200 | KKFLPFQQF | L27; L16; L44; L33; L26; | FTNVYADSF | L53; L58; L59; L43; L71; L72; L62; |
| | | L9; L53; L52 | | L54; L52; L12; L60; L16 |
| 201 | KLDDKDPNF | L5; L63; L2; L16; L9; L54; | FVFPLNSI | L59; L47; L20; L64; L69; L48; L3; |
| | | L62; L18 | | L25; L67; L50; L68; L61 |
| 202 | KLFIRQEEV | L3; L2; L24; L5; L4; L16; | FVVSTGYHF | L58; L60; L11; L10; L12; L74; L49; |
| | | L23; L73 | | L54; L59; L31; L9; L52 |
| 203 | KLLHKPIVW | L16; L52; L6; L54; L4; L2; | GAVDINKL | L74; L70; L69; L60; L64; L61; L67; |
| | | L21; L53 | | L58; L44; L37; L59; L55 |
| 204 | KLMVVIPDY | L26; L14; L12; L6; L18; | GSVRVVTTF | L53; L54; L52; L26; L27; L55; L65; |
| | | L16; L43; L13 | | L10; L49; L58; L60; L74 |
| 205 | KLQDVVNQN | L4; L16; L2; L5; L14; L21; | GVVDYGARF | L10; L11; L12; L26; L49; L58; L54; |
| | | L3; L24 | | L52; L43; L14; L20; L27 |
| 206 | KNLSDRVVF | L16; L53; L52; L27; L54; | HAFHTPAF | L59; L49; L31; L58; L32; L70; L50; |
| | | L9; L8; L55 | | L60; L54; L43; L61; L47 |
| 207 | KQASLNGVTL | L44; L56; L27; L26; L16; | HVVAFNTLL | L20; L32; L56; L74; L31; L44; L64; |
| | | L24; L34; L13 | | L53; L34; L73; L11; L28 |
| 208 | KQFDTYNL | L44; L24; L16; L27; L26; | HWFVTQRNF | L49; L8; L9; L53; L52; L27; L65; |
| | | L33; L48; L65 | | L55; L62; L71; L34; L29 |
| 209 | KSAGFPFNK | L7; L6; L13; L54; L21; | IAAVITREV | L70; L64; L69; L58; L43; L47; L59; |
| | | L55; L19; L15 | | L49; L73; L61; L20; L32 |
| 210 | KSAPLIELC | L52; L53; L54; L55; L18; | IAIILASF | L59; L43; L54; L47; L58; L52; L60; |
| | | L73; L16; L69 | | L26; L53; L31; L23; L61 |
| 211 | KVAGFAKF | L54; L26; L58; L16; L53; | IAMACLVGL | L59; L31; L58; L60; L67; L56; L61; |
| | | L11; L52; L74 | | L69; L70; L74; L47; L63 |
| 212 | KVKPTVVV | L13; L51; L39; L23; L58; | IFTIGTVTL | L72; L62; L71; L9; L8; L28; L56; |
| | | L69; L73; L26 | | L61; L68; L49; L67; L34 |
| 213 | KVKPTVVVN | L13; L26; L52; L16; L51; | IFVDGVPFV | L71; L72; L64; L4; L8; L2; L62; |
| | | L69; L21; L39 | | L47; L20; L69; L9; L5 |
| 214 | KYLYFIKGL | L8; L9; L13; L71; L72; | ILKPANNSL | L3; L43; L23; L5; L26; L4; L68; |
| | | L66; L16; L65 | | L57; L2; L16; L39; L9 |
| 215 | KYPQVNGL | L66; L72; L71; L9; L62; | IQFPNTYL | L25; L28; L58; L26; L74; L44; L35; |
| | | L8; L57; L33 | | L48; L69; L27; L68; L64 |
| 216 | LAAECTIF | L59; L70; L69; L74; L58; | IRQEEVQEL | L66; L65; L64; L35; L34; L25; L28; |
| | | L31; L47; L60 | | L30; L72; L4; L71; L32 |
| 217 | LALGGSVAI | L47; L61; L32; L67; L50; | ITVNVLAWL | L20; L53; L60; L61; L73; L67; L59; |
| | | L49; L59; L31 | | L58; L10; L70; L69; L54 |
| 218 | LATHGLAAV | L50; L59; L61; L47; L32; | IVAIVVTCL | L74; L20; L67; L64; L60; L56; L26; |
| | | L70; L20; L58 | | L53; L61; L68; L39; L28 |
| 219 | LAVPYNMRV | L69; L70; L47; L58; L73; | KTIGPDMFL | L73; L60; L53; L61; L55; L54; L16; |
| | | L32; L50; L61 | | L58; L13; L20; L70; L69 |
| 220 | LCFLAFLLF | L31; L12; L9; L47; L59; | LAAIMQLFF | L59; L58; L31; L49; L52; L12; L47; |
| | | L41; L52; L40 | | L74; L60; L69; L53; L70 |
| 221 | LEMELTPVV | L38; L29; L45; L46; L42; | LAFVVFLL | L47; L74; L69; L70; L61; L60; L59; |
| | | L37; L36; L50 | | L31; L64; L55; L68; L23 |
| 222 | LEPEYFNSV | L45; L5; L57; L46; L38; | LEYHDVRVV | L45; L37; L46; L38; L47; L64; L42; |
| | | L37; L29; L42 | | L69; L29; L48; L70; L36 |
| 223 | LHFLPRVF | L28; L55; L27; L59; L29; | LIVNSVLLF | L12; L58; L60; L9; L74; L52; L8; |
| | | L35; L52; L47 | | L31; L26; L69; L49; L16 |
| 224 | LIDFYLCFL | L68; L5; L63; L60; L74; | LLDDFVEII | L5; L2; L68; L62; L63; L3; L1; L49; |
| | | L62; L1; L67 | | L24; L4; L67; L34 |
| 225 | LIKVTLVFL | L58; L23; L56; L39; L60; | LLFLAFVVF | L26; L59; L27; L31; L9; L16; L12; |
| | | L74; L59; L62 | | L62; L52; L69; L50; L5 |
| 226 | LINIIIWFL | L4; L5; L73; L60; L58; | LLLDRLNQL | L4; L2; L5; L23; L3; L59; L25; L8; |
| | | L61; L64; L74 | | L6; L16; L74; L64 |
| 227 | LPAPRTLL | L22; L47; L56; L23; L39; | LTNDVSFL | L62; L70; L60; L73; L55; L61; L63; |
| | | L31; L25; L32 | | L58; L59; L68; L64; L1 |
| 228 | LPFAMGIIAM | L31; L50; L32; L39; L22; | LVAELEGIQY | L12; L26; L18; L11; L6; L14; L31; |
| | | L56; L47; L51 | | L1; L52; L13; L43; L10 |
| 229 | LPIGINITR | L32; L31; L19; L50; L17; | LYIIKLIFL | L9; L72; L8; L71; L62; L64; L23; |
| | | L49; L56; L21 | | 74; L73; L60; L61; L69 |
| 230 | LPKEITVAT | L50; L51; L39; L56; L31; | MPYFFTLLL | L47; L31; L50; L22; L32; L49; L39; |
| | | L32; L43; L22 | | L73; L64; L69; L25; L56 |
| 231 | LPNDDTLRV | L49; L47; L50; L51; L32; | NALDQAISM | L70; L31; L61; L67; L32; L59; L69; |
| | | L39; L31; L4 | | L60; L71; L72; L47; L58 |
| 232 | LPYPDPSRI | L47; L49; L50; L32; L31; | NCYFPLQSY | L43; L26; L27; L31; L12; L59; L29; |
| | | L22; L39; L51 | | L14; L10; L11; L41; L40 |
| 233 | LPYPDPSRIL | L32; L39; L47; L22; L31; | NTNPIQLSSY | L11; L14; L10; L1; L43; L18; L16; |
| | | L56; L49; L51 | | L58; L12; L26; L6; L52 |
| 234 | LQIPFAMQM | L69; L58; L28; L26; L61; | NVFAFPFTI | L20; L69; L16; L47; L24; L55; L3; |
| | | L27; L59; L70 | | L64; L49; L34; L4; L12 |
| 235 | LRIMASLVL | L35; L25; L66; L61; L28; | PANNAAIVL | L61; L67; L70; L69; L32; L60; L58; |
| | | L34; L30; L64 | | L74; L68; L31; L59; L56 |
| 236 | LSDLQDLKW | L1; L54; L18; L52; L68; | PQIGVVREF | L26; L27; L58; L65; L43; L8; L64; |
| | | L63; L53; L49 | | L10; L34; L9; L69; L24 |
| 237 | LSYFIASF | L59; L43; L60; L52; L70; | RANSAVKL | L55; L73; L63; L54; L74; L56; L70; |
| | | L26; L55; L54 | | L58; L53; L44; L69; L39 |
| 238 | LSYGIATV | L48; L47; L70; L73; L69; | RARSVSPKL | L13; L58; L53; L44; L56; L54; L55; |
| | | L59; L43; L58 | | L39; L22; L70; L73; L63 |
| 239 | LVATAEAEL | L20; L56; L67; L61; L60; | RFKESPFEL | L56; L8; L72; L9; L62; L71; L13; |
| | | L28; L74; L32 | | L39; L55; L66; L16; L65 |
| 240 | LYLYALVYF | L9; L8; L62; L72; L71; | RGWIFGTTL | L44; L53; L55; L56; L27; L57; L65; |
| | | L12; L52; L31 | | L48; L26; L8; L16; L37 |
| 241 | LYYDSMSY | L71; L72; L66; L43; L12; | RITGLYPTL | L44; L56; L73; L16; L13; L39; L60; |
| | | L65; L59; L14 | | L54; L4; L5; L24; L61 |
| 242 | MADQAMTQM | L63; L68; L32; L49; L70; | RLYYDSMSY | L27; L26; L14; L43; L16; L6; L18; |
| | | L31; L60; L74 | | L65; L12; L13; L1; L54 |
| 243 | MLSDTLKNL | L4; L3; L2; L5; L64; L57; | RQEEVQEL | L24; L44; L65; L27; L63; L26; L33; |
| | | L74; L16 | | L68; L35; L48; L55; L25 |
| 244 | MPLSAPTL | L47; L50; L31; L22; L32; | SAKSASVYY | L58; L70; L43; L13; L69; L14; L12; |
| | | L56; L39; L25 | | L26; L49; L27; L52; L31 |
| 245 | MPYFFTLL | L47; L50; L22; L31; L23; | SAVVLLILM | L69; L58; L31; L70; L74; L64; L32; |
| | | L25; L74; L39 | | L52; L49; L59; L19; L60 |
| 246 | MSMTYGQQF | L53; L55; L54; L52; L43; | SLIYSTAAL | L3; L4; L57; L2; L28; L5; L72; L61; |
| | | L58; L70; L60 | | L43; L71; L59; L10 |
| 247 | MTYRRLISM | L69; L70; L25; L10; L11; | SLLSVLLSM | L4; L6; L2; L5; L12; L16; L3; L7; |
| | | L58; L59; L53 | | L59; L24; L11; L15 |
| 248 | MVSLLSVLL | L20; L74; L64; L31; L28; | SMWALIISV | L2; L4; L3; L5; L48; L13; L24; L69; |
| | | L56; L32; L67 | | L51; L64; L50; L20 |
| 249 | MYASAVVL | L71; L72; L66; L62; L35; | SQMEIDFLEL | L44; L67; L34; L73; L61; L27; L35; |
| | | L28; L9; L64 | | L8; L26; L4; L2; L5 |
| 250 | NARDGCVPL | L67; L43; L70; L61; L59; | STDVVYRAF | L62; L68; L43; L52; L53; L10; L60; |
| | | L69; L58; L60 | | L63; L1; L49; L58; L54 |
| 251 | NELSPVAL | L25; L29; L37; L36; L23; | SVASQSIIAY | L10; L26; L11; L6; L12; L7; L14; |
| | | L35; L33; L40 | | L43; L16; L18; L19; L27 |
| 252 | NLHSSRLSF | L16; L57; L12; L43; L27; | TEETFKLSY | L41; L29; L18; L46; L40; L42; L1; |
| | | L26; L72; L10 | | L38; L45; L65; L12; L36 |
| 253 | NLIDSYFVV | L5; L2; L3; L24; L4; L35; | TSNPTTFHL | L65; L73; L55; L60; L54; L8; L61; |
| | | L20; L11 | | L74; L70; L58; L68; L53 |
| 254 | NLWNTFTRL | L5; L4; L74; L2; L24; L57; | TTFTYASAL | L71; L20; L70; L10; L61; L72; L60; |
| | | L8; L3 | | L65; L57; L69; L28; L16 |
| 255 | NQTTTIQTI | L24; L48; L34; L35; L28; | TVAYFNMVY | L26; L11; L12; L14; L65; L31; L10; |
| | | L8; L25; L49 | | L18; L16; L58; L1; L69 |
| 256 | NSKVQIGEY | L43; L14; L26; L10; L70; | VAKHDFFKF | L58; L49; L53; L70; L52; L12; L43; |
| | | L1; L58; L18 | | L8; L9; L69; L31; L59 |
| 257 | NTLLFLMSF | L53; L52; L16; L59; L10; | VANGDSEVVL | L67; L32; L68; L31; L44; L56; L53; |
| | | L12; L11; L31 | | L63; L58; L70; L74; L60 |
| 258 | NVFAFPFTIY | L11; L6; L12; L10; L31; | VAYFNMVYM | L70; L69; L58; L59; L74; L61; L67; |
| | | L26; L29; L14 | | L60; L47; L43; L68; L63 |
| 259 | NVKDFMSL | L23; L39; L67; L10; L59; | VFLGIITTV | L48; L8; L72; L2; L71; L4; L9; L24; |
| | | L66; L25; L60 | | L47; L3; L62; L51 |
| 260 | NVPLHGTIL | L57; L67; L63; L39; L66; | VLNEKCSAY | L43; L26; L14; L16; L6; L12; L18; |
| | | L74; L11; L64 | | L10; L72; L27; L1; L57 |
| 261 | NWITGGIAI | L8; L35; L71; L34; L28; | VLWAHGFEL | L4; L2; L5; L57; L68; L61; L16; |
| | | L72; L9; L25 | | L59; L74; L39; L67; L63 |
| 262 | NYGDSATL | L66; L72; L71; L9; L62; | VRDPQTLEI | L35; L34; L68; L64; L65; L66; L62; |
| | | L8; L65; L35 | | L5; L63; L25; L49; L67 |
| 263 | NYLKRRVVF | L8; L71; L9; L72; L23; | VTLAILTAL | L4; L60; L73; L59; L67; L53; L61; |
| | | L66; L25; L27 | | L6; L20; L31; L16; L56 |
| 264 | PQADVEWKF | L44; L24; L26; L8; L9; | VVNAANVYL | L58; L68; L56; L73; L74; L62; L60; |
| | | L34; L58; L27 | | L63; L5; L4; L20; L61 |
| 265 | QIITTDNTF | L54; L9; L34; L26; L10; | WVYKQFDTY | L26; L10; L14; L12; L49; L11; L16; |
| | | L11; L8; L32 | | L27; L58; L18; L59; L6 |
| 266 | QLTPTWRVY | L26; L43; L14; L27; L16; | YFIASFRLF | L9; L8; L12; L72; L71; L62; L27; |
| | | L55; L65; L69 | | L49; L31; L60; L58; L34 |
| 267 | QVVDMSMTY | L11; L10; L26; L12; L43; | YFIKGLNNL | L8; L71; L72; L9; L74; L34; L62; |
| | | L14; L16; L58 | | L58; L67; L64; L3; L59 |
| 268 | REFVFKNID | L46; L37; L45; L36; L29; | YIDIGNYTV | L68; L5; L63; L2; L1; L35; L28; |
| | | L42; L38; L65 | | L24; L34; L18; L67; L32 |
| 269 | RELHLSWEV | L33; L45; L38; L24; L37; | YTTTIKPVTY | L52; L12; L58; L54; L69; L1; L10; |
| | | L42; L46; L36 | | L43; L70; L49; L18; L14 |
| 270 | REQIDGYVM | L36; L38; L37; L42; L29; | AEILLIIMR | L40; L41; L42; L37; L45; L36; L7; |
| | | L33; L45; L40 | | L38; L29; L21; L15; L46; L19 |
| 271 | RKSAPLIEL | L44; L56; L27; L73; L39; | APFLYLYAL | L56; L22; L39; L23; L31; L32; L50; |
| | | L66; L22; L64 | | L25; L44; L33; L51; L4; L67 |
| 272 | RLDKVEAEV | L5; L24; L2; L63; L18; | ASLNGVTL | L55; L61; L53; L67; L60; L54; L59; |
| | | L62; L68; L3 | | L44; L73; L56; L71; L74; L72 |
| 273 | RSGETLGVL | L65; L73; L53; L55; L54; | AYITGGVVQL | L9; L8; L71; L72; L56; L44; L13; |
| | | L44; L56; L14 | | L28; L37; L36; L34; L66; L4 |
| 274 | RSIFSRTL | L55; L73; L65; L60; L48; | CVEEVTTTL | L60; L68; L63; L5; L32; L67; L39; |
| | | L54; L61; L53 | | L61; L62; L44; L56; L20; L34 |
| 275 | RTIAFGGCVF | L54; L16; L53; L52; L55; | DTYNLWNTF | L10; L11; L53; L52; L49; L16; L54; |
| | | L26; L10; L8 | | L25; L29; L58; L70; L19; L69 |
| 276 | RVDGQVDL | L63; L68; L74; L62; L67; | DVFHLYLQY | L10; L11; L12; L49; L6; L14; L29; |
| | | L54; L56; L39 | | L31; L19; L16; L18; L26; L13 |
| 277 | RVYSTGSNV | L13; L24; L51; L58; L6; | FAPSASAFF | L58; L57; L59; L62; L63; L12; L61; |
| | | L14; L16; L3 | | L60; L74; L70; L9; L31; L72 |
| 278 | RYKLEGYAF | L9; L8; L71; L72; L26; | FIAGLIAIV | L3; L20; L58; L69; L2; L64; L4; |
| | | L66; L27; L14 | | L73; L67; L5; L11; L28; L24 |
| 279 | SAKNRARTV | L70; L58; L69; L13; L23; | FKEGSSVEL | L44; L27; L68; L66; L61; L62; L67; |
| | | L64; L43; L39 | | L63; L32; L60; L34; L35; L28 |
| 280 | SAKSASVY | L58; L70; L43; L69; L14; | FLFLTWICL | L74; L2; L69; L64; L4; L5; L62; |
| | | L59; L26; L27 | | L67; L3; L59; L61; L23; L68 |
| 281 | SEHDYQIGG | L42; L37; L46; L38; L36; | FLIVAAIVF | L59; L43; L26; L72; L74; L12; L62; |
| | | L41; L29; L40 | | L69; L71; L31; L61; L9; L68 |
| 282 | SELLTPLGI | L45; L41; L40; L36; L37; | FTDGVCLFW | L1; L68; L54; L18; L49; L63; L62; |
| | | L48; L42; L33 | | L60; L58; L67; L53; L52; L73 |
| 283 | SEYTGNYQC | L42; L45; L37; L46; L38; | HANEYRLY | L55; L49; L14; L43; L18; L59; L70; |
| | | L29; L36; L41 | | L65; L58; L54; L31; L64; L52 |
| 284 | SFGPLVRKI | L64; L8; L9; L48; L24; | HEGKTFYVL | L37; L36; L65; L44; L45; L29; L35; |
| | | L41; L45; L62 | | L32; L40; L41; L8; L42; L33 |
| 285 | SFKELLVY | L72; L71; L43; L26; L12; | IATNGPLKV | L73; L70; L69; L47; L58; L64; L24; |
| | | L14; L59; L66 | | L50; L63; L49; L20; L67; L61 |
| 286 | SFKWDLTAF | L43; L72; L71; L9; L8; | IIFLEGETL | L60; L28; L26; L68; L16; L67; L34; |
| | | L26; L10; L58 | | L61; L31; L53; L59; L36; L54 |
| 287 | SGKPVPYCY | L14; L66; L65; L43; L26; | ISNSWLMWL | L73; L61; L55; L60; L53; L59; L58; |
| | | L12; L69; L13 | | L74; L69; L70; L57; L4; L64 |
| 288 | SLDTYPSL | L63; L68; L5; L62; L74; | KENDSKEGF | L33; L41; L36; L16; L40; L37; L29; |
| | | L2; L28; L57 | | L26; L65; L27; L8; L38; L54 |
| 289 | SLPGVFCGV | L5; L2; L3; L57; L24; L4; | KIQEGVVDY | L14; L26; L16; L18; L65; L43; L13; |
| | | L20; L11 | | L6; L54; L12; L58; L27; L72 |
| 290 | SNYLKRRVV | L73; L25; L48; L64; L23; | KMVSLLSVL | L44; L69; L16; L56; L8; L73; L67; |
| | | L45; L46; L69 | | L3; L53; L36; L4; L27; L5 |
| 291 | SPYNSQNAV | L51; L47; L22; L50; L39; | KPVPEVKIL | L39; L32; L56; L69; L22; L31; L65; |
| | | L25; L28; L32 | | L64; L44; L66; L55; L49; L47 |
| 292 | SSDNIALL | L63; L62; L68; L74; L1; | KRVDWTIEY | L65; L64; L66; L30; L26; L43; L27; |
| | | L18; L60; L73 | | L16; L69; L14; L58; L12; L18 |
| 293 | SSVELKHF | L59; L52; L58; L54; L53; | KVDGVDVELF | L63; L54; L52; L18; L53; L5; L16; |
| | | L10; L43; L70 | | L68; L49; L62; L34; L1; L12 |
| 294 | STSAFVETV | L20; L70; L73; L3; L58; | KVVKVTIDY | L26; L14; L54; L18; L12; L16; L58; |
| | | L69; L4; L64 | | L13; L6; L52; L27; L11; L53 |
| 295 | SVCLGSLIY | L18; L12; L1; L14; L26; | LAWPLIVTA | L50; L69; L31; L47; L49; L51; L32; |
| | | L11; L7; L6 | | L52; L67; L70; L61; L5; L43 |
| 296 | SVSPKLFIR | L15; L7; L21; L19; L17; | LAWPLIVTAL | L67; L31; L61; L60; L68; L69; L74; |
| | | L6; L20; L11 | | L32; L47; L57; L56; L59; L50 |
| 297 | SVVLLSVL | L59; L25; L67; L74; L23; | LEAPFLYLY | L29; L40; L41; L12; L31; L1; L46; |
| | | L60; L56; L68 | | L18; L42; L38; L26; L52; L14 |
| 298 | SYEDQDALF | L62; L8; L9; L12; L72; | LLLTILTSL | L4; L5; L2; L3; L44; L23; L6; L56; |
| | | L34; L71; L66 | | L74; L59; L57; L8; L16 |
| 299 | SYFIASFRL | L9; L8; L72; L71; L66; | LPFNDGVYF | L49; L31; L32; L56; L39; L50; L22; |
| | | L65; L62; L64 | | L47; L52; L58; L9; L34; L12 |
| 300 | SYFIASFRLF | L9; L8; L12; L72; L71; | LQAENVTGL | L44; L3; L26; L28; L4; L25; L58; |
| | | L40; L41; L52 | | L35; L27; L56; L34; L64; L24 |
| 301 | TAPHGHVMV | L57; L69; L70; L63; L64; | MADLVYAL | L68; L67; L63; L62; L74; L60; L61; |
| | | L47; L32; L20 | | L59; L23; L47; L32; L70; L33 |
| 302 | TAYANSVF | L70; L58; L47; L59; L54; | NAAIVLQL | L74; L70; L67; L69; L47; L25; L61; |
| | | L69; L31; L68 | | L59; L60; L64; L23; L31; L35 |
| 303 | TEILPVSM | L29; L45; L37; L36; L33; | NLHPDSATL | L28; L3; L57; L5; L35; L2; L4; L26; |
| | | L38; L25; L46 | | L43; L34; L23; L16; L66 |
| 304 | TEISFMLWC | L46; L45; L37; L41; L38; | NTASWFTAL | L20; L60; L61; L25; L10; L35; L67; |
| | | L42; L40; L36 | | L11; L70; L73; L59; L28; L16 |
| 305 | TEVPANSTVL | L36; L37; L41; L45; L40; | NYSGVVTTV | L64; L20; L66; L35; L8; L72; L9; |
| | | L29; L28; L38 | | L24; L3; L71; L28; L70; L62 |
| 306 | TEVPVAIHA | L38; L46; L42; L37; L29; | QTFSVLACY | L11; L14; L12; L10; L6; L1; L18; |
| | | L41; L36; L40 | | L16; L52; L58; L69; L54; L70 |
| 307 | TGVEHVTF | L29; L23; L54; L26; L25; | QWLPTGTLL | L9; L8; L28; L34; L40; L36; L41; |
| | | L33; L49; L27 | | L74; L62; L61; L71; L22; L30 |
| 308 | TLMNVLTLV | L2; L4; L3; L5; L24; L20; | RIQPGQTF | L54; L26; L16; L55; L33; L39; L53; |
| | | L6; L64 | | L58; L43; L60; L59; L9; L72 |
| 309 | TLVTMPLGY | L12; L6; L26; L18; L16; | RTFKVSIWNL | L53; L44; L65; L20; L16; L13; L21; |
| | | L10; L14; L1 | | L54; L55; L56; L73; L6; L7 |
| 310 | TSDLATNNL | L68; L63; L1; L62; L60; | RTIKGTHHW | L54; L53; L16; L52; L10; L55; L49; |
| | | L74; L67; L34 | | L14; L11; L18; L21; L8; L73 |
| 311 | TTDPSFLGR | L1; L7; L19; L17; L15; | RTILGSALL | L53; L73; L55; L54; L13; L16; L60; |
| | | L18; L21; L6 | | L20; L14; L18; L36; L52; L44 |
| 312 | TVEELKKLL | L32; L39; L63; L23; L20; | SAPHGVVF | L59; L58; L63; L57; L66; L49; L70; |
| | | L1; L5; L68 | | L43; L72; L56; L69; L62; L71 |
| 313 | TVKNGSIHL | L13; L20; L10; L58; L56; | SAVVLLIL | L59; L74; L61; L60; L69; L67; L70; |
| | | L39; L74; L26 | | L47; L73; L25; L23; L31; L68 |
| 314 | TVKNGSIHLY | L14; L11; L10; L26; L12; | SEYDYVIF | L29; L33; L37; L27; L41; L45; L40; |
| | | L16; L52; L18 | | L43; L36; L25; L38; L66; L65 |
| 315 | TYLEGSVRV | L8; L2; L9; L71; L4; L72; | SSDNIALLV | L73; L18; L1; L63; L68; L24; L58; |
| | | L45; L62 | | L5; L70; L64; L62; L20; L69 |
| 316 | VAAGLEAPFL | L74; L67; L58; L60; L61; | TIVEVQPQL | L4; L5; L20; L60; L67; L28; L2; |
| | | L70; L69; L31 | | L61; L32; L74; L39; L8; L56 |
| 317 | VAGGIVAIV | L70; L69; L58; L64; L67; | TQAPTHLSV | L24; L35; L28; L48; L3; L25; L69; |
| | | L20; L47; L73 | | L34; L73; L58; L64; L26; L4 |
| 318 | VAVKMFDAY | L59; L43; L31; L67; L58; | VAIVVTCL | L74; L61; L60; L59; L70; L67; L69; |
| | | L12; L14; L49 | | L47; L68; L63; L72; L23; L62 |
| 319 | VAYRKVLLR | L19; L69; L17; L15; L47; | VASQSIIAY | L58; L70; L69; L31; L59; L49; L43; |
| | | L7; L6; L21 | | L26; L18; L12; L27; L14; L52 |
| 320 | VEEVTTTL | L37; L45; L25; L33; L36; | VLMDGSIIQF | L26; L16; L9; L8; L2; L11; L43; L4; |
| | | L48; L29; L62 | | L10; L27; L6; L12; L5 |
| 321 | VEIIKSQDL | L37; L36; L45; L40; L25; | VPMEKLKTL | L39; L23; L22; L56; L32; L47; L25; |
| | | L33; L41; L29 | | L31; L49; L8; L4; L3; L51 |
| 322 | VEKGVLPQL | L37; L36; L39; L45; L13; | VSTSGRWVL | L55; L70; L60; L61; L53; L69; L73; |
| | | L56; L29; L41 | | L65; L44; L16; L67; L58; L54 |
| 323 | VENPHLMGW | L41; L40; L33; L29; L45; | WTFGAGAAL | L61; L60; L20; L10; L59; L58; L16; |
| | | L52; L54; L38 | | L28; L70; L67; L53; L11; L57 |
| 324 | VLLAPLLSA | L2; L51; L6; L4; L3; L50; | YASAVVLLIL | L67; L69; L31; L60; L61; L59; L74; |
| | | L5; L46 | | L32; L70; L73; L58; L20; L47 |
| 325 | VMPLSAPTL | L57; L5; L9; L72; L66; | YIGDPAQL | L74; L60; L61; L67; L59; L66; L3; |
| | | L62; L71; L47 | | L73; L58; L4; L63; L62; L28 |
| 326 | VPFVVSTGY | L31; L49; L26; L43; L12; | YLQPRTFLL | L4; L74; L23; L2; L5; L64; L9; L1; |
| | | L65; L27; L56 | | L3; L61; L8; L66; L69 |
| 327 | VPLNIIPL | L47; L56; L50; L22; L23; | YVDNSSLTI | L68; L5; L34; L49; L1; L63; L24; |
| | | L39; L25; L31 | | L28; L67; L62; L32; L45; L61 |
| 328 | VQAGNVQL | L44; L35; L26; L28; L56; | YVLPNDDTL | L67; L60; L32; L61; L4; L74; L5; |
| | | L25; L74; L58 | | L2; L44; L34; L31; L53; L59 |
| 329 | VRDVLVRGF | L30; L62; L34; L49; L64; | AAIVFITL | L67; L59; L61; L74; L60; L70; L23; |
| | | L66; L65; L52 | | L69; L73; L68; L56; L58; L47; |
| | | | | L43 |
| 330 | VRIKIVQML | L64; L66; L35; L65; L25; | AELAKNVSL | L37; L33; L36; L44; L40; L42; L45; |
| | | L34; L30; L8 | | L41; L46; L23; L25; L38; L56; |
| | | | | L35 |
| 331 | VSDADSTLI | L63; L1; L68; L18; L53; | AHAEETRKL | L28; L34; L35; L64; L44; L10; L41; |
| | | L55; L62; L54 | | L27; L37; L40; L3; L55; L24; L4 |
| 332 | VSLVKPSFY | L43; L1; L52; L14; L12; | ALNTLVKQL | L4; L3; L2; L64; L5; L44; L16; L56; |
| | | L18; L58; L54 | | L57; L39; L13; L26; L74; L69 |
| 333 | VSSPDAVTAY | L18; L43; L1; L52; L26; | ANKDGIIWV | L13; L3; L43; L51; L69; L24; L70; |
| | | L14; L54; L53 | | L73; L64; L4; L45; L2; L42; L58 |
| 334 | VTPSGTWLTY | L18; L1; L11; L43; L12; | ASAVVLLIL | L73; L69; L61; L74; L59; L67; L60; |
| | | L16; L10; L52 | | L70; L53; L55; L56; L44; L64; |
| | | | | L54 |
| 335 | VVDSYYSL | L63; L68; L67; L62; L74; | ASFSTFKCY | L14; L26; L27; L70; L69; L43; L58; |
| | | L60; L5; L1 | | L18; L13; L16; L10; L7; L52; L1 |
| 336 | VVFDEISMA | L51; L3; L11; L13; L2; | EEFEPSTQY | L29; L41; L11; L40; L27; L26; L45; |
| | | L69; L24; L20 | | L42; L46; L10; L65; L37; L31; |
| | | | | L36 |
| 337 | VVQEGVLTA | L51; L3; L2; L50; L13; | FAFPFTIYS | L58; L70; L69; L59; L74; L64; L60; |
| | | L42; L7; L38 | | L61; L31; L67; L47; L50; L43; |
| | | | | L20 |
| 338 | VYADSFVIR | L17; L15; L9; L19; L21; | FAMQMAYRF | L58; L59; L49; L69; L53; L32; L74; |
| | | L8; L66; L7 | | L31; L12; L60; L70; L61; L63; |
| | | | | L54 |
| 339 | VYANGGKGF | L9; L8; L72; L71; L43; | FLFVAAIFY | L12; L26; L59; L69; L1; L31; L6; |
| | | L26; L12; L66 | | L43; L72; L18; L71; L14; L2; L64 |
| 340 | VYDDGARRV | L62; L64; L63; L45; L5; | FPFTIYSLL | L31; L32; L56; L47; L49; L22; L50; |
| | | L72; L71; L8 | | L64; L69; L44; L58; L39; L20; |
| | | | | L61 |
| 341 | VYNPFMIDV | L64; L72; L62; L71; L8; | FSNSGSDVL | L61; L60; L67; L58; L73; L70; L55; |
| | | L66; L73; L9 | | L59; L68; L74; L57; L53; L32; |
| | | | | L69 |
| 342 | VYRGTTTY | L72; L71; L66; L26; L43; | HFYWFFSNY | L12; L13; L14; L72; L71; L27; L49; |
| | | L27; L65; L14 | | L26; L43; L59; L65; L29; L11; |
| | | | | L10 |
| 343 | VYSTGSNVF | L9; L8; L72; L71; L66; | HSLSHFVNL | L73; L53; L61; L60; L67; L74; L44; |
| | | L62; L65; L53 | | L13; L55; L65; L54; L20; L59; |
| | | | | L69 |
| 344 | VYSVIYLY | L66; L12; L14; L72; L9; | IASEFSSL | L61; L67; L60; L59; L70; L57; L74; |
| | | L62; L71; L18 | | L68; L47; L63; L69; L58; L66; |
| | | | | L31 |
| 345 | VYSVIYLYL | L9; L66; L8; L64; L72; | ISTSHKLVL | L73; L61; L60; L69; L67; L53; L70; |
| | | L62; L71; L73 | | L59; L74; L65; L55; L54; L68; |
| | | | | L58 |
| 346 | WLDMVDTSL | L5; L68; L63; L74; L2; | IVNNWLKQL | L4; L64; L39; L73; L60; L58; L3; |
| | | L32; L62; L1 | | L16; L5; L56; L10; L26; L59; L69 |
| 347 | WNLDYIINL | L4; L65; L73; L74; L44; | IVSTIQRKY | L14; L12; L52; L58; L26; L55; L18; |
| | | L2; L13; L8 | | L64; L49; L54; L11; L6; L16; L13 |
| 348 | WPLIVTAL | L23; L47; L25; L22; L56; | KFKEGVEF | L62; L72; L71; L9; L43; L8; L26; |
| | | L31; L50; L39 | | L55; L27; L39; L56; L53; L23; L66 |
| 349 | YEDLLIRKS | L45; L46; L29; L41; L37; | KIADYNYKL | L16; L4; L2; L44; L73; L54; L24; |
| | | L38; L40; L42 | | L58; L8; L5; L20; L53; L3; L66 |
| 350 | YFTSDYYQL | L8; L9; L32; L72; L71; | KNFKSVLYY | L65; L12; L27; L69; L16; L6; L18; |
| | | L62; L67; L44 | | L14; L73; L26; L58; L52; L54; L13 |
| 351 | YGIATVREV | L64; L48; L69; L3; L73; | KPNELSRVL | L39; L56; L55; L32; L22; L65; L51; |
| | | L47; L46; L70 | | L44; L64; L31; L73; L66; L49; |
| | | | | L16 |
| 352 | YHDVRVVL | L35; L28; L62; L68; L34; | KQFDTYNLW | L16; L54; L44; L52; L24; L27; L26; |
| | | L63; L60; L25 | | L49; L8; L21; L14; L9; L13; L15 |
| 353 | YIIKLIFLWL | L67; L60; L74; L73; L61; | KSDGTGTIY | L18; L54; L1; L14; L63; L53; L27; |
| | | L10; L5; L11 | | L58; L55; L65; L68; L26; L52; L70 |
| 354 | YIINLIIKN | L69; L24; L11; L12; L4; | KVAGFAKFL | L73; L64; L55; L58; L69; L20; L13; |
| | | L58; L10; L19 | | L16; L74; L60; L39; L53; L56; |
| | | | | L44 |
| 355 | YIINLIIKNL | L10; L60; L3; L69; L4; | LQLPQGTTL | L44; L27; L61; L25; L26; L8; L28; |
| | | L20; L11; L5 | | L35; L60; L34; L4; L72; L71; L9 |
| 356 | YKVYYGNAL | L44; L67; L66; L61; L27; | LTNIFGTVY | L1; L26; L59; L18; L52; L58; L31; |
| | | L60; L71; L72 | | L12; L14; L53; L43; L65; L55; L6 |
| 357 | YLDGADVTKI | L5; L63; L2; L68; L62; L3; | NSFTRGVYY | L70; L69; L58; L1; L18; L14; L49; |
| | | L1; L24 | | L27; L52; L11; L13; L12; L54; L29 |
| 358 | YLNTLTLAV | L3; L4; L2; L5; L73; L24; | PANSTVLSF | L58; L49; L59; L43; L70; L32; L31; |
| | | L57; L64 | | L54; L69; L60; L9; L52; L65; L74 |
| 359 | YLQPRTFL | L74; L23; L62; L59; L2; | RQLLFVVEV | L48; L24; L73; L44; L4; L27; L13; |
| | | L72; L4; L61 | | L33; L30; L26; L2; L64; L69; L37 |
| 360 | YLYLTFYL | L74; L23; L59; L67; L68; | RSKNPLLY | L18; L55; L1; L14; L52; L13; L54; |
| | | L62; L2; L4 | | L70; L58; L26; L43; L53; L73; L69 |
| 361 | YPNASFDNF | L49; L31; L32; L22; L39; | SAYENFNQH | L59; L69; L49; L67; L58; L60; L7; |
| | | L47; L56; L9 | | L70; L43; L11; L61; L10; L6; L19 |
| 362 | YPSLETIQI | L49; L47; L32; L31; L22; | SCNNYMLTY | L43; L12; L26; L27; L16; L58; L18; |
| | | L50; L39; L34 | | L59; L14; L72; L49; L71; L29; |
| | | | | L10 |
| 363 | YRYNLPTM | L66; L59; L25; L28; L48; | SGVPVVDSY | L26; L14; L65; L31; L29; L66; L27; |
| | | L47; L35; L64 | | L12; L54; L69; L43; L52; L49; |
| | | | | L11 |
| 364 | YSGVVTTVM | L59; L70; L61; L43; L58; | SSAKSASVY | L58; L26; L18; L14; L1; L52; L54; |
| | | L73; L60; L69 | | L27; L43; L70; L53; L69; L10; L11 |
| 365 | YSSANNCTF | L58; L54; L53; L70; L34; | SSLPSYAAF | L43; L52; L59; L53; L27; L54; L61; |
| | | L60; L55; L52 | | L60; L10; L26; L16; L9; L58; L14 |
| 366 | YTVEEAKTV | L70; L69; L20; L58; L64; | SSVELKHFF | L53; L54; L58; L52; L49; L69; L60; |
| | | L73; L10; L11 | | L73; L40; L14; L55; L11; L70; L9 |
| 367 | YVYIGDPAQL | L3; L60; L67; L74; L20; | TANPKTPKY | L58; L70; L49; L59; L31; L43; L69; |
| | | L61; L28; L59 | | L14; L12; L54; L52; L11; L32; |
| | | | | L18 |
| 368 | YYHTTDPSF | L71; L66; L9; L72; L8; | TIKPVTYKL | L39; L58; L23; L8; L3; L4; L5; L20; |
| | | L62; L12; L32 | | L66; L74; L26; L10; L13; L16 |
| 369 | YYPSARIVY | L66; L71; L72; L12; L9; | TILTSLLVL | L4; L73; L8; L61; L23; L60; L6; |
| | | L64; L27; L8 | | L59; L67; L25; L35; L16; L74; L10 |
| 370 | YYSLLMPIL | L66; L9; L64; L62; L67; | TLQPVSEL | L23; L4; L3; L74; L57; L72; L28; |
| | | L72; L28; L71 | | L62; L71; L26; L2; L68; L66; L59 |
| 371 | AAFHQECSL | L67; L69; L32; L70; L61; | TVYEKLKPV | L3; L20; L23; L2; L10; L11; L5; L4; |
| | | L44; L60; L74; L58 | | L51; L43; L64; L39; L48; L69 |
| 372 | AARVVRSIF | L43; L58; L59; L52; L70; | VAMPNLYKM | L58; L70; L69; L59; L32; L61; L52; |
| | | L69; L27; L26; L22 | | L31; L54; L53; L60; L63; L47; |
| | | | | L74 |
| 373 | AEIRASANL | L36; L37; L40; L45; L41; | VAPGTAVL | L57; L63; L59; L61; L67; L70; L68; |
| | | L42; L38; L33; L46 | | L74; L66; L60; L47; L62; L58; |
| | | | | L56 |
| 374 | AEIVDTVSA | L38; L42; L46; L37; L36; | VATSRTLSY | L58; L59; L70; L43; L69; L49; L12; |
| | | L45; L40; L41; L43 | | L18; L31; L52; L54; L61; L27; |
| | | | | L14 |
| 375 | AEVQIDRLI | L40; L45; L41; L37; L36; | VAYRKVLL | L74; L23; L47; L67; L70; L63; L60; |
| | | L38; L42; L55; L46 | | L61; L69; L59; L68; L58; L56; |
| | | | | L53 |
| 376 | AGLEAPFLY | L12; L14; L7; L40; L16; | VLSDRELHL | L74; L2; L4; L5; L3; L57; L73; L64; |
| | | L6; L52; L27; L41 | | L9; L8; L16; L69; L24; L67 |
| 377 | ALDISASIV | L63; L18; L5; L68; L62; | VMYMGTLSY | L43; L26; L27; L59; L6; L16; L18; |
| | | L1; L2; L24; L28 | | L14; L71; L72; L12; L69; L1; L49 |
| 378 | ALVYFLQSI | L24; L3; L4; L5; L10; L2; | VSDIDITFL | L63; L68; L74; L53; L1; L62; L18; |
| | | L16; L45; L64 | | L60; L73; L67; L34; L61; L5; L58 |
| 379 | ASFDNFKF | L54; L55; L53; L52; L70; | VSIINNTVY | L26; L43; L52; L54; L18; L27; L1; |
| | | L58; L69; L16; L26 | | L53; L58; L70; L14; L69; L55; L59 |
| 380 | ASLNGVTLI | L24; L53; L55; L73; L52; | VTDVTQLYL | L68; L1; L63; L62; L18; L60; L67; |
| | | L48; L7; L16; L34 | | L73; L53; L61; L74; L5; L58; L70 |
| 381 | AVLDMCASL | L4; L16; L67; L10; L61; | VVFLHVTYV | L3; L64; L69; L47; L20; L73; L58; |
| | | L5; L44; L72; L2 | | L5; L4; L13; L2; L33; L48; L67 |
| 382 | AVMYMGTLSY | L26; L18; L6; L12; L16; | VYIGDPAQL | L9; L8; L72; L71; L74; L62; L66; |
| | | L1; L14; L10; L7 | | L67; L3; L28; L64; L60; L34; L13 |
| 383 | CPAEIVDTV | L47; L20; L50; L39; L32; | YADVFHLY | L1; L18; L59; L58; L49; L68; L63; |
| | | L51; L49; L31; L3 | | L70; L31; L62; L12; L60; L47; L54 |
| 384 | CSFGGVSVI | L70; L69; L48; L55; L65; | YIKWDLLKY | L58; L43; L12; L26; L11; L10; L59; |
| | | L16; L10; L53; L64 | | L14; L1; L13; L18; L69; L6; L16 |
| 385 | DLDDFSKQL | L5; L2; L63; L68; L10; | YPIIGDEL | L23; L56; L47; L22; L32; L67; L31; |
| | | L62; L11; L1; L35 | | L50; L61; L71; L59; L39; L25; |
| | | | | L72 |
| 386 | DLFMRIFTI | L23; L47; L25; L24; L20; | AAISDYDYY | L58; L14; L43; L12; L52; L69; L18; |
| | | L3; L10; L4; L16 | | L49; L11; L67; L54; L70; L10; |
| | | | | L31; L27 |
| 387 | DLTKGPHEF | L11; L10; L49; L16; L9; | ADAGFIKQY | L26; L69; L27; L14; L40; L70; L10; |
| | | L23; L74; L26; L29 | | L29; L43; L41; L11; L65; L58; |
| | | | | L16; L64 |
| 388 | DTVIEVQGY | L11; L10; L52; L29; L49; | ASRELKVTF | L53; L52; L43; L26; L54; L58; L27; |
| | | L26; L19; L65; L1 | | L65; L16; L55; L13; L69; L56; |
| | | | | L44; L70 |
| 389 | DVLLPLTQY | L11; L10; L17; L29; L6; | CQYLNTLTL | L48; L25; L44; L27; L26; L24; L35; |
| | | L31; L26; L14; L12 | | L28; L61; L34; L16; L73; L69; |
| | | | | L67; L59 |
| 390 | EAARVVRSI | L20; L10; L47; L49; L32; | DAALALLLL | L47; L74; L31; L67; L60; L73; L61; |
| | | L19; L69; L70; L64 | | L59; L69; L20; L64; L70; L58; |
| | | | | L23; L25 |
| 391 | EAFEKMVSLL | L20; L10; L11; L69; L70; | DAVRDPQTL | L32; L74; L49; L67; L60; L25; L53; |
| | | L31; L23; L74; L32 | | L31; L10; L47; L20; L70; L61; |
| | | | | L69; L58 |
| 392 | EAFLIGCNY | L31; L11; L43; L49; L19; | DVTDVTQLY | L11; L10; L12; L14; L49; L19; L20; |
| | | L58; L17; L12; L70 | | L31; L26; L1; L18; L58; L52; L29; |
| | | | | L16 |
| 393 | EIAIILASF | L11; L10; L20; L26; L40; | FAPSASAF | L57; L59; L58; L72; L43; L63; L61; |
| | | L19; L41; L43; L23 | | L71; L60; L70; L66; L74; L47; |
| | | | | L68; L62 |
| 394 | ELDSFKEEL | L5; L68; L23; L63; L1; L2; | FVDGVPFVV | L5; L63; L68; L2; L67; L20; L58; |
| | | L34; L20; L62 | | L62; L69; L32; L73; L50; L1; L64; |
| | | | | L61 |
| 395 | ERSEKSYEL | L35; L66; L65; L25; L34; | FVSNGTHWF | L58; L49; L59; L60; L16; L10; L11; |
| | | L23; L64; L28; L20 | | L26; L32; L69; L74; L34; L12; |
| | | | | L61; L53 |
| 396 | ESLIDLQEL | L53; L23; L55; L25; L10; | GTFTCASEY | L14; L18; L26; L58; L12; L10; L27; |
| | | L67; L59; L20; L73 | | L16; L52; L11; L70; L54; L13; L1; |
| | | | | L7 |
| 397 | ETISLAGSY | L11; L10; L14; L1; L12; | GTITSGWTF | L53; L52; L54; L10; L55; L11; L26; |
| | | L20; L19; L40; L18 | | L27; L58; L16; L34; L9; L12; L60; |
| | | | | L8 |
| 398 | FADDLNQLT | L68; L67; L63; L1; L49; | HAASGNLL | L74; L61; L60; L70; L58; L55; L63; |
| | | L31; L32; L58; L70 | | L56; L67; L44; L54; L59; L49; |
| | | | | L23; L32 |
| 399 | FLAFLLFLV | L2; L4; L3; L20; L64; L5; | HCANFNVLF | L34; L53; L31; L44; L12; L52; L8; |
| | | L24; L12; L69 | | L9; L58; L40; L35; L49; L54; L28; |
| | | | | L26 |
| 400 | FLLPSLATV | L3; L2; L4; L5; L59; L50; | IAFGGCVFSY | L31; L70; L69; L58; L49; L43; L12; |
| | | L47; L23; L24 | | L16; L27; L26; L52; L14; L54; |
| | | | | L59; L18 |
| 401 | FLLVTLAIL | L2; L4; L74; L59; L5; L61; | IAIPTNFTI | L70; L47; L54; L67; L8; L69; L61; |
| | | L62; L23; L3 | | L49; L32; L52; L53; L34; L68; L55 |
| | | | | L9 |
| 402 | FLTENLLL | L74; L2; L61; L68; L62; | KADETQAL | L68; L63; L67; L61; L60; L62; L54; |
| | | L4; L23; L5; L59 | | L70; L59; L57; L55; L74; L43; L5; |
| | | | | L56 |
| 403 | FLYIIKLIF | L59; L12; L69; L43; L49; | KALNLGETF | L53; L52; L54; L16; L8; L55; L58; |
| | | L26; L74; L23; L16 | | L9; L27; L31; L26; L60; L49; L12; |
| | | | | L70 |
| 404 | FLYIIKLIFL | L2; L74; L4; L5; L3; L69; | KAPKEIIFL | L74; L57; L53; L69; L64; L63; L58; |
| | | L20; L64; L6 | | L44; L56; L5; L73; L70; L66; L61; |
| | | | | L68 |
| 405 | FQVTIAEIL | L44; L69; L67; L58; L28; | KHWPQIAQF | L16; L27; L53; L28; L34; L54; L52; |
| | | L61; L34; L35; L60 | | L8; L35; L26; L9; L65; L49; L44; |
| | | | | L33 |
| 406 | FSAVGNICY | L43; L58; L70; L1; L18; | KPVETSNSF | L54; L49; L39; L31; L56; L32; L27; |
| | | L69; L59; L52; L26 | | L22; L16; L8; L51; L53; L9; L26; |
| | | | | L65 |
| 407 | FTEERLKL | L63; L1; L60; L68; L62; | KSHKPPISF | L43; L53; L16; L54; L52; L55; L26; |
| | | L61; L73; L70; L59 | | L58; L70; L73; L27; L63; L60; |
| | | | | L69; L56 |
| 408 | FTIKGSFL | L58; L60; L61; L59; L74; | KVNSTLEQY | L14; L16; L54; L26; L18; L58; L11; |
| | | L70; L63; L55; L73 | | L13; L6; L52; L12; L27; L53; L10; |
| | | | | L49 |
| 409 | FTIYSLLL | L61; L73; L60; L1; L59; | LATALLTL | L59; L61; L47; L60; L70; L67; L74; |
| | | L70; L67; L55; L58 | | L69; L56; L23; L73; L31; L55; |
| | | | | L68; L58 |
| 410 | FVDGVPFV | L63; L68; L62; L5; L2; | LLFLVLIML | L5; L74; L4; L2; L3; L6; L64; L59; |
| | | L58; L74; L1; L47 | | L69; L56; L44; L23; L26; L31; L62 |
| 411 | FVKHKHAFL | L23; L58; L74; L60; L63; | LTDEMIAQY | L18; L1; L14; L11; L12; L49; L62; |
| | | L20; L61; L59; L69 | | L58; L53; L52; L70; L63; L5; L10; |
| | | | | L68 |
| 412 | FVNEFYAY | L59; L12; L66; L43; L29; | LTNDNTSRY | L1; L58; L18; L14; L11; L26; L12; |
| | | L26; L14; L1; L58 | | L16; L52; L10; L43; L70; L6; L54; |
| | | | | L49 |
| 413 | FVTVYSHLL | L60; L61; L74; L58; L64; | NYMPYFFTL | L66; L8; L9; L72; L71; L65; L35; |
| | | L20; L68; L73; L32 | | L67; L34; L61; L25; L60; L62; L64; |
| | | | | L4 |
| 414 | FYGGWHNML | L9; L66; L71; L72; L74; | RTIKVFTTV | L73; L13; L20; L24; L53; L54; L58; |
| | | L64; L8; L62; L25 | | L3; L16; L15; L45; L37; L48; L11; |
| | | | | L8 |
| 415 | FYLCFLAFL | L72; L71; L62; L9; L8; | SIIQFPNTY | L26; L58; L12; L43; L11; L16; L69; |
| | | L64; L66; L4; L59 | | L10; L14; L59; L29; L6; L27; L7; |
| | | | | L3 |
| 416 | FYLITPVHV | L62; L72; L71; L50; L9; | SILSPLYAF | L16; L59; L12; L10; L43; L4; L6; |
| | | L2; L47; L64; L4 | | L8; L26; L11; L7; L60; L9; L58; L30 |
| 417 | FYWFFSNYL | L72; L62; L71; L64; L9; | STGYHFREL | L65; L73; L60; L13; L61; L59; L67; |
| | | L66; L68; L61; L8 | | L64; L70; L10; L55; L39; L69; |
| | | | | L58; L20 |
| 418 | GDYGDAVVY | L27; L26; L14; L65; L31; | STQDLFLPF | L7; L12; L10; L11; L16; L73; L59; |
| | | L29; L49; L43; L12 | | L53; L60; L52; L58; L72; L9; L6; |
| | | | | L13 |
| 419 | GETLPTEVL | L36; L37; L45; L41; L29; | TIAEILLII | L20; L3; L24; L69; L4; L73; L10; |
| | | L44; L40; L46; L28 | | L64; L34; L67; L47; L6; L11; L2; |
| | | | | L16 |
| 420 | GIVGVLTL | L74; L67; L60; L65; L61; | TSNQVAVLY | L55; L14; L54; L53; L1; L18; L65; |
| | | L24; L48; L28; L59 | | L52; L12; L26; L58; L31; L49; L19 |
| | | | | L69 |
| 421 | GQINDMILSL | L44; L27; L24; L34; L28; | TTTIKPVTY | L52; L26; L12; L69; L18; L54; L70; |
| | | L3; L26; L35; L4 | | L55; L53; L14; L16; L1; L58; L10; |
| | | | | L49 |
| 422 | GSIPCSVCL | L74; L44; L61; L53; L55; | VAFNTLLF | L58; L70; L59; L69; L74; L47; L54; |
| | | L60; L54; L73; L28 | | L52; L60; L49; L12; L31; L9; L43; |
| | | | | L53 |
| 423 | GTYEGNSPF | L11; L53; L10; L16; L27; | VASLINTL | L70; L59; L74; L67; L60; L68; L47; |
| | | L26; L43; L52; L54 | | L61; L69; L23; L57; L64; L55; |
| | | | | L63; L58 |
| 424 | HADQLTPTW | L49; L54; L52; L53; L63; | VTIAEILLI | L73; L24; L53; L55; L20; L7; L8; |
| | | L32; L68; L31; L67 | | L52; L69; L70; L47; L34; L12; L10; |
| | | | | L9 |
| 425 | HEETIYNLL | L36; L37; L41; L45; L40; | VTRELMREL | L13; L55; L64; L70; L73; L53; L60; |
| | | L29; L44; L34; L38 | | L58; L59; L10; L39; L52; L43; |
| | | | | L69; L61 |
| 426 | HEIAWYTER | L46; L37; L29; L17; L15; | YIIKLIFLW | L12; L54; L10; L49; L52; L16; L69; |
| | | L21; L38; L45; L36 | | L4; L58; L9; L8; L24; L11; L74; |
| | | | | L19 |
| 427 | HEVLLAPLL | L29; L37; L36; L41; L40; | YLYALVYFL | L2; L4; L74; L64; L3; L5; L69; L67; |
| | | L45; L33; L28; L44 | | L20; L44; L24; L73; L62; L61; |
| | | | | L66 |
| 428 | HHANEYRLY | L34; L28; L14; L11; L10; | YPDPSRIL | L32; L39; L22; L47; L62; L65; L68; |
| | | L27; L18; L12; L49 | | L63; L49; L31; L60; L61; L66; |
| | | | | L28; L23 |
| 429 | HSQLGGLHL | L60; L61; L73; L55; L53; | AALTNNVAF | L52; L59; L54; L53; L31; L58; L60; |
| | | L18; L68; L59; L63 | | L27; L61; L12; L71; L32; L43; |
| | | | | L72; L56; L40 |
| 430 | HTIDGSSGV | L11; L20; L10; L13; L58; | AANTVIWDY | L58; L14; L69; L43; L12; L65; L31; |
| | | L28; L24; L3; L73 | | L18; L26; L70; L49; L66; L59; |
| | | | | L52; L54; L27 |
| 431 | HVMSKHTDF | L11; L10; L49; L26; L23; | AVRDPQTL | L43; L55; L56; L3; L39; L58; L65; |
| | | L12; L58; L8; L53 | | L26; L67; L59; L13; L23; L66; L22 |
| | | | | L60; L63 |
| 432 | IAKNTVKSV | L70; L69; L58; L43; L47; | EYADVFHLY | L12; L9; L14; L8; L11; L66; L10; |
| | | L64; L39; L13; L3 | | L17; L20; L31; L40; L41; L65; L1; |
| | | | | L19; L72 |
| 433 | IAQYTSAL | L59; L61; L60; L67; L68; | FGDDTVIEV | L2; L5; L63; L62; L68; L67; L64; |
| | | L57; L71; L70; L72 | | L69; L35; L58; L70; L61; L32; L73; |
| | | | | L47; L49 |
| 434 | ICAPLTVF | L59; L26; L53; L9; L54; | FLAFVVFLL | L74; L4; L2; L20; L64; L3; L5; L69; |
| | | L58; L52; L27; L29 | | L68; L61; L44; L1; L9; L67; L66; |
| | | | | L34 |
| 435 | IIKLIFLWL | L13; L67; L60; L73; L39; | FLNRFTTTL | L74; L3; L5; L4; L2; L57; L59; L43; |
| | | L59; L58; L74; L23 | | L61; L32; L68; L26; L62; L44; |
| | | | | L64; L60 |
| 436 | ILDGISQY | L18; L1; L26; L63; L14; | FLYLYALVY | L12; L59; L69; L1; L43; L6; L26; |
| | | L27; L68; L43; L62 | | L27; L31; L18; L72; L14; L66; L71; |
| | | | | L16; L64 |
| 437 | ILLIIMRTF | L26; L55; L52; L16; L9; | FTISVTTEI | L61; L70; L20; L67; L60; L59; L73; |
| | | L8; L27; L12; L53 | | L10; L69; L53; L58; L3; L11; L49; |
| | | | | L55; L68 |
| 438 | ILMTARTVY | L26; L18; L16; L43; L12; | GEYSHVVAF | L37; L46; L33; L27; L29; L36; L41; |
| | | L14; L27; L72; L1 | | L38; L40; L45; L26; L42; L43; |
| | | | | L65; L49; L28 |
| 439 | INITRFQTL | L73; L23; L25; L8; L61; | HFIETISL | L72; L71; L23; L62; L35; L25; L48; |
| | | L60; L9; L32; L28 | | L44; L8; L28; L61; L37; L67; L29; |
| | | | | L59; L43 |
| 440 | ISMDNSPNL | L61; L60; L53; L73; L70; | IADKYVRNL | L63; L68; L74; L60; L32; L67; L61; |
| | | L54; L63; L58; L68 | | L58; L70; L49; L64; L62; L5; L47; |
| | | | | L65; L69 |
| 441 | ISNEKQEIL | L73; L54; L53; L60; L67; | IAQVDVVNF | L53; L49; L31; L52; L43; L58; L32; |
| | | L61; L55; L63; L70 | | L54; L26; L63; L70; L59; L74; L9; |
| | | | | L60; L69 |
| 442 | ISRQRLTKY | L43; L58; L26; L18; L52; | ITVEELKKL | L53; L10; L60; L73; L11; L20; L69; |
| | | L14; L53; L1; L27 | | L70; L64; L54; L55; L52; L8; L58; |
| | | | | L4; L5 |
| 443 | ITDAVDCAL | L68; L67; L63; L62; L60; | KFADDLNQL | L8; L9; L53; L4; L44; L72; L16; |
| | | L32; L1; L5; L61 | | L71; L64; L3; L56; L36; L34; L62; |
| | | | | L13; L2 |
| 444 | ITGGVVQL | L70; L73; L67; L53; L65; | KQGDDYVYL | L44; L65; L13; L73; L34; L26; L24; |
| | | L64; L54; L63; L74 | | L58; L27; L67; L69; L2; L66; L64; |
| | | | | L16; L4 |
| 445 | IVDEPEEHV | L5; L63; L68; L62; L2; | KSVNITFEL | L73; L53; L55; L54; L69; L70; L60; |
| | | L67; L20; L18; L28 | | L67; L16; L61; L44; L20; L58; L8; |
| | | | | L52; L56 |
| 446 | IVNSVLLF | L74; L54; L58; L73; L52; | KTLQPVSEL | L53; L16; L73; L65; L55; L60; L61; |
| | | L49; L12; L62; L26 | | L13; L54; L6; L67; L52; L4; L70; |
| | | | | L63; L8 |
| 447 | IWFLLLSV | L48; L47; L50; L25; L59; | KTYERHSL | L55; L16; L53; L70; L73; L54; L60; |
| | | L45; L33; L46; L62 | | L25; L65; L61; L67; L69; L39; |
| | | | | L59; L63; L71 |
| 448 | KHITSKETL | L34; L28; L35; L44; L36; | LAFVVFLLV | L47; L73; L69; L50; L59; L70; L20; |
| | | L8; L27; L16; L53 | | L61; L31; L64; L58; L49; L24; L5; |
| | | | | L32; L67 |
| 449 | KIFVDGVPF | L16; L53; L43; L54; L26; | LMDGSIIQF | L5; L62; L49; L34; L68; L63; L1; |
| | | L6; L52; L58; L56 | | L31; L74; L27; L26; L18; L58; L32; |
| | | | | L60; L9 |
| 450 | KLAKKFDTF | L16; L26; L53; L9; L8; | LSDDAVVCF | L63; L68; L62; L53; L1; L58; L54; |
| | | L54; L27; L10; L12 | | L60; L49; L52; L70; L74; L5; L18; |
| | | | | L69; L34 |
| 451 | KLLEQWNLV | L2; L4; L5; L3; L16; L73; | LSDRVVFVL | L68; L63; L65; L53; L1; L60; L67; |
| | | L24; L6; L13 | | L32; L74; L61; L55; L70; L54; L73 |
| | | | | L5; L62 |
| 452 | KLWAQCVQL | L2; L5; L4; L16; L44; L3; | LTIKKPNEL | L61; L60; L73; L58; L70; L53; L67; |
| | | L56; L6; L27 | | L20; L55; L23; L59; L69; L74; L8; |
| | | | | L16; L64 |
| 453 | KQLIKVTL | L44; L23; L33; L25; L48; | LTYTGAIKL | L55; L69; L53; L70; L60; L73; L64; |
| | | L27; L24; L65; L26 | | L58; L61; L74; L20; L13; L63; |
| | | | | L68; L47; L52 |
| 454 | KSHNIALIW | L52; L54; L53; L16; L55; | LVKPSFYVY | L26; L59; L14; L58; L12; L52; L13; |
| | | L73; L69; L18; L70 | | L18; L1; L43; L16; L31; L11; L66; |
| | | | | L10; L69 |
| 455 | KTIQPRVEK | L13; L7; L6; L16; L21; | MEIDFLEL | L29; L25; L59; L37; L45; L61; L67; |
| | | L15; L53; L19; L54 | | L73; L36; L41; L60; L40; L35; |
| | | | | L33; L48; L46 |
| 456 | KVSIWNLDY | L18; L16; L14; L26; L12; | MYASAVVLL | L9; L64; L62; L66; L8; L74; L72; |
| | | L6; L54; L1; L52 | | L71; L28; L20; L69; L31; L34; L56; |
| | | | | L35; L44 |
| 457 | LADNKFAL | L68; L63; L60; L67; L61; | REVRTIKVF | L37; L40; L29; L41; L36; L27; L33; |
| | | L23; L59; L32; L62 | | L45; L38; L46; L52; L26; L65; L8; |
| | | | | L53; L44 |
| 458 | LAIDAYPL | L61; L67; L59; L60; L74; | RVDGQVDLF | L54; L63; L18; L53; L62; L52; L74; |
| | | L70; L55; L47; L32 | | L5; L14; L16; L1; L68; L58; L12; |
| | | | | L49; L34 |
| 459 | LANTCTERL | L32; L49; L58; L74; L60; | SEFSSLPSY | L29; L41; L40; L46; L27; L36; L45; |
| | | L61; L63; L31; L70 | | L38; L37; L14; L26; L12; L16; |
| | | | | L31; L6; L11 |
| 460 | LAYYFMRF | L59; L47; L60; L58; L49; | SLPINVIVF | L26; L9; L57; L5; L27; L72; L64; |
| | | L70; L74; L69; L43 | | L11; L62; L66; L16; L8; L12; L71; |
| | | | | L10; L69 |
| 461 | LEAPFLYL | L29; L45; L74; L61; L33; | SSPDDQIGY | L43; L26; L11; L18; L12; L14; L58; |
| | | L59; L36; L37; L25 | | L27; L13; L1; L10; L7; L53; L66; |
| | | | | L52; L54 |
| 462 | LEIKDTEKY | L29; L41; L26; L49; L40; | TQYNRYLAL | L25; L23; L61; L35; L28; L24; L27; |
| | | L27; L36; L37; L45 | | L43; L44; L73; L67; L69; L10; L3; |
| | | | | L48; L71 |
| 463 | LFLAFVVFL | L72; L71; L62; L9; L8; L4; | TVYSHLLLV | L73; L3; L64; L47; L24; L20; L69; |
| | | L31; L64; L47 | | L6; L4; L58; L11; L5; L2; L48; L51; |
| | | | | L7 |
| 464 | LFLALITL | L71; L72; L47; L62; L56; | VAKSHNIAL | L43; L67; L60; L70; L58; L61; L59; |
| | | L23; L25; L8; L59 | | L68; L69; L23; L56; L39; L74; |
| | | | | L57; L63; L32 |
| 465 | LLFNKVTL | L23; L56; L4; L25; L2; | VENPDILRV | L45; L37; L73; L29; L38; L48; L24; |
| | | L44; L26; L74; L59 | | L42; L4; L36; L46; L40; L41; L33; |
| | | | | L3; L64 |
| 466 | LLLLFVTVY | L26; L12; L27; L6; L31; | VLLSVLQQL | L4; L2; L5; L3; L74; L8; L44; L16; |
| | | L59; L1; L16; L14 | | L56; L6; L9; L24; L53; L39; L55; |
| | | | | L59 |
| 467 | LMNVLTLVY | L18; L26; L31; L1; L12; | VVAFNTLLF | L12; L58; L9; L74; L52; L11; L18; |
| | | L43; L59; L16; L14 | | L6; L16; L26; L60; L1; L8; L49; |
| | | | | L31; L54 |
| 468 | LPINVIVF | L31; L47; L50; L56; L39; | VVFNGVSF | L26; L43; L58; L59; L54; L71; L52; |
| | | L32; L23; L49; L22 | | L72; L60; L53; L56; L16; L27; |
| | | | | L39; L55; L23 |
| 469 | LTKGPHEF | L58; L70; L55; L53; L52; | YAKPFLNKV | L69; L70; L58; L47; L3; L59; L64; |
| | | L59; L60; L43; L26 | | L73; L43; L50; L48; L20; L51; L67; |
| | | | | L5; L4 |
| 470 | LVIGAVIL | L74; L67; L60; L61; L59; | YASAVVLLI | L70; L47; L69; L64; L73; L74; L49; |
| | | L32; L55; L56; L69 | | L20; L67; L24; L31; L32; L61; |
| | | | | L34; L58; L55 |
| 471 | LVIGFLFL | L74; L60; L61; L73; L59; | YTVELGTEV | L20; L58; L61; L67; L70; L73; L69; |
| | | L67; L20; L58; L47 | | L11; L64; L5; L2; L10; L59; L50; |
| | | | | L60; L45 |
| 472 | LVNKFLAL | L59; L73; L60; L61; L23; | CVADYSVLY | L12; L11; L26; L58; L18; L6; L14; |
| | | L56; L67; L39; L74 | | L1; L7; L10; L16; L19; L31; L54; |
| | | | | L13; L69; L65 |
| 473 | LYLQYIRKL | L8; L64; L9; L71; L72; | DAFKLNIKL | L20; L74; L32; L47; L58; L69; L70; |
| | | L55; L65; L66; L4 | | L31; L25; L44; L49; L19; L53; |
| | | | | L64; L29; L56; L11 |
| 474 | LYSPIFLIV | L64; L73; L9; L47; L69; | ELFENKTTL | L23; L20; L2; L3; L10; L11; L4; |
| | | L8; L50; L20; L66 | | L44; L5; L25; L26; L16; L24; L34; |
| | | | | L35; L8; L56 |
| 475 | LYYPSARIV | L64; L47; L72; L69; L70; | FAMGIIAM | L59; L61; L67; L70; L58; L60; L69; |
| | | L71; L66; L62; L28 | | L74; L68; L31; L32; L50; L43; |
| | | | | L47; L63; L66; L72 |
| 476 | MEIDFLELA | L46; L42; L38; L41; L50; | FASEAARVV | L69; L70; L64; L61; L58; L59; L47; |
| | | L37; L40; L29; L45 | | L67; L32; L73; L50; L31; L57; |
| | | | | L49; L68; L60; L63 |
| 477 | MFDAYVNTF | L62; L34; L9; L72; L71; | FLTENLLLY | L12; L1; L6; L2; L18; L59; L3; L11; |
| | | L49; L32; L68; L8 | | L69; L26; L14; L4; L16; L64; L5; |
| | | | | L49; L10 |
| 478 | MIAQYTSAL | L61; L10; L60; L28; L59; | GWLIVGVAL | L44; L71; L56; L67; L72; L8; L36; |
| | | L3; L57; L67; L74 | | L35; L28; L61; L25; L37; L30; L34; |
| | | | | L60; L27; L23 |
| 479 | MMISAGFSL | L61; L60; L28; L35; L8; | HAFLCLFLL | L69; L32; L31; L47; L59; L64; L74; |
| | | L34; L32; L4; L72 | | L70; L20; L53; L50; L60; L44; |
| | | | | L67; L73; L58; L61 |
| 480 | MSFTVLCL | L65; L69; L70; L74; L73; | IAFGGCVF | L59; L58; L70; L60; L54; L69; L31; |
| | | L48; L61; L59; L60 | | L43; L47; L61; L52; L27; L26; |
| | | | | L53; L49; L74; L55 |
| 481 | MYTPHTVL | L66; L71; L72; L25; L9; | ICAPLTVFF | L9; L58; L53; L52; L69; L31; L34; |
| | | L62; L8; L74; L61 | | L12; L8; L74; L26; L54; L49; L64; |
| | | | | L27; L40; L62 |
| 482 | NASFDNFKF | L49; L31; L53; L12; L70; | ISDEVARDL | L55; L68; L60; L63; L62; L61; L53; |
| | | L32; L54; L58; L52 | | L1; L67; L64; L54; L73; L5; L74; |
| | | | | L65; L32; L70 |
| 483 | NESGLKTIL | L37; L36; L29; L41; L40; | KAYKIEELF | L54; L53; L58; L52; L49; L69; L55; |
| | | L45; L35; L42; L25 | | L74; L70; L27; L16; L26; L12; L9; |
| | | | | L31; L8; L63 |
| 484 | NLCPFGEVF | L43; L26; L16; L9; L8; | KEIDRLNEV | L45; L46; L37; L38; L3; L73; L36; |
| | | L27; L34; L11; L59 | | L33; L42; L24; L4; L25; L48; L2; |
| | | | | L64; L69; L29 |
| 485 | NLDYIINLI | L5; L24; L62; L68; L1; | KEIKESVQTF | L37; L36; L26; L40; L54; L8; L41; |
| | | L34; L2; L49; L63 | | L53; L27; L16; L33; L29; L52; L38; |
| | | | | L46; L45; L55 |
| 486 | NLNESLIDL | L4; L2; L5; L3; L57; L23; | KHIDAYKTF | L27; L34; L43; L16; L8; L53; L26; |
| | | L62; L74; L56 | | L54; L52; L28; L35; L9; L10; L55; |
| | | | | L58; L44; L41 |
| 487 | NLVIGFLFL | L74; L5; L61; L67; L2; | KLVNKFLAL | L73; L23; L16; L5; L4; L6; L3; L2; |
| | | L20; L73; L4; L8 | | L61; L44; L8; L67; L59; L10; L13; |
| | | | | L57; L56 |
| 488 | NSHEGKTF | L55; L43; L70; L26; L60; | LAILTALRL | L74; L60; L61; L67; L31; L69; L32; |
| | | L29; L54; L53; L59 | | L47; L59; L70; L73; L58; L49; |
| | | | | L68; L63; L64; L50 |
| 489 | NSIDAFKL | L55; L70; L60; L54; L73; | NIIIWFLLL | L73; L67; L60; L74; L61; L23; L8; |
| | | L67; L69; L35; L61 | | L34; L6; L20; L11; L69; L59; L5; |
| | | | | L28; L4; L25 |
| 490 | NSVLLFLAF | L60; L59; L67; L29; L52; | NTFTRLQSL | L25; L23; L10; L70; L20; L69; L64; |
| | | L40; L27; L73; L31 | | L60; L58; L16; L11; L3; L53; L73; |
| | | | | L61; L59; L57 |
| 491 | NTSNQVAVL | L20; L70; L35; L69; L28; | NVLEGSVAY | L12; L11; L26; L31; L10; L14; L27; |
| | | L67; L73; L53; L10 | | L16; L43; L6; L29; L71; L13; L7; |
| | | | | L72; L49; L54 |
| 492 | PYPDPSRIL | L66; L9; L8; L64; L72; | RIFTIGTVTL | L16; L44; L56; L26; L54; L13; L24; |
| | | 1; L65; L62; L57 | | L37; L21; L3; L39; L53; L4; L6; |
| | | | | L20; L5; L36 |
| 493 | QECVRGTTV | L45; L33; L38; L42; L48; | RMYIFFASF | L27; L16; L26; L43; L59; L53; L54; |
| | | L46; L25; L37; L29 | | L9; L65; L8; L12; L48; L72; L33; |
| | | | | L6; L14; L52 |
| 494 | QKFNGLTVL | L44; L27; L28; L64; L66; | RVVFNGVSF | L26; L54; L16; L53; L52; L43; L27; |
| | | L69; L25; L34; L35 | | L56; L44; L58; L60; L12; L11; |
| | | | | L39; L55; L8; L49 |
| 495 | QSAPHGVVFL | L73; L53; L69; L20; L74; | SAFYILPSI | L47; L69; L70; L20; L48; L50; L49; |
| | | L55; L64; L70; L61 | | L59; L32; L64; L4; L67; L31; L3; |
| | | | | L58; L24; L52 |
| 496 | REVRTIKV | L45; L33; L37; L48; L29; | SSSSDNIAL | L53; L56; L67; L57; L35; L44; L68; |
| | | L38; L46; L36; L24 | | L70; L20; L61; L32; L34; L60; |
| | | | | L73; L58; L74; L28 |
| 497 | RNAPRITF | L54; L65; L27; L33; L53; | TAFGLVAEW | L49; L69; L31; L54; L52; L10; L70; |
| | | L26; L55; L52; L16 | | L19; L67; L20; L58; L32; L64; |
| | | | | L53; L16; L47; L59 |
| 498 | RPDTRYVLM | L22; L39; L32; L18; L49; | TTIQTIVEV | L20; L73; L70; L64; L4; L69; L3; |
| | | L63; L56; L33; L62 | | L35; L58; L2; L24; L19; L11; L28; |
| | | | | L48; L10; L13 |
| 499 | RSDVLLPL | L68; L63; L73; L18; L1; | VARDLSLQF | L58; L59; L43; L49; L70; L53; L52; |
| | | L54; L60; L59; L62 | | L12; L26; L27; L60; L31; L69; L9; |
| | | | | L13; L11; L54 |
| 500 | RSLKVPATV | L73; L54; L53; L55; L24; | VPFWITIAY | L43; L31; L49; L50; L51; L26; L12; |
| | | L13; L48; L52; L16 | | L27; L56; L29; L32; L39; L69; |
| | | | | L72; L22; L47; L6 |
| 501 | RVDWTIEY | L18; L1; L65; L26; L54; | VVYRGTTTY | L26; L43; L11; L58; L27; L14; L10; |
| | | L68; L58; L63; L14 | | L6; L12; L49; L16; L59; L54; L18; |
| | | | | L52; L53; L69 |
| 502 | RVLGLKTL | L39; L55; L56; L16; L53; | VYDPLQPEL | L62; L9; L67; L66; L8; L68; L5; L6; |
| | | L54; L73; L23; L60 | | L63; L72; L28; L71; L60; L74; L64; |
| | | | | L61; L1; L34 |
| 503 | RVWTLMNVL | L16; L44; L53; L65; L13; | YCYDTNVL | L59; L25; L67; L61; L66; L71; L35; |
| | | L56; L73; L68; L39 | | L72; L60; L70; L28; L69; L47; |
| | | | | L64; L48; L68; L58 |
| 504 | SADAQSFL | L63; L68; L74; L62; L60; | YLFDESGEF | L26; L43; L10; L27; L11; L12; L59; |
| | | L58; L70; L67; L61 | | L9; L49; L2; L72; L3; L5; L16; |
| | | | | L66; L58; L8 |
| 505 | SEAARVVRS | L42; L46; L33; L38; L40; | YTPSKLIEY | L58; L11; L59; L12; L10; L1; L69; |
| | | L29; L41; L45; L37 | | L43; L64; L70; L66; L26; L72; L18; |
| | | | | L57; L71; L6 |
| 506 | SEAARVVRSI | L40; L45; L41; L42; L38; | AMDEFIERY | L18; L1; L27; L12; L5; L26; L43; |
| | | L37; L36; L33; L46 | | L14; L49; L16; L41; L40; L29; L34; |
| | | | | L2; L54; L62; L10 |
| 507 | SELVIGAVI | L45; L41; L36; L40; L37; | DSKEGFFTY | L10; L11; L43; L55; L26; L14; L29; |
| | | L42; L29; L38; L48 | | L13; L52; L53; L16; L58; L1; L70; |
| | | | | L18; L65; L17; L49 |
| 508 | SEVVLKKL | L29; L37; L45; L25; L33; | FADDLNQL | L67; L68; L63; L74; L59; L60; L61; |
| | | L41; L48; L36; L40 | | L58; L70; L62; L69; L5; L47; L32; |
| | | | | L64; L1; L31; L49 |
| 509 | SFLPGVYSV | L4; L72; L71; L2; L8; L48; | FAVDAAKAY | L59; L70; L58; L61; L43; L31; L69; |
| | | L35; L13; L9 | | L60; L67; L49; L12; L10; L71; |
| | | | | L66; L72; L32; L14; L64 |
| 510 | SFYYVWKSY | L13; L71; L43; L72; L27; | FTIGTVTL | L61; L67; L60; L59; L70; L58; L69; |
| | | L14; L12; L26; L69 | | L74; L20; L73; L68; L71; L63; |
| | | | | L53; L55; L62; L54; L72 |
| 511 | SHFAIGLALY | L12; L14; L41; L40; L34; | FTSDYYQLY | L58; L12; L1; L70; L64; L69; L18; |
| | | L27; L31; L18; L6 | | L14; L11; L49; L43; L10; L31; L59; |
| | | | | L52; L16; L66; L55 |
| 512 | SLSSTASAL | L57; L4; L28; L3; L44; L2; | HANEYRLYL | L73; L32; L61; L60; L67; L70; L69; |
| | | L74; L56; L72 | | L74; L58; L49; L59; L65; L57; |
| | | | | L64; L63; L31; L68; L43 |
| 513 | SPIFLIVAA | L51; L50; L22; L32; L42; | LAYILFTRF | L49; L59; L31; L47; L60; L52; L58; |
| | | L31; L56; L38; L39 | | L53; L69; L12; L74; L43; L70; |
| | | | | L26; L27; L50; L32; L61 |
| 514 | SSGDATTAY | L43; L26; L14; L58; L18; | LITGRLQSL | L23; L60; L59; L61; L58; L39; L3; |
| | | L1; L59; L10; L54 | | L4; L56; L25; L10; L74; L64; L57; |
| | | | | L5; L43; L32; L69 |
| 515 | SSGVTREL | L59; L65; L60; L61; L73; | RLNEVAKNL | L4; L2; L5; L13; L16; L44; L39; |
| | | L70; L43; L55; L48 | | L55; L57; L56; L3; L24; L26; L73; |
| | | | | L14; L21; L64; L6 |
| 516 | STDTGVEHV | L63; L18; L68; L1; L20; | SIIIGGAKL | L11; L28; L60; L10; L61; L58; L20; |
| | | L62; L5; L58; L2 | | L56; L63; L26; L24; L74; L34; L8; |
| | | | | L68; L4; L36; L37 |
| 517 | STFNVPMEK | L7; L13; L6; L19; L20; | TLNDLNETL | L4; L2; L5; L34; L16; L3; L8; L26; |
| | | L69; L17; L73; L21 | | L68; L57; L44; L35; L32; L9; L20; |
| | | | | L24; L62; L56 |
| 518 | TAAKLMVVI | L47; L70; L69; L49; L32; | TTADIVVF | L70; L65; L53; L69; L58; L52; L54; |
| | | L20; L67; L53; L64 | | L10; L20; L55; L26; L11; L59; |
| | | | | L35; L43; L29; L31; L66 |
| 519 | TCDGTTFTY | L18; L1; L65; L49; L14; | TTLPVNVAF | L53; L52; L54; L16; L31; L8; L12; |
| | | L34; L12; L27; L26 | | L9; L20; L59; L26; L35; L60; L55; |
| | | | | L6; L11; L40; L10 |
| 520 | TCFSTOFAF | L35; L16; L9; L34; L40; | VRFPNITNL | L66; L64; L65; L25; L30; L35; L34; |
| | | L27; L12; L8; L65 | | L74; L3; L28; L69; L4; L60; L44; |
| | | | | L8; L9; L61; L70 |
| 521 | TEVLTEEV | L33; L29; L45; L48; L37; | VRIQPGQTF | L34; L30; L43; L26; L8; L9; L35; |
| | | L38; L46; L25; L36 | | L66; L28; L65; L27; L53; L52; L49; |
| | | | | L58; L60; L12; L54 |
| 522 | TEVPANSTV | L45; L37; L38; L36; L46; | YAFEHIVYG | L70; L69; L58; L64; L61; L47; L59; |
| | | L29; L28; L41; L33 | | L49; L67; L74; L31; L60; L20; |
| | | | | L32; L50; L43; L73; L3 |
| 523 | TIDYTEISF | L68; L62; L63; L34; L32; | YLITPVHVM | L61; L32; L5; L69; L11; L70; L2; |
| | | L28; L1; L74; L5 | | L59; L67; L3; L28; L4; L26; L71; |
| | | | | L43; L72; L10; L27 |
| 524 | TLMNVLTL | L23; L4; L3; L2; L57; L72; | YVNTFSSTF | L58; L26; L10; L59; L43; L54; L11; |
| | | L74; L71; L68 | | L60; L49; L9; L12; L16; L32; L71; |
| | | | | L53; L27; L72; L52 |
| 525 | TLQPVSELL | L2; L4; L5; L28; L9; L8; | AIASEFSSL | L10; L3; L61; L60; L28; L16; L44; |
| | | L34; L74; L20 | | L58; L26; L4; L74; L56; L39; L22; |
| | | | | L6; L11; L67; L43; L57 |
| 526 | TMCDIRQLL | L64; L34; L16; L8; L5; | FAFPFTIY | L59; L70; L69; L58; L31; L47; L61; |
| | | L40; L20; L41; L4 | | L66; L74; L67; L60; L43; L27; |
| | | | | L12; L26; L29; L49; L64; L50 |
| 527 | TPHTVLQAV | L51; L50; L47; L39; L32; | FCYMHHMEL | L59; L61; L60; L67; L25; L69; L74; |
| | | L56; L23; L22; L24 | | L73; L58; L71; L70; L66; L43; |
| | | | | L28; L23; L64; L68; L72; L57 |
| 528 | TPINLVRDL | L32; L39; L64; L31; L56; | FVSEETGTL | L60; L61; L10; L32; L68; L20; L58; |
| | | L22; L49; L34; L47 | | L74; L67; L3; L5; L49; L11; L2; |
| | | | | L4; L44; L31; L57; L59 |
| 529 | TPVVQTIEV | L50; L47; L51; L32; L39; | FVVEVVDKY | L12; L31; L58; L11; L14; L69; L10; |
| | | L22; L49; L31; L56 | | L26; L49; L1; L20; L54; L59; L5; |
| | | | | L52; L29; L67; L66; L64 |
| 530 | TRPLLESEL | L64; L66; L57; L35; L65; | GADPIHSL | L68; L63; L67; L60; L74; L61; L70; |
| | | L34; L28; L62; L67 | | L23; L32; L55; L62; L59; L54; |
| | | | | L35; L58; L49; L69; L28; L66 |
| 531 | TTYPGQGL | L55; L25; L60; L61; L54; | KATEETFKL | L54; L53; L55; L69; L70; L60; L44; |
| | | L70; L28; L53; L20 | | L73; L63; L49; L24; L74; L32; |
| | | | | L58; L16; L8; L61; L68; L52 |
| 532 | TVIEVQGY | L11; L26; L10; L43; L14; | LALSKGVHF | L59; L58; L52; L60; L31; L12; L49; |
| | | L29; L54; L12; L55 | | L53; L61; L47; L74; L54; L32; L9; |
| | | | | L8; L43; L63; L70; L50 |
| 533 | TVVIGTSKF | L11; L26; L10; L49; L52; | LVAEWFLAY | L12; L59; L6; L10; L26; L1; L31; |
| | | L54; L16; L58; L53 | | L18; L11; L43; L16; L7; L58; L14; |
| | | | | L27; L49; L69; L52; L61 |
| 534 | TYFTQSRNL | L71; L72; L8; L64; L65; | LVSDIDITF | L49; L26; L58; L32; L52; L31; L5; |
| | | L9; L66; L13; L62 | | L12; L53; L16; L9; L20; L56; L54; |
| | | | | L69; L39; L60; L55; L34 |
| 535 | VADYSVLY | L1; L18; L63; L68; L62; | SEVGPEHSL | L37; L36; L29; L40; L41; L45; L28; |
| | | L58; L70; L54; L49 | | L44; L32; L42; L38; L67; L34; |
| | | | | L65; L33; L56; L35; L46; L61 |
| 536 | VAELEGIQY | L1; L31; L18; L27; L26; | SIIAYTMSL | L43; L60; L61; L4; L58; L10; L3; |
| | | L58; L49; L12; L70 | | L28; L73; L74; L59; L71; L16; L72; |
| | | | | L67; L5; L22; L57; L69 |
| 537 | VATAEAEL | L74; L59; L67; L61; L70; | SQLGGLHLL | L44; L4; L24; L34; L8; L30; L2; |
| | | L60; L63; L68; L56 | | L14; L27; L16; L35; L25; L58; L73; |
| | | | | L64; L26; L28; L56; L40 |
| 538 | VENPTIQKD | L37; L29; L45; L40; L41; | STNVTIATY | L14; L43; L11; L26; L10; L18; L16; |
| | | L36; L49; L38; L65 | | L58; L65; L54; L12; L52; L27; |
| | | | | L49; L1; L53; L7; L6; L70 |
| 539 | VIYLYLTF | L59; L43; L26; L60; L47; | SVVSKVVKV | L20; L73; L69; L3; L4; L58; L11; |
| | | L74; L48; L58; L23 | | L2; L64; L13; L5; L24; L10; L63; |
| | | | | L51; L49; L19; L7; L45 |
| 540 | VLIMLIIFW | L4; L69; L74; L2; L12; L5; | TTNGDFLHF | L53; L10; L73; L52; L11; L58; L16; |
| | | L3; L9; L6 | | L49; L8; L65; L9; L54; L12; L18; |
| | | | | L59; L26; L31; L1; L60 |
| 541 | VLKGVKLHY | L12; L26; L14; L43; L18; | AWPLIVTAL | L57; L62; L72; L71; L64; L67; L5; |
| | | L13; L16; L6; L1 | | L56; L9; L66; L33; L44; L40; L36; |
| | | | | L22; L68; L41; L39; L28; L8 |
| 542 | VLKKLKKSL | L23; L39; L43; L3; L56; | FVCNLLLLF | L12; L58; L69; L11; L74; L59; L31; |
| | | L26; L44; L13; L16 | | L52; L64; L5; L62; L34; L60; L73; |
| | | | | L49; L53; L10; L9; L1; L16 |
| 543 | VLTEEVVL | L74; L44; L62; L23; L67; | FVFLVLLPL | L59; L67; L61; L74; L60; L20; L69; |
| | | L2; L68; L63; L56 | | L64; L73; L31; L68; L6; L50; L5; |
| | | | | L4; L58; L3; L47; L2; L32 |
| 544 | VRSIFSRTL | L64; L65; L66; L25; L35; | HLVDFQVTI | L24; L20; L5; L3; L2; L49; L44; |
| | | L28; L34; L55; L30 | | L34; L4; L10; L16; L48; L8; L32; L69; |
| | | | | L64; L45; L53; L26; L35 |
| 545 | VSNGTHWF | L58; L54; L70; L55; L59; | HSIGFDYVY | L14; L54; L65; L26; L55; L18; L52; |
| | | L60; L43; L74; L26 | | L32; L11; L69; L53; L58; L70; |
| | | | | L43; L27; L29; L59; L10; L12; L49 |
| 546 | VSQPFLMDL | L73; L60; L59; L61; L69; | IVNGVRRSF | L16; L26; L52; L59; L60; L39; L43; |
| | | L70; L74; L53; L58 | | L58; L55; L10; L54; L53; L49; |
| | | | | L32; L12; L14; L11; L65; L27; L31 |
| 547 | VVADAVIKTL | L10; L3; L20; L39; L60; | KAIDGGVTR | L15; L19; L21; L17; L43; L54; L67; |
| | | L56; L16; L4; L11 | | L7; L61; L69; L53; L55; L13; L58; |
| | | | | L52; L70; L49; L60; L44; L27 |
| 548 | VVIGTSKFY | L14; L58; L26; L12; L11; | LLMPILTL | L4; L61; L74; L59; L3; L23; L2; |
| | | L13; L43; L27; L10 | | L55; L25; L47; L68; L73; L64; |
| | | | | L57; L43; L69; L26; L62; L67 |
| 549 | VVQQLPETY | L26; L14; L12; L16; L52; | LSFKELLVY | L69; L1; L58; L26; L52; L18; L59; |
| | | L18; L31; L29; L58 | | L53; L27; L54; L70; L31; L55; L65; |
| | | | | L16; L14; L43; L73; L12; L49 |
| 550 | VYFLQSINF | L9; L72; L71; L8; L62; | LWLLWPVTL | L62; L25; L9; L67; L60; L4; L8; |
| | | L12; L66; L26; L27 | | L31; L47; L61; L50; L71; L44; L55; |
| | | | | L56; L73; L23; L59; L74; L72 |
| 551 | WRNTNPIQL | L64; L66; L65; L35; L30; | NVIPTITQM | L11; L19; L74; L69; L10; L58; L28; |
| | | L25; L34; L28; L74 | | L5; L60; L32; L20; L31; L26; L29; |
| | | | | L17; L35; L49; L3; L34; L6 |
| 552 | WTLMNVLTL | L73; L16; L61; L60; L53; | QASLNGVTL | L32; L67; L61; L60; L56; L70; L68; |
| | | L55; L4; L69; L10 | | L44; L31; L55; L69; L58; L39; |
| | | | | L59; L22; L63; L57; L74; L28; L53 |
| 553 | YAADPAMHA | L61; L50; L69; L58; L60; | QSAPHGVVF | L53; L54; L52; L70; L69; L59; L43; |
| | | L59; L70; L43; L67 | | L55; L58; L65; L61; L26; L28; L9; |
| | | | | L60; L73; L27; L66; L11; L74 |
| 554 | YADSFVIRG | L74; L32; L63; L49; L68; | RTAPHGHVM | L53; L54; L70; L65; L73; L61; L55; |
| | | L69; L58; L70; L67 | | L52; L58; L16; L69; L18; L11; |
| | | | | L14; L60; L59; L21; L39; L15; L26 |
| 555 | YANGGKGF | L58; L59; L60; L43; L49; | SLIDFYLCF | L43; L26; L11; L12; L10; L8; L59; |
| | | L70; L61; L27; L26 | | L16; L5; L9; L27; L6; L69; L3; L49; |
| | | | | L4; L34; L2; L74; L72 |
| 556 | YCIDGALL | L61; L60; L59; L28; L71; | SSPDAVTAY | L43; L57; L72; L71; L26; L66; L10; |
| | | L74; L67; L72; L62 | | L58; L14; L11; L59; L1; L64; L69; |
| | | | | L12; L27; L7; L18; L65; L6 |
| 557 | YFKYWDQTY | L12; L43; L26; L72; L71; | SVGPKQASL | L20; L10; L39; L56; L43; L65; L28; |
| | | L58; L27; L14; L13 | | L58; L74; L67; L5; L44; L61; L16; |
| | | | | L4; L22; L3; L23; L60; L11 |
| 558 | YHPNCVNCL | L28; L64; L57; L35; L34; | TAQNSVRVL | L70; L32; L69; L61; L60; L64; L31; |
| | | L71; L72; L66; L62 | | L59; L39; L67; L28; L58; L55; |
| | | | | L68; L56; L57; L65; L72; L66; L47 |
| 559 | YIFFASFYY | L12; L1; L58; L26; L14; | TILDGISQY | L14; L11; L10; L26; L6; L16; L43; |
| | | L18; L6; L59; L69 | | L12; L27; L4; L18; L7; L17; L58; |
| | | | | L13; L31; L69; L29; L19; L59 |
| 560 | YIIKLIFL | L74; L58; L23; L59; L60; | TLDSKTQSL | L23; L5; L2; L63; L68; L32; L35; |
| | | L73; L61; L67; L69 | | L1; L62; L34; L57; L4; L43; L28; |
| | | | | L39; L3; L16; L49; L74; L10 |
| 561 | YLYALVYF | L74; L59; L62; L26; L43; | VMVELVAEL | L67; L5; L3; L4; L2; L64; L62; L69; |
| | | L69; L27; L12; L58 | | L56; L61; L72; L31; L60; L66; |
| | | | | L71; L8; L44; L9; L20; L73 |
| 562 | YQDVNCTEV | L24; L68; L63; L62; L28; | AAYVDNSSL | L43; L61; L67; L68; L70; L60; L59; |
| | | L48; L35; L58; L5 | | L69; L58; L57; L53; L63; L32; |
| | | | | L44; L56; L72; L31; L74; L54; L26; |
| | | | | L28 |
| 563 | YRVVVLSF | L66; L65; L35; L25; L71; | EYHDVRVVL | L66; L8; L71; L72; L9; L35; L20; |
| | | L59; L72; L64; L30 | | L32; L62; L44; L67; L34; L25; L17; |
| | | | | L41; L65; L56; L39; L13; L23; L64 |
| 564 | YSTAALGVL | L61; L60; L59; L73; L58; | FAIGLALYY | L69; L59; L12; L70; L31; L58; L67; |
| | | L70; L74; L67; L69 | | L74; L64; L61; L49; L60; L43; |
| | | | | L14; L11; L1; L18; L52; L32; L73; |
| | | | | L55 |
| 565 | YTSNPTTF | L58; L70; L60; L54; L59; | HVGEIPVAY | L43; L12; L26; L11; L14; L31; L6; |
| | | L53; L62; L69; L55 | | L49; L29; L10; L16; L20; L58; L13; |
| | | | | L66; L54; L18; L52; L69; L65; |
| | | | | L44 |
| 566 | YYQLYSTQL | L71; L72; L9; L66; L62; | KAYNVTQAF | L59; L43; L54; L58; L53; L69; L70; |
| | | L8; L28; L65; L60 | | L49; L52; L16; L27; L60; L61; |
| | | | | L26; L55; L31; L12; L56; L72; L71; |
| | | | | L14 |
| 567 | YYRSLPGVF | L66; L9; L8; L72; L62; | KLFDRYFKY | L16; L12; L6; L14; L26; L18; L27; |
| | | L71; L12; L58; L27 | | L43; L69; L13; L11; L4; L10; L54; |
| | | | | L21; L2; L24; L5; L55; L66; L3 |
| 568 | AALALLLL | L74; L73; L59; L61; L47; | KSAPLIEL | L73; L59; L55; L74; L69; L70; L53; |
| | | L67; L60; L23; L69; L70 | | L67; L54; L61; L60; L58; L65; |
| | | | | L64; L66; L56; L43; L26; L33; L18; |
| | | | | L44 |
| 569 | AEIPKEEV | L45; L37; L33; L38; L46; | NSFSGYLKL | L25; L73; L70; L69; L55; L35; L74; |
| | | L42; L36; L29; L48; L23 | | L67; L60; L10; L53; L61; L16; |
| | | | | L49; L58; L64; L11; L20; L54; L13; |
| | | | | L24 |
| 570 | AEWFLAYIL | L37; L36; L40; L42; L41; | SLDNVLSTF | L5; L62; L68; L63; L26; L34; L10; |
| | | L44; L45; L33; L38; L29 | | L43; L9; L27; L12; L2; L53; L1; |
| | | | | L49; L16; L18; L74; L8; L3; |
| 571 | AFPFTIYSL | L72; L71; L62; L57; L8; | STVFPPTSF | L43; L60; L53; L52; L58; L16; L12; |
| | | L9; L66; L65; L5; L44 | | L10; L54; L26; L11; L70; L7; L28; |
| | | | | L27; L20; L69; L59; L61; L9; L65 |
| 572 | AIFYLITPV | L24; L3; L4; L69; L20; L2; | TMADLVYAL | L4; L2; L35; L67; L3; L20; L34; |
| | | L5; L7; L6; L13 | | L44; L64; L56; L28; L32; L16; L31; |
| | | | | L5; L66; L65; L40; L69; L8; L36 |
| 573 | AVFDKNLY | L14; L18; L26; L12; L1; | TVFPPTSF | L26; L43; L29; L58; L16; L54; L59; |
| | | L65; L16; L58; L54; L6 | | L74; L52; L23; L28; L65; L69; |
| | | | | L55; L25; L60; L31; L62; L53; L12; |
| | | | | L72 |
| 574 | AVKTQFNYY | L13; L14; L26; L58; L12; | VMYASAVVL | L67; L25; L69; L28; L72; L71; L61; |
| | | L10; L6; L18; L11; L1 | | L35; L44; L31; L60; L66; L62; |
| | | | | L70; L56; L39; L68; L74; L47; L64; |
| | | | | L27 |
| 575 | AVTANVNAL | L56; L10; L61; L16; L60; | VVFLLVTL | L59; L67; L74; L23; L56; L25; L60; |
| | | L67; L4; L44; L3; L74 | | L48; L47; L26; L68; L39; L62; |
| | | | | L69; L44; L64; L20; L55; L53; L63; |
| | | | | L33 |
| 576 | CTDDNALAY | L1; L18; L12; L14; L68; | YASAVVLL | L74; L69; L70; L67; L64; L58; L61; |
| | | L59; L11; L58; L10; L16 | | L60; L47; L63; L31; L59; L73; |
| | | | | L62; L56; L68; L49; L23; L32; L66; |
| | | | | L20 |
| 577 | CYFPLQSY | L71; L72; L66; L59; L12; | GVYSVIYLY | L12; L14; L6; L7; L11; L26; L58; |
| | | L29; L26; L14; L64; L43 | | L18; L24; L27; L16; L13; L10; L49; |
| | | | | L55; L54; L52; L1; L65; L19; L53; |
| | | | | L69 |
| 578 | DFYLCFLAF | L25; L72; L9; L71; L29; | ILFLALITL | L2; L4; L3; L5; L26; L64; L68; L74; |
| | | L59; L47; L12; L8; L11 | | L59; L69; L23; L6; L56; L67; L16; |
| | | | | L44; L62; L27; L53; L47; L57; L61 |
| 579 | DTYPSLETI | L47; L10; L20; L25; L53; | KAYKIEEL | L74; L67; L59; L70; L58; L55; L54; |
| | | L11; L16; L52; L34; L49 | | L69; L73; L23; L53; L61; L63; |
| | | | | L60; L66; L68; L43; L65; L27; L44; |
| | | | | L25; L56 |
| 580 | EAIRHVRAW | L10; L49; L52; L54; L11; | LAFLLFLVL | L67; L59; L61; L47; L60; L69; L31; |
| | | L61; L69; L17; L40; L32 | | L70; L73; L50; L74; L32; L68; |
| | | | | L64; L58; L56; L44; L55; L25; L49; |
| | | | | L52; L53 |
| 581 | EHFIETISL | L34; L28; L35; L25; L23; | LANECAQVL | L32; L61; L60; L70; L59; L31; L69; |
| | | L20; L44; L27; L10; L41 | | L58; L67; L55; L64; L68; L57; |
| | | | | L47; L63; L49; L39; L73; L53; L56; |
| | | | | L43; L23 |
| 582 | EIVDTVSAL | L10; L11; L20; L67; L61; | LLADKFPVL | L2; L4; L5; L3; L61; L59; L69; L74; |
| | | L28; L60; L35; L19; L32 | | L9; L39; L23; L44; L8; L43; L56; |
| | | | | L67; L66; L28; L20; L6; L64; L16 |
| 583 | ELDERIDKV | L5; L2; L24; L1; L11; L23; | SAPPAQYEL | L57; L67; L61; L59; L66; L60; L58; |
| | | L4; L20; L63; L3 | | L64; L5; L65; L69; L74; L70; L63; |
| | | | | L32; L68; L53; L56; L20; L39; L62 |
| | | | | L43 |
| 584 | FAQDGNAAI | L61; L59; L67; L68; L49; | SASKIITL | L74; L70; L58; L69; L23; L56; L61; |
| | | L32; L70; L43; L50; L20 | | L32; L67; L60; L64; L39; L59; |
| | | | | L44; L53; L63; L73; L68; L49; L47; |
| | | | | L35; L66 |
| 585 | FATSACVL | L61; L74; L67; L70; L59; | SHFAIGLAL | L35; L28; L34; L25; L44; L56; L61; |
| | | L60; L69; L58; L55; L68 | | L20; L60; L71; L27; L72; L31; |
| | | | | L22; L41; L37; L39; L32; L64; L67; |
| | | | | L40; L36 |
| 586 | FAYANRNRFL | L60; L61; L58; L69; L70; | SSSDNIALL | L73; L74; L20; L58; L64; L69; L70; |
| | | L74; L64; L67; L68; L32 | | L53; L34; L61; L60; L44; L4; L65; |
| | | | | L1; L63; L56; L19; L35; L3; L28; |
| | | | | L68 |
| 587 | FCNDPFLGV | L73; L5; L59; L50; L58; | SVIYLYLTF | L10; L11; L12; L49; L58; L26; L16; |
| | | L61; L4; L3; L2; L47 | | L52; L43; L53; L7; L8; L9; L34; |
| | | | | L27; L59; L54; L73; L60; L41; L69; |
| | | | | L32 |
| 588 | FFITGNTL | L72; L71; L62; L61; L59; | TLKEILVTY | L43; L26; L3; L14; L13; L12; L10; |
| | | L60; L67; L9; L8; L68 | | L11; L27; L16; L69; L6; L4; L2; |
| | | | | L23; L5; L49; L18; L58; L55; L17; |
| | | | | L15 |
| 589 | FLAFLLFL | L74; L59; L2; L3; L4; L61; | VAFNTLLFL | L69; L74; L60; L58; L59; L61; L70; |
| | | L64; L68; L23; L1 | | L67; L47; L73; L64; L31; L68; |
| | | | | L53; L4; L20; L44; L63; L56; L57; |
| | | | | L49; L32 |
| 590 | FLHFLPRVF | L69; L59; L64; L43; L26; | YADVFHLYL | L67; L68; L74; L59; L61; L60; L63; |
| | | L12; L62; L27; L74; L66 | | L32; L70; L62; L1; L73; L58; L69; |
| | | | | L5; L49; L31; L64; L57; L18; L47; |
| | | | | L34 |
| 591 | FLMSFTVL | L74; L59; L61; L57; L23; | YAFEHIVY | L59; L70; L69; L31; L58; L27; L66; |
| | | L68; L67; L2; L62; L72 | | L67; L26; L29; L47; L43; L61; |
| | | | | L49; L54; L50; L12; L72; L71; L64; |
| | | | | L60; L25 |
| 592 | FSSEIIGY | L1; L58; L69; L70; L43; | ANDPVGFTL | L68; L34; L35; L5; L63; L28; L67; |
| | | L18; L59; L26; L66; L55 | | L65; L61; L60; L62; L55; L39; L36; |
| | | | | L32; L66; L53; L44; L41; L22; |
| | | | | L54; L8; L73 |
| 593 | FTSDYYQL | L67; L60; L70; L61; L59; | FVNEFYAYL | L60; L5; L61; L58; L59; L74; L67; |
| | | L66; L20; L58; L73; L55 | | L64; L62; L73; L20; L4; L68; L10; |
| | | | | L11; L3; L69; L2; L57; L43; L63; |
| | | | | L66; L32 |
| 594 | FVAAIFYL | L74; L20; L58; L60; L61; | FVSDADSTL | L60; L61; L67; L58; L32; L20; L68; |
| | | L64; L69; L59; L67; L4 | | L74; L5; L57; L63; L59; L2; L28; |
| | | | | L4; L34; L69; L66; L3; L10; L44; |
| | | | | L64; L31 |
| 595 | FVCNLLLL | L74; L59; L67; L60; L73; | HAASGNLLL | L74; L32; L44; L61; L70; L67; L69; |
| | | L61; L68; L64; L62; L58 | | L58; L73; L49; L64; L31; L60; |
| | | | | L53; L20; L34; L56; L54; L35; L63; |
| | | | | L68; L11; L28 |
| 596 | FVFPLNSII | L69; L20; L64; L67; L47; | IVAGGIVAI | L20; L3; L10; L24; L49; L11; L67; |
| | | L3; L16; L74; L61; L32 | | L39; L4; L16; L64; L28; L53; L34; |
| | | | | L32; L19; L51; L6; L68; L74; L2; |
| | | | | L69; L26 |
| 597 | FVLTSHTVM | L61; L59; L71; L60; L32; | IVNSVLLFL | L73; L74; L4; L5; L58; L60; L6; |
| | | L31; L74; L72; L67; L58 | | L64; L56; L61; L20; L59; L53; L68; |
| | | | | L39; L67; L31; L3; L62; L63; L32; |
| | | | | L8; L69 |
| 598 | GAAAYYVGY | L43; L58; L12; L27; L31; | SLIDLQEL | L4; L59; L2; L3; L23; L43; L24; |
| | | L49; L18; L14; L10; L26 | | L74; L5; L64; L26; L67; L61; L57; |
| | | | | L72; L25; L56; L68; L10; L71; L69; |
| | | | | L37; L20 |
| 599 | GLNDNLLEI | L3; L24; L4; L2; L5; L16; | VLNDILSRL | L4; L5; L3; L2; L43; L8; L16; L64; |
| | | L10; L73; L6; L8 | | L26; L62; L44; L74; L57; L63; L6; |
| | | | | L56; L24; L73; L9; L69; L11; L14; |
| | | | | L10 |
| 600 | GRVDGQVDL | L30; L35; L65; L64; L66; | VVIPDYNTY | L43; L11; L26; L10; L14; L52; L12; |
| | | L34; L44; L25; L28; L36 | | L54; L58; L59; L16; L49; L18; |
| | | | | L27; L53; L31; L13; L6; L29; L55; |
| | | | | L69; L7; L1 |
| 601 | GSLPINVIVF | L52; L53; L55; L54; L26; | YFFTLLLQL | L71; L72; L64; L69; L9; L8; L4; |
| | | L27; L69; L65; L16; L44 | | L62; L73; L59; L3; L67; L44; L31; |
| | | | | L61; L60; L34; L12; L58; L35; L25; |
| | | | | L20; L74 |
| 602 | GVYDYLVST | L6; L7; L43; L3; L24; L2; | YVYSRVKNL | L74; L60; L64; L61; L58; L69; L23; |
| | | L4; L10; L15; L44 | | L25; L3; L10; L4; L73; L39; L67; |
| | | | | L49; L20; L11; L5; L59; L32; L70; |
| | | | | L47; L16 |
| 603 | HAEETRKL | L63; L32; L49; L60; L68; | DADSKIVQL | L32; L74; L63; L23; L49; L68; L60; |
| | | L70; L61; L55; L23; L62 | | L70; L67; L35; L58; L69; L31; |
| | | | | L61; L34; L20; L47; L10; L62; L11; |
| | | | | L5; L17; L19; L53 |
| 604 | HAFLCLFL | L74; L69; L59; L70; L60; | IIFWFSLEL | L59; L60; L73; L61; L65; L67; L58; |
| | | L47; L61; L67; L73; L25 | | L64; L69; L43; L5; L68; L16; L74; |
| | | | | L3; L26; L6; L4; L32; L57; L28; |
| | | | | L2; L20; L72 |
| 605 | HFAIGLAL | L72; L71; L25; L35; L23; | KSFDLGDEL | L55; L67; L53; L73; L54; L44; L69; |
| | | L28; L61; L59; L62; L67 | | L16; L70; L58; L13; L60; L61; |
| | | | | L66; L56; L20; L65; L52; L14; L27; |
| | | | | L64; L26; L59; L18 |
| 606 | HVVAFNTL | L60; L20; L67; L25; L56; | QSASKIITL | L70; L69; L73; L55; L53; L20; L74; |
| | | L59; L61; L10; L23; L29 | | L44; L61; L64; L58; L56; L65; |
| | | | | L10; L60; L32; L23; L39; L43; L34; |
| | | | | L54; L67; L16; L35 |
| 607 | IAGLIAIVM | L70; L67; L69; L31; L58; | SAFVNLKQL | L69; L59; L60; L70; L58; L64; L61; |
| | | L68; L32; L59; L61; L47 | | L56; L74; L32; L25; L44; L10; |
| | | | | L73; L31; L39; L3; L49; L4; L13; |
| | | | | L47; L20; L57; L53 |
| 608 | IEDLLFNKV | L45; L46; L29; L37; L38; | SAPHGVVFL | L74; L57; L63; L69; L64; L67; L58; |
| | | L42; L36; L33; L5; L62 | | L68; L61; L70; L32; L56; L49; |
| | | | | L20; L66; L62; L60; L5; L59; L72; |
| | | | | L31; L44; L47; L73 |
| 609 | ILDITPCSF | L62; L68; L63; L43; L5; | SAQTGIAVL | L61; L32; L60; L70; L69; L56; L28; |
| | | L18; L1; L74; L9; L26 | | L58; L68; L74; L65; L59; L63; |
| | | | | L44; L67; L39; L10; L31; L72; L57; |
| | | | | L64; L53; L66; L49 |
| 610 | ISAGFSLWVY | L18; L14; L1; L43; L26; | SLLMPILTL | L4; L2; L5; L6; L16; L3; L61; L23; |
| | | L52; L58; L69; L53; L54 | | L44; L74; L24; L27; L8; L73; L9; |
| | | | | L34; L26; L10; L67; L59; L56; L28; |
| | | | | L13; L39 |
| 611 | ITVATSRTL | L55; L60; L61; L53; L70; | VADAVIKTL | L60; L68; L63; L32; L67; L70; L61; |
| | | L54; L65; L10; L64; L73 | | L5; L74; L58; L69; L62; L39; L49; |
| | | | | L47; L31; L44; L55; L53; L56; L64; |
| | | | | L73; L43; L59 |
| 612 | IVAAIVFITL | L20; L56; L67; L31; L60; | YVFCTVNAL | L59; L60; L61; L67; L32; L64; L4; |
| | | L10; L39; L5; L6; L53 | | L20; L10; L58; L74; L3; L69; L71; |
| | | | | L5; L72; L44; L68; L28; L16; L31; |
| | | | | L65; L57; L26 |
| 613 | IVDTVSAL | L68; L63; L62; L67; L74; | AGIVGVLTL | L61; L73; L69; L34; L44; L67; L35; |
| | | L60; L28; L61; L5; L1 | | L72; L71; L28; L27; L16; L59; |
| | | | | L24; L8; L26; L3; L65; L64; L25; L4; |
| | | | | L43; L37; L60; L55 |
| 614 | KFKEGVEFL | L13; L72; L8; L62; L9; | AMYTPHTVL | L25; L61; L28; L67; L44; L56; L27; |
| | | L71; L3; L55; L16; L44 | | L68; L69; L71; L72; L35; L26; |
| | | | | L66; L36; L60; L70; L39; L65; L59; |
| | | | | L22; L16; L3; L8; L74 |
| 615 | KFLPFQQF | L8; L9; L71; L33; L72; | AQLPAPRTL | L44; L55; L61; L27; L73; L4; L26; |
| | | L54; L55; L27; L53; L52 | | L16; L64; L28; L35; L69; L24; L48; |
| | | | | L25; L60; L34; L3; L40; L30; L13; |
| | | | | L66; L41; L8; L67 |
| 616 | KHADFDTWF | L34; L53; L54; L28; L27; | FAFPFTIYSL | L67; L61; L32; L60; L59; L69; L70; |
| | | L16; L35; L26; L8; L9 | | L31; L20; L58; L74; L3; L50; L64; |
| | | | | L43; L66; L47; L65; L44; L4; L49; |
| | | | | L68; L53; L10; L5 |
| 617 | KLHDELTGH | L3; L6; L2; L4; L26; L24; | GVAPGTAVL | L28; L44; L20; L74; L10; L3; L67; |
| | | L21; L43; L16; L5 | | L56; L61; L26; L22; L58; L60; L11; |
| | | | | L16; L35; L59; L53; L4; L64; L39; |
| | | | | L2; L54; L36; L34 |
| 618 | KLIFLWLLW | L16; L52; L54; L12; L6; | HLDGEVITF | L34; L63; L5; L49; L62; L32; L68; |
| | | L24; L18; L9; L73; L21 | | L26; L53; L2; L27; L65; L74; L10; |
| | | | | L18; L16; L35; L54; L24; L12; L9; |
| | | | | L28; L11; L1; L31 |
| 619 | KLINIIIWFL | L4; L3; L5; L2; L16; L44; | KLINIIIWF | L16; L26; L69; L43; L5; L53; L8; |
| | | L6; L20; L13; L24 | | L27; L4; L11; L6; L13; L9; L52; L74; |
| | | | | L12; L24; L54; L21; L3; L2; L64; |
| | | | | L10; L44; L55 |
| 620 | KLNIKLLGV | L3; L73; L4; L2; L5; L23; | KVDGVDVEL | L5; L63; L68; L67; L74; L62; L32; |
| | | L24; L18; L6; L16 | | L56; L53; L54; L60; L2; L39; L73; |
| | | | | L44; L18; L58; L20; L16; L65; L49; |
| | | | | L13; L55; L34; L61 |
| 621 | KLSYGIATV | L3; L2; L4; L24; L5; L16; | QINDMILSL | L73; L4; L65; L43; L16; L61; L5; |
| | | L18; L73; L20; L21 | | L69; L3; L10; L23; L60; L39; L64; |
| | | | | L68; L59; L58; L66; L32; L11; L6; |
| | | | | L35; L57; L72; L67 |
| 622 | KSYELQTPF | L53; L54; L55; L52; L16; | SALEPLVDL | L60; L67; L61; L59; L31; L74; L56; |
| | | L43; L27; L26; L73; L58 | | L68; L4; L44; L58; L69; L53; L63; |
| | | | | L32; L39; L23; L70; L73; L43; L2; |
| | | | | L50; L49; L5; L47 |
| 623 | KVATVQSKM | L54; L14; L53; L58; L6; | YQPYRVVVL | L44; L64; L57; L69; L66; L23; L27; |
| | | L13; L18; L52; L16; L11 | | L65; L67; L25; L71; L28; L58; |
| | | | | L35; L72; L61; L26; L5; L62; L33; |
| | | | | L48; L63; L39; L24; L73 |
| 624 | LAKDTTEAF | L43; L58; L59; L49; L70; | AVASKILGL | L3; L10; L4; L11; L16; L6; L73; |
| | | L52; L31; L26; L60; L32 | | L20; L7; L74; L56; L58; L69; L24; |
| | | | | L39; L44; L23; L61; L13; L2; L60; L5; |
| | | | | L64; L22; L43; L26 |
| 625 | LAMDEFIER | L17; L59; L15; L19; L31; | EAFEKMVSL | L23; L32; L20; L69; L10; L61; L60; |
| | | L7; L61; L21; L60; L69 | | L70; L67; L31; L25; L43; L44; |
| | | | | L59; L11; L19; L35; L58; L49; L56; |
| | | | | L64; L74; L47; L17; L63; L50 |
| 626 | LATNNLVVM | L70; L59; L69; L58; L61; | FVVFLLVTL | L67; L60; L20; L74; L61; L59; L69; |
| | | L31; L32; L60; L50; L47 | | L58; L5; L64; L31; L32; L68; L73; |
| | | | | L50; L4; L44; L3; L56; L49; L10; |
| | | | | L34; L2; L19; L53; L26 |
| 627 | LNNDYYRSL | L43; L65; L32; L73; L64; | HSSGVTREL | L65; L60; L55; L73; L70; L64; L32; |
| | | L55; L61; L66; L39; L60 | | L61; L53; L44; L58; L49; L59; |
| | | | | L69; L67; L57; L20; L39; L54; L35; |
| | | | | L56; L66; L34; L43; L24; L74 |
| 628 | LPFFSNVTW | L49; L31; L50; L47; L32; | LMIERFVSL | L61; L23; L60; L4; L5; L67; L25; |
| | | L56; L52; L39; L22; L51 | | L3; L59; L69; L32; L43; L73; L8; L2; |
| | | | | L72; L39; L71; L56; L44; L26; L31; |
| | | | | L9; L28; L74; L57 |
| 629 | LPVNVAFEL | L32; L56; L31; L39; L47; | TVASLINTL | L20; L4; L10; L3; L64; L34; L16; |
| | | L50; L22; L49; L65; L61 | | L35; L28; L32; L56; L44; L74; L69; |
| | | | | L19; L67; L39; L11; L53; L49; L58; |
| | | | | L31; L26; L55; L24; L2 |
| 630 | LVDSDLNDF | L1; L68; L49; L63; L5; | HVISTSHKL | L11; L32; L10; L24; L20; L44; L60; |
| | | L53; L62; L58; L31; L52 | | L54; L49; L58; L53; L8; L63; L28; |
| | | | | L34; L35; L73; L74; L61; L16; L13; |
| | | | | L14; L37; L67; L5; L55; L39 |
| 631 | LVQAGNVQL | L56; L32; L39; L60; L4; | ITFDNLKTL | L70; L60; L69; L53; L64; L58; L10; |
| | | L28; L20; L31; L61; L74 | | L55; L61; L73; L20; L16; L59; L3; |
| | | | | L4; L52; L11; L13; L26; L39; L25; |
| | | | | L54; L67; L8; L5; L2; L37 |
| 632 | LYDKLVSSF | L62; L9; L8; L72; L63; | SFYEDFLEY | L71; L12; L72; L27; L14; L13; L59; |
| | | L71; L68; L49; L12; L66 | | L43; L49; L26; L6; L31; L65; L10; |
| | | | | L11; L8; L16; L66; L18; L29; L69; |
| | | | | L58; L7; L1; L9; L67; L41 |
| 633 | MLFTMLRKL | L64; L3; L4; L10; L69; L2; | YLATALLTL | L3; L4; L2; L74; L10; L61; L5; L59; |
| | | L16; L5; L74; L24 | | L44; L64; L69; L67; L57; L73; L9; |
| | | | | L28; L16; L23; L68; L1; L24; L34; |
| | | | | L66; L56; L26; L60; L8 |
| 634 | MLIIFWFSL | L4; L23; L5; L43; L2; L16; | VAAIVFITL | L67; L61; L60; L59; L74; L70; L69; |
| | | L69; L61; L20; L26 | | L68; L53; L47; L32; L58; L31; |
| | | | | L56; L20; L73; L44; L49; L39; L43; |
| | | | | L64; L9; L23; L66; L55; L26; L52; |
| | | | | L50; L54 |
| 635 | MVDTSLSGF | L10; L63; L11; L1; L68; | KVDGVVQQL | L5; L63; L68; L2; L74; L54; L53; |
| | | L62; L58; L18; L5; L49 | | L13; L16; L67; L73; L39; L60; L24; |
| | | | | L62; L55; L56; L58; L34; L64; L65; |
| | | | | L44; L49; L18; L32; L20; L4; L3; |
| | | | | L14; L26 |
| 636 | NASSSEAFL | L74; L67; L70; L58; L31; | ATIPIQASL | L61; L20; L67; L53; L54; L16; L58; |
| | | L20; L32; L60; L57; L61 | | L73; L43; L60; L28; L70; L10; |
| | | | | L69; L55; L44; L52; L4; L11; L13; L3; |
| | | | | L40; L59; L36; L5; L14; L26; L35; |
| | | | | L37; L22; L7; L6; L41 |
| 637 | NELSRVLGL | L25; L33; L40; L41; L37; | YITGGVVQL | L74; L60; L2; L61; L58; L4; L67; |
| | | L23; L45; L36; L29; L8 | | L3; L69; L44; L20; L64; L73; L28; |
| | | | | L63; L56; L68; L32; L59; L10; L34; |
| | | | | L5; L35; L49; L11; L26; L39; L65; |
| | | | | L66; L70; L43; L19; L16; L24 |
| 638 | NFKDQVILL | L62; L13; L8; L72; L71; | FGADPIHSL | L61; L60; L67; L59; L44; L69; L64; |
| | | L64; L9; L69; L70; L66 | | L43; L4; L31; L65; L66; L68; L32; |
| | | | | L35; L28; L20; L2; L70; L34; L58; |
| | | | | L10; L3; L16; L39; L5; L11; L63; |
| | | | | L47; L23; L49; L72; L26; L57; L56 |

The viral genome comprises multiple genes encoded by multiple reading frames spanning a single polynucleotide stretch. For example, the nucleocapsid protein is an abundantly expressed protein in 2019 SARS CoV-2 virus. A short protein ORF9b is encoded by another reading frame spanning the region nucleocapsid sequence. These highly expressed proteins expand the number of potential targets for T cell immunity. Table 1C and Table 2B shows predicted MHC-I binding epitopes and MHC-II binding epitopes from Orf9b respectively.

TABLE 1C
MHC I-binding Epitopes from Orf9b

| Set 1 Peptides | Set 1 Alleles | Set 2 Peptides | Set 2 Alleles |
|---|---|---|---|
| ALRLVDPQI | HLA-A02:04 | RMENAVGR | HLA-A31:01; HLA-A74:01 |
| EELPDEFV | HLA-B18:01 | SPLSLNMA | HLA-B54:01; HLA-B55:01 |
| ELPDEFVV | HLA-A02:07 | TKLATTEEL | HLA-B48:01; HLA-B15:03 |
| EMHPALRL | HLA-B52:01 | TPIAVQMTK | HLA-B35:03; HLA-A68:01 |
| GPKVYPII | HLA-B51:01 | VDPQIQLAV | HLA-C01:02; HLA-A02:07 |
| GPKVYPIILR | HLA-A31:01 | VTRMENAVGR | HLA-A74:01; HLA-A31:01 |
| GRDQNNVGPK | HLA-B27:05 | DEFVVVTVK | HLA-B18:01; HLA-B14:02; |
| | | | HLA-A68:01 |
| GRDQNNVGPKV | HLA-B27:05 | FQLTPIAV | HLA-B52:01; HLA-B54:01; |
| | | | HLA-B14:02 |
| ILRLGSPL | HLA-C01:02 | HPALRLVDP | HLA-B55:01; HLA-B08:01; |
| | | | HLA-B54:01 |
| IQLAVTRM | HLA-B52:01 | ISEMHPALRL | HLA-C05:01; HLA-A36:01; |
| | | | HLA-A01:01 |
| ISEMHPALR | HLA-A01:01 | KLATTEEL | HLA-C17:01; HLA-A02:01; |
| | | | HLA-A02:03 |
| KTLNSLED | HLA-B58:02 | KTLNSLEDK | HLA-A03:01; HLA-A11:01; |
| | | | HLA-A30:01 |
| KTLNSLEDKAF | HLA-B57:01 | LEDKAFQLTP | HLA-B41:01; HLA-B50:01; |
| | | | HLA-B45:01 |
| LATTEELPDEF | HLA-B57:01 | LVDPQIQLAV | HLA-A02:07; HLA-C08:02; |
| | | | HLA-A01:01 |
| LEDKAFQLT | HLA-B49:01 | MHPALRLV | HLA-C06:02; HLA-B51:01; |
| | | | HLA-B52:01 |
| LNMARKTL | HLA-B14:02 | PQIQLAVTR | HLA-A74:01; HLA-A31:01; |
| | | | HLA-A68:01 |
| LPDEFVVVTVK | HLA-B35:01 | RLVDPQIQLA | HLA-A02:01; HLA-B13:02; |
| | | | HLA-A02:03 |
| LRLGSPLSLN | HLA-B27:05 | SPLSLNMAR | HLA-A33:03; HLA-A31:01; |
| | | | HLA-A74:01 |
| LRLGSPLSLNM | HLA-B27:05 | TEELPDEF | HLA-B18:01; HLA-B37:01; |
| | | | HLA-C04:01 |
| LRLVDPQIQ | HLA-B27:05 | VGPKVYPIIL | HLA-C01:02; HLA-B48:01; |
| | | | HLA-C07:02 |
| LRLVDPQIQL | HLA-B27:05 | VYPIILRLG | HLA-A24:02; HLA-C06:02; |
| | | | HLA-A23:01 |
| LSLNMARKT | HLA-B58:02 | AFQLTPIAV | HLA-C14:03; HLA-C04:01; |
| | | | HLA-C14:02; HLA-B41:01 |
| NAVGRDQNN | HLA-B53:01 | ATTEELPDEF | HLA-B57:01; HLA-B58:01; |
| | | | HLA-B57:03; HLA-A29:02 |
| NMARKTLNSL | HLA-A25:01 | DPQIQLAV | HLA-B51:01; HLA-B08:01; |
| | | | HLA-B14:02; HLA-B54:01 |
| NNVGPKVY | HLA-B18:01 | EMHPALRLV | HLA-B52:01; HLA-C06:02; |
| | | | HLA-A02:03; HLA-A68:02 |
| NNVGPKVYP | HLA-C07:01 | GPKVYPIIL | HLA-B42:01; HLA-B81:01; |
| | | | HLA-B07:02; HLA-B08:01 |
| NSLEDKAF | HLA-B58:02 | HPALRLVD | HLA-B08:01; HLA-B55:01; |
| | | | HLA-B14:02; HLA-B54:01 |
| NVGPKVYPI | HLA-A68:02 | KVYPIILRLG | HLA-B57:01; HLA-A30:02; |
| | | | HLA-A03:01; HLA-A32:01 |
| NVGPKVYPIIL | HLA-C08:01 | TEELPDEFV | HLA-B41:01; HLA-B40:01; |
| | | | HLA-B49:01; HLA-B45:01 |
| NVGPKVYPIILR | HLA-A68:01 | TEELPDEFVV | HLA-B41:01; HLA-B40:01; |
| | | | HLA-B45:01; HLA-B49:01 |
| PKVYPIILR | HLA-A68:01 | TEELPDEFVVV | HLA-B40:01; HLA-B41:01; |
| | | | HLA-B45:01; HLA-B49:01 |
| QIQLAVTR | HLA-A74:01 | ARKTLNSL | HLA-C07:02; HLA-B14:02; |
| | | | HLA-B08:01; HLA-C06:02; |
| | | | HLA-B39:01 |
| QNNVGPKV | HLA-B52:01 | ELPDEFVVVTV | HLA-A26:01; HLA-A02:07; |
| | | | HLA-A68:02; HLA-A02:01; |
| | | | HLA-A02:03 |
| QNNVGPKVY | HLA-B18:01 | KAFQLTPIAV | HLA-C12:02; HLA-B54:01; |
| | | | HLA-C12:03; HLA-A02:03; |
| | | | HLA-A68:02 |
| RDQNNVGP | HLA-B37:01 | LEDKAFQL | HLA-B37:01; HLA-B40:02; |
| | | | HLA-C05:01; HLA-B49:01; |
| | | | HLA-B18:01 |
| RDQNNVGPKVY | HLA-A30:02 | LVDPQIQLA | HLA-A02:07; HLA-A01:01; |
| | | | HLA-A36:01; HLA-C05:01; |
| | | | HLA-C08:02 |
| RKTLNSLED | HLA-B15:03 | TPIAVQMTKL | HLA-B35:03; HLA-B81:01; |
| | | | HLA-B07:02; HLA-B35:01; |
| | | | HLA-B42:01 |
| RLGSPLSLN | HLA-A03:01 | TRMENAVGR | HLA-B27:05; HLA-A68:01; |
| | | | HLA-A33:03; HLA-A31:01; |
| | | | HLA-A74:01 |
| RLGSPLSLNM | HLA-A03:01 | ISEMHPAL | HLA-C08:02; HLA-C05:01; |
| | | | HLA-C08:01; HLA-C03:03; |
| | | | HLA-C03:04; HLA-C03:02 |
| TTEELPDEFV | HLA-A68:02 | SEMHPALRLV | HLA-B49:01; HLA-B45:01; |
| | | | HLA-B41:01; HLA-B44:02; |
| | | | HLA-B40:02; HLA-B44:03 |
| VGPKVYPII | HLA-B51:01 | SLNMARKTL | HLA-C01:02; HLA-A02:04; |
| | | | HLA-A32:01; HLA-B42:01; |
| | | | HLA-B08:01; HLA-A02:03 |
| VGRDQNNV | HLA-B52:01 | KVYPIILR | HLA-A74:01; HLA-A31:01; |
| | | | HLA-A03:01; HLA-A11:01; |
| | | | HLA-A33:03; HLA-A68:01; |
| | | | HLA-A30:01 |
| YPIILRLG | HLA-B51:01 | MARKTLNSL | HLA-C03:02; HLA-B35:03; |
| | | | HLA-C02:02; HLA-C17:01; |
| | | | HLA-C12:03; HLA-C03:03; |
| | | | HLA-C01:02 |
| YPIILRLGS | HLA-B54:01 | SLEDKAFQL | HLA-A02:04; HLA-A02:01; |
| | | | HLA-A02:07; HLA-C05:01; |
| | | | HLA-B08:01; HLA-B42:01; |
| | | | HLA-C08:02 |
| YPIILRLGSP | HLA-B54:01 | DEFVVVTV | HLA-B18:01; HLA-B52:01; |
| | | | HLA-B14:02; HLA-B51:01; |
| | | | HLA-B49:01; HLA-B37:01; |
| | | | HLA-B40:02; HLA-B08:01 |
| YPIILRLGSPL | HLA-B07:02 | EELPDEFVV | HLA-B45:01; HLA-B40:02; |
| | | | HLA-B44:02; HLA-B18:01; |
| | | | HLA-B41:01; HLA-B49:01; |
| | | | HLA-B44:03; HLA-B40:01 |
| DQNNVGPKV | HLA-B13:02; | IAVQMTKL | HLA-C17:01; HLA-C03:03; |
| | HLA-B52:01 | | HLA-C03:04; HLA-C05:01; |
| | | | HLA-C12:03; HLA-B51:01; |
| | | | HLA-C08:01; HLA-C03:02 |
| DQNNVGPKVY | HLA-B15:01; | LPDEFVVVT | HLA-B35:01; HLA-B35:03; |
| | HLA-A30:02 | | HLA-B54:01; HLA-B53:01; |
| | | | HLA-B51:01; HLA-B42:01; |
| | | | HLA-B81:01; HLA-B55:01 |
| EDKAFQLTP | HLA-B45:01; | LPDEFVVVTV | HLA-B54:01; HLA-B51:01; |
| | HLA-B50:01 | | HLA-B35:03; HLA-A02:07; |
| | | | HLA-B35:01; HLA-B42:01; |
| | | | HLA-B53:01; HLA-B55:01 |
| EELPDEFVVVT | HLA-B44:03; | EELPDEFVVV | HLA-B45:01; HLA-B44:02; |
| | HLA-B44:02 | | HLA-B50:01; HLA-B40:02; |
| | | | HLA-B44:03; HLA-B49:01; |
| | | | HLA-B18:01; HLA-B41:01; |
| | | | HLA-B40:01 |
| GPKVYPIILRL | HLA-B81:01; | RLGSPLSL | HLA-C17:01; HLA-C15:02; |
| | HLA-B42:01 | | HLA-B58:02; HLA-C01:02; |
| | | | HLA-A02:03; HLA-A32:01; |
| | | | HLA-B81:01; HLA-B48:01; |
| | | | HLA-A02:04 |
| GRDQNNVGP | HLA-B39:01; | SEMHPALRL | HLA-B40:02; HLA-B40:01; |
| | HLA-B27:05 | | HLA-B49:01; HLA-B44:03; |
| | | | HLA-B44:02; HLA-B18:01; |
| | | | HLA-B37:01; HLA-B45:01; |
| | | | HLA-B41:01 |
| IILRLGSPL | HLA-C03:03; | ELPDEFVVV | HLA-A02:07; HLA-A68:02; |
| | HLA-C03:04 | | HLA-B13:02; HLA-A02:01; |
| | | | HLA-A26:01; HLA-C01:02; |
| | | | HLA-A02:03; HLA-A25:01; |
| | | | HLA-A02:04; HLA-C06:02 |
| ILRLGSPLSL | HLA-A02:03; | LVDPQIQL | HLA-C17:01; HLA-C05:01; |
| | HLA-B07:02 | | HLA-C08:02; HLA-A02:07; |
| | | | HLA-C08:01; HLA-A01:01; |
| | | | HLA-C03:03; HLA-C04:01; |
| | | | HLA-C03:04; HLA-B15:09 |
| KAFQLTPIA | HLA-C12:02; | TTEELPDEF | HLA-A01:01; HLA-C04:01; |
| | HLA-B54:01 | | HLA-B57:03; HLA-A29:02; |
| | | | HLA-B57:01; HLA-C05:01; |
| | | | HLA-A36:01; HLA-B35:01; |
| | | | HLA-B53:01; HLA-B38:01 |
| KISEMHPALR | HLA-A74:01; | VYPIILRL | HLA-C14:03; HLA-C04:01; |
| | HLA-A03:01 | | HLA-C07:02; HLA-A24:02; |
| | | | HLA-C14:02; HLA-A23:01; |
| | | | HLA-C06:02; HLA-B52:01; |
| | | | HLA-B51:01; HLA-C01:02 |
| KISEMHPALRL | HLA-C15:02; | LRLGSPLSL | HLA-B14:02; HLA-B39:01; |
| | HLA-A32:01 | | HLA-B27:05; HLA-C07:02; |
| | | | HLA-C07:01; HLA-C06:02; |
| | | | HLA-C03:04; HLA-B38:01; |
| | | | HLA-C03:03; HLA-B15:09; |
| | | | HLA-C08:01 |
| MDPKISEM | HLA-B37:01; | KISEMHPAL | HLA-A02:04; HLA-C08:01; |
| | HLA-C01:02 | | HLA-A32:01; HLA-A02:07; |
| | | | HLA-C15:02; HLA-C01:02; |
| | | | HLA-C03:03; HLA-C03:04; |
| | | | HLA-C07:02; HLA-A02:01; |
| | | | HLA-C03:02; HLA-B42:01 |
| NSLEDKAFQL | HLA-B58:02; | LPDEFVVV | HLA-B51:01; HLA-B54:01; |
| | HLA-B57:03 | | HLA-B42:01; HLA-C04:01; |
| | | | HLA-B55:01; HLA-B35:03; |
| | | | HLA-C08:02; HLA-C05:01; |
| | | | HLA-B08:01; HLA-A02:07; |
| | | | HLA-B53:01; HLA-B07:02 |
| QLTPIAVQM | HLA-A02:07; | RLVDPQIQL | HLA-A02:01; HLA-A02:07; |
| | HLA-A02:04 | | HLA-B48:01; HLA-A32:01; |
| | | | HLA-A30:01; HLA-A02:04; |
| | | | HLA-A03:01; HLA-B15:01; |
| | | | HLA-C07:01; HLA-B81:01; |
| | | | HLA-C17:01; HLA-B15:03; |
| | | | HLA-B40:01; HLA-B13:02; |
| | | | HLA-A23:01; HLA-A02:03; |
| | | | HLA-A68:02; HLA-B40:02; |
| | | | HLA-B57:03; HLA-B42:01; |
| | | | HLA-C15:02; HLA-C12:02; |
| | | | HLA-C03:04 |
| — | — | KVYPIILRL | HLA-A32:01; HLA-C06:02; |
| | | | HLA-C15:02; HLA-C12:02; |
| | | | HLA-A02:04; HLA-A68:02; |
| | | | HLA-C17:01; HLA-B13:02; |
| | | | HLA-A02:03; HLA-A02:07; |
| | | | HLA-B57:03; HLA-A03:01; |
| | | | HLA-C07:01; HLA-B58:01; |
| | | | HLA-C02:02; HLA-A26:01; |
| | | | HLA-C08:01; HLA-C03:03; |
| | | | HLA-A30:01; HLA-B57:01; |
| | | | HLA-A02:01; HLA-A29:02; |
| | | | HLA-B58:02; HLA-B48:01; |
| | | | HLA-C03:04; HLA-A23:01; |
| | | | HLA-B81:01; HLA-B46:01; |
| | | | HLA-B15:01; HLA-C12:03; |
| | | | HLA-B42:01; HLA-A25:01; |
| | | | HLA-A36:01; HLA-B53:01; |
| | | | HLA-A11:01; HLA-A30:02; |
| | | | HLA-C07:02; HLA-B15:03; |
| | | | HLA-B14:02; HLA-B55:01; |
| | | | HLA-C05:01; HLA-B40:02; |
| | | | HLA-B52:01 |

TABLE 1D
(Tables 1A-B Allele key)

| Allele | Alias | Allele | Alias | Allele | Alias | Allele | Alias |
|---|---|---|---|---|---|---|---|
| HLA-A01:01 | L1 | HLA-A68:01 | L19 | HLA-B40:02 | L37 | HLA-B58:02 | L55 |
| HLA-A02:01 | L2 | HLA-A68:02 | L20 | HLA-B41:01 | L38 | HLA-B81:01 | L56 |
| HLA-A02:03 | L3 | HLA-A74:01 | L21 | HLA-B42:01 | L39 | HLA-C01:02 | L57 |
| HLA-A02:04 | L4 | HLA-B07:02 | L22 | HLA-B44:02 | L40 | HLA-C02:02 | L58 |
| HLA-A02:07 | L5 | HLA-B08:01 | L23 | HLA-B44:03 | L41 | HLA-C03:02 | L59 |
| HLA-A03:01 | L6 | HLA-B13:02 | L24 | HLA-B45:01 | L42 | HLA-C03:03 | L60 |
| HLA-A11:01 | L7 | HLA-B14:02 | L25 | HLA-B46:01 | L43 | HLA-C03:04 | L61 |
| HLA-A23:01 | L8 | HLA-B15:01 | L26 | HLA-B48:01 | L44 | HLA-C04:01 | L62 |
| HLA-A24:02 | L9 | HLA-B15:03 | L27 | HLA-B49:01 | L45 | HLA-C05:01 | L63 |
| HLA-A25:01 | L10 | HLA-B15:09 | L28 | HLA-B50:01 | L46 | HLA-C06:02 | L64 |
| HLA-A26:01 | L11 | HLA-B18:01 | L29 | HLA-B51:01 | L47 | HLA-C07:01 | L65 |
| HLA-A29:02 | L12 | HLA-B27:05 | L30 | HLA-B52:01 | L48 | HLA-C07:02 | L66 |
| HLA-A30:01 | L13 | HLA-B35:01 | L31 | HLA-B53:01 | L49 | HLA-C08:01 | L67 |
| HLA-A30:02 | L14 | HLA-B35:03 | L32 | HLA-B54:01 | L50 | HLA-C08:02 | L68 |
| HLA-A31:01 | L15 | HLA-B37:01 | L33 | HLA-B55:01 | L51 | HLA-C12:02 | L69 |
| HLA-A32:01 | L16 | HLA-B38:01 | L34 | HLA-B57:01 | L52 | HLA-C12:03 | L70 |
| HLA-A33:03 | L17 | HLA-B39:01 | L35 | HLA-B57:03 | L53 | HLA-C14:02 | L71 |
| HLA-A36:01 | L18 | HLA-B40:01 | L36 | HLA-B58:01 | L54 | HLA-C14:03 | L72 |
| — | — | — | — | — | — | HLA-C15:02 | L73 |
| — | — | — | — | — | — | HLA-C17:01 | L74 |

TABLE 2Ai
Peptides and Alleles

| | Set 1 | Set 1 | Set 2 | Set 2 |
|---|---|---|---|---|
| | Peptide | Allele | Peptide | Allele |
| 1 | RGPEQTQGNFGDQELI | M51 | QSSYIVDSVTVKNGSI | M36;M12;M48;M36;M12; |
| | RQGTDYKHW | | HLYFDKAGQ | M48 |
| 2 | GPEQTQGNFGDQELIR | M51 | SRILGAGCFVDDIVKT | M9;M10;M6;M63;M8;M11 |
| | QGTDYKHWP | | DGTLMIERF | |
| 3 | LLNVPLHGTILTRPLLE | M28 | RILGAGCFVDDIVKTD | M9;M10;M6;M63;M8;M11 |
| | SELVIGAV | | GTLMIERFV | |
| 4 | TSSIVITSGDGTTSPISE | M28 | AALALLLLDRLNQLE | M58;M10;M53;M51;M17; |
| | HDYQIGG | | SKMSGKGQQQ | M11 |
| 5 | NILLNVPLHGTILTRPL | M28 | DAALALLLLDRLNQL | M58;M10;M53;M51;M17; |
| | LESELVIG | | ESKMSGKGQQ | M11 |
| 6 | VTSSIVITSGDGTTSPIS | M28 | SLRLIDAMMFTSDLA | M50;M36;M44;M50;M36; |
| | EHDYQIG | | TNNLVVMAYI | M44 |
| 7 | SSIVITSGDGTTSPISEH | M28 | RLIDAMMFTSDLATN | M50;M36;M44;M50;M36; |
| | DYQIGGY | | NLVVMAYITG | M44 |
| 8 | SVTSSIVITSGDGTTSPI | M28 | LRLIDAMMFTSDLAT | M50;M36;M44;M50;M36; |
| | SEHDYQI | | NNLVVMAYIT | M44 |
| 9 | ILLNVPLHGTILTRPLL | M28 | LIDAMMFTSDLATNN | M50;M36;M44;M50;M36; |
| | ESELVIGA | | LVVMAYITGG | M44 |
| 10 | VPLHGTILTRPLLESEL | M28 | YPDPSRILGAGCFVDD | M12;M57;M63;M8;M9;M20 |
| | VIGAVILR | | IVKTDGTLM | |
| 11 | SIVITSGDGTTSPISEHD | M28 | PDPSRILGAGCFVDDI | M12;M57;M63;M8;M9;M20 |
| | YQIGGYT | | VKTDGTLMI | |
| 12 | LNVPLHGTILTRPLLES | M28 | PTESIVRFPNITNLCPF | M21;M28;M66;M45;M48; |
| | ELVIGAVI | | GEVFNATR | M41 |
| 13 | IVITSGDGTTSPISEHD | M28 | VNVSLVKPSFYVYSR | M40;M71;M12;M16;M33; |
| | YQIGGYTE | | VKNLNSSRVP | M8 |
| 14 | NVPLHGTILTRPLLESE | M28 | IVNVSLVKPSFYVYSR | M40;M71;M12;M16;M33; |
| | LVIGAVIL | | VKNLNSSRV | M8 |
| 15 | FVSGNCDVVIGIVNNT | M72 | CTGYREGYLNSTNVT | M48;M14;M8;M48;M14;M8 |
| | VYDPLQPEL | | IATYCTGSIP | |
| 16 | VATEGALNTPKDHIGT | M72 | IADYNYKLPDDFTGC | M13;M12;M14;M29;M38; |
| | RNPANNAAI | | VIAWNSNNLD | M67 |
| 17 | TFVSGNCDVVIGIVNN | M72 | ATPSDFVRATATIPIQ | M14;M34;M29;M45;M33; |
| | TVYDPLQPE | | ASLPFGWLI | M9 |
| 18 | NTFVSGNCDVVIGIVN | M72 | ITREVGFVVPGLPGTI | M1;M12;M6;M1;M12;M6 |
| | NTVYDPLQP | | LRTTNGDFL | |
| 19 | IWVATEGALNTPKDHI | M72 | GDISGINASVVNIQKEI | M28;M14;M70;M80;M35; |
| | GTRNPANNA | | DRLNEVAK | M47 |
| 20 | WVATEGALNTPKDHI | M72 | DISGINASVVNIQKEID | M28;M14;M70;M80;M35; |
| | GTRNPANNAA | | RLNEVAKN | M47 |
| 21 | TEGALNTPKDHIGTRN | M72 | DGGCINANQVIVNNL | M73;M58;M50;M33;M51; |
| | PANNAAIVL | | DKSAGFPFNK | M17 |
| 22 | VSGNCDVVIGIVNNTV | M72 | YDGGCINANQVIVNN | M73;M58;M50;M33;M51; |
| | YDPLQPELD | | LDKSAGFPFN | M17 |
| 23 | ATEGALNTPKDHIGTR | M72 | NLHSSRLSFKELLVYA | M48;M57;M36;M41;M49; |
| | NPANNAAIV | | ADPAMHAAS | M20 |
| 24 | KHFFFAQDGNAAISDY | M67 | MLDMYSVMLTNDNT | M64;M7;M34;M61;M36; |
| | DYYRYNLPT | | SRYWEPEFYEA | M22 |
| 25 | FFAQDGNAAISDYDY | M67 | HMLDMYSVMLTNDN | M64;M7;M34;M61;M36; |
| | YRYNLPTMCD | | TSRYWEPEFYE | M22 |
| 26 | YQIGGYTEKWESGVK | M67 | KPSKRSFIEDLLFNKV | M73;M44;M63;M67;M11; |
| | DCVVLHSYFT | | TLADAGFIK | M47 |
| 27 | HFFFAQDGNAAISDYD | M67 | DPSKPSKRSFIEDLLF | M73;M44;M63;M67;M11; |
| | YYRYNLPTM | | NKVTLADAG | M47 |
| 28 | DYQIGGYTEKWESGV | M67 | PSKPSKRSFIEDLLFN | M73;M44;M63;M67;M11; |
| | KDCVVLHSYF | | KVTLADAGF | M47 |
| 29 | FFFAQDGNAAISDYDY | M67 | SKPSKRSFIEDLLFNK | M73;M44;M63;M67;M11; |
| | YRYNLPTMC | | VTLADAGFI | M47 |
| 30 | SHFAIGLALYYPSARIV | M5 | GMEVTPSGTWLTYTG | M19;M41;M8;M35;M11; |
| | YTACSHAA | | AIKLDDKDPN | M23 |
| 31 | HFAIGLALYYPSARIV | M5 | RTIKVFTTVDNINLHT | M51;M26;M67;M51;M26; |
| | YTACSHAAV | | QVVDMSMTY | M67 |
| 32 | SDYDYYRYNLPTMCD | M74 | GSLAATVRLQAGNAT | M55;M51;M10;M55;M51; |
| | IRQLLFVVEV | | EVPANSTVLS | M10 |
| 33 | LLNKHIDAYKTFPPTE | M41 | PINLVRDLPQGFSALE | M31;M34;M45;M36;M52; |
| | PKKDKKKKA | | PLVDLPIGI | M47 |
| 34 | LNKHIDAYKTFPPTEP | M41 | DSYYSLLMPILTLTRA | M12;M16;M58;M8;M17; |
| | KKDKKKKAD | | LTAESHVDT | M15 |
| 35 | ILLNKHIDAYKTFPPTE | M41 | CFDKFKVNSTLEQYV | M40;M29;M6;M50;M18; |
| | PKKDKKKK | | FCTVNALPET | M44 |
| 36 | RQGTDYKHWPQIAQF | M82 | FDKFKVNSTLEQYVF | M40;M29;M6;M50;M18; |
| | APSASAFFGM | | CTVNALPETT | M44 |
| 37 | IRQGTDYKHWPQIAQF | M82 | QDVNLHSSRLSFKELL | M53;M38;M48;M57;M36; |
| | APSASAFFG | | VYAADPAMH | M20 |
| 38 | QGTDYKHWPQIAQFA | M82 | PFGEVFNATRFASVY | M19;M55;M25;M53;M2; |
| | PSASAFFGMS | | AWNRKRISNC | M20 |
| 39 | LAATKMSECVLGQSK | M27 | FGEVFNATRFASVYA | M19;M55;M25;M53;M2; |
| | RVDFCGKGYH | | WNRKRISNCV | M20 |
| 40 | APATVCGPKKSTNLV | M27 | QALNTLVKQLSSNFG | M53;M6;M45;M15;M46; |
| | KNKCVNFNFN | | AISSVLNDIL | M22 |
| 41 | CGPKKSTNLVKNKCV | M27 | NRKRISNCVADYSVL | M7;M74;M67;M27;M15; |
| | NFNFNGLTGT | | YNSASFSTFK | M44 |
| 42 | VCGPKKSTNLVKNKC | M27 | KYLPIDKCSRIIPARAR | M13;M28;M14;M29;M33; |
| | VNFNFNGLTG | | VECFDKFK | M26 |
| 43 | ANLAATKMSECVLGQ | M27 | LKYLPIDKCSRIIPARA | M13;M28;M14;M29;M33; |
| | SKRVDFCGKG | | RVECFDKF | M26 |
| 44 | SFELLHAPATVCGPKK | M27 | YLPIDKCSRIIPARARV | M13;M28;M14;M29;M33; |
| | STNLVKNKC | | ECFDKFKV | M26 |
| 45 | LLHAPATVCGPKKSTN | M27 | ALKYLPIDKCSRIIPAR | M13;M28;M14;M29;M33; |
| | LVKNKCVNF | | ARVECFDK | M26 |
| 46 | FELLHAPATVCGPKKS | M27 | LPIDKCSRIIPARARVE | M13;M28;M14;M29;M33; |
| | TNLVKNKCV | | CFDKFKVN | M26 |
| 47 | NLAATKMSECVLGQS | M27 | DGVYFASTEKSNIIRG | M64;M24;M10;M6;M45; |
| | KRVDFCGKGY | | WIFGTTLDS | M22 |
| 48 | TVCGPKKSTNLVKNK | M27 | VVDSYYSLLMPILTLT | M58;M16;M8;M17;M15; |
| | CVNFNFNGLT | | RALTAESHV | M39 |
| 49 | ATVCGPKKSTNLVKN | M27 | VDSYYSLLMPILTLTR | M58;M16;M8;M17;M15; |
| | KCVNFNFNGL | | ALTAESHVD | M39 |
| 50 | HAPATVCGPKKSTNL | M27 | HTNCYDYCIPYNSVT | M12;M29;M45;M43;M62; |
| | VKNKCVNFNF | | SSIVITSGDG | M42 |
| 51 | PATVCGPKKSTNLVK | M27 | GEIKDATPSDFVRATA | M9;M34;M45;M33;M8;M11 |
| | NKCVNFNFNG | | TIPIQASLP | |
| 52 | ELLHAPATVCGPKKST | M27 | YRGTTTYKLNVGDYF | M16;M34;M43;M42;M39; |
| | NLVKNKCVN | | VLTSHTVMPL | M44 |
| 53 | LHAPATVCGPKKSTNL | M27 | IYNDKVAGFAKFLKT | M4;M73;M55;M78;M79;M8 |
| | VKNKCVNFN | | NCCRFQEKDE | |
| 54 | YYRRATRRIRGGDGK | M60 | KKPRQKRTATKAYN | M19;M70;M78;M5;M25;M2 |
| | MKDLSPRWYF | | VTQAFGRRGPE | |
| 55 | TRRIRGGDGKMKDLSP | M60 | PPGDQFKHLIPLMYK | M56;M12;M1;M60;M18; |
| | RWYFYYLGT | | GLPWNVVRIK | M39 |
| 56 | YRRATRRIRGGDGKM | M60 | FCTQLNRALTGIAVE | M28;M14;M70;M67;M27; |
| | KDLSPRWYFY | | QDKNTQEVFA | M20 |
| 57 | ATRRIRGGDGKMKDL | M60 | SFCTQLNRALTGIAVE | M28;M14;M70;M67;M27; |
| | SPRWYFYYLG | | QDKNTQEVF | M20 |
| 58 | RATRRIRGGDGKMKD | M60 | CTQLNRALTGIAVEQ | M28;M14;M70;M67;M27; |
| | LSPRWYFYYL | | DKNTQEVFAQ | M20 |
| 59 | RRATRRIRGGDGKMK | M60 | TQLNRALTGIAVEQD | M28;M14;M70;M67;M27; |
| | DLSPRWYFYY | | KNTQEVFAQV | M20 |
| 60 | YFTQSRNLQEFKPRSQ | M75 | FLKKDAPYIVGDVVQ | M7;M30;M14;M7;M30;M14 |
| | MEIDFLELA | | EGVLTAVVIP | |
| 61 | LTNDNTSRYWEPEFYE | M75 | TPSGTWLTYTGAIKL | M10;M7;M41;M8;M35;M11 |
| | AMYTPHTVL | | DDKDPNFKDQ | |
| 62 | RVIHFGAGSDKGVAPG | M13 | YRAFDIYNDKVAGFA | M4;M55;M7;M78;M79;M41 |
| | TAVLRQWLP | | KFLKTNCCRF | |
| 63 | FTQSRNLQEFKPRSQM | M75 | RAFDIYNDKVAGFAK | M4;M55;M7;M78;M79;M41 |
| | EIDFLELAM | | FLKTNCCRFQ | |
| 64 | KKKADETQALPQRQK | M13 | TTCFSVAALTNNVAF | M73;M1;M29;M6;M22;M59 |
| | KQQTVTLLPA | | QTVKPGNFNK | |
| 65 | PTEPKKDKKKKADET | M13 | CFSVAALTNNVAFQT | M73;M1;M29;M6;M22;M59 |
| | QALPQRQKKQ | | VKPGNFNKDF | |
| 66 | KADETQALPQRQKKQ | M13 | TCFSVAALTNNVAFQ | M73;M1;M29;M6;M22;M59 |
| | QTVTLLPAAD | | TVKPGNFNKD | |
| 67 | DKKKKADETQALPQR | M13 | LSYFIASFRLFARTRS | M64;M40;M16;M30;M8; |
| | QKKQQTVTLL | | MWSFNPETN | M15 |
| 68 | KKADETQALPQRQKK | M13 | WLSYFIASFRLFARTR | M64;M40;M16;M30;M8; |
| | QQTVTLLPAA | | SMWSFNPET | M15 |
| 69 | EPKKDKKKKADETQA | M13 | SYFIASFRLFARTRSM | M64;M40;M16;M30;M8; |
| | LPQRQKKQQT | | WSFNPETNI | M15 |
| 70 | TEPKKDKKKKADETQ | M13 | FIASFRLFARTRSMWS | M64;M40;M16;M30;M8; |
| | ALPQRQKKQQ | | FNPETNILL | M15 |
| 71 | KKKKADETQALPQRQ | M13 | YFIASFRLFARTRSM | M64;M40;M16;M30;M8; |
| | KKQQTVTLLP | | WSFNPETNIL | M15 |
| 72 | KKDKKKKADETQALP | M13 | ALDISASIVAGGIVAIV | M22;M12;M14;M22;M12; |
| | QRQKKQQTVT | | VTCLAYYF | M14 |
| 73 | KDKKKKADETQALPQ | M13 | LDISASIVAGGIVAIVV | M22;M12;M14;M22;M12; |
| | RQKKQQTVTL | | TCLAYYFM | M14 |
| 74 | PKKDKKKKADETQAL | M13 | FDTFNGECPNFVFPLN | M19;M45;M61;M19;M45; |
| | PQRQKKQQTV | | SIIKTIQPR | M61 |
| 75 | TQSRNLQEFKPRSQME | M75 | NVAKYTQLCQYLNTL | M78;M34;M48;M41;M8; |
| | IDFLELAMD | | TLAVPYNMRV | M49 |
| 76 | QSRNLQEFKPRSQMEI | M75 | MNVAKYTQLCQYLN | M78;M34;M48;M41;M8; |
| | DFLELAMDE | | TLTLAVPYNMR | M49 |
| 77 | PEFYEAMYTPHTVLQ | M75 | DADSKIVQLSEISMDN | M7;M36;M49;M7;M36;M49 |
| | AVGACVLCNS | | SPNLAWPLI | |
| 78 | SRNLQEFKPRSQMEID | M75 | GDKSVYYTSNPTTFH | M50;M45;M44;M50;M45; |
| | FLELAMDEF | | LDGEVITFDN | M44 |
| 79 | LHLLIGLAKRFKESPFE | M3;M20 | RGDKSVYYTSNPTTF | M50;M45;M44;M50;M45; |
| | LEDFIPMD | | HLDGEVITFD | M44 |
| 80 | SGVKDCVVLHSYFTSD | M3;M20 | IASFRLFARTRSMWSF | M64;M40;M16;M30;M8; |
| | YYQLYSTQL | | NPETNILLN | M42 |
| 81 | GLHLLIGLAKRFKESPF | M3;M20 | FDIYNDKVAGFAKFL | M4;M73;M55;M7;M78;M79 |
| | ELEDFIPM | | KTNCCRFQEK | |
| 82 | FGRRGPEQTQGNFGD | M51;M63 | TTIKPVTYKLDGVVC | M36;M6;M59;M36;M6;M59 |
| | QELIRQGTDY | | TEIDPKLDNY | |
| 83 | RRGPEQTQGNFGDQE | M51;M63 | SVAALTNNVAFQTVK | M73;M1;M25;M29;M6;M59 |
| | LIRQGTDYKH | | PGNFNKDFYD | |
| 84 | GRRGPEQTQGNFGDQ | M51;M63 | KLSYGIATVREVLSDR | M7;M5;M6;M49;M11;M59 |
| | ELIRQGTDYK | | ELHLSWEVG | |
| 85 | TVSCLPFTINCQEPKLG | M50;M51 | SGFKLKDCVMYASA | M33;M12;M14;M33;M12; |
| | SLVVRCSF | | VVLLILMTART | M14 |
| 86 | NYTVSCLPFTINCQEP | M50;M51 | GFKLKDCVMYASAV | M33;M12;M14;M33;M12; |
| | KLGSLVVRC | | VLLILMTARTV | M14 |
| 87 | GNYTVSCLPFTINCQE | M50;M51 | SLSGFKLKDCVMYAS | M33;M12;M14;M33;M12; |
| | PKLGSLVVR | | AVVLLILMTA | M14 |
| 88 | VSCLPFTINCQEPKLGS | M50;M51 | LSGFKLKDCVMYASA | M33;M12;M14;M33;M12; |
| | LVVRCSFY | | VVLLILMTAR | M14 |
| 89 | YTVSCLPFTINCQEPKL | M50;M51 | DLLIRKSNHNFLVQA | M12;M14;M70;M12;M14; |
| | GSLVVRCS | | GNVQLRVIGH | M70 |
| 90 | GTSPARMAGNGGDAA | M51;M13 | LLIRKSNHNFLVQAG | M12;M14;M70;M12;M14; |
| | LALLLLDRLN | | NVQLRVIGHS | M70 |
| 91 | TSPARMAGNGGDAAL | M51;M13 | LIRKSNHNFLVQAGN | M12;M14;M70;M12;M14; |
| | ALLLLDRLNQ | | VQLRVIGHSM | M70 |
| 92 | PLSAPTLVPQEHYVRIT | M51;M69 | TQQLIRAAEIRASANL | M64;M40;M12;M48;M22; |
| | GLYPTLNI | | AATKMSECV | M23 |
| 93 | SPARMAGNGGDAALA | M51;M13 | QALPQRQKKQQTVTL | M54;M14;M60;M29;M26; |
| | LLLLDRLNQL | | LPAADLDDFS | M39 |
| 94 | FYYLGTGPEAGLPYGA | M12;M6 | FLNRFTTTLNDFNLV | M14;M63;M66;M14;M63; |
| | NKDGIIWVA | | AMKYNYEPLT | M66 |
| 95 | YFYYLGTGPEAGLPYG | M12;M6 | WFLNRFTTTLNDFNL | M14;M63;M66;M14;M63; |
| | ANKDGIIWV | | VAMKYNYEPL | M66 |
| 96 | ELKHFFFAQDGNAAIS | M36;M67 | DRWFLNRFTTTLNDF | M14;M63;M66;M14;M63; |
| | DYDYYRYNL | | NLVAMKYNYE | M66 |
| 97 | LKHFFFAQDGNAAISD | M36;M67 | NGDRWFLNRFTTTLN | M14;M63;M66;M14;M63; |
| | YDYYRYNLP | | DFNLVAMKYN | M66 |
| 98 | SVMLTNDNTSRYWEP | M36;M22 | GDRWFLNRFTTTLND | M14;M63;M66;M14;M63; |
| | EFYEAMYTPH | | FNLVAMKYNY | M66 |
| 99 | KEGQINDMILSLLSKG | M55;M19 | VINGDRWFLNRFTTT | M14;M63;M66;M14;M63; |
| | RLIIRENNR | | LNDFNLVAMK | M66 |
| 100 | EGQINDMILSLLSKGR | M55;M19 | RWFLNRFTTTLNDFN | M14;M63;M66;M14;M63; |
| | LIIRENNRV | | LVAMKYNYEP | M66 |
| 101 | LKEGQINDMILSLLSK | M55;M19 | INGDRWFLNRFTTTL | M14;M63;M66;M14;M63; |
| | GRLIIRENN | | NDFNLVAMKY | M66 |
| 102 | KNHTSPDVDLGDISGI | M80;M45 | LIRAAEIRASANLAAT | M4;M64;M40;M48;M22; |
| | NASVVNIQK | | KMSECVLGQ | M23 |
| 103 | IVQMLSDTLKNLSDRV | M36;M61 | QLIRAAEIRASANLAA | M4;M64;M40;M48;M22; |
| | VFVLWAHGF | | TKMSECVLG | M23 |
| 104 | KIVQMLSDTLKNLSDR | M36;M61 | KCSRIIPARARVECFD | M28;M10;M50;M33;M9; |
| | VVFVLWAHG | | KFKVNSTLE | M44 |
| 105 | PHEFCSQHTMLVKQG | M29;M14 | CSRIIPARARVECFDK | M28;M10;M50;M33;M9; |
| | DDYVYLPYPD | | FKVNSTLEQ | M44 |
| 106 | HEFCSQHTMLVKQGD | M29;M14 | TVLPPLLTDEMIAQYT | M7;M38;M48;M41;M49; |
| | DYVYLPYPDP | | SALLAGTIT | M26 |
| 107 | TGPEAGLPYGANKDGI | M57;M14 | VLPPLLTDEMIAQYTS | M7;M38;M48;M41;M49; |
| | IWVATEGAL | | ALLAGTITS | M26 |
| 108 | GYTEKWESGVKDCVV | M67;M14 | LKITEEVGHTDLMAA | M63;M14;M70;M63;M14; |
| | LHSYFTSDYY | | YVDNSSLTIK | M70 |
| 109 | SYSLFDMSKFPLKLRG | M67;M14 | NFRVQPTESIVRFPNIT | M21;M28;M48;M41;M26; |
| | TAVMSLKEG | | NLCPFGEV | M22 |
| 110 | IGGYTEKWESGVKDC | M67;M14 | FRVQPTESIVRFPNITN | M21;M28;M48;M41;M26; |
| | VVLHSYFTSD | | LCPFGEVF | M22 |
| 111 | YSLFDMSKFPLKLRGT | M67;M14 | KGYGCSCDQLREPML | M33;M12;M45;M33;M12; |
| | AVMSLKEGQ | | QSADAQSFLN | M45 |
| 112 | QIGGYTEKWESGVKD | M67;M14 | RELMRELNGGAYTRY | M63;M14;M41;M63;M14; |
| | CVVLHSYFTS | | VDNNFCGPDG | M41 |
| 113 | GGYTEKWESGVKDCV | M67;M14 | MRELNGGAYTRYVD | M63;M14;M41;M63;M14; |
| | VLHSYFTSDY | | NNFCGPDGYPL | M41 |
| 114 | DGIIWVATEGALNTPK | M72;M6 | LMRELNGGAYTRYV | M63;M14;M41;M63;M14; |
| | DHIGTRNPA | | DNNFCGPDGYP | M41 |
| 115 | IIWVATEGALNTPKDH | M72;M6 | ELMRELNGGAYTRY | M63;M14;M41;M63;M14; |
| | IGTRNPANN | | VDNNFCGPDGY | M41 |
| 116 | GIIWVATEGALNTPKD | M72;M6 | RELNGGAYTRYVDN | M63;M14;M41;M63;M14; |
| | HIGTRNPAN | | NFCGPDGYPLE | M41 |
| 117 | HSQLGGLHLLIGLAKR | M19;M61 | LSPRWYFYYLGTGPE | M32;M12;M31;M6;M68; |
| | FKESPFELE | | AGLPYGANKD | M52 |
| 118 | DFSHSQLGGLHLLIGL | M19;M61 | TSLSGFKLKDCVMYA | M75;M12;M14;M75;M12; |
| | AKRFKESPF | | SAVVLLILMT | M14 |
| 119 | FSHSQLGGLHLLIGLA | M19;M61 | TNNTFTLKGGAPTKV | M63;M11;M12;M63;M11; |
| | KRFKESPFE | | TFGDDTVIEV | M12 |
| 120 | GDFSHSQLGGLHLLIG | M19;M61 | NTFTLKGGAPTKVTF | M63;M11;M12;M63;M11; |
| | LAKRFKESP | | GDDTVIEVQG | M12 |
| 121 | SHSQLGGLHLLIGLAK | M19;M61 | NNTFTLKGGAPTKVT | M63;M11;M12;M63;M11; |
| | RFKESPFEL | | FGDDTVIEVQ | M12 |
| 122 | YGDFSHSQLGGLHLLI | M19;M61 | VTNNTFTLKGGAPTK | M63;M11;M12;M63;M11; |
| | GLAKRFKES | | VTFGDDTVIE | M12 |
| 123 | TDNTFVSGNCDVVIGI | M72;M12 | TDLMAAYVDNSSLTI | M63;M9;M44;M63;M9;M44 |
| | VNNTVYDPL | | KKPNELSRVL | |
| 124 | TTDNTFVSGNCDVVIG | M72;M12 | RQKKQQTVTLLPAAD | M54;M14;M60;M46;M26; |
| | IVNNTVYDP | | LDDFSKQLQQ | M39 |
| 125 | ITTDNTFVSGNCDVVI | M72;M12 | MPSYCTGYREGYLNS | M49;M45;M8;M49;M45;M8 |
| | GIVNNTVYD | | TNVTIATYCT | |
| 126 | DNTFVSGNCDVVIGIV | M72;M12 | TTEMLAKALRKVPTD | M48;M14;M49;M48;M14; |
| | NNTVYDPLQ | | NYITTYPGQG | M49 |
| 127 | DAVVYRGTTTYKLNV | M42;M44 | TEMLAKALRKVPTDN | M48;M14;M49;M48;M14; |
| | GDYFVLTSHT | | YITTYPGQGL | M49 |
| 128 | AVVYRGTTTYKLNVG | M42;M44 | TETDLTKGPHEFCSQ | M30;M6;M18;M43;M42; |
| | DYFVLTSHTV | | HTMLVKQGDD | M39 |
| 129 | DLKFPRGQGVPINTNS | M73;M44 | YHKNNKSWMESEFR | M70;M5;M2;M48;M41;M82 |
| | SPDDQIGYY | | VYSSANNCTFE | |
| 130 | KFPRGQGVPINTNSSP | M73;M44 | ELTGHMLDMYSVML | M64;M31;M34;M61;M36; |
| | DDQIGYYRR | | TNDNTSRYWEP | M22 |
| 131 | EDLKFPRGQGVPINTN | M73;M44 | MESEFRVYSSANNCT | M64;M5;M2;M48;M41;M82 |
| | SSPDDQIGY | | FEYVSQPFLM | |
| 132 | LKFPRGQGVPINTNSSP | M73;M44 | KNNKSWMESEFRVYS | M64;M5;M2;M48;M41;M82 |
| | DDQIGYYR | | SANNCTFEYV | |
| 133 | FPRGQGVPINTNSSPD | M73;M44 | NKSWMESEFRVYSSA | M64;M5;M2;M48;M41;M82 |
| | DQIGYYRRA | | NNCTFEYVSQ | |
| 134 | PRGQGVPINTNSSPDD | M73;M44 | KSWMESEFRVYSSAN | M64;M5;M2;M48;M41;M82 |
| | QIGYYRRAT | | NCTFEYVSQP | |
| 135 | GDAVVYRGTTTYKLN | M9;M44 | NNKSWMESEFRVYSS | M64;M5;M2;M48;M41;M82 |
| | VGDYFVLTSH | | ANNCTFEYVS | |
| 136 | RTCCLCDRRATCFSTA | M4;M72 | SWMESEFRVYSSANN | M64;M5;M2;M48;M41;M82 |
| | SDTYACWHH | | CTFEYVSQPF | |
| 137 | CDRRATCFSTASDTYA | M4;M72 | WMESEFRVYSSANNC | M64;M5;M2;M48;M41;M82 |
| | CWHHSIGFD | | TFEYVSQPFL | |
| 138 | LCDRRATCFSTASDTY | M4;M72 | GLPWNVVRIKIVQML | M61;M57;M36;M46;M9; |
| | ACWHHSIGF | | SDTLKNLSDR | M20 |
| 139 | CLCDRRATCFSTASDT | M4;M72 | KGLPWNVVRIKIVQM | M61;M57;M36;M46;M9; |
| | YACWHHSIG | | LSDTLKNLSD | M20 |
| 140 | CCLCDRRATCFSTASD | M4;M72 | YKGLPWNVVRIKIVQ | M61;M57;M36;M46;M9; |
| | TYACWHHSI | | MLSDTLKNLS | M20 |
| 141 | TCCLCDRRATCFSTAS | M4;M72 | DDYIATNGPLKVGGS | M80;M29;M14;M80;M29; |
| | DTYACWHHS | | CVLSGHNLAK | M14 |
| 142 | QVILLNKHIDAYKTFP | M41;M14 | VNARLRAKHYVYIGD | M40;M31;M1;M30;M37; |
| | PTEPKKDKK | | PAQLPAPRTL | M57 |
| 143 | VILLNKHIDAYKTFPPT | M41;M14 | VVNARLRAKHYVYIG | M40;M31;M1;M30;M37; |
| | EPKKDKKK | | DPAQLPAPRT | M57 |
| 144 | LPKGFYAEGSRGGSQA | M5;M45 | LFTRFFYVLGLAAIM | M5;M48;M45;M5;M48;M45 |
| | SSRSSSRSR | | QLFFSYFAVH | |
| 145 | TLPKGFYAEGSRGGSQ | M5;M45 | FTRFFYVLGLAAIMQ | M5;M48;M45;M5;M48;M45 |
| | ASSRSSSRS | | LFFSYFAVHF | |
| 146 | TKVDGVDVELFENKT | M29;M48 | ASYQTQTNSPRRARS | M31;M5;M2;M6;M50;M18 |
| | TLPVNVAFEL | | VASQSIIAYT | |
| 147 | YTKVDGVDVELFENK | M29;M48 | TVGQQDGSEDNQTTT | M14;M28;M59;M14;M28; |
| | TTLPVNVAFE | | IQTIVEVQPQ | M59 |
| 148 | QILPDPSKPSKRSFIED | M63;M67 | VGQQDGSEDNQTTTI | M14;M28;M59;M14;M28; |
| | LLFNKVTL | | QTIVEVQPQL | M59 |
| 149 | ILPDPSKPSKRSFIEDLL | M63;M67 | SEDNQTTTIQTIVEVQ | M14;M28;M59;M14;M28; |
| | FNKVTLA | | PQLEMELTP | M59 |
| 150 | QSKRVDFCGKGYHLM | M1;M52 | DGSEDNQTTTIQTIVE | M14;M28;M59;M14;M28; |
| | SFPQSAPHGV | | VQPQLEMEL | M59 |
| 151 | LGQSKRVDFCGKGYH | M1;M52 | QQDGSEDNQTTTIQTI | M14;M28;M59;M14;M28; |
| | LMSFPQSAPH | | VEVQPQLEM | M59 |
| 152 | GQSKRVDFCGKGYHL | M1;M52 | QDGSEDNQTTTIQTIV | M14;M28;M59;M14;M28; |
| | MSFPQSAPHG | | EVQPQLEME | M59 |
| 153 | LIIKNLSKSLTENKYSQ | M11;M28 | GSEDNQTTTIQTIVEV | M14;M28;M59;M14;M28; |
| | LDEEQPME | | QPQLEMELT | M59 |
| 154 | IIKNLSKSLTENKYSQL | M11;M28 | GQQDGSEDNQTTTIQ | M14;M28;M59;M14;M28; |
| | DEEQPMEI | | TIVEVQPQLE | M59 |
| 155 | GVDVELFENKTTLPVN | M29;M28 | VSTSGRWVLNNDYY | M33;M39;M44;M33;M39; |
| | VAFELWAKR | | RSLPGVFCGVD | M44 |
| 156 | VDVELFENKTTLPVNV | M29;M28 | NLQHRLYECLYRNRD | M6;M2;M59;M35;M52;M47 |
| | AFELWAKRN | | VDTDFVNEFY | |
| 157 | DVELFENKTTLPVNVA | M29;M28 | KATNNAMQVESDDYI | M29;M11;M44;M29;M11; |
| | FELWAKRNI | | ATNGPLKVGG | M44 |
| 158 | ELFENKTTLPVNVAFE | M29;M28 | CNVDRYPANSIVCRF | M5;M2;M48;M50;M18;M9 |
| | LWAKRNIKP | | DTRVLSNLNL | |
| 159 | LFENKTTLPVNVAFEL | M29;M28 | NCNVDRYPANSIVCR | M5;M2;M48;M50;M18;M9 |
| | WAKRNIKPV | | FDTRVLSNLN | |
| 160 | VELFENKTTLPVNVAF | M29;M28 | NVDRYPANSIVCRFD | M5;M2;M48;M50;M18;M9 |
| | ELWAKRNIK | | TRVLSNLNLP | |
| 161 | VAAGLEAPFLYLYAL | M54;M12 | ATLPKGIMMNVAKYT | M73;M58;M78;M34;M11; |
| | VYFLQSINFV | | QLCQYLNTLT | M44 |
| 162 | LVAAGLEAPFLYLYAL | M54;M12 | LGLAAIMQLFFSYFA | M48;M9;M45;M48;M9;M45 |
| | VYFLQSINF | | VHFISNSWLM | |
| 163 | IVLQLPQGTTLPKGFY | M75;M45 | GISQYSLRLIDAMMFT | M57;M36;M54;M57;M36; |
| | AEGSRGGSQ | | SDLATNNLV | M54 |
| 164 | AIVLQLPQGTTLPKGF | M75;M45 | ISQYSLRLIDAMMFTS | M57;M36;M54;M57;M36; |
| | YAEGSRGGS | | DLATNNLVV | M54 |
| 165 | VLQLPQGTTLPKGFYA | M75;M45 | SQYSLRLIDAMMFTS | M57;M36;M54;M57;M36; |
| | EGSRGGSQA | | DLATNNLVVM | M54 |
| 166 | LQLPQGTTLPKGFYAE | M75;M45 | SNNLDSKVGGNYNYL | M71;M16;M1;M37;M18; |
| | GSRGGSQAS | | YRLFRKSNLK | M15 |
| 167 | AATKMSECVLGQSKR | M1;M27 | NSNNLDSKVGGNYN | M71;M16;M1;M37;M18; |
| | VDFCGKGYHL | | YLYRLFRKSNL | M15 |
| 168 | GGNYNYLYRLFRKSN | M1;M37 | WNSNNLDSKVGGNY | M71;M16;M1;M37;M18; |
| | LKPFERDIST | | NYLYRLFRKSN | M15 |
| 169 | RVYSTGSNVFQTRAG | M75;M8 | NNLDSKVGGNYNYL | M71;M16;M1;M37;M18; |
| | CLIGAEHVNN | | YRLFRKSNLKP | M15 |
| 170 | WRVYSTGSNVFQTRA | M75;M8 | CPLIAAVITREVGFVV | M32;M63;M12;M32;M63; |
| | GCLIGAEHVN | | PGLPGTILR | M12 |
| 171 | VYSTGSNVFQTRAGCL | M75;M8 | DLFLPFFSNVTWFHAI | M29;M45;M49;M15;M46; |
| | IGAEHVNNS | | HVSGTNGTK | M26 |
| 172 | YSTGSNVFQTRAGCLI | M75;M8 | QDLFLPFFSNVTWFH | M29;M45;M49;M15;M46; |
| | GAEHVNNSY | | AIHVSGTNGT | M26 |
| 173 | STGSNVFQTRAGCLIG | M75;M8 | TQDLFLPFFSNVTWF | M29;M45;M49;M15;M46; |
| | AEHVNNSYE | | HAIHVSGTNG | M26 |
| 174 | TWRVYSTGSNVFQTR | M75;M8 | YDKLQFTSLEIPRRNV | M28;M6;M38;M27;M59; |
| | AGCLIGAEHV | | ATLQAENVT | M20 |
| 175 | SANLAATKMSECVLG | M4;M27 | DLYDKLQFTSLEIPRR | M28;M6;M38;M27;M59; |
| | QSKRVDFCGK | | NVATLQAEN | M22 |
| 176 | ASANLAATKMSECVL | M4;M27 | LYDKLQFTSLEIPRRN | M28;M6;M38;M27;M59; |
| | GQSKRVDFCG | | VATLQAENV | M20 |
| 177 | RASANLAATKMSECV | M4;M27 | LSTDGNKIADKYVRN | M54;M60;M57;M27;M44; |
| | LGQSKRVDFC | | LQHRLYECLY | M20 |
| 178 | HPTQAPTHLSVDTKFK | M7;M35 | ALLSTDGNKIADKYV | M54;M60;M57;M27;M44; |
| | TEGLCVDIP | | RNLQHRLYEC | M20 |
| 179 | LHPTQAPTHLSVDTKF | M7;M35 | LLSTDGNKIADKYVR | M54;M60;M57;M27;M44; |
| | KTEGLCVDI | | NLQHRLYECL | M20 |
| 180 | TQAPTHLSVDTKFKTE | M7;M35 | STDGNKIADKYVRNL | M54;M60;M57;M27;M44; |
| | GLCVDIPGI | | QHRLYECLYR | M20 |
| 181 | PTQAPTHLSVDTKFKT | M7;M35 | DEISMATNYDLSVVN | M19;M25;M50;M18;M33; |
| | EGLCVDIPG | | ARLRAKHYVY | M26 |
| 182 | TGLHPTQAPTHLSVDT | M7;M35 | FDEISMATNYDLSVV | M19;M25;M50;M18;M33; |
| | KFKTEGLCV | | NARLRAKHYV | M26 |
| 183 | GLHPTQAPTHLSVDTK | M7;M35 | MYSFVSEETGTLIVNS | M56;M73;M50;M18;M11; |
| | FKTEGLCVD | | VLLFLAFVV | M44 |
| 184 | SIVCRFDTRVLSNLNLP | M5;M2 | GVSICSTMTNRQFHQ | M64;M1;M60;M30;M37; |
| | GCDGGSLY | | KLLKSIAATR | M22 |
| 185 | TLMIERFVSLAIDAYPL | M5;M2 | VSICSTMTNRQFHQK | M64;M1;M60;M30;M37; |
| | TKHPNQEY | | LLKSIAATRG | M22 |
| 186 | AAEASKKPRQKRTAT | M5;M2 | VSFCYMHHMELPTGV | M43;M6;M28;M43;M6;M28 |
| | KAYNVTQAFG | | HAGTDLEGNF | |
| 187 | ANSIVCRFDTRVLSNL | M5;M2 | DGYPLECIKDLLARA | M64;M12;M28;M64;M12; |
| | NLPGCDGGS | | GKASCTLSEQ | M28 |
| 188 | NSIVCRFDTRVLSNLN | M5;M2 | WDYPKCDRAMPNML | M21;M12;M41;M46;M39; |
| | LPGCDGGSL | | RIMASLVLARK | M23 |
| 189 | LMIERFVSLAIDAYPLT | M5;M2 | MFDAYVNTFSSTFNV | M28;M8;M70;M28;M8;M70 |
| | KHPNQEYA | | PMEKLKTLVA | |
| 190 | QHRLYECLYRNRDVD | M6;M59 | FDAYVNTFSSTFNVP | M28;M8;M70;M28;M8;M70 |
| | TDFVNEFYAY | | MEKLKTLVAT | |
| 191 | LYECLYRNRDVDTDF | M6;M59 | VKMFDAYVNTFSSTF | M28;M8;M70;M28;M8;M70 |
| | VNEFYAYLRK | | NVPMEKLKTL | |
| 192 | VCRLMKTIGPDMFLGT | M6;M37 | KMFDAYVNTFSSTFN | M28;M8;M70;M28;M8;M70 |
| | CRRCPAEIV | | VPMEKLKTLV | |
| 193 | RLYECLYRNRDVDTD | M6;M59 | DAYVNTFSSTFNVPM | M28;M8;M70;M28;M8;M70 |
| | FVNEFYAYLR | | EKLKTLVATA | |
| 194 | HRLYECLYRNRDVDT | M6;M59 | LPTGVHAGTDLEGNF | M6;M28;M37;M6;M28;M37 |
| | DFVNEFYAYL | | YGPFVDRQTA | |
| 195 | PQIITTDNTFVSGNCDV | M12;M23 | MELPTGVHAGTDLEG | M6;M28;M37;M6;M28;M37 |
| | VIGIVNNT | | NFYGPFVDRQ | |
| 196 | MPNLYKMQRMLLEKC | M12;M23 | HMELPTGVHAGTDLE | M6;M28;M37;M6;M28;M37 |
| | DLQNYGDSAT | | GNFYGPFVDR | |
| 197 | FIKGLNNLNRGMVLGS | M12;M12 | ELPTGVHAGTDLEGN | M6;M28;M37;M6;M28;M37 |
| | LAATVRLQA | | FYGPFVDRQT | |
| 198 | SDLATNNLVVMAYIT | M12;M12 | IGAGICASYQTQTNSP | M19;M31;M70;M25;M50; |
| | GGVVQLTSQW | | RRARSVASQ | M18 |
| 199 | YFIKGLNNLNRGMVL | M12;M12 | PIGAGICASYQTQTNS | M19;M31;M70;M25;M50; |
| | GSLAATVRLQ | | PRRARSVAS | M18 |
| 200 | IDKCSRIIPARARVECF | M33;M28 | CTNYMPYFFTLLLQL | M25;M29;M19;M25;M29; |
| | DKFKVNST | | CTFTRSTNSR | M19 |
| 201 | RFYRLANECAQVLSE | M79;M28 | TNYMPYFFTLLLQLC | M25;M29;M19;M25;M29; |
| | MVMCGGSLYV | | TFTRSTNSRI | M19 |
| 202 | FYRLANECAQVLSEM | M79;M28 | SMATNYDLSVVNARL | M19;M25;M37;M50;M18; |
| | VMCGGSLYVK | | RAKHYVYIGD | M26 |
| 203 | NSYECDIPIGAGICASY | M28;M70 | MATNYDLSVVNARL | M19;M25;M37;M50;M18; |
| | QTQTNSPR | | RAKHYVYIGDP | M26 |
| 204 | QIHTIDGSSGVVNPVM | M38;M28 | NARDGCVPLNIIPLTT | M22;M19;M24;M22;M19; |
| | EPIYDEPTT | | AAKLMVVIP | M24 |
| 205 | HVQIHTIDGSSGVVNP | M38;M28 | NNARDGCVPLNIIPLT | M22;M19;M24;M22;M19; |
| | VMEPIYDEP | | TAAKLMVVI | M24 |
| 206 | EHVQIHTIDGSSGVVN | M38;M28 | GPLKVGGSCVLSGHN | M80;M14;M37;M80;M14; |
| | PVMEPIYDE | | LAKHCLHVVG | M37 |
| 207 | HTIDGSSGVVNPVMEP | M38;M28 | VAIVVTCLAYYFMRF | M71;M6;M16;M71;M6;M16 |
| | IYDEPTTTT | | RRAFGEYSHV | |
| 208 | EEHVQIHTIDGSSGVV | M38;M28 | GTGTIYTELEPPCRFV | M6;M2;M59;M6;M2;M59 |
| | NPVMEPIYD | | TDTPKGPKV | |
| 209 | IHTIDGSSGVVNPVME | M38;M28 | DGTGTIYTELEPPCRF | M6;M2;M59;M6;M2;M59 |
| | PIYDEPTTT | | VTDTPKGPK | |
| 210 | VQIHTIDGSSGVVNPV | M38;M28 | TGTIYTELEPPCRFVT | M6;M2;M59;M6;M2;M59 |
| | MEPIYDEPT | | DTPKGPKVK | |
| 211 | PDDQIGYYRRATRRIR | M71;M16 | EFLKRGDKSVYYTSN | M50;M22;M44;M50;M22; |
| | GGDGKMKDL | | PTTFHLDGEV | M44 |
| 212 | SPDDQIGYYRRATRRI | M71;M16 | FLKRGDKSVYYTSNP | M50;M22;M44;M50;M22; |
| | RGGDGKMKD | | TTFHLDGEVI | M44 |
| 213 | PINTNSSPDDQIGYYRR | M55;M71 | ETKFLTENLLLYIDIN | M50;M36;M70;M50;M36; |
| | ATRRIRGG | | GNLHPDSAT | M70 |
| 214 | INTNSSPDDQIGYYRR | M55;M71 | EETKFLTENLLLYIDI | M50;M36;M70;M50;M36; |
| | ATRRIRGGD | | NGNLHPDSA | M70 |
| 215 | TNILLNVPLHGTILTRP | M73;M28 | EWSMATYYLFDESGE | M52;M36;M23;M52;M36; |
| | LLESELVI | | FKLASHMYCS | M23 |
| 216 | PETNILLNVPLHGTILT | M73;M28 | MATYYLFDESGEFKL | M52;M36;M23;M52;M36; |
| | RPLLESEL | | ASHMYCSFYP | M23 |
| 217 | ETNILLNVPLHGTILTR | M73;M28 | WSMATYYLFDESGEF | M52;M36;M23;M52;M36; |
| | PLLESELV | | KLASHMYCSF | M23 |
| 218 | PLEFGATSAALQPEEE | M28;M28 | SMATYYLFDESGEFK | M52;M36;M23;M52;M36; |
| | QEEDWLDDD | | LASHMYCSFY | M23 |
| 219 | LEFGATSAALQPEEEQ | M28;M28 | LKKRWQLALSKGVH | M50;M33;M8;M42;M11; |
| | EEDWLDDDS | | FVCNLLLLFVT | M23 |
| 220 | EFGATSAALQPEEEQE | M28;M28 | EQYVFCTVNALPETT | M40;M29;M38;M6;M18; |
| | EDWLDDDSQ | | ADIVVFDEIS | M49 |
| 221 | IQTIVEVQPQLEMELTP | M28;M28 | LEQYVFCTVNALPET | M40;M29;M38;M6;M18; |
| | VVQTIEVN | | TADIVVFDEI | M49 |
| 222 | FGLYKNTCVGSDNVT | M28;M59 | ARIVYTACSHAAVDA | M28;M60;M29;M38;M67; |
| | DFNAIATCDW | | LCEKALKYLP | M49 |
| 223 | TIQTIVEVQPQLEMELT | M28;M28 | TFTRSTNSRIKASMPT | M33;M11;M14;M33;M11; |
| | PVVQTIEV | | TIAKNTVKS | M14 |
| 224 | TTIQTIVEVQPQLEMEL | M28;M28 | FTRSTNSRIKASMPTTI | M33;M11;M14;M33;M11; |
| | TPVVQTIE | | AKNTVKSV | M14 |
| 225 | FGATSAALQPEEEQEE | M28;M28 | IGIVNNTVYDPLQPEL | M70;M34;M63;M72;M59; |
| | DWLDDDSQQ | | DSFKEELDK | M47 |
| 226 | FVNEFYAYLRKHFSM | M57;M71 | VNNTVYDPLQPELDS | M70;M34;M63;M72;M59; |
| | MILSDDAVVC | | FKEELDKYFK | M47 |
| 227 | VNEFYAYLRKHFSMM | M57;M71 | IVNNTVYDPLQPELDS | M70;M34;M63;M72;M59; |
| | ILSDDAVVCF | | FKEELDKYF | 4M7 |
| 228 | ATRGATVVIGTSKFYG | M72;M17 | GIVNNTVYDPLQPEL | M70;M34;M63;M72;M59; |
| | GWHNMLKTV | | DSFKEELDKY | M47 |
| 229 | AATRGATVVIGTSKFY | M72;M17 | GYPNMFITREEAIRHV | M64;M32;M14;M57;M69; |
| | GGWHNMLKT | | RAWIGFDVE | M20 |
| 230 | TRGATVVIGTSKFYGG | M72;M17 | CLWSTKPVETSNSFD | M39;M6;M41;M39;M6;M41 |
| | WHNMLKTVY | | VLKSEDAQGM | |
| 231 | KVVKVTIDYTEISFML | M71;M67 | QAMTQMYKQARSED | M25;M19;M74;M25;M19; |
| | WCKDGHVET | | KRAKVTSAMQT | M74 |
| 232 | NLLLDKRTTCFSVAAL | M29;M22 | AMTQMYKQARSEDK | M25;M19;M74;M25;M19; |
| | TNNVAFQTV | | RAKVTSAMQTM | M74 |
| 233 | SGNLLLDKRTTCFSVA | M29;M22 | DQAMTQMYKQARSE | M25;M19;M74;M25;M19; |
| | ALTNNVAFQ | | DKRAKVTSAMQ | M74 |
| 234 | GNLLLDKRTTCFSVAA | M29;M22 | RHVICTSEDMLNPNY | M29;M72;M28;M29;M72; |
| | LTNNVAFQT | | EDLLIRKSNH | M28 |
| 235 | ATVVIGTSKFYGGWH | M18;M72 | HVICTSEDMLNPNYE | M29;M72;M28;M29;M72; |
| | NMLKTVYSDV | | DLLIRKSNHN | M28 |
| 236 | QINDMILSLLSKGRLII | M55;M9 | RQVVNVVTTKIALKG | M57;M29;M12;M57;M29; |
| | RENNRVVI | | GKIVNNWLKQ | M12 |
| 237 | AQDGNAAISDYDYYR | M67;M74 | NVVTTKIALKGGKIV | M57;M11;M12;M57;M11; |
| | YNLPTMCDIR | | NNWLKQLIKV | M12 |
| 238 | QDGNAAISDYDYYRY | M67;M74 | VNVVTTKIALKGGKI | M57;M11;M12;M57;M11; |
| | NLPTMCDIRQ | | VNNWLKQLIK | M12 |
| 239 | FAQDGNAAISDYDYY | M67;M74 | TDPYEDFQENWNTKH | M44;M61;M74;M44;M61; |
| | RYNLPTMCDI | | SSGVTRELMR | M74 |
| 240 | VLSNLNLPGCDGGSLY | M11;M73 | ERSEAGVCVSTSGRW | M38;M24;M44;M38;M24; |
| | VNKHAFHTP | | VLNNDYYRSL | M44 |
| 241 | RVLSNLNLPGCDGGSL | M11;M73 | RSEAGVCVSTSGRWV | M38;M24;M44;M38;M24; |
| | YVNKHAFHT | | LNNDYYRSLP | M44 |
| 242 | TRVLSNLNLPGCDGGS | M11;M73 | CERSEAGVCVSTSGR | M38;M24;M44;M38;M24; |
| | LYVNKHAFH | | WVLNNDYYRS | M44 |
| 243 | EFYAYLRKHFSMMILS | M57;M13 | SEAGVCVSTSGRWVL | M38;M24;M44;M38;M24; |
| | DDAVVCFNS | | NNDYYRSLPG | M44 |
| 244 | RNRATRVECTTIVNGV | M29;M29 | TCERSEAGVCVSTSG | M38;M24;M44;M38;M24; |
| | RRSFYVYAN | | RWVLNNDYYR | M44 |
| 245 | YKRNRATRVECTTIVN | M29;M29 | LTYNKVENMTPRDLG | M14;M67;M74;M14;M67; |
| | GVRRSFYVY | | ACIDCSARHI | M74 |
| 246 | ADETQALPQRQKKQQ | M13;M29 | MLTYNKVENMTPRD | M14;M67;M74;M14;M67; |
| | TVTLLPAADL | | LGACIDCSARH | M74 |
| 247 | KRNRATRVECTTIVNG | M29;M29 | PLKLRGTAVMSLKEG | M54;M60;M6;M67;M49; |
| | VRRSFYVYA | | QINDMILSLL | M59 |
| 248 | VNVAFELWAKRNIKP | M66;M74 | YPNASFDNFKFVCDNI | M64;M25;M19;M64;M25; |
| | VPEVKILNNL | | KFADDLNQL | M19 |
| 249 | CEEEEFEPSTQYEYGT | M67;M67 | DLGDELGTDPYEDFQ | M44;M61;M70;M44;M61; |
| | EDDYQGKPL | | ENWNTKHSSG | M70 |
| 250 | NINLHTQVVDMSMTY | M67;M67 | GDELGTDPYEDFQEN | M44;M61;M70;M44;M61; |
| | GQQFGPTYLD | | WNTKHSSGVT | M70 |
| 251 | FGWLIVGVALLAVFQS | M26;M61 | FDLGDELGTDPYEDF | M44;M61;M70;M44;M61; |
| | ASKIITLKK | | QENWNTKHSS | M70 |
| 252 | GWLIVGVALLAVFQS | M26;M61 | SFDLGDELGTDPYED | M44;M61;M70;M44;M61; |
| | ASKIITLKKR | | FQENWNTKHS | M70 |
| 253 | LNTLTLAVPYNMRVIH | M5;M70 | LGDELGTDPYEDFQE | M44;M61;M70;M44;M61; |
| | FGAGSDKGV | | NWNTKHSSGV | M70 |
| 254 | NTLTLAVPYNMRVIHF | M5;M70 | SVTSNYSGVVTTVMF | M29;M38;M14;M29;M38; |
| | GAGSDKGVA | | LARGIVFMCV | M14 |
| 255 | TLTLAVPYNMRVIHFG | M5;M70 | ENMTPRDLGACIDCS | M64;M67;M14;M64;M67; |
| | AGSDKGVAP | | ARHINAQVAK | M14 |
| 256 | TLAVPYNMRVIHFGA | M5;M70 | NKVENMTPRDLGACI | M64;M67;M14;M64;M67; |
| | GSDKGVAPGT | | DCSARHINAQ | M14 |
| 257 | QYLNTLTLAVPYNMR | M5;M70 | NMTPRDLGACIDCSA | M64;M67;M14;M64;M67; |
| | VIHFGAGSDK | | RHINAQVAKS | M14 |
| 258 | YLNTLTLAVPYNMRVI | M5;M70 | KVENMTPRDLGACID | M64;M67;M14;M64;M67; |
| | HFGAGSDKG | | CSARHINAQV | M14 |
| 259 | LTLAVPYNMRVIHFGA | M5;M70 | TPRDLGACIDCSARHI | M64;M67;M14;M64;M67; |
| | GSDKGVAPG | | NAQVAKSHN | M14 |
| 260 | GSDKGVAPGTAVLRQ | M33;M70 | TYNKVENMTPRDLG | M64;M67;M14;M64;M67; |
| | WLPTGTLLVD | | ACIDCSARHIN | M14 |
| 261 | DKGVAPGTAVLRQWL | M33;M70 | YNKVENMTPRDLGA | M64;M67;M14;M64;M67; |
| | PTGTLLVDSD | | CIDCSARHINA | M14 |
| 262 | SDKGVAPGTAVLRQW | M33;M70 | VENMTPRDLGACIDC | M64;M67;M14;M64;M67; |
| | LPTGTLLVDS | | SARHINAQVA | M14 |
| 263 | KGVAPGTAVLRQWLP | M33;M70 | MTPRDLGACIDCSAR | M64;M67;M14;M64;M67; |
| | TGTLLVDSDL | | HINAQVAKSH | M14 |
| 264 | LSFELLHAPATVCGPK | M46;M27 | QRNFYEPQIITTDNTF | M64;M10;M7;M30;M9;M23 |
| | KSTNLVKNK | | VSGNCDVVI | |
| 265 | PAFDKSAFVNLKQLPF | M60;M54 | TQRNFYEPQIITTDNT | M64;M10;M7;M30;M9;M23 |
| | FYYSDSPCE | | FVSGNCDVV | |
| 266 | PLVSSQCVNLTTRTQL | M9;M24 | DTFNGECPNFVFPLNS | M50;M19;M61;M50;M19; |
| | PPAYTNSFT | | IIKTIQPRV | M61 |
| 267 | LLPLVSSQCVNLTTRT | M9;M24 | TFNGECPNFVFPLNSII | M50;M19;M61;M50;M19; |
| | QLPPAYTNS | | KTIQPRVE | M61 |
| 268 | LPLVSSQCVNLTTRTQ | M9;M24 | LEILQKEKVNINIVGD | M35;M27;M44;M35;M27; |
| | LPPAYTNSF | | FKLNEEIAI | M44 |
| 269 | GTDYKHWPQIAQFAPS | M82;M70 | EILQKEKVNINIVGDF | M35;M27;M44;M35;M27; |
| | ASAFFGMSR | | KLNEEIAII | M44 |
| 270 | RIIMRLWLCWKCRSK | M75;M76 | LLEILQKEKVNINIVG | M35;M27;M44;M35;M27; |
| | NPLLYDANYF | | DFKLNEEIA | M44 |
| 271 | VRIIMRLWLCWKCRS | M75;M76 | YTPSFKKGAKLLHKPI | M1;M27;M16;M1;M27;M16 |
| | KNPLLYDANY | | VWHVNNATN | |
| 272 | FVRIIMRLWLCWKCRS | M75;M76 | SDDAVVCFNSTYASQ | M13;M21;M53;M48;M49; |
| | KNPLLYDAN | | GLVASIKNFK | M22 |
| 273 | IIMRLWLCWKCRSKNP | M75;M76 | LSDDAVVCFNSTYAS | M13;M21;M53;M48;M49; |
| | LLYDANYFL | | QGLVASIKNF | M22 |
| 274 | ESGVKDCVVLHSYFTS | M46;M20 | LLSVLLSMQGAVDIN | M67;M45;M49;M67;M45; |
| | DYYQLYSTQ | | KLCEEMLDNR | M49 |
| 275 | GVITHDVSSAINRPQIG | M21;M70 | SLLSVLLSMQGAVDI | M67;M45;M49;M67;M45; |
| | VVREFLTR | | NKLCEEMLDN | M49 |
| 276 | GMSRIGMEVTPSGTW | M5;M23 | KLIEYTDFATSACVLA | M29;M14;M62;M29;M14; |
| | LTYTGAIKLD | | AECTIFKDA | M62 |
| 277 | FGMSRIGMEVTPSGT | M5;M23 | IEYTDFATSACVLAAE | M29;M14;M62;M29;M14; |
| | WLTYTGAIKL | | CTIFKDASG | M62 |
| 278 | LVAEWFLAYILFTRFF | M5;M5 | LIEYTDFATSACVLAA | M29;M14;M62;M29;M14; |
| | YVLGLAAIM | | ECTIFKDAS | M62 |
| 279 | LALHRSYLTPGDSSSG | M5;M75 | SKLIEYTDFATSACVL | M29;M14;M62;M29;M14; |
| | WTAGAAAYY | | AAECTIFKD | M62 |
| 280 | QNSVRVLQKAAITILD | M5;M5 | SRVSAKPPPGDQFKH | M54;M16;M60;M34;M52; |
| | GISQYSLRL | | LIPLMYKGLP | M39 |
| 281 | FGLVAEWFLAYILFTR | M5;M5 | GGSCVLSGHNLAKHC | M1;M80;M37;M1;M80;M37 |
| | FFYVLGLAA | | LHVVGPNVNK | |
| 282 | ALHRSYLTPGDSSSGW | M5;M75 | VGGSCVLSGHNLAKH | M1;M80;M37;M1;M80;M37 |
| | TAGAAAYYV | | CLHVVGPNVN | |
| 283 | GLVAEWFLAYILFTRF | M5;M5 | EVGPEHSLAEYHNES | M13;M39;M48;M13;M39; |
| | FYVLGLAAI | | GLKTILRKGG | M48 |
| 284 | LLALHRSYLTPGDSSS | M5;M75 | VGPEHSLAEYHNESG | M13;M39;M48;M13;M39; |
| | GWTAGAAAY | | LKTILRKGGR | M48 |
| 285 | HFGAGSDKGVAPGTA | M13;M26 | LHNWNCVNCDTFCA | M57;M45;M70;M57;M45; |
| | VLRQWLPTGT | | GSTFISDEVAR | M70 |
| 286 | GAGSDKGVAPGTAVL | M13;M26 | NSYTTTIKPVTYKLDG | M36;M59;M20;M36;M59; |
| | RQWLPTGTLL | | VVCTEIDPK | M20 |
| 287 | FGAGSDKGVAPGTAV | M13;M26 | KSAPLIELCVDEAGSK | M64;M40;M21;M7;M22; |
| | LRQWLPTGTL | | SPIQYIDIG | M23 |
| 288 | IHFGAGSDKGVAPGTA | M13;M26 | SAPLIELCVDEAGSKS | M64;M40;M21;M7;M22; |
| | VLRQWLPTG | | PIQYIDIGN | M23 |
| 289 | VIHFGAGSDKGVAPGT | M13;M26 | GARKSAPLIELCVDEA | M64;M40;M21;M7;M22; |
| | AVLRQWLPT | | GSKSPIQYI | M23 |
| 290 | AHKDKSAQCFKMFYK | M13;M52 | RKSAPLIELCVDEAGS | M64;M40;M21;M7;M22; |
| | GVITHDVSSA | | KSPIQYIDI | M23 |
| 291 | KAHKDKSAQCFKMFY | M13;M52 | ARKSAPLIELCVDEAG | M64;M40;M21;M7;M22; |
| | KGVITHDVSS | | SKSPIQYID | M23 |
| 292 | HKDKSAQCFKMFYKG | M13;M52 | VSFSTFEEAALCTFLL | M13;M44;M49;M13;M44; |
| | VITHDVSSAI | | NKEMYLKLR | M49 |
| 293 | LKAHKDKSAQCFKMF | M13;M52 | GAISSVLNDILSRLDK | M58;M53;M48;M50;M17; |
| | YKGVITHDVS | | VEAEVQIDR | M15 |
| 294 | KLKAHKDKSAQCFKM | M13;M52 | VNINIVGDFKLNEEIAI | M35;M73;M44;M35;M73; |
| | FYKGVITHDV | | ILASFSAS | M44 |
| 295 | EQIDGYVMHANYIFW | M41;M9 | QQTVTLLPAADLDDF | M54;M58;M55;M53;M26; |
| | RNTNPIQLSS | | SKQLQQSMSS | M39 |
| 296 | KPREQIDGYVMHANYI | M41;M9 | KQQTVTLLPAADLDD | M54;M58;M55;M53;M26; |
| | FWRNTNPIQ | | FSKQLQQSMS | M39 |
| 297 | PREQIDGYVMHANYIF | M41;M9 | LMWLSYFIASFRLFAR | M64;M40;M16;M30;M15; |
| | WRNTNPIQL | | TRSMWSFNP | M35 |
| 298 | REQIDGYVMHANYIF | M41;M9 | LPNNTASWFTALTQH | M40;M28;M70;M29;M18; |
| | WRNTNPIQLS | | GKEDLKFPRG | M15 |
| 299 | DYDYYRYNLPTMCDI | M74;M20 | GLPNNTASWFTALTQ | M40;M28;M70;M29;M18; |
| | RQLLFVVEVV | | HGKEDLKFPR | M15 |
| 300 | YDYYRYNLPTMCDIR | M74;M20 | QGLPNNTASWFTALT | M40;M28;M70;M29;M18; |
| | QLLFVVEVVD | | QHGKEDLKFP | M15 |
| 301 | YFVVKRHTFSNYQHE | M34;M20 | LVRDLPQGFSALEPLV | M31;M60;M34;M45;M52; |
| | ETIYNLLKDC | | DLPIGINIT | M47 |
| 302 | FVVKRHTFSNYQHEET | M34;M20 | RDLPQGFSALEPLVDL | M31;M60;M34;M45;M52; |
| | IYNLLKDCP | | PIGINITRF | M47 |
| 303 | SYFVVKRHTFSNYQHE | M34;M20 | NLVRDLPQGFSALEPL | M31;M60;M34;M45;M52; |
| | ETIYNLLKD | | VDLPIGINI | M47 |
| 304 | FDKAGQKTYERHSLS | M70;M70 | VRDLPQGFSALEPLV | M31;M60;M34;M45;M52; |
| | HFVNLDNLRA | | DLPIGINITR | M47 |
| 305 | NMRVIHFGAGSDKGV | M13;M70 | LALYNKYKYFSGAM | M1;M67;M66;M1;M67;M66 |
| | APGTAVLRQW | | DTTSYREAACC | |
| 306 | MRVIHFGAGSDKGVA | M13;M70 | KYKFVRIQPGQTFSVL | M48;M27;M16;M48;M27; |
| | PGTAVLRQWL | | ACYNGSPSG | M16 |
| 307 | YFDKAGQKTYERHSL | M70;M70 | RIQPGQTFSVLACYN | M48;M27;M16;M48;M27; |
| | SHFVNLDNLR | | GSPSGVYQCA | M16 |
| 308 | LIRQGTDYKHWPQIAQ | M69;M82 | VRIQPGQTFSVLACY | M48;M27;M16;M48;M27; |
| | FAPSASAFF | | NGSPSGVYQC | M16 |
| 309 | VESSSKLWAQCVQLH | M82;M82 | FVRIQPGQTFSVLACY | M48;M27;M16;M48;M27; |
| | NDILLAKDTT | | NGSPSGVYQ | M16 |
| 310 | LRVESSSKLWAQCVQ | M82;M82 | QPGQTFSVLACYNGS | M48;M27;M16;M48;M27; |
| | LHNDILLAKD | | PSGVYQCAMR | M16 |
| 311 | QLRVESSSKLWAQCV | M82;M82 | YKFVRIQPGQTFSVLA | M48;M27;M16;M48;M27; |
| | QLHNDILLAK | | CYNGSPSGV | M16 |
| 312 | TISSFKWDLTAFGLVA | M82;M82 | KFVRIQPGQTFSVLAC | M48;M27;M16;M48;M27; |
| | EWFLAYILF | | YNGSPSGVY | M16 |
| 313 | ESSSKLWAQCVQLHN | M82;M82 | IQPGQTFSVLACYNGS | M48;M27;M16;M48;M27; |
| | DILLAKDTTE | | PSGVYQCAM | M16 |
| 314 | ITISSFKWDLTAFGLVA | M82;M82 | KGVHFVCNLLLLFVT | M21;M6;M50;M43;M22; |
| | EWFLAYIL | | VYSHLLLVAA | M23 |
| 315 | DQELIRQGTDYKHWP | M69;M82 | FVCNLLLLFVTVYSH | M6;M37;M41;M43;M42; |
| | QIAQFAPSAS | | LLLVAAGLEA | M22 |
| 316 | RVESSSKLWAQCVQL | M82;M82 | CNLLLLFVTVYSHLLL | M6;M37;M41;M43;M42; |
| | HNDILLAKDT | | VAAGLEAPF | M22 |
| 317 | ISSFKWDLTAFGLVAE | M82;M82 | VCNLLLLFVTVYSHL | M6;M37;M41;M43;M42; |
| | WFLAYILFT | | LLVAAGLEAP | M22 |
| 318 | ELIRQGTDYKHWPQIA | M69;M82 | CGSVGFNIDYDCVSF | M43;M17;M14;M43;M17; |
| | QFAPSASAF | | CYMHHMELPT | M14 |
| 319 | QELIRQGTDYKHWPQI | M69;M82 | STECSNLLLQYGSFCT | M13;M48;M41;M43;M49; |
| | AQFAPSASA | | QLNRALTGI | M46 |
| 320 | ETYFTQSRNLQEFKPR | M75;M31 | DSTECSNLLLQYGSFC | M13;M48;M41;M43;M49; |
| | SQMEIDFLE | | TQLNRALTG | M46 |
| 321 | TYFTQSRNLQEFKPRS | M75;M31 | GDSTECSNLLLQYGSF | M13;M48;M41;M43;M49; |
| | QMEIDFLEL | | CTQLNRALT | M46 |
| 322 | YLQYIRKLHDELTGH | M32;M38 | GTITVEELKKLLEQW | M4;M58;M25;M53;M17; |
| | MLDMYSVMLT | | NLVIGFLFLT | M47 |
| 323 | CALDPLSETKCTLKSF | M75;M21 | CASYQTQTNSPRRAR | M31;M5;M25;M6;M50;M18 |
| | TVEKGIYQT | | SVASQSIIAY | |
| 324 | TLATCELYHYQECVR | M27;M20 | QPRVEKKKLDGFMG | M57;M26;M6;M57;M26;M6 |
| | GTTVLLKEPC | | RIRSVYPVASP | |
| 325 | LATCELYHYQECVRG | M27;M20 | RFASVYAWNRKRISN | M16;M55;M53;M74;M67; |
| | TTVLLKEPCS | | CVADYSVLYN | M27 |
| 326 | AITRAKVGILCIMSDR | M32;M68 | FASVYAWNRKRISNC | M16;M55;M53;M74;M67; |
| | DLYDKLQFT | | VADYSVLYNS | M27 |
| 327 | TRAKVGILCIMSDRDL | M32;M68 | LPNDDTLRVEAFEYY | M29;M6;M41;M29;M6;M41 |
| | YDKLQFTSL | | HTTDPSFLGR | |
| 328 | AKVGILCIMSDRDLYD | M32;M68 | PNDDTLRVEAFEYYH | M29;M6;M41;M29;M6;M41 |
| | KLQFTSLEI | | TTDPSFLGRY | |
| 329 | RAKVGILCIMSDRDLY | M32;M68 | SVVNIQKEIDRLNEVA | M78;M65;M2;M61;M79; |
| | DKLQFTSLE | | KNLNESLID | M82 |
| 330 | ITRAKVGILCIMSDRDL | M32;M68 | LSRVLGLKTLATHGL | M22;M6;M54;M22;M6;M54 |
| | YDKLQFTS | | AAVNSVPWDT | |
| 331 | KLASHMYCSFYPPDED | M20;M20 | IWFLLLSVCLGSLIYS | M29;M6;M31;M29;M6;M31 |
| | EEEGDCEEE | | TAALGVLMS | |
| 332 | ENFNQHEVLLAPLLSA | M20;M20 | KGIMMNVAKYTQLC | M73;M78;M34;M48;M41; |
| | GIFGADPIH | | QYLNTLTLAVP | M49 |
| 333 | NLAKHCLHVVGPNVN | M77;M77 | PKGIMMNVAKYTQL | M73;M78;M34;M48;M41; |
| | KGEDIQLLKS | | CQYLNTLTLAV | M49 |
| 334 | TWLDMVDTSLSGFKL | M75;M75 | TGCVIAWNSNNLDSK | M32;M48;M50;M68;M49; |
| | KDCVMYASAV | | VGGNYNYLYR | M26 |
| 335 | CHNKCAYWVPRASAN | M75;M75 | FTGCVIAWNSNNLDS | M32;M48;M50;M68;M49; |
| | IGCNHTGVVG | | KVGGNYNYLY | M26 |
| 336 | EFKLASHMYCSFYPPD | M20;M20 | NIDGYFKIYSKHTPIN | M64;M40;M71;M16;M15; |
| | EDEEEGDCE | | LVRDLPQGF | M52 |
| 337 | VGCHNKCAYWVPRAS | M75;M75 | SDVGDSAEVAVKMF | M26;M14;M37;M26;M14; |
| | ANIGCNHTGV | | DAYVNTFSSTF | M37 |
| 338 | NFNQHEVLLAPLLSAG | M20;M20 | VSDVGDSAEVAVKM | M26;M14;M37;M26;M14; |
| | IFGADPIHS | | FDAYVNTFSST | M37 |
| 339 | YVGCHNKCAYWVPR | M75;M75 | LNQLTGYKKPASREL | M57;M63;M70;M57;M63; |
| | ASANIGCNHTG | | KVTFFPDLNG | M70 |
| 340 | LAKHCLHVVGPNVNK | M77;M77 | VGTPVCINGLMLLEIK | M14;M9;M61;M14;M9;M61 |
| | GEDIQLLKSA | | DTEKYCALA | |
| 341 | AKHCLHVVGPNVNKG | M77;M77 | PLVGTPVCINGLMLLE | M14;M9;M61;M14;M9;M61 |
| | EDIQLLKSAY | | IKDTEKYCA | |
| 342 | FKLASHMYCSFYPPDE | M20;M20 | GTPVCINGLMLLEIKD | M14;M9;M61;M14;M9;M61 |
| | DEEEGDCEE | | TEKYCALAP | |
| 343 | GCHNKCAYWVPRASA | M75;M75 | LVGTPVCINGLMLLEI | M14;M9;M61;M14;M9;M61 |
| | NIGCNHTGVV | | KDTEKYCAL | |
| 344 | YEGNSPFHPLADNKFA | M1;M3;M70 | TPVCINGLMLLEIKDT | M14;M9;M61;M14;M9;M61 |
| | LTCFSTQFA | | EKYCALAPN | |
| 345 | TYEGNSPFHPLADNKF | M1;M3;M70 | TDDNALAYYNTTKG | M21;M22;M6;M21;M22;M6 |
| | ALTCFSTQF | | GRFVLALLSDL | |
| 346 | EIRASANLAATKMSEC | M4;M27;M3 | MGRIRSVYPVASPNE | M68;M29;M6;M68;M29;M6 |
| | VLGQSKRVD | | CNQMCLSTLM | |
| 347 | IRASANLAATKMSECV | M4;M27;M3 | QGAVDINKLCEEMLD | M21;M45;M49;M21;M45; |
| | LGQSKRVDF | | NRATLQAIAS | M49 |
| 348 | DGTLMIERFVSLAIDA | M5;M2;M3 | GAVDINKLCEEMLDN | M21;M45;M49;M21;M45; |
| | YPLTKHPNQ | | RATLQAIASE | M49 |
| 349 | GTLMIERFVSLAIDAY | M5;M2;M3 | DMVDTSLSGFKLKDC | M15;M75;M14;M15;M75; |
| | PLTKHPNQE | | VMYASAVVLL | M14 |
| 350 | SGTYEGNSPFHPLADN | M3;M44;M70 | WLDMVDTSLSGFKLK | M15;M75;M14;M15;M75; |
| | KFALTCFST | | DCVMYASAVV | M14 |
| 351 | VIGTSKFYGGWHNML | M18;M3;M6 | MVDTSLSGFKLKDCV | M15;M75;M14;M15;M75; |
| | KTVYSDVENP | | MYASAVVLLI | M14 |
| 352 | CRSKNPLLYDANYFLC | M18;M21; | LDMVDTSLSGFKLKD | M15;M75;M14;M15;M75; |
| | WHTNCYDYC | M3 | CVMYASAVVL | M14 |
| 353 | RSKNPLLYDANYFLC | M18;M21; | AGTTQTACTDDNALA | M22;M27;M6;M22;M27;M6 |
| | WHTNCYDYCI | M3 | YYNTTKGGRF | |
| 354 | GVKDCVVLHSYFTSD | M42;M3; | STQYEYGTEDDYQGK | M1;M67;M37;M1;M67;M37 |
| | YYQLYSTQLS | M20 | PLEFGATSAA | |
| 355 | VEIIKSQDLSVVSKVV | M51;M1; | PSTQYEYGTEDDYQG | M1;M67;M37;M1;M67;M37 |
| | KVTIDYTEI | M18 | KPLEFGATSA | |
| 356 | KSQDLSVVSKVVKVTI | M51;M1; | EPSTQYEYGTEDDYQ | M1;M67;M37;M1;M67;M37 |
| | DYTEISFML | M18 | GKPLEFGATS | |
| 357 | IIKSQDLSVVSKVVKV | M51;M1; | QYEYGTEDDYQGKPL | M1;M22;M37;M1;M22;M37 |
| | TIDYTEISF | M18 | EFGATSAALQ | |
| 358 | IKSQDLSVVSKVVKVT | M51;M1; | EYGTEDDYQGKPLEF | M1;M22;M37;M1;M22;M37 |
| | IDYTEISFM | M18 | GATSAALQPE | |
| 359 | EIIKSQDLSVVSKVVK | M51;M1; | YEYGTEDDYQGKPLE | M1;M22;M37;M1;M22;M37 |
| | VTIDYTEIS | M18 | FGATSAALQP | |
| 360 | FVEIIKSQDLSVVSKVV | M51;M1; | LKLKVDTANPKTPKY | M77;M34;M14;M77;M34; |
| | KVTIDYTE | M18 | KFVRIQPGQT | M14 |
| 361 | YVIFTQTTETAHSCNV | M51;M6; | CTLKSFTVEKGIYQTS | M75;M25;M37;M41;M8; |
| | NRFNVAITR | M59 | NFRVQPTES | M23 |
| 362 | DYVIFTQTTETAHSCN | M51;M6; | AVIKTLQPVSELLTPL | M78;M45;M49;M78;M45; |
| | VNRFNVAIT | M59 | GIDLDEWSM | M49 |
| 363 | AQTGSSKCVCSVIDLL | M4;M51; | VIKTLQPVSELLTPLGI | M78;M45;M49;M78;M45; |
| | LDDFVEIIK | M59 | DLDEWSMA | M49 |
| 364 | QTGSSKCVCSVIDLLL | M4;M51; | PRHVICTSEDMLNPN | M72;M28;M59;M72;M28; |
| | DDFVEIIKS | M59 | YEDLLIRKSN | M59 |
| 365 | LNDFVSDADSTLIGDC | M7;M6; | AGTDTTITVNVLAWL | M56;M32;M14;M56;M32; |
| | ATVHTANKW | M36 | YAAVINGDRW | M14 |
| 366 | DLNDFVSDADSTLIGD | M7;M6; | DVVNQNAQALNTLV | M58;M16;M53;M17;M15; |
| | CATVHTANK | M36 | KQLSSNFGAIS | M22 |
| 367 | MPLSAPTLVPQEHYVR | M51;M69; | VVNQNAQALNTLVK | M58;M16;M53;M17;M15; |
| | ITGLYPTLN | M28 | QLSSNFGAISS | M22 |
| 368 | TVMPLSAPTLVPQEHY | M51;M69; | CDIPIGAGICASYQTQ | M28;M31;M70;M25;M50; |
| | VRITGLYPT | M28 | TNSPRRARS | M18 |
| 369 | HTVMPLSAPTLVPQEH | M51;M69; | ECDIPIGAGICASYQT | M28;M31;M70;M25;M50; |
| | YVRITGLYP | M28 | QTNSPRRAR | M18 |
| 370 | VMPLSAPTLVPQEHYV | M51;M69; | YECDIPIGAGICASYQ | M28;M31;M70;M25;M50; |
| | RITGLYPTL | M28 | TQTNSPRRA | M18 |
| 371 | NHTSPDVDLGDISGIN | M14;M80; | EVAVKMFDAYVNTFS | M41;M8;M70;M41;M8;M70 |
| | ASVVNIQKE | M45 | STFNVPMEKL | |
| 372 | SKVVKVTIDYTEISFM | M71;M67; | VAVKMFDAYVNTFSS | M41;M8;M70;M41;M8;M70 |
| | LWCKDGHVE | M51 | TFNVPMEKLK | |
| 373 | ANTVIWDYKRDAPAH | M36;M11; | GYLPQNAVVKIYCPA | M35;M11;M28;M35;M11; |
| | ISTIGVCSMT | M6 | CHNSEVGPEH | M28 |
| 374 | TVIWDYKRDAPAHIST | M36;M11; | EVPVAIHADQLTPTW | M58;M24;M16;M5;M41; |
| | IGVCSMTDI | M6 | RVYSTGSNVF | M17 |
| 375 | NTVIWDYKRDAPAHIS | M36;M11; | TEVPVAIHADQLTPT | M58;M24;M16;M5;M41; |
| | TIGVCSMTD | M6 | WRVYSTGSNV | M17 |
| 376 | VIWDYKRDAPAHISTI | M36;M11; | CTEVPVAIHADQLTPT | M58;M24;M16;M5;M41; |
| | GVCSMTDIA | M6 | WRVYSTGSN | M17 |
| 377 | MYSVMLTNDNTSRY | M7;M22; | QKVGMQKYSTLQGPP | M34;M29;M41;M42;M43; |
| | WEPEFYEAMYT | M36 | GTGKSHFAIG | M39 |
| 378 | YSVMLTNDNTSRYWE | M7;M22; | KVGMQKYSTLQGPPG | M34;M29;M41;M42;M43; |
| | PEFYEAMYTP | M36 | TGKSHFAIGL | M39 |
| 379 | YFDCYDGGCINANQVI | M50;M73; | VDADSKIVQLSEISMD | M7;M54;M49;M7;M54;M49 |
| | VNNLDKSAG | M51 | NSPNLAWPL | |
| 380 | FDCYDGGCINANQVIV | M50;M73; | GEAANFCALILAYCN | M41;M48;M49;M41;M48; |
| | NNLDKSAGF | M51 | KTVGELGDVR | M49 |
| 381 | DCYDGGCINANQVIV | M50;M73; | RAGEAANFCALILAY | M41;M48;M49;M41;M48; |
| | NNLDKSAGFP | M51 | CNKTVGELGD | M49 |
| 382 | SAPTLVPQEHYVRITG | M51;M69; | AGEAANFCALILAYC | M41;M48;M49;M41;M48; |
| | LYPTLNISD | M34 | NKTVGELGDV | M49 |
| 383 | APTLVPQEHYVRITGL | M51;M69; | LVATAEAELAKNVSL | M7;M29;M73;M7;M29;M73 |
| | YPTLNISDE | M34 | DNVLSTFISA | |
| 384 | PTLVPQEHYVRITGLY | M51;M69; | VATAEAELAKNVSLD | M7;M29;M73;M7;M29;M73 |
| | PTLNISDEF | M34 | NVLSTFISAA | |
| 385 | LSAPTLVPQEHYVRIT | M51;M69; | PGVYSVIYLYLTFYLT | M50;M48;M49;M50;M48; |
| | GLYPTLNIS | M34 | NDVSFLAHI | M49 |
| 386 | PTMCDIRQLLFVVEVV | M51;M70; | LPGVYSVIYLYLTFYL | M50;M48;M49;M50;M48; |
| | DKYFDCYDG | M20 | TNDVSFLAH | M49 |
| 387 | IRQLLFVVEVVDKYFD | M51;M70; | NFCALILAYCNKTVG | M79;M41;M48;M79;M41; |
| | CYDGGCINA | M37 | ELGDVRETMS | M48 |
| 388 | NLPTMCDIRQLLFVVE | M51;M70; | CALILAYCNKTVGEL | M79;M41;M48;M79;M41; |
| | VVDKYFDCY | M20 | GDVRETMSYL | M48 |
| 389 | LPTMCDIRQLLFVVEV | M51;M70; | FCALILAYCNKTVGE | M79;M41;M48;M79;M41; |
| | VDKYFDCYD | M20 | LGDVRETMSY | M48 |
| 390 | RYNLPTMCDIRQLLFV | M51;M70; | KSNHNFLVQAGNVQL | M12;M58;M70;M12;M58; |
| | VEVVDKYFD | M20 | RVIGHSMQNC | M70 |
| 391 | DIRQLLFVVEVVDKYF | M51;M70; | RKSNHNFLVQAGNV | M12;M58;M70;M12;M58; |
| | DCYDGGCIN | M37 | QLRVIGHSMQN | M70 |
| 392 | YNLPTMCDIRQLLFVV | M51;M70; | RYFRLTLGVYDYLVS | M43;M6;M31;M43;M6;M31 |
| | EVVDKYFDC | M20 | TQEFRYMNSQ | |
| 393 | VDCTMYICGDSTECSN | M48;M39; | LMSFPQSAPHGVVFL | M64;M21;M28;M70;M53; |
| | LLLQYGSFC | M45 | HVTYVPAQEK | M9 |
| 394 | DCTMYICGDSTECSNL | M48;M39; | NALAYYNTTKGGRFV | M33;M6;M20;M33;M6;M20 |
| | LLQYGSFCT | M45 | LALLSDLQDL | |
| 395 | EQTQGNFGDQELIRQG | M64;M69; | YQGKPLEFGATSAAL | M38;M22;M28;M38;M22; |
| | TDYKHWPQI | M51 | QPEEEQEEDW | M28 |
| 396 | QGNFGDQELIRQGTDY | M64;M69; | KPLEFGATSAALQPEE | M38;M22;M28;M38;M22; |
| | KHWPQIAQF | M51 | EQEEDWLDD | M28 |
| 397 | TQGNFGDQELIRQGTD | M64;M69; | DYQGKPLEFGATSAA | M38;M22;M28;M38;M22; |
| | YKHWPQIAQ | M51 | LQPEEEQEED | M28 |
| 398 | PEQTQGNFGDQELIRQ | M64;M69; | DDYQGKPLEFGATSA | M38;M22;M28;M38;M22; |
| | GTDYKHWPQ | M51 | ALQPEEEQEE | M28 |
| 399 | QTQGNFGDQELIRQGT | M64;M69; | QGKPLEFGATSAALQ | M38;M22;M28;M38;M22; |
| | DYKHWPQIA | M51 | PEEEQEEDWL | M28 |
| 400 | GNFGDQELIRQGTDYK | M64;M69; | GKPLEFGATSAALQP | M38;M22;M28;M38;M22; |
| | HWPQIAQFA | M51 | EEEQEEDWLD | M28 |
| 401 | FGDQELIRQGTDYKH | M64;M69; | VMFTPLVPFWITIAYII | M60;M56;M8;M60;M56;M8 |
| | WPQIAQFAPS | M51 | CISTKHFY | |
| 402 | NFGDQELIRQGTDYKH | M64;M69; | KGGKIVNNWLKQLIK | M57;M55;M11;M57;M55; |
| | WPQIAQFAP | M51 | VTLVFLFVAA | M11 |
| 403 | FVSDADSTLIGDCATV | M7;M24; | FKLKDCVMYASAVV | M33;M75;M12;M33;M75; |
| | HTANKWDLI | M36 | LLILMTARTVY | M12 |
| 404 | VSDADSTLIGDCATVH | M7;M24; | PQTSITSAVLQSGFRK | M1;M38;M62;M1;M38;M62 |
| | TANKWDLII | M36 | MAFPSGKVE | |
| 405 | DFVSDADSTLIGDCAT | M7;M24; | QTSITSAVLQSGFRKM | M1;M38;M62;M1;M38;M62 |
| | VHTANKWDL | M36 | AFPSGKVEG | |
| 406 | LPDPSKPSKRSFIEDLL | M63;M67; | LNRFTTTLNDFNLVA | M63;M73;M66;M63;M73; |
| | FNKVTLAD | M44 | MKYNYEPLTQ | M66 |
| 407 | KLLKSIAATRGATVVI | M72;M2; | PINVIVFDGKSKCEES | M7;M56;M52;M7;M56;M52 |
| | GTSKFYGGW | M14 | SAKSASVYY | |
| 408 | PGDQFKHLIPLMYKGL | M1;M39; | QLSLPVLQVRDVLVR | M43;M63;M70;M43;M63; |
| | PWNVVRIKI | M12 | GFGDSVEEVL | M70 |
| 409 | GDQFKHLIPLMYKGLP | M1;M39; | HVQLSLPVLQVRDVL | M43;M63;M70;M43;M63; |
| | WNVVRIKIV | M12 | VRGFGDSVEE | M70 |
| 410 | SLFDMSKFPLKLRGTA | M67;M14; | VQLSLPVLQVRDVLV | M43;M63;M70;M43;M63; |
| | VMSLKEGQI | M8 | RGFGDSVEEV | M70 |
| 411 | AETLKATEETFKLSYG | M33;M6; | LSLPVLQVRDVLVRG | M43;M63;M70;M43;M63; |
| | IATVREVLS | M44 | FGDSVEEVLS | M70 |
| 412 | ETLKATEETFKLSYGI | M33;M6; | EDDYQGKPLEFGATS | M22;M28;M37;M22;M28; |
| | ATVREVLSD | M44 | AALQPEEEQE | M37 |
| 413 | VQELYSPIFLIVAAIVFI | M25;M12; | TEDDYQGKPLEFGAT | M22;M28;M37;M22;M28; |
| | TLCFTLK | M14 | SAALQPEEEQ | M37 |
| 414 | ELYSPIFLIVAAIVFITL | M25;M12; | YLFQHANLDSCKRVL | M5;M27;M2;M5;M27;M2 |
| | CFTLKRK | M14 | NVVCKTCGQQ | |
| 415 | EVQELYSPIFLIVAAIV | M25;M12; | IDRLITGRLQSLQTYV | M34;M29;M38;M49;M46; |
| | FITLCFTL | M14 | TQQLIRAAE | M22 |
| 416 | QELYSPIFLIVAAIVFIT | M25;M12; | VQIDRLITGRLQSLQT | M34;M29;M38;M49;M46; |
| | LCFTLKR | M14 | YVTQQLIRA | M22 |
| 417 | MMGFKMNYQVNGYP | M44;M48; | QIDRLITGRLQSLQTY | M34;M29;M38;M49;M46; |
| | NMFITREEAIR | M49 | VTQQLIRAA | M22 |
| 418 | MGFKMNYQVNGYPN | M44;M48; | EVQIDRLITGRLQSLQ | M34;M29;M38;M49;M46; |
| | MFITREEAIRH | M49 | TYVTQQLIR | M22 |
| 419 | ISMMGFKMNYQVNGY | M44;M48; | PKSDGTGTIYTELEPP | M38;M6;M59;M38;M6;M59 |
| | PNMFITREEA | M49 | CRFVTDTPK | |
| 420 | SMMGFKMNYQVNGY | M44;M48; | YCPIFFITGNTLQCIML | M40;M39;M11;M40;M39; |
| | PNMFITREEAI | M49 | VYCFLGYF | M11 |
| 421 | LISMMGFKMNYQVNG | M44;M48; | EYCPIFFITGNTLQCIM | M40;M39;M11;M40;M39; |
| | YPNMFITREE | M49 | LVYCFLGY | M11 |
| 422 | KGDYGDAVVYRGTTT | M9;M8; | IVFMCVEYCPIFFITGN | M40;M39;M11;M40;M39; |
| | YKLNVGDYFV | M44 | TLQCIMLV | M11 |
| 423 | DYGDAVVYRGTTTYK | M9;M8; | CVEYCPIFFITGNTLQ | M40;M39;M11;M40;M39; |
| | LNVGDYFVLT | M44 | CIMLVYCFL | M11 |
| 424 | YGDAVVYRGTTTYKL | M9;M8; | MCVEYCPIFFITGNTL | M40;M39;M11;M40;M39; |
| | NVGDYFVLTS | M44 | QCIMLVYCF | M11 |
| 425 | GDYGDAVVYRGTTTY | M9;M8; | VEYCPIFFITGNTLQCI | M40;M39;M11;M40;M39; |
| | KLNVGDYFVL | M44 | MLVYCFLG | M11 |
| 426 | SVELKHFFFAQDGNA | M36;M39; | FMCVEYCPIFFITGNT | M40;M39;M11;M40;M39; |
| | AISDYDYYRY | M67 | LQCIMLVYC | M11 |
| 427 | SSVELKHFFFAQDGNA | M36;M39; | VFMCVEYCPIFFITGN | M40;M39;M11;M40;M39; |
| | AISDYDYYR | M67 | TLQCIMLVY | M11 |
| 428 | VELKHFFFAQDGNAAI | M36;M39; | SDLQDLKWARFPKSD | M4;M60;M38;M4;M60;M38 |
| | SDYDYYRYN | M67 | GTGTIYTELE | |
| 429 | EGSSVELKHFFFAQDG | M36;M39; | PKGPKVKYLYFIKGL | M64;M52;M2;M64;M52;M2 |
| | NAAISDYDY | M67 | NNLNRGMVLG | |
| 430 | GSSVELKHFFFAQDGN | M36;M39; | DTPKGPKVKYLYFIK | M64;M52;M2;M64;M52;M2 |
| | AAISDYDYY | M67 | GLNNLNRGMV | |
| 431 | TFEKGDYGDAVVYRG | M9;M12; | TPKGPKVKYLYFIKG | M64;M52;M2;M64;M52;M2 |
| | TTTYKLNVGD | M44 | LNNLNRGMVL | |
| 432 | REPMLQSADAQSFLNR | M33;M12; | TDTPKGPKVKYLYFI | M64;M52;M2;M64;M52;M2 |
| | VCGVSAARL | M45 | KGLNNLNRGM | |
| 433 | YGCSCDQLREPMLQS | M33;M12; | VLSGHNLAKHCLHVV | M1;M77;M37;M1;M77;M37 |
| | ADAQSFLNRV | M45 | GPNVNKGEDI | |
| 434 | CSCDQLREPMLQSAD | M33;M12; | LSGHNLAKHCLHVVG | M1;M77;M37;M1;M77;M37 |
| | AQSFLNRVCG | M45 | PNVNKGEDIQ | |
| 435 | GYGCSCDQLREPMLQ | M33;M12; | RELKVTFFPDLNGDV | M60;M21;M63;M60;M21; |
| | SADAQSFLNG | M45 | VAIDYKHYTP | M63 |
| 436 | DQLREPMLQSADAQS | M33;M12; | VLQVRDVLVRGFGDS | M63;M68;M70;M63;M68; |
| | FLNRVCGVSA | M45 | VEEVLSEARQ | M70 |
| 437 | QLREPMLQSADAQSFL | M33;M12; | LQVRDVLVRGFGDSV | M63;M68;M70;M63;M68; |
| | NRVCGVSAA | M45 | EEVLSEARQH | M70 |
| 438 | CDQLREPMLQSADAQ | M33;M12; | QVRDVLVRGFGDSVE | M63;M68;M70;M63;M68; |
| | SFLNRVCGVS | M45 | EVLSEARQHL | M70 |
| 439 | GYGCSCDQLREPMLQ | M33;M12; | PVLQVRDVLVRGFGD | M63;M68;M70;M63;M68; |
| | SADAQSFLNR | M45 | SVEEVLSEAR | M70 |
| 440 | SCDQLREPMLQSADA | M33;M12; | RKHTTCCSLSHRFYR | M5;M60;M79;M43;M42; |
| | QSFLNRVCGV | M45 | LANECAQVLS | M39 |
| 441 | LREPMLQSADAQSFLN | M33;M12; | YQKVGMQKYSTLQG | M21;M34;M41;M42;M43; |
| | RVCGVSAAR | M45 | PPGTGKSHFAI | M39 |
| 442 | GCSCDQLREPMLQSA | M33;M12; | ANYQKVGMQKYSTL | M21;M34;M41;M42;M43; |
| | DAQSFLNRVC | M45 | QGPPGTGKSHF | M39 |
| 443 | IGPERTCCLCDRRATC | M4;M7; | NYQKVGMQKYSTLQ | M21;M34;M41;M42;M43; |
| | FSTASDTYA | M72 | GPPGTGKSHFA | M39 |
| 444 | GPERTCCLCDRRATCF | M4;M7; | VANYQKVGMQKYST | M21;M34;M41;M42;M43; |
| | STASDTYAC | M72 | LQGPPGTGKSH | M39 |
| 445 | FKNLREFVFKNIDGYF | M71;M19; | VRRSFYVYANGGKGF | M39;M11;M41;M39;M11; |
| | KIYSKHTPI | M16 | CKLHNWNCVN | M41 |
| 446 | VRFPNITNLCPFGEVFN | M45;M28; | AKNRARTVAGVSICS | M64;M32;M60;M30;M22; |
| | ATRFASVY | M66 | TMTNRQFHQK | M23 |
| 447 | IVRFPNITNLCPFGEVF | M45;M28; | VAGVSICSTMTNRQF | M64;M32;M60;M30;M22; |
| | NATRFASV | M66 | HQKLLKSIAA | M23 |
| 448 | ESIVRFPNITNLCPFGE | M45;M28; | AGVSICSTMTNRQFH | M64;M32;M60;M30;M22; |
| | VFNATRFA | M66 | QKLLKSIAAT | M23 |
| 449 | TESIVRFPNITNLCPFG | M45;M28; | ARTVAGVSICSTMTN | M64;M32;M60;M30;M22; |
| | EVFNATRF | M66 | RQFHQKLLKS | M23 |
| 450 | SIVRFPNITNLCPFGEV | M45;M28; | TVAGVSICSTMTNRQ | M64;M32;M60;M30;M22; |
| | FNATRFAS | M66 | FHQKLLKSIA | M23 |
| 451 | HQKLLKSIAATRGATV | M25;M2; | RARTVAGVSICSTMT | M64;M32;M60;M30;M22; |
| | VIGTSKFYG | M14 | NRQFHQKLLK | M23 |
| 452 | QKLLKSIAATRGATVV | M25;M2; | NRARTVAGVSICSTM | M64;M32;M60;M30;M22; |
| | IGTSKFYGG | M14 | TNRQFHQKLL | M23 |
| 453 | FHQKLLKSIAATRGAT | M25;M2; | RTVAGVSICSTMTNR | M64;M32;M60;M30;M22; |
| | VVIGTSKFY | M14 | QFHQKLLKSI | M23 |
| 454 | VTIDYTEISFMLWCKD | M71;M36; | KNRARTVAGVSICST | M64;M32;M60;M30;M22; |
| | GHVETFYPK | M67 | MTNRQFHQKL | M23 |
| 455 | TEISFMLWCKDGHVET | M71;M36; | SAKNRARTVAGVSIC | M64;M32;M60;M30;M22; |
| | FYPKLQSSQ | M67 | STMTNRQFHQ | M23 |
| 456 | VVKVTIDYTEISFMLW | M71;M36; | GHVMVELVAELEGIQ | M60;M54;M28;M60;M54; |
| | CKDGHVETF | M67 | YGRSGETLGV | M28 |
| 457 | IDYTEISFMLWCKDGH | M71;M36; | PHGHVMVELVAELEG | M60;M54;M28;M60;M54; |
| | VETFYPKLQ | M67 | IQYGRSGETL | M28 |
| 458 | YTEISFMLWCKDGHV | M71;M36; | RTAPHGHVMVELVA | M60;M54;M28;M60;M54; |
| | ETFYPKLQSS | M67 | ELEGIQYGRSG | M28 |
| 459 | VKVTIDYTEISFMLWC | M71;M36; | HGHVMVELVAELEGI | M60;M54;M28;M60;M54; |
| | KDGHVETFY | M67 | QYGRSGETLG | M28 |
| 460 | TIDYTEISFMLWCKDG | M71;M36; | TAPHGHVMVELVAEL | M60;M54;M28;M60;M54; |
| | HVETFYPKL | M67 | EGIQYGRSGE | M28 |
| 461 | KVTIDYTEISFMLWCK | M71;M36; | HVMVELVAELEGIQY | M60;M54;M28;M60;M54; |
| | DGHVETFYP | M67 | GRSGETLGVL | M28 |
| 462 | DYTEISFMLWCKDGH | M71;M36; | APHGHVMVELVAELE | M60;M54;M28;M60;M54; |
| | VETFYPKLQS | M67 | GIQYGRSGET | M28 |
| 463 | NSIAIPTNFTISVTTEIL | M33;M28; | SHGKQVVSDIDYVPL | M25;M34;M18;M43;M42; |
| | PVSMTKT | M45 | KSATCITRCN | M39 |
| 464 | AIPTNFTISVTTEILPVS | M33;M28; | KQVVSDIDYVPLKSA | M25;M34;M18;M43;M42; |
| | MTKTSVD | M45 | TCITRQNLGG | M39 |
| 465 | SNNSIAIPTNFTISVTTE | M33;M28; | GKQVVSDIDYVPLKS | M25;M34;M18;M43;M42; |
| | ILPVSMT | M45 | ATCITRQNLG | M39 |
| 466 | IPTNFTISVTTEILPVSM | M33;M28; | HGKQVVSDIDYVPLK | M25;M34;M18;M43;M42; |
| | TKTSVDC | M45 | SATCITRQNL | M39 |
| 467 | YSNNSIAIPTNFTISVTT | M33;M28; | VVSDIDYVPLKSATCI | M25;M34;M18;M43;M42; |
| | EILPVSM | M45 | TRCNLGGAV | M39 |
| 468 | IAIPTNFTISVTTEILPV | M33;M28; | QVVSDIDYVPLKSAT | M25;M34;M18;M43;M42; |
| | SMTKTSV | M45 | CITRQNLGGA | M39 |
| 469 | AYSNNSIAIPTNFTISV | M33;M28; | RFQTLLALHRSYLTPG | M21;M24;M70;M55;M5; |
| | TTEILPVS | M45 | DSSSGWTAG | M23 |
| 470 | SIAIPTNFTISVTTEILP | M33;M28; | FQTLLALHRSYLTPG | M21;M24;M70;M55;M5; |
| | VSMTKTS | M45 | DSSSGWTAGA | M23 |
| 471 | NNSIAIPTNFTISVTTEI | M33;M28; | IDKVLNEKCSAYTVE | M57;M43;M39;M57;M43; |
| | LPVSMTK | M45 | LGTEVNEFAC | M39 |
| 472 | VAYSNNSIAIPTNFTIS | M33;M28; | KVLNEKCSAYTVELG | M57;M43;M39;M57;M43; |
| | VTTEILPV | M45 | TEVNEFACVV | M39 |
| 473 | PKDMTYRRLISMMGF | M46;M39; | DKVLNEKCSAYTVEL | M57;M43;M39;M57;M43; |
| | KMNYQVNGYP | M44 | GTEVNEFACV | M39 |
| 474 | KDMTYRRLISMMGFK | M46;M39; | ERIDKVLNEKCSAYT | M57;M43;M39;M57;M43; |
| | MNYQVNGYPN | M44 | VELGTEVNEF | M39 |
| 475 | KCVPQADVEWKFYDA | M41;M6; | DERIDKVLNEKCSAY | M57;M43;M39;M57;M43; |
| | QPCSDKAYKI | M8 | TVELGTEVNE | M39 |
| 476 | MFHLVDFQVTIAEILLI | M4;M61; | VLNEKCSAYTVELGT | M57;M43;M39;M57;M43; |
| | IMRTFKVS | M8 | EVNEFACVVA | M39 |
| 477 | EETFKLSYGIATVREV | M12;M6; | RIDKVLNEKCSAYTV | M57;M43;M39;M57;M43; |
| | LSDRELHLS | M59 | ELGTEVNEFA | M39 |
| 478 | TFKLSYGIATVREVLS | M12;M6; | YFTSDYYQLYSTQLS | M40;M41;M43;M42;M39; |
| | DRELHLSWE | M59 | TDTGVEHVTF | M20 |
| 479 | FKLSYGIATVREVLSD | M12;M6; | LHSYFTSDYYQLYST | M40;M41;M43;M42;M39; |
| | RELHLSWEV | M59 | QLSTDTGVEH | M20 |
| 480 | ETFKLSYGIATVREVL | M12;M6; | VVLHSYFTSDYYQLY | M40;M41;M43;M42;M39; |
| | SDRELHLSW | M59 | STQLSTDTGV | M20 |
| 481 | KVDGVDVELFENKTT | M29;M28; | HSYFTSDYYQLYSTQ | M40;M41;M43;M42;M39; |
| | LPVNVAFELW | M48 | LSTDTGVEHV | M20 |
| 482 | DGVDVELFENKTTLPV | M29;M28; | SYFTSDYYQLYSTQLS | M40;M41;M43;M42;M39; |
| | NVAFELWAK | M48 | TDTGVEHVT | M20 |
| 483 | VDGVDVELFENKTTLP | M29;M28; | FTSDYYQLYSTQLST | M40;M41;M43;M42;M39; |
| | VNVAFELWA | M48 | DTGVEHVTFF | M20 |
| 484 | YQTQTNSPRRARSVAS | M5;M2;M6 | VLHSYFTSDYYQLYS | M40;M41;M43;M42;M39; |
| | QSIIAYTMS | | TQLSTDTGVE | M20 |
| 485 | SYQTQTNSPRRARSVA | M5;M2;M6 | ELWAKRNIKPVPEVKI | M54;M66;M74;M57;M49; |
| | SQSIIAYTM | | LNNLGVDIA | M9 |
| 486 | AKKPTETICAPLTVFFD | M7;M63; | KIQDSLSSTASALGKL | M40;M64;M29;M79;M22; |
| | GRVDGQVD | M49 | QDVVNQNAQ | M47 |
| 487 | IAKKPTETICAPLTVFF | M7;M63; | IGKIQDSLSSTASALG | M40;M64;M29;M79;M22; |
| | DGRVDGQV | M49 | KLQDVVNQN | M47 |
| 488 | KKPTETICAPLTVFFDG | M7;M63; | SAIGKIQDSLSSTASA | M40;M64;M29;M79;M22; |
| | RVDGQVDL | M49 | LGKLQDVVN | M47 |
| 489 | PTETICAPLTVFFDGRV | M7;M63; | IQDSLSSTASALGKLQ | M40;M64;M29;M79;M22; |
| | DGQVDLFR | M49 | DVVNQNAQA | M47 |
| 490 | TETICAPLTVFFDGRV | M7;M63; | AIGKIQDSLSSTASAL | M40;M64;M29;M79;M22; |
| | DGQVDLFRN | M49 | GKLQDVVNQ | M47 |
| 491 | KPTETICAPLTVFFDGR | M7;M63; | GKIQDSLSSTASALGK | M40;M64;M29;M79;M22; |
| | VDGQVDLF | M49 | LQDVVNQNA | M47 |
| 492 | YPANSIVCRFDTRVLS | M5;M2; | AARVVRSIFSRTLETA | M34;M2;M59;M34;M2;M59 |
| | NLNLPGCDG | M48 | QNSVRVLQK | |
| 493 | PANSIVCRFDTRVLSN | M5;M2; | NAVVKIYCPACHNSE | M68;M28;M35;M68;M28; |
| | LNLPGCDGG | M48 | VGPEHSLAEY | M35 |
| 494 | DGNAAISDYDYYRYN | M11;M74; | SIIKTIQPRVEKKKLD | M26;M29;M72;M26;M29; |
| | LPTMCDIRQL | M44 | GFMGRIRSV | M72 |
| 495 | ISDYDYYRYNLPTMC | M11;M74; | YLTSSSKTPEEHFIETI | M40;M29;M16;M40;M29; |
| | DIRQLLFVVE | M44 | SLAGSYKD | M16 |
| 496 | AISDYDYYRYNLPTM | M11;M74; | GEDIQLLKSAYENFN | M38;M60;M28;M38;M60; |
| | CDIRQLLFVV | M44 | QHEVLLAPLL | M28 |
| 497 | GNAAISDYDYYRYNL | M11;M74; | GIFGADPIHSLRVCVD | M68;M28;M31;M68;M28; |
| | PTMCDIRQLL | M44 | TVRTNVYLA | M31 |
| 498 | AAISDYDYYRYNLPT | M11;M74; | IFGADPIHSLRVCVDT | M68;M28;M31;M68;M28; |
| | MCDIRQLLFV | M44 | VRTNVYLAV | M31 |
| 499 | NAAISDYDYYRYNLPT | M11;M74; | YVGYLQPRTFLLKYN | M58;M24;M49;M15;M11; |
| | MCDIRQLLF | M44 | ENGTITDAVD | M47 |
| 500 | SGVPVVDSYYSLLMPI | M34;M39; | GYLQPRTFLLKYNEN | M58;M24;M49;M15;M11; |
| | LTLTRALTA | M14 | GTITDAVDCA | M47 |
| 501 | PGSGVPVVDSYYSLL | M34;M39; | YLQPRTFLLKYNENG | M58;M24;M49;M15;M11; |
| | MPILTLTRAL | M14 | TITDAVDCAL | M47 |
| 502 | GSGVPVVDSYYSLLM | M34;M39; | VGYLQPRTFLLKYNE | M58;M24;M49;M15;M11; |
| | PILTLTRALT | M14 | NGTITDAVDC | M47 |
| 503 | IKIVQMLSDTLKNLSD | M46;M61; | YYVGYLQPRTFLLKY | M58;M24;M49;M15;M11; |
| | RVVFVLWAH | M36 | NENGTITDAV | M47 |
| 504 | RIKIVQMLSDTLKNLS | M46;M61; | LQDLKWARFPKSDGT | M38;M28;M59;M38;M28; |
| | DRVVFVLWA | M36 | GTIYTELEPP | M59 |
| 505 | GQINDMILSLLSKGRLI | M55;M9; | AIGLALYYPSARIVYT | M64;M21;M5;M29;M38; |
| | IRENNRVV | M19 | ACSHAAVDA | M17 |
| 506 | KVFRSSVLHSTQDLFL | M72;M67; | FQEKDEDDNLIDSYF | M71;M54;M18;M15;M52; |
| | PFFSNVTWF | M6 | VVKRHTFSNY | M47 |
| 507 | RSSVLHSTQDLFLPFFS | M72;M67; | RFQEKDEDDNLIDSYF | M71;M54;M18;M15;M52; |
| | NVTWFHAI | M6 | VVKRHTFSN | M47 |
| 508 | VFRSSVLHSTQDLFLPF | M72;M67; | LHVVGPNVNKGEDIQ | M60;M77;M28;M60;M77; |
| | FSNVTWFH | M6 | LLKSAYENFN | M28 |
| 509 | SSVLHSTQDLFLPFFSN | M72;M67; | HCLHVVGPNVNKGE | M60;M77;M28;M60;M77; |
| | VTWFHAIH | M6 | DIQLLKSAYEN | M28 |
| 510 | FRSSVLHSTQDLFLPFF | M72;M67; | CLHVVGPNVNKGEDI | M60;M77;M28;M60;M77; |
| | SNVTWFHA | M6 | QLLKSAYENF | M28 |
| 511 | TLDSKTQSLLIVNNAT | M11;M73; | LEPPCRFVTDTPKGPK | M77;M2;M59;M77;M2;M59 |
| | NVVIKVCEF | M44 | VKYLYFIKG | |
| 512 | GTTLDSKTQSLLIVNN | M11;M73; | YTELEPPCRFVTDTPK | M77;M2;M59;M77;M2;M59 |
| | ATNVVIKVC | M44 | GPKVKYLYF | |
| 513 | DSKTQSLLIVNNATNV | M11;M73; | PPCRFVTDTPKGPKV | M77;M2;M59;M77;M2;M59 |
| | VIKVCEFQF | M44 | KYLYFIKGLN | |
| 514 | TTLDSKTQSLLIVNNA | M11;M73; | ELEPPCRFVTDTPKGP | M77;M2;M59;M77;M2;M59 |
| | TNVVIKVCE | M44 | KVKYLYFIK | |
| 515 | LDSKTQSLLIVNNATN | M11;M73; | EPPCRFVTDTPKGPKV | M77;M2;M59;M77;M2;M59 |
| | VVIKVCEFQ | M44 | KYLYFIKGL | |
| 516 | FKIYSKHTPINLVRDLP | M64;M36; | TELEPPCRFVTDTPKG | M77;M2;M59;M77;M2;M59 |
| | QGFSALEP | M40 | PKVKYLYFI | |
| 517 | YSKHTPINLVRDLPQG | M64;M36; | ECTTIVNGVRRSFYV | M5;M39;M41;M5;M39;M41 |
| | FSALEPLVD | M40 | YANGGKGFCK | |
| 518 | IYSKHTPINLVRDLPQG | M64;M36; | CTTIVNGVRRSFYVY | M5;M39;M41;M5;M39;M41 |
| | FSALEPLV | M40 | ANGGKGFCKL | |
| 519 | SKHTPINLVRDLPQGF | M64;M36; | YTERSEKSYELQTPFE | M25;M34;M39;M25;M34; |
| | SALEPLVDL | M40 | IKLAKKFDT | M39 |
| 520 | GYFKIYSKHTPINLVR | M64;M36; | WYTERSEKSYELQTP | M25;M34;M39;M25;M34; |
| | DLPQGFSAL | M40 | FEIKLAKKFD | M39 |
| 521 | YFKIYSKHTPINLVRDL | M64;M36; | AWYTERSEKSYELQT | M25;M34;M39;M25;M34; |
| | PQGFSALE | M40 | PFEIKLAKKF | M39 |
| 522 | KIYSKHTPINLVRDLPQ | M64;M36; | FYTSKTTVASLINTLN | M57;M72;M68;M57;M72; |
| | GFSALEPL | M40 | DLNETLVTM | M68 |
| 523 | PISFPLCANGQVFGLY | M73;M28; | SKTTVASLINTLNDLN | M57;M72;M68;M57;M72; |
| | KNTCVGSDN | M48 | ETLVTMPLG | M68 |
| 524 | EYVSQPFLMDLEGKQ | M46;M36; | YTSKTTVASLINTLND | M57;M72;M68;M57;M72; |
| | GNFKNLREFV | M20 | LNETLVTMP | M68 |
| 525 | KGVITHDVSSAINRPQI | M36;M21; | TSKTTVASLINTLNDL | M57;M72;M68;M57;M72; |
| | GVVREFLT | M70 | NETLVTMPL | M68 |
| 526 | RPLLESELVIGAVILRG | M55;M53; | KTTVASLINTLNDLNE | M57;M72;M68;M57;M72; |
| | HLRIAGHH | M14 | TLVTMPLGY | M68 |
| 527 | PLLESELVIGAVILRGH | M55;M53; | EHEIAWYTERSEKSY | M25;M30;M49;M25;M30; |
| | LRIAGHHL | M14 | ELQTPFEIKL | M49 |
| 528 | LLESELVIGAVILRGHL | M55;M53; | HEHEIAWYTERSEKS | M25;M30;M49;M25;M30; |
| | RIAGHHLG | M14 | YELQTPFEIK | M49 |
| 529 | FMLWCKDGHVETFYP | M15;M36; | ARFYFYTSKTTVASLI | M29;M72;M68;M29;M72; |
| | KLQSSQAWQP | M47 | NTLNDLNET | M68 |
| 530 | MLWCKDGHVETFYPK | M15;M36; | RFYFYTSKTTVASLIN | M29;M72;M68;M29;M72; |
| | LQSSQAWQPG | M47 | TLNDLNETL | M68 |
| 531 | KDKSAQCFKMFYKGV | M13;M52; | FYFYTSKTTVASLINT | M29;M72;M68;M29;M72; |
| | ITHDVSSAIN | M36 | LNDLNETLV | M68 |
| 532 | ETAHSCNVNRFNVAIT | M33;M25; | IIKTIQPRVEKKKLDG | M57;M26;M29;M57;M26; |
| | RAKVGILCI | M19 | FMGRIRSVY | M29 |
| 533 | SCNVNRFNVAITRAKV | M33;M25; | PQLEQPYVFIKRSDAR | M15;M46;M16;M15;M46; |
| | GILCIMSDR | M19 | TAPHGHVMV | M16 |
| 534 | HSCNVNRFNVAITRAK | M33;M25; | QLEQPYVFIKRSDART | M15;M46;M16;M15;M46; |
| | VGILCIMSD | M19 | APHGHVMVE | M16 |
| 535 | AHSCNVNRFNVAITRA | M33;M25; | LEQPYVFIKRSDARTA | M15;M46;M16;M15;M46; |
| | KVGILCIMS | M19 | PHGHVMVEL | M16 |
| 536 | TAHSCNVNRFNVAITR | M33;M25; | VLPQLEQPYVFIKRSD | M15;M46;M16;M15;M46; |
| | AKVGILCIM | M19 | ARTAPHGHV | M16 |
| 537 | CQEPKLGSLVVRCSFY | M29;M14; | LPQLEQPYVFIKRSDA | M15;M46;M16;M15;M46; |
| | EDFLEYHDV | M70 | RTAPHGHVM | M16 |
| 538 | QEPKLGSLVVRCSFYE | M29;M14; | NKEMYLKLRSDVLLP | M39;M61;M53;M39;M61; |
| | DFLEYHDVR | M70 | LTQYNRYLAL | M53 |
| 539 | LFMRIFTIGTVTLKQGE | M5;M72; | LNKEMYLKLRSDVLL | M43;M39;M61;M43;M39; |
| | IKDATPSD | M6 | PLTQYNRYLA | M61 |
| 540 | FMRIFTIGTVTLKQGEI | M5;M72; | TLVSDIDITFLKKDAP | M57;M11;M58;M57;M11; |
| | KDATPSDF | M6 | YIVGDVVQE | M58 |
| 541 | VATLQAENVTGLFKD | M36;M38; | LVSDIDITFLKKDAPYI | M57;M11;M58;M57;M11; |
| | CSKVITGLHP | M47 | VGDVVQEG | M58 |
| 542 | LQAENVTGLFKDCSK | M36;M38; | VSDIDITFLKKDAPYI | M57;M11;M58;M57;M11; |
| | VITGLHPTQA | M47 | VGDVVQEGV | M58 |
| 543 | ATLQAENVTGLFKDCS | M36;M38; | NYLKSPNFSKLINIIIW | M42;M39;M74;M42;M39; |
| | KVITGLHPT | M47 | FLLLSVCL | M74 |
| 544 | TLQAENVTGLFKDCSK | M36;M38; | YLKSPNFSKLINIIIWF | M42;M39;M74;M42;M39; |
| | VITGLHPTQ | M47 | LLLSVCLG | M74 |
| 545 | AENVTGLFKDCSKVIT | M36;M38; | SFNYLKSPNFSKLINIII | M42;M39;M74;M42;M39; |
| | GLHPTQAPT | M47 | WFLLLSV | M74 |
| 546 | QAENVTGLFKDCSKVI | M36;M38; | FNYLKSPNFSKLINIII | M42;M39;M74;M42;M39; |
| | TGLHPTQAP | M47 | WFLLLSVC | M74 |
| 547 | NVATLQAENVTGLFK | M36;M38; | EVNEFACVVADAVIK | M78;M34;M39;M78;M34; |
| | DCSKVITGLH | M47 | TLQPVSELLT | M39 |
| 548 | TSRTLSYYKLGASQRV | M25;M19; | EEAARYMRSLKVPAT | M5;M72;M35;M5;M72;M35 |
| | AGDSGFAAY | M70 | VSVSSPDAVT | |
| 549 | ATSRTLSYYKLGASQR | M25;M19; | EVEKGVLPQLEQPYV | M15;M16;M74;M15;M16; |
| | VAGDSGFAA | M70 | FIKRSDARTA | M74 |
| 550 | PFTINCQEPKLGSLVV | M29;M82; | KKDWYDFVENPDILR | M64;M21;M32;M49;M43; |
| | RCSFYEDFL | M14 | VYANLGERVR | M22 |
| 551 | TINCQEPKLGSLVVRC | M29;M82; | KIQEGVVDYGARFYF | M29;M67;M41;M29;M67; |
| | SFYEDFLEY | M14 | YTSKTTVASL | M41 |
| 552 | FTINCQEPKLGSLVVR | M29;M82; | IKIQEGVVDYGARFYF | M29;M67;M41;M29;M67; |
| | CSFYEDFLE | M14 | YTSKTTVAS | M41 |
| 553 | NRALTGIAVEQDKNT | M67;M14; | GIKIQEGVVDYGARF | M29;M67;M41;M29;M67; |
| | QEVFAQVKQI | M70 | YFYTSKTTVA | M41 |
| 554 | VYGDFSHSQLGGLHLL | M33;M19; | ELKFNPPALQDAYYR | M75;M39;M74;M75;M39; |
| | IGLAKRFKE | M61 | ARAGEAANFC | M74 |
| 555 | EHVTFFIYNKIVDEPEE | M50;M44; | NGTHWFVTQRNFYEP | M24;M31;M5;M79;M50; |
| | HVQIHTID | M49 | QIITTDNTFV | M18 |
| 556 | VTFFIYNKIVDEPEEHV | M50;M44; | AIDYKHYTPSFKKGA | M82;M27;M16;M82;M27; |
| | QIHTIDGS | M49 | KLLHKPIVWH | M16 |
| 557 | HVTFFIYNKIVDEPEEH | M50;M44; | IDYKHYTPSFKKGAK | M82;M27;M16;M82;M27; |
| | VQIHTIDG | M49 | LLHKPIVWHV | M16 |
| 558 | DWTIEYPIIGDELKINA | M73;M17; | DYKHYTPSFKKGAKL | M82;M27;M16;M82;M27; |
| | ACRKVQHM | M44 | LHKPIVWHVN | M16 |
| 559 | IIGDELKINAACRKVQ | M73;M17; | VAIDYKHYTPSFKKG | M82;M27;M16;M82;M27; |
| | HMVVKAALL | M44 | AKLLHKPIVW | M16 |
| 560 | VDWTIEYPIIGDELKIN | M73;M17; | GVFVSNGTHWFVTQR | M24;M31;M79;M50;M18; |
| | AACRKVQH | M44 | NFYEPQIITT | M35 |
| 561 | IGDELKINAACRKVQH | M73;M17; | VFVSNGTHWFVTQRN | M24;M31;M79;M50;M18; |
| | MVVKAALLA | M44 | FYEPQIITTD | M35 |
| 562 | VTLACFVLAAVYRIN | M33;M58; | IETISLAGSYKDWSYS | M64;M30;M68;M64;M30; |
| | WITGGIAIAM | M44 | GQSTQLGIE | M68 |
| 563 | TTADIVVFDEISMATN | M33;M55; | ETISLAGSYKDWSYS | M64;M30;M68;M64;M30; |
| | YDLSVVNAR | M48 | GQSTQLGIEF | M68 |
| 564 | CWTETDLTKGPHEFCS | M18;M39; | FGGCVFSYVGCHNKC | M40;M75;M55;M40;M75; |
| | QHTMLVKQG | M6 | AYWVPRASAN | M55 |
| 565 | WTETDLTKGPHEFCSQ | M18;M39; | GGCVFSYVGCHNKC | M40;M75;M55;M40;M75; |
| | HTMLVKQGD | M6 | AYWVPRASANI | M55 |
| 566 | VVYRGTTTYKLNVGD | M42;M34; | FSYVGCHNKCAYWV | M40;M75;M55;M40;M75; |
| | YFVLTSHTVM | M44 | PRASANIGCNH | M55 |
| 567 | ACFVLAAVYRINWITG | M50;M58; | GCVFSYVGCHNKCA | M40;M75;M55;M40;M75; |
| | GIAIAMACL | M44 | YWVPRASANIG | M55 |
| 568 | DKVAGFAKFLKTNCC | M55;M73; | CVFSYVGCHNKCAY | M40;M75;M55;M40;M75; |
| | RFQEKDEDDN | M8 | WVPRASANIGC | M55 |
| 569 | DETQALPQRQKKQQT | M13;M29; | SYVGCHNKCAYWVP | M40;M75;M55;M40;M75; |
| | VTLLPAADLD | M14 | RASANIGCNHT | M55 |
| 570 | IGEYTFEKGDYGDAV | M39;M9; | VFSYVGCHNKCAYW | M40;M75;M55;M40;M75; |
| | VYRGTTTYKL | M12 | VPRASANIGCN | M55 |
| 571 | QIGEYTFEKGDYGDA | M39;M9; | GICASYQTQTNSPRRA | M31;M70;M5;M25;M50; |
| | VVYRGTTTYK | M12 | RSVASQSII | M18 |
| 572 | TVVIGTSKFYGGWHN | M18;M72; | ICASYQTQTNSPRRAR | M31;M70;M5;M25;M50; |
| | MLKTVYSDVE | M6 | SVASQSIIA | M18 |
| 573 | FPPTSFGPLVRKIFVDG | M71;M1; | LVRGFGDSVEEVLSE | M55;M10;M70;M55;M10; |
| | VPFVVSTG | M54 | ARQHLKDGTC | M70 |
| 574 | VLFSTVFPPTSFGPLVR | M71;M1; | VRGFGDSVEEVLSEA | M55;M10;M70;M55;M10; |
| | KIFVDGVP | M54 | RQHLKDGTCG | M70 |
| 575 | VFPPTSFGPLVRKIFVD | M71;M1; | ATRVECTTIVNGVRR | M5;M29;M41;M5;M29;M41 |
| | GVPFVVST | M54 | SFYVYANGGK | |
| 576 | FSTVFPPTSFGPLVRKI | M71;M1; | TRVECTTIVNGVRRSF | M5;M29;M41;M5;M29;M41 |
| | FVDGVPFV | M54 | YVYANGGKG | |
| 577 | TVFPPTSFGPLVRKIFV | M71;M1; | VECTTIVNGVRRSFY | M5;M29;M41;M5;M29;M41 |
| | DGVPFVVS | M54 | VYANGGKGFC | |
| 578 | LFSTVFPPTSFGPLVRK | M71;M1; | RVECTTIVNGVRRSFY | M5;M29;M41;M5;M29;M41 |
| | IFVDGVPF | M54 | VYANGGKGF | |
| 579 | STVFPPTSFGPLVRKIF | M71;M1; | RATRVECTTIVNGVR | M5;M29;M41;M5;M29;M41 |
| | VDGVPFVV | M54 | RSFYVYANGG | |
| 580 | STDTGVEHVTFFIYNKI | M50;M47; | NRYLALYNKYKYFSG | M67;M66;M31;M67;M66; |
| | VDEPEEHV | M44 | AMDTTSYREA | M31 |
| 581 | LSTDTGVEHVTFFIYN | M50;M47; | RYLALYNKYKYFSGA | M67;M66;M31;M67;M66; |
| | KIVDEPEEH | M44 | MDTTSYREAA | M31 |
| 582 | VECFDKFKVNSTLEQY | M50;M18; | FRRAFGEYSHVVAFN | M55;M60;M56;M55;M60; |
| | VFCTVNALP | M44 | TLLFLMSFTV | M56 |
| 583 | PARARVECFDKFKVNS | M50;M18; | RAFGEYSHVVAFNTL | M55;M60;M56;M55;M60; |
| | TLEQYVFCT | M44 | LFLMSFTVLC | M56 |
| 584 | ARVECFDKFKVNSTLE | M50;M18; | AFGEYSHVVAFNTLL | M55;M60;M56;M55;M60; |
| | QYVFCTVNA | M44 | FLMSFTVLCL | M56 |
| 585 | RVECFDKFKVNSTLEQ | M50;M18; | RRAFGEYSHVVAFNT | M55;M60;M56;M55;M60; |
| | YVFCTVNAL | M44 | LLFLMSFTVL | M56 |
| 586 | ARARVECFDKFKVNS | M50;M18; | RFRRAFGEYSHVVAF | M55;M60;M56;M55;M60; |
| | TLEQYVFCTV | M44 | NTLLFLMSFT | M56 |
| 587 | RARVECFDKFKVNSTL | M50;M18; | TILDGISQYSLRLIDA | M57;M60;M54;M57;M60; |
| | EQYVFCTVN | M44 | MMFTSDLAT | M54 |
| 588 | AWNSNNLDSKVGGNY | M1;M16; | ILDGISQYSLRLIDAM | M57;M60;M54;M57;M60; |
| | NYLYRLFRKS | M37 | MFTSDLATN | M54 |
| 589 | SQLGGLHLLIGLAKRF | M19;M61; | SRIKASMPTTIAKNTV | M11;M9;M24;M11;M9;M24 |
| | KESPFELED | M20 | KSVGKFCLE | |
| 590 | LGGLHLLIGLAKRFKE | M19;M61; | SVLQQLRVESSSKLW | M11;M34;M82;M11;M34; |
| | SPFELEDFI | M20 | AQCVQLHNDI | M82 |
| 591 | GGLHLLIGLAKRFKES | M19;M61; | SEFDRDAAMQRKLEK | M57;M82;M50;M57;M82; |
| | PFELEDFIP | M20 | MADQAMTQMY | M50 |
| 592 | QLGGLHLLIGLAKRFK | M19;M61; | KSEFDRDAAMQRKLE | M57;M82;M50;M57;M82; |
| | ESPFELEDF | M20 | KMADQAMTQM | M50 |
| 593 | RIGMEVTPSGTWLTYT | M41;M19; | AKSEFDRDAAMQRK | M57;M82;M50;M57;M82; |
| | GAIKLDDKD | M23 | LEKMADQAMTQ | M50 |
| 594 | IAATRGATVVIGTSKF | M72;M2; | VAKSEFDRDAAMQR | M57;M82;M50;M57;M82; |
| | YGGWHNMLK | M17 | KLEKMADQAMT | M50 |
| 595 | KSIAATRGATVVIGTS | M72;M2; | RQCSGVTFQSAVKRTI | M33;M9;M71;M33;M9;M71 |
| | KFYGGWHNM | M17 | KGTHHWLLL | |
| 596 | SIAATRGATVVIGTSKF | M72;M2; | HINAQVAKSHNIALI | M32;M72;M68;M32;M72; |
| | YGGWHNML | M17 | WNVKDFMSLS | M68 |
| 597 | LPFGWLIVGVALLAVF | M14;M26; | RHINAQVAKSHNIALI | M32;M72;M68;M32;M72; |
| | QSASKIITL | M61 | WNVKDFMSL | M68 |
| 598 | SLPFGWLIVGVALLAV | M14;M26; | MTARTVYDDGARRV | M5;M11;M20;M5;M11;M20 |
| | FQSASKIIT | M61 | WTLMNVLTLVY | |
| 599 | DLLKYDFTEERLKLFD | M55;M7; | GDCLGDIAARDLICA | M58;M78;M65;M10;M79; |
| | RYFKYWDQT | M53 | QKFNGLTVLP | M50 |
| 600 | CRLMKTIGPDMFLGTC | M6;M16; | MMSAPPAQYELKHG | M33;M65;M66;M33;M65; |
| | RRCPAEIVD | M37 | TFTCASEYTGN | M66 |
| 601 | INDMILSLLSKGRLIIRE | M55;M9; | PAQYELKHGTFTCAS | M33;M65;M66;M33;M65; |
| | NNRVVIS | M48 | EYTGNYQCGH | M66 |
| 602 | NDMILSLLSKGRLIIRE | M55;M9; | MSAPPAQYELKHGTF | M33;M65;M66;M33;M65; |
| | NNRVVISS | M48 | TCASEYTGNY | M66 |
| 603 | MIERFVSLAIDAYPLT | M55;M5; | APPAQYELKHGTFTC | M33;M65;M66;M33;M65; |
| | KHPNQEYAD | M2 | ASEYTGNYQC | M66 |
| 604 | SIGFDYVYNPFMIDVQ | M57;M1; | SAPPAQYELKHGTFT | M33;M65;M66;M33;M65; |
| | QWGFTGNLQ | M20 | CASEYTGNYQ | M66 |
| 605 | IGFDYVYNPFMIDVQQ | M57;M1; | AQYELKHGTFTCASE | M33;M65;M66;M33;M65; |
| | WGFTGNLQS | M20 | YTGNYQCGHY | M66 |
| 606 | RVYSSANNCTFEYVSQ | M57;M1; | PPAQYELKHGTFTCA | M33;M65;M66;M33;M65; |
| | PFLMDLEGK | M20 | SEYTGNYQCG | M66 |
| 607 | GFDYVYNPFMIDVQQ | M57;M1; | LARKHTTCCSLSHRF | M81;M5;M60;M79;M43; |
| | WGFTGNLQSN | M20 | YRLANECAQV | M42 |
| 608 | PSASAFFGMSRIGMEV | M5;M28; | QPITNCVKMLCTHTG | M64;M22;M58;M64;M22; |
| | TPSGTWLTY | M16 | TGQAITVTPE | M58 |
| 609 | KFALTCFSTQFAFACP | M33;M22; | PITNCVKMLCTHTGT | M64;M22;M58;M64;M22; |
| | DGVKHVYQL | M6 | GQAITVTPEA | M58 |
| 610 | FALTCFSTQFAFACPD | M33;M22; | ITNCVKMLCTHTGTG | M64;M22;M58;M64;M22; |
| | GVKHVYQLR | M6 | QAITVTPEAN | M58 |
| 611 | NKFALTCFSTQFAFAC | M33;M22; | NWNTKHSSGVTREL | M60;M22;M74;M60;M22; |
| | PDGVKHVYQ | M6 | MRELNGGAYTR | M74 |
| 612 | IGDPAQLPAPRTLLTK | M29;M6; | VTTTLEETKFLTENLL | M50;M11;M20;M50;M11; |
| | GTLEPEYFN | M58 | LYIDINGNL | M20 |
| 613 | GDPAQLPAPRTLLTKG | M29;M6; | EVTTTLEETKFLTENL | M50;M11;M20;M50;M11; |
| | TLEPEYFNS | M58 | LLYIDINGN | M20 |
| 614 | YIGDPAQLPAPRTLLT | M29;M6; | EEKFKEGVEFLRDGW | M57;M27;M20;M57;M27; |
| | KGTLEPEYF | M58 | EIVKFISTCA | M20 |
| 615 | DPAQLPAPRTLLTKGT | M29;M6; | LEEKFKEGVEFLRDG | M57;M27;M20;M57;M27; |
| | LEPEYFNSV | M58 | WEIVKFISTC | M20 |
| 616 | WESGVKDCVVLHSYF | M46;M14; | WLEEKFKEGVEFLRD | M57;M27;M20;M57;M27; |
| | TSDYYQLYST | M20 | GWEIVKFIST | M20 |
| 617 | EKWESGVKDCVVLHS | M46;M14; | FLQSINFVRIIMRLWL | M75;M24;M10;M76;M9; |
| | YFTSDYYQLY | M20 | CWKCRSKNP | M23 |
| 618 | KWESGVKDCVVLHSY | M46;M14; | LQSINFVRIIMRLWLC | M75;M24;M10;M76;M9; |
| | FTSDYYQLYS | M20 | WKCRSKNPL | M23 |
| 619 | SVFNICQAVTANVNAL | M11;M9; | GVTRDIASTDTCFAN | M64;M22;M47;M64;M22; |
| | LSTDGNKIA | M8 | KHADFDTWFS | M47 |
| 620 | PQIAQFAPSASAFFGM | M28;M16; | VTRDIASTDTCFANK | M64;M22;M47;M64;M22; |
| | SRIGMEVTP | M70 | HADFDTWFSQ | M47 |
| 621 | WPQIAQFAPSASAFFG | M28;M16; | HWFVTQRNFYEPQIIT | M64;M10;M5;M79;M9;M23 |
| | MSRIGMEVT | M70 | TDNTFVSGN | |
| 622 | NKTTLPVNVAFELWA | M55;M28; | EDEFTPFDVVRQCSG | M46;M60;M54;M46;M60; |
| | KRNIKPVPEV | M74 | VTFQSAVKRT | M54 |
| 623 | ENKTTLPVNVAFELW | M55;M28; | AIDGGVTRDIASTDTC | M64;M9;M22;M64;M9;M22 |
| | AKRNIKPVPE | M74 | FANKHADFD | |
| 624 | HFRELGVVHNQDVNL | M53;M38; | YKAIDGGVTRDIAST | M64;M9;M22;M64;M9;M22 |
| | HSSRLSFKEL | M14 | DTCFANKHAD | |
| 625 | NTNSSPDDQIGYYRRA | M55;M16; | GYKAIDGGVTRDIAS | M64;M9;M22;M64;M9;M22 |
| | TRRIRGGDG | M71 | TDTCFANKHA | |
| 626 | NSSPDDQIGYYRRATR | M55;M16; | IDGGVTRDIASTDTCF | M64;M9;M22;M64;M9;M22 |
| | RIRGGDGKM | M71 | ANKHADFDT | |
| 627 | TNSSPDDQIGYYRRAT | M55;M16; | KAIDGGVTRDIASTDT | M64;M9;M22;M64;M9;M22 |
| | RRIRGGDGK | M71 | CFANKHADF | |
| 628 | SSPDDQIGYYRRATRR | M55;M16; | DGGVTRDIASTDTCF | M64;M9;M22;M64;M9;M22 |
| | IRGGDGKMK | M71 | ANKHADFDTW | |
| 629 | DKRTTCFSVAALTNN | M1;M22; | VYFLQSINFVRIIMRL | M75;M24;M10;M25;M9; |
| | VAFQTVKPGN | M73 | WLCWKCRSK | M23 |
| 630 | WKFYDAQPCSDKAYK | M29;M6; | MTWLDMVDTSLSGF | M64;M75;M22;M64;M75; |
| | IEELFYSYAT | M59 | KLKDCVMYASA | M22 |
| 631 | FYDAQPCSDKAYKIEE | M29;M6; | IQQVVDADSKIVQLSE | M60;M54;M49;M60;M54; |
| | LFYSYATHS | M59 | ISMDNSPNL | M49 |
| 632 | YDAQPCSDKAYKIEEL | M29;M6; | EIQQVVDADSKIVQLS | M60;M54;M49;M60;M54; |
| | FYSYATHSD | M59 | EISMDNSPN | M49 |
| 633 | KFYDAQPCSDKAYKIE | M29;M6; | TAVVIPTKKAGGTTE | M78;M58;M17;M78;M58; |
| | ELFYSYATH | M59 | MLAKALRKVP | M17 |
| 634 | EWKFYDAQPCSDKAY | M29;M6; | VETKAIVSTIQRKYKG | M40;M5;M60;M40;M5;M60 |
| | KIEELFYSYA | M59 | IKIQEGVVD | |
| 635 | YTKRNVIPTITQMNLK | M14;M58; | ETKAIVSTIQRKYKGI | M40;M5;M60;M40;M5;M60 |
| | YAISAKNRA | M59 | KIQEGVVDY | |
| 636 | QTGKIADYNYKLPDDF | M13;M67; | EQKIAEIPKEEVKPFIT | M10;M26;M24;M10;M26; |
| | TGCVIAWNS | M12 | ESKPSVEQ | M24 |
| 637 | GQTGKIADYNYKLPD | M13;M67; | TFQSAVKRTIKGTHH | M52;M9;M24;M52;M9;M24 |
| | DFTGCVIAWN | M12 | WLLLTILTSL | |
| 638 | GVYFASTEKSNIIRGWI | M6;M24; | QSAVKRTIKGTHHWL | M52;M9;M24;M52;M9;M24 |
| | FGTTLDSK | M70 | LLTILTSLLV | |
| 639 | NLKQLPFFYYSDSPCE | M32;M41; | FQSAVKRTIKGTHHW | M52;M9;M24;M52;M9;M24 |
| | SHGKQVVSD | M45 | LLLTILTSLL | |
| 640 | LKQLPFFYYSDSPCES | M32;M41; | VTFQSAVKRTIKGTH | M52;M9;M24;M52;M9;M24 |
| | HGKQVVSDI | M45 | HWLLLTILTS | |
| 641 | FVNLKQLPFFYYSDSP | M32;M41; | GSALLEDEFTPFDVVR | M46;M60;M41;M46;M60; |
| | CESHGKQVV | M45 | QCSGVTFQS | M41 |
| 642 | VNLKQLPFFYYSDSPC | M32;M41; | MDTTSYREAACCHLA | M78;M5;M79;M78;M5;M79 |
| | ESHGKQVVS | M45 | KALNDFSNSG | |
| 643 | AFVNLKQLPFFYYSDS | M32;M41; | AMDTTSYREAACCHL | M78;M5;M79;M78;M5;M79 |
| | PCESHGKQV | M45 | AKALNDFSNS | |
| 644 | LRKHFSMMILSDDAV | M57;M13; | HGGGVAGALNKATN | M78;M79;M33;M78;M79; |
| | VCFNSTYASQ | M7 | NAMQVESDDYI | M33 |
| 645 | YAYLRKHFSMMILSD | M57;M13; | GGGVAGALNKATNN | M78;M79;M33;M78;M79; |
| | DAVVCFNSTY | M7 | AMQVESDDYIA | M33 |
| 646 | YLRKHFSMMILSDDA | M57;M13; | YLKHGGGVAGALNK | M78;M79;M33;M78;M79; |
| | VVCFNSTYAS | M7 | ATNNAMQVESD | M33 |
| 647 | FYAYLRKHFSMMILSD | M57;M13; | GVAGALNKATNNAM | M78;M79;M33;M78;M79; |
| | DAVVCFNST | M7 | QVESDDYIATN | M33 |
| 648 | AYLRKHFSMMILSDD | M57;M13; | VAGALNKATNNAMQ | M78;M79;M33;M78;M79; |
| | AVVCFNSTYA | M7 | VESDDYIATNG | M33 |
| 649 | LSDRELHLSWEVGKPR | M5;M12; | LKHGGGVAGALNKA | M78;M79;M33;M78;M79; |
| | PPLNRNYVF | M70 | TNNAMQVESDD | M33 |
| 650 | SDRELHLSWEVGKPRP | M5;M12; | KHGGGVAGALNKAT | M78;M79;M33;M78;M79; |
| | PLNRNYVFT | M70 | NNAMQVESDDY | M33 |
| 651 | DALFAYTKRNVIPTIT | M14;M59; | GGVAGALNKATNNA | M78;M79;M33;M78;M79; |
| | QMNLKYAIS | M10 | MQVESDDYIAT | M33 |
| 652 | LQSLQTYVTQQLIRAA | M21;M12; | HTKKWKYPQVNGLT | M60;M54;M74;M60;M54; |
| | EIRASANLA | M49 | SIKWADNNCYL | M74 |
| 653 | GRLQSLQTYVTQQLIR | M21;M12; | KSEKQVEQKIAEIPKE | M10;M24;M74;M10;M24; |
| | AAEIRASAN | M49 | EVKPFITES | M74 |
| 654 | RLQSLQTYVTQQLIRA | M21;M12; | MKSEKQVEQKIAEIPK | M10;M24;M74;M10;M24; |
| | AEIRASANL | M49 | EEVKPFITE | M74 |
| 655 | LPQGTTLPKGFYAEGS | M75;M42; | EMKSEKQVEQKIAEIP | M10;M24;M74;M10;M24; |
| | RGGSQASSR | M45 | KEEVKPFIT | M74 |
| 656 | QLPQGTTLPKGFYAEG | M75;M42; | SEKQVEQKIAEIPKEE | M10;M24;M74;M10;M24; |
| | SRGGSQASS | M45 | VKPFITESK | M74 |
| 657 | PQGTTLPKGFYAEGSR | M75;M42; | EKQVEQKIAEIPKEEV | M10;M24;M74;M10;M24; |
| | GGSQASSRS | M45 | KPFITESKP | M74 |
| 658 | QGTTLPKGFYAEGSRG | M75;M42; | SFLEMKSEKQVEQKI | M10;M24;M74;M10;M24; |
| | GSQASSRSS | M45 | AEIPKEEVKP | M74 |
| 659 | PFLGVYYHKNNKSWM | M22;M28; | FLEMKSEKQVEQKIA | M10;M24;M74;M10;M24; |
| | ESEFRVYSSA | M70 | EIPKEEVKPF | M74 |
| 660 | DGYVMHANYIFWRNT | M41;M9; | LEMKSEKQVEQKIAEI | M10;M24;M74;M10;M24; |
| | NPIQLSSYSL | M8 | PKEEVKPFI | M74 |
| 661 | MHANYIFWRNTNPIQL | M41;M9; | IGGAKLKALNLGETF | M18;M27;M24;M18;M27; |
| | SSYSLFDMS | M8 | VTHSKGLYRK | M24 |
| 662 | GYVMHANYIFWRNTN | M41;M9; | DSAEVAVKMFDAYV | M26;M41;M37;M26;M41; |
| | PIQLSSYSLF | M8 | NTFSSTFNVPM | M37 |
| 663 | VMHANYIFWRNTNPI | M41;M9; | VGDSAEVAVKMFDA | M26;M41;M37;M26;M41; |
| | QLSSYSLFDM | M8 | YVNTFSSTFNV | M37 |
| 664 | HANYIFWRNTNPIQLS | M41;M9; | GDSAEVAVKMFDAY | M26;M41;M37;M26;M41; |
| | SYSLFDMSK | M8 | VNTFSSTFNVP | M37 |
| 665 | YVMHANYIFWRNTNP | M41;M9; | KGLYRKCVKSREETG | M60;M54;M38;M60;M54; |
| | IQLSSYSLFD | M8 | LLMPLKAPKE | M38 |
| 666 | AGLEAPFLYLYALVYF | M60;M54; | PSYAAFATAQEAYEQ | M33;M68;M31;M33;M68; |
| | LQSINFVRI | M12 | AVANGDSEVV | M31 |
| 667 | AAGLEAPFLYLYALV | M60;M54; | SKGLYRKCVKSREET | M52;M38;M47;M52;M38; |
| | YFLQSINFVR | M12 | GLLMPLKAPK | M47 |
| 668 | KLFDRYFKYWDQTYH | M50;M18; | LGPLSAQTGIAVLDM | M32;M24;M59;M32;M24; |
| | PNCVNCLDDR | M8 | CASLKELLQN | M59 |
| 669 | FDRYFKYWDQTYHPN | M50;M18; | KKWKYPQVNGLTSIK | M60;M54;M23;M60;M54; |
| | CVNCLDDRCI | M8 | WADNNCYLAT | M23 |
| 670 | WDQTYHPNCVNCLDD | M50;M18; | TKKWKYPQVNGLTSI | M60;M54;M23;M60;M54; |
| | RCILHCANFN | M8 | KWADNNCYLA | M23 |
| 671 | KYWDQTYHPNCVNCL | M50;M18; | KWKYPQVNGLTSIK | M60;M54;M23;M60;M54; |
| | DDRCILHCAN | M8 | WADNNCYLATA | M23 |
| 672 | RYFKYWDQTYHPNCV | M50;M18; | YPQVNGLTSIKWADN | M60;M54;M23;M60;M54; |
| | NCLDDRCILH | M8 | NCYLATALLT | M23 |
| 673 | YWDQTYHPNCVNCLD | M50;M18; | WKYPQVNGLTSIKW | M60;M54;M23;M60;M54; |
| | DRCILHCANF | M8 | ADNNCYLATAL | M23 |
| 674 | LFDRYFKYWDQTYHP | M50;M18; | KYPQVNGLTSIKWAD | M60;M54;M23;M60;M54; |
| | NCVNCLDDRC | M8 | NNCYLATALL | M23 |
| 675 | FKYWDQTYHPNCVNC | M50;M18; | VCINGLMLLEIKDTEK | M78;M9;M61;M78;M9;M61 |
| | LDDRCILHCA | M8 | YCALAPNMM | |
| 676 | YFKYWDQTYHPNCVN | M50;M18; | PVCINGLMLLEIKDTE | M78;M9;M61;M78;M9;M61 |
| | CLDDRCILHC | M8 | KYCALAPNM | |
| 677 | DRYFKYWDQTYHPNC | M50;M18; | CINGLMLLEIKDTEKY | M78;M9;M61;M78;M9;M61 |
| | VNCLDDRCIL | M8 | CALAPNMMV | |
| 678 | TVKPGNFNKDFYDFA | M33;M63; | NEKQEILGTVSWNLR | M75;M9;M24;M75;M9;M24 |
| | VSKGFFKEGS | M70 | EMLAHAEETR | |
| 679 | AFQTVKPGNFNKDFY | M33;M63; | SNEKQEILGTVSWNL | M75;M9;M24;M75;M9;M24 |
| | DFAVSKGFFK | M70 | REMLAHAEET | |
| 680 | VKPGNFNKDFYDFAV | M33;M63; | YLKLRSDVLLPLTQY | M69;M53;M61;M69;M53; |
| | SKGFFKEGSS | M70 | NRYLALYNKY | M61 |
| 681 | FQTVKPGNFNKDFYDF | M33;M63; | EMYLKLRSDVLLPLT | M69;M53;M61;M69;M53; |
| | AVSKGFFKE | M70 | QYNRYLALYN | M61 |
| 682 | QTVKPGNFNKDFYDF | M33;M63; | MYLKLRSDVLLPLTQ | M69;M53;M61;M69;M53; |
| | AVSKGFFKEG | M70 | YNRYLALYNK | M61 |
| 683 | AISAKNRARTVAGVSI | M60;M22; | AAFATAQEAYEQAV | M33;M47;M59;M33;M47; |
| | CSTMTNRQF | M2 | ANGDSEVVLKK | M59 |
| 684 | EFRVYSSANNCTFEYV | M1;M82; | AFATAQEAYEQAVA | M33;M47;M59;M33;M47; |
| | SQPFLMDLE | M20 | NGDSEVVLKKL | M59 |
| 685 | NLIIKNLSKSLTENKYS | M11;M73; | YAAFATAQEAYEQA | M33;M47;M59;M33;M47; |
| | QLDEEQPM | M28 | VANGDSEVVLK | M59 |
| 686 | DLLFNKVTLADAGFIK | M41;M48; | GARRVWTLMNVLTL | M37;M24;M20;M37;M24; |
| | QYGDCLGDI | M70 | VYKVYYGNALD | M20 |
| 687 | KQGNFKNLREFVFKNI | M6;M54; | DTKRGVYCCREHEHE | M75;M27;M74;M75;M27; |
| | DGYFKIYSK | M59 | IAWYTERSEK | M74 |
| 688 | PFLMDLEGKQGNFKN | M6;M54; | ESGEFKLASHMYCSF | M23;M52;M20;M23;M52; |
| | LREFVFKNID | M59 | YPPDEDEEEG | M20 |
| 689 | QPFLMDLEGKQGNFK | M6;M54; | DESGEFKLASHMYCS | M23;M52;M20;M23;M52; |
| | NLREFVFKNI | M59 | FYPPDEDEEE | M20 |
| 690 | GKQGNFKNLREFVFK | M6;M54; | TAQEAYEQAVANGD | M33;M13;M59;M33;M13; |
| | NIDGYFKIYS | M59 | SEVVLKKLKKS | M59 |
| 691 | TNSPRRARSVASQSIIA | M5;M6; | SSSKLWAQCVQLHND | M32;M82;M68;M32;M82; |
| | YTMSLGAE | M59 | ILLAKDTTEA | M68 |
| 692 | PYNSVTSSIVITSGDGT | M29;M28; | IICISTKHFYWFFSNYL | M50;M18;M23;M50;M18; |
| | TSPISEHD | M62 | KRRVVFNG | M23 |
| 693 | ECLYRNRDVDTDFVN | M26;M6; | TCANDPVGFTLKNTV | M15;M75;M10;M15;M75; |
| | EFYAYLRKHF | M59 | CTVCGMWKGY | M10 |
| 694 | YECLYRNRDVDTDFV | M26;M6; | IPTTCANDPVGFTLKN | M15;M75;M10;M15;M75; |
| | NEFYAYLRKH | M59 | TVCTVCGMW | M10 |
| 695 | PTWRVYSTGSNVFQT | M75;M82; | TTCANDPVGFTLKNT | M15;M75;M10;M15;M75; |
| | RAGCLIGAEH | M8 | VCTVCGMWKG | M10 |
| 696 | IAWNSNNLDSKVGGN | M50;M1; | PTTCANDPVGFTLKN | M15;M75;M10;M15;M75; |
| | YNYLYRLFRK | M37 | TVCTVCGMWK | M10 |
| 697 | TSTDVVYRAFDIYNDK | M79;M7; | QIPTTCANDPVGFTLK | M15;M75;M10;M15;M75; |
| | VAGFAKFLK | M41 | NTVCTVCGM | M10 |
| 698 | MLRIMASLVLARKHT | M5;M2; | VQIPTTCANDPVGFTL | M15;M75;M10;M15;M75; |
| | TCCSLSHRFY | M35 | KNTVCTVCG | M10 |
| 699 | NMLRIMASLVLARKH | M5;M2; | FIETISLAGSYKDWSY | M64;M32;M68;M64;M32; |
| | TTCCSLSHRF | M35 | SGQSTQLGI | M68 |
| 700 | EASKKPRQKRTATKA | M5;M2; | LPTEVLTEEVVLKTG | M60;M38;M62;M60;M38; |
| | YNVTQAFGRR | M70 | DLQPLEQPTS | M62 |
| 701 | AEASKKPRQKRTATK | M5;M2; | GLDYKAFKQIVESCG | M23;M42;M37;M23;M42; |
| | AYNVTQAFGR | M70 | NFKVTKGKAK | M37 |
| 702 | PIDKCSRIIPARARVEC | M33;M26; | LDYKAFKQIVESCGN | M23;M42;M37;M23;M42; |
| | FDKFKVNS | M28 | FKVTKGKAKK | M37 |
| 703 | EPEFYEAMYTPHTVLQ | M75;M39; | PFLNKVVSTTTNIVTR | M78;M23;M37;M78;M23; |
| | AVGACVLCN | M16 | CLNRVCTNY | M37 |
| 704 | NDNTSRYWEPEFYEA | M75;M39; | KCAYWVPRASANIGC | M75;M9;M38;M75;M9;M38 |
| | MYTPHTVLQA | M16 | NHTGVVGEGS | |
| 705 | TNDNTSRYWEPEFYE | M75;M39; | CAYWVPRASANIGCN | M75;M9;M38;M75;M9;M38 |
| | AMYTPHTVLQ | M16 | HTGVVGEGSE | |
| 706 | WEPEFYEAMYTPHTV | M75;M39; | SGSDVLYQPPQTSITS | M38;M81;M62;M38;M81; |
| | LQAVGACVLC | M16 | AVLQSGFRK | M62 |
| 707 | CQAVTANVNALLSTD | M10;M9; | GSDVLYQPPQTSITSA | M38;M81;M62;M38;M81; |
| | GNKIADKYVR | M8 | VLQSGFRKM | M62 |
| 708 | FWNCNVDRYPANSIV | M50;M18; | ALEPLVDLPIGINITRF | M4;M5;M1;M3;M2;M22; |
| | CRFDTRVLSN | M48 | QTLLALHR | M20 |
| 709 | WNCNVDRYPANSIVC | M50;M18; | LEPLVDLPIGINITRFQ | M4;M5;M1;M3;M2;M22; |
| | RFDTRVLSNL | M48 | TLLALHRS | 0M2 |
| 710 | LLLLFVTVYSHLLLVA | M43;M6; | NNCTFEYVSQPFLMD | M4;M1;M3;M57;M36;M46; |
| | AGLEAPFLY | M37 | LEGKQGNFKN | M20 |
| 711 | LLLFVTVYSHLLLVAA | M43;M6; | LPAPRTLLTKGTLEPE | M4;M58;M16;M1;M29;M3; |
| | GLEAPFLYL | M37 | YFNSVCRLM | M6 |
| 712 | DNKLKAHKDKSAQCF | M13;M63; | RTLLTKGTLEPEYFNS | M4;M13;M16;M1;M3;M6; |
| | KMFYKGVITH | M52 | VCRLMKTIG | M15 |
| 713 | NKLKAHKDKSAQCFK | M13;M63; | PAPRTLLTKGTLEPEY | M4;M13;M58;M16;M1;M3; |
| | MFYKGVITHD | M52 | FNSVCRLMK | M6 |
| 714 | ELTSMKYFVKIGPERT | M27;M28; | APRTLLTKGTLEPEYF | M4;M13;M58;M16;M1;M3; |
| | CCLCDRRAT | M66 | NSVCRLMKT | M6 |
| 715 | AHGFELTSMKYFVKIG | M27;M28; | CLDDRCILHCANFNV | M4;M64;M24;M1;M3;M48; |
| | PERTCCLCD | M66 | LFSTVFPPTS | M23 |
| 716 | VLWAHGFELTSMKYF | M27;M28; | NCVNCLDDRCILHCA | M4;M64;M24;M1;M3;M48; |
| | VKIGPERTCC | M66 | NFNVLFSTVF | M23 |
| 717 | TSMKYFVKIGPERTCC | M27;M28; | CVNCLDDRCILHCAN | M4;M64;M24;M1;M3;M48; |
| | LCDRRATCF | M66 | FNVLFSTVFP | M23 |
| 718 | HGFELTSMKYFVKIGP | M27;M28; | NCLDDRCILHCANFN | M4;M64;M24;M1;M3;M48; |
| | ERTCCLCDR | M66 | VLFSTVFPPT | M23 |
| 719 | LWAHGFELTSMKYFV | M27;M28; | VNCLDDRCILHCANF | M4;M64;M24;M1;M3;M48; |
| | KIGPERTCCL | M66 | NVLFSTVFPP | M23 |
| 720 | GFELTSMKYFVKIGPE | M27;M28; | FLYLYALVYFLQSINF | M4;M12;M54;M19;M60; |
| | RTCCLCDRR | M66 | VRIIMRLWL | M25;M3 |
| 721 | FVLWAHGFELTSMKY | M27;M28; | PFLYLYALVYFLQSIN | M4;M12;M54;M19;M60; |
| | FVKIGPERTC | M66 | FVRIIMRLW | M25;M3 |
| 722 | FELTSMKYFVKIGPER | M27;M28; | NRDVDTDFVNEFYAY | M4;M71;M16;M3;M6;M52; |
| | TCCLCDRRA | M66 | LRKHFSMMIL | M26 |
| 723 | WAHGFELTSMKYFVK | M27;M28; | LYLYALVYFLQSINFV | M4;M54;M19;M60;M25;M3; |
| | IGPERTCCLC | M66 | RIIMRLWLC | M9 |
| 724 | VFVLWAHGFELTSMK | M27;M28; | AGCFVDDIVKTDGTL | M9;M10;M7;M3;M6;M8; |
| | YFVKIGPERT | M66 | MIERFVSLAI | M11 |
| 725 | LTSMKYFVKIGPERTC | M27;M28; | GAGCFVDDIVKTDGT | M9;M10;M7;M3;M6;M8; |
| | CLCDRRATC | M66 | LMIERFVSLA | M11 |
| 726 | LQHRLYECLYRNRDV | M47;M6; | LKSFTVEKGIYQTSNF | M1;M25;M3;M37;M41;M8; |
| | DTDFVNEFYA | M59 | RVQPTESIV | M23 |
| 727 | SSQCVNLTTRTQLPPA | M9;M2; | TLKSFTVEKGIYQTSN | M1;M25;M3;M37;M41;M8; |
| | YTNSFTRGV | M24 | FRVQPTESI | M23 |
| 728 | LVSSQCVNLTTRTQLP | M9;M2; | AEIRASANLAATKMS | M4;M64;M40;M3;M48;M27; |
| | PAYTNSFTR | M24 | ECVLGQSKRV | M23 |
| 729 | VSSQCVNLTTRTQLPP | M9;M2; | AAEIRASANLAATKM | M4;M64;M40;M3;M48;M27; |
| | AYTNSFTRG | M24 | SECVLGQSKR | M23 |
| 730 | VAFQTVKPGNFNKDF | M63;M59; | YGGWHNMLKTVYSD | M32;M6;M45;M3;M18;M43; |
| | YDFAVSKGFF | M70 | VENPHLMGWDY | M59 |
| 731 | LGVYYHKNNKSWME | M41;M28; | KFYGGWHNMLKTVY | M32;M6;M45;M3;M18;M43; |
| | SEFRVYSSANN | M70 | SDVENPHLMGW | M59 |
| 732 | SSQAWQPGVAMPNLY | M9;M12; | FYGGWHNMLKTVYS | M32;M6;M45;M3;M18;M43; |
| | KMQRMLLEKC | M23 | DVENPHLMGWD | M59 |
| 733 | YVFCTVNALPETTADI | M38;M28; | GGWHNMLKTVYSDV | M32;M6;M45;M3;M18;M43; |
| | VVFDEISMA | M49 | ENPHLMGWDYP | M59 |
| 734 | MHAASGNLLLDKRTT | M50;M7; | SKFYGGWHNMLKTV | M32;M6;M45;M3;M18;M43; |
| | CFSVAALTNN | M9 | YSDVENPHLMG | M59 |
| 735 | AMHAASGNLLLDKRT | M50;M7; | FTRGVYYPDKVFRSS | M4;M12;M14;M1;M29;M72; |
| | TCFSVAALTN | M9 | VLHSTQDLFL | M67 |
| 736 | HAASGNLLLDKRTTCF | M50;M7; | SFTRGVYYPDKVFRS | M4;M12;M14;M1;M29;M72; |
| | SVAALTNNV | M9 | SVLHSTQDLF | M67 |
| 737 | SVCRLMKTIGPDMFLG | M6;M59; | VLTSHTVMPLSAPTL | M28;M54;M29;M45;M51; |
| | TCRRCPAEI | M37 | VPQEHYVRIT | M46;M39 |
| 738 | SYECDIPIGAGICASYQ | M31;M28; | IPYNSVTSSIVITSGDG | M28;M14;M12;M29;M45; |
| | TQTNSPRR | M70 | TTSPISEH | M62;M43 |
| 739 | SQCVNLTTRTQLPPAY | M33;M9; | EKALKYLPIDKCSRIIP | M13;M28;M14;M29;M51; |
| | TNSFTRGVY | M2 | ARARVECF | M33;M22 |
| 740 | VYDNKLKAHKDKSAQ | M13;M21; | CVVLHSYFTSDYYQL | M40;M3;M41;M43;M42; |
| | CFKMFYKGVI | M63 | YSTQLSTDTG | M39;M20 |
| 741 | HRFYRLANECAQVLSE | M79;M43; | RFDNPVLPFNDGVYF | M64;M12;M28;M24;M6; |
| | MVMCGGSLY | M28 | ASTEKSNIIR | M45;M22 |
| 742 | LKYAISAKNRARTVA | M35;M2; | DCLGDIAARDLICAQ | M58;M78;M65;M10;M3; |
| | GVSICSTMTN | M18 | KFNGLTVLPP | M79;M50 |
| 743 | NLKYAISAKNRARTV | M35;M2; | CLGDIAARDLICAQKF | M58;M78;M65;M10;M3; |
| | AGVSICSTMT | M18 | NGLTVLPPL | M79;M50 |
| 744 | VKQIYKTPPIKDFGGF | M33;M32; | DYFVLTSHTVMPLSA | M54;M60;M29;M45;M51; |
| | NFSQILPDP | M28 | PTLVPQEHYV | M46;M39 |
| 745 | VDLFRNARNGVLITEG | M33;M30; | LGDISGINASVVNIQK | M28;M14;M70;M80;M45; |
| | SVKGLQPSV | M22 | EIDRLNEVA | M35;M47 |
| 746 | QVDLFRNARNGVLITE | M33;M30; | DLGDISGINASVVNIQ | M28;M14;M70;M80;M45; |
| | GSVKGLQPS | M22 | KEIDRLNEV | M35;M47 |
| 747 | FNVLFSTVFPPTSFGPL | M71;M72; | ILGAGCFVDDIVKTD | M9;M10;M7;M6;M63;M8; |
| | VRKIFVDG | M54 | GTLMIERFVS | M11 |
| 748 | ASAFFGMSRIGMEVTP | M5;M28; | LGAGCFVDDIVKTDG | M9;M10;M7;M6;M63;M8; |
| | SGTWLTYTG | M23 | TLMIERFVSL | M11 |
| 749 | AFFGMSRIGMEVTPSG | M5;M28; | EIKDATPSDFVRATAT | M9;M14;M34;M45;M33;M8; |
| | TWLTYTGAI | M23 | IPIQASLPF | M11 |
| 750 | SAFFGMSRIGMEVTPS | M5;M28; | PFNDGVYFASTEKSNI | M64;M12;M24;M10;M6; |
| | GTWLTYTGA | M23 | IRGWIFGTT | M45;M22 |
| 751 | FFGMSRIGMEVTPSGT | M5;M28; | FNDGVYFASTEKSNII | M64;M12;M24;M10;M6; |
| | WLTYTGAIK | M23 | RGWIFGTTL | M45;M22 |
| 752 | DKCSRIIPARARVECFD | M33;M9; | NDGVYFASTEKSNIIR | M64;M12;M24;M10;M6; |
| | KFKVNSTL | M28 | GWIFGTTLD | M45;M22 |
| 753 | YANSVFNICQAVTAN | M11;M39; | VTPSGTWLTYTGAIK | M19;M10;M7;M41;M8;M35; |
| | VNALLSTDGN | M9 | LDDKDPNFKD | M11 |
| 754 | ANSVFNICQAVTANV | M11;M39; | EVTPSGTWLTYTGAI | M19;M10;M7;M41;M8;M35; |
| | NALLSTDGNK | M9 | KLDDKDPNFK | M11 |
| 755 | EVRQIAPGQTGKIADY | M80;M67; | SEETGTLIVNSVLLFL | M73;M14;M7;M50;M18; |
| | NYKLPDDFT | M38 | AFVVFLLVT | M11;M44 |
| 756 | IAPGQTGKIADYNYKL | M80;M67; | LFDMSKFPLKLRGTA | M54;M14;M60;M6;M67;M8; |
| | PDDFTGCVI | M38 | VMSLKEGQIN | M59 |
| 757 | RQIAPGQTGKIADYNY | M80;M67; | GVPVVDSYYSLLMPI | M58;M16;M14;M34;M8; |
| | KLPDDFTGC | M38 | LTLTRALTAE | M17;M39 |
| 758 | QIAPGQTGKIADYNYK | M80;M67; | TPTWRVYSTGSNVFQ | M75;M14;M5;M6;M41;M8; |
| | LPDDFTGCV | M38 | TRAGCLIGAE | M82 |
| 759 | VRQIAPGQTGKIADYN | M80;M67; | QLTPTWRVYSTGSNV | M75;M14;M5;M6;M41;M8; |
| | YKLPDDFTG | M38 | FQTRAGCLIG | M82 |
| 760 | QPYRVVVLSFELLHAP | M46;M34; | LTPTWRVYSTGSNVF | M75;M14;M5;M6;M41;M8; |
| | ATVCGPKKS | M39 | QTRAGCLIGA | M82 |
| 761 | RVVVLSFELLHAPATV | M46;M34; | ALPETTADIVVFDEIS | M28;M55;M7;M38;M48; |
| | CGPKKSTNL | M39 | MATNYDLSV | M33;M49 |
| 762 | PYRVVVLSFELLHAPA | M46;M34; | NALPETTADIVVFDEI | M28;M55;M7;M38;M48; |
| | TVCGPKKST | M39 | SMATNYDLS | M33;M49 |
| 763 | YQPYRVVVLSFELLHA | M46;M34; | LPETTADIVVFDEISM | M28;M55;M7;M38;M48; |
| | PATVCGPKK | M39 | ATNYDLSVV | M33;M49 |
| 764 | YRVVVLSFELLHAPAT | M46;M34; | YYSLLMPILTLTRALT | M12;M58;M16;M30;M8; |
| | VCGPKKSTN | M39 | AESHVDTDL | M17;M15 |
| 765 | VVVLSFELLHAPATVC | M46;M34; | SLLMPILTLTRALTAE | M12;M58;M16;M30;M8; |
| | GPKKSTNLV | M39 | SHVDTDLTK | M17;M15 |
| 766 | MKTIGPDMFLGTCRRC | M71;M16; | YSLLMPILTLTRALTA | M12;M58;M16;M30;M8; |
| | PAEIVDTVS | M37 | ESHVDTDLT | M17;M15 |
| 767 | TIGPDMFLGTCRRCPA | M71;M16; | SYYSLLMPILTLTRAL | M12;M58;M16;M30;M8; |
| | EIVDTVSAL | M37 | TAESHVDTD | M17;M15 |
| 768 | RLMKTIGPDMFLGTCR | M71;M16; | LMPILTLTRALTAESH | M12;M58;M16;M30;M8; |
| | RCPAEIVDT | M37 | VDTDLTKPY | M17;M15 |
| 769 | KTIGPDMFLGTCRRCP | M71;M16; | LLMPILTLTRALTAES | M12;M58;M16;M30;M8; |
| | AEIVDTVSA | M37 | HVDTDLTKP | M17;M15 |
| 770 | LMKTIGPDMFLGTCRR | M71;M16; | TVSALVYDNKLKAH | M13;M21;M28;M16;M7; |
| | CPAEIVDTV | M37 | KDKSAQCFKMF | M63;M17 |
| 771 | IGPDMFLGTCRRCPAEI | M71;M16; | DYILANTCTERLKLFA | M64;M40;M6;M48;M63; |
| | VDTVSALV | M37 | AETLKATEE | M49;M26 |
| 772 | GQVFGLYKNTCVGSD | M73;M28; | YILANTCTERLKLFAA | M64;M40;M6;M48;M63; |
| | NVTDFNAIAT | M59 | ETLKATEET | M49;M26 |
| 773 | VFGLYKNTCVGSDNV | M73;M28; | DDFVEIIKSQDLSVVS | M4;M48;M18;M8;M43;M15; |
| | TDFNAIATCD | M59 | KVVKVTIDY | M35 |
| 774 | NGQVFGLYKNTCVGS | M73;M28; | ARMAGNGGDAALAL | M13;M56;M58;M53;M51; |
| | DNVTDFNAIA | M59 | LLLDRLNQLES | M17;M11 |
| 775 | QVFGLYKNTCVGSDN | M73;M28; | SYGIATVREVLSDREL | M28;M7;M5;M6;M49;M11; |
| | VTDFNAIATC | M59 | HLSWEVGKP | M59 |
| 776 | VLSFELLHAPATVCGP | M46;M39; | LSYGIATVREVLSDRE | M28;M7;M5;M6;M49;M11; |
| | KKSTNLVKN | M27 | LHLSWEVGK | M59 |
| 777 | SPYNSQNAVASKILGL | M60;M39; | PSRILGAGCFVDDIVK | M9;M10;M6;M63;M8;M11; |
| | PTQTVDSSQ | M54 | TDGTLMIER | M20 |
| 778 | GAVILRGHLRIAGHHL | M55;M58; | GEVFNATRFASVYAW | M19;M16;M55;M25;M53; |
| | GRCDIKDLP | M17 | NRKRISNCVA | M2;M20 |
| 779 | TLPVNVAFELWAKRNI | M55;M66; | NDPFLGVYYHKNNKS | M4;M21;M28;M70;M1;M37; |
| | KPVPEVKIL | M74 | WMESEFRVYS | M22 |
| 780 | PVNVAFELWAKRNIKP | M55;M66; | CNDPFLGVYYHKNN | M4;M21;M28;M70;M1;M37; |
| | VPEVKILNN | M74 | KSWMESEFRVY | M22 |
| 781 | LPVNVAFELWAKRNI | M55;M66; | QFCNDPFLGVYYHKN | M4;M21;M28;M70;M1;M37; |
| | KPVPEVKILN | M74 | NKSWMESEFR | M22 |
| 782 | VNFNFNGLTGTGVLTE | M39;M38; | FCNDPFLGVYYHKNN | M4;M21;M28;M70;M1;M37; |
| | SNKKFLPFQ | M61 | KSWMESEFRV | M22 |
| 783 | CLVGLMWLSYFIASFR | M35;M26; | RLQSLENVAFNVVNK | M4;M71;M16;M24;M1;M27; |
| | LFARTRSMW | M16 | GHFDGQQGEV | M15 |
| 784 | ACLVGLMWLSYFIASF | M35;M26; | KPVPEVKILNNLGVDI | M9;M54;M48;M57;M27; |
| | RLFARTRSM | M16 | AANTVIWDY | M11;M44 |
| 785 | AARDLICAQKFNGLTV | M78;M39; | ISGINASVVNIQKEIDR | M28;M14;M70;M80;M35; |
| | LPPLLTDEM | M58 | LNEVAKNL | M82;M47 |
| 786 | GATVVIGTSKFYGGW | M18;M72; | LTGHMLDMYSVMLT | M64;M31;M7;M34;M61; |
| | HNMLKTVYSD | M17 | NDNTSRYWEPE | M36;M22 |
| 787 | RGATVVIGTSKFYGG | M18;M72; | GHMLDMYSVMLTND | M64;M31;M7;M34;M61; |
| | WHNMLKTVYS | M17 | NTSRYWEPEFY | M36;M22 |
| 788 | NEFYAYLRKHFSMMIL | M57;M13; | TGHMLDMYSVMLTN | M64;M31;M7;M34;M61; |
| | SDDAVVCFN | M71 | DNTSRYWEPEF | M36;M22 |
| 789 | NKCVNFNFNGLTGTG | M42;M39; | TPINLVRDLPQGFSAL | M64;M40;M31;M45;M36; |
| | VLTESNKKFL | M38 | EPLVDLPIG | M52;M47 |
| 790 | KNKCVNFNFNGLTGT | M42;M39; | FPLKLRGTAVMSLKE | M54;M14;M60;M6;M67; |
| | GVLTESNKKF | M38 | GQINDMILSL | M49;M59 |
| 791 | KCVNFNFNGLTGTGV | M42;M39; | KFPLKLRGTAVMSLK | M54;M14;M60;M6;M67; |
| | LTESNKKFLP | M38 | EGQINDMILS | M49;M59 |
| 792 | VKNKCVNFNFNGLTG | M42;M39; | DKFKVNSTLEQYVFC | M40;M29;M6;M44;M50; |
| | TGVLTESNKK | M38 | TVNALPETTA | M18;M47 |
| 793 | SFVIRGDEVRQIAPGQ | M33;M80; | PVVDSYYSLLMPILTL | M58;M16;M34;M8;M17; |
| | TGKIADYNY | M62 | TRALTAESH | M15;M39 |
| 794 | DSFVIRGDEVRQIAPG | M33;M80; | VPVVDSYYSLLMPILT | M58;M16;M34;M8;M17; |
| | QTGKIADYN | M62 | LTRALTAES | M15;M39 |
| 795 | LAMDEFIERYKLEGYA | M15;M58; | IDGYFKIYSKHTPINL | M64;M40;M71;M16;M36; |
| | FEHIVYGDF | M16 | VRDLPQGFS | M15;M52 |
| 796 | EIDFLELAMDEFIERYK | M15;M58; | NNTVYDPLQPELDSF | M70;M34;M6;M63;M72; |
| | LEGYAFEH | M16 | KEELDKYFKN | M47;M59 |
| 797 | MEIDFLELAMDEFIER | M15;M58; | NTVYDPLQPELDSFK | M70;M34;M6;M63;M72; |
| | YKLEGYAFE | M16 | EELDKYFKNH | M47;M59 |
| 798 | ELAMDEFIERYKLEGY | M15;M58; | YSFVSEETGTLIVNSV | M56;M73;M7;M50;M18; |
| | AFEHIVYGD | M16 | LLFLAFVVF | M11;M44 |
| 799 | FLELAMDEFIERYKLE | M15;M58; | FSVAALTNNVAFQTV | M73;M1;M25;M29;M6;M22; |
| | GYAFEHIVY | M16 | KPGNFNKDFY | M59 |
| 800 | IDFLELAMDEFIERYKL | M15;M58; | IMRTFKVSIWNLDYII | M56;M1;M5;M50;M18;M44; |
| | EGYAFEHI | M16 | NLIIKNLSK | M20 |
| 801 | AMDEFIERYKLEGYAF | M15;M58; | RKHFSMMILSDDAVV | M13;M21;M7;M53;M48; |
| | EHIVYGDFS | M16 | CFNSTYASQG | M49;M22 |
| 802 | LELAMDEFIERYKLEG | M15;M58; | KHFSMMILSDDAVVC | M13;M21;M7;M53;M48; |
| | YAFEHIVYG | M16 | FNSTYASQGL | M49;M22 |
| 803 | DFLELAMDEFIERYKL | M15;M58; | ALPQRQKKQQTVTLL | M54;M14;M60;M29;M46; |
| | EGYAFEHIV | M16 | PAADLDDFSK | M26;M39 |
| 804 | MACLVGLMWLSYFIA | M60;M26; | LPQRQKKQQTVTLLP | M54;M14;M60;M29;M46; |
| | SFRLFARTRS | M16 | AADLDDFSKQ | M26;M39 |
| 805 | AMACLVGLMWLSYFI | M60;M26; | QRQKKQQTVTLLPAA | M54;M14;M60;M29;M46; |
| | ASFRLFARTR | M16 | DLDDFSKQLQ | M26;M39 |
| 806 | VVIGIVNNTVYDPLQP | M34;M72; | PQRQKKQQTVTLLPA | M54;M14;M60;M29;M46; |
| | ELDSFKEEL | M47 | ADLDDFSKQL | M26;M39 |
| 807 | VIGIVNNTVYDPLQPE | M34;M72; | IPIGAGICASYQTQTN | M28;M19;M31;M70;M25; |
| | LDSFKEELD | M47 | SPRRARSVA | M50;M18 |
| 808 | DVVIGIVNNTVYDPLQ | M34;M72; | DIPIGAGICASYQTQT | M28;M19;M31;M70;M25; |
| | PELDSFKEE | M47 | NSPRRARSV | M50;M18 |
| 809 | GNCDVVIGIVNNTVYD | M34;M72; | LALSKGVHFVCNLLL | M6;M50;M18;M8;M49;M22; |
| | PLQPELDSF | M47 | LFVTVYSHLL | M23 |
| 810 | CDVVIGIVNNTVYDPL | M34;M72; | SKGVHFVCNLLLLFV | M21;M6;M50;M8;M43;M22; |
| | QPELDSFKE | M47 | TVYSHLLLVA | M23 |
| 811 | SGNCDVVIGIVNNTVY | M34;M72; | LSKGVHFVCNLLLLF | M21;M6;M50;M8;M43;M22; |
| | DPLQPELDS | M47 | VTVYSHLLLV | M23 |
| 812 | NCDVVIGIVNNTVYDP | M34;M72; | ADKYVRNLQHRLYE | M54;M60;M2;M37;M44; |
| | LQPELDSFK | M47 | CLYRNRDVDTD | M52;M47 |
| 813 | LLDKRTTCFSVAALTN | M73;M29; | REEAIRHVRAWIGFD | M64;M32;M14;M48;M57; |
| | NVAFQTVKP | M22 | VEGCHATREA | M69;M20 |
| 814 | LLLDKRTTCFSVAALT | M73;M29; | VTQQLIRAAEIRASAN | M64;M40;M21;M12;M48; |
| | NNVAFQTVK | M22 | LAATKMSEC | M22;M23 |
| 815 | YYKKVDGVVQQLPET | M71;M60; | NFYEPQIITTDNTFVS | M64;M12;M10;M7;M30;M9; |
| | YFTQSRNLQE | M54 | GNCDVVIGI | M23 |
| 816 | YKKVDGVVQQLPETY | M71;M60; | YEPQIITTDNTFVSGN | M64;M12;M10;M7;M30;M9; |
| | FTQSRNLQEF | M54 | CDVVIGIVN | M23 |
| 817 | EQDKNTQEVFAQVKQ | M55;M18; | EPQIITTDNTFVSGNC | M64;M12;M10;M7;M30;M9; |
| | IYKTPPIKDF | M56 | DVVIGIVNN | M23 |
| 818 | VEQDKNTQEVFAQVK | M55;M18; | FYEPQIITTDNTFVSG | M64;M12;M10;M7;M30;M9; |
| | QIYKTPPIKD | M56 | NCDVVIGIV | M23 |
| 819 | AVEQDKNTQEVFAQV | M55;M18; | RNFYEPQIITTDNTFV | M64;M12;M10;M7;M30;M9; |
| | KQIYKTPPIK | M56 | SGNCDVVIG | M23 |
| 820 | IAVEQDKNTQEVFAQ | M55;M18; | VQPTESIVRFPNITNLC | M21;M28;M66;M48;M41; |
| | VKQIYKTPPI | M56 | PFGEVFNA | M26;M22 |
| 821 | QDKNTQEVFAQVKQI | M55;M18; | RVQPTESIVRFPNITNL | M21;M28;M66;M48;M41; |
| | YKTPPIKDFG | M56 | CPFGEVFN | M26;M22 |
| 822 | KNTQEVFAQVKQIYK | M55;M18; | ALSKGVHFVCNLLLL | M6;M50;M18;M8;M43;M22; |
| | TPPIKDFGGF | M32 | FVTVYSHLLL | M23 |
| 823 | NTQEVFAQVKQIYKTP | M55;M18; | VTLADAGFIKQYGDC | M21;M70;M48;M57;M63; |
| | PIKDFGGFN | M32 | LGDIAARDLI | M41;M27 |
| 824 | QPRTFLLKYNENGTIT | M15;M11; | KVTLADAGFIKQYGD | M21;M70;M48;M57;M63; |
| | DAVDCALDP | M58 | CLGDIAARDL | M41;M27 |
| 825 | ADLVYALRHFDEGNC | M52;M17; | NKVTLADAGFIKQYG | M21;M70;M48;M57;M63; |
| | DTLKEILVTY | M49 | DCLGDIAARD | M41;M27 |
| 826 | INRPQIGVVREFLTRNP | M11;M73; | FNKVTLADAGFIKQY | M21;M70;M48;M57;M63; |
| | AWRKAVFI | M49 | GDCLGDIAAR | M41;M27 |
| 827 | RPQIGVVREFLTRNPA | M11;M73; | SNFGAISSVLNDILSRL | M58;M53;M6;M48;M50; |
| | WRKAVFISP | M49 | DKVEAEVQ | M17;M15 |
| 828 | NRPQIGVVREFLTRNP | M11;M73; | NFGAISSVLNDILSRL | M58;M53;M6;M48;M50; |
| | AWRKAVFIS | M49 | DKVEAEVQI | M17;M15 |
| 829 | SAINRPQIGVVREFLTR | M11;M73; | FGAISSVLNDILSRLD | M58;M53;M6;M48;M50; |
| | NPAWRKAV | M49 | KVEAEVQID | M17;M15 |
| 830 | AINRPQIGVVREFLTR | M11;M73; | LEDFIPMDSTVKNYFI | M19;M70;M25;M61;M18; |
| | NPAWRKAVF | M49 | TDAQTGSSK | M43;M39 |
| 831 | PQIGVVREFLTRNPAW | M11;M73; | EDFIPMDSTVKNYFIT | M19;M70;M25;M61;M18; |
| | RKAVFISPY | M49 | DAQTGSSKC | M43;M39 |
| 832 | SSAINRPQIGVVREFLT | M11;M73; | HKNNKSWMESEFRV | M64;M70;M5;M2;M48;M41; |
| | RNPAWRKA | M49 | YSSANNCTFEY | M82 |
| 833 | ANQFNSAIGKIQDSLSS | M79;M29; | FGDFIQTTPGSGVPVV | M40;M9;M58;M16;M34;M8; |
| | TASALGKL | M64 | DSYYSLLMP | M11 |
| 834 | TKHPNQEYADVFHLY | M71;M9; | IAQFAPSASAFFGMSR | M71;M28;M16;M70;M5; |
| | LQYIRKLHDE | M61 | IGMEVTPSG | M18;M17 |
| 835 | ALALLLLDRLNQLESK | M53;M11; | APSASAFFGMSRIGM | M71;M28;M16;M70;M5; |
| | MSGKGQQQQ | M58 | EVTPSGTWLT | M18;M17 |
| 836 | DQDALFAYTKRNVIPT | M55;M59; | AQFAPSASAFFGMSRI | M71;M28;M16;M70;M5; |
| | ITQMNLKYA | M10 | GMEVTPSGT | M18;M17 |
| 837 | VAGDSGFAAYSRYRIG | M71;M13; | QIAQFAPSASAFFGMS | M71;M28;M16;M70;M5; |
| | NYKLNTDHS | M41 | RIGMEVTPS | M18;M17 |
| 838 | QRVAGDSGFAAYSRY | M71;M13; | QFAPSASAFFGMSRIG | M71;M28;M16;M70;M5; |
| | RIGNYKLNTD | M41 | MEVTPSGTW | M18;M17 |
| 839 | GASQRVAGDSGFAAY | M71;M13; | FAPSASAFFGMSRIGM | M71;M28;M16;M70;M5; |
| | SRYRIGNYKL | M41 | EVTPSGTWL | M18;M17 |
| 840 | ASQRVAGDSGFAAYS | M71;M13; | NVTWFHAIHVSGTNG | M64;M40;M29;M45;M15; |
| | RYRIGNYKLN | M41 | TKRFDNPVLP | M22;M23 |
| 841 | RVAGDSGFAAYSRYRI | M71;M13; | TDGNKIADKYVRNLQ | M54;M60;M37;M57;M27; |
| | GNYKLNTDH | M41 | HRLYECLYRN | M44;M20 |
| 842 | SQRVAGDSGFAAYSR | M71;M13; | GAGICASYQTQTNSP | M19;M31;M70;M5;M25; |
| | YRIGNYKLNT | M41 | RRARSVASQS | M50;M18 |
| 843 | PETYFTQSRNLQEFKP | M71;M75; | AGICASYQTQTNSPRR | M19;M31;M70;M5;M25; |
| | RSQMEIDFL | M31 | ARSVASQSI | M50;M18 |
| 844 | NVAFELWAKRNIKPVP | M49;M66; | SNGTITVEELKKLLEQ | M4;M58;M16;M53;M17; |
| | EVKILNNLG | M74 | WNLVIGFLF | M52;M47 |
| 845 | VAFELWAKRNIKPVPE | M49;M66; | ENPDILRVYANLGER | M64;M11;M21;M32;M48; |
| | VKILNNLGV | M74 | VRQALLKTVQ | M49;M22 |
| 846 | NLNESLIDLQELGKYE | M17;M61; | SLSHRFYRLANECAQ | M28;M5;M79;M42;M43; |
| | QYIKWPWYI | M70 | VLSEMVMCGG | M46;M39 |
| 847 | NESLIDLQELGKYEQY | M17;M61; | LSHRFYRLANECAQV | M28;M5;M79;M42;M43; |
| | IKWPWYIWL | M70 | LSEMVMCGGS | M46;M39 |
| 848 | LNESLIDLQELGKYEQ | M17;M61; | QTVTLLPAADLDDFS | M71;M54;M58;M55;M53; |
| | YIKWPWYIW | M70 | KQLQQSMSSA | M26;M39 |
| 849 | VPEVKILNNLGVDIAA | M57;M9; | IRVGARKSAPLIELCV | M64;M40;M21;M7;M57; |
| | NTVIWDYKR | M27 | DEAGSKSPI | M22;M23 |
| 850 | CELYHYQECVRGTTV | M57;M27; | YIRVGARKSAPLIELC | M64;M40;M21;M7;M57; |
| | LLKEPCSSGT | M20 | VDEAGSKSP | M22;M23 |
| 851 | ELYHYQECVRGTTVL | M57;M27; | RVGARKSAPLIELCV | M64;M40;M21;M7;M57; |
| | LKEPCSSGTY | M20 | DEAGSKSPIQ | M22;M23 |
| 852 | TCELYHYQECVRGTT | M57;M27; | VGARKSAPLIELCVDE | M64;M40;M21;M7;M57; |
| | VLLKEPCSSG | M20 | AGSKSPIQY | M22;M23 |
| 853 | ATCELYHYQECVRGT | M57;M27; | WYIRVGARKSAPLIEL | M64;M40;M21;M7;M57; |
| | TVLLKEPCSS | M20 | CVDEAGSKS | M22;M23 |
| 854 | LSDRVVFVLWAHGFE | M46;M60; | SARIVYTACSHAAVD | M28;M60;M29;M38;M67; |
| | LTSMKYFVKI | M54 | ALCEKALKYL | M17;M49 |
| 855 | LKNLSDRVVFVLWAH | M46;M60; | AMPNLYKMQRMLLE | M12;M55;M53;M17;M52; |
| | GFELTSMKYF | M54 | KCDLQNYGDSA | M9;M23 |
| 856 | KNLSDRVVFVLWAHG | M46;M60; | LAFVVFLLVTLAILTA | M54;M16;M60;M25;M17; |
| | FELTSMKYFV | M54 | LRLCAYCCN | M46;M39 |
| 857 | NLSDRVVFVLWAHGF | M46;M60; | AFVVFLLVTLAILTAL | M54;M16;M60;M25;M17; |
| | ELTSMKYFVK | M54 | RLCAYCCNI | M46;M39 |
| 858 | DTLKNLSDRVVFVLW | M46;M60; | FVVFLLVTLAILTALR | M54;M16;M60;M25;M17; |
| | AHGFELTSMK | M54 | LCAYCCNIV | M46;M39 |
| 859 | TLKNLSDRVVFVLWA | M46;M60; | QHEETIYNLLKDCPA | M40;M30;M41;M42;M43; |
| | HGFELTSMKY | M54 | VAKHDFFKFR | M39;M11 |
| 860 | PLMYKGLPWNVVRIKI | M57;M9; | KIITLKKRWQLALSK | M11;M74;M33;M27;M8; |
| | VQMLSDTLK | M20 | GVHFVCNLLL | M42;M82 |
| 861 | GLTGTGVLTESNKKFL | M78;M24; | ATNYDLSVVNARLRA | M1;M25;M37;M57;M50; |
| | PFQQFGRDI | M61 | KHYVYIGDPA | M18;M26 |
| 862 | LTGTGVLTESNKKFLP | M78;M24; | ISAKNRARTVAGVSIC | M64;M32;M60;M30;M2; |
| | FQQFGRDIA | M61 | STMTNRQFH | M22;M23 |
| 863 | TGTGVLTESNKKFLPF | M78;M24; | TLKKRWQLALSKGV | M11;M74;M33;M8;M42; |
| | QQFGRDIAD | M61 | HFVCNLLLLFV | M82;M23 |
| 864 | RQRLTKYTMADLVYA | M10;M52; | KFKVNSTLEQYVFCT | M40;M29;M6;M18;M49; |
| | LRHFDEGNCD | M17 | VNALPETTAD | M35;M47 |
| 865 | ISRQRLTKYTMADLVY | M10;M52; | VNSTLEQYVFCTVNA | M40;M29;M6;M18;M49; |
| | ALRHFDEGN | M17 | LPETTADIVV | M35;M47 |
| 866 | SRQRLTKYTMADLVY | M10;M52; | FKVNSTLEQYVFCTV | M40;M29;M6;M18;M49; |
| | ALRHFDEGNC | M17 | NALPETTADI | M35;M47 |
| 867 | NFNGLTGTGVLTESNK | M24;M38; | STLEQYVFCTVNALP | M40;M29;M6;M18;M49; |
| | KFLPFQQFG | M61 | ETTADIVVFD | M35;M47 |
| 868 | NFNFNGLTGTGVLTES | M24;M38; | NSTLEQYVFCTVNAL | M40;M29;M6;M18;M49; |
| | NKKFLPFQQ | M61 | PETTADIVVF | M35;M47 |
| 869 | FNFNGLTGTGVLTESN | M24;M38; | KVNSTLEQYVFCTVN | M40;M29;M6;M18;M49; |
| | KKFLPFQQF | M61 | ALPETTADIV | M35;M47 |
| 870 | AIRHVRAWIGFDVEGC | M57;M69; | TLEQYVFCTVNALPE | M40;M29;M38;M6;M18; |
| | HATREAVGT | M20 | TTADIVVFDE | M49;M47 |
| 871 | EAIRHVRAWIGFDVEG | M57;M69; | VTQRNFYEPQIITTDN | M64;M10;M7;M30;M79;M9; |
| | CHATREAVG | M20 | TFVSGNCDV | M23 |
| 872 | RHVRAWIGFDVEGCH | M57;M69; | MILSDDAVVCFNSTY | M13;M21;M53;M48;M49; |
| | ATREAVGTNL | M20 | ASQGLVASIK | M26;M22 |
| 873 | IRHVRAWIGFDVEGCH | M57;M69; | MMILSDDAVVCFNST | M13;M21;M53;M48;M49; |
| | ATREAVGTN | M20 | YASQGLVASI | M26;M22 |
| 874 | GVYYHKNNKSWMES | M5;M41; | ILSDDAVVCFNSTYAS | M13;M21;M53;M48;M49; |
| | EFRVYSSANNC | M70 | QGLVASIKN | M26;M22 |
| 875 | FTIYSLLLCRMNSRNYI | M10;M54; | SMMILSDDAVVCFNS | M13;M21;M53;M48;M49; |
| | AQVDVVNF | M24 | TYASQGLVAS | M26;M22 |
| 876 | IYSLLLCRMNSRNYIA | M10;M54; | LSLLSKGRLIIRENNR | M11;M21;M24;M10;M48; |
| | QVDVVNFNL | M24 | VVISSDVLV | M41;M9 |
| 877 | AFPFTIYSLLLCRMNSR | M10;M54; | LLSKGRLIIRENNRVV | M11;M21;M24;M10;M48; |
| | NYIAQVDV | M24 | ISSDVLVNN | M41;M9 |
| 878 | FPFTIYSLLLCRMNSRN | M10;M54; | MILSLLSKGRLIIRENN | M11;M21;M24;M10;M48; |
| | YIAQVDVV | M24 | RVVISSDV | M41;M9 |
| 879 | PFTIYSLLLCRMNSRN | M10;M54; | SLLSKGRLIIRENNRV | M11;M21;M24;M10;M48; |
| | YIAQVDVVN | M24 | VISSDVLVN | M41;M9 |
| 880 | FAFPFTIYSLLLCRMNS | M10;M54; | ILSLLSKGRLIIRENNR | M11;M21;M24;M10;M48; |
| | RNYIAQVD | M24 | VVISSDVL | M41;M9 |
| 881 | TIYSLLLCRMNSRNYI | M10;M54; | GLMWLSYFIASFRLF | M64;M40;M16;M30;M15; |
| | AQVDVVNFN | M24 | ARTRSMWSFN | M35;M26 |
| 882 | VFAFPFTIYSLLLCRM | M10;M54; | TRFASVYAWNRKRIS | M16;M55;M25;M53;M74; |
| | NSRNYIAQV | M24 | NCVADYSVLY | M67;M27 |
| 883 | NVFAFPFTIYSLLLCR | M10;M54; | PQGLPNNTASWFTAL | M40;M28;M70;M29;M38; |
| | MNSRNYIAQ | M24 | TQHGKEDLKF | M18;M15 |
| 884 | VNRFNVAITRAKVGIL | M25;M68; | RRPQGLPNNTASWFT | M40;M28;M70;M29;M38; |
| | CIMSDRDLY | M66 | ALTQHGKEDL | M18;M15 |
| 885 | VITHDVSSAINRPQIGV | M49;M21; | RPQGLPNNTASWFTA | M40;M28;M70;M29;M38; |
| | VREFLTRN | M70 | LTQHGKEDLK | M18;M15 |
| 886 | AFDKSAFVNLKQLPFF | M60;M41; | QRRPQGLPNNTASWF | M40;M28;M70;M29;M38; |
| | YYSDSPCES | M54 | TALTQHGKED | M18;M15 |
| 887 | FDKSAFVNLKQLPFFY | M60;M41; | HFVCNLLLLFVTVYS | M21;M6;M37;M41;M43; |
| | YSDSPCESH | M54 | HLLLVAAGLE | M42;M22 |
| 888 | HFPREGVFVSNGTHW | M50;M18; | VHFVCNLLLLFVTVY | M21;M6;M37;M41;M43; |
| | FVTQRNFYEP | M24 | SHLLLVAAGL | M42;M22 |
| 889 | AHFPREGVFVSNGTH | M50;M18; | VNQNAQALNTLVKQ | M58;M16;M53;M17;M15; |
| | WFVTQRNFYE | M24 | LSSNFGAISSV | M46;M22 |
| 890 | SNVANYQKVGMQKY | M21;M34; | NQNAQALNTLVKQLS | M58;M16;M53;M17;M15; |
| | STLQGPPGTGK | M41 | SNFGAISSVL | M46;M22 |
| 891 | NGTKRFDNPVLPFNDG | M78;M65; | VDTDFVNEFYAYLRK | M71;M16;M18;M35;M15; |
| | VYFASTEKS | M82 | HFSMMILSDD | M52;M26 |
| 892 | LGSLVVRCSFYEDFLE | M21;M41; | DVDTDFVNEFYAYLR | M71;M16;M18;M35;M15; |
| | YHDVRVVLD | M70 | KHFSMMILSD | M52;M26 |
| 893 | GSLVVRCSFYEDFLEY | M21;M41; | LLLQYGSFCTQLNRA | M13;M28;M70;M41;M67; |
| | HDVRVVLDF | M70 | LTGIAVEQDK | M43;M20 |
| 894 | SLVVRCSFYEDFLEYH | M21;M41; | HTFSNYQHEETIYNLL | M40;M34;M30;M41;M27; |
| | DVRVVLDFI | M70 | KDCPAVAKH | M42;M11 |
| 895 | VDFQVTIAEILLIIMRT | M5;M61; | VPVAIHADQLTPTWR | M58;M24;M16;M5;M41; |
| | FKVSIWNL | M37 | VYSTGSNVFQ | M17;M82 |
| 896 | VLLPLVSSQCVNLTTR | M43;M9; | LVYFLQSINFVRIIMR | M4;M75;M24;M10;M25;M9; |
| | TQLPPAYTN | M24 | LWLCWKCRS | M23 |
| 897 | LVLLPLVSSQCVNLTT | M43;M9; | VLARKHTTCCSLSHR | M81;M5;M60;M2;M79;M43; |
| | RTQLPPAYT | M24 | FYRLANECAQ | M42 |
| 898 | PYNMRVIHFGAGSDK | M5;M13; | WFVTQRNFYEPQIITT | M64;M10;M5;M7;M79;M9; |
| | GVAPGTAVLR | M70 | DNTFVSGNC | M23 |
| 899 | LAVPYNMRVIHFGAG | M5;M13; | LPAADLDDFSKQLQQ | M71;M32;M58;M31;M55; |
| | SDKGVAPGTA | M70 | SMSSADSTQA | M53;M33 |
| 900 | VPYNMRVIHFGAGSD | M5;M13; | PNQEYADVFHLYLQY | M71;M32;M16;M61;M52; |
| | KGVAPGTAVL | M70 | IRKLHDELTG | M9;M47 |
| 901 | YNMRVIHFGAGSDKG | M5;M13; | EDQDALFAYTKRNVI | M71;M24;M55;M10;M35; |
| | VAPGTAVLRQ | M70 | PTITQMNLKY | M59;M23 |
| 902 | AVPYNMRVIHFGAGS | M5;M13; | LWAKRNIKPVPEVKIL | M54;M66;M74;M57;M27; |
| | DKGVAPGTAV | M70 | NNLGVDIAA | M49;M9 |
| 903 | TLLALHRSYLTPGDSS | M5;M75; | HAIHVSGTNGTKRFD | M64;M40;M22;M78;M65; |
| | SGWTAGAAA | M70 | NPVLPFNDGV | M82;M23 |
| 904 | AGSDKGVAPGTAVLR | M13;M26; | FHAIHVSGTNGTKRF | M64;M40;M22;M78;M65; |
| | QWLPTGTLLV | M70 | DNPVLPFNDG | M82;M23 |
| 905 | TITQMNLKYAISAKNR | M35;M82; | IDSYFVVKRHTFSNY | M71;M58;M18;M15;M52; |
| | ARTVAGVSI | M18 | QHEETIYNLL | M47;M20 |
| 906 | PTITQMNLKYAISAKN | M35;M82; | VSNGTHWFVTQRNFY | M24;M31;M5;M79;M50; |
| | RARTVAGVS | M18 | EPQIITTDNT | M18;M35 |
| 907 | ITQMNLKYAISAKNRA | M35;M82; | SNGTHWFVTQRNFYE | M24;M31;M5;M79;M50; |
| | RTVAGVSIC | M18 | PQIITTDNTF | M18;M35 |
| 908 | FNVAITRAKVGILCIMS | M32;M68; | FVSNGTHWFVTQRNF | M24;M31;M5;M79;M50; |
| | DRDLYDKL | M66 | YEPQIITTDN | M18;M35 |
| 909 | VAITRAKVGILCIMSD | M32;M68; | LPGCDGGSLYVNKHA | M56;M73;M58;M24;M50; |
| | RDLYDKLQF | M66 | FHTPAFDKSA | M11;M47 |
| 910 | RENVAITRAKVGILCI | M32;M68; | GCDGGSLYVNKHAF | M56;M73;M58;M24;M50; |
| | MSDRDLYDK | M66 | HTPAFDKSAFV | M11;M47 |
| 911 | NVAITRAKVGILCIMS | M32;M68; | LNLPGCDGGSLYVNK | M56;M73;M58;M24;M50; |
| | DRDLYDKLQ | M66 | HAFHTPAFDK | M11;M47 |
| 912 | LYLQYIRKLHDELTGH | M52;M32; | NLNLPGCDGGSLYVN | M56;M73;M58;M24;M50; |
| | MLDMYSVML | M38 | KHAFHTPAFD | M11;M47 |
| 913 | DYYRYNLPTMCDIRQ | M70;M74; | PGCDGGSLYVNKHAF | M56;M73;M58;M24;M50; |
| | LLFVVEVVDK | M20 | HTPAFDKSAF | M11;M47 |
| 914 | YYRYNLPTMCDIRQLL | M70;M74; | NLPGCDGGSLYVNKH | M56;M73;M58;M24;M50; |
| | FVVEVVDKY | M20 | AFHTPAFDKS | M11;M47 |
| 915 | INFVRIIMRLWLCWKC | M75;M76; | GTHWFVTQRNFYEPQ | M24;M31;M5;M79;M50; |
| | RSKNPLLYD | M9 | IITTDNTFVS | M18;M9 |
| 916 | NFVRIIMRLWLCWKC | M75;M76; | SPFELEDFIPMDSTVK | M4;M19;M1;M25;M3;M61; |
| | RSKNPLLYDA | M9 | NYFITDAQT | M18;M43 |
| 917 | KRHTFSNYQHEETIYN | M34;M27; | RFKESPFELEDFIPMD | M4;M19;M1;M25;M3;M61; |
| | LLKDCPAVA | M20 | STVKNYFIT | M18;M43 |
| 918 | VKRHTFSNYQHEETIY | M34;M27; | FKESPFELEDFIPMDS | M4;M19;M1;M25;M3;M61; |
| | NLLKDCPAV | M20 | TVKNYFITD | M18;M43 |
| 919 | VVKRHTFSNYQHEETI | M34;M27; | ESPFELEDFIPMDSTV | M4;M19;M1;M25;M3;M61; |
| | YNLLKDCPA | M20 | KNYFITDAQ | M18;M43 |
| 920 | ECVRGTTVLLKEPCSS | M27;M58; | KESPFELEDFIPMDST | M4;M19;M1;M25;M3;M61; |
| | GTYEGNSPF | M20 | VKNYFITDA | M18;M43 |
| 921 | CVRGTTVLLKEPCSSG | M27;M58; | GLAKRFKESPFELEDF | M4;M19;M1;M25;M3;M18; |
| | TYEGNSPFH | M20 | IPMDSTVKN | M43;M20 |
| 922 | NGYPNMFITREEAIRH | M64;M69; | NLCPFGEVFNATRFAS | M4;M19;M58;M25;M3;M2; |
| | VRAWIGFDV | M32 | VYAWNRKRI | M17;M20 |
| 923 | ISTIGVCSMTDIAKKPT | M9;M59; | TNLCPFGEVFNATRF | M4;M19;M58;M25;M3;M2; |
| | ETICAPLT | M70 | ASVYAWNRKR | M17;M20 |
| 924 | HISTIGVCSMTDIAKKP | M9;M59; | LCPFGEVFNATRFASV | M4;M19;M58;M25;M3;M2; |
| | TETICAPL | M70 | YAWNRKRIS | M17;M20 |
| 925 | TIGVCSMTDIAKKPTE | M9;M59; | FTRLQSLENVAFNVV | M4;M54;M16;M24;M1;M3; |
| | TICAPLTVF | M70 | NKGHFDGQQG | M27;M15 |
| 926 | STIGVCSMTDIAKKPT | M9;M59; | TSIKWADNNCYLATA | M4;M51;M3;M23;M4;M51; |
| | ETICAPLTV | M70 | LLTLQQIELK | M3;M23 |
| 927 | IAGLIAIVMVTIMLCC | M60;M69; | LTSIKWADNNCYLAT | M4;M51;M3;M23;M4;M51; |
| | MTSCCSCLK | M42 | ALLTLQQIEL | M3;M23 |
| 928 | VMVTIMLCCMTSCCS | M60;M42; | GLTSIKWADNNCYLA | M4;M51;M3;M23;M4;M51; |
| | CLKGCCSCGS | M37 | TALLTLQQIE | M3;M23 |
| 929 | IVMVTIMLCCMTSCCS | M60;M42; | LKSEDAQGMDNLAC | M4;M1;M3;M37;M4;M1;M3; |
| | CLKGCCSCG | M37 | EDLKPVSEEVV | M37 |
| 930 | AIVMVTIMLCCMTSCC | M60;M42; | YLYALVYFLQSINFVR | M4;M54;M24;M19;M60; |
| | SCLKGCCSC | M37 | IIMRLWLCW | M25;M3;M9 |
| 931 | GLIAIVMVTIMLCCMT | M60;M42; | GCFVDDIVKTDGTLM | M9;M10;M7;M5;M3;M6;M8; |
| | SCCSCLKGC | M37 | IERFVSLAID | M11 |
| 932 | IAIVMVTIMLCCMTSC | M60;M42; | RAAEIRASANLAATK | M4;M64;M40;M3;M48;M27; |
| | CSCLKGCCS | M37 | MSECVLGQSK | M22;M23 |
| 933 | MVTIMLCCMTSCCSCL | M60;M42; | IRAAEIRASANLAATK | M4;M64;M40;M3;M48;M27; |
| | KGCCSCGSC | M37 | MSECVLGQS | M22;M23 |
| 934 | GFIAGLIAIVMVTIMLC | M60;M69; | FDNPVLPFNDGVYFA | M64;M12;M14;M28;M24; |
| | CMTSCCSC | M42 | STEKSNIIRG | M6;M45;M22 |
| 935 | AGLIAIVMVTIMLCCM | M60;M42; | TRGVYYPDKVFRSSV | M4;M12;M14;M1;M29;M6; |
| | TSCCSCLKG | M37 | LHSTQDLFLP | M67;M72 |
| 936 | LIAIVMVTIMLCCMTS | M60;M42; | YYPDKVFRSSVLHST | M4;M12;M14;M1;M29;M6; |
| | CCSCLKGCC | M37 | QDLFLPFFSN | M67;M72 |
| 937 | FIAGLIAIVMVTIMLCC | M60;M69; | RGVYYPDKVFRSSVL | M4;M12;M14;M1;M29;M6; |
| | MTSCCSCL | M42 | HSTQDLFLPF | M67;M72 |
| 938 | DHVISTSHKLVLSVNP | M64;M10; | GVYYPDKVFRSSVLH | M4;M12;M14;M1;M29;M6; |
| | YVCNAPGCD | M23 | STQDLFLPFF | M67;M72 |
| 939 | LDDRCILHCANFNVLF | M4;M1;M3; | VYYPDKVFRSSVLHS | M4;M12;M14;M1;M29;M6; |
| | STVFPPTSF | M48 | TQDLFLPFFS | M67;M72 |
| 940 | QTVDSSQGSEYDYVIF | M4;M22; | YPDKVFRSSVLHSTQ | M4;M12;M14;M1;M29;M6; |
| | TQTTETAHS | M3;M51 | DLFLPFFSNV | M67;M72 |
| 941 | KTDGTLMIERFVSLAI | M1;M5;M2; | NYLKRRVVFNGVSFS | M4;M3;M6;M49;M4;M3; |
| | DAYPLTKHP | M3 | TFEEAALCTF | M6;M49 |
| 942 | TDGTLMIERFVSLAID | M1;M5;M2; | SNYLKRRVVFNGVSF | M4;M3;M6;M49;M4;M3;M6; |
| | AYPLTKHPN | M3 | STFEEAALCT | M49 |
| 943 | IKVCEFQFCNDPFLGV | M4;M1;M3; | LKRRVVFNGVSFSTFE | M4;M3;M6;M49;M4;M3;M6; |
| | YYHKNNKSW | M37 | EAALCTFLL | M49 |
| 944 | LIGLAKRFKESPFELED | M4;M1;M3; | YLKRRVVFNGVSFST | M4;M3;M6;M49;M4;M3;M6; |
| | FIPMDSTV | M20 | FEEAALCTFL | M49 |
| 945 | KVCEFQFCNDPFLGVY | M4;M1;M3; | GVVQLTSQWLTNIFG | M4;M45;M3;M59;M4;M45; |
| | YHKNNKSWM | M37 | TVYEKLKPVL | M3;M59 |
| 946 | HLLIGLAKRFKESPFEL | M4;M1;M3; | YALVYFLQSINFVRII | M4;M54;M24;M60;M25;M3; |
| | EDFIPMDS | M20 | MRLWLCWKC | M9;M23 |
| 947 | VIKVCEFQFCNDPFLG | M4;M1;M3; | KELLVYAADPAMHA | M12;M24;M48;M33;M36; |
| | VYYHKNNKS | M37 | ASGNLLLDKRT | M8;M46;M39 |
| 948 | IGLAKRFKESPFELEDF | M4;M1;M3; | PSKLIEYTDFATSACV | M29;M3;M14;M62;M29;M3; |
| | IPMDSTVK | M20 | LAAECTIFK | M14;M62 |
| 949 | LLIGLAKRFKESPFELE | M4;M1;M3; | YFVLTSHTVMPLSAP | M28;M54;M60;M29;M45; |
| | DFIPMDST | M20 | TLVPQEHYVR | M51;M46;M39 |
| 950 | GTYEGNSPFHPLADNK | M1;M3;M44; | FVLTSHTVMPLSAPTL | M28;M54;M60;M29;M45; |
| | FALTCFSTQ | M70 | VPQEHYVRI | M51;M46;M39 |
| 951 | GNSPFHPLADNKFALT | M1;M22;M3; | GDAALALLLLDRLNQ | M56;M58;M10;M53;M51; |
| | CFSTQFAFA | M14 | LESKMSGKGQ | M36;M17;M11 |
| 952 | EGNSPFHPLADNKFAL | M1;M3;M14; | ALCEKALKYLPIDKCS | M71;M13;M28;M14;M29; |
| | TCFSTQFAF | M70 | RIIPARARV | M51;M18;M22 |
| 953 | SPFHPLADNKFALTCF | M4;M22;M3; | DALCEKALKYLPIDK | M71;M13;M28;M14;M29; |
| | STQFAFACP | M14 | CSRIIPARAR | M51;M18;M22 |
| 954 | NITNLCPFGEVFNATR | M4;M3;M17; | AKSASVYYSQLMCQP | M51;M7;M60;M36;M51;M7; |
| | FASVYAWNR | M58 | ILLLDQALVS | M60;M36 |
| 955 | HTPAFDKSAFVNLKQL | M4;M60; | SASVYYSQLMCQPILL | M51;M7;M60;M36;M51;M7; |
| | PFFYYSDSP | M54;M3 | LDQALVSDV | M60;M36 |
| 956 | VNKHAFHTPAFDKSAF | M4;M60; | ASVYYSQLMCQPILL | M51;M7;M60;M36;M51;M7; |
| | VNLKQLPFF | M54;M3 | LDQALVSDVG | M60;M36 |
| 957 | FHTPAFDKSAFVNLKQ | M4;M60; | KSASVYYSQLMCQPI | M51;M7;M60;M36;M51;M7; |
| | LPFFYYSDS | M54;M3 | LLLDQALVSD | M60;M36 |
| 958 | KHAFHTPAFDKSAFVN | M4;M60; | KALKYLPIDKCSRIIPA | M13;M28;M14;M29;M51; |
| | LKQLPFFYY | M54;M3 | RARVECFD | M33;M26;M22 |
| 959 | NKHAFHTPAFDKSAFV | M4;M60; | VVTTVMFLARGIVFM | M12;M3;M22;M20;M12;M3; |
| | NLKQLPFFY | M54;M3 | CVEYCPIFFI | M22;M20 |
| 960 | TPAFDKSAFVNLKQLP | M4;M60; | GVVTTVMFLARGIVF | M12;M3;M22;M20;M12;M3; |
| | FFYYSDSPC | M54;M3 | MCVEYCPIFF | M22;M20 |
| 961 | HAFHTPAFDKSAFVNL | M4;M60; | TVMFLARGIVFMCVE | M12;M3;M22;M20;M12;M3; |
| | KQLPFFYYS | M54;M3 | YCPIFFITGN | M22;M20 |
| 962 | AFHTPAFDKSAFVNLK | M4;M60; | VMFLARGIVFMCVEY | M12;M3;M22;M20;M12;M3; |
| | QLPFFYYSD | M54;M3 | CPIFFITGNT | M22;M20 |
| 963 | ADYSVLYNSASFSTFK | M15;M41; | VTTVMFLARGIVFMC | M12;M3;M22;M20;M12;M3; |
| | CYGVSPTKL | M3;M44 | VEYCPIFFIT | M22;M20 |
| 964 | VADYSVLYNSASFSTF | M15;M41; | TTVMFLARGIVFMCV | M12;M3;M22;M20;M12;M3; |
| | KCYGVSPTK | M3;M44 | EYCPIFFITG | M22;M20 |
| 965 | VVIGTSKFYGGWHNM | M18;M72; | TEEVGHTDLMAAYV | M51;M63;M44;M70;M51; |
| | LKTVYSDVEN | M3;M6 | DNSSLTIKKPN | M63;M44;M70 |
| 966 | GTLIVNSVLLFLAFVV | M50;M73; | EVGHTDLMAAYVDN | M51;M63;M44;M70;M51; |
| | FLLVTLAIL | M3;M44 | SSLTIKKPNEL | M63;M44;M70 |
| 967 | TGTLIVNSVLLFLAFV | M50;M73; | EEVGHTDLMAAYVD | M51;M63;M44;M70;M51; |
| | VFLLVTLAI | M3;M44 | NSSLTIKKPNE | M63;M44;M70 |
| 968 | PAQLPAPRTLLTKGTL | M29;M3; | VGHTDLMAAYVDNS | M51;M63;M44;M70;M51; |
| | EPEYFNSVC | M6;M58 | SLTIKKPNELS | M63;M44;M70 |
| 969 | GTSKFYGGWHNMLKT | M6;M18; | IMQLFFSYFAVHFISN | M9;M3;M45;M23;M9;M3; |
| | VYSDVENPHL | M3;M59 | SWLMWLIIN | M45;M23 |
| 970 | IGTSKFYGGWHNMLK | M6;M18; | VEEVTTTLEETKFLTE | M50;M3;M6;M20;M50;M3; |
| | TVYSDVENPH | M3;M59 | NLLLYIDIN | M6;M20 |
| 971 | TSPDVDLGDISGINASV | M14;M80; | RGIVFMCVEYCPIFFIT | M40;M39;M11;M3;M40; |
| | VNIQKEID | M28;M45 | GNTLQCIM | M39;M11;M3 |
| 972 | HTSPDVDLGDISGINAS | M14;M80; | GIVFMCVEYCPIFFITG | M40;M39;M11;M3;M40; |
| | VVNIQKEI | M28;M45 | NTLQCIML | M39;M11;M3 |
| 973 | CLPFTINCQEPKLGSLV | M50;M29; | FLARGIVFMCVEYCPI | M39;M22;M3;M20;M39; |
| | VRCSFYED | M51;M14 | FFITGNTLQ | M22;M3;M20 |
| 974 | SCLPFTINCQEPKLGSL | M50;M29; | LARGIVFMCVEYCPIF | M39;M22;M3;M20;M39; |
| | VVRCSFYE | M51;M14 | FITGNTLQC | M22;M3;M20 |
| 975 | SHTVMPLSAPTLVPQE | M51;M69; | KRAKVTSAMQTMLF | M17;M52;M3;M16;M17; |
| | HYVRITGLY | M39;M28 | TMLRKLDNDAL | M52;M3;M16 |
| 976 | SEYDYVIFTQTTETAH | M51;M22; | KGIYQTSNFRVQPTES | M31;M34;M30;M48;M50; |
| | SCNVNRFNV | M6;M59 | IVRFPNITN | M51;M41;M39 |
| 977 | YDYVIFTQTTETAHSC | M51;M22; | IYQTSNFRVQPTESIV | M31;M34;M30;M48;M50; |
| | NVNRFNVAI | M6;M59 | RFPNITNLC | M51;M41;M39 |
| 978 | EYDYVIFTQTTETAHS | M51;M22; | YQTSNFRVQPTESIVR | M31;M34;M30;M48;M50; |
| | CNVNRFNVA | M6;M59 | FPNITNLCP | M51;M41;M39 |
| 979 | TACSHAAVDALCEKA | M51;M1; | GIYQTSNFRVQPTESI | M31;M34;M30;M48;M50; |
| | LKYLPIDKCS | M60;M49 | VRFPNITNL | M51;M41;M39 |
| 980 | FRKSNLKPFERDISTEI | M36;M53; | VTALRANSAVKLQNN | M51;M11;M73;M44;M51; |
| | YQAGSTPC | M17;M44 | ELSPVALRQM | M11;M73;M44 |
| 981 | LFRKSNLKPFERDISTE | M36;M53; | AVHFISNSWLMWLIIN | M50;M11;M73;M3;M50; |
| | IYQAGSTP | M17;M44 | LVQMAPISA | M11;M73;M3 |
| 982 | RLFRKSNLKPFERDIST | M36;M53; | TFYLTNDVSFLAHIQ | M60;M54;M3;M58;M60; |
| | EIYQAGST | M17;M44 | WMVMFTPLVP | M54;M3;M58 |
| 983 | YRLFRKSNLKPFERDIS | M36;M53; | DPSRILGAGCFVDDIV | M12;M10;M6;M57;M63;M8; |
| | TEIYQAGS | M17;M44 | KTDGTLMIE | M9;M20 |
| 984 | RKSNLKPFERDISTEIY | M36;M53; | EPEYFNSVCRLMKTIG | M12;M14;M16;M5;M6;M17; |
| | QAGSTPCN | M17;M44 | PDMFLGTCR | M15;M59 |
| 985 | DLATNNLVVMAYITG | M51;M12; | LEPEYFNSVCRLMKTI | M13;M12;M16;M14;M5;M6; |
| | GVVQLTSQWL | M51;M12 | GPDMFLGTC | M17;M15 |
| 986 | NSFTRGVYYPDKVFRS | M4;M1; | SEDKRAKVTSAMQT | M38;M52;M3;M62;M38; |
| | SVLHSTQDL | M29;M12 | MLFTMLRKLDN | M52;M3;M62 |
| 987 | FEKGDYGDAVVYRGT | M9;M12; | GTCRRCPAEIVDTVSA | M12;M7;M29;M6;M38;M8; |
| | TTYKLNVGDY | M8;M44 | LVYDNKLKA | M59;M23 |
| 988 | EKGDYGDAVVYRGTT | M9;M12; | LGTCRRCPAEIVDTVS | M12;M7;M29;M6;M38;M8; |
| | TYKLNVGDYF | M8;M44 | ALVYDNKLK | M59;M23 |
| 989 | WTIEYPIIGDELKINAA | M73;M19; | TCRRCPAEIVDTVSAL | M12;M7;M29;M6;M38;M8; |
| | CRKVQHMV | M17;M44 | VYDNKLKAH | M59;M23 |
| 990 | YPIIGDELKINAACRKV | M73;M19; | CRRCPAEIVDTVSALV | M12;M7;M29;M6;M38;M8; |
| | QHMVVKAA | M17;M44 | YDNKLKAHK | M59;M23 |
| 991 | IEYPIIGDELKINAACR | M73;M19; | YDYCIPYNSVTSSIVIT | M12;M14;M29;M38;M45; |
| | KVQHMVVK | M17;M44 | SGDGTTSP | M43;M62;M42 |
| 992 | EYPIIGDELKINAACRK | M73;M19; | NCYDYCIPYNSVTSSI | M12;M14;M29;M38;M45; |
| | VQHMVVKA | M17;M44 | VITSGDGTT | M43;M62;M42 |
| 993 | TIEYPIIGDELKINAAC | M73;M19; | CYDYCIPYNSVTSSIVI | M12;M14;M29;M38;M45; |
| | RKVQHMVV | M17;M44 | TSGDGTTS | M43;M62;M42 |
| 994 | PIIGDELKINAACRKVQ | M73;M19; | DYCIPYNSVTSSIVITS | M12;M14;M29;M38;M45; |
| | HMVVKAAL | M17;M44 | GDGTTSPI | M43;M62;M42 |
| 995 | TLVKQLSSNFGAISSVL | M34;M48; | TNCYDYCIPYNSVTSS | M12;M14;M29;M38;M45; |
| | NDILSRLD | M6;M45 | IVITSGDGT | M43;M62;M42 |
| 996 | NTLVKQLSSNFGAISS | M34;M48; | YCIPYNSVTSSIVITSG | M12;M14;M29;M38;M45; |
| | VLNDILSRL | M6;M45 | DGTTSPIS | M43;M62;M42 |
| 997 | AVASKILGLPTQTVDS | M51;M21; | IQKEIDRLNEVAKNLN | M11;M78;M65;M2;M79; |
| | SQGSEYDYV | M11;M54 | ESLIDLQEL | M51;M67;M82 |
| 998 | NAVASKILGLPTQTVD | M51;M21; | QKEIDRLNEVAKNLN | M11;M78;M65;M2;M79; |
| | SSQGSEYDY | M11;M54 | ESLIDLQELG | M51;M67;M82 |
| 999 | VDALCEKALKYLPIDK | M51;M18; | NIQKEIDRLNEVAKNL | M11;M78;M65;M2;M79; |
| | CSRIIPARA | M22;M71 | NESLIDLQE | M51;M67;M82 |
| 1000 | AQCFKMFYKGVITHD | M36;M11; | KDATPSDFVRATATIP | M9;M14;M34;M29;M45; |
| | VSSAINRPQI | M45;M70 | IQASLPFGW | M33;M8;M11 |
| 1001 | IWDYKRDAPAHISTIG | M36;M11; | IKDATPSDFVRATATI | M9;M14;M34;M29;M45; |
| | VCSMTDIAK | M6;M70 | PIQASLPFG | M33;M8;M11 |
| 1002 | NQVIVNNLDKSAGFPF | M33;M51; | DATPSDFVRATATIPI | M9;M14;M34;M29;M45; |
| | NKWGKARLY | M58;M17 | QASLPFGWL | M33;M8;M11 |
| 1003 | ESSAKSASVYYSQLMC | M57;M51; | DPAMHAASGNLLLD | M12;M7;M57;M50;M33;M8; |
| | QPILLLDQA | M57;M51 | KRTTCFSVAAL | M39;M9 |
| 1004 | KCEESSAKSASVYYSQ | M57;M51; | AADPAMHAASGNLL | M12;M7;M57;M50;M33;M8; |
| | LMCQPILLL | M57;M51 | LDKRTTCFSVA | M39;M9 |
| 1005 | KSKCEESSAKSASVYY | M57;M51; | ADPAMHAASGNLLL | M12;M7;M57;M50;M33;M8; |
| | SQLMCQPIL | M57;M51 | DKRTTCFSVAA | M39;M9 |
| 1006 | CEESSAKSASVYYSQL | M57;M51; | ADQLTPTWRVYSTGS | M75;M14;M16;M5;M6;M41; |
| | MCQPILLLD | M57;M51 | NVFQTRAGCL | M8;M82 |
| 1007 | GKSKCEESSAKSASVY | M57;M51; | DQLTPTWRVYSTGSN | M75;M14;M16;M5;M6;M41; |
| | YSQLMCQPI | M57;M51 | VFQTRAGCLI | M8;M82 |
| 1008 | SKCEESSAKSASVYYS | M57;M51; | MSKFPLKLRGTAVMS | M54;M14;M60;M6;M67;M8; |
| | QLMCQPILL | M57;M51 | LKEGQINDMI | M49;M59 |
| 1009 | EESSAKSASVYYSQLM | M57;M51; | FDMSKFPLKLRGTAV | M54;M14;M60;M6;M67;M8; |
| | CQPILLLDQ | M57;M51 | MSLKEGQIND | M49;M59 |
| 1010 | KSAQCFKMFYKGVIT | M52;M36; | DMSKFPLKLRGTAVM | M54;M14;M60;M6;M67;M8; |
| | HDVSSAINRP | M45;M70 | SLKEGQINDM | M49;M59 |
| 1011 | CYDGGCINANQVIVN | M50;M73; | SKFPLKLRGTAVMSL | M54;M14;M60;M6;M67;M8; |
| | NLDKSAGFPF | M17;M51 | KEGQINDMIL | M49;M59 |
| 1012 | HTPINLVRDLPQGFSA | M64;M40; | VSEETGTLIVNSVLLF | M56;M73;M14;M7;M50; |
| | LEPLVDLPI | M45;M36 | LAFVVFLLV | M18;M11;M44 |
| 1013 | KHTPINLVRDLPQGFS | M64;M40; | FVSEETGTLIVNSVLL | M56;M73;M14;M7;M50; |
| | ALEPLVDLP | M45;M36 | FLAFVVFLL | M18;M11;M44 |
| 1014 | YKGVITHDVSSAINRP | M36;M21; | SFVSEETGTLIVNSVL | M56;M73;M14;M7;M50; |
| | QIGVVREFL | M45;M70 | LFLAFVVFL | M18;M11;M44 |
| 1015 | DNINLHTQVVDMSMT | M51;M67; | TDLTKGPHEFCSQHT | M14;M29;M30;M6;M18; |
| | YGQQFGPTYL | M51;M67 | MLVKQGDDYV | M43;M42;M39 |
| 1016 | VDNINLHTQVVDMSM | M51;M67; | TKGPHEFCSQHTMLV | M14;M29;M30;M6;M18; |
| | TYGQQFGPTY | M51;M67 | KQGDDYVYLP | M43;M42;M39 |
| 1017 | QRKYKGIKIQEGVVDY | M51;M67; | DLTKGPHEFCSQHTM | M14;M29;M30;M6;M18; |
| | GARFYFYTS | M51;M67 | LVKQGDDYVY | M43;M42;M39 |
| 1018 | KYKGIKIQEGVVDYG | M51;M67; | ETDLTKGPHEFCSQH | M14;M29;M30;M6;M18; |
| | ARFYFYTSKT | M51;M67 | TMLVKQGDDY | M43;M42;M39 |
| 1019 | TIKVFTTVDNINLHTQ | M51;M67; | LTKGPHEFCSQHTML | M14;M29;M30;M6;M18; |
| | VVDMSMTYG | M51;M67 | VKQGDDYVYL | M43;M42;M39 |
| 1020 | RKYKGIKIQEGVVDYG | M51;M67; | LWVYKQFDTYNLWN | M71;M58;M16;M14;M19; |
| | ARFYFYTSK | M51;M67 | TFTRLQSLENV | M25;M17;M15 |
| 1021 | NSQNAVASKILGLPTQ | M51;M60; | YVTQQLIRAAEIRASA | M64;M40;M21;M12;M14; |
| | TVDSSQGSE | M21;M54 | NLAATKMSE | M48;M22;M23 |
| 1022 | PYNSQNAVASKILGLP | M51;M60; | LQTYVTQQLIRAAEIR | M64;M40;M21;M12;M14; |
| | TQTVDSSQG | M21;M54 | ASANLAATK | M48;M22;M23 |
| 1023 | YNSQNAVASKILGLPT | M51;M60; | TYVTQQLIRAAEIRAS | M64;M40;M21;M12;M14; |
| | QTVDSSQGS | M21;M54 | ANLAATKMS | M48;M22;M23 |
| 1024 | SQNAVASKILGLPTQT | M51;M60; | QTYVTQQLIRAAEIRA | M64;M40;M21;M12;M14; |
| | VDSSQGSEY | M21;M54 | SANLAATKM | M48;M22;M23 |
| 1025 | VNLHSSRLSFKELLVY | M57;M36; | AGLPYGANKDGIIWV | M64;M40;M14;M30;M6; |
| | AADPAMHAA | M48;M20 | ATEGALNTPK | M57;M49;M22 |
| 1026 | CTMYICGDSTECSNLL | M48;M39; | YSLRLIDAMMFTSDL | M57;M36;M50;M44;M57; |
| | LQYGSFCTQ | M45;M41 | ATNNLVVMAY | M36;M50;M44 |
| 1027 | LLKSIAATRGATVVIG | M17;M72; | QYSLRLIDAMMFTSD | M57;M36;M50;M44;M57; |
| | TSKFYGGWH | M2;M14 | LATNNLVVMA | M36;M50;M44 |
| 1028 | LKSIAATRGATVVIGT | M17;M72; | LLVYAADPAMHAAS | M12;M24;M57;M50;M33; |
| | SKFYGGWHN | M2;M14 | GNLLLDKRTTC | M8;M46;M39 |
| 1029 | TFEYVSQPFLMDLEGK | M1;M36; | LVYAADPAMHAASG | M12;M24;M57;M50;M33; |
| | QGNFKNLRE | M46;M20 | NLLLDKRTTCF | M8;M46;M39 |
| 1030 | FEYVSQPFLMDLEGKQ | M1;M36; | DTVSALVYDNKLKA | M13;M21;M28;M16;M7; |
| | GNFKNLREF | M46;M20 | HKDKSAQCFKM | M50;M63;M17 |
| 1031 | GDQELIRQGTDYKHW | M64;M69; | VHVVDGCNSSTCMM | M55;M39;M8;M44;M55; |
| | PQIAQFAPSA | M82;M51 | CYKRNRATRVE | M39;M8;M44 |
| 1032 | ETGTLIVNSVLLFLAFV | M50;M73; | LATNNLVVMAYITGG | M51;M42;M9;M12;M51; |
| | VFLLVTLA | M14;M44 | VVQLTSQWLT | M42;M9;M12 |
| 1033 | ETTADIVVFDEISMAT | M33;M7; | TRLQSLENVAFNVVN | M4;M71;M54;M16;M24;M1; |
| | NYDLSVVNA | M48;M55 | KGHFDGQQGE | M27;M15 |
| 1034 | RKRISNCVADYSVLYN | M15;M7; | YGIATVREVLSDREL | M28;M70;M5;M7;M6;M49; |
| | SASFSTFKC | M67;M44 | HLSWEVGKPR | M11;M59 |
| 1035 | MCDIRQLLFVVEVVD | M51;M70; | GIATVREVLSDRELHL | M28;M70;M5;M7;M6;M49; |
| | KYFDCYDGGC | M37;M20 | SWEVGKPRP | M11;M59 |
| 1036 | CDIRQLLFVVEVVDKY | M51;M70; | IATVREVLSDRELHLS | M28;M70;M5;M7;M6;M49; |
| | FDCYDGGCI | M37;M20 | WEVGKPRPP | M11;M59 |
| 1037 | TMCDIRQLLFVVEVVD | M51;M70; | LDDFVEIIKSQDLSVV | M4;M48;M18;M8;M43;M15; |
| | KYFDCYDGG | M37;M20 | SKVVKVTID | M35;M47 |
| 1038 | PDPSKPSKRSFIEDLLF | M73;M63; | PANSTVLSFCAFAVD | M36;M32;M12;M28;M36; |
| | NKVTLADA | M67;M44 | AAKAYKDYLA | M32;M12;M28 |
| 1039 | LIIMRTFKVSIWNLDYI | M1;M5; | CTERLKLFAAETLKA | M4;M64;M21;M40;M48; |
| | INLIIKNL | M44;M20 | TEETFKLSYG | M63;M22;M23 |
| 1040 | IIMRTFKVSIWNLDYII | M1;M5; | TCTERLKLFAAETLK | M4;M64;M21;M40;M48; |
| | NLIIKNLS | M44;M20 | ATEETFKLSY | M63;M22;M23 |
| 1041 | LFIRQEEVQELYSPIFLI | M4;M1; | VGGNYNYLYRLFRKS | M4;M71;M16;M1;M37;M18; |
| | VAAIVFI | M11;M54 | NLKPFERDIS | M17;M15 |
| 1042 | KLFIRQEEVQELYSPIF | M4;M1; | SKVGGNYNYLYRLFR | M4;M71;M16;M1;M37;M18; |
| | LIVAAIVF | M11;M54 | KSNLKPFERD | M17;M15 |
| 1043 | EITVATSRTLSYYKLG | M25;M63; | DSKVGGNYNYLYRLF | M4;M71;M16;M1;M37;M18; |
| | ASQRVAGDS | M19;M70 | RKSNLKPFER | M17;M15 |
| 1044 | ITVATSRTLSYYKLGA | M25;M63; | LDSKVGGNYNYLYRL | M4;M71;M16;M1;M37;M18; |
| | SQRVAGDSG | M19;M70 | FRKSNLKPFE | M17;M15 |
| 1045 | VATSRTLSYYKLGASQ | M25;M63; | KVGGNYNYLYRLFR | M4;M71;M16;M1;M37;M18; |
| | RVAGDSGFA | M19;M70 | KSNLKPFERDI | M17;M15 |
| 1046 | TVATSRTLSYYKLGAS | M25;M63; | NLDSKVGGNYNYLY | M4;M71;M16;M1;M37;M18; |
| | QRVAGDSGF | M19;M70 | RLFRKSNLKPF | M17;M15 |
| 1047 | DTWFSQRGGSYTNDK | M36;M6; | PLYAFASEAARVVRSI | M4;M14;M6;M59;M4;M14; |
| | ACPLIAAVIT | M36;M6 | FSRTLETAQ | M6;M59 |
| 1048 | FSQRGGSYTNDKACPL | M36;M6; | LGSLAATVRLQAGNA | M55;M51;M17;M10;M55; |
| | IAAVITREV | M36;M6 | TEVPANSTVL | M51;M17;M10 |
| 1049 | WFSQRGGSYTNDKAC | M36;M6; | IQRKYKGIKIQEGVVD | M51;M5;M67;M40;M51;M5; |
| | PLIAAVITRE | M36;M6 | YGARFYFYT | M67;M40 |
| 1050 | TWFSQRGGSYTNDKA | M36;M6; | KSVYYTSNPTTFHLD | M50;M7;M48;M45;M50;M7; |
| | CPLIAAVITR | M36;M6 | GEVITFDNLK | M48;M45 |
| 1051 | FDTWFSQRGGSYTND | M36;M6; | VRTIKVFTTVDNINLH | M51;M43;M26;M67;M51; |
| | KACPLIAAVI | M36;M6 | TQVVDMSMT | M43;M26;M67 |
| 1052 | IFLWLLWPVTLACFVL | M33;M63; | DQVILLNKHIDAYKTF | M73;M14;M53;M44;M41; |
| | AAVYRINWI | M14;M70 | PPTEPKKDK | M35;M52;M47 |
| 1053 | KLIFLWLLWPVTLACF | M33;M63; | NALLSTDGNKIADKY | M54;M10;M60;M57;M27; |
| | VLAAVYRIN | M14;M70 | VRNLQHRLYE | M8;M44;M20 |
| 1054 | LIFLWLLWPVTLACFV | M33;M63; | ARDGCVPLNIIPLTTA | M24;M22;M19;M14;M24; |
| | LAAVYRINW | M14;M70 | AKLMVVIPD | M22;M19;M14 |
| 1055 | QGNFKNLREFVFKNID | M6;M19; | PLTTAAKLMVVIPDY | M57;M51;M82;M23;M57; |
| | GYFKIYSKH | M54;M59 | NTYKNTCDGT | M51;M82;M23 |
| 1056 | VRITGLYPTLNISDEFS | M34;M73; | TFLKKDAPYIVGDVV | M7;M30;M11;M14;M7;M30; |
| | SNVANYQK | M6;M44 | QEGVLTAVVI | M11;M14 |
| 1057 | SLQTYVTQQLIRAAEI | M64;M21; | ITFLKKDAPYIVGDVV | M7;M30;M11;M14;M7;M30; |
| | RASANLAAT | M12;M14 | QEGVLTAVV | M11;M14 |
| 1058 | DLFMRIFTIGTVTLKQ | M5;M72; | LKKDAPYIVGDVVQE | M7;M30;M67;M14;M7;M30; |
| | GEIKDATPS | M2;M6 | GVLTAVVIPT | M67;M14 |
| 1059 | MDLFMRIFTIGTVTLK | M5;M72; | KKDAPYIVGDVVQEG | M7;M30;M67;M14;M7;M30; |
| | QGEIKDATP | M2;M6 | VLTAVVIPTK | M67;M14 |
| 1060 | LYSPIFLIVAAIVFITLC | M25;M12; | LTENLLLYIDINGNLH | M7;M36;M11;M70;M7;M36; |
| | FTLKRKT | M14;M37 | PDSATLVSD | M11;M70 |
| 1061 | YSPIFLIVAAIVFITLCF | M25;M12; | FLTENLLLYIDINGNL | M7;M36;M11;M70;M7;M36; |
| | TLKRKTE | M14;M37 | HPDSATLVS | M11;M70 |
| 1062 | YPTLNISDEFSSNVAN | M18;M73; | GKPVPYCYDTNVLEG | M46;M29;M12;M14;M46; |
| | YQKVGMQKY | M6;M44 | SVAYESLRPD | M29;M12;M14 |
| 1063 | LNISDEFSSNVANYQK | M18;M73; | LNSIIKTIQPRVEKKKL | M29;M72;M6;M28;M29; |
| | VGMQKYSTL | M6;M44 | DGFMGRIR | M72;M6;M28 |
| 1064 | ITGLYPTLNISDEFSSN | M18;M73; | LGRCDIKDLPKEITVA | M64;M6;M48;M33;M49; |
| | VANYQKVG | M6;M44 | TSRTLSYYK | M46;M22;M23 |
| 1065 | ISDEFSSNVANYQKVG | M18;M73; | KRGDKSVYYTSNPTT | M50;M22;M45;M44;M50; |
| | MQKYSTLQG | M6;M44 | FHLDGEVITF | M22;M45;M44 |
| 1066 | TLNISDEFSSNVANYQ | M18;M73; | LKRGDKSVYYTSNPT | M50;M22;M45;M44;M50; |
| | KVGMQKYST | M6;M44 | TFHLDGEVIT | M22;M45;M44 |
| 1067 | LYPTLNISDEFSSNVAN | M18;M73; | GSFCTQLNRALTGIAV | M28;M14;M70;M41;M67; |
| | YQKVGMQK | M6;M44 | EQDKNTQEV | M27;M43;M20 |
| 1068 | PTLNISDEFSSNVANY | M18;M73; | QYGSFCTQLNRALTGI | M28;M14;M70;M41;M67; |
| | QKVGMQKYS | M6;M44 | AVEQDKNTQ | M27;M43;M20 |
| 1069 | TGLYPTLNISDEFSSNV | M18;M73; | YGSFCTQLNRALTGIA | M28;M14;M70;M41;M67; |
| | ANYQKVGM | M6;M44 | VEQDKNTQE | M27;M43;M20 |
| 1070 | GLYPTLNISDEFSSNVA | M18;M73; | TYNLWNTFTRLQSLE | M54;M16;M14;M25;M34; |
| | NYQKVGMQ | M6;M44 | NVAFNVVNKG | M60;M42;M39 |
| 1071 | NISDEFSSNVANYQKV | M18;M73; | VTSNYSGVVTTVMFL | M38;M29;M12;M14;M38; |
| | GMQKYSTLQ | M6;M44 | ARGIVFMCVE | M29;M12;M14 |
| 1072 | FIRQEEVQELYSPIFLIV | M4;M1; | ANKDGIIWVATEGAL | M64;M40;M34;M30;M6; |
| | AAIVFIT | M54;M25 | NTPKDHIGTR | M72;M49;M22 |
| 1073 | RQEEVQELYSPIFLIVA | M4;M1; | HFSMMILSDDAVVCF | M13;M21;M7;M53;M48; |
| | AIVFITLC | M54;M25 | NSTYASQGLV | M49;M26;M22 |
| 1074 | IRQEEVQELYSPIFLIV | M4;M1; | FSMMILSDDAVVCFN | M13;M21;M7;M53;M48; |
| | AAIVFITL | M54;M25 | STYASQGLVA | M49;M26;M22 |
| 1075 | QEEVQELYSPIFLIVAA | M4;M1; | TREEAIRHVRAWIGF | M64;M32;M14;M48;M57; |
| | IVFITLCF | M54;M25 | DVEGCHATRE | M69;M46;M20 |
| 1076 | RFPNITNLCPFGEVFNA | M45;M17; | QTSNFRVQPTESIVRF | M21;M31;M34;M30;M48; |
| | TRFASVYA | M28;M66 | PNITNLCPF | M50;M41;M39 |
| 1077 | VCEFQFCNDPFLGVYY | M4;M1; | TAAKLMVVIPDYNTY | M51;M41;M82;M23;M51; |
| | HKNNKSWME | M22;M37 | KNTCDGTTFT | M41;M82;M23 |
| 1078 | CEFQFCNDPFLGVYYH | M4;M1; | RVCTNYMPYFFTLLL | M4;M25;M29;M19;M4;M25; |
| | KNNKSWMES | M22;M37 | QLCTFTRSTN | M29;M19 |
| 1079 | IPLMYKGLPWNVVRIK | M57;M1; | NRVCTNYMPYFFTLL | M4;M25;M29;M19;M4;M25; |
| | IVQMLSDTL | M12;M20 | LQLCTFTRST | M29;M19 |
| 1080 | IEDLLFNKVTLADAGFI | M48;M73; | VCTNYMPYFFTLLLQ | M4;M25;M29;M19;M4;M25; |
| | KQYGDCLG | M47;M44 | LCTFTRSTNS | M29;M19 |
| 1081 | EPMLQSADAQSFLNR | M15;M47; | SNYSGVVTTVMFLAR | M29;M12;M14;M20;M29; |
| | VCGVSAARLT | M12;M45 | GIVFMCVEYC | M12;M14;M20 |
| 1082 | CSCDQLREPMLQSAD | M81;M33; | NYSGVVTTVMFLARG | M29;M12;M14;M20;M29; |
| | AQSFLNGFAV | M12;M45 | IVFMCVEYCP | M12;M14;M20 |
| 1083 | YGCSCDQLREPMLQS | M81;M33; | LGSLIYSTAALGVLMS | M29;M73;M6;M44;M29; |
| | ADAQSFLNGF | M12;M45 | NLGMPSYCT | M73;M6;M44 |
| 1084 | GCSCDQLREPMLQSA | M81;M33; | VCLGSLIYSTAALGVL | M29;M73;M6;M44;M29; |
| | DAQSFLNGFA | M12;M45 | MSNLGMPSY | M73;M6;M44 |
| 1085 | PIGALDISASIVAGGIV | M12;M14; | CLGSLIYSTAALGVL | M29;M73;M6;M44;M29; |
| | AIVVTCLA | M12;M14 | MSNLGMPSYC | M73;M6;M44 |
| 1086 | QPIGALDISASIVAGGI | M12;M14; | QEKNFTTAPAICHDG | M56;M24;M7;M45;M79; |
| | VAIVVTCL | M12;M14 | KAHFPREGVF | M33;M35;M82 |
| 1087 | IGALDISASIVAGGIVAI | M12;M14; | AQEKNFTTAPAICHD | M56;M24;M7;M45;M79; |
| | VVTCLAY | M12;M14 | GKAHFPREGV | M33;M35;M82 |
| 1088 | GALDISASIVAGGIVAI | M12;M14; | GAIKLDDKDPNFKDQ | M73;M10;M7;M53;M44; |
| | VVTCLAYY | M12;M14 | VILLNKHIDA | M35;M52;M47 |
| 1089 | ALDPLSETKCTLKSFT | M75;M21; | VESCGNFKVTKGKAK | M25;M41;M19;M8;M25; |
| | VEKGIYQTS | M63;M14 | KGAWNIGEQK | M41;M19;M8 |
| 1090 | DYVYLPYPDPSRILGA | M57;M63; | QIVESCGNFKVTKGK | M25;M41;M19;M8;M25; |
| | GCFVDDIVK | M12;M20 | AKKGAWNIGE | M41;M19;M8 |
| 1091 | YLPYPDPSRILGAGCF | M57;M63; | IVESCGNFKVTKGKA | M25;M41;M19;M8;M25; |
| | VDDIVKTDG | M12;M20 | KKGAWNIGEQ | M41;M19;M8 |
| 1092 | VYLPYPDPSRILGAGC | M57;M63; | PTDQSSYIVDSVTVKN | M50;M18;M12;M36;M50; |
| | FVDDIVKTD | M12;M20 | GSIHLYFDK | M18;M12;M36 |
| 1093 | LPYPDPSRILGAGCFV | M57;M63; | EMLAKALRKVPTDN | M48;M29;M14;M49;M48; |
| | DDIVKTDGT | M12;M20 | YITTYPGQGLN | M29;M14;M49 |
| 1094 | YVYLPYPDPSRILGAG | M57;M63; | TLADAGFIKQYGDCL | M21;M70;M48;M57;M50; |
| | CFVDDIVKT | M12;M20 | GDIAARDLIC | M63;M41;M27 |
| 1095 | PYPDPSRILGAGCFVD | M57;M63; | LADAGFIKQYGDCLG | M21;M70;M48;M57;M50; |
| | DIVKTDGTL | M12;M20 | DIAARDLICA | M63;M41;M27 |
| 1096 | MLKTVYSDVENPHLM | M45;M43; | ADAGFIKQYGDCLGD | M21;M70;M48;M57;M50; |
| | GWDYPKCDRA | M6;M59 | IAARDLICAQ | M63;M41;M27 |
| 1097 | NMLKTVYSDVENPHL | M45;M43; | TYVPAQEKNFTTAPAI | M78;M65;M7;M29;M2;M79; |
| | MGWDYPKCDR | M6;M59 | CHDGKAHFP | M33;M82 |
| 1098 | LKTVYSDVENPHLMG | M45;M43; | VTYVPAQEKNFTTAP | M78;M65;M7;M29;M2;M79; |
| | WDYPKCDRAM | M6;M59 | AICHDGKAHF | M33;M82 |
| 1099 | KRTTCFSVAALTNNV | M1;M22; | HVTYVPAQEKNFTTA | M78;M65;M7;M29;M2;M79; |
| | AFQTVKPGNF | M6;M73 | PAICHDGKAH | M33;M82 |
| 1100 | LYENQKLIANQFNSAI | M4;M1; | SRELKVTFFPDLNGD | M60;M21;M63;M36;M60; |
| | GKIQDSLSS | M33;M37 | VVAIDYKHYT | M21;M63;M36 |
| 1101 | ENQKLIANQFNSAIGKI | M4;M1; | ASRELKVTFFPDLNG | M60;M21;M63;M36;M60; |
| | QDSLSSTA | M33;M37 | DVVAIDYKHY | M21;M63;M36 |
| 1102 | TQNVLYENQKLIANQF | M4;M1; | NAQALNTLVKQLSSN | M58;M16;M53;M45;M17; |
| | NSAIGKIQD | M33;M37 | FGAISSVLND | M15;M46;M22 |
| 1103 | YENQKLIANQFNSAIG | M4;M1; | QNAQALNTLVKQLSS | M58;M16;M53;M45;M17; |
| | KIQDSLSST | M33;M37 | NFGAISSVLN | M15;M46;M22 |
| 1104 | QNVLYENQKLIANQF | M4;M1; | NPTDQSSYIVDSVTVK | M50;M18;M63;M36;M50; |
| | NSAIGKIQDS | M33;M37 | NGSIHLYFD | M18;M63;M36 |
| 1105 | NVLYENQKLIANQFNS | M4;M1; | INPTDQSSYIVDSVTV | M50;M18;M63;M36;M50; |
| | AIGKIQDSL | M33;M37 | KNGSIHLYF | M18;M63;M36 |
| 1106 | VLYENQKLIANQFNSA | M4;M1; | PINPTDQSSYIVDSVT | M50;M18;M63;M36;M50; |
| | IGKIQDSLS | M33;M37 | VKNGSIHLY | M18;M63;M36 |
| 1107 | TEETFKLSYGIATVRE | M33;M12; | KRPINPTDQSSYIVDS | M50;M18;M63;M36;M50; |
| | VLSDRELHL | M6;M59 | VTVKNGSIH | M18;M63;M36 |
| 1108 | SSNFGAISSVLNDILSR | M50;M48; | RPINPTDQSSYIVDSV | M50;M18;M63;M36;M50; |
| | LDKVEAEV | M6;M17 | TVKNGSIHL | M18;M63;M36 |
| 1109 | HLGRCDIKDLPKEITV | M33;M6; | AALGVLMSNLGMPS | M18;M73;M45;M44;M18; |
| | ATSRTLSYY | M48;M49 | YCTGYREGYLN | M73;M45;M44 |
| 1110 | WYFYYLGTGPEAGLP | M32;M12; | LGVLMSNLGMPSYCT | M18;M73;M45;M44;M18; |
| | YGANKDGIIW | M6;M31 | GYREGYLNST | M73;M45;M44 |
| 1111 | PRWYFYYLGTGPEAG | M32;M12; | ALGVLMSNLGMPSYC | M18;M73;M45;M44;M18; |
| | LPYGANKDGI | M6;M31 | TGYREGYLNS | M73;M45;M44 |
| 1112 | RWYFYYLGTGPEAGL | M32;M12; | IRKSNHNFLVQAGNV | M58;M12;M14;M70;M58; |
| | PYGANKDGII | M6;M31 | QLRVIGHSMQ | M12;M14;M70 |
| 1113 | ISFMLWCKDGHVETF | M71;M36; | YTTTIKPVTYKLDGV | M36;M6;M59;M20;M36;M6; |
| | YPKLQSSQAW | M67;M47 | VCTEIDPKLD | M59;M20 |
| 1114 | EISFMLWCKDGHVETF | M71;M36; | SYTTTIKPVTYKLDGV | M36;M6;M59;M20;M36;M6; |
| | YPKLQSSQA | M67;M47 | VCTEIDPKL | M59;M20 |
| 1115 | DKVFRSSVLHSTQDLF | M72;M29; | TTTIKPVTYKLDGVV | M36;M6;M59;M20;M36;M6; |
| | LPFFSNVTW | M67;M6 | CTEIDPKLDN | M59;M20 |
| 1116 | VRIKIVQMLSDTLKNL | M57;M46; | TFSSTFNVPMEKLKTL | M29;M22;M6;M28;M29; |
| | SDRVVFVLW | M61;M36 | VATAEAELA | M22;M6;M28 |
| 1117 | VTQNVLYENQKLIAN | M4;M1; | GVSFSTFEEAALCTFL | M13;M6;M44;M49;M13;M6; |
| | QFNSAIGKIQ | M53;M37 | LNKEMYLKL | M44;M49 |
| 1118 | GVTQNVLYENQKLIA | M4;M1; | YQCAMRPNFTIKGSF | M52;M47;M12;M14;M52; |
| | NQFNSAIGKI | M53;M37 | LNGSCGSVGF | M47;M12;M14 |
| 1119 | IGVTQNVLYENQKLIA | M4;M1; | VYQCAMRPNFTIKGS | M52;M47;M12;M14;M52; |
| | NQFNSAIGK | M53;M37 | FLNGSCGSVG | M47;M12;M14 |
| 1120 | REVLSDRELHLSWEV | M5;M12; | CAMRPNFTIKGSFLN | M52;M47;M12;M14;M52; |
| | GKPRPPLNRN | M28;M70 | GSCGSVGFNI | M47;M12;M14 |
| 1121 | EVLSDRELHLSWEVG | M5;M12; | AMRPNFTIKGSFLNGS | M52;M47;M12;M14;M52; |
| | KPRPPLNRNY | M28;M70 | CGSVGFNID | M47;M12;M14 |
| 1122 | VLSDRELHLSWEVGK | M5;M12; | QCAMRPNFTIKGSFL | M52;M47;M12;M14;M52; |
| | PRPPLNRNYV | M28;M70 | NGSCGSVGFN | M47;M12;M14 |
| 1123 | CVPQADVEWKFYDAQ | M41;M6; | MRPNFTIKGSFLNGSC | M52;M47;M12;M14;M52; |
| | PCSDKAYKIE | M59;M8 | GSVGFNIDY | M47;M12;M14 |
| 1124 | VPQADVEWKFYDAQP | M41;M6; | GPTYLDGADVTKIKP | M55;M53;M12;M48;M55; |
| | CSDKAYKIEE | M59;M8 | HNSHEGKTFY | M53;M12;M48 |
| 1125 | DGYFKIYSKHTPINLV | M64;M36; | DASGKPVPYCYDTNV | M35;M30;M29;M36;M35; |
| | RDLPQGFSA | M40;M71 | LEGSVAYESL | M30;M29;M36 |
| 1126 | QYVFCTVNALPETTAD | M38;M6; | FKDASGKPVPYCYDT | M35;M30;M29;M36;M35; |
| | IVVFDEISM | M28;M49 | NVLEGSVAYE | M30;M29;M36 |
| 1127 | KEITVATSRTLSYYKL | M49;M63; | IFKDASGKPVPYCYD | M35;M30;M29;M36;M35; |
| | GASQRVAGD | M48;M70 | TNVLEGSVAY | M30;M29;M36 |
| 1128 | TERLKLFAAETLKATE | M4;M63; | KDASGKPVPYCYDTN | M35;M30;M29;M36;M35; |
| | ETFKLSYGI | M40;M64 | VLEGSVAYES | M30;M29;M36 |
| 1129 | SPRRARSVASQSIIAYT | M5;M48; | LQKEKVNINIVGDFKL | M7;M27;M44;M35;M7;M27; |
| | MSLGAENS | M6;M59 | NEEIAIILA | M44;M35 |
| 1130 | NSPRRARSVASQSIIAY | M5;M48; | ILQKEKVNINIVGDFK | M7;M27;M44;M35;M7;M27; |
| | TMSLGAEN | M6;M59 | LNEEIAIIL | M44;M35 |
| 1131 | FSLWVYKQFDTYNLW | M71;M25; | DTSLSGFKLKDCVMY | M15;M75;M12;M14;M15; |
| | NTFTRLQSLE | M41;M19 | ASAVVLLILM | M75;M12;M14 |
| 1132 | SFMLWCKDGHVETFY | M15;M36; | VDTSLSGFKLKDCVM | M15;M75;M12;M14;M15; |
| | PKLQSSQAWQ | M67;M47 | YASAVVLLIL | M75;M12;M14 |
| 1133 | TQTNSPRRARSVASQS | M6;M5;M2; | NTFSSTFNVPMEKLK | M29;M22;M28;M8;M29; |
| | IIAYTMSLG | M59 | TLVATAEAEL | M22;M28;M8 |
| 1134 | QTQTNSPRRARSVASQ | M6;M5;M2; | LLSAGIFGADPIHSLR | M68;M36;M28;M31;M68; |
| | SIIAYTMSL | M59 | VCVDTVRTN | M36;M28;M31 |
| 1135 | QTNSPRRARSVASQSII | M6;M5;M2; | LSAGIFGADPIHSLRV | M68;M36;M28;M31;M68; |
| | AYTMSLGA | M59 | CVDTVRTNV | M36;M28;M31 |
| 1136 | CCNIVNVSLVKPSFYV | M33;M12; | AGIFGADPIHSLRVCV | M68;M36;M28;M31;M68; |
| | YSRVKNLNS | M16;M71 | DTVRTNVYL | M36;M28;M31 |
| 1137 | NIVNVSLVKPSFYVYS | M33;M12; | SAGIFGADPIHSLRVC | M68;M36;M28;M31;M68; |
| | RVKNLNSSR | M16;M71 | VDTVRTNVY | M36;M28;M31 |
| 1138 | CNIVNVSLVKPSFYVY | M33;M12; | DISASIVAGGIVAIVVT | M21;M12;M6;M22;M21; |
| | SRVKNLNSS | M16;M71 | CLAYYFMR | M12;M6;M22 |
| 1139 | RRNVATLQAENVTGL | M36;M29; | APLLSAGIFGADPIHS | M36;M31;M28;M20;M36; |
| | FKDCSKVITG | M38;M47 | LRVCVDTVR | M31;M28;M20 |
| 1140 | RNVATLQAENVTGLF | M36;M29; | PLLSAGIFGADPIHSLR | M36;M31;M28;M20;M36; |
| | KDCSKVITGL | M38;M47 | VCVDTVRT | M31;M28;M20 |
| 1141 | DRYPANSIVCRFDTRV | M5;M9;M2; | DFGDFIQTTPGSGVPV | M40;M9;M58;M16;M34;M8; |
| | LSNLNLPGC | M48 | VDSYYSLLM | M15;M11 |
| 1142 | RYPANSIVCRFDTRVL | M5;M9;M2; | YDFGDFIQTTPGSGVP | M40;M9;M58;M16;M34;M8; |
| | SNLNLPGCD | M48 | VVDSYYSLL | M15;M11 |
| 1143 | NYGDSATLPKGIMMN | M73;M11; | NWYDFGDFIQTTPGS | M40;M9;M58;M16;M34;M8; |
| | VAKYTQLCQY | M58;M44 | GVPVVDSYYS | M15;M11 |
| 1144 | YGDSATLPKGIMMNV | M73;M11; | GNWYDFGDFIQTTPG | M40;M9;M58;M16;M34;M8; |
| | AKYTQLCQYL | M58;M44 | SGVPVVDSYY | M15;M11 |
| 1145 | GDSATLPKGIMMNVA | M73;M11; | WYDFGDFIQTTPGSG | M40;M9;M58;M16;M34;M8; |
| | KYTQLCQYLN | M58;M44 | VPVVDSYYSL | M15;M11 |
| 1146 | CYMHHMELPTGVHAG | M6;M28; | KEKVNINIVGDFKLNE | M7;M73;M44;M35;M7;M73; |
| | TDLEGNFYGP | M6;M28 | EIAIILASF | M44;M35 |
| 1147 | MHHMELPTGVHAGTD | M6;M28; | KVNINIVGDFKLNEEI | M7;M73;M44;M35;M7;M73; |
| | LEGNFYGPFV | M6;M28 | AIILASFSA | M44;M35 |
| 1148 | YMHHMELPTGVHAGT | M6;M28; | EKVNINIVGDFKLNEE | M7;M73;M44;M35;M7;M73; |
| | DLEGNFYGPF | M6;M28 | LAIILASFS | M44;M35 |
| 1149 | FCYMHHMELPTGVHA | M6;M28; | KHTTCCSLSHRFYRL | M5;M60;M30;M2;M79;M43; |
| | GTDLEGNFYG | M6;M28 | ANECAQVLSE | M42;M39 |
| 1150 | SFCYMHHMELPTGVH | M6;M28; | DGVVQQLPETYFTQS | M71;M32;M16;M31;M45; |
| | AGTDLEGNFY | M6;M28 | RNLQEFKPRS | M33;M35;M47 |
| 1151 | HHMELPTGVHAGTDL | M6;M28; | IDITFLKKDAPYIVGD | M57;M30;M11;M14;M57; |
| | EGNFYGPFVD | M6;M28 | VVQEGVLTA | M30;M11;M14 |
| 1152 | APGTAVLRQWLPTGT | M33;M30; | DIDITFLKKDAPYIVG | M57;M30;M11;M14;M57; |
| | LLVDSDLNDF | M8;M70 | DVVQEGVLT | M30;M11;M14 |
| 1153 | VAPGTAVLRQWLPTG | M33;M30; | SKIITLKKRWQLALSK | M11;M55;M74;M33;M27; |
| | TLLVDSDLND | M8;M70 | GVHFVCNLL | M8;M42;M82 |
| 1154 | GVAPGTAVLRQWLPT | M33;M30; | DDFTGCVIAWNSNNL | M56;M32;M12;M48;M50; |
| | GTLLVDSDLN | M8;M70 | DSKVGGNYNY | M68;M49;M26 |
| 1155 | GTAVLRQWLPTGTLL | M33;M30; | DFTGCVIAWNSNNLD | M56;M32;M12;M48;M50; |
| | VDSDLNDFVS | M8;M70 | SKVGGNYNYL | M68;M49;M26 |
| 1156 | PGTAVLRQWLPTGTLL | M33;M30; | LSVCLGSLIYSTAALG | M29;M73;M6;M8;M29;M73; |
| | VDSDLNDFV | M8;M70 | VLMSNLGMP | M6;M8 |
| 1157 | ALVYDNKLKAHKDKS | M13;M21; | LLLSVCLGSLIYSTAA | M29;M73;M6;M8;M29;M73; |
| | AQCFKMFYKG | M63;M28 | LGVLMSNLG | M6;M8 |
| 1158 | VSALVYDNKLKAHKD | M13;M21; | LLSVCLGSLIYSTAAL | M29;M73;M6;M8;M29;M73; |
| | KSAQCFKMFY | M63;M28 | GVLMSNLGM | M6;M8 |
| 1159 | SALVYDNKLKAHKDK | M13;M21; | SVCLGSLIYSTAALGV | M29;M73;M6;M8;M29;M73; |
| | SAQCFKMFYK | M63;M28 | LMSNLGMPS | M6;M8 |
| 1160 | LVYDNKLKAHKDKSA | M13;M21; | FLLLSVCLGSLIYSTA | M29;M73;M6;M8;M29;M73; |
| | QCFKMFYKGV | M63;M28 | ALGVLMSNL | M6;M8 |
| 1161 | GNFKNLREFVFKNIDG | M71;M19; | WFLLLSVCLGSLIYST | M29;M73;M6;M8;M29;M73; |
| | YFKIYSKHT | M54;M59 | AALGVLMSN | M6;M8 |
| 1162 | ECIKDLLARAGKASCT | M12;M28; | GMPSYCTGYREGYLN | M18;M49;M45;M8;M18; |
| | LSEQLDFID | M12;M28 | STNVTIATYC | M49;M45;M8 |
| 1163 | PLECIKDLLARAGKAS | M12;M28; | VAIHADQLTPTWRVY | M58;M24;M16;M5;M6;M41; |
| | CTLSEQLDF | M12;M28 | STGSNVFQTR | M17;M82 |
| 1164 | LECIKDLLARAGKASC | M12;M28; | AIHADQLTPTWRVYS | M58;M24;M16;M5;M6;M41; |
| | TLSEQLDFI | M12;M28 | TGSNVFQTRA | M17;M82 |
| 1165 | YPLECIKDLLARAGKA | M12;M28; | PVAIHADQLTPTWRV | M58;M24;M16;M5;M6;M41; |
| | SCTLSEQLD | M12;M28 | YSTGSNVFQT | M17;M82 |
| 1166 | IKDLLARAGKASCTLS | M12;M28; | QGMDNLACEDLKPVS | M1;M49;M45;M37;M1;M49; |
| | EQLDFIDTK | M12;M28 | EEVVENPTIQ | M45;M37 |
| 1167 | GYPLECIKDLLARAGK | M12;M28; | AYVNTFSSTFNVPME | M22;M28;M8;M70;M22; |
| | ASCTLSEQL | M12;M28 | KLKTLVATAE | M28;M8;M70 |
| 1168 | CIKDLLARAGKASCTL | M12;M28; | YVNTFSSTFNVPMEK | M22;M28;M8;M70;M22; |
| | SEQLDFIDT | M12;M28 | LKTLVATAEA | M28;M8;M70 |
| 1169 | VTGLFKDCSKVITGLH | M46;M34; | STSGRWVLNNDYYRS | M33;M34;M39;M44;M33; |
| | PTQAPTHLS | M47;M36 | LPGVFCGVDA | M34;M39;M44 |
| 1170 | KRSFIEDLLFNKVTLA | M11;M73; | DAAMQRKLEKMADQ | M57;M63;M82;M2;M57; |
| | DAGFIKQYG | M47;M44 | AMTQMYKQARS | M63;M82;M2 |
| 1171 | SKRSFIEDLLFNKVTLA | M11;M73; | EFDRDAAMQRKLEK | M57;M63;M82;M2;M57; |
| | DAGFIKQY | M47;M44 | MADQAMTQMYK | M63;M82;M2 |
| 1172 | SFIEDLLFNKVTLADA | M11;M73; | FDRDAAMQRKLEKM | M57;M63;M82;M2;M57; |
| | GFIKQYGDC | M47;M44 | ADQAMTQMYKQ | M63;M82;M2 |
| 1173 | RSFIEDLLFNKVTLAD | M11;M73; | DRDAAMQRKLEKMA | M57;M63;M82;M2;M57; |
| | AGFIKQYGD | M47;M44 | DQAMTQMYKQA | M63;M82;M2 |
| 1174 | SNLKPFERDISTEIYQA | M46;M53; | RDAAMQRKLEKMAD | M57;M63;M82;M2;M57; |
| | GSTPCNGV | M17;M44 | QAMTQMYKQAR | M63;M82;M2 |
| 1175 | ISFPLCANGQVFGLYK | M41;M73; | TVKNGSIHLYFDKAG | M7;M21;M48;M49;M7;M21; |
| | NTCVGSDNV | M28;M48 | QKTYERHSLS | M48;M49 |
| 1176 | CANGQVFGLYKNTCV | M41;M73; | VKNGSIHLYFDKAGQ | M7;M21;M48;M49;M7;M21; |
| | GSDNVTDFNA | M28;M48 | KTYERHSLSH | M48;M49 |
| 1177 | SFPLCANGQVFGLYKN | M41;M73; | KNGSIHLYFDKAGQK | M7;M21;M48;M49;M7;M21; |
| | TCVGSDNVT | M28;M48 | TYERHSLSHF | M48;M49 |
| 1178 | FPLCANGQVFGLYKN | M41;M73; | SVTVKNGSIHLYFDK | M7;M21;M48;M49;M7;M21; |
| | TCVGSDNVTD | M28;M48 | AGQKTYERHS | M48;M49 |
| 1179 | PLCANGQVFGLYKNT | M41;M73; | VTVKNGSIHLYFDKA | M7;M21;M48;M49;M7;M21; |
| | CVGSDNVTDF | M28;M48 | GQKTYERHSL | M48;M49 |
| 1180 | LCANGQVFGLYKNTC | M41;M73; | IVDSVTVKNGSIHLYF | M21;M12;M48;M49;M21; |
| | VGSDNVTDFN | M28;M48 | DKAGQKTYE | M12;M48;M49 |
| 1181 | FLGIITTVAAFHQECSL | M30;M29; | VDSVTVKNGSIHLYF | M21;M12;M48;M49;M21; |
| | QSCTQHQP | M6;M59 | DKAGQKTYER | M12;M48;M49 |
| 1182 | VFLGIITTVAAFHQECS | M30;M29; | SYIVDSVTVKNGSIHL | M21;M12;M48;M49;M21; |
| | LQSCTQHQ | M6;M59 | YFDKAGQKT | M12;M48;M49 |
| 1183 | YIATNGPLKVGGSCVL | M80;M14; | YIVDSVTVKNGSIHLY | M21;M12;M48;M49;M21; |
| | SGHNLAKHC | M80;M14 | FDKAGQKTY | M12;M48;M49 |
| 1184 | ATNGPLKVGGSCVLS | M80;M14; | TLEETKFLTENLLLYI | M50;M36;M11;M70;M50; |
| | GHNLAKHCLH | M80;M14 | DINGNLHPD | M36;M11;M70 |
| 1185 | IATNGPLKVGGSCVLS | M80;M14; | LEETKFLTENLLLYIDI | M50;M36;M11;M70;M50; |
| | GHNLAKHCL | M80;M14 | NGNLHPDS | M36;M11;M70 |
| 1186 | TNGPLKVGGSCVLSG | M80;M14; | ALCTFLLNKEMYLKL | M42;M39;M43;M44;M42; |
| | HNLAKHCLHV | M80;M14 | RSDVLLPLTQ | M39;M43;M44 |
| 1187 | DYIATNGPLKVGGSCV | M80;M14; | LCTFLLNKEMYLKLR | M42;M39;M43;M44;M42; |
| | LSGHNLAKH | M80;M14 | SDVLLPLTQY | M39;M43;M44 |
| 1188 | NGPLKVGGSCVLSGH | M80;M14; | CTFLLNKEMYLKLRS | M42;M39;M43;M44;M42; |
| | NLAKHCLHVV | M80;M14 | DVLLPLTQYN | M39;M43;M44 |
| 1189 | LESELVIGAVILRGHLR | M55;M53; | ATNNAMQVESDDYIA | M10;M29;M11;M44;M10; |
| | IAGHHLGR | M58;M14 | TNGPLKVGGS | M29;M11;M44 |
| 1190 | PGNFNKDFYDFAVSK | M33;M25; | YGTEDDYQGKPLEFG | M1;M22;M28;M37;M1;M22; |
| | GFFKEGSSVE | M19;M70 | ATSAALQPEE | M28;M37 |
| 1191 | KPGNFNKDFYDFAVS | M33;M25; | GTEDDYQGKPLEFGA | M1;M22;M28;M37;M1;M22; |
| | KGFFKEGSSV | M19;M70 | TSAALQPEEE | M28;M37 |
| 1192 | NKDFYDFAVSKGFFKE | M33;M25; | LDGISQYSLRLIDAM | M57;M60;M54;M36;M57; |
| | GSSVELKHF | M19;M31 | MFTSDLATNN | M60;M54;M36 |
| 1193 | FNKDFYDFAVSKGFFK | M33;M25; | DGISQYSLRLIDAMM | M57;M60;M54;M36;M57; |
| | EGSSVELKH | M19;M31 | FTSDLATNNL | M60;M54;M36 |
| 1194 | NFNKDFYDFAVSKGFF | M33;M25; | VVFNGVSFSTFEEAAL | M4;M13;M6;M49;M4;M13; |
| | KEGSSVELK | M19;M31 | CTFLLNKEM | M6;M49 |
| 1195 | INCQEPKLGSLVVRCS | M29;M82; | RVVFNGVSFSTFEEA | M4;M13;M6;M49;M4;M13; |
| | FYEDFLEYH | M14;M70 | ALCTFLINKE | M6;M49 |
| 1196 | NCQEPKLGSLVVRCSF | M29;M82; | VFNGVSFSTFEEAALC | M4;M13;M6;M49;M4;M13; |
| | YEDFLEYHD | M14;M70 | TFLLNKEMY | M6;M49 |
| 1197 | ANGQVFGLYKNTCVG | M48;M73; | FNGVSFSTFEEAALCT | M4;M13;M6;M49;M4;M13; |
| | SDNVTDENAI | M28;M59 | FLLNKEMYL | M6;M49 |
| 1198 | LPFTINCQEPKLGSLVV | M50;M29; | KRRVVFNGVSFSTFEE | M4;M13;M6;M49;M4;M13; |
| | RCSFYEDF | M82;M14 | AALCTFLLN | M6;M49 |
| 1199 | LACFVLAAVYRINWIT | M50;M33; | RRVVFNGVSFSTFEEA | M4;M13;M6;M49;M4;M13; |
| | GGIAIAMAC | M58;M44 | ALCTFLLNK | M6;M49 |
| 1200 | TLACFVLAAVYRINWI | M50;M33; | VENPDILRVYANLGE | M64;M11;M21;M32;M48; |
| | TGGIAIAMA | M58;M44 | RVRQALLKTV | M41;M49;M22 |
| 1201 | QKRTATKAYNVTQAF | M78;M25; | AYTVELGTEVNEFAC | M14;M34;M39;M66;M14; |
| | GRRGPEQTQG | M19;M70 | VVADAVIKTL | M34;M39;M66 |
| 1202 | RTATKAYNVTQAFGR | M78;M25; | AVKMFDAYVNTFSST | M41;M28;M8;M70;M41; |
| | RGPEQTQGNF | M19;M70 | FNVPMEKLKT | M28;M8;M70 |
| 1203 | RQKRTATKAYNVTQA | M78;M25; | TRFQTLLALHRSYLTP | M21;M24;M70;M55;M5; |
| | FGRRGPEQTQ | M19;M70 | GDSSSGWTA | M48;M9;M23 |
| 1204 | KRTATKAYNVTQAFG | M78;M25; | VFLLVTLAILTALRLC | M54;M16;M60;M25;M41; |
| | RRGPEQTQGN | M19;M70 | AYCCNIVNV | M17;M46;M39 |
| 1205 | TEKWESGVKDCVVLH | M46;M67; | VVFLLVTLAILTALRL | M54;M16;M60;M25;M41; |
| | SYFTSDYYQL | M14;M20 | CAYCCNIVN | M17;M46;M39 |
| 1206 | YTEKWESGVKDCVVL | M46;M67; | FLLVTLAILTALRLCA | M54;M16;M60;M25;M41; |
| | HSYFTSDYYQ | M14;M20 | YCCNIVNVS | M17;M46;M39 |
| 1207 | NDKVAGFAKFLKTNC | M79;M55; | LLVTLAILTALRLCAY | M54;M16;M60;M25;M41; |
| | CRFQEKDEDD | M73;M8 | CCNIVNVSL | M17;M46;M39 |
| 1208 | GVEHVTFFIYNKIVDE | M50;M44; | AFGGCVFSYVGCHNK | M40;M75;M19;M55;M40; |
| | PEEHVQIHT | M47;M49 | CAYWVPRASA | M75;M19;M55 |
| 1209 | TDTGVEHVTFFIYNKI | M50;M44; | VVNVVTTKIALKGGK | M57;M29;M11;M12;M57; |
| | VDEPEEHVQ | M47;M49 | IVNNWLKQLI | M29;M11;M12 |
| 1210 | TGVEHVTFFIYNKIVD | M50;M44; | QVVNVVTTKIALKGG | M57;M29;M11;M12;M57; |
| | EPEEHVQIH | M47;M49 | KIVNNWLKQL | M29;M11;M12 |
| 1211 | DTGVEHVTFFIYNKIV | M50;M44; | IGLALYYPSARIVYTA | M64;M21;M28;M5;M29; |
| | DEPEEHVQI | M47;M49 | CSHAAVDAL | M38;M17;M22 |
| 1212 | VEHVTFFIYNKIVDEPE | M50;M44; | YMLTYNKVENMTPR | M14;M11;M67;M74;M14; |
| | EHVQIHTI | M47;M49 | DLGACIDCSAR | M11;M67;M74 |
| 1213 | TSRYWEPEFYEAMYT | M75;M30; | LKWARFPKSDGTGTI | M38;M6;M59;M28;M38;M6; |
| | PHTVLQAVGA | M39;M16 | YTELEPPCRF | M59;M28 |
| 1214 | DNTSRYWEPEFYEAM | M75;M30; | FPKSDGTGTIYTELEP | M38;M6;M59;M28;M38;M6; |
| | YTPHTVLQAV | M39;M16 | PCRFVTDTP | M59;M28 |
| 1215 | NTSRYWEPEFYEAMY | M75;M30; | WARFPKSDGTGTIYT | M38;M6;M59;M28;M38;M6; |
| | TPHTVLQAVG | M39;M16 | ELEPPCRFVT | M59;M28 |
| 1216 | SRYWEPEFYEAMYTP | M75;M30; | DLKWARFPKSDGTGT | M38;M6;M59;M28;M38;M6; |
| | HTVLQAVGAC | M39;M16 | IYTELEPPCR | M59;M28 |
| 1217 | YWEPEFYEAMYTPHT | M75;M30; | RFPKSDGTGTIYTELE | M38;M6;M59;M28;M38;M6; |
| | VLQAVGACVL | M39;M16 | PPCRFVTDT | M59;M28 |
| 1218 | RYWEPEFYEAMYTPH | M75;M30; | KWARFPKSDGTGTIY | M38;M6;M59;M28;M38;M6; |
| | TVLQAVGACV | M39;M16 | TELEPPCRFV | M59;M28 |
| 1219 | VYRGTTTYKLNVGDY | M42;M34; | QDLKWARFPKSDGTG | M38;M6;M59;M28;M38;M6; |
| | FVLTSHTVMP | M43;M44 | TIYTELEPPC | M59;M28 |
| 1220 | TGKIADYNYKLPDDFT | M13;M29; | ARFPKSDGTGTIYTEL | M38;M6;M59;M28;M38;M6; |
| | GCVIAWNSN | M67;M12 | EPPCRFVTD | M59;M28 |
| 1221 | KFLVFLGIITTVAAFHQ | M29;M22; | WYDFVENPDILRVYA | M64;M21;M32;M48;M41; |
| | ECSLQSCT | M6;M59 | NLGERVRQAL | M49;M46;M22 |
| 1222 | HEVLLAPLLSAGIFGA | M36;M20; | DWYDFVENPDILRVY | M64;M21;M32;M48;M41; |
| | DPIHSLRVC | M36;M20 | ANLGERVRQA | M49;M46;M22 |
| 1223 | FNQHEVLLAPLLSAGI | M36;M20; | ISVTSNYSGVVTTVM | M29;M11;M38;M14;M29; |
| | FGADPIHSL | M36;M20 | FLARGIVFMC | M11;M38;M14 |
| 1224 | LAPLLSAGIFGADPIHS | M36;M20; | IISVTSNYSGVVTTVM | M29;M11;M38;M14;M29; |
| | LRVCVDTV | M36;M20 | FLARGIVFM | M11;M38;M14 |
| 1225 | EVLLAPLLSAGIFGAD | M36;M20; | LIISVTSNYSGVVTTV | M29;M11;M38;M14;M29; |
| | PIHSLRVCV | M36;M20 | MFLARGIVF | M11;M38;M14 |
| 1226 | NQHEVLLAPLLSAGIF | M36;M20; | SAYTVELGTEVNEFA | M43;M34;M39;M14;M43; |
| | GADPIHSLR | M36;M20 | CVVADAVIKT | M34;M39;M14 |
| 1227 | ENSYTTTIKPVTYKLD | M36;M20; | CSAYTVELGTEVNEF | M43;M34;M39;M14;M43; |
| | GVVCTEIDP | M36;M20 | ACVVADAVIK | M34;M39;M14 |
| 1228 | VLLAPLLSAGIFGADPI | M36;M20; | AFASEAARVVRSIFSR | M4;M42;M48;M59;M4;M42; |
| | HSLRVCVD | M36;M20 | TLETAQNSV | M48;M59 |
| 1229 | LLAPLLSAGIFGADPIH | M36;M20; | SNYQHEETIYNLLKD | M40;M30;M41;M42;M27; |
| | SLRVCVDT | M36;M20 | CPAVAKHDFF | M43;M39;M11 |
| 1230 | QHEVLLAPLLSAGIFG | M36;M20; | YQHEETIYNLLKDCP | M40;M30;M41;M42;M27; |
| | ADPIHSLRV | M36;M20 | AVAKHDFFKF | M43;M39;M11 |
| 1231 | CYKRNRATRVECTTIV | M29;M14; | NYQHEETIYNLLKDC | M40;M30;M41;M42;M27; |
| | NGVRRSFYV | M29;M14 | PAVAKHDFFK | M43;M39;M11 |
| 1232 | FRVYSSANNCTFEYVS | M57;M1; | KIKACVEEVTTTLEET | M32;M6;M28;M20;M32;M6; |
| | QPFLMDLEG | M82;M20 | KFLTENLLL | M28;M20 |
| 1233 | KTTLPVNVAFELWAK | M55;M28; | KDWYDFVENPDILRV | M64;M21;M32;M48;M43; |
| | RNIKPVPEVK | M66;M74 | YANLGERVRQ | M49;M46;M22 |
| 1234 | TTLPVNVAFELWAKR | M55;M28; | ITLKKRWQLALSKGV | M11;M74;M33;M27;M8; |
| | NIKPVPEVKI | M66;M74 | HFVCNLLLLF | M42;M82;M23 |
| 1235 | IPARARVECFDKFKVN | M50;M18; | IITLKKRWQLALSKG | M11;M74;M33;M27;M8; |
| | STLEQYVFC | M44;M10 | VHFVCNLLLL | M42;M82;M23 |
| 1236 | SIKNFKSVLYYQNNVF | M41;M22; | FVTQRNFYEPQIITTD | M64;M10;M5;M7;M30;M79; |
| | MSEAKCWTE | M48;M49 | NTFVSGNCD | M9;M23 |
| 1237 | FKSVLYYQNNVFMSE | M41;M22; | GGKGFCKLHNWNCV | M42;M39;M45;M41;M42; |
| | AKCWTETDLT | M48;M49 | NCDTFCAGSTF | M39;M45;M41 |
| 1238 | ASIKNFKSVLYYQNNV | M41;M22; | NGGKGFCKLHNWNC | M42;M39;M45;M41;M42; |
| | FMSEAKCWT | M48;M49 | VNCDTFCAGST | M39;M45;M41 |
| 1239 | KNFKSVLYYQNNVFM | M41;M22; | GLDSLDTYPSLETIQIT | M55;M29;M48;M41;M55; |
| | SEAKCWTETD | M48;M49 | ISSFKWDL | M29;M48;M41 |
| 1240 | NFKSVLYYQNNVFMS | M41;M22; | KAIVSTIQRKYKGIKI | M40;M5;M67;M14;M40;M5; |
| | EAKCWTETDL | M48;M49 | QEGVVDYGA | M67;M14 |
| 1241 | IKNFKSVLYYQNNVF | M41;M22; | AIVSTIQRKYKGIKIQE | M40;M5;M67;M14;M40;M5; |
| | MSEAKCWTET | M48;M49 | GVVDYGAR | M67;M14 |
| 1242 | VASIKNFKSVLYYQNN | M41;M22; | VSTIQRKYKGIKIQEG | M40;M5;M67;M14;M40;M5; |
| | VFMSEAKCW | M48;M49 | VVDYGARFY | M67;M14 |
| 1243 | YEDFQENWNTKHSSG | M74;M44; | IVSTIQRKYKGIKIQEG | M40;M5;M67;M14;M40;M5; |
| | VTRELMRELN | M74;M44 | VVDYGARF | M67;M14 |
| 1244 | EDFQENWNTKHSSGV | M74;M44; | TKAIVSTIQRKYKGIKI | M40;M5;M67;M14;M40;M5; |
| | TRELMRELNG | M74;M44 | QEGVVDYG | M67;M14 |
| 1245 | PYEDFQENWNTKHSS | M74;M44; | LPKGIMMNVAKYTQL | M73;M58;M78;M34;M48; |
| | GVTRELMREL | M74;M44 | CQYLNTLTLA | M41;M49;M11 |
| 1246 | LSVVNARLRAKHYVY | M57;M1; | DLQDLKWARFPKSDG | M4;M38;M28;M59;M4;M38; |
| | IGDPAQLPAP | M31;M37 | TGTIYTELEP | M28;M59 |
| 1247 | DTYACWHHSIGFDYV | M57;M1; | NSIIKTIQPRVEKKKL | M26;M29;M72;M28;M26; |
| | YNPFMIDVQQ | M43;M20 | DGFMGRIRS | M29;M72;M28 |
| 1248 | TYACWHHSIGFDYVY | M57;M1; | NSTYASQGLVASIKNF | M64;M21;M54;M48;M68; |
| | NPFMIDVQQW | M43;M20 | KSVLYYQNN | M49;M22;M23 |
| 1249 | LTNNVAFQTVKPGNF | M25;M29; | TDFVNEFYAYLRKHF | M71;M16;M57;M18;M35; |
| | NKDFYDFAVS | M6;M59 | SMMILSDDAV | M15;M52;M26 |
| 1250 | ALTNNVAFQTVKPGN | M25;M29; | DTDFVNEFYAYLRKH | M71;M16;M57;M18;M35; |
| | FNKDFYDFAV | M6;M59 | FSMMILSDDA | M15;M52;M26 |
| 1251 | VFCTVNALPETTADIV | M38;M55; | IKVTLVFLFVAAIFYLI | M1;M34;M39;M18;M1;M34; |
| | VFDEISMAT | M28;M49 | TPVHVMSK | M39;M18 |
| 1252 | IPTITQMNLKYAISAKN | M35;M82; | LKDCVMYASAVVLLI | M75;M39;M12;M46;M75; |
| | RARTVAGV | M18;M14 | LMTARTVYDD | M39;M12;M46 |
| 1253 | LQSADAQSFLNRVCG | M15;M46; | TPKYKFVRIQPGQTFS | M34;M29;M27;M14;M34; |
| | VSAARLTPCG | M47;M45 | VLACYNGSP | M29;M27;M14 |
| 1254 | MLQSADAQSFLNRVC | M15;M46; | YTDFATSACVLAAEC | M70;M29;M14;M62;M70; |
| | GVSAARLTPC | M47;M45 | TIFKDASGKP | M29;M14;M62 |
| 1255 | SADAQSFLNRVCGVS | M15;M46; | EYTDFATSACVLAAE | M70;M29;M14;M62;M70; |
| | AARLTPCGTG | M47;M45 | CTIFKDASGK | M29;M14;M62 |
| 1256 | QSADAQSFLNRVCGV | M15;M46; | SKLWAQCVQLHNDIL | M36;M32;M68;M58;M36; |
| | SAARLTPCGT | M47;M45 | LAKDTTEAFE | M32;M68;M58 |
| 1257 | PMLQSADAQSFLNRV | M15;M46; | GVHFVCNLLLLFVTV | M21;M6;M37;M50;M41; |
| | CGVSAARLTP | M47;M45 | YSHLLLVAAG | M43;M42;M22 |
| 1258 | TNNVAFQTVKPGNFN | M29;M6; | KLKVDTANPKTPKYK | M77;M29;M34;M14;M77; |
| | KDFYDFAVSK | M59;M70 | FVRIQPGQTF | M29;M34;M14 |
| 1259 | NNVAFQTVKPGNFNK | M29;M6; | KGLNNLNRGMVLGS | M55;M12;M17;M10;M55; |
| | DFYDFAVSKG | M59;M70 | LAATVRLQAGN | M12;M17;M10 |
| 1260 | NVAFQTVKPGNFNKD | M29;M6; | PNASFDNFKFVCDNIK | M64;M25;M19;M23;M64; |
| | FYDFAVSKGF | M59;M70 | FADDLNQLT | M25;M19;M23 |
| 1261 | RNVIPTITQMNLKYAIS | M18;M35; | VVFLHVTYVPAQEKN | M28;M66;M70;M78;M79; |
| | AKNRARTV | M58;M14 | FTTAPAICHD | M33;M26;M82 |
| 1262 | SASAFFGMSRIGMEVT | M5;M28; | GVVFLHVTYVPAQEK | M28;M66;M70;M78;M79; |
| | PSGTWLTYT | M16;M23 | NFTTAPAICH | M33;M26;M82 |
| 1263 | DVEWKFYDAQPCSDK | M29;M6; | VFLHVTYVPAQEKNF | M28;M66;M70;M78;M79; |
| | AYKIEELFYS | M59;M41 | TTAPAICHDG | M33;M26;M82 |
| 1264 | QADVEWKFYDAQPCS | M29;M6; | HGVVFLHVTYVPAQE | M28;M66;M70;M78;M79; |
| | DKAYKIEELF | M59;M41 | KNFTTAPAIC | M33;M26;M82 |
| 1265 | VEWKFYDAQPCSDKA | M29;M6; | DGADVTKIKPHNSHE | M55;M21;M53;M48;M55; |
| | YKIEELFYSY | M59;M41 | GKTFYVLPND | M21;M53;M48 |
| 1266 | ADVEWKFYDAQPCSD | M29;M6; | PTYLDGADVTKIKPH | M55;M21;M53;M48;M55; |
| | KAYKIEELFY | M59;M41 | NSHEGKTFYV | M21;M53;M48 |
| 1267 | FRELGVVHNQDVNLH | M21;M53; | LDGADVTKIKPHNSH | M55;M21;M53;M48;M55; |
| | SSRLSFKELL | M38;M14 | EGKTFYVLPN | M21;M53;M48 |
| 1268 | RELGVVHNQDVNLHS | M21;M53; | YLDGADVTKIKPHNS | M55;M21;M53;M48;M55; |
| | SRLSFKELLV | M38;M14 | HEGKTFYVLP | M21;M53;M48 |
| 1269 | ELGVVHNQDVNLHSS | M21;M53; | TYLDGADVTKIKPHN | M55;M21;M53;M48;M55; |
| | RLSFKELLVY | M38;M14 | SHEGKTFYVL | M21;M53;M48 |
| 1270 | VVHNQDVNLHSSRLSF | M21;M53; | CHIDHPNPKGFCDLK | M42;M39;M63;M43;M42; |
| | KELLVYAAD | M38;M14 | GKYVQIPTTC | M39;M63;M43 |
| 1271 | LGVVHNQDVNLHSSR | M21;M53; | CRCHIDHPNPKGFCD | M42;M39;M63;M43;M42; |
| | LSFKELLVYA | M38;M14 | LKGKYVQIPT | M39;M63;M43 |
| 1272 | GVVHNQDVNLHSSRL | M21;M53; | RCHIDHPNPKGFCDL | M42;M39;M63;M43;M42; |
| | SFKELLVYAA | M38;M14 | KGKYVQIPTT | M39;M63;M43 |
| 1273 | STFEEAALCTFLLNKE | M13;M44; | YLALYNKYKYFSGA | M1;M67;M66;M31;M1;M67; |
| | MYLKLRSDV | M13;M44 | MDTTSYREAAC | M66;M31 |
| 1274 | SFSTFEEAALCTFLLNK | M13;M44; | SEVGPEHSLAEYHNE | M13;M39;M68;M48;M13; |
| | EMYLKLRS | M13;M44 | SGLKTILRKG | M39;M68;M48 |
| 1275 | FSTFEEAALCTFLLNK | M13;M44; | HNSEVGPEHSLAEYH | M13;M39;M68;M48;M13; |
| | EMYLKLRSD | M13;M44 | NESGLKTILR | M39;M68;M48 |
| 1276 | TFEEAALCTFLLNKEM | M13;M44; | NSEVGPEHSLAEYHN | M13;M39;M68;M48;M13; |
| | YLKLRSDVL | M13;M44 | ESGLKTILRK | M39;M68;M48 |
| 1277 | FEEAALCTFLLNKEMY | M13;M44; | NASVVNIQKEIDRLNE | M70;M78;M65;M2;M61; |
| | LKLRSDVLL | M13;M44 | VAKNLNESL | M79;M82;M47 |
| 1278 | AVTANVNALLSTDGN | M57;M9; | ASVVNIQKEIDRLNEV | M70;M78;M65;M2;M61; |
| | KIADKYVRNL | M8;M10 | AKNLNESLI | M79;M82;M47 |
| 1279 | QAVTANVNALLSTDG | M57;M9; | PRVEKKKLDGFMGRI | M26;M29;M6;M58;M26; |
| | NKIADKYVRN | M8;M10 | RSVYPVASPN | M29;M6;M58 |
| 1280 | SAFVNLKQLPFFYYSD | M32;M41; | RVEKKKLDGFMGRIR | M26;M29;M6;M58;M26; |
| | SPCESHGKQ | M54;M45 | SVYPVASPNE | M29;M6;M58 |
| 1281 | TKRNVIPTITQMNLKY | M14;M35; | PFEIKLAKKFDTFNGE | M57;M27;M45;M20;M57; |
| | AISAKNRAR | M58;M59 | CPNFVFPLN | M27;M45;M20 |
| 1282 | YAAVINGDRWFLNRF | M14;M66; | KLAKKFDTFNGECPN | M57;M27;M45;M20;M57; |
| | TTTLNDFNLV | M14;M66 | FVFPLNSIIK | M27;M45;M20 |
| 1283 | AAVINGDRWFLNRFTT | M14;M66; | LAKKFDTFNGECPNF | M57;M27;M45;M20;M57; |
| | TLNDFNLVA | M14;M66 | VFPLNSIIKT | M27;M45;M20 |
| 1284 | AVINGDRWFLNRFTTT | M14;M66; | EIKLAKKFDTFNGECP | M57;M27;M45;M20;M57; |
| | LNDFNLVAM | M14;M66 | NFVFPLNSI | M27;M45;M20 |
| 1285 | WLYAAVINGDRWFLN | M14;M66; | FEIKLAKKFDTFNGEC | M57;M27;M45;M20;M57; |
| | RFTTTLNDFN | M14;M66 | PNFVFPLNS | M27;M45;M20 |
| 1286 | LYAAVINGDRWFLNR | M14;M66; | IKLAKKFDTFNGECPN | M57;M27;M45;M20;M57; |
| | FTTTLNDFNL | M14;M66 | FVFPLNSII | M27;M45;M20 |
| 1287 | ICQAVTANVNALLSTD | M10;M11; | AMQRKLEKMADQA | M55;M82;M2;M74;M55; |
| | GNKIADKYV | M9;M8 | MTQMYKQARSED | M82;M2;M74 |
| 1288 | NICQAVTANVNALLST | M10;M11; | FKEGVEFLRDGWEIV | M4;M22;M74;M49;M4;M22; |
| | DGNKIADKY | M9;M8 | KFISTCACEI | M74;M49 |
| 1289 | VFNICQAVTANVNALL | M10;M11; | TDFATSACVLAAECTI | M33;M70;M14;M62;M33; |
| | STDGNKIAD | M9;M8 | FKDASGKPV | M70;M14;M62 |
| 1290 | FNICQAVTANVNALLS | M10;M11; | TTRQVVNVVTTKIAL | M64;M29;M22;M2;M64; |
| | TDGNKIADK | M9;M8 | KGGKIVNNWL | M29;M22;M2 |
| 1291 | YIINLIIKNLSKSLTENK | M11;M73; | MSFPQSAPHGVVFLH | M64;M21;M28;M66;M70; |
| | YSQLDEE | M28;M17 | VTYVPAQEKN | M53;M26;M9 |
| 1292 | IINLIIKNLSKSLTENKY | M11;M73; | DVGDSAEVAVKMFD | M26;M41;M14;M37;M26; |
| | SQLDEEQ | M28;M17 | AYVNTFSSTFN | M41;M14;M37 |
| 1293 | INLIIKNLSKSLTENKY | M11;M73; | KDEDDNLIDSYFVVK | M71;M58;M16;M54;M18; |
| | SQLDEEQP | M28;M17 | RHTFSNYQHE | M15;M52;M47 |
| 1294 | DYIINLIIKNLSKSLTEN | M11;M73; | EKDEDDNLIDSYFVV | M71;M58;M16;M54;M18; |
| | KYSQLDE | M28;M17 | KRHTFSNYQH | M15;M52;M47 |
| 1295 | DFQVTIAEILLIIMRTF | M1;M5; | QEKDEDDNLIDSYFV | M71;M58;M16;M54;M18; |
| | KVSIWNLD | M61;M37 | VKRHTFSNYQ | M15;M52;M47 |
| 1296 | YFNSVCRLMKTIGPD | M17;M5; | LWEIQQVVDADSKIV | M64;M7;M22;M54;M64;M7; |
| | MFLGTCRRCP | M6;M59 | QLSEISMDNS | M22;M54 |
| 1297 | EYFNSVCRLMKTIGPD | M17;M5; | SMQGAVDINKLCEEM | M60;M54;M45;M49;M60; |
| | MFLGTCRRC | M6;M59 | LDNRATLQAI | M54;M45;M49 |
| 1298 | PEYFNSVCRLMKTIGP | M17;M5; | LSMQGAVDINKLCEE | M60;M54;M45;M49;M60; |
| | DMFLGTCRR | M6;M59 | MLDNRATLQA | M54;M45;M49 |
| 1299 | FNSVCRLMKTIGPDMF | M17;M5; | DDTLRVEAFEYYHTT | M29;M6;M59;M41;M29;M6; |
| | LGTCRRCPA | M6;M59 | DPSFLGRYMS | M59;M41 |
| 1300 | PAMHAASGNLLLDKR | M57;M7; | LRVEAFEYYHTTDPSF | M29;M6;M59;M41;M29;M6; |
| | TTCFSVAALT | M9;M50 | LGRYMSALN | M59;M41 |
| 1301 | SVYAWNRKRISNCVA | M7;M67; | DTLRVEAFEYYHTTD | M29;M6;M59;M41;M29;M6; |
| | DYSVLYNSAS | M27;M74 | PSFLGRYMSA | M59;M41 |
| 1302 | VYAWNRKRISNCVAD | M7;M67; | NDDTLRVEAFEYYHT | M29;M6;M59;M41;M29;M6; |
| | YSVLYNSASF | M27;M74 | TDPSFLGRYM | M59;M41 |
| 1303 | GTTLPKGFYAEGSRGG | M5;M42; | TLRVEAFEYYHTTDP | M29;M6;M59;M41;M29;M6; |
| | SQASSRSSS | M75;M45 | SFLGRYMSAL | M59;M41 |
| 1304 | TTLPKGFYAEGSRGGS | M5;M42; | RVEAFEYYHTTDPSFL | M29;M6;M59;M41;M29;M6; |
| | QASSRSSSR | M75;M45 | GRYMSALNH | M59;M41 |
| 1305 | SQPFLMDLEGKQGNF | M6;M46; | ADVFHLYLQYIRKLH | M71;M32;M52;M16;M35; |
| | KNLREFVFKN | M54;M59 | DELTGHMLDM | M46;M9;M47 |
| 1306 | KYKYFSGAMDTTSYR | M1;M67; | YFSGAMDTTSYREAA | M78;M1;M79;M5;M78;M1; |
| | EAACCHLAKA | M1;M67 | CCHLAKALND | M79;M5 |
| 1307 | FEPSTQYEYGTEDDYQ | M1;M67; | FSGAMDTTSYREAAC | M78;M1;M79;M5;M78;M1; |
| | GKPLEFGAT | M1;M67 | CHLAKALNDF | M79;M5 |
| 1308 | LYNKYKYFSGAMDTT | M1;M67; | GAMDTTSYREAACC | M78;M1;M79;M5;M78;M1; |
| | SYREAACCHL | M1;M67 | HLAKALNDFSN | M79;M5 |
| 1309 | EEFEPSTQYEYGTEDD | M1;M67; | SGAMDTTSYREAACC | M78;M1;M79;M5;M78;M1; |
| | YQGKPLEFG | M1;M67 | HLAKALNDFS | M79;M5 |
| 1310 | YNKYKYFSGAMDTTS | M1;M67; | EKFKEGVEFLRDGWE | M57;M4;M27;M20;M57;M4; |
| | YREAACCHLA | M1;M67 | IVKFISTCAC | M27;M20 |
| 1311 | EEEFEPSTQYEYGTED | M1;M67; | LDGFMGRIRSVYPVA | M68;M29;M6;M58;M68; |
| | DYQGKPLEF | M1;M67 | SPNECNQMCL | M29;M6;M58 |
| 1312 | YKYFSGAMDTTSYRE | M1;M67; | GFMGRIRSVYPVASP | M68;M29;M6;M58;M68; |
| | AACCHLAKAL | M1;M67 | NECNQMCLST | M29;M6;M58 |
| 1313 | EEEEFEPSTQYEYGTE | M1;M67; | DGFMGRIRSVYPVAS | M68;M29;M6;M58;M68; |
| | DDYQGKPLE | M1;M67 | PNECNQMCLS | M29;M6;M58 |
| 1314 | ALYNKYKYFSGAMDT | M1;M67; | FMGRIRSVYPVASPNE | M68;M29;M6;M58;M68; |
| | TSYREAACCH | M1;M67 | CNQMCLSTL | M29;M6;M58 |
| 1315 | EFEPSTQYEYGTEDDY | M1;M67; | KLDGFMGRIRSVYPV | M68;M29;M6;M58;M68; |
| | QGKPLEFGA | M1;M67 | ASPNECNQMC | M29;M6;M58 |
| 1316 | NKYKYFSGAMDTTSY | M1;M67; | VEVQPQLEMELTPVV | M56;M29;M60;M6;M56; |
| | REAACCHLAK | M1;M67 | QTIEVNSFSG | M29;M60;M6 |
| 1317 | GNYNYLYRLFRKSNL | M1;M53; | EVQPQLEMELTPVVQ | M56;M29;M60;M6;M56; |
| | KPFERDISTE | M17;M37 | TIEVNSFSGY | M29;M60;M6 |
| 1318 | KKRWQLALSKGVHFV | M50;M49; | VQPQLEMELTPVVQT | M56;M29;M60;M6;M56; |
| | CNLLLLFVTV | M8;M23 | IEVNSFSGYL | M29;M60;M6 |
| 1319 | TRQVVNVVTTKIALK | M29;M12; | KTLVATAEAELAKNV | M38;M29;M73;M6;M38; |
| | GGKIVNNWLK | M29;M12 | SLDNVLSTFI | M29;M73;M6 |
| 1320 | LVDFQVTIAEILLIIMR | M5;M8; | TACTDDNALAYYNTT | M21;M22;M27;M6;M21; |
| | TFKVSIWN | M61;M37 | KGGRFVLALL | M22;M27;M6 |
| 1321 | AASGNLLLDKRTTCFS | M50;M7; | ACTDDNALAYYNTT | M21;M22;M27;M6;M21; |
| | VAALTNNVA | M29;M9 | KGGRFVLALLS | M22;M27;M6 |
| 1322 | ASGNLLLDKRTTCFSV | M50;M7; | TQTACTDDNALAYY | M21;M22;M27;M6;M21; |
| | AALTNNVAF | M29;M9 | NTTKGGRFVLA | M22;M27;M6 |
| 1323 | VYYHKNNKSWMESEF | M5;M41; | QTACTDDNALAYYN | M21;M22;M27;M6;M21; |
| | RVYSSANNCT | M48;M70 | TTKGGRFVLAL | M22;M27;M6 |
| 1324 | FLGVYYHKNNKSWM | M41;M22; | TTQTACTDDNALAYY | M21;M22;M27;M6;M21; |
| | ESEFRVYSSAN | M28;M70 | NTTKGGRFVL | M22;M27;M6 |
| 1325 | YKRDAPAHISTIGVCS | M59;M6; | CTDDNALAYYNTTK | M21;M22;M27;M6;M21; |
| | MTDIAKKPT | M66;M70 | GGRFVLALLSD | M22;M27;M6 |
| 1326 | DYKRDAPAHISTIGVC | M59;M6; | GTTQTACTDDNALAY | M21;M22;M27;M6;M21; |
| | SMTDIAKKP | M66;M70 | YNTTKGGRFV | M22;M27;M6 |
| 1327 | TQTTETAHSCNVNRFN | M33;M25; | IAWYTERSEKSYELQ | M25;M30;M39;M34;M25; |
| | VAITRAKVG | M6;M59 | TPFEIKLAKK | M30;M39;M34 |
| 1328 | TTETAHSCNVNRFNV | M33;M25; | EIAWYTERSEKSYEL | M25;M30;M39;M34;M25; |
| | AITRAKVGIL | M6;M59 | QTPFEIKLAK | M30;M39;M34 |
| 1329 | QTTETAHSCNVNRFN | M33;M25; | SNLGMPSYCTGYREG | M50;M18;M45;M49;M50; |
| | VAITRAKVGI | M6;M59 | YLNSTNVTIA | M18;M45;M49 |
| 1330 | KPNELSRVLGLKTLAT | M22;M6; | LMSNLGMPSYCTGYR | M50;M18;M45;M49;M50; |
| | HGLAAVNSV | M22;M6 | EGYLNSTNVT | M18;M45;M49 |
| 1331 | IKKPNELSRVLGLKTL | M22;M6; | NLGMPSYCTGYREGY | M50;M18;M45;M49;M50; |
| | ATHGLAAVN | M22;M6 | LNSTNVTIAT | M18;M45;M49 |
| 1332 | LTIKKPNELSRVLGLK | M22;M6; | VLMSNLGMPSYCTGY | M50;M18;M45;M49;M50; |
| | TLATHGLAA | M22;M6 | REGYLNSTNV | M18;M45;M49 |
| 1333 | TIKKPNELSRVLGLKT | M22;M6; | MSNLGMPSYCTGYRE | M50;M18;M45;M49;M50; |
| | LATHGLAAV | M22;M6 | GYLNSTNVTI | M18;M45;M49 |
| 1334 | NELSRVLGLKTLATHG | M22;M6; | LGMPSYCTGYREGYL | M50;M18;M45;M49;M50; |
| | LAAVNSVPW | M22;M6 | NSTNVTIATY | M18;M45;M49 |
| 1335 | PNELSRVLGLKTLATH | M22;M6; | ALVYFLQSINFVRIIM | M4;M75;M24;M10;M60; |
| | GLAAVNSVP | M22;M6 | RLWLCWKCR | M25;M9;M23 |
| 1336 | SLTIKKPNELSRVLGL | M22;M6; | DDNALAYYNTTKGG | M21;M22;M6;M20;M21; |
| | KTLATHGLA | M22;M6 | RFVLALLSDLQ | M22;M6;M20 |
| 1337 | KKPNELSRVLGLKTLA | M22;M6; | FDVLKSEDAQGMDN | M64;M22;M6;M37;M64; |
| | THGLAAVNS | M22;M6 | LACEDLKPVSE | M22;M6;M37 |
| 1338 | ELSRVLGLKTLATHGL | M22;M6; | SKPSVEQRKQDDKKI | M65;M67;M28;M74;M65; |
| | AAVNSVPWD | M22;M6 | KACVEEVTTT | M67;M28;M74 |
| 1339 | LAYILFTRFFYVLGLA | M5;M45; | KPSVEQRKQDDKKIK | M65;M67;M28;M74;M65; |
| | AIMQLFFSY | M5;M45 | ACVEEVTTTL | M67;M28;M74 |
| 1340 | ILFTRFFYVLGLAAIM | M5;M45; | QEYADVFHLYLQYIR | M71;M32;M16;M61;M35; |
| | QLFFSYFAV | M5;M45 | KLHDELTGHM | M52;M9;M47 |
| 1341 | AYILFTRFFYVLGLAAI | M5;M45; | EYADVFHLYLQYIRK | M71;M32;M16;M61;M35; |
| | MQLFFSYF | M5;M45 | LHDELTGHML | M52;M9;M47 |
| 1342 | YILFTRFFYVLGLAAI | M5;M45; | NQEYADVFHLYLQYI | M71;M32;M16;M61;M35; |
| | MQLFFSYFA | M5;M45 | RKLHDELTGH | M52;M9;M47 |
| 1343 | WFLAYILFTRFFYVLG | M5;M45; | TRCLNRVCTNYMPYF | M69;M29;M6;M20;M69; |
| | LAAIMQLFF | M5;M45 | FTLLLQLCTF | M29;M6;M20 |
| 1344 | FLAYILFTRFFYVLGLA | M5;M45; | MNVLTLVYKVYYGN | M1;M41;M24;M37;M1;M41; |
| | AIMQLFFS | M5;M45 | ALDQAISMWAL | M24;M37 |
| 1345 | EWFLAYILFTRFFYVL | M5;M45; | RVWTLMNVLTLVYK | M1;M41;M24;M37;M1;M41; |
| | GLAAIMQLF | M5;M45 | VYYGNALDQAI | M24;M37 |
| 1346 | AEWFLAYILFTRFFYV | M5;M45; | LMNVLTLVYKVYYG | M1;M41;M24;M37;M1;M41; |
| | LGLAAIMQL | M5;M45 | NALDQAISMWA | M24;M37 |
| 1347 | VAEWFLAYILFTRFFY | M5;M45; | RRVWTLMNVLTLVY | M1;M41;M24;M37;M1;M41; |
| | VLGLAAIMQ | M5;M45 | KVYYGNALDQA | M24;M37 |
| 1348 | DPFLGVYYHKNNKSW | M21;M22; | WTLMNVLTLVYKVY | M1;M41;M24;M37;M1;M41; |
| | MESEFRVYSS | M28;M70 | YGNALDQAISM | M24;M37 |
| 1349 | QLTGYKKPASRELKVT | M57;M63; | TLMNVLTLVYKVYY | M1;M41;M24;M37;M1;M41; |
| | FFPDLNGDV | M57;M63 | GNALDQAISMW | M24;M37 |
| 1350 | NQLTGYKKPASRELK | M57;M63; | NVLTLVYKVYYGNA | M1;M41;M24;M37;M1;M41; |
| | VTFFPDLNGD | M57;M63 | LDQAISMWALI | M24;M37 |
| 1351 | DGKMKDLSPRWYFYY | M52;M32; | VWTLMNVLTLVYKV | M1;M41;M24;M37;M1;M41; |
| | LGTGPEAGLP | M6;M31 | YYGNALDQAIS | M24;M37 |
| 1352 | YICGDSTECSNLLLQY | M13;M41; | TARTVYDDGARRVW | M5;M11;M2;M20;M5;M11; |
| | GSFCTQLNR | M46;M48 | TLMNVLTLVYK | M2;M20 |
| 1353 | LFNKVTLADAGFIKQY | M21;M41; | AANFCALILAYCNKT | M79;M41;M48;M49;M79; |
| | GDCLGDIAA | M48;M70 | VGELGDVRET | M41;M48;M49 |
| 1354 | LLFNKVTLADAGFIKQ | M21;M41; | EAANFCALILAYCNK | M79;M41;M48;M49;M79; |
| | YGDCLGDIA | M48;M70 | TVGELGDVRE | M41;M48;M49 |
| 1355 | ALVSDVGDSAEVAVK | M14;M37; | ANFCALILAYCNKTV | M79;M41;M48;M49;M79; |
| | MFDAYVNTFS | M14;M37 | GELGDVRETM | M41;M48;M49 |
| 1356 | LVSDVGDSAEVAVKM | M14;M37; | DEFTPFDVVRQCSGV | M4;M46;M60;M54;M4;M46; |
| | FDAYVNTFSS | M14;M37 | TFQSAVKRTI | M60;M54 |
| 1357 | VVFVLWAHGFELTSM | M46;M27; | TSEDMLNPNYEDLLIR | M29;M9;M28;M24;M29;M9; |
| | KYFVKIGPER | M28;M66 | KSNHNFLVQ | M28;M24 |
| 1358 | ILLIIMRTFKVSIWNLD | M1;M5; | SVRVVTTFDSEYCRH | M43;M22;M48;M20;M43; |
| | YIINLIIK | M37;M20 | GTCERSEAGV | M22;M48;M20 |
| 1359 | AEILLIIMRTFKVSIWN | M1;M5; | GLFCLLNRYFRLTLG | M18;M56;M14;M37;M18; |
| | LDYIINLI | M37;M20 | VYDYLVSTQE | M56;M14;M37 |
| 1360 | LLIIMRTFKVSIWNLD | M1;M5; | LIDSYFVVKRHTFSNY | M71;M58;M16;M18;M15; |
| | YIINLIIKN | M37;M20 | QHEETIYNL | M52;M47;M20 |
| 1361 | EILLIIMRTFKVSIWNL | M1;M5; | NLIDSYFVVKRHTFSN | M71;M58;M16;M18;M15; |
| | DYIINLII | M37;M20 | YQHEETIYN | M52;M47;M20 |
| 1362 | KCTLKSFTVEKGIYQT | M75;M25; | DNLIDSYFVVKRHTFS | M71;M58;M16;M18;M15; |
| | SNFRVQPTE | M8;M23 | NYQHEETIY | M52;M47;M20 |
| 1363 | DKSVYYTSNPTTFHLD | M50;M45; | EDDNLIDSYFVVKRH | M71;M58;M16;M18;M15; |
| | GEVITFDNL | M50;M45 | TFSNYQHEET | M52;M47;M20 |
| 1364 | IKGLNNLNRGMVLGS | M12;M17; | DDNLIDSYFVVKRHT | M71;M58;M16;M18;M15; |
| | LAATVRLQAG | M12;M17 | FSNYQHEETI | M52;M47;M20 |
| 1365 | STDVVYRAFDIYNDK | M78;M79; | QQVVDADSKIVQLSEI | M7;M60;M54;M49;M7;M60; |
| | VAGFAKFLKT | M7;M41 | SMDNSPNLA | M54;M49 |
| 1366 | SLENVAFNVVNKGHF | M15;M24; | VVDADSKIVQLSEISM | M7;M60;M54;M49;M7;M60; |
| | DGQQGEVPVS | M16;M71 | DNSPNLAWP | M54;M49 |
| 1367 | LENVAFNVVNKGHFD | M15;M24; | QVVDADSKIVQLSEIS | M7;M60;M54;M49;M7;M60; |
| | GQQGEVPVSI | M16;M71 | MDNSPNLAW | M54;M49 |
| 1368 | SKKPRQKRTATKAYN | M78;M5; | SDIDITFLKKDAPYIV | M57;M30;M11;M58;M57; |
| | VTQAFGRRGP | M2;M70 | GDVVQEGVL | M30;M11;M58 |
| 1369 | ASKKPRQKRTATKAY | M78;M5; | VHVMSKHTDFSSEIIG | M26;M53;M48;M41;M26; |
| | NVTQAFGRRG | M2;M70 | YKAIDGGVT | M53;M48;M41 |
| 1370 | LYFIKGLNNLNRGMV | M12;M24; | DSMSYEDQDALFAYT | M71;M24;M55;M10;M53; |
| | LGSLAATVRL | M12;M24 | KRNVIPTITQ | M35;M9;M23 |
| 1371 | YLYFIKGLNNLNRGM | M12;M24; | SYEDQDALFAYTKRN | M71;M24;M55;M10;M53; |
| | VLGSLAATVR | M12;M24 | VIPTITQMNL | M35;M9;M23 |
| 1372 | DMILSLLSKGRLIIREN | M21;M41; | MSYEDQDALFAYTKR | M71;M24;M55;M10;M53; |
| | NRVVISSD | M9;M48 | NVIPTITQMN | M35;M9;M23 |
| 1373 | PSGTWLTYTGAIKLDD | M10;M7; | SMSYEDQDALFAYTK | M71;M24;M55;M10;M53; |
| | KDPNFKDQV | M41;M35 | RNVIPTITQM | M35;M9;M23 |
| 1374 | SRVLGLKTLATHGLA | M54;M6; | SSEIIGYKAIDGGVTR | M43;M26;M41;M48;M43; |
| | AVNSVPWDTI | M54;M6 | DIASTDTCF | M26;M41;M48 |
| 1375 | RVLGLKTLATHGLAA | M54;M6; | ISNEKQEILGTVSWNL | M75;M24;M9;M8;M75;M24; |
| | VNSVPWDTIA | M54;M6 | REMLAHAEE | M9;M8 |
| 1376 | VLGLKTLATHGLAAV | M54;M6; | SIISNEKQEILGTVSW | M75;M24;M9;M8;M75;M24; |
| | NSVPWDTIAN | M54;M6 | NLREMLAHA | M9;M8 |
| 1377 | VICTSEDMLNPNYEDL | M29;M28; | IISNEKQEILGTVSWN | M75;M24;M9;M8;M75;M24; |
| | LIRKSNHNF | M29;M28 | LREMLAHAE | M9;M8 |
| 1378 | CTSEDMLNPNYEDLLI | M29;M28; | AEVAVKMFDAYVNT | M70;M41;M8;M37;M70; |
| | RKSNHNFLV | M29;M28 | FSSTFNVPMEK | M41;M8;M37 |
| 1379 | ICTSEDMLNPNYEDLLI | M29;M28; | LAYYNTTKGGRFVLA | M33;M70;M6;M20;M33; |
| | RKSNHNFL | M29;M28 | LLSDLQDLKW | M70;M6;M20 |
| 1380 | YDNKLKAHKDKSAQC | M13;M21; | ALAYYNTTKGGRFVL | M33;M70;M6;M20;M33; |
| | FKMFYKGVIT | M63;M52 | ALLSDLQDLK | M70;M6;M20 |
| 1381 | NLLLLFVTVYSHLLLV | M42;M6; | VYSVIYLYLTFYLTND | M50;M60;M54;M48;M50; |
| | AAGLEAPFL | M43;M37 | VSFLAHIQW | M60;M54;M48 |
| 1382 | VRNLQHRLYECLYRN | M52;M2; | ANNTKGSLPINVIVFD | M7;M56;M26;M52;M7;M56; |
| | RDVDTDFVNE | M47;M35 | GKSKCEESS | M26;M52 |
| 1383 | ECSNLLLQYGSFCTQL | M13;M41; | NNTKGSLPINVIVEDG | M7;M56;M26;M52;M7;M56; |
| | NRALTGIAV | M43;M48 | KSKCEESSA | M26;M52 |
| 1384 | VFKNIDGYFKIYSKHT | M15;M52; | VTCGTTTLNGLWLDD | M79;M7;M9;M24;M79;M7; |
| | PINLVRDLP | M16;M71 | VVYCPRHVIC | M9;M24 |
| 1385 | REFVFKNIDGYFKIYS | M15;M52; | EGNFYGPFVDRQTAQ | M15;M46;M2;M58;M15; |
| | KHTPINLVR | M16;M71 | AAGTDTTITV | M46;M2;M58 |
| 1386 | FVFKNIDGYFKIYSKH | M15;M52; | YGPFVDRQTAQAAGT | M15;M46;M2;M58;M15; |
| | TPINLVRDL | M16;M71 | DTTITVNVLA | M46;M2;M58 |
| 1387 | FKNIDGYFKIYSKHTPI | M15;M52; | NFYGPFVDRQTAQAA | M15;M46;M2;M58;M15; |
| | NLVRDLPQ | M16;M71 | GTDTTITVNV | M46;M2;M58 |
| 1388 | EFVFKNIDGYFKIYSK | M15;M52; | GNFYGPFVDRQTAQA | M15;M46;M2;M58;M15; |
| | HTPINLVRD | M16;M71 | AGTDTTITVN | M46;M2;M58 |
| 1389 | LREFVFKNIDGYFKIYS | M15;M52; | FYGPFVDRQTAQAAG | M15;M46;M2;M58;M15; |
| | KHTPINLV | M16;M71 | TDTTITVNVL | M46;M2;M58 |
| 1390 | TPSFKKGAKLLHKPIV | M1;M27; | LEGNFYGPFVDRQTA | M15;M46;M2;M58;M15; |
| | WHVNNATNK | M1;M27 | QAAGTDTTIT | M46;M2;M58 |
| 1391 | PSFKKGAKLLHKPIVW | M1;M27; | LNLEEAARYMRSLKV | M5;M72;M16;M35;M5;M72; |
| | HVNNATNKA | M1;M27 | PATVSVSSPD | M16;M35 |
| 1392 | PNMLRIMASLVLARK | M5;M69; | HGLNLEEAARYMRSL | M5;M72;M16;M35;M5;M72; |
| | HTTCCSLSHR | M2;M35 | KVPATVSVSS | M16;M35 |
| 1393 | RLRAKHYVYIGDPAQ | M57;M30; | NLEEAARYMRSLKVP | M5;M72;M16;M35;M5;M72; |
| | LPAPRTLLTK | M40;M31 | ATVSVSSPDA | M16;M35 |
| 1394 | ARLRAKHYVYIGDPA | M57;M30; | GLNLEEAARYMRSLK | M5;M72;M16;M35;M5;M72; |
| | QLPAPRTLLT | M40;M31 | VPATVSVSSP | M16;M35 |
| 1395 | LRAKHYVYIGDPAQLP | M57;M30; | LEEAARYMRSLKVPA | M5;M72;M16;M35;M5;M72; |
| | APRTLLTKG | M40;M31 | TVSVSSPDAV | M16;M35 |
| 1396 | APLIELCVDEAGSKSPI | M64;M7; | THGLNLEEAARYMRS | M5;M72;M16;M35;M5;M72; |
| | QYIDIGNY | M21;M23 | LKVPATVSVS | M16;M35 |
| 1397 | LNRYFRLTLGVYDYL | M6;M31; | KDNSYFTEQPIDLVPN | M64;M55;M39;M60;M64; |
| | VSTQEFRYMN | M6;M31 | QPYPNASFD | M55;M39;M60 |
| 1398 | NRYFRLTLGVYDYLV | M6;M31; | DNSYFTEQPIDLVPNQ | M64;M55;M39;M60;M64; |
| | STQEFRYMNS | M6;M31 | PYPNASFDN | M55;M39;M60 |
| 1399 | TSDLATNNLVVMAYIT | M50;M12; | SLPINVIVFDGKSKCE | M50;M7;M56;M52;M50;M7; |
| | GGVVQLTSQ | M50;M12 | ESSAKSASV | M56;M52 |
| 1400 | SNHNFLVQAGNVQLR | M12;M58; | LPINVIVFDGKSKCEE | M50;M7;M56;M52;M50;M7; |
| | VIGHSMQNCV | M12;M58 | SSAKSASVY | M56;M52 |
| 1401 | NHNFLVQAGNVQLRV | M12;M58; | LKGGKIVNNWLKQLI | M57;M55;M56;M11;M57; |
| | IGHSMQNCVL | M12;M58 | KVTLVFLFVA | M55;M56;M11 |
| 1402 | HNFLVQAGNVQLRVI | M12;M58; | TTIVNGVRRSFYVYA | M5;M39;M11;M41;M5;M39; |
| | GHSMQNCVLK | M12;M58 | NGGKGFCKLH | M11;M41 |
| 1403 | YRARAGEAANFCALIL | M41;M48; | GVRRSFYVYANGGK | M5;M39;M11;M41;M5;M39; |
| | AYCNKTVGE | M41;M48 | GFCKLHNWNCV | M11;M41 |
| 1404 | RARAGEAANFCALILA | M41;M48; | IVNGVRRSFYVYANG | M5;M39;M11;M41;M5;M39; |
| | YCNKTVGEL | M41;M48 | GKGFCKLHNW | M11;M41 |
| 1405 | ARAGEAANFCALILAY | M41;M48; | NGVRRSFYVYANGG | M5;M39;M11;M41;M5;M39; |
| | CNKTVGELG | M41;M48 | KGFCKLHNWNC | M11;M41 |
| 1406 | SCVLSGHNLAKHCLH | M1;M37; | VNGVRRSFYVYANG | M5;M39;M11;M41;M5;M39; |
| | VVGPNVNKGE | M1;M37 | GKGFCKLHNWN | M11;M41 |
| 1407 | CVLSGHNLAKHCLHV | M1;M37; | TIVNGVRRSFYVYAN | M5;M39;M11;M41;M5;M39; |
| | VGPNVNKGED | M1;M37 | GGKGFCKLHN | M11;M41 |
| 1408 | TQYEYGTEDDYQGKP | M1;M37; | ARAGKASCTLSEQLD | M64;M83;M22;M28;M64; |
| | LEFGATSAAL | M1;M37 | FIDTKRGVYC | M83;M22;M28 |
| 1409 | GSCVLSGHNLAKHCL | M1;M37; | RAGKASCTLSEQLDFI | M64;M83;M22;M28;M64; |
| | HVVGPNVNKG | M1;M37 | DTKRGVYCC | M83;M22;M28 |
| 1410 | FGPTYLDGADVTKIKP | M53;M12; | LARAGKASCTLSEQL | M64;M83;M22;M28;M64; |
| | HNSHEGKTF | M53;M12 | DFIDTKRGVY | M83;M22;M28 |
| 1411 | AHISTIGVCSMTDIAK | M63;M9; | TKKAGGTTEMLAKA | M78;M1;M18;M37;M78;M1; |
| | KPTETICAP | M59;M70 | LRKVPTDNYIT | M18;M37 |
| 1412 | PAHISTIGVCSMTDIAK | M63;M9; | KAGGTTEMLAKALR | M78;M1;M18;M37;M78;M1; |
| | KPTETICA | M59;M70 | KVPTDNYITTY | M18;M37 |
| 1413 | SQRGGSYTNDKACPLI | M32;M6; | VIPTKKAGGTTEMLA | M78;M1;M18;M37;M78;M1; |
| | AAVITREVG | M32;M6 | KALRKVPTDN | M18;M37 |
| 1414 | RDVLVRGFGDSVEEV | M63;M70; | IPTKKAGGTTEMLAK | M78;M1;M18;M37;M78;M1; |
| | LSEARQHLKD | M63;M70 | ALRKVPTDNY | M18;M37 |
| 1415 | QPELDSFKEELDKYFK | M75;M63; | KKAGGTTEMLAKAL | M78;M1;M18;M37;M78;M1; |
| | NHTSPDVDL | M59;M70 | RKVPTDNYITT | M18;M37 |
| 1416 | VRDVLVRGFGDSVEE | M63;M70; | AGGTTEMLAKALRK | M78;M1;M18;M37;M78;M1; |
| | VLSEARQHLK | M63;M70 | VPTDNYITTYP | M18;M37 |
| 1417 | LQPELDSFKEELDKYF | M75;M63; | PTKKAGGTTEMLAKA | M78;M1;M18;M37;M78;M1; |
| | KNHTSPDVD | M59;M70 | LRKVPTDNYI | M18;M37 |
| 1418 | RNARNGVLITEGSVKG | M13;M30; | LLTLQQIELKFNPPAL | M48;M73;M58;M74;M48; |
| | LQPSVGPKQ | M22;M46 | QDAYYRARA | M73;M58;M74 |
| 1419 | ARNGVLITEGSVKGLQ | M13;M30; | QKIAEIPKEEVKPFITE | M10;M26;M28;M24;M10; |
| | PSVGPKQAS | M22;M46 | SKPSVEQR | M26;M28;M24 |
| 1420 | NARNGVLITEGSVKGL | M13;M30; | YFYTSKTTVASLINTL | M57;M29;M72;M68;M57; |
| | QPSVGPKQA | M22;M46 | NDLNETLVT | M29;M72;M68 |
| 1421 | KLDGVVCTEIDPKLDN | M6;M59; | GDIILKPANNSLKITEE | M26;M34;M28;M58;M26; |
| | YYKKDNSYF | M6;M59 | VGHTDLMA | M34;M28;M58 |
| 1422 | PTGVHAGTDLEGNFY | M6;M37; | SGKPVPYCYDTNVLE | M46;M30;M29;M35;M46; |
| | GPFVDRQTAQ | M6;M37 | GSVAYESLRP | M30;M29;M35 |
| 1423 | TGVHAGTDLEGNFYG | M6;M37; | VNSFSGYLKLTDNVY | M47;M9;M8;M23;M47;M9; |
| | PFVDRQTAQA | M6;M37 | IKNADIVEEA | M8;M23 |
| 1424 | TIKPVTYKLDGVVCTE | M6;M59; | NSFSGYLKLTDNVYI | M47;M9;M8;M23;M47;M9; |
| | IDPKLDNYY | M6;M59 | KNADIVEEAK | M8;M23 |
| 1425 | YKLDGVVCTEIDPKLD | M6;M59; | SVVLLSVLQQLRVESS | M34;M30;M11;M54;M34; |
| | NYYKKDNSY | M6;M59 | SKLWAQCVQ | M30;M11;M54 |
| 1426 | VTYKLDGVVCTEIDPK | M6;M59; | PPQTSITSAVLQSGFR | M1;M38;M81;M62;M1;M38; |
| | LDNYYKKDN | M6;M59 | KMAFPSGKV | M81;M62 |
| 1427 | GVHAGTDLEGNFYGP | M6;M37; | GLVEVEKGVLPQLEQ | M13;M28;M66;M74;M13; |
| | FVDRQTAQAA | M6;M37 | PYVFIKRSDA | M28;M66;M74 |
| 1428 | IKPVTYKLDGVVCTEI | M6;M59; | CGLVEVEKGVLPQLE | M13;M28;M66;M74;M13; |
| | DPKLDNYYK | M6;M59 | QPYVFIKRSD | M28;M66;M74 |
| 1429 | PVTYKLDGVVCTEIDP | M6;M59; | LVEVEKGVLPQLEQP | M13;M28;M66;M74;M13; |
| | KLDNYYKKD | M6;M59 | YVFIKRSDAR | M28;M66;M74 |
| 1430 | KPVTYKLDGVVCTEID | M6;M59; | TCGLVEVEKGVLPQL | M13;M28;M66;M74;M13; |
| | PKLDNYYKK | M6;M59 | EQPYVFIKRS | M28;M66;M74 |
| 1431 | LDGVVCTEIDPKLDNY | M6;M59; | SLPVLQVRDVLVRGF | M43;M63;M68;M70;M43; |
| | YKKDNSYFT | M6;M59 | GDSVEEVLSE | M63;M68;M70 |
| 1432 | SDGTGTIYTELEPPCRF | M6;M59; | LPVLQVRDVLVRGFG | M43;M63;M68;M70;M43; |
| | VTDTPKGP | M6;M59 | DSVEEVLSEA | M63;M68;M70 |
| 1433 | TYKLDGVVCTEIDPKL | M6;M59; | RSFYVYANGGKGFCK | M42;M39;M11;M41;M42; |
| | DNYYKKDNS | M6;M59 | LHNWNCVNCD | M39;M11;M41 |
| 1434 | DNALAYYNTTKGGRF | M6;M20; | VTRCLNRVCTNYMPY | M64;M69;M6;M20;M64; |
| | VLALLSDLQD | M6;M20 | FFTLLLQLCT | M69;M6;M20 |
| 1435 | KSDGTGTIYTELEPPCR | M6;M59; | QQIELKFNPPALQDA | M75;M48;M73;M74;M75; |
| | FVTDTPKG | M6;M59 | YYRARAGEAA | M48;M73;M74 |
| 1436 | TVTLLPAADLDDFSKQ | M55;M53; | LQQIELKFNPPALQDA | M75;M48;M73;M74;M75; |
| | LQQSMSSAD | M58;M71 | YYRARAGEA | M48;M73;M74 |
| 1437 | HDFFKFRIDGDMVPHI | M55;M26; | LSVLQQLRVESSSKL | M11;M34;M30;M82;M11; |
| | SRQRLTKYT | M10;M71 | WAQCVQLHND | M34;M30;M82 |
| 1438 | IDGDMVPHISRQRLTK | M55;M26; | LLSVLQQLRVESSSKL | M11;M34;M30;M82;M11; |
| | YTMADLVYA | M10;M71 | WAQCVQLHN | M34;M30;M82 |
| 1439 | FAFACPDGVKHVYQL | M46;M39; | KKSLNVAKSEFDRDA | M50;M7;M32;M68;M50;M7; |
| | RARSVSPKLF | M60;M54 | AMQRKLEKMA | M32;M68 |
| 1440 | FACPDGVKHVYQLRA | M46;M39; | KSLNVAKSEFDRDAA | M50;M7;M32;M68;M50;M7; |
| | RSVSPKLFIR | M60;M54 | MQRKLEKMAD | M32;M68 |
| 1441 | ACPDGVKHVYQLRAR | M46;M39; | KHSSGVTRELMRELN | M60;M30;M22;M54;M60; |
| | SVSPKLFIRQ | M60;M54 | GGAYTRYVDN | M30;M22;M54 |
| 1442 | QFAFACPDGVKHVYQ | M46;M39; | NTKHSSGVTRELMRE | M60;M30;M22;M54;M60; |
| | LRARSVSPKL | M60;M54 | LNGGAYTRYV | M30;M22;M54 |
| 1443 | AFACPDGVKHVYQLR | M46;M39; | TKHSSGVTRELMREL | M60;M30;M22;M54;M60; |
| | ARSVSPKLFI | M60;M54 | NGGAYTRYVD | M30;M22;M54 |
| 1444 | CPDGVKHVYQLRARS | M46;M39; | TERSEKSYELQTPFEI | M25;M34;M39;M24;M25; |
| | VSPKLFIRQE | M60;M54 | KLAKKFDTF | M34;M39;M24 |
| 1445 | LDNLRANNTKGSLPIN | M7;M53; | EAARVVRSIFSRTLET | M42;M34;M2;M59;M42; |
| | VIVFDGKSK | M7;M53 | AQNSVRVLQ | M34;M2;M59 |
| 1446 | FISPYNSQNAVASKILG | M21;M39; | EILGTVSWNLREMLA | M75;M9;M28;M24;M75;M9; |
| | LPTQTVDS | M26;M54 | HAEETRKLMP | M28;M24 |
| 1447 | RKAVFISPYNSQNAVA | M21;M39; | KQEILGTVSWNLREM | M75;M9;M28;M24;M75;M9; |
| | SKILGLPTQ | M26;M49 | LAHAEETRKL | M28;M24 |
| 1448 | LTRNPAWRKAVFISPY | M21;M39; | QEILGTVSWNLREML | M75;M9;M28;M24;M75;M9; |
| | NSQNAVASK | M26;M49 | AHAEETRKLM | M28;M24 |
| 1449 | AEIPKEEVKPFITESKP | M26;M28; | ILGTVSWNLREMLAH | M75;M9;M28;M24;M75;M9; |
| | SVEQRKQD | M26;M28 | AEETRKLMPV | M28;M24 |
| 1450 | PKEEVKPFITESKPSVE | M26;M28; | EKQEILGTVSWNLRE | M75;M9;M28;M24;M75;M9; |
| | QRKQDDKK | M26;M28 | MLAHAEETRK | M28;M24 |
| 1451 | EIPKEEVKPFITESKPS | M26;M28; | GARFYFYTSKTTVAS | M15;M29;M72;M41;M15; |
| | VEQRKQDD | M26;M28 | LINTLNDLNE | M29;M72;M41 |
| 1452 | KIAEIPKEEVKPFITESK | M26;M28; | KKLKKSLNVAKSEFD | M33;M68;M32;M28;M33; |
| | PSVEQRK | M26;M28 | RDAAMQRKLE | M68;M32;M28 |
| 1453 | KEEVKPFITESKPSVEQ | M26;M28; | LKKLKKSLNVAKSEF | M33;M68;M32;M28;M33; |
| | RKQDDKKI | M26;M28 | DRDAAMQRKL | M68;M32;M28 |
| 1454 | IPKEEVKPFITESKPSV | M26;M28; | DVFHLYLQYIRKLHD | M71;M32;M52;M38;M35; |
| | EQRKQDDK | M26;M28 | ELTGHMLDMY | M46;M9;M47 |
| 1455 | IAEIPKEEVKPFITESKP | M26;M28; | VTFFPDLNGDVVAID | M7;M69;M60;M37;M7;M69; |
| | SVEQRKQ | M26;M28 | YKHYTPSFKK | M60;M37 |
| 1456 | EEVKPFITESKPSVEQR | M26;M28; | HEIAWYTERSEKSYE | M25;M34;M30;M49;M25; |
| | KQDDKKIK | M26;M28 | LQTPFEIKLA | M34;M30;M49 |
| 1457 | LRANNTKGSLPINVIVF | M7;M56; | TEVNEFACVVADAVI | M78;M25;M34;M39;M78; |
| | DGKSKCEE | M7;M56 | KTLQPVSELL | M25;M34;M39 |
| 1458 | RANNTKGSLPINVIVF | M7;M56; | LLNKEMYLKLRSDVL | M42;M39;M43;M61;M42; |
| | DGKSKCEES | M7;M56 | LPLTQYNRYL | M39;M43;M61 |
| 1459 | VVLSFELLHAPATVCG | M46;M34; | TFLLNKEMYLKLRSD | M42;M39;M43;M61;M42; |
| | PKKSTNLVK | M39;M27 | VLLPLTQYNR | M39;M43;M61 |
| 1460 | QNAVVKIYCPACHNSE | M35;M28; | FLLNKEMYLKLRSDV | M42;M39;M43;M61;M42; |
| | VGPEHSLAE | M35;M28 | LLPLTQYNRY | M39;M43;M61 |
| 1461 | YLPQNAVVKIYCPACH | M35;M28; | PNPKGFCDLKGKYVQ | M42;M39;M43;M31;M42; |
| | NSEVGPEHS | M35;M28 | IPTTCANDPV | M39;M43;M31 |
| 1462 | PQNAVVKIYCPACHNS | M35;M28; | TYYLFDESGEFKLAS | M15;M52;M16;M23;M15; |
| | EVGPEHSLA | M35;M28 | HMYCSFYPPD | M52;M16;M23 |
| 1463 | LPQNAVVKIYCPACHN | M35;M28; | GVDAVNLLTNMFTPL | M15;M52;M16;M62;M15; |
| | SEVGPEHSL | M35;M28 | IQPIGALDIS | M52;M16;M62 |
| 1464 | ESKPSVEQRKQDDKKI | M28;M74; | CGVDAVNLLTNMFTP | M15;M52;M16;M62;M15; |
| | KACVEEVTT | M28;M74 | LIQPIGALDI | M52;M16;M62 |
| 1465 | TESKPSVEQRKQDDK | M28;M74; | FCGVDAVNLLTNMFT | M15;M52;M16;M62;M15; |
| | KIKACVEEVT | M28;M74 | PLIQPIGALD | M52;M16;M62 |
| 1466 | RRSFYVYANGGKGFC | M39;M11; | DAVNLLTNMFTPLIQP | M15;M52;M16;M62;M15; |
| | KLHNWNCVNC | M39;M11 | IGALDISAS | M52;M16;M62 |
| 1467 | ITGRLQSLQTYVTQQLI | M46;M29; | VDAVNLLTNMFTPLI | M15;M52;M16;M62;M15; |
| | RAAEIRAS | M22;M49 | QPIGALDISA | M52;M16;M62 |
| 1468 | GDEVRQIAPGQTGKIA | M80;M67; | GAKLKALNLGETFVT | M55;M18;M27;M24;M55; |
| | DYNYKLPDD | M38;M62 | HSKGLYRKCV | M18;M27;M24 |
| 1469 | DEVRQIAPGQTGKIAD | M80;M67; | GGAKLKALNLGETFV | M55;M18;M27;M24;M55; |
| | YNYKLPDDF | M38;M62 | THSKGLYRKC | M18;M27;M24 |
| 1470 | CVSFCYMHHMELPTG | M43;M28; | IELKFNPPALQDAYYR | M75;M39;M73;M74;M75; |
| | VHAGTDLEGN | M43;M28 | ARAGEAANF | M39;M73;M74 |
| 1471 | VGLMWLSYFIASFRLF | M15;M35; | QIELKFNPPALQDAY | M75;M39;M73;M74;M75; |
| | ARTRSMWSF | M26;M16 | YRARAGEAAN | M39;M73;M74 |
| 1472 | LVGLMWLSYFIASFRL | M15;M35; | SCNNYMLTYNKVEN | M15;M52;M11;M74;M15; |
| | FARTRSMWS | M26;M16 | MTPRDLGACID | M52;M11;M74 |
| 1473 | LVIGAVILRGHLRIAG | M55;M53; | AFEKMVSLLSVLLSM | M69;M67;M22;M61;M69; |
| | HHLGRCDIK | M58;M17 | QGAVDINKLC | M67;M22;M61 |
| 1474 | ELVIGAVILRGHLRIAG | M55;M53; | EAFEKMVSLLSVLLS | M69;M67;M22;M61;M69; |
| | HHLGRCDI | M58;M17 | MQGAVDINKL | M67;M22;M61 |
| 1475 | VIGAVILRGHLRIAGH | M55;M53; | FEKMVSLLSVLLSMQ | M69;M67;M22;M61;M69; |
| | HLGRCDIKD | M58;M17 | GAVDINKLCE | M67;M22;M61 |
| 1476 | IGAVILRGHLRIAGHH | M55;M53; | QEGVVDYGARFYFYT | M15;M29;M40;M41;M15; |
| | LGRCDIKDL | M58;M17 | SKTTVASLIN | M29;M40;M41 |
| 1477 | CPACHNSEVGPEHSLA | M68;M28; | DTIANYAKPFLNKVV | M78;M55;M15;M23;M78; |
| | EYHNESGLK | M68;M28 | STTTNIVTRC | M55;M15;M23 |
| 1478 | YCPACHNSEVGPEHSL | M68;M28; | WDTIANYAKPFLNKV | M78;M55;M15;M23;M78; |
| | AEYHNESGL | M68;M28 | VSTTTNIVTR | M55;M15;M23 |
| 1479 | AVVKIYCPACHNSEVG | M68;M28; | GTGQAITVTPEANMD | M78;M34;M29;M24;M78; |
| | PEHSLAEYH | M68;M28 | QESFGGASCC | M34;M29;M24 |
| 1480 | IYCPACHNSEVGPEHS | M68;M28; | AQEAYEQAVANGDS | M33;M13;M59;M55;M33; |
| | LAEYHNESG | M68;M28 | EVVLKKLKKSL | M13;M59;M55 |
| 1481 | VVKIYCPACHNSEVGP | M68;M28; | RYMSALNHTKKWKY | M5;M24;M54;M74;M5;M24; |
| | EHSLAEYHN | M68;M28 | PQVNGLTSIKW | M54;M74 |
| 1482 | VKIYCPACHNSEVGPE | M68;M28; | VLECNVKTTEVVGDII | M26;M29;M58;M53;M26; |
| | HSLAEYHNE | M68;M28 | LKPANNSLK | M29;M58;M53 |
| 1483 | KIYCPACHNSEVGPEH | M68;M28; | LECNVKTTEVVGDIIL | M26;M29;M58;M53;M26; |
| | SLAEYHNES | M68;M28 | KPANNSLKI | M29;M58;M53 |
| 1484 | EAEVQIDRLITGRLQSL | M46;M29; | GRYMSALNHTKKWK | M5;M24;M25;M74;M5;M24; |
| | QTYVTQQL | M22;M38 | YPQVNGLTSIK | M25;M74 |
| 1485 | AEVQIDRLITGRLQSL | M46;M29; | SEDMLNPNYEDLLIR | M21;M29;M9;M24;M21; |
| | QTYVTQQLI | M22;M38 | KSNHNFLVQA | M29;M9;M24 |
| 1486 | VEAEVQIDRLITGRLQ | M46;M29; | GDVVAIDYKHYTPSF | M69;M82;M16;M37;M69; |
| | SLQTYVTQQ | M22;M38 | KKGAKLLHKP | M82;M16;M37 |
| 1487 | KDLLARAGKASCTLSE | M83;M28; | CVNCDTFCAGSTFISD | M57;M35;M70;M20;M57; |
| | QLDFIDTKR | M83;M28 | EVARDLSLQ | M35;M70;M20 |
| 1488 | TTTIQTIVEVQPQLEME | M28;M59; | YGARFYFYTSKTTVA | M15;M29;M47;M41;M15; |
| | LTPVVQTI | M28;M59 | SLINTLNDLN | M29;M47;M41 |
| 1489 | EDNQTTTIQTIVEVQP | M28;M59; | SSFLEMKSEKQVEQKI | M25;M34;M24;M74;M25; |
| | QLEMELTPV | M28;M59 | AEIPKEEVK | M34;M24;M74 |
| 1490 | DNQTTTIQTIVEVQPQ | M28;M59; | VTCAKEIKESVQTFFK | M43;M53;M24;M17;M43; |
| | LEMELTPVV | M28;M59 | LVNKFLALC | M53;M24;M17 |
| 1491 | DLLARAGKASCTLSEQ | M83;M28; | TCAKEIKESVQTFFKL | M43;M53;M24;M17;M43; |
| | LDFIDTKRG | M83;M28 | VNKFLALCA | M53;M24;M17 |
| 1492 | SEARQHLKDGTCGLV | M13;M28; | EAYEQAVANGDSEV | M55;M13;M53;M59;M55; |
| | EVEKGVLPQL | M13;M28 | VLKKLKKSLNV | M13;M53;M59 |
| 1493 | QTTTIQTIVEVQPQLE | M28;M59; | YEQAVANGDSEVVL | M55;M13;M53;M59;M55; |
| | MELTPVVQT | M28;M59 | KKLKKSLNVAK | M13;M53;M59 |
| 1494 | LLARAGKASCTLSEQL | M83;M28; | AYEQAVANGDSEVV | M55;M13;M53;M59;M55; |
| | DFIDTKRGV | M83;M28 | LKKLKKSLNVA | M13;M53;M59 |
| 1495 | NQTTTIQTIVEVQPQLE | M28;M59; | QYELKHGTFTCASEY | M33;M65;M38;M66;M33; |
| | MELTPVVQ | M28;M59 | TGNYQCGHYK | M65;M38;M66 |
| 1496 | TSDYYQLYSTQLSTDT | M42;M41; | ELKHGTFTCASEYTG | M33;M65;M38;M66;M33; |
| | GVEHVTFFI | M39;M20 | NYQCGHYKHI | M65;M38;M66 |
| 1497 | KKVDGVVQQLPETYF | M33;M71; | YELKHGTFTCASEYT | M33;M65;M38;M66;M33; |
| | TQSRNLQEFK | M54;M31 | GNYQCGHYKH | M65;M38;M66 |
| 1498 | KVDGVVQQLPETYFT | M33;M71; | LKHGTFTCASEYTGN | M33;M65;M38;M66;M33; |
| | QSRNLQEFKP | M54;M31 | YQCGHYKHIT | M65;M38;M66 |
| 1499 | ADIVVFDEISMATNYD | M50;M18; | INAQVAKSHNIALIW | M32;M72;M68;M37;M32; |
| | LSVVNARLR | M33;M55 | NVKDFMSLSE | M72;M68;M37 |
| 1500 | DSLSSTASALGKLQDV | M79;M29; | AQNSVRVLQKAAITIL | M5;M9;M54;M24;M5;M9; |
| | VNQNAQALN | M22;M47 | DGISQYSLR | M54;M24 |
| 1501 | QDSLSSTASALGKLQD | M79;M29; | QMAPISAMVRMYIFF | M23;M55;M9;M20;M23; |
| | VVNQNAQAL | M22;M47 | ASFYYVWKSY | M55;M9;M20 |
| 1502 | TGVLTESNKKFLPFQQ | M78;M41; | MAPISAMVRMYIFFA | M23;M55;M9;M20;M23; |
| | FGRDIADTT | M61;M71 | SFYYVWKSYV | M55;M9;M20 |
| 1503 | LTESNKKFLPFQQFGR | M78;M41; | LIINLVQMAPISAMVR | M23;M55;M9;M20;M23; |
| | DIADTTDAV | M61;M71 | MYIFFASFY | M55;M9;M20 |
| 1504 | TESNKKFLPFQQFGRD | M78;M41; | VQMAPISAMVRMYIF | M23;M55;M9;M20;M23; |
| | LADTTDAVR | M61;M71 | FASFYYVWKS | M55;M9;M20 |
| 1505 | VLTESNKKFLPFQQFG | M78;M41; | NLVQMAPISAMVRM | M23;M55;M9;M20;M23; |
| | RDIADTTDA | M61;M71 | YIFFASFYYVW | M55;M9;M20 |
| 1506 | ESNKKFLPFQQFGRDI | M78;M41; | INLVQMAPISAMVRM | M23;M55;M9;M20;M23; |
| | ADTTDAVRD | M61;M71 | YIFFASFYYV | M55;M9;M20 |
| 1507 | GVLTESNKKFLPFQQF | M78;M41; | IINLVQMAPISAMVR | M23;M55;M9;M20;M23; |
| | GRDIADTTD | M61;M71 | MYIFFASFYY | M55;M9;M20 |
| 1508 | QITISSFKWDLTAFGLV | M30;M82; | WLIINLVQMAPISAM | M23;M55;M9;M20;M23; |
| | AEWFLAYI | M30;M82 | VRMYIFFASF | M55;M9;M20 |
| 1509 | ETIQITISSFKWDLTAF | M30;M82; | LVQMAPISAMVRMYI | M23;M55;M9;M20;M23; |
| | GLVAEWFL | M30;M82 | FFASFYYVWK | M55;M9;M20 |
| 1510 | LETIQITISSFKWDLTA | M30;M82; | TGDLQPLEQPTSEAVE | M60;M54;M58;M17;M60; |
| | FGLVAEWF | M30;M82 | APLVGTPVC | M54;M58;M17 |
| 1511 | IQITISSFKWDLTAFGL | M30;M82; | LLEDEFTPFDVVRQCS | M46;M60;M54;M41;M46; |
| | VAEWFLAY | M30;M82 | GVTFQSAVK | M60;M54;M41 |
| 1512 | SLETIQITISSFKWDLT | M30;M82; | ALLEDEFTPFDVVRQ | M46;M60;M54;M41;M46; |
| | AFGLVAEW | M30;M82 | CSGVTFQSAV | M60;M54;M41 |
| 1513 | TIQITISSFKWDLTAFG | M30;M82; | LEDEFTPFDVVRQCS | M46;M60;M54;M41;M46; |
| | LVAEWFLA | M30;M82 | GVTFQSAVKR | M60;M54;M41 |
| 1514 | ALLLLDRLNQLESKMS | M15;M53; | SALLEDEFTPFDVVRQ | M46;M60;M54;M41;M46; |
| | GKGQQQQGQ | M11;M58 | CSGVTFQSA | M60;M54;M41 |
| 1515 | LALLLLDRLNQLESKM | M15;M53; | SCKRVLNVVCKTCGQ | M15;M5;M52;M58;M15;M5; |
| | SGKGQQQQG | M11;M58 | QQTTLKGVEA | M52;M58 |
| 1516 | LGKLQDVVNQNAQAL | M79;M53; | LNHTKKWKYPQVNG | M5;M60;M54;M74;M5;M60; |
| | NTLVKQLSSN | M22;M15 | LTSIKWADNNC | M54;M74 |
| 1517 | PPALQDAYYRARAGE | M75;M39; | NHTKKWKYPQVNGL | M5;M60;M54;M74;M5;M60; |
| | AANFCALILA | M75;M39 | TSIKWADNNCY | M54;M74 |
| 1518 | LKFNPPALQDAYYRA | M75;M39; | ALNHTKKWKYPQVN | M5;M60;M54;M74;M5;M60; |
| | RAGEAANFCA | M75;M39 | GLTSIKWADNN | M54;M74 |
| 1519 | NPPALQDAYYRARAG | M75;M39; | ASWVMRIMTWLDMV | M64;M83;M22;M70;M64; |
| | EAANFCALIL | M75;M39 | DTSLSGFKLKD | M83;M22;M70 |
| 1520 | FNPPALQDAYYRARA | M75;M39; | PASWVMRIMTWLDM | M64;M83;M22;M70;M64; |
| | GEAANFCALI | M75;M39 | VDTSLSGFKLK | M83;M22;M70 |
| 1521 | KFNPPALQDAYYRAR | M75;M39; | QVEQKIAEIPKEEVKP | M10;M26;M24;M74;M10; |
| | AGEAANFCAL | M75;M39 | FITESKPSV | M26;M24;M74 |
| 1522 | PALQDAYYRARAGEA | M75;M39; | VEQKIAEIPKEEVKPFI | M10;M26;M24;M74;M10; |
| | ANFCALILAY | M75;M39 | TESKPSVE | M26;M24;M74 |
| 1523 | DKNTQEVFAQVKQIY | M55;M18; | KQVEQKIAEIPKEEVK | M10;M26;M24;M74;M10; |
| | KTPPIKDFGG | M32;M56 | PFITESKPS | M26;M24;M74 |
| 1524 | VGMQKYSTLQGPPGT | M42;M34; | DWLEEKFKEGVEFLR | M57;M9;M27;M20;M57;M9; |
| | GKSHFAIGLA | M29;M41 | DGWEIVKFIS | M27;M20 |
| 1525 | GMQKYSTLQGPPGTG | M42;M34; | PVLDWLEEKFKEGVE | M57;M9;M27;M20;M57;M9; |
| | KSHFAIGLAL | M29;M41 | FLRDGWEIVK | M27;M20 |
| 1526 | AYYVGYLQPRTFLLK | M15;M11; | KPVLDWLEEKFKEGV | M57;M9;M27;M20;M57;M9; |
| | YNENGTITDA | M58;M47 | EFLRDGWEIV | M27;M20 |
| 1527 | DGQVDLFRNARNGVL | M33;M42; | LDWLEEKFKEGVEFL | M57;M9;M27;M20;M57;M9; |
| | ITEGSVKGLQ | M22;M43 | RDGWEIVKFI | M27;M20 |
| 1528 | LMYKGLPWNVVRIKI | M57;M46; | VLDWLEEKFKEGVEF | M57;M9;M27;M20;M57;M9; |
| | VQMLSDTLKN | M9;M20 | LRDGWEIVKF | M27;M20 |
| 1529 | MYKGLPWNVVRIKIV | M57;M46; | EHFIETISLAGSYKDW | M64;M32;M29;M68;M64; |
| | QMLSDTLKNL | M9;M20 | SYSGQSTQL | M32;M29;M68 |
| 1530 | HYTPSFKKGAKLLHKP | M27;M16; | HFIETISLAGSYKDWS | M64;M32;M29;M68;M64; |
| | IVWHVNNAT | M27;M16 | YSGQSTQLG | M32;M29;M68 |
| 1531 | YKHYTPSFKKGAKLL | M27;M16; | EEHFIETISLAGSYKD | M64;M32;M29;M68;M64; |
| | HKPIVWHVNN | M27;M16 | WSYSGQSTQ | M32;M29;M68 |
| 1532 | KHYTPSFKKGAKLLH | M27;M16; | ADIVEEAKKVKPTVV | M18;M11;M73;M23;M18; |
| | KPIVWHVNNA | M27;M16 | VNAANVYLKH | M11;M73;M23 |
| 1533 | KVGGSCVLSGHNLAK | M80;M37; | ECNVKTTEVVGDIILK | M26;M34;M58;M53;M26; |
| | HCLHVVGPNV | M80;M37 | PANNSLKIT | M34;M58;M53 |
| 1534 | PLKVGGSCVLSGHNL | M80;M37; | NVKTTEVVGDIILKPA | M26;M34;M58;M53;M26; |
| | AKHCLHVVGP | M80;M37 | NNSLKITEE | M34;M58;M53 |
| 1535 | LKVGGSCVLSGHNLA | M80;M37; | CNVKTTEVVGDIILKP | M26;M34;M58;M53;M26; |
| | KHCLHVVGPN | M80;M37 | ANNSLKITE | M34;M58;M53 |
| 1536 | HPNQEYADVFHLYLQ | M71;M32; | IDTKRGVYCCREHEH | M75;M27;M66;M74;M75; |
| | YIRKLHDELT | M9;M61 | EIAWYTERSE | M27;M66;M74 |
| 1537 | KHPNQEYADVFHLYL | M71;M32; | FIDTKRGVYCCREHE | M75;M27;M66;M74;M75; |
| | QYIRKLHDEL | M9;M61 | HEIAWYTERS | M27;M66;M74 |
| 1538 | HDELTGHMLDMYSV | M34;M22; | LDFIDTKRGVYCCRE | M75;M27;M66;M74;M75; |
| | MLTNDNTSRYW | M38;M31 | HEHEIAWYTE | M27;M66;M74 |
| 1539 | KYTMADLVYALRHFD | M10;M52; | DFIDTKRGVYCCREH | M75;M27;M66;M74;M75; |
| | EGNCDTLKEI | M17;M49 | EHEIAWYTER | M27;M66;M74 |
| 1540 | TKYTMADLVYALRHF | M10;M52; | QLDFIDTKRGVYCCR | M75;M27;M66;M74;M75; |
| | DEGNCDTLKE | M17;M49 | EHEHEIAWYT | M27;M66;M74 |
| 1541 | LTKYTMADLVYALRH | M10;M52; | RIMTWLDMVDTSLSG | M64;M75;M83;M22;M64; |
| | FDEGNCDTLK | M17;M49 | FKLKDCVMYA | M75;M83;M22 |
| 1542 | YTMADLVYALRHFDE | M10;M52; | IMTWLDMVDTSLSGF | M64;M75;M83;M22;M64; |
| | GNCDTLKEIL | M17;M49 | KLKDCVMYAS | M75;M83;M22 |
| 1543 | QRLTKYTMADLVYAL | M10;M52; | MRIMTWLDMVDTSL | M64;M75;M83;M22;M64; |
| | RHFDEGNCDT | M17;M49 | SGFKLKDCVMY | M75;M83;M22 |
| 1544 | MADLVYALRHFDEGN | M10;M52; | YRKCVKSREETGLLM | M60;M54;M38;M31;M60; |
| | CDTLKEILVT | M17;M49 | PLKAPKEIIF | M54;M38;M31 |
| 1545 | TMADLVYALRHFDEG | M10;M52; | KCVKSREETGLLMPL | M60;M54;M38;M31;M60; |
| | NCDTLKEILV | M17;M49 | KAPKEIIFLE | M54;M38;M31 |
| 1546 | RLTKYTMADLVYALR | M10;M52; | GLYRKCVKSREETGL | M60;M54;M38;M31;M60; |
| | HFDEGNCDTL | M17;M49 | LMPLKAPKEI | M54;M38;M31 |
| 1547 | YHYQECVRGTTVLLK | M57;M27; | CVKSREETGLLMPLK | M60;M54;M38;M31;M60; |
| | EPCSSGTYEG | M58;M20 | APKEIIFLEG | M54;M38;M31 |
| 1548 | YQECVRGTTVLLKEPC | M57;M27; | RKCVKSREETGLLMP | M60;M54;M38;M31;M60; |
| | SSGTYEGNS | M58;M20 | LKAPKEIIFL | M54;M38;M31 |
| 1549 | QECVRGTTVLLKEPCS | M57;M27; | LYRKCVKSREETGLL | M60;M54;M38;M31;M60; |
| | SGTYEGNSP | M58;M20 | MPLKAPKEII | M54;M38;M31 |
| 1550 | HYQECVRGTTVLLKEP | M57;M27; | DYKAFKQIVESCGNF | M42;M41;M25;M23;M42; |
| | CSSGTYEGN | M58;M20 | KVTKGKAKKG | M41;M25;M23 |
| 1551 | LYHYQECVRGTTVLL | M57;M27; | YKAFKQIVESCGNFK | M42;M41;M25;M23;M42; |
| | KEPCSSGTYE | M58;M20 | VTKGKAKKGA | M41;M25;M23 |
| 1552 | VSGTNGTKRFDNPVLP | M78;M65; | KAFKQIVESCGNFKV | M42;M41;M25;M23;M42; |
| | FNDGVYFAS | M82;M66 | TKGKAKKGAW | M41;M25;M23 |
| 1553 | IHVSGTNGTKRFDNPV | M78;M65; | FCAGSTFISDEVARDL | M35;M31;M70;M20;M35; |
| | LPFNDGVYF | M82;M66 | SLQFKRPIN | M31;M70;M20 |
| 1554 | AIHVSGTNGTKRFDNP | M78;M65; | DTFCAGSTFISDEVAR | M35;M31;M70;M20;M35; |
| | VLPFNDGVY | M82;M66 | DLSLQFKRP | M31;M70;M20 |
| 1555 | GTNGTKRFDNPVLPFN | M78;M65; | TFCAGSTFISDEVARD | M35;M31;M70;M20;M35; |
| | DGVYFASTE | M82;M66 | LSLQFKRPI | M31;M70;M20 |
| 1556 | TNGTKRFDNPVLPFND | M78;M65; | INGLMLLEIKDTEKYC | M78;M9;M82;M61;M78;M9; |
| | GVYFASTEK | M82;M66 | ALAPNMMVT | M82;M61 |
| 1557 | HVSGTNGTKRFDNPV | M78;M65; | NGLMLLEIKDTEKYC | M78;M34;M9;M82;M78; |
| | LPFNDGVYFA | M82;M66 | ALAPNMMVTN | M34;M9;M82 |
| 1558 | SGTNGTKRFDNPVLPF | M78;M65; | LNDNLLEILQKEKVNI | M10;M9;M27;M24;M10;M9; |
| | NDGVYFAST | M82;M66 | NIVGDFKLN | M27;M24 |
| 1559 | HNQDVNLHSSRLSFKE | M57;M53; | NLLEILQKEKVNINIV | M10;M9;M27;M24;M10;M9; |
| | LLVYAADPA | M38;M20 | GDFKLNEEI | M27;M24 |
| 1560 | GTGVLTESNKKFLPFQ | M78;M24; | NDNLLEILQKEKVNIN | M10;M9;M27;M24;M10;M9; |
| | QFGRDIADT | M61;M41 | IVGDFKLNE | M27;M24 |
| 1561 | YSLLLCRMNSRNYIAQ | M10;M34; | GLNDNLLEILQKEKV | M10;M9;M27;M24;M10;M9; |
| | VDVVNFNLT | M54;M24 | NINIVGDFKL | M27;M24 |
| 1562 | HNWNCVNCDTFCAGS | M57;M70; | DNLLEILQKEKVNINI | M10;M9;M27;M24;M10;M9; |
| | TFISDEVARD | M57;M70 | VGDFKLNEE | M27;M24 |
| 1563 | NCVNCDTFCAGSTFIS | M57;M70; | GPLSAQTGIAVLDMC | M10;M32;M24;M59;M10; |
| | DEVARDLSL | M57;M70 | ASLKELLQNG | M32;M24;M59 |
| 1564 | WNCVNCDTFCAGSTFI | M57;M70; | PLSAQTGIAVLDMCA | M10;M32;M24;M59;M10; |
| | SDEVARDLS | M57;M70 | SLKELLQNGM | M32;M24;M59 |
| 1565 | NWNCVNCDTFCAGST | M57;M70; | GADVTKIKPHNSHEG | M42;M21;M53;M37;M42; |
| | FISDEVARDL | M57;M70 | KTFYVLPNDD | M21;M53;M37 |
| 1566 | GYLTSSSKTPEEHFIETI | M40;M29; | TIANYAKPFLNKVVS | M78;M9;M15;M23;M78;M9; |
| | SLAGSYK | M40;M29 | TTTNIVTRCL | M15;M23 |
| 1567 | QTLLALHRSYLTPGDS | M23;M5; | DMCASLKELLQNGM | M50;M18;M56;M60;M50; |
| | SSGWTAGAA | M24;M70 | NGRTILGSALL | M18;M56;M60 |
| 1568 | VLQQLRVESSSKLWA | M11;M82; | ATAQEAYEQAVANG | M33;M13;M47;M59;M33; |
| | QCVQLHNDIL | M11;M82 | DSEVVLKKLKK | M13;M47;M59 |
| 1569 | LQQLRVESSSKLWAQ | M11;M82; | FATAQEAYEQAVAN | M33;M13;M47;M59;M33; |
| | CVQLHNDILL | M11;M82 | GDSEVVLKKLK | M13;M47;M59 |
| 1570 | QQLRVESSSKLWAQC | M11;M82; | YFCTCYFGLFCLLNR | M60;M56;M18;M37;M60; |
| | VQLHNDILLA | M11;M82 | YFRLTLGVYD | M56;M18;M37 |
| 1571 | WLIVGVALLAVFQSAS | M60;M26; | GYFCTCYFGLFCLLN | M60;M56;M18;M37;M60; |
| | KIITLKKRW | M61;M35 | RYFRLTLGVY | M56;M18;M37 |
| 1572 | LIVGVALLAVFQSASK | M60;M26; | TLLTKGTLEPEYFNSV | M4;M13;M12;M16;M19;M1; |
| | IITLKKRWQ | M61;M35 | CRLMKTIGP | M3;M6;M15 |
| 1573 | IVGVALLAVFQSASKII | M60;M26; | EPLVDLPIGINITRFQT | M4;M21;M5;M1;M3;M2; |
| | TLKKRWQL | M61;M35 | LLALHRSY | M26;M22;M20 |
| 1574 | GDCEEEEFEPSTQYEY | M33;M67; | PLVDLPIGINITRFQTL | M4;M21;M5;M1;M3;M2; |
| | GTEDDYQGK | M33;M67 | LALHRSYL | M26;M22;M20 |
| 1575 | NGLTGTGVLTESNKKF | M78;M24; | NLWNTFTRLQSLENV | M4;M54;M14;M1;M34;M25; |
| | LPFQQFGRD | M38;M61 | AFNVVNKGHF | M60;M3;M39 |
| 1576 | FNGLTGTGVLTESNKK | M78;M24; | LAKRFKESPFELEDFIP | M4;M19;M1;M25;M3;M61; |
| | FLPFQQFGR | M38;M61 | MDSTVKNY | M18;M43;M20 |
| 1577 | DIVVFDEISMATNYDL | M50;M18; | KRFKESPFELEDFIPM | M4;M19;M1;M25;M3;M61; |
| | SVVNARLRA | M25;M33 | DSTVKNYFI | M18;M43;M20 |
| 1578 | HLYLQYIRKLHDELTG | M46;M32; | AKRFKESPFELEDFIP | M4;M19;M1;M25;M3;M61; |
| | HMLDMYSVM | M52;M38 | MDSTVKNYF | M18;M43;M20 |
| 1579 | IQEGVVDYGARFYFYT | M29;M41; | CFVDDIVKTDGTLMIE | M9;M10;M1;M5;M7;M3;M6; |
| | SKTTVASLI | M29;M41 | RFVSLAIDA | M8;M11 |
| 1580 | NRATRVECTTIVNGVR | M29;M41; | LWNTFTRLQSLENVA | M4;M54;M24;M1;M34;M25; |
| | RSFYVYANG | M29;M41 | FNVVNKGHFD | M60;M3;M39 |
| 1581 | IKQYGDCLGDIAARDL | M78;M79; | TFTRLQSLENVAFNV | M4;M54;M16;M24;M1;M60; |
| | ICAQKFNGL | M50;M65 | VNKGHFDGQQ | M3;M27;M15 |
| 1582 | FIKQYGDCLGDIAARD | M78;M65; | FKELLVYAADPAMH | M64;M12;M30;M48;M33; |
| | LICAQKFNG | M79;M50 | AASGNLLLDKR | M36;M8;M39;M44 |
| 1583 | FPREGVFVSNGTHWF | M50;M18; | LYALVYFLQSINFVRII | M4;M54;M24;M19;M60; |
| | VTQRNFYEPQ | M24;M31 | MRLWLCWK | M25;M3;M9;M23 |
| 1584 | REGVFVSNGTHWFVT | M50;M18; | VLPFNDGVYFASTEK | M64;M12;M28;M14;M10; |
| | QRNFYEPQII | M24;M31 | SNIIRGWIFG | M24;M6;M45;M22 |
| 1585 | PREGVFVSNGTHWFV | M50;M18; | PVLPFNDGVYFASTE | M64;M12;M28;M14;M10; |
| | TQRNFYEPQI | M24;M31 | KSNIIRGWIF | M24;M6;M45;M22 |
| 1586 | YKGIKIQEGVVDYGAR | M41;M67; | LPFNDGVYFASTEKS | M64;M12;M28;M14;M10; |
| | FYFYTSKTT | M41;M67 | NIIRGWIFGT | M24;M6;M45;M22 |
| 1587 | KGIKIQEGVVDYGARF | M41;M67; | NPVLPFNDGVYFAST | M64;M12;M28;M14;M10; |
| | YFYTSKTTV | M41;M67 | EKSNIIRGWI | M24;M6;M45;M22 |
| 1588 | AGFIKQYGDCLGDIAA | M50;M65; | DNPVLPFNDGVYFAS | M64;M12;M28;M14;M10; |
| | RDLICAQKF | M27;M70 | TEKSNIIRGW | M24;M6;M45;M22 |
| 1589 | GFIKQYGDCLGDIAAR | M78;M65; | RDVDTDFVNEFYAYL | M4;M71;M16;M3;M18;M35; |
| | DLICAQKFN | M50;M70 | RKHFSMMILS | M15;M52;M26 |
| 1590 | IEFLKRGDKSVYYTSN | M50;M22; | SFPQSAPHGVVFLHV | M64;M21;M28;M66;M70; |
| | PTTFHLDGE | M50;M22 | TYVPAQEKNF | M53;M3;M26;M9 |
| 1591 | PKYKFVRIQPGQTFSV | M29;M27; | LCEKALKYLPIDKCSR | M71;M13;M28;M14;M29; |
| | LACYNGSPS | M29;M27 | IIPARARVE | M51;M33;M18;M22 |
| 1592 | VKMLCTHTGTGQAIT | M78;M29; | CEKALKYLPIDKCSRII | M71;M13;M28;M14;M29; |
| | VTPEANMDQE | M78;M29 | PARARVEC | M51;M33;M18;M22 |
| 1593 | CVKMLCTHTGTGQAI | M78;M29; | AGDYILANTCTERLK | M56;M73;M19;M31;M6; |
| | TVTPEANMDQ | M78;M29 | LFAAETLKAT | M44;M18;M63;M47 |
| 1594 | NVANYQKVGMQKYS | M42;M21; | ELLVYAADPAMHAA | M12;M24;M57;M50;M33; |
| | TLQGPPGTGKS | M34;M41 | SGNLLLDKRTT | M36;M8;M46;M39 |
| 1595 | GTAVMSLKEGQINDMI | M60;M54; | KLQFTSLEIPRRNVAT | M28;M54;M29;M2;M6;M45; |
| | LSLLSKGRL | M59;M49 | LQAENVTGL | M27;M59;M20 |
| 1596 | DKSAFVNLKQLPFFYY | M60;M32; | TLEPEYFNSVCRLMK | M13;M12;M16;M14;M5;M6; |
| | SDSPCESHG | M41;M54 | TIGPDMFLGT | M18;M17;M15 |
| 1597 | NKKDWYDFVENPDIL | M43;M21; | KEIDRLNEVAKNLNE | M11;M70;M78;M65;M2; |
| | RVYANLGERV | M32;M49 | SLIDLQELGK | M79;M51;M67;M82 |
| 1598 | TLVATAEAELAKNVSL | M29;M73; | AIATCDWTNAGDYIL | M56;M73;M19;M31;M6; |
| | DNVLSTFIS | M29;M73 | ANTCTERLKL | M44;M18;M52;M47 |
| 1599 | GGDGKMKDLSPRWYF | M52;M32; | IATCDWTNAGDYILA | M56;M73;M19;M31;M6; |
| | YYLGTGPEAG | M60;M31 | NTCTERLKLF | M44;M18;M52;M47 |
| 1600 | MVSLLSVLLSMQGAV | M69;M67; | ATCDWTNAGDYILAN | M56;M73;M19;M31;M6; |
| | DINKLCEEML | M69;M67 | TCTERLKLFA | M44;M18;M52;M47 |
| 1601 | EKMVSLLSVLLSMQG | M69;M67; | NAIATCDWTNAGDYI | M56;M73;M19;M31;M6; |
| | AVDINKLCEE | M69;M67 | LANTCTERLK | M44;M18;M52;M47 |
| 1602 | KMVSLLSVLLSMQGA | M69;M67; | YNLWNTFTRLQSLEN | M4;M54;M14;M1;M34;M25; |
| | VDINKLCEEM | M69;M67 | VAFNVVNKGH | M60;M42;M39 |
| 1603 | IIGGAKLKALNLGETF | M27;M24; | SLWVYKQFDTYNLW | M71;M58;M16;M14;M19; |
| | VTHSKGLYR | M27;M24 | NTFTRLQSLEN | M25;M41;M17;M15 |
| 1604 | IIIGGAKLKALNLGETF | M27;M24; | GLPYGANKDGIIWVA | M64;M40;M14;M34;M30; |
| | VTHSKGLY | M27;M24 | TEGALNTPKD | M6;M57;M49;M22 |
| 1605 | SYLFQHANLDSCKRVL | M5;M27; | IDRLNEVAKNLNESLI | M11;M70;M78;M65;M61; |
| | NVVCKTCGQ | M5;M27 | DLQELGKYE | M79;M51;M67;M82 |
| 1606 | MSYLFQHANLDSCKR | M5;M27; | VDTVSALVYDNKLK | M21;M28;M16;M7;M38; |
| | VLNVVCKTCG | M5;M27 | AHKDKSAQCFK | M50;M63;M17;M35 |
| 1607 | KSPNFSKLINIIIWFLLL | M74;M31; | PAEIVDTVSALVYDN | M12;M28;M16;M7;M38; |
| | SVCLGSL | M74;M31 | KLKAHKDKSA | M50;M17;M35;M59 |
| 1608 | LKSPNFSKLINIIIWFLL | M74;M31; | VYAADPAMHAASGN | M12;M24;M57;M50;M33; |
| | LSVCLGS | M74;M31 | LLLDKRTTCFS | M8;M46;M39;M9 |
| 1609 | PVCVETKAIVSTIQRK | M5;M60; | YAADPAMHAASGNL | M12;M24;M57;M50;M33; |
| | YKGIKIQEG | M5;M60 | LLDKRTTCFSV | M8;M46;M39;M9 |
| 1610 | VCVETKAIVSTIQRKY | M5;M60; | LHSSRLSFKELLVYAA | M64;M48;M57;M36;M41; |
| | KGIKIQEGV | M5;M60 | DPAMHAASG | M49;M26;M22;M20 |
| 1611 | CVETKAIVSTIQRKYK | M5;M60; | HSSRLSFKELLVYAA | M64;M48;M57;M36;M41; |
| | GIKIQEGVV | M5;M60 | DPAMHAASGN | M49;M26;M22;M20 |
| 1612 | MPVCVETKAIVSTIQR | M5;M60; | EVFNATRFASVYAWN | M19;M16;M55;M25;M53; |
| | KYKGIKIQE | M5;M60 | RKRISNCVAD | M2;M74;M27;M20 |
| 1613 | KEMYLKLRSDVLLPLT | M53;M61; | FNATRFASVYAWNR | M19;M16;M55;M25;M53; |
| | QYNRYLALY | M53;M61 | KRISNCVADYS | M2;M74;M27;M20 |
| 1614 | DKAGQKTYERHSLSH | M31;M70; | VFNATRFASVYAWN | M19;M16;M55;M25;M53; |
| | FVNLDNLRAN | M31;M70 | RKRISNCVADY | M2;M74;M27;M20 |
| 1615 | DTTSYREAACCHLAK | M78;M79; | NATRFASVYAWNRK | M19;M16;M55;M25;M53; |
| | ALNDFSNSGS | M78;M79 | RISNCVADYSV | M2;M74;M27;M20 |
| 1616 | SYREAACCHLAKALN | M78;M79; | LNGNWYDFGDFIQTT | M4;M40;M9;M58;M16;M34; |
| | DFSNSGSDVL | M78;M79 | PGSGVPVVDS | M8;M15;M11 |
| 1617 | TTSYREAACCHLAKA | M78;M79; | DLNGNWYDFGDFIQT | M4;M40;M9;M58;M16;M34; |
| | LNDFSNSGSD | M78;M79 | TPGSGVPVVD | M8;M15;M11 |
| 1618 | YREAACCHLAKALND | M78;M79; | NGNWYDFGDFIQTTP | M4;M40;M9;M58;M16;M34; |
| | FSNSGSDVLY | M78;M79 | GSGVPVVDSY | M8;M15;M11 |
| 1619 | AGALNKATNNAMQV | M78;M79; | TCCSLSHRFYRLANE | M28;M5;M30;M2;M79;M42; |
| | ESDDYIATNGP | M78;M79 | CAQVLSEMVM | M43;M46;M39 |
| 1620 | REAACCHLAKALNDF | M78;M79; | CSLSHRFYRLANECA | M28;M5;M30;M2;M79;M42; |
| | SNSGSDVLYQ | M78;M79 | QVLSEMVMCG | M43;M46;M39 |
| 1621 | AACCHLAKALNDFSN | M78;M79; | CCSLSHRFYRLANEC | M28;M5;M30;M2;M79;M42; |
| | SGSDVLYQPP | M78;M79 | AQVLSEMVMC | M43;M46;M39 |
| 1622 | TSYREAACCHLAKAL | M78;M79; | PPPGDQFKHLIPLMYK | M56;M19;M12;M25;M60; |
| | NDFSNSGSDV | M78;M79 | GLPWNVVRI | M18;M15;M39;M47 |
| 1623 | EAACCHLAKALNDFS | M78;M79; | DNQDLNGNWYDFGD | M4;M40;M9;M58;M16;M70; |
| | NSGSDVLYQP | M78;M79 | FIQTTPGSGVP | M34;M8;M11 |
| 1624 | SNLLLQYGSFCTQLNR | M13;M41; | SNFRVQPTESIVRFPNI | M21;M28;M31;M34;M30; |
| | ALTGIAVEQ | M43;M20 | TNLCPFGE | M48;M41;M26;M22 |
| 1625 | NLLLQYGSFCTQLNRA | M13;M41; | HTTCCSLSHRFYRLA | M28;M5;M60;M30;M2;M79; |
| | LTGIAVEQD | M43;M20 | NECAQVLSEM | M43;M42;M39 |
| 1626 | DVLVRGFGDSVEEVLS | M10;M70; | TTCCSLSHRFYRLANE | M28;M5;M60;M30;M2;M79; |
| | EARQHLKDG | M10;M70 | CAQVLSEMV | M43;M42;M39 |
| 1627 | VLVRGFGDSVEEVLSE | M10;M70; | LPYGANKDGIIWVAT | M64;M40;M34;M30;M6; |
| | ARQHLKDGT | M10;M70 | EGALNTPKDH | M57;M72;M49;M22 |
| 1628 | WVPRASANIGCNHTG | M9;M38; | PYGANKDGIIWVATE | M64;M40;M34;M30;M6; |
| | VVGEGSEGLN | M9;M38 | GALNTPKDHI | M57;M72;M49;M22 |
| 1629 | YWVPRASANIGCNHT | M9;M38; | YGANKDGIIWVATEG | M64;M40;M34;M30;M6; |
| | GVVGEGSEGL | M9;M38 | ALNTPKDHIG | M57;M72;M49;M22 |
| 1630 | AYWVPRASANIGCNH | M9;M38; | GANKDGIIWVATEGA | M64;M40;M34;M30;M6; |
| | TGVVGEGSEG | M9;M38 | LNTPKDHIGT | M57;M72;M49;M22 |
| 1631 | VPRASANIGCNHTGVV | M9;M38; | RGTTTYKLNVGDYFV | M16;M34;M18;M41;M43; |
| | GEGSEGLND | M9;M38 | LTSHTVMPLS | M15;M42;M39;M44 |
| 1632 | ETLPTEVLTEEVVLKT | M38;M62; | GTTTYKLNVGDYFVL | M16;M34;M18;M41;M43; |
| | GDLQPLEQP | M38;M62 | TSHTVMPLSA | M15;M42;M39;M44 |
| 1633 | ARSEDKRAKVTSAMQ | M38;M62; | LVTLAILTALRLCAYC | M54;M16;M60;M25;M63; |
| | TMLFTMLRKL | M38;M62 | CNIVNVSLV | M41;M17;M46;M39 |
| 1634 | KQARSEDKRAKVTSA | M38;M62; | VTLAILTALRLCAYCC | M54;M16;M60;M25;M63; |
| | MQTMLFTMLR | M38;M62 | NIVNVSLVK | M41;M17;M46;M39 |
| 1635 | GETLPTEVLTEEVVLK | M38;M62; | LLQYGSFCTQLNRAL | M13;M28;M14;M70;M41; |
| | TGDLQPLEQ | M38;M62 | TGIAVEQDKN | M67;M27;M43;M20 |
| 1636 | NSGSDVLYQPPQTSITS | M38;M62; | LQYGSFCTQLNRALT | M13;M28;M14;M70;M41; |
| | AVLQSGFR | M38;M62 | GIAVEQDKNT | M67;M27;M43;M20 |
| 1637 | QARSEDKRAKVTSAM | M38;M62; | DYPKCDRAMPNMLRI | M21;M12;M30;M69;M41; |
| | QTMLFTMLRK | M38;M62 | MASLVLARKH | M46;M39;M22;M23 |
| 1638 | RSEDKRAKVTSAMQT | M38;M62; | ILANTCTERLKLFAAE | M64;M40;M21;M48;M63; |
| | MLFTMLRKLD | M38;M62 | TLKATEETF | M49;M26;M22;M23 |
| 1639 | TLPTEVLTEEVVLKTG | M38;M62; | SGINASVVNIQKEIDR | M28;M14;M70;M78;M61; |
| | DLQPLEQPT | M38;M62 | LNEVAKNLN | M79;M35;M82;M47 |
| 1640 | NKCAYWVPRASANIG | M75;M38; | FKVSIWNLDYIINLIIK | M56;M73;M58;M16;M50; |
| | CNHTGVVGEG | M75;M38 | NLSKSLTE | M18;M17;M11;M44 |
| 1641 | HNKCAYWVPRASANI | M75;M38; | VDGVVQQLPETYFTQ | M71;M32;M54;M16;M31; |
| | GCNHTGVVGE | M75;M38 | SRNLQEFKPR | M45;M33;M35;M47 |
| 1642 | KHCLHVVGPNVNKGE | M60;M77; | FLHVTYVPAQEKNFT | M28;M66;M78;M65;M2; |
| | DIQLLKSAYE | M60;M77 | TAPAICHDGK | M79;M33;M26;M82 |
| 1643 | GEFKLASHMYCSFYPP | M23;M20; | DSNGTITVEELKKLLE | M4;M58;M16;M55;M53; |
| | DEDEEEGDC | M23;M20 | QWNLVIGFL | M59;M17;M52;M47 |
| 1644 | SGEFKLASHMYCSFYP | M23;M20; | ATRFASVYAWNRKRI | M16;M55;M25;M53;M2; |
| | PDEDEEEGD | M23;M20 | SNCVADYSVL | M74;M67;M27;M20 |
| 1645 | SGHNLAKHCLHVVGP | M77;M37; | KLDDKDPNFKDQVIL | M56;M73;M53;M44;M17; |
| | NVNKGEDIQL | M77;M37 | LNKHIDAYKT | M35;M52;M11;M47 |
| 1646 | HNLAKHCLHVVGPNV | M77;M37; | DDKDPNFKDQVILLN | M56;M73;M53;M44;M17; |
| | NKGEDIQLLK | M77;M37 | KHIDAYKTFP | M35;M52;M11;M47 |
| 1647 | GHNLAKHCLHVVGPN | M77;M37; | LDDKDPNFKDQVILL | M56;M73;M53;M44;M17; |
| | VNKGEDIQLL | M77;M37 | NKHIDAYKTF | M35;M52;M11;M47 |
| 1648 | IVKTDGTLMIERFVSL | M4;M1;M5; | DKDPNFKDQVILLNK | M56;M73;M53;M44;M17; |
| | AIDAYPLTK | M2;M3 | HIDAYKTFPP | M35;M52;M11;M47 |
| 1649 | VKTDGTLMIERFVSLA | M4;M1;M5; | IKLDDKDPNFKDQVIL | M56;M73;M53;M44;M17; |
| | IDAYPLTKH | M2;M3 | LNKHIDAYK | M35;M52;M11;M47 |
| 1650 | DIVKTDGTLMIERFVS | M4;M1;M5; | VVQQLPETYFTQSRN | M71;M75;M32;M16;M31; |
| | LAIDAYPLT | M2;M3 | LQEFKPRSQM | M45;M33;M35;M47 |
| 1651 | NSPFHPLADNKFALTC | M4;M14; | GVVQQLPETYFTQSR | M71;M75;M32;M16;M31; |
| | FSTQFAFAC | M1;M3;M22 | NLQEFKPRSQ | M45;M33;M35;M47 |
| 1652 | YSSANNCTFEYVSQPF | M4;M1;M3; | QQLPETYFTQSRNLQ | M71;M75;M32;M16;M31; |
| | LMDLEGKQG | M57;M20 | EFKPRSQMEI | M45;M33;M35;M47 |
| 1653 | VYSSANNCTFEYVSQP | M4;M1;M3; | VQQLPETYFTQSRNL | M71;M75;M32;M16;M31; |
| | FLMDLEGKQ | M57;M20 | QEFKPRSQME | M45;M33;M35;M47 |
| 1654 | HSIGFDYVYNPFMIDV | M4;M1;M3; | QLPETYFTQSRNLQEF | M71;M75;M32;M16;M31; |
| | QQWGFTGNL | M57;M20 | KPRSQMEID | M45;M33;M35;M47 |
| 1655 | SGDATTAYANSVFNIC | M4;M1;M29; | LPETYFTQSRNLQEFK | M71;M75;M32;M16;M31; |
| | QAVTANVNA | M3;M39 | PRSQMEIDF | M45;M33;M35;M47 |
| 1656 | SSGDATTAYANSVFNI | M4;M1;M29; | SNVTWFHAIHVSGTN | M64;M40;M29;M45;M49; |
| | CQAVTANVN | M3;M39 | GTKRFDNPVL | M15;M26;M22;M23 |
| 1657 | TVDSSQGSEYDYVIFT | M4;M3;M51; | FSNVTWFHAIHVSGT | M64;M40;M29;M45;M49; |
| | QTTETAHSC | M22;M23 | NGTKRFDNPV | M15;M26;M22;M23 |
| 1658 | PFHPLADNKFALTCFS | M4;M21;M14; | PFFSNVTWFHAIHVSG | M64;M40;M29;M45;M49; |
| | TQFAFACPD | M3;M22 | TNGTKRFDN | M15;M26;M22;M23 |
| 1659 | HPLADNKFALTCFSTQ | M4;M21;M3; | FFSNVTWFHAIHVSG | M64;M40;M29;M45;M49; |
| | FAFACPDGV | M6;M22 | TNGTKRFDNP | M15;M26;M22;M23 |
| 1660 | LYRNRDVDTDFVNEF | M4;M6;M3; | INASVVNIQKEIDRLN | M28;M70;M78;M65;M2; |
| | YAYLRKHFSM | M26;M59 | EVAKNLNES | M61;M79;M82;M47 |
| 1661 | CLYRNRDVDTDFVNE | M4;M6;M3; | GINASVVNIQKEIDRL | M28;M70;M78;M65;M2; |
| | FYAYLRKHFS | M26;M59 | NEVAKNLNE | M61;M79;M82;M47 |
| 1662 | GWHNMLKTVYSDVE | M6;M45;M3; | IHADQLTPTWRVYST | M75;M58;M24;M16;M5;M6; |
| | NPHLMGWDYPK | M43;M59 | GSNVFQTRAG | M41;M17;M82 |
| 1663 | HNMLKTVYSDVENPH | M6;M45;M3; | FVENPDILRVYANLG | M64;M11;M21;M32;M48; |
| | LMGWDYPKCD | M43;M59 | ERVRQALLKT | M41;M49;M46;M22 |
| 1664 | WHNMLKTVYSDVENP | M6;M45;M3; | DFVENPDILRVYANL | M64;M11;M21;M32;M48; |
| | HLMGWDYPKC | M43;M59 | GERVRQALLK | M41;M49;M46;M22 |
| 1665 | ADNKFALTCFSTQFAF | M21;M3;M6; | YDFVENPDILRVYAN | M64;M11;M21;M32;M48; |
| | ACPDGVKHV | M33;M22 | LGERVRQALL | M41;M49;M46;M22 |
| 1666 | LADNKFALTCFSTQFA | M21;M3;M6; | QAWQPGVAMPNLYK | M12;M24;M55;M10;M53; |
| | FACPDGVKH | M33;M22 | MQRMLLEKCDL | M17;M52;M9;M23 |
| 1667 | DNKFALTCFSTQFAFA | M21;M3;M6; | SQAWQPGVAMPNLY | M12;M24;M55;M10;M53; |
| | CPDGVKHVY | M33;M22 | KMQRMLLEKCD | M17;M52;M9;M23 |
| 1668 | PNCVNCLDDRCILHCA | M64;M24; | LHVTYVPAQEKNFTT | M66;M78;M65;M29;M2; |
| | NFNVLFSTV | M3;M48;M23 | APAICHDGKA | M79;M33;M26;M82 |
| 1669 | IAARDLICAQKFNGLT | M58;M78; | TFSNYQHEETIYNLLK | M40;M34;M30;M41;M42; |
| | VLPPLLTDE | M3;M79;M39 | DCPAVAKHD | M27;M43;M39;M11 |
| 1670 | DCVVLHSYFTSDYYQ | M3;M41;M43; | FSNYQHEETIYNLLKD | M40;M34;M30;M41;M42; |
| | LYSTQLSTDT | M42;M39 | CPAVAKHDF | M27;M43;M39;M11 |
| 1671 | KDCVVLHSYFTSDYY | M3;M41;M43; | VAMPNLYKMQRMLL | M12;M55;M53;M17;M35; |
| | QLYSTQLSTD | M42;M39 | EKCDLQNYGDS | M52;M9;M47;M23 |
| 1672 | AVDALCEKALKYLPID | M71;M1;M51; | GVAMPNLYKMQRML | M12;M55;M53;M17;M35; |
| | KCSRIIPAR | M18;M22 | LEKCDLQNYGD | M52;M9;M47;M23 |
| 1673 | PPTSFGPLVRKIFVDGV | M71;M7;M1; | TTTYKLNVGDYFVLT | M16;M25;M34;M18;M41; |
| | PFVVSTGY | M36;M11 | SHTVMPLSAP | M43;M15;M42;M39 |
| 1674 | ACSHAAVDALCEKAL | M1;M60;M51; | YADVFHLYLQYIRKL | M71;M32;M52;M16;M61; |
| | KYLPIDKCSR | M49;M22 | HDELTGHMLD | M35;M46;M9;M47 |
| 1675 | HAAVDALCEKALKYL | M1;M60;M51; | DEDDNLIDSYFVVKR | M71;M58;M16;M54;M18; |
| | PIDKCSRIIP | M49;M22 | HTFSNYQHEE | M15;M52;M47;M20 |
| 1676 | SHAAVDALCEKALKY | M1;M60;M51; | VGVALLAVFQSASKII | M58;M24;M55;M60;M61; |
| | LPIDKCSRII | M49;M22 | TLKKRWQLA | M17;M35;M26;M9 |
| 1677 | CSHAAVDALCEKALK | M1;M60;M51; | GVALLAVFQSASKIIT | M58;M24;M55;M60;M61; |
| | YLPIDKCSRI | M49;M22 | LKKRWQLAL | M17;M35;M26;M9 |
| 1678 | AAVDALCEKALKYLPI | M1;M60;M51; | YEDQDALFAYTKRNV | M71;M24;M55;M10;M53; |
| | DKCSRIIPA | M49;M22 | IPTITQMNLK | M35;M9;M59;M23 |
| 1679 | YTACSHAAVDALCEK | M1;M60;M51; | VDDIVKTDGTLMIERF | M4;M9;M10;M1;M5;M2; |
| | ALKYLPIDKC | M67;M49 | VSLAIDAYP | M3;M6;M8;M11 |
| 1680 | VYTACSHAAVDALCE | M1;M60;M51; | LVDLPIGINITRFQTLL | M4;M21;M24;M5;M1;M3; |
| | KALKYLPIDK | M67;M49 | ALHRSYLT | M2;M26;M22;M20 |
| 1681 | QGSEYDYVIFTQTTET | M64;M6;M51; | KQIYKTPPIKDFGGFN | M4;M32;M28;M14;M31; |
| | AHSCNVNRF | M22;M23 | FSQILPDPS | M1;M3;M33;M27;M35 |
| 1682 | GSEYDYVIFTQTTETA | M64;M6;M51; | CPFGEVFNATRFASV | M4;M19;M58;M55;M25; |
| | HSCNVNRFN | M22;M59 | YAWNRKRISN | M53;M3;M2;M17;M20 |
| 1683 | SQGSEYDYVIFTQTTE | M4;M64;M51; | WNTFTRLQSLENVAF | M4;M54;M24;M1;M34;M25; |
| | TAHSCNVNR | M22;M23 | NVVNKGHFDG | M60;M3;M27;M39 |
| 1684 | KSNLKPFERDISTEIYQ | M53;M36; | NTFTRLQSLENVAFN | M4;M54;M24;M1;M34;M25; |
| | AGSTPCNG | M17;M46;M44 | VVNKGHFDGQ | M60;M3;M27;M39 |
| 1685 | TLKATEETFKLSYGIA | M12;M6;M44; | KSEDAQGMDNLACE | M4;M1;M3;M45;M37;M4; |
| | TVREVLSDR | M33;M59 | DLKPVSEEVVE | M1;M3;M45;M37 |
| 1686 | ATEETFKLSYGIATVR | M12;M6;M44; | NNCYLATALLTLQQI | M4;M58;M3;M48;M51;M4; |
| | EVLSDRELH | M33;M59 | ELKFNPPALQ | M58;M3;M48;M51 |
| 1687 | KATEETFKLSYGIATV | M12;M6;M44; | VVQLTSQWLTNIFGT | M4;M31;M3;M45;M59;M4; |
| | REVLSDREL | M33;M59 | VYEKLKPVLD | M31;M3;M45;M59 |
| 1688 | LKATEETFKLSYGIAT | M12;M6;M44; | GGVVQLTSQWLTNIF | M4;M3;M45;M42;M59;M4; |
| | VREVLSDRE | M33;M59 | GTVYEKLKPV | M3;M45;M42;M59 |
| 1689 | EETGTLIVNSVLLFLAF | M73;M14; | YSGVVTTVMFLARGI | M12;M14;M29;M3;M20; |
| | VVFLLVTL | M7;M50;M44 | VFMCVEYCPI | M12;M14;M29;M3;M20 |
| 1690 | LNTLVKQLSSNFGAISS | M34;M6; | ITEEVGHTDLMAAYV | M14;M70;M51;M63;M44; |
| | VLNDILSR | M48;M45;M22 | DNSSLTIKKP | M14;M70;M51;M63;M44 |
| 1691 | ALNTLVKQLSSNFGAI | M34;M6; | KITEEVGHTDLMAAY | M14;M70;M51;M63;M44; |
| | SSVLNDILS | M48;M45;M22 | VDNSSLTIKK | M14;M70;M51;M63;M44 |
| 1692 | FSQILPDPSKPSKRSFIE | M30;M36; | GTLSYEQFKKGVQIPC | M16;M1;M3;M37;M52;M16; |
| | DLLFNKV | M63;M67;M22 | TCGKQATKY | M1;M3;M37;M52 |
| 1693 | NFSQILPDPSKPSKRSFI | M30;M36; | GGDAALALLLLDRLN | M56;M58;M16;M55;M10; |
| | EDLLFNK | M63;M67;M22 | QLESKMSGKG | M53;M51;M36;M17;M11 |
| 1694 | SQILPDPSKPSKRSFIED | M30;M36; | LAAIMQLFFSYFAVHF | M3;M48;M45;M9;M23;M3; |
| | LLFNKVT | M63;M67;M22 | ISNSWLMWL | M48;M45;M9;M23 |
| 1695 | QLSSNFGAISSVLNDIL | M34;M6; | AIMQLFFSYFAVHFIS | M3;M48;M45;M9;M23;M3; |
| | SRLDKVEA | M48;M45;M50 | NSWLMWLII | M48;M45;M9;M23 |
| 1696 | LVKQLSSNFGAISSVL | M34;M6; | GLAAIMQLFFSYFAV | M3;M48;M45;M9;M23;M3; |
| | NDILSRLDK | M48;M45;M50 | HFISNSWLMW | M48;M45;M9;M23 |
| 1697 | KQLSSNFGAISSVLNDI | M34;M6; | AAIMQLFFSYFAVHFI | M3;M48;M45;M9;M23;M3; |
| | LSRLDKVE | M48;M45;M50 | SNSWLMWLI | M48;M45;M9;M23 |
| 1698 | VKQLSSNFGAISSVLN | M34;M6; | ACVEEVTTTLEETKFL | M28;M3;M6;M50;M20;M28; |
| | DILSRLDKV | M48;M45;M50 | TENLLLYID | M3;M6;M50;M20 |
| 1699 | GPHEFCSQHTMLVKQ | M14;M29; | CVEEVTTTLEETKFLT | M28;M3;M6;M50;M20;M28; |
| | GDDYVYLPYP | M6;M18;M39 | ENLLLYIDI | M3;M6;M50;M20 |
| 1700 | KGPHEFCSQHTMLVK | M14;M29; | LLSDLQDLKWARFPK | M4;M77;M54;M60;M3;M4; |
| | QGDDYVYLPY | M6;M18;M39 | SDGTGTIYTE | M77;M54;M60;M3 |
| 1701 | SAQCFKMFYKGVITH | M70;M45; | ALLSDLQDLKWARFP | M4;M77;M54;M60;M3;M4; |
| | DVSSAINRPQ | M36;M52;M11 | KSDGTGTIYT | M77;M54;M60;M3 |
| 1702 | QNAVASKILGLPTQTV | M21;M54; | LALLSDLQDLKWARF | M4;M77;M54;M60;M3;M4; |
| | DSSQGSEYD | M60;M51;M11 | PKSDGTGTIY | M77;M54;M60;M3 |
| 1703 | NEVAKNLNESLIDLQE | M70;M61; | IKACVEEVTTTLEETK | M32;M28;M3;M6;M20;M32; |
| | LGKYEQYIK | M51;M67;M17 | FLTENLLLY | M28;M3;M6;M20 |
| 1704 | RLNEVAKNLNESLIDL | M70;M61; | KACVEEVTTTLEETK | M32;M28;M3;M6;M20;M32; |
| | QELGKYEQY | M51;M67;M17 | FLTENLLLYI | M28;M3;M6;M20 |
| 1705 | LNEVAKNLNESLIDLQ | M70;M61; | GDYILANTCTERLKLF | M56;M73;M19;M31;M6; |
| | ELGKYEQYI | M51;M67;M17 | AAETLKATE | M48;M44;M18;M63;M47 |
| 1706 | AKNLNESLIDLQELGK | M70;M61; | APHGVVFLHVTYVPA | M64;M21;M28;M66;M70; |
| | YEQYIKWPW | M51;M67;M17 | QEKNFTTAPA | M53;M3;M33;M26;M9 |
| 1707 | KNLNESLIDLQELGKY | M70;M61; | QSAPHGVVFLHVTYV | M64;M21;M28;M66;M70; |
| | EQYIKWPWY | M51;M67;M17 | PAQEKNFTTA | M53;M3;M33;M26;M9 |
| 1708 | VAKNLNESLIDLQELG | M70;M61; | SAPHGVVFLHVTYVP | M64;M21;M28;M66;M70; |
| | KYEQYIKWP | M51;M67;M17 | AQEKNFTTAP | M53;M3;M33;M26;M9 |
| 1709 | EVAKNLNESLIDLQEL | M70;M61; | FPQSAPHGVVFLHVT | M64;M21;M28;M66;M70; |
| | GKYEQYIKW | M51;M67;M17 | YVPAQEKNFT | M53;M3;M33;M26;M9 |
| 1710 | KMFYKGVITHDVSSAI | M21;M70; | PQSAPHGVVFLHVTY | M64;M21;M28;M66;M70; |
| | NRPQIGVVR | M45;M36;M11 | VPAQEKNFTT | M53;M3;M33;M26;M9 |
| 1711 | FYKGVITHDVSSAINR | M21;M70; | TGYKKPASRELKVTF | M21;M70;M3;M57;M63; |
| | PQIGVVREF | M45;M36;M11 | FPDLNGDVVA | M21;M70;M3;M57;M63 |
| 1712 | FKMFYKGVITHDVSSA | M21;M70; | GYKKPASRELKVTFF | M21;M70;M3;M57;M63; |
| | INRPQIGVV | M45;M36;M11 | PDLNGDVVAI | M21;M70;M3;M57;M63 |
| 1713 | CFKMFYKGVITHDVSS | M21;M70; | IVTALRANSAVKLQN | M73;M51;M39;M11;M44; |
| | AINRPQIGV | M45;M36;M11 | NELSPVALRQ | M73;M51;M39;M11;M44 |
| 1714 | QCFKMFYKGVITHDV | M21;M70; | LIVTALRANSAVKLQ | M73;M51;M39;M11;M44; |
| | SSAINRPQIG | M45;M36;M11 | NNELSPVALR | M73;M51;M39;M11;M44 |
| 1715 | MFYKGVITHDVSSAIN | M21;M70; | PRVFSAVGNICYTPSK | M49;M3;M43;M8;M42;M49; |
| | RPQIGVVRE | M45;M36;M11 | LIEYTDFAT | M3;M43;M8;M42 |
| 1716 | DRLNEVAKNLNESLID | M70;M61; | GDYFVLTSHTVMPLS | M54;M55;M60;M34;M29; |
| | LQELGKYEQ | M79;M51;M67 | APTLVPQEHY | M45;M51;M42;M46;M39 |
| 1717 | PARMAGNGGDAALAL | M13;M58; | IPRRNVATLQAENVT | M28;M54;M29;M6;M45; |
| | LLLDRLNQLE | M53;M51;M11 | GLFKDCSKVI | M38;M36;M27;M59;M20 |
| 1718 | TPSDFVRATATIPIQAS | M14;M34; | GKTFYVLPNDDTLRV | M3;M74;M43;M39;M22;M3; |
| | LPFGWLIV | M29;M45;M33 | EAFEYYHTTD | M74;M43;M39;M22 |
| 1719 | CTFEYVSQPFLMDLEG | M1;M57; | YCFLGYFCTCYFGLF | M56;M60;M3;M6;M18;M56; |
| | KQGNFKNLR | M36;M46;M20 | CLLNRYFRLT | M60;M3;M6;M18 |
| 1720 | ANQVIVNNLDKSAGFP | M58;M50; | CFLGYFCTCYFGLFCL | M56;M60;M3;M6;M18;M56; |
| | FNKWGKARL | M33;M51;M17 | LNRYFRLTL | M60;M3;M6;M18 |
| 1721 | INANQVIVNNLDKSAG | M58;M50; | RRCPAEIVDTVSALV | M12;M16;M7;M29;M6;M38; |
| | FPFNKWGKA | M33;M51;M17 | YDNKLKAHKD | M8;M17;M59;M23 |
| 1722 | GCINANQVIVNNLDKS | M58;M50; | WTNAGDYILANTCTE | M56;M73;M19;M31;M6; |
| | AGFPFNKWG | M33;M51;M17 | RLKLFAAETL | M44;M18;M8;M52;M47 |
| 1723 | GGCINANQVIVNNLDK | M58;M50; | TCDWTNAGDYILANT | M56;M73;M19;M31;M6; |
| | SAGFPFNKW | M33;M51;M17 | CTERLKLFAA | M44;M18;M8;M52;M47 |
| 1724 | NANQVIVNNLDKSAG | M58;M50; | DWTNAGDYILANTCT | M56;M73;M19;M31;M6; |
| | FPFNKWGKAR | M33;M51;M17 | ERLKLFAAET | M44;M18;M8;M52;M47 |
| 1725 | CINANQVIVNNLDKSA | M58;M50; | CDWTNAGDYILANTC | M56;M73;M19;M31;M6; |
| | GFPFNKWGK | M33;M51;M17 | TERLKLFAAE | M44;M18;M8;M52;M47 |
| 1726 | PRRNVATLQAENVTG | M29;M38; | VEKGIYQTSNFRVQPT | M31;M34;M30;M37;M50; |
| | LFKDCSKVIT | M45;M36;M59 | ESIVRFPNI | M51;M41;M8;M39;M23 |
| 1727 | WDYKRDAPAHISTIGV | M70;M6; | FYLTNDVSFLAHIQW | M54;M58;M60;M30;M3; |
| | CSMTDIAKK | M36;M11;M59 | MVMFTPLVPF | M54;M58;M60;M30;M3 |
| 1728 | MEVTPSGTWLTYTGAI | M19;M41; | LTNDVSFLAHIQWMV | M54;M58;M60;M30;M3; |
| | KLDDKDPNF | M8;M35;M11 | MFTPLVPFWI | M54;M58;M60;M30;M3 |
| 1729 | DMYSVMLTNDNTSRY | M64;M7; | TNDVSFLAHIQWMV | M54;M58;M60;M30;M3; |
| | WEPEFYEAMY | M34;M36;M22 | MFTPLVPFWIT | M54;M58;M60;M30;M3 |
| 1730 | LDMYSVMLTNDNTSR | M64;M7; | YLTNDVSFLAHIQWM | M54;M58;M60;M30;M3; |
| | YWEPEFYEAM | M34;M36;M22 | VMFTPLVPFW | M54;M58;M60;M30;M3 |
| 1731 | IGMEVTPSGTWLTYTG | M19;M41; | TGYRVTKNSKVQIGE | M40;M14;M31;M24;M41; |
| | AIKLDDKDP | M8;M11;M23 | YTFEKGDYGD | M8;M43;M42;M39;M44 |
| 1732 | DKSAQCFKMFYKGVI | M13;M70; | AEIVDTVSALVYDNK | M12;M28;M16;M7;M38; |
| | THDVSSAINR | M45;M36;M52 | LKAHKDKSAQ | M50;M63;M17;M35;M59 |
| 1733 | PETTADIVVFDEISMAT | M55;M7; | IVDTVSALVYDNKLK | M12;M28;M16;M7;M38; |
| | NYDLSVVN | M48;M33;M49 | AHKDKSAQCF | M50;M63;M17;M35;M59 |
| 1734 | DVNLHSSRLSFKELLV | M38;M48; | EIVDTVSALVYDNKL | M12;M28;M16;M7;M38; |
| | YAADPAMHA | M57;M36;M20 | KAHKDKSAQC | M50;M63;M17;M35;M59 |
| 1735 | MPILTLTRALTAESHV | M12;M16; | DKLQFTSLEIPRRNVA | M28;M54;M29;M2;M6;M45; |
| | DTDLTKPYI | M58;M17;M8 | TLQAENVTG | M38;M27;M59;M20 |
| 1736 | VSQPFLMDLEGKQGN | M54;M6; | LQFTSLEIPRRNVATL | M28;M54;M60;M29;M2;M6; |
| | FKNLREFVFK | M36;M46;M59 | QAENVTGLF | M45;M27;M59;M20 |
| 1737 | KRISNCVADYSVLYNS | M73;M7; | YFAVHFISNSWLMWL | M9;M73;M3;M11;M23;M9; |
| | ASFSTFKCY | M15;M11;M44 | IINLVQMAPI | M73;M3;M11;M23 |
| 1738 | RISNCVADYSVLYNSA | M73;M7; | SYFAVHFISNSWLMW | M9;M73;M3;M11;M23;M9; |
| | SFSTFKCYG | M15;M11;M44 | LIINLVQMAP | M73;M3;M11;M23 |
| 1739 | FIEDLLFNKVTLADAG | M73;M48; | EIDRLNEVAKNLNESL | M11;M70;M78;M65;M2; |
| | FIKQYGDCL | M44;M11;M47 | IDLQELGKY | M61;M79;M51;M67;M82 |
| 1740 | QGEIKDATPSDFVRAT | M34;M45; | YLTFYLTNDVSFLAHI | M54;M58;M60;M3;M50; |
| | ATIPIQASL | M33;M8;M11 | QWMVMFTPL | M54;M58;M60;M3;M50 |
| 1741 | TETAHSCNVNRFNVAI | M19;M25; | YLYLTFYLTNDVSFL | M54;M58;M60;M3;M50; |
| | TRAKVGILC | M6;M33;M59 | AHIQWMVMFT | M54;M58;M60;M3;M50 |
| 1742 | ECFDKFKVNSTLEQYV | M40;M6; | LTFYLTNDVSFLAHIQ | M54;M58;M60;M3;M50; |
| | FCTVNALPE | M50;M18;M44 | WMVMFTPLV | M54;M58;M60;M3;M50 |
| 1743 | QSLQTYVTQQLIRAAE | M64;M21; | IYLYLTFYLTNDVSFL | M54;M58;M60;M3;M50; |
| | IRASANLAA | M12;M14;M49 | AHIQWMVMF | M54;M58;M60;M3;M50 |
| 1744 | LNRALTGIAVEQDKNT | M28;M14; | VIYLYLTFYLTNDVSF | M54;M58;M60;M3;M50; |
| | QEVFAQVKQ | M70;M67;M20 | LAHIQWMVM | M54;M58;M60;M3;M50 |
| 1745 | QLNRALTGIAVEQDK | M28;M14; | LYLTFYLTNDVSFLA | M54;M58;M60;M3;M50; |
| | NTQEVFAQVK | M70;M67;M20 | HIQWMVMFTP | M54;M58;M60;M3;M50 |
| 1746 | PVTLACFVLAAVYRIN | M58;M70; | GRFVLALLSDLQDLK | M77;M54;M70;M60;M3; |
| | WITGGIAIA | M33;M63;M44 | WARFPKSDGT | M77;M54;M70;M60;M3 |
| 1747 | FLWLLWPVTLACFVL | M58;M14; | SSTCMMCYKRNRAT | M14;M8;M39;M11;M44; |
| | AAVYRINWIT | M70;M33;M63 | RVECTTIVNGV | M14;M8;M39;M11;M44 |
| 1748 | EDLLFNKVTLADAGFI | M73;M70; | NSSTCMMCYKRNRA | M14;M8;M39;M11;M44; |
| | KQYGDCLGD | M48;M41;M44 | TRVECTTIVNG | M14;M8;M39;M11;M44 |
| 1749 | PRQKRTATKAYNVTQ | M19;M70; | NVSLVKPSFYVYSRV | M40;M71;M54;M12;M16; |
| | AFGRRGPEQT | M78;M25;M2 | KNLNSSRVPD | M60;M33;M8;M46;M39 |
| 1750 | KPRQKRTATKAYNVT | M19;M70; | VSLVKPSFYVYSRVK | M40;M71;M54;M12;M16; |
| | QAFGRRGPEQ | M78;M25;M2 | NLNSSRVPDL | M60;M33;M8;M46;M39 |
| 1751 | AFDIYNDKVAGFAKFL | M4;M55; | CMMCYKRNRATRVE | M14;M29;M8;M39;M44; |
| | KTNCCRFQE | M7;M78;M79 | CTTIVNGVRRS | M14;M29;M8;M39;M44 |
| 1752 | PLSETKCTLKSFTVEK | M75;M21; | STCMMCYKRNRATR | M14;M29;M8;M39;M44; |
| | GIYQTSNFR | M14;M25;M63 | VECTTIVNGVR | M14;M29;M8;M39;M44 |
| 1753 | DPLSETKCTLKSFTVE | M75;M21; | MMCYKRNRATRVEC | M14;M29;M8;M39;M44; |
| | KGIYQTSNF | M14;M25;M63 | TTIVNGVRRSF | M14;M29;M8;M39;M44 |
| 1754 | LSETKCTLKSFTVEKGI | M75;M21; | MCYKRNRATRVECTT | M14;M29;M8;M39;M44; |
| | YQTSNFRV | M14;M25;M63 | IVNGVRRSFY | M14;M29;M8;M39;M44 |
| 1755 | LDPLSETKCTLKSFTV | M75;M21; | TCMMCYKRNRATRV | M14;M29;M8;M39;M44; |
| | EKGIYQTSN | M14;M25;M63 | ECTTIVNGVRR | M14;M29;M8;M39;M44 |
| 1756 | FPNITNLCPFGEVFNAT | M28;M66; | EVPANSTVLSFCAFA | M28;M48;M36;M8;M49; |
| | RFASVYAW | M58;M45;M17 | VDAAKAYKDY | M28;M48;M36;M8;M49 |
| 1757 | KNLREFVFKNIDGYFK | M71;M19; | ADYNYKLPDDFTGCV | M13;M12;M14;M29;M38; |
| | IYSKHTPIN | M16;M15;M52 | IAWNSNNLDS | M48;M67;M68;M49;M26 |
| 1758 | NLREFVFKNIDGYFKI | M71;M19; | RMAGNGGDAALALL | M13;M56;M58;M16;M55; |
| | YSKHTPINL | M16;M15;M52 | LLDRLNQLESK | M10;M53;M51;M17;M11 |
| 1759 | FQFCNDPFLGVYYHK | M4;M21; | TIQRKYKGIKIQEGVV | M40;M14;M5;M51;M67; |
| | NNKSWMESEF | M1;M37;M22 | DYGARFYFY | M40;M14;M5;M51;M67 |
| 1760 | EFQFCNDPFLGVYYHK | M4;M21; | STIQRKYKGIKIQEGV | M40;M14;M5;M51;M67; |
| | NNKSWMESE | M1;M37;M22 | VDYGARFYF | M40;M14;M5;M51;M67 |
| 1761 | PQADVEWKFYDAQPC | M29;M6; | VVDGCNSSTCMMCY | M71;M55;M8;M39;M44; |
| | SDKAYKIEEL | M41;M8;M59 | KRNRATRVECT | M71;M55;M8;M39;M44 |
| 1762 | CTVNALPETTADIVVF | M28;M55; | HVVDGCNSSTCMMC | M71;M55;M8;M39;M44; |
| | DEISMATNY | M38;M48;M49 | YKRNRATRVEC | M71;M55;M8;M39;M44 |
| 1763 | TVNALPETTADIVVFD | M28;M55; | TSKETLYCIDGALLTK | M10;M36;M35;M39;M44; |
| | EISMATNYD | M38;M48;M49 | SSEYKGPIT | M10;M36;M35;M39;M44 |
| 1764 | FCTVNALPETTADIVV | M28;M55; | VPAQEKNFTTAPAICH | M24;M78;M65;M7;M29;M2; |
| | FDEISMATN | M38;M48;M49 | DGKAHFPRE | M45;M79;M33;M82 |
| 1765 | GPEAGLPYGANKDGII | M64;M14; | YVPAQEKNFTTAPAIC | M24;M78;M65;M7;M29;M2; |
| | WVATEGALN | M30;M57;M22 | HDGKAHFPR | M45;M79;M33;M82 |
| 1766 | EAGLPYGANKDGIIWV | M64;M14; | GCNSSTCMMCYKRN | M71;M8;M39;M11;M44; |
| | ATEGALNTP | M30;M57;M22 | RATRVECTTIV | M71;M8;M39;M11;M44 |
| 1767 | PEAGLPYGANKDGIIW | M64;M14; | LLPSLATVAYFNMVY | M28;M6;M63;M46;M39; |
| | VATEGALNT | M30;M57;M22 | MPASWVMRIM | M28;M6;M63;M46;M39 |
| 1768 | FLGTCRRCPAEIVDTV | M29;M38; | LFLLPSLATVAYFNM | M28;M6;M63;M46;M39; |
| | SALVYDNKL | M6;M8;M23 | VYMPASWVMR | M28;M6;M63;M46;M39 |
| 1769 | MFLGTCRRCPAEIVDT | M29;M38; | FLLPSLATVAYFNMV | M28;M6;M63;M46;M39; |
| | VSALVYDNK | M6;M8;M23 | YMPASWVMRI | M28;M6;M63;M46;M39 |
| 1770 | KIADKYVRNLQHRLY | M54;M60; | YYKKDNSYFTEQPID | M64;M55;M60;M51;M39; |
| | ECLYRNRDVD | M2;M37;M44 | LVPNQPYPNA | M64;M55;M60;M51;M39 |
| 1771 | IADKYVRNLQHRLYE | M54;M60; | NYYKKDNSYFTEQPI | M64;M55;M60;M51;M39; |
| | CLYRNRDVDT | M2;M37;M44 | DLVPNQPYPN | M64;M55;M60;M51;M39 |
| 1772 | ETQALPQRQKKQQTV | M54;M14; | KKDNSYFTEQPIDLVP | M64;M55;M60;M51;M39; |
| | TLLPAADLDD | M29;M26;M39 | NQPYPNASF | M64;M55;M60;M51;M39 |
| 1773 | TQALPQRQKKQQTVT | M54;M14; | YKKDNSYFTEQPIDL | M64;M55;M60;M51;M39; |
| | LLPAADLDDF | M29;M26;M39 | VPNQPYPNAS | M64;M55;M60;M51;M39 |
| 1774 | TKRFDNPVLPFNDGVY | M64;M12; | LANTCTERLKLFAAE | M4;M64;M21;M40;M48; |
| | FASTEKSNI | M24;M28;M22 | TLKATEETFK | M63;M49;M26;M22;M23 |
| 1775 | KRFDNPVLPFNDGVYF | M64;M12; | ANTCTERLKLFAAET | M4;M64;M21;M40;M48; |
| | ASTEKSNII | M24;M28;M22 | LKATEETFKL | M63;M49;M26;M22;M23 |
| 1776 | NKDGIIWVATEGALNT | M64;M30; | NTCTERLKLFAAETL | M4;M64;M21;M40;M48; |
| | PKDHIGTRN | M6;M72;M22 | KATEETFKLS | M63;M49;M26;M22;M23 |
| 1777 | KDGIIWVATEGALNTP | M64;M30; | CYLATALLTLQQIELK | M73;M58;M48;M74;M51; |
| | KDHIGTRNP | M6;M72;M22 | FNPPALQDA | M73;M58;M48;M74;M51 |
| 1778 | FTTAPAICHDGKAHFP | M56;M24; | ATALLTLQQIELKFNP | M73;M58;M48;M74;M51; |
| | REGVFVSNG | M7;M45;M35 | PALQDAYYR | M73;M58;M48;M74;M51 |
| 1779 | TAPAICHDGKAHFPRE | M56;M24; | YLATALLTLQQIELKF | M73;M58;M48;M74;M51; |
| | GVFVSNGTH | M7;M45;M35 | NPPALQDAY | M73;M58;M48;M74;M51 |
| 1780 | NFTTAPAICHDGKAHF | M56;M24; | LATALLTLQQIELKFN | M73;M58;M48;M74;M51; |
| | PREGVFVSN | M7;M45;M35 | PPALQDAYY | M73;M58;M48;M74;M51 |
| 1781 | KNFTTAPAICHDGKAH | M56;M24; | NCYLATALLTLQQIEL | M73;M58;M48;M74;M51; |
| | FPREGVFVS | M7;M45;M35 | KFNPPALQD | M73;M58;M48;M74;M51 |
| 1782 | TTAPAICHDGKAHFPR | M56;M24; | ALLTLQQIELKFNPPA | M73;M58;M48;M74;M51; |
| | EGVFVSNGT | M7;M45;M35 | LQDAYYRAR | M73;M58;M48;M74;M51 |
| 1783 | EKNFTTAPAICHDGKA | M56;M24; | TALLTLQQIELKFNPP | M73;M58;M48;M74;M51; |
| | HFPREGVFV | M7;M45;M35 | ALQDAYYRA | M73;M58;M48;M74;M51 |
| 1784 | SPRWYFYYLGTGPEA | M32;M12; | SSYIVDSVTVKNGSIH | M21;M12;M48;M36;M49; |
| | GLPYGANKDG | M31;M6;M52 | LYFDKAGQK | M21;M12;M48;M36;M49 |
| 1785 | KRDAPAHISTIGVCSM | M66;M70; | HADQLTPTWRVYSTG | M75;M58;M14;M24;M16; |
| | TDIAKKPTE | M6;M63;M59 | SNVFQTRAGC | M5;M6;M41;M17;M82 |
| 1786 | RDAPAHISTIGVCSMT | M66;M70; | WNIGEQKSILSPLYAF | M6;M48;M45;M49;M22;M6; |
| | DIAKKPTET | M6;M63;M59 | ASEAARVVR | M48;M45;M49;M22 |
| 1787 | DAPAHISTIGVCSMTDI | M66;M70; | DPNFKDQVILLNKHID | M56;M73;M14;M53;M44; |
| | AKKPTETI | M6;M63;M59 | AYKTFPPTE | M17;M35;M52;M11;M47 |
| 1788 | APAHISTIGVCSMTDIA | M66;M70; | KDPNFKDQVILLNKHI | M56;M73;M14;M53;M44; |
| | KKPTETIC | M6;M63;M59 | DAYKTFPPT | M17;M35;M52;M11;M47 |
| 1789 | GTKRFDNPVLPFNDGV | M64;M12; | QIRSAAKKNNLPFKLT | M19;M12;M78;M33;M8; |
| | YFASTEKSN | M28;M82;M22 | CATTRQVVN | M19;M12;M78;M33;M8 |
| 1790 | RTTCFSVAALTNNVAF | M73;M1; | KDQVILLNKHIDAYK | M56;M73;M14;M53;M44; |
| | QTVKPGNFN | M6;M22;M59 | TFPPTEPKKD | M41;M35;M52;M11;M47 |
| 1791 | LTVLPPLLTDEMIAQY | M7;M38; | NGVSFSTFEEAALCTF | M4;M13;M6;M49;M44;M4; |
| | TSALLAGTI | M48;M41;M49 | LLNKEMYLK | M13;M6;M49;M44 |
| 1792 | LEAPFLYLYALVYFLQ | M4;M54; | NQDLNGNWYDFGDFI | M4;M40;M9;M58;M16;M70; |
| | SINFVRIIM | M12;M60;M25 | QTTPGSGVPV | M34;M8;M15;M11 |
| 1793 | GLEAPFLYLYALVYFL | M4;M54; | QDLNGNWYDFGDFIQ | M4;M40;M9;M58;M16;M70; |
| | QSINFVRII | M12;M60;M25 | TTPGSGVPVV | M34;M8;M15;M11 |
| 1794 | EAPFLYLYALVYFLQS | M4;M54; | ANSTVLSFCAFAVDA | M32;M12;M28;M36;M69; |
| | INFVRIIMR | M12;M60;M25 | AKAYKDYLAS | M32;M12;M28;M36;M69 |
| 1795 | TVYDPLQPELDSFKEE | M70;M34; | AQGMDNLACEDLKP | M4;M1;M45;M37;M49;M4; |
| | LDKYFKNHT | M6;M63;M59 | VSEEVVENPTI | M1;M45;M37;M49 |
| 1796 | VVRIKIVQMLSDTLKN | M61;M57; | SDDYIATNGPLKVGG | M14;M10;M29;M11;M44; |
| | LSDRVVFVL | M36;M46;M20 | SCVLSGHNLA | M14;M10;M29;M11;M44 |
| 1797 | LPWNVVRIKIVQMLSD | M61;M57; | DITFLKKDAPYIVGDV | M14;M7;M30;M57;M11; |
| | TLKNLSDRV | M36;M46;M20 | VQEGVLTAV | M14;M7;M30;M57;M11 |
| 1798 | NVVRIKIVQMLSDTLK | M61;M57; | TSNFRVQPTESIVRFP | M21;M31;M34;M30;M48; |
| | NLSDRVVFV | M36;M46;M20 | NITNLCPFG | M50;M41;M26;M39;M22 |
| 1799 | WNVVRIKIVQMLSDTL | M61;M57; | SVYYTSNPTTFHLDG | M7;M48;M45;M50;M49;M7; |
| | KNLSDRVVF | M36;M46;M20 | EVITFDNLKT | M48;M45;M50;M49 |
| 1800 | PWNVVRIKIVQMLSDT | M61;M57; | REVRTIKVFTTVDNIN | M29;M51;M43;M42;M26; |
| | LKNLSDRVV | M36;M46;M20 | LHTQVVDMS | M29;M51;M43;M42;M26 |
| 1801 | TVREVLSDRELHLSWE | M28;M70; | YTVELGTEVNEFACV | M14;M66;M34;M8;M39; |
| | VGKPRPPLN | M5;M6;M59 | VADAVIKTLQ | M14;M66;M34;M8;M39 |
| 1802 | ATVREVLSDRELHLS | M28;M70; | DSVTVKNGSIHLYFD | M21;M12;M7;M48;M49; |
| | WEVGKPRPPL | M5;M6;M59 | KAGQKTYERH | M21;M12;M7;M48;M49 |
| 1803 | VREVLSDRELHLSWE | M28;M70; | LDYIINLIIKNLSKSLT | M56;M73;M28;M58;M50; |
| | VGKPRPPLNR | M5;M6;M59 | ENKYSQLD | M18;M17;M15;M11;M44 |
| 1804 | VYDPLQPELDSFKEEL | M75;M70; | NLDYIINLIIKNLSKSL | M56;M73;M28;M58;M50; |
| | DKYFKNHTS | M6;M63;M59 | TENKYSQL | M18;M17;M15;M11;M44 |
| 1805 | PLQPELDSFKEELDKY | M75;M70; | YAFASEAARVVRSIFS | M4;M14;M48;M42;M59;M4; |
| | FKNHTSPDV | M6;M63;M59 | RTLETAQNS | M14;M48;M42;M59 |
| 1806 | DPLQPELDSFKEELDK | M75;M70; | GTEVNEFACVVADAV | M14;M25;M34;M8;M39; |
| | YFKNHTSPD | M6;M63;M59 | IKTLQPVSEL | M14;M25;M34;M8;M39 |
| 1807 | YDPLQPELDSFKEELD | M75;M70; | LLLQLCTFTRSTNSRI | M14;M33;M8;M42;M39; |
| | KYFKNHTSP | M6;M63;M59 | KASMPTTIA | M14;M33;M8;M42;M39 |
| 1808 | PSKRSFIEDLLFNKVTL | M73;M44; | LTTAAKLMVVIPDYN | M57;M51;M41;M82;M23; |
| | ADAGFIKQ | M67;M11;M47 | TYKNTCDGTT | M57;M51;M41;M82;M23 |
| 1809 | FLVFLGIITTVAAFHQE | M30;M29; | TTAAKLMVVIPDYNT | M57;M51;M41;M82;M23; |
| | CSLQSCTQ | M6;M22;M59 | YKNTCDGTTF | M57;M51;M41;M82;M23 |
| 1810 | LVFLGIITTVAAFHQEC | M30;M29; | RDGCVPLNIIPLTTAA | M19;M24;M14;M38;M22; |
| | SLQSCTQH | M6;M22;M59 | KLMVVIPDY | M19;M24;M14;M38;M22 |
| 1811 | PKEITVATSRTLSYYK | M70;M48; | DGCVPLNIIPLTTAAK | M19;M24;M14;M38;M22; |
| | LGASQRVAG | M63;M49;M23 | LMVVIPDYN | M19;M24;M14;M38;M22 |
| 1812 | ESEFRVYSSANNCTFE | M64;M5;M2 | PVPYCYDTNVLEGSV | M12;M14;M5;M29;M46; |
| | YVSQPFLMD | M48;M82 | AYESLRPDTR | M12;M14;M5;M29;M46 |
| 1813 | QPTESIVRFPNITNLCP | M21;M28; | KPVPYCYDTNVLEGS | M12;M14;M5;M29;M46; |
| | FGEVFNAT | M66;M48;M41 | VAYESLRPDT | M12;M14;M5;M29;M46 |
| 1814 | DELTGHMLDMYSVML | M64;M31; | NNDYYRSLPGVFCGV | M25;M45;M8;M43;M39; |
| | TNDNTSRYWE | M34;M36;M22 | DAVNLLTNMF | M25;M45;M8;M43;M39 |
| 1815 | RNLQHRLYECLYRNR | M2;M6; | YRSLPGVFCGVDAVN | M25;M45;M8;M43;M39; |
| | DVDTDFVNEF | M35;M52;M47 | LLTNMFTPLI | M25;M45;M8;M43;M39 |
| 1816 | LPFFYYSDSPCESHGK | M71;M32; | YYRSLPGVFCGVDAV | M25;M45;M8;M43;M39; |
| | QVVSDIDYV | M55;M45;M41 | NLLTNMFTPL | M25;M45;M8;M43;M39 |
| 1817 | QLPFFYYSDSPCESHG | M71;M32; | DYYRSLPGVFCGVDA | M25;M45;M8;M43;M39; |
| | KQVVSDIDY | M55;M45;M41 | VNLLTNMFTP | M25;M45;M8;M43;M39 |
| 1818 | KQLPFFYYSDSPCESH | M71;M32; | NDYYRSLPGVFCGVD | M25;M45;M8;M43;M39; |
| | GKQVVSDID | M55;M45;M41 | AVNLLTNMFT | M25;M45;M8;M43;M39 |
| 1819 | SELVIGAVILRGHLRIA | M58;M14; | RSLPGVFCGVDAVNL | M25;M45;M8;M43;M39; |
| | GHHLGRCD | M55;M53;M17 | LTNMFTPLIQ | M25;M45;M8;M43;M39 |
| 1820 | ESELVIGAVILRGHLRI | M58;M14; | PASRELKVTFFPDLNG | M21;M70;M57;M36;M63; |
| | AGHHLGRC | M55;M53;M17 | DVVAIDYKH | M21;M70;M57;M36;M63 |
| 1821 | NQDVNLHSSRLSFKEL | M53;M38; | PAQEKNFTTAPAICHD | M56;M24;M78;M65;M7; |
| | LVYAADPAM | M57;M36;M20 | GKAHFPREG | M45;M79;M33;M35;M82 |
| 1822 | VDRYPANSIVCRFDTR | M5;M2; | PLNSIIKTIQPRVEKKK | M28;M29;M6;M72;M59; |
| | VLSNLNLPG | M48;M50;M9 | LDGFMGRI | M28;M29;M6;M72;M59 |
| 1823 | PAWRKAVFISPYNSQN | M21;M48; | FASEAARVVRSIFSRT | M4;M2;M48;M42;M59;M4; |
| | AVASKILGL | M49;M26;M39 | LETAQNSVR | M2;M48;M42;M59 |
| 1824 | AWRKAVFISPYNSQN | M21;M48; | ASEAARVVRSIFSRTL | M4;M2;M48;M42;M59;M4; |
| | AVASKILGLP | M49;M26;M39 | ETAQNSVRV | M2;M48;M42;M59 |
| 1825 | TRNPAWRKAVFISPYN | M21;M48; | TSNYSGVVTTVMFLA | M12;M14;M29;M38;M20; |
| | SQNAVASKI | M49;M26;M39 | RGIVFMCVEY | M12;M14;M29;M38;M20 |
| 1826 | RNPAWRKAVFISPYNS | M21;M48; | AKAYKDYLASGGQPI | M12;M37;M33;M8;M39; |
| | QNAVASKIL | M49;M26;M39 | TNCVKMLCTH | M12;M37;M33;M8;M39 |
| 1827 | NPAWRKAVFISPYNSQ | M21;M48; | KAYKDYLASGGQPIT | M12;M37;M33;M8;M39; |
| | NAVASKILG | M49;M26;M39 | NCVKMLCTHT | M12;M37;M33;M8;M39 |
| 1828 | WRKAVFISPYNSQNA | M21;M48; | VNEFACVVADAVIKT | M28;M78;M45;M49;M39; |
| | VASKILGLPT | M49;M26;M39 | LQPVSELLTP | M28;M78;M45;M49;M39 |
| 1829 | TFKVSIWNLDYIINLIIK | M56;M50; | INITRFQTLLALHRSY | M21;M24;M55;M5;M10;M2; |
| | NLSKSLT | M18;M11;M44 | LTPGDSSSG | M48;M20;M9;M23 |
| 1830 | DSATLPKGIMMNVAK | M73;M58; | PYCYDTNVLEGSVAY | M12;M14;M5;M34;M46; |
| | YTQLCQYLNT | M78;M11;M44 | ESLRPDTRYV | M12;M14;M5;M34;M46 |
| 1831 | SATLPKGIMMNVAKY | M73;M58; | NLACEDLKPVSEEVV | M73;M45;M50;M49;M44; |
| | TQLCQYLNTL | M78;M11;M44 | ENPTIQKDVL | M73;M45;M50;M49;M44 |
| 1832 | DGNKIADKYVRNLQH | M54;M60; | EDLKPVSEEVVENPTI | M73;M45;M50;M49;M44; |
| | RLYECLYRNR | M37;M57;M44 | QKDVLECNV | M73;M45;M50;M49;M44 |
| 1833 | EISMATNYDLSVVNAR | M19;M25; | LACEDLKPVSEEVVE | M73;M45;M50;M49;M44; |
| | LRAKHYVYI | M50;M18;M26 | NPTIQKDVLE | M73;M45;M50;M49;M44 |
| 1834 | ISMATNYDLSVVNARL | M19;M25; | CEDLKPVSEEVVENP | M73;M45;M50;M49;M44; |
| | RAKHYVYIG | M50;M18;M26 | TIQKDVLECN | M73;M45;M50;M49;M44 |
| 1835 | ASVYAWNRKRISNCV | M55;M7; | ACEDLKPVSEEVVEN | M73;M45;M50;M49;M44; |
| | ADYSVLYNSA | M74;M67;M27 | PTIQKDVLEC | M73;M45;M50;M49;M44 |
| 1836 | AQALNTLVKQLSSNFG | M53;M45; | KQIVESCGNFKVTKG | M19;M25;M41;M8;M23; |
| | AISSVLNDI | M15;M46;M22 | KAKKGAWNIG | M19;M25;M41;M8;M23 |
| 1837 | YNDKVAGFAKFLKTN | M73;M78; | AFKQIVESCGNFKVT | M19;M25;M41;M8;M23; |
| | CCRFQEKDED | M55;M79;M8 | KGKAKKGAWN | M19;M25;M41;M8;M23 |
| 1838 | NYDLSVVNARLRAKH | M1;M25; | FKQIVESCGNFKVTK | M19;M25;M41;M8;M23; |
| | YVYIGDPAQL | M37;M57;M26 | GKAKKGAWNI | M19;M25;M41;M8;M23 |
| 1839 | TNYDLSVVNARLRAK | M1;M25; | GVLMSNLGMPSYCTG | M73;M45;M18;M49;M44; |
| | HYVYIGDPAQ | M37;M57;M26 | YREGYLNSTN | M73;M45;M18;M49;M44 |
| 1840 | YDLSVVNARLRAKHY | M1;M25; | ASGKPVPYCYDTNVL | M30;M29;M36;M35;M46; |
| | VYIGDPAQLP | M37;M57;M26 | EGSVAYESLR | M30;M29;M36;M35;M46 |
| 1841 | GKIADYNYKLPDDFTG | M13;M12; | EAELAKNVSLDNVLS | M14;M7;M29;M61;M27; |
| | CVIAWNSNN | M29;M38;M67 | TFISAARQGF | M14;M7;M29;M61;M27 |
| 1842 | KIADYNYKLPDDFTGC | M13;M12; | AEAELAKNVSLDNVL | M14;M7;M29;M61;M27; |
| | VIAWNSNNL | M29;M38;M67 | STFISAARQG | M14;M7;M29;M61;M27 |
| 1843 | VVYRAFDIYNDKVAG | M78;M7; | AELAKNVSLDNVLST | M14;M7;M29;M61;M27; |
| | FAKFLKTNCC | M55;M79;M41 | FISAARQGFV | M14;M7;M29;M61;M27 |
| 1844 | TDVVYRAFDIYNDKV | M78;M7; | YPNMFITREEAIRHVR | M64;M32;M14;M34;M57; |
| | AGFAKFLKTN | M55;M79;M41 | AWIGFDVEG | M46;M69;M42;M39;M20 |
| 1845 | VYRAFDIYNDKVAGF | M78;M7; | QGLVASIKNFKSVLY | M21;M54;M55;M60;M48; |
| | AKFLKTNCCR | M55;M79;M41 | YQNNVFMSEA | M41;M49;M26;M22;M23 |
| 1846 | DVVYRAFDIYNDKVA | M78;M7; | VNTFSSTFNVPMEKL | M28;M70;M29;M8;M22; |
| | GFAKFLKTNC | M55;M79;M41 | KTLVATAEAE | M28;M70;M29;M8;M22 |
| 1847 | MKFLVFLGIITTVAAF | M64;M29; | LPFFSNVTWFHAIHVS | M64;M40;M29;M45;M49; |
| | HQECSLQSC | M6;M22;M59 | GTNGTKRFD | M15;M46;M26;M22;M23 |
| 1848 | SVVNARLRAKHYVYI | M40;M31; | FLPFFSNVTWFHAIHV | M64;M40;M29;M45;M49; |
| | GDPAQLPAPR | M1;M37;M57 | SGTNGTKRF | M15;M46;M26;M22;M23 |
| 1849 | DLSVVNARLRAKHYV | M31;M1; | LFLPFFSNVTWFHAIH | M64;M40;M29;M45;M49; |
| | YIGDPAQLPA | M37;M57;M26 | VSGTNGTKR | M15;M46;M26;M22;M23 |
| 1850 | RIVYTACSHAAVDALC | M28;M60; | FSSTFNVPMEKLKTL | M28;M81;M29;M6;M22; |
| | EKALKYLPI | M29;M67;M49 | VATAEAELAK | M28;M81;M29;M6;M22 |
| 1851 | IVYTACSHAAVDALCE | M28;M60; | FTPFDVVRQCSGVTF | M4;M54;M1;M60;M46;M4; |
| | KALKYLPID | M29;M67;M49 | QSAVKRTIKG | M54;M1;M60;M46 |
| 1852 | LVASIKNFKSVLYYQN | M21;M48; | EFTPFDVVRQCSGVTF | M4;M54;M1;M60;M46;M4; |
| | NVFMSEAKC | M41;M49;M22 | QSAVKRTIK | M54;M1;M60;M46 |
| 1853 | GLVASIKNFKSVLYYQ | M21;M48; | QKEKVNINIVGDFKL | M73;M7;M27;M35;M44; |
| | NNVFMSEAK | M41;M49;M22 | NEEIAIILAS | M73;M7;M27;M35;M44 |
| 1854 | DIYNDKVAGFAKFLKT | M4;M73; | ISASIVAGGIVAIVVTC | M21;M12;M60;M6;M22; |
| | NCCRFQEKD | M55;M78;M79 | LAYYFMRF | M21;M12;M60;M6;M22 |
| 1855 | MRTFKVSIWNLDYIIN | M56;M50; | APYIVGDVVQEGVLT | M14;M30;M29;M38;M67; |
| | LIIKNLSKS | M18;M44;M20 | AVVIPTKKAG | M14;M30;M29;M38;M67 |
| 1856 | RTFKVSIWNLDYIINLII | M56;M50; | KDAPYIVGDVVQEGV | M14;M30;M29;M38;M67; |
| | KNLSKSL | M18;M44;M20 | LTAVVIPTKK | M14;M30;M29;M38;M67 |
| 1857 | QKKQQTVTLLPAADL | M54;M55; | DAPYIVGDVVQEGVL | M14;M30;M29;M38;M67; |
| | DDFSKQLQQS | M46;M26;M39 | TAVVIPTKKA | M14;M30;M29;M38;M67 |
| 1858 | SRIIPARARVECFDKFK | M10;M50; | VEAFEYYHTTDPSFL | M1;M29;M6;M41;M59;M1; |
| | VNSTLEQY | M18;M9;M44 | GRYMSALNHT | M29;M6;M41;M59 |
| 1859 | IIPARARVECFDKFKV | M10;M50; | SVAYESLRPDTRYVL | M24;M7;M34;M48;M50; |
| | NSTLEQYVF | M18;M9;M44 | MDGSIIQFPN | M24;M7;M34;M48;M50 |
| 1860 | RIIPARARVECFDKFK | M10;M50; | GMDNLACEDLKPVSE | M1;M45;M37;M50;M49;M1; |
| | VNSTLEQYV | M18;M9;M44 | EVVENPTIQK | M45;M37;M50;M49 |
| 1861 | VIPTITQMNLKYAISAK | M58;M14; | AAMQRKLEKMADQA | M2;M74;M57;M63;M82;M2; |
| | NRARTVAG | M18;M35;M82 | MTQMYKQARSE | M74;M57;M63;M82 |
| 1862 | NVIPTITQMNLKYAISA | M58;M14; | CTFTRSTNSRIKASMP | M14;M33;M42;M39;M11; |
| | KNRARTVA | M18;M35;M82 | TTIAKNTVK | M14;M33;M42;M39;M11 |
| 1863 | CFNSTYASQGLVASIK | M64;M48; | LCTFTRSTNSRIKASM | M14;M33;M42;M39;M11; |
| | NFKSVLYYQ | M49;M22;M23 | PTTIAKNTV | M14;M33;M42;M39;M11 |
| 1864 | FNSTYASQGLVASIKN | M64;M48; | VLYQPPQTSITSAVLQ | M14;M81;M1;M38;M62; |
| | FKSVLYYQN | M49;M22;M23 | SGFRKMAFP | M14;M81;M1;M38;M62 |
| 1865 | VAALTNNVAFQTVKP | M73;M25; | LYQPPQTSITSAVLQS | M14;M81;M1;M38;M62; |
| | GNFNKDFYDF | M29;M6;M59 | GFRKMAFPS | M14;M81;M1;M38;M62 |
| 1866 | AALTNNVAFQTVKPG | M73;M25; | DVLYQPPQTSITSAVL | M14;M81;M1;M38;M62; |
| | NFNKDFYDFA | M29;M6;M59 | QSGFRKMAF | M14;M81;M1;M38;M62 |
| 1867 | INLVRDLPQGFSALEPL | M31;M34; | YQPPQTSITSAVLQSG | M14;M81;M1;M38;M62; |
| | VDLPIGIN | M45;M52;M47 | FRKMAFPSG | M14;M81;M1;M38;M62 |
| 1868 | DAQPCSDKAYKIEELF | M73;M70; | QPPQTSITSAVLQSGF | M14;M81;M1;M38;M62; |
| | YSYATHSDK | M29;M6;M59 | RKMAFPSGK | M14;M81;M1;M38;M62 |
| 1869 | YAWNRKRISNCVADY | M7;M74; | SDVLYQPPQTSITSAV | M14;M81;M1;M38;M62; |
| | SVLYNSASFS | M67;M27;M15 | LQSGFRKMA | M14;M81;M1;M38;M62 |
| 1870 | AWNRKRISNCVADYS | M7;M74; | ATYYLFDESGEFKLA | M16;M36;M15;M52;M23; |
| | VLYNSASFST | M67;M27;M15 | SHMYCSFYPP | M16;M36;M15;M52;M23 |
| 1871 | WNRKRISNCVADYSV | M7;M74; | DKKIKACVEEVTTTL | M32;M28;M6;M67;M20; |
| | LYNSASFSTF | M67;M27;M15 | EETKFLTENL | M32;M28;M6;M67;M20 |
| 1872 | ANVNALLSTDGNKIA | M10;M57; | KKIKACVEEVTTTLEE | M32;M28;M6;M67;M20; |
| | DKYVRNLQHR | M27;M8;M20 | TKFLTENLL | M32;M28;M6;M67;M20 |
| 1873 | NVNALLSTDGNKIAD | M10;M57; | LNRVCTNYMPYFFTL | M4;M29;M6;M69;M20;M4; |
| | KYVRNLQHRL | M27;M8;M20 | LLQLCTFTRS | M29;M6;M69;M20 |
| 1874 | TANVNALLSTDGNKIA | M10;M57; | CLNRVCTNYMPYFFT | M4;M29;M6;M69;M20;M4; |
| | DKYVRNLQH | M27;M8;M20 | LLLQLCTFTR | M29;M6;M69;M20 |
| 1875 | VNALLSTDGNKIADK | M10;M57; | RCLNRVCTNYMPYFF | M4;M29;M6;M69;M20;M4; |
| | YVRNLQHRLY | M27;M8;M20 | TLLLQLCTFT | M29;M6;M69;M20 |
| 1876 | KSAFVNLKQLPFFYYS | M32;M54; | RLTLGVYDYLVSTQE | M31;M6;M43;M8;M42;M31; |
| | DSPCESHGK | M60;M45;M41 | FRYMNSQGLL | M6;M43;M8;M42 |
| 1877 | KQRRPQGLPNNTASW | M40;M28; | FRLTLGVYDYLVSTQ | M31;M6;M43;M8;M42;M31; |
| | FTALTQHGKE | M70;M29;M38 | EFRYMNSQGL | M6;M43;M8;M42 |
| 1878 | KRNVIPTITQMNLKYA | M58;M14; | YFRLTLGVYDYLVST | M31;M6;M43;M8;M42;M31; |
| | ISAKNRART | M18;M35;M59 | QEFRYMNSQG | M6;M43;M8;M42 |
| 1879 | DLPQGFSALEPLVDLPI | M31;M60; | SSGVTRELMRELNGG | M54;M14;M60;M30;M22; |
| | GINITRFQ | M45;M52;M47 | AYTRYVDNNF | M54;M14;M60;M30;M22 |
| 1880 | LPQGFSALEPLVDLPIG | M31;M60; | HSSGVTRELMRELNG | M54;M14;M60;M30;M22; |
| | INITRFQT | M45;M52;M47 | GAYTRYVDNN | M54;M14;M60;M30;M22 |
| 1881 | CGDSTECSNLLLQYGS | M13;M48; | SGVTRELMRELNGGA | M54;M14;M60;M30;M22; |
| | FCTQLNRAL | M41;M49;M46 | YTRYVDNNFC | M54;M14;M60;M30;M22 |
| 1882 | ICGDSTECSNLLLQYG | M13;M48; | FLNKVVSTTTNIVTRC | M4;M78;M1;M37;M23;M4; |
| | SFCTQLNRA | M41;M49;M46 | LNRVCTNYM | M78;M1;M37;M23 |
| 1883 | VTANVNALLSTDGNKI | M10;M57; | LNKVVSTTTNIVTRCL | M4;M78;M1;M37;M23;M4; |
| | ADKYVRNLQ | M8;M9;M20 | NRVCTNYMP | M78;M1;M37;M23 |
| 1884 | LPPLLTDEMIAQYTSA | M38;M48; | VVRQCSGVTFQSAVK | M71;M19;M25;M33;M26; |
| | LLAGTITSG | M41;M49;M26 | RTIKGTHHWL | M71;M19;M25;M33;M26 |
| 1885 | LLTDEMIAQYTSALLA | M38;M48; | PSARIVYTACSHAAV | M64;M21;M28;M5;M60; |
| | GTITSGWTF | M41;M49;M26 | DALCEKALKY | M29;M38;M67;M17;M49 |
| 1886 | PLLTDEMIAQYTSALL | M38;M48; | ESDDYIATNGPLKVG | M10;M29;M18;M11;M44; |
| | AGTITSGWT | M41;M49;M26 | GSCVLSGHNL | M10;M29;M18;M11;M44 |
| 1887 | PPLLTDEMIAQYTSAL | M38;M48; | TTLEETKFLTENLLLY | M70;M50;M36;M11;M20; |
| | LAGTITSGW | M41;M49;M26 | IDINGNLHP | M70;M50;M36;M11;M20 |
| 1888 | MWLSYFIASFRLFART | M64;M40; | TTTLEETKFLTENLLL | M70;M50;M36;M11;M20; |
| | RSMWSFNPE | M16;M30;M15 | YIDINGNLH | M70;M50;M36;M11;M20 |
| 1889 | YYHKNNKSWMESEFR | M70;M5; | ALKGGKIVNNWLKQ | M56;M12;M55;M57;M11; |
| | VYSSANNCTF | M48;M41;M82 | LIKVTLVFLFV | M56;M12;M55;M57;M11 |
| 1890 | IMMNVAKYTQLCQYL | M78;M34; | SNSFDVLKSEDAQGM | M64;M6;M46;M39;M22; |
| | NTLTLAVPYN | M48;M41;M49 | DNLACEDLKP | M64;M6;M46;M39;M22 |
| 1891 | GIMMNVAKYTQLCQY | M78;M34; | NSFDVLKSEDAQGMD | M64;M6;M46;M39;M22; |
| | LNTLTLAVPY | M48;M41;M49 | NLACEDLKPV | M64;M6;M46;M39;M22 |
| 1892 | MMNVAKYTQLCQYL | M78;M34; | VVTTKIALKGGKIVN | M12;M55;M57;M11;M20; |
| | NTLTLAVPYNM | M48;M41;M49 | NWLKQLIKVT | M12;M55;M57;M11;M20 |
| 1893 | PHGVVFLHVTYVPAQ | M28;M66; | VTTKIALKGGKIVNN | M12;M55;M57;M11;M20; |
| | EKNFTTAPAI | M70;M33;M26 | WLKQLIKVTL | M12;M55;M57;M11;M20 |
| 1894 | VTIAEILLIIMRTFKVSI | M1;M5; | SMDNSPNLAWPLIVT | M73;M60;M18;M39;M44; |
| | WNLDYII | M61;M37;M20 | ALRANSAVKL | M73;M60;M18;M39;M44 |
| 1895 | TIAEILLIIMRTFKVSIW | M1;M5; | ISMDNSPNLAWPLIVT | M73;M60;M18;M39;M44; |
| | NLDYIIN | M61;M37;M20 | ALRANSAVK | M73;M60;M18;M39;M44 |
| 1896 | FQVTIAEILLIIMRTFK | M1;M5; | LMTARTVYDDGARR | M5;M6;M72;M11;M20;M5; |
| | VSIWNLDY | M61;M37;M20 | VWTLMNVLTLV | M6;M72;M11;M20 |
| 1897 | IAEILLIIMRTFKVSIWN | M1;M5; | SFYVYANGGKGFCKL | M41;M42;M8;M43;M39; |
| | LDYIINL | M61;M37;M20 | HNWNCVNCDT | M41;M42;M8;M43;M39 |
| 1898 | QVTIAEILLIIMRTFKV | M1;M5; | SEAARVVRSIFSRTLE | M34;M2;M48;M42;M59; |
| | SIWNLDYI | M61;M37;M20 | TAQNSVRVL | M34;M2;M48;M42;M59 |
| 1899 | WQLALSKGVHFVCNL | M50;M18; | SSPDAVTAYNGYLTS | M24;M55;M48;M41;M35; |
| | LLLFVTVYSH | M8;M49;M23 | SSKTPEEHFI | M24;M55;M48;M41;M35 |
| 1900 | KRWQLALSKGVHFVC | M50;M18; | SPDAVTAYNGYLTSS | M24;M55;M48;M41;M35; |
| | NLLLLFVTVY | M8;M49;M23 | SKTPEEHFIE | M24;M55;M48;M41;M35 |
| 1901 | RWQLALSKGVHFVCN | M50;M18; | KLKDCVMYASAVVL | M75;M12;M33;M46;M39; |
| | LLLLFVTVYS | M8;M49;M23 | LILMTARTVYD | M75;M12;M33;M46;M39 |
| 1902 | QLALSKGVHFVCNLLL | M50;M18; | LLILMTARTVYDDGA | M32;M6;M72;M11;M59; |
| | LFVTVYSHL | M8;M49;M23 | RRVWTLMNVL | M32;M6;M72;M11;M59 |
| 1903 | ASFRLFARTRSMWSFN | M64;M40; | GIVAIVVTCLAYYFM | M71;M16;M6;M62;M59; |
| | PETNILLNV | M16;M30;M42 | RFRRAFGEYS | M71;M16;M6;M62;M59 |
| 1904 | NSVCRLMKTIGPDMFL | M5;M6; | IVAIVVTCLAYYFMRF | M71;M16;M6;M62;M59; |
| | GTCRRCPAE | M37;M17;M59 | RRAFGEYSH | M71;M16;M6;M62;M59 |
| 1905 | NGTITVEELKKLLEQW | M4;M58; | GGIVAIVVTCLAYYF | M71;M16;M6;M62;M59; |
| | NLVIGFLFL | M53;M17;M47 | MRFRRAFGEY | M71;M16;M6;M62;M59 |
| 1906 | LKLRGTAVMSLKEGQI | M54;M60; | LKVDTANPKTPKYKF | M40;M77;M14;M34;M29; |
| | NDMILSLLS | M6;M49;M59 | VRIQPGQTFS | M40;M77;M14;M34;M29 |
| 1907 | KLRGTAVMSLKEGQI | M54;M60; | KTTEVVGDIILKPANN | M19;M58;M34;M53;M26; |
| | NDMILSLLSK | M6;M49;M59 | SLKITEEVG | M19;M58;M34;M53;M26 |
| 1908 | LRGTAVMSLKEGQIN | M54;M60; | VKTTEVVGDIILKPAN | M19;M58;M34;M53;M26; |
| | DMILSLLSKG | M6;M49;M59 | NSLKITEEV | M19;M58;M34;M53;M26 |
| 1909 | RGTAVMSLKEGQIND | M54;M60; | KVTLVFLFVAAIFYLI | M32;M1;M34;M18;M39; |
| | MILSLLSKGR | M6;M49;M59 | TPVHVMSKH | M32;M1;M34;M18;M39 |
| 1910 | EEAIRHVRAWIGFDVE | M32;M48; | TNVLEGSVAYESLRP | M19;M34;M50;M18;M46; |
| | GCHATREAV | M57;M69;M20 | DTRYVLMDGS | M19;M34;M50;M18;M46 |
| 1911 | TKCTLKSFTVEKGIYQ | M75;M21; | LLSMQGAVDINKLCE | M54;M60;M45;M67;M49; |
| | TSNFRVQPT | M25;M8;M23 | EMLDNRATLQ | M54;M60;M45;M67;M49 |
| 1912 | ETKCTLKSFTVEKGIY | M75;M21; | SVLLSMQGAVDINKL | M54;M60;M45;M67;M49; |
| | QTSNFRVQP | M25;M8;M23 | CEEMLDNRAT | M54;M60;M45;M67;M49 |
| 1913 | VNIQKEIDRLNEVAKN | M78;M65; | LSVLLSMQGAVDINK | M54;M60;M45;M67;M49; |
| | LNESLIDLQ | M2;M79;M82 | LCEEMLDNRA | M54;M60;M45;M67;M49 |
| 1914 | VVNIQKEIDRLNEVAK | M78;M65; | VLLSMQGAVDINKLC | M54;M60;M45;M67;M49; |
| | NLNESLIDL | M2;M79;M82 | EEMLDNRATL | M54;M60;M45;M67;M49 |
| 1915 | KNIDGYFKIYSKHTPIN | M40;M71; | DCVMYASAVVLLILM | M75;M32;M12;M46;M39; |
| | LVRDLPQG | M16;M15;M52 | TARTVYDDGA | M75;M32;M12;M46;M39 |
| 1916 | TECSNLLLQYGSFCTQ | M13;M48; | KDCVMYASAVVLLIL | M75;M32;M12;M46;M39; |
| | LNRALTGIA | M41;M49;M43 | MTARTVYDDG | M75;M32;M12;M46;M39 |
| 1917 | LMDLEGKQGNFKNLR | M54;M6; | HNDILLAKDTTEAFE | M32;M58;M53;M36;M68; |
| | EFVFKNIDGY | M43;M42;M59 | KMVSLLSVLL | M32;M58;M53;M36;M68 |
| 1918 | LEGKQGNFKNLREFVF | M54;M6; | NDILLAKDTTEAFEK | M32;M58;M53;M36;M68; |
| | KNIDGYFKI | M43;M42;M59 | MVSLLSVLLS | M32;M58;M53;M36;M68 |
| 1919 | DLEGKQGNFKNLREF | M54;M6; | IDHPNPKGFCDLKGK | M31;M63;M43;M42;M39; |
| | VFKNIDGYFK | M43;M42;M59 | YVQIPTTCAN | M31;M63;M43;M42;M39 |
| 1920 | EGKQGNFKNLREFVF | M54;M6; | DHPNPKGFCDLKGKY | M31;M63;M43;M42;M39; |
| | KNIDGYFKIY | M43;M42;M59 | VQIPTTCAND | M31;M63;M43;M42;M39 |
| 1921 | FLMDLEGKQGNFKNL | M54;M6; | HPNPKGFCDLKGKYV | M31;M63;M43;M42;M39; |
| | REFVFKNIDG | M43;M42;M59 | QIPTTCANDP | M31;M63;M43;M42;M39 |
| 1922 | MDLEGKQGNFKNLRE | M54;M6; | HIDHPNPKGFCDLKG | M31;M63;M43;M42;M39; |
| | FVFKNIDGYF | M43;M42;M59 | KYVQIPTTCA | M31;M63;M43;M42;M39 |
| 1923 | DAGFIKQYGDCLGDIA | M21;M70; | DIEVTGDSCNNYMLT | M8;M15;M52;M11;M47;M8; |
| | ARDLICAQK | M48;M50;M27 | YNKVENMTPR | M15;M52;M11;M47 |
| 1924 | MKDLSPRWYFYYLGT | M32;M31; | VLSTFISAARQGFVDS | M16;M70;M33;M18;M63; |
| | GPEAGLPYGA | M6;M68;M52 | DVETKDVVE | M16;M70;M33;M18;M63 |
| 1925 | KDLSPRWYFYYLGTG | M32;M31; | NVLSTFISAARQGFVD | M16;M70;M33;M18;M63; |
| | PEAGLPYGAN | M6;M68;M52 | SDVETKDVV | M16;M70;M33;M18;M63 |
| 1926 | KMKDLSPRWYFYYLG | M32;M31; | AGGIVAIVVTCLAYY | M71;M6;M62;M22;M59; |
| | TGPEAGLPYG | M6;M68;M52 | FMRFRRAFGE | M71;M6;M62;M22;M59 |
| 1927 | GKMKDLSPRWYFYYL | M32;M31; | KLTCATTRQVVNVVT | M64;M40;M29;M2;M22; |
| | GTGPEAGLPY | M6;M68;M52 | TKIALKGGKI | M64;M40;M29;M2;M22 |
| 1928 | DLSPRWYFYYLGTGPE | M32;M31; | TCATTRQVVNVVTTK | M64;M40;M29;M2;M22; |
| | AGLPYGANK | M6;M68;M52 | IALKGGKIVN | M64;M40;M29;M2;M22 |
| 1929 | SEFRVYSSANNCTFEY | M64;M5; | ATTRQVVNVVTTKIA | M64;M40;M29;M2;M22; |
| | VSQPFLMDL | M48;M82;M20 | LKGGKIVNNW | M64;M40;M29;M2;M22 |
| 1930 | QSLENVAFNVVNKGH | M71;M24; | FKLTCATTRQVVNVV | M64;M40;M29;M2;M22; |
| | FDGQQGEVPV | M16;M27;M15 | TTKIALKGGK | M64;M40;M29;M2;M22 |
| 1931 | LQSLENVAFNVVNKG | M71;M24; | CATTRQVVNVVTTKI | M64;M40;M29;M2;M22; |
| | HFDGQQGEVP | M16;M27;M15 | ALKGGKIVNN | M64;M40;M29;M2;M22 |
| 1932 | SHRFYRLANECAQVLS | M28;M79; | LTCATTRQVVNVVTT | M64;M40;M29;M2;M22; |
| | EMVMCGGSL | M42;M43;M46 | KIALKGGKIV | M64;M40;M29;M2;M22 |
| 1933 | GCVIAWNSNNLDSKV | M32;M48; | VEKKKLDGFMGRIRS | M58;M29;M6;M68;M26; |
| | GGNYNYLYRL | M50;M68;M26 | VYPVASPNEC | M58;M29;M6;M68;M26 |
| 1934 | HEETIYNLLKDCPAVA | M40;M30; | EKKKLDGFMGRIRSV | M58;M29;M6;M68;M26; |
| | KHDFFKFRI | M41;M42;M11 | YPVASPNECN | M58;M29;M6;M68;M26 |
| 1935 | EETIYNLLKDCPAVAK | M40;M30; | KKLDGFMGRIRSVYP | M58;M29;M6;M68;M26; |
| | HDFFKFRID | M41;M42;M11 | VASPNECNQM | M58;M29;M6;M68;M26 |
| 1936 | SLVLARKHTTCCSLSH | M81;M5; | KKKLDGFMGRIRSVY | M58;M29;M6;M68;M26; |
| | RFYRLANEC | M60;M2;M79 | PVASPNECNQ | M58;M29;M6;M68;M26 |
| 1937 | LVLARKHTTCCSLSHR | M81;M5; | AQVAKSHNIALIWNV | M32;M1;M37;M72;M68; |
| | FYRLANECA | M60;M2;M79 | KDFMSLSEQL | M32;M1;M37;M72;M68 |
| 1938 | FFKFRIDGDMVPHISR | M71;M24; | NAQVAKSHNIALIWN | M32;M1;M37;M72;M68; |
| | QRLTKYTMA | M55;M10;M26 | VKDFMSLSEQ | M32;M1;M37;M72;M68 |
| 1939 | FKFRIDGDMVPHISRQ | M71;M24; | EYYHTTDPSFLGRYM | M66;M1;M25;M37;M33; |
| | RLTKYTMAD | M55;M10;M26 | SALNHTKKWK | M66;M1;M25;M37;M33 |
| 1940 | FRIDGDMVPHISRQRL | M71;M24; | DAVIKTLQPVSELLTP | M78;M45;M79;M49;M82; |
| | TKYTMADLV | M55;M10;M26 | LGIDLDEWS | M78;M45;M79;M49;M82 |
| 1941 | RIDGDMVPHISRQRLT | M71;M24; | ADAVIKTLQPVSELLT | M78;M45;M79;M49;M82; |
| | KYTMADLVY | M55;M10;M26 | PLGIDLDEW | M78;M45;M79;M49;M82 |
| 1942 | DFFKFRIDGDMVPHIS | M71;M24; | VADAVIKTLQPVSELL | M78;M45;M79;M49;M82; |
| | RQRLTKYTM | M55;M10;M26 | TPLGIDLDE | M78;M45;M79;M49;M82 |
| 1943 | KFRIDGDMVPHISRQR | M71;M24; | WEIQQVVDADSKIVQ | M64;M54;M7;M60;M22; |
| | LTKYTMADL | M55;M10;M26 | LSEISMDNSP | M64;M54;M7;M60;M22 |
| 1944 | RIMASLVLARKHTTCC | M81;M5; | KFKEGVEFLRDGWEI | M4;M74;M49;M22;M20;M4; |
| | SLSHRFYRL | M60;M2;M35 | VKFISTCACE | M74;M49;M22;M20 |
| 1945 | IMASLVLARKHTTCCS | M81;M5; | PSVEQRKQDDKKIKA | M28;M65;M60;M74;M67; |
| | LSHRFYRLA | M60;M2;M35 | CVEEVTTTLE | M28;M65;M60;M74;M67 |
| 1946 | LRIMASLVLARKHTTC | M81;M5; | HVMSKHTDFSSEIIGY | M53;M48;M41;M43;M26; |
| | CSLSHRFYR | M60;M2;M35 | KAIDGGVTR | M53;M48;M41;M43;M26 |
| 1947 | ASLVLARKHTTCCSLS | M81;M5; | MSKHTDFSSEIIGYKA | M53;M48;M41;M43;M26; |
| | HRFYRLANE | M60;M2;M35 | IDGGVTRDI | M53;M48;M41;M43;M26 |
| 1948 | MASLVLARKHTTCCS | M81;M5; | DFSSEIIGYKAIDGGV | M53;M48;M41;M43;M26; |
| | LSHRFYRLAN | M60;M2;M35 | TRDIASTDT | M53;M48;M41;M43;M26 |
| 1949 | YVRNLQHRLYECLYR | M2;M37; | TDFSSEIIGYKAIDGG | M53;M48;M41;M43;M26; |
| | NRDVDTDFVN | M35;M52;M47 | VTRDIASTD | M53;M48;M41;M43;M26 |
| 1950 | KYVRNLQHRLYECLY | M2;M37; | SKHTDFSSEIIGYKAID | M53;M48;M41;M43;M26; |
| | RNRDVDTDFV | M35;M52;M47 | GGVTRDIA | M53;M48;M41;M43;M26 |
| 1951 | DKYVRNLQHRLYECL | M2;M37; | VMSKHTDFSSEIIGYK | M53;M48;M41;M43;M26; |
| | YRNRDVDTDF | M35;M52;M47 | AIDGGVTRD | M53;M48;M41;M43;M26 |
| 1952 | CSNLLLQYGSFCTQLN | M13;M48; | FSSEIIGYKAIDGGVT | M53;M48;M41;M43;M26; |
| | RALTGIAVE | M41;M43;M20 | RDIASTDTC | M53;M48;M41;M43;M26 |
| 1953 | FLAFVVFLLVTLAILTA | M54;M60; | HTDFSSEIIGYKAIDG | M53;M48;M41;M43;M26; |
| | LRLCAYCC | M25;M46;M39 | GVTRDIAST | M53;M48;M41;M43;M26 |
| 1954 | LLFLAFVVFLLVTLAIL | M54;M60; | KHTDFSSEIIGYKAID | M53;M48;M41;M43;M26; |
| | TALRLCAY | M25;M46;M39 | GGVTRDIAS | M53;M48;M41;M43;M26 |
| 1955 | LFLAFVVFLLVTLAILT | M54;M60; | GSVAYESLRPDTRYV | M24;M7;M34;M50;M18; |
| | ALRLCAYC | M25;M46;M39 | LMDGSIIQFP | M24;M7;M34;M50;M18 |
| 1956 | NARLRAKHYVYIGDP | M40;M31; | EGSVAYESLRPDTRY | M24;M7;M34;M50;M18; |
| | AQLPAPRTLL | M30;M37;M57 | VLMDGSIIQF | M24;M7;M34;M50;M18 |
| 1957 | VIRGDEVRQIAPGQTG | M80;M38; | CYFGLFCLLNRYFRLT | M56;M14;M60;M37;M18; |
| | KIADYNYKL | M33;M67;M62 | LGVYDYLVS | M56;M14;M60;M37;M18 |
| 1958 | RGDEVRQIAPGQTGKI | M80;M38; | FCTCYFGLFCLLNRYF | M56;M14;M60;M37;M18; |
| | ADYNYKLPD | M33;M67;M62 | RLTLGVYDY | M56;M14;M60;M37;M18 |
| 1959 | FVIRGDEVRQIAPGQT | M80;M38; | YFGLFCLLNRYFRLTL | M56;M14;M60;M37;M18; |
| | GKIADYNYK | M33;M67;M62 | GVYDYLVST | M56;M14;M60;M37;M18 |
| 1960 | IRGDEVRQIAPGQTGKI | M80;M38; | FGLFCLLNRYFRLTLG | M56;M14;M60;M37;M18; |
| | ADYNYKLP | M33;M67;M62 | VYDYLVSTQ | M56;M14;M60;M37;M18 |
| 1961 | TLLPAADLDDFSKQLQ | M71;M32; | TCYFGLFCLLNRYFRL | M56;M14;M60;M37;M18; |
| | QSMSSADST | M58;M55;M53 | TLGVYDYLV | M56;M14;M60;M37;M18 |
| 1962 | LLPAADLDDFSKQLQQ | M71;M32; | CTCYFGLFCLLNRYF | M56;M14;M60;M37;M18; |
| | SMSSADSTQ | M58;M55;M53 | RLTLGVYDYL | M56;M14;M60;M37;M18 |
| 1963 | VTLLPAADLDDFSKQL | M71;M32; | ARRVWTLMNVLTLV | M24;M1;M37;M41;M20; |
| | QQSMSSADS | M58;M55;M53 | YKVYYGNALDQ | M24;M1;M37;M41;M20 |
| 1964 | RLITGRLQSLQTYVTQ | M34;M29; | SVIYLYLTFYLTNDVS | M54;M58;M60;M48;M50; |
| | QLIRAAEIR | M49;M46;M22 | FLAHIQWMV | M54;M58;M60;M48;M50 |
| 1965 | LITGRLQSLQTYVTQQ | M34;M29; | YSVIYLYLTFYLTNDV | M54;M58;M60;M48;M50; |
| | LIRAAEIRA | M49;M46;M22 | SFLAHIQWM | M54;M58;M60;M48;M50 |
| 1966 | DRLITGRLQSLQTYVT | M34;M29; | KGSLPINVIVFDGKSK | M56;M7;M50;M52;M26; |
| | QQLIRAAEI | M49;M46;M22 | CEESSAKSA | M56;M7;M50;M52;M26 |
| 1967 | DFVNEFYAYLRKHFS | M71;M57; | GSLPINVIVFDGKSKC | M56;M7;M50;M52;M26; |
| | MMILSDDAVV | M18;M15;M35 | EESSAKSAS | M56;M7;M50;M52;M26 |
| 1968 | YDSMSYEDQDALFAY | M71;M55; | NTKGSLPINVIVFDGK | M56;M7;M50;M52;M26; |
| | TKRNVIPTIT | M10;M53;M9 | SKCEESSAK | M56;M7;M50;M52;M26 |
| 1969 | NSAIGKIQDSLSSTASA | M40;M64; | TKGSLPINVIVFDGKS | M56;M7;M50;M52;M26; |
| | LGKLQDVV | M29;M79;M22 | KCEESSAKS | M56;M7;M50;M52;M26 |
| 1970 | FNSAIGKIQDSLSSTAS | M40;M64; | VAGGIVAIVVTCLAY | M60;M6;M62;M22;M59; |
| | ALGKLQDV | M29;M79;M22 | YFMRFRRAFG | M60;M6;M62;M22;M59 |
| 1971 | QFNSAIGKIQDSLSSTA | M40;M64; | DVTKIKPHNSHEGKT | M1;M53;M74;M37;M42;M1; |
| | SALGKLQD | M29;M79;M22 | FYVLPNDDTL | M53;M74;M37;M42 |
| 1972 | NQFNSAIGKIQDSLSST | M40;M64; | ADVTKIKPHNSHEGK | M1;M53;M74;M37;M42;M1; |
| | ASALGKLQ | M29;M79;M22 | TFYVLPNDDT | M53;M74;M37;M42 |
| 1973 | PVPEVKILNNLGVDIA | M11;M54; | VRSIFSRTLETAQNSV | M54;M24;M34;M2;M9;M54; |
| | ANTVIWDYK | M57;M27;M9 | RVLQKAAIT | M24;M34;M2;M9 |
| 1974 | NPDILRVYANLGERVR | M21;M32; | LFQHANLDSCKRVLN | M58;M5;M2;M27;M15;M58; |
| | QALLKTVQF | M22;M49;M11 | VVCKTCGQQQ | M5;M2;M27;M15 |
| 1975 | KWYIRVGARKSAPLIE | M64;M40; | DCVSFCYMHHMELPT | M28;M18;M17;M43;M52; |
| | LCVDEAGSK | M57;M22;M23 | GVHAGTDLEG | M28;M18;M17;M43;M52 |
| 1976 | LQPRTFLLKYNENGTI | M58;M24; | YDCVSFCYMHHMEL | M28;M18;M17;M43;M52; |
| | TDAVDCALD | M49;M15;M11 | PTGVHAGTDLE | M28;M18;M17;M43;M52 |
| 1977 | VSDIDYVPLKSATCITR | M34;M18; | NVYLAVFDKNLYDK | M56;M55;M43;M42;M39; |
| | CNLGGAVC | M43;M42;M39 | LVSSFLEMKSE | M56;M55;M43;M42;M39 |
| 1978 | FAIGLALYYPSARIVYT | M21;M5; | YIICISTKHFYWFFSN | M24;M7;M50;M18;M23; |
| | ACSHAAVD | M29;M38;M17 | YLKRRVVFN | M24;M7;M50;M18;M23 |
| 1979 | AFELWAKRNIKPVPEV | M54;M66; | VEVEKGVLPQLEQPY | M13;M28;M66;M74;M15; |
| | KILNNLGVD | M74;M49;M9 | VFIKRSDART | M13;M28;M66;M74;M15 |
| 1980 | FELWAKRNIKPVPEVK | M54;M66; | VLLSVLQQLRVESSSK | M54;M34;M30;M82;M11; |
| | ILNNLGVDI | M74;M49;M9 | LWAQCVQLH | M54;M34;M30;M82;M11 |
| 1981 | TLPKGIMMNVAKYTQ | M73;M58; | VVLLSVLQQLRVESSS | M54;M34;M30;M82;M11; |
| | LCQYLNTLTL | M78;M34;M11 | KLWAQCVQL | M54;M34;M30;M82;M11 |
| 1982 | DSYFVVKRHTFSNYQ | M71;M58; | VGDIILKPANNSLKIT | M28;M58;M34;M53;M26; |
| | HEETIYNLLK | M34;M18;M20 | EEVGHTDLM | M28;M58;M34;M53;M26 |
| 1983 | VLNDILSRLDKVEAEV | M58;M53; | ARTVYDDGARRVWT | M5;M2;M52;M47;M20;M5; |
| | QIDRLITGR | M50;M17;M15 | LMNVLTLVYKV | M2;M52;M47;M20 |
| 1984 | SVLNDILSRLDKVEAE | M58;M53; | TIEVNSFSGYLKLTDN | M38;M8;M9;M47;M23;M38; |
| | VQIDRLITG | M50;M17;M15 | VYIKNADIV | M8;M9;M47;M23 |
| 1985 | ISSVLNDILSRLDKVEA | M58;M53; | EVNSFSGYLKLTDNV | M38;M8;M9;M47;M23;M38; |
| | EVQIDRLI | M50;M17;M15 | YIKNADIVEE | M8;M9;M47;M23 |
| 1986 | SSVLNDILSRLDKVEA | M58;M53; | IEVNSFSGYLKLTDNV | M38;M8;M9;M47;M23;M38; |
| | EVQIDRLIT | M50;M17;M15 | YIKNADIVE | M8;M9;M47;M23 |
| 1987 | AISSVLNDILSRLDKVE | M58;M53; | TLQQIELKFNPPALQD | M75;M73;M58;M48;M74; |
| | AEVQIDRL | M50;M17;M15 | AYYRARAGE | M75;M73;M58;M48;M74 |
| 1988 | IVVFDEISMATNYDLS | M25;M50; | LTLQQIELKFNPPALQ | M75;M73;M58;M48;M74; |
| | VVNARLRAK | M18;M33;M26 | DAYYRARAG | M75;M73;M58;M48;M74 |
| 1989 | VFDEISMATNYDLSVV | M25;M50; | GVLTAVVIPTKKAGG | M58;M16;M53;M67;M17; |
| | NARLRAKHY | M18;M33;M26 | TTEMLAKALR | M58;M16;M53;M67;M17 |
| 1990 | VVFDEISMATNYDLSV | M25;M50; | KDGTCGLVEVEKGVL | M13;M28;M66;M74;M9; |
| | VNARLRAKH | M18;M33;M26 | PQLEQPYVFI | M13;M28;M66;M74;M9 |
| 1991 | CDGGSLYVNKHAFHT | M56;M73; | GTCGLVEVEKGVLPQ | M13;M28;M66;M74;M9; |
| | PAFDKSAFVN | M58;M50;M11 | LEQPYVFIKR | M13;M28;M66;M74;M9 |
| 1992 | DGGSLYVNKHAFHTP | M56;M73; | DGTCGLVEVEKGVLP | M13;M28;M66;M74;M9; |
| | AFDKSAFVNL | M58;M50;M11 | QLEQPYVFIK | M13;M28;M66;M74;M9 |
| 1993 | LSNLNLPGCDGGSLYV | M56;M73; | ARQHLKDGTCGLVEV | M13;M28;M24;M74;M9; |
| | NKHAFHTPA | M58;M50;M11 | EKGVLPQLEQ | M13;M28;M24;M74;M9 |
| 1994 | SNLNLPGCDGGSLYV | M56;M73; | RQHLKDGTCGLVEVE | M13;M28;M24;M74;M9; |
| | NKHAFHTPAF | M58;M50;M11 | KGVLPQLEQP | M13;M28;M24;M74;M9 |
| 1995 | EGVFVSNGTHWFVTQ | M24;M31; | EARQHLKDGTCGLVE | M13;M28;M24;M74;M9; |
| | RNFYEPQIIT | M50;M18;M35 | VEKGVLPQLE | M13;M28;M24;M74;M9 |
| 1996 | YGDCLGDIAARDLICA | M58;M78; | EVVLKKLKKSLNVAK | M32;M28;M74;M33;M68; |
| | QKFNGLTVL | M65;M79;M50 | SEFDRDAAMQ | M32;M28;M74;M33;M68 |
| 1997 | QYGDCLGDIAARDLIC | M58;M78; | VVLKKLKKSLNVAKS | M32;M28;M74;M33;M68; |
| | AQKFNGLTV | M65;M79;M50 | EFDRDAAMQR | M32;M28;M74;M33;M68 |
| 1998 | KQYGDCLGDIAARDLI | M58;M78; | SEVVLKKLKKSLNVA | M32;M28;M74;M33;M68; |
| | CAQKFNGLT | M65;M79;M50 | KSEFDRDAAM | M32;M28;M74;M33;M68 |
| 1999 | ARKHTTCCSLSHRFYR | M5;M60; | VLKKLKKSLNVAKSE | M32;M28;M74;M33;M68; |
| | LANECAQVL | M79;M43;M42 | FDRDAAMQRK | M32;M28;M74;M33;M68 |
| 2000 | FHLYLQYIRKLHDELT | M32;M52; | KLKKSLNVAKSEFDR | M32;M28;M50;M33;M68; |
| | GHMLDMYSV | M38;M46;M9 | DAAMQRKLEK | M32;M28;M50;M33;M68 |
| 2001 | VFHLYLQYIRKLHDEL | M32;M52; | NIVTRCLNRVCTNYM | M64;M6;M37;M69;M20; |
| | TGHMLDMYS | M38;M46;M9 | PYFFTLLLQL | M64;M6;M37;M69;M20 |
| 2002 | FDGRVDGQVDLFRNA | M10;M33; | TTTNIVTRCLNRVCTN | M64;M6;M37;M69;M20; |
| | RNGVLITEGS | M41;M43;M42 | YMPYFFTLL | M64;M6;M37;M69;M20 |
| 2003 | VDGQVDLFRNARNGV | M10;M33; | IVTRCLNRVCTNYMP | M64;M6;M37;M69;M20; |
| | LITEGSVKGL | M41;M43;M42 | YFFTLLLQLC | M64;M6;M37;M69;M20 |
| 2004 | RVDGQVDLFRNARNG | M10;M33; | TNIVTRCLNRVCTNY | M64;M6;M37;M69;M20; |
| | VLITEGSVKG | M41;M43;M42 | MPYFFTLLLQ | M64;M6;M37;M69;M20 |
| 2005 | DGRVDGQVDLFRNAR | M10;M33; | TTNIVTRCLNRVCTN | M64;M6;M37;M69;M20; |
| | NGVLITEGSV | M41;M43;M42 | YMPYFFTLLL | M64;M6;M37;M69;M20 |
| 2006 | GRVDGQVDLFRNARN | M10;M33; | KGTHHWLLLTILTSLL | M52;M42;M43;M46;M39; |
| | GVLITEGSVK | M41;M43;M42 | VLVQSTQWS | M52;M42;M43;M46;M39 |
| 2007 | SINFVRIIMRLWLCWK | M75;M24; | GTHHWLLLTILTSLLV | M52;M42;M43;M46;M39; |
| | CRSKNPLLY | M76;M9;M23 | LVQSTQWSL | M52;M42;M43;M46;M39 |
| 2008 | QSINFVRIIMRLWLCW | M75;M24; | TFFPDLNGDVVAIDY | M7;M60;M37;M69;M82;M7; |
| | KCRSKNPLL | M76;M9;M23 | KHYTPSFKKG | M60;M37;M69;M82 |
| 2009 | FNKKDWYDFVENPDI | M21;M32; | PDLNGDVVAIDYKHY | M7;M60;M37;M69;M82;M7; |
| | LRVYANLGER | M43;M49;M42 | TPSFKKGAKL | M60;M37;M69;M82 |
| 2010 | THWFVTQRNFYEPQII | M64;M5; | FPDLNGDVVAIDYKH | M7;M60;M37;M69;M82;M7; |
| | TTDNTFVSG | M79;M9;M23 | YTPSFKKGAK | M60;M37;M69;M82 |
| 2011 | YDHVISTSHKLVLSVN | M64;M24; | DLNGDVVAIDYKHYT | M7;M60;M37;M69;M82;M7; |
| | PYVCNAPGC | M10;M9;M23 | PSFKKGAKLL | M60;M37;M69;M82 |
| 2012 | CYDHVISTSHKLVLSV | M64;M24; | FFPDLNGDVVAIDYK | M7;M60;M37;M69;M82;M7; |
| | NPYVCNAPG | M10;M9;M23 | HYTPSFKKGA | M60;M37;M69;M82 |
| 2013 | RRPFLCCKCCYDHVIS | M32;M24; | LNGDVVAIDYKHYTP | M7;M60;M37;M69;M82;M7; |
| | TSHKLVLSV | M10;M9;M23 | SFKKGAKLLH | M60;M37;M69;M82 |
| 2014 | IRRPFLCCKCCYDHVIS | M32;M24; | VAYFNMVYMPASWV | M52;M83;M15;M46;M39; |
| | TSHKLVLS | M10;M9;M23 | MRIMTWLDMVD | M52;M83;M15;M46;M39 |
| 2015 | YFLQSINFVRIIMRLWL | M75;M24; | AYFNMVYMPASWV | M52;M83;M15;M46;M39; |
| | CWKCRSKN | M10;M9;M23 | MRIMTWLDMVDT | M52;M83;M15;M46;M39 |
| 2016 | DDIVKTDGTLMIERFV | M4;M10; | VSSFLEMKSEKQVEQ | M31;M25;M34;M74;M39; |
| | SLAIDAYPL | M1;M5;M2;M3 | KIAEIPKEEV | M31;M25;M34;M74;M39 |
| 2017 | SANNCTFEYVSQPFLM | M4;M1;M3; | YNRYLALYNKYKYFS | M66;M31;M55;M67;M20; |
| | DLEGKQGNF | M57;M36;M20 | GAMDTTSYRE | M66;M31;M55;M67;M20 |
| 2018 | ANNCTFEYVSQPFLM | M4;M1;M3; | QYNRYLALYNKYKY | M66;M31;M55;M67;M20; |
| | DLEGKQGNFK | M57;M36;M20 | FSGAMDTTSYR | M66;M31;M55;M67;M20 |
| 2019 | SSANNCTFEYVSQPFL | M4;M1;M3; | GRWVLNNDYYRSLP | M34;M33;M43;M42;M39; |
| | MDLEGKQGN | M57;M36;M20 | GVFCGVDAVNL | M34;M33;M43;M42;M39 |
| 2020 | PRTLLTKGTLEPEYFN | M4;M13; | RWVLNNDYYRSLPG | M34;M33;M43;M42;M39; |
| | SVCRLMKTI | M16;M1;M3;M6 | VFCGVDAVNLL | M34;M33;M43;M42;M39 |
| 2021 | AQLPAPRTLLTKGTLE | M4;M58;M1; | KGEDIQLLKSAYENF | M32;M28;M60;M38;M68; |
| | PEYFNSVCR | M29;M3;M6 | NQHEVLLAPL | M32;M28;M60;M38;M68 |
| 2022 | QLPAPRTLLTKGTLEP | M4;M58;M1; | NKGEDIQLLKSAYEN | M32;M28;M60;M38;M68; |
| | EYFNSVCRL | M29;M3;M6 | FNQHEVLLAP | M32;M28;M60;M38;M68 |
| 2023 | WHHSIGFDYVYNPFMI | M4;M1;M3; | VQSKMSDVKCTSVVL | M40;M56;M54;M29;M22; |
| | DVQQWGFTG | M57;M43;M20 | LSVLQQLRVE | M40;M56;M54;M29;M22 |
| 2024 | YACWHHSIGFDYVYN | M4;M1;M3; | SKMSDVKCTSVVLLS | M40;M56;M54;M29;M22; |
| | PFMIDVQQWG | M57;M43;M20 | VLQQLRVESS | M40;M56;M54;M29;M22 |
| 2025 | CWHHSIGFDYVYNPF | M4;M1;M3; | QSKMSDVKCTSVVLL | M40;M56;M54;M29;M22; |
| | MIDVQQWGFT | M57;M43;M20 | SVLQQLRVES | M40;M56;M54;M29;M22 |
| 2026 | HHSIGFDYVYNPFMID | M4;M1;M3; | KGFCDLKGKYVQIPT | M40;M31;M30;M43;M42; |
| | VQQWGFTGN | M57;M43;M20 | TCANDPVGFT | M40;M31;M30;M43;M42 |
| 2027 | ACWHHSIGFDYVYNP | M4;M1;M3; | GFCDLKGKYVQIPTT | M40;M31;M30;M43;M42; |
| | FMIDVQQWGF | M57;M43;M20 | CANDPVGFTL | M40;M31;M30;M43;M42 |
| 2028 | VDSSQGSEYDYVIFTQ | M4;M64;M3; | FCDLKGKYVQIPTTC | M40;M31;M30;M43;M42; |
| | TTETAHSCN | M51;M22;M23 | ANDPVGFTLK | M40;M31;M30;M43;M42 |
| 2029 | SSQGSEYDYVIFTQTT | M4;M64;M3; | PKGFCDLKGKYVQIP | M40;M31;M30;M43;M42; |
| | ETAHSCNVN | M51;M22;M23 | TTCANDPVGF | M40;M31;M30;M43;M42 |
| 2030 | DSSQGSEYDYVIFTQT | M4;M64;M3; | CDLKGKYVQIPTTCA | M40;M31;M30;M43;M42; |
| | TETAHSCNV | M51;M22;M23 | NDPVGFTLKN | M40;M31;M30;M43;M42 |
| 2031 | FHPLADNKFALTCFST | M4;M21;M14; | SWLMWLIINLVQMAP | M9;M73;M55;M50;M11; |
| | QFAFACPDG | M3;M6;M22 | ISAMVRMYIF | M9;M73;M55;M50;M11 |
| 2032 | KWADNNCYLATALLT | M4;M51;M3; | LTAVVIPTKKAGGTT | M58;M16;M78;M53;M17; |
| | LQQIELKFNP | M4;M51;M3 | EMLAKALRKV | M58;M16;M78;M53;M17 |
| 2033 | WADNNCYLATALLTL | M4;M51;M3; | VLTAVVIPTKKAGGT | M58;M16;M78;M53;M17; |
| | QQIELKFNPP | M4;M51;M3 | TEMLAKALRK | M58;M16;M78;M53;M17 |
| 2034 | DNNCYLATALLTLQQI | M4;M51;M3; | HVVGPNVNKGEDIQL | M77;M32;M28;M60;M68; |
| | ELKFNPPAL | M4;M51;M3 | LKSAYENFNQ | M77;M32;M28;M60;M68 |
| 2035 | SIKWADNNCYLATAL | M4;M51;M3; | TQYNRYLALYNKYK | M31;M55;M67;M27;M20; |
| | LTLQQIELKF | M4;M51;M3 | YFSGAMDTTSY | M31;M55;M67;M27;M20 |
| 2036 | ADNNCYLATALLTLQ | M4;M51;M3; | LTQYNRYLALYNKY | M55;M67;M27;M42;M20; |
| | QIELKFNPPA | M4;M51;M3 | KYFSGAMDTTS | M55;M67;M27;M42;M20 |
| 2037 | IKWADNNCYLATALL | M4;M51;M3; | YHNESGLKTILRKGG | M13;M58;M16;M53;M17; |
| | TLQQIELKFN | M4;M51;M3 | RTIAFGGCVF | M13;M58;M16;M53;M17 |
| 2038 | ITNLCPFGEVFNATRF | M4;M58;M3; | LAEYHNESGLKTILRK | M13;M58;M16;M53;M17; |
| | ASVYAWNRK | M2;M17;M20 | GGRTIAFGG | M13;M58;M16;M53;M17 |
| 2039 | PLADNKFALTCFSTQF | M4;M21;M3; | AEYHNESGLKTILRK | M13;M58;M16;M53;M17; |
| | AFACPDGVK | M6;M33;M22 | GGRTIAFGGC | M13;M58;M16;M53;M17 |
| 2040 | YRNRDVDTDFVNEFY | M4;M71;M6; | EYHNESGLKTILRKG | M13;M58;M16;M53;M17; |
| | AYLRKHFSMM | M3;M26;M59 | GRTIAFGGCV | M13;M58;M16;M53;M17 |
| 2041 | RNRDVDTDFVNEFYA | M4;M71;M6; | MWLIINLVQMAPISA | M9;M73;M55;M11;M23; |
| | YLRKHFSMMI | M3;M26;M59 | MVRMYIFFAS | M9;M73;M55;M11;M23 |
| 2042 | ISNCVADYSVLYNSAS | M73;M7;M3; | QTMLFTMLRKLDND | M16;M43;M15;M52;M9; |
| | FSTFKCYGV | M15;M11;M44 | ALNNIINNARD | M16;M43;M15;M52;M9 |
| 2043 | FFSNYLKRRVVFNGVS | M4;M3;M6; | MQTMLFTMLRKLDN | M16;M43;M15;M52;M9; |
| | FSTFEEAAL | M4;M3;M6 | DALNNIINNAR | M16;M43;M15;M52;M9 |
| 2044 | FSNYLKRRVVFNGVSF | M4;M3;M6; | PYIVGDVVQEGVLTA | M29;M38;M61;M67;M27; |
| | STFEEAALC | M4;M3;M6 | VVIPTKKAGG | M29;M38;M61;M67;M27 |
| 2045 | NCVADYSVLYNSASFS | M73;M3;M41; | YIVGDVVQEGVLTAV | M29;M38;M61;M67;M27; |
| | TFKCYGVSP | M15;M11;M44 | VIPTKKAGGT | M29;M38;M61;M67;M27 |
| 2046 | SNCVADYSVLYNSASF | M73;M3;M41; | TGTGQAITVTPEANM | M24;M78;M34;M29;M49; |
| | STFKCYGVS | M15;M11;M44 | DQESFGGASC | M24;M78;M34;M29;M49 |
| 2047 | CVADYSVLYNSASFST | M73;M3;M41; | DSCNNYMLTYNKVE | M53;M74;M15;M52;M11; |
| | FKCYGVSPT | M15;M11;M44 | NMTPRDLGACI | M53;M74;M15;M52;M11 |
| 2048 | VLKSEDAQGMDNLAC | M1;M3;M37; | LFDESGEFKLASHMY | M16;M20;M15;M52;M23; |
| | EDLKPVSEEV | M1;M3;M37 | CSFYPPDEDE | M16;M20;M15;M52;M23 |
| 2049 | LTGYKKPASRELKVTF | M57;M63;M3; | FDESGEFKLASHMYC | M16;M20;M15;M52;M23; |
| | FPDLNGDVV | M57;M63;M3 | SFYPPDEDEE | M16;M20;M15;M52;M23 |
| 2050 | TSKFYGGWHNMLKTV | M32;M6;M3; | YLFDESGEFKLASHM | M16;M20;M15;M52;M23; |
| | YSDVENPHLM | M18;M43;M59 | YCSFYPPDED | M16;M20;M15;M52;M23 |
| 2051 | YVLPNDDTLRVEAFEY | M41;M3;M6; | YYLFDESGEFKLASH | M16;M20;M15;M52;M23; |
| | YHTTDPSFL | M41;M3;M6 | MYCSFYPPDE | M16;M20;M15;M52;M23 |
| 2052 | VLPNDDTLRVEAFEYY | M41;M3;M6; | FLGRYMSALNHTKK | M24;M5;M25;M74;M33; |
| | HTTDPSFLG | M41;M3;M6 | WKYPQVNGLTS | M24;M5;M25;M74;M33 |
| 2053 | GHTDLMAAYVDNSSL | M51;M63;M44; | LGRYMSALNHTKKW | M24;M5;M25;M74;M33; |
| | TIKKPNELSR | M51;M63;M44 | KYPQVNGLTSI | M24;M5;M25;M74;M33 |
| 2054 | DIAARDLICAQKFNGL | M58;M78;M65; | VVDYGARFYFYTSKT | M40;M29;M41;M15;M47; |
| | TVLPPLLTD | M10;M3;M79 | TVASLINTLN | M40;M29;M41;M15;M47 |
| 2055 | GDIAARDLICAQKFNG | M58;M78;M65; | VDYGARFYFYTSKTT | M40;M29;M41;M15;M47; |
| | LTVLPPLLT | M10;M3;M79 | VASLINTLND | M40;M29;M41;M15;M47 |
| 2056 | LGDIAARDLICAQKFN | M58;M78;M65; | DYGARFYFYTSKTTV | M40;M29;M41;M15;M47; |
| | GLTVLPPLL | M10;M3;M79 | ASLINTLNDL | M40;M29;M41;M15;M47 |
| 2057 | DKRAKVTSAMQTMLF | M52;M3;M16; | EGVVDYGARFYFYTS | M40;M29;M41;M15;M47; |
| | TMLRKLDNDA | M52;M3;M16 | KTTVASLINT | M40;M29;M41;M15;M47 |
| 2058 | ATHGLAAVNSVPWDT | M55;M54;M3; | GVVDYGARFYFYTSK | M40;M29;M41;M15;M47; |
| | IANYAKPFLN | M55;M54;M3 | TTVASLINTL | M40;M29;M41;M15;M47 |
| 2059 | NDVSFLAHIQWMVMF | M30;M3;M58; | QEAYEQAVANGDSE | M13;M55;M53;M33;M59; |
| | TPLVPFWITI | M30;M3;M58 | VVLKKLKKSLN | M13;M55;M53;M33;M59 |
| 2060 | MFLARGIVFMCVEYCP | M22;M3;M20; | KMLCTHTGTGQAITV | M24;M78;M34;M29;M9; |
| | IFFITGNTL | M22;M3;M20 | TPEANMDQES | M24;M78;M34;M29;M9 |
| 2061 | PDVDLGDISGINASVV | M28;M14;M70; | CDTFCAGSTFISDEVA | M31;M70;M57;M35;M20; |
| | NIQKEIDRL | M80;M45;M47 | RDLSLQFKR | M31;M70;M57;M35;M20 |
| 2062 | VDLGDISGINASVVNI | M28;M14;M70; | VNCDTFCAGSTFISDE | M31;M70;M57;M35;M20; |
| | QKEIDRLNE | M80;M45;M47 | VARDLSLQF | M31;M70;M57;M35;M20 |
| 2063 | DVDLGDISGINASVVN | M28;M14;M70; | NCDTFCAGSTFISDEV | M31;M70;M57;M35;M20; |
| | IQKEIDRLN | M80;M45;M47 | ARDLSLQFK | M31;M70;M57;M35;M20 |
| 2064 | SPDVDLGDISGINASV | M28;M14;M70; | MSALNHTKKWKYPQ | M54;M24;M5;M60;M74; |
| | VNIQKEIDR | M80;M45;M47 | VNGLTSIKWAD | M54;M24;M5;M60;M74 |
| 2065 | EDKRAKVTSAMQTML | M52;M3;M62; | YMSALNHTKKWKYP | M54;M24;M5;M60;M74; |
| | FTMLRKLDND | M52;M3;M62 | QVNGLTSIKWA | M54;M24;M5;M60;M74 |
| 2066 | EEVTTTLEETKFLTEN | M50;M3;M20; | SALNHTKKWKYPQV | M54;M24;M5;M60;M74; |
| | LLLYIDING | M50;M3;M20 | NGLTSIKWADN | M54;M24;M5;M60;M74 |
| 2067 | QLFFSYFAVHFISNSW | M9;M3;M23; | DVVAIDYKHYTPSFK | M16;M37;M69;M27;M82; |
| | LMWLIINLV | M9;M3;M23 | KGAKLLHKPI | M16;M37;M69;M27;M82 |
| 2068 | LFFSYFAVHFISNSWL | M9;M3;M23; | VVAIDYKHYTPSFKK | M16;M37;M69;M27;M82; |
| | MWLIINLVQ | M9;M3;M23 | GAKLLHKPIV | M16;M37;M69;M27;M82 |
| 2069 | FFSYFAVHFISNSWLM | M9;M3;M23; | NGDVVAIDYKHYTPS | M16;M60;M37;M69;M82; |
| | WLIINLVQM | M9;M3;M23 | FKKGAKLLHK | M16;M60;M37;M69;M82 |
| 2070 | MQLFFSYFAVHFISNS | M9;M3;M23; | ASFDNFKFVCDNIKFA | M40;M64;M21;M22;M23; |
| | WLMWLIINL | M9;M3;M23 | DDLNQLTGY | M40;M64;M21;M22;M23 |
| 2071 | FSYFAVHFISNSWLM | M9;M3;M23; | FDNFKFVCDNIKFAD | M40;M64;M21;M22;M23; |
| | WLIINLVQMA | M9;M3;M23 | DLNQLTGYKK | M40;M64;M21;M22;M23 |
| 2072 | CIPYNSVTSSIVITSGD | M12;M14;M29; | DNFKFVCDNIKFADD | M40;M64;M21;M22;M23; |
| | GTTSPISE | M45;M62;M43 | LNQLTGYKKP | M40;M64;M21;M22;M23 |
| 2073 | PDKVFRSSVLHSTQDL | M4;M1;M29; | SFDNFKFVCDNIKFAD | M40;M64;M21;M22;M23; |
| | FLPFFSNVT | M6;M67;M72 | DLNQLTGYK | M40;M64;M21;M22;M23 |
| 2074 | NCTFEYVSQPFLMDLE | M4;M1;M57; | PEEHFIETISLAGSYKD | M64;M40;M32;M29;M68; |
| | GKQGNFKNL | M36;M46;M20 | WSYSGQST | M64;M40;M32;M29;M68 |
| 2075 | LTSHTVMPLSAPTLVP | M28;M54;M51; | LKPVLDWLEEKFKEG | M61;M57;M27;M9;M20; |
| | QEHYVRITG | M69;M46;M39 | VEFLRDGWEI | M61;M57;M27;M9;M20 |
| 2076 | TSHTVMPLSAPTLVPQ | M28;M54;M51; | KLKPVLDWLEEKFKE | M61;M57;M27;M9;M20; |
| | EHYVRITGL | M69;M46;M39 | GVEFLRDGWE | M61;M57;M27;M9;M20 |
| 2077 | LWLLWPVTLACFVLA | M58;M14;M70; | TAQNSVRVLQKAAITI | M56;M54;M24;M5;M9; |
| | AVYRINWITG | M33;M63;M44 | LDGISQYSL | M56;M54;M24;M5;M9 |
| 2078 | WLLWPVTLACFVLAA | M58;M14;M70; | ETAQNSVRVLQKAAI | M56;M54;M24;M5;M9; |
| | VYRINWITGG | M33;M63;M44 | TILDGISQYS | M56;M54;M24;M5;M9 |
| 2079 | WPVTLACFVLAAVYRI | M58;M14;M70; | GQPITNCVKMLCTHT | M64;M21;M58;M22;M23; |
| | NWITGGIAI | M33;M63;M44 | GTGQAITVTP | M64;M21;M58;M22;M23 |
| 2080 | LLWPVTLACFVLAAV | M58;M14;M70; | GKASCTLSEQLDFIDT | M64;M74;M83;M27;M22; |
| | YRINWITGGI | M33;M63;M44 | KRGVYCCRE | M64;M74;M83;M27;M22 |
| 2081 | LWPVTLACFVLAAVY | M58;M14;M70; | AKNTVKSVGKFCLEA | M24;M25;M43;M42;M9; |
| | RINWITGGIA | M33;M63;M44 | SFNYLKSPNF | M24;M25;M43;M42;M9 |
| 2082 | VFTTVDNINLHTQVVD | M51;M67;M6; | IAKNTVKSVGKFCLE | M24;M25;M43;M42;M9; |
| | MSMTYGQQF | M51;M67;M6 | ASFNYLKSPN | M24;M25;M43;M42;M9 |
| 2083 | FTTVDNINLHTQVVD | M51;M67;M6; | SAVKRTIKGTHHWLL | M24;M42;M43;M52;M9; |
| | MSMTYGQQFG | M51;M67;M6 | LTILTSLLVL | M24;M42;M43;M52;M9 |
| 2084 | IKVFTTVDNINLHTQV | M51;M67;M6; | DYDCVSFCYMHHME | M18;M43;M17;M52;M9; |
| | VDMSMTYGQ | M51;M67;M6 | LPTGVHAGTDL | M18;M43;M17;M52;M9 |
| 2085 | TTVDNINLHTQVVDM | M51;M67;M6; | SWVMRIMTWLDMVD | M64;M75;M70;M83;M22; |
| | SMTYGQQFGP | M51;M67;M6 | TSLSGFKLKDC | M64;M75;M70;M83;M22 |
| 2086 | TVDNINLHTQVVDMS | M51;M67;M6; | VMRIMTWLDMVDTS | M64;M75;M70;M83;M22; |
| | MTYGQQFGPT | M51;M67;M6 | LSGFKLKDCVM | M64;M75;M70;M83;M22 |
| 2087 | KVFTTVDNINLHTQVV | M51;M67;M6; | WVMRIMTWLDMVDT | M64;M75;M70;M83;M22; |
| | DMSMTYGQQ | M51;M67;M6 | SLSGFKLKDCV | M64;M75;M70;M83;M22 |
| 2088 | APFLYLYALVYFLQSI | M4;M19;M54; | IVEEAKKVKPTVVVN | M56;M73;M18;M11;M23; |
| | NFVRIIMRL | M12;M60;M25 | AANVYLKHGG | M56;M73;M18;M11;M23 |
| 2089 | LSSNFGAISSVLNDILS | M34;M6;M45; | DIVEEAKKVKPTVVV | M56;M73;M18;M11;M23; |
| | RLDKVEAE | M48;M50;M17 | NAANVYLKHG | M56;M73;M18;M11;M23 |
| 2090 | VNALPETTADIVVFDEI | M28;M55;M7; | GLMLLEIKDTEKYCA | M78;M65;M34;M82;M9; |
| | SMATNYDL | M38;M48;M49 | LAPNMMVTNN | M78;M65;M34;M82;M9 |
| 2091 | SETKCTLKSFTVEKGI | M75;M21;M14; | VKSREETGLLMPLKA | M54;M31;M60;M38;M27; |
| | YQTSNFRVQ | M25;M63;M8 | PKEIIFLEGE | M54;M31;M60;M38;M27 |
| 2092 | QNCVLKLKVDTANPK | M51;M7;M77; | SREETGLLMPLKAPK | M54;M31;M60;M38;M27; |
| | TPKYKFVRIQ | M51;M7;M77 | EIIFLEGETL | M54;M31;M60;M38;M27 |
| 2093 | GHSMQNCVLKLKVDT | M51;M7;M77; | EETGLLMPLKAPKEII | M54;M31;M60;M38;M27; |
| | ANPKTPKYKF | M51;M7;M77 | FLEGETLPT | M54;M31;M60;M38;M27 |
| 2094 | MQNCVLKLKVDTANP | M51;M7;M77; | REETGLLMPLKAPKEI | M54;M31;M60;M38;M27; |
| | KTPKYKFVRI | M51;M7;M77 | IFLEGETLP | M54;M31;M60;M38;M27 |
| 2095 | NCVLKLKVDTANPKT | M51;M7;M77; | KSREETGLLMPLKAP | M54;M31;M60;M38;M27; |
| | PKYKFVRIQP | M51;M7;M77 | KEIIFLEGET | M54;M31;M60;M38;M27 |
| 2096 | SMQNCVLKLKVDTAN | M51;M7;M77; | PTEVLTEEVVLKTGD | M58;M60;M38;M17;M62; |
| | PKTPKYKFVR | M51;M7;M77 | LQPLEQPTSE | M58;M60;M38;M17;M62 |
| 2097 | HSMQNCVLKLKVDTA | M51;M7;M77; | SEGLNDNLLEILQKEK | M24;M10;M61;M27;M9; |
| | NPKTPKYKFV | M51;M7;M77 | VNINIVGDF | M24;M10;M61;M27;M9 |
| 2098 | IGHSMQNCVLKLKVD | M51;M7;M77; | EGSEGLNDNLLEILQK | M24;M10;M61;M27;M9; |
| | TANPKTPKYK | M51;M7;M77 | EKVNINIVG | M24;M10;M61;M27;M9 |
| 2099 | TDQSSYIVDSVTVKNG | M36;M12;M48; | VVGEGSEGLNDNLLE | M24;M10;M61;M27;M9; |
| | SIHLYFDKA | M36;M12;M48 | ILQKEKVNIN | M24;M10;M61;M27;M9 |
| 2100 | DQSSYIVDSVTVKNGS | M36;M12;M48; | EGLNDNLLEILQKEK | M24;M10;M61;M27;M9; |
| | IHLYFDKAG | M36;M12;M48 | VNINIVGDFK | M24;M10;M61;M27;M9 |

TABLE 2Aii
Peptides and Alleles

| | Set 1 | Set 1 |
|---|---|---|
| | Peptide | Allele |
| 1 | GEGSEGLNDNLLEIL | M24;M10;M61;M27;M9;M24;M10;M61;M27;M9 |
| | QKEKVNINIV | |
| 2 | VGEGSEGLNDNLLE | M24;M10;M61;M27;M9;M24;M10;M61;M27;M9 |
| | ILQKEKVNINI | |
| 3 | GSEGLNDNLLEILQ | M24;M10;M61;M27;M9;M24;M10;M61;M27;M9 |
| | KEKVNINIVGD | |
| 4 | TGVVGEGSEGLNDN | M24;M10;M61;M27;M9;M24;M10;M61;M27;M9 |
| | LLEILQKEKVN | |
| 5 | GVVGEGSEGLNDNL | M24;M10;M61;M27;M9;M24;M10;M61;M27;M9 |
| | LEILQKEKVNI | |
| 6 | AVKRTIKGTHHWLL | M34;M42;M43;M52;M9;M34;M42;M43;M52;M9 |
| | LTILTSLLVLV | |
| 7 | KRTIKGTHHWLLLTI | M34;M42;M43;M52;M9;M34;M42;M43;M52;M9 |
| | LTSLLVLVQS | |
| 8 | VKRTIKGTHHWLLL | M34;M42;M43;M52;M9;M34;M42;M43;M52;M9 |
| | TILTSLLVLVQ | |
| 9 | VYMPASWVMRIMT | M32;M70;M83;M15;M52;M32;M70;M83;M15;M52 |
| | WLDMVDTSLSGF | |
| 10 | MVYMPASWVMRIM | M32;M70;M83;M15;M52;M32;M70;M83;M15;M52 |
| | TWLDMVDTSLSG | |
| 11 | NMVYMPASWVMRI | M32;M70;M83;M15;M52;M32;M70;M83;M15;M52 |
| | MTWLDMVDTSLS | |
| 12 | LDMCASLKELLQNG | M56;M24;M60;M50;M18;M56;M24;M60;M50;M18 |
| | MNGRTILGSAL | |
| 13 | GGRFVLALLSDLQD | M77;M32;M54;M70;M60;M77;M32;M54;M70;M60 |
| | LKWARFPKSDG | |
| 14 | PISAMVRMYIFFASF | M24;M20;M35;M9;M23;M24;M20;M35;M9;M23 |
| | YYVWKSYVHV | |
| 15 | ISAMVRMYIFFASFY | M24;M20;M35;M9;M23;M24;M20;M35;M9;M23 |
| | YVWKSYVHVV | |
| 16 | QTGIAVLDMCASLK | M32;M24;M10;M60;M59;M32;M24;M10;M60;M59 |
| | ELLQNGMNGRT | |
| 17 | TGIAVLDMCASLKE | M32;M24;M10;M60;M59;M32;M24;M10;M60;M59 |
| | LLQNGMNGRTI | |
| 18 | SAQTGIAVLDMCAS | M32;M24;M10;M60;M59;M32;M24;M10;M60;M59 |
| | LKELLQNGMNG | |
| 19 | AQTGIAVLDMCASL | M32;M24;M10;M60;M59;M32;M24;M10;M60;M59 |
| | KELLQNGMNGR | |
| 20 | LSAQTGIAVLDMCA | M32;M24;M10;M60;M59;M32;M24;M10;M60;M59 |
| | SLKELLQNGMN | |
| 21 | KGLDYKAFKQIVES | M37;M42;M52;M47;M23;M37;M42;M52;M47;M23 |
| | CGNFKVTKGKA | |
| 22 | VKGLDYKAFKQIVE | M37;M42;M52;M47;M23;M37;M42;M52;M47;M23 |
| | SCGNFKVTKGK | |
| 23 | LKLRSDVLLPLTQY | M53;M61;M69;M27;M20;M53;M61;M69;M27;M20 |
| | NRYLALYNKYK | |
| 24 | LRSDVLLPLTQYNR | M53;M61;M69;M27;M20;M53;M61;M69;M27;M20 |
| | YLALYNKYKYF | |
| 25 | KLRSDVLLPLTQYN | M53;M61;M69;M27;M20;M53;M61;M69;M27;M20 |
| | RYLALYNKYKY | |
| 26 | FVDDIVKTDGTLMI | M4;M9;M10;M1;M5;M7;M2;M3;M6;M8;M11 |
| | ERFVSLAIDAY | |
| 27 | LTKGTLEPEYFNSV | M4;M13;M12;M14;M16;M19;M1;M3;M18;M17;M15 |
| | CRLMKTIGPDM | |
| 28 | KGTLEPEYFNSVCR | M4;M13;M12;M14;M16;M19;M3;M6;M18;M17;M15 |
| | LMKTIGPDMFL | |
| 29 | QIYKTPPIKDFGGFN | M4;M32;M28;M14;M31;M1;M29;M3;M33;M27;M35 |
| | FSQILPDPSK | |
| 30 | IYKTPPIKDFGGFNF | M4;M32;M28;M14;M31;M1;M29;M3;M33;M27;M35 |
| | SQILPDPSKP | |
| 31 | GTLEPEYFNSVCRL | M13;M12;M14;M16;M19;M5;M3;M6;M18;M17;M15 |
| | MKTIGPDMFLG | |
| 32 | FTVEKGIYQTSNFRV | M31;M1;M34;M30;M3;M37;M50;M41;M8;M39;M23 |
| | QPTESIVRFP | |
| 33 | KSFTVEKGIYQTSNF | M31;M1;M34;M30;M3;M37;M50;M41;M8;M39;M23 |
| | RVQPTESIVR | |
| 34 | SFTVEKGIYQTSNFR | M31;M1;M34;M30;M3;M37;M50;M41;M8;M39;M23 |
| | VQPTESIVRF | |
| 35 | SFKELLVYAADPAM | M64;M12;M30;M48;M57;M33;M36;M8;M39;M44;M20 |
| | HAASGNLLLDK | |
| 36 | MAGNGGDAALALL | M13;M56;M58;M16;M55;M10;M53;M51;M36;M17;M11 |
| | LLDRLNQLESKM | |
| 37 | NGGDAALALLLLDR | M13;M56;M58;M16;M55;M10;M53;M51;M36;M17;M11 |
| | LNQLESKMSGK | |
| 38 | AGNGGDAALALLLL | M13;M56;M58;M16;M55;M10;M53;M51;M36;M17;M11 |
| | DRLNQLESKMS | |
| 39 | GNGGDAALALLLLD | M13;M56;M58;M16;M55;M10;M53;M51;M36;M17;M11 |
| | RLNQLESKMSG | |
| 40 | EKGIYQTSNFRVQPT | M31;M34;M30;M48;M37;M50;M51;M41;M8;M39;M23 |
| | ESIVRFPNIT | |
| 41 | RCPAEIVDTVSALV | M12;M28;M16;M7;M29;M6;M38;M8;M17;M59;M23 |
| | YDNKLKAHKDK | |
| 42 | TNAGDYILANTCTE | M56;M73;M19;M31;M6;M44;M18;M63;M8;M52;M47 |
| | RLKLFAAETLK | |
| 43 | NAGDYILANTCTER | M56;M73;M19;M31;M6;M44;M18;M63;M8;M52;M47 |
| | LKLFAAETLKA | |
| 44 | SSRLSFKELLVYAA | M64;M30;M48;M57;M36;M41;M49;M26;M22;M44;M20 |
| | DPAMHAASGNL | |
| 45 | PLNRNYVFTGYRVT | M40;M14;M31;M24;M41;M8;M43;M42;M39;M9;M44 |
| | KNSKVQIGEYT | |
| 46 | VFTGYRVTKNSKVQ | M40;M14;M31;M24;M41;M8;M43;M42;M39;M9;M44 |
| | IGEYTFEKGDY | |
| 47 | RNYVFTGYRVTKNS | M40;M14;M31;M24;M41;M8;M43;M42;M39;M9;M44 |
| | KVQIGEYTFEK | |
| 48 | NRNYVFTGYRVTK | M40;M14;M31;M24;M41;M8;M43;M42;M39;M9;M44 |
| | NSKVQIGEYTFE | |
| 49 | YVFTGYRVTKNSKV | M40;M14;M31;M24;M41;M8;M43;M42;M39;M9;M44 |
| | QIGEYTFEKGD | |
| 50 | LNRNYVFTGYRVTK | M40;M14;M31;M24;M41;M8;M43;M42;M39;M9;M44 |
| | NSKVQIGEYTF | |
| 51 | FTGYRVTKNSKVQI | M40;M14;M31;M24;M41;M8;M43;M42;M39;M9;M44 |
| | GEYTFEKGDYG | |
| 52 | NYVFTGYRVTKNSK | M40;M14;M31;M24;M41;M8;M43;M42;M39;M9;M44 |
| | VQIGEYTFEKG | |
| 53 | WVYKQFDTYNLWN | M71;M58;M14;M16;M19;M25;M34;M53;M17;M15;M39 |
| | TFTRLQSLENVA | |
| 54 | VYKQFDTYNLWNT | M71;M58;M14;M16;M19;M25;M34;M53;M17;M15;M39 |
| | FTRLQSLENVAF | |
| 55 | EIPRRNVATLQAEN | M28;M54;M29;M6;M45;M38;M27;M46;M39;M59;M20 |
| | VTGLFKDCSKV | |
| 56 | LVKPSFYVYSRVKN | M40;M71;M12;M54;M16;M60;M38;M33;M8;M46;M39 |
| | LNSSRVPDLLV | |
| 57 | SLVKPSFYVYSRVK | M40;M71;M12;M54;M16;M60;M38;M33;M8;M46;M39 |
| | NLNSSRVPDLL | |
| 58 | DYNYKLPDDFTGCV | M13;M32;M12;M14;M29;M38;M48;M67;M68;M49;M26 |
| | IAWNSNNLDSK | |
| 59 | IPKDMTYRRLISMM | M40;M58;M16;M25;M30;M44;M46;M43;M42;M39;M47 |
| | GFKMNYQVNGY | |
| 60 | IKPVPEVKILNNLGV | M11;M9;M54;M60;M48;M57;M27;M43;M42;M22;M44 |
| | DIAANTVIWD | |
| 61 | NIKPVPEVKILNNLG | M11;M9;M54;M60;M48;M57;M27;M43;M42;M22;M44 |
| | VDIAANTVIW | |
| 62 | KRNIKPVPEVKILNN | M11;M9;M54;M60;M48;M57;M27;M43;M42;M22;M44 |
| | LGVDIAANTV | |
| 63 | RNIKPVPEVKILNNL | M11;M9;M54;M60;M48;M57;M27;M43;M42;M22;M44 |
| | GVDIAANTVI | |
| 64 | NFKDQVILLNKHID | M56;M73;M14;M53;M44;M41;M17;M35;M52;M11;M47 |
| | AYKTFPPTEPK | |
| 65 | FKDQVILLNKHIDA | M56;M73;M14;M53;M44;M41;M17;M35;M52;M11;M47 |
| | YKTFPPTEPKK | |
| 66 | PNFKDQVILLNKHID | M56;M73;M14;M53;M44;M41;M17;M35;M52;M11;M47 |
| | AYKTFPPTEP | |
| 67 | GLALYYPSARIVYT | M64;M21;M28;M14;M5;M29;M38;M17;M49;M46;M22 |
| | ACSHAAVDALC | |
| 68 | KVSIWNLDYIINLIIK | M56;M73;M28;M58;M16;M50;M18;M17;M15;M11;M44 |
| | NLSKSLTEN | |
| 69 | WNLDYIINLIIKNLS | M56;M73;M28;M58;M16;M50;M18;M17;M15;M11;M44 |
| | KSLTENKYSQ | |
| 70 | IWNLDYIINLIIKNLS | M56;M73;M28;M58;M16;M50;M18;M17;M15;M11;M44 |
| | KSLTENKYS | |
| 71 | VSIWNLDYIINLIIKN | M56;M73;M28;M58;M16;M50;M18;M17;M15;M11;M44 |
| | LSKSLTENK | |
| 72 | SIWNLDYIINLIIKNL | M56;M73;M28;M58;M16;M50;M18;M17;M15;M11;M44 |
| | SKSLTENKY | |
| 73 | NMFITREEAIRHVRA | M64;M32;M14;M34;M48;M57;M46;M69;M42;M39;M20 |
| | WIGFDVEGCH | |
| 74 | PNMFITREEAIRHVR | M64;M32;M14;M34;M48;M57;M46;M69;M42;M39;M20 |
| | AWIGFDVEGC | |
| 75 | FITREEAIRHVRAWI | M64;M32;M14;M34;M48;M57;M46;M69;M42;M39;M20 |
| | GFDVEGCHAT | |
| 76 | ITREEAIRHVRAWIG | M64;M32;M14;M34;M48;M57;M46;M69;M42;M39;M20 |
| | FDVEGCHATR | |
| 77 | MFITREEAIRHVRA | M64;M32;M14;M34;M48;M57;M46;M69;M42;M39;M20 |
| | WIGFDVEGCHA | |
| 78 | AKRNIKPVPEVKILN | M9;M54;M66;M60;M48;M57;M27;M43;M42;M22;M44 |
| | NLGVDIAANT | |
| 79 | ADSNGTITVEELKK | M4;M56;M58;M16;M55;M53;M6;M59;M17;M52;M47 |
| | LLEQWNLVIGF | |
| 80 | PDDFTGCVIAWNSN | M13;M32;M56;M12;M55;M48;M50;M67;M68;M49;M26 |
| | NLDSKVGGNYN | |
| 81 | ITRFQTLLALHRSYL | M23;M21;M24;M70;M55;M5;M10;M2;M48;M9;M20 |
| | TPGDSSSGWT | |
| 82 | NITRFQTLLALHRSY | M23;M21;M24;M70;M55;M5;M10;M2;M48;M9;M20 |
| | LTPGDSSSGW | |
| 83 | KLNVGDYFVLTSHT | M16;M55;M25;M34;M29;M18;M41;M43;M15;M42;M39 |
| | VMPLSAPTLVP | |
| 84 | TTYKLNVGDYFVLT | M16;M55;M25;M34;M29;M18;M41;M43;M15;M42;M39 |
| | SHTVMPLSAPT | |
| 85 | YKLNVGDYFVLTSH | M16;M55;M25;M34;M29;M18;M41;M43;M15;M42;M39 |
| | TVMPLSAPTLV | |
| 86 | TYKLNVGDYFVLTS | M16;M55;M25;M34;M29;M18;M41;M43;M15;M42;M39 |
| | HTVMPLSAPTL | |
| 87 | SQGLVASIKNFKSVL | M21; M54;M55;M60;M48;M41;M68;M49;M26;M22;M23 |
| | YYQNNVFMSE | |
| 88 | ASQGLVASIKNFKS | M21;M54;M55;M60;M48;M41;M68;M49;M26;M22;M23 |
| | VLYYQNNVFMS | |
| 89 | STYASQGLVASIKNF | M64;M21;M54;M55;M60;M48;M68;M49;M26;M22;M23 |
| | KSVLYYQNNV | |
| 90 | YASQGLVASIKNFK | M64;M21;M54;M55;M60;M48;M68;M49;M26;M22;M23 |
| | SVLYYQNNVFM | |
| 91 | TYASQGLVASIKNF | M64;M21;M54;M55;M60;M48;M68;M49;M26;M22;M23 |
| | KSVLYYQNNVF | |
| 92 | WQPGVAMPNLYKM | M12;M24;M55;M10;M53;M17;M35;M52;M9;M47;M23 |
| | QRMLLEKCDLQN | |
| 93 | AWQPGVAMPNLYK | M12;M24;M55;M10;M53;M17;M35;M52;M9;M47;M23 |
| | MQRMLLEKCDLQ | |
| 94 | QPGVAMPNLYKMQ | M12;M24;M55;M10;M53;M17;M35;M52;M9;M47;M23 |
| | RMLLEKCDLQNY | |
| 95 | PGVAMPNLYKMQR | M12;M24;M55;M10;M53;M17;M35;M52;M9;M47;M23 |
| | MLLEKCDLQNYG | |
| 96 | TKGTLEPEYFNSVC | M4;M13;M12;M14;M16;M19;M1;M3;M6;M18;M17;M15 |
| | RLMKTIGPDMF | |
| 97 | LLTKGTLEPEYFNSV | M4;M13;M12;M14;M16;M19;M1;M3;M6;M18;M17;M15 |
| | CRLMKTIGPD | |
| 98 | YKTPPIKDFGGFNFS | M4;M32;M28;M14;M31;M1;M34;M29;M3;M33;M27;M35 |
| | QILPDPSKPS | |
| 99 | KTPPIKDFGGFNFSQ | M4;M32;M28;M14;M31;M1;M34;M29;M3;M33;M27;M35 |
| | ILPDPSKPSK | |
| 100 | TVEKGIYQTSNFRV | M31;M1;M34;M30;M3;M37;M50;M51;M41;M8;M39;M23 |
| | QPTESIVRFPN | |
| 101 | SKCVCSVIDLLLDDF | M4;M52;M14;M16;M3;M48;M18;M8;M43;M15;M35;M47 |
| | VEIIKSQDLS | |
| 102 | KCVCSVIDLLLDDF | M4;M52;M14;M16;M3;M48;M18;M8;M43;M15;M35;M47 |
| | VEIIKSQDLSV | |
| 103 | CVCSVIDLLLDDFVE | M4;M52;M14;M16;M3;M48;M18;M8;M43;M15;M35;M47 |
| | IIKSQDLSVV | |
| 104 | SEDAQGMDNLACE | M4;M1;M3;M45;M37;M49;M4;M1;M3;M45;M37;M49 |
| | DLKPVSEEVVEN | |
| 105 | EDAQGMDNLACED | M4;M1;M3;M45;M37;M49;M4;M1;M3;M45;M37;M49 |
| | LKPVSEEVVENP | |
| 106 | DAQGMDNLACEDL | M4;M1;M3;M45;M37;M49;M4;M1;M3;M45;M37;M49 |
| | KPVSEEVVENPT | |
| 107 | YMGTLSYEQFKKG | M4;M16;M1;M3;M37;M52;M4;M16;M1;M3;M37;M52 |
| | VQIPCTCGKQAT | |
| 108 | MYMGTLSYEQFKK | M4;M16;M1;M3;M37;M52;M4;M16;M1;M3;M37;M52 |
| | GVQIPCTCGKQA | |
| 109 | VMYMGTLSYEQFK | M4;M16;M1;M3;M37;M52;M4;M16;M1;M3;M37;M52 |
| | KGVQIPCTCGKQ | |
| 110 | MGTLSYEQFKKGV | M4;M16;M1;M3;M37;M52;M4;M16;M1;M3;M37;M52 |
| | QIPCTCGKQATK | |
| 111 | VQLTSQWLTNIFGT | M4;M28;M31;M3;M45;M59;M4;M28;M31;M3;M45;M59 |
| | VYEKLKPVLDW | |
| 112 | QLTSQWLTNIFGTV | M4;M28;M31;M3;M45;M59;M4;M28;M31;M3;M45;M59 |
| | YEKLKPVLDWL | |
| 113 | NICYTPSKLIEYTDF | M4;M3;M48;M63;M41;M49;M4;M3;M48;M63;M41;M49 |
| | ATSACVLAAE | |
| 114 | KKPASRELKVTFFP | M21;M70;M3;M57;M36;M63;M21;M70;M3;M57;M36;M63 |
| | DLNGDVVAIDY | |
| 115 | YKKPASRELKVTFF | M21;M70;M3;M57;M36;M63;M21;M70;M3;M57;M36;M63 |
| | PDLNGDVVAID | |
| 116 | KPASRELKVTFFPDL | M21;M70;M3;M57;M36;M63;M21;M70;M3;M57;M36;M63 |
| | NGDVVAIDYK | |
| 117 | SGVVTTVMFLARGI | M12;M14;M29;M3;M22;M20;M12;M14;M29;M3;M22;M20 |
| | VFMCVEYCPIF | |
| 118 | VLALLSDLQDLKW | M4;M77;M54;M70;M60;M3;M4;M77;M54;M70;M60;M3 |
| | ARFPKSDGTGTI | |
| 119 | RFVLALLSDLQDLK | M4;M77;M54;M70;M60;M3;M4;M77;M54;M70;M60;M3 |
| | WARFPKSDGTG | |
| 120 | FVLALLSDLQDLKW | M4;M77;M54;M70;M60;M3;M4;M77;M54;M70;M60;M3 |
| | ARFPKSDGTGT | |
| 121 | WPLIVTALRANSAV | M73;M60;M51;M39;M11;M44;M73;M60;M51;M39;M11;M44 |
| | KLQNNELSPVA | |
| 122 | PLIVTALRANSAVK | M73;M60;M51;M39;M11;M44;M73;M60;M51;M39;M11;M44 |
| | LQNNELSPVAL | |
| 123 | AWPLIVTALRANSA | M73;M60;M51;M39;M11;M44;M73;M60;M51;M39;M11;M44 |
| | VKLQNNELSPV | |
| 124 | CSVIDLLLDDFVEIIK | M4;M52;M14;M16;M48;M18;M42;M8;M43;M15;M35;M47 |
| | SQDLSVVSK | |
| 125 | TSLEIPRRNVATLQA | M28;M54;M60;M29;M2;M6;M45;M27;M46;M39;M59;M20 |
| | ENVTGLFKDC | |
| 126 | FTSLEIPRRNVATLQ | M28;M54;M60;M29;M2;M6;M45;M27;M46;M39;M59;M20 |
| | AENVTGLFKD | |
| 127 | QFTSLEIPRRNVATL | M28;M54;M60;M29;M2;M6;M45;M27;M46;M39;M59;M20 |
| | QAENVTGLFK | |
| 128 | SLEIPRRNVATLQAE | M28;M54;M60;M29;M2;M6;M45;M27;M46;M39;M59;M20 |
| | NVTGLFKDCS | |
| 129 | GGFNFSQILPDPSKP | M32;M14;M31;M7;M34;M29;M30;M33;M36;M27;M35;M22 |
| | SKRSFIEDLL | |
| 130 | FLGYFCTCYFGLFCL | M56;M60;M3;M6;M37;M18;M56;M60;M3;M6;M37;M18 |
| | LNRYFRLTLG | |
| 131 | LGYFCTCYFGLFCL | M56;M60;M3;M6;M37;M18;M56;M60;M3;M6;M37;M18 |
| | LNRYFRLTLGV | |
| 132 | IKDFGGFNFSQILPD | M32;M28;M14;M31;M34;M29;M30;M33;M36;M27;M35;M22 |
| | PSKPSKRSFI | |
| 133 | PIKDFGGFNFSQILP | M32;M28;M14;M31;M34;M29;M30;M33;M36;M27;M35;M22 |
| | DPSKPSKRSF | |
| 134 | SILSPLYAFASEAAR | M14;M6;M48;M45;M49;M22;M14;M6;M48;M45;M49;M22 |
| | VVRSIFSRTL | |
| 135 | CNSSTCMMCYKRN | M71;M14;M8;M39;M11;M44;M71;M14;M8;M39;M11;M44 |
| | RATRVECTTIVN | |
| 136 | YNYKLPDDFTGCVI | M13;M32;M12;M14;M55;M29;M38;M48;M67;M68;M49;M26 |
| | AWNSNNLDSKV | |
| 137 | SPLYAFASEAARVV | M14;M6;M48;M45;M49;M59;M14;M6;M48;M45;M49;M59 |
| | RSIFSRTLETA | |
| 138 | FAVHFISNSWLMWL | M11;M73;M3;M50;M9;M23;M11;M73;M3;M50;M9;M23 |
| | IINLVQMAPIS | |
| 139 | YYTSNPTTFHLDGE | M7;M48;M45;M50;M36;M49;M7;M48;M45;M50;M36;M49 |
| | VITFDNLKTLL | |
| 140 | VYYTSNPTTFHLDG | M7;M48;M45;M50;M36;M49;M7;M48;M45;M50;M36;M49 |
| | EVITFDNLKTL | |
| 141 | LPKEITVATSRTLSY | M64;M56;M12;M70;M29;M6;M48;M33;M49;M46;M22;M23 |
| | YKLGASQRVA | |
| 142 | DGCNSSTCMMCYK | M71;M55;M8;M39;M11;M44;M71;M55;M8;M39;M11;M44 |
| | RNRATRVECTTI | |
| 143 | VDGCNSSTCMMCY | M71;M55;M8;M39;M11;M44;M71;M55;M8;M39;M11;M44 |
| | KRNRATRVECTT | |
| 144 | LLLDDFVEIIKSQDL | M4;M52;M16;M48;M18;M42;M8;M43;M15;M35;M47;M23 |
| | SVVSKVVKVT | |
| 145 | LLDDFVEIIKSQDLS | M4;M52;M16;M48;M18;M42;M8;M43;M15;M35;M47;M23 |
| | VVSKVVKVTI | |
| 146 | DLLLDDFVEIIKSQD | M4;M52;M16;M48;M18;M42;M8;M43;M15;M35;M47;M23 |
| | LSVVSKVVKV | |
| 147 | KNNLPFKLTCATTR | M21;M12;M19;M2;M33;M8;M21;M12;M19;M2;M33;M8 |
| | QVVNVVTTKIA | |
| 148 | NNLPFKLTCATTRQ | M21;M12;M19;M2;M33;M8;M21;M12;M19;M2;M33;M8 |
| | VVNVVTTKIAL | |
| 149 | NSTVLSFCAFAVDA | M32;M12;M28;M48;M36;M69;M32;M12;M28;M48;M36;M69 |
| | AKAYKDYLASG | |
| 150 | LYAFASEAARVVRS | M4;M14;M6;M48;M42;M59;M4;M14;M6;M48;M42;M59 |
| | IFSRTLETAQN | |
| 151 | KPPPGDQFKHLIPLM | M56;M19;M12;M16;M54;M25;M34;M60;M18;M15;M39;M47 |
| | YKGLPWNVVR | |
| 152 | ALYYPSARIVYTAC | M64;M21;M28;M14;M5;M29;M38;M67;M17;M49;M46;M22 |
| | SHAAVDALCEK | |
| 153 | YYPSARIVYTACSH | M64;M21;M28;M14;M5;M29;M38;M67;M17;M49;M46;M22 |
| | AAVDALCEKAL | |
| 154 | LALYYPSARIVYTA | M64;M21;M28;M14;M5;M29;M38;M67;M17;M49;M46;M22 |
| | CSHAAVDALCE | |
| 155 | LYYPSARIVYTACS | M64;M21;M28;M14;M5;M29;M38;M67;M17;M49;M46;M22 |
| | HAAVDALCEKA | |
| 156 | FNGECPNFVFPLNSII | M28;M19;M6;M61;M50;M72;M28;M19;M6;M61;M50;M72 |
| | KTIQPRVEK | |
| 157 | TNNAMQVESDDYIA | M19;M10;M29;M18;M11;M44;M19;M10;M29;M18;M11;M44 |
| | TNGPLKVGGSC | |
| 158 | VLGSLAATVRLQAG | M16;M55;M10;M53;M51;M17;M16;M55;M10;M53;M51;M17 |
| | NATEVPANSTV | |
| 159 | MVLGSLAATVRLQ | M16;M55;M10;M53;M51;M17;M16;M55;M10;M53;M51;M17 |
| | AGNATEVPANST | |
| 160 | RSAAKKNNLPFKLT | M19;M12;M78;M25;M33;M8;M19;M12;M78;M25;M33;M8 |
| | CATTRQVVNVV | |
| 161 | IRSAAKKNNLPFKLT | M19;M12;M78;M25;M33;M8;M19;M12;M78;M25;M33;M8 |
| | CATTRQVVNV | |
| 162 | EQKSILSPLYAFASE | M21;M6;M48;M45;M49;M22;M21;M6;M48;M45;M49;M22 |
| | AARVVRSIFS | |
| 163 | NIGEQKSILSPLYAF | M21;M6;M48;M45;M49;M22;M21;M6;M48;M45;M49;M22 |
| | ASEAARVVRS | |
| 164 | IGEQKSILSPLYAFA | M21;M6;M48;M45;M49;M22;M21;M6;M48;M45;M49;M22 |
| | SEAARVVRSI | |
| 165 | GEQKSILSPLYAFAS | M21;M6;M48;M45;M49;M22;M21;M6;M48;M45;M49;M22 |
| | EAARVVRSIF | |
| 166 | NLKTLLSLREVRTIK | M14;M29;M6;M72;M49;M26;M14;M29;M6;M72;M49;M26 |
| | VFTTVDNINL | |
| 167 | LKTLLSLREVRTIKV | M14;M29;M6;M72;M49;M26;M14;M29;M6;M72;M49;M26 |
| | FTTVDNINLH | |
| 168 | REVGFVVPGLPGTIL | M64;M11;M12;M1;M6;M9;M64;M11;M12;M1;M6;M9 |
| | RTTNGDFLHF | |
| 169 | TREVGFVVPGLPGTI | M64;M11;M12;M1;M6;M9;M64;M11;M12;M1;M6;M9 |
| | LRTTNGDFLH | |
| 170 | EVRTIKVFTTVDNIN | M29;M51;M67;M43;M42;M26;M29;M51;M67;M43;M42;M26 |
| | LHTQVVDMSM | |
| 171 | PYFFTLLLQLCTFTR | M19;M25;M33;M8;M42;M39;M19;M25;M33;M8;M42;M39 |
| | STNSRIKASM | |
| 172 | YFFTLLLQLCTFTRS | M19;M25;M33;M8;M42;M39;M19;M25;M33;M8;M42;M39 |
| | TNSRIKASMP | |
| 173 | QLCTFTRSTNSRIKA | M14;M33;M8;M42;M39;M11;M14;M33;M8;M42;M39;M11 |
| | SMPTTIAKNT | |
| 174 | LQLCTFTRSTNSRIK | M14;M33;M8;M42;M39;M11;M14;M33;M8;M42;M39;M11 |
| | ASMPTTIAKN | |
| 175 | LLQLCTFTRSTNSRI | M14;M33;M8;M42;M39;M11;M14;M33;M8;M42;M39;M11 |
| | KASMPTTIAK | |
| 176 | RVSAKPPPGDQFKH | M56;M19;M16;M54;M25;M34;M60;M18;M15;M52;M39;M47 |
| | LIPLMYKGLPW | |
| 177 | AKPPPGDQFKHLIPL | M56;M19;M16;M54;M25;M34;M60;M18;M15;M52;M39;M47 |
| | MYKGLPWNVV | |
| 178 | VSAKPPPGDQFKHLI | M56;M19;M16;M54;M25;M34;M60;M18;M15;M52;M39;M47 |
| | PLMYKGLPWN | |
| 179 | SAKPPPGDQFKHLIP | M56;M19;M16;M54;M25;M34;M60;M18;M15;M52;M39;M47 |
| | LMYKGLPWNV | |
| 180 | KLVNKFLALCADSII | M34;M45;M41;M67;M8;M39;M34;M45;M41;M67;M8;M39 |
| | IGGAKLKALN | |
| 181 | LGTEVNEFACVVAD | M14;M66;M25;M34;M8;M39;M14;M66;M25;M34;M8;M39 |
| | AVIKTLQPVSE | |
| 182 | ELGTEVNEFACVVA | M14;M66;M25;M34;M8;M39;M14;M66;M25;M34;M8;M39 |
| | DAVIKTLQPVS | |
| 183 | TVELGTEVNEFACV | M14;M66;M25;M34;M8;M39;M14;M66;M25;M34;M8;M39 |
| | VADAVIKTLQP | |
| 184 | VELGTEVNEFACVV | M14;M66;M25;M34;M8;M39;M14;M66;M25;M34;M8;M39 |
| | ADAVIKTLQPV | |
| 185 | TLLLQLCTFTRSTNS | M14;M25;M33;M8;M42;M39;M14;M25;M33;M8;M42;M39 |
| | RIKASMPTTI | |
| 186 | MPNMLRIMASLVLA | M56;M21;M12;M5;M30;M69;M41;M35;M46;M39;M22;M23 |
| | RKHTTCCSLSH | |
| 187 | GLNGYTVEEAKTVL | M19;M78;M60;M45;M39;M9;M19;M78;M60;M45;M39;M9 |
| | KKCKSAFYILP | |
| 188 | VPYCYDTNVLEGSV | M12;M14;M5;M34;M29;M46;M12;M14;M5;M34;M29;M46 |
| | AYESLRPDTRY | |
| 189 | NYITTYPGQGLNGY | M75;M70;M45;M63;M76;M39;M75;M70;M45;M63;M76;M39 |
| | TVEEAKTVLKK | |
| 190 | FYVYANGGKGFCK | M45;M41;M42;M8;M43;M39;M45;M41;M42;M8;M43;M39 |
| | LHNWNCVNCDTF | |
| 191 | YANGGKGFCKLHN | M45;M41;M42;M8;M43;M39;M45;M41;M42;M8;M43;M39 |
| | WNCVNCDTFCAG | |
| 192 | ANGGKGFCKLHNW | M45;M41;M42;M8;M43;M39;M45;M41;M42;M8;M43;M39 |
| | NCVNCDTFCAGS | |
| 193 | VYANGGKGFCKLH | M45;M41;M42;M8;M43;M39;M45;M41;M42;M8;M43;M39 |
| | NWNCVNCDTFCA | |
| 194 | YVYANGGKGFCKL | M45;M41;M42;M8;M43;M39;M45;M41;M42;M8;M43;M39 |
| | HNWNCVNCDTFC | |
| 195 | DTANPKTPKYKFVR | M40;M14;M34;M30;M29;M39;M40;M14;M34;M30;M29;M39 |
| | IQPGQTFSVLA | |
| 196 | TANPKTPKYKFVRI | M40;M14;M34;M30;M29;M39;M40;M14;M34;M30;M29;M39 |
| | QPGQTFSVLAC | |
| 197 | ANPKTPKYKFVRIQ | M40;M14;M34;M30;M29;M39;M40;M14;M34;M30;M29;M39 |
| | PGQTFSVLACY | |
| 198 | NPKTPKYKFVRIQP | M40;M14;M34;M30;M29;M39;M40;M14;M34;M30;M29;M39 |
| | GQTFSVLACYN | |
| 199 | YYHTTDPSFLGRYM | M19;M66;M1;M25;M37;M33;M19;M66;M1;M25;M37;M33 |
| | SALNHTKKWKY | |
| 200 | YHTTDPSFLGRYMS | M19;M66;M1;M25;M37;M33;M19;M66;M1;M25;M37;M33 |
| | ALNHTKKWKYP | |
| 201 | LAKALRKVPTDNYI | M14;M29;M48;M49;M15;M26;M14;M29;M48;M49;M15;M26 |
| | TTYPGQGLNGY | |
| 202 | MLAKALRKVPTDN | M14;M29;M48;M49;M15;M26;M14;M29;M48;M49;M15;M26 |
| | YITTYPGQGLNG | |
| 203 | NFVFPLNSIIKTIQPR | M28;M29;M6;M72;M62;M59;M28;M29;M6;M72;M62;M59 |
| | VEKKKLDGF | |
| 204 | VFPLNSIIKTIQPRVE | M28;M29;M6;M72;M62;M59;M28;M29;M6;M72;M62;M59 |
| | KKKLDGFMG | |
| 205 | PNFVFPLNSIIKTIQP | M28;M29;M6;M72;M62;M59;M28;M29;M6;M72;M62;M59 |
| | RVEKKKLDG | |
| 206 | FVFPLNSIIKTIQPRV | M28;M29;M6;M72;M62;M59;M28;M29;M6;M72;M62;M59 |
| | EKKKLDGFM | |
| 207 | FPLNSIIKTIQPRVEK | M28;M29;M6;M72;M62;M59;M28;M29;M6;M72;M62;M59 |
| | KKLDGFMGR | |
| 208 | YCYDTNVLEGSVAY | M12;M14;M5;M34;M18;M46;M12;M14;M5;M34;M18;M46 |
| | ESLRPDTRYVL | |
| 209 | CYDTNVLEGSVAYE | M12;M14;M5;M34;M18;M46;M12;M14;M5;M34;M18;M46 |
| | SLRPDTRYVLM | |
| 210 | VKDFMSLSEQLRKQ | M56;M19;M66;M25;M6;M18;M56;M19;M66;M25;M6;M18 |
| | IRSAAKKNNLP | |
| 211 | DNLACEDLKPVSEE | M73;M45;M37;M50;M49;M44;M73;M45;M37;M50;M49;M44 |
| | VVENPTIQKDV | |
| 212 | MDNLACEDLKPVSE | M73;M45;M37;M50;M49;M44;M73;M45;M37;M50;M49;M44 |
| | EVVENPTIQKD | |
| 213 | DTNVLEGSVAYESL | M12;M19;M34;M50;M18;M46;M12;M19;M34;M50;M18;M46 |
| | RPDTRYVLMDG | |
| 214 | ASKIITLKKRWQLA | M9;M58;M24;M55;M60;M74;M33;M27;M8;M17;M42;M82 |
| | LSKGVHFVCNL | |
| 215 | SDVLLPLTQYNRYL | M19;M14;M53;M69;M27;M20;M19;M14;M53;M69;M27;M20 |
| | ALYNKYKYFSG | |
| 216 | VLEGSVAYESLRPD | M19;M24;M7;M34;M50;M18;M19;M24;M7;M34;M50;M18 |
| | TRYVLMDGSII | |
| 217 | LEGSVAYESLRPDT | M19;M24;M7;M34;M50;M18;M19;M24;M7;M34;M50;M18 |
| | RYVLMDGSIIQ | |
| 218 | EDLLIRKSNHNFLV | M12;M24;M14;M70;M10;M9;M12;M24;M14;M70;M10;M9 |
| | QAGNVQLRVIG | |
| 219 | NYEDLLIRKSNHNF | M12;M24;M14;M70;M10;M9;M12;M24;M14;M70;M10;M9 |
| | LVQAGNVQLRV | |
| 220 | YEDLLIRKSNHNFL | M12;M24;M14;M70;M10;M9;M12;M24;M14;M70;M10;M9 |
| | VQAGNVQLRVI | |
| 221 | IPGIPKDMTYRRLIS | M40;M58;M16;M31;M25;M34;M30;M43;M15;M42;M39;M47 |
| | MMGFKMNYQV | |
| 222 | VDIPGIPKDMTYRRL | M40;M58;M16;M31;M25;M34;M30;M43;M15;M42;M39;M47 |
| | ISMMGFKMNY | |
| 223 | DIPGIPKDMTYRRLI | M40;M58;M16;M31;M25;M34;M30;M43;M15;M42;M39;M47 |
| | SMMGFKMNYQ | |
| 224 | ADDLNQLTGYKKP | M58;M19;M70;M53;M63;M15;M58;M19;M70;M53;M63;M15 |
| | ASRELKVTFFPD | |
| 225 | AFEYYHTTDPSFLG | M1;M25;M29;M6;M37;M33;M1;M25;M29;M6;M37;M33 |
| | RYMSALNHTKK | |
| 226 | APNMMVTNNTFTL | M11;M12;M24;M10;M6;M9;M11;M12;M24;M10;M6;M9 |
| | KGGAPTKVTFGD | |
| 227 | PNLAWPLIVTALRA | M73;M60;M46;M39;M11;M44;M73;M60;M46;M39;M11;M44 |
| | NSAVKLQNNEL | |
| 228 | SPNLAWPLIVTALR | M73;M60;M46;M39;M11;M44;M73;M60;M46;M39;M11;M44 |
| | ANSAVKLQNNE | |
| 229 | NLAWPLIVTALRAN | M73;M60;M46;M39;M11;M44;M73;M60;M46;M39;M11;M44 |
| | SAVKLQNNELS | |
| 230 | EAFEYYHTTDPSFL | M1;M29;M6;M37;M33;M41;M1;M29;M6;M37;M33;M41 |
| | GRYMSALNHTK | |
| 231 | TTEVVGDIILKPANN | M28;M58;M19;M34;M53;M26;M28;M58;M19;M34;M53;M26 |
| | SLKITEEVGH | |
| 232 | EVVGDIILKPANNSL | M28;M58;M19;M34;M53;M26;M28;M58;M19;M34;M53;M26 |
| | KITEEVGHTD | |
| 233 | VVGDIILKPANNSLK | M28;M58;M19;M34;M53;M26;M28;M58;M19;M34;M53;M26 |
| | ITEEVGHTDL | |
| 234 | TEVVGDIILKPANNS | M28;M58;M19;M34;M53;M26;M28;M58;M19;M34;M53;M26 |
| | LKITEEVGHT | |
| 235 | KEGVEFLRDGWEIV | M4;M21;M6;M74;M49;M22;M4;M21;M6;M74;M49;M22 |
| | KFISTCACEIV | |
| 236 | WAKRNIKPVPEVKI | M9;M54;M66;M60;M74;M48;M57;M27;M43;M49;M42;M22 |
| | LNNLGVDIAAN | |
| 237 | MDNSPNLAWPLIVT | M73;M60;M18;M39;M11;M44;M73;M60;M18;M39;M11;M44 |
| | ALRANSAVKLQ | |
| 238 | KTPKYKFVRIQPGQ | M40;M14;M34;M29;M27;M39;M40;M14;M34;M29;M27;M39 |
| | TFSVLACYNGS | |
| 239 | PKTPKYKFVRIQPG | M40;M14;M34;M29;M27;M39;M40;M14;M34;M29;M27;M39 |
| | QTFSVLACYNG | |
| 240 | TSGRWVLNNDYYR | M34;M33;M43;M42;M39;M44;M34;M33;M43;M42;M39;M44 |
| | SLPGVFCGVDAV | |
| 241 | SGRWVLNNDYYRS | M34;M33;M43;M42;M39;M44;M34;M33;M43;M42;M39;M44 |
| | LPGVFCGVDAVN | |
| 242 | TSNSFDVLKSEDAQ | M64;M6;M41;M46;M39;M22;M64;M6;M41;M46;M39;M22 |
| | GMDNLACEDLK | |
| 243 | GLNNLNRGMVLGS | M12;M16;M55;M10;M53;M17;M12;M16;M55;M10;M53;M17 |
| | LAATVRLQAGNA | |
| 244 | VRQCSGVTFQSAVK | M71;M19;M25;M33;M26;M9;M71;M19;M25;M33;M26;M9 |
| | RTIKGTHHWLL | |
| 245 | PLTQYNRYLALYNK | M19;M55;M67;M27;M42;M20;M19;M55;M67;M27;M42;M20 |
| | YKYFSGAMDTT | |
| 246 | KLNEEIAIILASFSAS | M28;M38;M33;M43;M8;M42;M28;M38;M33;M43;M8;M42 |
| | TSAFVETVK | |
| 247 | ADQAMTQMYKQAR | M56;M19;M55;M25;M74;M18;M56;M19;M55;M25;M74;M18 |
| | SEDKRAKVTSAM | |
| 248 | MADQAMTQMYKQ | M56;M19;M55;M25;M74;M18;M56;M19;M55;M25;M74;M18 |
| | ARSEDKRAKVTSA | |
| 249 | KMADQAMTQMYK | M56;M19;M55;M25;M74;M18;M56;M19;M55;M25;M74;M18 |
| | QARSEDKRAKVTS | |
| 250 | LWSTKPVETSNSFD | M64;M25;M6;M41;M39;M22;M64;M25;M6;M41;M39;M22 |
| | VLKSEDAQGMD | |
| 251 | CVPLNIIPLTTAAKL | M14;M24;M38;M82;M57;M22;M14;M24;M38;M82;M57;M22 |
| | MVVIPDYNTY | |
| 252 | AVANGDSEVVLKK | M71;M13;M55;M1;M53;M17;M71;M13;M55;M1;M53;M17 |
| | LKKSLNVAKSEF | |
| 253 | VANGDSEVVLKKL | M71;M13;M55;M1;M53;M17;M71;M13;M55;M1;M53;M17 |
| | KKSLNVAKSEFD | |
| 254 | LNNDYYRSLPGVFC | M25;M34;M45;M33;M43;M39;M25;M34;M45;M33;M43;M39 |
| | GVDAVNLLTNM | |
| 255 | KIALKGGKIVNNWL | M56;M12;M55;M57;M11;M20;M56;M12;M55;M57;M11;M20 |
| | KQLIKVTLVFL | |
| 256 | TKIALKGGKIVNNW | M56;M12;M55;M57;M11;M20;M56;M12;M55;M57;M11;M20 |
| | LKQLIKVTLVF | |
| 257 | IALKGGKIVNNWLK | M56;M12;M55;M57;M11;M20;M56;M12;M55;M57;M11;M20 |
| | QLIKVTLVFLF | |
| 258 | TTKIALKGGKIVNN | M56;M12;M55;M57;M11;M20;M56;M12;M55;M57;M11;M20 |
| | WLKQLIKVTLV | |
| 259 | PCSVCLSGLDSLDT | M21;M55;M34;M48;M41;M22;M21;M55;M34;M48;M41;M22 |
| | YPSLETIQITI | |
| 260 | IPCSVCLSGLDSLDT | M21;M55;M34;M48;M41;M22;M21;M55;M34;M48;M41;M22 |
| | YPSLETIQIT | |
| 261 | CSVCLSGLDSLDTY | M21;M55;M34;M48;M41;M22;M21;M55;M34;M48;M41;M22 |
| | PSLETIQITIS | |
| 262 | SVCLSGLDSLDTYPS | M21;M55;M34;M48;M41;M22;M21;M55;M34;M48;M41;M22 |
| | LETIQITISS | |
| 263 | SIPCSVCLSGLDSLD | M21;M55;M34;M48;M41;M22;M21;M55;M34;M48;M41;M22 |
| | TYPSLETIQI | |
| 264 | GSIPCSVCLSGLDSL | M21;M55;M34;M48;M41;M22;M21;M55;M34;M48;M41;M22 |
| | DTYPSLETIQ | |
| 265 | VCLSGLDSLDTYPSL | M21;M55;M34;M48;M41;M22;M21;M55;M34;M48;M41;M22 |
| | ETIQITISSF | |
| 266 | WVLNNDYYRSLPG | M34;M45;M33;M43;M42;M39;M34;M45;M33;M43;M42;M39 |
| | VFCGVDAVNLLT | |
| 267 | KLWAQCVQLHNDIL | M21;M32;M58;M36;M68;M22;M21;M32;M58;M36;M68;M22 |
| | LAKDTTEAFEK | |
| 268 | LWAQCVQLHNDILL | M21;M32;M58;M36;M68;M22;M21;M32;M58;M36;M68;M22 |
| | AKDTTEAFEKM | |
| 269 | AQCVQLHNDILLAK | M21;M32;M58;M36;M68;M22;M21;M32;M58;M36;M68;M22 |
| | DTTEAFEKMVS | |
| 270 | WAQCVQLHNDILLA | M21;M32;M58;M36;M68;M22;M21;M32;M58;M36;M68;M22 |
| | KDTTEAFEKMV | |
| 271 | TVSVSSPDAVTAYN | M55;M48;M41;M49;M35;M26;M55;M48;M41;M49;M35;M26 |
| | GYLTSSSKTPE | |
| 272 | ALIISVTSNYSGVVT | M56;M14;M29;M38;M68;M11;M56;M14;M29;M38;M68;M11 |
| | TVMFLARGIV | |
| 273 | ILMTARTVYDDGAR | M5;M6;M72;M11;M59;M20;M5;M6;M72;M11;M59;M20 |
| | RVWTLMNVLTL | |
| 274 | LKKSLNVAKSEFDR | M32;M28;M7;M50;M33;M68;M32;M28;M7;M50;M33;M68 |
| | DAAMQRKLEKM | |
| 275 | NFKFVCDNIKFADD | M40;M64;M21;M19;M22;M23;M40;M64;M21;M19;M22;M23 |
| | LNQLTGYKKPA | |
| 276 | FKFVCDNIKFADDL | M40;M64;M21;M19;M22;M23;M40;M64;M21;M19;M22;M23 |
| | NQLTGYKKPAS | |
| 277 | EQAVANGDSEVVL | M71;M13;M55;M1;M53;M59;M71;M13;M55;M1;M53;M59 |
| | KKLKKSLNVAKS | |
| 278 | VTLVFLFVAAIFYLI | M56;M32;M1;M34;M18;M39;M56;M32;M1;M34;M18;M39 |
| | TPVHVMSKHT | |
| 279 | TLVFLFVAAIFYLITP | M56;M32;M1;M34;M18;M39;M56;M32;M1;M34;M18;M39 |
| | VHVMSKHTD | |
| 280 | QIVTCAKEIKESVQT | M24;M53;M33;M17;M8;M43;M24;M53;M33;M17;M8;M43 |
| | FFKLVNKFLA | |
| 281 | IVTCAKEIKESVQTF | M24;M53;M33;M17;M8;M43;M24;M53;M33;M17;M8;M43 |
| | FKLVNKFLAL | |
| 282 | TGDSCNNYMLTYN | M53;M8;M15;M52;M11;M47;M53;M8;M15;M52;M11;M47 |
| | KVENMTPRDLGA | |
| 283 | VTGDSCNNYMLTY | M53;M8;M15;M52;M11;M47;M53;M8;M15;M52;M11;M47 |
| | NKVENMTPRDLG | |
| 284 | EVTGDSCNNYMLT | M53;M8;M15;M52;M11;M47;M53;M8;M15;M52;M11;M47 |
| | YNKVENMTPRDL | |
| 285 | IEVTGDSCNNYMLT | M53;M8;M15;M52;M11;M47;M53;M8;M15;M52;M11;M47 |
| | YNKVENMTPRD | |
| 286 | GSVGFNIDYDCVSF | M14;M18;M17;M43;M52;M9;M14;M18;M17;M43;M52;M9 |
| | CYMHHMELPTG | |
| 287 | KYCALAPNMMVTN | M11;M24;M10;M6;M50;M9;M11;M24;M10;M6;M50;M9 |
| | NTFTLKGGAPTK | |
| 288 | EKYCALAPNMMVT | M11;M24;M10;M6;M50;M9;M11;M24;M10;M6;M50;M9 |
| | NNTFTLKGGAPT | |
| 289 | TEKYCALAPNMMV | M11;M24;M10;M6;M50;M9;M11;M24;M10;M6;M50;M9 |
| | TNNTFTLKGGAP | |
| 290 | YCALAPNMMVTNN | M11;M24;M10;M6;M50;M9;M11;M24;M10;M6;M50;M9 |
| | TFTLKGGAPTKV | |
| 291 | QRKLEKMADQAMT | M56;M55;M2;M74;M18;M82;M56;M55;M2;M74;M18;M82 |
| | QMYKQARSEDKR | |
| 292 | MQRKLEKMADQA | M56;M55;M2;M74;M18;M82;M56;M55;M2;M74;M18;M82 |
| | MTQMYKQARSEDK | |
| 293 | GQIVTCAKEIKESVQ | M65;M53;M79;M33;M8;M23;M65;M53;M79;M33;M8;M23 |
| | TFFKLVNKFL | |
| 294 | NPKGFCDLKGKYV | M40;M31;M30;M43;M42;M39;M40;M31;M30;M43;M42;M39 |
| | QIPTTCANDPVG | |
| 295 | DGSIIQFPNTYLEGS | M12;M43;M42;M26;M22;M20;M12;M43;M42;M26;M22;M20 |
| | VRVVTTFDSE | |
| 296 | QPQLEMELTPVVQT | M56;M60;M29;M6;M50;M59;M56;M60;M29;M6;M50;M59 |
| | IEVNSFSGYLK | |
| 297 | QLEMELTPVVQTIE | M56;M60;M29;M6;M50;M59;M56;M60;M29;M6;M50;M59 |
| | VNSFSGYLKLT | |
| 298 | LEMELTPVVQTIEV | M56;M60;M29;M6;M50;M59;M56;M60;M29;M6;M50;M59 |
| | NSFSGYLKLTD | |
| 299 | PQLEMELTPVVQTIE | M56;M60;M29;M6;M50;M59;M56;M60;M29;M6;M50;M59 |
| | VNSFSGYLKL | |
| 300 | DGARRVWTLMNVL | M24;M5;M2;M18;M22;M20;M24;M5;M2;M18;M22;M20 |
| | TLVYKVYYGNAL | |
| 301 | GPEHSLAEYHNESG | M13;M58;M16;M53;M17;M39;M13;M58;M16;M53;M17;M39 |
| | LKTILRKGGRT | |
| 302 | SLAEYHNESGLKTIL | M13;M58;M16;M53;M17;M39;M13;M58;M16;M53;M17;M39 |
| | RKGGRTIAFG | |
| 303 | PEHSLAEYHNESGL | M13;M58;M16;M53;M17;M39;M13;M58;M16;M53;M17;M39 |
| | KTILRKGGRTI | |
| 304 | EHSLAEYHNESGLK | M13;M58;M16;M53;M17;M39;M13;M58;M16;M53;M17;M39 |
| | TILRKGGRTIA | |
| 305 | HSLAEYHNESGLKTI | M13;M58;M16;M53;M17;M39;M13;M58;M16;M53;M17;M39 |
| | LRKGGRTIAF | |
| 306 | RSDVLLPLTQYNRY | M14;M53;M61;M69;M27;M20;M14;M53;M61;M69;M27;M20 |
| | LALYNKYKYFS | |
| 307 | ASFYYVWKSYVHV | M24;M1;M34;M37;M35;M23;M24;M1;M34;M37;M35;M23 |
| | VDGCNSSTCMMC | |
| 308 | RSIFSRTLETAQNSV | M54;M24;M5;M34;M2;M9;M54;M24;M5;M34;M2;M9 |
| | RVLQKAAITI | |
| 309 | SIFSRTLETAQNSVR | M54;M24;M5;M34;M2;M9;M54;M24;M5;M34;M2;M9 |
| | VLQKAAITIL | |
| 310 | SIIQFPNTYLEGSVR | M64;M12;M27;M20;M22;M23;M64;M12;M27;M20;M22;M23 |
| | VVTTFDSEYC | |
| 311 | DSCKRVLNVVCKTC | M58;M5;M2;M27;M15;M52;M58;M5;M2;M27;M15;M52 |
| | GQQQTTLKGVE | |
| 312 | TEAFEKMVSLLSVL | M61;M74;M69;M67;M39;M22;M61;M74;M69;M67;M39;M22 |
| | LSMQGAVDINK | |
| 313 | PSIISNEKQEILGTVS | M75;M56;M24;M8;M9;M23;M75;M56;M24;M8;M9;M23 |
| | WNLREMLAH | |
| 314 | ETVKGLDYKAFKQI | M1;M37;M42;M52;M47;M23;M1;M37;M42;M52;M47;M23 |
| | VESCGNFKVTK | |
| 315 | TVKGLDYKAFKQIV | M1;M37;M42;M52;M47;M23;M1;M37;M42;M52;M47;M23 |
| | ESCGNFKVTKG | |
| 316 | QHLKDGTCGLVEVE | M13;M28;M24;M66;M74;M9;M13;M28;M24;M66;M74;M9 |
| | KGVLPQLEQPY | |
| 317 | LKDGTCGLVEVEKG | M13;M28;M24;M66;M74;M9;M13;M28;M24;M66;M74;M9 |
| | VLPQLEQPYVF | |
| 318 | HLKDGTCGLVEVEK | M13;M28;M24;M66;M74;M9;M13;M28;M24;M66;M74;M9 |
| | GVLPQLEQPYV | |
| 319 | TVYEKLKPVLDWLE | M32;M28;M61;M67;M9;M20;M32;M28;M61;M67;M9;M20 |
| | EKFKEGVEFLR | |
| 320 | IANYAKPFLNKVVS | M78;M10;M8;M15;M9;M23;M78;M10;M8;M15;M9;M23 |
| | TTTNIVTRCLN | |
| 321 | KPFLNKVVSTTTNIV | M78;M10;M37;M8;M15;M23;M78;M10;M37;M8;M15;M23 |
| | TRCLNRVCTN | |
| 322 | GTVYEKLKPVLDW | M32;M28;M31;M67;M9;M20;M32;M28;M31;M67;M9;M20 |
| | LEEKFKEGVEFL | |
| 323 | NRATLQAIASEFSSL | M21;M41;M42;M43;M39;M22;M21;M41;M42;M43;M39;M22 |
| | PSYAAFATAQ | |
| 324 | AKLKALNLGETFVT | M71;M24;M55;M50;M18;M27;M71;M24;M55;M50;M18;M27 |
| | HSKGLYRKCVK | |
| 325 | TIKGTHHWLLLTILT | M52;M34;M42;M43;M46;M39;M52;M34;M42;M43;M46;M39 |
| | SLLVLVQSTQ | |
| 326 | IKGTHHWLLLTILTS | M52;M34;M42;M43;M46;M39;M52;M34;M42;M43;M46;M39 |
| | LLVLVQSTQW | |
| 327 | RTIKGTHHWLLLTIL | M52;M34;M42;M43;M46;M39;M52;M34;M42;M43;M46;M39 |
| | TSLLVLVQST | |
| 328 | STTTNIVTRCLNRVC | M64;M78;M6;M37;M69;M20;M64;M78;M6;M37;M69;M20 |
| | TNYMPYFFTL | |
| 329 | YLAVFDKNLYDKL | M70;M41;M42;M49;M43;M39;M70;M41;M42;M49;M43;M39 |
| | VSSFLEMKSEKQ | |
| 330 | LAVFDKNLYDKLVS | M70;M41;M42;M49;M43;M39;M70;M41;M42;M49;M43;M39 |
| | SFLEMKSEKQV | |
| 331 | YFNMVYMPASWV | M32;M52;M83;M15;M46;M39;M32;M52;M83;M15;M46;M39 |
| | MRIMTWLDMVDTS | |
| 332 | NVNKGEDIQLLKSA | M32;M28;M31;M60;M38;M68;M32;M28;M31;M60;M38;M68 |
| | YENFNQHEVLL | |
| 333 | VNKGEDIQLLKSAY | M32;M28;M31;M60;M38;M68;M32;M28;M31;M60;M38;M68 |
| | ENFNQHEVLLA | |
| 334 | PNVNKGEDIQLLKS | M32;M28;M31;M60;M38;M68;M32;M28;M31;M60;M38;M68 |
| | AYENFNQHEVL | |
| 335 | GPNVNKGEDIQLLK | M32;M28;M31;M60;M38;M68;M32;M28;M31;M60;M38;M68 |
| | SAYENFNQHEV | |
| 336 | GETFVTHSKGLYRK | M24;M55;M18;M35;M52;M47;M24;M55;M18;M35;M52;M47 |
| | CVKSREETGLL | |
| 337 | KMSDVKCTSVVLLS | M40;M56;M54;M60;M29;M22;M40;M56;M54;M60;M29;M22 |
| | VLQQLRVESSS | |
| 338 | WLMWLIINLVQMA | M11;M73;M55;M50;M9;M23;M11;M73;M55;M50;M9;M23 |
| | PISAMVRMYIFF | |
| 339 | LMWLIINLVQMAPIS | M11;M73;M55;M50;M9;M23;M11;M73;M55;M50;M9;M23 |
| | AMVRMYIFFA | |
| 340 | AMQTMLFTMLRKL | M16;M43;M15;M52;M9;M47;M16;M43;M15;M52;M9;M47 |
| | DNDALNNIINNA | |
| 341 | SFLGRYMSALNHTK | M24;M5;M25;M61;M74;M33;M24;M5;M25;M61;M74;M33 |
| | KWKYPQVNGLT | |
| 342 | APISAMVRMYIFFAS | M24;M55;M20;M35;M9;M23;M24;M55;M20;M35;M9;M23 |
| | FYYVWKSYVH | |
| 343 | MLCTHTGTGQAITV | M24;M78;M34;M29;M49;M9;M24;M78;M34;M29;M49;M9 |
| | TPEANMDQESF | |
| 344 | THTGTGQAITVTPE | M24;M78;M34;M29;M49;M9;M24;M78;M34;M29;M49;M9 |
| | ANMDQESFGGA | |
| 345 | HTGTGQAITVTPEA | M24;M78;M34;M29;M49;M9;M24;M78;M34;M29;M49;M9 |
| | NMDQESFGGAS | |
| 346 | LCTHTGTGQAITVTP | M24;M78;M34;M29;M49;M9;M24;M78;M34;M29;M49;M9 |
| | EANMDQESFG | |
| 347 | CTHTGTGQAITVTPE | M24;M78;M34;M29;M49;M9;M24;M78;M34;M29;M49;M9 |
| | ANMDQESFGG | |
| 348 | SDVKCTSVVLLSVL | M40;M56;M54;M60;M34;M22;M40;M56;M54;M60;M34;M22 |
| | QQLRVESSSKL | |
| 349 | MPASWVMRIMTWL | M64;M70;M83;M15;M52;M22;M64;M70;M83;M15;M52;M22 |
| | DMVDTSLSGFKL | |
| 350 | EQLDFIDTKRGVYC | M64;M75;M66;M74;M27;M22;M64;M75;M66;M74;M27;M22 |
| | CREHEHEIAWY | |
| 351 | SEQLDFIDTKRGVY | M64;M75;M66;M74;M27;M22;M64;M75;M66;M74;M27;M22 |
| | CCREHEHEIAW | |
| 352 | VLKTGDLQPLEQPT | M54;M58;M60;M38;M17;M15;M54;M58;M60;M38;M17;M15 |
| | SEAVEAPLVGT | |
| 353 | KTGDLQPLEQPTSE | M54;M58;M60;M38;M17;M15;M54;M58;M60;M38;M17;M15 |
| | AVEAPLVGTPV | |
| 354 | EVVLKTGDLQPLEQ | M54;M58;M60;M38;M17;M15;M54;M58;M60;M38;M17;M15 |
| | PTSEAVEAPLV | |
| 355 | LKTGDLQPLEQPTSE | M54;M58;M60;M38;M17;M15;M54;M58;M60;M38;M17;M15 |
| | AVEAPLVGTP | |
| 356 | VVLKTGDLQPLEQP | M54;M58;M60;M38;M17;M15;M54;M58;M60;M38;M17;M15 |
| | TSEAVEAPLVG | |
| 357 | GGQPITNCVKMLCT | M64;M21;M58;M53;M22;M23;M64;M21;M58;M53;M22;M23 |
| | HTGTGQAITVT | |
| 358 | EDMLNPNYEDLLIR | M21;M24;M10;M29;M9;M23;M21;M24;M10;M29;M9;M23 |
| | KSNHNFLVQAG | |
| 359 | KNTVKSVGKFCLEA | M54;M60;M25;M41;M43;M42;M54;M60;M25;M41;M43;M42 |
| | SFNYLKSPNFS | |
| 360 | TLETAQNSVRVLQK | M56;M54;M24;M5;M60;M9;M56;M54;M24;M5;M60;M9 |
| | AAITILDGISQ | |
| 361 | LETAQNSVRVLQKA | M56;M54;M24;M5;M60;M9;M56;M54;M24;M5;M60;M9 |
| | AITILDGISQY | |
| 362 | TEVLTEEVVLKTGD | M54;M58;M60;M38;M17;M62;M54;M58;M60;M38;M17;M62 |
| | LQPLEQPTSEA | |
| 363 | EVLTEEVVLKTGDL | M54;M58;M60;M38;M17;M62;M54;M58;M60;M38;M17;M62 |
| | QPLEQPTSEAV | |
| 364 | KASCTLSEQLDFIDT | M64;M75;M74;M83;M27;M22;M64;M75;M74;M83;M27;M22 |
| | KRGVYCCREH | |
| 365 | ASCTLSEQLDFIDTK | M64;M75;M74;M83;M27;M22;M64;M75;M74;M83;M27;M22 |
| | RGVYCCREHE | |
| 366 | EEAKKVKPTVVVN | M56;M73;M50;M18;M11;M23;M56;M73;M50;M18;M11;M23 |
| | AANVYLKHGGGV | |
| 367 | EAKKVKPTVVVNA | M56;M73;M50;M18;M11;M23;M56;M73;M50;M18;M11;M23 |
| | ANVYLKHGGGVA | |
| 368 | VEEAKKVKPTVVV | M56;M73;M50;M18;M11;M23;M56;M73;M50;M18;M11;M23 |
| | NAANVYLKHGGG | |
| 369 | VYEKLKPVLDWLEE | M32;M61;M67;M27;M9;M20;M32;M61;M67;M27;M9;M20 |
| | KFKEGVEFLRD | |
| 370 | GNALDQAISMWALI | M64;M56;M32;M68;M22;M23;M64;M56;M32;M68;M22;M23 |
| | ISVTSNYSGVV | |
| 371 | NALDQAISMWALIIS | M64;M56;M32;M68;M22;M23;M64;M56;M32;M68;M22;M23 |
| | VTSNYSGVVT | |
| 372 | KPTVVVNAANVYL | M56;M11;M73;M18;M9;M23;M56;M11;M73;M18;M9;M23 |
| | KHGGGVAGALNK | |
| 373 | VLDMCASLKELLQN | M56;M24;M60;M50;M18;M59;M56;M24;M60;M50;M18;M59 |
| | GMNGRTILGSA | |
| 374 | AYYNTTKGGRFVLA | M32;M70;M31;M33;M68;M20;M32;M70;M31;M33;M68;M20 |
| | LLSDLQDLKWA | |
| 375 | KGGRFVLALLSDLQ | M77;M32;M54;M70;M60;M20;M77;M32;M54;M70;M60;M20 |
| | DLKWARFPKSD | |
| 376 | SAMVRMYIFFASFY | M24;M37;M20;M35;M9;M23;M24;M37;M20;M35;M9;M23 |
| | YVWKSYVHVVD | |
| 377 | IAVLDMCASLKELL | M56;M32;M24;M10;M60;M59;M56;M32;M24;M10;M60;M59 |
| | QNGMNGRTILG | |
| 378 | GIAVLDMCASLKEL | M56;M32;M24;M10;M60;M59;M56;M32;M24;M10;M60;M59 |
| | LQNGMNGRTIL | |
| 379 | VQTIEVNSFSGYLKL | M56;M38;M50;M9;M47;M23;M56;M38;M50;M9;M47;M23 |
| | TDNVYIKNAD | |
| 380 | VDLPIGINITRFQTLL | M4;M23;M21;M19;M24;M5;M1;M25;M3;M2;M26;M22;M20 |
| | ALHRSYLTP | |
| 381 | LPIGINITRFQTLLAL | M4;M23;M21;M19;M24;M55;M5;M25;M3;M2;M26;M22;M20 |
| | HRSYLTPGD | |
| 382 | DLPIGINITRFQTLLA | M4;M23;M21;M19;M24;M55;M5;M25;M3;M2;M26;M22;M20 |
| | LHRSYLTPG | |
| 383 | VCSVIDLLLDDFVEII | M4;M52;M14;M16;M3;M48;M18;M42;M8;M43;M15;M35;M47 |
| | KSQDLSVVS | |
| 384 | LSFKELLVYAADPA | M64;M30;M48;M57;M33;M36;M41;M8;M49;M26;M22;M44;M20 |
| | MHAASGNLLLD | |
| 385 | SRLSFKELLVYAAD | M64;M30;M48;M57;M33;M36;M41;M8;M49;M26;M22;M44;M20 |
| | PAMHAASGNLL | |
| 386 | RLSFKELLVYAADP | M64;M30;M48;M57;M33;M36;M41;M8;M49;M26;M22;M44;M20 |
| | AMHAASGNLLL | |
| 387 | CPAEIVDTVSALVY | M12;M28;M16;M7;M29;M6;M38;M50;M8;M17;M35;M59;M23 |
| | DNKLKAHKDKS | |
| 388 | DFGGFNFSQILPDPS | M32;M28;M14;M31;M7;M34;M29;M30;M33;M36;M27;M35;M22 |
| | KPSKRSFIED | |
| 389 | KDFGGFNFSQILPDP | M32;M28;M14;M31;M7;M34;M29;M30;M33;M36;M27;M35;M22 |
| | SKPSKRSFIE | |
| 390 | FGGFNFSQILPDPSK | M32;M28;M14;M31;M7;M34;M29;M30;M33;M36;M27;M35;M22 |
| | PSKRSFIEDL | |
| 391 | LEIPRRNVATLQAE | M28;M54;M60;M29;M2;M6;M45;M38;M27;M46;M39;M59;M20 |
| | NVTGLFKDCSK | |
| 392 | IDLLLDDFVEIIKSQ | M4;M52;M14;M16;M48;M18;M42;M8;M43;M15;M35;M47;M23 |
| | DLSVVSKVVK | |
| 393 | SVIDLLLDDFVEIIKS | M4;M52;M14;M16;M48;M18;M42;M8;M43;M15;M35;M47;M23 |
| | QDLSVVSKV | |
| 394 | VIDLLLDDFVEIIKS | M4;M52;M14;M16;M48;M18;M42;M8;M43;M15;M35;M47;M23 |
| | QDLSVVSKVV | |
| 395 | YKQFDTYNLWNTFT | M71;M58;M14;M16;M54;M25;M34;M53;M60;M19;M17;M15;M39 |
| | RLQSLENVAFN | |
| 396 | DTYNLWNTFTRLQS | M71;M58;M14;M16;M54;M25;M34;M60;M19;M17;M15;M42;M39 |
| | LENVAFNVVNK | |
| 397 | DLPKEITVATSRTLS | M64;M56;M12;M70;M55;M29;M6;M48;M33;M49;M46;M22;M23 |
| | YYKLGASQRV | |
| 398 | MADSNGTITVEELK | M4;M56;M28;M58;M16;M55;M80;M53;M6;M17;M52;M47;M59 |
| | KLLEQWNLVIG | |
| 399 | IGINITRFQTLLALHR | M23;M21;M9;M19;M24;M55;M5;M10;M25;M2;M48;M22;M20 |
| | SYLTPGDSS | |
| 400 | GINITRFQTLLALHR | M23;M21;M9;M19;M24;M55;M5;M10;M25;M2;M48;M22;M20 |
| | SYLTPGDSSS | |
| 401 | YPSARIVYTACSHA | M64;M21;M28;M14;M5;M60;M29;M38;M67;M17;M49;M46;M22 |
| | AVDALCEKALK | |
| 402 | AMPNMLRIMASLVL | M56;M21;M12;M25;M5;M30;M69;M41;M35;M46;M39;M22;M23 |
| | ARKHTTCCSLS | |
| 403 | GIPKDMTYRRLISM | M40;M58;M16;M31;M25;M34;M30;M46;M43;M15;M42;M39;M47 |
| | MGFKMNYQVNG | |
| 404 | PGIPKDMTYRRLISM | M40;M58;M16;M31;M25;M34;M30;M46;M43;M15;M42;M39;M47 |
| | MGFKMNYQVN | |
| 405 | ALLAVFQSASKIITL | M56;M9;M58;M24;M16;M55;M60;M53;M61;M17;M15;M52;M82 |
| | KKRWQLALSK | |
| 406 | LLAVFQSASKIITLK | M56;M9;M58;M24;M16;M55;M60;M53;M27;M17;M15;M52;M82 |
| | KRWQLALSKG | |
| 407 | VALLAVFQSASKIIT | M9;M58;M24;M55;M60;M53;M61;M17;M35;M15;M52;M26;M82 |
| | LKKRWQLALS | |
| 408 | PPLNRNYVFTGYRV | M4;M40;M24;M31;M1;M38;M3;M37;M41;M8;M43;M42;M39;M44 |
| | TKNSKVQIGEY | |
| 409 | RPPLNRNYVFTGYR | M4;M40;M24;M31;M1;M38;M3;M37;M41;M8;M43;M42;M39;M44 |
| | VTKNSKVQIGE | |
| 410 | PIGINITRFQTLLALH | M4;M23;M21;M9;M19;M24;M55;M5;M10;M25;M3;M2;M22;M20 |
| | RSYLTPGDS | |
| 411 | AVMYMGTLSYEQF | M4;M16;M1;M3;M6;M37;M52;M4;M16;M1;M3;M6;M37;M52 |
| | KKGVQIPCTCGK | |
| 412 | KHGTFTCASEYTGN | M4;M66;M1;M65;M3;M38;M33;M4;M66;M1;M65;M3;M38;M33 |
| | YQCGHYKHITS | |
| 413 | HGTFTCASEYTGNY | M4;M1;M65;M3;M38;M33;M42;M4;M1;M65;M3;M38;M33;M42 |
| | QCGHYKHITSK | |
| 414 | ICYTPSKLIEYTDFA | M4;M49;M3;M48;M63;M41;M62;M4;M49;M3;M48;M63;M41;M62 |
| | TSACVLAAEC | |
| 415 | TPSKLIEYTDFATSA | M14;M29;M3;M48;M63;M41;M62;M14;M29;M3;M48;M63;M41;M62 |
| | CVLAAECTIF | |
| 416 | SAVGNICYTPSKLIE | M3;M48;M63;M41;M8;M43;M42;M3;M48;M63;M41;M8;M43;M42 |
| | YTDFATSACV | |
| 417 | PPIKDFGGFNFSQILP | M4;M32;M28;M14;M31;M1;M34;M29;M30;M33;M36;M27;M35;M22 |
| | DPSKPSKRS | |
| 418 | FSAVGNICYTPSKLI | M3;M48;M41;M8;M49;M43;M42;M3;M48;M41;M8;M49;M43;M42 |
| | EYTDFATSAC | |
| 419 | RVFSAVGNICYTPSK | M3;M48;M41;M8;M49;M43;M42;M3;M48;M41;M8;M49;M43;M42 |
| | LIEYTDFATS | |
| 420 | VFSAVGNICYTPSKL | M3;M48;M41;M8;M49;M43;M42;M3;M48;M41;M8;M49;M43;M42 |
| | IEYTDFATSA | |
| 421 | VGDYFVLTSHTVMP | M54;M16;M55;M60;M34;M29;M45;M51;M18;M42;M43;M15;M46; |
| | LSAPTLVPQEH | M39 |
| 422 | LAWPLIVTALRANS | M73;M60;M51;M46;M39;M11;M44;M73;M60;M51;M46;M39;M11; |
| | AVKLQNNELSP | M44 |
| 423 | VPANSTVLSFCAFA | M32;M12;M28;M48;M36;M8;M49;M32;M12;M28;M48;M36;M8;M49 |
| | VDAAKAYKDYL | |
| 424 | KSILSPLYAFASEAA | M21;M14;M6;M45;M48;M49;M22;M21;M14;M6;M45;M48;M49;M22 |
| | RVVRSIFSRT | |
| 425 | QKSILSPLYAFASEA | M21;M14;M6;M45;M48;M49;M22;M21;M14;M6;M45;M48;M49;M22 |
| | ARVVRSIFSR | |
| 426 | IPLTTAAKLMVVIPD | M28;M14;M24;M57;M51;M82;M23;M28;M14;M24;M57;M51;M82; |
| | YNTYKNTCDG | M23 |
| 427 | LSPLYAFASEAARV | M14;M6;M45;M48;M49;M22;M59;M14;M6;M45;M48;M49;M22;M59 |
| | VRSIFSRTLET | |
| 428 | ILSPLYAFASEAARV | M14;M6;M45;M48;M49;M22;M59;M14;M6;M45;M48;M49;M22;M59 |
| | VRSIFSRTLE | |
| 429 | KQFDTYNLWNTFTR | M71;M58;M14;M16;M54;M25;M34;M53;M60;M19;M17;M15;M42; |
| | LQSLENVAFNV | M39 |
| 430 | QFDTYNLWNTFTRL | M71;M58;M14;M16;M54;M25;M34;M53;M60;M19;M17;M15;M42; |
| | QSLENVAFNVV | M39 |
| 431 | FDTYNLWNTFTRLQ | M71;M58;M14;M16;M54;M25;M34;M53;M60;M19;M17;M15;M42; |
| | SLENVAFNVVN | M39 |
| 432 | LPDDFTGCVIAWNS | M13;M32;M56;M12;M14;M55;M29;M38;M48;M50;M67;M68;M49; |
| | NNLDSKVGGNY | M26 |
| 433 | NYKLPDDFTGCVIA | M13;M32;M56;M12;M14;M55;M29;M38;M48;M50;M67;M68;M49; |
| | WNSNNLDSKVG | M26 |
| 434 | KLPDDFTGCVIAWN | M13;M32;M56;M12;M14;M55;M29;M38;M48;M50;M67;M68;M49; |
| | SNNLDSKVGGN | M26 |
| 435 | YKLPDDFTGCVIAW | M13;M32;M56;M12;M14;M55;M29;M38;M48;M50;M67;M68;M49; |
| | NSNNLDSKVGG | M26 |
| 436 | GRCDIKDLPKEITVA | M64;M21;M12;M25;M6;M48;M33;M42;M43;M49;M46;M39;M22; |
| | TSRTLSYYKL | M23 |
| 437 | RGMVLGSLAATVRL | M16;M14;M55;M10;M53;M51;M17;M16;M14;M55;M10;M53;M51; |
| | QAGNATEVPAN | M17 |
| 438 | GMVLGSLAATVRL | M16;M14;M55;M10;M53;M51;M17;M16;M14;M55;M10;M53;M51; |
| | QAGNATEVPANS | M17 |
| 439 | TSNPTTFHLDGEVIT | M21;M7;M48;M45;M50;M36;M49;M21;M7;M48;M45;M50;M36;M49 |
| | FDNLKTLLSL | |
| 440 | YTSNPTTFHLDGEVI | M21;M7;M48;M45;M50;M36;M49;M21;M7;M48;M45;M50;M36;M49 |
| | TFDNLKTLLS | |
| 441 | STVLSFCAFAVDAA | M32;M12;M28;M48;M57;M36;M69;M32;M12;M28;M48;M57;M36; |
| | KAYKDYLASGG | M69 |
| 442 | FTLLLQLCTFTRSTN | M19;M14;M25;M33;M8;M42;M39;M19;M14;M25;M33;M8;M42; |
| | SRIKASMPTT | M39 |
| 443 | FFTLLLQLCTFTRST | M19;M14;M25;M33;M8;M42;M39;M19;M14;M25;M33;M8;M42; |
| | NSRIKASMPT | M39 |
| 444 | YITTYPGQGLNGYT | M75;M70;M45;M36;M63;M76;M39;M75;M70;M45;M36;M63;M76; |
| | VEEAKTVLKKC | M39 |
| 445 | AKKNNLPFKLTCAT | M21;M12;M19;M25;M2;M33;M8;M21;M12;M19;M25;M2;M33;M8 |
| | TRQVVNVVTTK | |
| 446 | KKNNLPFKLTCATT | M21;M12;M19;M25;M2;M33;M8;M21;M12;M19;M25;M2;M33;M8 |
| | RQVVNVVTTKI | |
| 447 | NGECPNFVFPLNSII | M28;M19;M29;M6;M61;M50;M72;M28;M19;M29;M6;M61;M50;M72 |
| | KTIQPRVEKK | |
| 448 | LNVGDYFVLTSHTV | M54;M16;M55;M25;M34;M29;M45;M18;M41;M42;M43;M15;M46; |
| | MPLSAPTLVPQ | M39 |
| 449 | YDTNVLEGSVAYES | M12;M14;M19;M34;M50;M18;M46;M12;M14;M19;M34;M50;M18; |
| | LRPDTRYVLMD | M46 |
| 450 | NVGDYFVLTSHTV | M54;M16;M55;M60;M34;M29;M45;M18;M41;M42;M43;M15;M46; |
| | MPLSAPTLVPQE | M39 |
| 451 | TLLSLREVRTIKVFT | M21;M14;M29;M6;M72;M26;M22;M21;M14;M29;M6;M72;M26;M22 |
| | TVDNINLHTQ | |
| 452 | SAAKKNNLPFKLTC | M21;M12;M19;M78;M25;M33;M8;M21;M12;M19;M78;M25;M33;M8 |
| | ATTRQVVNVVT | |
| 453 | AAKKNNLPFKLTCA | M21;M12;M19;M78;M25;M33;M8;M21;M12;M19;M78;M25;M33;M8 |
| | TTRQVVNVVTT | |
| 454 | MPYFFTLLLQLCTFT | M19;M25;M29;M33;M8;M42;M39;M19;M25;M29;M33;M8;M42;M39 |
| | RSTNSRIKAS | |
| 455 | NYMPYFFTLLLQLC | M19;M25;M29;M33;M8;M42;M39;M19;M25;M29;M33;M8;M42;M39 |
| | TFTRSTNSRIK | |
| 456 | YMPYFFTLLLQLCT | M19;M25;M29;M33;M8;M42;M39;M19;M25;M29;M33;M8;M42;M39 |
| | FTRSTNSRIKA | |
| 457 | EEIAIILASFSASTSA | M28;M14;M38;M33;M8;M43;M42;M28;M14;M38;M33;M8;M43;M42 |
| | FVETVKGLD | |
| 458 | LNEEIAIILASFSAST | M28;M14;M38;M33;M8;M43;M42;M28;M14;M38;M33;M8;M43;M42 |
| | SAFVETVKG | |
| 459 | EIAIILASFSASTSAF | M28;M14;M38;M33;M8;M43;M42;M28;M14;M38;M33;M8;M43;M42 |
| | VETVKGLDY | |
| 460 | NEEIAIILASFSASTS | M28;M14;M38;M33;M8;M43;M42;M28;M14;M38;M33;M8;M43;M42 |
| | AFVETVKGL | |
| 461 | GCVPLNIIPLTTAAK | M19;M14;M24;M38;M82;M57;M22;M19;M14;M24;M38;M82;M57; |
| | LMVVIPDYNT | M22 |
| 462 | LPSYAAFATAQEAY | M12;M28;M31;M70;M6;M33;M68;M12;M28;M31;M70;M6;M33;M68 |
| | EQAVANGDSEV | |
| 463 | DNVLSTFISAARQGF | M14;M70;M16;M29;M33;M18;M63;M14;M70;M16;M29;M33;M18; |
| | VDSDVETKDV | M63 |
| 464 | AKALRKVPTDNYIT | M16;M14;M29;M48;M49;M15;M26;M16;M14;M29;M48;M49;M15; |
| | TYPGQGLNGYT | M26 |
| 465 | FKLVNKFLALCADSI | M34;M45;M41;M67;M8;M42;M39;M34;M45;M41;M67;M8;M42;M39 |
| | IIGGAKLKAL | |
| 466 | TFFKLVNKFLALCA | M34;M45;M41;M67;M8;M42;M39;M34;M45;M41;M67;M8;M42;M39 |
| | DSIIIGGAKLK | |
| 467 | FFKLVNKFLALCAD | M34;M45;M41;M67;M8;M42;M39;M34;M45;M41;M67;M8;M42;M39 |
| | SIIIGGAKLKA | |
| 468 | DVVRQCSGVTFQSA | M71;M19;M1;M60;M25;M33;M26;M71;M19;M1;M60;M25;M33;M26 |
| | VKRTIKGTHHW | |
| 469 | CVMYASAVVLLILM | M75;M32;M28;M6;M72;M46;M39;M75;M32;M28;M6;M72;M46;M39 |
| | TARTVYDDGAR | |
| 470 | AAKAYKDYLASGG | M12;M37;M57;M33;M8;M42;M39;M12;M37;M57;M33;M8;M42;M39 |
| | QPITNCVKMLCT | |
| 471 | VLLPLTQYNRYLAL | M19;M14;M55;M53;M27;M42;M20;M19;M14;M55;M53;M27;M42; |
| | YNKYKYFSGAM | M20 |
| 472 | DVLLPLTQYNRYLA | M19;M14;M55;M53;M27;M42;M20;M19;M14;M55;M53;M27;M42; |
| | LYNKYKYFSGA | M20 |
| 473 | LLPLTQYNRYLALY | M19;M14;M55;M53;M27;M42;M20;M19;M14;M55;M53;M27;M42; |
| | NKYKYFSGAMD | M20 |
| 474 | LPLTQYNRYLALYN | M19;M14;M55;M53;M27;M42;M20;M19;M14;M55;M53;M27;M42; |
| | KYKYFSGAMDT | M20 |
| 475 | DNLKTLLSLREVRTI | M32;M14;M31;M29;M6;M49;M26;M32;M14;M31;M29;M6;M49;M26 |
| | KVFTTVDNIN | |
| 476 | GDFLHFLPRVFSAV | M64;M12;M24;M53;M45;M46;M22;M64;M12;M24;M53;M45;M46; |
| | GNICYTPSKLI | M22 |
| 477 | CPNFVFPLNSIIKTIQ | M28;M29;M6;M61;M72;M62;M59;M28;M29;M6;M61;M72;M62;M59 |
| | PRVEKKKLD | |
| 478 | VDTANPKTPKYKFV | M40;M77;M14;M34;M30;M29;M39;M40;M77;M14;M34;M30;M29; |
| | RIQPGQTFSVL | M39 |
| 479 | KVDTANPKTPKYKF | M40;M77;M14;M34;M30;M29;M39;M40;M77;M14;M34;M30;M29; |
| | VRIQPGQTFSV | M39 |
| 480 | VEQRKQDDKKIKAC | M28;M14;M65;M60;M74;M67;M49;M28;M14;M65;M60;M74;M67; |
| | VEEVTTTLEET | M49 |
| 481 | SVEQRKQDDKKIKA | M28;M14;M65;M60;M74;M67;M49;M28;M14;M65;M60;M74;M67; |
| | CVEEVTTTLEE | M49 |
| 482 | NVLEGSVAYESLRP | M19;M24;M7;M34;M50;M18;M46;M19;M24;M7;M34;M50;M18;M46 |
| | DTRYVLMDGSI | |
| 483 | FEYYHTTDPSFLGR | M66;M1;M25;M29;M6;M37;M33;M66;M1;M25;M29;M6;M37;M33 |
| | YMSALNHTKKW | |
| 484 | ASTSAFVETVKGLD | M54;M16;M1;M60;M6;M37;M52;M54;M16;M1;M60;M6;M37;M52 |
| | YKAFKQIVESC | |
| 485 | CDNIKFADDLNQLT | M64;M19;M70;M38;M63;M22;M23;M64;M19;M70;M38;M63;M22; |
| | GYKKPASRELK | M23 |
| 486 | ELAKNVSLDNVLST | M14;M7;M29;M61;M18;M27;M26;M14;M7;M29;M61;M18;M27; |
| | FISAARQGFVD | M26 |
| 487 | LTSSSKTPEEHFIETI | M64;M40;M16;M30;M29;M68;M8;M64;M40;M16;M30;M29;M68; |
| | SLAGSYKDW | M8 |
| 488 | CALAPNMMVTNNT | M11;M12;M24;M10;M6;M50;M9;M11;M12;M24;M10;M6;M50;M9 |
| | FTLKGGAPTKVT | |
| 489 | LAPNMMVTNNTFTL | M11;M12;M24;M10;M6;M50;M9;M11;M12;M24;M10;M6;M50;M9 |
| | KGGAPTKVTFG | |
| 490 | ALAPNMMVTNNTF | M11;M12;M24;M10;M6;M50;M9;M11;M12;M24;M10;M6;M50;M9 |
| | TLKGGAPTKVTF | |
| 491 | IKFADDLNQLTGYK | M58;M19;M70;M53;M38;M63;M15;M58;M19;M70;M53;M38;M63; |
| | KPASRELKVTF | M15 |
| 492 | FADDLNQLTGYKKP | M58;M19;M70;M53;M38;M63;M15;M58;M19;M70;M53;M38;M63; |
| | ASRELKVTFFP | M15 |
| 493 | NIKFADDLNQLTGY | M58;M19;M70;M53;M38;M63;M15;M58;M19;M70;M53;M38;M63; |
| | KKPASRELKVT | M15 |
| 494 | KFADDLNQLTGYK | M58;M19;M70;M53;M38;M63;M15;M58;M19;M70;M53;M38;M63; |
| | KPASRELKVTFF | M15 |
| 495 | PNYEDLLIRKSNHNF | M12;M24;M14;M70;M10;M9;M23;M12;M24;M14;M70;M10;M9; |
| | LVQAGNVQLR | M23 |
| 496 | NSPNLAWPLIVTAL | M73;M60;M18;M46;M39;M11;M44;M73;M60;M18;M46;M39;M11; |
| | RANSAVKLQNN | M44 |
| 497 | DNSPNLAWPLIVTA | M73;M60;M18;M46;M39;M11;M44;M73;M60;M18;M46;M39;M11; |
| | LRANSAVKLQN | M44 |
| 498 | QDDKKIKACVEEVT | M32;M28;M65;M6;M74;M67;M20;M32;M28;M65;M6;M74;M67;M20 |
| | TTLEETKFLTE | |
| 499 | DDKKIKACVEEVTT | M32;M28;M65;M6;M74;M67;M20;M32;M28;M65;M6;M74;M67;M20 |
| | TLEETKFLTEN | |
| 500 | SASIVAGGIVAIVVT | M21;M12;M60;M6;M62;M22;M59;M21;M12;M60;M6;M62;M22;M59 |
| | CLAYYFMRFR | |
| 501 | SIVAGGIVAIVVTCL | M21;M12;M60;M6;M62;M22;M59;M21;M12;M60;M6;M62;M22;M59 |
| | AYYFMRFRRA | |
| 502 | IVAGGIVAIVVTCLA | M21;M12;M60;M6;M62;M22;M59;M21;M12;M60;M6;M62;M22;M59 |
| | YYFMRFRRAF | |
| 503 | ASIVAGGIVAIVVTC | M21;M12;M60;M6;M62;M22;M59;M21;M12;M60;M6;M62;M22;M59 |
| | LAYYFMRFRR | |
| 504 | FACVVADAVIKTLQ | M28;M78;M45;M79;M49;M82;M47;M28;M78;M45;M79;M49;M82; |
| | PVSELLTPLGI | M47 |
| 505 | VVADAVIKTLQPVS | M28;M78;M45;M79;M49;M82;M47;M28;M78;M45;M79;M49;M82; |
| | ELLTPLGIDLD | M47 |
| 506 | ACVVADAVIKTLQP | M28;M78;M45;M79;M49;M82;M47;M28;M78;M45;M79;M49;M82; |
| | VSELLTPLGID | M47 |
| 507 | EFACVVADAVIKTL | M28;M78;M45;M79;M49;M82;M47;M28;M78;M45;M79;M49;M82; |
| | QPVSELLTPLG | M47 |
| 508 | CVVADAVIKTLQPV | M28;M78;M45;M79;M49;M82;M47;M28;M78;M45;M79;M49;M82; |
| | SELLTPLGIDL | M47 |
| 509 | LSGLDSLDTYPSLET | M21;M55;M34;M29;M48;M41;M22;M21;M55;M34;M29;M48;M41; |
| | IQITISSFKW | M22 |
| 510 | SGLDSLDTYPSLETI | M21;M55;M34;M29;M48;M41;M22;M21;M55;M34;M29;M48;M41; |
| | QITISSFKWD | M22 |
| 511 | CLSGLDSLDTYPSLE | M21;M55;M34;M29;M48;M41;M22;M21;M55;M34;M29;M48;M41; |
| | TIQITISSFK | M22 |
| 512 | KAPKEIIFLEGETLPT | M54;M24;M48;M42;M27;M46;M39;M54;M24;M48;M42;M27;M46; |
| | EVLTEEVVL | M39 |
| 513 | APKEIIFLEGETLPTE | M54;M24;M48;M42;M27;M46;M39;M54;M24;M48;M42;M27;M46; |
| | VLTEEVVLK | M39 |
| 514 | DPSFLGRYMSALNH | M19;M66;M24;M25;M74;M61;M33;M19;M66;M24;M25;M74;M61; |
| | TKKWKYPQVNG | M33 |
| 515 | ERSEKSYELQTPFEI | M12;M58;M24;M25;M34;M17;M39;M12;M58;M24;M25;M34;M17; |
| | KLAKKFDTFN | M39 |
| 516 | EKMADQAMTQMY | M56;M19;M55;M25;M74;M18;M82;M56;M19;M55;M25;M74;M18; |
| | KQARSEDKRAKVT | M82 |
| 517 | LEKMADQAMTQMY | M56;M19;M55;M25;M74;M18;M82;M56;M19;M55;M25;M74;M18; |
| | KQARSEDKRAKV | M82 |
| 518 | LAVFQSASKIITLKK | M56;M9;M58;M24;M16;M55;M60;M53;M74;M27;M17;M15;M52; |
| | RWQLALSKGV | M82 |
| 519 | VSSPDAVTAYNGYL | M24;M55;M48;M41;M49;M35;M26;M24;M55;M48;M41;M49;M35; |
| | TSSSKTPEEHF | M26 |
| 520 | SVSSPDAVTAYNGY | M24;M55;M48;M41;M49;M35;M26;M24;M55;M48;M41;M49;M35; |
| | LTSSSKTPEEH | M26 |
| 521 | VSVSSPDAVTAYNG | M24;M55;M48;M41;M49;M35;M26;M24;M55;M48;M41;M49;M35; |
| | YLTSSSKTPEE | M26 |
| 522 | VLNNDYYRSLPGVF | M25;M34;M45;M33;M42;M43;M39;M25;M34;M45;M33;M42;M43; |
| | CGVDAVNLLTN | M39 |
| 523 | VESDDYIATNGPLK | M11;M73;M10;M29;M18;M9;M44;M11;M73;M10;M29;M18;M9;M44 |
| | VGGSCVLSGHN | |
| 524 | QAVANGDSEVVLK | M71;M13;M55;M1;M53;M17;M59;M71;M13;M55;M1;M53;M17;M59 |
| | KLKKSLNVAKSE | |
| 525 | NASFDNFKFVCDNI | M40;M64;M21;M19;M25;M22;M23;M40;M64;M21;M19;M25;M22; |
| | KFADDLNQLTG | M23 |
| 526 | LHNDILLAKDTTEA | M21;M32;M58;M53;M36;M68;M22;M21;M32;M58;M53;M36;M68; |
| | FEKMVSLLSVL | M22 |
| 527 | CVQLHNDILLAKDT | M21;M32;M58;M53;M36;M68;M22;M21;M32;M58;M53;M36;M68; |
| | TEAFEKMVSLL | M22 |
| 528 | QLHNDILLAKDTTE | M21;M32;M58;M53;M36;M68;M22;M21;M32;M58;M53;M36;M68; |
| | AFEKMVSLLSV | M22 |
| 529 | VQLHNDILLAKDTT | M21;M32;M58;M53;M36;M68;M22;M21;M32;M58;M53;M36;M68; |
| | EAFEKMVSLLS | M22 |
| 530 | QCVQLHNDILLAKD | M21;M32;M58;M53;M36;M68;M22;M21;M32;M58;M53;M36;M68; |
| | TTEAFEKMVSL | M22 |
| 531 | GQQQTTLKGVEAV | M56;M54;M55;M60;M53;M6;M22;M56;M54;M55;M60;M53;M6;M22 |
| | MYMGTLSYEQFK | |
| 532 | VLLILMTARTVYDD | M75;M32;M6;M72;M46;M11;M59;M75;M32;M6;M72;M46;M11;M59 |
| | GARRVWTLMNV | |
| 533 | LILMTARTVYDDGA | M32;M5;M6;M72;M11;M59;M20;M32;M5;M6;M72;M11;M59;M20 |
| | RRVWTLMNVLT | |
| 534 | RATLQAIASEFSSLP | M21;M28;M41;M42;M43;M39;M22;M21;M28;M41;M42;M43;M39; |
| | SYAAFATAQE | M22 |
| 535 | FQHANLDSCKRVLN | M58;M16;M5;M2;M27;M17;M15;M58;M16;M5;M2;M27;M17;M15 |
| | VVCKTCGQQQT | |
| 536 | GDSCNNYMLTYNK | M53;M74;M8;M15;M52;M11;M47;M53;M74;M8;M15;M52;M11; |
| | VENMTPRDLGAC | M47 |
| 537 | LVFLFVAAIFYLITP | M56;M32;M1;M34;M18;M39;M47;M56;M32;M1;M34;M18;M39; |
| | VHVMSKHTDF | M47 |
| 538 | IDYDCVSFCYMHH | M24;M14;M18;M17;M43;M52;M9;M24;M14;M18;M17;M43;M52; |
| | MELPTGVHAGTD | M9 |
| 539 | FNIDYDCVSFCYMH | M24;M14;M18;M17;M43;M52;M9;M24;M14;M18;M17;M43;M52; |
| | HMELPTGVHAG | M9 |
| 540 | NIDYDCVSFCYMHH | M24;M14;M18;M17;M43;M52;M9;M24;M14;M18;M17;M43;M52; |
| | MELPTGVHAGT | M9 |
| 541 | GFNIDYDCVSFCYM | M24;M14;M18;M17;M43;M52;M9;M24;M14;M18;M17;M43;M52; |
| | HHMELPTGVHA | M9 |
| 542 | VGFNIDYDCVSFCY | M24;M14;M18;M17;M43;M52;M9;M24;M14;M18;M17;M43;M52; |
| | MHHMELPTGVH | M9 |
| 543 | SVGFNIDYDCVSFC | M24;M14;M18;M17;M43;M52;M9;M24;M14;M18;M17;M43;M52; |
| | YMHHMELPTGV | M9 |
| 544 | RYVLMDGSIIQFPNT | M48;M41;M43;M49;M42;M26;M22;M48;M41;M43;M49;M42;M26; |
| | YLEGSVRVVT | M22 |
| 545 | TRYVLMDGSIIQFPN | M48;M41;M43;M49;M42;M26;M22;M48;M41;M43;M49;M42;M26; |
| | TYLEGSVRVV | M22 |
| 546 | PDTRYVLMDGSIIQF | M48;M41;M43;M49;M42;M26;M22;M48;M41;M43;M49;M42;M26; |
| | PNTYLEGSVR | M22 |
| 547 | YVLMDGSIIQFPNTY | M48;M41;M43;M49;M42;M26;M22;M48;M41;M43;M49;M42;M26; |
| | LEGSVRVVTT | M22 |
| 548 | VLMDGSIIQFPNTYL | M48;M41;M43;M49;M42;M26;M22;M48;M41;M43;M49;M42;M26; |
| | EGSVRVVTTF | M22 |
| 549 | DTRYVLMDGSIIQFP | M48;M41;M43;M49;M42;M26;M22;M48;M41;M43;M49;M42;M26; |
| | NTYLEGSVRV | M22 |
| 550 | RKLEKMADQAMTQ | M56;M55;M25;M2;M74;M18;M82;M56;M55;M25;M2;M74;M18;M82 |
| | MYKQARSEDKRA | |
| 551 | DSEVVLKKLKKSLN | M32;M28;M55;M53;M74;M33;M68;M32;M28;M55;M53;M74;M33; |
| | VAKSEFDRDAA | M68 |
| 552 | TSVVLLSVLQQLRV | M40;M56;M11;M54;M34;M30;M22;M40;M56;M11;M54;M34;M30; |
| | ESSSKLWAQCV | M22 |
| 553 | IVGDVVQEGVLTAV | M16;M29;M38;M61;M67;M27;M17;M16;M29;M38;M61;M67;M27; |
| | VIPTKKAGGTT | M17 |
| 554 | IFSRTLETAQNSVRV | M54;M24;M5;M34;M60;M2;M9;M54;M24;M5;M34;M60;M2;M9 |
| | LQKAAITILD | |
| 555 | RTVYDDGARRVWT | M5;M2;M18;M52;M22;M47;M20;M5;M2;M18;M52;M22;M47;M20 |
| | LMNVLTLVYKVY | |
| 556 | TVYDDGARRVWTL | M5;M2;M18;M52;M22;M47;M20;M5;M2;M18;M52;M22;M47;M20 |
| | MNVLTLVYKVYY | |
| 557 | EGVLTAVVIPTKKA | M58;M16;M29;M53;M38;M67;M17;M58;M16;M29;M53;M38;M67; |
| | GGTTEMLAKAL | M17 |
| 558 | EGSVRVVTTFDSEY | M64;M48;M27;M20;M43;M22;M23;M64;M48;M27;M20;M43;M22; |
| | CRHGTCERSEA | M23 |
| 559 | GSVRVVTTFDSEYC | M64;M48;M27;M20;M43;M22;M23;M64;M48;M27;M20;M43;M22; |
| | RHGTCERSEAG | M23 |
| 560 | GSIIQFPNTYLEGSV | M64;M12;M20;M43;M42;M22;M23;M64;M12;M20;M43;M42;M22; |
| | RVVTTFDSEY | M23 |
| 561 | VRMYIFFASFYYVW | M21;M24;M1;M37;M35;M9;M23;M21;M24;M1;M37;M35;M9;M23 |
| | KSYVHVVDGCN | |
| 562 | RMYIFFASFYYVWK | M21;M24;M1;M37;M35;M9;M23;M21;M24;M1;M37;M35;M9;M23 |
| | SYVHVVDGCNS | |
| 563 | KALNLGETFVTHSK | M71;M24;M55;M50;M18;M27;M26;M71;M24;M55;M50;M18;M27; |
| | GLYRKCVKSRE | M26 |
| 564 | KLKALNLGETFVTH | M71;M24;M55;M50;M18;M27;M26;M71;M24;M55;M50;M18;M27; |
| | SKGLYRKCVKS | M26 |
| 565 | LNLGETFVTHSKGL | M71;M24;M55;M50;M18;M27;M26;M71;M24;M55;M50;M18;M27; |
| | YRKCVKSREET | M26 |
| 566 | ALNLGETFVTHSKG | M71;M24;M55;M50;M18;M27;M26;M71;M24;M55;M50;M18;M27; |
| | LYRKCVKSREE | M26 |
| 567 | LKALNLGETFVTHS | M71;M24;M55;M50;M18;M27;M26;M71;M24;M55;M50;M18;M27; |
| | KGLYRKCVKSR | M26 |
| 568 | LGETFVTHSKGLYR | M71;M24;M55;M50;M18;M52;M47;M71;M24;M55;M50;M18;M52; |
| | KCVKSREETGL | M47 |
| 569 | HEGKTFYVLPNDDT | M34;M74;M41;M42;M43;M39;M22;M34;M74;M41;M42;M43;M39; |
| | LRVEAFEYYHT | M22 |
| 570 | EGKTFYVLPNDDTL | M34;M74;M41;M42;M43;M39;M22;M34;M74;M41;M42;M43;M39; |
| | RVEAFEYYHTT | M22 |
| 571 | QTIEVNSFSGYLKLT | M56;M38;M50;M8;M9;M47;M23;M56;M38;M50;M8;M9;M47;M23 |
| | DNVYIKNADI | |
| 572 | AYIICISTKHFYWFF | M56;M32;M24;M10;M7;M50;M23;M56;M32;M24;M10;M7;M50; |
| | SNYLKRRVVF | M23 |
| 573 | NYAKPFLNKVVSTT | M78;M10;M37;M8;M15;M9;M23;M78;M10;M37;M8;M15;M9;M23 |
| | TNIVTRCLNRV | |
| 574 | ANYAKPFLNKVVST | M78;M10;M37;M8;M15;M9;M23;M78;M10;M37;M8;M15;M9;M23 |
| | TTNIVTRCLNR | |
| 575 | YAKPFLNKVVSTTT | M78;M10;M37;M8;M15;M9;M23;M78;M10;M37;M8;M15;M9;M23 |
| | NIVTRCLNRVC | |
| 576 | AKPFLNKVVSTTTNI | M78;M10;M37;M8;M15;M9;M23;M78;M10;M37;M8;M15;M9;M23 |
| | VTRCLNRVCT | |
| 577 | LVPFWITIAYIICIST | M56;M32;M24;M10;M7;M60;M23;M56;M32;M24;M10;M7;M60; |
| | KHFYWFFSN | M23 |
| 578 | PLVPFWITIAYIICIST | M56;M32;M24;M10;M7;M60;M23;M56;M32;M24;M10;M7;M60; |
| | KHFYWFFS | M23 |
| 579 | WITIAYIICISTKHFY | M56;M32;M24;M10;M7;M60;M23;M56;M32;M24;M10;M7;M60; |
| | WFFSNYLKR | M23 |
| 580 | TIAYIICISTKHFYWF | M56;M32;M24;M10;M7;M60;M23;M56;M32;M24;M10;M7;M60; |
| | FSNYLKRRV | M23 |
| 581 | ITIAYIICISTKHFYW | M56;M32;M24;M10;M7;M60;M23;M56;M32;M24;M10;M7;M60; |
| | FFSNYLKRR | M23 |
| 582 | FWITIAYIICISTKHF | M56;M32;M24;M10;M7;M60;M23;M56;M32;M24;M10;M7;M60; |
| | YWFFSNYLK | M23 |
| 583 | VPFWITIAYIICISTK | M56;M32;M24;M10;M7;M60;M23;M56;M32;M24;M10;M7;M60; |
| | HFYWFFSNY | M23 |
| 584 | PFWITIAYIICISTKH | M56;M32;M24;M10;M7;M60;M23;M56;M32;M24;M10;M7;M60; |
| | FYWFFSNYL | M23 |
| 585 | TPLVPFWITIAYIICIS | M56;M32;M24;M10;M7;M60;M23;M56;M32;M24;M10;M7;M60; |
| | TKHFYWFF | M23 |
| 586 | SHFVNLDNLRANNT | M64;M40;M31;M53;M43;M46;M39;M64;M40;M31;M53;M43;M46; |
| | KGSLPINVIVF | M39 |
| 587 | TTEAFEKMVSLLSV | M61;M74;M69;M43;M42;M39;M22;M61;M74;M69;M43;M42;M39; |
| | LLSMQGAVDIN | M22 |
| 588 | FNMVYMPASWVMR | M32;M52;M70;M83;M15;M46;M39;M32;M52;M70;M83;M15;M46; |
| | IMTWLDMVDTSL | M39 |
| 589 | LLEIKDTEKYCALAP | M78;M65;M25;M34;M18;M39;M82;M78;M65;M25;M34;M18;M39; |
| | NMMVTNNTFT | M82 |
| 590 | LEIKDTEKYCALAP | M78;M65;M25;M34;M18;M39;M82;M78;M65;M25;M34;M18;M39; |
| | NMMVTNNTFTL | M82 |
| 591 | DVKCTSVVLLSVLQ | M40;M56;M11;M54;M60;M34;M22;M40;M56;M11;M54;M60;M34; |
| | QLRVESSSKLW | M22 |
| 592 | KCTSVVLLSVLQQL | M40;M56;M11;M54;M60;M34;M22;M40;M56;M11;M54;M60;M34; |
| | RVESSSKLWAQ | M22 |
| 593 | VKCTSVVLLSVLQQ | M40;M56;M11;M54;M60;M34;M22;M40;M56;M11;M54;M60;M34; |
| | LRVESSSKLWA | M22 |
| 594 | CTSVVLLSVLQQLR | M40;M56;M11;M54;M60;M34;M22;M40;M56;M11;M54;M60;M34; |
| | VESSSKLWAQC | M22 |
| 595 | MSDVKCTSVVLLSV | M40;M56;M54;M60;M34;M29;M22;M40;M56;M54;M60;M34;M29; |
| | LQQLRVESSSK | M22 |
| 596 | PSFLGRYMSALNHT | M66;M24;M5;M25;M74;M61;M33;M66;M24;M5;M25;M74;M61; |
| | KKWKYPQVNGL | M33 |
| 597 | VGPNVNKGEDIQLL | M77;M32;M28;M31;M60;M38;M68;M77;M32;M28;M31;M60;M38; |
| | KSAYENFNQHE | M68 |
| 598 | VVGPNVNKGEDIQL | M77;M32;M28;M31;M60;M38;M68;M77;M32;M28;M31;M60;M38; |
| | LKSAYENFNQH | M68 |
| 599 | SAMQTMLFTMLRK | M16;M43;M35;M15;M52;M9;M47;M16;M43;M35;M15;M52;M9; |
| | LDNDALNNIINN | M47 |
| 600 | YTVEEAKTVLKKCK | M64;M54;M78;M65;M55;M60;M9;M64;M54;M78;M65;M55;M60; |
| | SAFYILPSIIS | M9 |
| 601 | GYTVEEAKTVLKKC | M64;M54;M78;M65;M55;M60;M9;M64;M54;M78;M65;M55;M60; |
| | KSAFYILPSII | M9 |
| 602 | YEKLKPVLDWLEEK | M32;M61;M57;M67;M27;M9;M20;M32;M61;M57;M67;M27;M9; |
| | FKEGVEFLRDG | M20 |
| 603 | EKLKPVLDWLEEKF | M32;M61;M57;M67;M27;M9;M20;M32;M61;M57;M67;M27;M9; |
| | KEGVEFLRDGW | M20 |
| 604 | LDQAISMWALIISVT | M64;M56;M32;M11;M68;M22;M23;M64;M56;M32;M11;M68;M22; |
| | SNYSGVVTTV | M23 |
| 605 | ALDQAISMWALIISV | M64;M56;M32;M11;M68;M22;M23;M64;M56;M32;M11;M68;M22; |
| | TSNYSGVVTT | M23 |
| 606 | YMPASWVMRIMTW | M64;M32;M70;M83;M15;M52;M22;M64;M32;M70;M83;M15;M52; |
| | LDMVDTSLSGFK | M22 |
| 607 | ITPVHVMSKHTDFSS | M56;M21;M53;M17;M52;M26;M47;M56;M21;M53;M17;M52;M26; |
| | EIIGYKAIDG | M47 |
| 608 | SCTLSEQLDFIDTKR | M64;M75;M66;M74;M83;M27;M22;M64;M75;M66;M74;M83;M27; |
| | GVYCCREHEH | M22 |
| 609 | TLSEQLDFIDTKRGV | M64;M75;M66;M74;M83;M27;M22;M64;M75;M66;M74;M83;M27; |
| | YCCREHEHEI | M22 |
| 610 | LSEQLDFIDTKRGV | M64;M75;M66;M74;M83;M27;M22;M64;M75;M66;M74;M83;M27; |
| | YCCREHEHEIA | M22 |
| 611 | CTLSEQLDFIDTKRG | M64;M75;M66;M74;M83;M27;M22;M64;M75;M66;M74;M83;M27; |
| | VYCCREHEHE | M22 |
| 612 | YLITPVHVMSKHTD | M56;M21;M53;M17;M35;M52;M47;M56;M21;M53;M17;M35;M52; |
| | FSSEIIGYKAI | M47 |
| 613 | IFYLITPVHVMSKHT | M56;M21;M53;M17;M35;M52;M47;M56;M21;M53;M17;M35;M52; |
| | DFSSEIIGYK | M47 |
| 614 | AIFYLITPVHVMSKH | M56;M21;M53;M17;M35;M52;M47;M56;M21;M53;M17;M35;M52; |
| | TDFSSEIIGY | M47 |
| 615 | FYLITPVHVMSKHT | M56;M21;M53;M17;M35;M52;M47;M56;M21;M53;M17;M35;M52; |
| | DFSSEIIGYKA | M47 |
| 616 | MLNPNYEDLLIRKS | M21;M24;M70;M10;M29;M9;M23;M21;M24;M70;M10;M29;M9; |
| | NHNFLVQAGNV | M23 |
| 617 | DMLNPNYEDLLIRK | M21;M24;M70;M10;M29;M9;M23;M21;M24;M70;M10;M29;M9; |
| | SNHNFLVQAGN | M23 |
| 618 | SRTLETAQNSVRVL | M56;M54;M24;M5;M34;M60;M9;M56;M54;M24;M5;M34;M60;M9 |
| | QKAAITILDGI | |
| 619 | FSRTLETAQNSVRV | M56;M54;M24;M5;M34;M60;M9;M56;M54;M24;M5;M34;M60;M9 |
| | LQKAAITILDG | |
| 620 | RTLETAQNSVRVLQ | M56;M54;M24;M5;M34;M60;M9;M56;M54;M24;M5;M34;M60;M9 |
| | KAAITILDGIS | |
| 621 | TEEVVLKTGDLQPL | M58;M54;M60;M38;M17;M62;M15;M58;M54;M60;M38;M17;M62; |
| | EQPTSEAVEAP | M15 |
| 622 | VLTEEVVLKTGDLQ | M58;M54;M60;M38;M17;M62;M15;M58;M54;M60;M38;M17;M62; |
| | PLEQPTSEAVE | M15 |
| 623 | LTEEVVLKTGDLQP | M58;M54;M60;M38;M17;M62;M15;M58;M54;M60;M38;M17;M62; |
| | LEQPTSEAVEA | M15 |
| 624 | EEVVLKTGDLQPLE | M58;M54;M60;M38;M17;M62;M15;M58;M54;M60;M38;M17;M62; |
| | QPTSEAVEAPL | M15 |
| 625 | KVKPTVVVNAANV | M56;M11;M73;M50;M18;M9;M23;M56;M11;M73;M50;M18;M9; |
| | YLKHGGGVAGAL | M23 |
| 626 | AKKVKPTVVVNAA | M56;M11;M73;M50;M18;M9;M23;M56;M11;M73;M50;M18;M9; |
| | NVYLKHGGGVAG | M23 |
| 627 | KKVKPTVVVNAAN | M56;M11;M73;M50;M18;M9;M23;M56;M11;M73;M50;M18;M9; |
| | VYLKHGGGVAGA | M23 |
| 628 | VKPTVVVNAANVY | M56;M11;M73;M50;M18;M9;M23;M56;M11;M73;M50;M18;M9; |
| | LKHGGGVAGALN | M23 |
| 629 | AMVRMYIFFASFYY | M21;M24;M37;M20;M35;M9;M23;M21;M24;M37;M20;M35;M9; |
| | VWKSYVHVVDG | M23 |
| 630 | AVLDMCASLKELLQ | M56;M24;M10;M60;M50;M18;M59;M56;M24;M10;M60;M50;M18; |
| | NGMNGRTILGS | M59 |
| 631 | VVQTIEVNSFSGYL | M56;M38;M50;M26;M9;M47;M23;M56;M38;M50;M26;M9;M47; |
| | KLTDNVYIKNA | M23 |
| 632 | PVVQTIEVNSFSGYL | M56;M38;M50;M26;M9;M47;M23;M56;M38;M50;M26;M9;M47; |
| | KLTDNVYIKN | M23 |
| 633 | NTTKGGRFVLALLS | M77;M32;M70;M31;M33;M68;M20;M77;M32;M70;M31;M33;M68; |
| | DLQDLKWARFP | M20 |
| 634 | YNTTKGGRFVLALL | M77;M32;M70;M31;M33;M68;M20;M77;M32;M70;M31;M33;M68; |
| | SDLQDLKWARF | M20 |
| 635 | YYNTTKGGRFVLAL | M77;M32;M70;M31;M33;M68;M20;M77;M32;M70;M31;M33;M68; |
| | LSDLQDLKWAR | M20 |
| 636 | TPPIKDFGGFNFSQIL | M4;M32;M28;M14;M31;M1;M34;M29;M30;M3;M33;M36;M27;M35; |
| | PDPSKPSKR | M22 |
| 637 | SASKIITLKKRWQLA | M9;M58;M24;M55;M60;M53;M74;M33;M42;M27;M8;M17;M15; |
| | LSKGVHFVCN | M52;M82 |
| 638 | QSASKIITLKKRWQL | M9;M58;M24;M55;M60;M53;M74;M33;M42;M27;M8;M17;M15; |
| | ALSKGVHFVC | M52;M82 |
| 639 | EAVMYMGTLSYEQ | M4;M40;M16;M1;M3;M6;M37;M52;M4;M40;M16;M1;M3;M6;M37; |
| | FKKGVQIPCTCG | M52 |
| 640 | VEAVMYMGTLSYE | M4;M40;M16;M1;M3;M6;M37;M52;M4;M40;M16;M1;M3;M6;M37; |
| | QFKKGVQIPCTC | M52 |
| 641 | TFTCASEYTGNYQC | M4;M1;M38;M3;M41;M8;M43;M42;M4;M1;M38;M3;M41;M8;M43; |
| | GHYKHITSKET | M42 |
| 642 | YTPSKLIEYTDFATS | M4;M14;M29;M3;M48;M63;M41;M62;M4;M14;M29;M3;M48;M63; |
| | ACVLAAECTI | M41;M62 |
| 643 | VGNICYTPSKLIEYT | M4;M3;M48;M63;M41;M8;M49;M42;M4;M3;M48;M63;M41;M8; |
| | DFATSACVLA | M49;M42 |
| 644 | GNICYTPSKLIEYTD | M4;M3;M48;M63;M41;M8;M49;M42;M4;M3;M48;M63;M41;M8; |
| | FATSACVLAA | M49;M42 |
| 645 | GTFTCASEYTGNYQ | M4;M1;M65;M3;M38;M33;M43;M42;M4;M1;M65;M3;M38;M33; |
| | CGHYKHITSKE | M43;M42 |
| 646 | FVETVKGLDYKAFK | M4;M54;M1;M3;M37;M42;M52;M47;M4;M54;M1;M3;M37;M42; |
| | QIVESCGNFKV | M52;M47 |
| 647 | AFVETVKGLDYKAF | M4;M54;M1;M3;M37;M42;M52;M47;M4;M54;M1;M3;M37;M42; |
| | KQIVESCGNFK | M52;M47 |
| 648 | VETVKGLDYKAFK | M4;M54;M1;M3;M37;M42;M52;M47;M4;M54;M1;M3;M37;M42; |
| | QIVESCGNFKVT | M52;M47 |
| 649 | LTSQWLTNIFGTVY | M4;M28;M31;M34;M3;M45;M67;M59;M4;M28;M31;M34;M3;M45; |
| | EKLKPVLDWLE | M67;M59 |
| 650 | AVGNICYTPSKLIEY | M3;M48;M63;M41;M8;M49;M43;M42;M3;M48;M63;M41;M8;M49; |
| | TDFATSACVL | M43;M42 |
| 651 | LNNLNRGMVLGSL | M12;M16;M14;M55;M10;M53;M51;M17;M12;M16;M14;M55;M10; |
| | AATVRLQAGNAT | M53;M51;M17 |
| 652 | LNRGMVLGSLAAT | M12;M16;M14;M55;M10;M53;M51;M17;M12;M16;M14;M55;M10; |
| | VRLQAGNATEVP | M53;M51;M17 |
| 653 | NLNRGMVLGSLAA | M12;M16;M14;M55;M10;M53;M51;M17;M12;M16;M14;M55;M10; |
| | TVRLQAGNATEV | M53;M51;M17 |
| 654 | NRGMVLGSLAATV | M12;M16;M14;M55;M10;M53;M51;M17;M12;M16;M14;M55;M10; |
| | RLQAGNATEVPA | M53;M51;M17 |
| 655 | NNLNRGMVLGSLA | M12;M16;M14;M55;M10;M53;M51;M17;M12;M16;M14;M55;M10; |
| | ATVRLQAGNATE | M53;M51;M17 |
| 656 | RCDIKDLPKEITVAT | M64;M56;M21;M12;M55;M25;M6;M48;M33;M42;M43;M49;M46; |
| | SRTLSYYKLG | M39;M22;M23 |
| 657 | KLKTLVATAEAELA | M12;M81;M28;M29;M6;M45;M38;M49;M12;M81;M28;M29;M6; |
| | KNVSLDNVLST | M45;M38;M49 |
| 658 | EKLKTLVATAEAEL | M12;M81;M28;M29;M6;M45;M38;M49;M12;M81;M28;M29;M6; |
| | AKNVSLDNVLS | M45;M38;M49 |
| 659 | EFSSLPSYAAFATAQ | M12;M14;M28;M70;M31;M6;M68;M52;M12;M14;M28;M70;M31; |
| | EAYEQAVANG | M6;M68;M52 |
| 660 | FSSLPSYAAFATAQE | M12;M14;M28;M70;M31;M6;M68;M52;M12;M14;M28;M70;M31; |
| | AYEQAVANGD | M6;M68;M52 |
| 661 | NIIPLTTAAKLMVVI | M28;M14;M24;M38;M57;M51;M82;M23;M28;M14;M24;M38;M57; |
| | PDYNTYKNTC | M51;M82;M23 |
| 662 | LNIIPLTTAAKLMVV | M28;M14;M24;M38;M57;M51;M82;M23;M28;M14;M24;M38;M57; |
| | IPDYNTYKNT | M51;M82;M23 |
| 663 | IIPLTTAAKLMVVIP | M28;M14;M24;M38;M57;M51;M82;M23;M28;M14;M24;M38;M57; |
| | DYNTYKNTCD | M51;M82;M23 |
| 664 | PLNIIPLTTAAKLMV | M28;M14;M24;M38;M57;M51;M82;M23;M28;M14;M24;M38;M57; |
| | VIPDYNTYKN | M51;M82;M23 |
| 665 | NPTTFHLDGEVITFD | M21;M30;M45;M48;M50;M36;M49;M22;M21;M30;M45;M48;M50; |
| | NLKTLLSLRE | M36;M49;M22 |
| 666 | SNPTTFHLDGEVITF | M21;M30;M45;M48;M50;M36;M49;M22;M21;M30;M45;M48;M50; |
| | DNLKTLLSLR | M36;M49;M22 |
| 667 | ITTYPGQGLNGYTV | M75;M70;M25;M45;M36;M63;M76;M39;M75;M70;M25;M45;M36; |
| | EEAKTVLKKCK | M63;M76;M39 |
| 668 | TTYPGQGLNGYTVE | M75;M70;M25;M45;M36;M63;M76;M39;M75;M70;M25;M45;M36; |
| | EAKTVLKKCKS | M63;M76;M39 |
| 669 | LREVRTIKVFTTVD | M21;M29;M6;M51;M43;M42;M26;M22;M21;M29;M6;M51;M43; |
| | NINLHTQVVDM | M42;M26;M22 |
| 670 | FDVVRQCSGVTFQS | M4;M71;M19;M1;M60;M25;M33;M26;M4;M71;M19;M1;M60;M25; |
| | AVKRTIKGTHH | M33;M26 |
| 671 | PFDVVRQCSGVTFQ | M4;M71;M19;M1;M60;M25;M33;M26;M4;M71;M19;M1;M60;M25; |
| | SAVKRTIKGTH | M33;M26 |
| 672 | KQDDKKIKACVEEV | M32;M28;M14;M65;M6;M74;M67;M49;M32;M28;M14;M65;M6; |
| | TTTLEETKFLT | M74;M67;M49 |
| 673 | KHITSKETLYCIDGA | M10;M36;M43;M35;M46;M39;M9;M44;M10;M36;M43;M35;M46; |
| | LLTKSSEYKG | M39;M9;M44 |
| 674 | HITSKETLYCIDGAL | M10;M36;M43;M35;M46;M39;M9;M44;M10;M36;M43;M35;M46; |
| | LTKSSEYKGP | M39;M9;M44 |
| 675 | YKHITSKETLYCIDG | M10;M36;M43;M35;M46;M39;M9;M44;M10;M36;M43;M35;M46; |
| | ALLTKSSEYK | M39;M9;M44 |
| 676 | ITSKETLYCIDGALL | M10;M36;M43;M35;M46;M39;M9;M44;M10;M36;M43;M35;M46; |
| | TKSSEYKGPI | M39;M9;M44 |
| 677 | VYCPRHVICTSEDM | M64;M28;M6;M45;M72;M49;M59;M20;M64;M28;M6;M45;M72; |
| | LNPNYEDLLIR | M49;M59;M20 |
| 678 | CPRHVICTSEDMLN | M64;M28;M6;M45;M72;M49;M59;M20;M64;M28;M6;M45;M72; |
| | PNYEDLLIRKS | M49;M59;M20 |
| 679 | YCPRHVICTSEDML | M64;M28;M6;M45;M72;M49;M59;M20;M64;M28;M6;M45;M72; |
| | NPNYEDLLIRK | M49;M59;M20 |
| 680 | KTLLSLREVRTIKVF | M21;M14;M29;M6;M72;M49;M26;M22;M21;M14;M29;M6;M72; |
| | TTVDNINLHT | M49;M26;M22 |
| 681 | IWNVKDFMSLSEQL | M56;M19;M66;M25;M6;M45;M18;M59;M56;M19;M66;M25;M6; |
| | RKQIRSAAKKN | M45;M18;M59 |
| 682 | NVKDFMSLSEQLRK | M56;M19;M66;M25;M6;M45;M18;M59;M56;M19;M66;M25;M6; |
| | QIRSAAKKNNL | M45;M18;M59 |
| 683 | WNVKDFMSLSEQL | M56;M19;M66;M25;M6;M45;M18;M59;M56;M19;M66;M25;M6; |
| | RKQIRSAAKKNN | M45;M18;M59 |
| 684 | HFLPRVFSAVGNICY | M64;M12;M45;M42;M8;M43;M46;M22;M64;M12;M45;M42;M8; |
| | TPSKLIEYTD | M43;M46;M22 |
| 685 | FLHFLPRVFSAVGNI | M64;M12;M45;M42;M8;M43;M46;M22;M64;M12;M45;M42;M8; |
| | CYTPSKLIEY | M43;M46;M22 |
| 686 | LHFLPRVFSAVGNIC | M64;M12;M45;M42;M8;M43;M46;M22;M64;M12;M45;M42;M8; |
| | YTPSKLIEYT | M43;M46;M22 |
| 687 | LLSLREVRTIKVFTT | M21;M14;M29;M6;M72;M42;M26;M22;M21;M14;M29;M6;M72; |
| | VDNINLHTQV | M42;M26;M22 |
| 688 | FLPRVFSAVGNICYT | M64;M12;M45;M8;M49;M43;M42;M22;M64;M12;M45;M8;M49; |
| | PSKLIEYTDF | M43;M42;M22 |
| 689 | LPRVFSAVGNICYTP | M64;M12;M45;M8;M49;M43;M42;M22;M64;M12;M45;M8;M49; |
| | SKLIEYTDFA | M43;M42;M22 |
| 690 | QVESDDYIATNGPL | M11;M73;M19;M10;M29;M18;M9;M44;M11;M73;M19;M10;M29; |
| | KVGGSCVLSGH | M18;M9;M44 |
| 691 | NAMQVESDDYIATN | M11;M73;M19;M10;M29;M18;M9;M44;M11;M73;M19;M10;M29; |
| | GPLKVGGSCVL | M18;M9;M44 |
| 692 | NNAMQVESDDYIAT | M11;M73;M19;M10;M29;M18;M9;M44;M11;M73;M19;M10;M29; |
| | NGPLKVGGSCV | M18;M9;M44 |
| 693 | AMQVESDDYIATNG | M11;M73;M19;M10;M29;M18;M9;M44;M11;M73;M19;M10;M29; |
| | PLKVGGSCVLS | M18;M9;M44 |
| 694 | MQVESDDYIATNGP | M11;M73;M19;M10;M29;M18;M9;M44;M11;M73;M19;M10;M29; |
| | LKVGGSCVLSG | M18;M9;M44 |
| 695 | QVAKSHNIALIWNV | M32;M28;M66;M1;M6;M37;M72;M68;M32;M28;M66;M1;M6;M37; |
| | KDFMSLSEQLR | M72;M68 |
| 696 | DILLAKDTTEAFEK | M40;M16;M34;M30;M53;M36;M42;M39;M40;M16;M34;M30;M53; |
| | MVSLLSVLLSM | M36;M42;M39 |
| 697 | SLPSYAAFATAQEA | M12;M28;M31;M70;M6;M33;M68;M52;M12;M28;M31;M70;M6; |
| | YEQAVANGDSE | M33;M68;M52 |
| 698 | CGHYKHITSKETLY | M21;M58;M36;M41;M8;M43;M39;M9;M21;M58;M36;M41;M8;M43; |
| | CIDGALLTKSS | M39;M9 |
| 699 | DNYITTYPGQGLNG | M75;M70;M26;M29;M45;M63;M76;M39;M75;M70;M26;M29;M45; |
| | YTVEEAKTVLK | M63;M76;M39 |
| 700 | RKVPTDNYITTYPG | M16;M14;M29;M48;M41;M49;M15;M26;M16;M14;M29;M48;M41; |
| | QGLNGYTVEEA | M49;M15;M26 |
| 701 | ALRKVPTDNYITTY | M16;M14;M29;M48;M41;M49;M15;M26;M16;M14;M29;M48;M41; |
| | PGQGLNGYTVE | M49;M15;M26 |
| 702 | LRKVPTDNYITTYP | M16;M14;M29;M48;M41;M49;M15;M26;M16;M14;M29;M48;M41; |
| | GQGLNGYTVEE | M49;M15;M26 |
| 703 | KALRKVPTDNYITT | M16;M14;M29;M48;M41;M49;M15;M26;M16;M14;M29;M48;M41; |
| | YPGQGLNGYTV | M49;M15;M26 |
| 704 | SLDNVLSTFISAARQ | M14;M70;M16;M29;M61;M33;M18;M63;M14;M70;M16;M29;M61; |
| | GFVDSDVETK | M33;M18;M63 |
| 705 | LDNVLSTFISAARQG | M14;M70;M16;M29;M61;M33;M18;M63;M14;M70;M16;M29;M61; |
| | FVDSDVETKD | M33;M18;M63 |
| 706 | TPEEHFIETISLAGSY | M64;M40;M32;M14;M16;M29;M6;M68;M64;M40;M32;M14;M16; |
| | KDWSYSGQS | M29;M6;M68 |
| 707 | GQGLNGYTVEEAKT | M75;M19;M60;M25;M45;M76;M39;M9;M75;M19;M60;M25;M45; |
| | VLKKCKSAFYI | M76;M39;M9 |
| 708 | QGLNGYTVEEAKTV | M75;M19;M78;M60;M45;M76;M39;M9;M75;M19;M78;M60;M45; |
| | LKKCKSAFYIL | M76;M39;M9 |
| 709 | YASAVVLLILMTAR | M75;M32;M28;M6;M72;M46;M39;M59;M75;M32;M28;M6;M72; |
| | TVYDDGARRVW | M46;M39;M59 |
| 710 | VMYASAVVLLILMT | M75;M32;M28;M6;M72;M46;M39;M59;M75;M32;M28;M6;M72; |
| | ARTVYDDGARR | M46;M39;M59 |
| 711 | MYASAVVLLILMTA | M75;M32;M28;M6;M72;M46;M39;M59;M75;M32;M28;M6;M72; |
| | RTVYDDGARRV | M46;M39;M59 |
| 712 | VVSTTTNIVTRCLNR | M4;M64;M78;M1;M6;M37;M69;M20;M4;M64;M78;M1;M6;M37; |
| | VCTNYMPYFF | M69;M20 |
| 713 | KVVSTTTNIVTRCL | M4;M64;M78;M1;M6;M37;M69;M20;M4;M64;M78;M1;M6;M37; |
| | NRVCTNYMPYF | M69;M20 |
| 714 | VSTTTNIVTRCLNRV | M4;M64;M78;M1;M6;M37;M69;M20;M4;M64;M78;M1;M6;M37; |
| | CTNYMPYFFT | M69;M20 |
| 715 | TDPSFLGRYMSALN | M19;M66;M24;M1;M25;M61;M37;M33;M19;M66;M24;M1;M25; |
| | HTKKWKYPQVN | M61;M37;M33 |
| 716 | TTDPSFLGRYMSAL | M19;M66;M24;M1;M25;M61;M37;M33;M19;M66;M24;M1;M25; |
| | NHTKKWKYPQV | M61;M37;M33 |
| 717 | HTTDPSFLGRYMSA | M19;M66;M24;M1;M25;M61;M37;M33;M19;M66;M24;M1;M25; |
| | LNHTKKWKYPQ | M61;M37;M33 |
| 718 | AYKDYLASGGQPIT | M56;M12;M24;M53;M37;M33;M8;M39;M56;M12;M24;M53;M37; |
| | NCVKMLCTHTG | M33;M8;M39 |
| 719 | LTLGVYDYLVSTQE | M40;M31;M34;M30;M6;M8;M43;M42;M40;M31;M34;M30;M6;M8; |
| | FRYMNSQGLLP | M43;M42 |
| 720 | KLEKMADQAMTQM | M56;M19;M55;M25;M2;M74;M18;M82;M56;M19;M55;M25;M2; |
| | YKQARSEDKRAK | M74;M18;M82 |
| 721 | ECPNFVFPLNSIIKTI | M28;M29;M6;M61;M50;M72;M62;M59;M28;M29;M6;M61;M50; |
| | QPRVEKKKL | M72;M62;M59 |
| 722 | NEFACVVADAVIKT | M28;M78;M45;M79;M49;M39;M82;M47;M28;M78;M45;M79;M49; |
| | LQPVSELLTPL | M39;M82;M47 |
| 723 | KFVCDNIKFADDLN | M40;M64;M21;M19;M70;M63;M22;M23;M40;M64;M21;M19;M70; |
| | QLTGYKKPASR | M63;M22;M23 |
| 724 | VCDNIKFADDLNQL | M40;M64;M21;M19;M70;M63;M22;M23;M40;M64;M21;M19;M70; |
| | TGYKKPASREL | M63;M22;M23 |
| 725 | FVCDNIKFADDLNQ | M40;M64;M21;M19;M70;M63;M22;M23;M40;M64;M21;M19;M70; |
| | LTGYKKPASRE | M63;M22;M23 |
| 726 | EQRKQDDKKIKACV | M32;M28;M14;M65;M60;M74;M67;M49;M32;M28;M14;M65;M60; |
| | EEVTTTLEETK | M74;M67;M49 |
| 727 | QRKQDDKKIKACVE | M32;M28;M14;M65;M60;M74;M67;M49;M32;M28;M14;M65;M60; |
| | EVTTTLEETKF | M74;M67;M49 |
| 728 | TDNYITTYPGQGLN | M75;M70;M26;M29;M48;M63;M49;M76;M75;M70;M26;M29;M48; |
| | GYTVEEAKTVL | M63;M49;M76 |
| 729 | MDGSIIQFPNTYLEG | M12;M48;M41;M43;M49;M42;M26;M22;M12;M48;M41;M43;M49; |
| | SVRVVTTFDS | M42;M26;M22 |
| 730 | LMDGSIIQFPNTYLE | M12;M48;M41;M43;M49;M42;M26;M22;M12;M48;M41;M43;M49; |
| | GSVRVVTTFD | M42;M26;M22 |
| 731 | DNIKFADDLNQLTG | M64;M58;M19;M70;M53;M38;M63;M15;M64;M58;M19;M70;M53; |
| | YKKPASRELKV | M38;M63;M15 |
| 732 | VVLLILMTARTVYD | M75;M32;M6;M72;M46;M39;M11;M59;M75;M32;M6;M72;M46; |
| | DGARRVWTLMN | M39;M11;M59 |
| 733 | LNGYTVEEAKTVLK | M54;M78;M65;M55;M60;M45;M39;M9;M54;M78;M65;M55;M60; |
| | KCKSAFYILPS | M45;M39;M9 |
| 734 | GHYKHITSKETLYCI | M21;M58;M10;M46;M36;M43;M39;M9;M21;M58;M10;M46;M36; |
| | DGALLTKSSE | M43;M39;M9 |
| 735 | KNVSLDNVLSTFISA | M14;M16;M29;M61;M33;M18;M27;M26;M14;M16;M29;M61;M33; |
| | ARQGFVDSDV | M18;M27;M26 |
| 736 | VSLDNVLSTFISAAR | M14;M16;M29;M61;M33;M18;M27;M26;M14;M16;M29;M61;M33; |
| | QGFVDSDVET | M18;M27;M26 |
| 737 | AKNVSLDNVLSTFIS | M14;M16;M29;M61;M33;M18;M27;M26;M14;M16;M29;M61;M33; |
| | AARQGFVDSD | M18;M27;M26 |
| 738 | NVSLDNVLSTFISAA | M14;M16;M29;M61;M33;M18;M27;M26;M14;M16;M29;M61;M33; |
| | RQGFVDSDVE | M18;M27;M26 |
| 739 | FTPLVPFWITIAYIICI | M56;M32;M24;M10;M7;M60;M8;M23;M56;M32;M24;M10;M7;M60; |
| | STKHFYWF | M8;M23 |
| 740 | MFTPLVPFWITIAYII | M56;M32;M24;M10;M7;M60;M8;M23;M56;M32;M24;M10;M7;M60; |
| | CISTKHFYW | M8;M23 |
| 741 | MPLKAPKEIIFLEGE | M54;M24;M60;M48;M42;M27;M46;M39;M54;M24;M60;M48;M42; |
| | TLPTEVLTEE | M27;M46;M39 |
| 742 | LMPLKAPKEIIFLEG | M54;M24;M60;M48;M42;M27;M46;M39;M54;M24;M60;M48;M42; |
| | ETLPTEVLTE | M27;M46;M39 |
| 743 | LKAPKEIIFLEGETLP | M54;M24;M60;M48;M42;M27;M46;M39;M54;M24;M60;M48;M42; |
| | TEVLTEEVV | M27;M46;M39 |
| 744 | PLKAPKEIIFLEGETL | M54;M24;M60;M48;M42;M27;M46;M39;M54;M24;M60;M48;M42; |
| | PTEVLTEEV | M27;M46;M39 |
| 745 | LLMPLKAPKEIIFLE | M54;M24;M60;M48;M42;M27;M46;M39;M54;M24;M60;M48;M42; |
| | GETLPTEVLT | M27;M46;M39 |
| 746 | NKVVSTTTNIVTRC | M4;M64;M23;M78;M1;M37;M69;M20;M4;M64;M23;M78;M1;M37; |
| | LNRVCTNYMPY | M69;M20 |
| 747 | SHEGKTFYVLPNDD | M34;M74;M63;M41;M42;M43;M39;M22;M34;M74;M63;M41;M42; |
| | TLRVEAFEYYH | M43;M39;M22 |
| 748 | NSHEGKTFYVLPND | M34;M74;M63;M41;M42;M43;M39;M22;M34;M74;M63;M41;M42; |
| | DTLRVEAFEYY | M43;M39;M22 |
| 749 | VAYESLRPDTRYVL | M25;M34;M48;M50;M41;M43;M42;M39;M25;M34;M48;M50;M41; |
| | MDGSIIQFPNT | M43;M42;M39 |
| 750 | ATLQAIASEFSSLPS | M21;M28;M31;M41;M42;M43;M39;M22;M21;M28;M31;M41;M42; |
| | YAAFATAQEA | M43;M39;M22 |
| 751 | NLDSCKRVLNVVCK | M58;M16;M5;M2;M27;M17;M15;M52;M58;M16;M5;M2;M27;M17; |
| | TCGQQQTTLKG | M15;M52 |
| 752 | QHANLDSCKRVLN | M58;M16;M5;M2;M27;M17;M15;M52;M58;M16;M5;M2;M27;M17; |
| | VVCKTCGQQQTT | M15;M52 |
| 753 | LDSCKRVLNVVCKT | M58;M16;M5;M2;M27;M17;M15;M52;M58;M16;M5;M2;M27;M17; |
| | CGQQQTTLKGV | M15;M52 |
| 754 | ANLDSCKRVLNVVC | M58;M16;M5;M2;M27;M17;M15;M52;M58;M16;M5;M2;M27;M17; |
| | KTCGQQQTTLK | M15;M52 |
| 755 | HANLDSCKRVLNV | M58;M16;M5;M2;M27;M17;M15;M52;M58;M16;M5;M2;M27;M17; |
| | VCKTCGQQQTTL | M15;M52 |
| 756 | LNPNYEDLLIRKSN | M21;M14;M70;M24;M10;M29;M9;M23;M21;M14;M70;M24;M10; |
| | HNFLVQAGNVQ | M29;M9;M23 |
| 757 | CAKEIKESVQTFFKL | M16;M24;M53;M17;M43;M42;M39;M47;M16;M24;M53;M17;M43; |
| | VNKFLALCAD | M42;M39;M47 |
| 758 | DTEKYCALAPNMM | M11;M9;M24;M78;M10;M34;M6;M82;M11;M9;M24;M78;M10;M34; |
| | VTNNTFTLKGGA | M6;M82 |
| 759 | LTPVVQTIEVNSFSG | M56;M60;M29;M38;M6;M50;M26;M47;M56;M60;M29;M38;M6; |
| | YLKLTDNVYI | M50;M26;M47 |
| 760 | ELTPVVQTIEVNSFS | M56;M60;M29;M38;M6;M50;M26;M47;M56;M60;M29;M38;M6; |
| | GYLKLTDNVY | M50;M26;M47 |
| 761 | VYDDGARRVWTLM | M24;M5;M2;M18;M52;M22;M47;M20;M24;M5;M2;M18;M52;M22; |
| | NVLTLVYKVYYG | M47;M20 |
| 762 | YDDGARRVWTLMN | M24;M5;M2;M18;M52;M22;M47;M20;M24;M5;M2;M18;M52;M22; |
| | VLTLVYKVYYGN | M47;M20 |
| 763 | DDGARRVWTLMNV | M24;M5;M2;M18;M52;M22;M47;M20;M24;M5;M2;M18;M52;M22; |
| | LTLVYKVYYGNA | M47;M20 |
| 764 | PNTYLEGSVRVVTT | M64;M40;M48;M27;M20;M43;M22;M23;M64;M40;M48;M27;M20; |
| | FDSEYCRHGTC | M43;M22;M23 |
| 765 | TYLEGSVRVVTTFD | M64;M40;M48;M27;M20;M43;M22;M23;M64;M40;M48;M27;M20; |
| | SEYCRHGTCER | M43;M22;M23 |
| 766 | LEGSVRVVTTFDSE | M64;M40;M48;M27;M20;M43;M22;M23;M64;M40;M48;M27;M20; |
| | YCRHGTCERSE | M43;M22;M23 |
| 767 | NTYLEGSVRVVTTF | M64;M40;M48;M27;M20;M43;M22;M23;M64;M40;M48;M27;M20; |
| | DSEYCRHGTCE | M43;M22;M23 |
| 768 | YLEGSVRVVTTFDS | M64;M40;M48;M27;M20;M43;M22;M23;M64;M40;M48;M27;M20; |
| | EYCRHGTCERS | M43;M22;M23 |
| 769 | DYLASGGQPITNCV | M4;M64;M56;M24;M10;M53;M22;M23;M4;M64;M56;M24;M10; |
| | KMLCTHTGTGQ | M53;M22;M23 |
| 770 | VYLAVFDKNLYDK | M56;M70;M55;M41;M42;M49;M43;M39;M56;M70;M55;M41;M42; |
| | LVSSFLEMKSEK | M49;M43;M39 |
| 771 | IIQFPNTYLEGSVRV | M64;M40;M12;M27;M20;M43;M22;M23;M64;M40;M12;M27;M20; |
| | VTTFDSEYCR | M43;M22;M23 |
| 772 | QEGVLTAVVIPTKK | M58;M16;M29;M53;M38;M61;M67;M17;M58;M16;M29;M53;M38; |
| | AGGTTEMLAKA | M61;M67;M17 |
| 773 | VQEGVLTAVVIPTK | M58;M16;M29;M53;M38;M61;M67;M17;M58;M16;M29;M53;M38; |
| | KAGGTTEMLAK | M61;M67;M17 |
| 774 | VVQEGVLTAVVIPT | M58;M16;M29;M53;M38;M61;M67;M17;M58;M16;M29;M53;M38; |
| | KKAGGTTEMLA | M61;M67;M17 |
| 775 | SGGQPITNCVKMLC | M4;M64;M21;M56;M58;M53;M22;M23;M4;M64;M21;M56;M58; |
| | THTGTGQAITV | M53;M22;M23 |
| 776 | EMELTPVVQTIEVN | M56;M60;M29;M6;M38;M50;M47;M59;M56;M60;M29;M6;M38; |
| | SFSGYLKLTDN | M50;M47;M59 |
| 777 | NGYTVEEAKTVLKK | M64;M54;M78;M65;M55;M60;M39;M9;M64;M54;M78;M65;M55; |
| | CKSAFYILPSI | M60;M39;M9 |
| 778 | FFASFYYVWKSYVH | M21;M24;M1;M34;M37;M35;M9;M23;M21;M24;M1;M34;M37;M35; |
| | VVDGCNSSTCM | M9;M23 |
| 779 | FASFYYVWKSYVH | M21;M24;M1;M34;M37;M35;M9;M23;M21;M24;M1;M34;M37;M35; |
| | VVDGCNSSTCMM | M9;M23 |
| 780 | MYIFFASFYYVWKS | M21;M24;M1;M34;M37;M35;M9;M23;M21;M24;M1;M34;M37;M35; |
| | YVHVVDGCNSS | M9;M23 |
| 781 | IFFASFYYVWKSYV | M21;M24;M1;M34;M37;M35;M9;M23;M21;M24;M1;M34;M37;M35; |
| | HVVDGCNSSTC | M9;M23 |
| 782 | YIFFASFYYVWKSY | M21;M24;M1;M34;M37;M35;M9;M23;M21;M24;M1;M34;M37;M35; |
| | VHVVDGCNSST | M9;M23 |
| 783 | TVEEAKTVLKKCKS | M64;M54;M16;M78;M65;M55;M60;M9;M64;M54;M16;M78;M65; |
| | AFYILPSIISN | M55;M60;M9 |
| 784 | MVRMYIFFASFYYV | M21;M24;M1;M37;M20;M35;M9;M23;M21;M24;M1;M37;M20;M35; |
| | WKSYVHVVDGC | M9;M23 |
| 785 | IAYIICISTKHFYWFF | M56;M32;M24;M10;M7;M60;M50;M23;M56;M32;M24;M10;M7;M60; |
| | SNYLKRRVV | M50;M23 |
| 786 | DTTEAFEKMVSLLS | M53;M61;M74;M69;M42;M43;M39;M22;M53;M61;M74;M69;M42; |
| | VLLSMQGAVDI | M43;M39;M22 |
| 787 | TSAMQTMLFTMLR | M16;M17;M35;M15;M52;M43;M9;M47;M16;M17;M35;M15;M52; |
| | KLDNDALNNIIN | M43;M9;M47 |
| 788 | KSVGKFCLEASFNY | M54;M25;M60;M18;M41;M43;M42;M39;M54;M25;M60;M18;M41; |
| | LKSPNFSKLIN | M43;M42;M39 |
| 789 | VKSVGKFCLEASFN | M54;M25;M60;M18;M41;M43;M42;M39;M54;M25;M60;M18;M41; |
| | YLKSPNFSKLI | M43;M42;M39 |
| 790 | TVKSVGKFCLEASF | M54;M25;M60;M18;M41;M43;M42;M39;M54;M25;M60;M18;M41; |
| | NYLKSPNFSKL | M43;M42;M39 |
| 791 | NTVKSVGKFCLEAS | M54;M25;M60;M18;M41;M43;M42;M39;M54;M25;M60;M18;M41; |
| | FNYLKSPNFSK | M43;M42;M39 |
| 792 | LMLLEIKDTEKYCA | M9;M78;M65;M25;M34;M18;M39;M82;M9;M78;M65;M25;M34; |
| | LAPNMMVTNNT | M18;M39;M82 |
| 793 | MLLEIKDTEKYCAL | M9;M78;M65;M25;M34;M18;M39;M82;M9;M78;M65;M25;M34; |
| | APNMMVTNNTF | M18;M39;M82 |
| 794 | LITPVHVMSKHTDF | M56;M21;M53;M17;M35;M52;M26;M47;M56;M21;M53;M17;M35; |
| | SSEIIGYKAID | M52;M26;M47 |
| 795 | DQAISMWALIISVTS | M64;M56;M32;M11;M38;M68;M22;M23;M64;M56;M32;M11;M38; |
| | NYSGVVTTVM | M68;M22;M23 |
| 796 | AISMWALIISVTSNY | M64;M56;M32;M11;M38;M68;M22;M23;M64;M56;M32;M11;M38; |
| | SGVVTTVMFL | M68;M22;M23 |
| 797 | SMWALIISVTSNYSG | M64;M56;M32;M11;M38;M68;M22;M23;M64;M56;M32;M11;M38; |
| | VVTTVMFLAR | M68;M22;M23 |
| 798 | ISMWALIISVTSNYS | M64;M56;M32;M11;M38;M68;M22;M23;M64;M56;M32;M11;M38; |
| | GVVTTVMFLA | M68;M22;M23 |
| 799 | QAISMWALIISVTSN | M64;M56;M32;M11;M38;M68;M22;M23;M64;M56;M32;M11;M38; |
| | YSGVVTTVMF | M68;M22;M23 |
| 800 | TPVHVMSKHTDFSS | M56;M21;M53;M41;M17;M52;M26;M47;M56;M21;M53;M41;M17; |
| | EIIGYKAIDGG | M52;M26;M47 |
| 801 | TKGGRFVLALLSDL | M77;M32;M70;M31;M60;M33;M68;M20;M77;M32;M70;M31;M60; |
| | QDLKWARFPKS | M33;M68;M20 |
| 802 | TTKGGRFVLALLSD | M77;M32;M70;M31;M60;M33;M68;M20;M77;M32;M70;M31;M60; |
| | LQDLKWARFPK | M33;M68;M20 |
| 803 | PKCDRAMPNMLRI | M56;M21;M12;M54;M25;M30;M48;M69;M41;M42;M8;M49;M43; |
| | MASLVLARKHTT | M46;M39;M22;M23 |
| 804 | YPKCDRAMPNMLRI | M56;M21;M12;M54;M25;M30;M48;M69;M41;M42;M8;M49;M43; |
| | MASLVLARKHT | M46;M39;M22;M23 |
| 805 | DIKDLPKEITVATSR | M64;M56;M21;M12;M55;M25;M29;M6;M48;M33;M42;M43;M49; |
| | TLSYYKLGAS | M46;M39;M22;M23 |
| 806 | IKDLPKEITVATSRT | M64;M56;M21;M12;M55;M25;M29;M6;M48;M33;M42;M43;M49; |
| | LSYYKLGASQ | M46;M39;M22;M23 |
| 807 | CDIKDLPKEITVATS | M64;M56;M21;M12;M55;M25;M29;M6;M48;M33;M42;M43;M49; |
| | RTLSYYKLGA | M46;M39;M22;M23 |
| 808 | AVFQSASKIITLKKR | M56;M9;M58;M24;M16;M55;M60;M53;M74;M33;M42;M27;M8; |
| | WQLALSKGVH | M17;M15;M52;M82 |
| 809 | FQSASKIITLKKRWQ | M56;M9;M58;M24;M16;M55;M60;M53;M74;M33;M42;M27;M8; |
| | LALSKGVHFV | M17;M15;M52;M82 |
| 810 | VFQSASKIITLKKRW | M56;M9;M58;M24;M16;M55;M60;M53;M74;M33;M42;M27;M8; |
| | QLALSKGVHF | M17;M15;M52;M82 |
| 811 | CYTPSKLIEYTDFAT | M4;M49;M14;M29;M3;M48;M63;M41;M62;M4;M49;M14;M29;M3; |
| | SACVLAAECT | M48;M63;M41;M62 |
| 812 | SAFVETVKGLDYKA | M4;M54;M1;M3;M6;M37;M42;M52;M47;M4;M54;M1;M3;M6;M37; |
| | FKQIVESCGNF | M42;M52;M47 |
| 813 | RAMPNMLRIMASLV | M56;M21;M12;M54;M25;M30;M48;M69;M41;M42;M8;M49;M43; |
| | LARKHTTCCSL | M46;M39;M22;M35;M23 |
| 814 | CDRAMPNMLRIMA | M56;M21;M12;M54;M25;M30;M48;M69;M41;M42;M8;M49;M43; |
| | SLVLARKHTTCC | M46;M39;M22;M35;M23 |
| 815 | DRAMPNMLRIMASL | M56;M21;M12;M54;M25;M30;M48;M69;M41;M42;M8;M49;M43; |
| | VLARKHTTCCS | M46;M39;M22;M35;M23 |
| 816 | KCDRAMPNMLRIM | M56;M21;M12;M54;M25;M30;M48;M69;M41;M42;M8;M49;M43; |
| | ASLVLARKHTTC | M46;M39;M22;M35;M23 |
| 817 | YPGQGLNGYTVEEA | M75;M19;M70;M25;M45;M36;M63;M76;M39;M75;M19;M70;M25; |
| | KTVLKKCKSAF | M45;M36;M63;M76;M39 |
| 818 | TYPGQGLNGYTVEE | M75;M19;M70;M25;M45;M36;M63;M76;M39;M75;M19;M70;M25; |
| | AKTVLKKCKSA | M45;M36;M63;M76;M39 |
| 819 | KDLPKEITVATSRTL | M64;M56;M21;M12;M70;M55;M25;M29;M6;M48;M33;M42;M43; |
| | SYYKLGASQR | M49;M46;M39;M22;M23 |
| 820 | EVGFVVPGLPGTILR | M64;M11;M12;M24;M1;M6;M45;M8;M9;M64;M11;M12;M24;M1; |
| | TTNGDFLHFL | M6;M45;M8;M9 |
| 821 | WEIVKFISTCACEIV | M12;M29;M6;M45;M74;M72;M8;M22;M20;M12;M29;M6;M45;M74; |
| | GGQIVTCAKE | M72;M8;M22;M20 |
| 822 | TVLSFCAFAVDAAK | M32;M12;M28;M70;M48;M57;M36;M69;M63;M32;M12;M28;M70; |
| | AYKDYLASGGQ | M48;M57;M36;M69;M63 |
| 823 | SLREVRTIKVFTTVD | M21;M14;M29;M6;M51;M43;M42;M26;M22;M21;M14;M29;M6; |
| | NINLHTQVVD | M51;M43;M42;M26;M22 |
| 824 | STFNVPMEKLKTLV | M12;M81;M28;M29;M6;M45;M38;M49;M22;M12;M81;M28;M29; |
| | ATAEAELAKNV | M6;M45;M38;M49;M22 |
| 825 | SSTFNVPMEKLKTL | M12;M81;M28;M29;M6;M45;M38;M49;M22;M12;M81;M28;M29; |
| | VATAEAELAKN | M6;M45;M38;M49;M22 |
| 826 | SEFSSLPSYAAFATA | M12;M14;M28;M70;M31;M6;M68;M52;M22;M12;M14;M28;M70; |
| | QEAYEQAVAN | M31;M6;M68;M52;M22 |
| 827 | ASEFSSLPSYAAFAT | M12;M14;M28;M70;M31;M6;M68;M52;M22;M12;M14;M28;M70; |
| | AQEAYEQAVA | M31;M6;M68;M52;M22 |
| 828 | VPLNIIPLTTAAKLM | M28;M14;M22;M24;M38;M57;M51;M82;M23;M28;M14;M22;M24; |
| | VVIPDYNTYK | M38;M57;M51;M82;M23 |
| 829 | LKTLVATAEAELAK | M73;M12;M81;M28;M29;M6;M45;M38;M49;M73;M12;M81;M28; |
| | NVSLDNVLSTF | M29;M6;M45;M38;M49 |
| 830 | PMEKLKTLVATAEA | M12;M81;M28;M29;M6;M45;M38;M49;M59;M12;M81;M28;M29; |
| | ELAKNVSLDNV | M6;M45;M38;M49;M59 |
| 831 | NVPMEKLKTLVATA | M12;M81;M28;M29;M6;M45;M38;M49;M59;M12;M81;M28;M29; |
| | EAELAKNVSLD | M6;M45;M38;M49;M59 |
| 832 | MEKLKTLVATAEAE | M12;M81;M28;M29;M6;M45;M38;M49;M59;M12;M81;M28;M29; |
| | LAKNVSLDNVL | M6;M45;M38;M49;M59 |
| 833 | VPMEKLKTLVATAE | M12;M81;M28;M29;M6;M45;M38;M49;M59;M12;M81;M28;M29; |
| | AELAKNVSLDN | M6;M45;M38;M49;M59 |
| 834 | RAKVTSAMQTMLF | M16;M3;M17;M35;M15;M52;M43;M9;M47;M16;M3;M17;M35;M15; |
| | TMLRKLDNDALN | M52;M43;M9;M47 |
| 835 | AKVTSAMQTMLFT | M16;M3;M17;M35;M15;M52;M43;M9;M47;M16;M3;M17;M35;M15; |
| | MLRKLDNDALNN | M52;M43;M9;M47 |
| 836 | KVTSAMQTMLFTM | M16;M3;M17;M35;M15;M52;M43;M9;M47;M16;M3;M17;M35;M15; |
| | LRKLDNDALNNI | M52;M43;M9;M47 |
| 837 | VTSAMQTMLFTML | M16;M3;M17;M35;M15;M52;M43;M9;M47;M16;M3;M17;M35;M15; |
| | RKLDNDALNNII | M52;M43;M9;M47 |
| 838 | SSLPSYAAFATAQE | M12;M14;M28;M70;M31;M6;M33;M68;M52;M12;M14;M28;M70; |
| | AYEQAVANGDS | M31;M6;M33;M68;M52 |
| 839 | LIWNVKDFMSLSEQ | M56;M28;M66;M19;M25;M6;M45;M18;M59;M56;M28;M66;M19; |
| | LRKQIRSAAKK | M25;M6;M45;M18;M59 |
| 840 | VGFVVPGLPGTILRT | M64;M11;M24;M1;M6;M45;M8;M9;M23;M64;M11;M24;M1;M6; |
| | TNGDFLHFLP | M45;M8;M9;M23 |
| 841 | TPFDVVRQCSGVTF | M4;M71;M54;M19;M1;M60;M25;M46;M26;M4;M71;M54;M19;M1; |
| | QSAVKRTIKGT | M60;M25;M46;M26 |
| 842 | GECPNFVFPLNSIIKT | M28;M19;M29;M6;M61;M50;M72;M62;M59;M28;M19;M29;M6; |
| | IQPRVEKKK | M61;M50;M72;M62;M59 |
| 843 | DAAKAYKDYLASG | M12;M48;M37;M57;M33;M69;M8;M42;M39;M12;M48;M37;M57; |
| | GQPITNCVKMLC | M33;M69;M8;M42;M39 |
| 844 | VVYCPRHVICTSED | M64;M28;M66;M6;M45;M72;M49;M59;M20;M64;M28;M66;M6; |
| | MLNPNYEDLLI | M45;M72;M49;M59;M20 |
| 845 | DVVYCPRHVICTSE | M64;M28;M66;M6;M45;M72;M49;M59;M20;M64;M28;M66;M6; |
| | DMLNPNYEDLL | M45;M72;M49;M59;M20 |
| 846 | RKQDDKKIKACVEE | M32;M28;M14;M65;M60;M6;M74;M67;M49;M32;M28;M14;M65; |
| | VTTTLEETKFL | M60;M6;M74;M67;M49 |
| 847 | DFLHFLPRVFSAVG | M64;M12;M53;M45;M42;M8;M43;M46;M22;M64;M12;M53;M45; |
| | NICYTPSKLIE | M42;M8;M43;M46;M22 |
| 848 | LSLREVRTIKVFTTV | M21;M14;M29;M6;M72;M43;M42;M26;M22;M21;M14;M29;M6; |
| | DNINLHTQVV | M72;M43;M42;M26;M22 |
| 849 | QTFFKLVNKFLALC | M24;M34;M45;M41;M67;M42;M8;M43;M39;M24;M34;M45;M41; |
| | ADSIIIGGAKL | M67;M42;M8;M43;M39 |
| 850 | RPDTRYVLMDGSIIQ | M7;M30;M48;M41;M43;M49;M42;M26;M22;M7;M30;M48;M41; |
| | FPNTYLEGSV | M43;M49;M42;M26;M22 |
| 851 | ASAVVLLILMTART | M75;M32;M28;M6;M72;M46;M39;M11;M59;M75;M32;M28;M6; |
| | VYDDGARRVWT | M72;M46;M39;M11;M59 |
| 852 | SAVVLLILMTARTV | M75;M32;M28;M6;M72;M46;M39;M11;M59;M75;M32;M28;M6; |
| | YDDGARRVWTL | M72;M46;M39;M11;M59 |
| 853 | AVVLLILMTARTVY | M75;M32;M28;M6;M72;M46;M39;M11;M59;M75;M32;M28;M6; |
| | DDGARRVWTLM | M72;M46;M39;M11;M59 |
| 854 | LAKNVSLDNVLSTFI | M14;M16;M7;M29;M61;M33;M18;M27;M26;M14;M16;M7;M29; |
| | SAARQGFVDS | M61;M33;M18;M27;M26 |
| 855 | FDNLKTLLSLREVR | M32;M14;M66;M31;M65;M29;M6;M68;M49;M32;M14;M66;M31; |
| | TIKVFTTVDNI | M65;M29;M6;M68;M49 |
| 856 | TFDNLKTLLSLREV | M32;M14;M66;M31;M65;M29;M6;M68;M49;M32;M14;M66;M31; |
| | RTIKVFTTVDN | M65;M29;M6;M68;M49 |
| 857 | NPNYEDLLIRKSNH | M21;M12;M14;M70;M24;M10;M29;M9;M23;M21;M12;M14;M70; |
| | NFLVQAGNVQL | M24;M10;M29;M9;M23 |
| 858 | GLPGTILRTTNGDFL | M64;M75;M11;M24;M53;M6;M45;M52;M9;M64;M75;M11;M24; |
| | HFLPRVFSAV | M53;M6;M45;M52;M9 |
| 859 | ETSNSFDVLKSEDA | M64;M34;M30;M6;M41;M42;M46;M39;M22;M64;M34;M30;M6; |
| | QGMDNLACEDL | M41;M42;M46;M39;M22 |
| 860 | GEVITFDNLKTLLSL | M32;M66;M31;M65;M30;M29;M36;M68;M49;M32;M66;M31;M65; |
| | REVRTIKVFT | M30;M29;M36;M68;M49 |
| 861 | PTDNYITTYPGQGL | M75;M70;M26;M29;M48;M63;M49;M15;M76;M75;M70;M26;M29; |
| | NGYTVEEAKTV | M48;M63;M49;M15;M76 |
| 862 | SVGKFCLEASFNYL | M54;M19;M25;M60;M18;M41;M43;M42;M39;M54;M19;M25;M60; |
| | KSPNFSKLINI | M18;M41;M43;M42;M39 |
| 863 | VGKFCLEASFNYLK | M54;M19;M25;M60;M18;M41;M43;M42;M39;M54;M19;M25;M60; |
| | SPNFSKLINII | M18;M41;M43;M42;M39 |
| 864 | IQFPNTYLEGSVRVV | M64;M40;M12;M48;M27;M20;M43;M22;M23;M64;M40;M12;M48; |
| | TTFDSEYCRH | M27;M20;M43;M22;M23 |
| 865 | QFPNTYLEGSVRVV | M64;M40;M12;M48;M27;M20;M43;M22;M23;M64;M40;M12;M48; |
| | TTFDSEYCRHG | M27;M20;M43;M22;M23 |
| 866 | FPNTYLEGSVRVVT | M64;M40;M12;M48;M27;M20;M43;M22;M23;M64;M40;M12;M48; |
| | TFDSEYCRHGT | M27;M20;M43;M22;M23 |
| 867 | TGLLMPLKAPKEIIF | M54;M24;M31;M60;M48;M42;M27;M46;M39;M54;M24;M31;M60; |
| | LEGETLPTEV | M48;M42;M27;M46;M39 |
| 868 | GLLMPLKAPKEIIFL | M54;M24;M31;M60;M48;M42;M27;M46;M39;M54;M24;M31;M60; |
| | EGETLPTEVL | M48;M42;M27;M46;M39 |
| 869 | EVITFDNLKTLLSLR | M32;M66;M31;M65;M29;M30;M6;M68;M49;M32;M66;M31;M65; |
| | EVRTIKVFTT | M29;M30;M6;M68;M49 |
| 870 | VITFDNLKTLLSLRE | M32;M66;M31;M65;M29;M30;M6;M68;M49;M32;M66;M31;M65; |
| | VRTIKVFTTV | M29;M30;M6;M68;M49 |
| 871 | ITFDNLKTLLSLREV | M32;M66;M31;M65;M29;M30;M6;M68;M49;M32;M66;M31;M65; |
| | RTIKVFTTVD | M29;M30;M6;M68;M49 |
| 872 | HNSHEGKTFYVLPN | M31;M34;M74;M63;M41;M42;M43;M39;M22;M31;M34;M74;M63; |
| | DDTLRVEAFEY | M41;M42;M43;M39;M22 |
| 873 | RSEKSYELQTPFEIK | M12;M58;M24;M10;M25;M34;M17;M39;M23;M12;M58;M24;M10; |
| | LAKKFDTFNG | M25;M34;M17;M39;M23 |
| 874 | GDSEVVLKKLKKSL | M71;M32;M28;M55;M53;M74;M33;M68;M17;M71;M32;M28;M55; |
| | NVAKSEFDRDA | M53;M74;M33;M68;M17 |
| 875 | LSHFVNLDNLRANN | M64;M40;M31;M30;M53;M43;M46;M39;M22;M64;M40;M31;M30; |
| | TKGSLPINVIV | M53;M43;M46;M39;M22 |
| 876 | EIKDTEKYCALAPN | M78;M65;M25;M34;M6;M82;M18;M39;M9;M78;M65;M25;M34; |
| | MMVTNNTFTLK | M6;M82;M18;M39;M9 |
| 877 | IKDTEKYCALAPNM | M78;M65;M25;M34;M6;M82;M18;M39;M9;M78;M65;M25;M34;M6; |
| | MVTNNTFTLKG | M82;M18;M39;M9 |
| 878 | GGQIVTCAKEIKESV | M56;M55;M65;M10;M53;M79;M33;M8;M23;M56;M55;M65;M10; |
| | QTFFKLVNKF | M53;M79;M33;M8;M23 |
| 879 | KAGQKTYERHSLSH | M64;M40;M70;M31;M30;M15;M43;M39;M22;M64;M40;M70;M31; |
| | FVNLDNLRANN | M30;M15;M43;M39;M22 |
| 880 | TLQAIASEFSSLPSY | M21;M28;M31;M41;M42;M68;M43;M39;M22;M21;M28;M31;M41; |
| | AAFATAQEAY | M42;M68;M43;M39;M22 |
| 881 | NIFGTVYEKLKPVL | M32;M28;M31;M34;M67;M68;M39;M47;M20;M32;M28;M31;M34; |
| | DWLEEKFKEGV | M67;M68;M39;M47;M20 |
| 882 | FGTVYEKLKPVLDW | M32;M28;M31;M34;M67;M68;M39;M9;M20;M32;M28;M31;M34; |
| | LEEKFKEGVEF | M67;M68;M39;M9;M20 |
| 883 | KDTEKYCALAPNM | M11;M9;M24;M78;M65;M10;M34;M6;M82;M11;M9;M24;M78;M65; |
| | MVTNNTFTLKGG | M10;M34;M6;M82 |
| 884 | ELQTPFEIKLAKKFD | M23;M12;M58;M24;M10;M57;M27;M17;M20;M23;M12;M58;M24; |
| | TFNGECPNFV | M10;M57;M27;M17;M20 |
| 885 | SYELQTPFEIKLAKK | M23;M12;M58;M24;M10;M57;M27;M17;M20;M23;M12;M58;M24; |
| | FDTFNGECPN | M10;M57;M27;M17;M20 |
| 886 | YELQTPFEIKLAKKF | M23;M12;M58;M24;M10;M57;M27;M17;M20;M23;M12;M58;M24; |
| | DTFNGECPNF | M10;M57;M27;M17;M20 |
| 887 | LQTPFEIKLAKKFDT | M23;M12;M58;M24;M10;M57;M27;M17;M20;M23;M12;M58;M24; |
| | FNGECPNFVF | M10;M57;M27;M17;M20 |
| 888 | MELTPVVQTIEVNSF | M56;M60;M29;M6;M38;M50;M26;M47;M59;M56;M60;M29;M6; |
| | SGYLKLTDNV | M38;M50;M26;M47;M59 |
| 889 | VGDVVQEGVLTAV | M58;M16;M29;M53;M38;M61;M67;M27;M17;M58;M16;M29;M53; |
| | VIPTKKAGGTTE | M38;M61;M67;M27;M17 |
| 890 | DVVQEGVLTAVVIP | M58;M16;M29;M53;M38;M61;M67;M27;M17;M58;M16;M29;M53; |
| | TKKAGGTTEML | M38;M61;M67;M27;M17 |
| 891 | GDVVQEGVLTAVVI | M58;M16;M29;M53;M38;M61;M67;M27;M17;M58;M16;M29;M53; |
| | PTKKAGGTTEM | M38;M61;M67;M27;M17 |
| 892 | ASGGQPITNCVKML | M4;M64;M21;M56;M58;M10;M53;M22;M23;M4;M64;M21;M56; |
| | CTHTGTGQAIT | M58;M10;M53;M22;M23 |
| 893 | NLGETFVTHSKGLY | M71;M24;M55;M50;M18;M27;M52;M26;M47;M71;M24;M55;M50; |
| | RKCVKSREETG | M18;M27;M52;M26;M47 |
| 894 | AVFDKNLYDKLVSS | M31;M70;M25;M34;M41;M42;M49;M43;M39;M31;M70;M25;M34; |
| | FLEMKSEKQVE | M41;M42;M49;M43;M39 |
| 895 | ASFNYLKSPNFSKLI | M54;M25;M60;M74;M18;M41;M43;M42;M39;M54;M25;M60;M74; |
| | NIIIWFLLLS | M18;M41;M43;M42;M39 |
| 896 | VFLFVAAIFYLITPV | M56;M32;M34;M53;M18;M17;M52;M39;M47;M56;M32;M34;M53; |
| | HVMSKHTDFS | M18;M17;M52;M39;M47 |
| 897 | FLFVAAIFYLITPVH | M56;M32;M34;M53;M18;M17;M52;M39;M47;M56;M32;M34;M53; |
| | VMSKHTDFSS | M18;M17;M52;M39;M47 |
| 898 | FTCASEYTGNYQCG | M4;M52;M1;M38;M3;M41;M42;M8;M43;M39;M4;M52;M1;M38; |
| | HYKHITSKETL | M3;M41;M42;M8;M43;M39 |
| 899 | STSAFVETVKGLDY | M4;M54;M16;M1;M60;M3;M6;M37;M52;M47;M4;M54;M16;M1; |
| | KAFKQIVESCG | M60;M3;M6;M37;M52;M47 |
| 900 | TSAFVETVKGLDYK | M4;M54;M1;M60;M3;M6;M37;M42;M52;M47;M4;M54;M1;M60; |
| | AFKQIVESCGN | M3;M6;M37;M42;M52;M47 |
| 901 | TSQWLTNIFGTVYE | M4;M28;M31;M34;M3;M45;M47;M67;M39;M59;M4;M28;M31;M34; |
| | KLKPVLDWLEE | M3;M45;M47;M67;M39;M59 |
| 902 | GWEIVKFISTCACEI | M12;M29;M6;M45;M74;M72;M8;M49;M22;M20;M12;M29;M6;M45; |
| | VGGQIVTCAK | M74;M72;M8;M49;M22;M20 |
| 903 | TNIFGTVYEKLKPVL | M32;M28;M31;M34;M3;M67;M68;M39;M47;M20;M32;M28;M31; |
| | DWLEEKFKEG | M34;M3;M67;M68;M39;M47;M20 |
| 904 | LSFCAFAVDAAKAY | M32;M21;M12;M28;M70;M48;M57;M36;M69;M63;M32;M21;M12; |
| | KDYLASGGQPI | M28;M70;M48;M57;M36;M69;M63 |
| 905 | VLSFCAFAVDAAKA | M32;M21;M12;M28;M70;M48;M57;M36;M69;M63;M32;M21;M12; |
| | YKDYLASGGQP | M28;M70;M48;M57;M36;M69;M63 |
| 906 | IVKFISTCACEIVGG | M28;M66;M12;M78;M65;M29;M6;M74;M45;M79;M28;M66;M12; |
| | QIVTCAKEIK | M78;M65;M29;M6;M74;M45;M79 |
| 907 | FNVPMEKLKTLVAT | M12;M81;M28;M29;M6;M45;M38;M49;M22;M59;M12;M81;M28; |
| | AEAELAKNVSL | M29;M6;M45;M38;M49;M22;M59 |
| 908 | TFNVPMEKLKTLVA | M12;M81;M28;M29;M6;M45;M38;M49;M22;M59;M12;M81;M28; |
| | TAEAELAKNVS | M29;M6;M45;M38;M49;M22;M59 |
| 909 | PTTFHLDGEVITFDN | M40;M21;M16;M7;M30;M48;M50;M36;M49;M22;M40;M21;M16; |
| | LKTLLSLREV | M7;M30;M48;M50;M36;M49;M22 |
| 910 | AIILASFSASTSAFVE | M52;M28;M14;M16;M38;M6;M33;M8;M43;M42;M52;M28;M14; |
| | TVKGLDYKA | M16;M38;M6;M33;M8;M43;M42 |
| 911 | IAIILASFSASTSAFV | M52;M28;M14;M16;M38;M6;M33;M8;M43;M42;M52;M28;M14; |
| | ETVKGLDYK | M16;M38;M6;M33;M8;M43;M42 |
| 912 | EIVKFISTCACEIVG | M12;M78;M65;M29;M6;M74;M45;M79;M8;M20;M12;M78;M65; |
| | GQIVTCAKEI | M29;M6;M74;M45;M79;M8;M20 |
| 913 | TSSSKTPEEHFIETIS | M64;M40;M32;M14;M16;M30;M29;M6;M68;M8;M64;M40;M32; |
| | LAGSYKDWS | M14;M16;M30;M29;M6;M68;M8 |
| 914 | KTPEEHFIETISLAGS | M64;M40;M32;M14;M16;M30;M29;M6;M68;M8;M64;M40;M32; |
| | YKDWSYSGQ | M14;M16;M30;M29;M6;M68;M8 |
| 915 | SSSKTPEEHFIETISL | M64;M40;M32;M14;M16;M30;M29;M6;M68;M8;M64;M40;M32; |
| | AGSYKDWSY | M14;M16;M30;M29;M6;M68;M8 |
| 916 | SKTPEEHFIETISLAG | M64;M40;M32;M14;M16;M30;M29;M6;M68;M8;M64;M40;M32; |
| | SYKDWSYSG | M14;M16;M30;M29;M6;M68;M8 |
| 917 | SSKTPEEHFIETISLA | M64;M40;M32;M14;M16;M30;M29;M6;M68;M8;M64;M40;M32; |
| | GSYKDWSYS | M14;M16;M30;M29;M6;M68;M8 |
| 918 | AYITGGVVQLTSQW | M12;M54;M60;M48;M51;M42;M43;M49;M46;M9;M12;M54;M60; |
| | LTNIFGTVYEK | M48;M51;M42;M43;M49;M46;M9 |
| 919 | ATNNLVVMAYITGG | M12;M54;M60;M48;M51;M42;M43;M49;M46;M9;M12;M54;M60; |
| | VVQLTSQWLTN | M48;M51;M42;M43;M49;M46;M9 |
| 920 | TNNLVVMAYITGGV | M12;M54;M60;M48;M51;M42;M43;M49;M46;M9;M12;M54;M60; |
| | VQLTSQWLTNI | M48;M51;M42;M43;M49;M46;M9 |
| 921 | LVVMAYITGGVVQL | M12;M54;M60;M48;M51;M42;M43;M49;M46;M9;M12;M54;M60; |
| | TSQWLTNIFGT | M48;M51;M42;M43;M49;M46;M9 |
| 922 | MAYITGGVVQLTSQ | M12;M54;M60;M48;M51;M42;M43;M49;M46;M9;M12;M54;M60; |
| | WLTNIFGTVYE | M48;M51;M42;M43;M49;M46;M9 |
| 923 | YITGGVVQLTSQWL | M12;M54;M60;M48;M51;M42;M43;M49;M46;M9;M12;M54;M60; |
| | TNIFGTVYEKL | M48;M51;M42;M43;M49;M46;M9 |
| 924 | VMAYITGGVVQLTS | M12;M54;M60;M48;M51;M42;M43;M49;M46;M9;M12;M54;M60; |
| | QWLTNIFGTVY | M48;M51;M42;M43;M49;M46;M9 |
| 925 | VVMAYITGGVVQLT | M12;M54;M60;M48;M51;M42;M43;M49;M46;M9;M12;M54;M60; |
| | SQWLTNIFGTV | M48;M51;M42;M43;M49;M46;M9 |
| 926 | NLVVMAYITGGVV | M12;M54;M60;M48;M51;M42;M43;M49;M46;M9;M12;M54;M60; |
| | QLTSQWLTNIFG | M48;M51;M42;M43;M49;M46;M9 |
| 927 | NNLVVMAYITGGV | M12;M54;M60;M48;M51;M42;M43;M49;M46;M9;M12;M54;M60; |
| | VQLTSQWLTNIF | M48;M51;M42;M43;M49;M46;M9 |
| 928 | VVPGLPGTILRTTNG | M64;M75;M11;M24;M1;M6;M45;M8;M9;M23;M64;M75;M11;M24; |
| | DFLHFLPRVF | M1;M6;M45;M8;M9;M23 |
| 929 | GFVVPGLPGTILRTT | M64;M75;M11;M24;M1;M6;M45;M8;M9;M23;M64;M75;M11;M24; |
| | NGDFLHFLPR | M1;M6;M45;M8;M9;M23 |
| 930 | FVVPGLPGTILRTTN | M64;M75;M11;M24;M1;M6;M45;M8;M9;M23;M64;M75;M11;M24; |
| | GDFLHFLPRV | M1;M6;M45;M8;M9;M23 |
| 931 | VDAAKAYKDYLAS | M12;M48;M37;M57;M33;M69;M41;M8;M42;M39;M12;M48;M37; |
| | GGQPITNCVKML | M57;M33;M69;M41;M8;M42;M39 |
| 932 | SLRPDTRYVLMDGS | M25;M34;M7;M30;M48;M41;M43;M42;M39;M22;M25;M34;M7; |
| | IIQFPNTYLEG | M30;M48;M41;M43;M42;M39;M22 |
| 933 | HYKHITSKETLYCID | M21;M58;M10;M36;M43;M35;M46;M39;M9;M44;M21;M58;M10; |
| | GALLTKSSEY | M36;M43;M35;M46;M39;M9;M44 |
| 934 | SVQTFFKLVNKFLA | M16;M24;M34;M45;M57;M41;M42;M8;M43;M39;M16;M24;M34; |
| | LCADSIIIGGA | M45;M57;M41;M42;M8;M43;M39 |
| 935 | YESLRPDTRYVLMD | M25;M34;M7;M30;M48;M50;M41;M43;M42;M39;M25;M34;M7; |
| | GSIIQFPNTYL | M30;M48;M50;M41;M43;M42;M39 |
| 936 | AYESLRPDTRYVLM | M25;M34;M7;M30;M48;M50;M41;M43;M42;M39;M25;M34;M7; |
| | DGSIIQFPNTY | M30;M48;M50;M41;M43;M42;M39 |
| 937 | ESLRPDTRYVLMDG | M25;M34;M7;M30;M48;M50;M41;M43;M42;M39;M25;M34;M7; |
| | SIIQFPNTYLE | M30;M48;M50;M41;M43;M42;M39 |
| 938 | ILLAKDTTEAFEKM | M40;M16;M34;M30;M53;M36;M43;M42;M39;M22;M40;M16;M34; |
| | VSLLSVLLSMQ | M30;M53;M36;M43;M42;M39;M22 |
| 939 | VPGLPGTILRTTNGD | M64;M75;M11;M24;M6;M45;M8;M52;M9;M23;M64;M75;M11;M24; |
| | FLHFLPRVFS | M6;M45;M8;M52;M9;M23 |
| 940 | VQTFFKLVNKFLAL | M24;M34;M45;M57;M41;M67;M42;M8;M43;M39;M24;M34;M45; |
| | CADSIIIGGAK | M57;M41;M67;M42;M8;M43;M39 |
| 941 | DGEVITFDNLKTLLS | M32;M66;M31;M65;M30;M29;M48;M36;M68;M49;M32;M66;M31; |
| | LREVRTIKVF | M65;M30;M29;M48;M36;M68;M49 |
| 942 | VAKSHNIALIWNVK | M32;M28;M66;M1;M25;M6;M37;M72;M68;M59;M32;M28;M66;M1; |
| | DFMSLSEQLRK | M25;M6;M37;M72;M68;M59 |
| 943 | KFCLEASFNYLKSP | M4;M54;M19;M25;M60;M18;M41;M43;M42;M39;M4;M54;M19;M25; |
| | NFSKLINIIIW | M60;M18;M41;M43;M42;M39 |
| 944 | FCLEASFNYLKSPNF | M4;M54;M19;M25;M60;M18;M41;M43;M42;M39;M4;M54;M19;M25; |
| | SKLINIIIWF | M60;M18;M41;M43;M42;M39 |
| 945 | GKFCLEASFNYLKS | M4;M54;M19;M25;M60;M18;M41;M43;M42;M39;M4;M54;M19;M25; |
| | PNFSKLINIII | M60;M18;M41;M43;M42;M39 |
| 946 | VKFISTCACEIVGGQ | M28;M66;M12;M78;M65;M80;M29;M79;M50;M23;M28;M66;M12; |
| | IVTCAKEIKE | M78;M65;M80;M29;M79;M50;M23 |
| 947 | TLGVYDYLVSTQEF | M40;M31;M34;M30;M6;M33;M8;M43;M42;M47;M40;M31;M34; |
| | RYMNSQGLLPP | M30;M6;M33;M8;M43;M42;M47 |
| 948 | VTKIKPHNSHEGKT | M1;M34;M74;M37;M63;M41;M42;M43;M39;M22;M1;M34;M74; |
| | FYVLPNDDTLR | M37;M63;M41;M42;M43;M39;M22 |
| 949 | YKDYLASGGQPITN | M56;M12;M24;M10;M53;M37;M33;M8;M39;M23;M56;M12;M24; |
| | CVKMLCTHTGT | M10;M53;M37;M33;M8;M39;M23 |
| 950 | NGDSEVVLKKLKKS | M71;M13;M32;M28;M55;M1;M53;M33;M68;M17;M71;M13;M32; |
| | LNVAKSEFDRD | M28;M55;M1;M53;M33;M68;M17 |
| 951 | ANGDSEVVLKKLK | M71;M13;M32;M28;M55;M1;M53;M33;M68;M17;M71;M13;M32; |
| | KSLNVAKSEFDR | M28;M55;M1;M53;M33;M68;M17 |
| 952 | VETSNSFDVLKSED | M64;M34;M30;M6;M41;M42;M43;M46;M39;M22;M64;M34;M30; |
| | AQGMDNLACED | M6;M41;M42;M43;M46;M39;M22 |
| 953 | QTPFEIKLAKKFDTF | M23;M12;M58;M24;M10;M45;M57;M27;M17;M20;M23;M12;M58; |
| | NGECPNFVFP | M24;M10;M45;M57;M27;M17;M20 |
| 954 | TPFEIKLAKKFDTFN | M23;M12;M58;M24;M10;M45;M57;M27;M17;M20;M23;M12;M58; |
| | GECPNFVFPL | M24;M10;M45;M57;M27;M17;M20 |
| 955 | PGLPGTILRTTNGDF | M64;M75;M11;M24;M53;M6;M45;M52;M9;M23;M64;M75;M11; |
| | LHFLPRVFSA | M24;M53;M6;M45;M52;M9;M23 |
| 956 | SASTSAFVETVKGL | M52;M54;M16;M1;M60;M6;M38;M37;M35;M47;M52;M54;M16; |
| | DYKAFKQIVES | M1;M60;M6;M38;M37;M35;M47 |
| 957 | AGQKTYERHSLSHF | M64;M40;M16;M70;M31;M30;M15;M43;M39;M22;M64;M40;M16; |
| | VNLDNLRANNT | M70;M31;M30;M15;M43;M39;M22 |
| 958 | LLAKDTTEAFEKMV | M40;M16;M34;M30;M53;M69;M43;M42;M39;M22;M40;M16;M34; |
| | SLLSVLLSMQG | M30;M53;M69;M43;M42;M39;M22 |
| 959 | ETGLLMPLKAPKEII | M54;M24;M31;M60;M38;M48;M42;M27;M46;M39;M54;M24;M31; |
| | FLEGETLPTE | M60;M38;M48;M42;M27;M46;M39 |
| 960 | VEEAKTVLKKCKSA | M64;M21;M54;M16;M78;M65;M55;M60;M63;M9;M64;M21;M54; |
| | FYILPSIISNE | M16;M78;M65;M55;M60;M63;M9 |
| 961 | WALIISVTSNYSGVV | M64;M56;M32;M11;M14;M29;M38;M68;M22;M23;M64;M56;M32; |
| | TTVMFLARGI | M11;M14;M29;M38;M68;M22;M23 |
| 962 | MWALIISVTSNYSG | M64;M56;M32;M11;M14;M29;M38;M68;M22;M23;M64;M56;M32; |
| | VVTTVMFLARG | M11;M14;M29;M38;M68;M22;M23 |
| 963 | SEKSYELQTPFEIKL | M12;M58;M24;M10;M25;M34;M27;M17;M39;M23;M12;M58;M24; |
| | AKKFDTFNGE | M10;M25;M34;M27;M17;M39;M23 |
| 964 | KPHNSHEGKTFYVL | M31;M34;M74;M37;M63;M41;M42;M43;M39;M22;M31;M34;M74; |
| | PNDDTLRVEAF | M37;M63;M41;M42;M43;M39;M22 |
| 965 | PHNSHEGKTFYVLP | M31;M34;M74;M37;M63;M41;M42;M43;M39;M22;M31;M34;M74; |
| | NDDTLRVEAFE | M37;M63;M41;M42;M43;M39;M22 |
| 966 | AFYILPSIISNEKQEI | M56;M11;M24;M55;M10;M45;M50;M18;M9;M23;M56;M11;M24; |
| | LGTVSWNLR | M55;M10;M45;M50;M18;M9;M23 |
| 967 | FYILPSIISNEKQEIL | M56;M11;M24;M55;M10;M45;M50;M18;M9;M23;M56;M11;M24; |
| | GTVSWNLRE | M55;M10;M45;M50;M18;M9;M23 |
| 968 | LEASFNYLKSPNFSK | M4;M54;M25;M60;M74;M18;M41;M43;M42;M39;M4;M54;M25;M60; |
| | LINIIIWFLL | M74;M18;M41;M43;M42;M39 |
| 969 | EASFNYLKSPNFSKL | M4;M54;M25;M60;M74;M18;M41;M43;M42;M39;M4;M54;M25;M60; |
| | INIIIWFLLL | M74;M18;M41;M43;M42;M39 |
| 970 | LQAIASEFSSLPSYA | M21;M52;M28;M31;M41;M42;M68;M43;M39;M22;M21;M52;M28; |
| | AFATAQEAYE | M31;M41;M42;M68;M43;M39;M22 |
| 971 | TCGTTTLNGLWLDD | M56;M75;M24;M55;M65;M7;M53;M79;M9;M23;M56;M75;M24; |
| | VVYCPRHVICT | M55;M65;M7;M53;M79;M9;M23 |
| 972 | KSYELQTPFEIKLAK | M23;M12;M58;M24;M10;M25;M57;M27;M17;M20;M23;M12;M58; |
| | KFDTFNGECP | M24;M10;M25;M57;M27;M17;M20 |
| 973 | EKSYELQTPFEIKLA | M23;M12;M58;M24;M10;M25;M57;M27;M17;M20;M23;M12;M58; |
| | KKFDTFNGEC | M24;M10;M25;M57;M27;M17;M20 |
| 974 | IFGTVYEKLKPVLD | M32;M28;M31;M34;M67;M68;M39;M9;M47;M20;M32;M28;M31; |
| | WLEEKFKEGVE | M34;M67;M68;M39;M9;M47;M20 |
| 975 | LASGGQPITNCVKM | M4;M64;M21;M56;M58;M24;M10;M53;M22;M23;M4;M64;M21; |
| | LCTHTGTGQAI | M56;M58;M24;M10;M53;M22;M23 |
| 976 | YLASGGQPITNCVK | M4;M64;M21;M56;M58;M24;M10;M53;M22;M23;M4;M64;M21; |
| | MLCTHTGTGQA | M56;M58;M24;M10;M53;M22;M23 |
| 977 | TPVVQTIEVNSFSGY | M56;M60;M29;M38;M6;M50;M26;M9;M47;M23;M56;M60;M29; |
| | LKLTDNVYIK | M38;M6;M50;M26;M9;M47;M23 |
| 978 | VAAIFYLITPVHVMS | M56;M21;M34;M53;M18;M17;M35;M52;M39;M47;M56;M21;M34; |
| | KHTDFSSEII | M53;M18;M17;M35;M52;M39;M47 |
| 979 | LFVAAIFYLITPVHV | M56;M21;M34;M53;M18;M17;M35;M52;M39;M47;M56;M21;M34; |
| | MSKHTDFSSE | M53;M18;M17;M35;M52;M39;M47 |
| 980 | AAIFYLITPVHVMSK | M56;M21;M34;M53;M18;M17;M35;M52;M39;M47;M56;M21;M34; |
| | HTDFSSEIIG | M53;M18;M17;M35;M52;M39;M47 |
| 981 | FVAAIFYLITPVHVM | M56;M21;M34;M53;M18;M17;M35;M52;M39;M47;M56;M21;M34; |
| | SKHTDFSSEI | M53;M18;M17;M35;M52;M39;M47 |
| 982 | TGGVVQLTSQWLT | M4;M3;M45;M48;M51;M42;M43;M49;M46;M9;M59;M4;M3;M45; |
| | NIFGTVYEKLKP | M48;M51;M42;M43;M49;M46;M9;M59 |
| 983 | QWLTNIFGTVYEKL | M4;M32;M28;M31;M34;M3;M45;M47;M67;M39;M59;M4;M32;M28; |
| | KPVLDWLEEKF | M31;M34;M3;M45;M47;M67;M39;M59 |
| 984 | SQWLTNIFGTVYEK | M4;M32;M28;M31;M34;M3;M45;M47;M67;M39;M59;M4;M32;M28; |
| | LKPVLDWLEEK | M31;M34;M3;M45;M47;M67;M39;M59 |
| 985 | PGQGLNGYTVEEAK | M75;M19;M70;M60;M25;M45;M36;M63;M76;M39;M9;M75;M19; |
| | TVLKKCKSAFY | M70;M60;M25;M45;M36;M63;M76;M39;M9 |
| 986 | ALIWNVKDFMSLSE | M56;M28;M66;M19;M25;M6;M45;M37;M18;M72;M59;M56;M28; |
| | QLRKQIRSAAK | M66;M19;M25;M6;M45;M37;M18;M72;M59 |
| 987 | IILASFSASTSAFVET | M52;M28;M14;M16;M54;M38;M6;M33;M8;M43;M42;M52;M28; |
| | VKGLDYKAF | M14;M16;M54;M38;M6;M33;M8;M43;M42 |
| 988 | DGWEIVKFISTCACE | M23;M21;M29;M6;M45;M74;M72;M8;M49;M22;M20;M23;M21; |
| | IVGGQIVTCA | M29;M6;M45;M74;M72;M8;M49;M22;M20 |
| 989 | RDGWEIVKFISTCAC | M23;M21;M29;M6;M45;M74;M72;M8;M49;M22;M20;M23;M21; |
| | EIVGGQIVTC | M29;M6;M45;M74;M72;M8;M49;M22;M20 |
| 990 | VPTDNYITTYPGQG | M75;M14;M70;M26;M29;M48;M63;M41;M49;M15;M76;M75;M14; |
| | LNGYTVEEAKT | M70;M26;M29;M48;M63;M41;M49;M15;M76 |
| 991 | SFSASTSAFVETVKG | M52;M54;M14;M16;M1;M60;M6;M38;M37;M35;M47;M52;M54; |
| | LDYKAFKQIV | M14;M16;M1;M60;M6;M38;M37;M35;M47 |
| 992 | FSASTSAFVETVKGL | M52;M54;M14;M16;M1;M60;M6;M38;M37;M35;M47;M52;M54; |
| | DYKAFKQIVE | M14;M16;M1;M60;M6;M38;M37;M35;M47 |
| 993 | AIASEFSSLPSYAAF | M21;M12;M28;M70;M31;M6;M41;M42;M68;M52;M22;M21;M12; |
| | ATAQEAYEQA | M28;M70;M31;M6;M41;M42;M68;M52;M22 |
| 994 | CLEASFNYLKSPNFS | M4;M54;M19;M25;M60;M74;M18;M41;M43;M42;M39;M4;M54; |
| | KLINIIIWFL | M19;M25;M60;M74;M18;M41;M43;M42;M39 |
| 995 | KDYLASGGQPITNC | M4;M64;M56;M24;M10;M53;M37;M8;M39;M22;M23;M4;M64;M56; |
| | VKMLCTHTGTG | M24;M10;M53;M37;M8;M39;M22;M23 |
| 996 | TKIKPHNSHEGKTF | M31;M1;M34;M74;M37;M63;M41;M42;M43;M39;M22;M31;M1; |
| | YVLPNDDTLRV | M34;M74;M37;M63;M41;M42;M43;M39;M22 |
| 997 | IKPHNSHEGKTFYV | M31;M1;M34;M74;M37;M63;M41;M42;M43;M39;M22;M31;M1; |
| | LPNDDTLRVEA | M34;M74;M37;M63;M41;M42;M43;M39;M22 |
| 998 | KIKPHNSHEGKTFY | M31;M1;M34;M74;M37;M63;M41;M42;M43;M39;M22;M31;M1; |
| | VLPNDDTLRVE | M34;M74;M37;M63;M41;M42;M43;M39;M22 |
| 999 | PVETSNSFDVLKSED | M64;M25;M34;M30;M6;M41;M42;M43;M46;M39;M22;M64;M25; |
| | AQGMDNLACE | M34;M30;M6;M41;M42;M43;M46;M39;M22 |
| 1000 | KPVETSNSFDVLKSE | M64;M25;M34;M30;M6;M41;M42;M43;M46;M39;M22;M64;M25; |
| | DAQGMDNLAC | M34;M30;M6;M41;M42;M43;M46;M39;M22 |
| 1001 | WSTKPVETSNSFDV | M64;M25;M34;M30;M6;M41;M42;M43;M46;M39;M22;M64;M25; |
| | LKSEDAQGMDN | M34;M30;M6;M41;M42;M43;M46;M39;M22 |
| 1002 | STKPVETSNSFDVLK | M64;M25;M34;M30;M6;M41;M42;M43;M46;M39;M22;M64;M25; |
| | SEDAQGMDNL | M34;M30;M6;M41;M42;M43;M46;M39;M22 |
| 1003 | TKPVETSNSFDVLKS | M64;M25;M34;M30;M6;M41;M42;M43;M46;M39;M22;M64;M25; |
| | EDAQGMDNLA | M34;M30;M6;M41;M42;M43;M46;M39;M22 |
| 1004 | GQKTYERHSLSHFV | M64;M40;M16;M70;M31;M30;M42;M15;M43;M39;M22;M64;M40; |
| | NLDNLRANNTK | M16;M70;M31;M30;M42;M15;M43;M39;M22 |
| 1005 | AKDTTEAFEKMVSL | M40;M16;M34;M30;M53;M74;M69;M43;M42;M39;M22;M40;M16; |
| | LSVLLSMQGAV | M34;M30;M53;M74;M69;M43;M42;M39;M22 |
| 1006 | KDTTEAFEKMVSLL | M40;M16;M34;M30;M53;M74;M69;M43;M42;M39;M22;M40;M16; |
| | SVLLSMQGAVD | M34;M30;M53;M74;M69;M43;M42;M39;M22 |
| 1007 | LAKDTTEAFEKMVS | M40;M16;M34;M30;M53;M74;M69;M43;M42;M39;M22;M40;M16; |
| | LLSVLLSMQGA | M34;M30;M53;M74;M69;M43;M42;M39;M22 |
| 1008 | LVSSFLEMKSEKQV | M40;M16;M31;M25;M34;M30;M74;M41;M43;M42;M39;M40;M16; |
| | EQKIAEIPKEE | M31;M25;M34;M30;M74;M41;M43;M42;M39 |
| 1009 | TILRTTNGDFLHFLP | M75;M56;M58;M16;M24;M53;M45;M18;M17;M15;M52;M75;M56; |
| | RVFSAVGNIC | M58;M16;M24;M53;M45;M18;M17;M15;M52 |
| 1010 | YILPSIISNEKQEILG | M75;M56;M11;M24;M55;M10;M45;M50;M18;M9;M23;M75;M56; |
| | TVSWNLREM | M11;M24;M55;M10;M45;M50;M18;M9;M23 |
| 1011 | KEIKESVQTFFKLVN | M58;M16;M24;M53;M17;M35;M15;M42;M43;M39;M47;M58;M16; |
| | KFLALCADSI | M24;M53;M17;M35;M15;M42;M43;M39;M47 |
| 1012 | AKEIKESVQTFFKLV | M58;M16;M24;M53;M17;M35;M15;M42;M43;M39;M47;M58;M16; |
| | NKFLALCADS | M24;M53;M17;M35;M15;M42;M43;M39;M47 |
| 1013 | VFDKNLYDKLVSSF | M40;M16;M31;M70;M25;M34;M41;M43;M49;M42;M39;M40;M16; |
| | LEMKSEKQVEQ | M31;M70;M25;M34;M41;M43;M49;M42;M39 |
| 1014 | KGVEAVMYMGTLS | M4;M56;M40;M54;M16;M55;M1;M53;M3;M6;M37;M22;M4;M56; |
| | YEQFKKGVQIPC | M40;M54;M16;M55;M1;M53;M3;M6;M37;M22 |
| 1015 | TCASEYTGNYQCGH | M4;M52;M31;M1;M38;M3;M41;M42;M8;M43;M39;M9;M4;M52; |
| | YKHITSKETLY | M31;M1;M38;M3;M41;M42;M8;M43;M39;M9 |
| 1016 | LKGVEAVMYMGTL | M4;M56;M21;M40;M54;M55;M1;M53;M3;M6;M37;M22;M4;M56; |
| | SYEQFKKGVQIP | M21;M40;M54;M55;M1;M53;M3;M6;M37;M22 |
| 1017 | ITGGVVQLTSQWLT | M12;M54;M60;M3;M48;M45;M51;M42;M43;M49;M46;M9;M12; |
| | NIFGTVYEKLK | M54;M60;M3;M48;M45;M51;M42;M43;M49;M46;M9 |
| 1018 | WLTNIFGTVYEKLK | M4;M32;M28;M31;M34;M3;M45;M47;M67;M68;M39;M59;M4; |
| | PVLDWLEEKFK | M32;M28;M31;M34;M3;M45;M47;M67;M68;M39;M59 |
| 1019 | LTNIFGTVYEKLKPV | M4;M32;M28;M31;M34;M3;M45;M47;M67;M68;M39;M59;M4; |
| | LDWLEEKFKE | M32;M28;M31;M34;M3;M45;M47;M67;M68;M39;M59 |
| 1020 | QTTLKGVEAVMYM | M4;M56;M21;M40;M54;M55;M60;M53;M3;M6;M37;M22;M4;M56; |
| | GTLSYEQFKKGV | M21;M40;M54;M55;M60;M53;M3;M6;M37;M22 |
| 1021 | QQTTLKGVEAVMY | M4;M56;M21;M40;M54;M55;M60;M53;M3;M6;M37;M22;M4;M56; |
| | MGTLSYEQFKKG | M21;M40;M54;M55;M60;M53;M3;M6;M37;M22 |
| 1022 | QQQTTLKGVEAVM | M4;M56;M21;M40;M54;M55;M60;M53;M3;M6;M37;M22;M4;M56; |
| | YMGTLSYEQFKK | M21;M40;M54;M55;M60;M53;M3;M6;M37;M22 |
| 1023 | SFCAFAVDAAKAYK | M32;M21;M12;M28;M70;M48;M57;M33;M69;M63;M36;M8;M32; |
| | DYLASGGQPIT | M21;M12;M28;M70;M48;M57;M33;M69;M63;M36;M8 |
| 1024 | IALIWNVKDFMSLS | M56;M28;M66;M19;M1;M25;M6;M45;M37;M18;M72;M59;M56; |
| | EQLRKQIRSAA | M28;M66;M19;M1;M25;M6;M45;M37;M18;M72;M59 |
| 1025 | NIALIWNVKDFMSL | M56;M28;M66;M19;M1;M25;M6;M45;M37;M18;M72;M59;M56; |
| | SEQLRKQIRSA | M28;M66;M19;M1;M25;M6;M45;M37;M18;M72;M59 |
| 1026 | IASEFSSLPSYAAFA | M21;M12;M14;M28;M70;M31;M6;M41;M42;M68;M52;M22;M21; |
| | TAQEAYEQAV | M12;M14;M28;M70;M31;M6;M41;M42;M68;M52;M22 |
| 1027 | AVDAAKAYKDYLA | M12;M70;M48;M37;M57;M33;M69;M63;M41;M8;M42;M39;M12; |
| | SGGQPITNCVKM | M70;M48;M37;M57;M33;M69;M63;M41;M8;M42;M39 |
| 1028 | GVEFLRDGWEIVKFI | M4;M64;M21;M23;M29;M6;M45;M74;M48;M49;M22;M20;M4;M64; |
| | STCACEIVGG | M21;M23;M29;M6;M45;M74;M48;M49;M22;M20 |
| 1029 | EGVEFLRDGWEIVK | M4;M64;M21;M23;M29;M6;M45;M74;M48;M49;M22;M20;M4;M64; |
| | FISTCACEIVG | M21;M23;M29;M6;M45;M74;M48;M49;M22;M20 |
| 1030 | KVPTDNYITTYPGQ | M75;M14;M70;M16;M26;M29;M48;M63;M41;M49;M15;M76;M75; |
| | GLNGYTVEEAK | M14;M70;M16;M26;M29;M48;M63;M41;M49;M15;M76 |
| 1031 | NGDFLHFLPRVFSA | M64;M56;M12;M58;M16;M24;M1;M53;M45;M17;M46;M22;M64; |
| | VGNICYTPSKL | M56;M12;M58;M16;M24;M1;M53;M45;M17;M46;M22 |
| 1032 | LRPDTRYVLMDGSII | M25;M34;M7;M30;M48;M41;M43;M49;M42;M26;M39;M22;M25; |
| | QFPNTYLEGS | M34;M7;M30;M48;M41;M43;M49;M42;M26;M39;M22 |
| 1033 | QCGHYKHITSKETL | M40;M21;M52;M58;M31;M36;M41;M42;M8;M43;M39;M9;M40; |
| | YCIDGALLTKS | M21;M52;M58;M31;M36;M41;M42;M8;M43;M39;M9 |
| 1034 | YQCGHYKHITSKET | M40;M21;M52;M58;M31;M36;M41;M42;M8;M43;M39;M9;M40; |
| | LYCIDGALLTK | M21;M52;M58;M31;M36;M41;M42;M8;M43;M39;M9 |
| 1035 | NYQCGHYKHITSKE | M40;M21;M52;M58;M31;M36;M41;M42;M8;M43;M39;M9;M40; |
| | TLYCIDGALLT | M21;M52;M58;M31;M36;M41;M42;M8;M43;M39;M9 |
| 1036 | LPGTILRTTNGDFLH | M64;M75;M56;M11;M58;M16;M24;M53;M6;M45;M52;M9;M64; |
| | FLPRVFSAVG | M75;M56;M11;M58;M16;M24;M53;M6;M45;M52;M9 |
| 1037 | KFISTCACEIVGGQI | M56;M28;M66;M12;M78;M65;M80;M55;M10;M79;M50;M23;M56; |
| | VTCAKEIKES | M28;M66;M12;M78;M65;M80;M55;M10;M79;M50;M23 |
| 1038 | FISTCACEIVGGQIV | M56;M28;M66;M12;M78;M65;M80;M55;M10;M79;M50;M23;M56; |
| | TCAKEIKESV | M28;M66;M12;M78;M65;M80;M55;M10;M79;M50;M23 |
| 1039 | ILRTTNGDFLHFLPR | M75;M56;M58;M16;M24;M1;M53;M45;M18;M17;M15;M52;M75; |
| | VFSAVGNICY | M56;M58;M16;M24;M1;M53;M45;M18;M17;M15;M52 |
| 1040 | LRTTNGDFLHFLPR | M75;M56;M58;M16;M24;M1;M53;M45;M18;M17;M15;M52;M75; |
| | VFSAVGNICYT | M56;M58;M16;M24;M1;M53;M45;M18;M17;M15;M52 |
| 1041 | RTTNGDFLHFLPRV | M75;M56;M58;M16;M24;M1;M53;M45;M18;M17;M15;M52;M75; |
| | FSAVGNICYTP | M56;M58;M16;M24;M1;M53;M45;M18;M17;M15;M52 |
| 1042 | LPSIISNEKQEILGTV | M75;M56;M11;M24;M55;M10;M45;M50;M18;M8;M9;M23;M75; |
| | SWNLREMLA | M56;M11;M24;M55;M10;M45;M50;M18;M8;M9;M23 |
| 1043 | ILPSIISNEKQEILGT | M75;M56;M11;M24;M55;M10;M45;M50;M18;M8;M9;M23;M75; |
| | VSWNLREML | M56;M11;M24;M55;M10;M45;M50;M18;M8;M9;M23 |
| 1044 | HSLSHFVNLDNLRA | M64;M40;M16;M31;M30;M53;M42;M43;M15;M46;M39;M22;M64; |
| | NNTKGSLPINV | M40;M16;M31;M30;M53;M42;M43;M15;M46;M39;M22 |
| 1045 | SLSHFVNLDNLRAN | M64;M40;M16;M31;M30;M53;M42;M43;M15;M46;M39;M22;M64; |
| | NTKGSLPINVI | M40;M16;M31;M30;M53;M42;M43;M15;M46;M39;M22 |
| 1046 | TYERHSLSHFVNLD | M64;M40;M16;M70;M31;M30;M42;M43;M15;M46;M39;M22;M64; |
| | NLRANNTKGSL | M40;M16;M70;M31;M30;M42;M43;M15;M46;M39;M22 |
| 1047 | QKTYERHSLSHFVN | M64;M40;M16;M70;M31;M30;M42;M43;M15;M46;M39;M22;M64; |
| | LDNLRANNTKG | M40;M16;M70;M31;M30;M42;M43;M15;M46;M39;M22 |
| 1048 | KTYERHSLSHFVNL | M64;M40;M16;M70;M31;M30;M42;M43;M15;M46;M39;M22;M64; |
| | DNLRANNTKGS | M40;M16;M70;M31;M30;M42;M43;M15;M46;M39;M22 |
| 1049 | SAFYILPSIISNEKQEI | M56;M21;M11;M16;M24;M55;M10;M45;M50;M18;M9;M23;M56; |
| | LGTVSWNL | M21;M11;M16;M24;M55;M10;M45;M50;M18;M9;M23 |
| 1050 | PGTILRTTNGDFLHF | M75;M56;M58;M16;M24;M53;M45;M18;M17;M15;M52;M11;M75; |
| | LPRVFSAVGN | M56;M58;M16;M24;M53;M45;M18;M17;M15;M52;M11 |
| 1051 | GTILRTTNGDFLHFL | M75;M56;M58;M16;M24;M53;M45;M18;M17;M15;M52;M11;M75; |
| | PRVFSAVGNI | M56;M58;M16;M24;M53;M45;M18;M17;M15;M52;M11 |
| 1052 | KLVSSFLEMKSEKQ | M40;M16;M31;M70;M25;M34;M30;M41;M43;M49;M42;M39;M40; |
| | VEQKIAEIPKE | M16;M31;M70;M25;M34;M30;M41;M43;M49;M42;M39 |
| 1053 | DKLVSSFLEMKSEK | M40;M16;M31;M70;M25;M34;M30;M41;M43;M49;M42;M39;M40; |
| | QVEQKIAEIPK | M16;M31;M70;M25;M34;M30;M41;M43;M49;M42;M39 |
| 1054 | DKNLYDKLVSSFLE | M40;M16;M31;M70;M25;M34;M30;M41;M43;M49;M42;M39;M40; |
| | MKSEKQVEQKI | M16;M31;M70;M25;M34;M30;M41;M43;M49;M42;M39 |
| 1055 | NLYDKLVSSFLEMK | M40;M16;M31;M70;M25;M34;M30;M41;M43;M49;M42;M39;M40; |
| | SEKQVEQKIAE | M16;M31;M70;M25;M34;M30;M41;M43;M49;M42;M39 |
| 1056 | FDKNLYDKLVSSFL | M40;M16;M31;M70;M25;M34;M30;M41;M43;M49;M42;M39;M40; |
| | EMKSEKQVEQK | M16;M31;M70;M25;M34;M30;M41;M43;M49;M42;M39 |
| 1057 | LYDKLVSSFLEMKS | M40;M16;M31;M70;M25;M34;M30;M41;M43;M49;M42;M39;M40; |
| | EKQVEQKIAEI | M16;M31;M70;M25;M34;M30;M41;M43;M49;M42;M39 |
| 1058 | YDKLVSSFLEMKSE | M40;M16;M31;M70;M25;M34;M30;M41;M43;M49;M42;M39;M40; |
| | KQVEQKIAEIP | M16;M31;M70;M25;M34;M30;M41;M43;M49;M42;M39 |
| 1059 | KNLYDKLVSSFLEM | M40;M16;M31;M70;M25;M34;M30;M41;M43;M49;M42;M39;M40; |
| | KSEKQVEQKIA | M16;M31;M70;M25;M34;M30;M41;M43;M49;M42;M39 |
| 1060 | IKESVQTFFKLVNKF | M58;M16;M24;M53;M57;M17;M35;M15;M42;M43;M39;M47;M58; |
| | LALCADSIII | M16;M24;M53;M57;M17;M35;M15;M42;M43;M39;M47 |
| 1061 | EIKESVQTFFKLVNK | M58;M16;M24;M53;M57;M17;M35;M15;M42;M43;M39;M47;M58; |
| | FLALCADSII | M16;M24;M53;M57;M17;M35;M15;M42;M43;M39;M47 |
| 1062 | GTTTLNGLWLDDV | M71;M75;M56;M24;M10;M65;M55;M53;M7;M79;M9;M23;M71; |
| | VYCPRHVICTSE | M75;M56;M24;M10;M65;M55;M53;M7;M79;M9;M23 |
| 1063 | CGTTTLNGLWLDD | M71;M75;M56;M24;M10;M65;M55;M53;M7;M79;M9;M23;M71; |
| | VVYCPRHVICTS | M75;M56;M24;M10;M65;M55;M53;M7;M79;M9;M23 |
| 1064 | GVEAVMYMGTLSY | M4;M56;M40;M54;M16;M55;M1;M53;M3;M6;M37;M52;M22;M4; |
| | EQFKKGVQIPCT | M56;M40;M54;M16;M55;M1;M53;M3;M6;M37;M52;M22 |
| 1065 | ASEYTGNYQCGHY | M4;M40;M52;M31;M1;M34;M3;M41;M42;M8;M43;M39;M9;M4; |
| | KHITSKETLYCI | M40;M52;M31;M1;M34;M3;M41;M42;M8;M43;M39;M9 |
| 1066 | SEYTGNYQCGHYK | M4;M40;M52;M31;M1;M34;M3;M41;M42;M8;M43;M39;M9;M4; |
| | HITSKETLYCID | M40;M52;M31;M1;M34;M3;M41;M42;M8;M43;M39;M9 |
| 1067 | CASEYTGNYQCGH | M4;M40;M52;M31;M1;M38;M3;M41;M42;M8;M43;M39;M9;M4; |
| | YKHITSKETLYC | M40;M52;M31;M1;M38;M3;M41;M42;M8;M43;M39;M9 |
| 1068 | TLKGVEAVMYMGT | M4;M56;M21;M40;M54;M55;M1;M60;M53;M3;M6;M37;M22;M4; |
| | LSYEQFKKGVQI | M56;M21;M40;M54;M55;M1;M60;M53;M3;M6;M37;M22 |
| 1069 | TTLKGVEAVMYMG | M4;M56;M21;M40;M54;M55;M1;M60;M53;M3;M6;M37;M22;M4; |
| | TLSYEQFKKGVQ | M56;M21;M40;M54;M55;M1;M60;M53;M3;M6;M37;M22 |
| 1070 | KSHNIALIWNVKDF | M56;M28;M66;M19;M1;M25;M6;M45;M37;M18;M72;M68;M59; |
| | MSLSEQLRKQI | M56;M28;M66;M19;M1;M25;M6;M45;M37;M18;M72;M68;M59 |
| 1071 | SHNIALIWNVKDFM | M56;M28;M66;M19;M1;M25;M6;M45;M37;M18;M72;M68;M59; |
| | SLSEQLRKQIR | M56;M28;M66;M19;M1;M25;M6;M45;M37;M18;M72;M68;M59 |
| 1072 | HNIALIWNVKDFMS | M56;M28;M66;M19;M1;M25;M6;M45;M37;M18;M72;M68;M59; |
| | LSEQLRKQIRS | M56;M28;M66;M19;M1;M25;M6;M45;M37;M18;M72;M68;M59 |
| 1073 | EFLRDGWEIVKFIST | M4;M64;M21;M23;M29;M6;M45;M74;M48;M8;M49;M22;M20;M4; |
| | CACEIVGGQI | M64;M21;M23;M29;M6;M45;M74;M48;M8;M49;M22;M20 |
| 1074 | VEFLRDGWEIVKFIS | M4;M64;M21;M23;M29;M6;M45;M74;M48;M8;M49;M22;M20;M4; |
| | TCACEIVGGQ | M64;M21;M23;M29;M6;M45;M74;M48;M8;M49;M22;M20 |
| 1075 | QAIASEFSSLPSYAA | M21;M52;M12;M28;M70;M31;M6;M41;M42;M68;M43;M39;M22; |
| | FATAQEAYEQ | M21;M52;M12;M28;M70;M31;M6;M41;M42;M68;M43;M39;M22 |
| 1076 | GNYQCGHYKHITSK | M40;M21;M52;M58;M31;M34;M36;M41;M42;M8;M43;M39;M9; |
| | ETLYCIDGALL | M40;M21;M52;M58;M31;M34;M36;M41;M42;M8;M43;M39;M9 |
| 1077 | VGGQIVTCAKEIKES | M56;M66;M78;M65;M7;M53;M55;M10;M79;M50;M33;M8;M23; |
| | VQTFFKLVNK | M56;M66;M78;M65;M7;M53;M55;M10;M79;M50;M33;M8;M23 |
| 1078 | RHSLSHFVNLDNLR | M64;M40;M16;M70;M31;M30;M53;M42;M43;M15;M46;M39;M22; |
| | ANNTKGSLPIN | M64;M40;M16;M70;M31;M30;M53;M42;M43;M15;M46;M39;M22 |
| 1079 | ERHSLSHFVNLDNL | M64;M40;M16;M70;M31;M30;M53;M42;M43;M15;M46;M39;M22; |
| | RANNTKGSLPI | M64;M40;M16;M70;M31;M30;M53;M42;M43;M15;M46;M39;M22 |
| 1080 | YERHSLSHFVNLDN | M64;M40;M16;M70;M31;M30;M53;M42;M43;M15;M46;M39;M22; |
| | LRANNTKGSLP | M64;M40;M16;M70;M31;M30;M53;M42;M43;M15;M46;M39;M22 |
| 1081 | EEAKTVLKKCKSAF | M64;M21;M9;M16;M31;M78;M65;M60;M63;M42;M43;M39;M22; |
| | YILPSIISNEK | M64;M21;M9;M16;M31;M78;M65;M60;M63;M42;M43;M39;M22 |
| 1082 | TTTLNGLWLDDVV | M71;M75;M56;M24;M78;M65;M10;M55;M53;M7;M79;M9;M23; |
| | YCPRHVICTSED | M71;M75;M56;M24;M78;M65;M10;M55;M53;M7;M79;M9;M23 |
| 1083 | LRDGWEIVKFISTCA | M4;M64;M21;M23;M29;M6;M45;M74;M48;M72;M8;M49;M22;M20; |
| | CEIVGGQIVT | M4;M64;M21;M23;M29;M6;M45;M74;M48;M72;M8;M49;M22;M20 |
| 1084 | FLRDGWEIVKFISTC | M4;M64;M21;M23;M29;M6;M45;M74;M48;M72;M8;M49;M22;M20; |
| | ACEIVGGQIV | M4;M64;M21;M23;M29;M6;M45;M74;M48;M72;M8;M49;M22;M20 |
| 1085 | AKSHNIALIWNVKD | M56;M32;M28;M66;M19;M1;M25;M6;M45;M37;M18;M72;M68;M59; |
| | FMSLSEQLRKQ | M56;M32;M28;M66;M19;M1;M25;M6;M45;M37;M18;M72;M68;M59 |
| 1086 | FAVDAAKAYKDYL | M32;M12;M28;M70;M48;M37;M57;M33;M69;M63;M41;M8;M42;M39; |
| | ASGGQPITNCVK | M32;M12;M28;M70;M48;M37;M57;M33;M69;M63;M41;M8;M42;M39 |
| 1087 | TFHLDGEVITFDNLK | M40;M21;M32;M66;M16;M31;M7;M29;M30;M48;M36;M68;M49;M22; |
| | TLLSLREVRT | M40;M21;M32;M66;M16;M31;M7;M29;M30;M48;M36;M68;M49;M22 |
| 1088 | HLDGEVITFDNLKT | M40;M21;M32;M66;M16;M31;M7;M29;M30;M48;M36;M68;M49;M22; |
| | LLSLREVRTIK | M40;M21;M32;M66;M16;M31;M7;M29;M30;M48;M36;M68;M49;M22 |
| 1089 | FHLDGEVITFDNLKT | M40;M21;M32;M66;M16;M31;M7;M29;M30;M48;M36;M68;M49;M22; |
| | LLSLREVRTI | M40;M21;M32;M66;M16;M31;M7;M29;M30;M48;M36;M68;M49;M22 |
| 1090 | TTFHLDGEVITFDNL | M40;M21;M32;M16;M31;M7;M29;M30;M48;M50;M36;M68;M49;M22; |
| | KTLLSLREVR | M40;M21;M32;M16;M31;M7;M29;M30;M48;M50;M36;M68;M49;M22 |
| 1091 | ILASFSASTSAFVET | M52;M54;M28;M14;M16;M60;M6;M38;M33;M42;M8;M43;M35;M47; |
| | VKGLDYKAFK | M52;M54;M28;M14;M16;M60;M6;M38;M33;M42;M8;M43;M35;M47 |
| 1092 | ASFSASTSAFVETVK | M52;M54;M14;M16;M1;M60;M6;M38;M37;M42;M8;M43;M35;M47; |
| | GLDYKAFKQI | M52;M54;M14;M16;M1;M60;M6;M38;M37;M42;M8;M43;M35;M47 |
| 1093 | DDVVYCPRHVICTS | M64;M75;M56;M28;M66;M24;M55;M65;M6;M45;M72;M49;M59;M20; |
| | EDMLNPNYEDL | M64;M75;M56;M28;M66;M24;M55;M65;M6;M45;M72;M49;M59;M20 |
| 1094 | ESVQTFFKLVNKFL | M58;M16;M24;M34;M53;M45;M57;M41;M8;M17;M15;M35;M43;M39; |
| | ALCADSIIIGG | M58;M16;M24;M34;M53;M45;M57;M41;M8;M17;M15;M35;M43;M39 |
| 1095 | EIVGGQIVTCAKEIK | M56;M28;M66;M78;M65;M80;M7;M53;M55;M10;M79;M50;M8;M23; |
| | ESVQTFFKLV | M56;M28;M66;M78;M65;M80;M7;M53;M55;M10;M79;M50;M8;M23 |
| 1096 | CEIVGGQIVTCAKEI | M56;M28;M66;M78;M65;M80;M7;M53;M55;M10;M79;M50;M8;M23; |
| | KESVQTFFKL | M56;M28;M66;M78;M65;M80;M7;M53;M55;M10;M79;M50;M8;M23 |
| 1097 | TVLKKCKSAFYILPS | M40;M64;M21;M16;M31;M78;M65;M24;M30;M63;M42;M43;M39;M22; |
| | IISNEKQEIL | M40;M64;M21;M16;M31;M78;M65;M24;M30;M63;M42;M43;M39;M22 |
| 1098 | TTNGDFLHFLPRVFS | M64;M75;M56;M58;M16;M24;M1;M53;M45;M18;M17;M15;M52;M22; |
| | AVGNICYTPS | M64;M75;M56;M58;M16;M24;M1;M53;M45;M18;M17;M15;M52;M22 |
| 1099 | TGNYQCGHYKHITS | M4;M40;M21;M52;M58;M31;M1;M34;M3;M41;M42;M8;M43;M39;M9; |
| | KETLYCIDGAL | M4;M40;M21;M52;M58;M31;M1;M34;M3;M41;M42;M8;M43;M39;M9 |
| 1100 | YTGNYQCGHYKHIT | M4;M40;M21;M52;M58;M31;M1;M34;M3;M41;M42;M8;M43;M39;M9; |
| | SKETLYCIDGA | M4;M40;M21;M52;M58;M31;M1;M34;M3;M41;M42;M8;M43;M39;M9 |
| 1101 | EYTGNYQCGHYKHI | M4;M40;M21;M52;M58;M31;M1;M34;M3;M41;M42;M8;M43;M39;M9; |
| | TSKETLYCIDG | M4;M40;M21;M52;M58;M31;M1;M34;M3;M41;M42;M8;M43;M39;M9 |
| 1102 | FCAFAVDAAKAYK | M32;M21;M12;M28;M70;M48;M37;M57;M33;M69;M63;M41;M36;M8; |
| | DYLASGGQPITN | M39;M32;M21;M12;M28;M70;M48;M37;M57;M33;M69;M63;M41;M36; |
| | | M8;M39 |
| 1103 | CAFAVDAAKAYKD | M32;M21;M12;M28;M70;M48;M37;M57;M33;M69;M63;M41;M36;M8; |
| | YLASGGQPITNC | M39;M32;M21;M12;M28;M70;M48;M37;M57;M33;M69;M63;M41;M36; |
| | | M8;M39 |
| 1104 | AFAVDAAKAYKDY | M32;M21;M12;M28;M70;M48;M37;M57;M33;M69;M63;M41;M8;M42; |
| | LASGGQPITNCV | M39;M32;M21;M12;M28;M70;M48;M37;M57;M33;M69;M63;M41;M8; |
| | | M42;M39 |
| 1105 | LASFSASTSAFVETV | M52;M54;M28;M14;M16;M1;M60;M6;M38;M37;M42;M8;M43;M35; |
| | KGLDYKAFKQ | M47;M52;M54;M28;M14;M16;M1;M60;M6;M38;M37;M42;M8;M43; |
| | | M35;M47 |
| 1106 | LDGEVITFDNLKTLL | M40;M21;M32;M66;M16;M31;M7;M65;M29;M30;M48;M36;M68;M49; |
| | SLREVRTIKV | M22;M40;M21;M32;M66;M16;M31;M7;M65;M29;M30;M48;M36M68; |
| | | M49;M22 |
| 1107 | CACEIVGGQIVTCA | M56;M28;M66;M12;M78;M65;M80;M7;M53;M55;M10;M79;M50;M8; |
| | KEIKESVQTFF | M23;M56;M28;M66;M12;M78;M65;M80;M7;M53;M55;M10;M79;M50; |
| | | M8;M23 |
| 1108 | ISTCACEIVGGQIVT | M56;M28;M66;M12;M78;M65;M80;M7;M53;M55;M10;M79;M50;M8; |
| | CAKEIKESVQ | M23;M56;M28;M66;M12;M78;M65;M80;M7;M53;M55;M10;M79;M50; |
| | | M8;M23 |
| 1109 | STCACEIVGGQIVTC | M56;M28;M66;M12;M78;M65;M80;M7;M53;M55;M10;M79;M50;M8; |
| | AKEIKESVQT | M23;M56;M28;M66;M12;M78;M65;M80;M7;M53;M55;M10;M79;M50; |
| | | M8;M23 |
| 1110 | TCACEIVGGQIVTCA | M56;M28;M66;M12;M78;M65;M80;M7;M53;M55;M10;M79;M50;M8; |
| | KEIKESVQTF | M23;M56;M28;M66;M12;M78;M65;M80;M7;M53;M55;M10;M79;M50; |
| | | M8;M23 |
| 1111 | ACEIVGGQIVTCAK | M56;M28;M66;M12;M78;M65;M80;M7;M53;M55;M10;M79;M50;M8; |
| | EIKESVQTFFK | M23;M56;M28;M66;M12;M78;M65;M80;M7;M53;M55;M10;M79;M50; |
| | | M8;M23 |
| 1112 | SPFVMMSAPPAQYE | M40;M58;M16;M31;M19;M25;M34;M46;M41;M43;M35;M15;M52; |
| | LKHGTFTCASE | M39;M44;M40;M58;M16;M31;M19;M25;M34;M46;M41;M43;M35; |
| | | M15;M52;M39;M44 |
| 1113 | IVGGQIVTCAKEIKE | M56;M28;M66;M78;M65;M80;M7;M53;M55;M10;M79;M50;M33;M8; |
| | SVQTFFKLVN | M23;M56;M28;M66;M78;M65;M80;M7;M53;M55;M10;M79;M50; |
| | | M33;M8;M23 |
| 1114 | KTVLKKCKSAFYILP | M40;M64;M21;M16;M31;M78;M65;M60;M30;M24;M63;M42;M43; |
| | SIISNEKQEI | M39;M22;M40;M64;M21;M16;M31;M78;M65;M60;M30;M24;M63; |
| | | M42;M43;M39;M22 |
| 1115 | KESVQTFFKLVNKF | M58;M16;M24;M34;M53;M45;M57;M41;M17;M35;M15;M42;M43; |
| | LALCADSIIIG | M39;M47;M58;M16;M24;M34;M53;M45;M57;M41;M17;M35;M15; |
| | | M42;M43;M39;M47 |
| 1116 | AKTVLKKCKSAFYI | M40;M64;M21;M9;M16;M31;M78;M65;M60;M30;M63;M42;M43;M39; |
| | LPSIISNEKQE | M22;M40;M64;M21;M9;M16;M31;M78;M65;M60;M30;M63;M42;M43; |
| | | M39;M22 |
| 1117 | EAKTVLKKCKSAFY | M40;M64;M21;M9;M16;M31;M78;M65;M60;M30;M63;M42;M43;M39; |
| | ILPSIISNEKQ | M22;M40;M64;M21;M9;M16;M31;M78;M65;M60;M30;M63;M42;M43; |
| | | M39;M22 |
| 1118 | TTLNGLWLDDVVY | M71;M75;M56;M23;M28;M24;M78;M65;M10;M55;M53;M7;M79;M9; |
| | CPRHVICTSEDM | M20;M71;M75;M56;M23;M28;M24;M78;M65;M10;M55;M53;M7; |
| | | M79;M9;M20 |
| 1119 | STQEFRYMNSQGLL | M40;M16;M34;M30;M47;M48;M33;M18;M41;M42;M8;M43;M15;M46; |
| | PPKNSIDAFKL | M39;M44;M40;M16;M34;M30;M47;M48;M33;M18;M41;M42;M8; |
| | | M43;M15;M46;M39;M44 |
| 1120 | ESPFVMMSAPPAQY | M40;M58;M16;M31;M19;M25;M34;M46;M41;M43;M35;M15;M52; |
| | ELKHGTFTCAS | M39;M22;M44;M40;M58;M16;M31;M19;M25;M34;M46;M41;M43; |
| | | M35;M15;M52;M39;M22;M44 |
| 1121 | TNGDFLHFLPRVFS | M64;M75;M56;M12;M58;M16;M24;M1;M53;M45;M46;M18;M17; |
| | AVGNICYTPSK | M15;M52;M22;M64;M75;M56;M12;M58;M16;M24;M1;M53;M45; |
| | | M46;M18;M17;M15;M52;M22 |
| 1122 | KSAFYILPSIISNEKQ | M56;M21;M11;M22;M16;M31;M55;M10;M24;M45;M50;M18;M43; |
| | EILGTVSWN | M39;M9;M23;M56;M21;M11;M22;M16;M31;M55;M10;M24;M45; |
| | | M50;M18;M43;M39;M9;M23 |
| 1123 | TLNGLWLDDVVYC | M71;M75;M56;M23;M28;M24;M78;M65;M10;M55;M53;M7;M79; |
| | PRHVICTSEDML | M72;M9;M20;M71;M75;M56;M23;M28;M24;M78;M65;M10;M55; |
| | | M53;M7;M79;M72;M9;M20 |
| 1124 | VSTQEFRYMNSQGL | M40;M49;M16;M34;M30;M47;M48;M33;M18;M41;M42;M8;M43; |
| | LPPKNSIDAFK | M15;M46;M39;M44;M40;M49;M16;M34;M30;M47;M48;M33;M18; |
| | | M41;M42;M8;M43;M15;M46;M39;M44 |
| 1125 | VQQESPFVMMSAPP | M40;M58;M16;M31;M19;M25;M34;M46;M41;M42;M43;M35;M15; |
| | AQYELKHGTFT | M52;M39;M22;M44;M40;M58;M16;M31;M19;M25;M34;M46;M41; |
| | | M42;M43;M35;M15;M52;M39;M22;M44 |
| 1126 | KQATKYLVQQESPF | M40;M58;M16;M31;M19;M25;M34;M46;M41;M42;M43;M35;M15; |
| | VMMSAPPAQYE | M52;M39;M22;M44;M40;M58;M16;M31;M19;M25;M34;M46;M41; |
| | | M42;M43;M35;M15;M52;M39;M22;M44 |
| 1127 | QQESPFVMMSAPPA | M40;M58;M16;M31;M19;M25;M34;M46;M41;M42;M43;M35;M15; |
| | QYELKHGTFTC | M52;M39;M22;M44;M40;M58;M16;M31;M19;M25;M34;M46;M41; |
| | | M42;M43;M35;M15;M52;M39;M22;M44 |
| 1128 | QESPFVMMSAPPAQ | M40;M58;M16;M31;M19;M25;M34;M46;M41;M42;M43;M35;M15; |
| | YELKHGTFTCA | M52;M39;M22;M44;M40;M58;M16;M31;M19;M25;M34;M46;M41; |
| | | M42;M43;M35;M15;M52;M39;M22;M44 |
| 1129 | TKYLVQQESPFVM | M40;M58;M16;M31;M19;M25;M34;M30;M46;M41;M42;M43;M35; |
| | MSAPPAQYELKH | M15;M52;M39;M22;M44;M40;M58;M16;M31;M19;M25;M34;M30; |
| | | M46;M41;M42;M43;M35;M15;M52;M39;M22;M44 |
| 1130 | ATKYLVQQESPFVM | M40;M58;M16;M31;M19;M25;M34;M30;M46;M41;M42;M43;M35; |
| | MSAPPAQYELK | M15;M52;M39;M22;M44;M40;M58;M16;M31;M19;M25;M34;M30; |
| | | M46;M41;M42;M43;M35;M15;M52;M39;M22;M44 |
| 1131 | KYLVQQESPFVMMS | M40;M58;M16;M31;M19;M25;M34;M30;M46;M41;M42;M43;M35; |
| | APPAQYELKHG | M15;M52;M39;M22;M44;M40;M58;M16;M31;M19;M25;M34;M30; |
| | | M46;M41;M42;M43;M35;M15;M52;M39;M22;M44 |
| 1132 | QATKYLVQQESPFV | M40;M58;M16;M31;M19;M25;M34;M30;M46;M41;M42;M43;M35; |
| | MMSAPPAQYEL | M15;M52;M39;M22;M44;M40;M58;M16;M31;M19;M25;M34;M30; |
| | | M46;M41;M42;M43;M35;M15;M52;M39;M22;M44 |
| 1133 | LVQQESPFVMMSAP | M40;M58;M16;M31;M19;M25;M34;M30;M46;M41;M42;M43;M35; |
| | PAQYELKHGTF | M15;M52;M39;M22;M44;M40;M58;M16;M31;M19;M25;M34;M30; |
| | | M46;M41;M42;M43;M35;M15;M52;M39;M22;M44 |
| 1134 | YLVQQESPFVMMSA | M40;M58;M16;M31;M19;M25;M34;M30;M46;M41;M42;M43;M35; |
| | PPAQYELKHGT | M15;M52;M39;M22;M44;M40;M58;M16;M31;M19;M25;M34;M30; |
| | | M46;M41;M42;M43;M35;M15;M52;M39;M22;M44 |
| 1135 | LNGLWLDDVVYCP | M71;M75;M56;M23;M28;M66;M24;M78;M65;M10;M55;M53;M6; |
| | RHVICTSEDMLN | M79;M72;M9;M59;M20;M71;M75;M56;M23;M28;M66;M24;M78; |
| | | M65;M10;M55;M53;M6;M79;M72;M9;M59;M20 |
| 1136 | NGLWLDDVVYCPR | M71;M75;M56;M23;M28;M66;M24;M78;M65;M10;M55;M53;M6; |
| | HVICTSEDMLNP | M79;M72;M9;M59;M20;M71;M75;M56;M23;M28;M66;M24;M78; |
| | | M65;M10;M55;M53;M6;M79;M72;M9;M59;M20 |
| 1137 | LGVYDYLVSTQEFR | M40;M16;M31;M34;M30;M6;M45;M48;M33;M18;M47;M41;M42; |
| | YMNSQGLLPPK | M8;M43;M15;M46;M39;M44;M40;M16;M31;M34;M30;M6;M45;M48; |
| | | M33;M18;M47;M41;M42;M8;M43;M15;M46;M39;M44 |
| 1138 | LWLDDVVYCPRHVI | M71;M75;M56;M23;M28;M66;M24;M10;M65;M55;M53;M6;M45; |
| | CTSEDMLNPNY | M79;M72;M49;M9;M59;M20;M71;M75;M56;M23;M28;M66;M24; |
| | | M10;M65;M55;M53;M6;M45;M79;M72;M49;M9;M59;M20 |
| 1139 | KKCKSAFYILPSIISN | M40;M56;M21;M11;M22;M16;M31;M55;M10;M24;M30;M45;M50; |
| | EKQEILGTV | M18;M42;M43;M39;M9;M23;M40;M56;M21;M11;M22;M16;M31; |
| | | M55;M10;M24;M30;M45;M50;M18;M42;M43;M39;M9;M23 |
| 1140 | KCKSAFYILPSIISNE | M40;M56;M21;M11;M22;M16;M31;M55;M10;M24;M30;M45;M50; |
| | KQEILGTVS | M18;M42;M43;M39;M9;M23;M40;M56;M21;M11;M22;M16;M31; |
| | | M55;M10;M24;M30;M45;M50;M18;M42;M43;M39;M9;M23 |
| 1141 | CKSAFYILPSIISNEK | M40;M56;M21;M11;M22;M16;M31;M55;M10;M24;M30;M45;M50; |
| | QEILGTVSW | M18;M42;M43;M39;M9;M23;M40;M56;M21;M11;M22;M16;M31; |
| | | M55;M10;M24;M30;M45;M50;M18;M42;M43;M39;M9;M23 |
| 1142 | DYLVSTQEFRYMNS | M40;M49;M19;M16;M25;M34;M30;M47;M45;M48;M33;M18;M41; |
| | QGLLPPKNSID | M42;M8;M43;M15;M46;M39;M44;M40;M49;M19;M16;M25;M34; |
| | | M30;M47;M45;M48;M33;M18;M41;M42;M8;M43;M15;M46;M39;M44 |
| 1143 | LVSTQEFRYMNSQG | M40;M49;M19;M16;M25;M34;M30;M47;M45;M48;M33;M18;M41; |
| | LLPPKNSIDAF | M42;M8;M43;M15;M46;M39;M44;M40;M49;M19;M16;M25;M34; |
| | | M30;M47;M45;M48;M33;M18;M41;M42;M8;M43;M15;M46;M39;M44 |
| 1144 | YLVSTQEFRYMNSQ | M40;M49;M19;M16;M25;M34;M30;M47;M45;M48;M33;M18;M41; |
| | GLLPPKNSIDA | M42;M8;M43;M15;M46;M39;M44;M40;M49;M19;M16;M25;M34; |
| | | M30;M47;M45;M48;M33;M18;M41;M42;M8;M43;M15;M46;M39;M44 |
| 1145 | LDDVVYCPRHVICT | M71;M75;M56;M23;M64;M28;M66;M24;M10;M65;M55;M53;M6; |
| | SEDMLNPNYED | M45;M79;M72;M49;M9;M59;M20;M71;M75;M56;M23;M64;M28;M66; |
| | | M24;M10;M65;M55;M53;M6;M45;M79;M72;M49;M9;M59;M20 |
| 1146 | WLDDVVYCPRHVIC | M71;M75;M56;M23;M64;M28;M66;M24;M10;M65;M55;M53;M6; |
| | TSEDMLNPNYE | M45;M79;M72;M49;M9;M59;M20;M71;M75;M56;M23;M64;M28;M66; |
| | | M24;M10;M65;M55;M53;M6;M45;M79;M72;M49;M9;M59;M20 |
| 1147 | GLWLDDVVYCPRH | M71;M75;M56;M23;M28;M66;M24;M78;M65;M10;M55;M53;M6; |
| | VICTSEDMLNPN | M45;M79;M72;M49;M9;M59;M20;M71;M75;M56;M23;M28;M66; |
| | | M24;M78;M65;M10;M55;M53;M6;M45;M79;M72;M49;M9;M59;M20 |
| 1148 | LKKCKSAFYILPSIIS | M40;M64;M21;M56;M11;M22;M16;M31;M55;M10;M24;M30;M45; |
| | NEKQEILGT | M50;M18;M42;M43;M39;M9;M23;M40;M64;M21;M56;M11;M22; |
| | | M16;M31;M55;M10;M24;M30;M45;M50;M18;M42;M43;M39;M9;M23 |
| 1149 | VLKKCKSAFYILPSII | M40;M64;M21;M56;M22;M16;M31;M78;M55;M10;M30;M24;M45; |
| | SNEKQEILG | M50;M18;M42;M43;M39;M9;M23;M40;M64;M21;M56;M22;M16; |
| | | M31;M78;M55;M10;M30;M24;M45;M50;M18;M42;M43;M39;M9;M23 |
| 1150 | YDYLVSTQEFRYMN | M25;M34;M6;M48;M33;M18;M8;M43;M47;M44;M40;M16;M30;M45; |
| | SQGLLPPKNSI | M42;M39;M19;M41;M49;M15;M46;M25;M34;M6;M48;M33;M18; |
| | | M8;M43;M47;M44;M40;M16;M30;M45;M42;M39;M19;M41;M49; |
| | | M15;M46 |
| 1151 | VYDYLVSTQEFRYM | M25;M34;M6;M48;M33;M18;M8;M43;M47;M44;M40;M16;M30;M45; |
| | NSQGLLPPKNS | M42;M39;M19;M41;M49;M15;M46;M25;M34;M6;M48;M33;M18; |
| | | M8;M43;M47;M44;M40;M16;M30;M45;M42;M39;M19;M41;M49; |
| | | M15;M46 |
| 1152 | GVYDYLVSTQEFRY | M25;M34;M6;M48;M33;M18;M8;M43;M47;M44;M40;M16;M31; |
| | MNSQGLLPPKN | M30;M45;M42;M39;M19;M41;M49;M15;M46;M25;M34;M6;M48;M33; |
| | | M18;M8;M43;M47;M44;M40;M16;M31;M30;M45;M42;M39;M19; |
| | | M41;M49;M15;M46 |

TABLE 2B
MHC II-binding Epitopes from Orf9b

| Peptides | Alleles |
|---|---|
| TLNSLEDKAFQLTPIAVQMTKLATT | DQB1-0602_DQA1 0102; DRB4_0103; DRB1_0402; |
| | DRB1_0407; DPB1-0202_DPA1-0103; DPB1- |
| | 0201_DPA1-0103 |
| ALRLVDPQIQLAVTRMENAVGRDQN | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301; |
| | DRB1_0410 |
| RLVDPQIQLAVTRMENAVGRDQNNV | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301; |
| | DRB1_0410 |
| LRLVDPQIQLAVTRMENAVGRDQNN | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301; |
| | DRB1_0410 |
| PALRLVDPQIQLAVTRMENAVGRDQ | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301; |
| | DRB1_0410 |
| LVDPQIQLAVTRMENAVGRDQNNVG | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301; |
| | DRB1_0410 |
| VDPQIQLAVTRMENAVGRDQNNVGP | DQB1-0602_DQA1-0102; DPB1-0402_DPA1-0103; |
| | DRB1_0403; DRB1_0402; DRB1_0405; DRB1_0901; |
| | DRB1_0411; DRB1_0404; DQB1-0302_DQA1-0301 |
| EDKAFQLTPIAVQMTKLATTEELPD | DQB1-0602_DQA1-0102; DRB4_0103; DRB1_0402; |
| | DRB1_0407; DQB1-0202_DQA1-0201 |
| VGPKVYPIILRLGSPLSLNMARKTL | DPB1-0601_DPA1-0103; DQB1-0602_DQA1-0102; |
| | DRB1_0102; DRB1_0401; DPB1-1401_DPA1-0201; |
| | DPB1-0301_DPA1-0103; DPB1-0901_DPA1-0201; |
| | DPB1-1001_DPA1-0201; DRB1_1101 |
| NVGPKVYPIILRLGSPLSLNMARKT | DPB1-0601_DPA1-0103; DQB1-0602_DQA1-0102; |
| | DRB1_0102; DRB1_0401; DPB1-1401_DPA1-0201; |
| | DPB1-0301_DPA1-0103; DPB1-0901_DPA1-0201; |
| | DPB1-1001_DPA1-0201; DRB1_1101 |
| QNNVGPKVYPIILRLGSPLSLNMAR | DPB1-1301_DPA1-0201; DRB1_0102; DQB1- |
| | 0602_DQA1-0102; DRB1_0401; DPB1-1401_DPA1- |
| | 0201; DPB1-0301_DPA1-0103; DPB1-0901_DPA1- |
| | 0201; DPB1-1001_DPA1-0201; DRB1_1101 |
| DPQIQLAVTRMENAVGRDQNNVGPK | DQB1-0602_DQA1-0102; DPB1-0402_DPA1-0103; |
| | DRB1_0901; DRB1_0404; DQB1-0302_DQA1-0301 |
| NNVGPKVYPIILRLGSPLSLNMARK | DRB1_0102; DQB1-0602_DQA1-0102; DRB1_0401; |
| | DPB1-1401_DPA1-0201; DPB1-0301_DPA1-0103; |
| | DPB1-0901_DPA1-0201; DPB1-1001_DPA1-0201; |
| | DRB1_1101 |
| KTLNSLEDKAFQLTPIAVQMTKLAT | DPB1-0202_DPA1-0103; DPB1-0201_DPA1-0103; |
| | DRB4_0103; DRB1_0407 |
| NSLEDKAFQLTPIAVQMTKLATTEE | DQB1-0602_DQA1-0102; DRB4_0103; DRB1_0402; |
| | DRB1_0407; DPB1-0202_DPA1-0103 |
| LNSLEDKAFQLTPIAVQMTKLATTE | DQB1-0602_DQA1-0102; DRB4_0103; DRB1_0402; |
| | DRB1_0407; DPB1-0202_DPA1-0103 |
| LEDKAFQLTPIAVQMTKLATTEELP | DRB1_0402; DQB1-0602_DQA1-0102; DRB4_0103; |
| | DRB1_0407 |
| SLEDKAFQLTPIAVQMTKLATTEEL | DRB1_0402; DQB1-0602_DQA1-0102; DRB4_0103; |
| | DRB1_0407 |
| PLSLNMARKTLNSLEDKAFQLTPIA | DQB1-0201_DQA1-0201; DPB1-1101_DPA1-0201; |
| | DRB1_1202; DPB1-0202_DPA1-0103; DQB1- |
| | 0604_DQA1-0102; DPB1-0201_DPA1-0103; DQB1- |
| | 0202_DQA1-0201 |
| LSLNMARKTLNSLEDKAFQLTPIAV | DQB1-0201_DQA1-0201; DPB1-1101_DPA1-0201; |
| | DRB1_1202; DPB1-0202_DPA1-0103; DQB1- |
| | 0604_DQA1-0102; DPB1-0201_DPA1-0103; DQB1- |
| | 0202_DQA1-0201 |
| GPKVYPIILRLGSPLSLNMARKTLN | DRB1_0102; DPB1-0601_DPA1-0103; DQB1- |
| | 0602_DQA1-0102; DRB1_0401; DPB1-0301_DPA1- |
| | 0103; DRB1_1101 |
| MARKTLNSLEDKAFQLTPIAVQMTK | DQB1-0201_DQA1-0201; DRB1_1202; DRB1_0407; |
| | DPB1-0202_DPA1-0103; DQB1-0604_DQA1-0102; |
| | DPB1-0201_DPA1-0103; DQB1-0202_DQA1-0201 |
| ARKTLNSLEDKAFQLTPIAVQMTKL | DQB1-0201_DQA1-0201; DRB1_1202; DRB1_0407; |
| | DPB1-0202_DPA1-0103; DQB1-0604_DQA1-0102; |
| | DPB1-0201_DPA1-0103; DQB1-0202_DQA1-0201 |
| RDQNNVGPKVYPIILRLGSPLSLNM | DPB1-1301_DPA1-0201; DRB1_0102; DQB1- |
| | 0602_DQA1-0102; DRB1_0401; DPB1-1401_DPA1- |
| | 0201; DPB1-0901_DPA1-0201; DPB1-1001_DPA1- |
| | 0201; DRB1_1101 |
| GRDQNNVGPKVYPIILRLGSPLSLN | DPB1-1301_DPA1-0201; DRB1_0102; DQB1- |
| | 0602_DQA1-0102; DRB1_0401; DPB1-1401_DPA1- |
| | 0201; DPB1-0901_DPA1-0201; DPB1-1001_DPA1- |
| | 0201; DRB1_1101 |
| DQNNVGPKVYPIILRLGSPLSLNMA | DPB1-1301_DPA1-0201; DRB1_0102; DQB1- |
| | 0602_DQA1-0102; DRB1_0401; DPB1-1401_DPA1- |
| | 0201; DPB1-0901_DPA1-0201; DPB1-1001_DPA1- |
| | 0201; DRB1_1101 |
| HPALRLVDPQIQLAVTRMENAVGRD | DQB1-0602_DQA1-0102; DRB1_0403; DRB1_0402; |
| | DRB1_0405; DRB1_0901; DRB1_0411; DQB1- |
| | 0302_DQA1-0301; DRB1_0410 |
| LNMARKTLNSLEDKAFQLTPIAVQM | DQB1-0201_DQA1-0201; DRB1_1202; DPB1- |
| | 0202_DPA1-0103; DQB1-0604_DQA1-0102; DPB1- |
| | 0201_DPA1-0103; DQB1-0202_DQA1-0201 |
| NMARKTLNSLEDKAFQLTPIAVQMT | DQB1-0201_DQA1-0201; DRB1_1202; DPB1- |
| | 0202_DPA1-0103; DQB1-0604_DQA1-0102; DPB1- |
| | 0201_DPA1-0103; DQB1-0202_DQA1-0201 |
| SLNMARKTLNSLEDKAFQLTPIAVQ | DQB1-0201_DQA1-0201; DRB1_1202; DPB1- |
| | 0202_DPA1-0103; DQB1-0604_DQA1-0102; DPB1- |
| | 0201_DPA1-0103; DQB1-0202_DQA1-0201 |
| LTPIAVQMTKLATTEELPDEFVVVT | DRB1_0402; DRB4_0103; DQB1-0202_DQA1-0201 |
| KAFQLTPIAVQMTKLATTEELPDEF | DRB1_0402; DRB4_0103; DQB1-0202_DQA1-0201 |
| FQLTPIAVQMTKLATTEELPDEFVV | DRB1_0402; DRB4_0103; DQB1-0202_DQA1-0201 |
| AFQLTPIAVQMTKLATTEELPDEFV | DRB1_0402; DRB4_0103; DQB1-0202_DQA1-0201 |
| TPIAVQMTKLATTEELPDEFVVVTV | DRB1_0402; DRB4_0103; DQB1-0202_DQA1-0201 |
| QLTPIAVQMTKLATTEELPDEFVVV | DRB1_0402; DRB4_0103; DQB1-0202_DQA1-0201 |
| DKAFQLTPIAVQMTKLATTEELPDE | DRB1_0402; DRB4_0103; DQB1-0202_DQA1-0201 |
| VGRDQNNVGPKVYPIILRLGSPLSL | DPB1-1301_DPA1-0201; DQB1-0602_DQA1-0102; |
| | DPB1-1401_DPA1-0201; DPB1-0901_DPA1-0201; |
| | DPB1-1001_DPA1-0201; DRB1_1101 |
| AVGRDQNNVGPKVYPIILRLGSPLS | DPB1-1301_DPA1-0201; DQB1-0602_DQA1-0102; |
| | DPB1-1401_DPA1-0201; DPB1-0901_DPA1-0201; |
| | DPB1-1001_DPA1-0201; DRB1_1101 |
| RKTLNSLEDKAFQLTPIAVQMTKLA | DQB1-0201_DQA1-0201; DRB1_0407; DPB1- |
| | 0202_DPA1-0103; DQB1-0604_DQA1-0102; DPB1- |
| | 0201_DPA1-0103 |
| MHPALRLVDPQIQLAVTRMENAVGR | DRB1_0403; DRB1_0402; DRB1_0405; DRB1_0901; |
| | DRB1_0411; DQB1-0302_DQA1-0301; DRB1_0410 |
| EMHPALRLVDPQIQLAVTRMENAVG | DRB1_0403; DRB1_0402; DRB1_0405; DRB1_0901; |
| | DRB1_0411; DQB1-0302_DQA1-0301 |
| GSPLSLNMARKTLNSLEDKAFQLTP | DQB1-0201_DQA1-0201; DPB1-1101_DPA1-0201; |
| | DPB1-0202_DPA1-0103; DQB1-0604_DQA1-0102; |
| | DRB1_1202; DQB1-0202_DQA1-0201 |
| LRLGSPLSLNMARKTLNSLEDKAFQ | DQB1-0201_DQA1-0201; DPB1-1101_DPA1-0201; |
| | DQB1-0604_DQA1-0102; DRB1_1202; DQB1- |
| | 0202_DQA1-0201 |
| LGSPLSLNMARKTLNSLEDKAFQLT | DQB1-0201_DQA1-0201; DPB1-1101_DPA1-0201; |
| | DQB1-0604_DQA1-0102; DRB1_1202; DQB1- |
| | 0202_DQA1-0201 |
| RLGSPLSLNMARKTLNSLEDKAFQL | DQB1-0201_DQA1-0201; DPB1-1101_DPA1-0201; |
| | DQB1-0604_DQA1-0102; DRB1_1202; DQB1- |
| | 0202_DQA1-0201 |
| SPLSLNMARKTLNSLEDKAFQLTPI | DQB1-0201_DQA1-0201; DPB1-1101_DPA1-0201; |
| | DPB1-0202_DPA1-0103; DQB1-0604_DQA1-0102; |
| | DRB1_1202; DQB1-0202_DQA1-0201 |
| MDPKISEMHPALRLVDPQIQLAVTR | DRB5_0101; DRB1_1303; DRB1_0803; DRB5_0102; |
| | DRB1_1406 |
| VYPIILRLGSPLSLNMARKTLNSLE | DRB1_0102; DPB1-0601_DPA1-0103; DPB1- |
| | 0301_DPA1-0103; DRB1_0401 |
| KVYPIILRLGSPLSLNMARKTLNSL | DRB1_0102; DPB1-0601_DPA1-0103; DPB1- |
| | 0301_DPA1-0103; DRB1_0401 |
| PKVYPIILRLGSPLSLNMARKTLNS | DRB1_0102; DPB1-0601_DPA1-0103; DPB1- |
| | 0301_DPA1-0103; DRB1_0401 |
| DPKISEMHPALRLVDPQIQLAVTRM | DRB5_0101; DRB1_1303; DRB1_0803; DRB1_1406 |
| YPIILRLGSPLSLNMARKTLNSLED | DRB1_0102; DPB1-0301_DPA1-0103; DRB1_0401 |
| SEMHPALRLVDPQIQLAVTRMENAV | DQB1-0302_DQA1-0301; DRB1_0901 |
| PIAVQMTKLATTEELPDEFVVVTVK | DQB1-0202_DQA1-0201 |

TABLE 2C
(Table 2Ai and Table 2Aii Allele key)

| Allele | Alias | Allele | Alias | Allele | Alias | Allele | Alias |
|---|---|---|---|---|---|---|---|
| DPB1-0201 DPA1-0103 | M1 | DRB1 0404 | M22 | DRB1 1602 | M43 | DRB1 0403 | M64 |
| DPB1-0301 DPA1-0103 | M2 | DRB1 0406 | M23 | DRB3 0202 | M44 | DPB1-1001 | M65 |
| | | | | | | DPA1-0201 | |
| DPB1-0401 DPA1-0103 | M3 | DRB1 1401 | M24 | DQB1-0201 | M45 | DPB1-1101 | M66 |
| | | | | DQA1-0501 | | DPA1-0201 | |
| DPB1-0402 DPA1-0103 | M4 | DRB5 0102 | M25 | DRB1 0102 | M46 | DQB1-0604 | M67 |
| | | | | | | DQA1-0102 | |
| DPB1-0601 DPA1-0103 | M5 | DRB5 0202 | M26 | DRB1 0803 | M47 | DRB1 0410 | M68 |
| DQB1-0202 DQA1-0201 | M6 | DPB1-0101 | M27 | DRB1 1501 | M48 | DPB1-0501 | M69 |
| | | DPA1-0202 | | | | DPA1-0201 | |
| DRB1 0301 | M7 | DQB1-0302 | M28 | DRB1 1503 | M49 | DQB1-0502 | M70 |
| | | DQA1-0301 | | | | DQA1-0102 | |
| DRB1 0701 | M8 | DQB1-0303 | M29 | DRB1 0302 | M50 | DRB1 1302 | M71 |
| | | DQA1-0201 | | | | | |
| DRB1 1404 | M9 | DRB1 0401 | M30 | DRB4 0103 | M51 | DQB1-0603 | M72 |
| | | | | | | DQA1-0103 | |
| DRB1 1405 | M10 | DRB1 0405 | M31 | DRB1 0801 | M52 | DRB3 0201 | M73 |
| DRB3 0301 | M11 | DRB1 0411 | M32 | DRB1 1102 | M53 | DPB1-1301 | M74 |
| | | | | | | DPA1-0201 | |
| DQB1-0301 DQA1-0505 | M12 | DRB1 0901 | M33 | DRB1 1201 | M54 | DQB1-0401 | M75 |
| | | | | | | DQA1-0301 | |
| DQB1-0303 DQA1-0301 | M13 | DRB1 1001 | M34 | DRB1 1301 | M55 | DQB1-0402 | M76 |
| | | | | | | DQA1-0401 | |
| DQB1-0602 DQA1-0102 | M14 | DRB1 1303 | M35 | DRB1 1406 | M56 | DQB1-0601 | M77 |
| | | | | | | DQA1-0102 | |
| DRB1 0802 | M15 | DRB3 0101 | M36 | DPB1-0101 | M57 | DPB1-0901 | M78 |
| | | | | DPA1-0201 | | DPA1-0201 | |
| DRB1 1101 | M16 | DPB1-0202 | M37 | DRB1 0804 | M58 | DPB1-1701 | M79 |
| | | DPA1-0103 | | | | DPA1-0201 | |
| DRB1 1104 | M17 | DQB1-0601 | M38 | DQB1-0201 | M59 | DQB1-0301 | M80 |
| | | DQA1-0103 | | DQA1-0201 | | DQA1-0303 | |
| DRB1 1402 | M18 | DRB1 0101 | M39 | DRB1 1202 | M60 | DQB1-0301 | M81 |
| | | | | | | DQA1-0501 | |
| DRB5 0101 | M19 | DRB1 0407 | M40 | DPB1-0501 | M61 | DPB1-1401 | M82 |
| | | | | DPA1-0202 | | DPA1-0201 | |
| DPB1-0101 DPA1-0103 | M20 | DRB1 1502 | M41 | DQB1-0301 | M62 | DQB1-0502 | M83 |
| | | | | DQA1-0601 | | DQA1-0101 | |
| DRB1 0402 | M21 | DRB1 1601 | M42 | DQB1-0501 | M63 | | |
| | | | | DQA1-0101 | | | |

Selected peptides may be synthetically manufactured, prepared into a pharmaceutical composition and may be administered to a subject as an immunotherapeutic vaccine, where viral epitope peptide antigens stimulate T cells in vivo. Additionally, or alternatively, T cells may be from a subject, and stimulated in vitro with the selected viral epitope peptide antigens. Following adequate activation of the T cells, the activated T cells are administered to the subject as immunotherapy. Additionally, or alternatively, antigen presenting cells (APCs) may be from the subject, and the APCs are contacted with the peptides comprising viral epitope antigens in vitro. The peptides comprising the viral epitope antigen may be longer peptides, comprising 20-100 amino acids, or more. The longer peptides may comprise a plurality of epitope peptides presented as a concatemer. The longer peptides are taken up by APCs and processed for antigen presentation in an efficient manner. The viral antigen activated and viral antigen presenting APCs may be administered to the subject as personalized immunotherapy, for the APCs to activate T lymphocytes in vivo. Additionally, or alternatively, antigen presenting cells (APCs) may be from the subject, and the APCs are contacted with the peptides comprising viral epitope antigens in vitro; thereafter, the activated APCs are incubated with T cells from the subject to activate the T cells in vitro. The subject's T cells thus activated in vitro may be administered into the subject as personalized immunotherapy.

In some embodiments, the present disclosure disclosed herein also provides a large selection of viral epitope peptide and HLA pairs generated as an information library where the viral epitope: HLA pairs are ranked based on the binding affinity and presentation prediction value (PPV).

In some embodiments, the present disclosure disclosed herein also provides viral antigenic peptides comprising the epitopes that have been analyzed and selected as described in the steps above, and manufactured synthetically, for shelving and later use as off-the shelf immunotherapy reagents or products for treating coronavirus infection. In some embodiments, the manufactured peptides comprising the epitopes are solubilized in a suitable solution comprising a suitable excipient and may be frozen. In some embodiments, the manufactured peptides may be lyophilized and stored. In some embodiments, the manufactured peptides comprising the epitopes may be stored in a dry powder form. Upon determining an incoming subject's HLA repertoire, wherein the subject is in need for a therapeutic vaccine against a coronavirus, one or more viral antigenic peptides that can bind to the subject's HLA are recovered from the shelved products, mixed into a pharmaceutical composition and administered to the subject in need thereof.

In some embodiments, the viral genome may be analyzed to identify one or more B cell epitopes. In some embodiments, epitopes identified by analysis of the viral genome can be used for raising antibodies in a suitable host, such as a mammalian host, including but not limited to a mouse, a rat, a rabbit, sheep, pig, goat, lamb. In some embodiments, epitopes identified by analysis of the viral genome can be used for raising antibodies by recombinant technology.

In certain embodiments, the present disclosure provides a binding protein (e.g., an antibody or antigen-binding fragment thereof), or a T cell receptor (TCR), or a chimeric antigen receptor (CAR) capable of binding with a high affinity to a viral epitope peptide: human leukocyte antigen (HLA) complex. In some embodiments, the present disclosure provides a CAR that is capable of binding with a high affinity to a viral epitope peptide derived from the extracellular domain of a protein. In certain embodiments, an antigen-specific binding protein or TCR or CAR as described herein includes variant polypeptide species that have one or more amino acid substitutions, insertions, or deletions, provided that the binding protein retains or substantially retains its specific binding function.

In certain embodiments, a viral epitope specific binding protein, TCR or CAR is capable of (a) specifically binding to an antigen: HLA complex on a cell surface independent or in the absence of CD8. In certain embodiments, a viral epitope specific binding protein is a T cell receptor (TCR), a chimeric antigen receptor or an antigen-binding fragment of a TCR, any of which can be chimeric, humanized or human. In further embodiments, an antigen-binding fragment of the TCR comprises a single chain TCR (scTCR).

In certain embodiments, there is provided a composition comprising a viral epitope-specific binding protein or high affinity recombinant TCR according to any one of the above embodiments and a pharmaceutically acceptable carrier, diluent, or excipient.

Methods useful for isolating and purifying recombinantly produced soluble TCR, by way of example, can include obtaining supernatants from suitable host cell/vector systems that secrete the recombinant soluble TCR into culture media and then concentrating the media, for example using a commercially available filter or concentrator. Following concentration or filtration, the concentrate or filtrate, in some embodiments, can be purified, for example by application to a single suitable purification matrix or to a series of suitable matrices, such as an affinity matrix or an ion exchange resin. Alternatively or additionally, in some embodiments, one or more reverse phase HPLC steps may be employed to further purify a recombinant polypeptide. Such purification methods can also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art.

In one aspect, the viral protein may be a protein from a novel coronavirus, strain 2019 SARS-CoV 2 (available at NCBI Reference Sequence NC_045512.2), such as the proteins listed in Table 3.

TABLE 3
Viral Proteins

| | Length |
|---|---|
| Protein | (AAs) |
| orf1ab polyprotein | 7096 |
| orf1a polyprotein | 4405 |
| surface glycoprotein (S) | 1273 |
| nucleocapsid phosphoprotein (N) | 419 |
| ORF3a protein | 275 |
| membrane glycoprotein (M) | 222 |
| ORF7a protein | 121 |
| ORF8 protein | 121 |
| envelope protein (E) | 75 |
| ORF6 protein | 61 |
| ORF7b | 43 |
| ORF10 protein | 38 |
| ORF9b protein | 97 |

### Immunogenic and Vaccine Compositions

In one embodiment, provided herein is an immunogenic composition, e.g., a vaccine composition capable of raising a viral epitope-specific response (e.g., a humoral or cell-mediated immune response). In some embodiments, the immunogenic composition comprises viral epitope therapeutics (e.g., peptides, polynucleotides, TCR, CAR, cells containing TCR or CAR, dendritic cell containing polypeptide, dendritic cell containing polynucleotide, antibody, etc.) described herein corresponding to viral-specific viral epitope identified herein.

A person skilled in the art reading the present disclosure will be able to select viral epitope therapeutics by testing, for example, the generation of T cells in vitro as well as their efficiency and overall presence, the proliferation, affinity and expansion of certain T cells for certain peptides, and the functionality of the T cells, e.g. by analyzing the IFN-γ production or cell killing by T cells. The most efficient peptides can then combined as an immunogenic composition.

In one embodiment of the present disclosure the different viral epitope peptides and/or polypeptides are selected so that one immunogenic composition comprises viral epitope peptides and/or polypeptides capable of associating with different MHC molecules, such as different MHC class I molecule. In some embodiments, an immunogenic composition comprises viral epitope peptides and/or polypeptides capable of associating with the most frequently occurring MHC class I molecules. Hence immunogenic compositions described herein comprise different peptides capable of associating with at least 2, at least 3, or at least 4 MHC class I or class II molecules.

In one embodiment, an immunogenic composition described herein is capable of raising a specific cytotoxic T cells response, specific helper T cell response, or a B cell response.

In some embodiments, an immunogenic composition described herein can further comprise an adjuvant and/or a carrier. Examples of useful adjuvants and carriers are given herein below. Polypeptides and/or polynucleotides in the composition can be associated with a carrier such as e.g. a protein or an antigen-presenting cell such as e.g. a dendritic cell (DC) capable of presenting the peptide to a T cell or a B cell. In further embodiments, DC-binding peptides are used as carriers to target the viral epitope peptides and polynucleotides encoding the viral epitope peptides to dendritic cells (Sioud et al. FASEB J 27:3272-3283 (2013)).

In embodiments, the viral epitope polypeptides or polynucleotides can be provided as antigen presenting cells (e.g., dendritic cells) containing such polypeptides or polynucleotides. In other embodiments, such antigen presenting cells are used to stimulate T cells for use in patients.

In some embodiments, the antigen presenting cells are dendritic cells. In related embodiments, the dendritic cells are autologous dendritic cells that are pulsed with the non-mutated protein epitope peptide or nucleic acid. The viral epitope peptide can be any suitable peptide that gives rise to an appropriate T cell response. In some embodiments, the T cell is a CTL. In some embodiments, the T cell is a HTL.

Thus, one embodiment of the present disclosure an immunogenic composition containing at least one antigen presenting cell (e.g., a dendritic cell) that is pulsed or loaded with one or more viral epitope polypeptides or polynucleotides described herein. In embodiments, such APCs are autologous (e.g., autologous dendritic cells). Alternatively, peripheral blood mononuclear cells (PBMCs) from a patient can be loaded with viral epitope peptides or polynucleotides ex vivo. In related embodiments, such APCs or PBMCs are injected back into the patient.

The polynucleotide can be any suitable polynucleotide that is capable of transducing the dendritic cell, thus resulting in the presentation of a viral epitope peptide and induction of immunity. In one embodiment, the polynucleotide can be naked DNA that is taken up by the cells by passive loading. In another embodiment, the polynucleotide is part of a delivery vehicle, for example, a liposome, virus like particle, plasmid, or expression vector. In another embodiment, the polynucleotide is delivered by a vector-free delivery system, for example, high performance electroporation and high-speed cell deformation). In embodiments, such antigen presenting cells (APCs) (e.g., dendritic cells) or peripheral blood mononuclear cells (PBMCs) are used to stimulate a T cell (e.g., an autologous T cell). In related embodiments, the T cell is a CTL. In other related embodiments, the T cell is an HTL. Such T cells are then injected into the patient. In some embodiments, CTL is injected into the patient. In some embodiments, HTL is injected into the patient. In some embodiments, both CTL and HTL are injected into the patient. Administration of either therapeutic can be performed simultaneously or sequentially and in any order.

The pharmaceutical compositions (e.g., immunogenic compositions) described herein for therapeutic treatment are intended for parenteral, topical, nasal, oral or local administration. In some embodiments, the pharmaceutical compositions described herein are administered parenterally, e.g., intravenously, subcutaneously, intradermally, or intramuscularly. In some embodiments, described herein are compositions for parenteral administration which comprise a solution of the viral epitope peptides and immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of viral epitope peptides and polynucleotides described herein in the pharmaceutical formulations can vary widely, i.e., from less than about 0.1%, usually at or at least about 2% to as much as 20% to 50% or more by weight, and will be selected by fluid volumes, viscosities, etc., according to the particular mode of administration selected.

The viral epitope peptides and polynucleotides described herein can also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the DEC205 antigen, or with other therapeutic or immunogenic compositions. Thus, liposomes filled with a desired peptide or polynucleotide described herein can be directed to the site of lymphoid cells, where the liposomes then deliver the selected therapeutic/immunogenic polypeptide/polynucleotide compositions. Liposomes can be formed from standard vesicle-forming lipids, which generally include neutral and negatively charged phospholipids and a sterol, for example, cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a viral epitope polypeptides or polynucleotides to be incorporated into the liposome for cell surface determinants of the desired immune system cells. A liposome suspension containing a peptide can be administered intravenously, locally, topically, etc. in a dose which varies according to, inter alia, the manner of administration, the polypeptide or polynucleotide being delivered, and the stage of the disease being treated.

In some embodiments, viral epitope polypeptides and polynucleotides are targeted to antigen-presenting cells (e.g., in some embodiments dendritic cells). In one embodiment, the viral epitope polypeptides and polynucleotides are target to antigen-presenting cells (e.g., in some embodiments dendritic cells) using the markers DEC205, XCR1, CD197, CD80, CD86, CD123, CD209, CD273, CD283, CD289, CD184, CD85h, CD85j, CD85k, CD85d, CD85g, CD85a, TSLP receptor, or CD1a.

For solid compositions, conventional or nanoparticle nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more viral epitope polypeptides or polynucleotides described herein at a concentration of 25%-75%.

For aerosol administration, the viral epitope polypeptides or polynucleotides can be supplied in finely divided form along with a surfactant and propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, or 0.25-5%. The balance of the composition can be propellant. A carrier can also be included as desired, as with, e.g., lecithin for intranasal delivery.

Additional methods for delivering the viral epitope polynucleotides described herein are also known in the art. For instance, the nucleic acid can be delivered directly, as “naked DNA”. This approach is described, for instance, in Wolff et al., Science 247:1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

For therapeutic or immunization purposes, nucleic acids (e.g., in some embodiments mRNA) encoding one or more viral epitope peptides described herein, or peptide binding agents described herein can also be administered to patients. In some embodiments, nucleic acids (e.g., in some embodiments mRNA) encoding one or more viral epitope peptides described herein, or peptide binding agents described herein may be formulated in synthetic lipid nanoparticles. In some embodiments, the mRNA is a modified mRNA. In some embodiments, the mRNA is self-amplifying RNA. In a further embodiment, a mRNA, such as a self-amplifying RNA, is a part of a synthetic lipid nanoparticle formulation (Geall et al., Proc Natl Acad Sci USA. 109:14604-14609 (2012)).

In some embodiments, nucleic acids (e.g., in some embodiments mRNA) encoding one or more viral epitope peptides described herein or peptide binding agents described herein can be delivered complexed to cationic compounds, such as cationic lipids. In some embodiments, nucleic acids (e.g., in some embodiments mRNA) encoding one or more viral epitope peptides described herein or peptide binding agents described herein can be encapsulated in lipid nanoparticles. In some embodiments, lipid nanoparticles can comprise cationic lipid, non-cationic lipids (e.g., phospholipids and/or sterol), and/or PEG-lipids). In some embodiments, lipid nanoparticle can comprise cationic lipid, non-cationic lipids (e.g., phospholipids and/or sterol), and/or PEG-lipids) as described in WO2021/213924, the entire contents of which is incorporated herein by reference for purposes described herein. Lipid-mediated gene delivery methods are described, for instance, in WO 96/18372, WO 93/24640; Mannino & Gould-Fogerite, BioTechniques 6 (7): 682-691 (1988); U.S. Pat. No. 5,279,833; WO 91/06309; and Felgner et al., Proc. Natl. Acad. Sci. USA 84:7413-7414 (1987). As used in the present disclosure, “nanoparticle” refers to a particle having an average diameter suitable for parenteral administration. In various embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

Lipid nanoparticles (LNPs) (e.g., ribonucleic acid LNPs) described herein may exhibit a polydispersity index less than about 0.5, less than about 0.4, less than about 0.3, or about 0.2 or less. By way of example, the nucleic acid particles (e.g., ribonucleic acid particles) can exhibit a polydispersity index in a range of about 0.1 to about 0.3 or about 0.2 to about 0.3.

LNPs described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, an LNP (e.g., a ribonucleic acid LNP) described herein has an N/P ratio greater than or equal to 5. In some embodiments, an LNP (e.g., a ribonucleic acid LNP) described herein has an N/P ratio that is about 5, 6, 7, 8, 9, or 10. In some embodiments, an N/P ratio for an LNP (e.g., a ribonucleic acid LNP) described herein is from about 10 to about 50. In some embodiments, an N/P ratio for an LNP (e.g., a ribonucleic acid LNP) described herein is from about 10 to about 70. In some embodiments, an N/P ratio for a nucleic acid LNP (e.g., a ribonucleic acid LNP) described herein is from about 10 to about 120.

LNPs (e.g., ribonucleic acid LNPs) described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture can be microscopic, with particle sizes between 1 and 1000 nanometers. The mixture may be termed a colloid or a colloidal suspension. Sometimes the term “colloid” only refers to the particles in the mixture and not the entire suspension.

The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Different types of nucleic acid particles have been described previously to be suitable for delivery of nucleic acid in particulate form (e.g. Kaczmarek, J. C. et al., 2017, Genome Medicine 9, 60, which is herein incorporated by reference). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes LNPs comprising nucleic acid (e.g., a polyribonucleotide), at least one cationic or cationically ionizable lipid or lipid-like material, and/or at least one cationic polymer which associate with the nucleic acid (e.g., a polyribonucleotide) to form nucleic acid particles (e.g., ribonucleic acid particles, e.g., ribonucleic acid nanoparticles) and compositions comprising such particles. The LNPs (e.g., ribonucleic acid LNPs, e.g., ribonucleic acid LNPs) may comprise nucleic acid (e.g., a polyribonucleotide) which is complexed in different forms by non-covalent interactions to the LNPs.

Some embodiments described herein relate to compositions, methods and uses involving more than one, e.g., 2, 3, 4, 5, 6 or even more nucleic acid species (e.g., polyribonucleotide species).

In an LNP (e.g., ribonucleic acid LNP) formulation, it is possible that each nucleic acid species (e.g., polyribonucleotide species) is separately formulated as an individual nucleic acid LNP (e.g., ribonucleic acid LNP) formulation. In that case, each individual nucleic acid LNP (e.g., ribonucleic acid LNP) formulation will comprise one nucleic acid species (e.g., polyribonucleotide species). The individual LNP (e.g., ribonucleic acid LNP) formulations may be present as separate entities, e.g., in separate containers. Such formulations are obtainable by providing each nucleic acid species (e.g., polyribonucleotide species) separately (typically each in the form of a nucleic acid-containing solution) together with a particle-forming agent, thereby allowing the formation of particles. Respective particles will contain exclusively the specific nucleic acid species (e.g., polyribonucleotide species) that is being provided when the particles are formed (individual particulate formulations).

In some embodiments, a composition such as a pharmaceutical composition comprises more than one individual nucleic acid LNP (e.g., ribonucleic acid LNP) formulation. Respective pharmaceutical compositions are referred to as “mixed particulate formulations.” Mixed particulate formulations according to the present disclosure are obtainable by forming, separately, individual nucleic acid LNP (e.g., ribonucleic acid particle, e.g., ribonucleic acid nanoparticle) formulations, as described above, followed by a step of mixing of the individual nucleic acid LNP (e.g., ribonucleic acid particle, e.g., ribonucleic acid nanoparticle) formulations. By the step of mixing, a formulation comprising a mixed population of nucleic acid-containing particles is obtainable. Individual nucleic acid LNP (e.g., ribonucleic acid particle, e.g., ribonucleic acid nanoparticle) populations may be together in one container, comprising a mixed population of individual nucleic acid LNPs (e.g., ribonucleic acid LNP) formulations.

Alternatively, it is possible that different nucleic acid species (e.g., polyribonucleotide species) are formulated together as a “combined LNP formulation.” Such formulations are obtainable by providing a combined formulation (typically combined solution) of different nucleic acid species (e.g., polyribonucleotide species) species together with a particle-forming agent, thereby allowing the formation of particles. As opposed to a “mixed particulate formulation,” a “combined particulate formulation” will typically comprise particles that comprise more than one nucleic acid species (e.g., polyribonucleotide species) species. In a combined particulate composition different nucleic acid species (e.g., polyribonucleotide species) are typically present together in a single particle.

In certain embodiments, nucleic acids (e.g., polyribonucleotides), when present in provided nucleic acid LNPz (e.g., ribonucleic acid LNPs) are resistant in aqueous solution to degradation with a nuclease.

In some embodiments, lipid nanoparticles are liver-targeting lipid nanoparticles. In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### Cationic Polymeric Materials

Cationic polymers have been recognized as useful for developing such delivery vehicles, as reported in PCT App. Pub. No. WO 2021/001417, the entirety of which is incorporated herein by reference. As used herein, the term “polymer” refers to a composition comprising one or more molecules that comprise repeating units of one or more monomers. As used herein, “polymer,” “polymeric material,” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a normalized molecular weight (Mn), a weight average molecular weight (Mw), and/or a polydispersity index (PDI). In some embodiments, such repeat units can all be identical (a “homopolymer”); alternatively, in some cases, there can be more than one type of repeat unit present within the polymeric material (a “heteropolymer” or a “copolymer”). In some cases, a polymer is biologically derived, e.g., a biopolymer such as a protein. In some cases, additional moieties can also be present in the polymeric material, for example targeting moieties such as those described herein.

In some embodiments, a polymer utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, a polymeric material for use in accordance with the present disclosure is biocompatible. In certain embodiments, a biocompatible material is biodegradable, e.g., is able to degrade, chemically and/or biologically, within a physiological environment, such as within the body.

In certain embodiments, a polymeric material may be or comprise protamine or polyalkyleneimine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some embodiments, a polyalkyleneimine comprises polyethylenimine (PEI) and/or polypropylenimine. In some embodiments, a preferred polyalkyleneimine is polyethyleneimine (PEI). In some embodiments, the average molecular weight of PEI is preferably 0.75·102 to 107 Da, preferably 1000 to 105 Da, more preferably 10000 to 40000 Da, more preferably 15000 to 30000 Da, even more preferably 20000 to 25000 Da.

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g., by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials. Collectively, anionic and neutral polymeric materials are referred to herein as non-cationic polymeric materials.

### Lipid Particles

The terms “lipid” and “lipid-like material” are used herein to refer to molecules that comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

Lipid nanoparticles (also referred to as “lipid nanoparticles”) of the present disclosure comprise (i) a cationic lipid; (ii) a polymer-conjugated lipid, and (iii) one or more helper lipids. Lipid nanoparticles described herein are useful for the delivery of nucleic acid cargo (e.g., a polyribonucleotide) into the cell of a subject. In some embodiments, lipid nanoparticles comprising a nucleic acid (e.g., a polyribonucleotide) described herein are useful for causing increased expression of a protein (e.g., an antibody agent) in a subject. In some embodiments, lipid nanoparticles comprising a nucleic acid (e.g., a polyribonucleotide) described herein are useful for causing a pharmacological effect induced by expression of a protein in a subject. Lipid nanoparticles described herein are characterized by molar percentage (mol %) of components in the lipid nanoparticle. A mol % used in reference to a lipid component of a lipid nanoparticle is relative to the total other lipid components in the lipid nanoparticle.

a. Cationic Lipids

As described herein, lipid nanoparticles of the present disclosure comprise a cationic lipid. In some embodiments, a lipid nanoparticle for delivery of at least one polyribonucleotide described herein comprises a cationic lipid. A cationic lipid, as described herein, is a lipid that is positively charged or is ionizable, such that the cationic lipid will become positively charged when subjected to particular physiological conditions, e.g., a pH of about 7.4 or less, and can promote lipid aggregation. In some embodiments, a cationic lipid is a lipid comprising one or more amine groups which bear or are capable of bearing a positive charge.

In some embodiments, a cationic lipid may comprise a cationic, meaning positively charged, headgroup. In some embodiments, a cationic lipid may have a hydrophobic domain (e.g., one or more domains of a neutral lipid or an anionic lipid) provided that the cationic lipid has a net positive charge. In some embodiments, a cationic lipid comprises a polar headgroup, which in some embodiments may comprise one or more amine derivatives such as primary, secondary, and/or tertiary amines, quaternary ammonium, various combinations of amines, amidinium salts, or guanidine and/or imidazole groups as well as pyridinium, piperizine and amino acid headgroups such as lysine, arginine, ornithine and/or tryptophan. In some embodiments, a polar headgroup of a cationic lipid comprises one or more amine derivatives. In some embodiments, a polar headgroup of a cationic lipid comprises a quaternary ammonium. In some embodiments, a headgroup of a cationic lipid may comprise multiple cationic charges. In some embodiments, a headgroup of a cationic lipid comprises one cationic charge.

In some embodiments, a cationic lipid is selected from 1,2-dimyristoyl-sn-glycero-3-ethylphosphocholine (DMEPC); 2-dimyristoyl-3-trimethylammonium propane (DMTAP); dioleyl ether phosphatidylcholine (DOEPC); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N-(N′,N′dimethylaminoethane)-carbamoyl) cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

In some embodiments, a cationic lipid is one provided in WO2012/016184, which is incorporated herein by reference in its entirety. For example, in some embodiments, a cationic lipid is selected from 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, a cationic lipid is one provided in WO2020/219941, WO2017/075531, WO2016/176330, WO2017/049245, or U.S. Pat. No. 9,670,152, each of which is incorporated herein by reference in its entirety.

In some embodiments, a cationic lipid is a compound of Formula I:

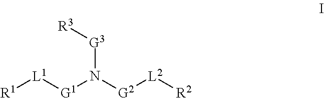

or a pharmaceutically acceptable salt thereof, wherein:
one of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}— or —NR^{a}C(O)O—, and the other of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}—, —NR^{a}C(O)O—, or a direct bond;
G¹ and G² are each independently unsubstituted C₁-C₁₂ alkylene or C1-C₁₂ alkenylene;
G³ is C₁-C₂₄ alkylene, C1-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C6-C₂₄ alkenyl;
R³ is H, OR⁵, CN, —C(O)OR⁴, —OC(O)R⁴ or -R⁵C(O)R⁴;
R⁴ is C₁-C₁₂ alkyl;
R⁵ is H or C₁-C₆ alkyl; and
x is 0, 1 or 2.

In some embodiments, one of L¹ or L² is —OC(O)— or —C(O)O—. In some embodiments, each of L¹ and L² is —OC(O)— or —C(O)O—.

In some embodiments, G¹ is C₁-C₁₂ alkylene. In some embodiments, G² is C₁-C₁₂ alkylene. In some embodiments G¹ and G² are each independently C₁-C₁₂ alkylene. In some embodiments G¹ and G² are each independently C5-C₁₂ alkylene.

In some embodiments, G³ is C₁-C₂₄ alkylene. In some embodiments, G³ is C₁-C₆ alkylene.

In some embodiments, R¹ and R² are each independently selected from:

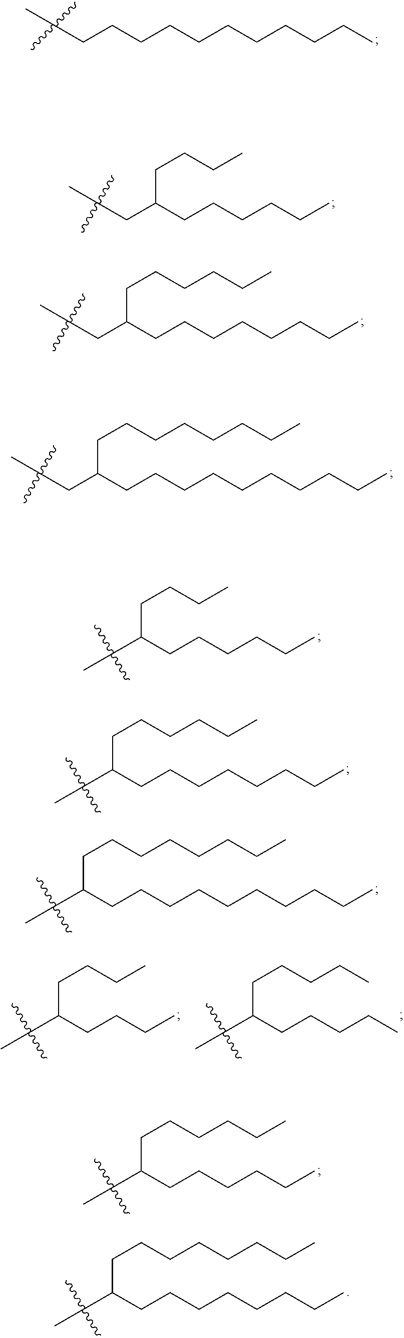

In some embodiments, R³ is OH.

In some embodiments, each of L¹ and L² is —OC(O)—, G¹ and G² are each independently C₅-C₁₂ alkylene, G³ is C₁-C₆ alkylene, R³ is OH, and R¹ and R² are each independently selected from:

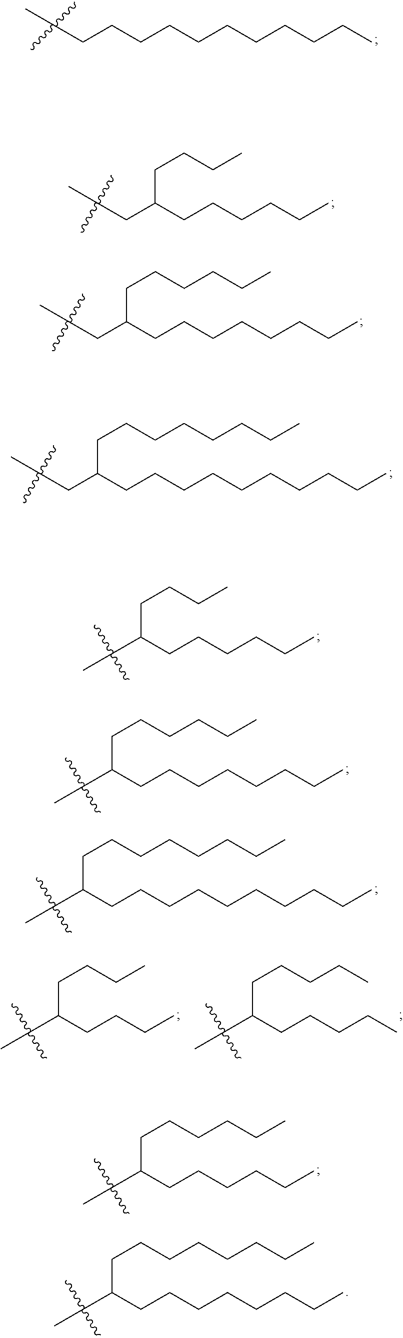

In some embodiments, a cationic lipid is a compound of Formula Ia or Ib

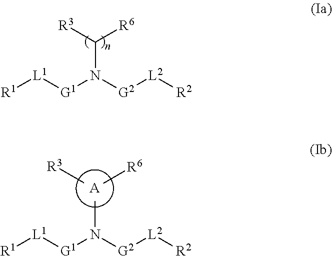

or a pharmaceutically acceptable salt thereof, where n is an integer from 1 to 15, A is C₃-C₈ cycloaliphatic, each R⁶ is independently selected from H, OH, and C₁-C₂₄ aliphatic, and wherein R¹, R², R³, L¹, L², G¹, and G² are as described in classes and subclasses herein, both singly and in combination.

In some embodiments, a positively charged lipid structure described herein may also include one or more other components that may be typically used in the formation of vesicles (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the lipid into the lipid assembly. Examples of sterols include cholesterol, cholesteryl hemisuccinate, cholesteryl sulfate, or any other derivatives of cholesterol. Preferably, the at least one cationic lipid comprises DMEPC and/or DOTMA.

In some embodiments, a cationic lipid is ionizable such that it can exist in a positively charged form or neutral form depending on pH. Such ionization of a cationic lipid can affect the surface charge of the lipid particle under different pH conditions, which in some embodiments may influence plasma protein absorption, blood clearance, and/or tissue distribution as well as the ability to form endosomolytic non-bilayer structures. Accordingly, in some embodiments, a cationic lipid may be or comprise a pH responsive lipid. In some embodiments a pH responsive lipid is a fatty acid derivative or other amphiphilic compound which is capable of forming a lyotropic lipid phase, and which has a pKa value between pH 5 and pH 7.5. This means that the lipid is uncharged at a pH above the pKa value and positively charged below the pKa value. In some embodiments, a pH responsive lipid may be used in addition to or instead of a cationic lipid for example by binding one or more polyribonucleotides to a lipid or lipid mixture at low pH. pH responsive lipids include, but are not limited to, 1,2-dioieyioxy-3-dimethylamino-propane (DODMA).

In some embodiments, a lipid nanoparticle may comprise one or more cationic lipids as described in WO 2017/075531 (e.g., as presented in Tables 1 and 3 therein) and WO 2018/081480 (e.g., as presented in Tables 1~4 therein), the entire contents of each of which are incorporated herein by reference for the purposes described herein.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKa of about 6.0-6.5 (e.g., in one embodiment with an apparent pKa of approximately 6.25), resulting in an essentially fully positively charged molecule at an acidic pH (e.g., pH 5). In some embodiments, such an amino lipid, when incorporated in lipid nanoparticle, can confer distinct physicochemical properties that regulate particle formation, cellular uptake, fusogenicity and/or endosomal release of polyribonucleotide(s). In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting lipid nanoparticle to a more neutral pH (e.g., pH 7.4) results in neutralization of the surface charge of the lipid nanoparticle. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders lipid nanoparticle comprising such an amino lipid fusogenic and allows the release of the RNA into the cytosol of the target cell.

In some embodiments, a cationic lipid that may be useful in accordance with the present disclosure has one of the structures set forth in Table B below:

TABLE B
Exemplary cationic lipids

| No. | Structure |
|---|---|
| I-1 | 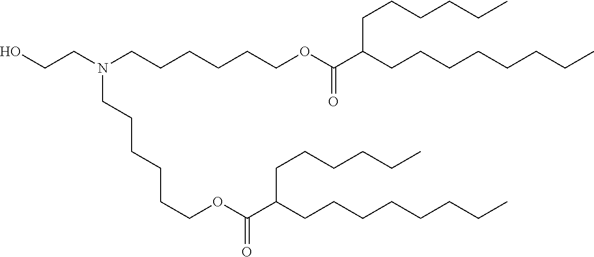 [SMILES: CCCCCCCCC(CCCCCC)C(=O)OCCCCCCN(CCO)CCCCCCOC(=O)C(CCCCCC)CCCCCCCC] |
| I-2 | 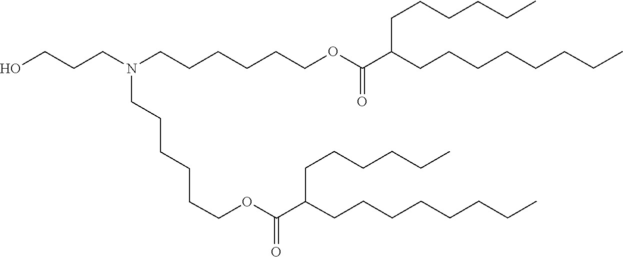 [SMILES: CCCCCCCCC(CCCCCC)C(=O)OCCCCCCN(CCCO)CCCCCCOC(=O)C(CCCCCC)CCCCCCCC] |
| I-3 | 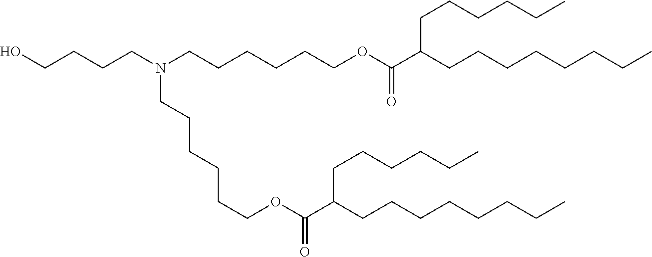 [SMILES: CCCCCCCCC(CCCCCC)C(=O)OCCCCCCN(CCCCO)CCCCCCOC(=O)C(CCCCCC)CCCCCCCC] |
| I-4 | 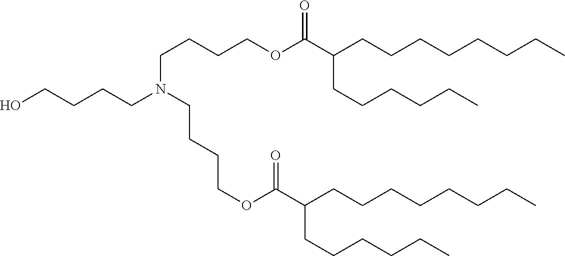 [SMILES: CCCCCCCCC(CCCCCC)C(=O)OCCCCN(CCCCO)CCCCOC(=O)C(CCCCCC)CCCCCCCC] |
| I-5 | 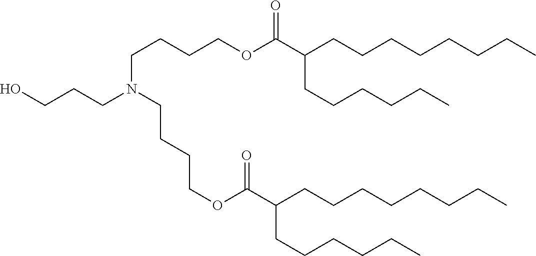 [SMILES: CCCCCCCCC(CCCCCC)C(=O)OCCCCN(CCCO)CCCCOC(=O)C(CCCCCC)CCCCCCCC] |
| I-6 | 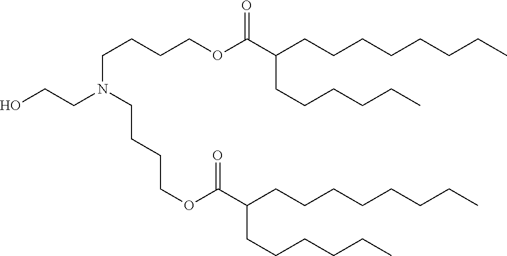 [SMILES: CCCCCCCCC(CCCCCC)C(=O)OCCCCN(CCO)CCCCOC(=O)C(CCCCCC)CCCCCCCC] |
| I-7 | 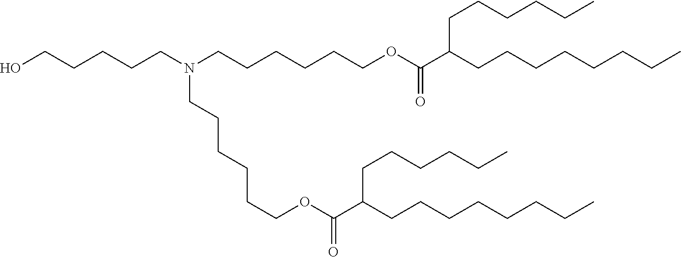 [SMILES: CCCCCCCCC(CCCCCC)C(=O)OCCCCCCN(CCCCCO)CCCCCCOC(=O)C(CCCCCC)CCCCCCCC] |
| I-8 | 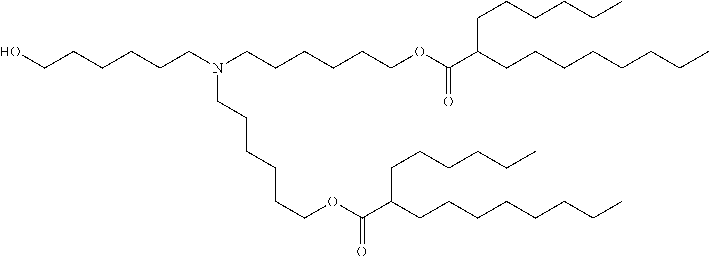 [SMILES: CCCCCCCCC(CCCCCC)C(=O)OCCCCCCN(CCCCCCO)CCCCCCOC(=O)C(CCCCCC)CCCCCCCC] |
| I-9 | 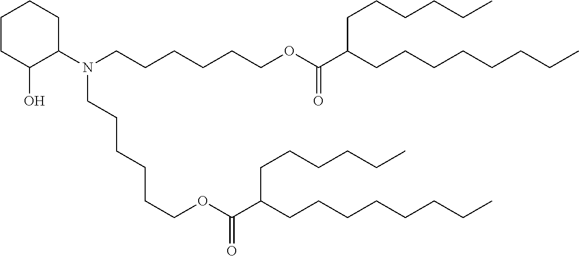 [SMILES: CCCCCCCCC(CCCCCC)C(=O)OCCCCCCN(CCCCCCOC(=O)C(CCCCCC)CCCCCCCC)C1CCCCC1O] |
| I-10 | 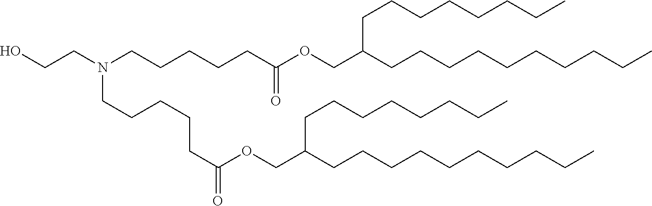 [SMILES: CCCCCCCCCCC(CCCCCCCC)COC(=O)CCCCCN(CCO)CCCCCC(=O)OCC(CCCCCCCC)CCCCCCCCCC] |
| I-11 | 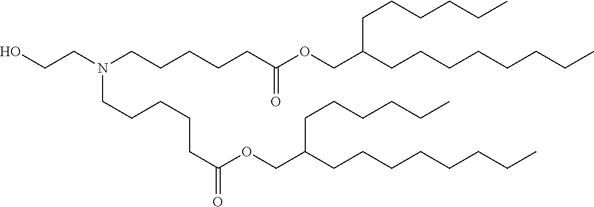 [SMILES: CCCCCCCCC(CCCCCC)COC(=O)CCCCCN(CCO)CCCCCC(=O)OCC(CCCCCC)CCCCCCCC] |
| I-12 | 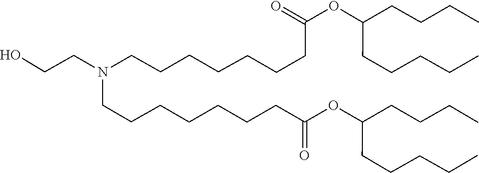 [SMILES: CCCCCC(CCCC)OC(=O)CCCCCCCN(CCO)CCCCCCCC(=O)OC(CCCC)CCCCC] |
| I-13 | 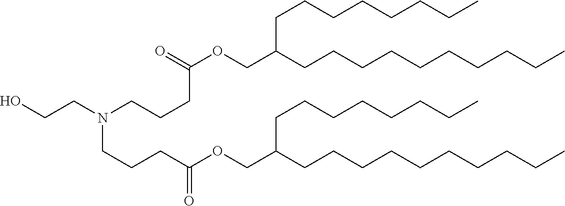 [SMILES: CCCCCCCCCCC(CCCCCCCC)COC(=O)CCCN(CCO)CCCC(=O)OCC(CCCCCCCC)CCCCCCCCCC] |
| I-14 | 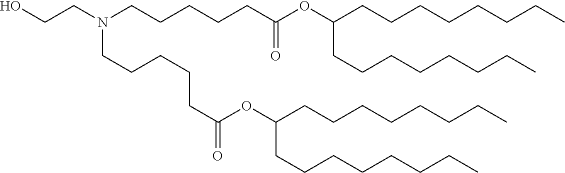 [SMILES: CCCCCCCCC(CCCCCCCC)OC(=O)CCCCCN(CCO)CCCCCC(=O)OC(CCCCCCCC)CCCCCCCC] |
| I-15 | 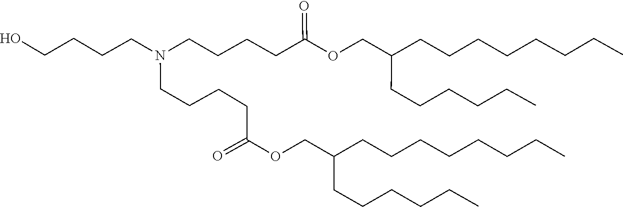 [SMILES: CCCCCCCCC(CCCCCC)COC(=O)CCCCN(CCCCO)CCCCC(=O)OCC(CCCCCC)CCCCCCCC] |
| I-16 | 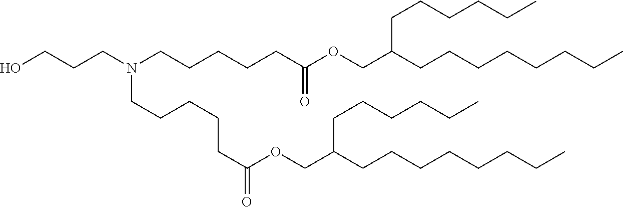 [SMILES: CCCCCCCCC(CCCCCC)COC(=O)CCCCCN(CCCO)CCCCCC(=O)OCC(CCCCCC)CCCCCCCC] |
| I-17 | 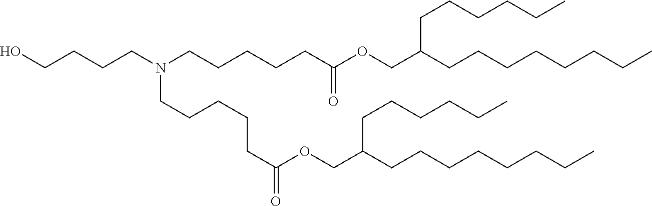 [SMILES: CCCCCCCCC(CCCCCC)COC(=O)CCCCCN(CCCCO)CCCCCC(=O)OCC(CCCCCC)CCCCCCCC] |
| I-18 | 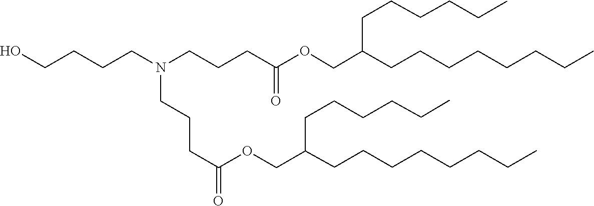 [SMILES: CCCCCCCCC(CCCCCC)COC(=O)CCCN(CCCCO)CCCC(=O)OCC(CCCCCC)CCCCCCCC] |
| I-19 | 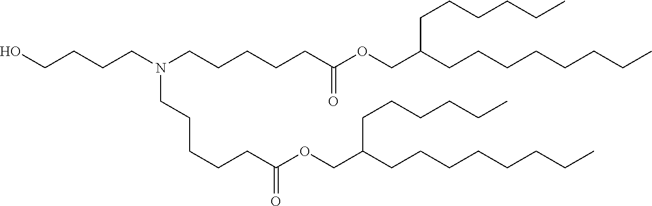 [SMILES: CCCCCCCCC(CCCCCC)COC(=O)CCCCCN(CCCCO)CCCCCC(=O)OCC(CCCCCC)CCCCCCCC] |
| I-20 | 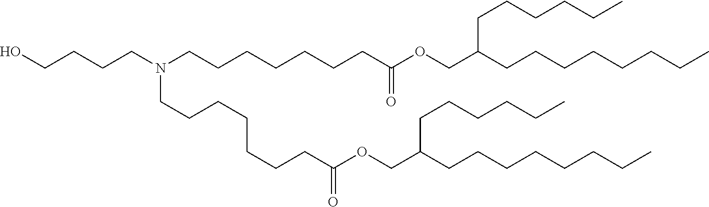 [SMILES: CCCCCCCCC(CCCCCC)COC(=O)CCCCCCCN(CCCCO)CCCCCCCC(=O)OCC(CCCCCC)CCCCCCCC] |
| I-21 | 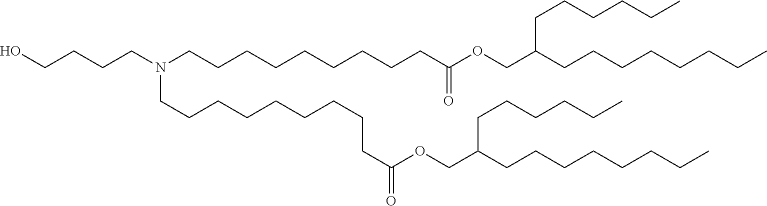 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 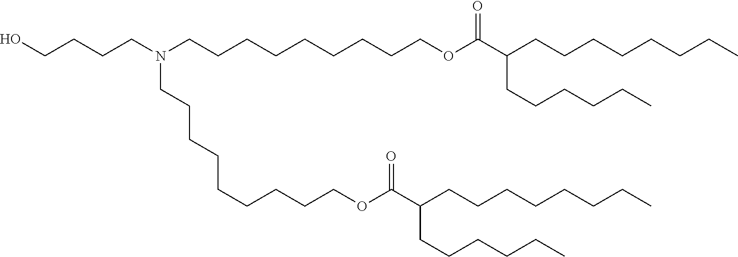 [SMILES: CCCCCCCCC(CCCCCC)C(=O)OCCCCCCCCCN(CCCCO)CCCCCCCCCOC(=O)C(CCCCCC)CCCCCCCC] |
| I-23 | 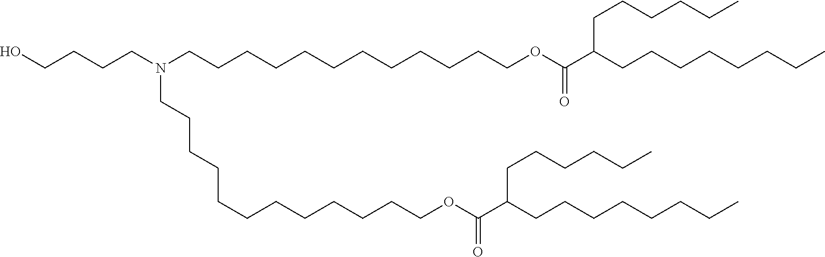 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 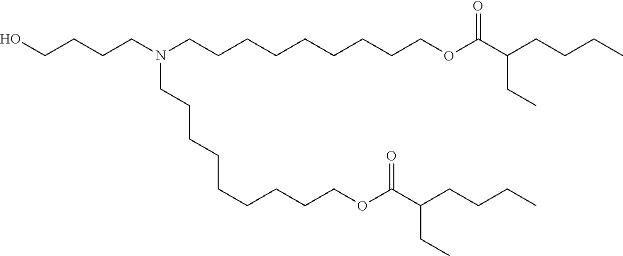 [SMILES: CCCCC(CC)C(=O)OCCCCCCCCCN(CCCCO)CCCCCCCCCOC(=O)C(CC)CCCC] |
| I-25 | 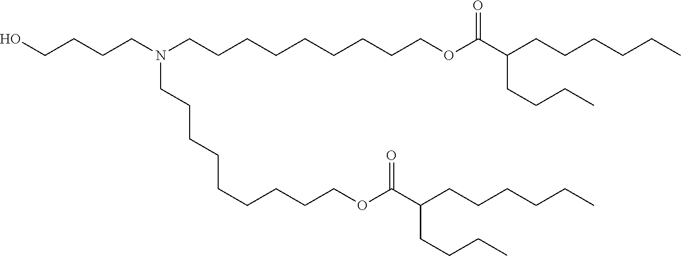 [SMILES: CCCCCCC(CCCC)C(=O)OCCCCCCCCCN(CCCCO)CCCCCCCCCOC(=O)C(CCCC)CCCCCC] |
| I-26 | 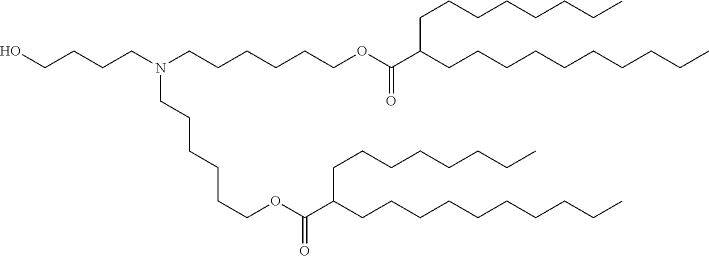 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 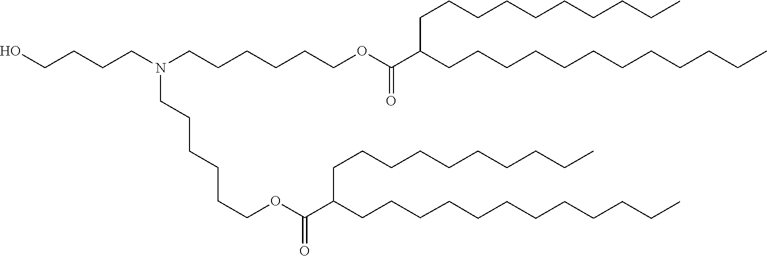 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 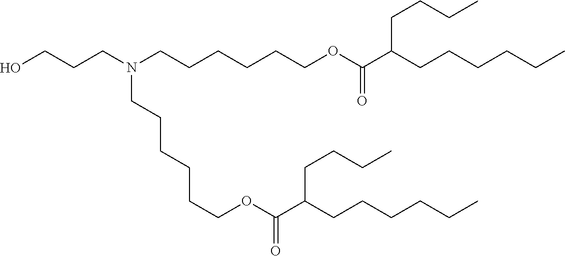 [SMILES: CCCCCCC(CCCC)C(=O)OCCCCCCN(CCCO)CCCCCCOC(=O)C(CCCC)CCCCCC] |
| I-29 | 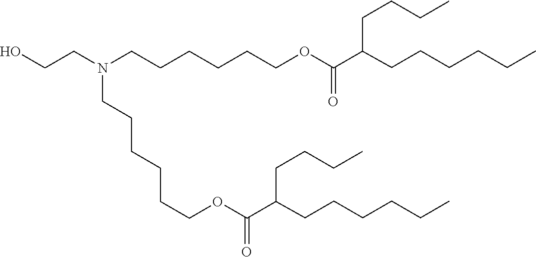 [SMILES: CCCCCCC(CCCC)C(=O)OCCCCCCN(CCO)CCCCCCOC(=O)C(CCCC)CCCCCC] |
| I-30 | 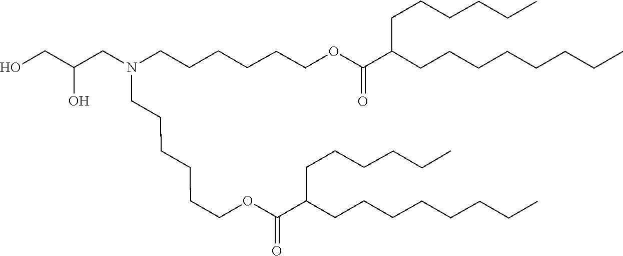 [SMILES: CCCCCCCCC(CCCCCC)C(=O)OCCCCCCN(CCCCCCOC(=O)C(CCCCCC)CCCCCCCC)CC(O)CO] |
| I-31 | 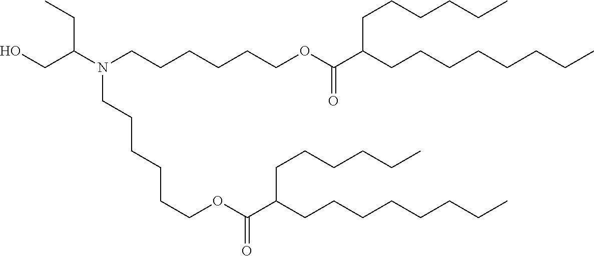 [SMILES: CCCCCCCCC(CCCCCC)C(=O)OCCCCCCN(CCCCCCOC(=O)C(CCCCCC)CCCCCCCC)C(CC)CO] |
| I-32 | 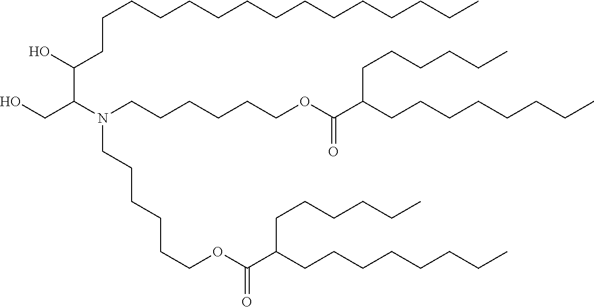 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 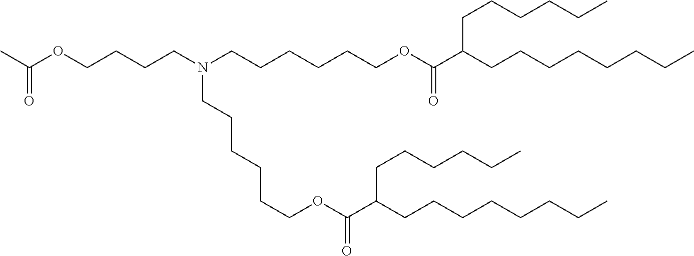 [SMILES: CCCCCCCCC(CCCCCC)C(=O)OCCCCCCN(CCCCCCOC(=O)C(CCCCCC)CCCCCCCC)CCCCOC(C)=O] |
| I-34 | 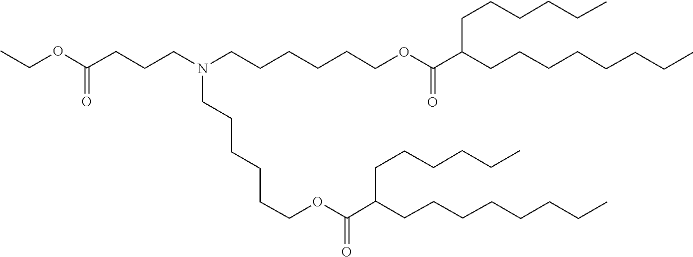 [SMILES: CCCCCCCCC(CCCCCC)C(=O)OCCCCCCN(CCCCCCOC(=O)C(CCCCCC)CCCCCCCC)CCCC(=O)OCC] |
| I-35 | 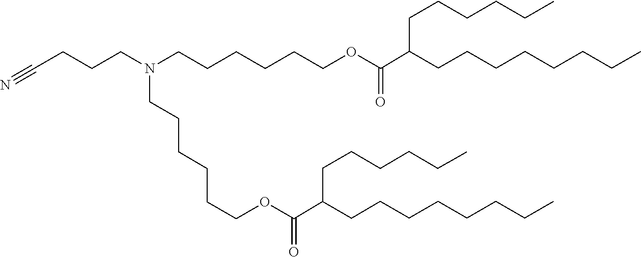 [SMILES: CCCCCCCCC(CCCCCC)C(=O)OCCCCCCN(CCCC#N)CCCCCCOC(=O)C(CCCCCC)CCCCCCCC] |
| I-36 | 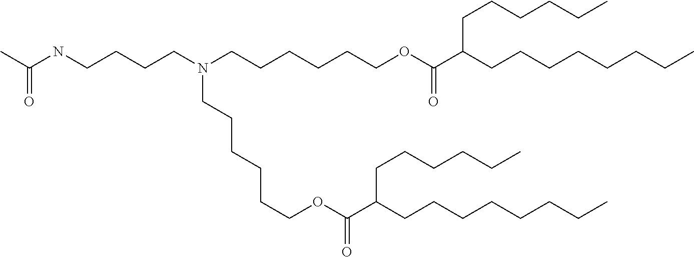 |
| I-37 | 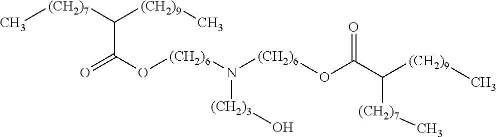 |
| I-38 | 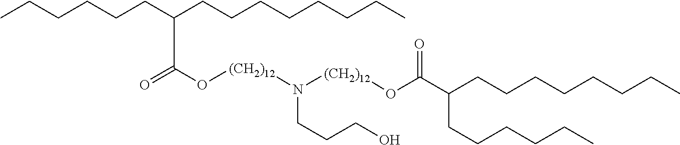 |
| I-39 | 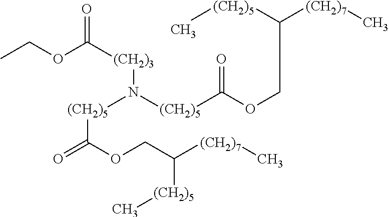 |
| I-40 | 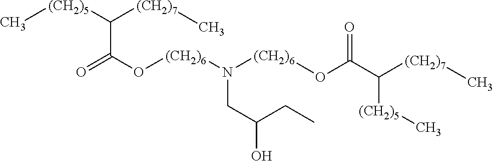 |
| I-41 | 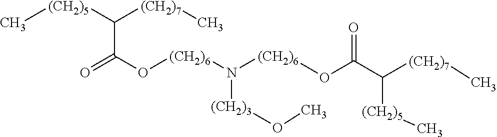 |
| I-42 | 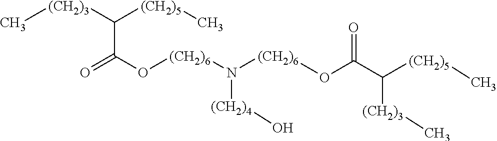 |
| I-43 | 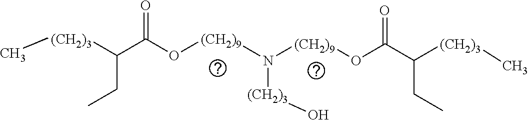 |
| I-44 | 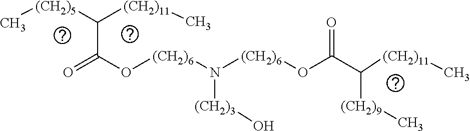 |
| I-45 | 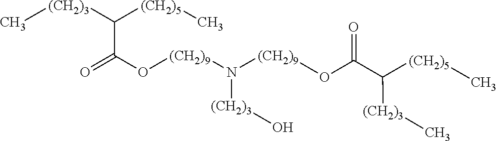 |
| I-46 | 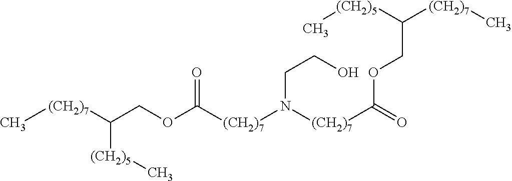 |
| I-47 | 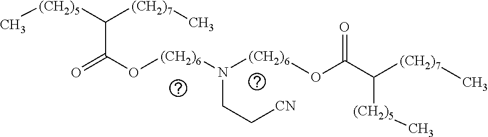 |
| I-48 | 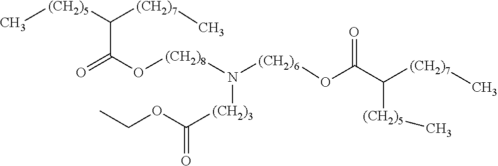 |
| I-49 | 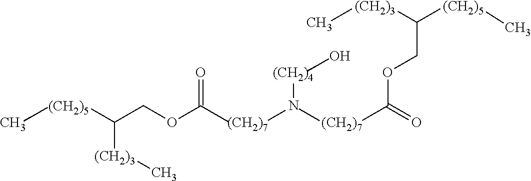 |

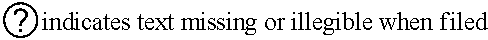

or a pharmaceutically acceptable salt thereof. In some embodiments, provided compounds are provided and/or utilized in a salt form (e.g., a pharmaceutically acceptable salt form). Reference to a compound provided herein is understood to include reference to salts thereof, unless otherwise indicated.

In certain embodiments, a cationic lipid that may be useful in accordance with the present disclosure is or comprises ((3-hydroxypropyl) azanediyl)bis(nonane-9,1-diyl)bis(2-butyloctanoate) with a chemical structure in Table B above as I-45.

In some embodiments, a cationic lipid is selected from DODAC, DOTMA, DDAB, DOTAP, DC-Chol, DMRIE, I-3, I-45, and combinations thereof.

In some embodiments, a cationic lipid is I-3. In some embodiments, a cationic lipid is I-45. In some embodiments, a cationic lipid is SM-102. In some embodiments, a cationic lipid is DODAC. In some embodiments, a cationic lipid is DOTMA. In some embodiments, a cationic lipid is DDAB. In some embodiments, a cationic lipid is DOTAP. In some embodiments, a cationic lipid is DC-Chol.

In some embodiments, lipid nanoparticles of the present disclosure comprise about 30 to about 70 mol % of a cationic lipid. In some embodiments, an lipid nanoparticle comprises about 35 to about 65 mol % of a cationic lipid. In some embodiments, an lipid nanoparticle comprises about 40 to about 60 mol % of a cationic lipid. In some embodiments, an lipid nanoparticle comprises about 41 to about 49 mol % of a cationic lipid. In some embodiments, an lipid nanoparticle comprises about 48 mol % of a cationic lipid. In some embodiments, an lipid nanoparticle comprises about 50 mol % of a cationic lipid.

Cationic lipids may be used alone or in combination with neutral lipids, e.g., cholesterol and/or neutral phospholipids, or in combination with other known lipid assembly components.

b. Helper Lipids

As described herein, lipid nanoparticles of the present disclosure comprise one or more helper lipids. In some embodiments, a lipid nanoparticle for delivery of at least one polyribonucleotide described herein comprises one or more helper lipids. A helper lipid may be a neutral lipid, a positively charged lipid, or a negatively charged lipid. In some embodiments, a helper lipid is a lipid that are useful for increasing the effectiveness of delivery of lipid-based particles such as cationic lipid-based particles to a target cell. In some embodiments, a helper lipid may be or comprise a structural lipid with its concentration chosen to optimize lipid nanoparticle particle size, stability, and/or encapsulation.

In some embodiments, a lipid nanoparticle for delivery of polyribonucleotide(s) described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. In some embodiments, a steroid is a sterol. In some embodiments, a sterol is cholesterol.

Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid nanoparticles described herein. In some embodiments, a lipid nanoparticle for delivery of polyribonucleotide(s) described herein comprises DSPC and/or cholesterol.

In some embodiments, a lipid nanoparticle described herein comprises multiple neutral lipids (e.g., two neutral lipids). It is understood that reference to “a” neutral lipid is intended to refer to lipid nanoparticles that comprise one or more neutral lipids. In some embodiments, a lipid nanoparticle described herein comprises a phospholipid and/or a steroid. In some embodiments, a lipid nanoparticle described herein comprises DSPC and/or cholesterol.

In some embodiments, a lipid nanoparticle comprises about 5 to about 15 mol % of a phospholipid. In some embodiments, a lipid nanoparticle comprises about 8 to about 12 mol % of a phospholipid. In some embodiments, a lipid nanoparticle comprises about 10 mol % of a phospholipid. In some embodiments, a lipid nanoparticle comprises about 5 to about 15 mol % of DSPC. In some embodiments, a lipid nanoparticle comprises about 8 to about 12 mol % of DSPC. In some embodiments, a lipid nanoparticle comprises about 10 mol % of DSPC.

In some embodiments, a lipid nanoparticle comprises about 30 to about 50 mol % of a steroid. In some embodiments, a lipid nanoparticle comprises about 35 to about 45 mol % of a steroid. In some embodiments, a lipid nanoparticle comprises about 38 to about 40 mol % of a steroid. In some embodiments, a lipid nanoparticle comprises about 38.5 mol % of a steroid. In some embodiments, a lipid nanoparticle comprises about 40 mol % of a steroid.

In some embodiments, a lipid nanoparticle comprises about 30 to about 50 mol % of cholesterol. In some embodiments, a lipid nanoparticle comprises about 35 to about 45 mol % of cholesterol. In some embodiments, a lipid nanoparticle comprises about 38 to about 41 mol % of cholesterol. In some embodiments, a lipid nanoparticle comprises about 38.5 mol % of cholesterol. In some embodiments, a lipid nanoparticle comprises about 40.7 mol % of cholesterol.

In some embodiments, a lipid nanoparticle comprises about 5 to about 15 mol % of phospholipid and about 30 to about 50 mol % of steroid.

In some embodiments, a lipid nanoparticle for delivery of at least one polyribonucleotide described herein comprises at least two helper lipids (e.g., ones described herein). In some such embodiments, a lipid nanoparticle for delivery of at least one polyribonucleotide described herein comprises DSPC and cholesterol.

c. Polymer-Conjugated Lipids

As described herein, lipid nanoparticles of the present disclosure comprise a polymer-conjugated lipid. In some embodiments, a lipid nanoparticle for delivery of at least one polyribonucleotide described herein comprises a polymer-conjugated lipid. A polymer-conjugated lipid is typically a molecule comprising a lipid portion and a polymer portion conjugated thereto.

In some embodiments, a polymer-conjugated lipid is a PEG-conjugated lipid. In some embodiments, a PEG-conjugated lipid is designed to sterically stabilize a lipid particle by forming a protective hydrophilic layer that shields the hydrophobic lipid layer. In some embodiments, a PEG-conjugated lipid can reduce its association with serum proteins and/or the resulting uptake by the reticuloendothelial system when such lipid particles are administered in vivo.

In some embodiments, a PEG lipid is selected from pegylated diacylglycerol (PEG-DAG) such as 1-(monomethoxy-polyethyleneglycol)-2,3-dimyristoylglycerol (PEG-DMG) (e.g., 1,2-dimyristoyl-rac-glycero-3-methoxypolyethylene glycol-2000 (PEG2000-DMG)), a pegylated phosphatidylethanoloamine (PEG-PE), a PEG succinate diacylglycerol (PEG-S-DAG) such as 4—O-(2′,3′-di(tetradecanoyloxy)propyl-1—O-(ω-methoxy(polyethoxy)ethyl) butanedioate (PEG-S-DMG), a pegylated ceramide (PEG-cer), or a PEG dialkoxypropylcarbamate such as ω-m ethoxy (polyethoxy)ethyl-N-(2,3-di(tetradecanoxy)propyl)carbamate, and 2,3-di(tetradecanoxy) propy 1-N-(ω) methoxy(polyethoxy)ethyl)carbamate.

Certain PEG-conjugated lipids (also known as PEGylated lipids) were clinically approved with safety demonstrated in clinical trials. PEG-conjugated lipids are known to affect cellular uptake, a prerequisite to endosomal localization and payload delivery. The present disclosure, among other things, provides an insight that the pharmacology of encapsulated nucleic acid can be controlled in a predictable manner by modulating the alkyl chain length of a PEG-lipid anchor. In some embodiments, the present disclosure, among other things, provides an insight that such PEG-conjugated lipids may be selected for an polyribonucleotide/lipid nanoparticle drug product formulation to provide optimum delivery of polyribonucleotides to the liver. In some embodiments, such PEG-conjugated lipids may be designed and/or selected based on reasonable solubility characteristics and/or its molecular weight to effectively perform the function of a steric barrier. For example, in some embodiments, such a PEGylated lipid does not show appreciable surfactant or permeability enhancing or disturbing effects on biological membranes. In some embodiments, PEG in such a PEG-conjugated lipid can be linked to diacyl lipid anchors with a biodegradable amide bond, thereby facilitating fast degradation and/or excretion. In some embodiments, a lipid nanoparticle comprising a PEG-conjugated lipid retain a full complement of a PEGylated lipid. In the blood compartment, such a PEGylated lipid dissociates from the particle over time, revealing a more fusogenic particle that is more readily taken up by cells, ultimately leading to release of the RNA payload.

In some embodiments, a PEG-lipid is PEG2000-DMG:

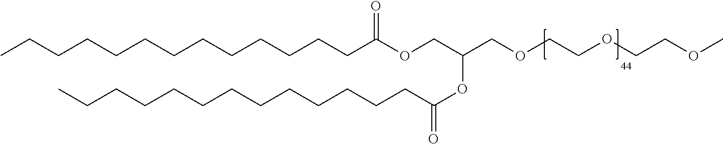

In some embodiments, a lipid nanoparticle may comprise one or more PEG-conjugated lipids or pegylated lipids as described in WO 2017/075531 and WO 2018/081480, the entire contents of each of which are incorporated herein by reference for the purposes described herein. For example, in some embodiments, a PEG-conjugated lipid that may be useful in accordance with the present disclosure can have a structure

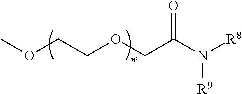

as described in WO 2017/075531, or a pharmaceutically acceptable salt, tautomer or stereoisomer thereof, wherein: R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R⁸ and R⁹ are each independently straight, saturated alkyl chains containing from 12 to 16 carbon atoms. In some embodiments, w has a mean value ranging from 43 to 53. In some embodiments, w is an integer from 40 to 50. In some embodiments, w is 45 to 47. In other embodiments, the average w is about 45. In some embodiments, a PEG-conjugated lipid is or comprises 2-[(Polyethylene glycol)-2000]-N,N-ditetradecylacetamide with a chemical structure as shown as I-3 in Table B above and below:

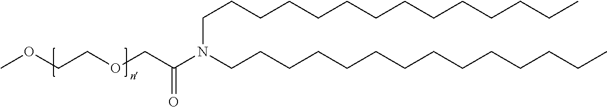

or a pharmaceutically acceptable salt thereof, where n′ is an integer from 45 to 50.

In some embodiments, a PEG-lipid is selected from PEG-DAG, PEG-PE, PEG-S-DAG, PEG2000-DMG, PEG-cer, a PEG dialkyoxypropylcarbamate, ALC-0159, and combinations thereof. In some embodiments, a PEG-lipid is ALC-0159 or PEG2000-DMG. In some embodiments, a PEG-lipid is ALC-0159. In some embodiments, a PEG-lipid is PEG2000-DMG. In some embodiments, a PEG-lipid is PEG-DAG. In some embodiments, a PEG-lipid is PEG-PE. In some embodiments, a PEG-lipid is PEG-S-DAG. In some embodiments, a PEG-lipid is PEG-cer. In some embodiments, a PEG-lipid is a PEG dialkyoxypropylcarbamate.

In some embodiments, a PEG group that is part of a PEG-lipid has, on average in a composition comprising one or more PEG-lipid molecules, a number average molecular weight (Mn) of about 2000 g/mol.

In some embodiments, a PEG-lipid is about 0.5 to about 5 mol % relative to total lipids in the lipid nanoparticle. In some embodiments, an lipid nanoparticle comprises about 1.0 to about 2.5 mol % of a PEG-lipid. In some embodiments, an lipid nanoparticle comprises about 1.5 to about 2.0 mol % of a PEG-lipid. In some embodiments, an lipid nanoparticle comprises about 1.5 to about 1.8 mol % of a PEG-lipid.

In some embodiments, a molar ratio of total cationic lipid to total polymer-conjugated lipid (e.g., PEG-conjugated lipid) is from about 100:1 to about 20:1. In some embodiments, a molar ratio of total cationic lipid to total polymer-conjugated lipid (e.g., PEG-conjugated lipid) is from about 50:1 to about 20:1. In some embodiments, a molar ratio of total cationic lipid to total polymer-conjugated lipid (e.g., PEG-conjugated lipid) is from about 40:1 to about 20:1. In some embodiments, a molar ratio of total cationic lipid to total polymer-conjugated lipid (e.g., PEG-conjugated lipid) is from about 35:1 to about 25:1.

In some embodiments, an lipid nanoparticle comprises i) about 30 to about 50 mol % of the cationic lipid; ii) about 1 to about 5 mol % of a PEG-lipid; iii) about 5 to about 15 mol % of a neutral lipid; and iv) about 30 to about 50 mol % of a steroid. In some embodiments, an lipid nanoparticle comprises: i) about 30% to about 50% by weight of ALC-0315; ii) about 1% to about 5% by weight of a ALC-0159; iii) about 5% to about 15% by weight of DSPC; and iv) about 30 to about 50 mol % of cholesterol.

In some embodiments, an lipid nanoparticle comprises: i) about 47.5 mol % of ALC-0315; ii) about 1.8 mol % of a ALC-0159; iii) about 10 mol % of DSPC; and iv) about 40.7 mol % of cholesterol.

In some embodiments, an lipid nanoparticle comprises: i) about 30 to about 50 mol % of SM-102; ii) about 1 to about 5 mol % of a PEG2000-DMG; iii) about 5 to about 15 mol % of DSPC; and iv) about 30 to about 50 mol % of a steroid. In some embodiments, an lipid nanoparticle comprises i) about 50 mol % of SM-102; ii) about 1.5 mol % of PEG2000-DMG; iii) about 10 mol % of DSPC; and iv) about 38.5 mol % of cholesterol.

### Exemplary Lipid Nanoparticle Compositions

In some embodiments, lipids that form lipid nanoparticles described herein comprise: a polymer-conjugated lipid; a cationic lipid; and at least one helper lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, about 46-48 mol %, or about 47.2-47.8 mol % of the total lipids. In certain embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 40-46 mol %, about 40-44 mol %, or about 40-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-55 mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in 45-48.5 mol % of the total lipids; the non-steroid neutral lipid is present in 9-11 mol % of the total lipids; and the steroid neutral lipid is present in about 36-44 mol % of the total lipids. In many of such embodiments, a PEG-conjugated lipid is or comprises 2-[(polyethylene glycol)-2000]-N,N-ditetradecylacetamide or a derivative thereof. In many of such embodiments, a cationic lid is or comprises ((3-hydroxypropyl) azanediyl)bis(nonane-9,1-diyl)bis(2-butyloctanoate) or a derivative thereof. In many of such embodiments, a neutral lipid comprises DSPC and cholesterol, wherein DSPC is a non-steroid neutral lipid and cholesterol is a steroid neutral lipid.

In some embodiments, lipids that form the lipid nanoparticles comprise:
(a) 2-[(polyethylene glycol)-2000]-N,N-ditetradecylacetamide at about 1-2.5 mol % of the total lipids;
(b) DPSC and cholesterol, wherein together DPSC and cholesterol are about 35-65 mol % of the total lipids; and
(c) ((3-hydroxypropyl) azanediyl)bis(nonane-9,1-diyl)bis(2-butyloctanoate) at about 35-65 mol % of the total lipids.

### Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or with ssRNs) may be monitored by appropriate techniques such as, e.g., small-angle X-ray scattering (SAXS) and/or transmission electron cryomicroscopy (CryoTEM).

In some embodiments, the viral epitope peptides and polypeptides described herein can be expressed by attenuated viruses, such as vaccinia or fowlpox. This approach involves the use of vaccinia virus as a vector to express nucleotide sequences that encode the peptide described herein. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides described herein will be apparent to those skilled in the art from the description herein.

Adjuvants are any substance whose admixture into the immunogenic composition increases or otherwise modifies the immune response to the therapeutic agent. Carriers are scaffold structures, for example a polypeptide or a polysaccharide, to which a viral epitope polypeptide or polynucleotide, is capable of being associated. Optionally, adjuvants are conjugated covalently or non-covalently to the polypeptides or polynucleotides described herein.

The ability of an adjuvant to increase the immune response to an antigen is typically manifested by a significant increase in immune-mediated reaction, or reduction in disease symptoms. For example, an increase in humoral immunity can be manifested by a significant increase in the titer of antibodies raised to the antigen, and an increase in T cell activity can be manifested in increased cell proliferation, or cellular cytotoxicity, or cytokine secretion. An adjuvant can also alter an immune response, for example, by changing a primarily humoral or T helper 2 response into a primarily cellular, or T helper 1 response.

Suitable adjuvants are known in the art (see, WO 2015/095811) and include, but are not limited to poly(I:C), poly-I and poly C, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, Lipo Vac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®. vector system, PLG microparticles, resiquimod, SRL172, virosomes and other virus-like particles, YF-17D, VEGF trap, R848, beta-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998; 186 (1): 18-27; Allison A C; Dev Biol Stand. 1998; 92:3-11) (Mosca et al. Frontiers in Bioscience, 2007; 12:4050-4060) (Gamvrellis et al. Immunol & Cell Biol. 2004; 82:506-516). Also, cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J Immunother Emphasis Tumor Immunol. 1996 (6): 414-418).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. Importantly, it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enabled the antigen doses to be reduced with comparable antibody responses to the full-dose vaccine without CpG in some experiments (Arthur M. Krieg, Nature Reviews, Drug Discovery, 5, June 2006, 471484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a component of the pharmaceutical composition described herein. Other TLR binding molecules such as RNA binding TLR 7, TLR 8 and/or TLR 9 can also be used.

Other examples of useful adjuvants include, but are not limited to, chemically modified CpGs (e.g. CpR, Idera), Poly(I:C) (e.g. polyi:C12U), non-CpG bacterial DNA or RNA, ssRNA40 for TLR8, as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which can act therapeutically and/or as an adjuvant. The amounts and concentrations of adjuvants and additives useful in the context of the present disclosure can readily be determined by the skilled artisan without undue experimentation. Additional adjuvants include colony-stimulating factors, such as Granulocyte Macrophage Colony Stimulating Factor (GM-CSF, sargramostim).

In some embodiments, an immunogenic composition according to the present disclosure can comprise more than one different adjuvants. Furthermore, present disclosure encompasses a therapeutic composition comprising any adjuvant substance including any of the above or combinations thereof. It is also contemplated that the viral epitope therapeutic can elicit or promote an immune response (e.g., a humoral or cell-mediated immune response). In some embodiments, the immunogenic composition comprises viral epitope therapeutics (e.g., peptides, polynucleotides, TCR, CAR, cells containing TCR or CAR, dendritic cell containing polypeptide, dendritic cell containing polynucleotide, antibody, etc.) and the adjuvant can be administered separately in any appropriate sequence.

A carrier can be present independently of an adjuvant. The function of a carrier can for example be to increase the molecular weight of in particular mutant in order to increase their activity or immunogenicity, to confer stability, to increase the biological activity, or to increase serum half-life. Furthermore, a carrier can aid presenting peptides to T cells. The carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. In one embodiment, the carrier comprises a human fibronectin type III domain (Koide et al. Methods Enzymol. 2012; 503:135-56). For immunization of humans, the carrier must be a physiologically acceptable carrier acceptable to humans and safe. However, tetanus toxoid and/or diptheria toxoid are suitable carriers in one embodiment of the present disclosure. Alternatively, the carrier can be dextrans for example sepharose.

In some embodiments, the polypeptides can be synthesized as multiply linked peptides as an alternative to coupling a polypeptide to a carrier to increase immunogenicity. Such molecules are also known as multiple antigenic peptides (MAPS).

In one aspect, the method presented herein comprises isolating and/or characterizing one or more virus (e.g., in some embodiments coronavirus) antigenic peptides or nucleic acids encoding characterizing one or more virus (e.g., in some embodiments coronavirus) antigenic peptides, wherein the viral (e.g., in some embodiments coronavirus) antigenic peptides are predicted to bind to one or more HLA encoded MHC class I or MHC Class II molecules expressed in a subject, wherein the subject is in need of a virus (e.g., in some embodiments coronavirus) immunotherapy such as a virus (e.g., in some embodiments coronavirus) vaccine thereof. In some embodiments, the method comprises: (a) processing amino acid information of a plurality of candidate peptide sequences using a machine learning HLA peptide presentation prediction model to generate a plurality of presentation predictions, wherein each candidate peptide sequence of the plurality of candidate peptide sequences is encoded by a genome or exome of a virus (e.g., in some embodiments coronavirus), wherein the plurality of presentation predictions comprises an HLA presentation prediction for each of the plurality of candidate viral peptide sequences, wherein each HLA presentation prediction is indicative of a likelihood that one or more proteins encoded by a class II HLA allele of a cell of the subject can present a given candidate viral peptide sequence of the plurality of candidate viral peptide sequences, wherein the machine learning HLA peptide presentation prediction model is trained using training data comprising sequence information of sequences of training peptides identified by mass spectrometry to be presented by an HLA protein expressed in training cells; and (b) identifying, based at least on the plurality of presentation predictions, a viral peptide sequence of the plurality of peptide sequences as being presented by at least one of the one or more proteins encoded by a class II HLA allele of a cell of the subject; wherein the machine learning HLA peptide presentation prediction model has a positive predictive value (PPV) of at least 0.07 according to a presentation PPV determination method.

Provided herein is a method comprising: (a) processing amino acid information of a plurality of peptide sequences of encoded by a genome or exome of a virus (e.g., in some embodiments coronavirus), using a machine learning HLA peptide binding prediction model to generate a plurality of binding predictions, wherein the plurality of binding predictions comprises an HLA binding prediction for each of the plurality of candidate peptide sequences, each binding prediction indicative of a likelihood that one or more proteins encoded by a class II HLA allele of a cell of the subject binds to a given candidate peptide sequence of the plurality of candidate peptide sequences, wherein the machine learning HLA peptide binding prediction model is trained using training data comprising sequence information of sequences of peptides identified to bind to an HLA class II protein or an HLA class II protein analog; and (b) identifying, based at least on the plurality of binding predictions, a peptide sequence of the plurality of peptide sequences that has a probability greater than a threshold binding prediction probability value of binding to at least one of the one or more proteins encoded by a class II HLA allele of a cell of the subject; wherein the machine learning HLA peptide binding prediction model has a positive predictive value (PPV) of at least 0.1 according to a binding PPV determination method.

In some embodiments, the machine learning HLA peptide presentation prediction model is trained using training data comprising sequence information of sequences of training peptides identified by mass spectrometry to be presented by an HLA protein expressed in training cells.

In some embodiments, the method comprises ranking, based on the presentation predictions, at least two peptides identified as being presented by at least one of the one or more proteins encoded by a class II HLA allele of a cell of the subject.

In some embodiments, the method comprises selecting one or more peptides of the two or more ranked peptides.

In some embodiments, the method comprises selecting one or more peptides of the plurality that were identified as being presented by at least one of the one or more proteins encoded by a class II HLA allele of a cell of the subject.

In some embodiments, the method comprises selecting one or more peptides of two or more peptides ranked based on the presentation predictions.

In some embodiments, the machine learning HLA peptide presentation prediction model has a positive predictive value (PPV) of at least 0.07 when amino acid information of a plurality of test peptide sequences are processed to generate a plurality of test presentation predictions, each test presentation prediction indicative of a likelihood that the one or more proteins encoded by a class II HLA allele of a cell of the subject can present a given test peptide sequence of the plurality of test peptide sequences, wherein the plurality of test peptide sequences comprises at least 500 test peptide sequences comprising (i) at least one hit peptide sequence identified by mass spectrometry to be presented by an HLA protein expressed in cells and (ii) at least 499 decoy peptide sequences contained within a protein encoded by a genome of an organism, wherein the organism and the subject are the same species, wherein the plurality of test peptide sequences comprises a ratio of 1:499 of the at least one hit peptide sequence to the at least 499 decoy peptide sequences and a top percentage of the plurality of test peptide sequences are predicted to be presented by the HLA protein expressed in cells by the machine learning HLA peptide presentation prediction model.

In some embodiments, the machine learning HLA peptide presentation prediction model has a positive predictive value (PPV) of at least 0.1 when amino acid information of a plurality of test peptide sequences are processed to generate a plurality of test binding predictions, each test binding prediction indicative of a likelihood that the one or more proteins encoded by a class II HLA allele of a cell of the subject binds to a given test peptide sequence of the plurality of test peptide sequences, wherein the plurality of test peptide sequences comprises at least 20 test peptide sequences comprising (i) at least one hit peptide sequence identified by mass spectrometry to be presented by an HLA protein expressed in cells and (ii) at least 19 decoy peptide sequences contained within a protein comprising at least one peptide sequence identified by mass spectrometry to be presented by an HLA protein expressed in cells, such as a single HLA protein expressed in cells (e.g., mono-allelic cells), wherein the plurality of test peptide sequences comprises a ratio of 1:19 of the at least one hit peptide sequence to the at least 19 decoy peptide sequences and a top percentage of the plurality of test peptide sequences are predicted to bind to the HLA protein expressed in cells by the machine learning HLA peptide presentation prediction model.

In some embodiments, no amino acid sequence overlap exist among the at least one hit peptide sequence and the decoy peptide sequences.

### Combinations of CTL Peptides and HTL Peptides

Immunogenic or vaccine compositions comprising the viral epitope polypeptides and polynucleotides described herein, or analogs thereof, which have immunostimulatory activity can be modified to provide desired attributes, such as improved serum half-life, or to enhance immunogenicity.

For instance, the ability of the viral epitope peptides to induce CTL activity can be enhanced by linking the peptide to a sequence which contains at least one epitope that is capable of inducing a T helper cell response. In one embodiment, CTL epitope/HTL epitope conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the CTL peptide can be linked to the T helper peptide without a spacer.

Although the CTL peptide epitope can be linked directly to the T helper peptide epitope, CTL epitope/HTL epitope conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. The CTL peptide epitope can be linked to the T helper peptide epitope either directly or via a spacer either at the amino or carboxy terminus of the CTL peptide. The amino terminus of either the immunogenic peptide or the T helper peptide can be acylated.

HTL peptide epitopes can also be modified to alter their biological properties. For example, peptides comprising HTL epitopes can contain D-amino acids to increase their resistance to proteases and thus extend their serum half-life. Also, the epitope peptides can be conjugated to other molecules such as lipids, proteins or sugars, or any other synthetic compounds, to increase their biological activity. For example, the T helper peptide can be conjugated to one or more palmitic acid chains at either the amino or carboxyl termini.

In certain embodiments, the T helper peptide is one that is recognized by T helper cells present in the majority of the population. This can be accomplished by selecting amino acid sequences that bind to many, most, or all of the HLA class II molecules. These are known as “loosely HLA-restricted” or “promiscuous” T helper sequences. Examples of amino acid sequences that are promiscuous include sequences from antigens such as tetanus toxoid at positions 830-843 (QYIKANSKFIGITE), Plasmodium falciparum CS protein at positions 378-398 (DIEKKIAKMEKASSVENVVNS), and Streptococcus 18 kD protein at positions 116 (GAVDSILGGVATYGAA). Other examples include peptides bearing a DR 1-4-7 supermotif, or either of the DR3 motifs.

Alternatively, it is possible to prepare synthetic peptides capable of stimulating T helper lymphocytes, in a loosely HLA-restricted fashion, using amino acid sequences not found in nature (see, e.g., PCT publication WO 95/07707). These synthetic compounds called Pan-DR-binding epitopes (e.g., PADRE, Epimmune, Inc., San Diego, CA) are designed to bind most HLA-DR (human HLA class II) molecules. For instance, a pan-DR-binding epitope peptide having the formula: aKXVWANTLKAAa, where “X” is either cyclohexyl alanine, phenylalanine, or tyrosine, and a is either D-alanine or L-alanine, has been found to bind to most HLA-DR alleles, and to stimulate the response of T helper lymphocytes from most individuals, regardless of their HLA type. An alternative of a pan-DR binding epitope comprises all “L” natural amino acids and can be provided in the form of nucleic acids that encode the epitope.

In some embodiments it can be desirable to include in a viral epitope therapeutic (e.g., peptides, polynucleotides, TCR, CAR, cells containing TCR or CAR, dendritic cell containing polypeptide, dendritic cell containing polynucleotide, antibody, etc.) in pharmaceutical compositions (e.g., immunogenic compositions) at least one component of which primes cytotoxic T lymphocytes. Lipids have been identified as agents capable of priming CTL in vivo against viral antigens. For example, palmitic acid residues can be attached to the c- and a-amino groups of a lysine residue and then linked, e.g., via one or more linking residues such as Gly, Gly-Gly-, Ser, Ser-Ser, or the like, to an immunogenic viral epitope peptide. The lipidated peptide can then be administered either directly in a micelle or particle, incorporated into a liposome, or emulsified in an adjuvant. In one embodiment, a particularly effective immunogenic construct comprises palmitic acid attached to c- and a-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P3CSS) can be used to prime virus specific CTL when covalently attached to an appropriate peptide. (See, e.g., Deres, et al., Nature 342:561, 1989). Viral epitope peptides described herein can be coupled to P3CSS, for example, and the lipopeptide administered to an individual to specifically prime a CTL response to the target antigen. Moreover, because the induction of neutralizing antibodies can also be primed with P3CSS-conjugated epitopes, two such compositions can be combined to more effectively elicit both humoral and cell-mediated responses to infection.

As noted herein, additional amino acids can be added to the termini of a viral epitope peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. However, it is to be noted that modification at the carboxyl terminus of a T cell epitope can, in some cases, alter binding characteristics of the peptide. In addition, the peptide or oligopeptide sequences can differ from the natural sequence by being modified by terminal-NH2 acylation, e.g., by alkanoyl (C1-C20) or thioglycolyl acetylation, terminal-carboxyl amidation, e.g., ammonia, methylamine, etc. In some instances, these modifications can provide sites for linking to a support or other molecule.

An embodiment of an immunogenic composition described herein comprises ex vivo administration of a cocktail of epitope-bearing viral epitope polypeptide or polynucleotides to PBMC, or DC therefrom, from the patient's blood. A pharmaceutical to facilitate harvesting of dendritic cells (DCs) can be used, including GM-CSF, IL-4, IL-6, IL-1b, and TNFα. After pulsing the DCs with peptides or polynucleotides encoding the peptides, and prior to reinfusion into patients, the DC are washed to remove unbound peptides. In this embodiment, a vaccine or immunogenic composition comprises peptide-pulsed DCs which present the pulsed peptide epitopes complexed with HLA molecules on their surfaces. The composition is then administered to the patient. In other embodiments, such pulsed DCs are used to stimulate T cells suitable for use in T cell therapy.

### Multi-Epitope Immunogenic Compositions

A number of different approaches are available which allow simultaneous delivery of multiple epitopes. Nucleic acids encoding the viral epitope peptides described herein are a particularly useful embodiment of the present disclosure. In one embodiment, the nucleic acid is RNA. In one embodiment, the nucleic acid is mRNA. In some embodiments, minigene constructs encoding a viral epitope peptide comprising one or multiple epitopes described herein may be used to administer nucleic acids encoding the viral epitope peptides described herein. In some embodiments, a RNA construct (e.g., mRNA construct) encoding a viral epitope peptide comprising one or multiple epitopes described herein is administered.

Exemplary use of multi-epitope minigenes is described An, L. and Whitton, J. L., J. Virol. 71:2292, 1997; Thomson, S. A. et al., J. Immunol. 157:822, 1996; Whitton, J. L. et al., J. Virol 67:348, 1993; Hanke, R. et al., Vaccine 16:426, 1998. For example, a multi-epitope DNA plasmid encoding super motif- and/or motif-bearing antigen peptides, a universal helper T cell epitope (or multiple viral antigen HTL epitopes), and an endoplasmic reticulum-translocating signal sequence can be engineered.

The immunogenicity of a multi-epitopic minigene can be tested in transgenic mice to evaluate the magnitude of immune response induced against the epitopes tested. Further, the immunogenicity of DNA-encoded epitopes in vivo can be correlated with the in vitro responses of specific CTL lines against target cells transfected with the DNA plasmid. Thus, these experiments can show that the minigene serves to both: 1). generate a cell mediated and/or humoral response and 2). that the induced immune cells recognized cells expressing the encoded epitopes.

For example, to create a DNA sequence encoding the selected viral epitope (minigene) for expression in human cells, the amino acid sequences of the epitopes can be reverse translated. A human codon usage table can be used to guide the codon choice for each amino acid. These viral epitope-encoding DNA sequences can be directly adjoined, so that when translated, a continuous polypeptide sequence is created. To optimize expression and/or immunogenicity, additional elements can be incorporated into the minigene design. Examples of amino acid sequences that can be reverse translated and included in the minigene sequence include: HLA class I epitopes, HLA class II epitopes, a ubiquitination signal sequence, and/or an endoplasmic reticulum targeting signal. In addition, HLA presentation of CTL and HTL epitopes can be improved by including synthetic (e.g. poly-alanine) or naturally-occurring flanking sequences adjacent to the CTL or HTL epitopes; these larger peptides comprising the epitope(s) are within the scope of the present disclosure.

The minigene sequence can be converted to DNA by assembling oligonucleotides that encode the plus and minus strands of the minigene. Overlapping oligonucleotides (30-100 bases long) can be synthesized, phosphorylated, purified and annealed under appropriate conditions using well known techniques. The ends of the oligonucleotides can be joined, for example, using T4 DNA ligase. This synthetic minigene, encoding the epitope polypeptide, can then be cloned into a desired expression vector.

Standard regulatory sequences well known to those of skill in the art can be included in the vector to ensure expression in the target cells. For example, a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences.

Additional vector modifications can be used to optimize minigene expression and immunogenicity. In some cases, introns are utilized for efficient gene expression, and one or more synthetic or naturally-occurring introns could be incorporated into the transcribed region of the minigene. The inclusion of mRNA stabilization sequences and sequences for replication in mammalian cells can also be considered for increasing minigene expression.

Once an expression vector is selected, the minigene can be cloned into the polylinker region downstream of the promoter. This plasmid is transformed into an appropriate E. coli strain, and DNA is prepared using standard techniques. The orientation and DNA sequence of the minigene, as well as all other elements included in the vector, can be confirmed using restriction mapping and DNA sequence analysis. Bacterial cells harboring the correct plasmid can be stored as a master cell bank and a working cell bank.

In addition, immunomodulatory sequences appear to play a role in the immunogenicity of DNA vaccines. These sequences can be included in the vector, outside the minigene coding sequence, if desired to enhance immunogenicity. In one embodiment, the sequences are immunostimulatory. In another embodiment, the sequences are ISSs or CpGs.

In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins. Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-(3) can be beneficial in certain diseases.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well-known techniques. Plasmid DNA can be purified using standard bioseparation technologies such as solid phase anion-exchange resins supplied by QIAGEN, Inc. (Valencia, California). If required, supercoiled DNA can be from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. To maximize the immunotherapeutic effects of minigene DNA vaccines, an alternative method for formulating purified plasmid DNA can be used. A variety of methods have been described, and new techniques can become available. Cationic lipids can also be used in the formulation (see, e.g., as described by WO 93/24640; Mannino & Gould-Fogerite, BioTechniques 6 (7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

In another embodiment, the nucleic acid is introduced into cells by use of high-speed cell deformation. During high-speed deformation, cells are squeezed such that temporary disruptions occur in the cell membrane, thus allowing the nucleic acid to enter the cell. Alternatively, protein can be produced from expression vectors—in a bacterial expression vector, for example, and the proteins can then be delivered to the cell.

Target cell sensitization can be used as a functional assay for expression and HLA class I presentation of minigene-encoded CTL epitopes. For example, the plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (51-Cr) labeled and used as target cells for epitope-specific CTL lines; cytolysis, detected by 51Cr release, indicates both production of, and HLA presentation of, minigene-encoded CTL epitopes. Expression of HTL epitopes can be evaluated in an analogous manner using assays to assess HTL activity.

In vivo immunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (IP) for lipid-complexed DNA). An exemplary protocol is twenty-one days after immunization, splenocytes are harvested and restimulated for 1 week in the presence of peptides encoding each epitope being tested. Thereafter, for CTL effector cells, assays are conducted for cytolysis of peptide-loaded, 51Cr-labeled target cells using standard techniques. Lysis of target cells that were sensitized by HLA loaded with peptide epitopes, corresponding to minigene-encoded epitopes, demonstrates DNA vaccine function for in vivo induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment, DNA can be adhered to particles, such as gold particles.

### Cells

In one aspect, the present disclosure also provides cells expressing a viral epitope-recognizing receptor that activates an immunoresponsive cell (e.g., T cell receptor (TCR) or chimeric antigen receptor (CAR)), and methods of using such cells for the treatment of a disease that requires an enhanced immune response.

Such cells include genetically modified immunoresponsive cells (e.g., T cells, Natural Killer (NK) cells, cytotoxic T lymphocytes (CTL) cells, helper T lymphocyte (HTL) cells) expressing an antigen-recognizing receptor (e.g., TCR or CAR) that binds one of the viral epitope peptides described herein, and methods of use therefore for the treatment of neoplasia and other pathologies where an increase in an antigen-specific immune response is desired. T cell activation is mediated by a TCR or a CAR targeted to an antigen.

The present disclosure provides cells expressing a combination of an antigen-recognizing receptor that activates an immunoresponsive cell (e.g., TCR, CAR) and a chimeric co-stimulating receptor (CCR), and methods of using such cells for the treatment of a disease that requires an enhanced immune response. In one embodiment, viral antigen-specific T cells, NK cells, CTL cells or other immunoresponsive cells are used as shuttles for the selective enrichment of one or more co-stimulatory ligands for the treatment or prevention of neoplasia. Such cells are administered to a human subject in need thereof for the treatment or prevention of a particular viral infection.

In one embodiment, the viral antigen-specific human lymphocytes that can be used in the methods of the present disclosure include, without limitation, peripheral donor lymphocytes genetically modified to express chimeric antigen receptors (CARs) (Sadelain, M., et al. 2003 Nat Rev Cancer 3:35-45), peripheral donor lymphocytes genetically modified to express a full-length viral antigen-recognizing T cell receptor complex comprising the a and p heterodimer (Morgan, R. A., et al. 2006 Science 314:126-129), and selectively in vitro-expanded antigen-specific peripheral blood leukocytes employing artificial antigen-presenting cells (AAPCs) or pulsed dendritic cells (Dupont, J., et al. 2005 Cancer Res 65:5417-5427; Papanicolaou, G. A., et al. 2003 Blood 102:2498-2505). The T cells may be autologous, allogeneic, or derived in vitro from engineered progenitor or stem cells.

### Co-Stimulatory Ligands

In one embodiment, the cells of the present disclosure are provided with at least one co-stimulatory ligand which is a non-antigen specific signal important for full activation of an immune cell. Co-stimulatory ligands include, without limitation, tumor necrosis factor (TNF) ligands, cytokines (such as IL-2, IL-12, IL-15 or IL21), and immunoglobulin (Ig) superfamily ligands. Tumor necrosis factor (TNF) is a cytokine involved in systemic inflammation and stimulates the acute phase reaction. Its primary role is in the regulation of immune cells. Tumor necrosis factor (TNF) ligands share a number of common features.

The majority of the ligands are synthesized as type II transmembrane proteins containing a short cytoplasmic segment and a relatively long extracellular region. TNF ligands include, without limitation, nerve growth factor (NGF), CD40L (CD40L)/CD154, CD137L/4-1BBL, tumor necrosis factor alpha (TNFα), CD134L/OX4OL/CD252, CD27L/CD70, Fas ligand (FasL), CD3OL/CD153, tumor necrosis factor f3 (TNF (3)/lymphotoxin-alpha (LTa), lymphotoxin-beta (ur (3), CD257/B cell-activating factor (BAFF)/Blys/THANK/Ta11-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins, they possess an immunoglobulin domain (fold). Immunoglobulin superfamily ligands include, without limitation, CD80 and CD86, both ligands for CD28.

Compositions comprising genetically modified immunoresponsive cells of the present disclosure can be provided systemically or directly to a subject for the treatment of a neoplasia. In one embodiment, cells of the present disclosure are directly injected into an organ of interest. Alternatively, compositions comprising genetically modified immunoresponsive cells are provided indirectly to the organ of interest, for example, by administration into the circulatory system. Expansion and differentiation agents can be provided prior to, during or after administration of the cells to increase production of T cells, NK cells, or CTL cells in vitro or in vivo.

The modified cells can be administered in any physiologically acceptable vehicle, normally intravascularly, although they may also be introduced into bone or other convenient site where the cells may find an appropriate site for regeneration and differentiation (e.g., thymus). The modified cells can be autologous or allogeneic. Genetically modified immunoresponsive cells of the present disclosure can comprise a purified population of cells. Those skilled in the art can readily determine the percentage of genetically modified immunoresponsive cells in a population using various well-known methods, such as fluorescence activated cell sorting (FACS). Dosages can be readily adjusted by those skilled in the art (e.g., a decrease in purity may require an increase in dosage). The cells can be introduced by injection, catheter, or the like. If desired, factors can also be included, including, but not limited to, interleukins, e.g. IL-2, IL-3, IL-6, and IL-11, as well as the other interleukins, the colony stimulating factors, such as G-, M- and GM-CSF, interferons, e.g. interferon gamma and erythropoietin.

Compositions of the present disclosure include pharmaceutical compositions comprising genetically modified immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells of the present disclosure or their progeny (e.g., in vivo, ex vivo or in vitro derived) can be administered via localized injection, including catheter administration, systemic injection, localized injection, intravenous injection, or parenteral administration. When administering a therapeutic composition of the present disclosure (e.g., a pharmaceutical composition containing a genetically modified immunoresponsive cell), it will generally be formulated in a unit dosage injectable form (solution, suspension, emulsion).

### Methods of Use and Pharmaceutical Compositions

The viral epitope therapeutics (e.g., peptides, polynucleotides, TCR, CAR, cells containing TCR or CAR, dendritic cell containing polypeptide, dendritic cell containing polynucleotide, antibody, etc.) described herein are useful in a variety of applications including, but not limited to, therapeutic treatment methods, such as the treatment or prevention of a viral infection. In some embodiments, the therapeutic treatment methods comprise immunotherapy. In certain embodiments, a viral epitope peptide is useful for activating, promoting, increasing, and/or enhancing an immune response or redirecting an existing immune response to a new target. The methods of use can be in vitro, ex vivo, or in vivo methods.

In some aspects, the present disclosure provides methods for activating an immune response in a subject using a viral epitope therapeutic described herein. In some embodiments, the present disclosure provides methods for promoting an immune response in a subject using a viral epitope therapeutic described herein. In some embodiments, the present disclosure provides methods for increasing an immune response in a subject using a viral epitope peptide described herein. In some embodiments, the present disclosure provides methods for enhancing an immune response using a viral epitope peptide. In some embodiments, the activating, promoting, increasing, and/or enhancing of an immune response comprises increasing cell-mediated immunity. In some embodiments, the activating, promoting, increasing, and/or enhancing of an immune response comprises increasing T cell activity or humoral immunity. In some embodiments, the activating, promoting, increasing, and/or enhancing of an immune response comprises increasing CTL or HTL activity. In some embodiments, the activating, promoting, increasing, and/or enhancing of an immune response comprises increasing NK cell activity. In some embodiments, the activating, promoting, increasing, and/or enhancing of an immune response comprises increasing T cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of Tregs. In some embodiments, the immune response is a result of antigenic stimulation.

In some embodiments, the present disclosure provides methods of activating, promoting, increasing, and/or enhancing of an immune response using a viral epitope therapeutic described herein. In some embodiments, a method comprises administering to a subject in need thereof a therapeutically effective amount of a viral epitope therapeutic that delivers a viral epitope polypeptide or polynucleotide to a cell. In some embodiments, a method comprises administering to a subject in need thereof a therapeutically effective amount of a viral epitope that is internalized by a cell, and the viral epitope peptide is processed by the cell. In some embodiments, a method comprises administering to a subject in need thereof a therapeutically effective amount of a viral epitope polypeptide that is internalized by a cell, and an antigenic peptide is presented on the surface of the cell. In some embodiments, a method comprises administering to a subject in need thereof a therapeutically effective amount of a viral epitope polypeptide that is internalized by the cell, is processed by the cell, and an antigenic peptide is presented on the surface of the cell.

In some embodiments, a method comprises administering to a subject in need thereof a therapeutically effective amount of a viral epitope polypeptide or polynucleotide described herein that delivers an exogenous polypeptide comprising at least one antigenic peptide to a cell, wherein the antigenic peptide is presented on the surface of the cell. In some embodiments, the antigenic peptide is presented on the surface of the cell in complex with a MHC class I molecule. In some embodiments, the antigenic peptide is presented on the surface of the cell in complex with a MHC class II molecule.

In some embodiments, a method comprises contacting a cell with a viral epitope polypeptide or polynucleotide described herein that delivers an exogenous polypeptide comprising at least one antigenic peptide to the cell, wherein the antigenic peptide is presented on the surface of the cell. In some embodiments, the antigenic peptide is presented on the surface of the cell in complex with a MHC class I molecule. In some embodiments, the antigenic peptide is presented on the surface of the cell in complex with a MHC class II molecule.

In some embodiments, a method comprises administering to a subject in need thereof a therapeutically effective amount of a viral epitope polypeptide or polynucleotide described herein that delivers an exogenous polypeptide comprising at least one antigenic peptide to a cell, wherein the antigenic peptide is presented on the surface of the cell, and an immune response against the cell is induced. In some embodiments, the immune response against the cell is increased. In some embodiments, the viral epitope polypeptide or polynucleotide delivers an exogenous polypeptide comprising at least one antigenic peptide to a cell, wherein the antigenic peptide is presented on the surface of the cell.

In some embodiments, a method comprises administering to a subject in need thereof a therapeutically effective amount of a viral epitope polypeptide or polynucleotide described herein that delivers an exogenous polypeptide comprising at least one antigenic peptide to a cell, wherein the antigenic peptide is presented on the surface of the cell, and T cell killing directed against the cell is induced. In some embodiments, T cell killing directed against the cell is enhanced. In some embodiments, T cell killing directed against the cell is increased.

In some embodiments, a method of increasing an immune response in a subject comprises administering to the subject a therapeutically effective amount of a viral epitope therapeutic described herein, wherein the agent is an antibody that specifically binds the viral epitope described herein. In some embodiments, a method of increasing an immune response in a subject comprises administering to the subject a therapeutically effective amount of the antibody.

The present disclosure provides methods of inducing or promoting or enhancing an immune response to a virus. In some embodiments, a method of inducing or promoting or enhancing an immune response to a virus comprises administering to a subject a therapeutically effective amount of a viral epitope therapeutic described herein. In some embodiments, the immune response is against a virus. In preferred embodiments, the immune response is against a coronavirus. In preferred embodiments, the immune response is against a COVID19. In some embodiments, the virus is selected from the group consisting of: measles virus, varicella-zoster virus (VZV); chickenpox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the virus is varicella-zoster virus. In some embodiments, the virus is cytomegalovirus. In some embodiments, the virus is measles virus. In some embodiments, the immune response has been acquired after a natural viral infection. In some embodiments, the immune response has been acquired after vaccination against a virus. In some embodiments, the immune response is a cell-mediated response. In some embodiments, the existing immune response comprises cytotoxic T cells (CTLs) or HTLs.

In some embodiments, a method of inducing or promoting or enhancing an immune response to a virus in a subject comprises administering a fusion protein comprising (i) an antibody that specifically binds a viral epitope and (ii) at least one viral epitope peptide described herein, wherein (a) the fusion protein is internalized by a cell after binding to the viral antigen; (b) the viral epitope peptide is processed and presented on the surface of the cell associated with a MHC class I molecule; and (c) the viral epitope peptide/MHC Class I complex is recognized by cytotoxic T cells. In some embodiments, the cytotoxic T cells are memory T cells. In some embodiments, the memory T cells are the result of a vaccination with the viral epitope peptide.

The present disclosure provides methods of increasing the immunogenicity of a virus. In some embodiments, a method of increasing the immunogenicity of a virus comprises contacting virally infected cells with an effective amount of a viral epitope therapeutic described herein. In some embodiments, a method of increasing the immunogenicity of a virus comprises administering to a subject a therapeutically effective amount of a viral epitope therapeutic described herein. In certain embodiments, the subject is a human.

In some embodiments, a method can comprise treating or preventing cancer in a subject in need thereof by administering to a subject a therapeutically effective amount of a viral epitope therapeutic described herein. In some embodiments, the cancer is a liquid cancer, such as a lymphoma or leukemia. In some embodiments, the cancer is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

The present disclosure further provides methods for treating or preventing a viral infection in a subject comprising administering to the subject a therapeutically effective amount of a viral epitope therapeutic described herein.

In some embodiments, a method of treating or preventing a viral infection comprises redirecting an existing immune response to a new target, the method comprising administering to a subject a therapeutically effective amount of viral epitope therapeutic, wherein the existing immune response is against an antigenic peptide delivered to a cell or a cell infected with a virus by the viral epitope peptide.

The present disclosure provides for methods of treating or preventing a viral infection comprising administering to a subject a therapeutically effective amount of a viral epitope therapeutic described herein (e.g., a subject in need of treatment). In certain embodiments, the subject is a human. In certain embodiments, the subject has a coronavirus infection or is at risk of a coronavirus infection.

In certain embodiments, in addition to administering a viral epitope therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

In some embodiments, the viral epitope therapeutic can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF TNF-α, VEGF, P1GF, gamma-IFN, IL-1, IL-2, IL-3, IL-4, IL-5, IL-6, IL-7, IL-12, IL-15, and IL-18.

In certain embodiments, treatment involves the administration of a viral epitope therapeutic described herein in combination with an additional therapy. In certain embodiments, the additional therapy is or comprises a therapy for another virus, for example influenza. In certain embodiments, the additional therapy is or comprises one or more vaccines (e.g., vaccines against one or more infectious disease). In certain embodiments, a viral epitope therapeutic described herein is administered in combination with a therapy (e.g., in some embodiments a vaccine) for SARS-CoV-2 infection. In certain embodiments, a viral epitope therapeutic described herein is administered in combination with a therapy (e.g., in some embodiments a vaccine) for a non-SARS-CoV-2 pathogen (e.g., a virus) infection, for example, a non-SARS-CoV-2 respiratory disease. Non-limiting examples of non-SARS-CoV-2 respiratory diseases include coronaviruses (e.g., a non-SARS-CoV-2 coronavirus), an Influenza virus, a Pneumoviridae virus, a Paramyxoviridae virus (e.g., a Respiratory syncytial virus (RSV) or a Metapneumovirus), and combinations thereof. In some embodiments, the Metapneumovirus is a human metapneumovirus (hMPV). In some embodiments, the Paramyxoviridae virus is a Parainfluenza virus or a Henipavirus. In some embodiments the parainfluenzavirus is PIV3. In some embodiments, the non-SAR-CoV-2 coronavirus is a betacoronavirus (e.g., SARS-CoV-1). In come embodiments the non-SARS-CoV-2 coronavirus is a Merbecovirus (e.g., a MERS-CoV virus).

In some embodiments, a viral epitope therapauetic described herein is administered in combination with a therapy (e.g., in some embodiments a vaccine) for influenza.

In some embodiments, a viral epitope therapeutic described herein is administered in combination with an RSV vaccine (e.g., an RSV A or RSV B vaccine). In some embodiments, the RSV vaccine comprises an RSV fusion protein (F), an RSV attachment protein (G), an RSV small hydrophobic protein (SH), an RSV matrix protein (M), an RSV nucleoprotein (N), an RSV M2-1 protein, an RSV Large polymerase (L), and/or an RSV phosphoprotein (P), or an immunogenic fragment of immunogenic variant thereof, or a nucleic acid (e.g., RNA), encoding any one of the same. In some embodiments, a therapeutic described herein is co-administered with an influenza vaccine. In some embodiments, the influenza vaccine is an alphainfluenza virus, a betainfluenza virus, a gammainfluenza virus or a deltainfluenza virus vaccine. In some embodiments the vaccine is an Influenza A virus, an Influenza B virus, an Influenza C virus, or an Influenza D virus vaccine. In some embodiments, the influenza A virus vaccine comprises a hemagglutinin selected from H1, H2, H3, H4, H5, H6, H7, H8, H9, H10, H11, H12, H13, H14, H15, H16, H17, and H18, or an immunogenic fragment or variant of the same, or a nucleic acid (e.g., RNA) encoding any one of the same. In some embodiments the influenza A vaccine comprises or encodes a neuraminidase (NA) selected from N1, N2, N3, N4, N5, N6, N7, N8, N9, N10, and N11, or an immunogenic fragment or variant of the same, or a nucleic acid (e.g., RNA) encoding any one of the same. In some embodiments, the influenza vaccine comprises at least one Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or variant thereof, or a nucleic acid (e.g., RNA) encoding any of one of the same.

Exemplary therapies for viruses also include but are not limited to oseltamivir, oseltamivir phosphate (available as a generic version or under the trade name Tamiflu®), zanamivir (trade name Relenza®), peramivir (trade name Rapivab®), baloxavir marboxil (trade name Xofluza®), amantadine, moroxydine, rimantadine, umifenovir (trade name Arbidol®) and zanamivir (trade name Relenza®).

Treatment with an agent can occur prior to, concurrently with, or subsequent to administration of an additional therapy. Dosing schedules for such additional therapies can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a viral epitope therapeutic described herein and at least one additional therapeutic agent can be administered in any order or concurrently. In some embodiments, the agent will be administered to patients that have previously undergone treatment with a second therapeutic agent. In certain other embodiments, the viral epitope therapeutic and a second therapeutic agent will be administered substantially simultaneously or concurrently. For example, a subject can be given an agent while undergoing a course of treatment with a second therapeutic agent (e.g., chemotherapy). In certain embodiments, a viral epitope therapeutic will be administered within 1 year of the treatment with a second therapeutic agent. It will further be appreciated that the two (or more) agents or treatments can be administered to the subject within a matter of hours or minutes (i.e., substantially simultaneously).

For the treatment of a disease, the appropriate dosage of a viral epitope therapeutic described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. The viral epitope therapeutic can be administered one time or over a series of treatments lasting from several days to several months, or until a cure is effected or a diminution of the disease state is achieved. Optimal dosing schedules can be calculated from measurements of drug accumulation in the body of the patient and will vary depending on the relative potency of an individual agent. The administering physician can determine optimum dosages, dosing methodologies, and repetition rates.

In some embodiments, a viral epitope therapeutic can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose. Or a dosing regimen can comprise administering an initial loading dose, followed by maintenance doses of, for example one-half of the initial dose every other week. Or a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, therapy must be discontinued, and other agents can be tried. However, many agents in the same therapeutic class display similar side effects and/or toxicities, meaning that the patient either has to stop therapy, or if possible, suffer from the unpleasant side effects associated with the therapeutic agent.

In some embodiments, the dosing schedule can be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present disclosure provides methods of administering to a subject a viral epitope therapeutic described herein comprising using an intermittent dosing strategy for administering one or more agents, which can reduce side effects and/or toxicities associated with administration of an agent, chemotherapeutic agent, etc. In some embodiments, a method for treating or preventing a viral infection in a human subject comprises administering to the subject a therapeutically effective dose of a viral epitope therapeutic in combination with a therapeutically effective dose of another therapeutic agent, such as an anti-viral agent, wherein one or both of the agents are administered according to an intermittent dosing strategy. In some embodiments, a method for treating or preventing a viral infection in a human subject comprises administering to the subject a therapeutically effective dose of a viral epitope therapeutic in combination with a therapeutically effective dose of a second viral epitope therapeutic, wherein one or both of the agents are administered according to an intermittent dosing strategy. In some embodiments, the intermittent dosing strategy comprises administering an initial dose of a viral epitope therapeutic to the subject, and administering subsequent doses of the agent about once every 2 weeks. In some embodiments, the intermittent dosing strategy comprises administering an initial dose of a viral epitope therapeutic to the subject, and administering subsequent doses of the agent about once every 3 weeks. In some embodiments, the intermittent dosing strategy comprises administering an initial dose of a viral epitope therapeutic to the subject, and administering subsequent doses of the agent about once every 4 weeks. In some embodiments, the agent is administered using an intermittent dosing strategy and the additional therapeutic agent is administered weekly.

The present disclosure provides compositions comprising the viral epitope therapeutic described herein. The present disclosure also provides pharmaceutical compositions comprising a viral epitope therapeutic described herein and a pharmaceutically acceptable vehicle. In some embodiments, the pharmaceutical compositions find use in immunotherapy. In some embodiments, the compositions find use in inhibiting viral replication. In some embodiments, the pharmaceutical compositions find use in inhibiting viral replication in a subject (e.g., a human patient).

Formulations are prepared for storage and use by combining an antigen therapeutic of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition. Exemplary formulations are listed in WO 2015/095811.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London.). In one embodiment, the vehicle is 5% dextrose in water.

The pharmaceutical compositions described herein can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories

The viral epitope peptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include a viral epitope therapeutic described herein complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the viral epitope peptides described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, nondegradable ethylene-vinyl acetate, degradable lactic acid-glycolic acid copolymers such as the LUPRON DEPOTTM (injectable microspheres composed of lactic acid-glycolic acid copolymer and leuprolide acetate), sucrose acetate isobutyrate, and poly-D-(−)-3-hydroxybutyric acid.

### String Construct Designs and Vaccine Compositions

In one aspect, provided herein are compositions and methods for augmenting, inducing, promoting, enhancing or improving an immune response against a virus (e.g., as described herein). In one embodiment, the composition and methods described here are designed to augment, induce, promote, enhance or improve immunological memory against a virus (e.g., as described herein). In one embodiment, the composition and methods described here are designed to act as immunological boost to a primary vaccine, such as a vaccine directed to an antigenic polypeptide of a virus. In one embodiments, the composition comprises one or more polynucleotide constructs (designated herein as “Strings”) that encode one or more antigenic epitopes of one or more viruses. Both coding and non-coding strands are contemplated herein. In some embodiments, the strings refer to polynucleotide chains that encode a plurality of antigenic epitopes of one or more viruses in tandem. In some embodiments there are about 2 to about 100, about 2 to about 1000 or about 2 to about 10,000 epitopes encoded in one string. In some embodiments about 2-5000 antigenic epitopes of one or more viruses are encoded in one polynucleotide string. In some embodiments about 2-4000 antigenic epitopes of one or more viruses are encoded in one polynucleotide string. In some embodiments about 2-3000 antigenic epitopes of one or more viruses are encoded in one polynucleotide string. In some embodiments about 2-2000 antigenic epitopes of one or more viruses are encoded in one polynucleotide string. In some embodiments about 2-1000 antigenic epitopes of one or more viruses are encoded in one polynucleotide string. In some embodiments about 10-500 antigenic epitopes of one or more viruses are encoded in one polynucleotide string. In some embodiments about 10-200 antigenic epitopes of one or more viruses are encoded in one polynucleotide string. In some embodiments about 20-100 antigenic epitopes of one or more viruses are encoded in one polynucleotide string. In some embodiments, the virus is or comprises a coronavirus.

In one aspect, provided herein are compositions and methods for augmenting, inducing, promoting, enhancing or improving an immune response against 2019 SARS CoV-2 virus. In one embodiment, the composition and methods described here are designed to augment, induce, promote, enhance or improve immunological memory against 2019 SARS CoV-2 virus. In one embodiment, the composition and methods described here are designed to act as immunological boost to a primary vaccine, such as a vaccine directed to a spike protein of the 2019 SARS CoV-2 virus. In one embodiments, the composition comprises one or more polynucleotide constructs (designated herein as “Strings”) that encode one or more SARS CoV-2 epitopes. Both coding and non-coding strands are contemplated herein. In some embodiments, the strings refer to polynucleotide chains that encode a plurality of SARS CoV-2 epitopes in tandem. In some embodiments there are about 2 to about 100, about 2 to about 1000 or about 2 to about 10,000 epitopes encoded in one string. In some embodiments about 2-5000 SARS CoV-2 epitopes are encoded in one polynucleotide string. In some embodiments about 2-4000 SARS CoV-2 epitopes are encoded in one polynucleotide string. In some embodiments about 2-3000 SARS CoV-2 epitopes are encoded in one polynucleotide string. In some embodiments about 2-2000 SARS CoV-2 epitopes are encoded in one polynucleotide string. In some embodiments about 2-1000 SARS CoV-2 epitopes are encoded in one polynucleotide string. In some embodiments about 10-500 SARS CoV-2 epitopes are encoded in one polynucleotide string. In some embodiments about 10-200 SARS CoV-2 epitopes are encoded in one polynucleotide string. In some embodiments about 20-100 SARS COV-2 epitopes are encoded in one polynucleotide string.

In some embodiments the SARS CoV-2 epitopes encoded by the string constructs comprise epitopes that are predicted by a HLA binding and presentation prediction software to be of high likelihood to be presented by a protein encoded by an HLA to a T cell for eliciting immune response. In some embodiments the SARS COV-2 epitopes encoded by the string constructs that are predicted to have a high likelihood to be presented by a protein encoded by an HLA, are selected from any one of the proteins or peptides described in Tables 1-12, 14A, 14B and 15. In some embodiments the SARS CoV-2 epitopes encoded by the string constructs comprise epitopes that are predicted to have a high likelihood to be presented by a protein encoded by an HLA, and the epitope is selected from any one of the proteins described in Table 1A, Table 1B, Table 1C, Table 2Ai, Table 2Aii, Table 2B, Table 9, Table 10, Table 11, Table 12, Table 14A, Table 14B and/or Table 15. In some embodiments, the epitopes in a string construct comprise nucleocapsid epitopes.

In some embodiments, the epitopes in a string construct comprise spike(S) epitopes. In some embodiments, the epitopes in a string construct comprise nucleocapsid epitopes. In some embodiments, the epitopes in a string construct comprise membrane protein epitopes. In some embodiments, the eptipoes in a string construct comprises ORF1ab epitopes. In some embodiments, the epitopes in a string construct comprise NSP1, NSP2, NSP3, or NSP4 epitopes. In some embodiments, the string constructs comprise a multitude of epitopes that are from 2, 3, 4, or more proteins in a virus. In some embodiments the string constructs comprise the features described in Tables 9-12, and 15. In some embodiments the String constructs comprise a sequence as depicted in SEQ ID RS C1n, RS C2n, RS C3n, RS C4n, SEQ ID RS C5n, RS Con, RS C7n, RS C8n or a sequence that has at least 70% sequence identity to any one of the sequences depicted in SEQ ID RS Cin, RS C2n, RS C3n, RS C4n, SEQ ID RS C5n, RS Con, RS C7n, RS C8n. In some embodiments, the string constructs comprise additional sequences such as linkers, and sequences encoding peptide autocleavage sequences, for example, T2A, or P2A sequences. In some embodiments the string constructs comprises two or more overlapping epitope sequences. In some embodiments a String construct comprise a sequence that is 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the sequences SEQ ID RS Cin, RS C2n, RS C3n, RS C4n, SEQ ID RS C5n, RS Con, RS C7n, RS C8n.

In some embodiments, the epitopes are arranged on a string to maximize immunogenicity of the string, for example by maximizing recognition by HLA allele repertoire of a subject. In some embodiments, the same string encodes epitopes that can bind to or are predicted to bind to different HLA alleles. For instance, as is well exemplified in the sequences tables, e.g., at least in Tables 9, 10, 11, 12, 14A and 14B, and 15, a string may encode epitope(s) that comprise: (a) a first epitope that binds to or is predicted to bind to a first MHC peptide encoded by a first HLA allele; (b) a second epitope that binds to or is predicted to bind to a second MHC peptide encoded by a second HLA allele; (c) a third epitope that binds to or is predicted to bind to a third MHC peptide encoded by a third HLA allele- and more such epitopes can be added, as in for example in sting sequences of RS-C1, or RS-C2 etc.; wherein the first, second and third epitopes are epitopes from the same viral protein, or from different viral proteins. In this way, the epitope distribution encoded by a single string is maximized for hitting the different MHC based presentation to T cells, thereby maximizing the probability of generating an antiviral response from a wider range of patients in the given population and the robustness of the response of each patent. In some embodiments, the epitopes are selected on the basis of high scoring prediction for binding to an HLA by a reliable prediction algorithm or system, such as the RECON prediction algorithm. In some embodiments, the present disclosure provides an insight that particularly successful strings can be provided by selecting epitopes based on highly reliable and efficient prediction algorithm, in the layout of the epitopes encoded by the string, with or without non-epitope sequences or sequences flanking the epitopes, and is such that the immunogenicity of the string is validated in an ex vivo cell culture model, or in an animal model, specifically in showing T cell induction following vaccination with a string construct or a polypeptide encoded by a string construct with the finding of epitope specific T cell response. In some embodiments, the validation may be from using in human patients, and with a finding that T cells obtained from a patient post vaccination shows epitope specific efficient and lasting T cell response. In one embodiment, the efficiency of a string as a vaccine is influenced by its design, that in part depends on strength of the bioinformatic information used in the thoughtful execution of the design, the reliability of the MHC presentation prediction model, the efficiency of epitope processing when a string vaccine is expressed in a cell, among others.

In some embodiments the epitope-coding sequences in a string construct are flanked by one or more sequences selected for higher immunogenicity, better cleavability for peptide presentation to MHCs, better expression, and/or improved translation in a cell in a subject. The flanking sequences may comprise a linker with a specific cleavable sequences. In some embodiments the epitope-coding sequences in a string construct are flanked by a secretory protein sequence. In some embodiments a string sequence encodes an epitope that may comprise or otherwise be linked to a secretory sequence such as MFVFLVLLPLVSSQCVNLT, or at least a sequence having 1, 2, 3, 4, or at the most 5 amino acid differences relative thereto. In some embodiments, a string sequence encodes an epitope that may be linked at the N-terminal end by a sequence MFVFLVLLPLVSSQCVNLT or a sequence having 1, 2, 3, 4, or at the most 5 amino acid differences relative thereto. The linked sequences may comprise a linker with a specific cleavable sequences. In some embodiments the string construct is linked to a transmembrane domain (TM). In some embodiments, a string sequence encodes an epitope that may be linked at the C terminal sequence by a TM domain sequence EQYIKWPWYIWLGFIAGLIAIVMVTIMLCCMTSCCSCLKGCCSCGSCCKFDEDDSEPVLKGVKL HYT, or a sequence having 1, 2, 3, 4, or at the most 5 amino acid differences relative thereto. In some embodiments, one or more linker sequences may comprise cleavage sequences. In some embodiments, a linker may have a length of 2, 3, 4, 5, 6, 7, 8, 9, 10 or more amino acid. In some embodiments a linker of not more than about 30, 25, 20, 15, 10 or fewer amino acids is used. In general, any amino acid may be present as a linker sequence. In some embodiments, a linker or cleavage sequence contains a lysine (K). In some embodiments, a linker or cleavage sequence contains an arginine (R). In some embodiments, a linker or cleavage sequence contains a methionine (M). In some embodiments, a linker or cleavage sequence contains a tyrosine (Y). In some embodiments, a linker is designed to comprise amino acids based on a cleavage predictor to generate highly-cleavable sequences peptide sequences, and is a novel and effective way of delivering immunogenic T cell epitopes in a T cell vaccine setting. In some embodiments, the epitope distribution and their juxtaposition encoded in a string construct are so designed to facilitate cleavage sequences contributed by the amino acid sequences of the epitopes and/or the flanking or linking residues and thereby using minimal linker sequences. In some embodiments, a linker sequence comprises a 4 amino acid-linker sequence that induces proteosomal cleavage. Some exemplary cleavage sequences may be one or more of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA. Among other things, MS data included herein demonstrates that the epitopes that are highly predicted for binding ended up being presented to T cells, and immunogenic. In some embodiments, a linker sequence included in a string construct is enriched in glycine (G) and/or serine(S). For example, in some embodiments, such a linker sequence comprises at least 50% (including, e.g., at least 60%, at least 70%, at least 80%, at least 90%, at least 95%, or more and up to 100%) G and/or S residues. In some embodiments, such a linker sequence comprises GGSGGGGSGG or GGSLGGGGSG. In some embodiments, a linker sequence enriched in G and/or S may be included between an antigenic region and a non-antigenic region (e.g., between a secretory signal and an antigenic region and/or between a transmembrane domain and an antigenic region).

In some embodiments the string constructs may be mRNA. In some embodiments a pharmaceutical composition may comprise one or more mRNA string construct, each comprising a sequence encoding a plurality of coronavirus (e.g., in some embodiments SARS-CoV-2) epitopes. In some embodiments, the one or more mRNA may comprise a plurality of epitopes from a coronavirus (e.g., in some embodiments SARS-CoV-2) spike protein, wherein each of the plurality of epitopes is predicted by an HLA binding and presentation prediction algorithm to be of high likelihood to be presented by a protein encoded by an HLA to a T cell for eliciting immune response. In some embodiments, the one or more mRNA may comprise a plurality of epitopes from a coronavirus (e.g., in some embodiments SARS-CoV-2) membrane protein, wherein each of the plurality of epitopes is predicted by an HLA binding and presentation prediction algorithm to be of high likelihood to be presented by a protein encoded by an HLA to a T cell for eliciting immune response. In some embodiments, the one or more mRNA may comprise a plurality of epitopes from a coronavirus (e.g., in some embodiments SARS-CoV-2) ORF1ab, wherein each of the plurality of epitopes is predicted by an HLA binding and presentation prediction algorithm to be of high likelihood to be presented by a protein encoded by an HLA to a T cell for eliciting immune response. In some embodiments the one or more mRNA may comprise a plurality of epitopes from a coronavirus (e.g., SARS-CoV-2) nucleocapsid protein, wherein each of the plurality of epitopes is predicted by an HLA binding and presentation prediction algorithm to be of high likelihood to be presented by a protein encoded by an HLA to a T cell for eliciting immune response. In some embodiments the one or more mRNA may comprise a plurality of epitopes from the coronavirus (e.g., SARS-CoV-2) spike, or ORF1ab (e.g., one or more non-structural proteins), or nucleocapsid protein, or membrane protein or any other protein wherein each of the plurality of epitopes is predicted by an HLA binding and presentation prediction algorithm to be of high likelihood to be presented by a protein encoded by an HLA to a T cell for eliciting immune response. In some embodiments the one or more mRNA may comprise a plurality of epitopes from at least two of the following: the coronavirus (e.g., SARS-CoV-2) spike, or ORF1ab (e.g., one or more non-structural proteins), or nucleocapsid protein, or membrane protein or any other protein wherein each of the plurality of epitopes is predicted by an HLA binding and presentation prediction algorithm to be of high likelihood to be presented by a protein encoded by an HLA to a T cell for eliciting immune response. In some embodiments the one or more mRNA may comprise a plurality of epitopes from coronavirus (e.g., SARS-CoV-2) ORF1ab (e.g., one or more non-structural proteins), nucleocapsid protein, and membrane protein, wherein each of the plurality of epitopes is predicted by an HLA binding and presentation prediction algorithm to be of high likelihood to be presented by a protein encoded by an HLA to a T cell for eliciting immune response. In some embodiments the plurality of epitopes may comprise epitopes from a single 2019 SARS CoV-2 protein. In some embodiments the plurality of epitopes may comprise epitopes from multiple 2019 SARS CoV-2 protein. In some embodiments the plurality of epitopes may comprise epitopes from 2019 SARS CoV-2 nucleocapsid protein.

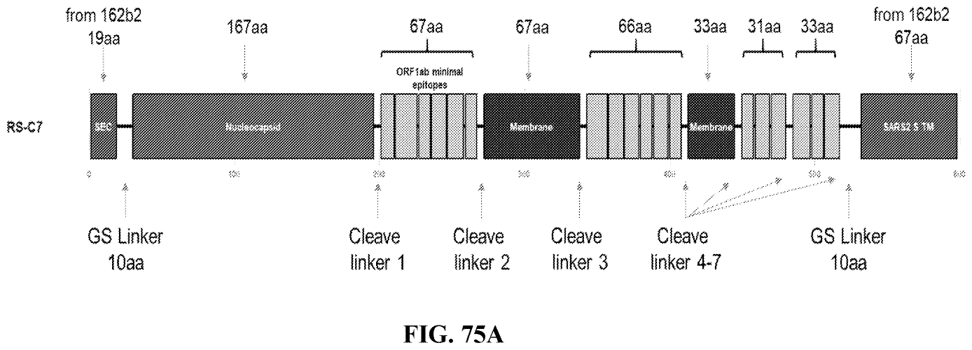

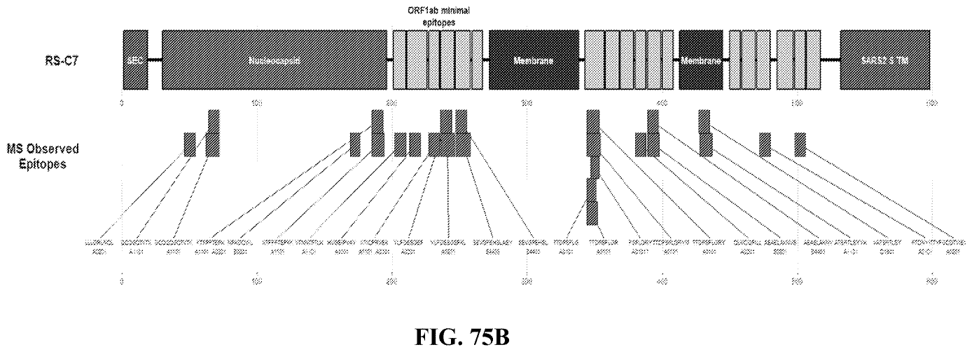

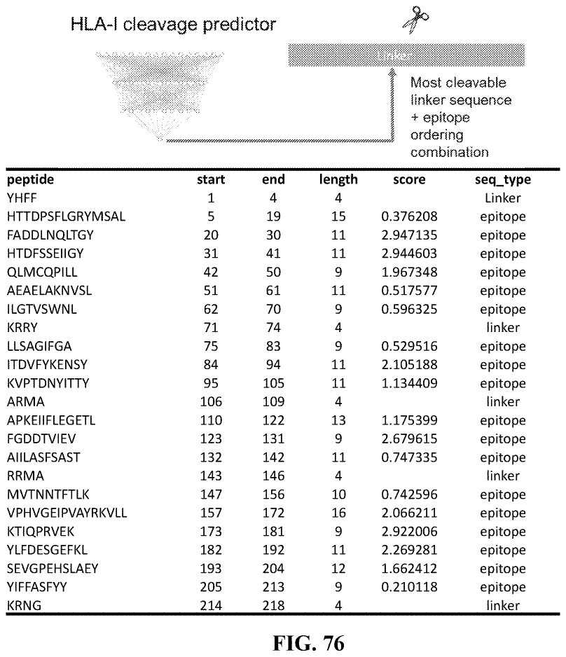

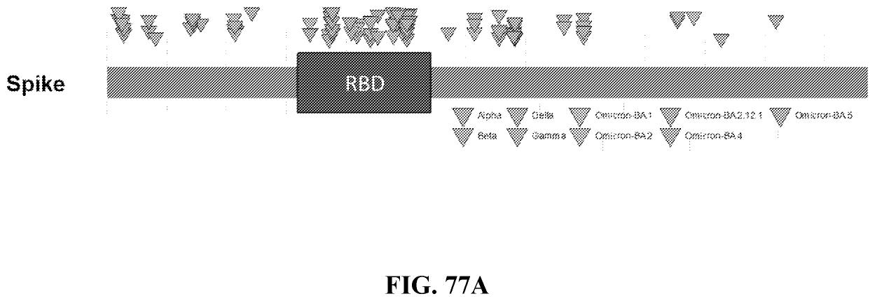

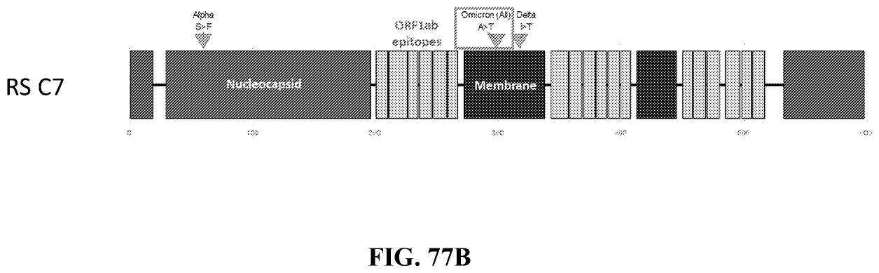

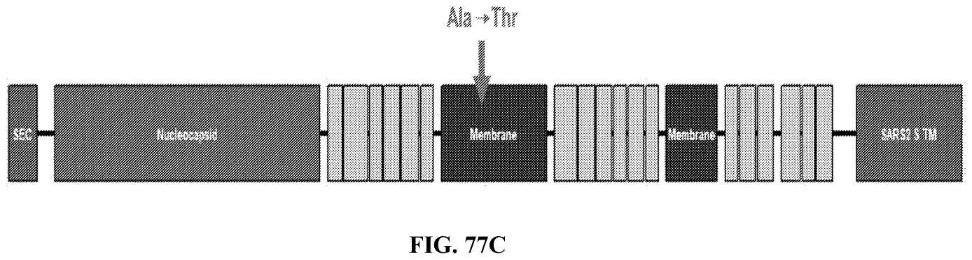

In some embodiments, a provided mRNA string construct comprises a nucleotide sequence that encodes a polyepitopic polypeptide comprising at least two or more antigenic segments (e.g., from nucleocapsid and/or membrane proteins) interspersed with at least one (including, e.g., at least two, at least three, at least four, at least five, at least six, or more) short epitope of ORF1ab protein. In some embodiments, such a provided mRNA string construct further comprises a nucleotides that encodes a cleavable linker sequence (e.g., in some embodiments a nucleotide sequence encoding a 4 amino acid-linker sequence that induces proteosomal cleavage as described herein, e.g., a cleavable linker sequence independently selected from FKAA, KRNG, YNSF, KKNG, YRSY, RRRG, and ARMA) that separates each antigenic segment and the short epitopes of ORF1ab protein. See for example, FIG. 75A. In some embodiments, such a provided mRNA string construct comprises a nucleotide sequence that encodes a polyepitopic polypeptide comprising two or more antigenic segments (e.g., from nucleocapsid and/or membrane proteins) interspersed with at least two short epitopes of ORF1ab protein, wherein the at least two short epitopes of ORF1ab are separated by a cleavable linker sequence (e.g., a 4 amino acid linker sequence that induces proteasomal cleavage, e.g., a sequenece selected from FKAA, KRNG, YNSF, KKNG, YRSY, RRRG, and ARMA). In some embodiments, such a cleavable linker sequence between the short epitopes of ORF1ab protein is or comprise a sequence of ARMA.

In some embodiments, the encoded antigenic segments (e.g., from nucleocapsid and/or membrane proteins) can each independently have a length of about 10 amino acids to about 500 amino acids, about 25 amino acids to about 250 amino acids, or about 25 amino acids to 200 amino acids. In some embodiments, a plurality of (e.g., at least two or more including, e.g., at least three, at least four, at least five, at least six, or more) encoded short eptiopes of ORF1ab protein form a polyepitopic segment having a length of about 10 amino acids to about 500 amino acids, or about 15 amino acids to about 250 amino acids, or about 20 amino acids to about 150 amino acids, or about 20 amino acids to about 100 amino acids. In some embodiments, a polyepitopic polypeptide encoded by a provided mRNA string construct has a length of about 150 amino acids to about 2000 amino acids, or about 300 amino acids to about 1500 amino acids, or about 400 amino acids to about 1000 amino acids.

In some embodiments, the mRNA may comprise a 5′UTR and/or a 3′UTR. In some embodiments, the UTR may comprise a poly A sequence. A poly A sequence may be between 50-200 nucleotides long. In some embodiments the 2019 SARS CoV-2 viral epitopes may be flanked by a signal peptide sequence, e.g., SP1 sequence to enhance epitope processing and presentation. In some embodiments the 2019 SARS CoV-2 viral epitopes are flanked with an MITD sequence to enhance epitope processing and presentation. In some embodiments, the polynucleotide comprises a dEarI-hAg sequence. In some embodiments, the poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments a poly A tail of a string construct may comprise about 200 A residues. In some embodiments a poly A tail of a string construct may comprise 180 A residues or less. In some embodiments a poly A tail of a string construct may comprise about 180 A residues. In some embodiments, the poly A tail may comprise 150 residues or less. In some embodiments a poly A tail of a string construct may comprise about 150 A residues. In some embodiments, the poly A tail may comprise 120 residues or less. In some embodiments a poly A tail of a string construct may comprise about 120 A residues. In some embodiments, the vaccine described herein comprises as the active principle single-stranded RNA that may be translated into the respective protein upon entering cells of a recipient. In addition to wildtype or codon-optimized sequences encoding the antigen sequence, the RNA may contain one or more structural elements optimized for maximal efficacy of the RNA with respect to stability and translational efficiency (5′ cap, 5′ UTR, 3′ UTR, poly(A)-tail). In one embodiment, the RNA contains all of these elements. In one embodiment, beta-S-ARCA (D1) (m₂^{7,2′-O}GppSpG) or a cap comprising a Cap1 structure (e.g., in some embodiments a trinucleotide cap, e.g., m₂^{7,3′-O}Gppp(m₁^{2′-O})ApG) may be utilized as specific capping structure at the 5′-end of the RNA drug substances. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA. Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency. In some embodiments, a string construct described herein comprises one or more of the following features as described in WO2021/213924: a 5′ cap, a 3′ UTR, a 5′UTR, and a poly(A) tail. In some embodiments, a string construct described herein comprises each of the following features as utilized in BNT162b2 (e.g., as described in WO2021/213924, the entire content of which is incorporated herein by reference for purposes described herein).

In some embodiments the nucleotide sequence of the string constructs, encoding the plurality of epitopes, may be codon optimized. An example of a codon optimized sequence may be a sequence optimized for expression in a eukaryote, e.g., humans (i.e. being optimized for expression in humans), or for another eukaryote, animal or mammal. Codon optimization for a host species other than human, or for codon optimization for specific organs is known. In some embodiments, the coding sequence encoding a protein may be codon optimized for expression in eukaryotic cells, such as human cells. Codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are readily available, for example, at the “Codon Usage Database” available at kazusa.orjp/codon/and these tables may be adapted in a number of ways. Computer algorithms for codon optimizing a particular sequence for expression in a particular host cell are also available, such as Gene Forge (Aptagen; Jacobus, PA), are also available.

In some embodiments, the stability and translation efficiency of RNA may incorporate one or more elements established to contribute to stability and/or translation efficiency of RNA; exemplary such elements are described, for example, in PCT/EP2006/009448 incorporated herein by reference. In order to increase expression of the RNA used according to the present disclosure, it may be modified within the coding region, i.e. the sequence encoding the expressed peptide or protein, without altering the sequence of the expressed peptide or protein, so as to increase the GC-content to increase mRNA stability and to perform a codon optimization and, thus, enhance translation in cells.

In some embodiments, the string construct may comprise an F element. In some embodiments, the F element sequence is a 3 UTR of amino-terminal enhancer of split (AES).

In some embodiments a String mRNA construct as described above may comprise 1, 2, 3, 4, 5, 6, 7, 8, 9, 10, 11, 12, 13, 14, 15 or more epitopes. In some embodiments the pharmaceutical composition comprises 2, 3, 4, 5, 6, 7, 8, 9, or 10 or more strings. In some embodiments the pharmaceutical composition comprises 6 strings. In some embodiments the pharmaceutical composition comprises 7 strings. In some embodiments the pharmaceutical composition comprises 8 strings. In some embodiments the pharmaceutical composition comprises 9 strings. In some embodiments the pharmaceutical composition comprises 10 strings.

In some embodiments a string construct may be a polynucleotide, wherein the polynucleotide is DNA.

In some embodiments the pharmaceutical composition comprising one or more String mRNA construct as described above may be encapsulated in a lipid nanoparticle. As used in the present disclosure, “nanoparticle” refers to a particle having an average diameter suitable for parenteral administration. A lipid nanoparticle (LNP) may be 100-250 nm in diameter. In some embodiments, a plurality of lipid nanoparticles may have an average particle size of less than 200 nm, less than 150 nm, less than 100 nm, less than 80 nm, less than 75 nm, or lower. In some embodiments, a plurality of lipid nanoparticles may have an average particle size of at least 30 nm, at least 50 nm, at least 60 nm, at least 70 nm, at least 80 nm, at least 90 nm, at least 100 nm, at least 125 nm, at least 150 nm, or more. Combinations of the above-mentioned ranges are also possible. In some embodiments, a plurality of lipid nanoparticles may have an average particle size of 30 nm to 200 nm, or 30 nm to 100 nm or 50 nm to 80 nm, or 50 nm to less than 80 nm. In some embodiments, an LNP may comprise a cationic lipid. An LNP may comprise a non-cationic lipid. An LNP may comprise a PEG-modified lipid. An LNP may comprise a sterol or a steroidal lipid.

In one embodiment, the delivery particles are lipoplex (LPX) particles. In one embodiment, the RNA lipoplex particles are obtainable by mixing the RNA with liposomes. In one embodiment, the RNA lipoplex particles are obtainable by mixing the RNA with lipids.

In one embodiment, the LNP particles comprise ((4-hydroxybutyl) azanediyl)bis(hexane-6,1-diyl)bis(2-hexyldecanoate), 2-[(polyethylene glycol)-2000]-N,N-ditetradecylacetamide, 1,2-Distearoyl-sn-glycero-3-phosphocholine, and cholesterol. In one embodiment, the RNA is formulated or is to be formulated as colloid. In one embodiment, the RNA is formulated or is to be formulated as particles, forming the dispersed phase of a colloid. In one embodiment, 50% or more, 75% or more, or 85% or more of the RNA are present in the dispersed phase. In one embodiment, the RNA is formulated or is to be formulated as particles comprising RNA and lipids. In one embodiment, the particles are formed by exposing RNA, dissolved in an aqueous phase, with lipids, dissolved in an organic phase. In one embodiment, the organic phase comprises ethanol. In one embodiment, the particles are formed by exposing RNA, dissolved in an aqueous phase, with lipids, dispersed in an aqueous phase. In one embodiment, the lipids dispersed in an aqueous phase form liposomes. In some embodiments the pharmaceutical composition comprising one or more String mRNA construct as described above may be administered with another 2019 SARS COV-2 vaccine, which can be in some embodiments, e.g., protein-based, RNA-based, DNA-based, viral vector-based vaccines, and may be administered either before, after, or simultaneously with. In some embodiments, a pharmaceutical composition comprising one or more String mRNA construct as described above may be administered to a subject in need thereof such that the subject receives a combination of the pharmaceutical composition described herein and an another 2019 SARS CoV-2 vaccine (e.g., a vaccine that induces production of antibodies to SARS CoV-2 protein such as S protein or an immunogenic fragment thereof). For example, in some embodiments, a pharmaceutical composition comprising one or more String mRNA construct as described above may be administered to a subject who is receiving or has received another 2019 SARS CoV-2 vaccine (e.g., a vaccine that induces production of antibodies to 2019 SARS CoV-2 protein such as S protein or an immunogenic fragment thereof).

In some embodiments the pharmaceutical composition comprising one or more String mRNA construct as described above may be co-administered with a vaccine directed against SARS CoV-2 spike protein. In some embodiments, the vaccine comprises a SARS-CoV-2 spike protein of 2019 SARS COV-2 or a nucleic acid sequence encoding the same, for example which may have any of the following specifications:

### Exemplary Construct Encoding a SARS-CoV-2 Spike Protein

Structure m₂^{7,3′-O}Gppp(m₁^{2′-O})ApG)-hAg-Kozak-S1S2-PP-FI-A30L70
Encoded antigen Viral spike protein (S1S2 protein) of the SARS CoV-2 (S1S2 full-length protein, sequence variant)

### Exemplary Nucleotide Sequence Encoding a SARS-CoV-2 Spike Protein

Nucleotide sequence is shown with individual sequence elements as indicated in bold letters. In addition, the sequence of the translated protein is shown in italic letters below the coding nucleotide sequence (*=stop codon). One of skill in the art reading the present disclosure will recognize that, in some embodiments, different mutations known in the art, e.g., proline subsitutions (e.g., as disclosed in WO 2021243122 A2; Hsieh, Ching-Lin, et al. (“Structure-based design of prefusion-stabilized SARS-CoV-2 spikes,” Science 369.6510 (2020): 1501-1505; WO2022/067010 A1; WO2018/081318A1; WO2021/163365A1; and WO2021/213924A1, the contents of each which are incorporated by reference herein in their entirety) and/or furin cleavage site mutations (e.g., as described in WO2021163365 or WO2021243122) can be introduced to S protein to increase expression and/or stabilize a prototypical prefusion conformation.

```
10 20 30 40 50 53
AGAAUAAACU AGUAUUCUUC UGGUCCCCAC AGACUCAGAG AGAACCCGCC ACC
                                                    hAg-Kozak
                63 73 83 93 103 113
AUGUUUGUGU UUCUUGUGCU GCUGCCUCUU GUGUCUUCUC AGUGUGUGAA UUUGACAACA
    M F V F L V L L P L V S S Q C V N L T T
                                                    S protein
              123 133 143 153 163 173
AGAACACAGC UGCCACCAGC UUAUACAAAU UCUUUUACCA GAGGAGUGUA UUAUCCUGAU
    R T Q L P P A Y T N S F T R G V Y Y P D
                                                    S protein
              183 193 203 213 223 233
AAAGUGUUUA GAUCUUCUGU GCUGCACAGC ACACAGGACC UGUUUCUGCC AUUUUUUAGC
    K V F R S S V L H S T Q D I F L P F F S
                                                    S protein
              243 253 263 273 283 293
AAUGUGACAU GGUUUCAUGC AAUUCAUGUG UCUGGAACAA AUGGAACAAA AAGAUUUGAU
    N V T W F H A I H V S G T N G T K R F D
                                                    S protein
              303 313 323 333 343 353
AAUCCUGUGC UGCCUUUUAA UGAUGGAGUG UAUUUUGCUU CAACAGAAAA GUCAAAUAUU
    N P V L P F N D G V Y F A S T E K S N I
                                                    S protein
              363 373 383 393 403 413
AUUAGAGGAU GGAUUUUUGG AACAACACUG GAUUCUAAAA CACAGUCUCU GCUGAUUGUG
    I R G W I F G T T L D S K T Q S L L I V
                                                    S protein
              423 433 443 453 463 473
AAUAAUGCAA CAAAUGUGGU GAUUAAAGUG UGUGAAUUUC AGUUUUGUAA UGAUCCUUUU
    N N A T N V V I K V C E F Q F C N D P F
                                                    S protein
              483 493 503 513 523 533
CUGGGAGUGU AUUAUCACAA AAAUAAUAAA UCUUGGAUGG AAUCUGAAUU UAGAGUGUAU
    L G V Y Y H K N N K S W M E S E F R V Y
                                                    S protein
              543 553 563 573 583 593
UCCUCUGCAA AUAAUUGUAC AUUUGAAUAU GUGUCUCAGC CUUUUCUGAU GGAUCUGGAA
    S S A N N C T F E Y V S Q P F L M D L E
                                                    S protein
              603 613 622 633 643 653
GGAAAACAGG GCAAUUUUAA AAAUCUGAGA GAAUUUGUGU UUAAAAAUAU UGAUGGAUAU
    G K Q G N F K N L R E F V F K N I D G Y
                                                    S protein
              663 673 683 693 703 713
UUUAAAAUUU AUUCUAAACA CACACCAAUU AAUUUAGUGA GAGAUCUGCC UCAGGGAUUU
    F K I Y S K H T P I N L V R D L P Q G F
                                                    S protein
              723 733 743 753 763 773
UCUGCUCUGG AACCUCUGGU GGAUCUGCCA AUUGGCAUUA AUAUUACAAG AUUUCAGACA
    S A L E P L V D L P I G I N I T R F Q T
                                                    S protein
              783 793 803 813 823 833
CUGCUGGCUC UGCACAGAUC UUAUCUGACA CCUGGAGAUU CUUCUUCUGG AUGGACAGCC
    L L A L H R S Y L T P G D S S S G W T A
                                                    S protein
              843 853 863 873 883 893
GGAGCUGCAG CUUAUUAUGU GGGCUAUCUG CAGCCAAGAA CAUUUCUGCU GAAAUAUAAU
    G A A A Y Y V G Y L Q P R T F L L K Y N
                                                    S protein
              903 913 923 933 943 953
GAAAAUGGAA CAAUUACAGA UGCUGUGGAU UGUGCUCUGG AUCCUCUGUC UGAAACAAAA
    E N G T I T D A V D C A L D P L S E T K
                                                    S protein
              963 973 983 993 1003 1013
UGUACAUUAA AAUCUUUUAC AGUGGAAAAA GGCAUUUAUC AGACAUCUAA UUUUAGAGUG
    C T L K S F T V E K G I Y Q T S N F R V
                                                    S protein
            1023 1033 1043 1053 1063 1073
CAGCCAACAG AAUCUAUUGU GAGAUUUCCA AAUAUUACAA AUCUGUGUCC AUUUGGAGAA
    Q P T E S I V R F P N I T N L C P F G E
                                                    S protein
            1083 1093 1103 1113 1123 1133
GUGUUUAAUG CAACAAGAUU UGCAUCUGUG UAUGCAUGGA AUAGAAAAAG AAUUUCUAAU
    V F N A T R F A S V Y A W N R K R I S N
                                                    S protein
            1143 1153 1163 1173 1183 1193
UGUGUGGCUG AUUAUUCUGU GCUGUAUAAU AGUGCUUCUU UUUCCACAUU UAAAUGUUAU
    C V A D Y S V L Y N S A S F S T F K C Y
                                                    S protein
            1203 1213 1223 1233 1243 1253
GGAGUGUCUC CAACAAAAUU AAAUGAUUUA UGUUUUACAA AUGUGUAUGC UGAUUCUUUU
    G V S P T K L N D L C F T N V Y A D S F
                                                    S protein
            1263 1273 1283 1293 1303 1313
GUGAUCAGAG GUGAUGAAGU GAGACAGAUU GCCCCCGGAC AGACAGGAAA AAUUGCUGAU
    V I R G D E V R Q I A P G Q T G K I A D
                                                    S protein
            1323 1333 1343 1353 1363 1373
UACAAUUACA AACUGCCUGA UGAUUUUACA GGAUGUGUGA UUGCUUGGAA UUCUAAUAAU
    Y N Y K L P D D F T G C V I A W N S N N
                                                    S protein
            1383 1393 1403 1413 1423 1433
UUAGAUUCUA AAGUGGGAGG AAAUUACAAU UAUCUGUACA GACUGUUUAG AAAAUCAAAU
    L D S K V G G N Y N Y L Y R L F R K S N
                                                    S protein
            1443 1453 1463 1473 1483 1493
CUGAAACCUU UUGAAAGAGA UAUUUCAACA GAAAUUUAUC AGGCUGGAUC AACACCUUGU
    L K P F E R D I S T E I Y Q A G S T P C
                                                    S protein
            1503 1513 1523 1533 1543 1553
AAUGGAGUGG AAGGAUUUAA UUGUUAUUUU CCAUUACAGA GCUAUGGAUU UCAGCCAACC
    N G V E G F N C Y F P L Q S Y G F Q P T
                                                    S protein
            1563 1573 1583 1593 1603 1613
AAUGGUGUGG GAUAUCAGCC AUAUAGAGUG GUGGUGCUGU CUUUUGAACU GCUGCAUGCA
    N G V G Y Q P Y R V V V L S F E L L H A
                                                    S protein
            1623 1633 1643 1653 1663 1673
CCUGCAACAG UGUGUGGACC UAAAAAAUCU ACAAAUUUAG UGAAAAAUAA AUGUGUGAAU
    P A T V C G P K K S T N L V K N K C V N
                                                    S protein
            1683 1693 1703 1713 1723 1733
UUUAAUUUUA AUGGAUUAAC AGGAACAGGA GUGCUGACAG AAUCUAAUAA AAAAUUUCUG
    F N F N G L T G T G V L T E S N K K F L
                                                    S protein
            1743 1753 1763 1773 1783 1793
CCUUUUCAGC AGUUUGGCAG AGAUAUUGCA GAUACCACAG AUGCAGUGAG AGAUCCUCAG
    P F Q Q F G R D I A D T T D A V R D P Q
                                                    S protein
            1803 1813 1823 1833 1843 1853
ACAUUAGAAA UUCUGGAUAU UACACCUUGU UCUUUUGGGG GUGUGUCUGU GAUUACACCU
    T L E I L D I T P C S F G G V S V I T P
                                                    S protein
            1863 1873 1883 1893 1903 1913
GGAACAAAUA CAUCUAAUCA GGUGGCUGUG CUGUAUCAGG AUGUGAAUUG UACAGAAGUG
    G T N T S N Q V A V L Y Q D V N C T E V
                                                    S protein
            1923 1933 1943 1953 1963 1973
CCAGUGGCAA UUCAUGCAGA UCAGCUGACA CCAACAUGGA GAGUGUAUUC UACAGGAUCU
    P V A I H A D Q L T P T W R V Y S T G S
                                                    S protein
            1983 1993 2003 2013 2023 2033
AAUGUGUUUC AGACAAGAGC AGGAUGUCUG AUUGGAGCAG AACAUGUGAA UAAUUCUUAU
    N V F Q T R A G C L I G A E H V N N S Y
                                                    S protein
            2043 2053 2063 2073 2083 2093
GAAUGUGAUA UUCCAAUUGG AGCAGGCAUU UGUGCAUCUU AUCAGACACA GACAAAUUCC
    E C D I P I G A G I C A S Y Q T Q T N S
                                                    S protein
            2103 2113 2123 2133 2143 2153
CCAAGGAGAG CAAGAUCUGU GGCAUCUCAG UCUAUUAUUG CAUACACCAU GUCUCUGGGA
    P R R A R S V A S Q S I I A Y T M S L G
                                                    S protein
            2163 2173 2183 2193 2203 2213
GCAGAAAAUU CUGUGGCAUA UUCUAAUAAU UCUAUUGCUA UUCCAACAAA UUUUACCAUU
    A E N S V A Y S N N S I A I P T N F T I
                                                    S protein
            2223 2233 2243 2253 2263 2273
UCUGUGACAA CAGAAAUUUU ACCUGUGUCU AUGACAAAAA CAUCUGUGGA UUGUACCAUG
    S V T T E I L P V S M T K T S V D C T M
                                                    S protein
            2283 2293 2303 2313 2323 2333
UACAUUUGUG GAGAUUCUAC AGAAUGUUCU AAUCUGCUGC UGCAGUAUGG AUCUUUUUGU
    Y I C G D S T E C S N L L L Q Y G S F C
                                                    S protein
            2343 2353 2363 2373 2383 2393
ACACAGCUGA AUAGAGCUUU AACAGGAAUU GCUGUGGAAC AGGAUAAAAA UACACAGGAA
    T Q L N R A L T G I A V E Q D K N T Q E
                                                    S protein
            2403 2413 2423 2433 2443 2453
GUGUUUGCUC AGGUGAAACA GAUUUACAAA ACACCACCAA UUAAAGAUUU UGGAGGAUUU
    V F A Q V K Q I Y K T P P I K D F G G F
                                                    S protein
            2463 2473 2483 2493 2503 2513
AAUUUUAGCC AGAUUCUGCC UGAUCCUUCU AAACCUUCUA AAAGAUCUUU UAUUGAAGAU
    N F S Q I L P D P S K P S K R S F I E D
                                                    S protein
            2523 2533 2543 2553 2563 2573
CUGCUGUUUA AUAAAGUGAC ACUGGCAGAU GCAGGAUUUA UUAAACAGUA UGGAGAUUGC
    L L F N K V T L A D A G F I K Q Y G D C
                                                    S protein
            2583 2593 2603 2613 2623 2633
CUGGGUGAUA UUGCUGCAAG AGAUCUGAUU UGUGCUCAGA AAUUUAAUGG ACUGACAGUG
    L G D I A A R D L I C A Q K F N G L T V
                                                    S protein
            2643 2653 2663 2673 2683 2693
CUGCCUCCUC UGCUGACAGA UGAAAUGAUU GCUCAGUACA CAUCUGCUUU ACUGGCUGGA
    L P P L L T D E M I A Q Y T S A L L A G
                                                    S protein
            2703 2713 2723 2733 2743 2753
ACAAUUACAA GCGGAUGGAC AUUUGGAGCU GGAGCUGCUC UGCAGAUUCC UUUUGCAAUG
    T I T S G W T F G A G A A L Q I P F A M
                                                    S protein
            2763 2773 2783 2793 2803 2813
CAGAUGGCUU ACAGAUUUAA UGGAAUUGGA GUGACACAGA AUGUGUUAUA UGAAAAUCAG
    Q M A Y R F N G I G V T Q N V L Y E N Q
                                                    S protein
            2823 2833 2843 2853 2863 2873
AAACUGAUUG CAAAUCAGUU UAAUUCUGCA AUUGGCAAAA UUCAGGAUUC UCUGUCUUCU
    K L I A N Q F N S A I G K I Q D S L S S
                                                    S protein
            2883 2893 2903 2913 2923 2933
ACAGCUUCUG CUCUGGGAAA ACUGCAGGAU GUGGUGAAUC AGAAUGCACA GGCACUGAAU
    T A S A L G K L Q D V V N Q N A Q A L N
                                                    S protein
            2943 2953 2963 2973 2983 2993
ACUCUGGUGA AACAGCUGUC UAGCAAUUUU GGGGCAAUUU CUUCUGUGCU GAAUGAUAUU
    T L V K Q L S S N F G A I S S V L N D I
                                                    S protein
            3003 3013 3023 3033 3043 3053
CUGUCUAGAC UGGAUCCUCC UGAAGCUGAA GUGCAGAUUG AUAGACUGAU CACAGGAAGA
    L S R L D P P E A E V Q I D R L I T G R
                                                    S protein
            3063 3073 3083 3093 3103 3113
CUGCAGUCUC UGCAGACUUA UGUGACACAG CAGCUGAUUA GAGCUGCUGA AAUUAGAGCU
    L Q S L Q T Y V T Q Q L I R A A E I R A
                                                    S protein
            3123 3133 3143 3153 3163 3173
UCUGCUAAUC UGGCUGCUAC AAAAAUGUCU GAAUGUGUGC UGGGACAGUC AAAAAGAGUG
    S A N L A A T K M S E C V L G Q S K R V
                                                    S protein
            3183 3193 3203 3213 3223 3233
GAUUUUUGUG GAAAAGGAUA UCAUCUGAUG UCUUUUCCAC AGUCUGCUCC ACAUGGAGUG
    D F C G K G Y H L M S F P Q S A P H G V
                                                    S protein
            3243 3253 3263 3273 3283 3293
GUGUUUUUAC AUGUGACAUA UGUGCCAGCA CAGGAAAAGA AUUUUACCAC AGCACCAGCA
    V F L H V T Y V P A Q E K N F T T A P A
                                                    S protein
            3303 3313 3323 3333 3343 3353
AUUUGUCAUG AUGGAAAAGC ACAUUUUCCA AGAGAAGGAG UGUUUGUGUC UAAUGGAACA
    I C H D G K A H F P R E G V F V S N G T
                                                    S protein
            3363 3373 3383 3393 3403 3413
CAUUGGUUUG UGACACAGAG AAAUUUUUAU GAACCUCAGA UUAUUACAAC AGAUAAUACA
    H W F V T Q R N F Y E P Q I I T T D N T
                                                    S protein
            3423 3433 3443 3453 3463 3473
UUUGUGUCAG GAAAUUGUGA UGUGGUGAUU GGAAUUGUGA AUAAUACAGU GUAUGAUCCA
    F V S G N C D V V I G I V N N T V Y D P
                                                    S protein
            3483 3493 3503 3513 3523 3533
CUGCAGCCAG AACUGGAUUC UUUUAAAGAA GAACUGGAUA AAUAUUUUAA AAAUCACACA
    L Q P E L D S F K E E L D K Y F K N H T
                                                    S protein
            3543 3553 3563 3573 3583 3593
UCUCCUGAUG UGGAUUUAGG AGAUAUUUCU GGAAUCAAUG CAUCUGUGGU GAAUAUUCAG
    S P D V D L G D I S G I N A S V V N I Q
                                                    S protein
            3603 3613 3623 3633 3643 3653
AAAGAAAUUG AUAGACUGAA UGAAGUGGCC AAAAAUCUGA AUGAAUCUCU GAUUGAUCUG
    K E I D R L N E V A K N L N E S L I D L
                                                    S protein
            3663 3673 3683 3693 3703 3713
CAGGAACUUG GAAAAUAUGA ACAGUACAUU AAAUGGCCUU GGUACAUUUG GCUUGGAUUU
    Q E L G K Y E Q Y I K W P W Y I W L G F
                                                    S protein
            3723 3733 3743 3753 3763 3773
AUUGCAGGAU UAAUUGCAAU UGUGAUGGUG ACAAUUAUGU UAUGUUGUAU GACAUCAUGU
    I A G L I A I V M V T I M L C C M T S C
                                                    S protein
            3783 3793 3803 3813 3823 3833
UGUUCUUGUU UAAAAGGAUG UUGUUCUUGU GGAAGCUGUU GUAAAUUUGA UGAAGAUGAU
    C S C L K G C C S C G S C C K F D E D D
                                                    S protein
            3843 3853 3863 3873 3878
UCUGAACCUG UGUUAAAAGG AGUGAAAUUG CAUUACACAU GAUGA
    S E P V L K G V K L H Y T * *
                                                    S protein
            3888 3898 3908 3918 3928 3938
CUCGAGCUGG UACUGCAUGC ACGCAAUGCU AGCUGCCCCU UUCCCGUCCU GGGUACCCCG
                                                    FI element
            3948 3958 3968 3978 3988 3998
AGUCUCCCCC GACCUCGGGU CCCAGGUAUG CUCCCACCUC CACCUGCCCC ACUCACCACC
                                                    FI element
            4008 4018 4028 4038 4048 4058
UCUGCUAGUU CCAGACACCU CCCAAGCACG CAGCAAUGCA GCUCAAAACG CUUAGCCUAG
                                                    FI element
            4068 4078 4088 4098 4108 4118
CCACACCCCC ACGGGAAACA GCAGUGAUUA ACCUUUAGCA AUAAACGAAA GUUUAACUAA
                                                    FI element
            4128 4138 4148 4158 4168 4173
GCUAUACUAA CCCCAGGGUU GGUCAAUUUC GUGCCAGCCA CACCCUGGAG CUAGC
                                                    FI element
            4183 4193 4203 4213 4223 4233
AAAAAAAAAA AAAAAAAAAA AAAAAAAAAA GCAUAUGACU AAAAAAAAAA AAAAAAAAAA
                                                    Poly(A)
            4243 4253 4263 4273 4283
AAAAAAAAAA AAAAAAAAAA AAAAAAAAAA AAAAAAAAAA AAAAAAAAAA
                                                    Poly(A)
```

### Exemplary Construct Encoding a SARS-CoV-2 Spike Protein

Structure m₂^{7,3′-O}Gppp(m₁^{2′-O})ApG)-hAg-Kozak-S1S2-PP-FI-A30L70
Encoded antigen Viral spike protein (S1S2 protein) of the SARS CoV-2 (S1S2 full-length protein, sequence variant)

### Exemplary Nucleotide Sequence Encoding a SARS-CoV-2 Spike Protein

Nucleotide sequence is shown with individual sequence elements as indicated in bold letters. In addition, the sequence of the translated protein is shown in italic letters below the coding nucleotide sequence (*=stop codon).

```
                10 20 30 40 50 53
AGAAUAAACU AGUAUUCUUC UGGUCCCCAC AGACUCAGAG AGAACCCGCC ACC
                                                    hAg-Kozak
                63 73 83 93 103 113
AUGUUCGUGU UCCUGGUGCU GCUGCCUCUG GUGUCCAGCC AGUGUGUGAA CCUGACCACC
    M F V F L V L L P L V S S Q C V N L T T
                                                    S protein
              123 133 143 153 163 173
AGAACACAGC UGCCUCCAGC CUACACCAAC AGCUUUACCA GAGGCGUGUA CUACCCCGAC
    R T Q L P P A Y T N S F T R G V Y Y P D
                                                    S protein
              183 193 203 213 223 233
AAGGUGUUCA GAUCCAGCGU GCUGCACUCU ACCCAGGACC UGUUCCUGCC UUUCUUCAGC
    K V F R S S V L H S T Q D L F L P F F S
                                                    S protein
              243 253 263 273 283 293
AACGUGACCU GGUUCCACGC CAUCCACGUG UCCGGCACCA AUGGCACCAA GAGAUUCGAC
    N V T W F H A I H V S G T N G T K R F D
                                                    S protein
              303 313 323 333 343 353
AACCCCGUGC UGCCCUUCAA CGACGGGGUG UACUUUGCCA GCACCGAGAA GUCCAACAUC
    N P V L P F N D G V Y F A S T E K S N I
                                                    S protein
              363 373 383 393 403 413
AUCAGAGGCU GGAUCUUCGG CACCACACUG GACAGCAAGA CCCAGAGCCU GCUGAUCGUG
    I R G W I F G T T L D S K T Q S L L I V
                                                    S protein
              423 433 443 453 463 473
AACAACGCCA CCAACGUGGU CAUCAAAGUG UGCGAGUUCC AGUUCUGCAA CGACCCCUUC
    N N A T N V V I K V C E F Q F C N D P F
                                                    S protein
              483 493 503 513 523 533
CUGGGCGUCU ACUACCACAA GAACAACAAG AGCUGGAUGG AAAGCGAGUU CCGGGUGUAC
    L G V Y Y H K N N K S W M E S E F R V Y
                                                    S protein
              543 553 563 573 583 593
AGCAGCGCCA ACAACUGCAC CUUCGAGUAC GUGUCCCAGC CUUUCCUGAU GGACCUGGAA
    S S A N N C T F E Y V S Q P F L M D L E
                                                    S protein
              603 613 623 633 643 653
GGCAAGCAGG GCAACUUCAA GAACCUGCGC GAGUUCGUGU UUAAGAACAU CGACGGCUAC
    G K Q G N F K N L R E F V F K N I D G Y
                                                    S protein
              663 673 683 693 703 713
UUCAAGAUCU ACAGCAAGCA CACCCCUAUC AACCUCGUGC GGGAUCUGCC UCAGGGCUUC
    F K I Y S K H T P I N L V R D L P Q G F
                                                    S protein
              723 733 743 753 763 773
UCUGCUCUGG AACCCCUGGU GGAUCUGCCC AUCGGCAUCA ACAUCACCCG GUUUCAGACA
    S A L E P L V D L P I G I N I T R F Q T
                                                    S protein
              783 793 803 813 823 833
CUGCUGGCCC UGCACAGAAG CUACCUGACA CCUGGCGAUA GCAGCAGCGG AUGGACAGCU
    L L A L H R S Y L T P G D S S S G W T A
                                                    S protein
              843 853 863 873 883 893
GGUGCCGCCG CUUACUAUGU GGGCUACCUG CAGCCUAGAA CCUUCCUGCU GAAGUACAAC
    G A A A Y Y V G Y L Q P R T F L L K Y N
                                                    S protein
              903 913 923 933 943 953
GAGAACGGCA CCAUCACCGA CGCCGUGGAU UGUGCUCUGG AUCCUCUGAG CGAGACAAAG
    E N G T I T D A V D C A L D P L S E T K
                                                    S protein
              963 973 983 993 1003 1013
UGCACCCUGA AGUCCUUCAC CGUGGAAAAG GGCAUCUACC AGACCAGCAA CUUCCGGGUG
    C T L K S F T V E K G I Y Q T S N F R V
                                                    S protein
            1023 1033 1043 1053 1063 1073
CAGCCCACCG AAUCCAUCGU GCGGUUCCCC AAUAUCACCA AUCUGUGCCC CUUCGGCGAG
    Q P T E S I V R F P N I T N L C P F G E
                                                    S protein
            1083 1093 1103 1113 1123 1133
GUGUUCAAUG CCACCAGAUU CGCCUCUGUG UACGCCUGGA ACCGGAAGCG GAUCAGCAAU
    V F N A T R F A S V Y A W N R K R I S N
                                                    S protein
            1143 1153 1163 1173 1183 1193
UGCGUGGCCG ACUACUCCGU GCUGUACAAC UCCGCCAGCU UCAGCACCUU CAAGUGCUAC
    C V A D Y S V L Y N S A S F S T F K C Y
                                                    S protein
            1203 1213 1223 1233 1243 1253
GGCGUGUCCC CUACCAAGCU GAACGACCUG UGCUUCACAA ACGUGUACGC CGACAGCUUC
    G V S P T K L N D L C F T N V Y A D S F
                                                    S protein
            1263 1273 1283 1293 1303 1313
GUGAUCCGGG GAGAUGAAGU GCGGCAGAUU GCCCCUGGAC AGACAGGCAA GAUCGCCGAC
    V I R G D E V R Q I A P G Q T G K I A D
                                                    S protein
            1323 1333 1343 1353 1363 1373
UACAACUACA AGCUGCCCGA CGACUUCACC GGCUGUGUGA UUGCCUGGAA CAGCAACAAC
    Y N Y K L P D D F T G C V I A W N S N N
                                                    S protein
            1383 1393 1403 1413 1423 1433
CUGGACUCCA AAGUCGGCGG CAACUACAAU UACCUGUACC GGCUGUUCCG GAAGUCCAAU
    L D S K V G G N Y N Y L Y R L F R K S N
                                                    S protein
            1443 1453 1463 1473 1483 1493
CUGAAGCCCU UCGAGCGGGA CAUCUCCACC GAGAUCUAUC AGGCCGGCAG CACCCCUUGU
    L K P F E R D I S T E I Y Q A G S T P C
                                                    S protein
            1503 1513 1523 1533 1543 1553
AACGGCGUGG AAGGCUUCAA CUGCUACUUC CCACUGCAGU CCUACGGCUU UCAGCCCACA
    N G V E G F N C Y F P L Q S Y G F Q P T
                                                    S protein
            1563 1573 1583 1593 1603 1613
AAUGGCGUGG GCUAUCAGCC CUACAGAGUG GUGGUGCUGA GCUUCGAACU GCUGCAUGCC
    N G V G Y Q P Y R V V V L S F E L L H A
                                                    S protein
            1623 1633 1643 1653 1663 1673
CCUGCCACAG UGUGCGGCCC UAAGAAAAGC ACCAAUCUCG UGAAGAACAA AUGCGUGAAC
    P A T V C G P K K S T N L V K N K C V N
                                                    S protein
            1683 1693 1703 1713 1723 1733
UUCAACUUCA ACGGCCUGAC CGGCACCGGC GUGCUGACAG AGAGCAACAA GAAGUUCCUG
    F N F N G L T G T G V L T E S N K K F L
                                                    S protein
            1743 1753 1763 1773 1783 1793
CCAUUCCAGC AGUUUGGCCG GGAUAUCGCC GAUACCACAG ACGCCGUUAG AGAUCCCCAG
    P F Q Q F G R D I A D T T D A V R D P Q
                                                    S protein
            1803 1813 1823 1833 1843 1853
ACACUGGAAA UCCUGGACAU CACCCCUUGC AGCUUCGGCG GAGUGUCUGU GAUCACCCCU
    T L E I L D I T P C S F G G V S V I T P
                                                    S protein
            1863 1873 1883 1893 1903 1913
GGCACCAACA CCAGCAAUCA GGUGGCAGUG CUGUACCAGG ACGUGAACUG UACCGAAGUG
    G T N T S N Q V A V L Y Q D V N C T E V
                                                    S protein
            1923 1933 1943 1953 1963 1973
CCCGUGGCCA UUCACGCCGA UCAGCUGACA CCUACAUGGC GGGUGUACUC CACCGGCAGC
    P V A I H A D Q L T P T W R V Y S T G S
                                                    S protein
            1983 1993 2003 2013 2023 2033
AAUGUGUUUC AGACCAGAGC CGGCUGUCUG AUCGGAGCCG AGCACGUGAA CAAUAGCUAC
    N V F Q T R A G C L I G A E H V N N S Y
                                                    S protein
            2043 2053 2063 2073 2083 2093
GAGUGCGACA UCCCCAUCGG CGCUGGAAUC UGCGCCAGCU ACCAGACACA GACAAACAGC
    E C D I P I G A G I C A S Y Q T Q T N S
                                                    S protein
            2103 2113 2123 2133 2143 2153
CCUCGGAGAG CCAGAAGCGU GGCCAGCCAG AGCAUCAUUG CCUACACAAU GUCUCUGGGC
    P R R A R S V A S Q S I I A Y T M S L G
                                                    S protein
            2163 2173 2183 2193 2203 2213
GCCGAGAACA GCGUGGCCUA CUCCAACAAC UCUAUCGCUA UCCCCACCAA CUUCACCAUC
    A E N S V A Y S N N S I A I P T N F T I
                                                    S protein
            2223 2233 2243 2253 2263 2273
AGCGUGACCA CAGAGAUCCU GCCUGUGUCC AUGACCAAGA CCAGCGUGGA CUGCACCAUG
    S V T T E I L P V S M T K T S V D C T M
                                                    S protein
            2283 2293 2303 2313 2323 2333
UACAUCUGCG GCGAUUCCAC CGAGUGCUCC AACCUGCUGC UGCAGUACGG CAGCUUCUGC
    Y I C G D S T E C S N L L L Q Y G S F C
                                                    S protein
            2343 2353 2363 2373 2383 2393
ACCCAGCUGA AUAGAGCCCU GACAGGGAUC GCCGUGGAAC AGGACAAGAA CACCCAAGAG
    T Q L N R A L T G I A V E Q D K N T Q E
                                                    S protein
            2403 2413 2423 2433 2443 2453
GUGUUCGCCC AAGUGAAGCA GAUCUACAAG ACCCCUCCUA UCAAGGACUU CGGCGGCUUC
    V F A Q V K Q I Y K T P P I K D F G G F
                                                    S protein
            2463 2473 2483 2493 2503 2513
AAUUUCAGCC AGAUUCUGCC CGAUCCUAGC AAGCCCAGCA AGCGGAGCUU CAUCGAGGAC
    N F S Q I L P D P S K P S K R S F I E D
                                                    S protein
            2523 2533 2543 2553 2563 2573
CUGCUGUUCA ACAAAGUGAC ACUGGCCGAC GCCGGCUUCA UCAAGCAGUA UGGCGAUUGU
    L L F N K V T L A D A G F I K Q Y G D C
                                                    S protein
            2583 2593 2603 2613 2623 2633
CUGGGCGACA UUGCCGCCAG GGAUCUGAUU UGCGCCCAGA AGUUUAACGG ACUGACAGUG
    L G D I A A R D L I C A Q K F N G L T V
                                                    S protein
            2643 2653 2663 2673 2683 2693
CUGCCUCCUC UGCUGACCGA UGAGAUGAUC GCCCAGUACA CAUCUGCCCU GCUGGCCGGC
    L P P L L T D E M I A Q Y T S A L L A G
                                                    S protein
            2703 2713 2723 2733 2743 2753
ACAAUCACAA GCGGCUGGAC AUUUGGAGCA GGCGCCGCUC UGCAGAUCCC CUUUGCUAUG
    T I T S G W T F G A G A A L Q I P F A M
                                                    S protein
            2763 2773 2783 2793 2803 2813
CAGAUGGCCU ACCGGUUCAA CGGCAUCGGA GUGACCCAGA AUGUGCUGUA CGAGAACCAG
    Q M A Y R F N G I G V T Q N V L Y E N Q
                                                    S protein
            2823 2833 2843 2853 2863 2873
AAGCUGAUCG CCAACCAGUU CAACAGCGCC AUCGGCAAGA UCCAGGACAG CCUGAGCAGC
    K L I A N Q F N S A I G K I Q D S L S S
                                                    S protein
            2883 2893 2903 2913 2923 2933
ACAGCAAGCG CCCUGGGAAA GCUGCAGGAC GUGGUCAACC AGAAUGCCCA GGCACUGAAC
    T A S A L G K L Q D V V N Q N A Q A L N
                                                    S protein
            2943 2953 2963 2973 2983 2993
ACCCUGGUCA AGCAGCUGUC CUCCAACUUC GGCGCCAUCA GCUCUGUGCU GAACGAUAUC
    T L V K Q L S S N F G A I S S V L N D I
                                                    S protein
            3003 3013 3023 3033 3043 3053
CUGAGCAGAC UGGACCCUCC UGAGGCCGAG GUGCAGAUCG ACAGACUGAU CACAGGCAGA
    L S R L D P P E A E V Q I D R L I T G R
                                                    S protein
            3063 3073 3083 3093 3103 3113
CUGCAGAGCC UCCAGACAUA CGUGACCCAG CAGCUGAUCA GAGCCGCCGA GAUUAGAGCC
    L Q S L Q T Y V T Q Q L I R A A E I R A
                                                    S protein
            3123 3133 3143 3153 3163 3173
UCUGCCAAUC UGGCCGCCAC CAAGAUGUCU GAGUGUGUGC UGGGCCAGAG CAAGAGAGUG
    S A N L A A T K M S E C V L G Q S K R V
                                                    S protein
            3183 3193 3203 3213 3223 3233
GACUUUUGCG GCAAGGGCUA CCACCUGAUG AGCUUCCCUC AGUCUGCCCC UCACGGCGUG
    D F C G K G Y H L M S F P Q S A P H G V
                                                    S protein
            3243 3253 3263 3273 3283 3293
GUGUUUCUGC ACGUGACAUA UGUGCCCGCU CAAGAGAAGA AUUUCACCAC CGCUCCAGCC
    V F L H V T Y V P A Q E K N F T T A P A
                                                    S protein
            3303 3313 3323 3333 3343 3353
AUCUGCCACG ACGGCAAAGC CCACUUUCCU AGAGAAGGCG UGUUCGUGUC CAACGGCACC
    I C H D G K A H F P R E G V F V S N G T
                                                    S protein
            3363 3373 3383 3393 3403 3413
CAUUGGUUCG UGACACAGCG GAACUUCUAC GAGCCCCAGA UCAUCACCAC CGACAACACC
    H W F V T Q R N F Y E P Q I I T T D N T
                                                    S protein
            3423 3433 3443 3453 3463 3473
UUCGUGUCUG GCAACUGCGA CGUCGUGAUC GGCAUUGUGA ACAAUACCGU GUACGACCCU
    F V S G N C D V V I G I V N N T V Y D P
                                                    S protein
            3483 3493 3503 3513 3523 3533
CUGCAGCCCG AGCUGGACAG CUUCAAAGAG GAACUGGACA AGUACUUUAA GAACCACACA
    L Q P E L D S F K E E L D K Y F K N H T
                                                    S protein
            3543 3553 3563 3573 3583 3593
AGCCCCGACG UGGACCUGGG CGAUAUCAGC GGAAUCAAUG CCAGCGUCGU GAACAUCCAG
    S P D V D L G D I S G I N A S V V N I Q
                                                    S protein
            3603 3613 3623 3633 3643 3653
AAAGAGAUCG ACCGGCUGAA CGAGGUGGCC AAGAAUCUGA ACGAGAGCCU GAUCGACCUG
    K E I D R L N E V A K N L N E S L I D L
                                                    S protein
            3663 3673 3683 3693 3703 3713
CAAGAACUGG GGAAGUACGA GCAGUACAUC AAGUGGCCCU GGUACAUCUG GCUGGGCUUU
    Q E L G K Y E Q Y I K W P W Y I W L G F
                                                    S protein
            3723 3733 3743 3753 3763 3773
AUCGCCGGAC UGAUUGCCAU CGUGAUGGUC ACAAUCAUGC UGUGUUGCAU GACCAGCUGC
    I A G L I A I V M V T I M L C C M T S C
                                                    S protein
            3783 3793 3803 3813 3823 3833
UGUAGCUGCC UGAAGGGCUG UUGUAGCUGU GGCAGCUGCU GCAAGUUCGA CGAGGACGAU
    C S C L K G C C S C G S C C K F D E D D
                                                    S protein
            3843 3853 3863 3873 3878
UCUGAGCCCG UGCUGAAGGG CGUGAAACUG CACUACACAU GAUGA
    S E P V L K G V K L H Y T * *
                                                    S protein
            3888 3898 3908 3918 3928 3938
CUCGAGCUGG UACUGCAUGC ACGCAAUGCU AGCUGCCCCU UUCCCGUCCU GGGUACCCCG
                                                    FI element
            3948 3958 3968 3978 3988 3998
AGUCUCCCCC GACCUCGGGU CCCAGGUAUG CUCCCACCUC CACCUGCCCC ACUCACCACC
                                                    FI element
            4008 4018 4028 4038 4048 4058
UCUGCUAGUU CCAGACACCU CCCAAGCACG CAGCAAUGCA GCUCAAAACG CUUAGCCUAG
                                                    FI element
            4068 4078 4088 4098 4108 4118
CCACACCCCC ACGGGAAACA GCAGUGAUUA ACCUUUAGCA AUAAACGAAA GUUUAACUAA
                                                    FI element
            4128 4138 4148 4158 4168 4173
GCUAUACUAA CCCCAGGGUU GGUCAAUUUC GUGCCAGCCA CACCCUGGAG CUAGC
                                                    FI element
            4183 4193 4203 4213 4223 4233
AAAAAAAAAA AAAAAAAAAA AAAAAAAAAA GCAUAUGACU AAAAAAAAAA AAAAAAAAAA
                                                    Poly(A)
            4243 4253 4263 4273 4283
AAAAAAAAAA AAAAAAAAAA AAAAAAAAAA AAAAAAAAAA AAAAAAAAAA
                                                    Poly(A)
```

In some embodiments, a pharmaceutical composition comprising one or more polynucleotides encoding a polypeptide encoded by a string construct may be co-administered with another vaccine for treating a viral disease. In some embodiments, such a viral disease is an infectious respiratory disease (e.g., but not limited to flu (influenza), respiratory syncytial virus, etc.). In some embodiments, such a viral disease is COVID. In some embodiments, a pharmaceutical composition comprising one or more polynucleotides encoding a polypeptide encoded by a string construct may be co-administered with a coronavirus vaccine (e.g., RNA-based, protein-based, DNA-based, etc.). In some embodiments, a pharmaceutical composition comprising one or more polynucleotides encoding a polypeptide encoded by a string construct may be co-administered with a RNA-based coronavirus vaccine. In some embodiments, the pharmaceutical composition comprising a string construct may be co-administered, for example, with an antibody, such as a neutralizing antibody that can bind to a SARS CoV-2 protein, e.g., orflab polyprotein, orfla polyprotein, surface glycoprotein(S), nucleocapsid phosphoprotein (N), ORF3a protein, membrane glycoprotein (M), ORF7a protein, ORF8 protein, envelope protein (E), ORF6 protein, ORF7b protein or ORF10 protein. In some embodiments, the pharmaceutical composition may be co-administered with an antibody directed to the SARS spike protein. In some embodiments, the pharmaceutical composition comprising one or more polynucleotides encoding a polypeptide encoded by a string construct may be administered before, after or simultaneously with a therapeutic regime comprising another vaccine described above.

In some aspects, a polypeptide encoded by a string construct, especially comprising SARS COV-2 nucleocapsid protein epitopes are designed to boost the immunogenicity and immune memory against the virus. Certain of the present day vaccines in trial comprise vaccines directed to the viral spike proteins, that are likely to confer an immunogenic response, but do not appear to elicit or promote a T cell response. In some embodiments, vaccines comprising a string construct or a polypeptide encoded by a string construct described herein can elicit or promote a T cell response and/or elicit or promote a lasting immunological memory. In some embodiments, a vaccine against SARS CoV-2 may be accompanied by one or more string vaccine compositions described herein, e.g., as part of an administration regimen, such as for a boost after priming. In some embodiments, a vaccine against SARS CoV-2 may be mRNA-based, viral vector-based (e.g., replicating and/or non-replicating), DNA-based, protein-based (e.g., protein subunit and/or virus like particles), and/or inactivated/attenuated virus-based. In some embodiments, such a vaccine is directed to a spike protein or an immunogenic fragment thereof. In some embodiments, such a SARS CoV-2 vaccine may be or comprise an mRNA-based vaccine against SARs-CoV-2, e.g., in some embodiments a mRNA-based vaccine (mRNA-1273) developed by Moderna that encodes a prefusion stabilized form of SARS CoV-2 Spike protein. In some embodiments, such a SARS CoV-2 vaccine may be or comprise a viral vector based vaccine against SARS-CoV-2, e.g., in some embodiments an adenovirus vaccine vector-based vaccine (AZD1222) developed by AstraZeneca that is made from a virus (e.g., ChAdOx1), which is a weakened version of an adenovirus, and encodes a SARS CoV-2 spike protein.

### Pharmaceutical Composition Comprising String Constructs or a Polypeptide Encoded by a String Construct

In one aspect, a pharmaceutical composition comprising the string vaccines may be administered to a patient alone or in combination with other drugs or vaccines.

In some embodiments, the pharmaceutical composition comprising the string vaccine may be administered before, simultaneously or after an initial administration of another vaccine or drug for SARS CoV-2 viral infection.

In some embodiments, the pharmaceutical composition comprising the string vaccine may be administered 7 days, 2 weeks, 3 weeks, 4 weeks, 5 weeks, 6 weeks, 7 weeks, 8 weeks, 9 weeks, 10 weeks, 11 weeks, 12 weeks, 14 weeks, 16 weeks, 18 weeks, or 20 weeks or more before administering another vaccine or drug for SARS CoV-2 viral infection. The pharmaceutical composition comprising the string vaccine may be administered prophylactically, or as a preventive vaccine, similar to for example, the flu vaccine at the onset of annual flu season.

In some embodiments, the pharmaceutical composition comprising the string vaccine may be administered 7 days, 2 weeks, 3 weeks, 4 weeks, 5 weeks, 6 weeks, 7 weeks, 8 weeks, 9 weeks, 10 weeks, 11 weeks, 12 weeks, 14 weeks, 16 weeks, 18 weeks, or 20 weeks or more after the administration of a vaccine or a drug for 2019 SARS CoV-2 viral infection. In some embodiments, the pharmaceutical composition comprising the string vaccine may be administered 3 months after another 2019 SARS-CoV-2 vaccine therapy. In some embodiments, the pharmaceutical composition comprising the string vaccine may be administered 6 months after another 2019 SARS-CoV-2 vaccine therapy. In some embodiments, the pharmaceutical composition comprising the string vaccine may be administered 8 months after another 2019 SARS-CoV-2 vaccine therapy. In some embodiments, the pharmaceutical composition comprising the string vaccine may be administered 9 months after another 2019 SARS-CoV-2 vaccine therapy. In some embodiments, the pharmaceutical composition comprising the string vaccine may be administered 10 months after another SARS-CoV-2 vaccine therapy. In some embodiments, the pharmaceutical composition comprising the string vaccine may be administered 12 months after another 2019 SARS-CoV-2 vaccine therapy.

In some embodiments, the pharmaceutical composition comprising the string vaccine may be administered once every 2 days, 7 days, 2 weeks, 3 weeks, 4 weeks, 5 weeks, 6 weeks, 7 weeks, 8 weeks, 9 weeks, 10 weeks, 12 weeks or more. In some embodiments, the pharmaceutical composition comprising a string vaccine (e.g., as described herein) may be administered once every 20 days, 21 days, 22 days, 23 days, 24 days, 25 days, 26 days, 27 days, 28 days, 29 days, 30 days, or more. In some embodiments, the pharmaceutical composition comprising a string vaccine (e.g., as described herein) may be administered once every 3 months, 4 months, 5 months, 6 months, 7 months, 8 months, 9 months, 10 months, 11 months, 12 months, or longer. In some embodiments, a subject may be administered at least two doses of the pharmaceutical composition comprising a string vaccine (e.g., as described herein), and the at least two doses of the pharmaceutical composition comprising the string vaccine may be administered at an interval of 20 days. In some embodiments, two such doses may be administered at an interval of 21 days. In some embodiments, two such doses may be administered at an interval of 22 days. In some embodiments, two such doses may be administered at an interval of 23 days. In some embodiments, two such doses may be administered at an interval of 24 days. In some embodiments, two such doses may be administered at an interval of 25 days. In some embodiments, two such doses may be administered at an interval of 26 days. In some embodiments, two such doses may be administered at an interval of 27 days. In some embodiments, two such doses may be administered at an interval of 28 days. In some embodiments, the pharmaceutical composition comprising the string vaccine may be administered as a boost (or maintenance) once every 6 months, or once 8 month or once every 12 months after an initial phase of priming dose comprising more frequent dosing. In some embodiments the priming dose may comprise 2, 3, 4, 5, 6, 7, 8, 9, 10 or more doses.

In some embodiments the string vaccine compositions may be used at a dose between 1-1000 microgram per dose per person. In some embodiments, the string vaccine composition may be administered at a dose of 1-600 micrograms per dose, per person. In some embodiments, the string vaccine composition may be administered at a dose of 1-500 micrograms per dose, per person. In some embodiments, the string vaccine composition may be administered at a dose of 1-400 micrograms per dose, per person. In some embodiments, the string vaccine composition may be administered at a dose of 1-300 micrograms per dose, per person. In some embodiments, the string vaccine composition may be administered at a dose of 1-200 micrograms per dose, per person. In some embodiments, the string vaccine composition may be administered at a dose of 10-300 micrograms per dose, per person. In some embodiments, the string vaccine composition may be administered at a dose of 10-200 micrograms per dose, per person. In some embodiments, the string vaccine composition may be administered at a dose of 10-100 micrograms per dose, per person. In some embodiments, the string vaccine composition may be administered at a dose of 10 micrograms per dose, per person. In some embodiments, the string vaccine composition may be administered at a dose of 20 micrograms per dose, per person. In some embodiments, the string vaccine composition may be administered at a dose of 30 micrograms per dose, per person. In some embodiments, the string vaccine composition may be administered at a dose of 40 micrograms per dose, per person. In some embodiments, the string vaccine composition may be administered at a dose of 50 micrograms per dose, per person. In some embodiments, the string vaccine composition may be administered at a dose of 60 micrograms per dose, per person. In some embodiments, the string vaccine composition may be administered at a dose of 70 micrograms per dose, per person. In some embodiments, the string vaccine composition may be administered at a dose of 80 micrograms per dose, per person. In some embodiments, the string vaccine composition may be administered at a dose of 90 micrograms per dose, per person. In some embodiments, the string vaccine composition may be administered at a dose of 100 micrograms per dose, per person. In some embodiments, the string vaccine composition may be administered at a dose of 120 micrograms per dose, per person. In some embodiments, the string vaccine composition may be administered at a dose of 150 micrograms per dose, per person.

In some embodiments where a string vaccine composition (e.g., as described herein) is administered in combination with a BNT RNA vaccine composition, e.g., a composition comprising an RNA (e.g., mRNA) encoding a viral spike protein (e.g., a SARS CoV-2 S protein or an immunogenic fragment thereof (e.g., RBD)), which in some embodiments may be encapsulated in a lipid nanoparticle, such a BNT RNA vaccine composition may be administered at a dose ranging from 0.1 micrograms to 100 micrograms, 1 to 60 micrograms, 3 to 50 micrograms, 3-30 micrograms, or 10-30 micrograms. In some embodiments, such a BNT RNA vaccine composition may be administered at a dose of 1, 2, 3, 4, 5, 6, 7, 8, 9, 10, 15, 20, 25, 30 micrograms or more. In some embodiments, a BNT RNA vaccine comprises an RNA (e.g., mRNA) construct encoding a SARS CoV-2 S protein, which can have a structure represented as m₂^{7,3′-O}Gppp(m₁^{2′-O})ApG)-hAg-Kozak-S1S2-PP-FI-A30L70.

In some embodiments, a BNT RNA vaccine composition (e.g., as described herein) to be administered in combination with a string vaccine composition (e.g., as described herein) may comprise an initial dose, e.g., the priming dose; and a follow up dose, e.g., a booster dose. In some embodiments, the priming dose and the booster dose are administered at an interval of 20 days. In some embodiments, such BNT RNA vaccine composition doses may be administered at an interval of 21 days. In some embodiments, such BNT RNA vaccine composition doses may be administered at an interval of 22 days. In some embodiments, such BNT RNA vaccine composition doses may be administered at an interval of 23 days. In some embodiments, such BNT RNA vaccine composition may be administered at an interval of 24 days. In some embodiments, such BNT RNA vaccine composition doses may be administered at an interval of 25 days. In some embodiments, such BNT RNA vaccine composition doses may be administered at an interval of 26 days. In some embodiments, such BNT RNA vaccine composition doses may be administered at an interval of 27 days. In some embodiments, such BNT RNA vaccine composition doses may be administered at an interval of 28 days. In some embodiments, such BNT RNA vaccine composition may be administered at an interval of longer than 28 days, e.g., including, e.g., every 1 month, 2 months, 3 months, 4 months, 5 months, 6 months, 7 months, 8 months, 9 months, 10 months, 11 months, 12 months, or longer.

In some embodiments, a BNT RNA vaccine composition (e.g., as described herein) to be administered in combination with a string vaccine composition (e.g., as described herein) may comprise a modified RNA encoding a viral spike protein (e.g., a SARS CoV-2 S protein or an immunogenic fragment thereof (e.g., RBD)), in which one or more uridine nucleotide residues is replaced with a modified uridine nucleotide (e.g., 1-methylpseudouridine). In some embodiments, at least 10%, or at least 20%, or at least 30%, or at least 40%, or at least 50%, or at least 60% or at least 70%, or at least 80% or at least 90% of the U nucleotides of the structure are replaced by a modified uridine nucleotide (e.g., 1-methylpseudouridine). In some embodiments, 100% of the U nucleotides of the structure are replaced by a modified uridine nucleotide (e.g., 1-methylpseudouridine).

In some embodiments the vaccine comprising a nucleotide sequence encoding a spike protein may be co-administered with an RNA vaccine comprising a string construct. In some embodiments, the vaccine comprising a nucleotide sequence encoding a spike protein vaccine is administered as an initial dose, followed by an RNA vaccine comprising a string construct comprising sequences encoding 2, 3, 4, 5, 6, 7, 8, 9, 10 or more epitopes from 2 or more viral proteins, as a follow up dose, a maintenance dose, a second dose, a third dose or as one or more booster doses. In some embodiments, a vaccine comprising a nucleotide sequence encoding a spike protein is administered as an initial dose, followed by an RNA vaccine comprising a string construct comprising sequences encoding 2, 3, 4, 5, 6, 7, 8, 9, 10 or more epitopes from 2 or more viral proteins as a follow up dose, a maintenance dose, a second dose, a third dose or as one or more booster doses. In some embodiments, an RNA vaccine comprising a string construct comprising sequences encoding 2, 3, 4, 5, 6, 7, 8, 9, 10 or more epitopes from 2 or more viral proteins is administered to a subject as an initial dose, followed by a vaccine comprising a nucleotide sequence encoding a spike protein as a follow up dose, a maintenance dose, a second dose, a third dose or as one or more booster doses.

In some embodiments, the pharmaceutical composition comprising a string construct may comprise a coformulation vaccine. In some embodiments, the coformulation vaccine composition may comprise a first string vaccine at a first concentration, and a second string vaccine at a second concentration, and third string vaccine at a third concentration and so on. In some embodiments, a first string vaccine may comprise a vaccine comprising a nucleotide sequence encoding a spike protein.

In some embodiments, a coformulation composition may comprise a first polynucleotide composition, comprising a nucleotide vaccine encoding a spike protein or fragment thereof. In some embodiments, the coformulation may comprise a first nucleotide sequence, having a structure m₂^{7,3′-O}Gppp(m₁^{2′-O})ApG)-hAg-Kozak-S1S2-PP-FI-A30L70, as described above. In some embodiments, the coformulation may comprise a second composition comprising a RS C5, RS C6, RS C7, and RS C8 or a combination thereof. In some embodiments, the coformulation may comprise a second composition comprising a RS C1, RS C2, RS C3, and RS C4 or a combination thereof. In some embodiments, a first nucleotide sequence, having a structure m₂^{7,3′-O}Gppp(m₁^{2′-O})ApG)-hAg-Kozak-S1S2-PP-FI-A30L70 and a second nucleotide sequence having a RS C1, RS C2, RS C3, RS C4, RS C5, RS C6, RS C7, or RS C8 may be present at a ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 or 1:1. In some embodiments, the coformulation may comprise a second composition comprising a RS C1, RS C2, RS C3, and RS C4 or a combination thereof. In some embodiments, a first nucleotide sequence, having a structure m₂^{7,3′-O}Gppp(m₁^{2′-O})ApG)-hAg-Kozak-S1S2-PP-FI-A30L70 and a second nucleotide sequence having a RS C1, RS C2, RS C3, RS C4, RS C5, RS C6, RS C7, or RS C8 may be present at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9, 9:8, 1:11, 1:12, 1:13, 1:14, 1:15, 1:16, 1:17, 1:18, 1:19 or 1:20.

In some embodiments, polynucleotides described herein (e.g., a polynucleotide encoding a viral spike protein and/or a polynucleotide encoding a peptide (e.g., comprising an epitope sequence as described herein) may be encapsulated in lipid nanoparticles. In some embodiments, such lipid nanoparticle may comprise one or more cationic or ionizable lipids. In some embodiments, such lipid nanoparticle may optionally comprise neutral lipids (e.g., phospholipids and/or sterols such as, e.g., cholesterol), and/or polymer-conjugated lipids, such as PEGylated lipids.

In some embodiments, a pharmaceutical composition comprising subject specific T cells may be generated ex vivo, where the subject specific T cell population may be responsive to at least one of the epitopes in Table 1A, Table 1B, Table 1C, Table 2Ai, Table 2Aii, Table 2B, Table 9, Table 10, Table 11, Table 12, Table 14A, Table 14B, Table 14C, Table 15 or Table 16, or any antigen disclosed in the specification corresponding to a viral antigen. In one embodiment, PBMC from a subject may be isolated (e.g., from a leukapheresis sample), and incubated in the presence of one or more epitopes that are disclosed in any one of the tables (Table 1A, Table 1B, Table 1C, Table 2Ai, Table 2Aii, Table 2B, Table 9, Table 10, Table 11, Table 12, Table 14A, Table 14B, Table 14C, Table 15 or Table 16) In some embodiments the antigen may be selected based on the MHC peptides present in the subject, such that the antigen peptides have high affinity and presentation prediction score in combination with the MHC, based on the peptide: MHC pairs disclosed in Table 1A, Table 1B, Table 1C, Table 2Ai, Table 2Aii, Table 2B, Table 9, Table 10, Table 11, Table 12, Table 14A, Table 14B, Table 15 or Table 16.

In some embodiments, a pharmaceutical composition comprises: (i) a peptide comprising an epitope sequence selected from: NYNYLYRLF; KWPWYIWLGF; QYIKWPWYI; LPFNDGVYF; QPTESIVRF; IPFAMQMAY; YLQPRTELL; and/or RLQSLQTYV; (ii) a polynucleotide encoding the peptide; (iii) a T cell receptor (TCR) or a T cell comprising the TCR, wherein the TCR binds to the epitope sequence in complex with a corresponding MHC class I or class II molecule; (iv) an antigen presenting cell comprising (i) or (ii); or (v) an antibody or B cell comprising the antibody, wherein the antibody binds to the epitope sequence.

In some embodiments, a pharmaceutical composition comprising T cells may be generated ex vivo wherein the T cells may be responsive to an epitope sequence comprising or consisting of NYNYLYRLF, wherein in some embodiments the subject expresses an MHC protein encoded by HLA-A*2402. In some embodiments, a pharmaceutical composition comprising subject specific T cells may be generated ex vivo wherein the T cells may be responsive to an epitope sequence comprising or consisting of, KYIKWPWYI, wherein in some embodiments the subject expresses an MHC protein encoded by HLA-A*2402. In some embodiments, a pharmaceutical composition comprising subject specific T cells may be generated ex vivo wherein the T cells may be responsive to an epitope sequence comprising or consisting of KWPWYIWLGF, wherein in some embodiments the subject expresses an MHC protein encoded by HLA-A*2402. In some embodiments, a pharmaceutical composition comprising subject specific T cells may be generated ex vivo wherein the T cells may be responsive to an epitope sequence comprising or consisting of QYIKWPWYI, wherein in some embodiments the subject expresses an MHC protein encoded by HLA-A*2402.

In some embodiments, a pharmaceutical composition comprising T cells may be generated ex vivo wherein the T cells may be responsive to an epitope sequence comprising or consisting of LPFNDGVYF, wherein in some embodiments the subject expresses an MHC protein encoded by HLA-B*3501. In some embodiments, a pharmaceutical composition comprising T cells may be generated ex vivo wherein the T cells may be responsive to an epitope sequence comprising or consisting of QPTESIVRF, wherein in some embodiments the subject expresses an MHC protein encoded by HLA-B*3501. In some embodiments, a pharmaceutical composition comprising T cells may be generated ex vivo wherein the T cells may be responsive to an epitope sequence comprising or consisting of, IPFAMQMAY, wherein in some embodiments the subject expresses an MHC protein encoded by HLA-B*3501.

In some embodiments, a pharmaceutical composition comprising T cells may be generated ex vivo wherein the T cells may be responsive to an epitope sequence comprising or consisting of YLQPRTFLL, wherein in some embodiments the subject expresses an MHC protein encoded by HLA-A*0201. In some embodiments, a pharmaceutical composition comprising T cells may be generated ex vivo wherein the T cells may be responsive to an epitope sequence comprising or consisting of RLQSLQTYV, wherein in some embodiments the subject expresses an MHC protein encoded by HLA-A*0201.

In some embodiment, a string vaccine may be formulated to be delivered in an aqueous solution systemically by injection to a subject. The string vaccine may comprise one or more polynucleotides, such as RNA, such as mRNA. In some embodiments the mRNA may be associated with one or more lipids. In some embodiments, the string vaccine may be co-formulated to comprise one or more strings, one or more spike mRNA vaccines and one or more strings comprising epitope sequences covering one or more of the other viral proteins, ORF1ab, nucleocapsid, membrane protein or a combination thereof. In some embodiments, the vaccine is formulated for systemic injection, such as intramuscular, subcutaneous, intravenous, intraocular.

In some embodiments the string mRNA is contacted to a cell population, comprising antigen presenting cells and T cells. In some embodiments, the string mRNA is electroporated in a cell, such as an APC. In some embodiments, T cells are generated as described elsewhere within the application, that are primed with APCs expressing one or more strings.

In some embodiments, the pharmaceutical composition comprises a CorVac 2.0 string, e.g., RS-C7 (C7). In some embodiments, C7 string mRNA is encapsulated in a lipid nanoparticle (LNP) in a single mRNA-LNP formulation. In some embodiments, a pharmaceutical composition comprises a lipid nanoparticle formulation that comprises a C7 string mRNA and one or more other LNPs encapsulating one, two, three or four other CorVac 2.0 string mRNAs, or more, such as a RS-C5 (C5) string, a RS-C6 (C6) string and a RS-C8 (C8) string. In some embodiments, a pharmaceutical composition comprises a lipid nanoparticle formulation that comprises a C7 string mRNA and one or more other LNPs formulations each encapsulating a different string mRNA, e.g. a CorVac 2.0 string. In one embodiment, the pharmaceutical composition comprises a mixture of different LNPs, comprising one, two, three or four other CorVac 2.0 string mRNAs, such as a C5 string in an LNP formulation, a C6 string in an LNP formulation, or a C8 string in a separate LNP formulation. In some embodiments, the LNPs are mixed in a specific ratio with respect to each other. In some embodiments, a C7 string-LNP and another string mRNA LNP may be mixed in a ratio (e.g., mass ratio) of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8 in a pharmaceutical composition. In some embodiments, the LNPs are mixed in a specific ratio with respect to each other. In some embodiments, a C7 string-LNP and another string mRNA LNP is mixed in a ratio (e.g., mass ratio) of 1:11, 1:12, 1:13, 1:14, 1:15, 1:16, 1:17, 1:18, 1:19 or 1:20 in a pharmaceutical composition. In some embodiments, a C7 string-LNP and another string mRNA LNP is mixed in a ratio (e.g., mass ratio) of 1:25, 1:30, 1:40, 1:50, 1:60, 1:70, 1:80, 1:90 or 1:100 in a pharmaceutical composition. In some embodiments, a C7 string-LNP and another string mRNA LNP is mixed in a ratio (e.g., mass ratio) of 1:0.1, 1:0.2, 1:0.3, 1:0.4, 1:0.5, 1:0.6, 1:0.7, 1:0.8, 1:0.9 or 1:1 in a pharmaceutical composition. In some embodiments, a C7 string-LNP and another string mRNA LNP is mixed in a ratio (e.g., mass ratio) of 1:0.01, 1:0.02, 1:0.03, 1:0.04, 1:0.05, 1:0.06, 1:0.07, 1:0.08, 1:0.09 in a pharmaceutical composition. In some embodiments, a pharmaceutical composition comprises a first CorVac 2.0 string mRNA and a second, a third and/or a fourth CorVac 2.0 string mRNA formulated in a single LNP. In some embodiments a pharmaceutical composition comprises a single LNP comprising one or more different CorVac 2.0 mRNA strings, of which one is a C7 string.

In some embodiments, provided herein is a pharmaceutical composition, comprising (i) a recombinant polynucleotide encoding a polypeptide comprising at least two of the following: a sequence comprising an epitope sequence from ORF1ab, a sequence comprising an epitope sequence from membrane glycoprotein (M), and a sequence comprising an epitope sequence from nucleocapsid phosphoprotein (N), wherein each epitope can be of the same or different pathogen (e.g., a virus); and (ii) a recombinant polynucleotide encoding a Spike protein of a virus or an immunogenic fragment thereof (e.g., receptor binding domain), or a variant thereof. In some embodiments, a Spike protein of a virus is 2019 SARS-CoV 2 spike protein (SARS-CoV-2 S protein) or a variant or fragment thereof.

In some embodiments, the recombinant polynucleotide encodes a SARS-CoV-2 S protein of SEO ID NO: Spike p, shown below.

(SEQ ID NO: Spike_p)
MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVIGIVNNTVYDPLQPELDSFKEELD
KYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCS
CGSCCKFDEDDSEPVLKGVKLHYT

In some embodiments, the recombinant polynucleotide in (ii) encodes a Spike protein of a virus or an immunogenic fragment thereof (e.g., receptor binding domain), or a variant thereof that comprises one or more mutations (e.g., insertions, substitions, or deletions) that increase Spike protein expression and/or stability of a prefusion conformation. In some embodiments, a Spike protein of a virus is 2019 SARS-CoV 2 spike protein (SARS-CoV-2 S protein) or a variant or fragment thereof. In some embodiments, full length spike(S) protein is modified in such a way that the prototypical prefusion conformation is stabilized. Certain mutations that stabilize a prefusion confirmation are known in the art, e.g., as disclosed in WO 2021243122 A2; Hsieh, Ching-Lin, et al. (“Structure-based design of prefusion-stabilized SARS-CoV-2 spikes,” Science 369.6510 (2020): 1501-1505); WO2022/067010 A1; WO2018081318A1; WO2021163365A1; and WO2021213924A1, the contents of each which are incorporated by reference herein in their entirety. In some embodiments, a SARS-CoV-2 S protein may be stabilized by introducing one or more proline mutations. In some embodiments, a Spike protein of a virus (e.g., in some embodiments SARS-CoV-2 S protein) comprises a proline substitution at positions corresponding to residues 986 and/or 987 of SEQ ID NO: Spike_p. In some embodiments, a Spike protein of a virus (e.g., in some embodiments SARS-CoV-2 S protein) comprises proline substitutions at positions corresponding to one or more of residues 817, 892, 899, and 942 of SEQ ID NO: Spike_p. In some embodiments, a Spike protein of a virus (e.g., in some embodiments a SARS-CoV-2 S protein) comprises a proline substitution at positions corresponding to each of residues 817, 892, 899, and 942 of SEQ ID NO: Spike_p. In some embodiments, a Spike protein of a virus (e.g., a SARS-CoV-2 S protein) comprises a proline substitution at positions corresponding to each of residues 817, 892, 899, 942, 986, and 987 of a SARS-CoV-2 S protein of SEQ ID NO: Spike_p.

In some embodiments, the recombinant polynucleotide in (ii) encodes a Spike protein of a virus that comprises one or more mutations in a furin cleavage site. In some embodiments, a Spike protein of a virus is 2019 SARS-CoV 2 spike protein (SARS-CoV-2 S protein) or a variant or fragment thereof (e.g., in some embodiments, a Spike protein comprises a mutation at a position corresponding to residues 682-685 of SEQ ID NO: Spike_p). In some embodiments, the mutation in the furin cleavage site prevents cleavage by a furin protease (e.g., a human furin protease). In some embodiments, an encoded SARS-CoV-2 S protein described herein comprises a furin mutation disclosed in WO2021163365 or WO2021243122 (e.g., a GSAS mutation), the contents of both of which are incorporated by reference herein in their entirety.

In some embodiments, the recombinant polynucleotide (ii) encodes a receptor binding domain (RBD) of a virus (e.g., a 2019 SARS-CoV 2 spike protein RBD) or a variant or fragment thereof. In some embodiments, the recombinant polynucleotide (ii) polypeptide comprises a sequence that corresponds to the RBD, and further comprises a trimerization domain (e.g., a trimerization domain as disclosed herein, such as a fibritin domain). In some embodiments an RBD comprises a signaling domain (e.g., a signaling domain as disclosed herein). In some embodiments an RBD comprises a transmembrane domain (e.g., a transmembrane domain as disclosed herein). In some embodiments, an RBD comprises a signaling domain and a trimerization domain. In some embodiments, an RBD comprises a signaling domain, a trimerization domain, and transmembrane domain. In some embodiments, the encoded polypeptide comprises a sequence that comprises two receptor binding domains. In some embodiments, the encoded polypeptide comprises two receptor binding domains in tandem in an amino acid chain, e.g., as disclosed in Dai, Lianpan, et al. “A universal design of betacoronavirus vaccines against COVID-19, MERS, and SARS,” Cell 182.3 (2020): 722-733, the contents of which are incorporated by reference herein in their entirety. In some embodiments, each of the RBDs have the same amino acid sequence. In some embodiments, each of the RBDs in tandem have different amino acid sequences (e.g., in some embodiments, the two RBDs may each separately comprise one or mutations that are characteristic of a different SARS-CoV-2 strain or variant.

In some embodiments, the recombinant polypeptide (ii) encodes a SARS-CoV-2 S protein comprising one or more mutations characteristic of a SARS-CoV-2 variant or a fragment thereof. In some embodiments, a SARS-CoV-2 variant refers to a variant of concern (e.g., a newly identified variant of concern, e.g., a newly identified variant of concern identified in a relevant jurisidiction). In some embodiments, a SARS-CoV-2 variant refers to a variant that is prevalent and/or rapidly spreading in a relevant jurisdiction. Such variants can be identified based on publicly available data (e.g., data provided in the GISAID Initiative database: https://www.gisaid.org, and/or data provided by the World Health Organization WHO (e.g., as provided at https://www.who.int/activities/tracking-SARS-CoV-2-variants). In some embodiments, a SARS-CoV-2 variant refers to a variant disclosed herein, e.g., a variant listed in Table A. In some embodiments, a SARS-CoV-2 variant refers to an Omicron variant, e.g., a BA.1, BA.2, BA.12.1, or a BA.4/5 variant listed in Table C.

TABLE C
Exemplary Omicron Variants

| Variant | Description |
|---|---|
| BA.1 | A67V, Δ69-70, T95I, G142D, Δ143-145, Δ211, L212I, ins214EPE, |
| | G339D, S371L, S373P, S375F, K417N, N440K, G446S, |
| | S477N,T478K, E484A, Q493R, G496S, Q498R, N501Y, Y505H, |
| | T547K, D614G, H655Y, N679K, P681H, N764K, D796Y, N856K, |
| | Q954H, N969K, and L981F |
| BA.2 | T19I, Δ24-26, A27S, G142D, V213G, G339D, S371F, S373P, |
| | S375F, T376A, D405N, R408S, K417N, N440K, S477N, T478K, |
| | E484A, Q493R, Q498R, N501Y, Y505H, D614G, H655Y, N679K, |
| | P681H, N764K, D796Y, Q954H, and N969K |
| BA.2.12.1 | T19I, Δ24-26, A27S, G142D, V213G, G339D, S371F, S373P, |
| | S375F, T376A, D405N, R408S, K417N, N440K, L452Q, S477N, |
| | T478K, E484A, Q493R, Q498R, N501Y, Y505H, D614G, H655Y, |
| | N679K, P681H, S704L, N764K, D796Y, Q954H, N969K |
| BA.4/BA.5: | T19I, Δ24-26, A27S, Δ69/70, G142D, V213G, G339D, S371F, |
| | S373P, S375F, T376A, D405N, K417N, N440K, L452R, S477N, |
| | T478K, E484A, F486V, Q498R, N501Y, Y505H, D614G, H655Y, |
| | N679K, P681H, N764K, D796Y, Q954H, and N969K |
| BA 2.75: | T19I, Δ24-26, A27S, G142D, K147E, W152R, F157L, I210V, |
| | V213G, G257S, G339H, N354D, S371F, S373P, S375F, T376A, |
| | D405N, R408S, K417N, N440K, G446S, N460K, S477N, T478K, |
| | E484A, Q498R, N501Y, Y505H D614G, H655Y, N679K, P681H, |
| | N764K, D796Y, Q954H, and N969K |

In some embodiments, provided herein is a pharmaceutical composition, comprising (i) a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and (ii) a recombinant polynucleotide encoding a 2019 SARS-CoV 2 spike protein or a variant or fragment thereof; wherein the (ii) a recombinant polynucleotide encoding a 2019 SARS-CoV 2 spike protein or a variant or fragment thereof; wherein the ratio (e.g., mass ratio) of (i):(ii) is greater than 20:1 or less than 1:20. Ratios disclosed herein, when used to describe the relative amount of two or more RNAs in a composition or administered to a subject, refer to the amount of one RNA in a composition or administered to a subject relative to one or more additional RNAs in the same composition or administered to the same subject. In some embodiments, a ratio refers to a mass ratio (e.g., the mass of one RNA in a composition or administered to a subject relative to the mass of one or more RNAs in the same composition or administered to the same subject). In some embodiments, a ratio refers to a molar ratio (e.g., the number of moles of one RNA in a composition or administered to a subject relative to the number of moles of one or more RNAs in the same composition or administered to the same subject). In some embodiments, the recombinant polynucleotide in (i) and the recombinant polynucleotide in (ii) is mRNA. In some embodiments, the recombinant polynucleotide in (i) and the recombinant polynucleotide of (ii) are encapsulated in separate LNPs. In some embodiments, the recombinant polynucleotide in (i) and the recombinant polynucleotide of (ii) are encapsulated in the same LNPs. In some embodiments, the LNP comprising the recombinant polynucleotide (i) and the LNP comprising recombinant polynucleotide (ii) are present in a ratio (e.g., mass ratio) of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20.

In some embodiments, a pharmaceutical composition comprises one or more LNPs, at least one comprising a CorVac 2.0 string mRNA, and at least one comprising a BNT162b2 string mRNA. In some embodiments, a pharmaceutical composition comprises a C7 string mRNA encapsulated in an LNP, and a BNT162b2 string mRNA encapsulated in an LNP. In some embodiments, a pharmaceutical composition comprises a CorVac 2.0 string mRNA and a BNT162b2 string mRNA formulated in a single LNP. In some embodiments a pharmaceutical composition comprises a CorVac 2.0 string mRNA, e.g. a C7 string mRNA encapsulated in a first LNP formulation, and a BNT162b2 string mRNA encapsulated in a second LNP, wherein the two LNPs are mixed in the pharmaceutical composition to be present in a certain ratio with respect to each other. In some embodiments, the CorVac 2.0 string mRNA-LNP and a BNT162b2 string mRNA-LNP are mixed in a ratio (e.g., mass ratio) of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 1:9.5, 1:8.5, 1:7.5, 1:6.5, 1:5.5, 1:4.5, 1:3.5, 1:2.5, 1:1.5, 2.5:1, 3.5:1, 4.5:1, 5.5:1, 6.5:1, 7.5:1, 8.5:1, 9.5:1, 2:9, 2:8, 2:7, 2:6, 2:5, 2:4, 2:3, 3:2, 4:2, 5:2, 6:2, 7:2, 8:2, 9:2, 3:8, 3:7, 3:5, 3:4, 4:3, 5:3, 7:3, 8:3, 4:9, 4:7, 4:5, 5:4, 7:4, 9:4, 5:9, 5:8, 5:7, 5:6, 6:5, 7:5, 8:5, 9:5, 6:7, 7:6, 7:8, 8:7, 8:9 or 9:8 in a pharmaceutical composition. In some embodiments, the CorVac 2.0 string mRNA-LNP and a BNT162b2 string mRNA-LNP are mixed in a ratio (e.g., mass ratio) of 1:11, 1:12, 1:13, 1:14, 1:15, 1:16, 1:17, 1:18, 1:19 or 1:20. In some embodiments, the CorVac 2.0 string mRNA-LNP and a BNT162b2 string mRNA-LNP are mixed in a ratio (e.g., mass ratio) of 1:25, 1:30, 1:40, 1:50, 1:60, 1:70, 1:80, 1:90 or 1:100 in a pharmaceutical composition. In some embodiments, the CorVac 2.0 string mRNA-LNP and a BNT162b2 string mRNA-LNP are mixed in a ratio (e.g., mass ratio) of 1:0.1, 1:0.2, 1:0.3, 1:0.4, 1:0.5, 1:0.6, 1:0.7, 1:0.8, 1:0.9 or 1:1 in a pharmaceutical composition. In some embodiments, the CorVac 2.0 string mRNA-LNP and a BNT162b2 string mRNA-LNP are mixed in a ratio (e.g., mass ratio) of 1:0.01, 1:0.02, 1:0.03, 1:0.04, 1:0.05, 1:0.06, 1:0.07, 1:0.08, 1:0.09 in a pharmaceutical composition. In some embodiments, the different mRNA strings are formulated in different LNP compositions, and administered separately. In some embodiments, each mRNA LNP is stored in a separate vial, and are administered in separate times, at a time interval of 5-30 minutes, or 1 hour, or 2 hours, or 4 hours, or 6 hours, or 8 hours, or 12 hours, or 16 hours, or 20 hours, or 24 hours, or 36 hours, or 48 hours, or 72 hours or 96 hours or more. In some embodiments, each mRNA LNP is stored in a separate vial, and are administered in separate times, at a time interval of 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days or 30 days. In some embodiments, each mRNA LNP are administered in separate times, at a time interval of between 1 month and 3 months, between 1 month and 6 months, between 2 months and 6 months, between 1 month and 1 year.

In some embodiments, a first pharmaceutical composition comprising a first LNP comprising a first string mRNA, e.g., a BNT162b2 mRNA is administered in one arm of the subject, and a second pharmaceutical composition comprising a second LNP comprising a second string mRNA, e.g., a CorVac 2.0 mRNA is administered in the other arm of the subject, at an interval of 5-30 minutes, or 1 hour, or 2 hours, or 4 hours, or 6 hours, or 8 hours, or 12 hours, or 16 hours, or 20 hours, or 24 hours, or 36 hours, or 48 hours, or 72 hours or 96 hours or more.

In some embodiments, two or more pharmaceutical compositions comprising one, two or more LNP compositions, each encapsulating a different mRNA string, are mixed at the patient's bedside prior to administration. Instructions for mixing can be adequately provided in vial label, on the kit containing the vials, or in instruction sheet provided in the kit. In some embodiments, the mixing is performed at a given ratio. In some embodiments, the LNPs are provided in the pharmaceutical composition such that mixing the pharmaceutical compositions at a ratio of 1:1 results in the respective LNP compositions to be present in the required ratio suitable for administration.

In some embodiments, any vaccine composition comprising the spike mRNA vaccine or a string vaccine or a string vaccine in combination with other therapeutics may be administered to a selected patient group, depending on the age, health condition, gender, medical histories, ethnicity in relation to disease propensity and outcome and so forth. In some embodiments, patient population may be categorized as high risk based on age, health condition, gender, medical histories, ethnicity in relation to disease propensity and outcome and so forth. A therapeutic comprising a string vaccine or a string vaccine in combination with a second therapeutic may be administered to a patient population only if the patient population has been categorized as high risk. Conversely, a therapeutic comprising a string vaccine or a string vaccine in combination with a second therapeutic may be administered to a patient population only if the patient population has been categorized as low risk. In some embodiments, the vaccine composition, alone or in combination may be to patients of 19-55 years of age. In some embodiments, the vaccine composition, alone or in combination may be to patients of 12-65 years of age. In some embodiments, the vaccine composition, alone or in combination may be to patients of 12-35 years of age. In some embodiments, the vaccine composition, alone or in combination may be to patients of 19-35 years of age. In some embodiments, the vaccine composition, alone or in combination may be to patients of 35-55 years of age. In some embodiments, the vaccine composition, alone or in combination may be to patients of 40-65 years of age. In some embodiments, the vaccine composition, alone or in combination may be to patients of 65-85 years of age. In some embodiments, the vaccine composition, alone or in combination may be to patients of age 12 or younger. In some embodiments, the vaccine composition, alone or in combination may be to patients of age 10 or younger. In some embodiments, the vaccine composition, alone or in combination may be to adolescent populations (e.g., individuals approximately 12 to approximately 17 years of age). In some embodiments, the vaccine composition, alone or in combination may be to a pediatric population. In various embodiments, the pediatric population comprises or consists of subjects under 18 years, e.g., 5 to less than 18 years of age, 12 to less than 18 years of age, 16 to less than 18 years of age, 12 to less than 16 years of age, or 5 to less than 12 years of age. In various embodiments, the pediatric population comprises or consists of subjects under 5 years, e.g., 2 to less than 5 years of age, 12 to less than 24 months of age, 7 to less than 12 months of age, or less than 6 months of age.

In some embodiments, a therapeutic comprising a string vaccine or a string vaccine in combination with a second therapeutic may be administered to a patient who has one or more comorbidities, such as a chronic illness, e.g., cancer, diabetes, kidney disease or CFTR. In some embodiments, a therapeutic comprising a string vaccine or a string vaccine in combination with a second therapeutic may not be administered to a patient who has one or more comorbidities, such as a chronic illnesses. In some embodiments, a therapeutic comprising a string vaccine or a string vaccine in combination with a second therapeutic may be administered to subjects whose profession and/or environmental exposure may dramatically increase their risk of getting SARS CoV-2 infection (including, e.g., but not limited to mass transportation, prisoners, grocery store workers, residents in long-term care facilities, butchers or other meat processing workers, healthcare workers, and/or first responders, e.g., emergency responders). In some embodiments, a therapeutic comprising a string vaccine or a string vaccine in combination with a second therapeutic may be administered to healthcare workers and/or first responders, e.g., emergency responders.

In some embodiments, a therapeutic comprising a string vaccine or a string vaccine in combination with a second therapeutic may be administered to those with a history of smoking or vaping (e.g., within 6 months, 12 months or more, including a history of chronic smoking or vaping). In some embodiments, a therapeutic comprising a string vaccine or a string vaccine in combination with a second therapeutic may be administered to certain ethnic groups that have been determined to be more susceptible to SARS CoV-2 infection. In some embodiments, a therapeutic comprising a string vaccine or a string vaccine in combination with a second therapeutic may be administered to certain populations with a blood type that may have been determined to more susceptible to SARS CoV-2 infection. In some embodiments, a therapeutic comprising a string vaccine or a string vaccine in combination with a second therapeutic may be administered to immunocompromised subjects (e.g., those with HIV/AIDS; those with an age-related decline in immunity, immunosenescence, multifactoral immunodeficiency, or elderly or older alduts with an age-related immuodeficiency; cancer and transplant patients who are taking certain immunosuppressive drugs; autoimmune diseases or other physiological conditions expected to warrant immunosuppressive therapy (e.g., within 3 months, within 6 months, or more); and those with inherited diseases that affect the immune system (e.g., congenital agammaglobulinemia, congenital IgA deficiency)). In some embodiments, a therapeutic comprising a string vaccine or a string vaccine in combination with a second therapeutic may be administered to those with an infectious disease. For example, in some embodiments, a therapeutic comprising a string vaccine or a string vaccine in combination with a second therapeutic may be administered to those infected with human immunodeficiency virus (HIV) and/or a hepatitis virus (e.g., HBV, HCV). In some embodiments, a therapeutic comprising a string vaccine or a string vaccine in combination with a second therapeutic may be administered to those with underlying medical conditions. Examples of such underlying medical conditions may include, but are not limited to hypertension, cardiovascular disease, diabetes, chronic respiratory disease, e.g., chronic pulmonary disease, asthma, etc., cancer, and other chronic diseases such as, e.g., lupus, rheumatoid arthritis, chronic liver diseases, chronic kidney diseases (e.g., Stage 3 or worse such as in some embodiments as characterized by a glomerular filtration rate (GFR) of less than 60 mL/min/1.73 m2). In some embodiments, a therapeutic comprising a string vaccine or a string vaccine in combination with a second therapeutic may be administered to overweight or obese subjects, e.g., specifically including those with a body mass index (BMI) above about 30 kg/m2. In some embodiments, a therapeutic comprising a string vaccine or a string vaccine in combination with a second therapeutic may be administered to subjects who have prior diagnosis of COVID-19 or evidence of current or prior SARS CoV-2 infection, e.g., based on serology or nasal swab.

In some embodiments, a therapeutic comprising a string vaccine or a string vaccine in combination with a second therapeutic may be administered to a subject with a B cell immunodeficiency. In some embodiments, the subject has a reduced ability to produce an antibody response to an antigen.

In some embodiments, the subject with a B cell immunodeficiency has a reduced ability to produce an antibody response to a vaccination. In some embodiments, the subject with a B cell immunodeficiency has a reduced ability to produce an anti-spike protein antibody response. In some embodiments, the subject with a B cell immunodeficiency can produce a T cell response or does not have a reduced ability to produce a T cell response.

In some embodiments, the subject with a B cell immunodeficiency is an organ transplant recipient. In some embodiments, the subject with a B cell immunodeficiency received an organ transplant less than 1 year, less than 6 months or less than 3 months after the pharmaceutical composition is administered. In some embodiments, the subject with a B cell immunodeficiency is expected to receive an organ transplant less than 1 year, less than 6 months or less than 3 months prior to the pharmaceutical composition being administered.

In some embodiments, the subject with a B cell immunodeficiency has a cancer. In some embodiments, wherein the cancer is a B cell cancer. In some embodiments, wherein the B cell cancer is a B cell lymphoma or a B cell leukemia.

In some embodiments, the subject with a B cell immunodeficiency has an autoimmune disease or condition. In some embodiments, in the autoimmune disease or condition is Addison disease, Anti-NMDA receptor encephalitis, antisynthetase syndrome, Aplastic anemia, autoimmune anemias, Autoimmune hemolytic anemia, Autoimmune pancreatitis, Behcet's Disease, bullous skin disorders, Celiac disease-sprue (gluten-sensitive enteropathy), chronic fatigue syndrome, Chronic inflammatory demyelinating polyneuropathy, chronic lymphocytic leukemia, Crohn's disease, Dermatomyositis, Devic's disease, Erythroblastopenia, Evans syndrome, Focal segmental glomerulosclerosis, Granulomatosis with polyangiitis, Graves disease, Graves' ophthalmopathy, Guillain-Barre syndrome, Hashimoto thyroiditis, idiopathic thrombocytopeniaurpura (ITP), IgA nephropathy, IgA-mediated autoimmune diseases, IgG4-related disease, Inflammatory bowel disease, Juvenile idiopathic arthritis, Multiple sclerosis, Myasthenia gravis, myeloma, non-Hodgkin's lymphoma, Opsoclonus myoclonus syndrome (OMS), Pemphigoid, Pemphigus, pemphigus vulgaris, Pernicious anemia, polymyositis, Psoriasis, pure red cell aplasia, Reactive arthritis, Rheumatoid arthritis, Sarcoidosis, scleroderma, Sjögren syndrome, Systemic lupus erythematosus, Thrombocytopenia purpura, Thrombotic thrombocytopeniarpura, Type I diabetes, Ulcerative colitis, Vasculitis (e.g., vasculitis associated with anti-neutrophil cytoplasmic antibody) and Vitiligo

In some embodiments, the subject with a B cell immunodeficiency does not have has congenital agammaglobulinemia or congenital IgA deficiency.

In some embodiments, the subject with a B cell immunodeficiency is receiving an immunosuppressive agent or has received an immunosuppressive agent less than 1 year, less than 6 months or less than 3 months prior to the administering of the pharmaceutical composition. In some embodiments, the immunosuppressive agent is abatacept (e.g. ORENCIA), abrilumab, acalabrutinib, adalimumab, adrenocorticotropic hormone, agatolimod sodium, AJM300, aldesleukin, alefacept, alemtuzumab, alisertib, alvespimycin hydrochloride, alvocidib, ambrisentan (e.g. LETAIRIS), aminocamptothecin, amiselimod, anakinra, andecaliximab, andrographolides (a botanical medicinal herb also known as IB-MS), anifrolumab, antithymocyte Ig, apatinib, apelisib, asparaginase, atacicept, atezolizumab, avelumab, azacitidine, azathioprine, bafetinib, baminercept, baricitinib, basiliximab, becatecarin, begelomab, belatacept, belimumab, bemcentinib, bendamustine, bendamustine (e.g. bendamustine hydrochloride), betalutin with lilotomab, bevacizumab, BIIB033, BIIB059, BIIB061, bimekizumab, binimetinib, bleomycin, blinatumomab, BNZ-1, bortezomib (e.g. VELCADE), brentuximab vedotin, bryostatin 1, bucillamine, buparlisib, busulfan, canakinumab, capecitabine, carboplatin, carfilzomib, carmustine, cediranib maleate, cemiplimab, ceralifimod, cerdulatinib, certolizumab (e.g. certolizumab pegol), cetuximab, chidamide, chlorambucil, CHS-131, cilengitide, cirmtuzumab, cisplatin, cladribine, clazakizumab, clemastine, clioquinol, corticosteroids, cyclophosphamide, cyclosporine, cytarabine, cytotoxic chemotherapy, daclizumab, dalfampridine (e.g. AMPYRA), daprolizumab pegol, daratumumab, dasatinib, defactinib, defibrotide, denosumab, dexamethasone, diacerein, dimethyl fumarate, dinaciclib, diroximel fumarate (e.g. VUMERITY), doxorubicin, doxorubicin (e.g. doxorubicin hydrochloride), durvalumab, duvelisib, duvortuxizumab, eculizumab (e.g. SOLIRIS), efalizumab, eftilagimod alpha, EK-12 (a neuropeptide combination of metenkefalin and tridecactide), elezanumab, elotuzumab (e.g. EMPLICITI), encorafenib, enfuvirtida (e.g. FUZEON), entinostat, entospletinib, enzastaurin, epacadostat, epirubicin, epratuzumab, eritoran tetrasodium, etanercept, etoposide, etrolizumab, everolimus, evobrutinib, filgotinib, fingolimod (e.g. fingolimod hydrochloride), firategrast, fludarabine, fluorouracil, fontolizumab, forodesine hydrochloride, fostamatinib, galunisertib, ganetespib, ganitumab, gemcitabine, gemtuzumab ozogamicin, gerilimzumab, glasdegib, glassia, glatiramer acetate, glembatumumab vedotin, glesatinib, golimumab (e.g. SIMPONI), guadecitabine, hydrocortisone, hydroxychloroquine sulfate, hydroxyurea, ibritumomab tiuxetan, ibrutinib, ibudilast, idarubicin, idebenone, idelalisib, ifosfamide, iguratimod, imatinib, imexon, IMU-838, infliximab, inotuzumab ozogamicin, interferon alfa-2, interferon beta-la, interferon beta-1b, interferon gamma-1, ipilimumab, irofulven, isatuximab, ispinesib, itacitinib, ixazomib, lapatinib, laquinimod, laromustine, ld-aminopterin, leflunomide, lenalidomide, lenvatinib, letrozole (e.g. FEMARA), levamisole, levocabastine, lipoic acid, lirilumab, lonafarnib, lumiliximab, maraviroc (e.g. SELZENTRY), masitinib, mavrilimumab, melphalan, mercaptopurine, methotrexate, methoxsalen, methylprednisone, milatuzumab, mitoxantrone, mizoribine, mocetinostat, monalizumab, mosunetuzumab, motesanib diphosphate, moxetumomab pasudotox, muromonab-CD3, mycophenolate mofetil (e.g. mycophenolate mofetil hydrochloride), mycophenolic acid, namilumab, natalizumab, navitoclax, neihulizumab, nerispirdine, neurovax, niraparib, nivolumab, obatoclax mesylate, obinutuzumab, oblimersen sodium, ocrelizumab, ofatumumab, olokizumab, opicinumab, oprelvekin, osimertinib, otelixizumab, oxaliplatin, oxcarbazepine, ozanimod, paclitaxel, pacritinib, palifermin, panobinostat, pazopanib, peficitinib, pegfilgrastim (e.g. NEULASTA), peginterferon beta-la, pegsunercept (peg stnf-ri), pembrolizumab, pemetrexed, penclomedine, pentostatin, perifosine, pevonedistat, pexidartinib, picoplatin, pidilizumab, pivanex, pixantrone, pleneva, plovamer acetate, polatuzumab vedotin, pomalidomide, ponatinib, ponesimod, prednisone/prednisolone, pyroxamide, R-411, ravulizimab-cwvz (e.g. (ULTOMIRIS), recombinant il-12, relatlimab, rhigf-1, rhigm22, rigosertib, rilonacept, ritonavir (e.g. NORVIR), rituximab, ruxolitinib, SAR442168/PRN2246, sarilumab, secukinumab, selumetinib, simvastatin, sintilimab, siplizumab, siponimod (e.g. MAYZENT), sirolimus (rapamycin), sirukumab, sitravatinib, sonidegib, sorafenib, sotrastaurin acetate, sunitinib, sunphenon epigallocatechin-gallate, tabalumab, tacrolimus (e.g. tacrolimus anhydrous), talabostat mesylate, talacotuzumab, tanespimycin, tegafur/gimeracil/oteracil, tenalisib, terameprocol, teriflunomide, thalidomide, thiarabine, thiotepa, tipifarnib, tirabrutinib, tislelizumab, tivozanib, tocilizumab, tofacitinib, TR-14035, tregalizumab, tremelimumab, treosulfan, ublituximab, umbralisib, upadacitinib, urelumab, ustekinumab, varlilumab, vatelizumab, vedolizumab, veliparib, veltuzumab, venetoclax, vinblastine, vincristine, vinorelbine ditartrate, visilizumab, vismodegib, vistusertib, voriconazole (e.g. VFEND), vorinostat, vosaroxin, ziv-aflibercept or any combination thereof. In some embodiments, the immunosuppressive agent is 2B3-201, 3PRGD2, 4SC-202, 506U78, 6,8-bis(benzylthio) octanoic acid, 68Ga-BNOTA-PRGD2, 852A, 89Zr-DFO-CZP, ABBV-257, ABL001, ABP 501, ABP 710, ABP 798, ABT-122, ABT-199, ABT-263, ABT-348, ABT-494, ABT-555, ABT-874, ABX-1431 HCl, ACP-196, ACP-319, ACT-128800, ACY-1215, AD 452, Ad-P53, ADCT-301, ADCT-402, ADL5859, ADS-5102, AFX-2, AGEN1884, AGEN2034, AGS67E, AlN457, AK106-001616, ALD518, ALKS 8700, ALT-803, ALT-803, ALX-0061, ALXN1007, ALXN6000, AMD3100, AMG 108, AMG 319, AMG 357, AMG 570, AMG 592, AMG 714, AMG 719, AMG 827, AMP-110, AP1903, APL A12, APO866, APX005M, AQ4N, AR-42, ARN-6039, ARQ 531, ARRY-371797, ARRY-382, ARRY-438162, ART-102, ART621, ASK8007, ASN002, ASP015K, ASP1707, ASP2408, ASP2409, ASP5094, AT-101, AT7519M, AT9283, ATA188, ATN-103, ATX-MS-1467, AVL-292, AVP-923, AZD4573, AZD5672, AZD5991, AZD6244, AZD6738, AZD9056, AZD9150, AZD9567, AZD9668, B-701, BAF312, BAY1830839, BBI608, BCD-054, BCD-055, BCD-063, BCD-089, BCD-100, BCD-132, BCD-145, BEZ235, BG00012, BG9924, BGB-3111, BGB-A333, BGG492, BHT-3009, BI 655064, BI 695500, BI 695501, BI 836826, BI-1206, BIBR 796 BS, BIIB017, BIIB023, BIIB057, BIIB061, BIIL 284 BS, BLZ945, BMMNC, BMN 673, BMS-247550, BMS-582949, BMS-817399, BMS-936558, BMS-936564, BMS-945429, BMS-986104, BMS-986142, BMS-986156, BMS-986195, BMS-986205, BMS-986213, BMS-986226, BMS-986251, BNC105P, BOW015, BP1001, BT061, BTT-1023, C105, CAL-101, CAM-3001, CAT-8015, CB-839, CBL0137, CC-1088, CC-115, CC-122, CC-292, CC100, CCI-779, CCX 354-C, CDKI AT7519, CDP323, CDP6038, CDP870, CDX-1127, CDX-301, CE-224535, CF101, CFZ533, CGP 77116, CH-1504, CH-4051, CHR-5154, CHS-0214, CK-2017357, CLAG-M, CLR 131, CMAB008, CMP-001, CNF2024 (BIIB021), CNM-Au8, CNTO 1275, CNTO 136, CNTO 148, CNTO 6785, CP-195543, CP-461, CpG 7909, CPI-1205, CR6086, CRx-102, CS-0777, CS1002, CT-011, CT-1530, CT-P10, CV301, CX-3543, DAC-HYP, DCDT2980S, DI-B4, DPA-714 FDG, DS-3032b, DT2219ARL, DTRM-505, DTRM-555, DTRMWXHS-12, DWP422, E6011, E7449, EK-12, ELND002, ENIA11, EOC202, ETBX-011, F8IL10, FBTA05, FEDAA1106 (BAY85-8101), FGF401, FKB327, FPA008, FR104, FS118, FTY720, G¹⁰⁰, GCS-100, GDC-0199, GDC-0853, GEH120714, GLPG0259, GLPG0634, GNbAC1, GNKG168, GP2013, GP2015, GRN163L, GS-1101, GS-5745, GS-9219, GS-9820, GS-9876, GS-9901, GSK1223249, GSK1827771, GSK2018682, GSK21110183, GSK239512, GSK2618960, GSK2831781, GSK2982772, GSK3117391, GSK3152314A, GSK3196165, GSK3358699, GSK706769, GW-1000-02, GW274150, GW406381, GW856553, GZ402668, HCD122, HE3286, HL2351, HL237, hLL1-DOX (IMMU-115), HLX01, HM71224, HMPL-523, HSC835, HZT-501, ICP-022, IDEC-C2B8, ILV-094, IMGN529, IMMU-114, IMO-2125, INCAGN02385, INCB018424, INCB028050, INCB039110, INCB047986, INCMGA00012, INNO-406, INT131, INT230-6, INVAC-1, IPI-145, IPX056, ISF35, ISIS 104838, ITF2357, JCARH125, JHL1101, JNJ 38518168, JNJ-39758979, JNJ-40346527, JNJ-63723283, JS001, JTE-051, JTX-2011, KB003, KD025, KPT-330, KW-2449, KW-2478, KX2-391, L-778123, LAG525, LAM-002A, LBEC0101, LBH589, LFB-R603, LMB-2, LX3305, LY2127399, LY2189102, LY2439821, LY3009104, LY3090106, LY3300054, LY3321367, LY3337641, M2951, M7824, M923, MBG453, MBP8298, MBS2320, MD1003, MDG013, MDV9300, MDX-1100, MDX-1342, MDX-1411, ME-401, MEDI-522, MEDI-538, MEDI-551, MEDI4920, MGA012, MGCD0103, MGD007, MIS416, MK-0873, MK-4280, MK-4827, MK-8457, MK-8808, MK0359, MK0457, MK0752, MK0782, MK0812, MK2206, MLN1202, MLTA3698A, MM-093, MN-122, MN-166, monoclonal antibody M-T412, monoclonal antibody mono-dgA-RFB4, MOR00208, MOR103, MORAb-022, MP-435, MP470, MRC375, MRG-106, MS-533, MSB11022, MSC2490484A, MT-1303, MT-3724, MTIG7192A, MTRX1011A, NBI-5788, NC-503, NI-0101, NI-071, NIS793, NKTR-214, NNC 0141-0000-0100, NNC 0151-0000-0000, NNC0109-0012, NNC0114-0000-0005, NNC0114-0006, NNC0142-0002, NNC0215-0384, NNC109-0012, NOX-A12, NT-KO-003, NU100, OMB157, OMP-313M32, ON01910 Na, ONO-2506PO, ONO-4641, ONTAK, OPB 31121, OSI-461, OTS167IV, P1446A-05, PBF-509, PBR06, PCI 32765, PCI-24781, PD 0360324, PDA001, PDR001, PF-04171327, PF-04236921, PF-04308515, PF-04629991, PF-05280586, PF-06342674, PF-06410293, PF-06438179, PF-06650833, PF-06651600, PF-06835375, PG-760564, PH-797804, PLA-695, PLX3397, PLX5622, POL6326, PRO131921, PRO283698, PRTX-100, PS-341, PTL201, R(+) XK469, R788, RAD001, RC18, REGN1979, REGN3767, REGN2810, REGN4659, RFT5-SMPT-dgA, RG2077, RGB-03, RGI-2001, RHB-104, RNS60, RO5045337, RO7123520, Rob 803, RPC1063, RWJ-445380, S 55746, SAIT101, SAN-300, SAR245409, SB-681323, SB683699, SBI-087, SC12267 (4SC-101), SCH 727965, SCIO-469, SD-101, SG2000, SGN-40, SHC014748M, SHR-1210, SHR0302, SHR1020, SJG-136, SKI-O-703, SMP-114, SNS-032, SNS-062, SNX-5422, SPARC1103 I, SPC2996, SSR150106, STA 5326 mesylate, Sunpharma1505, SyB L-0501, Sym022, Sym023, SYN060, T-614, T0001, TA-650, TAB08, TAK-715, TAK-783, TAK-901, TGR-1202, TH-302, TL011, TMI-005, TMP001, TNFa Kinoid, TP-0903, TRU-015, TRU-016, TSR-022, TSR-033, TSR-042, TXA127, VAY736, VP-16, VSN16R, VX-509, VX-702, VX-745, VX15/2503, XCEL-MC-ALPHA, XL228, XL844, XmAb13676, XmAb5574, XOMA 052, YRA-1909, Z102, ZEN003365, or any combination thereof. In some embodiments, the immunosuppressive agent is A2aR antagonist, Akt inhibitor, anti CD20, Anti-amyloidotic (AA) Agent, anti-CD37 protein therapeutic, anti-CTLA4 mAb, Anti-CXCR4, anti-huCD40 mAb, anti-LAG3 mAb, anti-PD-1 mAb, anti-PD-L1 agent, anti-PD-L1 agent, anti-PD-L1 mAb, anti-TGFb mAb, anti-TIGIT mAb, anti-TIM-3 mAb, Aurora kinase inhibitor, Bcl-2 Inhibitor, bifunctional fusion protein targeting TGFb and PD-L1, bispecific anti-PD-1 and anti-LAG3 mAb, CD1d ligand, CD40 agonist, Complement C5a inhibitor, CSF1R inhibitor, EZH2 inhibitor, FGFR3 inhibitor, FGFR4 inhibitor, FGFrR3 inhibitor, glucocorticoid-induced tumor necrosis factor receptor-related gene [GITR] agonist, glutaminase inhibitor, Human monoclonal antibody against IL-12, ICOS agonist, IDO1 inhibitor, IL2 mutein, IL2 receptor agonist, MEK inhibitor, multitargeted receptor tyrosine kinase inhibitor, neutrophil elastase inhibitor, Notch Inhibitor, p38 MAPK inhibitor, PD-1 inhibitor, recombinant human Flt3L, ROCK inhibitor, selective sphingosine-1-phosphate receptor modulator, Src kinase inhibitor, TLR4 agonist, TLR9 agonist, or any combination thereof. In some embodiments, the immunosuppressive agent is a Complement C5a inhibitor, a CD40 agonist, a p38 inhibitor, a CSF1R inhibitor, a MEK inhibitor, a neutrophil elastase inhibitor, FGFrR3 inhibitor, anti-LAG3 mAb, Anti-CXCR, glucocorticoid-induced tumor necrosis factor receptor-related gene [GITR] agonist, IDO1 inhibitor, ICOS agonist, glutaminase inhibitor, recombinant human Flt3L, TLR9 agonist, EZH2 inhibitor, anti-CTLA4 mAb, PD-1 inhibitor, PD-L1 inhibitor, anti-PD-L1 mAb, FGFR4 inhibitor, bispecific anti-PD-1 and anti-LAG3 mAb, TLR4 agonist, Bcl-2 Inhibitor, anti-LAG3 mAb, an inhibitor of a cell degradation pathways, or a proteasome inhibitor. In some embodiments, the immunosuppressive agent is adalimumab (e.g., HUMIRA), alemtuzumab (e.g., LEMTRADA), alemtuzumab (e.g., CAMPATH), azathioprine (e.g., IMURAN), belimumab (e.g., BENLYSTA), bevacizumab (e.g., AVASTIN), bortezomib (e.g., VELCADE), eculizumab (e.g., SOLIRIS), leflunomide, brentuximab vedotin (e.g., ADCETRIS), cetuximab (e.g., ERBITUX), cyclophosphamid, dimethyl fumarate (e.g., TECFIDERA), efalizumab (e.g., RAPTIVA), fingolimod (e.g., GILENYA), fludarabine (e.g., FLUDARA), fumaric acid, imatinib (e.g., GLEEVEC, GLIVEC), infliximab (e.g., REMICADE), methotrexate (e.g., TREXALL, RHEUMATREX), mycophenolate mofetil (e.g., CELLCEPT), natalizumab (e.g., TYSABRI), daclizumab (e.g., ZINBRYTA), rituximab (e.g., RITUXAN), vedolizumab (e.g., ENTYVIO), ruxolitinib (e.g., JAKAFI, JAKAVI), or ocrelizumab (e.g., Ocrevus). In some embodiments, the immunosuppressive agent is abatacept (e.g. ORENCIA), abrilumab, acalabrutinib, adalimumab, adrenocorticotropic hormone, agatolimod sodium, AJM300, aldesleukin, alefacept, alemtuzumab, alisertib, alvespimycin hydrochloride, alvocidib, ambrisentan (e.g. LETAIRIS), aminocamptothecin, amiselimod, anakinra, andecaliximab, andrographolides (a botanical medicinal herb also known as IB-MS), anifrolumab, antithymocyte Ig, apatinib, apelisib, asparaginase, atacicept, atezolizumab, avelumab, azacitidine, azathioprine, bafetinib, baminercept, baricitinib, basiliximab, becatecarin, begelomab, belatacept, belimumab, bemcentinib, bendamustine, bendamustine (e.g. bendamustine hydrochloride), betalutin with lilotomab, bevacizumab, BIIB033, BIIB059, BIIB061, bimekizumab, binimetinib, bleomycin, blinatumomab, BNZ-1, bortezomib (e.g. VELCADE), brentuximab vedotin, bryostatin 1, bucillamine, buparlisib, busulfan, canakinumab, capecitabine, carboplatin, carfilzomib, carmustine, cediranib maleate, cemiplimab, ceralifimod, cerdulatinib, certolizumab (e.g. certolizumab pegol), cetuximab, chidamide, chlorambucil, CHS-131, cilengitide, cirmtuzumab, cisplatin, cladribine, clazakizumab, clemastine, clioquinol, corticosteroids, cyclophosphamide, cyclosporine, cytarabine, cytotoxic chemotherapy, daclizumab, dalfampridine (e.g. AMPYRA), daprolizumab pegol, daratumumab, dasatinib, defactinib, defibrotide, denosumab, dexamethasone, diacerein, dimethyl fumarate, dinaciclib, diroximel fumarate (e.g. VUMERITY), doxorubicin, doxorubicin (e.g. doxorubicin hydrochloride), durvalumab, duvelisib, duvortuxizumab, eculizumab (e.g. SOLIRIS), efalizumab, eftilagimod alpha, EK-12 (a neuropeptide combination of metenkefalin and tridecactide), elezanumab, elotuzumab (e.g. EMPLICITI), encorafenib, enfuvirtida (e.g. FUZEON), entinostat, entospletinib, enzastaurin, epacadostat, epirubicin, epratuzumab, eritoran tetrasodium, etanercept, etoposide, etrolizumab, everolimus, evobrutinib, filgotinib, fingolimod (e.g. fingolimod hydrochloride), firategrast, fludarabine, fluorouracil, fontolizumab, forodesine hydrochloride, fostamatinib, galunisertib, ganetespib, ganitumab, gemcitabine, gemtuzumab ozogamicin, gerilimzumab, glasdegib, glassia, glatiramer acetate, glembatumumab vedotin, glesatinib, golimumab (e.g. SIMPONI), guadecitabine, hydrocortisone, hydroxychloroquine sulfate, hydroxyurea, ibritumomab tiuxetan, ibrutinib, ibudilast, idarubicin, idebenone, idelalisib, ifosfamide, iguratimod, imatinib, imexon, IMU-838, infliximab, inotuzumab ozogamicin, interferon alfa-2, interferon beta-la, interferon beta-1b, interferon gamma-1, ipilimumab, irofulven, isatuximab, ispinesib, itacitinib, ixazomib, lapatinib, laquinimod, laromustine, ld-aminopterin, leflunomide, lenalidomide, lenvatinib, letrozole (e.g. FEMARA), levamisole, levocabastine, lipoic acid, lirilumab, lonafarnib, lumiliximab, maraviroc (e.g. SELZENTRY), masitinib, mavrilimumab, melphalan, mercaptopurine, methotrexate, methoxsalen, methylprednisone, milatuzumab, mitoxantrone, mizoribine, mocetinostat, monalizumab, mosunetuzumab, motesanib diphosphate, moxetumomab pasudotox, muromonab-CD3, mycophenolate mofetil (e.g. mycophenolate mofetil hydrochloride), mycophenolic acid, namilumab, natalizumab, navitoclax, neihulizumab, nerispirdine, neurovax, niraparib, nivolumab, obatoclax mesylate, obinutuzumab, oblimersen sodium, ocrelizumab, ofatumumab, olokizumab, opicinumab, oprelvekin, osimertinib, otelixizumab, oxaliplatin, oxcarbazepine, ozanimod, paclitaxel, pacritinib, palifermin, panobinostat, pazopanib, peficitinib, pegfilgrastim (e.g. NEULASTA), peginterferon beta-la, pegsunercept (peg stnf-ri), pembrolizumab, pemetrexed, penclomedine, pentostatin, perifosine, pevonedistat, pexidartinib, picoplatin, pidilizumab, pivanex, pixantrone, pleneva, plovamer acetate, polatuzumab vedotin, pomalidomide, ponatinib, ponesimod, prednisone/prednisolone, pyroxamide, R-411, ravulizimab-cwvz (e.g. (ULTOMIRIS), recombinant il-12, relatlimab, rhigf-1, rhigm22, rigosertib, rilonacept, ritonavir (e.g. NORVIR), rituximab, ruxolitinib, SAR442168/PRN2246, sarilumab, secukinumab, selumetinib, simvastatin, sintilimab, siplizumab, siponimod (e.g. MAYZENT), sirolimus (rapamycin), sirukumab, sitravatinib, sonidegib, sorafenib, sotrastaurin acetate, sunitinib, sunphenon epigallocatechin-gallate, tabalumab, tacrolimus (e.g. tacrolimus anhydrous), talabostat mesylate, talacotuzumab, tanespimycin, tegafur/gimeracil/oteracil, temozolomide, temsirolimus, tenalisib, terameprocol, teriflunomide, thalidomide, thiarabine, thiotepa, tipifarnib, tirabrutinib, tislelizumab, tivozanib, tocilizumab, tofacitinib, TR-14035, tregalizumab, tremelimumab, treosulfan, ublituximab, umbralisib, upadacitinib, urelumab, ustekinumab, varlilumab, vatelizumab, vedolizumab, veliparib, veltuzumab, venetoclax, vinblastine, vincristine, vinorelbine ditartrate, visilizumab, vismodegib, vistusertib, voriconazole (e.g. VFEND), vorinostat, vosaroxin, ziv-aflibercept or any combination thereof. In some embodiments, the immunosuppressive agent is 2B3-201, 3PRGD2, 4SC-202, 506U78, 6,8-bis(benzylthio) octanoic acid, 68Ga-BNOTA-PRGD2, 852A, 89Zr-DFO-CZP, ABBV-257, ABL001, ABP 501, ABP 710, ABP 798, ABT-122, ABT-199, ABT-263, ABT-348, ABT-494, ABT-555, ABT-874, ABX-1431 HCl, ACP-196, ACP-319, ACT-128800, ACY-1215, AD 452, Ad-P53, ADCT-301, ADCT-402, ADL5859, ADS-5102, AFX-2, AGEN1884, AGEN2034, AGS67E, AlN457, AK106-001616, ALD518, ALKS 8700, ALT-803, ALT-803, ALX-0061, ALXN1007, ALXN6000, AMD3100, AMG 108, AMG 319, AMG 357, AMG 570, AMG 592, AMG 714, AMG 719, AMG 827, AMP-110, AP1903, APL A12, APO866, APX005M, AQ4N, AR-42, ARN-6039, ARQ 531, ARRY-371797, ARRY-382, ARRY-438162, ART-102, ART621, ASK8007, ASN002, ASP015K, ASP1707, ASP2408, ASP2409, ASP5094, AT-101, AT7519M, AT9283, ATA188, ATN-103, ATX-MS-1467, AVL-292, AVP-923, AZD4573, AZD5672, AZD5991, AZD6244, AZD6738, AZD9056, AZD9150, AZD9567, AZD9668, B-701, BAF312, BAY1830839, BBI608, BCD-054, BCD-055, BCD-063, BCD-089, BCD-100, BCD-132, BCD-145, BEZ235, BG00012, BG9924, BGB-3111, BGB-A333, BGG492, BHT-3009, BI 655064, BI 695500, BI 695501, BI 836826, BI-1206, BIBR 796 BS, BIIB017, BIIB023, BIIB057, BIIB061, BIIL 284 BS, BLZ945, BMMNC, BMN 673, BMS-247550, BMS-582949, BMS-817399, BMS-936558, BMS-936564, BMS-945429, BMS-986104, BMS-986142, BMS-986156, BMS-986195, BMS-986205, BMS-986213, BMS-986226, BMS-986251, BNC105P, BOW015, BP1001, BT061, BTT-1023, C105, CAL-101, CAM-3001, CAT-8015, CB-839, CBL0137, CC-1088, CC-115, CC-122, CC-292, CC100, CCI-779, CCX 354-C, CDKI AT7519, CDP323, CDP6038, CDP870, CDX-1127, CDX-301, CE-224535, CF101, CFZ533, CGP 77116, CH-1504, CH-4051, CHR-5154, CHS-0214, CK-2017357, CLAG-M, CLR 131, CMAB008, CMP-001, CNF2024 (BIIB021), CNM-Au8, CNTO 1275, CNTO 136, CNTO 148, CNTO 6785, CP-195543, CP-461, CpG 7909, CPI-1205, CR6086, CRx-102, CS-0777, CS1002, CT-011, CT-1530, CT-P10, CV301, CX-3543, DAC-HYP, DCDT2980S, DI-B4, DPA-714 FDG, DS-3032b, DT2219ARL, DTRM-505, DTRM-555, DTRMWXHS-12, DWP422, E6011, E7449, EK-12, ELND002, ENIA11, EOC202, ETBX-011, F8IL10, FBTA05, FEDAA1106 (BAY85-8101), FGF401, FKB327, FPA008, FR104, FS118, FTY720, G100, GCS-100, GDC-0199, GDC-0853, GEH120714, GLPG0259, GLPG0634, GNbAC1, GNKG168, GP2013, GP2015, GRN163L, GS-1101, GS-5745, GS-9219, GS-9820, GS-9876, GS-9901, GSK1223249, GSK1827771, GSK2018682, GSK21110183, GSK239512, GSK2618960, GSK2831781, GSK2982772, GSK3117391, GSK3152314A, GSK3196165, GSK3358699, GSK706769, GW-1000-02, GW274150, GW406381, GW856553, GZ402668, HCD122, HE3286, HL2351, HL237, hLL1-DOX (IMMU-115), HLX01, HM71224, HMPL-523, HSC835, HZT-501, ICP-022, IDEC-C2B8, ILV-094, IMGN529, IMMU-114, IMO-2125, INCAGN02385, INCB018424, INCB028050, INCB039110, INCB047986, INCMGA00012, INNO-406, INT131, INT230-6, INVAC-1, IPI-145, IPX056, ISF35, ISIS 104838, ITF2357, JCARH125, JHL1101, JNJ 38518168, JNJ-39758979, JNJ-40346527, JNJ-63723283, JS001, JTE-051, JTX-2011, KB003, KD025, KPT-330, KW-2449, KW-2478, KX2-391, L-778123, LAG525, LAM-002A, LBEC0101, LBH589, LFB-R603, LMB-2, LX3305, LY2127399, LY2189102, LY2439821, LY3009104, LY3090106, LY3300054, LY3321367, LY3337641, M2951, M7824, M923, MBG453, MBP8298, MBS2320, MD1003, MDG013, MDV9300, MDX-1100, MDX-1342, MDX-1411, ME-401, MEDI-522, MEDI-538, MEDI-551, MEDI4920, MGA012, MGCD0103, MGD007, MIS416, MK-0873, MK-4280, MK-4827, MK-8457, MK-8808, MK0359, MK0457, MK0752, MK0782, MK0812, MK2206, MLN1202, MLTA3698A, MM-093, MN-122, MN-166, monoclonal antibody M-T412, monoclonal antibody mono-dgA-RFB4, MOR00208, MOR103, MORAb-022, MP-435, MP470, MRC375, MRG-106, MS-533, MSB11022, MSC2490484A, MT-1303, MT-3724, MTIG7192A, MTRX1011A, NBI-5788, NC-503, NI-0101, NI-071, NIS793, NKTR-214, NNC 0141-0000-0100, NNC 0151-0000-0000, NNC0109-0012, NNC0114-0000-0005, NNC0114-0006, NNC0142-0002, NNC0215-0384, NNC109-0012, NOX-A12, NT-KO-003, NU100, OMB157, OMP-313M32, ON01910 Na, ONO-2506PO, ONO-4641, ONTAK, OPB 31121, OSI-461, OTS167IV, P1446A-05, PBF-509, PBR06, PCI 32765, PCI-24781, PD 0360324, PDA001, PDR001, PF-04171327, PF-04236921, PF-04308515, PF-04629991, PF-05280586, PF-06342674, PF-06410293, PF-06438179, PF-06650833, PF-06651600, PF-06835375, PG-760564, PH-797804, PLA-695, PLX3397, PLX5622, POL6326, PRO131921, PRO283698, PRTX-100, PS-341, PTL201, R(−) XK469, R788, RAD001, RC18, REGN1979, REGN3767, REGN2810, REGN4659, RFT5-SMPT-dgA, RG2077, RGB-03, RGI-2001, RHB-104, RNS60, RO5045337, RO7123520, Rob 803, RPC1063, RWJ-445380, S 55746, SAIT101, SAN-300, SAR245409, SB-681323, SB683699, SBI-087, SC12267 (4SC-101), SCH 727965, SCIO-469, SD-101, SG2000, SGN-40, SHC014748M, SHR-1210, SHR0302, SHR1020, SJG-136, SKI-O-703, SMP-114, SNS-032, SNS-062, SNX-5422, SPARC1103 I, SPC2996, SSR150106, STA 5326 mesylate, Sunpharma1505, SyB L-0501, Sym022, Sym023, SYN060, T-614, T0001, TA-650, TAB08, TAK-715, TAK-783, TAK-901, TGR-1202, TH-302, TL011, TMI-005, TMP001, TNFa Kinoid, TP-0903, TRU-015, TRU-016, TSR-022, TSR-033, TSR-042, TXA127, VAY736, VP-16, VSN16R, VX-509, VX-702, VX-745, VX15/2503, XCEL-MC-ALPHA, XL228, XL844, XmAb13676, XmAb5574, XOMA 052, YRA-1909, Z102, ZEN003365, or any combination thereof. In some embodiments, the immunosuppressive agent is A2aR antagonist, Akt inhibitor, anti CD20, Anti-amyloidotic (AA) Agent, anti-CD37 protein therapeutic, anti-CTLA4 mAb, Anti-CXCR4, anti-huCD40 mAb, anti-LAG3 mAb, anti-PD-1 mAb, anti-PD-L1 agent, anti-PD-L1 agent, anti-PD-L1 mAb, anti-TGFb mAb, anti-TIGIT mAb, anti-TIM-3 mAb, Aurora kinase inhibitor, Bcl-2 Inhibitor, bifunctional fusion protein targeting TGFb and PD-L1, bispecific anti-PD-1 and anti-LAG3 mAb, CD1d ligand, CD40 agonist, Complement C5a inhibitor, CSF1R inhibitor, EZH2 inhibitor, FGFR3 inhibitor, FGFR4 inhibitor, FGFrR3 inhibitor, glucocorticoid-induced tumor necrosis factor receptor-related gene [GITR] agonist, glutaminase inhibitor, Human monoclonal antibody against IL-12, ICOS agonist, IDO1 inhibitor, IL2 mutein, IL2 receptor agonist, MEK inhibitor, multitargeted receptor tyrosine kinase inhibitor, neutrophil elastase inhibitor, Notch Inhibitor, p38 MAPK inhibitor, PD-1 inhibitor, recombinant human Flt3L, ROCK inhibitor, selective sphingosine-1-phosphate receptor modulator, Src kinase inhibitor, TLR4 agonist, TLR9 agonist, or any combination thereof. In some embodiments, the immunosuppressive agent is a Complement C5a inhibitor, a CD40 agonist, a p38 inhibitor, a CSF1R inhibitor, a MEK inhibitor, a neutrophil elastase inhibitor, FGFrR3 inhibitor, anti-LAG3 mAb, Anti-CXCR, glucocorticoid-induced tumor necrosis factor receptor-related gene [GITR] agonist, IDO1 inhibitor, ICOS agonist, glutaminase inhibitor, recombinant human Flt3L, TLR9 agonist, EZH2 inhibitor, anti-CTLA4 mAb, PD-1 inhibitor, PD-L1 inhibitor, anti-PD-L1 mAb, FGFR4 inhibitor, bispecific anti-PD-1 and anti-LAG3 mAb, TLR4 agonist, Bcl-2 Inhibitor, anti-LAG3 mAb, an inhibitor of a cell degradation pathways, or a proteasome inhibitor.

In some embodiments, the immunosuppressive agent is a sphingosine-1-phosphate receptor and/or nicotinic acetylcholine receptor modulator. In some cases, the immunosuppressive medications can be therapeutic antibodies, including Immunoglobulin G. In some cases, the immunosuppressive medications can be asparaginase inhibitors. In some cases, the immunosuppressive medications can be B-lymphocyte stimulator (BLyS)-specific inhibitor. In some cases, the immunosuppressive medications can be T-cell costimulation modulators. In some cases, the immunosuppressive medications can be cyclic polypeptide immunosuppressants and/or synthetic polypeptides that modify immune processes. In some cases, the immunosuppressive medications can be corticosteroids. In some cases, the immunosuppressive medications can be cytotoxic chemotherapy drugs. In some cases, the immunosuppressive medications can be cytotoxic glycopeptide antibiotics and/or mixtures thereof. In some cases, the immunosuppressive medications can be molecules that inhibit pro-inflammatory cytokine production. In some cases, the immunosuppressive medications can be thalidomide analogues. In some cases, the immunosuppressive medications can be inhibitors of RANKL (receptor activator of nuclear factor kappa-B ligand). In some cases, the immunosuppressive medications can be inhibitors of histone deacetylase (HDAC). In some cases, the immunosuppressive medications can be inhibitors of heat shock protein 90 (HSP90). In some cases, the immunosuppressive medications can be inhibitors of cytidine deaminase (CDA). In some cases, the immunosuppressive medications can be inhibitors of Hedgehog signaling pathway (including Sonic hedgehog and Smoothened). In some cases, the immunosuppressive medications can be inhibitors of alpha-1-proteinase. In some cases, the immunosuppressive medications can be inhibitors of cyclooxygenase 2 (COX2). In some cases, the immunosuppressive medications can be inhibitors of complement (C5a). In some cases, the immunosuppressive medications can be inhibitors of colony stimulating factor 1 receptor (CSF1R). In some cases, the immunosuppressive medications can be inhibitors of Notch. In some cases, the immunosuppressive medications can be inhibitors of kinesin. In some cases, the immunosuppressive medications can be inhibitors of farnesyltransferase. In some cases, the immunosuppressive medications can be inhibitors of poly(ADP-ribose) polymerase (PARP). In some cases, the immunosuppressive medications can be inhibitors of Neural Precursor Cell Expressed, Developmentally Down-Regulated (NEDD8). In some cases, the immunosuppressive medications can be inhibitors of dipeptidyl peptidase IV (DPP-IV). In some cases, the immunosuppressive medications can be inhibitors of leucine-rich repeat kinase 2 (LRRK2). In some cases, the immunosuppressive medications can be inhibitors of immune checkpoint proteins. In some cases, the immunosuppressive medications can be inhibitors of indoleamine 2,3-dioxygenase-1 (IDO1). In some cases, the immunosuppressive medications can be inhibitors of chemokine receptors (CCR4, CCR5, CCR7). In some cases, the immunosuppressive medications can be immunosuppression-inducing therapies such as T-cells or regulatory T-cells modified with a chimeric antigen receptor (CAR-T, CAR-Tregs). In some cases, the immunosuppressive medications can be structured lipids. In some cases, the immunosuppressive medications can be Ras mimetic. In some cases, the immunosuppressive medications can be inhibitors of NOD-like receptor pyrin domain-containing protein 3 (NLRP3). In some cases, the immunosuppressive medications can be mTOR and/or calcineurin inhibitors. In some cases, the immunosuppressive medications can be complement inhibitors. In some cases, the immunosuppressive medications can be immunosuppressive antimetabolites, nucleoside metabolic inhibitors, imidazole nucleosides, nucleotide analogs, nucleoside synthesis inhibitors, purine synthesis inhibitors, pyrimidine synthesis inhibitors, or pyrimidine synthase inhibitors. In some cases, the immunosuppressive medications can be recombinant proteins, such as recombinant interferon beta, IL-2, IL-11, Lymphotoxin B fusion protein, Therapeutic T cell receptor peptide vaccine, Keratinocyte growth factor, or Tumor necrosis factor (TNF) receptor.

In some cases, the immunosuppressive medications can be DNA and/or RNA crosslinking agents, including alkylating agents, nitrogen mustard alkylating agents, topoisomerase inhibitors, anthracyclines, and platinum-based anticancer drugs. In some cases, the immunosuppressive medications can be kinase inhibitors, including phosphoinositide-3-kinase, cyclin-dependent kinase (e.g., CDK9), Aurora kinase, ROCK, Akt, or PKC. In some cases, the immunosuppressive medications can be tyrosine kinase inhibitors, including inhibitors of the fusion protein breakpoint cluster region-Abelson murine leukemia viral oncogene homolog 1 (BCR-ABL), Bruton's tyrosine kinase (BTK), epidermal growth factor receptor (EGFR), Janus kinase (JAK), Syk, Lyn, MEK, FAK, BRAF, AXL, or vascular endothelial growth factor (VEGF). In some cases, the immunosuppressive medications can be monoclonal antibodies and/or antibody-drug conjugates directed at proteins including cluster of differentiation (CD) proteins, such as CD2, CD3, CD11a, CD20, CD30, CD52, CD-19, CD-38, CD-26, CD-37, CD-22, CD-33, CD-23, CD-74, CD-162, CD-79, CD-123, CD-4, CD-137, CD-27, CD-36, CD-39, CD-73, CD-226, CD-155, CD-40; interleukins (IL), such as IL-1, IL-2, IL-6, IL-12, IL-23; tumor necrosis factor (TNF) family proteins, such as TNFα; and integrins, such as integrin α4, αvβ3, αvβ5, αvβ3, or α2. In some cases, the immunosuppressive medications can be monoclonal antibodies and/or antibody-drug conjugates directed at Programmed cell death receptor 1 (PD-1), Programmed cell death ligand 1 (PD-L1), Cytotoxic T-lymphocyte associated protein 4 (CTLA-4), Lymphocyte activation gene 3 (LAG-3), T-cell immunoglobulin and mucin-domain containing-3 (TIM-3), T-cell immunoreceptor with Ig and ITIM domains (TIGIT), also known as WUCAM or Vstm3, B and T lymphocyte attenuator (BTLA), Glucocorticoid-induced TNFR family related gene (GITR), OX40, HSP90, killer-cell immunoglobulin-like receptor (KIR), Toll-like receptor 9 (TLR9), Toll-like receptor 4 (TLR4), Matrix metallopeptidase 9 (MMP), Interferon receptor, Interferon gamma, Transforming growth factor 1b (TGF1BD, Insulin growth factor 1 receptor (IGF1 R), Fibroblast growh factor receptor (FGFrR3, FGFR4), Neuromedin B, Granulocyte-macrophage colony stimulating factor receptor (GM-CSF R), Natural killer cell receptor (NKG-2a), Leucine rich repeat and Immunoglobin-like domain-containing protein 1 (LINGO1), B-cell activating factor (BAFF), Inducible T-cell co-stimulator (ICOS). In some cases, the monoclonal antibody/antibody-drug conjugate can activate the target.

In some embodiments, the subject with a B cell immunodeficiency can be currently treated with an immunosuppressive medication. In some embodiments, a subject can be previously treated with an immunosuppressive medication. In some embodiments, a subject can be not yet treated with an immunosuppressive medication. The immunosuppressive medication can include but not limited to glucocorticoids, cytostatics, antibodies, drugs acting on immunophilins, interferons, opioids, TNF binding proteins, mycophenolate, or other small biological agents. For example, glucocorticoids can include but not limited to cortisol (hydrocortisone), cortisone, prednisone, prednisolone, methylprednisolone, dexamethasone, betamethasone, triamcinolone, beclometasone, fludrocortisone acetate, deoxycorticosterone acetate (DOCA), or aldosterone. Cytostatics can include but not limited to nitrogen mustards (e.g., cyclophosphamide), nitrosoureas, platinum compounds, folic acid analogues such as methotrexate, purine analogues such as azathioprine and mercaptopurine, pyrimidine analogues such as fluorouracil, protein synthesis inhibitors, cytotoxic antibiotics such as dactinomycin, anthracyclines, mitomycin C, bleomycin, or mithramycin. Antibodies can include but not limited to polyclonal antibodies such as atgam and thymoglobuline, monoclonal antibodies such as CD25- and CD3-directed antibodies, muromonab-CD3, basiliximab (e.g., SIMULECT), and daclizumab (e.g., ZENAPAX). Drugs acting on immunophilins can include but not limited to ciclosporin, tacrolimus, sirolimus, or everolimus. TNF binding proteins can include but not limited to infliximab (e.g., REMICADE), etanercept (e.g., ENBREL), or adalimumab (e.g., HUMIRA). Other small biological agents can include but not limited to fingolimod, myriocin, and rituximab (e.g., RITUXAN).

In some embodiments, the subject with a B cell immunodeficiency can be diagnosed or undiagnosed with a condition (e.g., disease or disorder), can be asymptomatic or symptomatic, can have increased or decreased susceptibility to a condition (e.g., disease or disorder), can be currently under or previously under or not under a treatment for a condition (e.g., disease or disorder), or any combination thereof. In some embodiments, the condition can be AIDS, cancer, organ transplant, or an autoimmune disease. In some embodiments, the condition can be an age-related decline in immunity, or immunosenescence, or multifactoral immunodeficiency, or an age-related immuodeficiency.

In some embodiments, the subject with a B cell immunodeficiency can be diagnosed or undiagnosed with AIDS (e.g., individuals infected with HIV), can be asymptomatic or symptomatic, can have increased or decreased susceptibility to AIDS, can be currently under or previously under or not under a treatment for AIDS, or any combination thereof. In some embodiments, a subject can be diagnosed or undiagnosed with cancer (e.g., Hodgkin's disease, leukemia, lymphoma, or myelofibrosis), can be asymptomatic or symptomatic, can have increased or decreased susceptibility to cancer, can be currently under or previously under or not under a treatment for cancer, or any combination thereof. In some embodiments, a subject can be currently diagnosed or previously diagnosed or undiagnosed with an autoimmune disease (e.g., multiple sclerosis, rheumatoid arthritis, psoriasis, systemic lupus erythematosus), can be asymptomatic or symptomatic, can have increased or decreased susceptibility to an autoimmune disease, can be currently under or previously under or not under a treatment for an autoimmune disease, or any combination thereof.

In some embodiments, the string vaccine described herein may confer resistance, cross protection and generate immunogenicity against other SARS viruses or to a variety of viral strains having similarity to the 2019 SARS-Cov 2.

### Kits

The viral epitope therapeutic described herein can be provided in kit form together with instructions for administration. Typically, the kit would include the desired antigen therapeutic in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

The present disclosure will be described in greater detail by way of specific examples. The following examples are offered for illustrative purposes, and are not intended to limit the present disclosure in any manner. Those of skill in the art will readily recognize a variety of non-critical parameters that can be changed or modified to yield alternative embodiments according to the present disclosure. All patents, patent applications, and printed publications listed herein are incorporated herein by reference in their entirety.

### EXAMPLES

### Example 1: Sequence-Based Prediction of Vaccine Targets for Inducing T Cell Responses to SARS-COV-2

Coronaviruses are positive-sense single-stranded RNA viruses that have occasionally emerged from zoonotic sources to infect human populations. Most coronavirus infections cause mild respiratory symptoms. However, some recent coronavirus infections have resulted in serious morbidity and mortality, including the severe acute respiratory syndrome coronavirus (SARS-CoV), Middle East respiratory syndrome coronavirus (MERS-CoV) and 2019 SARS-CoV-2, which is responsible for the current worldwide pandemic, COVID-19. These three viruses belong to the genus Betacoronaviridae. SARS-CoV was identified in South China in 2002 and its global spread led to 8,096 cases and 774 deaths. The first case of MERS-CoV emerged in 2012 in Saudi Arabia, and since then a total of 2,494 cases and 858 associated deaths have been reported. In contrast to the more limited scope of these other coronavirus infections, SARS-CoV-2, which emerged in Wuhan, China at the end of December 2019, has resulted in 4,077,355 cases, including 279,043 deaths globally as of May 9, 2020. The rapid spread of SARS CoV-2 has resulted in the World Health Organization declaring a global pandemic. Thus, there is an urgent need for effective vaccines and antiviral treatments against SARS CoV-2 to reduce the spread of this highly infectious agent.

The genome of SARS CoV-2 spans 30 kilobases in length and encodes for 13 open reading frames (ORFs), including four structural proteins. These structural proteins are the spike protein(S), the membrane protein (M), the envelope protein (E), and the nucleocapsid protein (N). In addition, there are over 20 non-structural proteins that account for all the proteins involved in the transcription and replication of the virus. All encoded proteins of the virus are potential candidates for developing vaccines to induce robust T cell immunity.

SARS-CoV and SARS CoV-2 share 76% amino acid identity across the genome. This high degree of sequence similarity allows us to leverage the previous research on protective immune responses to SARS-CoV to aid in vaccine development for SARS-CoV-2. Both humoral and cellular immune responses have been shown to be important in host responses to SARS-CoV. Antibody responses generated against the S and the N proteins have shown to protect from SARS-CoV infection in mice and have been detected in SARS-CoV infected patients. However, the antibody responses detected against the S protein were undetectable in patients six years post-recovery. In addition, higher titers of antibodies have been found in more severe clinical cases of viral infection suggesting that a robust antibody response alone may be insufficient for controlling SARs-COV and SARS CoV-2 infection.

Together with B cell immunity, T cell responses seem important in the immune response's control of SARS-CoV and is most likely important for the control of SARS-CoV-2. In mice, studies have shown that adoptive transfer of SARS-CoV-specific memory CD8+ T cells provided protection against a lethal SARS-CoV infection in aged mice and that adoptive transfer of effector CD4+ and CD8+ T cells to immunodeficient or young mice expedited virus clearance and improved clinical results. Both CD4+ and CD8+ T cell responses have also been detected in SARS-CoV and SARS-CoV-2-infected patients. Additionally, SARS-CoV specific memory CD8+ T cells have been found to persist for up to 11 years post-infection in patients who recovered from SARS. These viral specific CD8+ T cells can be cytotoxic and can kill virally infected cells to reduce disease severity. In addition to having effector functions, CD4+T cells can promote the production of virus-specific antibodies by activating T-dependent B cells. Given the wealth of data from SARS-CoV, the homology between the SARS CoV-2 and SARS-CoV, as well as emerging data from SARS-CoV-2, T cell immunity likely plays a critical role in providing protection against SARS-CoV-2.

Here, we utilized mass spectrometry (MS)-based HLA-I and HLA-II epitope binding prediction tools to identify SARS CoV-2 epitopes recognized by CD4+ and CD8+ T cells. These binding predictors were trained on high-quality mono-allelic HLA immunopeptidome data generated via MS. The use of MS for the identification of MHC peptide ligandome yields an extensive and relatively unbiased population of naturally processed and presented MHC binding peptides in vivo. Unlike traditional binding assays which rely on chemical synthesis and a priori knowledge of peptides and ligands to be assayed, MS uses natural peptide-MHC complexes which are subject to the endogenous processing and presentation pathways within the cell. Additionally, the use of engineered mono-allelic cell lines avoids dependence on in-silico deconvolution techniques and allows for allele coverage to be expanded in a targeted manner.

With this approach, we generated binding predictors for 74 HLA-I and 83 HLA-II alleles. Alleles selected for data collection were prioritized to maximize population coverage. This MS data enabled us to train allele-specific neural network-based binding predictors that outperform the leading affinity-based predictors for both HLA-I and HLA-II. Furthermore, we demonstrated in Abelin et al., 2019 that this improved binding prediction led to improved immunogenicity prediction by validating this approach on a data set of immune responses to a diverse collection of pathogens and allergens. Here, we specifically validated the binding predictors utilizing Coronaviridae family peptides that had been assayed for T cell reactivity or MHC binding from the Virus Pathogen Resource (ViPR) database. The ViPR database integrates viral pathogen data from internally curated data, researcher submissions, and data from various external sources. Our approach provides a significant improvement in both the breadth of predictions, and their validity, compared with a recent study that had a similar aim, but relied upon a smaller validation data set and fewer covered alleles, leading to a much more limited set of bioinformatically predicted SARS CoV-2 epitopes.

We used our MS-based HLA-I and HLA-II binding predictors to predict the binding potential of peptide sequences from across the entire SARS CoV-2 genome for a broad set of HLA-I and HLA-II alleles, covering the vast majority of USA, European, and Asian populations. We additionally confirm that a subset of these epitopes can raise specific CD8+ T cell responses in T cell induction assays using donor PBMCs. Furthermore, we interrogate publicly available proteomics data and demonstrate that the relative expression of multiple SARS CoV-2 proteins in virally infected cells vary significantly, indicating another parameter that should be considered in vaccine design to induce cellular immunity. Epitopes predicted to have a high likelihood of binding to multiple HLA-I and HLA-II alleles and exhibit high expression in infected human cells are promising vaccine candidates to elicit T cell responses against SARS-CoV-2.

### METHODS

Analysis of Coronaviridae Family T Cell Epitopes from ViPR

Experimentally determined epitopes for the Coronaviridae family for human hosts were retrieved from the ViPR database (viprbrc.org/; accessed Mar. 5 2020). To build a validation dataset, both positives and negatives for T cell assays and MHC binding assays were obtained. Only assays associated with alleles identified with at least four-digit resolution and supported by our predictors were included for this analysis.

Positive calls were prioritized-that is, if a given peptide-allele pair was assayed multiple times by a specific assay type and was determined to be positive in any single one of the assays, the peptide-allele pair was classified as positive. Specifically, the priority was given by the following order: Positive-High>Positive-Intermediate>Positive-Low>Positive>Negative (e.g., a peptide allele pairing that was assayed three times with the results Positive-High, Positive, and Negative were assigned a Positive-High result). Of note, alternative approaches such as prioritizing negative assay results, or random choice in cases of multiple results, yielded very similar results (data not shown).

### Binding Prediction for ViPR Coronaviridae Family T Cell Epitopes

Peptide-HLA-I allele pairs in the ViPR validation dataset were scored using our HLA-I binding predictor, a neural network trained on mono-allelic MS data. Similarly, peptide-HLA-II allele pairs in the ViPR validation dataset were scored using our HLA-II binding predictor, a recently published convolutional neural network-based model also trained on mono-allelic MS data. We scored all 12-20mers contained within a given assay peptide with the HLA-II binding predictor and took the maximum score as the representative binding score for the assay peptide. In vitro MHC binding assays, which represent the vast majority of the ViPR dataset, do not require endogenous processing and presentation for a positive binding result. Since our binding predictor, which is trained on naturally processed and presented ligands observed via MS, is also implicitly learning these endogenous processing rules, we score all potential ligands within an assayed peptide (rather than just the full-length assay peptide itself) to account for this distinction.

### Retrieval of 2019 SARS CoV-2 Sequence

The GenBank reference sequence for 2019 SARS CoV-2 (accession: NC_045512.2) was used for this study. All twelve annotated open reading frames (ORF1a, ORF1b, S, ORF3a, E, M, ORF6, ORF7a, ORF7b, ORF8, N, and ORF10) were considered as sources of potential epitopes. In addition, due to its high expression level in recently published proteomic datasets, ORF9b, as annotated by UniProt (PODTD2), was also used for epitope predictions.

### Identification of HLA-I Epitopes and Prioritization by Population Coverage

To identify candidate HLA-I epitopes, we exhaustively scored all possible 8-12mer peptide sequences from 2019 SARS CoV-2 with our HLA-I binding predictor for 74 alleles, including 21 HLA-A alleles, 35 HLA-B alleles, and 18 HLA-C alleles. Peptide-allele pairs were assigned a percent rank by comparing their binding scores to those of 1,000,000 reference peptides (selected from a partition of the human proteome that had not been used for model training) for the same respective allele. Peptide-allele pairs that scored in the top 1% of the scores of these reference peptides were considered strong potential binders.

Since a vaccine should ideally benefit a large fraction of the population, these top-ranking peptides were then prioritized based on expected population coverage, given all the alleles each peptide was expected to bind to (i.e., all the alleles for which the peptide scored in the top 1%). The estimate of population coverage for each peptide was calculated as:

coverage=1-Π_{loci}(1-Σ_{locus alleles} f_{allele,avg)}²

where f_{allele,avg} is the (unweighted) average allele frequency across the USA, European, and Asian Pacific Islander (API) populations, and the cumulative product taken across the three HLA-I loci: HLA-A, HLA-B, and HLA-C. The cumulative product itself represents the chance that an individual in the population does not express any one of the contained alleles; hence, the complement describes the probability that at least one is present.

The USA population allele frequency is calculated as the following weighted average of a few subpopulations: 0.623*EUR+0.133*AFA+0.068*API+0.176*HIS, where EUR=European, AFA=African American, API=Asian Pacific Islander, and HIS=Hispanic populations. For alleles where AFA, HIS, or API population frequencies were not available, the USA population allele frequency values were set to match EUR. Missing API allele frequency values were conservatively imputed with 0 for our analyses.

We then constructed two types of ranked lists of HLA-I epitopes by coverage. The first ranks all SARS CoV-2 epitopes by their absolute coverage, such that peptides predicted to bind similar collections of alleles would be ranked similarly This approach provides the full list of predicted class I epitopes sorted by the expected coverage for each peptide, with the generous assumption that every binding prediction is correct.

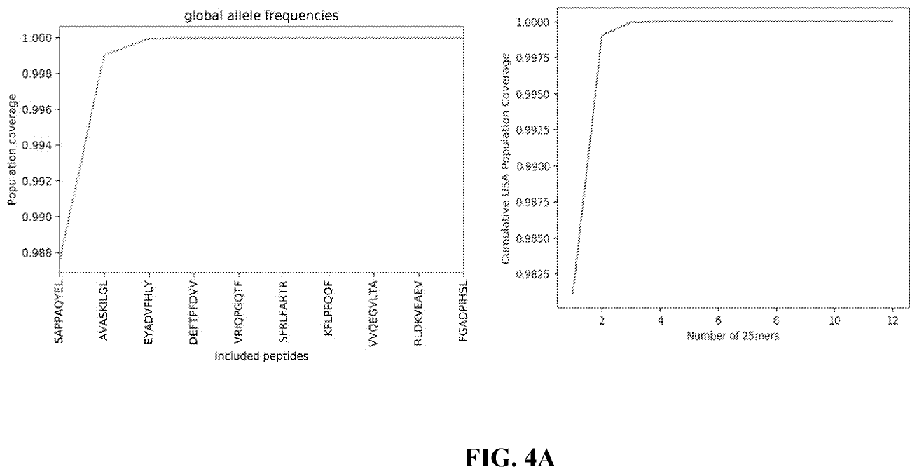

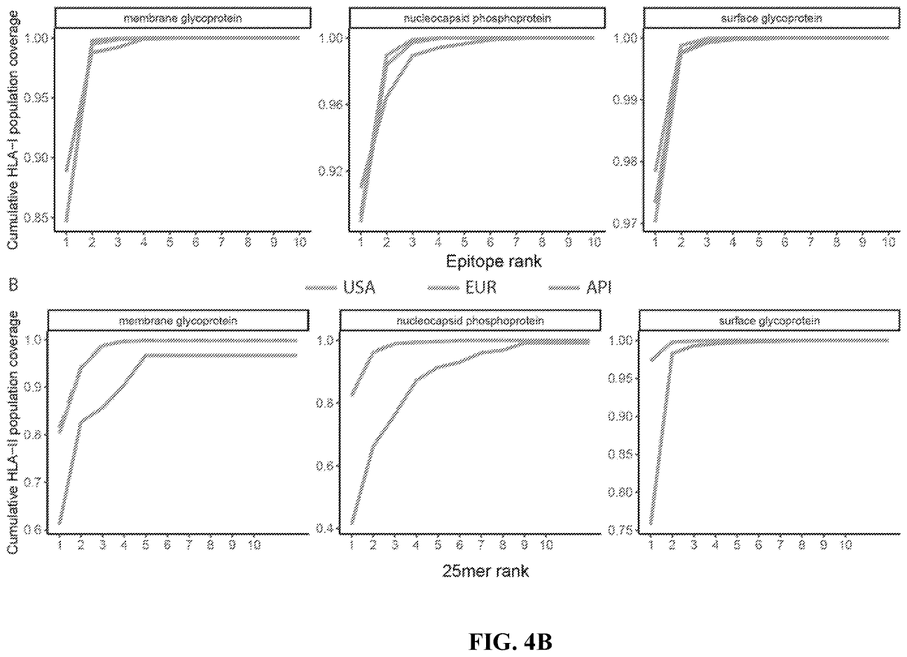

The second type of list, referred to as a “disjoint” list, is constructed in an iterative fashion where the peptide with the greatest coverage is selected first, and then the coverage for the remaining epitopes is updated to nullify contributions from any alleles that have already been selected (Table 6). Disjoint lists were generated for M, N, and S proteins (the most highly expressed structural proteins) individually, instead of across the entire 2019 SARS CoV-2 genome, to provide protein-level prioritizations. This approach produces a parsimonious list of peptides that is designed to maximize cumulative population coverage with the fewest number of selections and was used to generate FIG. 4A (left), and FIG. 4B (Upper Panel).

### Identification of HLA-II Epitopes and Prioritization by Population Coverage

To identify HLA-II epitopes, we used our MS-based HLA-II binding predictor to score all 12-20mer sequences in the SARS CoV-2 proteome to predict both binding potential and the likely binding core within each 12-20mer. Scoring was performed across all supported HLA-II alleles, consisting of 46 HLA-DR alleles, 17 HLA-DP alleles, and 20 HLA-DQ alleles.

Peptide-allele pairs were assigned a percent rank by comparing their binding scores to those of 100,000 reference peptides (as before, sampled from a partition of the human proteome that was held out from training). Pairs scoring in the top 1% were deemed likely to bind. Additionally, we define the “epitope” of a 12-20mer to be the predicted binding core within the sequence. As such, overlapping 12-20mers with the same predicted binding core for a given allele would constitute a single epitope. Table 5 shows counts of these epitopes.

Additionally, we prioritized predicted HLA-II binding 25mers in SARS CoV-2 by population coverage, given the desire to design vaccines that are effective broadly across the global population. To do this, we associated each 25mer with all subsequences that were likely binders and calculated the population coverage of the corresponding HLA-II alleles. Given a collection of alleles, we calculated the coverage as described in the previous section, the only difference being the cumulative product is taken across the following four HLA-II loci: HLA-DRB1, HLA-DRB3/4/5, HLA-DP, and HLA-DQ. HLA-II allele frequencies were obtained from and Allele Frequency Net Database.

As with HLA-I, two types of sorted lists of predicted binding sequences were generated. The first type ranks every predicted SARS CoV-2 25mer by absolute coverage provided by the HLA-II alleles to which a constituent subsequence is expected to bind. The second type of ranking was again performed for predicted binders in M, N, and S proteins individually, using disjoint coverage, to maximize cumulative population coverage with a parsimonious list of peptides (Table 7). This was used to generate FIG. 4A (right), and FIG. 4B (Lower Panel).

### Comparison of Predicted Epitopes to the Human Proteome

8-12mer sequences (corresponding to predicted HLA-I epitopes), 9mer sequences (corresponding to predicted HLA-II binding cores), and 25mer sequences (corresponding to predicted HLA-II sequences that bound multiple alleles) from SARS CoV-2 were compared against all subsequences of the same length from the human proteome, using UCSC Genome Browser genes with hg19 annotation of the human genome and its protein coding transcripts (63,691 entries). Exact matches were identified and omitted from the disjoint coverage ranking analysis to avoid prioritizing peptides that may inadvertently induce an autoimmune response. No exact matches were found for the predicted HLA-II binding cores or 25mer sequences.

### T Cell Induction and Assessment of Peptide-MHC Positive T Cell Responses

Human PBMCs from HLA-A02:01-positive human donors were isolated using Ficoll separation from apheresis material (AllCells, USA). Twenty three SARS CoV-2 epitopes predicted to be strong binders to HLA-A02:01 were pooled by similar binding potential, with up to 6 peptides per pool. The selected peptides represent high ranking peptides predicted to bind HLA-A02:01 from across the S, N, M, E, and ORF1ab proteins, avoiding sequences also prioritized by Grifoni et al. Five of the 23 peptide sequences are also found in SARS-CoV and were previously assayed and confirmed as HLA-A02:01 binders in ViPR. PBMCs were incubated with peptide pools, matured, and cultured in the presence of IL-7 and IL-15 (CellGenix GmbH, Germany) to promote T cell growth. Cells were then harvested and the frequency of CD8+ T cells specific to peptide-MHC (pMHC) were assayed using combinatorial coding of pMHC multimers. pMHC multimers were prepared as described elsewhere by the Applicants. Briefly, biotinylated HLA-A02:01 monomers loaded with UV cleavable peptides were exchanged under UV light with SARS CoV-2 predicted peptides. The streptavidin labelled fluorophores PE, APC, BV421 (Biolegend, Inc., USA), BV650 and BUV395 (BD Biosciences, USA) were added to UV exchanged monomers to create fluorescently labelled multimer reagents. Harvested cells were then stained with LIVE/DEAD Fixable Near-IR Dead Cell Stain Kit for 633 or 635 nm excitation (Life Technologies Corporation, USA), anti-CD4 FITC, anti-CD14 FITC, anti-CD16 FITC, and anti-CD19 FITC (BD Biosciences, USA) and anti-CD8 AF700 (Biolegend Inc., USA). Only live CD8+ T cells staining for both fluorochromes of the relevant pMHC multimers were considered positive. Samples were analyzed on FACS LSR Fortessa 18 X20 cytometers (BD Biosciences) and data was analyzed using FlowJo (TreeStar). The respective peptides used in the assay are displayed in Table 4.

TABLE 4
Viral epitopes and T cell induction

| | | No. Donors with | |
|---|---|---|---|
| | | pMHC specific | Confirmed |
| Protein | Sequence | T tells | in VIPR |
| Envelope protein (E) | FLAFVVFLL | | |
| Envelope protein (E) | FLAFVVFLLV | | |
| Envelope protein (E) | FLLVTLAIL | | |
| Envelope protein (E) | FLAFVVFL | 2 | |
| Membrane glycoprotein (M) | FLWLLWPVTL | | |
| Membrane glycoprotein (M) | FLYIIKLIFL | | |
| Membrane glycoprotein (M) | FLFLTWICL | 2 | |
| Membrane glycoprotein (M) | KLLEQWNL | | |
| Nucleocapsid phosphoprotein (N) | KLDDKDPNF | 2 | |
| Nucleocapsid phosphoprotein (N) | DLDDFSKQL | | |
| Nucleocapsid phosphoprotein (N) | YLGTGPEAGL | 2 | |
| Nucleocapsid phosphoprotein (N) | ILLNKHIDA | | + |
| Nucleocapsid phosphoprotein (N) | LLLDRLNQL | 1 | + |
| ORF1ab polyprotein | FGDDTVIEV | 3 | |
| ORF1ab polyprotein | FLLPSLATV | 2 | |
| ORF1ab polyprotein | WLMWLIINL | | |
| ORF1ab polyprotein | ALWEIQQV | | |
| Spike protein (S) | SLIDLQEL | | |
| Spike protein (S) | NLNESLIDL | | + |
| Spike protein (S) | KLNDLCFTNV | 2 | |
| Spike protein (S) | GLTVLPPLL | 1 | |
| Spike protein (S) | FIAGLIAIV | 2 | + |
| Spike protein (S) | RLQSLQTYV | 3 | + |

### Analysis of Publicly Available SARS CoV-2 Proteomic Datasets

2019 SARS CoV-2 proteomic datasets were downloaded from the PRIDE repository (Bojkova et al.: PXD017710; Bezstarosti et al.: PXD018760; Davidson et al.: PXD018241). In these studies, either Caco-2 human colorectal adenocarcinoma cells (Bojkova) or Vero E6 African green monkey kidney epithelial cells (Bezstarosti and Davidson) were subject to infection with 2019 SARS-CoV-2. Tandem mass spectra (MS/MS) acquired with data-dependent acquisition (DDA) were interpreted using Spectrum Mill MS Proteomics software package v7.0 pre-release (Agilent Technologies). Cysteine carbamidomethylation was selected as a fixed modification. Methionine oxidation, asparagine deamidation, protein N-termini acetylation, peptide N-terminal glutamine to pyroglutamic acid, and peptide N-terminal cysteine pyro-carbamidomethylation were selected as variable modifications. For the Bojkova dataset which employed isobaric mass tags, TMT11 was added as a fixed modification to peptide N-termini and lysines, and 13C6-15N2-TMT11-lysine and ¹³C₆-¹⁵N₄-arginine were added as variable modifications. All datasets were searched against the 2019 SARS CoV-2 proteome (UniProtKB, 28 Apr. 2020, 14 entries) concatenated to databases containing either the Homo sapiens proteome (Bojkova, UCSC Genome Browser hg19 annotation, 63691 entries) or the Chlorocebus sabaeus proteome (Bezstarosti and Davidson, UniProtKB, 9229 entries). Precursor and fragment mass tolerances were set as described in each manuscript, or as 20 ppm when not specified. Database search results were exported as a list of peptide-spectrum matches (PSMs) with a target-decoy based false discovery rate (FDR) estimation of 1%. Individual fractions from each study were combined into a single list. To perform spectral counting, PSMs assigned to a single 2019 SARS CoV-2 protein were counted, with ORF1a and ORF1ab treated as a single protein group. Peptides matched to both a host and SARS CoV-2 protein were discarded. Spectral counts were normalized to the length of each protein, and the maximum value within each dataset was set to 100%.

### RESULTS

### Bioinformatics Predictor Validation for Viral Epitopes Using ViPR

We first sought to validate the ability of our predictors to identify epitopes from genomes of the Coronaviridae family. Since SARS CoV-2 only emerged recently, specific data on SARS CoV-2 peptide MHC-binding and immunogenic epitopes are currently limited. However, other viruses from the Coronaviridae family have been studied thoroughly, specifically MERS-CoV and SARS-CoV. The latter has significant sequence homology to 2019 SARS-CoV-2. We therefore sought to leverage previously tested epitopes from across the Coronaviridae family to validate our predictions of viral peptides, with special interest in peptide sequences that exactly matched protein sequences of the novel 2019 SARS COV-2 virus. To that end, we used the ViPR database, which lists the results of T cell immunogenicity and MHC peptide-binding assays for both HLA-I and HLA-II alleles for viral pathogen epitopes. We used all assays of Coronaviridae family viruses with human hosts from ViPR as our validation dataset. Assays that did not have an associated four-digit HLA allele or were associated with an allele our models did not support were omitted.

For HLA-I, within the validation dataset there were a total of 4,445 unique peptide-HLA allele pairs that were assayed for MHC-binding, using variations of: 1) cellular MHC or purified MHC; 2) a direct or competitive assay; and 3) measurement by fluorescence or radioactivity. Two additional peptide-MHC allele pairs were confirmed via X-ray crystallography. Depending on the study from which the data was collected, peptide-MHC allele pairs were either defined in ViPR simply as “Negative” and “Positive” for binding, or with a more granular scale of positivity: Low, Intermediate, and High. We assigned peptide-MHC allele pairs with multiple measurements with the highest MHC-binding detected across the replicates (see Methods).

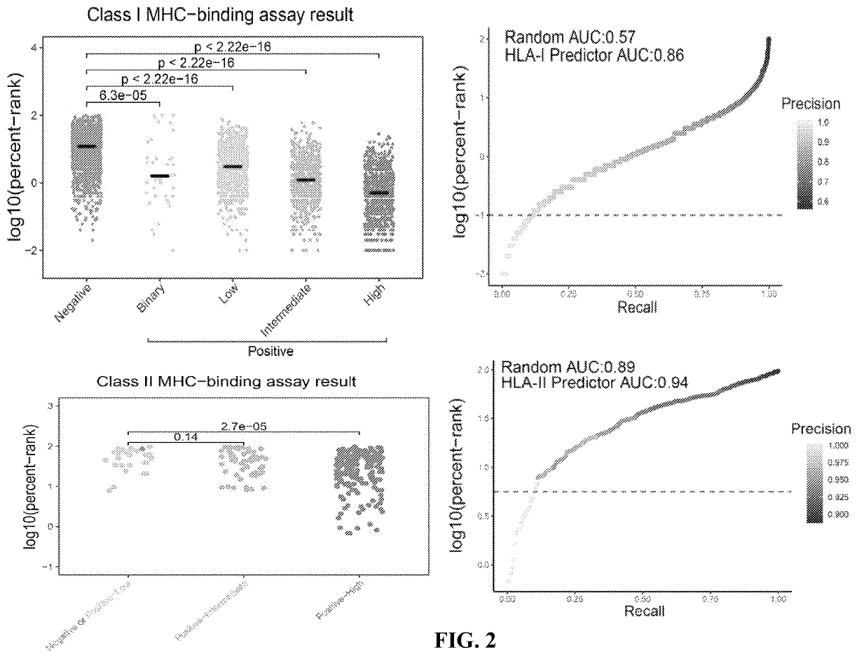

We then applied our HLA-I binding predictor to the peptide-MHC allele pairs in the validation dataset and compared the computed HLA-I percent ranks of these pairs with the reported MHC-binding assay results. A low percent rank value corresponds to high likelihood of binding (e.g., a peptide with a percent rank of 1% scores amongst the top 1% in a reference set of random peptides). The percent ranks of peptide-MHC allele pairs that had a binary “Positive” result in the MHC-binding assay were significantly lower than pairs with a “Negative” result. Further, in the more granular positive results, stronger assay results (low<intermediate<high) were associated with increasingly lower percent ranks (FIG. 2, Upper Left). In addition, the two peptide-MHC alleles that were confirmed by X-ray crystallography were predicted as very likely binders, with low percent rank scores of 0.07% and 0.30% r Although our HLA-I binding predictor was initially built with the purpose of supporting neoantigen prediction in cancer, this analysis shows that it can be successfully applied to coronavirus proteomes. We evaluated our predictor by performing a Precision-Recall analysis, demonstrating the tradeoff between accurate calling of positive binders and the fraction of true binders that are detected (FIG. 2, Lower Left).

Assays of T cell reactivity (e.g., interferon-gamma ELISpots, tetramers), which are stricter measures for T cell immunogenicity to epitopes, were performed in significantly lower numbers compared with MHC-binding assays. For HLA-I, the overlap between peptide-MHC allele pairs for which we had a prediction (supported alleles) and pairs with a reported T cell assay consisted of only 32 pairs, of which 23 had a positive result. We did not detect differences in the percent ranks across the positive and negative groups, however sample sizes are extremely small (data not shown). In addition, for HLA-I epitopes, the validation dataset only contained T cell assay results for peptide-MHC allele pairs that had a positive result in a binding assay, suggesting a highly biased pool of epitopes selected for testing, as also reflected in the high rate of positive T cell assay results. Indeed, the high rate of positive MHC binding assays compared to what would be expected for completely randomly selected peptides also implies that peptides expected to bind based on prediction or prior data were prioritized for testing (or negative results were under-reported). This underlying bias in peptides assayed is important to keep in mind in evaluating the binding predictor performance on this validation dataset. An even more dramatic difference in scores for positives versus negatives could be expected had random peptides been selected for testing.

In addition to the identification of targets for CD8+ T cells, we have recently demonstrated the ability to predict HLA-II binders, allowing us to target CD4+ T cell responses which could be harnessed for 2019 SARS CoV-2 vaccines. These CD4+ responses can potentially bolster both T cell immunity and enhance humoral immunity.

In a similar fashion to the HLA-I analysis, we scored all Coronaviridae family peptide-MHC allele pairs with supported HLA-II alleles in ViPR using our HLA-II binding predictor. There were 259 unique peptide-MHC allele pairs assayed by MHC-binding assays in the ViPR validation dataset for HLA-II. As before, we compared their percent rank with their reported ‘best’ (in the case of multiple measurements) MHC-binding assay result. This comparison could not be performed with the “Negative” pairs as an independent group since there was only one negative result in the validation dataset for HLA-II. The low negative counts may be due to under-reporting of negative assay results or biased selection of the peptides to be assayed. Therefore, we merged the “Negative” and “Positive-Low” groups into one group and compared their percent ranks with either the “Positive-Intermediate” or the “Positive-High” groups (FIG. 2, Upper Right). This analysis revealed a trend similar to that observed with HLA-I predictions, indicating that stronger MHC-binding assay results are associated with a lower predicted percent rank for HLA-II binders, as we expect for a robust predictor. We also evaluated our HLA-II binding predictor by performing a Precision-Recall analysis (FIG. 2, Lower Right). The area under the Precision-Recall curve (AUC) indicated only a small advantage to our predictor over a random guess, which is explained by the heavy bias towards peptides with positive HLA-II binding assay results. Similar to the HLA-I T cell assays, there were too few recorded HLA-II T cell assays in our validation dataset to determine percent rank differences between peptide-HLA II allele pairs testing positive and negative. Together, these findings further corroborate the validity of our epitope predictors, as peptide-MHC allele pairs with positive results in binding assays consistently have lower percent ranks (better scores) by both our HLA-I and HLA-II MHC-binding predictors.

### Epitope Prediction for SARS-CoV-2

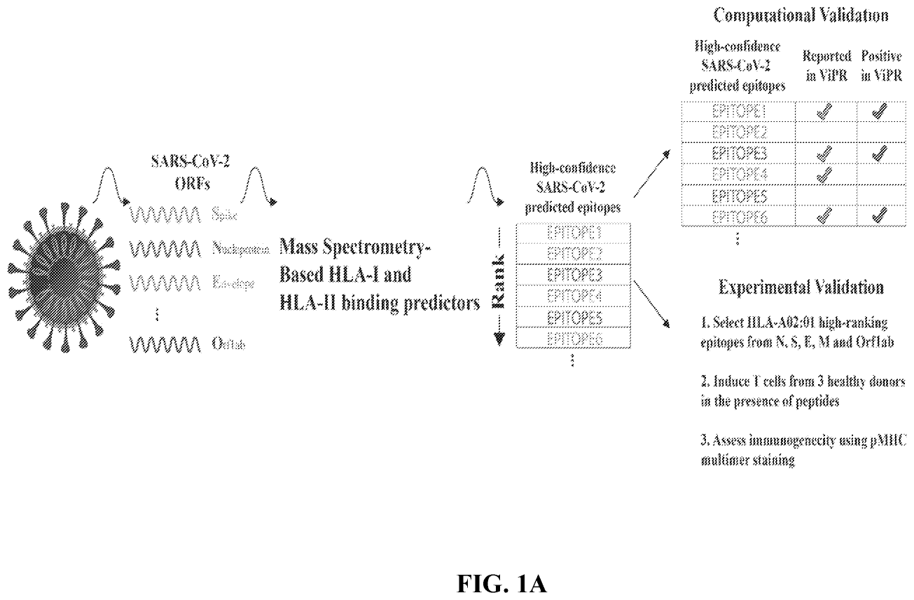

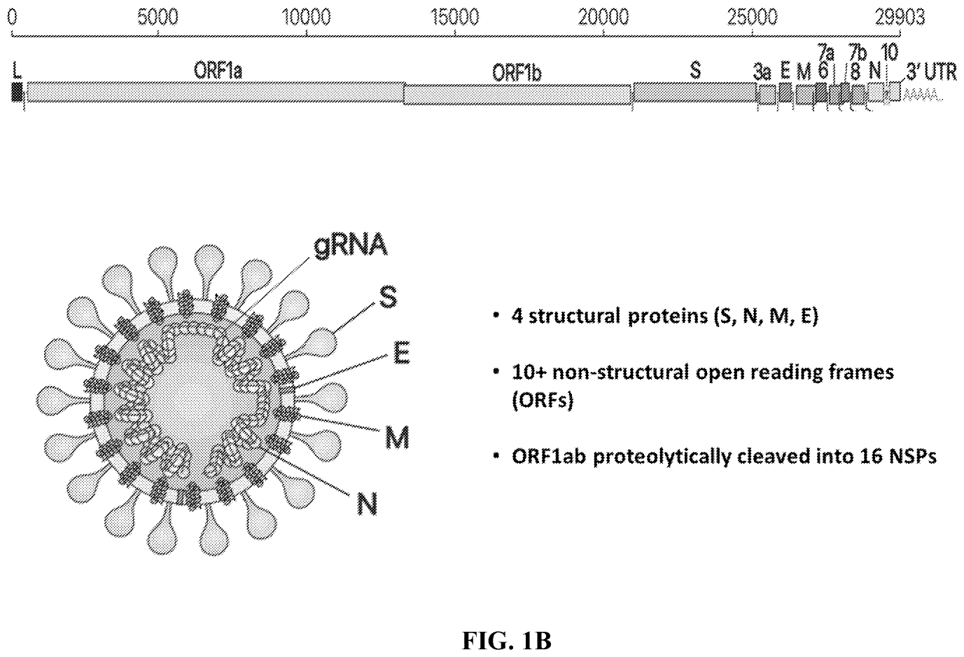

We harnessed our MS-based HLA binding prediction ability to identify the peptides most relevant to the generation of SARS CoV-2 T cell responses. We first performed the analysis for HLA-I peptide binding and computed the likelihood of each peptide of lengths 8-12 amino-acids from the 13 SARS CoV-2 ORFs to bind to any HLA-I allele in our database. We then calculated the percent rank of each peptide-MHC allele pair by comparing their binding scores to those of a set of reference peptides; putative binders were identified as sequences predicted to bind to a given allele with a percent rank of 1% or lower (FIG. 1).

By this metric, we detected a total of 11,897 unique SARS CoV-2 peptides that were predicted to bind at least one HLA-I allele. 16 of these peptides overlapped with a subsequence of the human proteome and were marked for considerations of potential autoimmunity.

Unlike HLA-I, which has a closed binding groove that constrains bound peptide lengths to approximately 8 to 12 amino acids, peptides binding HLA-II have a wider length distribution (up to 30 amino acids or even longer) since the HLA-II binding groove is open at both ends. Peptides bind with a 9 amino acid subsequence (termed the binding core) occupying the HLA-II binding groove, with any flanking sequence overhanging the edges of the molecule. We consider a group of peptides that differ in the flanking regions but share a common binding core as a single epitope. Using the HLA-II predictor we identified 3,372 unique binding-cores that are predicted to bind at least one HLA-II allele with a percent rank score of 1% or lower (Table 5). The majority of predicted peptide-MHC allele pairs are from ORF1a and ORF1ab, primarily driven by the length of these ORFs. In addition, ORF1a and ORF1ab have very similar sequences, with over 18,000 identical binding peptide-HLA-I allele pairs predicted for both ORFs. We therefore opted to exclude redundant predictions and only reported unique pairs (see * in Table 5). Similarly, all HLA-II predicted epitopes from ORF1a were covered by those reported for ORF1ab.

To test the validity of the SARS CoV-2 predicted peptide-HLA pairs, we looked for peptide sequences in the Coronaviridae portion of the ViPR database which exactly matched SARS CoV-2 peptide sequences (FIG. 2D). A total of 374 HLA-I peptide-MHC allele pairs from SARS CoV-2 had both a percent rank lower than 1% by our predictor and were found in the HLA-I MHC-binding validation dataset. Strikingly, of these HLA-I peptide-MHC allele pairs, 333 (89%) had a positive assay result. As a comparison, we also tested for overlap between epitopes predicted to have low likelihood of MHC-binding (percent rank 50% or higher) and the validation dataset. 37 peptide-MHC allele pairs overlapped between these sets, of which 36 (97.2%) had a negative assay result, as predicted. Further, we sought to determine whether our highly predicted 2019 SARS CoV-2 peptide-HLA-I allele pairs (percent rank lower than 1%) would be validated by reported T cell assay results. Despite the significantly smaller number of peptide-MHC allele pairs that were tested for T cell reactivity in the validation dataset, 10 assayed pairs were also highly predicted by our HLA-I binding predictor. Nine out of these 10 (90%) predicted pairs had a positive result to the T cell assay. No low-scoring pairs (percent rank of 50% or above) were reported in the validation dataset. These findings demonstrate the validity of our prediction for peptide-HLA-I allele pairs for SARS CoV-2 epitopes. Notably, while our algorithms are not trained on T cell reactivity data and are aimed at peptide-MHC binding, for the few examples in ViPR for which T cell reactivity assay results were reported, we were able to show our highly-scoring peptide-MHC allele pairs are indeed immunogenic in the vast majority of cases.

For HLA-II peptide-MHC allele pairs, only a single HLA-II peptide-MHC allele pair had both a percent rank lower than 1% and was reported in the validation dataset; this single pair (from the envelope protein) had a “Positive-High” assay result.

TABLE 5

| | | Peptide | | | | | Binding-core |
|---|---|---|---|---|---|---|---|
| | Length | HLA-I pair | Reported | Assay: | Assay: | Percent- | and HLA-II |
| Protein | (AA) | count | in ViPR | Negative | Positive | positive | pair count |
| envelope protein (E) | 75 | 556 | 34 | 3 | 31 | 91.2 | 29 |
| membrane | 222 | 1236 | 41 | 0 | 41 | 100.0 | 68 |
| glycoprotein (M) | | | | | | | |
| nucleocapsid | 419 | 1054 | 40 | 9 | 31 | 77.5 | 107 |
| phosphoprotein (N) | | | | | | | |
| ORF1a | 4405 | 14* | 0 | 0 | 0 | NA | 0* |
| polyprotein* | | | | | | | |
| ORF1ab | 7096 | 28965 | 0 | 0 | 0 | 0NA | 2516 |
| polyprotein | | | | | | | |
| ORF3a protein | 275 | 1408 | 127 | 11 | 116 | 91.3 | 94 |
| ORF6 protein | 61 | 322 | 0 | 0 | 0 | NA | 23 |
| ORF7a protein | 121 | 642 | 3 | 0 | 3 | 100.0 | 28 |
| ORF7b | 43 | 327 | 18 | 1 | 7 | 87.5 | 2 |
| ORF8 protein | 121 | 449 | 20 | 2 | 18 | 90.0 | 27 |
| ORF9b protein** | 97 | 453 | 6 | 1 | 5 | 83.3 | 37 |
| ORF10 protein | 38 | 258 | 0 | 0 | 0 | NA | 4 |
| surface glycoprotein | 1273 | 4686 | 95 | 14 | 81 | 85.3 | 437 |

*peptides unique to orf1a (not found in orf1ab).
**annotated in UniProt.

### Immunogenicity of HLA-A02:01-Predicted 2019 SARS CoV-2 Epitopes

The MS-based binding prediction algorithms used predict the likelihood of an epitope to be presented by a specific HLA allele, but do not directly predict the ability of a T cell receptor to recognize the epitope presented by the MHC molecule. Due to the process of central tolerance, which deletes T cells that could cross-react with peptides from self-antigens, not every epitope that is a strong MHC binder will elicit a T cell response. Therefore, there is a need to further validate high affinity MHC binding peptides in T cell assays (FIG. 1). To address the immunogenicity of a subset of highly predicted MHC binding peptides, we synthesized 23 highly predicted HLA-A02:01 binding epitopes from each of the following SARS CoV-2 proteins: S, M, N, E, and ORF1ab. Of these highly predicted SARS CoV-2 epitopes, five sequences were positive in ViPR for MHC binding or T cell reactivity in association with HLA-A02:01. Pools of these peptides were cultured with PBMCs from three human donors, and the predicted epitopes were considered immunogenic if they elicited a T cell response as detected by binding to pMHC multimers for HLA-A02:01 in at least one of three donors.

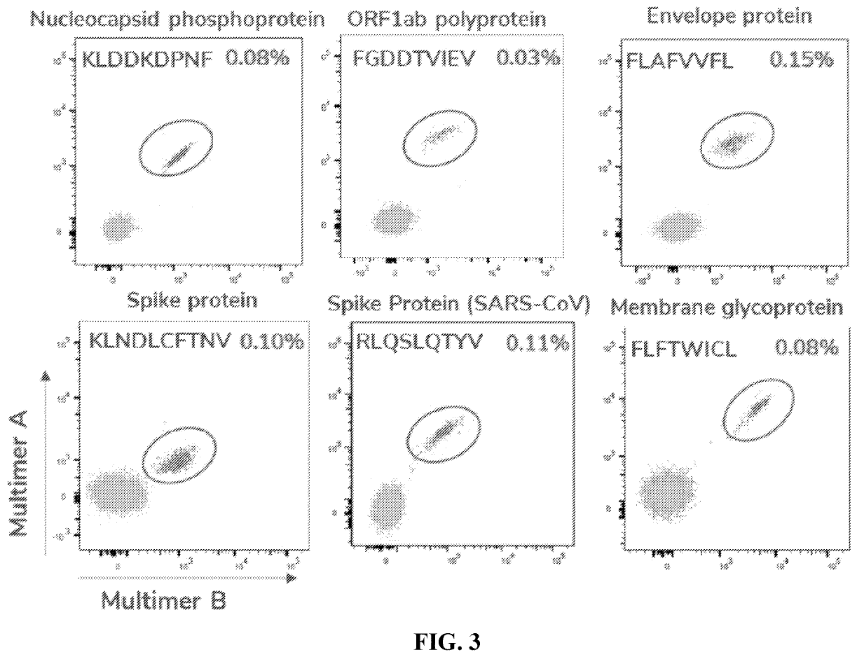

As shown in FIG. 3, CD8+ T cell responses were validated in at least one donor for 11 of the 23 highly predicted epitopes (Table 7 and FIG. 3). Of the five epitopes previously reported as either immunogenic or strong binders in ViPR, three were confirmed to elicit a CD8+ T cell response, confirming that our binding predictor can identify epitopes that are immunogenic. In addition, we were able to identify eight novel epitopes not previously reported in ViPR that were recognized by specific CD8+ T cells in donor PBMCs. The responses were generally robust, with nine of the 11 epitopes positive in our assay were recognized by specific CD8+ T cell responses in at least two donors (Table 7), and encouragingly, every ORF from 2019 SARS CoV-2 that was assayed had at least one peptide that led to a T cell response (Table 7). Taken together, these data show that many novel 2019 SARS CoV-2 epitopes that were predicted to be strong binders from MHC-I binding predictor were found to be immunogenic.

### Population Coverage of Peptides Predicted to Bind Multiple HLA-I and HLA-II Alleles

The concordance between the validation dataset and the highly predicted peptide-MHC allele pairs provided herein indicate that the HLA binding predictors significantly expand the list of predicted MHC binding peptides from the ORFs of 2019 SARS-CoV-2. Next, peptides from the M, N, and S proteins were prioritized, that were predicted to provide broad coverage for the USA, European (EU) and Asian Pacific Islander (API) populations based on the prevalence of MHC alleles in these populations. It was found that a subset of the peptides was predicted to bind a broad set of either HLA-I or HLA-II alleles. For each protein, it was determined that a small number of peptide sequences provide saturating coverage for the USA, European, and Asian Pacific Islander populations, with >99% population coverage achieved with selected 8-12mer epitopes for HLA-I, and >95% population coverage achieved with selected 25mer sequences for HLA-II, respectively (FIG. 4B). Even if the generous assumption that all peptide-MHC allele pairs for which a given peptide scores in the top 1% are indeed immunogenic is not fully upheld, this finding could facilitate the design of a parsimonious, broadly effective vaccine to induce broad T cell immunity.

Table 6 shows the top HLA-I predicted binders from each of the three SARS CoV-2 proteins: spike, nucleocapsid and membrane with the broadest cumulative allele coverage. The table provides the peptide sequence, its rank, the 2019 SARS CoV-2 protein it is derived from, the alleles the peptide is predicted to bind to and the cumulative HLA-I coverage for USA, European (EUR), and Asian Pacific Islander (API) populations for all peptides up to this rank.

Table 7 shows the top HLA-II predicted binders from each of the three 2019 SARS CoV-2 proteins: spike, nucleocapsid and membrane For each 25mer, the table provides the rank, the peptide sequence, the 2019 SARS CoV-2 protein it is derived from, the cumulative alleles that are covered by all 25mers up to this rank, and the associated USA, European (EUR), and Asian Pacific Islander (API) population coverage. Note that it is not the case that any of these 25mers, or their binding subsequences, are found as subsequences within the human proteome.

TABLE 6

| Rank | Peptide | P | Cumulative Alleles | USA | EUR | API |
|---|---|---|---|---|---|---|
| 1 | YQPYR | S | HLA-A02:07; HLA-B08:01; HLA-B13:02; HLA- | 0.9701 | 0.978332 | 0.973286 |
| | VVVL | | B14:02; HLA-B15:01; HLA-B15:03; HLA-B15:09; | | | |
| | | | HLA-B37:01; HLA-B39:01; HLA-B42:01; HLA- | | | |
| | | | B48:01; HLA-B52:01; HLA-C01:02; HLA-C02:02; | | | |
| | | | HLA-C03:04; HLA-C04:01; HLA-C05:01; HLA- | | | |
| | | | C06:02; HLA-C07:01; HLA-C07:02; HLA-C08:01; | | | |
| | | | HLA-C12:02; HLA-C14:02; HLA-C14:03; HLA- | | | |
| | | | C15:02 | | | |
| 2 | FVFLVL | S | HLA-A02:01; HLA-A02:03; HLA-A02:04; HLA- | 0.9977 | 0.998821 | 0.997513 |
| | LPL | | A02:07; HLA-A03:01; HLA-A68:02; HLA-B08:01; | | | |
| | | | HLA-B13:02; HLA-B14:02; HLA-B15:01; HLA- | | | |
| | | | B15:03; HLA-B15:09; HLA-B35:01; HLA-B35:03; | | | |
| | | | HLA-B37:01; HLA-B39:01; HLA-B42:01; HLA- | | | |
| | | | B48:01; HLA-B51:01; HLA-B52:01; HLA-B54:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:01; HLA-C08:02; HLA-C12:02; HLA-C14:02; | | | |
| | | | HLA-C14:03; HLA-C15:02; HLA-C17:01 | | | |
| 3 | CVADY | S | HLA-A01:01; HLA-A02:01; HLA-A02:03; HLA- | 0.9996 | 0.999899 | 0.999257 |
| | SVLY | | A02:04; HLA-A02:07; HLA-A03:01; HLA-A11:01; | | | |
| | | | HLA-A25:01; HLA-A26:01; HLA-A29:02; HLA- | | | |
| | | | A30:01; HLA-A30:02; HLA-A32:01; HLA-A36:01; | | | |
| | | | HLA-A68:01; HLA-A68:02; HLA-B08:01; HLA- | | | |
| | | | B13:02; HLA-B14:02; HLA-B15:01; HLA-B15:03; | | | |
| | | | HLA-B15:09; HLA-B35:01; HLA-B35:03; HLA- | | | |
| | | | B37:01; HLA-B39:01; HLA-B42:01; HLA-B48:01; | | | |
| | | | HLA-B51:01; HLA-B52:01; HLA-B54:01; HLA- | | | |
| | | | B58:01; HLA-C01:02; HLA-C02:02; HLA-C03:02; | | | |
| | | | HLA-C03:03; HLA-C03:04; HLA-C04:01; HLA- | | | |
| | | | C05:01; HLA-C06:02; HLA-C07:01; HLA-C07:02; | | | |
| | | | HLA-C08:01; HLA-C08:02; HLA-C12:02; HLA- | | | |
| | | | C14:02; HLA-C14:03; HLA-C15:02; HLA-C17:01 | | | |
| 4 | QPTESI | S | HLA-A01:01; HLA-A02:01; HLA-A02:03; HLA- | 0.9999 | 0.999991 | 0.999754 |
| | VRF | | A02:04; HLA-A02:07; HLA-A03:01; HLA-A11:01; | | | |
| | | | HLA-A24:02; HLA-A25:01; HLA-A26:01; HLA- | | | |
| | | | A29:02; HLA-A30:01; HLA-A30:02; HLA-A32:01; | | | |
| | | | HLA-A36:01; HLA-A68:01; HLA-A68:02; HLA- | | | |
| | | | B07:02; HLA-B08:01; HLA-B13:02; HLA-B14:02; | | | |
| | | | HLA-B15:01; HLA-B15:03; HLA-B15:09; HLA- | | | |
| | | | B18:01; HLA-B35:01; HLA-B35:03; HLA-B37:01; | | | |
| | | | HLA-B38:01; HLA-B39:01; HLA-B42:01; HLA- | | | |
| | | | B48:01; HLA-B51:01; HLA-B52:01; HLA-B53:01; | | | |
| | | | HLA-B54:01; HLA-B58:01; HLA-B81:01; HLA- | | | |
| | | | C01:02; HLA-C02:02; HLA-C03:02; HLA-C03:03; | | | |
| | | | HLA-C03:04; HLA-C04:01; HLA-C05:01; HLA- | | | |
| | | | C06:02; HLA-C07:01; HLA-C07:02; HLA-C08:01; | | | |
| | | | HLA-C08:02; HLA-C12:02; HLA-C14:02; HLA- | | | |
| | | | C14:03; HLA-C15:02; HLA-C17:01 | | | |
| 5 | AEVQID | S | HLA-A01:01; HLA-A02:01; HLA-A02:03; HLA- | 0.9999 | 0.999999 | 0.999879 |
| | RLI | | A02:04; HLA-A02:07; HLA-A03:01; HLA-A11:01; | | | |
| | | | HLA-A24:02; HLA-A25:01; HLA-A26:01; HLA- | | | |
| | | | A29:02; HLA-A30:01; HLA-A30:02; HLA-A32:01; | | | |
| | | | HLA-A36:01; HLA-A68:01; HLA-A68:02; HLA- | | | |
| | | | B07:02; HLA-B08:01; HLA-B13:02; HLA-B14:02; | | | |
| | | | HLA-B15:01; HLA-B15:03; HLA-B15:09; HLA- | | | |
| | | | B18:01; HLA-B35:01; HLA-B35:03; HLA-B37:01; | | | |
| | | | HLA-B38:01; HLA-B39:01; HLA-B40:01; HLA- | | | |
| | | | B40:02; HLA-B41:01; HLA-B42:01; HLA-B44:02; | | | |
| | | | HLA-B44:03; HLA-B45:01; HLA-B48:01; HLA- | | | |
| | | | B49:01; HLA-B50:01; HLA-B51:01; HLA-B52:01; | | | |
| | | | HLA-B53:01; HLA-B54:01; HLA-B58:01; HLA- | | | |
| | | | B58:02; HLA-B81:01; HLA-C01:02; HLA-C02:02; | | | |
| | | | HLA-C03:02; HLA-C03:03; HLA-C03:04; HLA- | | | |
| | | | C04:01; HLA-C05:01; HLA-C06:02; HLA-C07:01; | | | |
| | | | HLA-C07:02; HLA-C08:01; HLA-C08:02; HLA- | | | |
| | | | C12:02; HLA-C14:02; HLA-C14:03; HLA-C15:02; | | | |
| | | | HLA-C17:01 | | | |
| 6 | QSAPHG | S | HLA-A01:01; HLA-A02:01; HLA-A02:03; HLA- | 0.9999 | 1 | 0.999962 |
| | VVF | | A02:04; HLA-A02:07; HLA-A03:01; HLA-A11:01; | | | |
| | | | HLA-A24:02; HLA-A25:01; HLA-A26:01; HLA- | | | |
| | | | A29:02; HLA-A30:01; HLA-A30:02; HLA-A32:01; | | | |
| | | | HLA-A36:01; HLA-A68:01; HLA-A68:02; HLA- | | | |
| | | | B07:02; HLA-B08:01; HLA-B13:02; HLA-B14:02; | | | |
| | | | HLA-B15:01; HLA-B15:03; HLA-B15:09; HLA- | | | |
| | | | B18:01; HLA-B35:01; HLA-B35:03; HLA-B37:01; | | | |
| | | | HLA-B38:01; HLA-B39:01; HLA-B40:01; HLA- | | | |
| | | | B40:02; HLA-B41:01; HLA-B42:01; HLA-B44:02; | | | |
| | | | HLA-B44:03; HLA-B45:01; HLA-B46:01; HLA- | | | |
| | | | B48:01; HLA-B49:01; HLA-B50:01; HLA-B51:01; | | | |
| | | | HLA-B52:01; HLA-B53:01; HLA-B54:01; HLA- | | | |
| | | | B57:01; HLA-B57:03; HLA-B58:01; HLA-B58:02; | | | |
| | | | HLA-B81:01; HLA-C01:02; HLA-C02:02; HLA- | | | |
| | | | C03:02; HLA-C03:03; HLA-C03:04; HLA-C04:01; | | | |
| | | | HLA-C05:01; HLA-C06:02; HLA-C07:01; HLA- | | | |
| | | | C07:02; HLA-C08:01; HLA-C08:02; HLA-C12:02; | | | |
| | | | HLA-C12:03; HLA-C14:02; HLA-C14:03; HLA- | | | |
| | | | C15:02; HLA-C17:01 | | | |
| 7 | VYYPD | S | HLA-A01:01; HLA-A02:01; HLA-A02:03; HLA- | 0.9999 | 1 | 0.999988 |
| | KVFR | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B35:01; HLA-B35:03; HLA- | | | |
| | | | B37:01; HLA-B38:01; HLA-B39:01; HLA-B40:01; | | | |
| | | | HLA-B40:02; HLA-B41:01; HLA-B42:01; HLA- | | | |
| | | | B44:02; HLA-B44:03; HLA-B45:01; HLA-B46:01; | | | |
| | | | HLA-B48:01; HLA-B49:01; HLA-B50:01; HLA- | | | |
| | | | B51:01; HLA-B52:01; HLA-B53:01; HLA-B54:01; | | | |
| | | | HLA-B57:01; HLA-B57:03; HLA-B58:01; HLA- | | | |
| | | | B58:02; HLA-B81:01; HLA-C01:02; HLA-C02:02; | | | |
| | | | HLA-C03:02; HLA-C03:03; HLA-C03:04; HLA- | | | |
| | | | C04:01; HLA-C05:01; HLA-C06:02; HLA-C07:01; | | | |
| | | | HLA-C07:02; HLA-C08:01; HLA-C08:02; HLA- | | | |
| | | | C12:02; HLA-C12:03; HLA-C14:02; HLA-C14:03; | | | |
| | | | HLA-C15:02; HLA-C17:01 | | | |
| 8 | RRARSV | S | HLA-A01:01; HLA-A02:01; HLA-A02:03; HLA- | 0.9999 | 1 | 0.999989 |
| | ASQ | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B37:01; HLA-B38:01; HLA-B39:01; | | | |
| | | | HLA-B40:01; HLA-B40:02; HLA-B41:01; HLA- | | | |
| | | | B42:01; HLA-B44:02; HLA-B44:03; HLA-B45:01; | | | |
| | | | HLA-B46:01; HLA-B48:01; HLA-B49:01; HLA- | | | |
| | | | B50:01; HLA-B51:01; HLA-B52:01; HLA-B53:01; | | | |
| | | | HLA-B54:01; HLA-B57:01; HLA-B57:03; HLA- | | | |
| | | | B58:01; HLA-B58:02; HLA-B81:01; HLA-C01:02; | | | |
| | | | HLA-C02:02; HLA-C03:02; HLA-C03:03; HLA- | | | |
| | | | C03:04; HLA-C04:01; HLA-C05:01; HLA-C06:02; | | | |
| | | | HLA-C07:01; HLA-C07:02; HLA-C08:01; HLA- | | | |
| | | | C08:02; HLA-C12:02; HLA-C12:03; HLA-C14:02; | | | |
| | | | HLA-C14:03; HLA-C15:02; HLA-C17:01 | | | |
| 9 | CPFGEV | S | HLA-A01:01; HLA-A02:01; HLA-A02:03; HLA- | 0.9999 | 1 | 0.999989 |
| | FNA | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B37:01; HLA-B38:01; HLA-B39:01; | | | |
| | | | HLA-B40:01; HLA-B40:02; HLA-B41:01; HLA- | | | |
| | | | B42:01; HLA-B44:02; HLA-B44:03; HLA-B45:01; | | | |
| | | | HLA-B46:01; HLA-B48:01; HLA-B49:01; HLA- | | | |
| | | | B50:01; HLA-B51:01; HLA-B52:01; HLA-B53:01; | | | |
| | | | HLA-B54:01; HLA-B55:01; HLA-B57:01; HLA- | | | |
| | | | B57:03; HLA-B58:01; HLA-B58:02; HLA-B81:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:01; HLA-C08:02; HLA-C12:02; HLA-C12:03; | | | |
| | | | HLA-C14:02; HLA-C14:03; HLA-C15:02; HLA- | | | |
| | | | C17:01 | | | |
| 10 | SAPHGV | S | HLA-A01:01; HLA-A02:01; HLA-A02:03; HLA- | 0.9999 | 1 | 0.999989 |
| | VFL | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B37:01; HLA-B38:01; HLA-B39:01; | | | |
| | | | HLA-B40:01; HLA-B40:02; HLA-B41:01; HLA- | | | |
| | | | B42:01; HLA-B44:02; HLA-B44:03; HLA-B45:01; | | | |
| | | | HLA-B46:01; HLA-B48:01; HLA-B49:01; HLA- | | | |
| | | | B50:01; HLA-B51:01; HLA-B52:01; HLA-B53:01; | | | |
| | | | HLA-B54:01; HLA-B55:01; HLA-B57:01; HLA- | | | |
| | | | B57:03; HLA-B58:01; HLA-B58:02; HLA-B81:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:01; HLA-C08:02; HLA-C12:02; HLA-C12:03; | | | |
| | | | HLA-C14:02; HLA-C14:03; HLA-C15:02; HLA- | | | |
| | | | C17:01 | | | |
| 1 | FNPETN | M | HLA-A02:07; HLA-C01:02; HLA-C03:04; HLA- | 0.8460 | 0.8464 | 0.888243 |
| | ILL | | C04:01; HLA-C05:01; HLA-C06:02; HLA-C07:02; | | | |
| | | | HLA-C08:01; HLA-C08:02; HLA-C14:02; HLA- | | | |
| | | | C17:01 | | | |
| 2 | SSSDNI | M | HLA-A01:01; HLA-A02:03; HLA-A02:04; HLA- | 0.9949 | 0.9981 | 0.987775 |
| | ALL | | A02:07; HLA-A68:01; HLA-A68:02; HLA-B15:09; | | | |
| | | | HLA-B38:01; HLA-B39:01; HLA-B48:01; HLA- | | | |
| | | | B57:03; HLA-B81:01; HLA-C01:02; HLA-C02:02; | | | |
| | | | HLA-C03:03; HLA-C03:04; HLA-C04:01; HLA- | | | |
| | | | C05:01; HLA-C06:02; HLA-C07:01; HLA-C07:02; | | | |
| | | | HLA-C08:01; HLA-C08:02; HLA-C12:02; HLA- | | | |
| | | | C12:03; HLA-C14:02; HLA-C15:02; HLA-C17:01 | | | |
| 3 | KLLEQ | M | HLA-A01:01; HLA-A02:01; HLA-A02:03; HLA- | 0.9987 | 0.999727 | 0.992178 |
| | WNLV | | A02:04; HLA-A02:07; HLA-A03:01; HLA-A30:01; | | | |
| | | | HLA-A32:01; HLA-A68:01; HLA-A68:02; HLA- | | | |
| | | | B13:02; HLA-B15:09; HLA-B38:01; HLA-B39:01; | | | |
| | | | HLA-B48:01; HLA-B57:03; HLA-B81:01; HLA- | | | |
| | | | C01:02; HLA-C02:02; HLA-C03:03; HLA-C03:04; | | | |
| | | | HLA-C04:01; HLA-C05:01; HLA-C06:02; HLA- | | | |
| | | | C07:01; HLA-C07:02; HLA-C08:01; HLA-C08:02; | | | |
| | | | HLA-C12:02; HLA-C12:03; HLA-C14:02; HLA- | | | |
| | | | C15:02; HLA-C17:01 | | | |
| 4 | LWLLW | M | HLA-A01:01; HLA-A02:01; HLA-A02:03; HLA- | 0.9997 | 0.99994 | 0.999326 |
| | PVTL | | A02:04; HLA-A02:07; HLA-A03:01; HLA-A23:01; | | | |
| | | | HLA-A24:02; HLA-A30:01; HLA-A32:01; HLA- | | | |
| | | | A68:01; HLA-A68:02; HLA-B08:01; HLA-B13:02; | | | |
| | | | HLA-B14:02; HLA-B15:09; HLA-B35:01; HLA- | | | |
| | | | B38:01; HLA-B39:01; HLA-B48:01; HLA-B51:01; | | | |
| | | | HLA-B54:01; HLA-B57:03; HLA-B58:02; HLA- | | | |
| | | | B81:01; HLA-C01:02; HLA-C02:02; HLA-C03:02; | | | |
| | | | HLA-C03:03; HLA-C03:04; HLA-C04:01; HLA- | | | |
| | | | C05:01; HLA-C06:02; HLA-C07:01; HLA-C07:02; | | | |
| | | | HLA-C08:01; HLA-C08:02; HLA-C12:02; HLA- | | | |
| | | | C12:03; HLA-C14:02; HLA-C14:03; HLA-C15:02; | | | |
| | | | HLA-C17:01 | | | |
| 5 | SELVIG | M | HLA-A01:01; HLA-A02:01; HLA-A02:03; HLA- | 0.9998 | 0.999979 | 0.999644 |
| | AVI | | A02:04; HLA-A02:07; HLA-A03:01; HLA-A23:01; | | | |
| | | | HLA-A24:02; HLA-A30:01; HLA-A32:01; HLA- | | | |
| | | | A68:01; HLA-A68:02; HLA-B08:01; HLA-B13:02; | | | |
| | | | HLA-B14:02; HLA-B15:09; HLA-B18:01; HLA- | | | |
| | | | B35:01; HLA-B38:01; HLA-B39:01; HLA-B40:01; | | | |
| | | | HLA-B40:02; HLA-B41:01; HLA-B44:02; HLA- | | | |
| | | | B44:03; HLA-B45:01; HLA-B48:01; HLA-B49:01; | | | |
| | | | HLA-B51:01; HLA-B52:01; HLA-B54:01; HLA- | | | |
| | | | B57:03; HLA-B58:02; HLA-B81:01; HLA-C01:02; | | | |
| | | | HLA-C02:02; HLA-C03:02; HLA-C03:03; HLA- | | | |
| | | | C03:04; HLA-C04:01; HLA-C05:01; HLA-C06:02; | | | |
| | | | HLA-C07:01; HLA-C07:02; HLA-C08:01; HLA- | | | |
| | | | C08:02; HLA-C12:02; HLA-C12:03; HLA-C14:02; | | | |
| | | | HLA-C14:03; HLA-C15:02; HLA-C17:01 | | | |
| 6 | ATSRTL | M | HLA-A01:01; HLA-A02:01; HLA-A02:03; HLA- | 0.9999 | 0.999999 | 0.999913 |
| | SYY | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A32:01; HLA-A36:01; HLA-A68:01; HLA- | | | |
| | | | A68:02; HLA-B08:01; HLA-B13:02; HLA-B14:02; | | | |
| | | | HLA-B15:09; HLA-B18:01; HLA-B35:01; HLA- | | | |
| | | | B38:01; HLA-B39:01; HLA-B40:01; HLA-B40:02; | | | |
| | | | HLA-B41:01; HLA-B44:02; HLA-B44:03; HLA- | | | |
| | | | B45:01; HLA-B48:01; HLA-B49:01; HLA-B51:01; | | | |
| | | | HLA-B52:01; HLA-B54:01; HLA-B57:01; HLA- | | | |
| | | | B57:03; HLA-B58:01; HLA-B58:02; HLA-B81:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:01; HLA-C08:02; HLA-C12:02; HLA-C12:03; | | | |
| | | | HLA-C14:02; HLA-C14:03; HLA-C15:02; HLA- | | | |
| | | | C17:01 | | | |
| 7 | LPKEIT | M | HLA-A01:01; HLA-A02:01; HLA-A02:03; HLA- | 0.9999 | 1 | 0.99995 |
| | VAT | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A32:01; HLA-A36:01; HLA-A68:01; HLA- | | | |
| | | | A68:02; HLA-B07:02; HLA-B08:01; HLA-B13:02; | | | |
| | | | HLA-B14:02; HLA-B15:09; HLA-B18:01; HLA- | | | |
| | | | B35:01; HLA-B35:03; HLA-B38:01; HLA-B39:01; | | | |
| | | | HLA-B40:01; HLA-B40:02; HLA-B41:01; HLA- | | | |
| | | | B42:01; HLA-B44:02; HLA-B44:03; HLA-B45:01; | | | |
| | | | HLA-B46:01; HLA-B48:01; HLA-B49:01; HLA- | | | |
| | | | B51:01; HLA-B52:01; HLA-B54:01; HLA-B55:01; | | | |
| | | | HLA-B57:01; HLA-B57:03; HLA-B58:01; HLA- | | | |
| | | | B58:02; HLA-B81:01; HLA-C01:02; HLA-C02:02; | | | |
| | | | HLA-C03:02; HLA-C03:03; HLA-C03:04; HLA- | | | |
| | | | C04:01; HLA-C05:01; HLA-C06:02; HLA-C07:01; | | | |
| | | | HLA-C07:02; HLA-C08:01; HLA-C08:02; HLA- | | | |
| | | | C12:02; HLA-C12:03; HLA-C14:02; HLA-C14:03; | | | |
| | | | HLA-C15:02; HLA-C17:01 | | | |
| 8 | EQWNL | M | HLA-A01:01; HLA-A02:01; HLA-A02:03; HLA- | 0.9999 | 1 | 0.999961 |
| | VIGF | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A32:01; HLA-A36:01; HLA-A68:01; HLA- | | | |
| | | | A68:02; HLA-B07:02; HLA-B08:01; HLA-B13:02; | | | |
| | | | HLA-B14:02; HLA-B15:01; HLA-B15:03; HLA- | | | |
| | | | B15:09; HLA-B18:01; HLA-B27:05; HLA-B35:01; | | | |
| | | | HLA-B35:03; HLA-B38:01; HLA-B39:01; HLA- | | | |
| | | | B40:01; HLA-B40:02; HLA-B41:01; HLA-B42:01; | | | |
| | | | HLA-B44:02; HLA-B44:03; HLA-B45:01; HLA- | | | |
| | | | B46:01; HLA-B48:01; HLA-B49:01; HLA-B51:01; | | | |
| | | | HLA-B52:01; HLA-B54:01; HLA-B55:01; HLA- | | | |
| | | | B57:01; HLA-B57:03; HLA-B58:01; HLA-B58:02; | | | |
| | | | HLA-B81:01; HLA-C01:02; HLA-C02:02; HLA- | | | |
| | | | C03:02; HLA-C03:03; HLA-C03:04; HLA-C04:01; | | | |
| | | | HLA-C05:01; HLA-C06:02; HLA-C07:01; HLA- | | | |
| | | | C07:02; HLA-C08:01; HLA-C08:02; HLA-C12:02; | | | |
| | | | HLA-C12:03; HLA-C14:02; HLA-C14:03; HLA- | | | |
| | | | C15:02; HLA-C17:01 | | | |
| 9 | FVLAA | M | HLA-A01:01; HLA-A02:01; HLA-A02:03; HLA- | 0.9999 | 1 | 0.999987 |
| | VYR | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B38:01; HLA-B39:01; HLA-B40:01; | | | |
| | | | HLA-B40:02; HLA-B41:01; HLA-B42:01; HLA- | | | |
| | | | B44:02; HLA-B44:03; HLA-B45:01; HLA-B46:01; | | | |
| | | | HLA-B48:01; HLA-B49:01; HLA-B51:01; HLA- | | | |
| | | | B52:01; HLA-B54:01; HLA-B55:01; HLA-B57:01; | | | |
| | | | HLA-B57:03; HLA-B58:01; HLA-B58:02; HLA- | | | |
| | | | B81:01; HLA-C01:02; HLA-C02:02; HLA-C03:02; | | | |
| | | | HLA-C03:03; HLA-C03:04; HLA-C04:01; HLA- | | | |
| | | | C05:01; HLA-C06:02; HLA-C07:01; HLA-C07:02; | | | |
| | | | HLA-C08:01; HLA-C08:02; HLA-C12:02; HLA- | | | |
| | | | C12:03; HLA-C14:02; HLA-C14:03; HLA-C15:02; | | | |
| | | | HLA-C17:01 | | | |
| 10 | SELVIG | M | HLA-A01:01; HLA-A02:01; HLA-A02:03; HLA- | 0.9999 | 1 | 0.999989 |
| | AV | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B37:01; HLA-B38:01; HLA-B39:01; | | | |
| | | | HLA-B40:01; HLA-B40:02; HLA-B41:01; HLA- | | | |
| | | | B42:01; HLA-B44:02; HLA-B44:03; HLA-B45:01; | | | |
| | | | HLA-B46:01; HLA-B48:01; HLA-B49:01; HLA- | | | |
| | | | B50:01; HLA-B51:01; HLA-B52:01; HLA-B54:01; | | | |
| | | | HLA-B55:01; HLA-B57:01; HLA-B57:03; HLA- | | | |
| | | | B58:01; HLA-B58:02; HLA-B81:01; HLA-C01:02; | | | |
| | | | HLA-C02:02; HLA-C03:02; HLA-C03:03; HLA- | | | |
| | | | C03:04; HLA-C04:01; HLA-C05:01; HLA-C06:02; | | | |
| | | | HLA-C07:01; HLA-C07:02; HLA-C08:01; HLA- | | | |
| | | | C08:02; HLA-C12:02; HLA-C12:03; HLA-C14:02; | | | |
| | | | HLA-C14:03; HLA-C15:02; HLA-C17:01 | | | |
| 1 | FAPSAS | NP | HLA-B46:01; HLA-B51:01; HLA-C01:02; HLA- | 0.8892 | 0.893062 | 0.909801 |
| | AF | | C02:02; HLA-C03:02; HLA-C03:03; HLA-C03:04; | | | |
| | | | HLA-C04:01; HLA-C05:01; HLA-C07:02; HLA- | | | |
| | | | C08:02; HLA-C12:03; HLA-C14:02; HLA-C14:03; | | | |
| | | | HLA-C17:01 | | | |
| 2 | LLLDRL | NP | HLA-A02:01; HLA-A02:03; HLA-A02:04; HLA- | 0.9837 | 0.98958 | 0.964647 |
| | NQL | | A02:07; HLA-A03:01; HLA-A23:01; HLA-A32:01; | | | |
| | | | HLA-B08:01; HLA-B14:02; HLA-B46:01; HLA- | | | |
| | | | B51:01; HLA-C01:02; HLA-C02:02; HLA-C03:02; | | | |
| | | | HLA-C03:03; HLA-C03:04; HLA-C04:01; HLA- | | | |
| | | | C05:01; HLA-C06:02; HLA-C07:02; HLA-C08:02; | | | |
| | | | HLA-C12:03; HLA-C14:02; HLA-C14:03; HLA- | | | |
| | | | C17:01 | | | |
| 3 | SSPDDQ | NP | HLA-A01:01; HLA-A02:01; HLA-A02:03; HLA- | 0.9973 | 0.999047 | 0.989434 |
| | IGY | | A02:04; HLA-A02:07; HLA-A03:01; HLA-A11:01; | | | |
| | | | HLA-A23:01; HLA-A25:01; HLA-A26:01; HLA- | | | |
| | | | A29:02; HLA-A30:01; HLA-A30:02; HLA-A32:01; | | | |
| | | | HLA-A36:01; HLA-B08:01; HLA-B14:02; HLA- | | | |
| | | | B15:01; HLA-B15:03; HLA-B46:01; HLA-B51:01; | | | |
| | | | HLA-B57:01; HLA-B57:03; HLA-B58:01; HLA- | | | |
| | | | C01:02; HLA-C02:02; HLA-C03:02; HLA-C03:03; | | | |
| | | | HLA-C03:04; HLA-C04:01; HLA-C05:01; HLA- | | | |
| | | | C06:02; HLA-C07:02; HLA-C08:02; HLA-C12:03; | | | |
| | | | HLA-C14:02; HLA-C14:03; HLA-C17:01 | | | |
| 4 | TPSGTW | NP | HLA-A01:01; HLA-A02:01; HLA-A02:03; HLA- | 0.9997 | 0.999954 | 0.994134 |
| | LTY | | A02:04; HLA-A02:07; HLA-A03:01; HLA-A11:01; | | | |
| | | | HLA-A23:01; HLA-A25:01; HLA-A26:01; HLA- | | | |
| | | | A29:02; HLA-A30:01; HLA-A30:02; HLA-A32:01; | | | |
| | | | HLA-A36:01; HLA-B07:02; HLA-B08:01; HLA- | | | |
| | | | B14:02; HLA-B15:01; HLA-B15:03; HLA-B35:01; | | | |
| | | | HLA-B35:03; HLA-B42:01; HLA-B46:01; HLA- | | | |
| | | | B51:01; HLA-B53:01; HLA-B55:01; HLA-B57:01; | | | |
| | | | HLA-B57:03; HLA-B58:01; HLA-C01:02; HLA- | | | |
| | | | C02:02; HLA-C03:02; HLA-C03:03; HLA-C03:04; | | | |
| | | | HLA-C04:01; HLA-C05:01; HLA-C06:02; HLA- | | | |
| | | | C07:01; HLA-C07:02; HLA-C08:02; HLA-C12:03; | | | |
| | | | HLA-C14:02; HLA-C14:03; HLA-C17:01 | | | |
| 5 | MEVTPS | NP | HLA-A01:01; HLA-A02:01; HLA-A02:03; HLA- | 0.9999 | 0.999992 | 0.996542 |
| | GTW | | A02:04; HLA-A02:07; HLA-A03:01; HLA-A11:01; | | | |
| | | | HLA-A23:01; HLA-A25:01; HLA-A26:01; HLA- | | | |
| | | | A29:02; HLA-A30:01; HLA-A30:02; HLA-A32:01; | | | |
| | | | HLA-A36:01; HLA-B07:02; HLA-B08:01; HLA- | | | |
| | | | B14:02; HLA-B15:01; HLA-B15:03; HLA-B18:01; | | | |
| | | | HLA-B35:01; HLA-B35:03; HLA-B40:01; HLA- | | | |
| | | | B42:01; HLA-B44:02; HLA-B44:03; HLA-B46:01; | | | |
| | | | HLA-B49:01; HLA-B50:01; HLA-B51:01; HLA- | | | |
| | | | B53:01; HLA-B55:01; HLA-B57:01; HLA-B57:03; | | | |
| | | | HLA-B58:01; HLA-C01:02; HLA-C02:02; HLA- | | | |
| | | | C03:02; HLA-C03:03; HLA-C03:04; HLA-C04:01; | | | |
| | | | HLA-C05:01; HLA-C06:02; HLA-C07:01; HLA- | | | |
| | | | C07:02; HLA-C08:02; HLA-C12:03; HLA-C14:02; | | | |
| | | | HLA-C14:03; HLA-C17:01 | | | |
| 6 | KHWPQI | NP | HLA-A01:01; HLA-A02:01; HLA-A02:03; HLA- | 0.9999 | 0.999999 | 0.998895 |
| | AQF | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A32:01; HLA-A36:01; HLA-B07:02; HLA- | | | |
| | | | B08:01; HLA-B14:02; HLA-B15:01; HLA-B15:03; | | | |
| | | | HLA-B15:09; HLA-B18:01; HLA-B35:01; HLA- | | | |
| | | | B35:03; HLA-B37:01; HLA-B38:01; HLA-B39:01; | | | |
| | | | HLA-B40:01; HLA-B42:01; HLA-B44:02; HLA- | | | |
| | | | B44:03; HLA-B46:01; HLA-B48:01; HLA-B49:01; | | | |
| | | | HLA-B50:01; HLA-B51:01; HLA-B53:01; HLA- | | | |
| | | | B55:01; HLA-B57:01; HLA-B57:03; HLA-B58:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:02; HLA-C12:03; HLA-C14:02; HLA-C14:03; | | | |
| | | | HLA-C17:01 | | | |
| 7 | ASAFFG | NP | HLA-A01:01; HLA-A02:01; HLA-A02:03; HLA- | 0.9999 | 1 | 0.999685 |
| | MSR | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A74:01; HLA-B07:02; | | | |
| | | | HLA-B08:01; HLA-B14:02; HLA-B15:01; HLA- | | | |
| | | | B15:03; HLA-B15:09; HLA-B18:01; HLA-B35:01; | | | |
| | | | HLA-B35:03; HLA-B37:01; HLA-B38:01; HLA- | | | |
| | | | B39:01; HLA-B40:01; HLA-B42:01; HLA-B44:02; | | | |
| | | | HLA-B44:03; HLA-B46:01; HLA-B48:01; HLA- | | | |
| | | | B49:01; HLA-B50:01; HLA-B51:01; HLA-B53:01; | | | |
| | | | HLA-B55:01; HLA-B57:01; HLA-B57:03; HLA- | | | |
| | | | B58:01; HLA-C01:02; HLA-C02:02; HLA-C03:02; | | | |
| | | | HLA-C03:03; HLA-C03:04; HLA-C04:01; HLA- | | | |
| | | | C05:01; HLA-C06:02; HLA-C07:01; HLA-C07:02; | | | |
| | | | HLA-C08:02; HLA-C12:03; HLA-C14:02; HLA- | | | |
| | | | C14:03; HLA-C17:01 | | | |
| 8 | DAALA | NP | HLA-A01:01; HLA-A02:01; HLA-A02:03; HLA- | 0.9999 | 1 | 0.999977 |
| | LLLL | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B14:02; HLA- | | | |
| | | | B15:01; HLA-B15:03; HLA-B15:09; HLA-B18:01; | | | |
| | | | HLA-B35:01; HLA-B35:03; HLA-B37:01; HLA- | | | |
| | | | B38:01; HLA-B39:01; HLA-B40:01; HLA-B42:01; | | | |
| | | | HLA-B44:02; HLA-B44:03; HLA-B46:01; HLA- | | | |
| | | | B48:01; HLA-B49:01; HLA-B50:01; HLA-B51:01; | | | |
| | | | HLA-B53:01; HLA-B55:01; HLA-B57:01; HLA- | | | |
| | | | B57:03; HLA-B58:01; HLA-C01:02; HLA-C02:02; | | | |
| | | | HLA-C03:02; HLA-C03:03; HLA-C03:04; HLA- | | | |
| | | | C04:01; HLA-C05:01; HLA-C06:02; HLA-C07:01; | | | |
| | | | HLA-C07:02; HLA-C08:01; HLA-C08:02; HLA- | | | |
| | | | C12:02; HLA-C12:03; HLA-C14:02; HLA-C14:03; | | | |
| | | | HLA-C15:02; HLA-C17:01 | | | |
| 9 | RQKKQ | NP | HLA-A01:01; HLA-A02:01; HLA-A02:03; HLA- | 0.9999 | 1 | 0.999983 |
| | QTV | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B35:01; HLA-B35:03; HLA- | | | |
| | | | B37:01; HLA-B38:01; HLA-B39:01; HLA-B40:01; | | | |
| | | | HLA-B42:01; HLA-B44:02; HLA-B44:03; HLA- | | | |
| | | | B46:01; HLA-B48:01; HLA-B49:01; HLA-B50:01; | | | |
| | | | HLA-B51:01; HLA-B52:01; HLA-B53:01; HLA- | | | |
| | | | B55:01; HLA-B57:01; HLA-B57:03; HLA-B58:01; | | | |
| | | | HLA-C01:02; HLA-C02:02; HLA-C03:02; HLA- | | | |
| | | | C03:03; HLA-C03:04; HLA-C04:01; HLA-C05:01; | | | |
| | | | HLA-C06:02; HLA-C07:01; HLA-C07:02; HLA- | | | |
| | | | C08:01; HLA-C08:02; HLA-C12:02; HLA-C12:03; | | | |
| | | | HLA-C14:02; HLA-C14:03; HLA-C15:02; HLA- | | | |
| | | | C17:01 | | | |
| 10 | SRIGME | NP | HLA-A01:01; HLA-A02:01; HLA-A02:03; HLA- | 0.9999 | 1 | 0.999984 |
| | VTP | | A02:04; HLA-A02:07; HLA-A03:01; HLA-A11:01; | | | |
| | | | HLA-A23:01; HLA-A24:02; HLA-A25:01; HLA- | | | |
| | | | A26:01; HLA-A29:02; HLA-A30:01; HLA-A30:02; | | | |
| | | | HLA-A31:01; HLA-A32:01; HLA-A33:03; HLA- | | | |
| | | | A36:01; HLA-A68:01; HLA-A68:02; HLA-A74:01; | | | |
| | | | HLA-B07:02; HLA-B08:01; HLA-B13:02; HLA- | | | |
| | | | B14:02; HLA-B15:01; HLA-B15:03; HLA-B15:09; | | | |
| | | | HLA-B18:01; HLA-B27:05; HLA-B35:01; HLA- | | | |
| | | | B35:03; HLA-B37:01; HLA-B38:01; HLA-B39:01; | | | |
| | | | HLA-B40:01; HLA-B42:01; HLA-B44:02; HLA- | | | |
| | | | B44:03; HLA-B46:01; HLA-B48:01; HLA-B49:01; | | | |
| | | | HLA-B50:01; HLA-B51:01; HLA-B52:01; HLA- | | | |
| | | | B53:01; HLA-B55:01; HLA-B57:01; HLA-B57:03; | | | |
| | | | HLA-B58:01; HLA-C01:02; HLA-C02:02; HLA- | | | |
| | | | C03:02; HLA-C03:03; HLA-C03:04; HLA-C04:01; | | | |
| | | | HLA-C05:01; HLA-C06:02; HLA-C07:01; HLA- | | | |
| | | | C07:02; HLA-C08:01; HLA-C08:02; HLA-C12:02; | | | |
| | | | HLA-C12:03; HLA-C14:02; HLA-C14:03; HLA- | | | |
| | | | C15:02; HLA-C17:01 | | | |

Index for Table: P, viral protein; S, Surface glycoprotein; M, membrane glycoprotein; NP, nucleocapsid phosphoprotein.

TABLE 7

| S1 | Peptide | P | Cumulative Alleles | USA | EUR | API |
|---|---|---|---|---|---|---|
| 1 | TPPIKD | S | DRB1_1001; DQB1-0602_DQA1-0102; DRB1_0401; | 0.973835 | 0.972746 | 0.757235 |
| | FGGFN | | DRB3_0101; DPB1-0401_DPA1-0103; DPB1-0402_DPA1- | | | |
| | FSQILP | | 0103; DRB1_1303; DQB1-0303_DQA1-0201; DRB1_0405; | | | |
| | DPSKPS | | DRB1_0901; DRB1_0411; DRB1_0404; DPB1-0201_DPA1- | | | |
| | KR | | 0103; DQB1-0302_DQA1-0301; DPB1-0101_DPA1-0202 | | | |
| 2 | EIDRLN | S | DRB4_0103; DPB1-1401_DPA1-0201; DPB1-0301_DPA1- | 0.997968 | 0.997785 | 0.982844 |
| | EVAKN | | 0103; DRB3_0301; DPB1-0901_DPA1-0201; DPB1- | | | |
| | LNESLI | | 1701_DPA1-0201; DPB1-1001_DPA1-0201; DQB1- | | | |
| | DLQEL | | 0604_DQA1-0102; DQB1-0502_DQA1-0102; DPB1- | | | |
| | GKY | | 0501_DPA1-0202; DRB1_1001; DQB1-0602_DQA1-0102; | | | |
| | | | DRB1_0401; DRB3_0101; DPB1-0401_DPA1-0103; DPB1- | | | |
| | | | 0402_DPA1-0103; DRB1_1303; DQB1-0303_DQA1-0201; | | | |
| | | | DRB1_0405; DRB1_0901; DRB1_0411; DRB1_0404; DPB1- | | | |
| | | | 0201_DPA1-0103; DQB1-0302_DQA1-0301; DPB1- | | | |
| | | | 0101_DPA1-0202 | | | |
| 3 | EKGIY | S | DRB1_0701; DRB1_0101; DRB1_1502; DRB1_0406; DPB1- | 0.99924 | 0.999337 | 0.993125 |
| | QTSNF | | 0202_DPA1-0103; DRB1_1501; DRB1_0302; DRB1_1001; | | | |
| | RVQPT | | DQB1-0602_DQA1-0102; DRB1_0401; DRB3_0101; DPB1- | | | |
| | ESIVRF | | 0401_DPA1-0103; DPB1-0402_DPA1-0103; DRB1_1303; | | | |
| | PNIT | | DQB1-0303_DQA1-0201; DRB1_0405; DRB1_0901; | | | |
| | | | DRB1_0411; DRB1_0404; DPB1-0201_DPA1-0103; DQB1- | | | |
| | | | 0302_DQA1-0301; DPB1-0101_DPA1-0202; DRB4_0103; | | | |
| | | | DPB1-1401_DPA1-0201; DPB1-0301_DPA1-0103; | | | |
| | | | DRB3_0301; DPB1-0901_DPA1-0201; DPB1-1701_DPA1- | | | |
| | | | 0201; DPB1-1001_DPA1-0201; DQB1-0604_DQA1-0102; | | | |
| | | | DQB1-0502_DQA1-0102; DPB1-0501_DPA1-0202 | | | |
| 4 | NPVLP | S | DQB1-0301_DQA1-0505; DQB1-0201_DQA1-0501; | 0.999858 | 0.999877 | 0.995801 |
| | FNDGV | | DRB1_0403; DRB1_1401; DRB1_1405; DQB1-0202_DQA1- | | | |
| | YFAST | | 0201; DRB1_1001; DQB1-0602_DQA1-0102; DRB1_0401; | | | |
| | EKSNII | | DRB3_0101; DPB1-0401_DPA1-0103; DPB1-0402_DPA1- | | | |
| | RGWI | | 0103; DRB1_1303; DQB1-0303_DQA1-0201; DRB1_0405; | | | |
| | | | DRB1_0901; DRB1_0411; DRB1_0404; DPB1-0201_DPA1- | | | |
| | | | 0103; DQB1-0302_DQA1-0301; DPB1-0101_DPA1-0202; | | | |
| | | | DRB4_0103; DPB1-1401_DPA1-0201; DPB1-0301_DPA1- | | | |
| | | | 0103; DRB3_0301; DPB1-0901_DPA1-0201; DPB1- | | | |
| | | | 1701_DPA1-0201; DPB1-1001_DPA1-0201; DQB1- | | | |
| | | | 0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302 | | | |
| 5 | VFNAT | S | DPB1-1301_DPA1-0201; DRB5_0101; DRB5_0102; | 0.999973 | 0.999978 | 0.997479 |
| | RFASV | | DRB1_1301; DRB1_1102; DRB1_1101; DRB1_1001; DQB1- | | | |
| | YAWN | | 0602_DQA1-0102; DRB1_0401; DRB3_0101; DPB1- | | | |
| | RKRISN | | 0401_DPA1-0103; DPB1-0402_DPA1-0103; DRB1_1303; | | | |
| | CVADY | | DQB1-0303_DQA1-0201; DRB1_0405; DRB1_0901; | | | |
| | | | DRB1_0411; DRB1_0404; DPB1-0201_DPA1-0103; DQB1- | | | |
| | | | 0302_DQA1-0301; DPB1-0101_DPA1-0202; DRB4_0103; | | | |
| | | | DPB1-1401_DPA1-0201; DPB1-0301_DPA1-0103; | | | |
| | | | DRB3_0301; DPB1-0901_DPA1-0201; DPB1-1701_DPA1- | | | |
| | | | 0201; DPB1-1001_DPA1-0201; DQB1-0604_DQA1-0102; | | | |
| | | | DQB1-0502_DQA1-0102; DPB1-0501_DPA1-0202; | | | |
| | | | DRB1_0701; DRB1_0101; DRB1_1502; DRB1_0406; DPB1- | | | |
| | | | 0202_DPA1-0103; DRB1_1501; DRB1_0302; DQB1- | | | |
| | | | 0301_DQA1-0505; DQB1-0201_DQA1-0501; DRB1_0403; | | | |
| | | | DRB1_1401; DRB1_1405; DQB1-0202_DQA1-0201 | | | |
| 6 | ISNCV | S | DRB1_0802; DRB1_0301; DRB3_0201; DRB3_0202; | 0.999997 | 0.999998 | 0.99835 |
| | ADYSV | | DRB1_1001; DQB1-0602_DQA1-0102; DRB1_0401; | | | |
| | LYNSA | | DRB3_0101; DPB1-0401_DPA1-0103; DPB1-0402_DPA1- | | | |
| | SFSTFK | | 0103; DRB1_1303; DQB1-0303_DQA1-0201; DRB1_0405; | | | |
| | CYGV | | DRB1_0901; DRB1_0411; DRB1_0404; DPB1-0201_DPA1- | | | |
| | | | 0103; DQB1-0302_DQA1-0301; DPB1-0101_DPA1-0202; | | | |
| | | | DRB4_0103; DPB1-1401_DPA1-0201; DPB1-0301_DPA1- | | | |
| | | | 0103; DRB3_0301; DPB1-0901_DPA1-0201; DPB1- | | | |
| | | | 1701_DPA1-0201; DPB1-1001_DPA1-0201; DQB1- | | | |
| | | | 0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302; DQB1-0301_DQA1-0505; DQB1-0201_DQA1- | | | |
| | | | 0501; DRB1_0403; DRB1_1401; DRB1_1405; DQB1- | | | |
| | | | 0202_DQA1-0201; DPB1-1301_DPA1-0201; DRB5_0101; | | | |
| | | | DRB5_0102; DRB1_1301; DRB1_1102; DRB1_1101 | | | |
| 7 | IGIVNN | S | DQB1-0201_DQA1-0201; DQB1-0603_DQA1-0103; | 0.999999 | 1 | 0.999149 |
| | TVYDP | | DRB1_0803; DQB1-0501_DQA1-0101; DRB1_1001; DQB1- | | | |
| | LQPEL | | 0602_DQA1-0102; DRB1_0401; DRB3_0101; DPB1- | | | |
| | DSFKE | | 0401_DPA1-0103; DPB1-0402_DPA1-0103; DRB1_1303; | | | |
| | ELDK | | DQB1-0303_DQA1-0201; DRB1_0405; DRB1_0901; | | | |
| | | | DRB1_0411; DRB1_0404; DPB1-0201_DPA1-0103; DQB1- | | | |
| | | | 0302_DQA1-0301; DPB1-0101_DPA1-0202; DRB4_0103; | | | |
| | | | DPB1-1401_DPA1-0201; DPB1-0301_DPA1-0103; | | | |
| | | | DRB3_0301; DPB1-0901_DPA1-0201; DPB1-1701_DPA1- | | | |
| | | | 0201; DPB1-1001_DPA1-0201; DQB1-0604_DQA1-0102; | | | |
| | | | DQB1-0502_DQA1-0102; DPB1-0501_DPA1-0202; | | | |
| | | | DRB1_0701; DRB1_0101; DRB1_1502; DRB1_0406; DPB1- | | | |
| | | | 0202_DPA1-0103; DRB1_1501; DRB1_0302; DQB1- | | | |
| | | | 0301_DQA1-0505; DQB1-0201_DQA1-0501; DRB1_0403; | | | |
| | | | DRB1_1401; DRB1_1405; DQB1-0202_DQA1-0201; DPB1- | | | |
| | | | 1301_DPA1-0201; DRB5_0101; DRB5_0102; DRB1_1301; | | | |
| | | | DRB1_1102; DRB1_1101; DRB1_0802; DRB1_0301; | | | |
| | | | DRB3_0201; DRB3_0202 | | | |
| 8 | DEVRQ | S | DQB1-0301_DQA1-0601; DQB1-0601_DQA1-0103; DQB1- | 1 | 1 | 0.999546 |
| | IAPGQT | | 0301_DQA1-0303; DRB1_1001; DQB1-0602_DQA1-0102; | | | |
| | GKIAD | | DRB1_0401; DRB3_0101; DPB1-0401_DPA1-0103; DPB1- | | | |
| | YNYKL | | 0402_DPA1-0103; DRB1_1303; DQB1-0303_DQA1-0201; | | | |
| | PDDF | | DRB1_0405; DRB1_0901; DRB1_0411; DRB1_0404; DPB1- | | | |
| | | | 0201_DPA1-0103; DQB1-0302_DQA1-0301; DPB1- | | | |
| | | | 0101_DPA1-0202; DRB4_0103; DPB1-1401_DPA1-0201; | | | |
| | | | DPB1-0301_DPA1-0103; DRB3_0301; DPB1-0901_DPA1- | | | |
| | | | 0201; DPB1-1701_DPA1-0201; DPB1-1001_DPA1-0201; | | | |
| | | | DQB1-0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302; DQB1-0301_DQA1-0505; DQB1-0201_DQA1- | | | |
| | | | 0501; DRB1_0403; DRB1_1401; DRB1_1405; DQB1- | | | |
| | | | 0202_DQA1-0201; DPB1-1301_DPA1-0201; DRB5_0101; | | | |
| | | | DRB5_0102; DRB1_1301; DRB1_1102; DRB1_1101; | | | |
| | | | DRB1_0802; DRB1_0301; DRB3_0201; DRB3_0202; DQB1- | | | |
| | | | 0201_DQA1-0201; DQB1-0603_DQA1-0103; DRB1_0803; | | | |
| | | | DQB1-0501_DQA1-0101 | | | |
| 9 | GDSTE | S | DRB1_0102; DQB1-0303_DQA1-0301; DRB1_1602; | 1 | 1 | 0.999816 |
| | CSNLL | | DRB1_1503; DRB1_1001; DQB1-0602_DQA1-0102; | | | |
| | LQYGS | | DRB1_0401; DRB3_0101; DPB1-0401_DPA1-0103; DPB1- | | | |
| | FCTQL | | 0402_DPA1-0103; DRB1_1303; DQB1-0303_DQA1-0201; | | | |
| | NRALT | | DRB1_0405; DRB1_0901; DRB1_0411; DRB1_0404; DPB1- | | | |
| | | | 0201_DPA1-0103; DQB1-0302_DQA1-0301; DPB1- | | | |
| | | | 0101_DPA1-0202; DRB4_0103; DPB1-1401_DPA1-0201; | | | |
| | | | DPB1-0301_DPA1-0103; DRB3_0301; DPB1-0901_DPA1- | | | |
| | | | 0201; DPB1-1701_DPA1-0201; DPB1-1001_DPA1-0201; | | | |
| | | | DQB1-0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302; DQB1-0301_DQA1-0505; DQB1-0201_DQA1- | | | |
| | | | 0501; DRB1_0403; DRB1_1401; DRB1_1405; DQB1- | | | |
| | | | 0202_DQA1-0201; DPB1-1301_DPA1-0201; DRB5_0101; | | | |
| | | | DRB5_0102; DRB1_1301; DRB1_1102; DRB1_1101; | | | |
| | | | DRB1_0802; DRB1_0301; DRB3_0201; DRB3_0202; DQB1- | | | |
| | | | 0201_DQA1-0201; DQB1-0603_DQA1-0103; DRB1_0803; | | | |
| | | | DQB1-0501_DQA1-0101; DQB1-0301_DQA1-0601; DQB1- | | | |
| | | | 0601_DQA1-0103; DQB1-0301_DQA1-0303 | | | |
| 10 | KNIDG | S | DRB1_1302; DRB1_0801; DRB1_0407; DRB1_1001; DQB1- | 1 | 1 | 0.999882 |
| | YFKIYS | | 0602_DQA1-0102; DRB1_0401; DRB3_0101; DPB1- | | | |
| | KHTPIN | | 0401_DPA1-0103; DPB1-0402_DPA1-0103; DRB1_1303; | | | |
| | LVRDL | | DQB1-0303_DQA1-0201; DRB1_0405; DRB1_0901; | | | |
| | PQG | | DRB1_0411; DRB1_0404; DPB1-0201_DPA1-0103; DQB1- | | | |
| | | | 0302_DQA1-0301; DPB1-0101_DPA1-0202; DRB4_0103; | | | |
| | | | DPB1-1401_DPA1-0201; DPB1-0301_DPA1-0103; | | | |
| | | | DRB3_0301; DPB1-0901_DPA1-0201; DPB1-1701_DPA1- | | | |
| | | | 0201; DPB1-1001_DPA1-0201; DQB1-0604_DQA1-0102; | | | |
| | | | DQB1-0502_DQA1-0102; DPB1-0501_DPA1-0202; | | | |
| | | | DRB1_0701; DRB1_0101; DRB1_1502; DRB1_0406; DPB1- | | | |
| | | | 0202_DPA1-0103; DRB1_1501; DRB1_0302; DQB1- | | | |
| | | | 0301_DQA1-0505; DQB1-0201_DQA1-0501; DRB1_0403; | | | |
| | | | DRB1_1401; DRB1_1405; DQB1-0202_DQA1-0201; DPB1- | | | |
| | | | 1301_DPA1-0201; DRB5_0101; DRB5_0102; DRB1_1301; | | | |
| | | | DRB1_1102; DRB1_1101; DRB1_0802; DRB1_0301; | | | |
| | | | DRB3_0201; DRB3_0202; DQB1-0201_DQA1-0201; DQB1- | | | |
| | | | 0603_DQA1-0103; DRB1_0803; DQB1-0501_DQA1-0101; | | | |
| | | | DQB1-0301_DQA1-0601; DQB1-0601_DQA1-0103; DQB1- | | | |
| | | | 0301_DQA1-0303; DRB1_0102; DQB1-0303_DQA1-0301; | | | |
| | | | DRB1_1602; DRB1_1503 | | | |
| 11 | HADQL | S | DPB1-0601_DPA1-0103; DQB1-0401_DQA1-0301; 1 | 1 | 1 | 0.999926 |
| | TPTWR | | DRB1_0804; DRB1_1104; DRB1_1001; DQB1-0602_DQA1- | | | |
| | VYSTG | | 0102; DRB1_0401; DRB3_0101; DPB1-0401_DPA1-0103; | | | |
| | SNVFQ | | DPB1-0402_DPA1-0103; DRB1_1303; DQB1-0303_DQA1- | | | |
| | TRAGC | | 0201; DRB1_0405; DRB1_0901; DRB1_0411; DRB1_0404; | | | |
| | | | DPB1-0201_DPA1-0103; DQB1-0302_DQA1-0301; DPB1- | | | |
| | | | 0101_DPA1-0202; DRB4_0103; DPB1-1401_DPA1-0201; | | | |
| | | | DPB1-0301_DPA1-0103; DRB3_0301; DPB1-0901_DPA1- | | | |
| | | | 0201; DPB1-1701_DPA1-0201; DPB1-1001_DPA1-0201; | | | |
| | | | DQB1-0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302; DQB1-0301_DQA1-0505; DQB1-0201_DQA1- | | | |
| | | | 0501; DRB1_0403; DRB1_1401; DRB1_1405; DQB1- | | | |
| | | | 0202_DQA1-0201; DPB1-1301_DPA1-0201; DRB5_0101; | | | |
| | | | DRB5_0102; DRB1_1301; DRB1_1102; DRB1_1101; | | | |
| | | | DRB1_0802; DRB1_0301; DRB3_0201; DRB3_0202; DQB1- | | | |
| | | | 0201_DQA1-0201; DQB1-0603_DQA1-0103; DRB1_0803; | | | |
| | | | DQB1-0501_DQA1-0101; DQB1-0301_DQA1-0601; DQB1- | | | |
| | | | 0601_DQA1-0103; DQB1-0301_DQA1-0303; DRB1_0102; | | | |
| | | | DQB1-0303_DQA1-0301; DRB1_1602; DRB1_1503; | | | |
| | | | DRB1_1302; DRB1_0801; DRB1_0407 | | | |
| 12 | FIAGLI | S | DRB1_1601; DRB1_1202; DPB1-0501_DPA1-0201; | 1 | 1 | 0.999989 |
| | AIVMV | | DRB1_1001; DQB1-0602_DQA1-0102; DRB1_0401; | | | |
| | TIMLC | | DRB3_0101; DPB1-0401_DPA1-0103; DPB1-0402_DPA1- | | | |
| | CMTSC | | 0103; DRB1_1303; DQB1-0303_DQA1-0201; DRB1_0405; | | | |
| | CSCL | | DRB1_0901; DRB1_0411; DRB1_0404; DPB1-0201_DPA1- | | | |
| | | | 0103; DQB1-0302_DQA1-0301; DPB1-0101_DPA1-0202; | | | |
| | | | DRB4_0103; DPB1-1401_DPA1-0201; DPB1-0301_DPA1- | | | |
| | | | 0103; DRB3_0301; DPB1-0901_DPA1-0201; DPB1- | | | |
| | | | 1701_DPA1-0201; DPB1-1001_DPA1-0201; DQB1- | | | |
| | | | 0604_DQA1-0102; DQB1-0502_DQA1-0102; DPB1- | | | |
| | | | 0501_DPA1-0202; DRB1_0701; DRB1_0101; DRB1_1502; | | | |
| | | | DRB1_0406; DPB1-0202_DPA1-0103; DRB1_1501; | | | |
| | | | DRB1_0302; DQB1-0301_DQA1-0505; DQB1-0201_DQA1- | | | |
| | | | 0501; DRB1_0403; DRB1_1401; DRB1_1405; DQB1- | | | |
| | | | 0202_DQA1-0201; DPB1-1301_DPA1-0201; DRB5_0101; | | | |
| | | | DRB5_0102; DRB1_1301; DRB1_1102; DRB1_1101; | | | |
| | | | DRB1_0802; DRB1_0301; DRB3_0201; DRB3_0202; DQB1- | | | |
| | | | 0201_DQA1-0201; DQB1-0603_DQA1-0103; DRB1_0803; | | | |
| | | | DQB1-0501_DQA1-0101; DQB1-0301_DQA1-0601; DQB1- | | | |
| | | | 0601_DQA1-0103;DQB1-0301_DQA1-0303; | | | |
| | | | DRB1_0102;cDQB1-0303_DQA1-0301; DRB1_1602; | | | |
| | | | DRB1_1503; DRB1_1302; DRB1_0801; DRB1_0407; DPB1- | | | |
| | | | 0601_DPA1-0103; DQB1-0401_DQA1-0301; DRB1_0804; | | | |
| | | | DRB1_1104 | | | |
| 1 | KDLPK | M | DQB1-0301_DQA1-0505; DRB1_0102; DQB1-0502_DQA1- | 0.802347 | 0.814186 | 0.610992 |
| | EITVAT | | 0102; DRB1_1601; DRB1_1503; DRB1_0101; DRB1_0403; | | | |
| | SRTLS | | DRB1_0406; DRB5_0102; DQB1-0303_DQA1-0201; | | | |
| | YYKLG | | DRB1_0402; DRB1_1301; DRB1_0404; DRB1_0901; | | | |
| | ASQR | | DRB1_1406; DRB1_1501; DQB1-0202_DQA1-0201; | | | |
| | | | DRB1_1602 | | | |
| 2 | MADSN | M | DRB1_1101; DPB1-0402_DPA1-0103; DRB1_0803; | 0.939299 | 0.942213 | 0.827034 |
| | GTITVE | | DRB1_1104; DQB1-0201_DQA1-0201; DRB1_0804; DQB1- | | | |
| | ELKKL | | 0302_DQA1-0301; DRB1_0801; DRB1_1102; DQB1- | | | |
| | LEQWN | | 0301_DQA1-0303; DQB1-0301_DQA1-0505; DRB1_0102; | | | |
| | LVIG | | DQB1-0502_DQA1-0102; DRB1_1601; DRB1_1503; | | | |
| | | | DRB1_0101; DRB1_0403; DRB1_0406; DRB5_0102; DQB1- | | | |
| | | | 0303_DQA1-0201; DRB1_0402; DRB1_1301; DRB1_0404; | | | |
| | | | DRB1_0901; DRB1_1406; DRB1_1501; DQB1-0202_DQA1- | | | |
| | | | 0201; DRB1_1602 | | | |
| 3 | LWLL | M | DQB1-0602_DQA1-0102; DQB1-0501_DQA1-0101; | 0.987316 | 0.987925 | 0.856152 |
| | WPVTL | | DRB3_0202; DQB1-0301_DQA1-0505; DRB1_0102; DQB1- | | | |
| | ACFVL | | 0502_DQA1-0102; DRB1_1601; DRB1_1503; DRB1_0101; | | | |
| | AAVYR | | DRB1_0403; DRB1_0406; DRB5_0102; DQB1-0303_DQA1- | | | |
| | INWITG | | 0201; DRB1_0402; DRB1_1301; DRB1_0404; DRB1_0901; | | | |
| | | | DRB1_1406; DRB1_1501; DQB1-0202_DQA1-0201; | | | |
| | | | DRB1_1602; DRB1_1101; DPB1-0402_DPA1-0103; | | | |
| | | | DRB1_0803; DRB1_1104; DQB1-0201_DQA1-0201; | | | |
| | | | DRB1_0804; DQB1-0302_DQA1-0301; DRB1_0801; | | | |
| | | | DRB1_1102; DQB1-0301_DQA1-0303 | | | |
| 4 | FIASFR | M | DRB1_0802; DRB1_0701; DRB1_0401; DRB1_0407; DQB1- | 0.99585 | 0.996745 | 0.905042 |
| | LFART | | 0301_DQA1-0505; DRB1_0102; DQB1-0502_DQA1-0102; | | | |
| | RSMWS | | DRB1_1601; DRB1_1503; DRB1_0101; DRB1_0403; | | | |
| | FNPET | | DRB1_0406; DRB5_0102; DQB1-0303_DQA1-0201; | | | |
| | NILL | | DRB1_0402; DRB1_1301; DRB1_0404; DRB1_0901; | | | |
| | | | DRB1_1406; DRB1_1501; DQB1-0202_DQA1-0201; | | | |
| | | | DRB1_1602; DRB1_1101; DPB1-0402_DPA1-0103; | | | |
| | | | DRB1_0803; DRB1_1104; DQB1-0201_DQA1-0201; | | | |
| | | | DRB1_0804; DQB1-0302_DQA1-0301; DRB1_0801; | | | |
| | | | DRB1_1102; DQB1-0301_DQA1-0303; DQB1-0602_DQA1- | | | |
| | | | 0102; DQB1-0501_DQA1-0101; DRB3_0202 | | | |
| 5 | ASQRV | M | DRB1_1302; DQB1-0303_DQA1-0301; DRB1_1502; DQB1- | 0.997289 | 0.997843 | 0.966492 |
| | AGDSG | | 0301_DQA1-0505; DRB1_0102; DQB1-0502_DQA1-0102; | | | |
| | FAAYS | | DRB1_1601; DRB1_1503; DRB1_0101; DRB1_0403; | | | |
| | RYRIG | | DRB1_0406; DRB5_0102; DQB1-0303_DQA1-0201; | | | |
| | NYKLN | | DRB1_0402; DRB1_1301; DRB1_0404; DRB1_0901; | | | |
| | | | DRB1_1406; DRB1_1501; DQB1-0202_DQA1-0201; | | | |
| | | | DRB1_1602; DRB1_1101; DPB1-0402_DPA1-0103; | | | |
| | | | DRB1_0803; DRB1_1104; DQB1-0201_DQA1-0201; | | | |
| | | | DRB1_0804; DQB1-0302_DQA1-0301; DRB1_0801; | | | |
| | | | DRB1_1102; DQB1-0301_DQA1-0303; DQB1-0602_DQA1- | | | |
| | | | 0102; DQB1-0501_DQA1-0101; DRB3_0202; DRB1_0802; | | | |
| | | | DRB1_0701; DRB1_0401; DRB1_0407 | | | |
| 1 | NGGDA | N | DRB4_0103; DRB3_0101; DRB1_1104; DRB3_0301; | 0.825314 | 0.821672 | 0.411628 |
| | ALALL | | DRB1_0804; DRB1_1301; DRB1_1406; DRB1_1405; DQB1- | | | |
| | LLDRL | | 0303_DQA1-0301; DRB1_1102; DRB1_1101 | | | |
| | NQLES | | | | | |
| | KMSGK | | | | | |
| 2 | VTPSG | N | DRB1_0701; DRB5_0101; DRB1_0301; DRB1_1303; | 0.961338 | 0.963945 | 0.661484 |
| | TWLTY | | DRB1_1502; DRB4_0103; DRB3_0101; DRB1_1104; | | | |
| | TGAIK | | DRB3_0301; DRB1_0804; DRB1_1301; DRB1_1406; | | | |
| | LDDKD | | DRB1_1405; DQB1-0303_DQA1-0301; DRB1_1102; | | | |
| | PNFKD | | DRB1_1101 | | | |
| 3 | GLPYG | N | DRB1_1001; DQB1-0602_DQA1-0102; DRB1_0401; | 0.989536 | 0.990346 | 0.762635 |
| | ANKDG | | DRB1_1503; DRB1_0403; DRB1_0404; DRB1_0407; DQB1- | | | |
| | IIWVAT | | 0202_DQA1-0201; DPB1-0101_DPA1-0201; DRB4_0103; | | | |
| | EGALN | | DRB3_0101; DRB1_1104; DRB3_0301; DRB1_0804; | | | |
| | TPKD | | DRB1_1301; DRB1_1406; DRB1_1405; DQB1-0303_DQA1- | | | |
| | | | 0301; DRB1_1102; DRB1_1101; DRB1_0701; DRB5_0101; | | | |
| | | | DRB1_0301; DRB1_1303; DRB1_1502 | | | |
| 4 | RPQGL | IN | DQB1-0601_DQA1-0103; DRB1_1402; DQB1-0303_DQA1- | 0.993939 | 0.993903 | 0.872582 |
| | PNNTA | | 0201; DRB1_0802; DQB1-0302_DQA1-0301; DQB1- | | | |
| | SWFTA | | 0502_DQA1-0102; DRB4_0103; DRB3_0101; DRB1_1104; | | | |
| | LTQHG | | DRB3_0301; DRB1_0804; DRB1_1301; DRB1_1406; | | | |
| | KEDLK | | DRB1_1405; DQB1-0303_DQA1-0301; DRB1_1102; | | | |
| | | | DRB1_1101; DRB1_0701; DRB5_0101; DRB1_0301; | | | |
| | | | DRB1_1303; DRB1_1502; DRB1_1001; DQB1-0602_DQA1- | | | |
| | | | 0102; DRB1_0401; DRB1_1503; DRB1_0403; DRB1_0404; | | | |
| | | | DRB1_0407; DQB1-0202_DQA1-0201; DPB1-0101_DPA1- | | | |
| | | | 0201 | | | |
| 5 | GTTLP | N | DPB1-0601_DPA1-0103; DQB1-0401_DQA1-0301; | 0.996624 | 0.996645 | 0.914328 |
| | KGFYA | | DRB1_1601; DQB1-0201_DQA1-0501; DRB4_0103; | | | |
| | EGSRG | | DRB3_0101; DRB1_1104; DRB3_0301; DRB1_0804; | | | |
| | GSQAS | | DRB1_1301; DRB1_1406; DRB1_1405; DQB1-0303_DQA1- | | | |
| | SRSSS | | 0301; DRB1_1102; DRB1_1101; DRB1_0701; DRB5_0101; | | | |
| | | | DRB1_0301; DRB1_1303; DRB1_1502; DRB1_1001; DQB1- | | | |
| | | | 0602_DQA1-0102; DRB1_0401; DRB1_1503; DRB1_0403; | | | |
| | | | DRB1_0404; DRB1_0407; DQB1-0202_DQA1-0201; DPB1- | | | |
| | | | 0101_DPA1-0201; DQB1-0601_DQA1-0103; DRB1_1402; | | | |
| | | | DQB1-0303_DQA1-0201; DRB1_0802; DQB1-0302_DQA1- | | | |
| | | | 0301; DQB1-0502_DQA1-0102 | | | |
| 6 | IKLDD | N | DRB1_0803; DRB1_0801; DRB3_0202; DRB3_0201; | 0.999218 | 0.999236 | 0.930092 |
| | KDPNF | | DRB4_0103; DRB3_0101; DRB1_1104; DRB3_0301; | | | |
| | KDQVI | | DRB1_0804; DRB1_1301; DRB1_1406; DRB1_1405; DQB1- | | | |
| | LLNKH | | 0303_DQA1-0301; DRB1_1102; DRB1_1101; DRB1_0701; | | | |
| | IDAYK | | DRB5_0101; DRB1_0301; DRB1_1303; DRB1_1502; | | | |
| | | | DRB1_1001; DQB1-0602_DQA1-0102; DRB1_0401; | | | |
| | | | DRB1_1503; DRB1_0403; DRB1_0404; DRB1_0407; DQB1- | | | |
| | | | 0202_DQA1-0201; DPB1-0101_DPA1-0201; DQB1- | | | |
| | | | 0601_DQA1-0103; DRB1_1402; DQB1-0303_DQA1-0201; | | | |
| | | | DRB1_0802; DQB1-0302_DQA1-0301; DQB1-0502_DQA1- | | | |
| | | | 0102; DPB1-0601_DPA1-0103; DQB1-0401_DQA1-0301; | | | |
| | | | DRB1_1601; DQB1-0201_DQA1-0501 | | | |
| 7 | RQKKQ | N | DRB1_0102; DRB1_0101; DRB5_0202; DRB1_1202; | 0.999695 | 0.999724 | 0.960521 |
| | QTVTL | | DRB1_1201; DRB4_0103; DRB3_0101; DRB1_1104; | | | |
| | LPAAD | | DRB3_0301; DRB1_0804; DRB1_1301; DRB1_1406; | | | |
| | LDDFS | | DRB1_1405; DQB1-0303_DQA1-0301; DRB1_1102; | | | |
| | KQLQQ | | DRB1_1101; DRB1_0701; DRB5_0101; DRB1_0301; | | | |
| | | | DRB1_1303; DRB1_1502; DRB1_1001; DQB1-0602_DQA1- | | | |
| | | | 0102; DRB1_0401; DRB1_1503; DRB1_0403; DRB1_0404; | | | |
| | | | DRB1_0407; DQB1-0202_DQA1-0201; DPB1-0101_DPA1- | | | |
| | | | 0201; DQB1-0601_DQA1-0103; DRB1_1402; DQB1- | | | |
| | | | 0303_DQA1-0201; DRB1_0802; DQB1-0302_DQA1-0301; | | | |
| | | | DQB1-0502_DQA1-0102; DPB1-0601_DPA1-0103; DQB1- | | | |
| | | | 0401_DQA1-0301; DRB1_1601; DQB1-0201_DQA1-0501; | | | |
| | | | DRB1_0803; DRB1_0801; DRB3_0202; DRB3_0201 | | | |
| 8 | KPRQK | N | DPB1-0301_DPA1-0103; DRB5_0102; DPB1-0901_DPA1- | 0.999814 | 0.999831 | 0.968823 |
| | RTATK | | 0201; DRB4_0103; DRB3_0101; DRB1_1104; DRB3_0301; | | | |
| | AYNVT | | DRB1_0804; DRB1_1301; DRB1_1406; DRB1_1405; DQB1- | | | |
| | QAFGR | | 0303_DQA1-0301; DRB1_1102; DRB1_1101; DRB1_0701; | | | |
| | RGPEQ | | DRB5_0101; DRB1_0301; DRB1_1303; DRB1_1502; | | | |
| | | | DRB1_1001; DQB1-0602_DQA1-0102; DRB1_0401; | | | |
| | | | DRB1_1503; DRB1_0403; DRB1_0404; DRB1_0407; DQB1- | | | |
| | | | 0202_DQA1-0201; DPB1-0101_DPA1-0201; DQB1- | | | |
| | | | 0601_DQA1-0103; DRB1_1402; DQB1-0303_DQA1-0201; | | | |
| | | | DRB1_0802; DQB1-0302_DQA1-0301; DQB1-0502_DQA1- | | | |
| | | | 0102; DPB1-0601_DPA1-0103; DQB1-0401_DQA1-0301; | | | |
| | | | DRB1_1601; DQB1-0201_DQA1-0501; DRB1_0803; | | | |
| | | | DRB1_0801; DRB3_0202; DRB3_0201; DRB1_0102; | | | |
| | | | DRB1_0101; DRB5_0202; DRB1_1202; DRB1_1201 | | | |
| 9 | LPAAD | N | DRB1_0405; DRB1_0901; DRB1_0411; DRB1_1302; | 0.999905 | 0.999894 | 0.992082 |
| | LDDFS | | DRB4_0103; DRB3_0101; DRB1_1104; DRB3_0301; | | | |
| | KQLQQ | | DRB1_0804; DRB1_1301; DRB1_1406; DRB1_1405; DQB1- | | | |
| | SMSSA | | 0303_DQA1-0301; DRB1_1102; DRB1_1101; DRB1_0701; | | | |
| | DSTQA | | DRB5_0101; DRB1_0301; DRB1_1303; DRB1_1502; | | | |
| | | | DRB1_1001; DQB1-0602_DQA1-0102; DRB1_0401; | | | |
| | | | DRB1_1503; DRB1_0403; DRB1_0404; DRB1_0407; DQB1- | | | |
| | | | 0202_DQA1-0201; DPB1-0101_DPA1-0201; DQB1- | | | |
| | | | 0601_DQA1-0103; DRB1_1402; DQB1-0303_DQA1-0201; | | | |
| | | | DRB1_0802; DQB1-0302_DQA1-0301; DQB1-0502_DQA1- | | | |
| | | | 0102; DPB1-0601_DPA1-0103; DQB1-0401_DQA1-0301; | | | |
| | | | DRB1_1601; DQB1-0201_DQA1-0501; DRB1_0803; | | | |
| | | | DRB1_0801; DRB3_0202; DRB3_0201; DRB1_0102; | | | |
| | | | DRB1_0101; DRB5_0202; DRB1_1202; DRB1_1201; DPB1- | | | |
| | | | 0301_DPA1-0103; DRB5_0102; DPB1-0901_DPA1-0201 | | | |
| 10 | KDGII | N | DQB1-0603_DQA1-0103; DRB4_0103; DRB3_0101; | 0.999927 | 0.999918 | 0.992082 |
| | WVATE | | DRB1_1104; DRB3_0301; DRB1_0804; DRB1_1301; | | | |
| | GALNT | | DRB1_1406; DRB1_1405; DQB1-0303_DQA1-0301; | | | |
| | PKDHI | | DRB1_1102; DRB1_1101; DRB1_0701; DRB5_0101; | | | |
| | GTRNP | | DRB1_0301; DRB1_1303; DRB1_1502; DRB1_1001; DQB1- | | | |
| | | | 0602_DQA1-0102; DRB1_0401; DRB1_1503; DRB1_0403; | | | |
| | | | DRB1_0404; DRB1_0407; DQB1-0202_DQA1-0201; DPB1- | | | |
| | | | 0101_DPA1-0201; DQB1-0601_DQA1-0103; DRB1_1402; | | | |
| | | | DQB1-0303_DQA1-0201; DRB1_0802; DQB1-0302_DQA1- | | | |
| | | | 0301; DQB1-0502_DQA1-0102; DPB1-0601_DPA1-0103; | | | |
| | | | DQB1-0401_DQA1-0301; DRB1_1601; DQB1-0201_DQA1- | | | |
| | | | 0501; DRB1_0803; DRB1_0801; DRB3_0202; DRB3_0201; | | | |
| | | | DRB1_0102; DRB1_0101; DRB5_0202; DRB1_1202; | | | |
| | | | DRB1_1201; DPB1-0301_DPA1-0103; DRB5_0102; DPB1- | | | |
| | | | 0901_DPA1-0201; DRB1_0405; DRB1_0901; DRB1_0411; | | | |
| | | | DRB1_1302 | | | |

Index for Table: SI, Selection; P, viral protein; S, Surface glycoprotein; M, membrane glycoprotein; NP, nucleocapsid phosphoprotein.

### Leveraging Proteomic Data to Infer Relative Viral Protein Abundance

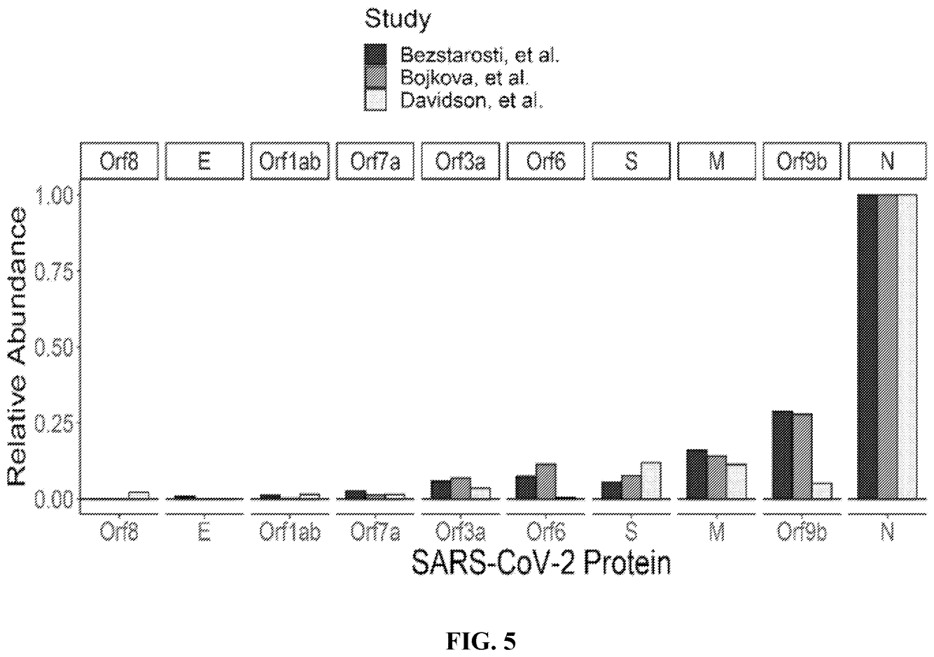

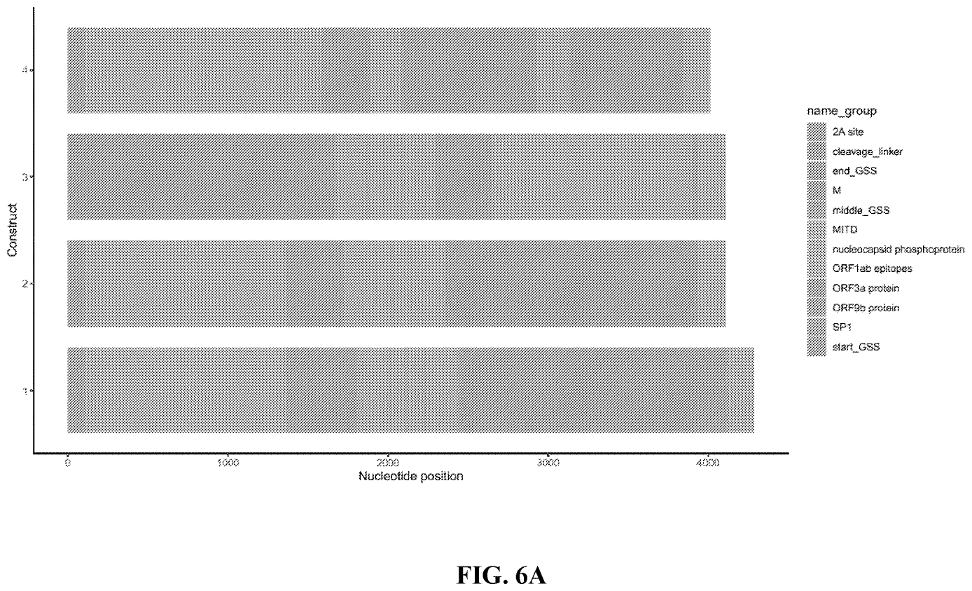

In addition to peptide-MHC binding, another important consideration in the design of a potential SARS CoV-2 vaccine is the degree of viral protein expression in infected host cells. In order to determine the relative abundance of SARS CoV-2 proteins, we analyzed three publicly available proteomic datasets that acquired unbiased LC-MS/MS on tryptic digestions of SARS-CoV-2-infected host cells. Relative abundance of the viral proteins was estimated by spectral counting, a semi-quantitative approach whereby peptide-spectrum matches are counted, and totals are compared across proteins (Table 8). Table 8 shows spectral counts from published SARS CoV-2 proteomic datasets. MS/MS spectra assigned to peptides from SARS CoV-2 proteins were tallied across datasets, divided by protein length, and normalized within each dataset to generate FIG. 5. This analysis demonstrated the significantly wide range of expression levels of the SARS CoV-2 proteins. Specifically, it confirmed that the N protein is the most abundant viral protein across all three datasets following SARS CoV-2 infection (FIG. 5). This finding is corroborated by reports of N-derived peptides being detected in gargle solution samples from COVID-19 patients. Furthermore, the N protein has been used as a biomarker for diagnosing patients infected with the SARS-CoV virus. On the other hand, based solely on genomic information, ORF10 might be considered a potential target for vaccine development. However, there is very little proteomic and transcriptomic evidence that ORF10 is actually expressed in SARS CoV-2 infected cells. These findings emphasize the value of considering SARS CoV-2 protein expression levels in addition to HLA binding predictions and the immunogenicity of these epitopes in vaccine design strategies.

TABLE 8

| | | | | | | | | PSMs/Length- | | |
|---|---|---|---|---|---|---|---|---|---|---|
| | Seq. | PSMs | | | PSMs/Length | | | relative (%) | | |
| Protein | length | Dav. | Boj. | Bez. | Dav. | Boj. | Bez. | Dav. | Boj. | Bez. |
| S | 1273 | 1778 | 112 | 109 | 1.396700707 | 0.087981147 | 0.085624509 | 12 | 7.68 | 5.57 |
| Pp1ab | 7096 | 1245 | 17 | 131 | 0.175450958 | 0.002395716 | 0.018461105 | 1.51 | 0.21 | 1.20 |
| Orf9b | 97 | 59 | 31 | 43 | 0.608247423 | 0.319587629 | 0.443298969 | 5.23 | 27.9 | 28.84 |
| Orf8 | 121 | 32 | | | 0.26446281 | 0 | 0 | 2.27 | 0.00 | 0.00 |
| Orf7a | 121 | 21 | 2 | 5 | 0.173553719 | 0.016528926 | 0.041322314 | 1.49 | 1.44 | 2.69 |
| Orf6 | 61 | 4 | 8 | 7 | 0.06557377 | 0.131147541 | 0.114754098 | 0.56 | 11.45 | 7.47 |
| Orf3a | 275 | 115 | 22 | 25 | 0.418181818 | 0.08 | 0.090909091 | 3.59 | 6.98 | 5.91 |
| N | 419 | 4876 | 480 | 644 | 11.6372315 | 1.145584726 | 1.53699284 | 100 | 100 | 100 |
| E | 75 | 1 | | 1 | 0.013333333 | 0 | 0.013333333 | 0.11 | 0.00 | 0.87 |
| M | 222 | 293 | 36 | 55 | 1.31981982 | 0.162162162 | 0.247747748 | 11.34 | 14.16 | 16.12 |

Table index:
Boj, Bojkova et al.: PXD017710;
Bez, Bezstarosti et al.: PXD018760;
Dav, Davidson et al.: PXD018241.

### DISCUSSION

In this work, the utility and validity of the HLA-I and HLA-II binding prediction algorithms used were demonstrated to the Coronaviridae virus family, and specifically to SARS-CoV-2. The strength of the prediction is two-fold: first, we have MS-based validated predictors for both HLA-I and HLA-II binders, which potentially could be leveraged to induce both long-term CD4+ and CD8+ T cell immunity against the virus. Specifically, our HLA-II predictor, which has also been trained on a large set of mono-allelic MS data, has been shown to significantly outperform previously published tools and is used here to identify high-quality CD4+epitopes that may contribute to both cellular and humoral immunity. Second, our expansive database of supported HLA-I and HLA-II alleles provides us with the ability to not only identify many peptide-MHC allele pairs, but to generate a narrow list of peptides with many potential HLA pairings that could be presented by the entire USA, European and Asian Pacific Islander populations. By applying these algorithms to previously assayed peptide-MHC allele pairs in ViPR, we were able to demonstrate excellent concordance between our binding predictions and the results of the binding assays for both HLA-I and HLA-II epitopes. We leveraged the homology within the Coronaviridae family to demonstrate that an exceedingly high portion (~90%) of our high-ranking SARS CoV-2 peptide-MHC allele pairs for which validation was available was indeed confirmed to bind the predicted MHC allele. We also confirmed that our binding predictors can identify epitopes that are immunogenic and can lead to CD8+ T cell responses to multiple SARS CoV-2 proteins in donor PBMCs. It is plausible that our significant fraction of experimentally confirmed epitopes (of all highly predicted, tested epitopes) is only an underestimate for overall immunogenicity, since PBMCs from only three donors were used in this initial experiment. Though we did not perform T cell assays to evaluate the immunogenicity of the HLA-II predicted epitopes, such analysis would be valuable, especially given the importance of CD4+ T cells in both the cellular and humoral anti-viral response. We thus propose that a combination of B and T cell epitopes could provide long-lasting immunity from SARS CoV-2 or mitigate the severity of disease when protection is partial.

We therefore concluded that using MS-based HLA binding predictors to predict T cell epitopes from the ORFs of SARS CoV-2 provides a significantly expanded, novel set of high-quality T cell vaccine targets for the virus. This was specifically the case when comparing this study to the recent publication by Grifoni, et al., We provide ten-fold more highly predicted epitopes, across many more HLA alleles which allow us to better prioritize vaccine candidates. In addition, we provide not only bioinformatic validation with a larger set of previously reported epitopes from other viruses from the Coronaviridae family in ViPR, but also experimentally validated, novel 2019 SARS CoV-2 T cell epitopes.

The selection of target sequences can be further guided by protein expression, epitopes predicted to provide coverage to a big fraction of the population, and conserved 2019 SARS CoV-2 epitopes. First, designing therapeutics against predicted epitopes is only effective if the proteins containing those epitopes are expressed at high enough levels for efficient antigen processing and presentation to take place. Therefore, it is crucial that protein expression be considered when selecting therapeutic targets. Second, prioritization of epitopes that are predicted to bind multiple alleles could provide coverage to significant fractions of the population, while including few epitopes in the vaccine. Lastly, during the viral spread and expansion through the population, genomic modifications are acquired, generating sequence diversity among the 2019 SARS CoV-2 population. This diversity may allow evasion of immune pressure, and therefore it is important to prioritize epitopes that are conserved across the 2019 SARS CoV-2 population.

While limiting epitope selection to highly expressed proteins, epitopes predicted to bind multiple high frequency HLA alleles, and conserved viral epitopes restricts the number of potential epitopes, the breadth of the list we provide increases the likelihood of identifying many high-quality, highly expressed epitopes. The epitopes characterized here, combined with insights on 2019 SARS CoV-2 protein expression along with further efforts to confirm immunogenicity, can provide pre-clinical validation of epitopes that may be vaccine candidates to induce strong cellular immunity.

### CONCLUSIONS

In summary, the work provides the most extensive set of both CD4+ and CD8+ T cells epitopes that are spanning the entire 2019 SARS CoV-2 genome and binding a wide set of HLA-I and HLA-II alleles. Combining this epitope list to protein expression levels, population coverage and viral sequence conservation will lead to generation of a short list of vaccine epitope candidates that are likely immunogenic in the majority of the population. Our predicted list of CD4+ and CD8+ T cell epitopes will complement B cell epitopes and serve as a resource for the scientific community to generate potent 2019 SARS CoV-2 vaccine epitopes and generate long-lasting T cell immunity.

### Example 2: HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing. 2019 SARS CoV-2 infected cell lines were prepared. Cell lysates are prepared and HLA molecules purified in accordance with disclosed protocols (Sidney et al., Current Protocols in Immunology 18.3.1 (1998); Sidney, et al., J. Immunol. 154:247 (1995); Sette, et al., Mol. Immunol. 31:813 (1994)). HLA molecules are purified from lysates by affinity chromatography. The lysates are passed over a column of Sepharose CL-4B beads coupled to an appropriate antibody. The anti-HLA column is then washed with 10 mM Tris-HCL, pH 8.0, in 1% NP-40, PBS, and PBS containing 0.4% n-octylglucoside and HLA molecules are eluted with 50 mM diethylamine in 0.15M NaCl containing 0.4% n-octylglucoside, pH 11.5. A 1/25 volume of 2.0M Tris, pH 6.8, is added to the eluate to reduce the pH. Eluates are then concentrated by centrifugation in Centriprep 30 concentrators (Amicon, Beverly, MA). Protein content is evaluated by a BCA protein assay (Pierce Chemical Co., Rockford, IL) and confirmed by SDS-PAGE.

A detailed description of the protocol utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) are incubated with various unlabeled peptide inhibitors and 1-10 nM ¹²⁵I-radiolabeled probe peptides for 48 h in PBS containing 0.05% Nonidet P40 (NP40) (or 20% w/v digitonin for H-2 IA assays) in the presence of a protease inhibitor cocktail. All assays are at pH 7.0 with the exception of DRB1*0301, which was performed at pH 4.5, and DRB1*1601 (DR2w21131) and DRB4*0101 (DRw53), which were performed at pH 5.0.

Following incubation, MHC-peptide complexes are separated from free peptide by gel filtration on 7.8 mm×15 cm TSK200 columns (TosoHaas 16215, Montgomeryville, PA). Because the large size of the radiolabeled peptide used for the DRB1*1501 (DR2w2 (31) assay makes separation of bound from unbound peaks more difficult under these conditions, all DRB1*1501 (DR2w2 (31) assays were performed using a 7.8 mm×30 cm TSK2000 column eluted at 0.6 mLs/min. The eluate from the TSK columns is passed through a Beckman 170 radioisotope detector, and radioactivity is plotted and integrated using a Hewlett-Packard 3396A integrator, and the fraction of peptide bound is determined.

Radiolabeled peptides are iodinated using the chloramine-T method. Typically, in preliminary experiments, each MHC preparation is titered in the presence of fixed amounts of radiolabeled peptides to determine the concentration of HLA molecules necessary to bind 10-20% of the total radioactivity. All subsequent inhibition and direct binding assays are performed using these HLA concentrations.

Since under these conditions [label]< [HLA] and IC50> [HLA], the measured IC50 values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC50 of a positive control for inhibition by the IC50 for each tested peptide (typically unlabeled versions of the radiolabeled probe peptide). For database purposes, and inter-experiment comparisons, relative binding values are compiled. These values can subsequently be converted back into IC50 nM values by dividing the IC50 nM of the positive controls for inhibition by the relative binding of the peptide of interest. This method of data compilation has proven to be the most accurate and consistent for comparing peptides that have been tested on different days, or with different lots of purified MHC.

Because the antibody used for HLA-DR purification (LB3.1) is a-chain specific, 131 molecules are not separated from 133 (and/or 134 and (35) molecules. The 131 specificity of the binding assay is obvious in the cases of DRB1*0101 (DR1), DRB1*0802 (DR8w2), and DRB1*0803 (DR8w3), where no 133 is expressed. It has also been demonstrated for DRB1*0301 (DR3) and DRB3*0101 (DR52a), DRB1*0401 (DR4w4), DRB1*0404 (DR4w14), DRB1*0405 (DR4w15), DRB1*1101 (DR5), DRB1*1201 (DR5w12), DRB1*1302 (DR6w19) and DRB1*0701 (DR7). The problem of13 chain specificity for DRB1*1501 (DR2w2 (31), DRB5*0101 (DR2w2 (32), DRB1*1601 (DR2w21131), DRB5*0201 (DR51Dw21), and DRB4*0101 (DRw53) assays is circumvented by the use of fibroblasts. Development and validation of assays with regard to DRP molecule specificity have been described previously (see, e.g., Southwood et al., J. Immunol. 160:3363-3373, 1998).

The live cell/flow cytometry-based assays can also be used. This is a well-established assay utilizing the TAP-deficient hybridoma cell line T2 (American Type Culture Collection (ATCC Accession No. CRL-1992), Manassas, Va.). The TAP deficiency in this cell line leads to inefficient loading of MHCI in the ER and an excess of empty MHCIs. Salter and Cresswell, EMBO J. 5:94349 (1986); Salter, Immunogenetics 21:235-46 (1985). Empty MHCIs are highly unstable, and are therefore short-lived. When T2 cells are cultured at reduced temperatures, empty MHCIs appear transiently on the cell surface, where they can be stabilized by the exogenous addition of MHCI-binding peptides. To perform this binding assay, peptide-receptive MHCIs were induced by culturing aliquots of 107 T2 cells overnight at 26° C. in serum free AIM-V medium alone, or in medium containing escalating concentrations (0.1 to 100 μM) of peptide. Cells were then washed twice with PBS, and subsequently incubated with a fluorescent tagged HLA-A02:01-specific monoclonal antibody, BB7.2, to quantify cell surface expression. Samples were acquired on a FACS Calibur instrument (Becton Dickinson) and the mean fluorescence intensity (MFI) determined using the accompanying Cellquest software.

### Example 3: Confirmation of Immunogenicity

In an exemplary method for confirmation of immunogenicity, in vitro education (IVE) assays are used to test the ability of each test peptide to expand CD8+ T cells. Mature professional APCs are prepared for these assays in the following way. 80-90×10⁶ PBMCs from a healthy human donor are plated in 20 ml of RPMI media containing 2% human AB serum, and incubated at 37° C. for 2 hours to allow for plastic adherence by monocytes. Non-adherent cells are removed and the adherent cells are cultured in RPMI, 2% human AB serum, 800 IU/ml of GM-CSF and 500 IU/ml of IL-4. After 6 days, TNF-alpha is added to a final concentration of 10 ng/ml. On day 7, the dendritic cells (DC) are matured either by the addition of 12.5 mg/ml poly I: C or 0.3 μg/ml of CD40L. The mature dendritic cells (mDC) are harvested on day 8, washed, and either used directly or cryopreserved for future use.

For the IVE of CD8+ T cells, aliquots of 2×10⁵ mDCs are pulsed with each peptide at a final concentration of 100 micromole, incubated for 4 hours at 37° C., and then irradiated (2500 rads). The peptide-pulsed mDCs are washed twice in RPMI containing 2% human AB serum. 2×10⁵ mDCs and 2×10⁶ autologous CD8+ cells are plated per well of a 24-well plate in 2 ml of RPMI containing 2% human AB, 20 ng/ml IL-7 and 100 μg/ml of IL-12, and incubated for 12 days. The CD8+ T cells are then re-stimulated with peptide-pulsed, irradiated mDCs. Two to three days later, 20 IU/ml IL-2 and 20 ng/IL7 are added. Expanding CD8+ T cells are re-stimulated every 8-10 days, and are maintained in media containing IL-2 and IL-7. Cultures are monitored for peptide-specific T cells using a combination of functional assays and/or tetramer staining. Parallel IVES with the modified and parent peptides allowed for comparisons of the relative efficiency with which the peptides expanded peptide-specific T cells.

### Quantitative and Functional Assessment of CD8+ and CD4+ T Cells

### Tetramer Staining

MHC tetramers are purchased or manufactured on-site, and are used to measure peptide-specific T cell expansion in the IVE assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8+/Tetramer+.

CD4+ T cell responses towards the peptide antigens can be tested using the ex vivo induction protocol. In this example, CD4+ T cell responses were identified by monitoring IFNγ and/or TNFα production in an antigen specific manner.

Evaluation of Antigen Presentation: For a subset of predicted antigens, the affinity of the viral epitopes for the indicated HLA alleles and stability of the neoepitopes with the HLA alleles was determined. An exemplary detailed description of the protocol utilized to measure the binding affinity of peptides to Class I MHC has been published (Sette et al, Mol. Immunol. 31 (11): 813-22, 1994). In brief, MHCI complexes were prepared and bound to radiolabeled reference peptides. Peptides were incubated at varying concentrations with these complexes for 2 days, and the amount of remaining radiolabeled peptide bound to MHCI was measured using size exclusion gel-filtration. The lower the concentration of test peptide needed to displace the reference radiolabeled peptide demonstrates a stronger affinity of the test peptide for MHCI. Peptides with affinities to MHCI<50 nM are generally considered strong binders while those with affinities <150 nM are considered intermediate binders and those <500 nM are considered weak binders (Fritsch et al, 2014).

An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (B2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts. MHC-II binding affinity with peptides is measured following the same general procedure as with measuring MHCI-peptide binding affinity. Prediction algorithms utilized for predicting MHCII alleles for binding to a given peptide are described herein. Besides, NetMHCIIpan may be utilized for prediction of binding.

To assess whether antigens could be processed and presented from the larger polypeptide context, peptides eluted from HLA (class I or class II) molecules isolated from cells expressing the genes of interest were analyzed by tandem mass spectrometry (MS/MS).

### ELISPOT

Peptide-specific T cells are functionally enumerated using the ELISPOT assay (BD Biosciences), which measures the release of IFNgamma from T cells on a single cell basis. Target cells (T2 or HLA-A0201 transfected C1Rs) were pulsed with 10 μM peptide for 1 hour at 37° C., and washed three times. 1×10⁵ peptide-pulsed targets are co-cultured in the ELISPOT plate wells with varying concentrations of T cells (5×10² to 2×10³) taken from the IVE culture. Plates are developed according to the manufacturer's protocol, and analyzed on an ELISPOT reader (Cellular Technology Ltd.) with accompanying software. Spots corresponding to the number of IFNgamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

### CD107 Staining

CD107a and b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107 a and b staining is reported on a single cell basis, the assay is used to functionally enumerate peptide-specific T cells. To perform the assay, peptide is added to HLA-A0201-transfected cells C1R to a final concentration of 20 μM, the cells were incubated for 1 hour at 37° C., and washed three times. 1×10⁵ of the peptide-pulsed CIR cells were aliquoted into tubes, and antibodies specific for CD107 a and b are added to a final concentration suggested by the manufacturer (Becton Dickinson). Antibodies are added prior to the addition of T cells in order to “capture” the CD107 molecules as they transiently appear on the surface during the course of the assay. 1×10⁵ T cells from the culture are added next, and the samples were incubated for 4 hours at 37° C. The T cells are further stained for additional cell surface molecules such as CD8 and acquired on a FACS Calibur instrument (Becton Dickinson). Data is analyzed using the accompanying Cellquest software, and results were reported as the percentage of CD8+CD107 a and b+ cells.

### CTL Lysis

Cytotoxic activity is measured using a chromium release assay. Target T2 cells are labeled for 1 hour at 37° C. with Na⁵¹Cr and washed 5×10³ target T2 cells were then added to varying numbers of T cells from the IVE culture. Chromium release is measured in supernatant harvested after 4 hours of incubation at 37° C. The percentage of specific lysis is calculated as:

Experimental release−spontaneous release/Total release−spontaneous release×100

### Example 4: Peptide Composition for Prophylactic or Therapeutic Uses

Immunogenic or vaccine compositions of the present disclosure are used to inhibit viral replication. For example, a polyepitopic composition (or a nucleic acid comprising the same) containing multiple CTL and HTL epitopes is administered to individuals having viral infections. The composition is provided as a single lipidated polypeptide that encompasses multiple epitopes. The composition is administered in an aqueous carrier comprised of alum. The dose of peptide for the initial immunization is from about 1 to about 50,000 lig, generally 100-5,000 ps, for a 70 kg patient. The initial administration is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious to inhibit viral replication.

Alternatively, the polyepitopic composition can be administered as a nucleic acid, for example as RNA, in accordance with methodologies known in the art and disclosed herein.

Viral epitope binding agents, such as TCR or CARs can be administered in accordance with methodologies known in the art and disclosed herein. The binding agents can be administered as polypeptides or polynucleotides, for example RNA, encoding the binding agents, or as a cellular therapy, by administering cells expressing the binding agents.

Viral epitope peptides, polynucleotides, binding agents, or cells expressing these molecules can be delivered to the same patient via multiple methodologies known in the art, and can further be combined with other therapies (e.g., anti-viral therapies).

### Example 5. Administration of Compositions Using Dendritic Cells

Vaccines comprising epitopes of the present disclosure may be administered using dendritic cells. In this example, the peptide-pulsed dendritic cells can be administered to a patient to stimulate a CTL response in vivo. In this method dendritic cells are isolated, expanded, and pulsed with a vaccine comprising peptide CTL and HTL epitopes of the present disclosure. The dendritic cells are infused back into the patient to elicit CTL and HTL responses in vivo. The induced CTL and HTL then destroy (CTL) or facilitate destruction (HTL) of the specific target cells that bear the proteins from which the epitopes in the vaccine are derived.

Alternatively, ex vivo CTL or HTL responses to a particular viral antigen can be induced by incubating in tissue culture the patient's, or genetically compatible, CTL or HTL precursor cells together with a source of antigen-presenting cells, such as dendritic cells, and the appropriate immunogenic peptides.

After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., cells displaying viral epitopes.

### Example 6: Identification of Mutant Sequences with Immunogenic Potential

For each epitope, the full-length amino acid sequence of the viral epitope was derived. Any constituent 9-mer or 10-mer protein sequence was scored for binding potential on six common HLA alleles (HLA-A01: 01, HLA-A02:01. HLA-A03: 01, HLA-A24: 02, HLA-B07:02, and HLA-B08:01) using available algorithms. Any peptide scoring better than 1000 nM was nominated.

For each epitope, the full-length amino acid sequence of the viral epitope was derived. Any constituent 9mer or 10mer not found in the germline protein sequence was flagged and scored for binding potential on six common HLA alleles (HLA-A01:01, HLA-A02:01. HLA-A03:01, HLA-A24:02, HLA-B07:02, and HLA-B08:01) using available algorithms.

### Example 7: SARS CoV-2 (2019 SARS-Cov 2) Peptide String Designs

Provided herein are special constructs “strings” of multiple 2019 SARS CoV-2 nucleocapsid epitopes for therapeutic application. These strings are designed to contain specific epitopes of the 2019 SARS CoV-2 nucleocapsid each of which are individually disclosed in the previous examples in this application, and predicted by the MHC-binding algorithm as described above. These strings are designed for therapeutic use in treating COVID 19 and can be administered as the nucleic acid string constructs, e.g. mRNA encapsulated in a lipid nanoparticle.

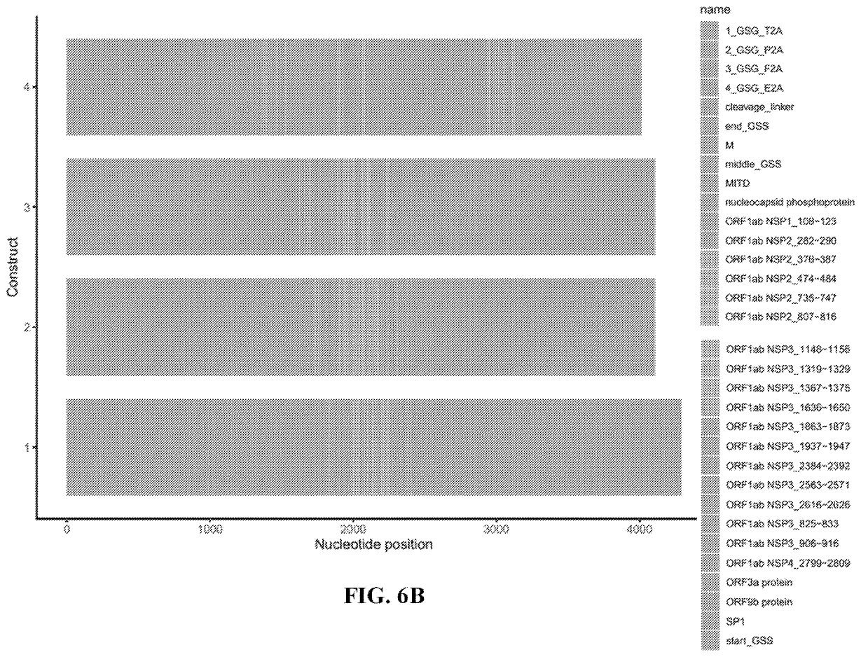

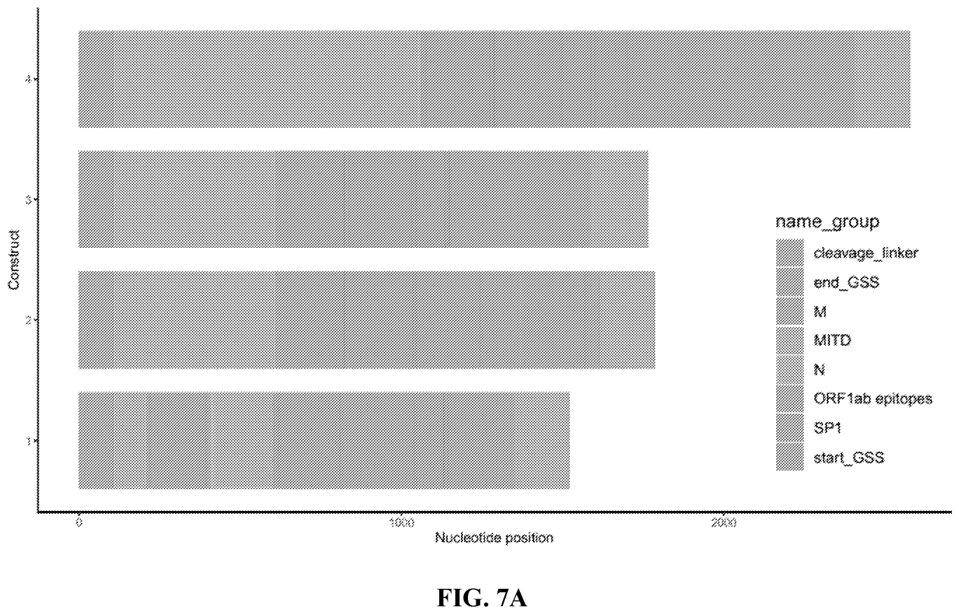

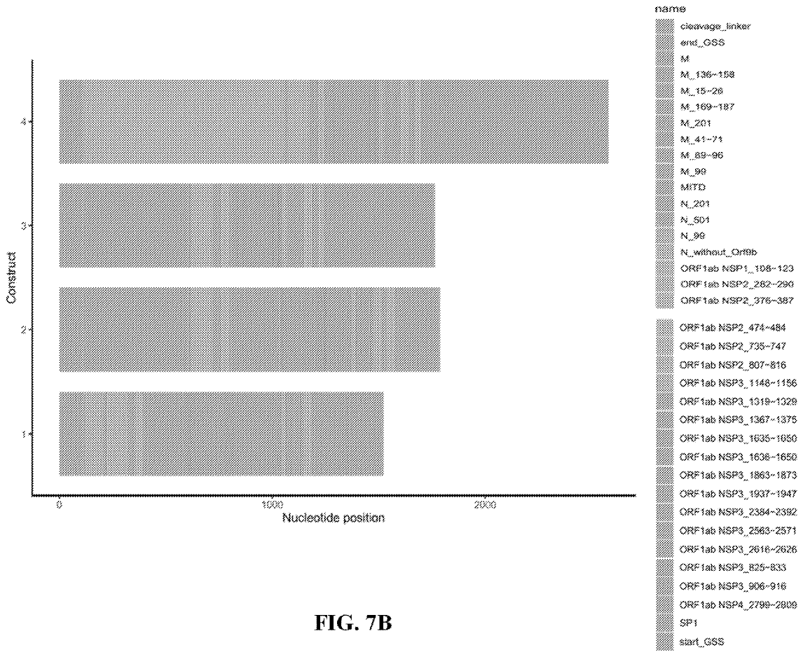

The strings are designed to include a 5′UTR and a 3′UTR. Epitopes are interconnected by peptide linkers, encoded by the respective nucleic acid sequences. Some linkers have specific cleavage sites. Table 11 and Table 12 show the complete sequences of amino acids and the nucleotide sequences encoding them. The nucleotide sequences are further codon optimized for efficient translation in human. Tables 9 and 10 provide construct maps, detailing the segments and sequences corresponding to each string in the Table 11 and 12 respectively. FIG. 6A and FIG. 6B exemplify graphically the design of the strings of Group 1, sequences of RS C1-C4. FIG. 6B display a more detailed layout of FIG. 6A. FIG. 7A and FIG. 7B exemplify graphically the design of the strings of Group 2, sequences of RS C5-C8. FIG. 7B displays a more detailed layout of FIG. 7A. In some cases, the strings are also identified as RS C1n, RS-C2n etc.

The string named “RS_C1 GSS linkers” (abbreviated RS C1) encodes an ORF having an amino acid length of 1266 amino acids, and the nucleotide sequence encoding the ORF is 3798 nucleotides long. The entire string is 4107 nucleotides long and encodes a peptide string that is 1369 amino acids long. Table 11 exemplifies the amino acid and nucleic acid sequences (not codon optimized). An exemplary codon optimized sequence for string named “RS C1 GSS linkers” is:

(SEQ ID RS C1n)
ATGAGGGTAATGGCTCCGCGCACCCTTATATTGCTTCTGTCTGGGGCGCTTGCGCTT
ACGGAAACTTGGGCAGGGTCTGGTGGGTCTGGAGGTGGTGGTTCCGGCGGGAGTGATAATG
GACCTCAGAACCAACGCAACGCACCCAGGATCACATTTGGTGGGCCATCCGACTCCACTGG
CAGCAACCAAAACGGTGAACGAAGTGGCGCGAGATCCAAGCAGCGCCGCCCTCAAGGTTT
GCCGAATAACACAGCCAGCTGGTTCACTGCCTTGACTCAGCATGGCAAGGAAGATTTGAAA
TTCCCACGAGGACAGGGTGTCCCTATAAATACGAATTCCAGTCCCGATGATCAGATCGGTTA
TTATAGAAGAGCTACAAGGCGAATCCGGGGGGGGGATGGCAAGATGAAAGACCTGAGCCC
GCGCTGGTATTTCTATTACCTGGGAACTGGACCTGAAGCGGGCCTCCCTTATGGTGCGAATA
AAGACGGAATAATCTGGGTGGCTACGGAAGGGGCCCTGAACACGCCCAAGGATCACATCG
GCACACGCAATCCGGCGAACAATGCCGCGATAGTGCTGCAGCTGCCACAAGGCACCACACT
GCCAAAGGGGTTTTACGCAGAAGGCTCCAGAGGTGGGTCACAAGCCTCTTCTCGATCCTCTT
CCCGGAGCAGAAATAGCTCACGAAACTCCACCCCGGGCAGTTCCAGAGGCACAAGTCCTGC
TCGCATGGCAGGTAATGGAGGTGACGCCGCTCTCGCGCTTCTTCTCCTCGACAGACTGAATC
AGCTTGAGAGTAAAATGAGTGGAAAGGGACAGCAGCAACAGGGGCAAACAGTGACCAAAA
AATCAGCTGCGGAAGCCAGCAAGAAGCCGCGCCAGAAACGGACAGCGACTAAAGCCTACA
ATGTTACCCAAGCCTTCGGCCGCAGAGGGCCGGAGCAAACTCAGGGCAACTTCGGCGATCA
GGAACTGATCCGCCAGGGAACAGATTATAAACATTGGCCCCAAATCGCACAATTTGCACCC
TCCGCGTCTGCGTTCTTCGGCATGAGCCGGATTGGTATGGAAGTAACACCGAGCGGCACCT
GGCTTACATATACAGGCGCGATTAAATTGGATGACAAGGATCCCAATTTTAAGGACCAAGT
GATATTGCTCAACAAACATATTGATGCGTATAAGACTTTTCCTCCTACTGAACCAAAGAAGG
ATAAGAAAAAAAAGGCTGATGAAACACAAGCTCTTCCTCAACGCCAGAAAAAGCAACAGA
CAGTTACCTTGCTCCCGGCGGCCGATCTTGATGATTTTTCCAAGCAGCTGCAACAGTCTATG
TCATCAGCCGACTCTACCCAAGCAGGCGGTTCAGGTGGCGGCGGTTCTGGTGGCGACCCTA
AGATATCCGAAATGCACCCCGCACTCAGACTGGTAGACCCCCAAATACAACTGGCGGTTAC
ACGGATGGAGAACGCGGTTGGCAGGGACCAGAATAACGTGGGGCCAAAGGTGTATCCTAT
CATCCTCAGATTGGGTAGTCCCCTCAGCTTGAATATGGCTAGAAAAACACTGAATTCATTGG
AAGACAAGGCGTTCCAACTGACACCGATTGCGGTGCAGATGACAAAGCTCGCTACAACCGA
GGAACTCCCAGACGAGTTTGTAGTAGTCACAGTCAAGGGTGGCTCAGGCGGCGGAGGCTCA
GGTGGATACCATTTTTTTCATACGACGGATCCATCTTTTCTCGGCCGATATATGAGCGCGCT
CTTCGCAGACGATCTGAATCAGCTCACGGGATACCACACAGACTTCAGTAGTGAAATTATC
GGTTATCAGTTGATGTGCCAGCCGATATTGTTGGCTGAGGCTGAACTTGCTAAGAATGTCTC
CCTCATCTTGGGGACAGTCAGCTGGAACCTTAAACGCCGCTACTTGCTGTCTGCGGGTATCT
TTGGGGCTATTACGGATGTATTTTACAAAGAAAACAGCTATAAAGTACCGACCGACAATTA
CATCACGACTTATGCACGCATGGCGGCACCAAAGGAAATCATATTTCTTGAGGGGGAAACT
CTGTTCGGAGACGATACAGTAATAGAAGTCGCTATTATACTTGCTTCATTTTCAGCCAGTAC
TCGACGCATGGCTATGGTGACCAATAATACTTTCACGCTGAAGGTTCCTCATGTGGGCGAAA
TCCCCGTCGCCTATCGCAAGGTCCTGCTCAAGACTATTCAACCTCGCGTTGAAAAGTACCTT
TTCGATGAAAGCGGGGAATTTAAACTCAGCGAAGTGGGCCCTGAACACTCACTCGCAGAAT
ATTATATTTTCTTTGCCTCCTTTTATTATAAACGGAATGGCGGCGGCTCTGGCGGAGGTGGG
TCTGGTGGCGATCTCTTTATGCGGATCTTTACAATAGGGACCGTTACATTGAAGCAAGGGGA
AATCAAGGACGCCACACCGTCCGATTTCGTTAGAGCAACCGCCACGATTCCTATCCAGGCA
TCCTTGCCCTTCGGGTGGCTGATAGTAGGTGTAGCACTCCTTGCAGTCTTTCAAAGCGCATC
CAAAATCATTACCCTCAAGAAACGCTGGCAGCTTGCCCTTTCTAAGGGAGTACATTTCGTAT
GTAATCTGTTGCTCCTGTTCGTTACAGTTTATAGCCATCTCTTGCTCGTTGCCGCTGGGCTGG
AAGCCCCATTTTTGTACCTGTACGCCCTTGTGTATTTTCTTCAAAGCATAAATTTCGTGAGGA
TTATCATGCGCCTCTGGCTGTGCTGGAAATGCCGCTCAAAAAATCCACTTCTTTATGACGCA
AACTATTTTCTCTGTTGGCATACAAATTGTTACGATTATTGTATACCTTACAACAGTGTGACG
TCCTCCATAGTCATCACCAGCGGAGATGGTACAACGTCACCCATTTCTGAGCACGACTACCA
AATAGGCGGCTATACGGAGAAGTGGGAATCTGGTGTAAAAGATTGCGTGGTGCTTCACTCT
TATTTTACTTCAGATTACTACCAGCTTTATAGCACTCAACTTTCTACCGATACAGGAGTGGA
ACACGTCACATTTTTTATATACAACAAGATTGTCGATGAACCCGAAGAACACGTGCAAATA
CATACAATCGACGGCTCCTCAGGAGTCGTCAATCCAGTCATGGAACCAATCTACGATGAGC
CGACAACTACCACTAGTGTACCGCTCGGGGGAAGCGGGGGCGGAGGTAGCGGCGGAGCAG
ACAGTAATGGTACTATAACTGTGGAGGAGCTCAAGAAGCTCCTTGAGCAATGGAATCTGGT
CATAGGTTTTCTGTTTCTTACCTGGATATGCCTTCTTCAGTTCGCCTATGCGAATCGCAACCG
CTTCCTGTACATCATAAAGCTCATATTCCTCTGGCTGCTCTGGCCGGTTACTCTTGCCTGTTT
TGTTCTTGCTGCTGTATACCGCATTAATTGGATAACGGGGGGAATAGCGATCGCGATGGCAT
GCTTGGTGGGATTGATGTGGCTGAGCTACTTTATAGCGTCATTTAGGCTTTTTGCGAGGACT
AGATCCATGTGGTCCTTTAATCCCGAAACTAACATTCTTCTCAATGTACCGTTGCATGGAAC
TATTTTGACTAGACCCCTTCTCGAGAGTGAGCTGGTGATAGGAGCCGTGATACTCAGGGGTC
ATCTCCGGATTGCCGGTCACCATTTGGGTAGATGTGACATAAAAGATCTCCCAAAGGAAAT
TACGGTAGCTACGTCTCGAACCCTTTCATACTACAAACTCGGTGCTAGCCAGCGAGTGGCTG
GGGATAGCGGCTTCGCGGCGTATTCTCGCTACAGAATTGGAAACTACAAGTTGAATACGGA
CCACTCATCAAGTAGCGATAACATTGCACTGCTTGTGCAGGGTGGTAGTCTCGGGGGGGGC
GGATCCGGTATCGTGGGCATAGTTGCGGGTCTCGCTGTGCTGGCTGTGGTCGTGATCGGCGC
GGTCGTAGCTACCGTGATGTGTAGGCGGAAAAGCAGTGGTGGTAAAGGTGGATCATATAGT
CAGGCTGCATCATCTGATTCCGCTCAAGGAAGCGACGTCAGCCTGACAGCTTGATAA

The string named “RS C2 GSS linkers inverted” (abbreviated RS C2) encodes an ORF having an amino acid length of 1266 amino acids, and the nucleotide sequence encoding the ORF is 3798 nucleotides long. The entire string is 4107 nucleotides long and encodes a peptide string that is 1369 amino acids long. Table 11 exemplifies the amino acid sequence and the nucleic acid sequences (not codon optimized) encoding the same. An exemplary codon optimized sequence for string named “RS C2 GSS linkers inverted” is:

(SEQ ID RS C2n)
ATGCGAGTCATGGCGCCGCGCACCCTGATACTTCTGCTTAGCGGCGCTTTGGCCCT
CACCGAGACATGGGCTGGCAGCGGTGGTTCTGGAGGCGGAGGATCAGGCGGAGCGGACAG
CAATGGCACGATCACTGTGGAGGAGCTCAAAAAACTTTTGGAACAATGGAATTTGGTAATT
GGTTTCTTGTTTCTTACTTGGATATGCCTGCTTCAGTTCGCCTATGCGAACAGAAATAGATTT
TTGTATATCATTAAATTGATATTTCTTTGGTTGCTTTGGCCTGTTACTCTGGCTTGTTTCGTCC
TCGCTGCGGTTTATCGGATAAATTGGATTACGGGTGGAATCGCAATTGCCATGGCCTGTCTG
GTCGGTCTGATGTGGCTTTCCTACTTCATAGCATCATTTAGGCTGTTCGCGAGAACGCGAAG
CATGTGGAGTTTCAACCCCGAGACGAATATCCTTTTGAACGTGCCTCTTCATGGCACTATTC
TCACTCGACCTCTGCTTGAATCCGAGCTCGTCATCGGCGCGGTAATCCTCCGGGGTCATCTG
CGGATCGCAGGTCACCACCTCGGGCGGTGTGATATCAAGGATCTTCCGAAAGAAATTACCG
TAGCTACTTCACGCACACTCAGCTACTACAAGCTGGGTGCTTCACAAAGAGTCGCCGGTGAT
TCTGGTTTCGCTGCGTATAGCAGGTACCGAATAGGAAATTACAAGCTCAATACCGATCATTC
CTCCAGCTCAGATAACATAGCCCTGCTTGTGCAAGGGGGATCCGGAGGAGGAGGTTCAGGC
GGTGACTTGTTTATGAGGATCTTTACCATCGGAACAGTGACACTCAAACAAGGGGAAATAA
AGGACGCCACTCCGTCAGACTTTGTTAGAGCAACAGCGACTATTCCGATTCAAGCCAGCCTT
CCTTTCGGGTGGCTCATAGTGGGCGTCGCATTGCTGGCGGTGTTTCAGAGTGCGAGTAAGAT
CATAACCCTCAAAAAGCGGTGGCAGTTGGCGTTGTCTAAAGGGGTACATTTTGTCTGCAACC
TTCTGCTCCTGTTCGTAACAGTTTATTCTCACCTGCTGTTGGTTGCGGCCGGTCTGGAGGCCC
CATTTCTTTATCTTTACGCACTTGTTTATTTCCTTCAATCCATAAATTTCGTTCGGATCATCAT
GAGATTGTGGCTCTGCTGGAAGTGCAGATCCAAAAACCCTCTCCTCTACGACGCGAATTATT
TCTTGTGTTGGCACACAAATTGCTATGACTATTGCATACCGTATAACTCCGTCACTTCTTCAA
TCGTAATCACCTCAGGCGACGGAACCACATCTCCCATCTCTGAGCACGACTACCAGATTGGC
GGATATACTGAAAAGTGGGAATCCGGTGTAAAGGACTGCGTAGTACTCCACTCATACTTCA
CTAGTGATTACTATCAACTCTACAGCACCCAGTTGAGCACTGATACGGGGGTCGAGCATGT
AACCTTCTTCATCTATAACAAAATAGTTGACGAGCCAGAGGAGCATGTACAAATACATACC
ATTGACGGTTCTTCTGGAGTCGTGAATCCGGTAATGGAACCTATTTATGATGAACCCACAAC
TACTACAAGTGTACCCCTTGGAGGCAGCGGCGGGGGTGGGTCTGGCGGATATCATTTCTTTC
ACACGACGGACCCTAGTTTTCTTGGTAGGTATATGAGCGCTCTTTTTGCGGATGATCTCAAT
CAGCTTACGGGCTACCACACGGACTTCAGTAGTGAAATAATCGGGTATCAATTGATGTGCC
AACCTATTCTGCTCGCGGAGGCAGAACTCGCCAAGAACGTTTCTCTGATCCTCGGCACGGTA
TCTTGGAATCTTAAAAGGAGATACCTTCTGAGCGCAGGCATTTTTGGCGCAATAACAGATGT
GTTTTACAAAGAAAATAGCTATAAGGTTCCTACAGACAACTACATAACCACATATGCAAGG
ATGGCAGCCCCGAAAGAAATTATATTCTTGGAGGGGGAGACTTTGTTCGGTGACGACACAG
TCATAGAGGTAGCAATTATACTCGCGAGCTTCTCCGCGTCTACTAGACGAATGGCGATGGTT
ACCAACAACACGTTTACGTTGAAGGTCCCCCACGTTGGCGAAATACCCGTCGCTTACAGAA
AGGTACTTCTCAAGACGATACAACCACGGGTGGAGAAGTATCTCTTCGACGAAAGTGGGGA
GTTTAAGCTTTCAGAAGTTGGGCCGGAACACTCCTTGGCGGAATACTATATTTTTTTTGCGT
CATTTTATTACAAGAGGAATGGGGGGGGTTCTGGGGGGGGTGGATCTGGCGGGGATCCTAA
GATCTCTGAGATGCACCCTGCCCTGCGCCTTGTGGATCCACAGATACAGTTGGCTGTCACGA
GAATGGAGAATGCGGTGGGCAGGGATCAGAATAACGTTGGTCCAAAGGTATACCCGATCAT
TCTCCGACTTGGATCTCCCCTCTCTCTGAACATGGCCAGGAAGACGCTCAACAGTCTCGAGG
ATAAGGCTTTTCAGCTCACGCCGATTGCAGTGCAAATGACAAAACTCGCCACTACAGAGGA
ACTTCCAGATGAATTTGTCGTTGTAACCGTTAAAGGAGGTTCAGGCGGGGGTGGCTCCGGC
GGGAGTGACAACGGGCCGCAAAATCAGAGAAATGCACCTCGCATAACGTTCGGAGGACCG
TCCGACTCTACCGGGAGCAACCAAAATGGGGAGCGGAGCGGTGCGCGAAGCAAACAACGA
CGGCCGCAGGGTCTGCCGAACAACACGGCTTCCTGGTTTACAGCGTTGACTCAGCATGGGA
AAGAGGACCTTAAATTCCCACGGGGGCAGGGGGTTCCTATTAACACAAATTCTAGTCCAGA
CGACCAAATCGGATATTATCGCAGAGCTACACGCAGGATTAGGGGAGGTGATGGCAAAATG
AAAGACTTGTCACCGAGGTGGTATTTTTATTACCTCGGTACAGGCCCTGAAGCTGGCCTCCC
GTATGGAGCGAATAAGGATGGCATCATTTGGGTCGCCACCGAAGGCGCTTTGAATACACCT
AAAGATCATATCGGCACAAGAAACCCCGCGAACAATGCAGCAATAGTATTGCAACTCCCTC
AGGGGACCACTTTGCCTAAAGGTTTCTACGCCGAAGGTAGCCGAGGCGGTTCACAAGCGAG
TAGTAGATCTAGCTCTCGGTCTCGGAACTCTAGTAGGAATAGCACACCTGGTTCTTCACGCG
GCACCAGCCCGGCTAGAATGGCGGGTAACGGCGGCGACGCAGCTTTGGCATTGCTGCTTCT
GGACAGACTCAACCAACTTGAATCTAAAATGAGCGGTAAGGGGCAACAGCAACAAGGGCA
AACTGTTACGAAAAAATCAGCTGCGGAAGCGTCCAAAAAACCACGACAGAAACGGACGGC
CACTAAGGCTTACAATGTGACACAAGCTTTTGGTAGACGGGGCCCTGAACAGACGCAAGGT
AACTTCGGTGATCAAGAACTGATTCGACAAGGAACAGATTACAAGCACTGGCCACAAATTG
CACAATTCGCCCCCAGCGCGTCAGCTTTCTTTGGGATGAGCCGCATTGGAATGGAAGTCACC
CCGAGCGGAACCTGGCTCACCTATACGGGGGCAATCAAACTCGATGATAAAGACCCTAATT
TCAAGGATCAGGTTATTTTGCTTAATAAGCACATAGACGCATATAAAACCTTTCCACCGACG
GAACCTAAAAAGGACAAGAAAAAAAAGGCAGATGAGACGCAAGCACTCCCTCAGAGACAA
AAGAAGCAACAGACGGTGACATTGCTCCCAGCGGCAGATTTGGATGATTTCAGTAAGCAGT
TGCAGCAATCTATGTCTTCCGCGGATTCCACTCAGGCAGGTGGGTCTTTGGGCGGCGGAGGT
TCCGGAATTGTTGGCATAGTGGCGGGCCTCGCTGTGTTGGCCGTGGTTGTCATAGGAGCAGT
CGTTGCCACGGTCATGTGTAGAAGGAAGTCATCAGGTGGGAAGGGGGGCAGTTATTCACAG
GCGGCGAGTTCCGACAGTGCGCAGGGTAGCGACGTATCACTCACTGCCTAGTAA.

The string named “RS_C3 2A linkers” (abbreviated RS C3) encodes an ORF having an amino acid length of 1326 amino acids, and the nucleotide sequence encoding the ORF is 3978 nucleotides long. The entire string is 4287 nucleotides long and encodes a peptide string that is 1429 amino acids long. Table 11 exemplifies the amino acid sequence and the nucleic acid sequences (not codon optimized) encoding the same. An exemplary codon optimized sequence for string named “RS_C3 2A linkers” is:

(SEQ ID: RS C3n)
ATGCGCGTCATGGCCCCACGAACTTTGATACTTCTGCTGTCCGGCGCATTGGCCCTT
ACGGAAACGTGGGCGGGAAGCGGCGGCTCCGGCGGAGGAGGCAGTGGCGGTTCTGATAAC
GGACCGCAAAATCAAAGGAATGCCCCGAGGATAACCTTCGGCGGACCCAGTGATTCTACCG
GCTCTAATCAGAACGGAGAACGATCCGGCGCTAGATCAAAACAACGACGACCGCAGGGGT
TGCCGAACAATACTGCGAGCTGGTTTACGGCCCTGACCCAACATGGGAAGGAAGATCTCAA
ATTTCCGCGCGGTCAAGGGGTCCCTATTAACACCAATAGTTCTCCTGACGATCAAATTGGAT
ACTACCGGAGAGCCACCCGACGAATACGCGGAGGAGATGGTAAAATGAAAGATCTTTCCCC
ACGCTGGTATTTCTATTACCTCGGGACAGGACCAGAAGCGGGATTGCCTTATGGTGCTAATA
AAGATGGTATCATTTGGGTAGCGACAGAAGGTGCGCTGAATACGCCGAAAGATCACATCGG
GACACGCAACCCAGCTAATAATGCCGCTATCGTATTGCAACTGCCCCAAGGAACAACGCTG
CCTAAGGGATTTTATGCAGAGGGAAGTCGGGGGGGGTCTCAAGCCTCATCTCGGAGCAGTT
CCCGCAGTCGAAATTCCTCTCGCAATTCCACACCAGGAAGTTCCCGAGGAACTTCACCGGC
AAGAATGGCGGGGAACGGCGGTGATGCTGCCCTTGCTCTGCTTTTGCTCGATCGCCTCAACC
AGCTTGAAAGCAAGATGTCTGGGAAGGGACAACAACAGCAGGGCCAAACGGTCACAAAGA
AAAGTGCCGCCGAAGCCTCCAAGAAACCACGACAAAAGCGGACAGCCACTAAAGCTTACA
ACGTGACTCAAGCTTTCGGTCGACGGGGCCCTGAGCAGACCCAAGGGAATTTCGGAGATCA
AGAACTGATACGGCAAGGGACGGATTACAAGCACTGGCCCCAAATTGCCCAGTTTGCTCCT
TCTGCATCTGCCTTTTTCGGTATGTCACGGATCGGAATGGAGGTAACGCCGTCCGGAACATG
GCTGACTTATACAGGAGCCATTAAACTCGACGATAAAGATCCTAACTTTAAAGATCAGGTT
ATACTGCTCAACAAACACATAGATGCATACAAAACTTTCCCCCCTACGGAACCAAAGAAGG
ATAAGAAGAAGAAAGCTGACGAGACGCAGGCCCTCCCGCAAAGACAAAAGAAACAACAGA
CTGTCACCCTGTTGCCGGCGGCTGATCTGGACGACTTCAGCAAGCAATTGCAGCAATCCATG
TCTAGTGCAGACTCCACTCAGGCCGGAAGTGGTGGTTCCGGGGAGGGTAGGGGGTCTCTCT
TGACATGTGGCGACGTGGAAGAAAACCCTGGGCCTGATCCCAAGATTTCAGAAATGCACCC
AGCTCTCAGACTGGTGGACCCTCAAATACAGCTTGCGGTAACAAGAATGGAAAACGCTGTA
GGGCGCGACCAGAATAACGTCGGGCCAAAGGTTTACCCCATAATTCTGCGACTTGGTAGTC
CTCTTTCCCTGAATATGGCGCGCAAAACACTGAATTCATTGGAGGATAAGGCGTTTCAACTT
ACGCCTATAGCAGTCCAAATGACGAAACTGGCAACCACAGAAGAACTCCCGGACGAGTTTG
TTGTAGTGACCGTTAAAGGGAGTGGGGGCAGCGGAGCCACCAACTTCTCACTCCTCAAACA
AGCAGGTGACGTCGAAGAGAATCCCGGACCCTACCACTTCTTCCACACCACTGACCCGAGC
TTCCTGGGTAGATACATGTCTGCCCTCTTTGCAGACGATTTGAATCAACTTACTGGTTACCAT
ACTGACTTTTCAAGTGAAATAATTGGCTACCAGCTCATGTGTCAACCCATCCTGCTTGCGGA
AGCGGAATTGGCCAAAAATGTGTCCCTCATACTGGGGACAGTCAGTTGGAACTTGAAGCGG
CGCTACCTCCTGTCAGCCGGTATTTTTGGGGCAATCACAGATGTATTCTACAAAGAGAACTC
ATACAAAGTGCCTACGGACAACTATATAACTACTTATGCTAGAATGGCTGCTCCAAAAGAA
ATAATTTTTCTGGAGGGCGAGACTCTCTTTGGTGACGACACGGTCATTGAGGTAGCGATAAT
ACTTGCGAGCTTCTCTGCCAGTACAAGACGAATGGCTATGGTAACGAACAACACATTTACG
TTGAAGGTGCCGCACGTTGGAGAAATCCCCGTTGCATATCGAAAAGTTTTGCTGAAAACCA
TTCAGCCTCGAGTAGAGAAATACTTGTTCGACGAATCCGGTGAGTTTAAACTGAGCGAAGT
AGGCCCCGAACACTCCCTCGCAGAATACTATATATTTTTCGCTTCCTTTTACTATAAAAGAA
ACGGTGGCAGTGGTGTCAAGCAAACCCTGAACTTCGATCTCCTCAAGTTGGCAGGGGATGT
AGAGTCTAACCCTGGTCCGGACCTTTTCATGAGGATCTTTACTATCGGTACGGTCACCCTCA
AACAAGGCGAGATAAAAGACGCCACGCCCTCAGACTTCGTGCGAGCTACTGCAACCATCCC
AATACAGGCAAGCCTGCCCTTTGGCTGGTTGATCGTCGGGGTGGCACTCCTGGCTGTGTTTC
AGAGTGCGTCAAAGATAATTACTTTGAAGAAGAGGTGGCAATTGGCACTCTCCAAAGGTGT
CCACTTTGTTTGCAATTTGCTTCTCCTGTTTGTCACCGTCTACAGCCACCTTCTGCTGGTCGC
TGCTGGCCTGGAAGCACCGTTCCTGTACCTTTATGCCTTGGTGTACTTCCTCCAGAGCATTA
ACTTTGTTAGAATCATCATGCGCTTGTGGCTGTGTTGGAAATGTCGGTCCAAGAACCCGCTC
CTCTATGATGCAAATTATTTCCTTTGTTGGCATACGAATTGCTATGACTACTGTATTCCATAT
AATTCTGTAACGTCATCAATTGTTATAACGAGCGGAGACGGTACGACCTCCCCTATTAGCGA
ACATGATTACCAAATTGGTGGCTACACCGAAAAATGGGAATCAGGAGTAAAAGACTGCGTT
GTGTTGCATAGTTATTTTACCAGTGACTATTACCAATTGTACTCAACTCAACTGAGCACTGA
CACAGGTGTGGAGCACGTTACCTTCTTCATTTACAACAAGATTGTGGACGAGCCCGAAGAA
CACGTGCAGATTCATACAATTGATGGGTCCAGTGGTGTTGTCAATCCGGTCATGGAGCCCAT
ATACGATGAGCCGACTACCACAACTTCCGTGCCGCTCGGGAGCGGCGGATCAGGACAATGC
ACAAATTACGCACTCTTGAAGTTGGCCGGTGATGTAGAAAGCAATCCTGGTCCGGCCGACA
GCAACGGAACCATAACTGTAGAGGAATTGAAAAAGCTCCTGGAACAATGGAATCTCGTGAT
CGGTTTCCTCTTCCTTACATGGATCTGTTTGCTTCAGTTTGCGTATGCTAATCGCAATCGCTT
TCTGTATATCATAAAACTTATTTTCCTTTGGCTCCTGTGGCCCGTAACGCTGGCCTGCTTCGT
GCTTGCGGCGGTATATAGAATTAACTGGATCACTGGCGGCATAGCGATCGCTATGGCATGC
TTGGTGGGGCTCATGTGGTTGAGCTACTTTATTGCATCTTTTAGATTGTTCGCGCGAACGCG
ATCCATGTGGAGTTTTAATCCTGAAACGAATATATTGCTGAATGTACCTTTGCATGGAACAA
TTTTGACGCGCCCCTTGTTGGAAAGCGAACTCGTCATAGGCGCTGTGATATTGAGGGGACAC
CTGCGGATCGCGGGTCACCACCTCGGACGATGCGATATTAAGGATCTGCCCAAAGAGATCA
CGGTAGCCACCTCCCGAACCCTGAGTTACTACAAGCTTGGTGCTAGCCAACGAGTGGCTGG
GGACTCAGGTTTCGCGGCCTATAGTCGATATCGCATCGGCAATTACAAGCTGAATACGGAC
CATTCTAGTAGTAGTGATAATATCGCTCTCCTGGTACAAGGGGGAAGCCTCGGTGGAGGGG
GATCCGGTATTGTCGGAATTGTCGCCGGTTTGGCTGTGCTGGCAGTAGTAGTGATTGGAGCA
GTTGTCGCTACTGTAATGTGCAGAAGAAAGTCCAGCGGCGGCAAAGGGGGATCTTATAGCC
AGGCGGCAAGTAGTGATTCAGCGCAGGGATCCGATGTGAGCTTGACGGCTTAGTAA

The string named “RS C4 ORF1ab as linkers” (abbreviated RS C4) encodes an ORF having an amino acid length of 1234 amino acids, and the nucleotide sequence encoding the ORF is 3702 nucleotides long. The entire string is 4011 nucleotides long and encodes a peptide string that is 1337 amino acids long. Table 11 exemplifies the amino acid sequence and the nucleic acid sequences (not codon optimized) encoding the same. An exemplary codon optimized sequence for string named “RS C4 ORF1ab as linkers” is:

(SEQ ID RS C4n)
ATGCGGGTTATGGCCCCGCGGACCCTTATCCTCCTTCTCTCAGGCGCACTTGCCTTG
ACCGAAACGTGGGCTGGGAGCGGGGGATCTGGTGGTGGGGGTTCAGGGGGCTCCGACAAT
GGACCTCAGAACCAGAGGAATGCCCCTAGAATTACTTTTGGTGGGCCTTCTGACTCCACGG
GCTCCAACCAAAACGGCGAACGATCTGGCGCCAGATCAAAGCAGCGGAGGCCTCAGGGCTT
GCCGAACAACACCGCCTCCTGGTTCACAGCCCTGACCCAGCATGGCAAGGAAGACCTCAAA
TTTCCAAGAGGCCAGGGCGTTCCAATCAACACGAATAGCAGCCCTGATGATCAAATAGGTT
ATTACAGAAGAGCCACCAGAAGGATCAGAGGAGGCGATGGGAAAATGAAGGACCTTAGCC
CAAGGTGGTACTTCTACTATCTCGGAACCGGGCCTGAAGCTGGGTTGCCTTACGGCGCCAAT
AAGGACGGTATAATATGGGTTGCTACAGAAGGGGCGCTTAACACTCCCAAAGATCATATCG
GTACGCGAAATCCCGCAAACAATGCCGCAATAGTGTTGCAGCTGCCGCAAGGAACAACGCT
CCCCAAGGGATTTTATGCAGAGGGTTCTCGGGGAGGCAGTCAGGCATCAAGCCGCTCCAGT
TCAAGATCACGAAATAGCTCTAGGAATTCTACTCCAGGCAGTTCACGAGGAACGTCTCCGG
CCCGAATGGCCGGGAATGGGGGCGATGCCGCTTTGGCGCTTTTGCTGCTGGATAGGCTCAA
CCAACTGGAGAGTAAAATGAGTGGAAAAGGCCAGCAGCAACAAGGGCAGACTGTCACTAA
GAAGTCAGCAGCCGAAGCAAGCAAGAAACCACGACAAAAGCGGACCGCGACTAAGGCATA
TAATGTAACGCAGGCCTTCGGAAGACGAGGGCCAGAGCAAACCCAAGGCAACTTCGGTGA
CCAAGAATTGATCAGGCAAGGCACCGATTATAAACATTGGCCGCAAATCGCGCAATTTGCT
CCTAGCGCAAGCGCCTTTTTCGGCATGAGCAGGATTGGCATGGAAGTCACACCAAGTGGAA
CATGGCTCACGTATACGGGGGCAATTAAACTCGATGACAAGGACCCGAATTTCAAGGATCA
AGTGATTTTGTTGAACAAGCACATAGACGCGTACAAAACTTTCCCGCCAACTGAGCCCAAG
AAGGATAAAAAAAAGAAAGCAGACGAGACACAGGCACTCCCGCAGCGACAAAAGAAACA
ACAGACGGTGACGCTTCTGCCAGCTGCCGACCTCGACGATTTCTCCAAGCAACTTCAGCAAT
CAATGTCAAGCGCAGATTCTACTCAAGCCTTTAGAGCTTGCATGGTCACTAACAATACATTT
ACACTCAAGGTACCGCATGTAGGGGAAATTCCCGTGGCCTACCGGAAGGTACTGCTCAAAA
CGATTCAACCTAGGGTAGAAAAATATCTTTTCGATGAATCAGGCGAATTTAAGCTTAGCGA
AGTGGGCCCAGAACATAGCCTCGCTGAGTATTATATTTTTTTCGCGTCCTTTTATTATAAGA
GAAATGGCGATCCCAAGATTTCAGAAATGCATCCTGCCCTTCGCCTCGTGGATCCTCAAATC
CAGCTCGCCGTTACAAGAATGGAGAACGCGGTAGGTAGAGATCAGAATAATGTTGGGCCTA
AAGTCTATCCGATTATTTTGCGGTTGGGCAGCCCCCTGAGTTTGAACATGGCTCGCAAGACC
TTGAATTCACTTGAGGACAAGGCATTCCAGCTGACGCCTATTGCGGTACAGATGACCAAGC
TGGCAACCACGGAAGAACTGCCGGATGAGTTTGTAGTCGTCACCGTAAAGTTTAACTCCTTC
CATACCACTGATCCCAGTTTTTTGGGGCGGTACATGAGTGCCCTTTTCGCGGACGATCTTAA
TCAACTCACGGGCTATCACACAGACTTTTCCAGTGAAATCATCGGGTATCAACTCATGTGTC
AGCCCATTCTGCTCGCTGAGGCTGAGCTGGCAAAGAACGTTAGCTTGATACTTGGGACGGT
GTCTTGGAACCTCAAAAAACAGGGCGATCTTTTTATGAGGATTTTTACGATTGGTACCGTAA
CGCTTAAACAAGGAGAGATTAAGGACGCAACCCCGAGTGACTTTGTCAGGGCGACAGCGAC
CATCCCTATTCAAGCAAGCCTGCCTTTTGGCTGGCTCATAGTCGGGGTCGCTCTGCTTGCTGT
ATTCCAGAGTGCCAGTAAAATCATCACTCTTAAAAAGCGATGGCAGCTGGCCCTTAGTAAG
GGGGTCCATTTCGTCTGCAACCTTCTGCTTTTGTTTGTCACCGTGTACTCTCATTTGCTCCTG
GTGGCCGCTGGACTGGAGGCTCCTTTCCTCTACCTTTACGCCCTTGTTTATTTTCTTCAATCC
ATCAATTTCGTGCGAATTATAATGCGCCTCTGGTTGTGCTGGAAGTGCCGGAGCAAAAATCC
TCTGCTCTACGATGCTAACTACTTTTTGTGTTGGCACACGAATTGCTACGACTACTGCATACC
TTACAATTCCGTGACCTCATCAATTGTGATAACGAGCGGTGACGGAACGACATCACCAATTT
CTGAGCATGACTACCAGATTGGTGGCTACACGGAAAAATGGGAATCTGGCGTCAAGGACTG
TGTGGTCCTGCATTCCTATTTTACGAGCGACTATTATCAGCTTTACTCCACGCAACTTAGTAC
GGACACCGGTGTCGAGCATGTCACGTTTTTTATTTACAATAAGATTGTTGATGAACCTGAAG
AACACGTGCAGATACATACCATTGACGGCTCTTCTGGAGTTGTGAACCCTGTCATGGAGCCT
ATCTACGACGAGCCAACAACTACGACTTCCGTACCTCTGAGAAGAAGCTACTTGTTGTCAGC
CGGGATATTCGGTGCGATCACCGACGTCTTCTATAAGGAGAATAGTTATAAGGTCCCTACA
GATAATTATATTACCACCTATGCGAGGATGGCGGCTCCTAAGGAGATTATATTCTTGGAGGG
GGAAACCCTGTTTGGCGATGACACCGTGATCGAGGTGGCCATTATACTTGCATCATTTTCTG
CCAGTACTCTCTTGGTACAGGCTGATAGTAATGGGACAATAACGGTTGAAGAACTTAAAAA
GCTTCTGGAACAGTGGAACTTGGTCATTGGATTTCTGTTCCTCACGTGGATTTGCCTCTTGCA
ATTCGCTTATGCAAATAGGAATCGGTTTCTTTATATCATCAAGTTGATATTCCTCTGGCTCCT
GTGGCCAGTGACTCTTGCTTGCTTTGTCCTGGCTGCCGTTTACCGAATAAATTGGATAACCG
GTGGTATCGCAATAGCTATGGCCTGTTTGGTGGGTCTGATGTGGTTGTCTTACTTCATAGCA
TCATTCCGCTTGTTCGCTAGAACTAGATCCATGTGGTCCTTCAACCCTGAAACTAATATTCTT
CTGAATGTGCCTCTTCACGGTACAATTTTGACACGACCACTCCTCGAAAGCGAACTTGTAAT
TGGGGCCGTGATCTTGAGGGGCCACCTTAGGATTGCAGGGCACCACTTGGGCAGATGCGAC
ATTAAGGATTTGCCAAAAGAAATAACGGTCGCGACTTCTCGGACATTGAGTTACTACAAAT
TGGGTGCATCCCAACGGGTGGCGGGTGATAGTGGGTTTGCGGCCTACTCTAGGTATCGAAT
CGGAAATTACAAGCTTAACACCGACCATTCAAGTAGTTCTGACAACATAGCTCTTCTGGTTC
AGGGTGGTTCTCTGGGTGGGGGAGGCTCCGGGATTGTCGGGATTGTCGCCGGTCTTGCTGTA
CTTGCCGTGGTGGTAATCGGAGCAGTCGTAGCTACAGTGATGTGCCGCAGAAAGAGTTCTG
GGGGTAAAGGCGGATCTTATAGCCAGGCAGCAAGCAGTGATTCCGCACAAGGATCCGATGT
GAGTCTTACCGCCTGATAA

The string named “RS C5 2300” (abbreviated RS C5) encodes an ORF having an amino acid length of 756 amino acids, and the nucleotide sequence encoding the ORF is 2268 nucleotides long. The entire string is 2577 nucleotides long and encodes a peptide string that is 859 amino acids long. Table 12 exemplifies the amino acid sequence and the nucleic acid sequences (not codon optimized) encoding the same. An exemplary codon optimized sequence for string named “RS C5 2300” is:

(SEQ ID: RS C5n)
ATGCGCGTAATGGCGCCACGAACGTTGATTCTGTTGTTGAGTGGTGCTCTCGCGCT
CACGGAGACGTGGGCCGGATCAGGAGGGAGCGGGGGTGGTGGCTCTGGTGGAAAGGACCT
TTCTCCTCGGTGGTATTTTTACTATCTGGGGACCGGTCCTGAAGCGGGGTTGCCATATGGCG
CCAACAAGGATGGCATTATCTGGGTGGCGACAGAGGGCGCGCTTAATACACCGAAGGACCA
TATAGGAACGAGAAATCCAGCGAACAATGCTGCGATTGTCCTTCAGCTGCCGCAAGGAACG
ACCTTGCCCAAGGGTTTTTACGCCGAGGGCTCTAGGGGGGGCTCCCAAGCATCATCCCGATC
CAGCTCCCGGTCTCGAAATAGCTCCCGGAATAGCACTCCTGGTTCCTCCCGGGGAACGTCCC
CAGCGCGAATGGCAGGTAACGGGGGTGACGCCGCATTGGCTCTTCTCTTGTTGGATAGACT
GAATCAGTTGGAATCAAAGATGAGCGGAAAGGGACAACAACAACAGGGACAAACCGTAAC
TAAAAAAAGCGCTGCTGAGGCGAGTAAAAAACCGAGGCAAAAGCGAACTGCTACAAAAGC
GTATAATGTCACACAAGCCTTTGGTCGCAGAGGTCCAGAGCAGACTCAGGGTAACTTCGGA
GACCAGGAGTTGATCAGACAAGGGACTGATTACAAGCACTGGCCGCAGATCGCGCAATTCG
CTCCCAGCGCGAGTGCCTTCTTCGGAATGTCAAGGATCGGAATGGAGGTCACGCCCTCAGG
CACCTGGCTGACGTACACAGGTGCAATTAAGCTGGACGATAAGGATCCCAACTTTAAGGAT
CAAGTCATCCTTCTTAACAAACACATTGATGCCTATAAAACCTTCCCGCCCACGGAGCCGAA
GAAAGATAAGAAAAAAAAAGCTGATGAGACGCAGGCGCTGCCACAAAGACAGAAGAAGC
AACAAACCGTAACCCTCCTCCCTGCAGCGGACTTGGACGACTTCAGTAAGCAACTCCAGCA
ATCCATGTCCAGTGCGGATAGTACTCAGGCGTTTCGAGCCTGTATGGTTACCAACAACACAT
TCACTCTTAAGGTGCCCCATGTTGGGGAGATCCCCGTGGCGTATAGAAAAGTACTCTTGAAA
ACGATCCAACCTCGCGTGGAGAAATACCTCTTTGACGAATCTGGGGAATTCAAACTTAGCG
AGGTAGGCCCGGAACATTCCCTCGCAGAATACTATATTTTTTTCGCTAGTTTTTACTATAAG
CGATGCTTCCATACGACAGACCCCTCTTTTCTGGGACGGTACATGTCCGCCTTGTTTGCGGA
TGACCTTAACCAATTGACGGGCTACCATACAGACTTTTCATCCGAAATAATCGGTTACCAAC
TCATGTGTCAGCCTATACTCCTCGCCGAAGCCGAGCTGGCAAAAAATGTAAGTCTGATTCTG
GGTACTGTGTCATGGAATCTGAAGAAGCGATATCTCCTTTCTGCCGGTATATTCGGTGCAAT
AACCGACGTCTTTTATAAGGAAAACAGTTACAAAGTACCGACAGACAATTATATAACCACC
TATGCACGCATGGCCGCCCCCAAAGAAATCATTTTCCTTGAGGGTGAAACGTTGTTTGGGGA
TGACACAGTTATAGAGGTGGCGATAATCCTGGCTTCATTCAGTGCTTCTACGCGACGCAGCG
GTGCTGATAGTAATGGCACAATTACTGTAGAAGAGTTGAAAAAACTGCTGGAACAATGGAA
CCTTGTTATAGGCTTTTTGTTTTTGACCTGGATATGTCTCTTGCAGTTTGCGTACGCTAATAG
GAACAGGTTCCTGTACATAATCAAGCTCATCTTCTTGTGGCTGCTTTGGCCAGTAACACTTG
CCTGTTTTGTGCTGGCCGCGGTTTATAGGATCAACTGGATAACTGGCGGGATAGCAATAGCT
ATGGCGTGTCTCGTCGGGTTGATGTGGCTGTCCTATTTTATCGCATCTTTCCGACTTTTTGCA
CGGACCAGAAGCATGTGGTCCTTTAACCCGGAGACTAATATTTTGCTCAATGTACCACTGCA
CGGGACAATACTGACACGCCCCTTGTTGGAATCTGAGTTGGTAATAGGGGCTGTAATTCTCC
GCGGTCACCTTAGGATTGCAGGTCACCACCTGGGACGCTGCGATATAAAGGATCTTCCTAA
GGAAATTACGGTAGCAACGTCACGAACTCTCAGTTATTATAAACTTGGCGCCAGTCAGCGA
GTCGCTGGCGATAGCGGATTCGCCGCGTACTCTAGATACAGAATAGGAAACTACAAATTGA
ACACGGATCACAGCTCTTCATCAGACAATATCGCCCTTCTCGTACAGGGAGGCTCACTGGG
AGGGGGCGGCAGTGGTATAGTTGGTATTGTAGCGGGCTTGGCGGTCCTTGCGGTAGTTGTTA
TAGGTGCCGTCGTCGCCACTGTCATGTGCAGGCGGAAAAGCTCTGGTGGAAAGGGCGGGAG
CTATTCACAAGCCGCGTCCTCTGACTCTGCTCAGGGTTCAGATGTTAGTCTTACAGCATGAT
AA

The string named “RS C6 1200” (abbreviated RS C6) encodes an ORF having an amino acid length of 404 amino acids, and the nucleotide sequence encoding the ORF is 1212 nucleotides long. The entire string is 1521 nucleotides long and encodes a peptide string that is 507 amino acids long. Table 12 exemplifies the amino acid sequence and the nucleic acid sequences (not codon optimized) encoding the same. An exemplary codon optimized sequence for string named “RS C6 1200” is:

(SEQ ID: RS C6n)
ATGCGCGTGATGGCACCGAGGACGCTTATTCTCTTGCTGTCAGGTGCGCTCGCCCT
CACTGAGACATGGGCAGGGTCTGGAGGTAGTGGCGGCGGTGGGAGCGGAGGAAGAATGGC
TGGGAATGGGGGCGACGCTGCGCTCGCACTCTTGCTGTTGGACCGACTGAATCAGCTCGAG
AGCAAAATGAGTGGTAAGGGGCAACAACAGATGCGCGCGTGTATGGTGACTAACAACACC
TTTACCCTTAAAGTGCCGCATGTTGGTGAAATTCCCGTTGCCTACAGAAAGGTTCTCCTTAA
AACCATTCAGCCGAGAGTCGAAAAATATTTGTTCGACGAGAGTGGTGAATTTAAACTTAGT
GAAGTAGGTCCGGAACACAGTTTGGCTGAGTATAAGATGTGCGGATATAAGCATTGGCCAC
AAATAGCCCAGTTCGCCCCTTCCGCCTCCGCCTTTTTCGGTATGTCCCGGATTGGAATGGAA
GTGACCCCATCAGGAACTTGGCTCACATACACCGGGGCTATCAAACTTGATGATAAAGATC
CAAATTTCAAAGACCAAGTTATCCTGCTGAATAAGCACATCGATGCGTACAAAACGTTCCC
CGCGCGATGCGCCACCACCGATCCCTCCTTTCTTGGTAGATATATGAGCGCGTTGTTTGCCG
ACGACCTGAACCAACTCACAGGGTACCATACGGATTTCTCATCAGAAATCATAGGTTATCA
ACTGATGTGCCAGCCAATCCTGCTGGCGGAGGCCGAGCTCGCCAAGAATGTTTCCCTTATAC
TGGGTACTGTGAGCTGGAACCTGAAAAAACAGGGGTTTGCCTATGCAAACAGGAACAGGTT
CTTGTACATCATAAAGTTGATTTTCCTTTGGTTGCTGTGGCCGGTGACCCTTGCCTGTTTTGT
ACTGGCGGCCGTCTATAGAATCAATTGGATTACCGGAGGGATTGCAATTGCTATGGCGTGTC
TTGTGGGATTGATGTGGCTCAGTTACTTCATCGCCTCATTCCGCTTGTTCTACCGCTCTTACT
TGCTGAGCGCTGGGATTTTTGGAGCAATAACAGACGTTTTCTATAAGGAAAATTCATATAAG
GTCCCAACAGATAATTACATAACCACATACGCGCGGATGGCCGCGCCTAAGGAAATTATAT
TCCTTGAGGGGGAGACGCTTTTTGGCGATGACACCGTTATCGAGGTTTCAATGTTTAATTTG
GGAAGATGTGATATTAAGGACCTTCCCAAGGAGATCACCGTTGCAACCTCACGCACGCTCA
GCTATTATAAACTTGGGGCTAGCCAGCGGGTTGCCGGGGGCAGCTTGGGTGGCGGGGGTAG
TGGTATCGTGGGAATAGTTGCTGGATTGGCCGTACTGGCTGTTGTCGTGATAGGGGCGGTAG
TAGCAACAGTTATGTGTAGACGCAAGTCCTCAGGCGGTAAAGGAGGTTCATACAGCCAGGC
GGCATCATCTGATAGTGCCCAGGGGTCCGATGTCTCACTTACCGCCTAGTAA

The string named “RS C7 1500_M_chunks” (abbreviated RS C7) encodes an ORF having an amino acid length of 492 amino acids, and the nucleotide sequence encoding the ORF is 1476 nucleotides long. The entire string is 1785 nucleotides long and encodes a peptide string that is 595 amino acids long. Table 12 exemplifies the amino acid sequence and the nucleic acid sequences (not codon optimized) encoding the same. An exemplary codon optimized sequence for string named “RS C7 1500_M_chunks” is:

(SEQ ID RS C7n)
ATGCGGGTAATGGCACCACGCACGCTGATCCTTCTTCTCTCCGGTGCACTCGCTCT
GACGGAGACGTGGGCGGGAAGTGGTGGATCAGGGGGTGGGGGGTCTGGAGGCAGTCCCGC
TAGGATGGCAGGAAATGGAGGTGATGCCGCACTGGCCCTTTTGTTGCTCGACCGGCTTAATC
AACTTGAGTCCAAGATGAGCGGCAAAGGTCAACAACAGCAAGGTCAAACTGTTACCAAAA
AGAGCGCGGCAGAAGCAAGTAAGAAGCCCCGACAAAAAAGGACTGCAACGAAGGCGTATA
ACGTAACGCAGGCCTTCGGCCGACGAGGCCCAGAACAGACACAGGGGAACTTTGGGGATC
AGGAGCTGATAAGACAAGGTACTGACTACAAACACTGGCCTCAGATTGCTCAATTCGCTCC
AAGTGCCAGTGCATTCTTCGGAATGAGCCGGATCGGGATGGAGGTAACTCCCAGCGGAACA
TGGTTGACTTACACCGGAGCGATCAAACTGGACGACAAGGACCCTAATTTCAAGGATCAGG
TTATACTGTTGAACAAACATATCGACGCGTACAAGACCTTTCCTCCCACTGAGCCTAAAAAG
GACAAGTTCAAGGCGGCCATGGTAACCAACAACACCTTTACGTTGAAAGTACCCCACGTAG
GTGAAATACCGGTGGCCTATCGAAAGGTCCTTTTGAAGACCATCCAACCCCGCGTTGAAAA
ATACCTCTTTGACGAGTCTGGTGAATTTAAACTCAGCGAAGTCGGTCCTGAGCATAGTTTGG
CGGAATATTATATTTTTTTTGCAAGTTTTTACTATAAAAGAAATGGCTTCGCGTATGCAAAT
AGAAATCGGTTTTTGTACATAATTAAGCTGATATTTCTGTGGCTCTTGTGGCCGGTCACGCT
CGCGTGCTTTGTACTCGCGGCAGTTTACAGGATCAACTGGATTACCGGAGGTATAGCAATA
GCCATGGCTTGCCTTGTTGGCCTCATGTGGCTTTCATATTTCATCGCAAGTTTTAGACTCTTC
TATAATAGTTTTCATACCACAGACCCCAGCTTCTTGGGTAGATACATGTCCGCGCTTTTTGCC
GATGACCTCAACCAGCTTACAGGGTATCACACTGACTTTTCCAGTGAAATAATTGGATATCA
GCTCATGTGTCAGCCCATCCTGCTTGCAGAAGCTGAGCTTGCGAAAAATGTCTCCCTCATCT
TGGGGACGGTTTCCTGGAATCTCAAAAAAAATGGACTGGGTCGGTGCGACATTAAGGATCT
GCCGAAAGAGATCACGGTAGCTACCAGCAGGACCCTGTCTTATTATAAGCTCGGGGCTTCC
CAACGAGTGGCTTATAGGAGTTACTTGCTGAGTGCTGGGATATTTGGTGCAATTACAGATGT
CTTCTATAAGGAGAACTCATACAAAGTACCCACTGATAACTACATAACGACCTACGCAAGA
ATGGCTGCGCCTAAAGAGATTATATTTTTGGAAGGCGAGACGTTGTTCGGTGATGATACTGT
TATAGAAGTAGCTATTATTTTGGCGAGTTTCAGCGCGAGTACTCGACGACGAGGGGGTGGT
TCTCTTGGAGGAGGAGGATCCGGGATAGTAGGCATTGTGGCTGGCTTGGCGGTGCTCGCGG
TGGTAGTAATCGGGGCTGTTGTCGCAACCGTGATGTGTCGCAGAAAATCCTCAGGTGGCAA
GGGTGGCAGCTACTCTCAAGCTGCTAGCAGCGATTCTGCCCAAGGGAGCGACGTTAGTCTT
ACGGCGTAGTAA

The string named “RS C8 1500_M_epitopes” (abbreviated RS C8) encodes an ORF having an amino acid length of 485 amino acids, and the nucleotide sequence encoding the ORF is 1455 nucleotides long. The entire string is 1764 nucleotides long and encodes a peptide string that is 588 amino acids long. Table 12 exemplifies the amino acid sequence and the nucleic acid sequences (not codon optimized) encoding the same. An exemplary codon optimized sequence for string named “RS C8 1500_M_epitopes” is:

(SEQ ID: RS C8n)
ATGAGGGTGATGGCCCCCAGGACCCTCATATTGTTGTTGAGCGGGGCCCTTGCACT
CACTGAAACTTGGGCTGGGAGTGGGGGTAGTGGTGGGGGTGGCAGTGGCGGGTCACCGGCT
CGAATGGCAGGTAACGGCGGGGATGCCGCATTGGCGCTCTTGCTGCTCGACAGGTTGAACC
AGTTGGAATCCAAGATGAGTGGAAAAGGTCAGCAACAACAGGGGCAGACCGTAACGAAGA
AGAGCGCTGCCGAGGCCTCCAAGAAGCCTCGCCAGAAACGAACGGCCACGAAGGCTTATA
ACGTGACCCAGGCATTCGGTAGAAGAGGTCCTGAACAGACACAAGGAAACTTTGGCGACCA
GGAACTCATCAGGCAAGGGACGGACTACAAGCATTGGCCGCAAATAGCGCAGTTCGCGCCC
AGCGCCAGCGCCTTCTTTGGAATGTCAAGAATTGGAATGGAAGTTACGCCGAGTGGGACTT
GGCTTACTTATACGGGCGCTATTAAGCTCGACGACAAGGACCCCAATTTCAAGGACCAAGT
GATATTGCTCAATAAGCACATCGACGCCTACAAGACTTTCCCTCCCACAGAGCCCAAAAAG
GATAAGTTCCGAGCCTGCATGGTAACGAACAATACTTTTACACTTAAGGTGCCCCATGTAGG
AGAGATACCTGTGGCTTATCGAAAAGTGCTGCTTAAGACTATTCAACCTCGCGTCGAAAAA
TACCTCTTTGACGAGAGTGGTGAGTTTAAGCTGTCAGAAGTAGGACCCGAGCATTCATTGGC
GGAGTATTACATATTCTTCGCCAGTTTTTATTATAAAAGGTGCTTCCACACGACTGACCCGT
CTTTTCTTGGTAGGTACATGAGTGCGCTGTTTGCAGACGATCTGAATCAGCTCACCGGTTAT
CACACGGATTTTAGTTCAGAAATCATAGGCTACCAGTTGATGTGCCAACCAATTCTTCTCGC
CGAGGCGGAACTTGCTAAAAATGTTAGTTTGATTTTGGGCACCGTAAGCTGGAACCTTAAA
AAACGATACCTTCTCTCTGCCGGCATATTTGGTGCTATTACTGACGTGTTCTATAAAGAGAA
TTCATATAAAGTTCCAACTGACAACTATATCACCACCTATGCTAGGATGGCGGCACCGAAG
GAAATAATCTTTCTGGAGGGGGAAACTCTGTTTGGTGATGATACCGTAATTGAGGTTGCCAT
AATACTGGCATCATTCTCAGCCAGCACTAGAAGACGCGGGAAGCTTTTGGAACAGTGGAAT
TTGGTTATTGGATTCAACCGAAACAGGTTTTTGTATATAATTAAGCTCATATTTCTTTGGTTG
TTGTGGCCCGTTACCCTTGCATGCTTCGTTCTTGCCGCCGTCTACAGTGAGCTCGTAATTGGG
GCGGTTATTCTGCGAGGACATCTCCGAATCGCTGGTCACCATCTCGGACGCACAGTCGCTAC
AAGTAGGACGCTTTCATACTATAAATTGGGAGCCAGTCAACGAGTTAAACGGTACTCAGGG
TTGATGTGGTTGAGCTATTTCGCAAGGTACGCCGGAGGATCCCTGGGAGGGGGAGGCAGCG
GTATAGTCGGTATCGTGGCAGGCCTTGCGGTGCTCGCGGTTGTAGTCATAGGCGCAGTGGTT
GCTACAGTCATGTGTCGCCGCAAATCCAGTGGAGGTAAGGGGGGTAGCTATTCCCAGGCCG
CTTCATCTGACTCAGCACAAGGATCAGACGTCTCTCTGACTGCATAATAA

The sequences as laid out are DNA sequences and can be interchangeably used for interpreting RNA (or mRNA) sequences, as is well known to one of skill in the art.

### Example 8: Pharmaceutical Composition for the Designed SARS COV-2 (2019 SARS-Cov 2) Nucleocapsid Peptide Strings

Designed strings as exemplified in Example 8, are prepared into a pharmaceutical composition of lipid nanoparticle (LNP) encapsulated mRNA having a sequence delineated in SEQ ID RS C1n, or SEQ ID RS C2n, or SEQ ID RS C3n, or SEQ ID RS C4n, or SEQ ID RS C5n, or SEQ ID RS Con, or SEQ ID RS C7n, or SEQ ID RS C8n, or any combination thereof, or sequences encoding corresponding amino acids described in Tables 11 and 12, having the amino acid sequences disclosed in column 2 of each row of sequences sets RSC1-RSC8 in Tables 11 and 12. An LNP comprise at least a cationic lipid, a non-cationic lipid and/or a PEG modified lipid. The LNP-encapsulated mRNA formulations are lyophilized and stored at a temperature below (−) 20° C., preferable frozen under liquid nitrogen for long term storage. LNP-mRNA compositions can be thawed and reconstituted in aqueous solution for use.

The LNP-mRNA compositions of the designed strings are administered alone or are co-administered with BNTSpike Vaccine for 2019 SARS CoV-2 infection.

### Example 9: Methods for Generating String Constructs

Described herein is an exemplary method for generating the string constructs by integration of bioinformatics and molecular biology techniques. Strings are designed to comprise epitopes from 2019 SARS CoV-2 proteins. These epitopes are selected based on ranking in an HLA-binding and prediction algorithm in a computer based program, as well as support from experimental data. In choosing segments of protein, the predicted population coverage based on the population HLA frequencies were taken into account in order to maximize population coverage.

Briefly the constructs are designed to incorporate from 5′-3′ top predicted and immunogenic epitopes and regions of various viral proteins, including the Nucleocapsid, Membrane, ORF3a, ORF9b and ORF1ab of the SARS CoV-2 proteins.

RNA strings were designed by concatenating sequences from different open reading frames (ORFs) of 2019 SARS-CoV-2. ORF-derived sequences could be the entire ORF; a section of the ORF (ranging in length from 99 to 954 bp-prioritized based on predicted and observed epitope density); or regions constituting or containing CD8+epitopes that were assembled to optimize for MHC class I cleavability. This last approach was taken for ORF1ab for all string variants; this was also done with the membrane ORF for one string variant (RS C8). MHC class I cleavability was scored using a neural network predictor trained on MHC class I mass spectrometry data, which takes as input a candidate peptide sequence and its context from which it should be cleaved (30 AA on either side). Four AA-long cleavability linkers (selected by testing all potential combinations of amino acids and taking the best scoring by our predictor) were added in between epitopes when efficient cleavage was not otherwise possible, and on the flanks of each region of assembled epitopes, taking into account the neighboring sequence in the given string variant. The ordering of epitopes was determined by selecting the configuration that allowed for most efficient cleavage while adding as few cleavability linkers as possible. Two string variants used putatively non-immunogenic GSS linkers to separate sequences from different ORFs. One string variant used 2A self-cleaving peptide sequences as linkers to separate sequences from different ORFs. The remaining variants used the designed MHC class I cleavable regions as ‘linkers’.

A variety of factors may be considered when determining the ordering of ORFs in a string. Within strings RS C5 to RS C8, the ordering of ORFs was driven by proteomics abundance and immunogenicity data. One string variant had the order of ORFs reversed as a control in order to evaluate translation efficiency as a function of distance along the string. In some embodiments, ordering of ORFs is driven by relative size of the ORFs (e.g., ordering from largest to smallest, with the largest ORF placed closed to the translation start site). In some embodiments, a string comprises one or more ORFs from each of M, N, and ORF1ab, where the M, N, and ORF1ab ORFs may be arranged in any order. In some embodiments, the M, N, and ORF1ab ORFs may be arranged in terms of size (e.g., from largest to smallest). In some embodiments, the M, N, and Orflab ORFs may be arranged in any of the following orders (arranged from 5′ to 3′): M, N, ORF1ab; M, ORF1ab, N; N, M ORF1ab; N, ORF1ab, M; ORF1ab, N, M; or Orflab, M, and N. In embodiments comprising a plurality of ORFs from the same antigen (e.g., two ORFs from a SARS-CoV-2 M protein), the plurality of ORFs from the same antigen may be interspersed with ORFs from other antigens (e.g., ORFs may be ordered such that a first ORF of a first antigen can be followed by an ORF of a second antigen, followed by a second ORF of the first antigen (e.g., as exemplified in strings RS-C6 and RS-C7)).

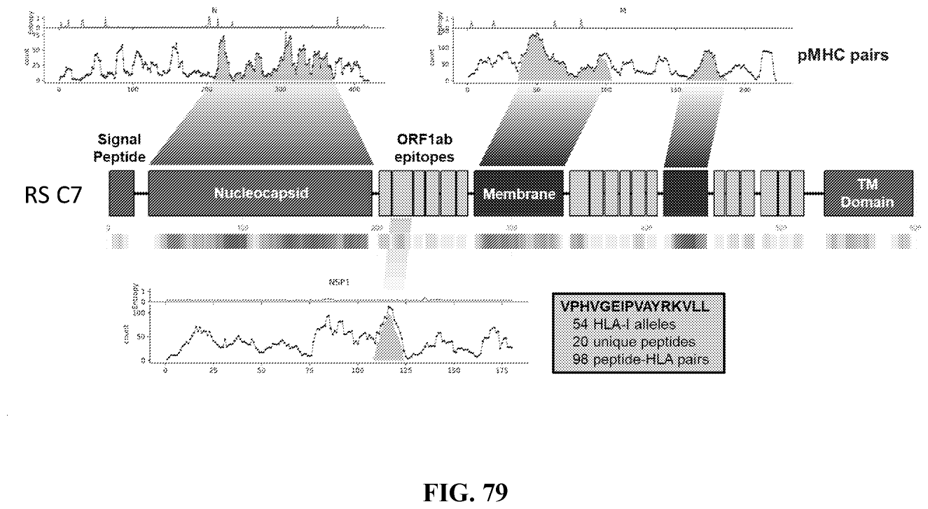

FIG. 79 depicts the C7 T-string construct and factors that were considered when designing the same. The C7 T-string construct was designed based on predicted and reported T cell antigenicity. Based on combined immunogenicity (Ferretti, Andrew P., et al. “Unbiased screens show CD8+ T cells of COVID-19 patients recognize shared epitopes in SARS-CoV-2 that largely reside outside the spike protein.” Immunity 53.5 (2020): 1095-1107; Gangaev, Anastasia, et al. “Profound CD8 T cell responses towards the SARS-CoV-2 ORF1ab in COVID-19 patients.” Research Square (2020); Gauttier, Vanessa, et al. “Tissue-resident memory CD8 T-cell responses elicited by a single injection of a multi-target COVID-19 vaccine.” BioRxiv (2020); Mateus, Jose, et al. “Selective and cross-reactive SARS-CoV-2 T cell epitopes in unexposed humans.” Science 370.6512 (2020): 89-94; Annika, N. “SARS-CoV-2 T-cell epitopes define heterologous and COVID-19-induced T-cell recognition.” Research Square, DOI 10 (2020); Dong, Tao, et al. “Broad and strong memory CD4+ and CD8+ T cells induced by SARS-CoV-2 in UK convalescent COVID-19 patients.” (2020); Sekine, Takuya, et al. “Robust T cell immunity in convalescent individuals with asymptomatic or mild COVID-19.” Cell 183.1 (2020): 158-168; Snyder, Thomas M., et al. “Magnitude and dynamics of the T-cell response to SARS-CoV-2 infection at both individual and population levels.” MedRxiv (2020); and Tarke, Alison, et al. “Comprehensive analysis of T cell immunodominance and immunoprevalence of SARS-CoV-2 epitopes in COVID-19 cases,” Cell Reports Medicine 2.2 (2021): 100204, the contents of each of which is incorporated by reference herein in their entirety) and protein expression data from infected cells (Poran, A., Harjanto, D., Malloy, M. et al, Sequence-based prediction of SARS-CoV-2 vaccine targets using a mass spectrometry-based bioinformatics predictor identifies immunogenic T cell epitopes. Genome Med 12, 70 (2020)), N, M, and nonstructural proteins (NSP) 1-4 from ORF1ab were selected for inclusion in the T cell vaccine. For the N protein, a block of 167 amino acids was selected that includes 155 unique predicted peptide epitopes from 74 unique HLA-I alleles for a total of 525 unique peptide-HLA-I pairs. For the M protein, two blocks were selected with 67 and 33 amino acids, respectively. Together, these two blocks include 148 unique predicted peptide epitopes from 66 unique HLA-I alleles for a total of 602 unique peptide-HLA-I pairs. Since ORF1ab is the longest SARS-CoV-2 ORF and comprises highly immunogenic hotspots throughout, for this protein, the T-string was designed to comprise a greater number of shorter minimal HLA-I-targeting epitopes from throughout the genomic region encoding NSPs 1-4. NSPs 1~4 were specifically chosen because they are amongst the first proteins to be synthesized upon viral infection, and that are present at a greater abundance than other ORF1ab NSPs (Bouhaddou et al., 2020; Weingarten-Gabbay et al., 2021). Considering only predicted epitopes that also demonstrate immunogenicity in the literature, 16 sequence segments of 9-16 amino acids were selected for inclusion in the vaccine. These 16 segments include 113 unique predicted peptide epitopes from 54 unique alleles for a total of 524 unique peptide-HLA-I pairs. Altogether, the antigenic regions selected for inclusion in the vaccine are predicted to cover HLA-I alleles present in 99% or more of the global population.

### Example 10: Epitope Specificity of mRNA Vaccine

An exemplary study is illustrated in this example, demonstrating T cell specificity in vivo upon administration of a BNT mRNA vaccine directed against SARS CoV-2. Study participants received a priming immunization with BNT162b2 on day 1, and a booster immunization on day 22±2. Serum was obtained on days 1 (pre-prime), 8±1 (post-prime), 22±2 (pre-boost), 29±3, 43±4, 50±4 and 85±7 (post-boost). PBMCs were obtained on days 1 (pre-prime) and 29±3 (post-boost).

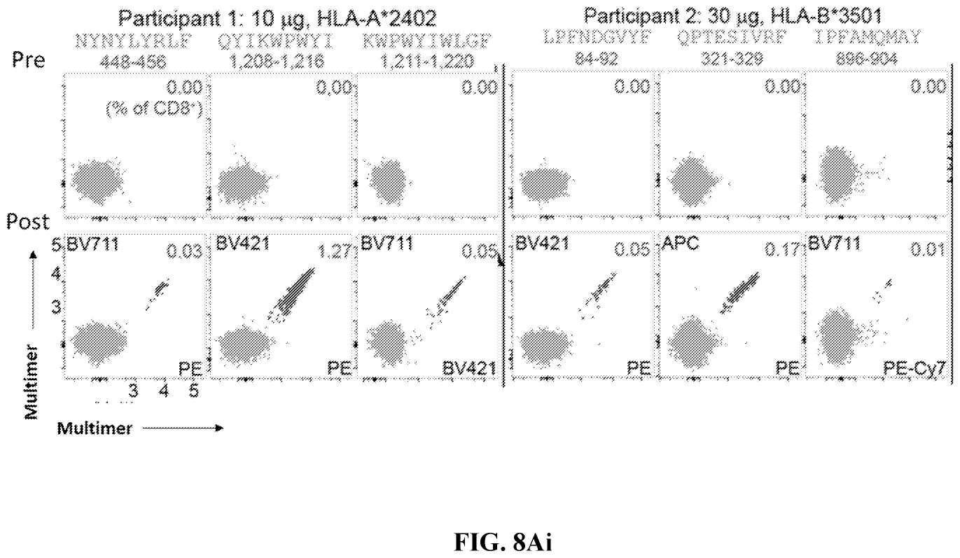

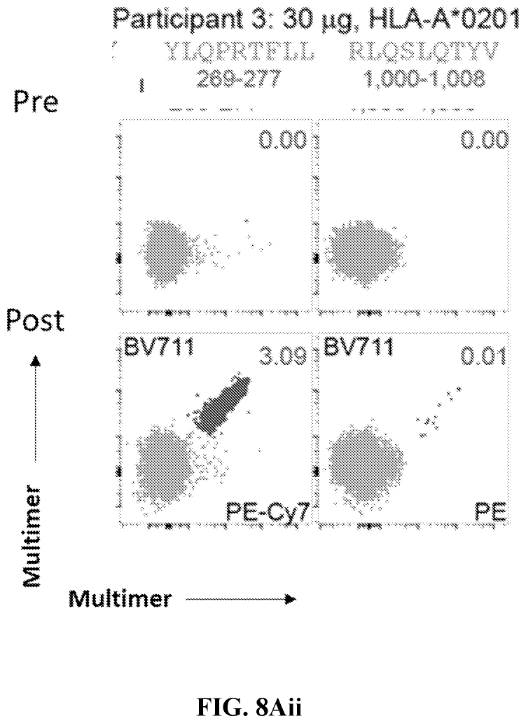

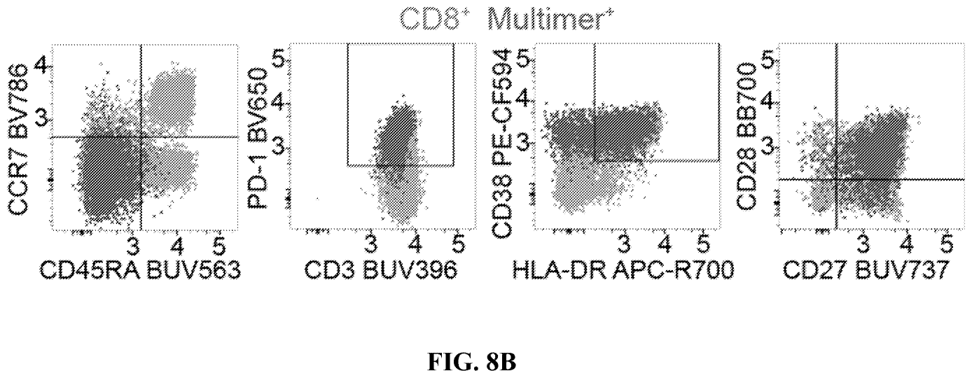

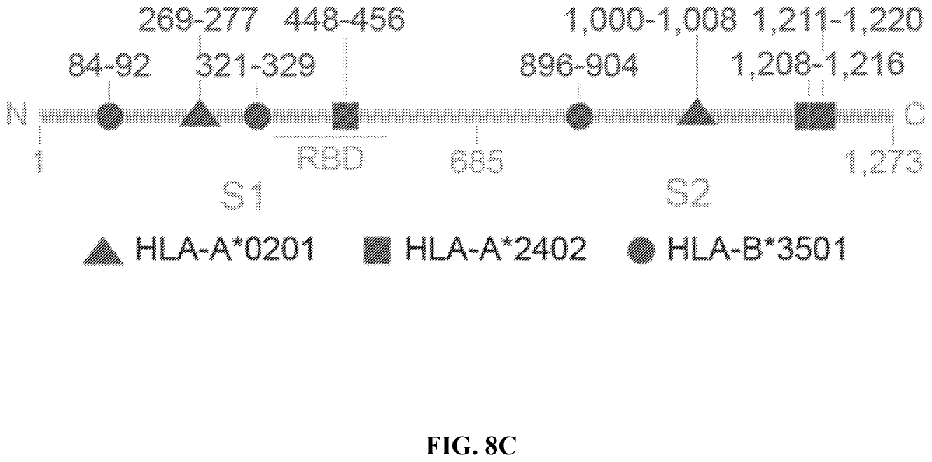

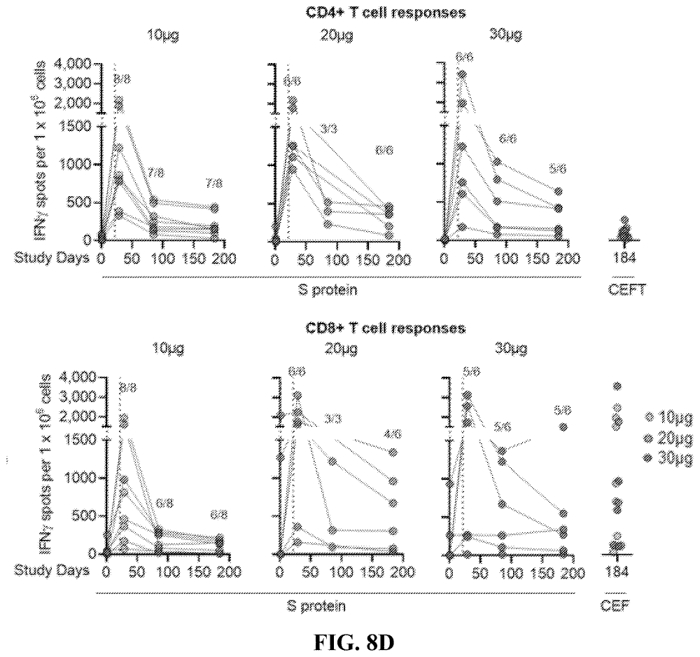

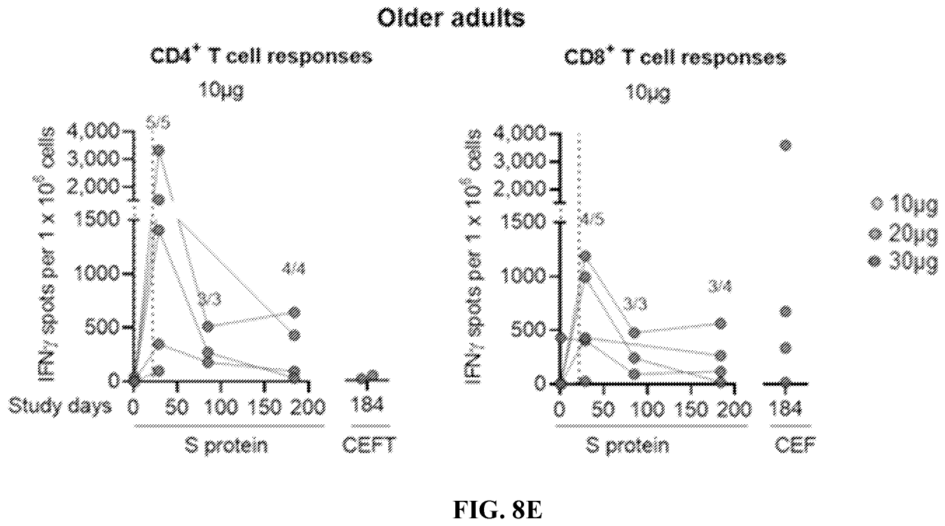

In this study, CD8+ T cell responses were characterized on the epitope level in three BNT mRNA vaccinated participants. PBMCs obtained on day 1 (pre-prime) and day 29 (7 days post-boost) of three vaccinated participants (dose cohorts 10 μg, n=1; 30 μg, n=2) were stained with individual pMHC class I multimer cocktails and analysed for T cell epitope specificity (FIG. 8Ai and 8Aii) and phenotype (FIG. 8B; example from participant 3; YLQPRTFLL) by flow cytometry. In FIG. 8Ai-8C, peptide sequences above dot plots indicate pMHC class I multimer epitope specificity, numbers above dot plots indicate the amino acids corresponding to the epitope within S protein. FIG. 8C shows localization of identified MHC class I-restricted epitopes within S protein. Pre- and post-vaccination PBMCs were stained with individualized peptide/MHC multimer staining cocktails for flow cytometry analysis. Twenty-three (4 for HLA-B*0702, 19 for HLA-A*2402), 14 (HLA-B*3501) and 23 (7 for HLA-B*4401, 16 for HLA-A*0201) diverse peptide/MHC allele pairs were used for participants 1, 2 and 3, respectively, thus probing a selected set of potential reactivities rather than comprehensively capturing the poly-epitopic T cell response. For each participant, de novo induced CD8+ T cell reactivities against multiple epitopes were identified adding up to a total of eight different epitope/MHC pairs spread across the full length of S (FIGS. 8Ai-8Aii, 8C). The magnitude of epitope-specific T cell responses ranged between 0.01-3.09% of peripheral CD8⁺ T cells, and the most profound expansion was observed for HLA-A*0201 YLQPRTFLL (3.09% multimer⁺of CD8⁺), HLA-A*2402 QYIKWPWYI (1.27% multimer⁺of CD8⁺) and HLA-B*3501 QPTESIVRF (0.17% multimer⁺ of CD8⁺). Comparison with the bulk IFNγ+ CD8+ T cell response against full S in these individuals determined by ELISpot and ICS indicated that indicated that pMHC technology may be more useful to assess true extent of the cellular immune response. Phenotyping of the identified pMHC multimer⁺ S antigen-experienced CD8+ T cell specificities revealed an early differentiated effector memory phenotype characterized by low expression of CCR7 and CD45RA and high expression of the costimulatory molecules CD28 and CD27. CD8+ T cells also expressed markers associated with cognate activation, such as CD38, HLA-DR and PD-1 (FIG. 8B). The data presented herein is the first demonstration of epitopes recognized by COVID-19 vaccine-induced T cells.

A time-course of detecting activated T cells following vaccine administration was followed in human subjects. In the study depicted in FIG. 8D, subjects were administered 10, 20 or 30 micrograms of the mRNA vaccine encoding the spike protein. Activated CD4+ T cells and CD8+ T cells were isolated at time intervals up to 200 days, and IFN gamma release from the cells was detected by ELISPOT. The subjects exhibited a longer persistence of activated both CD4+ and CD8+ T cells determined by IFNg release in a dose dependent manner. The figures also indicate that the cells were responsive to most of the epitopes that the subjects were exposed to, e.g., 7 out of 8 in the 10 microgram group at 200 days in the upper panel, left graph. The same study in an elderly cohort also showed longer persistent vaccinated epitope responsive activated T cells over the course of 200 days (FIG. 8E). Incidentally, the unrelated viral pool epitope controls (CEFT or CEF) showed lower response for CD4+ T cells. The respective epitopes, matching HLA and the response in subjects are listed below:

| | | Position in | Identified in |
|---|---|---|---|
| HLA | Epitope | S Protein | No. Subjects |
| B35:01 | LPFNDGVYF | 84-92 | 1 |
| A03:01 | GVYFASTEK | 89-97 | 1 |
| A02:01 | YLQPRTFLL | 269-277 | 3 |
| B35:01 | QPTESIVRF | 321-329 | 1 |
| A26:01 | CVADYSVLY | 361-369 | 1 |
| B15:01 | CVADYSVLY | 361-369 | 1 |
| A03:01 | KCYGVSPTK | 378-386 | 2 |
| A24:02 | NYNYLYRLF | 448-456 | 3 |
| B15:01 | FQPTNGVGY | 497-505 | 1 |
| B35:01 | IPFAMQMAY | 896-904 | 1 |
| A02:01 | RLQSLQTYV | 1000-1008 | 2 |
| A68:01 | GTHWFVTQR | 1099-1108 | 1 |
| C04:01 | VYDPLQPEL | 1137-1145 | 1 |
| A24:02 | QYIKWPWYI | 1208-1216 | 3 |
| A24:02 | KWPWYIWLGF | 1211-1220 | 1 |

### Example 11. Peptide/MHC Multimer Staining

In order to select MHC-class I epitopes for multimer analysis, a mass spectrometry-based binding and presentation predictor (e.g., as described in Abelin et al., Immunity 46, 315-326 (2017); and Poran et al., Genome Med. 12, 70 (2020)) was applied to 8-12 amino acid long peptide sequences from the Spike glycoprotein derived from the GenBank reference sequence for SARS CoV-2 (accession: NC_045512.2, ncbi.nlm.nih.gov/nuccore/NC_045512) and paired with 18 MHC-class-I alleles with >5% frequency in the European population. Top predicted epitopes were identified by setting thresholds to the binding percent-rank (≤1%) and presentation scores (>10-2.2) and considered for synthesis of peptides of >90% purity. pMHC complexes were refolded with the easYmer technology (easYmer® kit, ImmuneAware Aps), and complex formation was validated in a bead-based flow cytometry assay according to the manufacturer's instructions. Combinatorial labeling was used for dissecting the antigen specificity of T cells utilizing two-color combinations of five different fluorescent labels to enable detection of up to ten different T cell populations per sample. For tetramerisation, streptavidin (SA)-fluorochrome conjugates were added: SA BV421, SA BV711, SA PE, SA PE-Cy7, SA APC (all BD Biosciences). For three BNT162b2 vaccinated participants, individualized pMHC multimer staining cocktails contained up to ten pMHC complexes, with each pMHC complex encoded by a unique two-color combination. PBMCs (2×106) were stained ex vivo for 20 minutes at room temperature with each pMHC multimer cocktail at a final concentration of 4 nM in Brilliant Staining Buffer Plus (BSB Plus [BD Horizon™]). Surface and viability staining was carried out in flow buffer (DPBS [Gibco] with 2% FBS [Biochrom], 2 mM EDTA [Sigma-Aldrich]) supplemented with BSB Plus for 30 minutes at 4° C. (CD3 BUV395, 1:50; CD45RA BUV563, 1:200; CD27 BUV737, 1:200; CD8 BV480, 1:200; CD279 BV650, 1:20; CD197 BV786, 1:15; CD4 BB515, 1:50; CD28 BB700, 1:100; CD38 PE-CF594, 1:600; HLA-DR APC-R700, 1:150; all BD Biosciences; DUMP channel: CD14 APC-eFluor780, 1:100; CD16 APC-eFluor780, 1:100; CD19 APC-eFluor780, 1:100; fixable viability dye eFluor780, 1:1,667; all ThermoFisher Scientific). Cells were fixed for 15 minutes at 4° C. in 1x Stabilization Fixative (BD), acquired on a FACSymphony™ A3 flow cytometer (BD Biosciences) and analyzed with FlowJo software version 10.6.2 (FlowJo LLC, BD Biosciences). CD8+ T cell reactivities were considered positive, when a clustered population was observed that was labelled with only two pMHC multimer colors.

### Example 12. T Cell Manufacturing

Provided herein is a T cell therapy where T cells primed and responsive against antigenic peptides specific for a viral epitope is administered to the subject. The therapeutic can comprise generating viral epitope specific T cells ex vivo by priming T cells with APCs expressing viral T cell epitopes and expanding the activated T cells to obtain viral epitope-specific CD8+ and CD4+including a population of these cells exhibiting memory phenotype (see, e.g., WO2019094642, incorporated by reference in its entirety). Target viral antigen responsive T cells are generated ex vivo and immunogenicity is validated using an in vitro antigen-specific T cell assay. Mass spectrometry can be used to validate that cells that express the antigen of interest can process and present the peptides on the relevant HLA molecules. Additionally, the ability of these T cells to kill cells presenting the peptide is confirmed using a cytotoxicity assay.

### Generation of Target Tumor Cell Antigen Responsive T Cells Ex Vivo

### Materials:

AIM V media (Invitrogen) Human FLT3L, preclinical CellGenix #1415-050 Stock 50 ng/μL; TNF-α, preclinical CellGenix #1406-050 Stock 10 ng/μL; IL-1B, preclinical CellGenix #1411-050 Stock 10 ng/μL; PGE1 or Alprostadil-Cayman from Czech republic Stock 0.5 μg/μL; R10 media-RPMI 1640 glutamax+10% Human serum+1% PenStrep; 20/80 Media-18% AIM V+72% RPMI 1640 glutamax+10% Human Serum+1% PenStrep; IL7 Stock 5 ng/μL; IL15 Stock 5 ng/μL.

### Procedure:

Step 1: Plate 5 million PBMCs (or cells of interest) in each well of 24 well plate with FLT3L in 2 mL AIM V media
Step 2: Peptide loading and maturation—in AIMV
1. Mix peptide pool of interest (except for no peptide condition) with PBMCs (or cells of interest) in respective wells.
2. Incubate for 1 hr.
3. Mix Maturation cocktail (including TNF-α, IL-1B, PGE1, and IL-7) to each well after incubation.
Step 3: Add human serum to each well at a final concentration of 10% by volume and mix.
Step 4: Replace the media with fresh RPMI+10% HS media supplemented with IL7+IL15.
Step 5: Replace the media with fresh 20/80 media supplemented with IL7+IL15 during the period of incubation every 1-6 days.
Step 6: Plate 5 million PBMCs (or cells of interest) in each well of new 6-well plate with FLT3L in 2 ml AIM V media
Step 7: Peptide loading and maturation for re-stimulation-(new plates)
1. Mix peptide pool of interest (except for no peptide condition) with PBMCs (or cells of interest) in respective wells
2. Incubate for 1 hr.
3. Mix Maturation cocktail to each well after incubation
Step 8: Re-stimulation:
1. Count first stimulation FLT3L cultures and add 5 million cultured cells to the new Re-stimulation plates.
2. Bring the culture volume to 5 mL (AIM V) and add 500 μL of Human serum (10% by volume)
Step 9: Remove 3 ml of the media and add 6 ml of RPMI+10% HS media supplemented with IL7+IL15.
Step 10: Replace 75% of the media with fresh 20/80 media supplemented with IL7+IL15.
Step 11: Repeat re-stimulation if needed.

### Analysis of Antigen-Specific Induction

MHC tetramers are purchased or manufactured on-site according to methods known by one of ordinary skill and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a LSR Fortessa (Becton Dickinson) instrument and are analyzed by use of FlowJo software (Becton Dickinson). For analysis of tetramer positive cells, the lymphocyte gate is taken from the forward and side-scatter plots. Data are reported as the percentage of cells that were CD8⁺/tetramer⁺.

### Evaluation of Presentation of Viral Antigens

The affinity of the viral epitopes for HLA alleles and stability of the viral epitopes with the HLA alleles can be determined. An exemplary detailed description of the protocol utilized to measure the binding affinity of peptides to Class I MHC has been published (Sette et al, Mol. Immunol. 31 (11): 813-22, 1994). In brief, MHCI complexes were prepared and bound to radiolabeled reference peptides. Peptides were incubated at varying concentrations with these complexes for 2 days, and the amount of remaining radiolabeled peptide bound to MHCI was measured using size exclusion gel-filtration. The lower the concentration of test peptide needed to displace the reference radiolabeled peptide demonstrates a stronger affinity of the test peptide for MHCI. Peptides with affinities to MHCI <50 nM are generally considered strong binders while those with affinities <150 nM are considered intermediate binders and those <500 nM are considered weak binders (Fritsch et al, 2014).

An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts.

To assess whether antigens could be processed and presented from the larger polypeptide context, peptides eluted from HLA molecules isolated from cells expressing the genes of interest were analyzed by tandem mass spectrometry (MS/MS).

For analysis of presentation of viral antigens, cell lines are utilized that have been infected with the virus or were lentivirally transduced to express the viral antigens. HLA molecules are either isolated based on the natural expression of the cell lines or the cell lines are lentivirally transduced or transiently transfected to express the HLA of interest. 293T cells are transduced with a lentiviral vector encoding various regions of a viral polypeptides. Greater than 50 million cells expressing peptides encoded by a viral polypeptide are cultured and peptides were eluted from HLA-peptide complexes using an acid wash. Eluted peptides are then analyzed by targeted MS/MS with parallel reaction monitoring (PRM).

### HLA Class I Binding and Stability

A subset of the peptides used for affinity measurements are also used for stability measurements using the assay described. Less than 50 nM can be considered by the field as a strong binder, 50-150 nM can be considered an intermediate binder, 150-500 nM can be considered a weak binder, and greater than 500 nM can be considered a very weak binder.

Immunogenicity assays are used to test the ability of each test peptide to expand T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with each peptide for 1 hour before addition of a cytokine maturation cocktail (GM-CSF, IL-1B, IL-4, IL-6, TNFα, PGE1β). Cells are incubated at 37° C. to mature DCs.

### Assessment of Cytotoxic Capacity of Antigen-Specific T Cells In Vitro

Cytotoxicity activity can be measured with the detection of cleaved Caspase 3 in target cells by Flow cytometry. Target cancer cells are engineered to express the viral peptide along and the proper MHC-I allele. Mock-transduced target cells (i.e. not expressing the viral peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cells. The percentage of specific lysis is calculated as: Experimental cleavage of Caspase 3/spontaneous cleavage of Caspase 3 (measured in the absence of mutant peptide expression)×100.

In some examples, cytotoxicity activity is assessed by co-culturing induced T cells with a population of viral antigen-specific T cells with target cells expressing the corresponding HLA, and by determining the relative growth of the target cells, along with measuring the apoptotic marker Annexin V in the target cells specifically. Target cells are engineered to express the viral peptide or the viral peptide is exogenously loaded. Mock-transduced target cells (i.e. not expressing the viral peptide), target cells loaded with viral peptides, or target cells with no peptide loaded are used as a negative control. The cells are also transduced to stably express GFP allowing the tracking of target cell growth. The GFP signal or Annexin-V signal are measured over time with an IncuCyte S3 apparatus. Annexin V signal originating from effector cells is filtered out by size exclusion. Target cell growth and death is expressed as GFP and Annexin-V area (mm2) over time, respectively.

### Enrichment of Target Antigen Activated T Cells

Viral antigen responsive T cells may be further enriched. In this example, multiple avenues for enrichment of antigen responsive T cells are explored. After the initial stimulation of viral antigen-specific T cells, an enrichment procedure can be used prior to further expansion of these cells. As an example, stimulated cultures and pulsed with the same viral peptides used for the initial stimulation on day 13, and cells upregulating 4-1BB are enriched using Magnetic-Assisted Cell Separation (MACS; Miltenyi). These cells can then be further expanded, for example, using anti-CD3 and anti-CD28 microbeads and low-dose IL-2.

### Immunogenicity Assays for Selected Peptides

After maturation of DCs, PBMCs (either bulk or enriched for T cells) are added to mature dendritic cells with proliferation cytokines. Cultures are monitored for viral peptide-specific T cells using a combination of functional assays and/or tetramer staining. Parallel immunogenicity assays with the viral peptides allowed for comparisons of the relative efficiency with which the peptides expanded peptide-specific T cells. In some embodiments, the peptides elicit an immune response in the T cell culture comprises detecting an expression of a FAS ligand, granzyme, perforins, IFN, TNF, or a combination thereof in the T cell culture.

Immunogenicity can be measured by a tetramer assay. MHC tetramers are purchased or manufactured on-site, and are used to measure peptide-specific T cell expansion in the immunogenicity assays. For the assessment, tetramer is added to 1×10\hat{ }5 cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4 degrees Celsius for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/Tetramer⁺.

Immunogenicity can be measured by intracellular cytokine staining. In the absence of well-established tetramer staining to identify viral antigen-specific T cell populations, antigen-specificity can be estimated using assessment of cytokine production using well-established flow cytometry assays. Briefly, T cells are stimulated with the viral peptide of interest and compared to a control. After stimulation, production of cytokines by CD4+ T cells (e.g., IFNγ and TNFα) are assessed by intracellular staining. These cytokines, especially IFNγ, used to identify stimulated cells.

In some embodiments the immunogenicity is measured by measuring a protein or peptide expressed by the T cell, using ELISpot assay. Peptide-specific T cells are functionally enumerated using the ELISpot assay (BD Biosciences), which measures the release of IFNγ from T cells on a single cell basis. Target cells are pulsed with 10 μM viral peptide for one hour at 37 degrees C., and washed three times. 1×10\hat{ }5 peptide-pulsed targets are co-cultured in the ELISPOT plate wells with varying concentrations of T cells (5×10\hat{ }2 to 2×10\hat{ }3) taken from the immunogenicity culture. Plates are developed according to the manufacturer's protocol, and analyzed on an ELISPOT reader (Cellular Technology Ltd.) with accompanying software. Spots corresponding to the number of IFN gamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

CD107a and CD107b are expressed on the cell surface of CD8+ T cells following activation with viral peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate viral peptide-specific T cells. To perform the assay, peptide is added to HLA-transfected cells to a final concentration of 20 μM, the cells are incubated for 1 hour at 37 degrees C., and washed three times. 1×10\hat{ }5 of the peptide-pulsed cells were aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10\hat{ }5 T cells from the immunogenicity culture are added next, and the samples were incubated for 4 hours at 37 degrees C. The T cells are further stained for additional cell surface molecules such as CD8 and acquired on a FACS Calibur instrument (Becton Dickinson). Data is analyzed using the accompanying Cellquest software, and results are reported as the percentage of CD8+CD107 a and b+ cells.

Cytotoxic activity is measured using a chromium release assay. Target T2 cells are labeled for 1 hour at 37 degrees C. with Na51Cr and washed 5×10\hat{ }3 target cells are then added to varying numbers of T cells from the immunogenicity culture. Chromium release is measured in supernatant harvested after 4 hours of incubation at 37 degrees C. The percentage of specific lysis is calculated as: Experimental release−spontaneous release/Total release−spontaneous release×100

Immunogenicity assays are carried out to assess whether each peptide can elicit a T cell response by viral antigen-specific expansion. A positive result demonstrates that a peptide can induce a T cell response. Several viral peptides are tested for their capacity to elicit CD8+ T cell responses with multimer readouts as described. Each positive result was measured with a second multimer preparation to avoid any preparation biases. In an exemplary assay, T cells were co-cultured with monocyte-derived dendritic cells loaded with viral epitope for 10 days. CD8+ T cells were analyzed for viral antigen-specificity for viral epitope using multimers (initial: BV421 and PE; validation: APC and BUV396).

While antigen-specific CD8+ T cell responses are readily assessed using well-established HLA Class I multimer technology, CD4+ T cell responses require a separate assay to evaluate because HLA Class II multimer technology is not well-established. In order to assess CD4+ T cell responses, T cells are re-stimulated with the viral peptide of interest. After stimulation, production of cytokines by CD4+ T cells (e.g., IFNγ and TNFα) are assessed by intracellular staining. These cytokines, especially IFNγ, used to identify stimulated cells.

### Cell Expansion and Preparation

To prepare APCs, the following method is employed (a) obtain of autologous immune cells from the peripheral blood of the patient; enrich monocytes and dendritic cells in culture; load viral peptides and mature DCs.

### T Cell Induction (Protocol 1)

First induction: (a) Obtaining autologous T cells from an apheresis bag; (b) Depleting CD25+ cells and CD14+ cells, alternatively, depleting only CD25+ cells; (c) Washing the peptide loaded and mature DC cells, resuspending in the T cell culture media; (d) Incubating T cells with the matured DC. Second induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

Third induction: (a) Washing T cells, and resuspending in T cell media, and optionally evaluating a small aliquot from the cell culture to determine the cell growth, comparative growth and induction of T cell subtypes and viral antigen specificity and monitoring loss of cell population; (b) Incubating T cells with mature DC.

To harvest peptide activated t cells and cryopreserve the T cells, the following method can be employed (a) Washing and resuspension of the final formulation comprising the activated T cells which are at an optimum cell number and proportion of cell types that constitutes the desired characteristics of the Drug Substance (DS). The release criteria testing include inter alia, Sterility, Endotoxin, Cell Phenotype, TNC Count, Viability, Cell Concentration, Potency; (b) Filling drug substance in suitable enclosed infusion bags; (c) Preservation until time of use.

### Methods of Functional Characterization of the CD4+ and CD8+Viral Antigen-Specific T Cells.

T cell manufacturing processes were developed to raise memory and de novo CD4+ and CD8+T cell responses to viral antigens through multiple rounds of ex-vivo T cell stimulation, generating a viral antigen-reactive T cell product for use in adoptive cell therapy. Detailed characterization of the stimulated T cell product can be used to test the many potential variables these processes utilize. To probe T cell functionality and/or specificity, an assay was developed to simultaneously detect viral antigen-specific T cell responses and characterize their magnitude and function. This assay employs the following steps. First T cell-APC co-cultures were used to elicit reactivity in viral antigen-specific T cells. Optionally, sample multiplexing using fluorescent cell barcoding is employed. To identify viral antigen-specific CD8+ T cells and to examine T cell functionality, staining of peptide-MHC multimers and multiparameter intracellular and/or cell surface cell marker staining were probed simultaneously using FACS analysis. The results of this streamlined assay demonstrated its application to study T cell responses induced from a healthy donor. Viral antigen-specific T cell responses induced toward peptides are identified in a donor. The magnitude, specificity and functionality of the induced T cell responses are also compared. Briefly, different T cell samples are barcoded with different fluorescent dyes at different concentrations (see, e.g., Example 19). Each sample receives a different concentration of fluorescent dye or combination of multiple dyes at different concentrations. Samples are resuspended in phosphate-buffered saline (PBS) and then fluorophores dissolved in DMSO (typically at 1:50 dilution) are added to a maximum final concentration of 5 μM. After labeling for 5 min at 37° C., excess fluorescent dye is quenched by the addition of protein-containing medium (e.g. RPMI medium containing 10% pooled human type AB serum). Uniquely barcoded T cell cultures are challenged with autologous APC pulsed with the viral antigen peptides as described above.

The differentially labeled samples are combined into one FACS tube or well, and pelleted again if the resulting volume is greater than 100 μL. The combined, barcoded sample (typically 100 μL) is stained with surface marker antibodies including fluorochrome conjugated peptide-MHC multimers. After fixation and permeabilization, the sample is additionally stained intracellularly with antibodies targeting TNF-α and IFN-γ.

The cell marker profile and MHC tetramer staining of the combined, barcoded T cell sample are then analyzed simultaneously by flow cytometry on flow cytometer. Unlike other methods that analyze cell marker profiles and MHC tetramer staining of a T cell sample separately, the simultaneous analysis of the cell marker profile and MHC tetramer staining of a T cell sample described in this example provides information about the percentage of T cells that are both viral antigen specific and that have increased cell marker staining. Other methods that analyze cell marker profiles and MHC tetramer staining of a T cell sample, separately determine the percentage of T cells of a sample that are viral antigen specific, and separately determine the percentage of T cells that have increased cell marker staining, only allowing correlation of these frequencies.

The simultaneous analysis of the cell marker profile and MHC tetramer staining of a T cell sample described in this example does not rely on correlation of the frequency of viral antigen specific T cells and the frequency of T cells that have increased cell marker staining; rather, it provides a frequency of T cells that are both viral antigen specific and that have increased cell marker staining. The simultaneous analysis of the cell marker profile and MHC tetramer staining of a T cell sample described in this example allows for determination on a single cell level, those cells that are both viral antigen specific and that have increased cell marker staining.

To evaluate the success of a given induction process, a recall response assay may be used followed by a multiplexed, multiparameter flow cytometry panel analysis. A sample taken from an induction culture is labeled with a unique two-color fluorescent cell barcode. The labeled cells are incubated on viral antigen-loaded DCs or unloaded DCs overnight to stimulate a functional response in the viral antigen-specific cells. The next day, uniquely labeled cells are combined prior to antibody and multimer staining. Exemplary materials for T cell culture are provided below:

Materials: AIM V media (Invitrogen) Human FLT3L; preclinical CellGenix #1415-050 Stock 50 ng/μL TNFα; preclinical CellGenix #1406-050 Stock 10 ng/μL; IL-1β, preclinical CellGenix #1411-050 Stock 10 ng/μL; PGE1 or Alprostadil—Cayman from Czech republic Stock 0.5 μg/μL; R10 media-RPMI 1640 glutamax+10% Human serum+1% PenStrep; 20/80 Media-18% AIM V+72% RPMI 1640 glutamax+10% Human Serum+1% PenStrep; IL7 Stock 5 ng/μL; IL15 Stock 5 ng/μL; DC media (Cellgenix); CD14 microbeads, human, Miltenyi #130-050-201, Cytokines and/or growth factors, T cell media (AIM V+RPMI 1640 glutamax+serum+PenStrep), Peptide stocks-1 mM per peptide viral peptides).

### Example 13. Designing Linkers of Orflab Minimal Epitopes

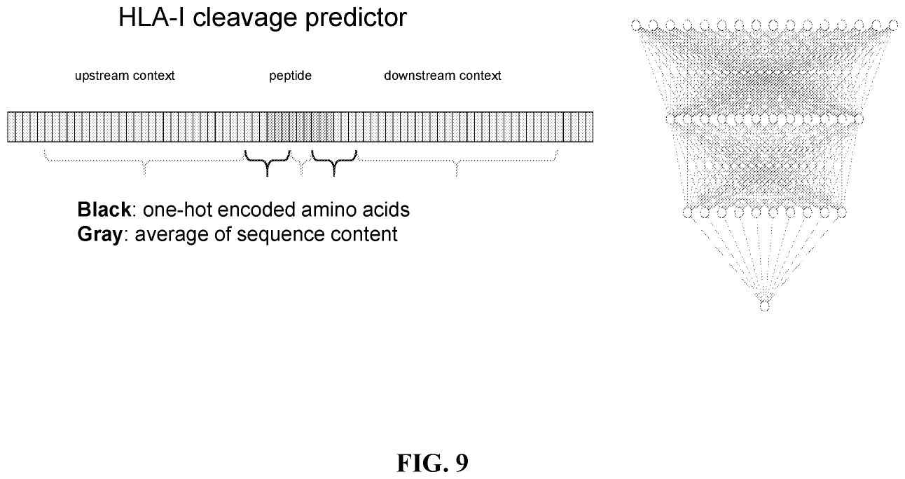

This example shows that the strings were designed using the MS-based HLA-I cleavage predictor to optimize ordering of Orflab epitopes or minimal epitope containing stretches, adding as few linkers as possible while retaining efficient epitope cleavage (FIG. 9). The same 18 Orflab sequences is included in each string variant, except RS-C6 where two sequences have been removed. However, the ordering of the sets of sequences and designed cleavage linkers differ based on the flanking context. The string RS C8 was especially designed such that the minimal Orflab minimal epitope sequences are placed in close proximity with each other, utilizing MS-based cleavage predictor on either side of an epitope and requiring minimal linker sequences.

TABLE 13
Exemplary Orf1ab epitopes optimized for cleavage

| Peptide | Start | End | Length | Score | Seq_type |
|---|---|---|---|---|---|
| YHFF | 1 | 4 | 4 | | Linker |
| HTTDPSFLGRYMSAL | 5 | 19 | 15 | 0.376208 | epitope |
| FADDLNQLTGY | 20 | 30 | 11 | 2.947135 | epitope |
| HTDFSSEIIGY | 31 | 41 | 11 | 2.944603 | epitope |
| QLMCQPILL | 42 | 50 | 9 | 1.967348 | epitope |
| AEAELAKNVSL | 51 | 61 | 11 | 0.517577 | epitope |
| ILGTVSWNL | 62 | 70 | 9 | 0.596325 | epitope |
| KRRY | 71 | 74 | 4 | | linker |
| LLSAGIFGA | 75 | 83 | 9 | 0.529516 | epitope |
| ITDVFYKENSY | 84 | 94 | 11 | 2.105188 | epitope |
| KVPTDNYITTY | 95 | 105 | 11 | 1.134409 | epitope |
| ARMA | 106 | 109 | 4 | | linker |
| APKEIIFLEGETL | 110 | 122 | 13 | 1.175399 | epitope |
| FGDDTVIEV | 123 | 131 | 9 | 2.679615 | epitope |
| AIILASFSAST | 132 | 142 | 11 | 0.747335 | epitope |
| RRMA | 143 | 146 | 4 | | linker |
| MVTNNTFTLK | 147 | 156 | 10 | 0.742596 | epitope |
| VPHVGEIPVAYRKVLL | 157 | 172 | 16 | 2.066211 | epitope |
| KTIQPRVEK | 173 | 181 | 9 | 2.922006 | epitope |
| YLFDESGEFKL | 182 | 192 | 11 | 2.269281 | epitope |
| SEVGPEHSLAEY | 193 | 204 | 12 | 1.662412 | epitope |
| YIFFASFYY | 205 | 213 | 9 | 0.210118 | epitope |
| KRNG | 214 | 218 | 4 | | linker |

### Example 14. Immunogenicity Studies of Cor Vac 2.0 Strings (C5-C8) in Animal Models

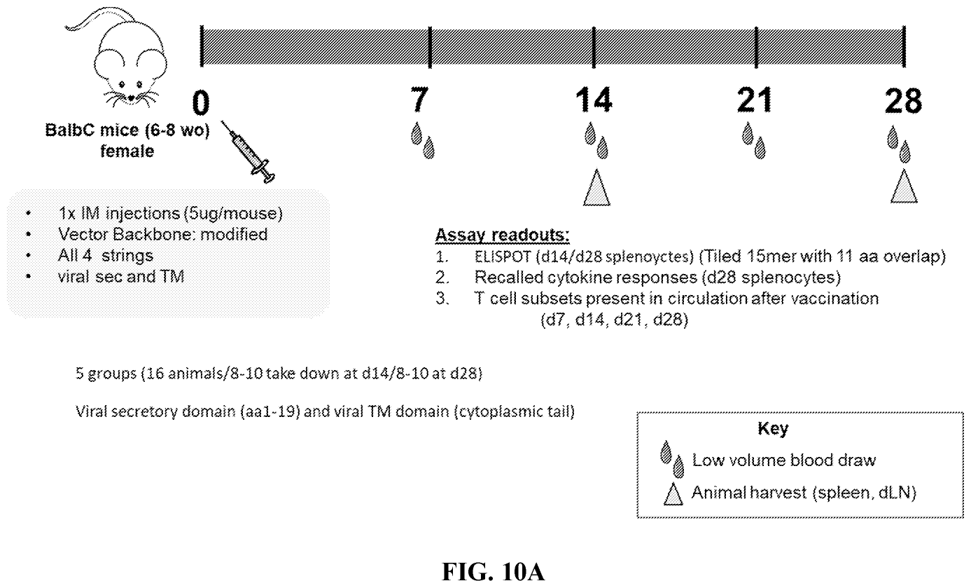

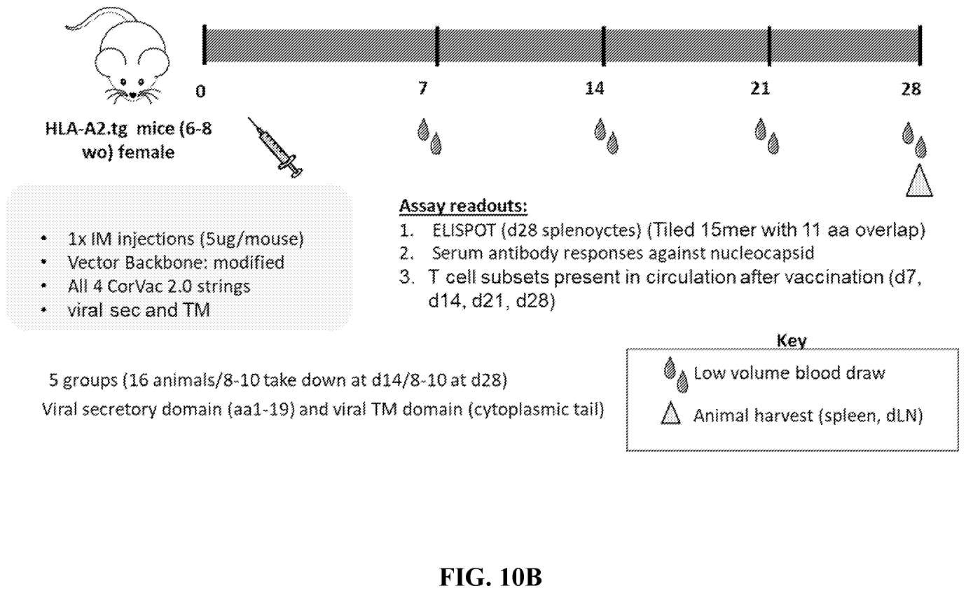

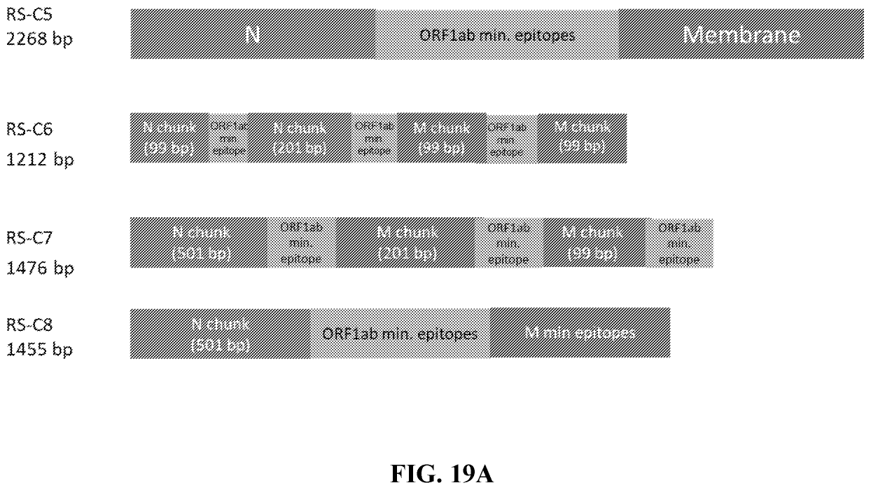

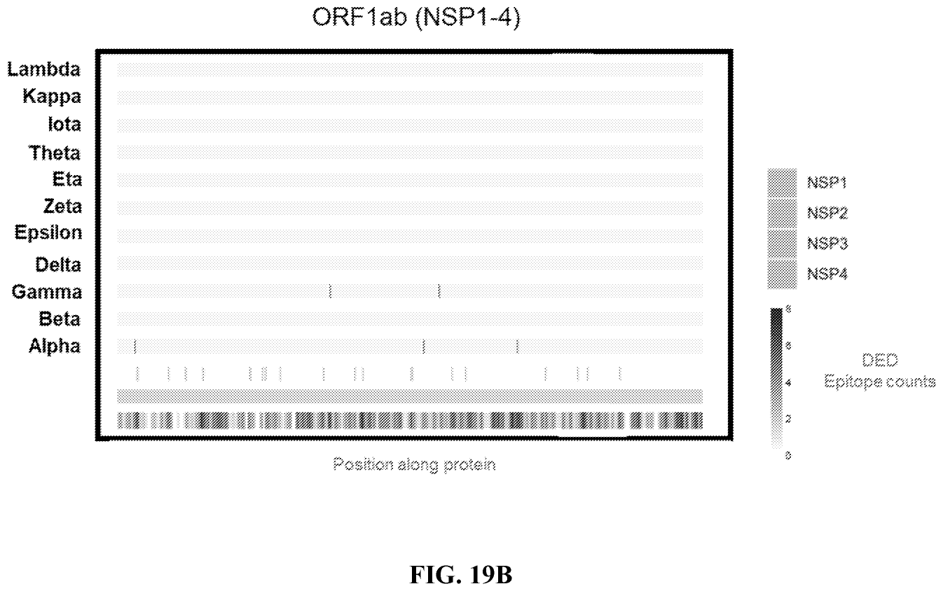

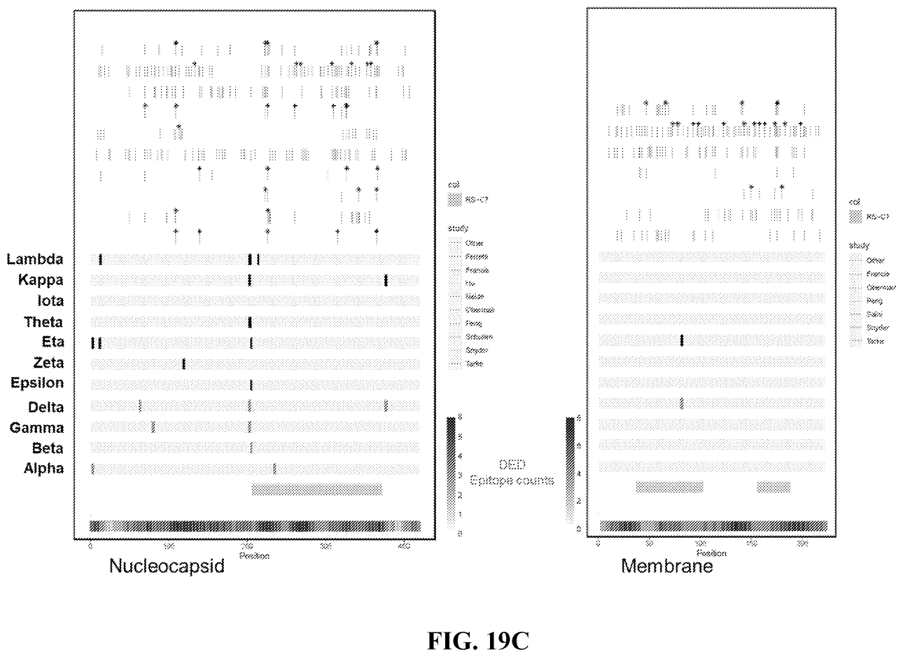

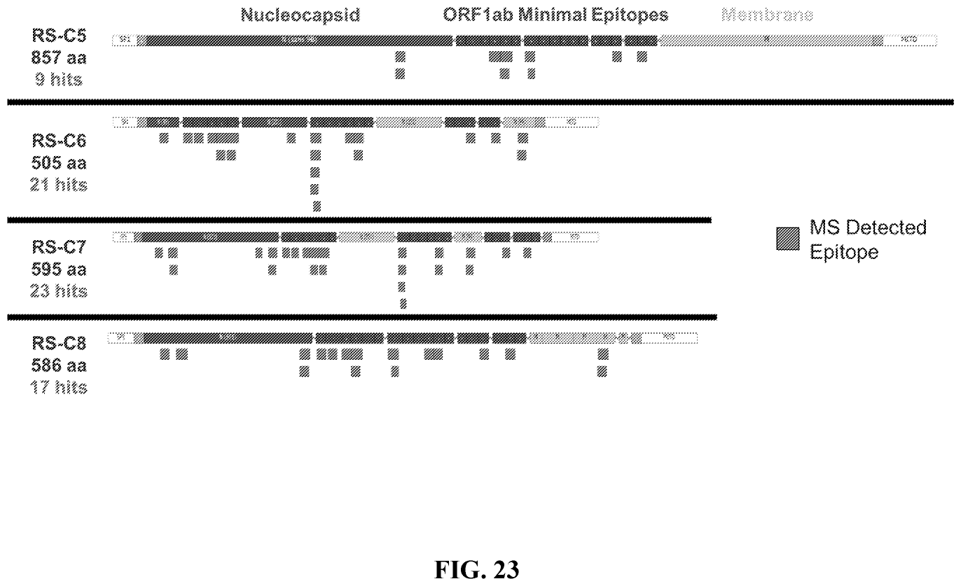

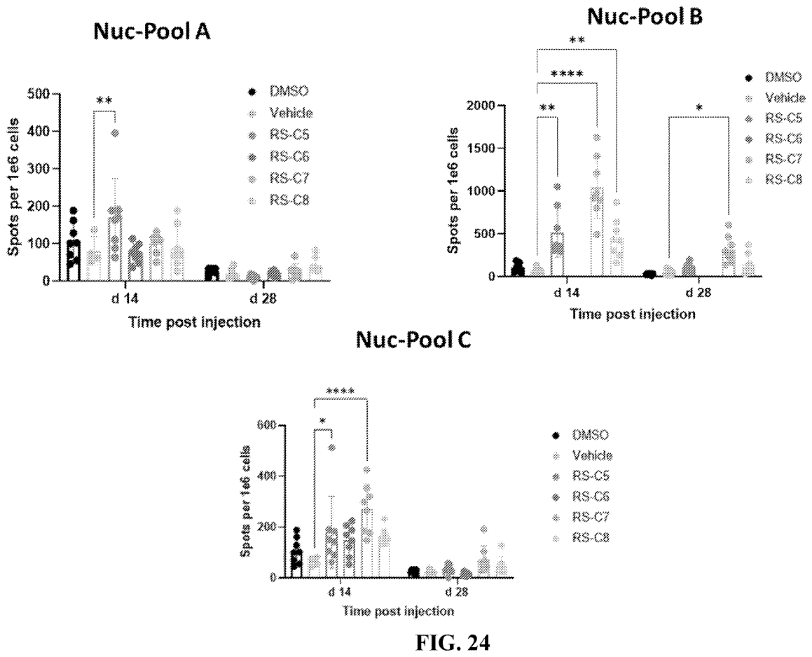

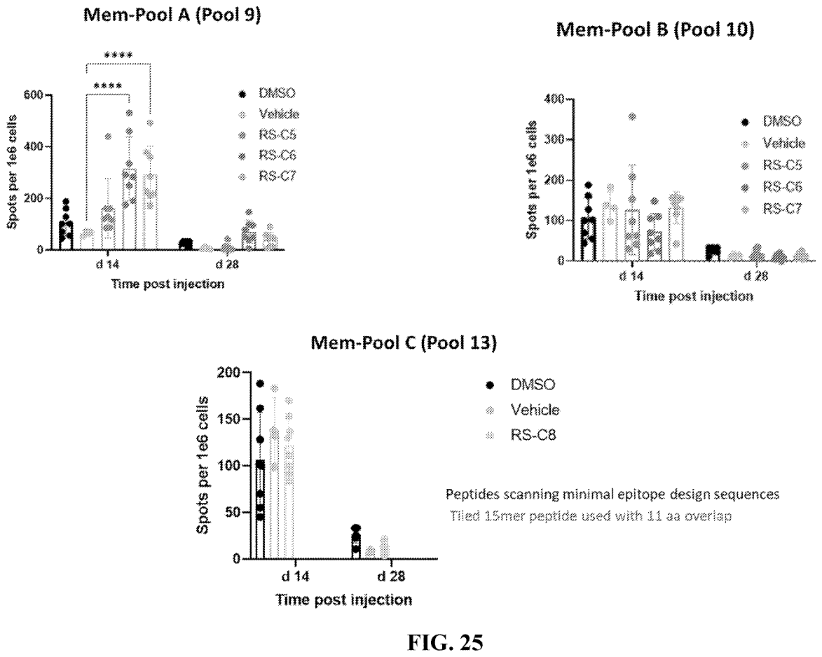

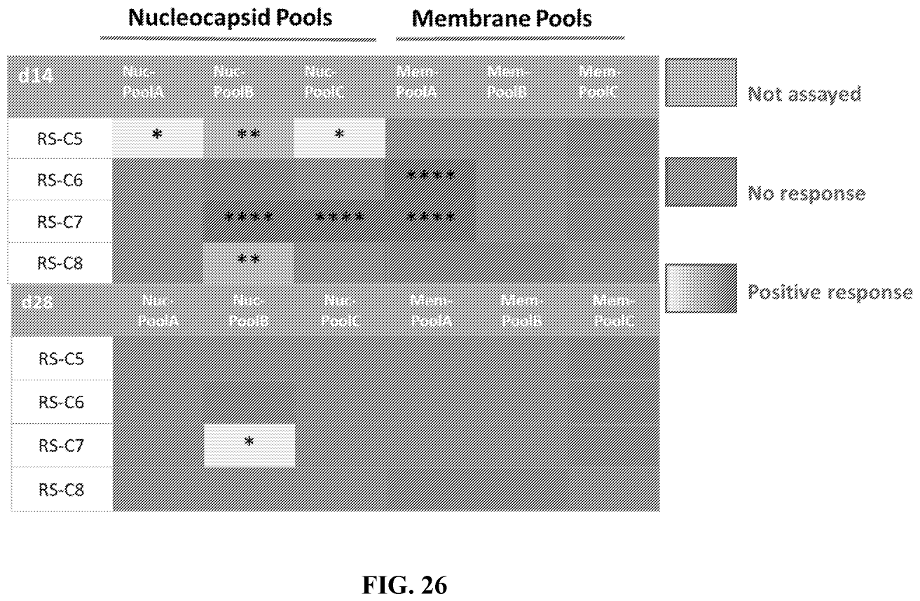

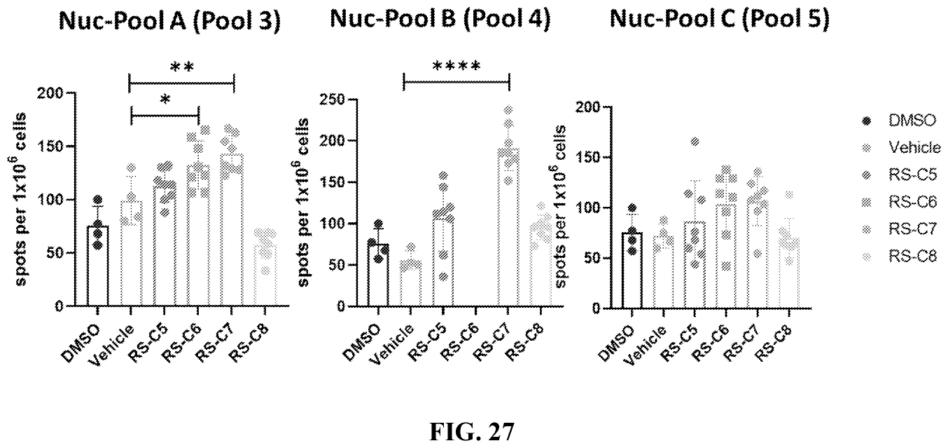

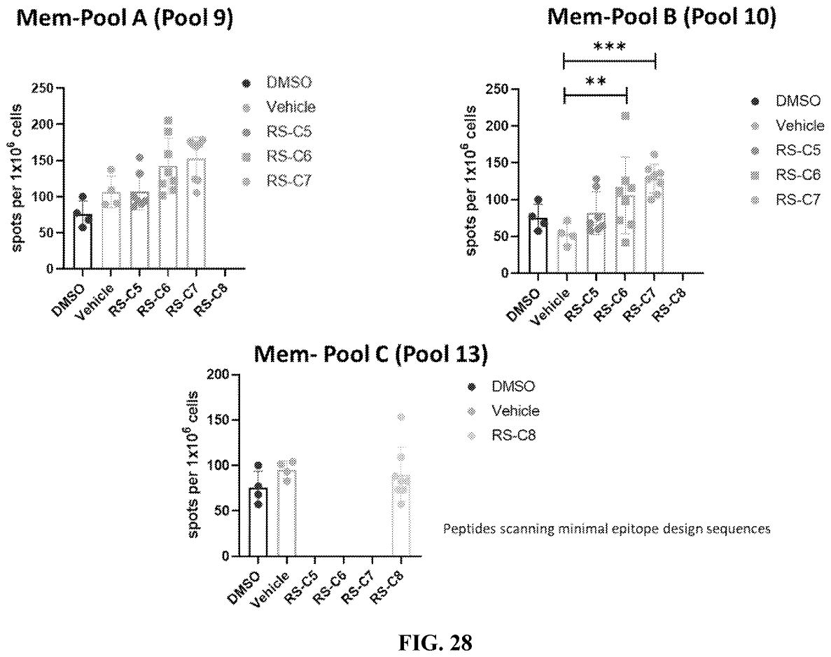

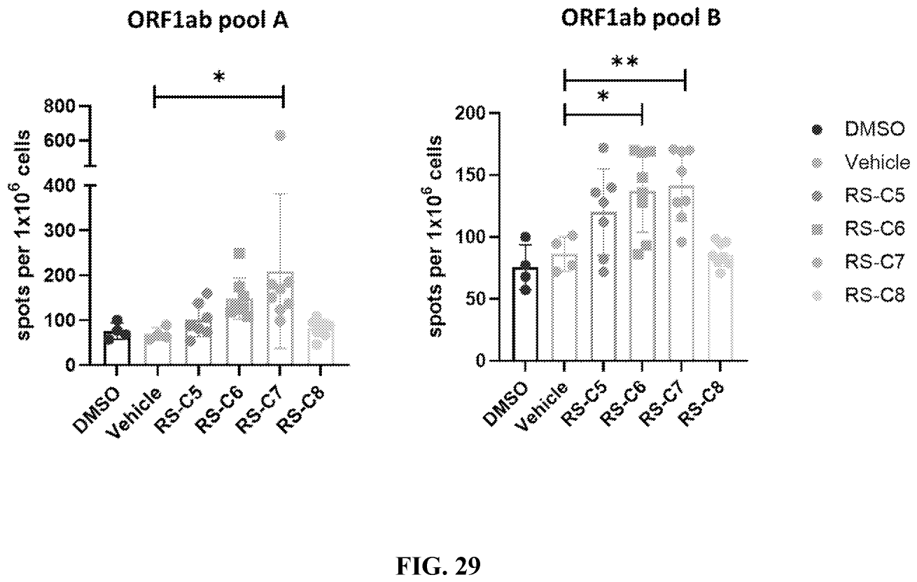

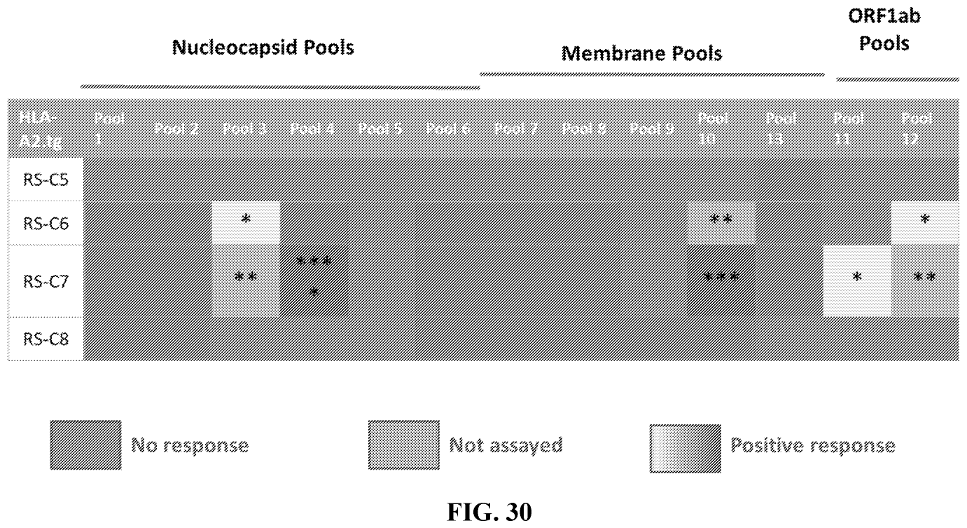

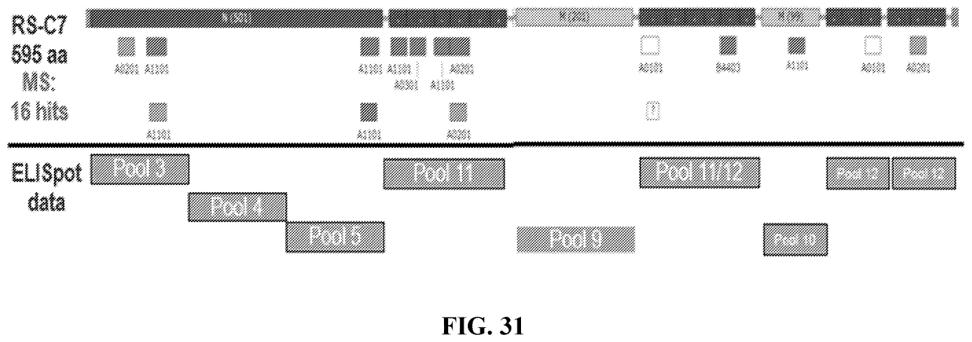

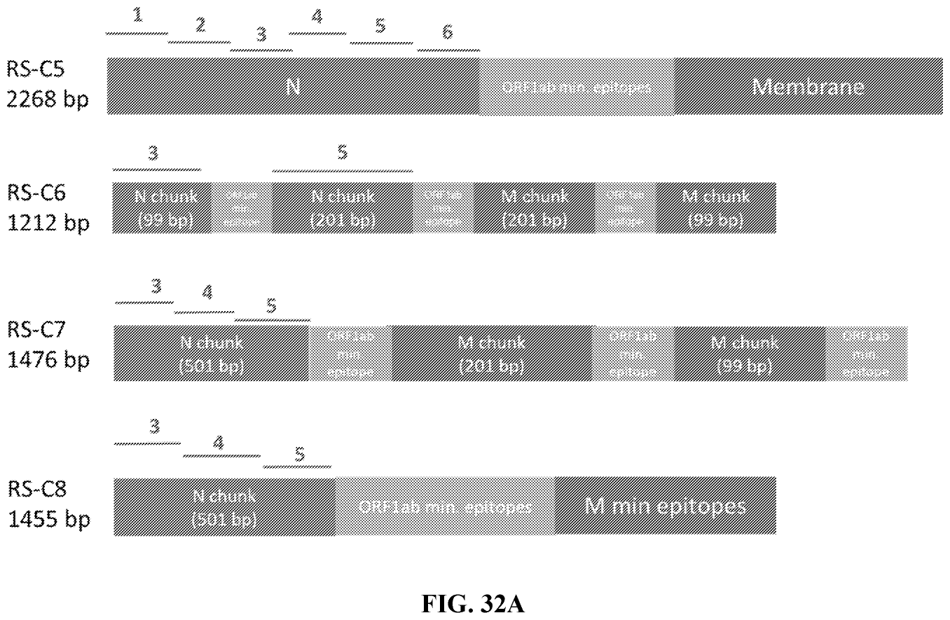

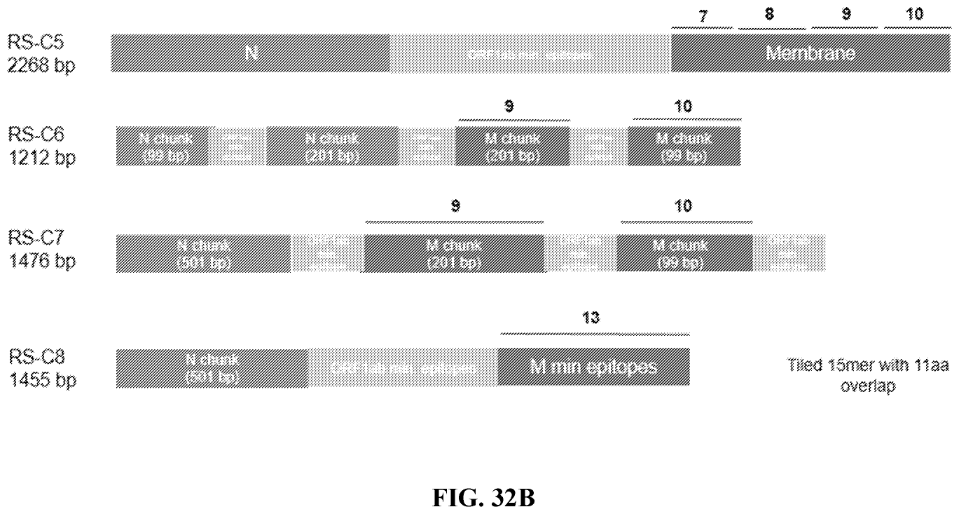

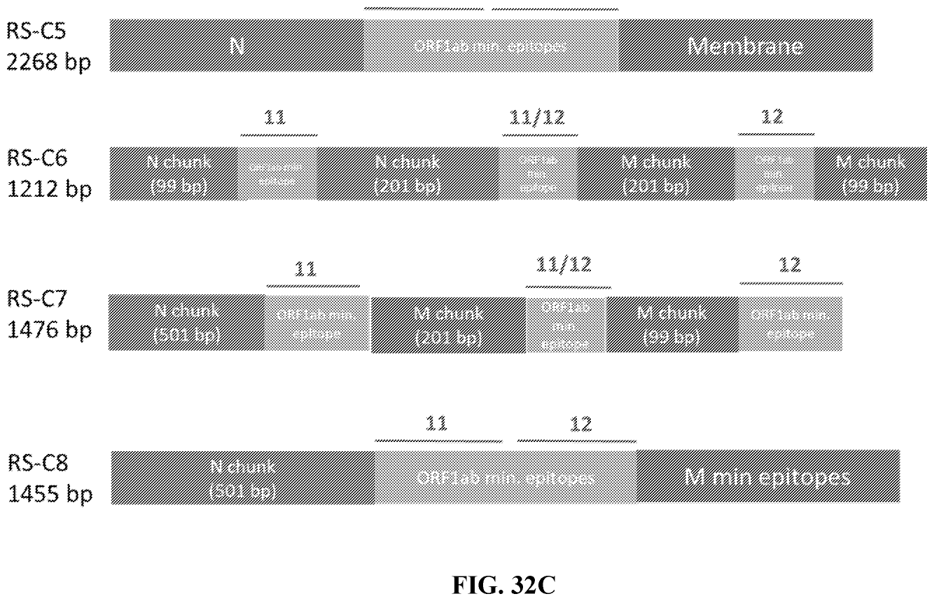

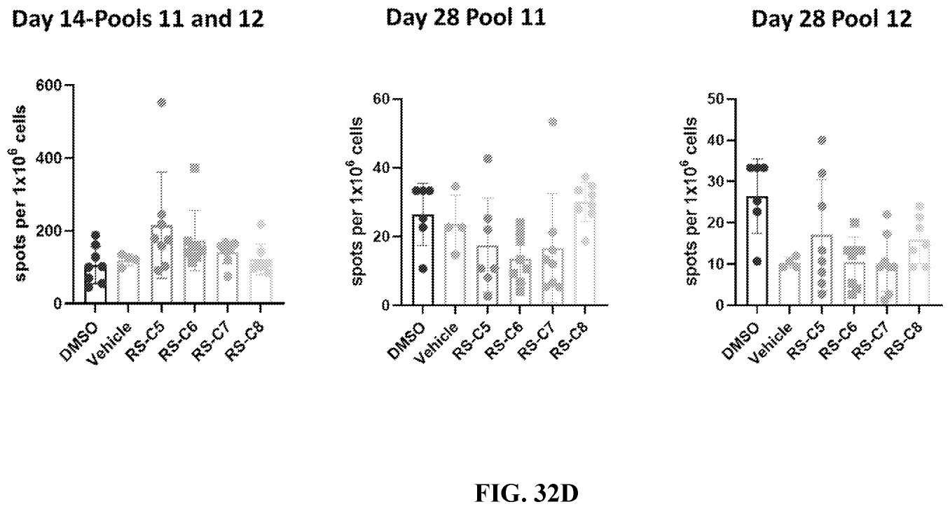

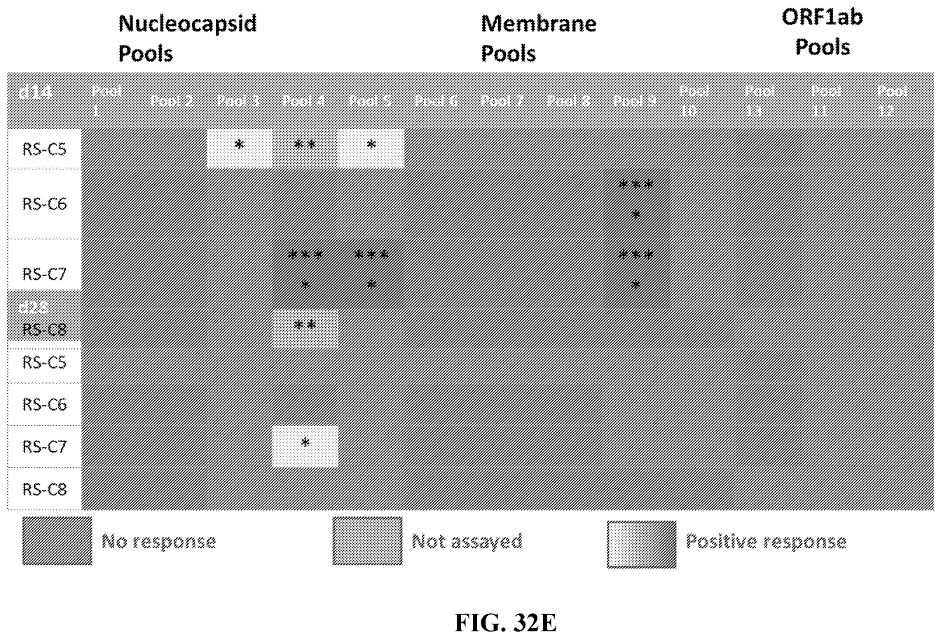

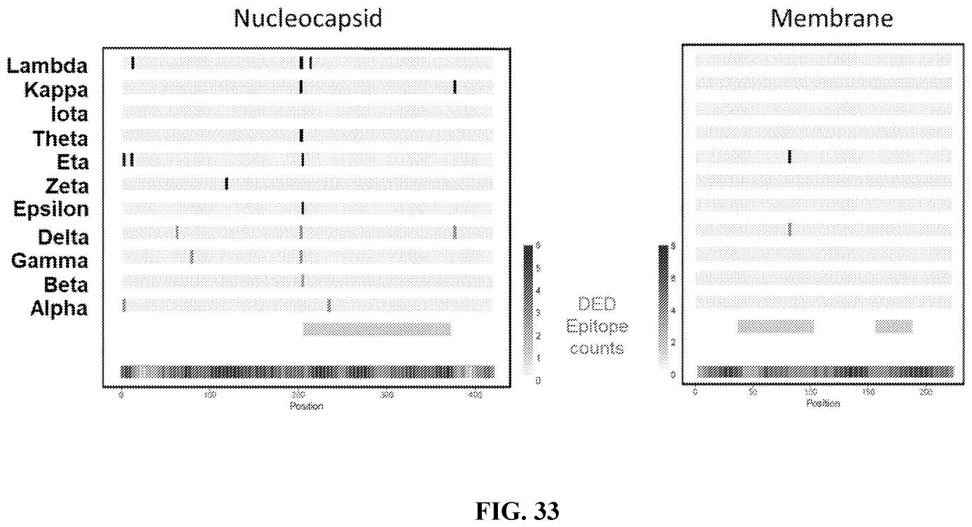

FIG. 19A, FIG. 19B and FIG. 19C further demonstrate the string designs for CorVac 2.0, using sequences encoding 2019 SARS Cov-2 portions of Nucleocapsid (N), membrane and ORF1a/b in four strings (C5-C8) that were further tested for immunogenicity in mice. FIG. 23 demonstrates the MS-detected epitope coverages in RS C5-RS C8 designs. In some studies, sequence encoding tiled 15-mer peptide were used with 11 amino acid overlap in the string designs the Nucleocapsid (Nuc) antigen pool, the membrane (M) antigen pool, or the ORF-1ab pool (FIG. 32A, FIG. 32B, FIG. 32C). One objective of the studies exemplified in FIG. 10A and FIG. 10B was to determine the most immunogenic strings between the C5-C8 CorVac 2.0 strings for further development. The studies exemplified in FIG. 10A and FIG. 10B were used to test the C5-C8 mRNA string vaccines, compare the immunogenicities offered by vaccines targeting viral secretory domains (SEC domains) or the cytoplasmic tail TM domains, and test for immunogenic responses. In the studies exemplified in FIG. 10A and FIG. 10B, each set of 6-8 weeks old female BALB/C mice or HLA-A2 transgenic mice are administered intramuscular injections of one of the four mRNA string construct (5 microgram/mouse), and blood samples are collected at 7, 14, 21 and 28 days post injection. Animals are euthanized on day 28 (FIG. 10B) or on day 14 and on day 28 (FIG. 10A) and organs are harvested. FIG. 24 and FIG. 25 show that all 4 strings elicited T cell response, i.e., CorVac 2.0 encoded nucleocapsid epitope pools A, B and C and membrane pools A, B and C (tiled 15mer peptide with 11 aa overlap) elicit T cell response. Similarly, T cell response was elicited in HLA-A2 transgenic mice from the nucleocapsid and membrane regions using the CorVac 2.0 strings (FIG. 27 and FIG. 28). T cell responses to ORF 1ab epitopes were present in the HLA-A2 transgenic mice (FIG. 29). In contrast, as shown in FIG. 32D, ORF1ab epitopes do not elicit T cell response in BALB/c mice which do not express HLA-A2. From these studies overall, specifically summarized in FIG. 26, FIG. 30, FIG. 32E it is evident that the RS-C7 string elicits the most T cell response from nucleocapsid, membrane and ORF1ab regions. FIG. 31 shows the distribution and coverage of the epitope pools in the C7 string. Additionally, the C7 string overlaps with few or no mutations in the variants of SARS-CoV-2 strain that are of concern (FIG. 33).

### Example 15. Correlation of RECON Prediction with Validation from Independent Studies

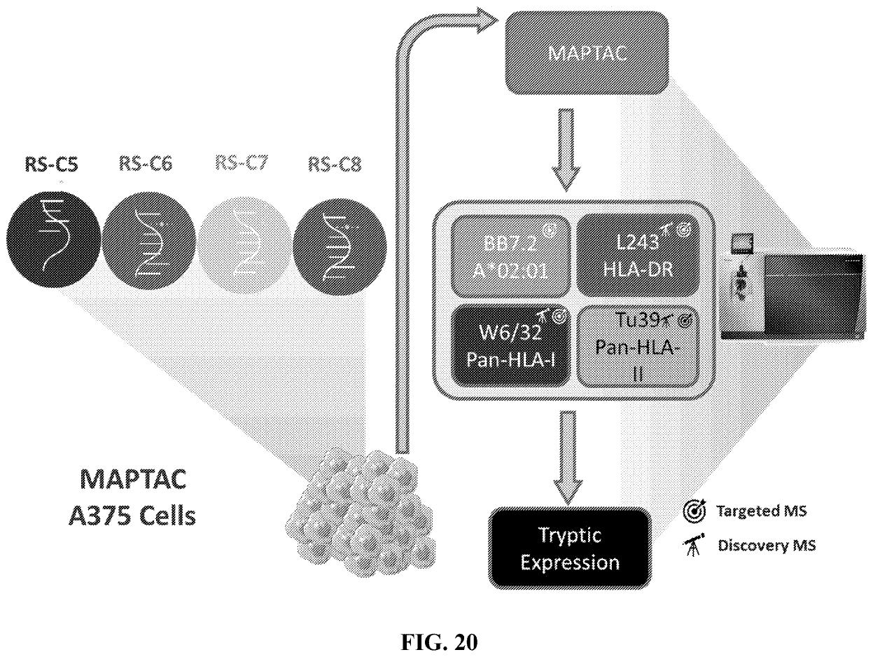

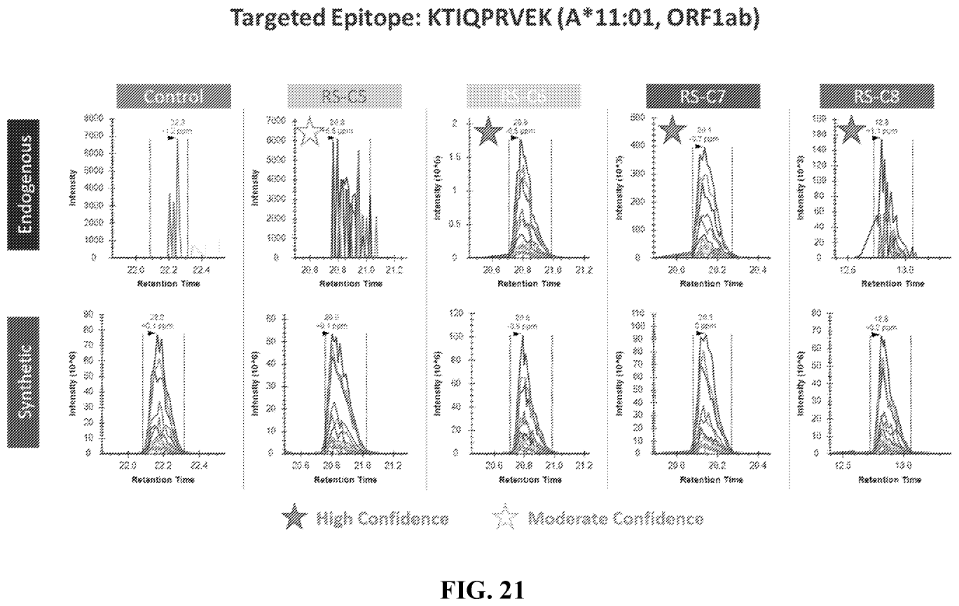

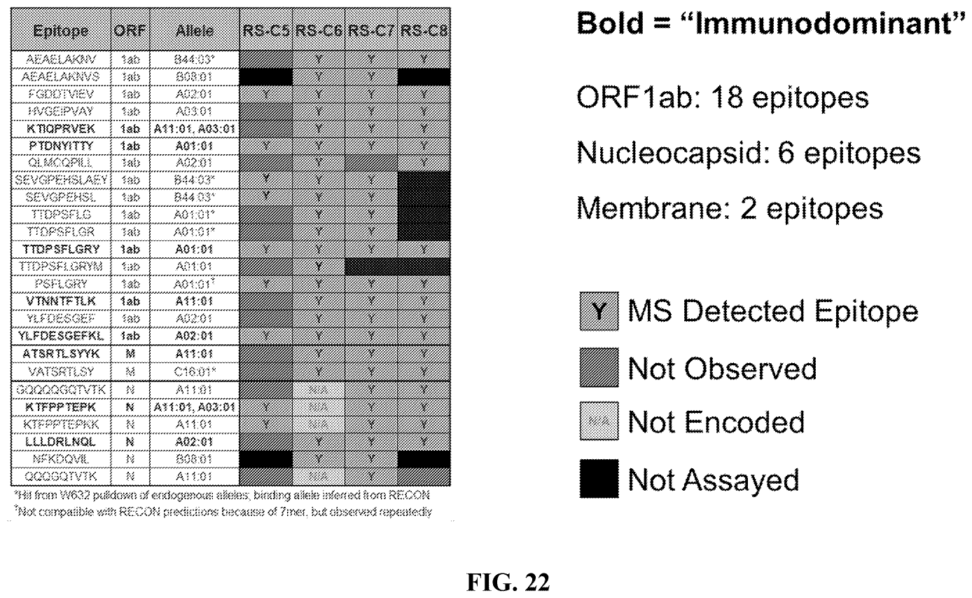

A large body of data derived from T cell immunogenicity studies and mass spectrometry studies is currently available worldwide that strongly correlates with and validates the epitope predictions first reported in this study and provided in epitope sequence of Table 1A, Table 1B, Table 1C, Table 2Ai, Table 2Aii, Table 2B, Table 9, Table 10, Table 11, Table 12, Table 14A, Table 14B, Table 14C, Table 15 or Table 16. Such validation and strong correlation in studies performed worldwide renders high confidence on the reliability of the prediction algorithm. RECON predicted 4794 unique peptide-MHC pairs with ≤1% rank. Table 14A and Table 14B exemplify the MS observed validations of the epitopes. FIG. 20 illustrates the workflow for choice of epitopes for string designs, using the presentation prediction by RECON software with mass spectrometric (MS) validation. FIG. 21 demonstrates high confidence of the prediction using an exemplary targeted ORF1ab epitope KTIQPRVEK that binds to A*11:01, encoded in all 4 strings tested. Also, as demonstrated in FIG. 22, twenty five unique CorVac 2.0 epitopes have been identified using MS, of which, there were 17 ORF1ab epitopes, 6 nucleocapsid epitopes, and 2 membrane epitopes.

TABLE 14A
Exemplary epitopes of BNT- Spike Vaccine Construct

| | | | Literature Mentions | |
|---|---|---|---|---|
| Epitope | Allele | Confidence | Exact | Not Exact |
| KIADYNYKL | A*02:01 | High | Yes | Yes |
| YLQPRTFLL | A*02:01 | High | Yes | Yes |
| TLDSKTQSL | A*02:01 | High | Yes | Yes |
| ELLHAPATV | A*02:01 | High | No | Yes |
| RLQSLQTYV | A*02:01 | High | Yes | Yes |
| RLNEVAKNL | A*02:01 | Moderate | Yes | No |
| RLITGRLQSL | A*02:01 | Moderate | Yes | Yes |
| KCYGVSPTKL | A*03:01 | High | No | Yes |
| TGSNVFQTR | A*68:01 | High | Yes | Yes |
| FPQSAPHGVVF | B*35:01 | High | Yes | Yes |
| QPTESIVRF | B*35:01 | High | Yes | Yes |
| VASQSIIAY | B*35:01 | High | Yes | Yes |
| TSNQVAVLY | B*35:01 | High | Yes | Yes |
| TEVPVAIHADQL | B*40:01 | High | No | No |
| VEKGIYQT | B*41:02 | High | No | Yes |
| VRDPQTLEI | C*05:01 | High | No | No |

TABLE 14B
Exemplary Multi-protein Epitope String Constructs

| | | | | Literature Mentions | |
|---|---|---|---|---|---|
| Epitope | Allele | Confidence | ORF | Exact | Not Exact |
| YLFDESGEFKL | A*02:01 | High | 1ab | Yes | Yes |
| YLFDESGEF | A*02:01 | High | 1ab | No | Yes |
| FGDDTVIEV | A*02:01 | High | 1ab | Yes | Yes |
| LLLDRLNQL | A*02:01 | High | N | Yes | Yes |
| QLMCQPILL | A*02:01 | Moderate | 1ab | Yes | Yes |
| TTDPSFLGRY | A*01:01 | High | 1ab | Yes | Yes |
| PTDNYITTY | A*01:01 | High | 1ab | Yes | Yes |
| PSFLGRY | N/A | High | 1ab | No | Yes |
| AEAELAKNV | B*44:03 | High | 1ab | Yes | Yes |
| VATSRTLSY | C*16:01 | High | M | Yes | Yes |
| KTIQPRVEK | A11:01 | High | 1ab | Yes | Yes |

TABLE 14C
This table lists the MS identified epitopes
derived from CorVac 2.0 C5-C8 string designs.

| Epitope | ORF | Allele |
|---|---|---|
| AEAELAKNV | 1ab | B44:03 |
| AEAELAKNVS | 1ab | B08:01 |
| FGDDTVIEV | 1ab | A02:01 |
| HVGEIPVAY | 1ab | A03:01 |
| KTIQPRVEK | 1ab | A11:01, A03:01 |
| PTDNYITTY | 1ab | A01:01 |
| QLMCQPILL | 1ab | A02:01 |
| SEVGPEHSLAEY | 1ab | B44:03 |
| SEVGPEHSL | 1ab | B44:03 |
| TTDPSFLG | 1ab | A01:01 |
| TTDPSFLGR | 1ab | A01:01 |
| TTDPSFLGRY | 1ab | A01:01 |
| TTDPSFLGRYM | 1ab | A01:01 |
| PSFLGRY | 1ab | A01:01 |
| VTNNTFTLK | 1ab | A11:01 |
| YLFDESGEF | 1ab | A02:01 |
| YLFDESGEFKL | 1ab | A02:01 |
| ATSRTLSYYK | M | A11:01 |
| VATSRTLSY | M | C16:01 |
| GQQQQGQTVTK | N | A11:01 |
| KTFPPTEPK | N | A11:01, A03:01 |
| KTFPPTEPKK | N | A11:01 |
| LLLDRLNQL | N | A02:01 |
| NFKDQVIL | N | B08:01 |
| QQQGQTVTK | N | A11:01 |

The above data represent 19 studies, covering 1180 class I epitopes, including 881 class I unique epitope sequences. As many as 756 (86%) have exact match to RECON predictions. 872 (98%) sequences have either a superstring or a substring in RECON predictions. These studies indicate high degree of correlation of epitope prediction by RECON with actual T cell immunogenicity observed data.

### Example 16. Sequence Variability of SARS CoV-2

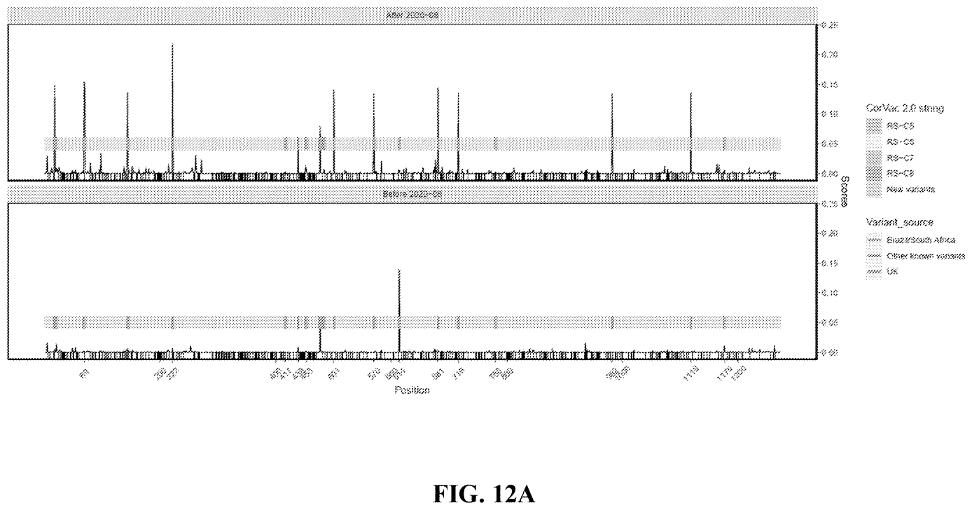

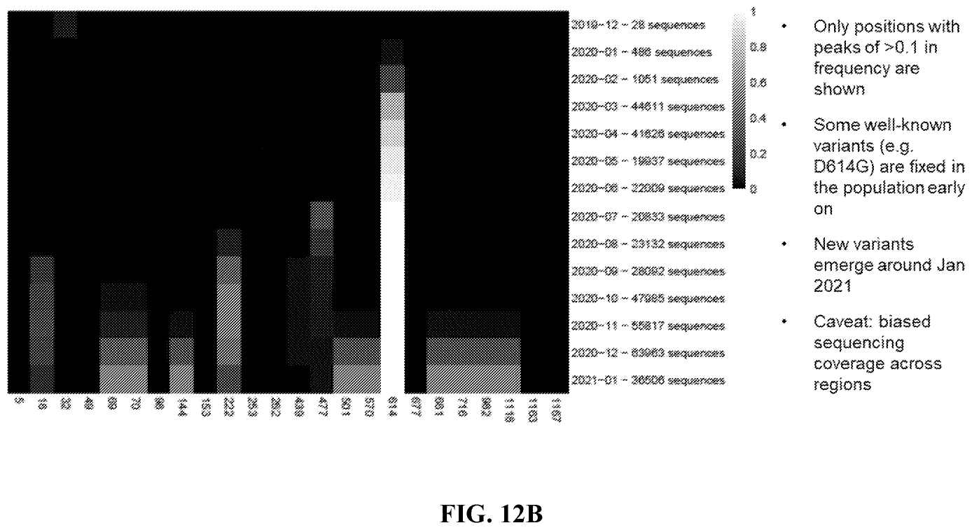

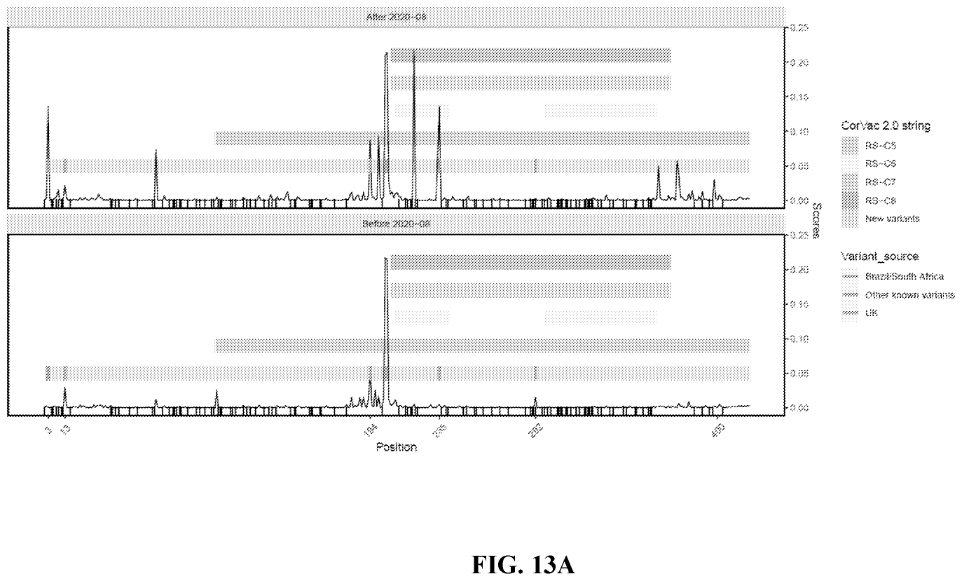

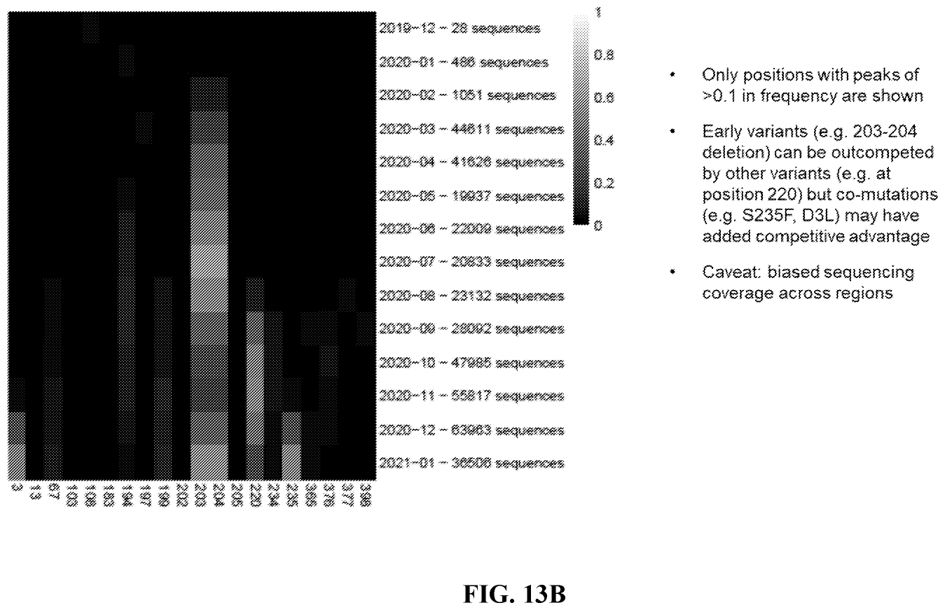

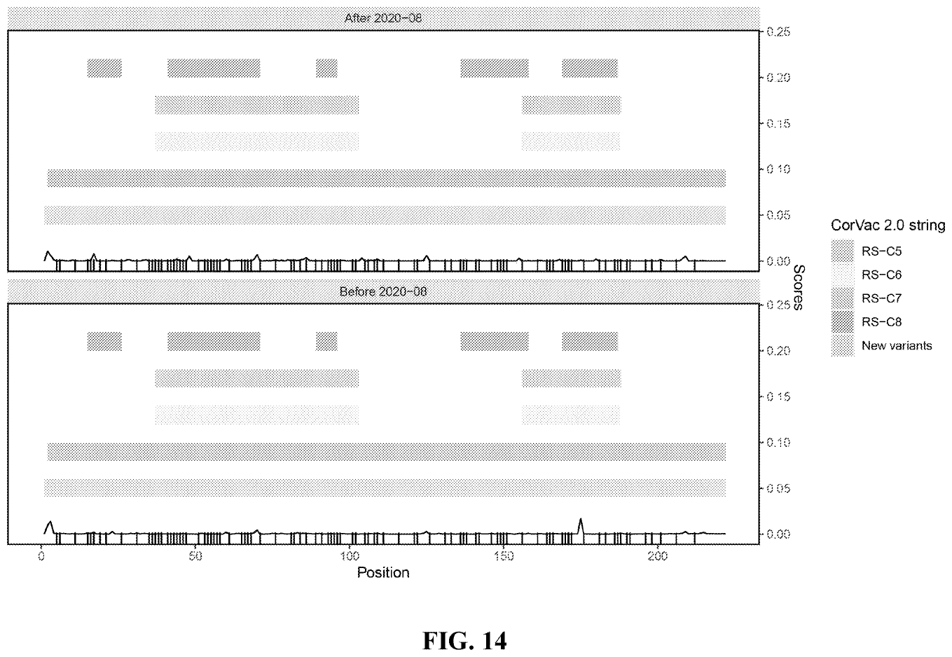

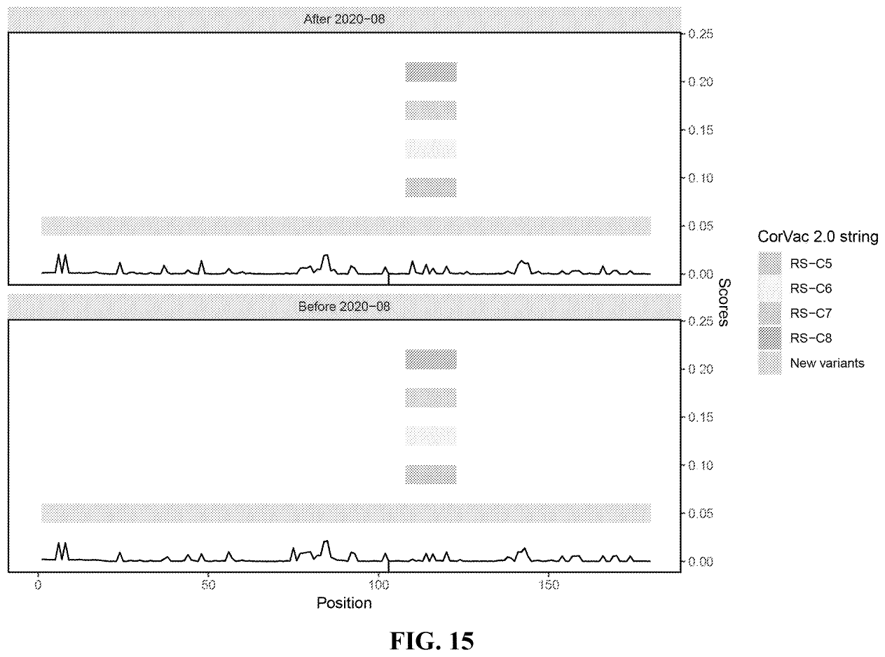

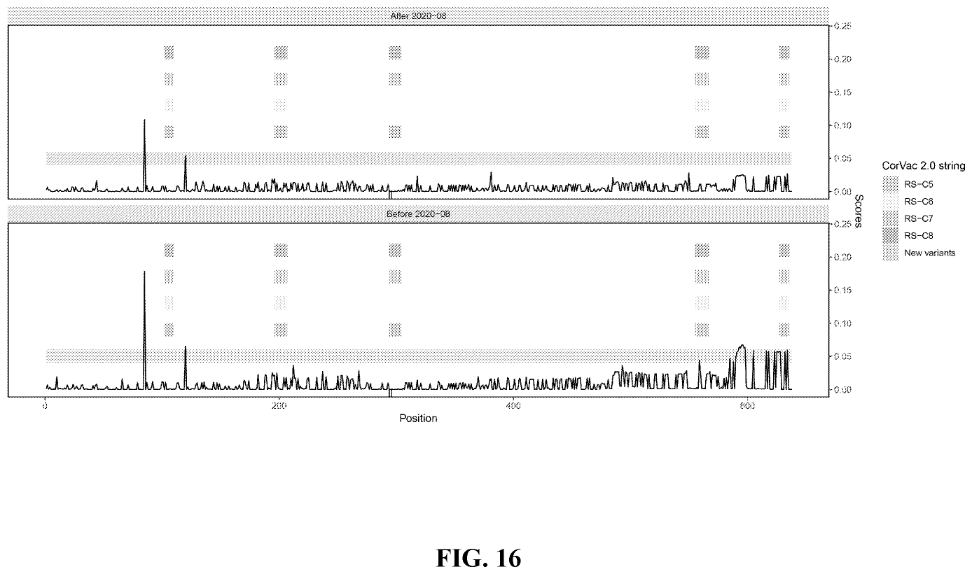

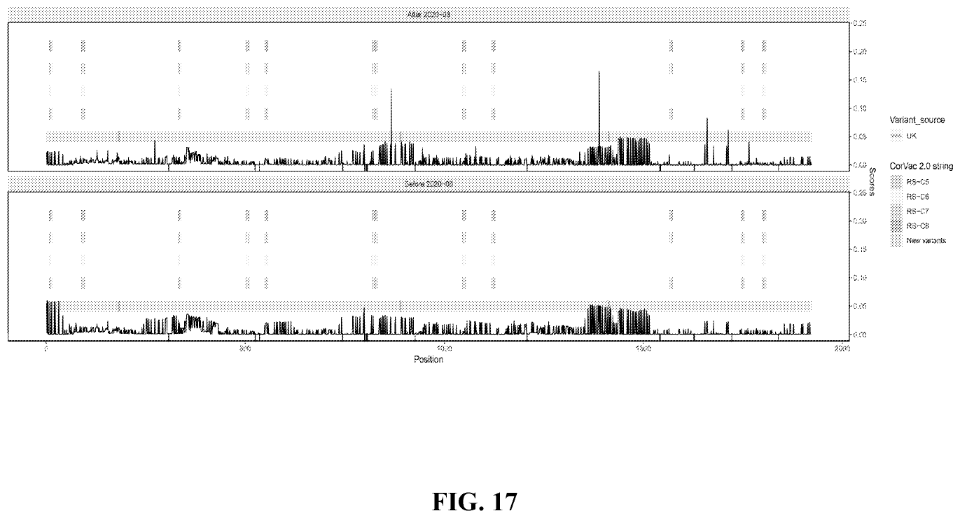

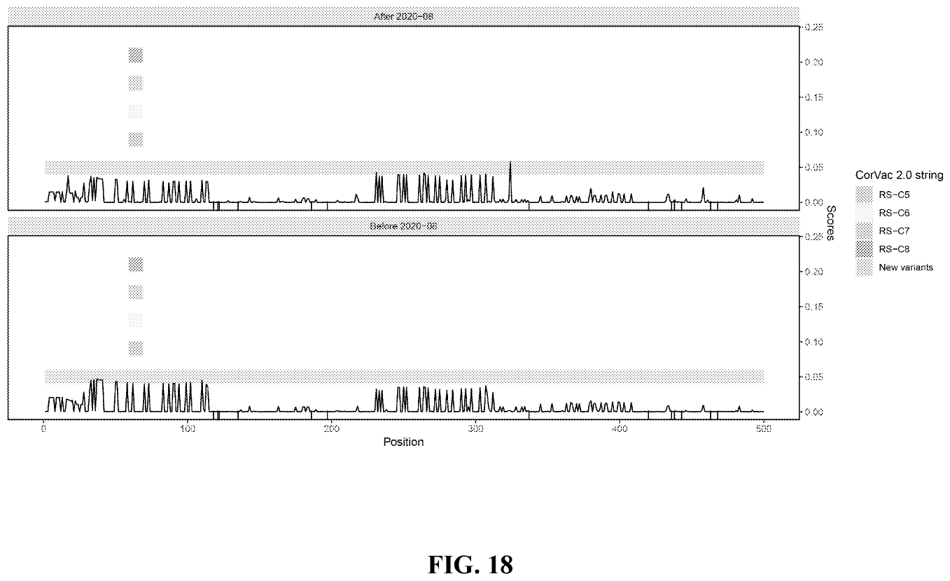

In this example, the sequence variability of the SARS CoV-2 is shown, in light of protection that the string vaccines can offer. FIG. 12A, FIG. 12B, FIG. 13A, FIG. 13B, FIG. 14, FIG. 15, FIG. 16, FIG. 17 and FIG. 18 demonstrates detection/identification of variant or mutated amino acid positions along the SARS CoV-2 genome. This was in turn used to assess the sensitivity of string vaccines to variants. The figures highlight the regions covered by the epitopes covered by the strings. As shown at least in these figures, and also combined with information on the string sequences provided elsewhere in the document, multiple epitopes from multiple different viral proteins are included in each string. That is, if one epitope covers a mutant sequence, the other epitopes are at least spared to offer immune response against a viral strain. This design ensures that the not only a wider and more robust immunogenic response can be triggered by the vaccine, it is also important to note that different variants and mutants of the virus are covered by a string vaccine. It follows that the design of the constructs offers a good mechanism of avoiding viral escape variants, as is demonstrated in the analysis in FIG. 12A, FIG. 12B, FIG. 13A, FIG. 13B, FIG. 14, FIG. 15, FIG. 16, FIG. 17 and FIG. 18. This provides support that the string vaccines can confer protective immunity that is not variant-dependent.

### Example 17. Predictions Using HLA-Peptide Presentation Prediction Algorithm

An updated run in the machine-learning HLA-peptide presentation prediction algorithm RECON predicted newer epitopes that were predicted with high score, (that is, a score that represents high likelihood that the epitope peptide would actually be presented by the HLA allele). These epitope-HLA pairs are listed below in Table 16. For each pair, the epitope peptide sequence in the left column (column 1 or 3) is predicted to be presented by the HLA in the column immediately to its right (column 2 or column 4 respectively) in the same row.

### Example 18. Immunogenicity Studies of Cor Vac 2.0 C-7 String with BNT162b2 in BALB/C Mice

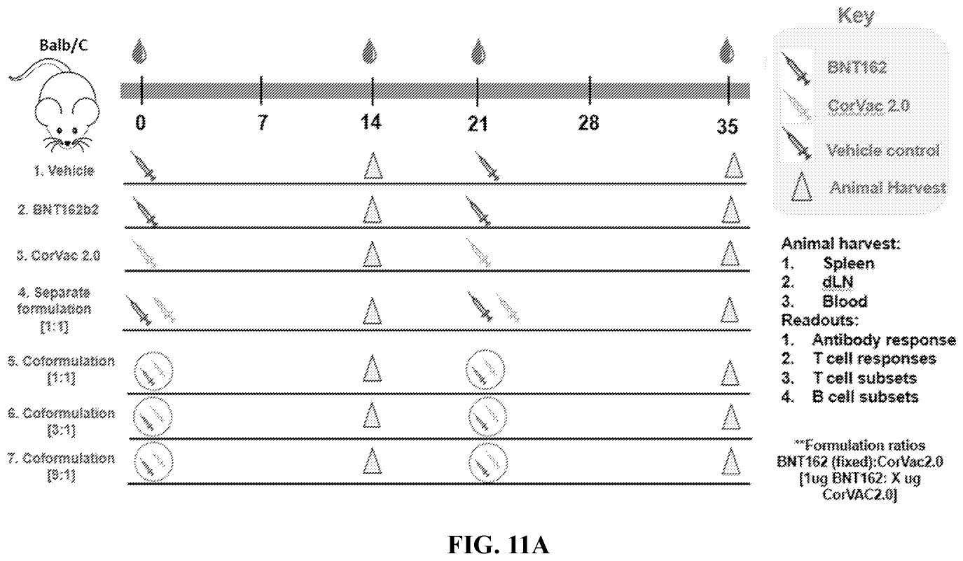

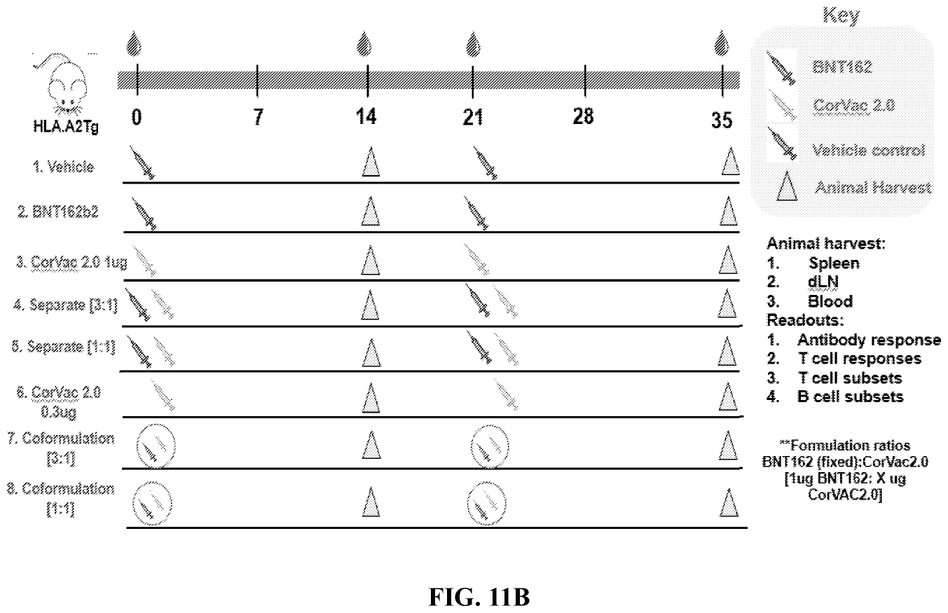

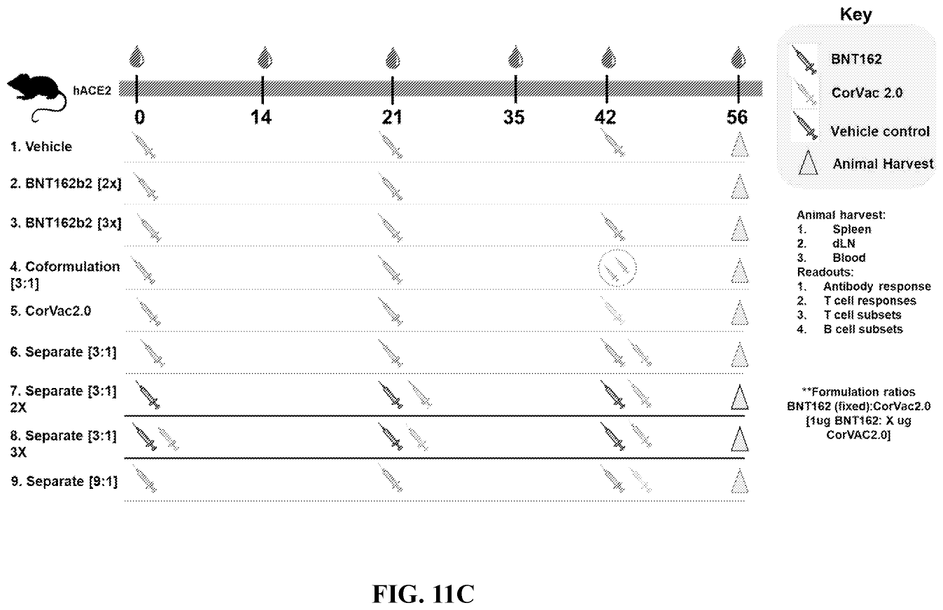

In one study design as exemplified in FIG. 11A, different vaccine strategies are compared in BALB/C mice to examine the immunogenicity of CorVac 2.0 strings with BNT162b2, to test the impact of CorVac 2.0 on anti-Spike antibodies induced by BNT162b2, test CorVac 2.0 T cell responses after prime and boost (2 shots), compare combinations of BNT162b2 and CorVac 2.0 either through separate formulation (different LNPs) or co-formulation (same LNP), and to test varying ratios of BNT162b2: CorVac 2.0, keeping BNT162b2 dosage fixed to not reduce Ab titers. As exemplified in FIG. 11A, the different vaccine strategies in BALB/C mice for this study include: (a) the spike mRNA vaccine alone, (b) the string vaccine alone, (c) a first formulation of the spike mRNA vaccine and a separate second formulation of the string vaccine administered on the same day, (d) co-formulation of the spike vaccine and the string vaccine in a first ratio (e.g., 1:1); (e) a co-formulation of the spike vaccine and the string vaccine in a second ratio (e.g., 3:1); and (f) a co-formulation of the spike vaccine and the string vaccine in a third ratio (e.g., 9:1). In this study, about half of the mice of each set are sacrificed on day 14, and the rest on day 35. Samples are collected at day 0, 14, 21 and 35 after the first injection. ELISPOT is performed, cytokine and chemokine generation is analyzed, and T cell subsets are tested for antiviral cytokine responses, surface marker expression, and expression of lineage markers is analyzed by flow cytometry. Antigen specific responsiveness of T cells is also measured in vitro. Tissue samples are also tested for presence of spike antibody, T cell responses and T cell subsets in spleen, draining lymph nodes and blood.

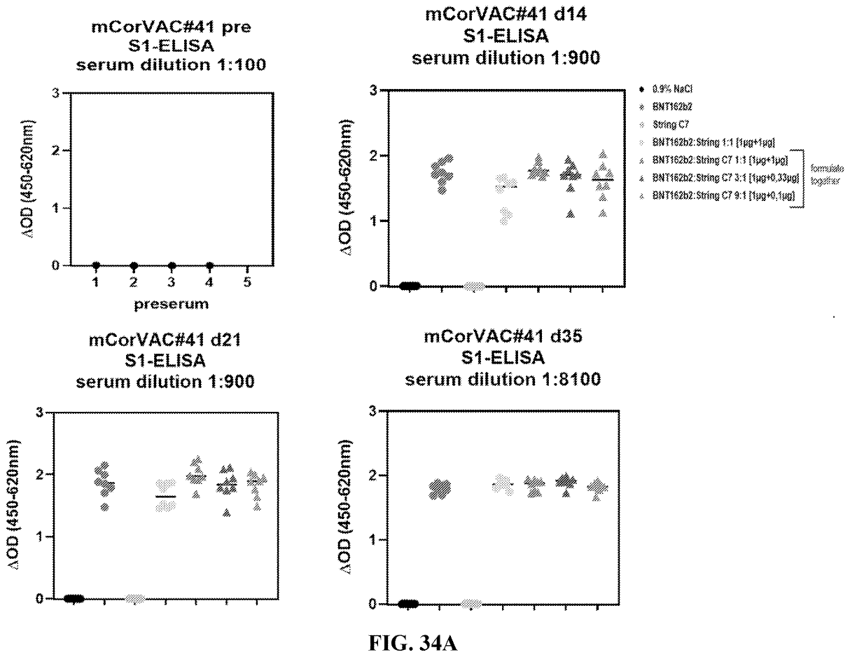

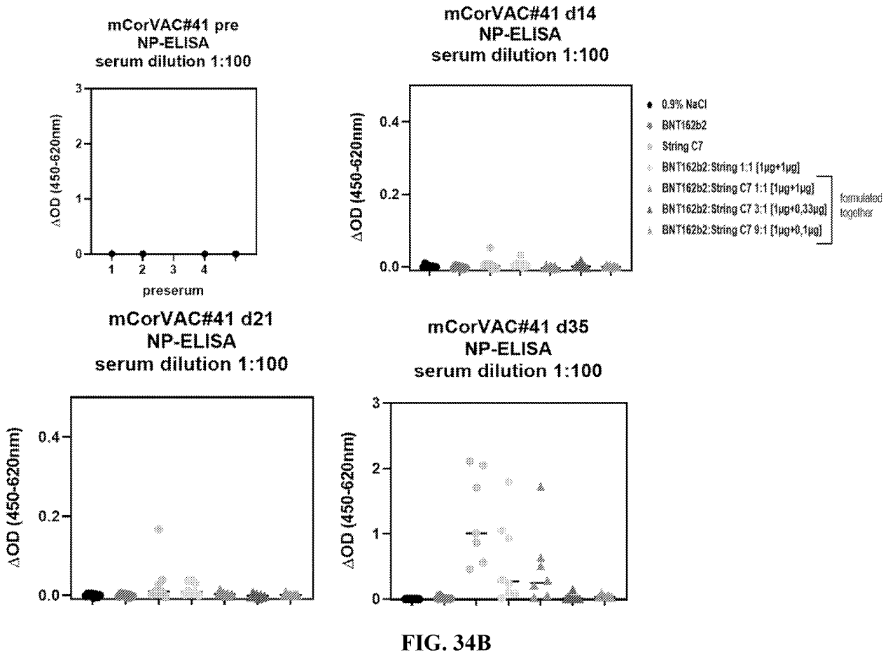

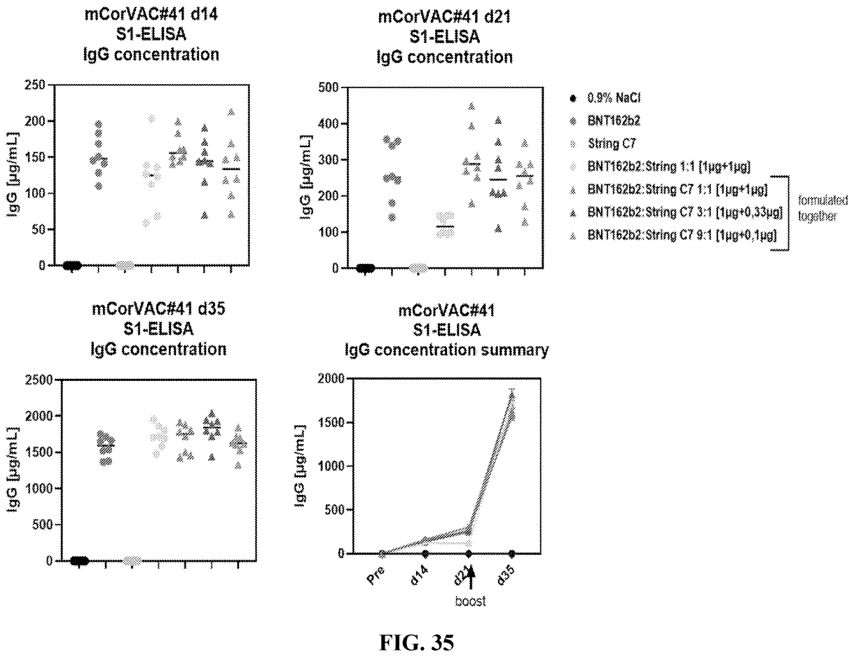

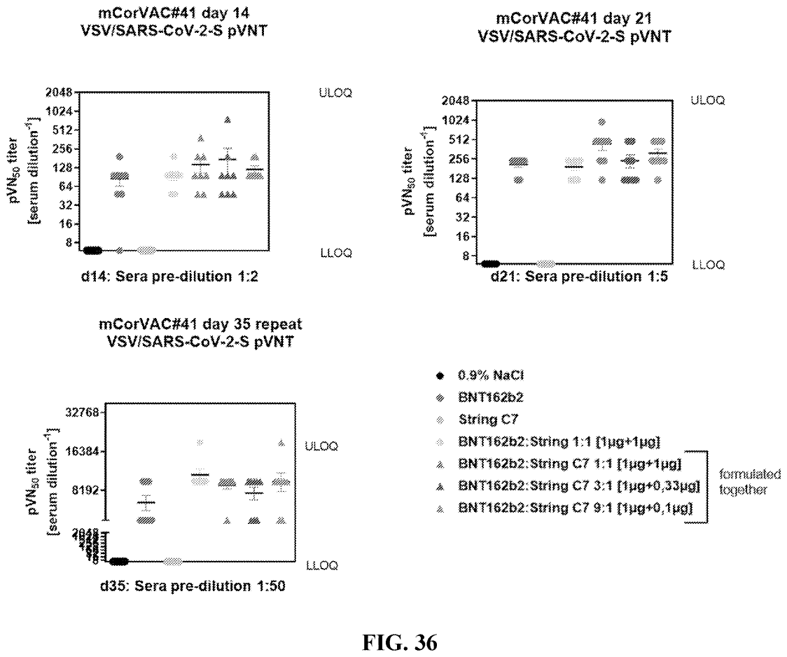

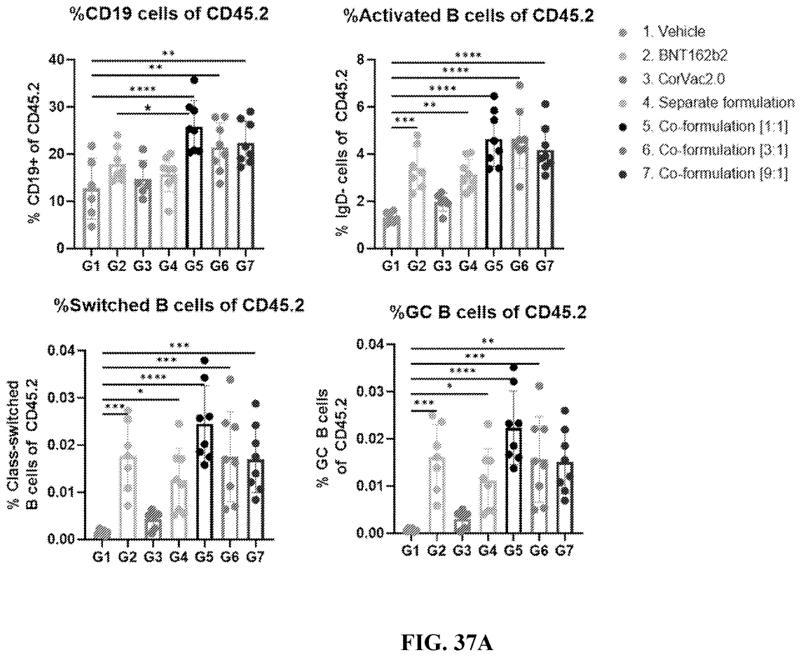

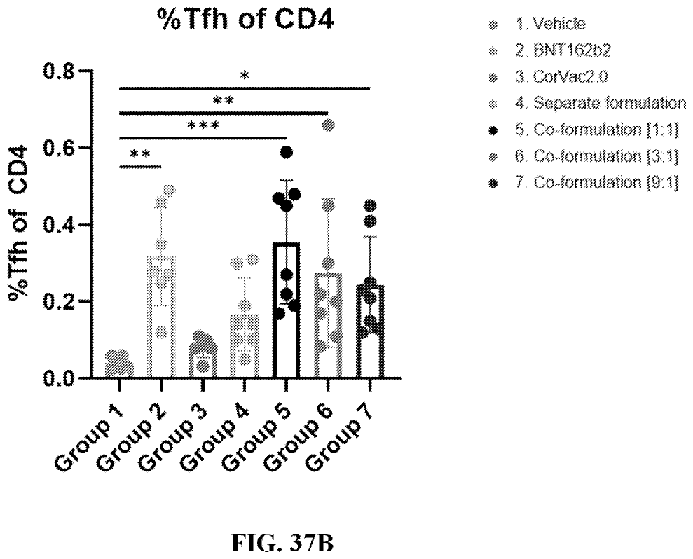

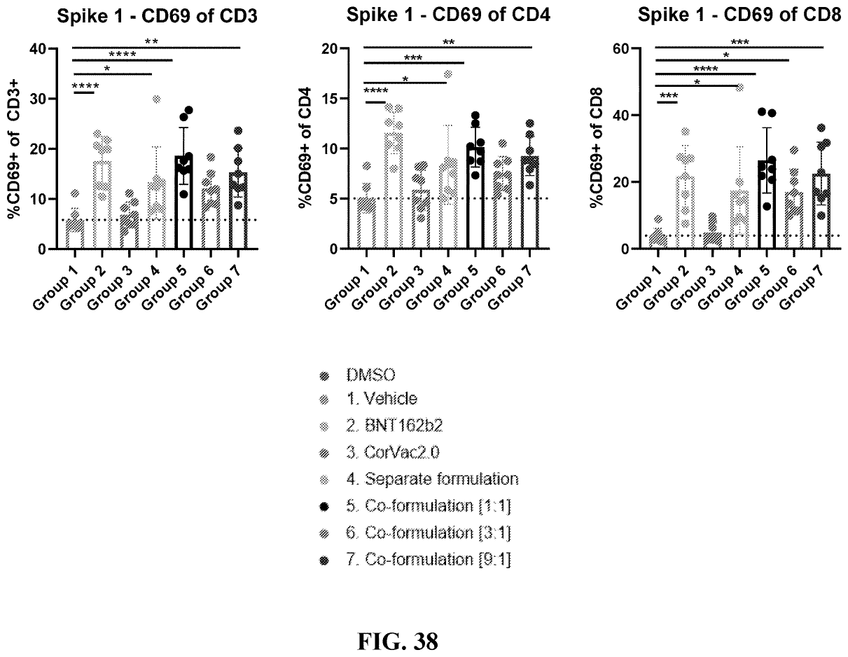

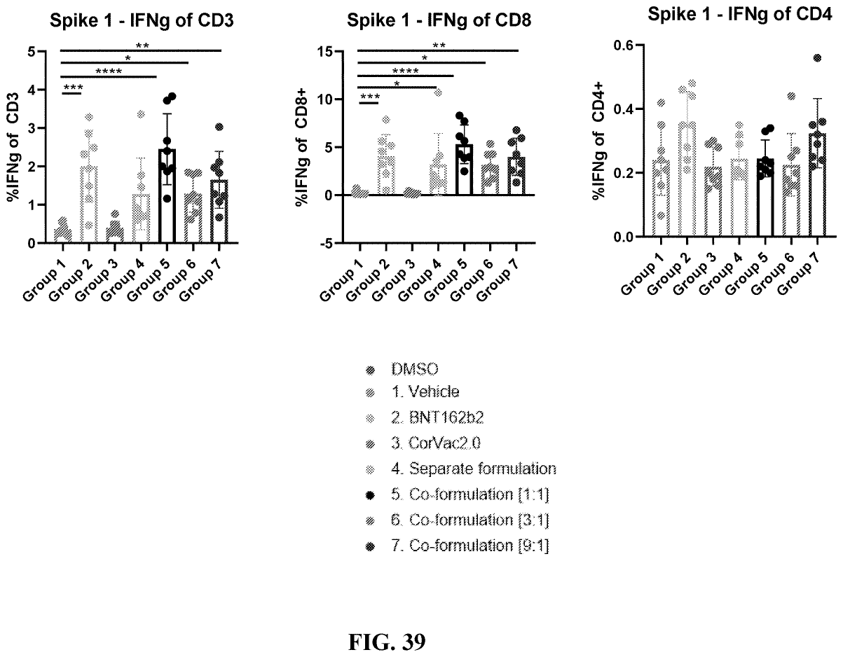

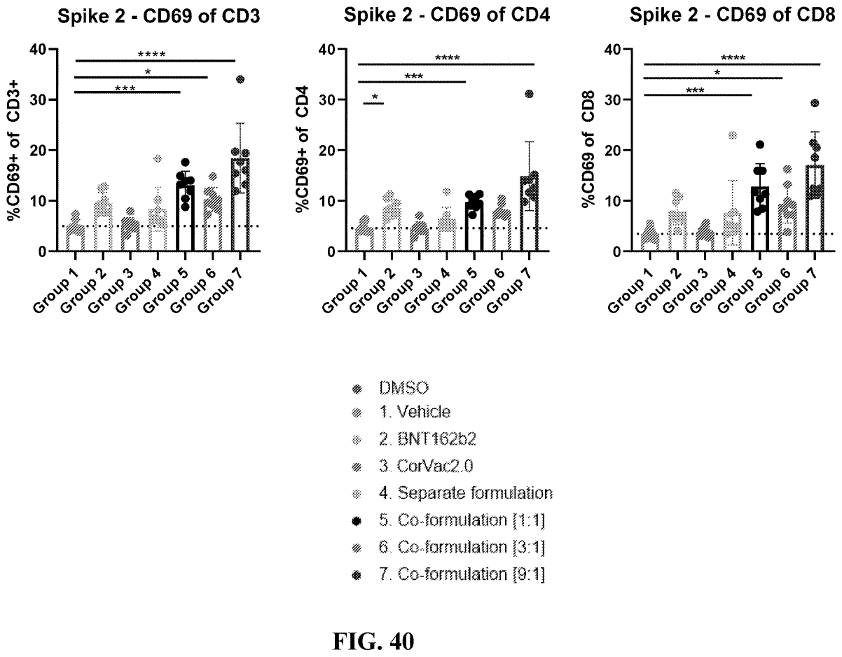

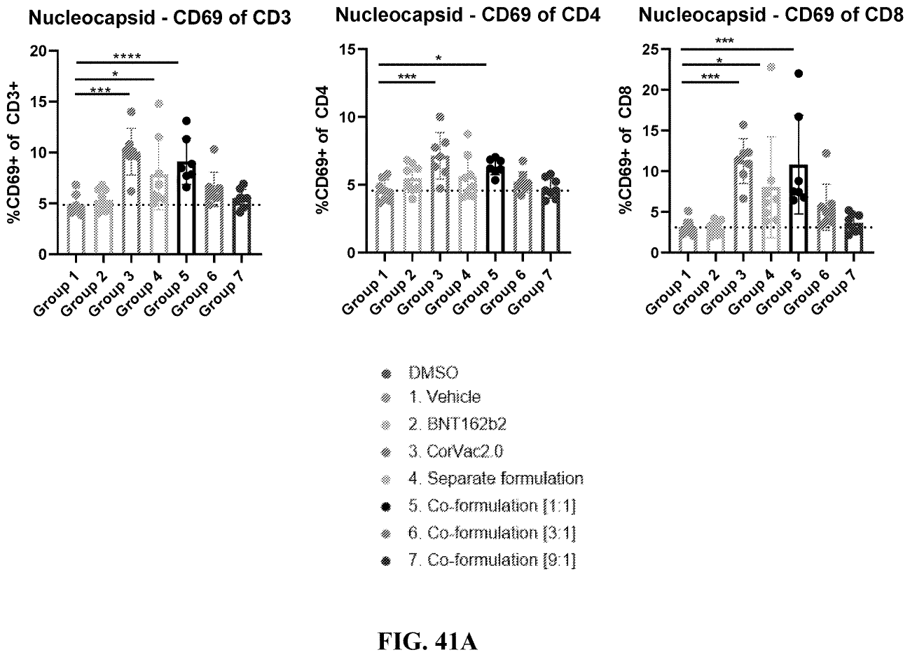

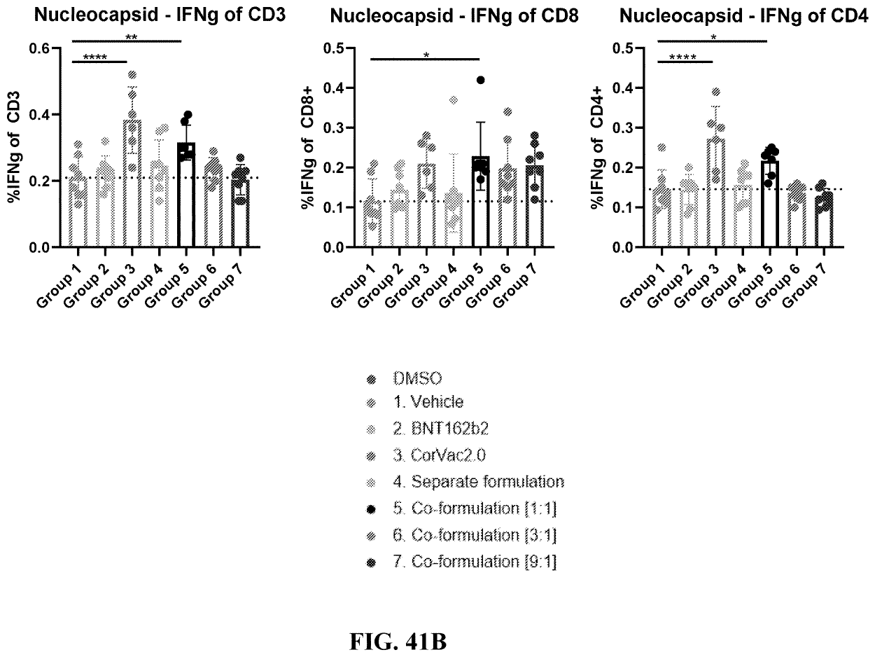

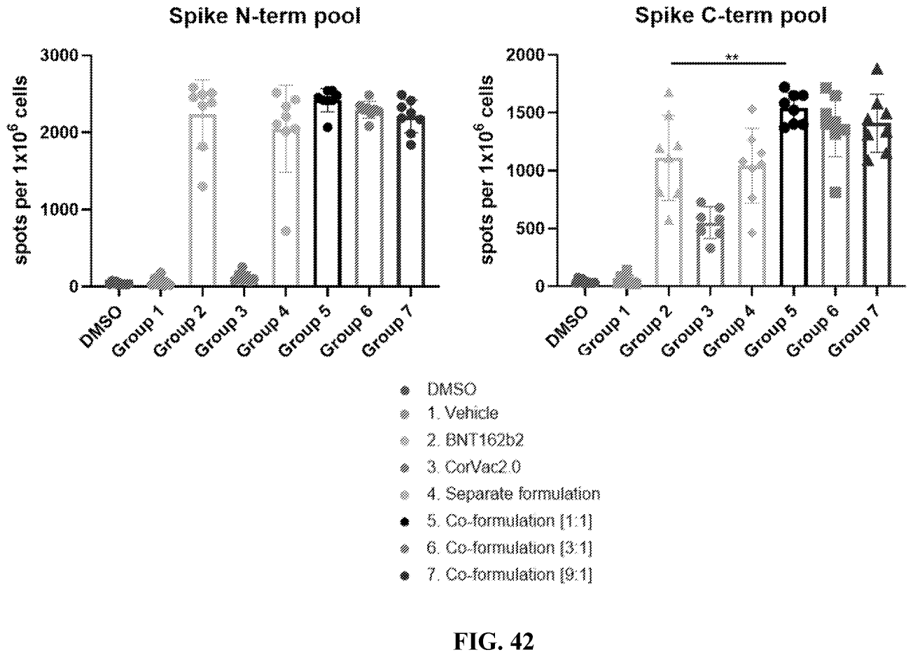

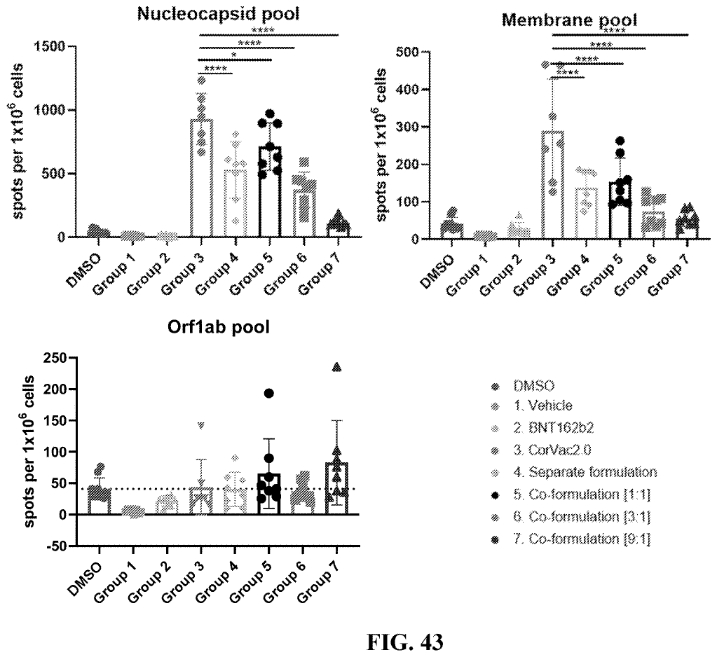

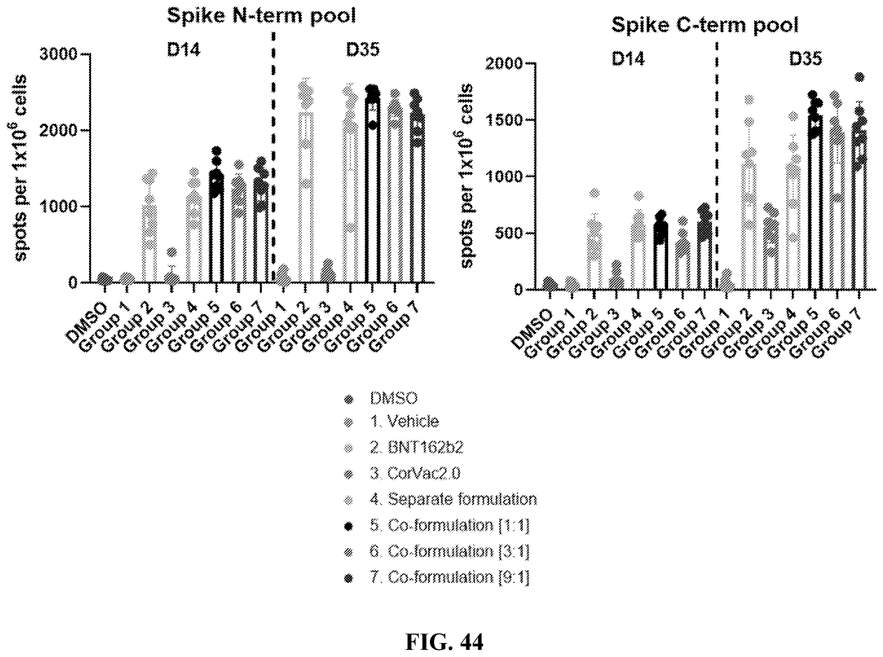

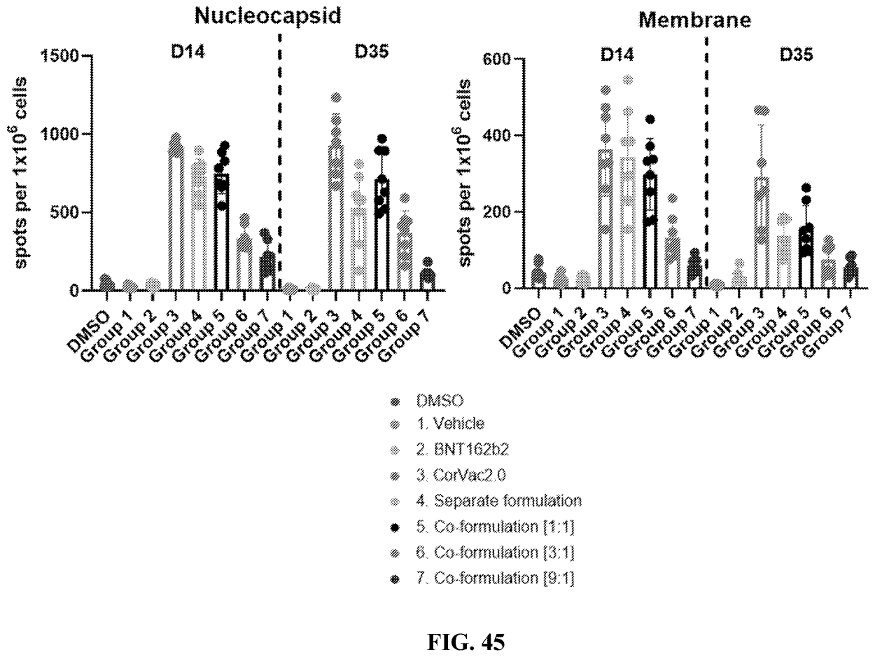

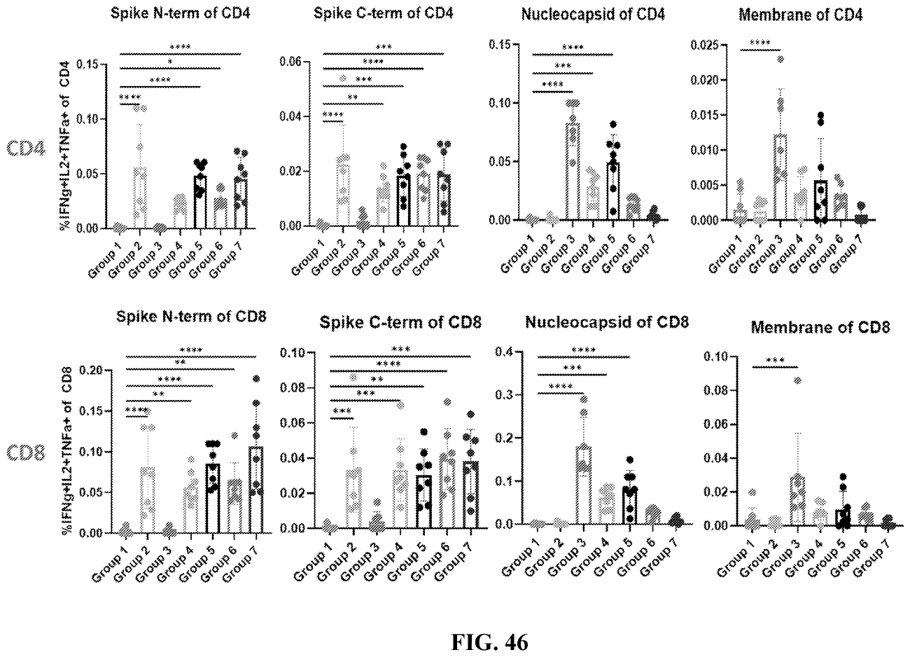

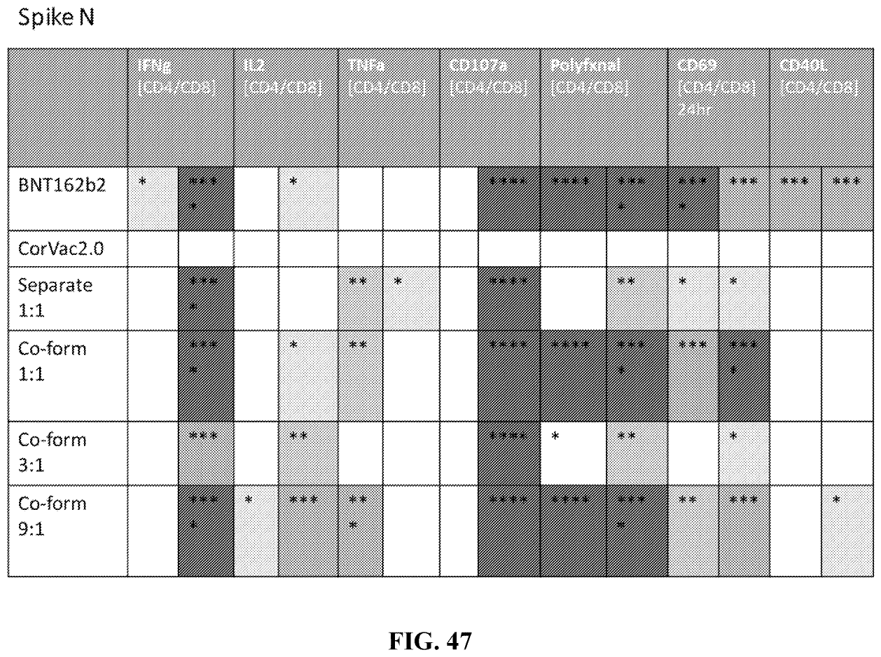

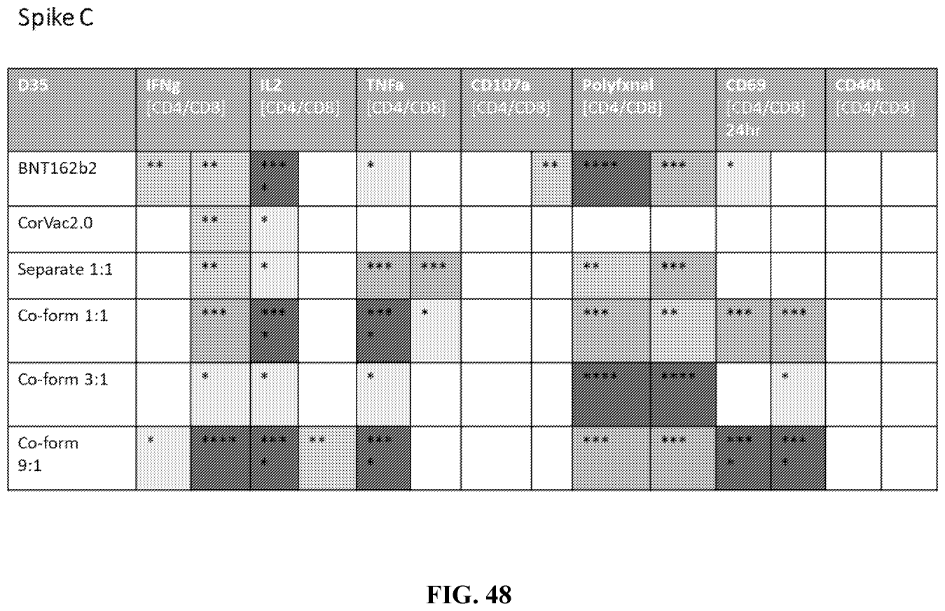

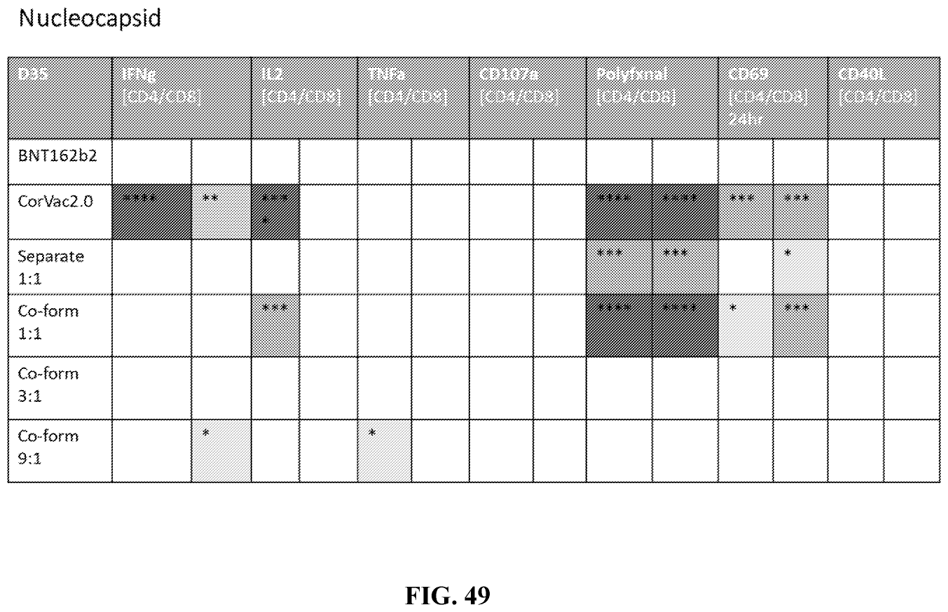

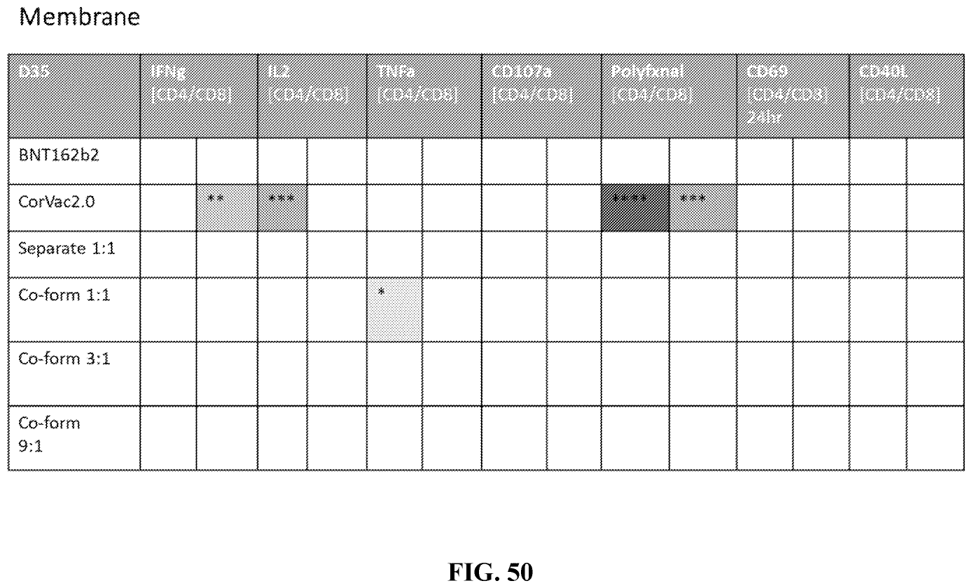

It was found that the B cell response was primarily driven by BNT162b2 directed against the Spike (S1) epitopes (FIG. 34A, FIG. 34B). No serum antibody response was evident against nucleocapsid (NP) in CorVac 2.0 treated mice. However, as demonstrated herein, inclusion of CorVac 2.0 in a co-formulation setting does not impact Spike-specific Ab development (FIG. 35). Additionally, neutralizing antibody titers were not negatively impacted by CorVac 2.0 (FIG. 36), indicating that CorVac 2.0 can be administered to a population that has received BNT162b2 (or other antigen-stimulating vaccine) and/or has been exposed to and/or infected by SARS-CoV-2. In fact, B cell responses are heavily driven by BNT162b2, but addition of CorVac 2.0 [1:1] co-formulation slightly enhances responses (FIG. 37A and FIG. 37B). Ag-specific Spike T-cell responses were not inhibited by addition of CorVac 2.0 (FIG. 42). Nucleocapsid and membrane specific CorVac 2.0 T cell responses were maximal in mice treated with CorVac 2.0 alone (FIG. 43, Group 3). Ag-specific Spike T cell responses for all groups are shown in FIG. 44. Nucleocapsid and membrane specific CorVac 2.0 T cell responses for all groups are shown in FIG. 45. The studies confirmed that both BNT162b2 and CorVac 2.0 lead to polyfunctional responses in CD4 and CD8 T cells, as exemplified by number of CD4 T cells and CD8 T cell isolated from lymph node analysis considering, for example, interferon release, IL-2 release and expression of activation markers (FIG. 46, FIG. 47, FIG. 48, FIG. 49 and FIG. 50).

### Example 19. Immunogenicity Studies of CorVac 2.0 C-7 String with BNT162b2 in HLA-A2 Transgenic Mice

In another study design as exemplified in FIG. 11B, different vaccine strategies are compared in HLA-A2 transgenic mice to test the impact of CorVac 2.0 on anti-Spike antibodies induced by BNT162b2, test CorVac 2.0 T cell responses after prime and boost (2 shots) (e.g., for ORF1ab), compare combinations of BNT162b2 and CorVa c2.0 either through separate formulation (different LNPs) or co-formulation (same LNP), test varying ratios of BNT162b2: CorVac 2.0, keeping BNT162b2 dosage fixed to not reduce Ab titers, and to test if separate formulation is better when given in the same site (leg) or different sites (legs). As exemplified in FIG. 11B, the different vaccine strategies in HLA-A2 transgenic mice for this study include: (a) the spike mRNA vaccine alone, (b) the string vaccine alone at a first dosage (1 microgram), (c) the string vaccine alone at a second dosage (0.3 micrograms), (d) a first formulation of the spike mRNA vaccine and a separate second formulation of the string vaccine in a first ratio (e.g., 3:1) administered on the same day, (e) a first formulation of the spike mRNA vaccine and a separate second formulation of the string vaccine in a second ratio (e.g., 1:1) administered on the same day, (f) a co-formulation of the spike vaccine and the string vaccine in a first ratio (e.g., 3:1); and (g) a co-formulation of the spike vaccine and the string vaccine in a second ratio (e.g., 1:1). In this study, about half of the mice of each set are sacrificed on day 14, and the rest on day 35. Samples are collected at day 0, 14, 21 and 35 after the first injection. ELISPOT is performed, cytokine and chemokine generation is analyzed, and T cell subsets are tested for antiviral cytokine responses, surface marker expression, and expression of lineage markers is analyzed by flow cytometry. Antigen specific responsiveness of T cells is also measured in vitro. Tissue samples are also tested for presence of spike antibody, T cell responses and T cell subsets in spleen, draining lymph nodes and blood.

### Example 20. Immunogenicity Studies of CorVac 2.0 C-7 String with BNT162b2 in Transgenic Mice Expressing Human ACE2

In another study design as exemplified in FIG. 11C, different vaccine strategies are compared in transgenic mice expressing human ACE2 (human version of the receptor SARS-CoV-2 uses to infect cells) to examine CorVac 2.0 as a booster after BNT162b2, test the effects of how a third shot of BNT162b2, CorVac 2.0 or the combination of both (e.g., as a co-formulation, separate formulations, and different BNT162b2: CorVac 2.0 ratios) in mice that have already received BNT162b2 prime and boost, and test immune responses using repeated injections of combined BNT162b2+CorVac 2.0 (e.g., may simulate giving the combination to previously unvaccinated individuals). As exemplified in FIG. 11C, the different vaccine strategies in HLA-A2 transgenic mice for this study include: (a) 2 shots of the spike mRNA vaccine alone (b) 3 shots of the spike mRNA vaccine alone, (c) 2 shots of the spike mRNA vaccine alone followed by 1 shot of a co-formulation of the spike vaccine and the string vaccine in a first ratio (e.g., 3:1) (d) 2 shots of the spike mRNA vaccine alone followed by 1 shot of the string vaccine alone, (e) 2 shots of the spike mRNA vaccine alone followed by a first formulation of the spike mRNA vaccine and a separate second formulation of the string vaccine in a first ratio (e.g., 3:1) administered on the same day, (f) 1 shot of the spike mRNA vaccine alone followed by administration of a first formulation of the spike mRNA vaccine and a separate second formulation of the string vaccine in a first ratio (e.g., 3:1) on the same day followed by administration of a first formulation of the spike mRNA vaccine and a separate second formulation of the string vaccine in a first ratio (e.g., 3:1) on the same day, (g) administration of a first formulation of the spike mRNA vaccine and a separate second formulation of the string vaccine in a first ratio (e.g., 3:1) followed by administration of a first formulation of the spike mRNA vaccine and a separate second formulation of the string vaccine in a first ratio (e.g., 3:1) on the same day followed by administration of a first formulation of the spike mRNA vaccine and a separate second formulation of the string vaccine in a first ratio (e.g., 3:1) on the same day; and (g) 2 shots of the spike mRNA vaccine alone followed by a first formulation of the spike mRNA vaccine and a separate second formulation of the string vaccine in a second ratio (e.g., 9:1) administered on the same day. In this study, mice are sacrificed on day 56. Samples are collected at day 0, 14, 21, 35, 42 and 56 after the first injection. ELISPOT is performed, cytokine and chemokine generation is analyzed, and T cell subsets are tested for antiviral cytokine responses, surface marker expression, and expression of lineage markers is analyzed by flow cytometry. Antigen specific responsiveness of T cells is also measured in vitro. Tissue samples are also tested for presence of spike antibody, T cell responses and T cell subsets in spleen, draining lymph nodes and blood.

### Example 21. Effect of CorVac 2.0 on Spike Antibody and Anti-Spike T Cell Response

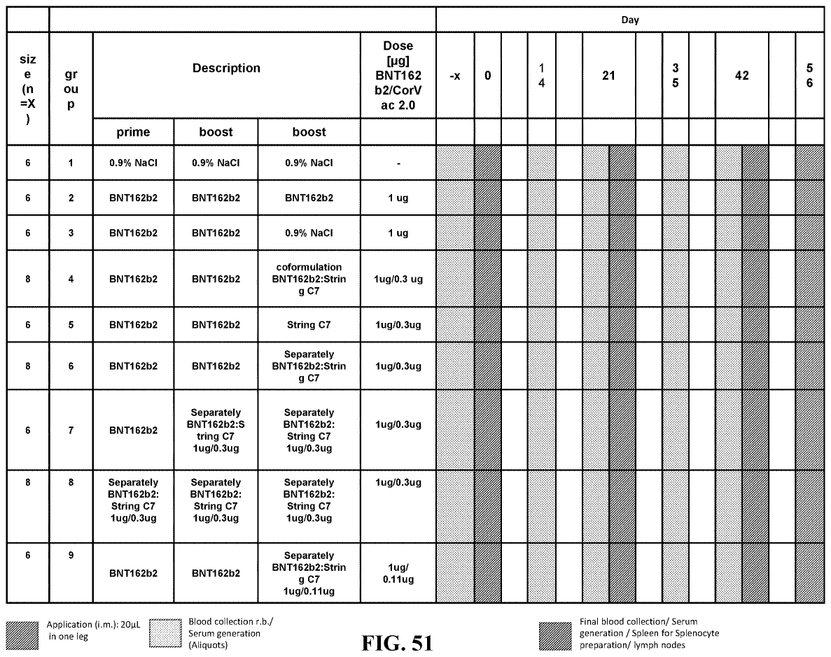

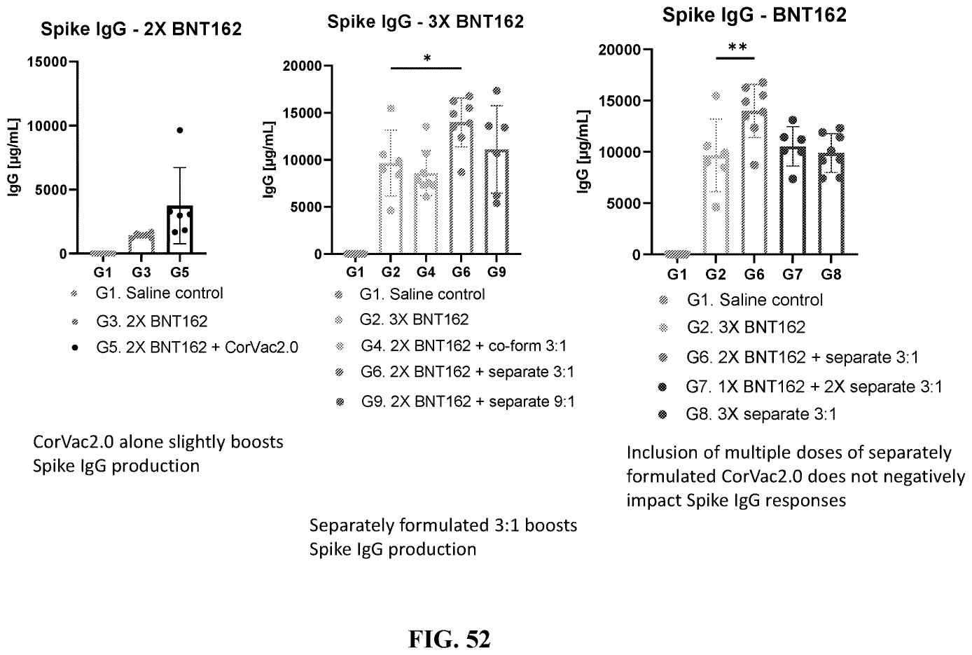

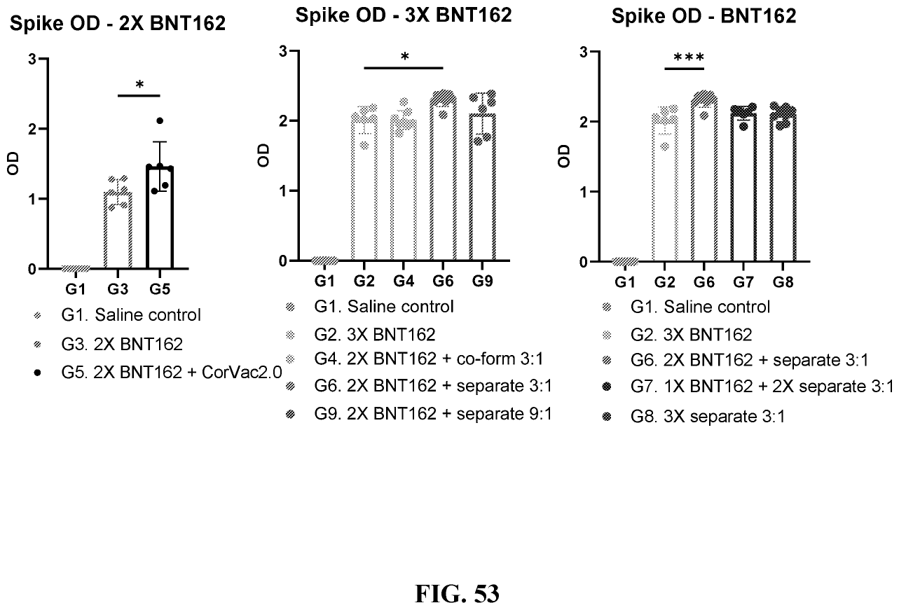

This study was designed to evaluate the effects of CorVac 2.0 strings on antibody levels of spike protein from prior administered BNT162b2 vaccine. Nine groups of mice with 6 mice each were administered a prime-boost-boost regimen as shown in FIG. 51. C7 string was used as exemplary of the CorVac 2.0 strings. Vaccines were administered intramuscularly in a 20 microliter solution in one leg. Retroorbital bleeding was performed to collect blood on a day prior to the first vaccine application (prime), and on days 14, 21, 35, 42 and 56 from prime. On day 56 mice were sacrificed, organs harvested, splenocytes and lymph node preparations were made. Data shown in FIG. 52 indicates that separately formulated CorVac2.0 at 0.3 ug dose enhances spike IgG. CorVac2.0 (e.g., C7 string) alone slightly boosts Spike IgG production (FIG. 52 left graph). Separately formulated (Group 6, G6) 3:1 boosts Spike IgG production (FIG. 52 middle graph). Inclusion of multiple doses of separately formulated CorVac2.0 does not negatively impact Spike IgG responses (FIG. 52, right graph). Similar results were observed in FIG. 53 with optical density measurement of Spike antigen specific antibody levels.

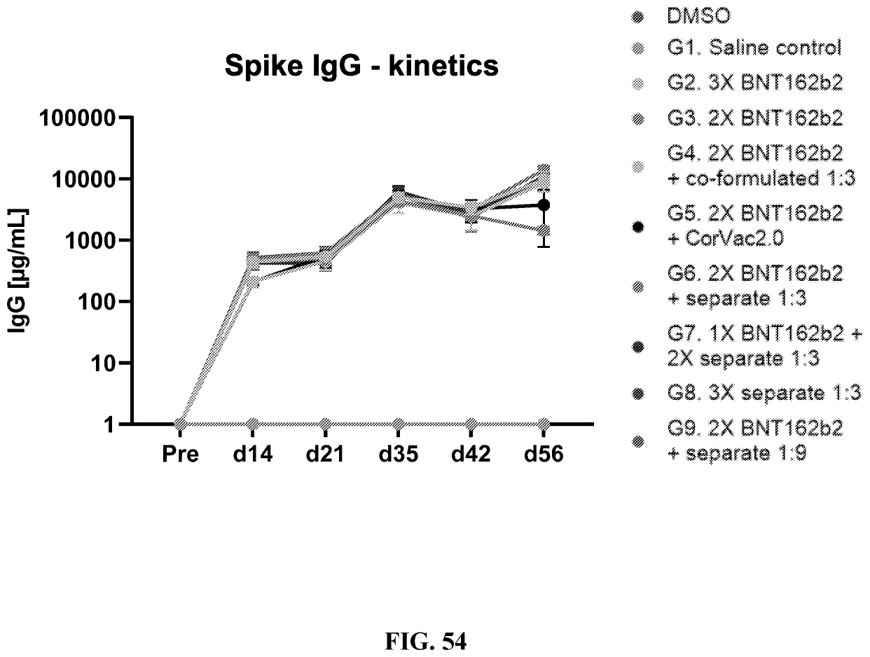

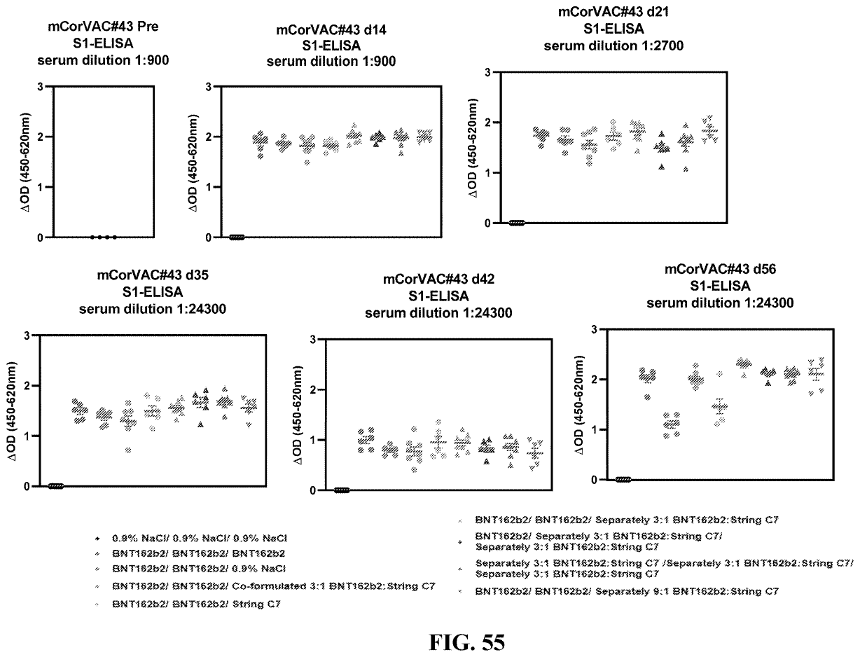

Spike antibody titer increases with time after administration of BNT162b2 alone (e.g., in Groups 2 and 3; FIG. 54 G2, G3) over 42 days post vaccination, using the regime shown in FIG. 51. With the various separately formulated doses of Corvac 2.0 boosts as shown in the regime of FIG. 51, spike antibody titer continues to remain high, and shows higher titers at day 56, compared to a slight decrease in the same for BNT162b2 treatment alone (FIG. 54). Particularly, third boost increases spike IgG with all Corvac 2.0 variations, as demonstrated in FIG. 54. FIG. 55 shows spike (S1) ELISA results at the indicated dilutions.

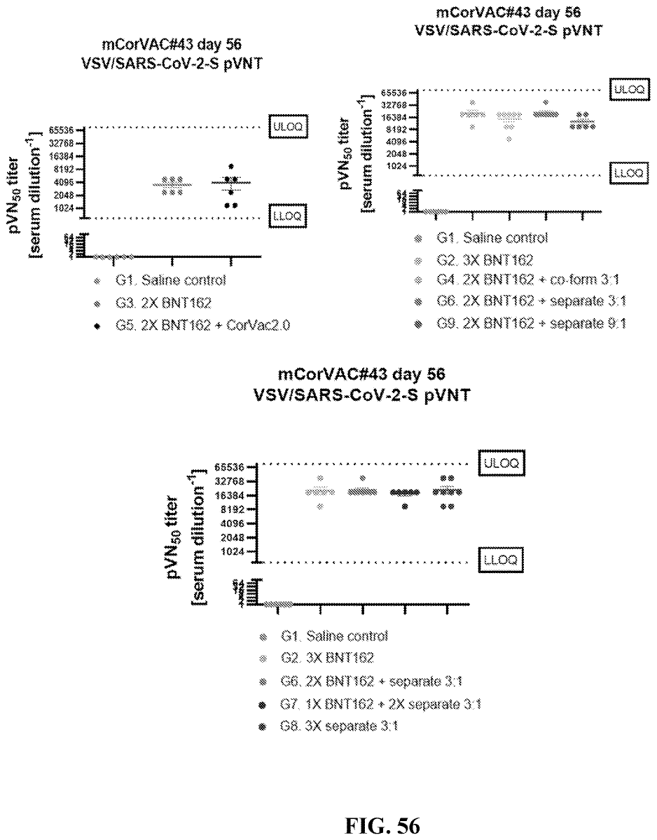

Additionally, data shown in FIG. 56 shows that inclusion of CorVac 2.0 does not impact neutralizing antiviral antibody titers negatively. Pseudovirus neutralizing titers against VSV/SARS-CoV-2S are not reduced.

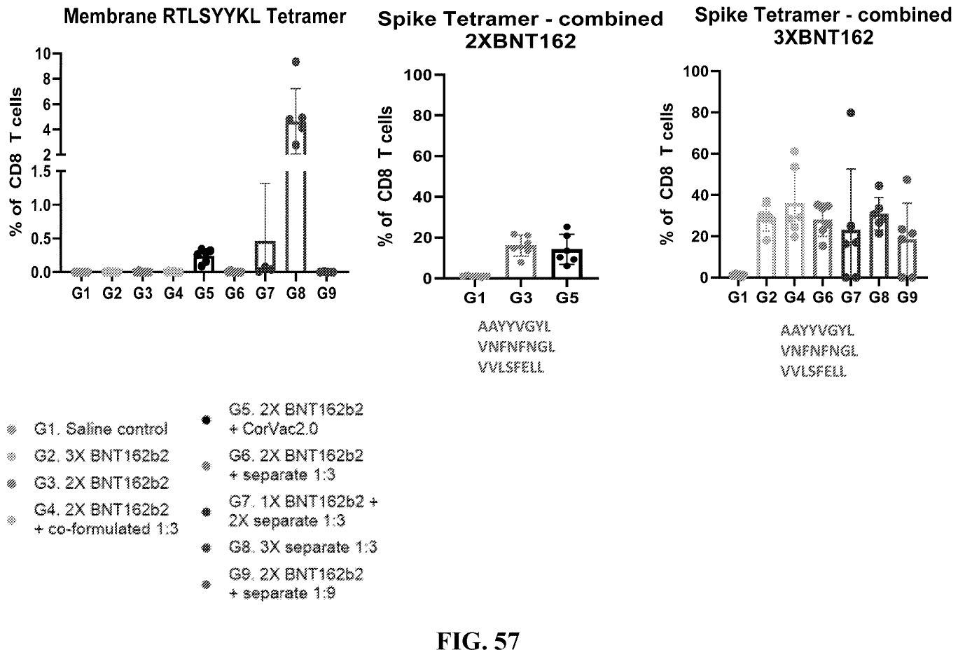

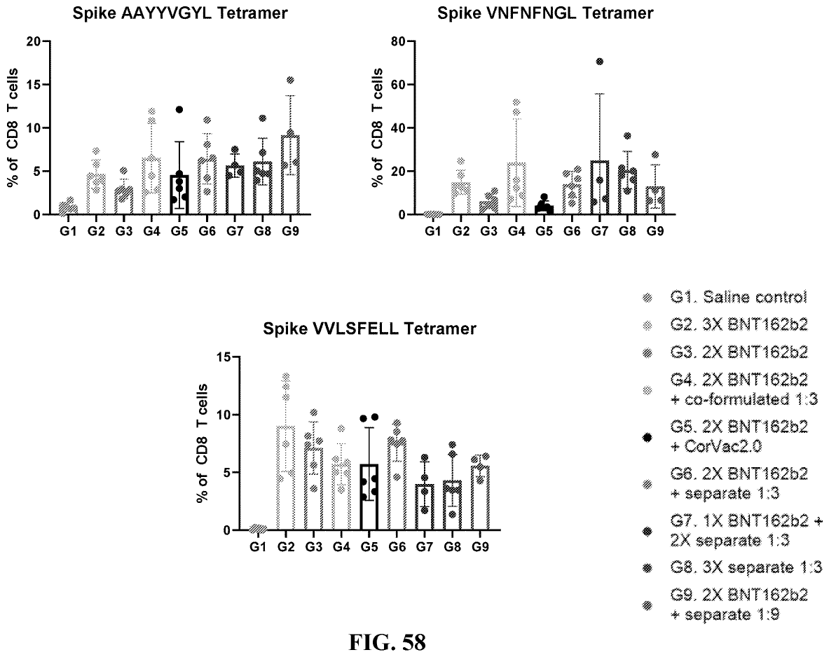

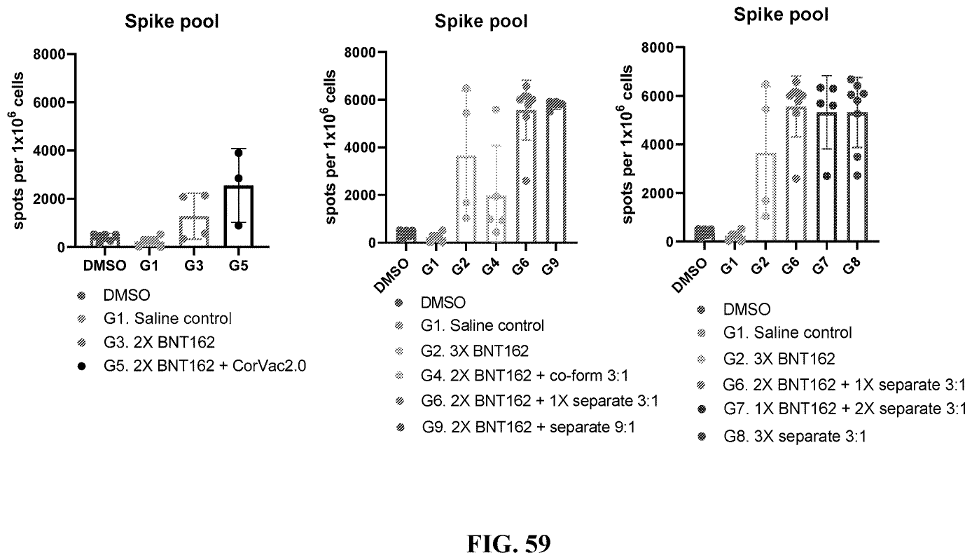

T cell responses were evaluated by tetramer staining. Tetramer staining of peripheral blood leukocytes show that Spike responses are not impacted by addition of CorVac 2.0 (FIG. 57). Likewise, FIG. 58 shows T cell responses against three different Spike epitopes AAYYVGYL, VNFNENGL and VVLSFELL. CorVac2.0 alone slightly boosts Spike T cell responses (FIG. 59, left graph); while separately formulated 3:1 and 9:1 slightly boost Spike T cells (FIG. 59, middle graph). Multiple doses of separately formulated CorVac 2.0 does not negatively impact Spike T cell responses (FIG. 59, right graph). In fact, addition of CorVac 2.0 in the 3^{rd} booster shot as a separate formylation slightly improves T cell responses to Spike.

These results demonstrate that boosting with CorVac2.0 only, or with separately formulated CorVac2.0+BNT162b2 leads to increased Spike IgG titers, but does not affect neutralizing titers. Of the three epitopes tested, combination with CorVac2.0 does not impact tetramer-specific PBLs.

### Example 22. T Cell Responses to Other SARS-CoV-2 Proteins with BNT162b2+CorVac 2.0 Vaccination

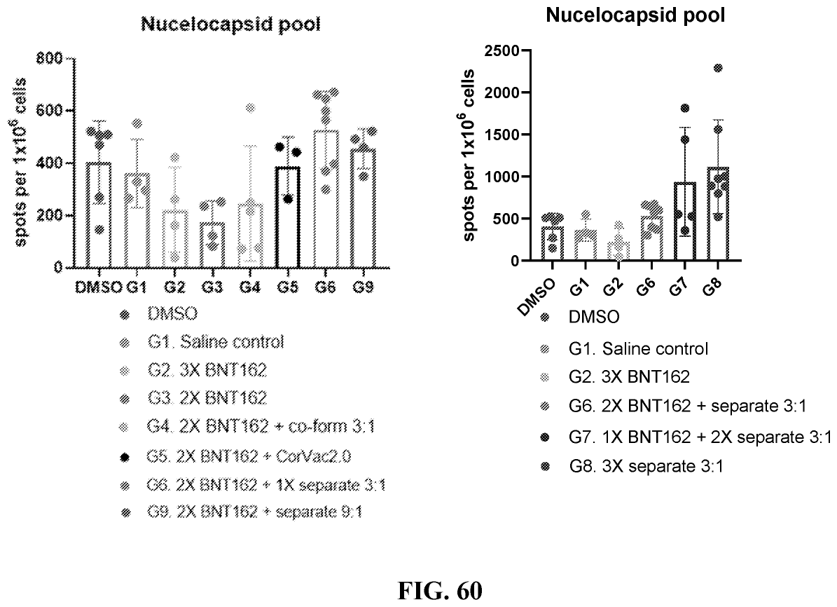

From the study outlined in FIG. 51, T cell responses to Spike and CorVac 2.0 associated proteins (e.g. Membrane protein) were analyzed when BNT162b2 and CorVac 2.0 were co-administered as a third booster. Data presented in FIG. 60 indicate that when used in combination with BNT162b2, antibody and T cell responses to spike are maintained as well as additional T cells specific for additional SARS-CoV2 protein associated epitopes are evidenced.

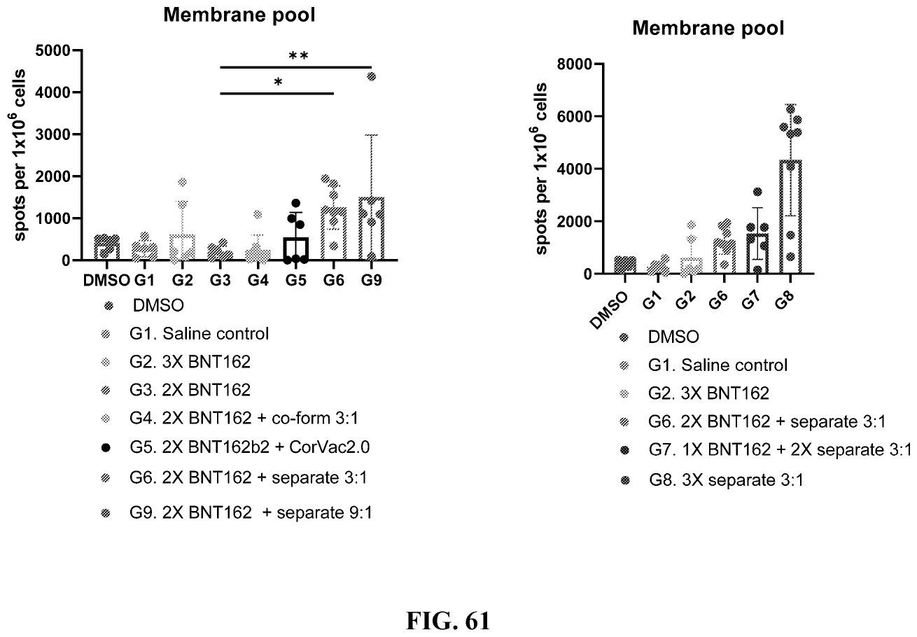

T cell response to viral nucleocapsid is a weak response observed when tested in hACE2 mice after one boost dose with CorVac 2.0 (FIG. 60, left graph, G4, G5, G6, G9). However, multiple doses of separately formulated BNT 162b2+CorVac 2.0 leads to increases in the response (FIG. 60, right graph, G7, G8). Response to the viral membrane (M) protein was observed to be the strongest when seen in separately formulated groups as booster (FIG. 61, left). Also, multiple doses of separately formulated BNT162b2+CorVac 2.0 leads to increase in the responses (FIG. 61, right).

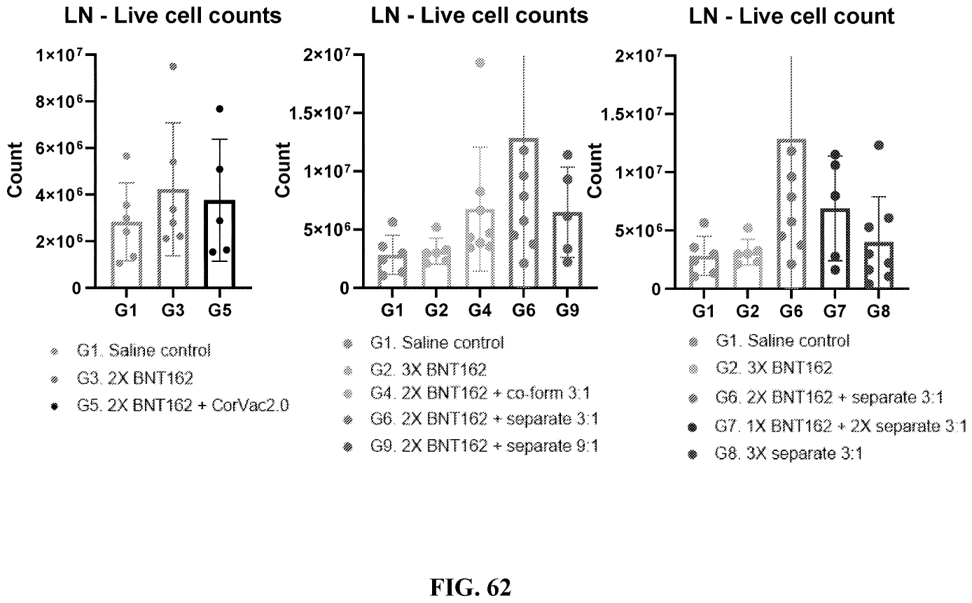

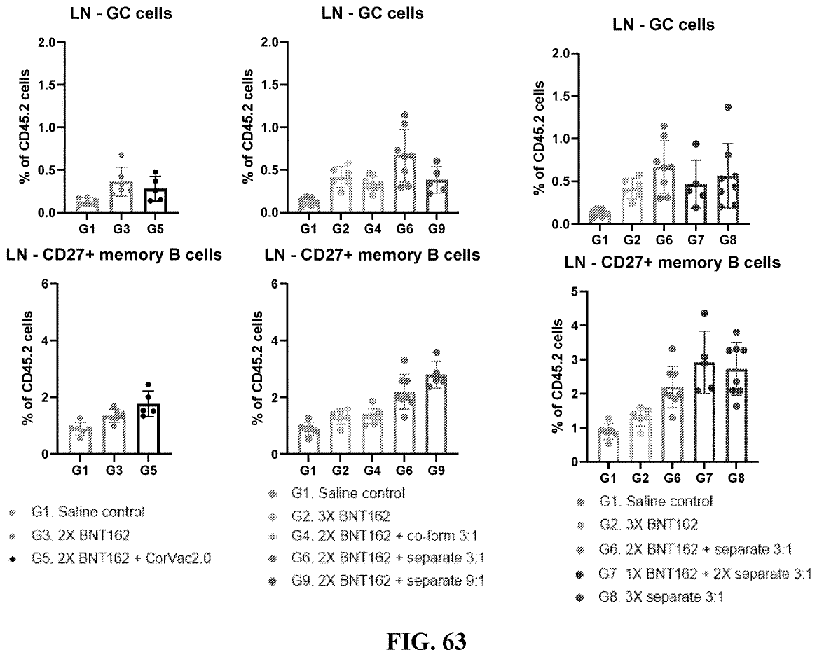

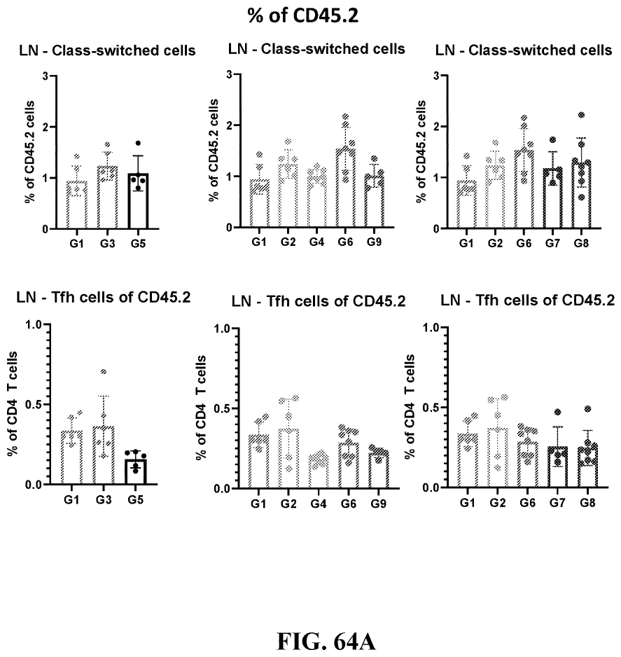

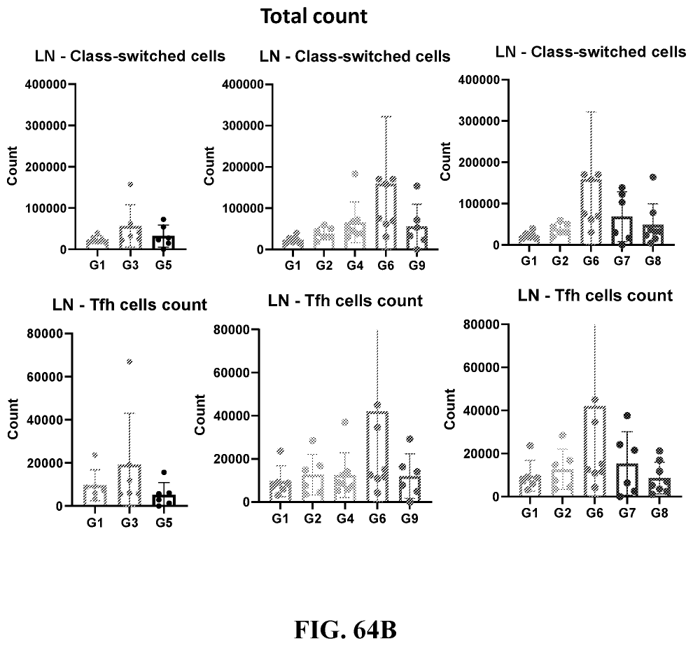

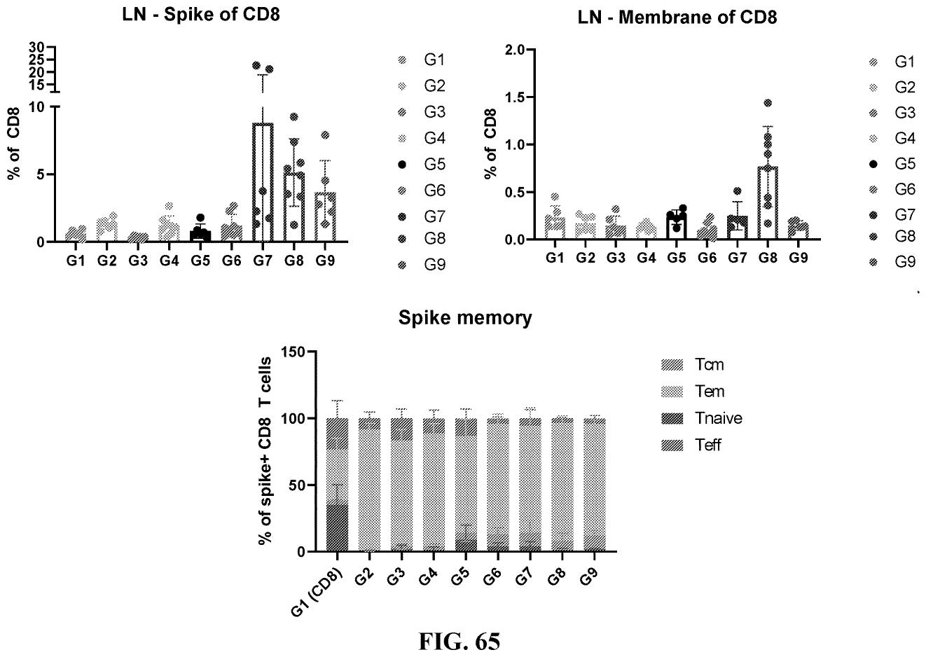

Lymph nodes were analyzed for T cell responses to the various treatments outlined in FIG. 51. Cell counts increased in the lymph nodes (LN), indicative of cellular proliferation (FIG. 62). The LN cells were then phenotyped. Increased GCs and CD27+ memory B cells were observed in the groups that was administered separately formulated CorVac 2.0+BNT162b2 (G4-G9) (FIG. 63). A slightly higher B cell class switching was observed in the groups receiving the seperately formulated BNT162b2+CorVac 2.0 (FIGS. 64A and 64B). The cell types found in the LN are further analyzed as demonstrated in FIG. 65, showing spike-specific CD8 memory cells as % of CD8+ T cells (top left), membrane-specific CD8 memory cells as % of CD8+ T cells (top right), and proportions of T cells that are effector, memory or naïve as % of spike-specific CD8+ T cells (bottom).

These results indicate that separately formulated CorVac2.0+BNT162 as a third booster leads to high Spike and Membrane T cell responses. Nucleocapsid responses did not appear as high in the K18-hACE2 model. Separately formulated CorVac2.0+BNT162 as a third booster leads to increased GC B cells and memory B cells in the dLNs. Spike-specific T cells in the dLN appear less differentiated in groups including CorVac2.0

### Example 23. Separately Formulated BNT162b2 and Cor Vac 2.0 Study in HLA.A2 Tg Mice

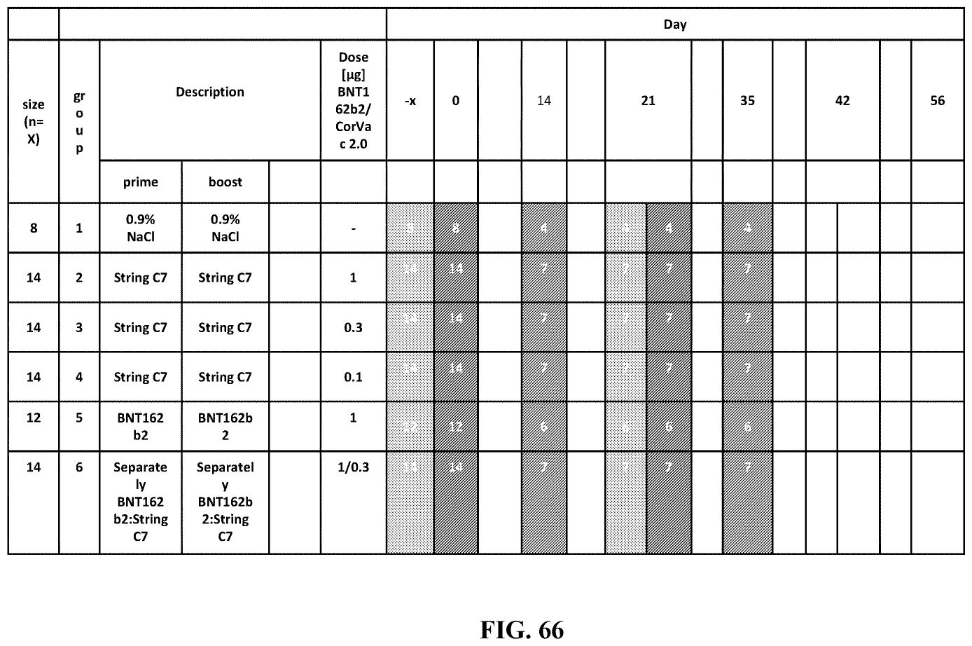

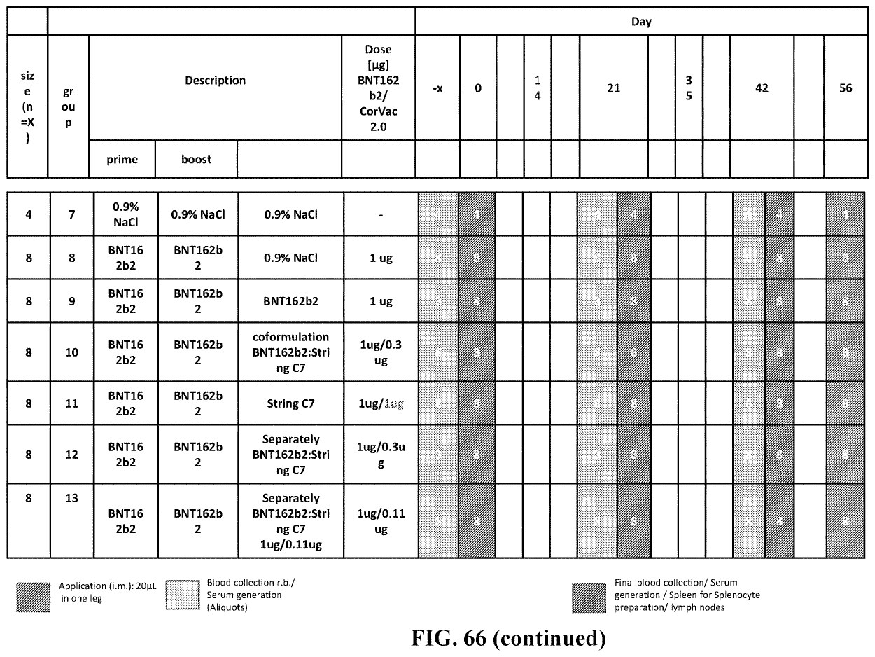

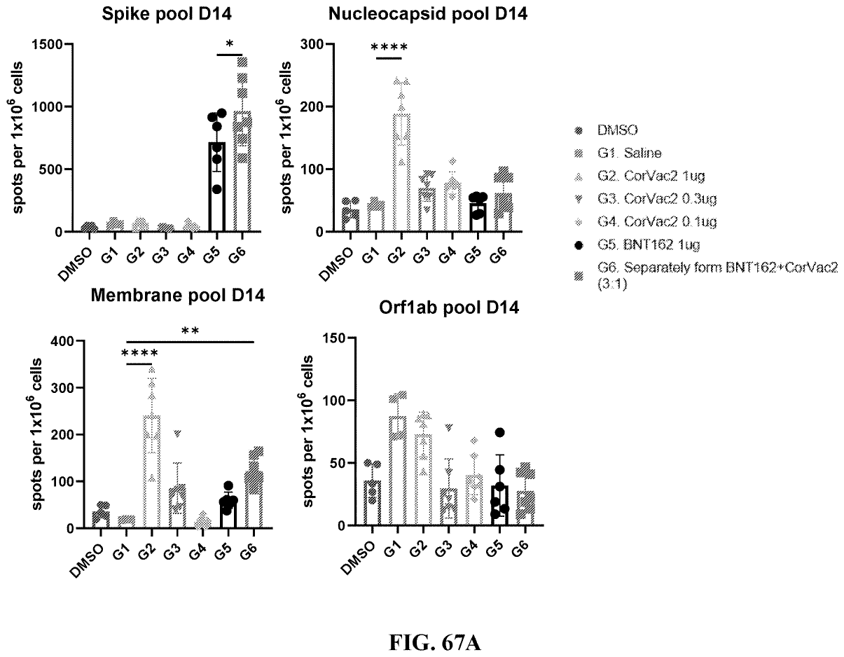

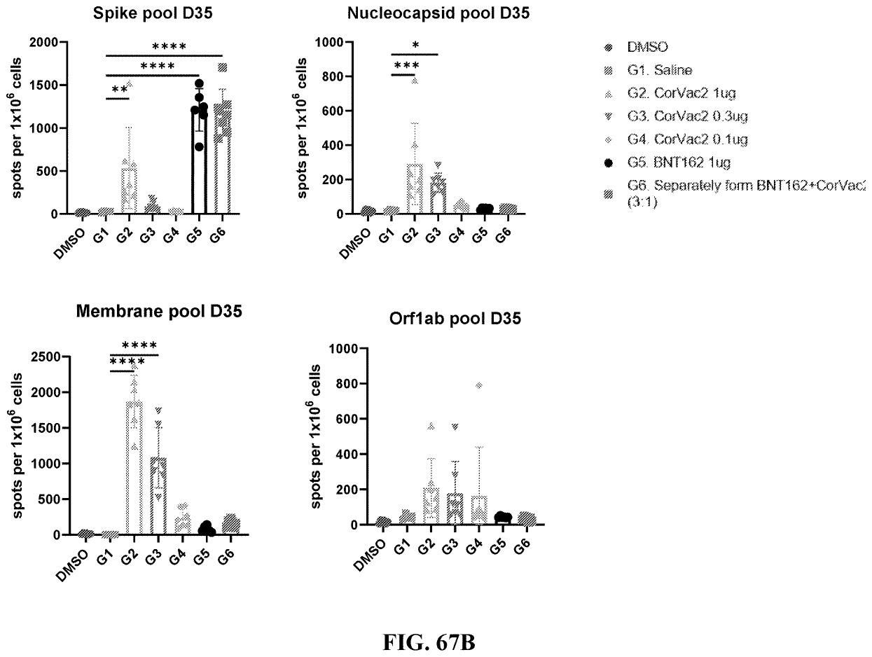

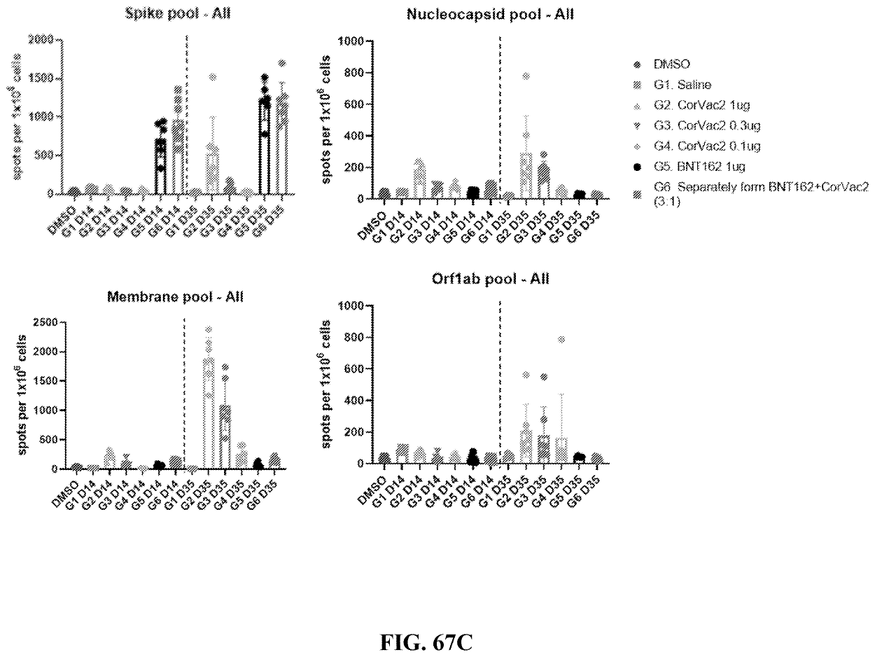

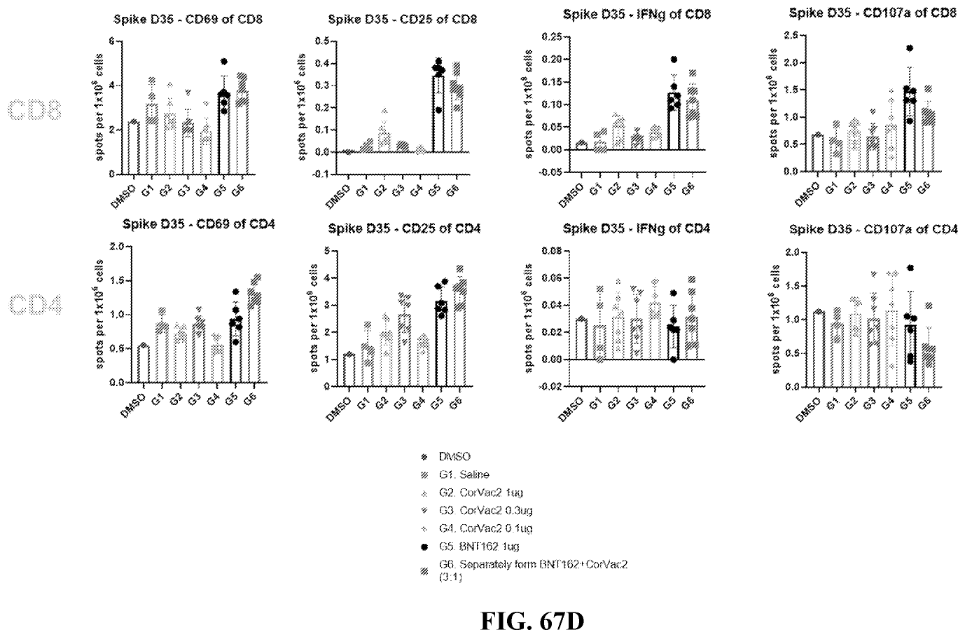

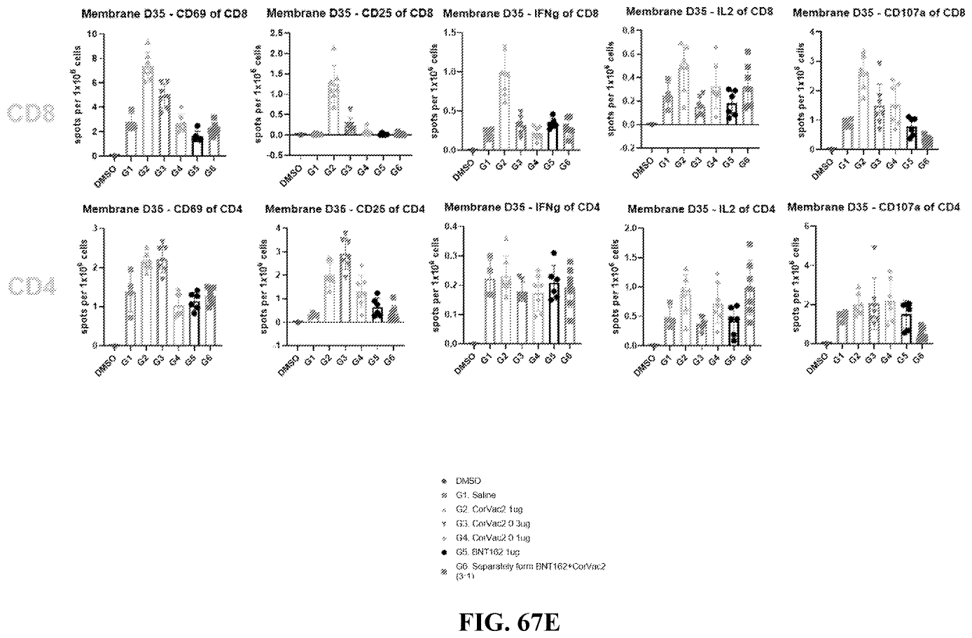

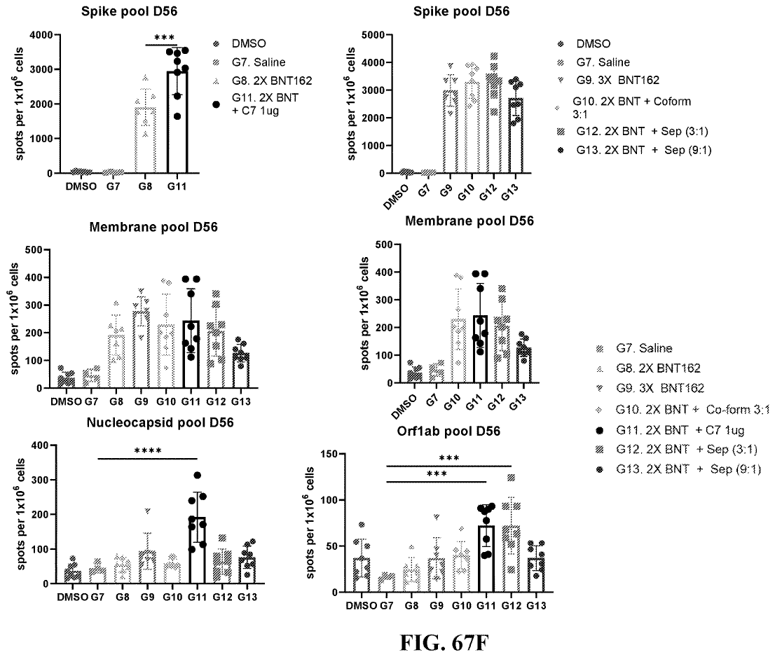

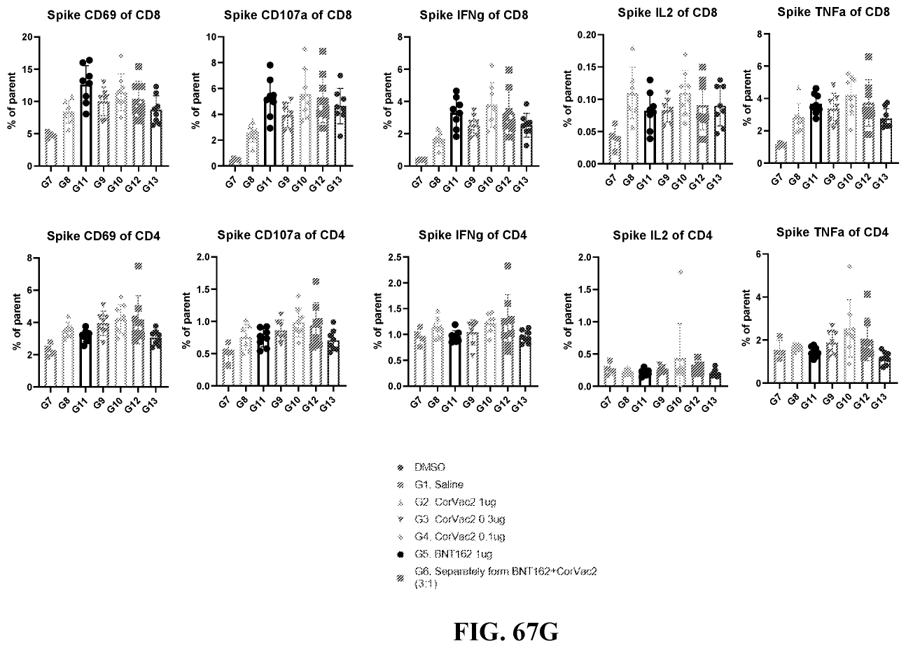

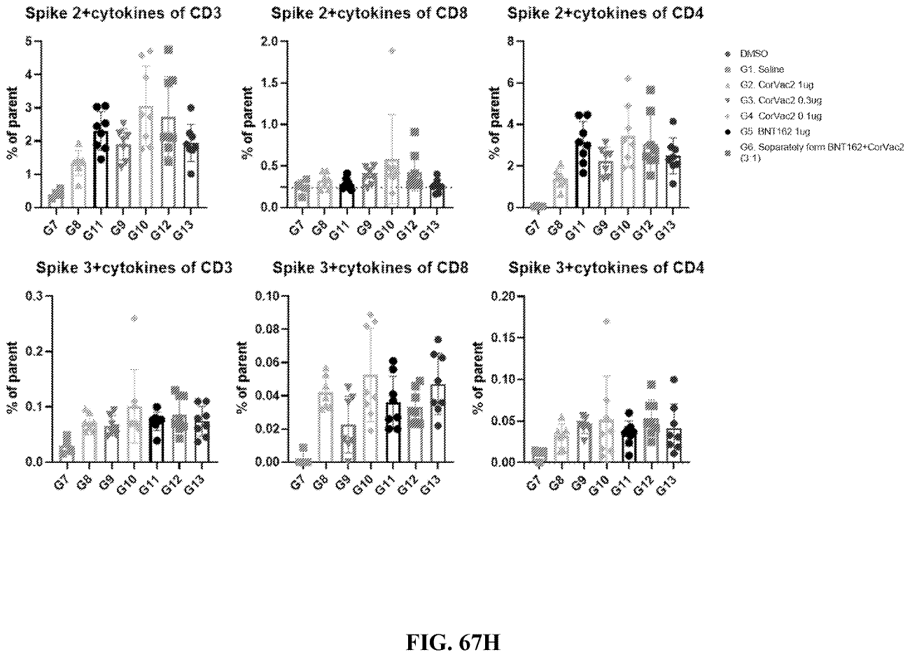

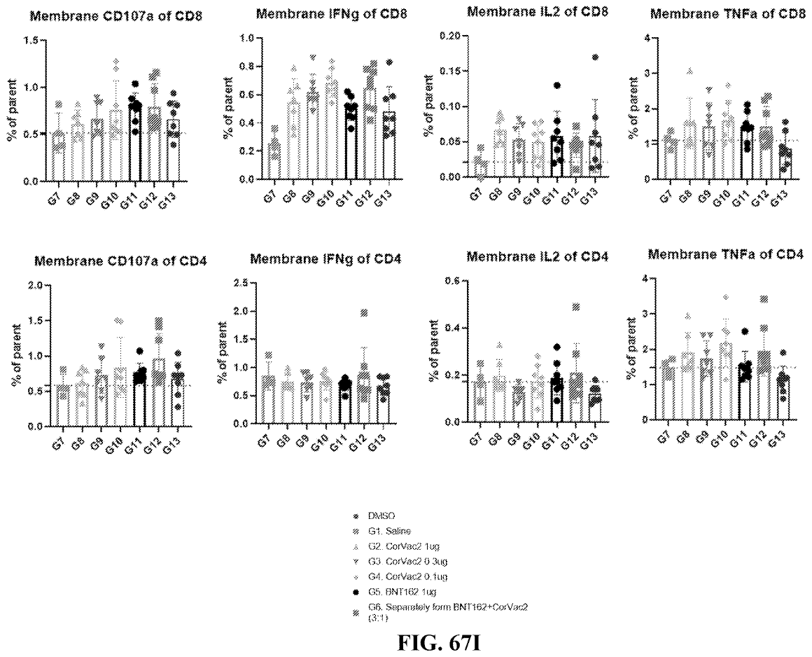

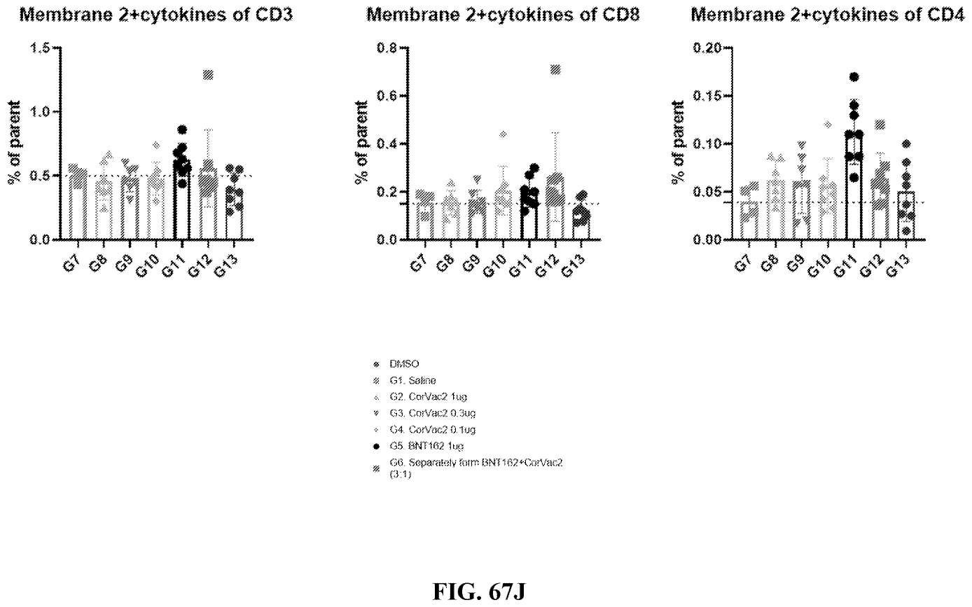

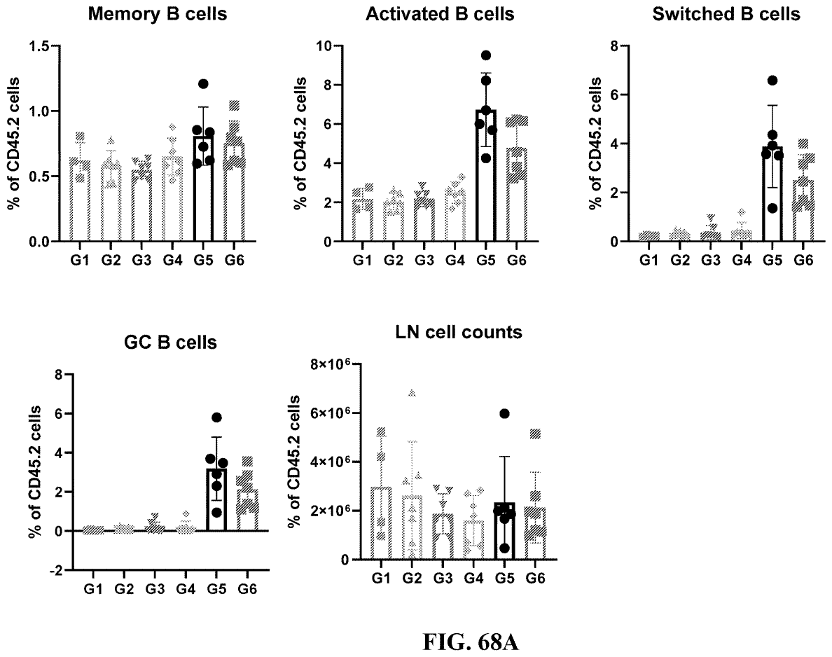

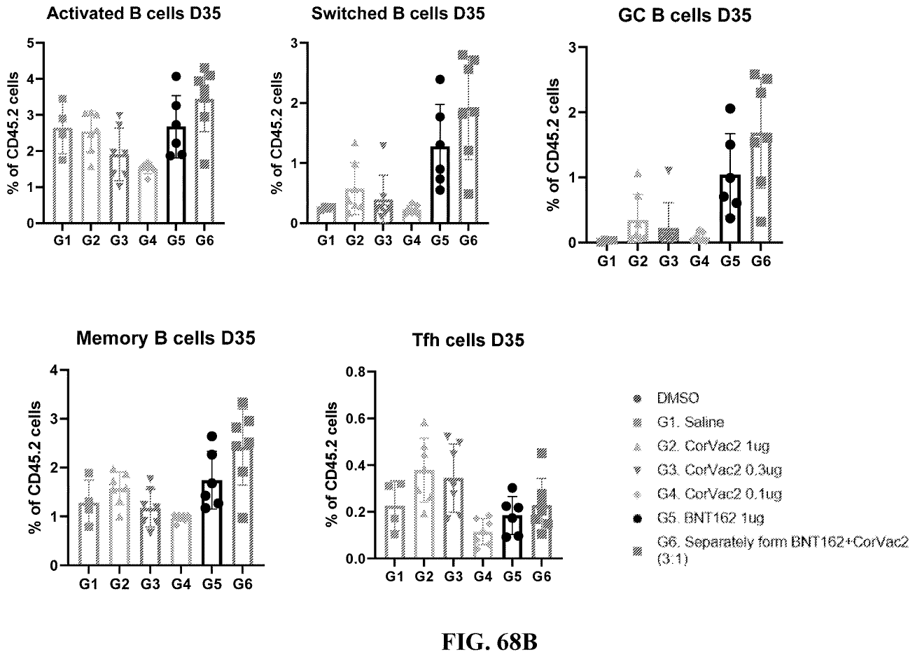

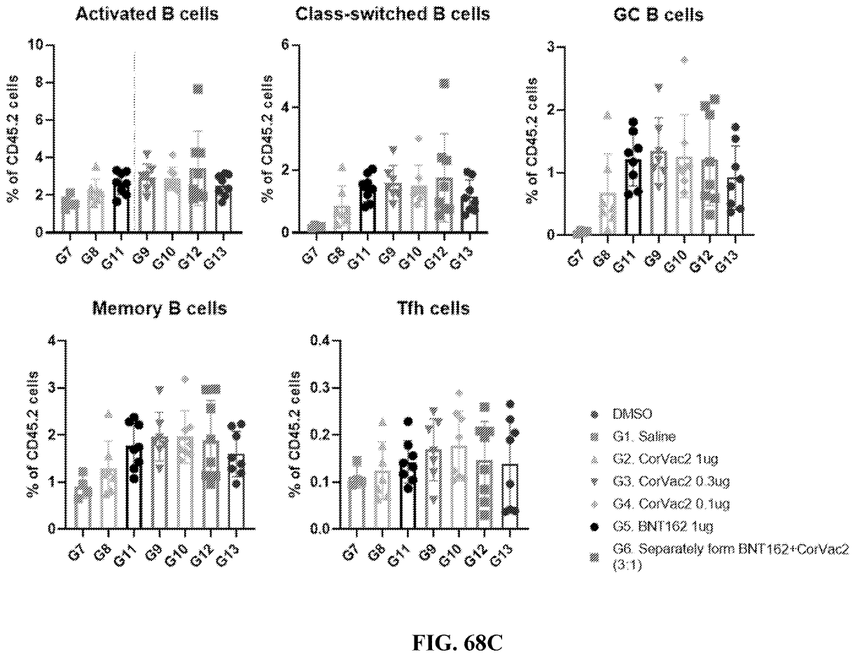

In this example, HLA.A2 transgenic (HLA.A2Tg) mice were vaccinated with the combination of BNT162b2 and CorVac 2.0 in a study design regime shown in FIG. 66 The design consisted of 13 groups of 8-14 mice per group as shown, that were primed with CorVac 2.0 C7 string as exemplary of CorVac 2.0 vaccines, boosted with C7 (groups 2-4; G2-G4); primed with BNT162b2 and String C7 seperately formulated and were administered the same as prime for boost (G6); prime and 2 boosts both with BNT162b2 (G8) or with the 3^{rd} dose or saline (G7), prime and boost with BNT162b2, with a coformulation of BNT162b2 and String C7 for a third dose (G10); and prime and boost with BNT162b2, with a separate formulation of BNT162b2 and String C7 for a third dose (G 12, G13). Data obtained from this study (FIG. 67A-J) showed that CorVac 2.0 induces the stringest immune response at highest dose. Lymph node cells were phenotyped. Data indicate that separately formulated BNT162b2 and String vaccine showed high Spike-specific response in G6 on D14, and Membrane protein specific response was detectable over the threshold by day 14, 35 and 56 (FIG. 67A, B and F), with high memory B cells, presence of activated B cells and class switching (FIG. 68A-C). The data in FIG. 67B demonstrates that Spike T cell responses are enhanced with CorVac2.0 inclusion and the dose of 1 microgram yielded the strongest responses. The data in FIG. 67C demonstrates improved T cell responses to all regions were observed after the boost. The data in FIG. 67D deomnstrates that at day 35, spike responses were seen in CD4 and CD8 T cells and Ssike CD8 T cells show more cytokine secretion and degranulation than CD4 T cells. The data in FIG. 67E deomnstrates that at day 35, membrane responses were seen in CD4 and CD8 T cells and were strongest at the highest dose. IL2 secretion was seen in CD4 and CD8 T cells. The data in FIG. 67F deomnstrates that at day 56, Spike responses were not negatively impacted by CorVac2.0. CorVac2.0 alone boosted spike responses. Boost with CorVac2.0 alone increased spike responses. No statistically significant changes in spike responses were seen with inclusion of CorVac2.0. The strongest membrane responses were observed in the highest dose groups. The strongest N responseswere observed in CorVac2.0 alone at the highest dose. The strongest Orflab responses were observed in CorVac2.0 alone and the separately formulated 3:1 dose. The data in FIG. 67G deomnstrates that at day 56, spike responses were seen in CD4 and CD8 T cells and some were boosted by inclusion of CorVac2.0. The data in FIG. 67H deomnstrates that at day 56, polyfunctional spike CD4 and CD8 T cells were slightly enhanced in higher dose CorVac2.0 conditions. The data in FIG. 67I deomnstrates that at day 56, membrane responses seen more strongly in CD8 T cells. The data in FIG. 67G deomnstrates that at day 56, polyfunctional membrane CD4 and CD8 T cells were observed.

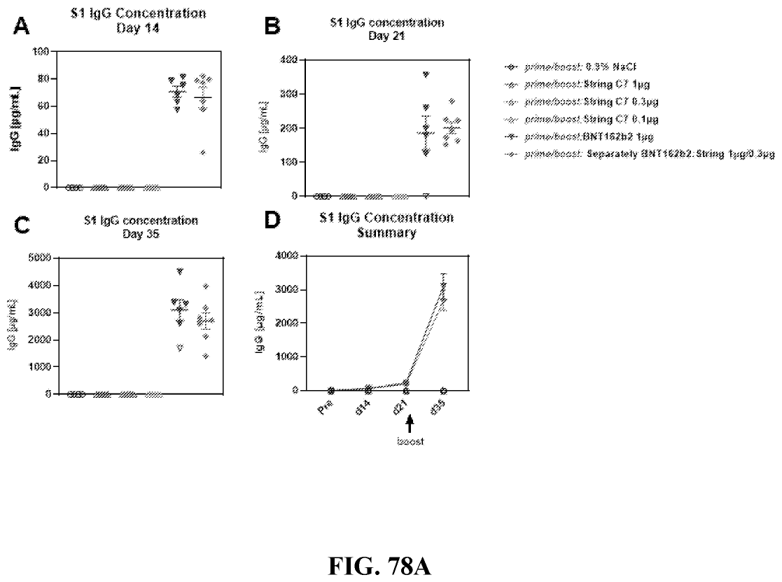

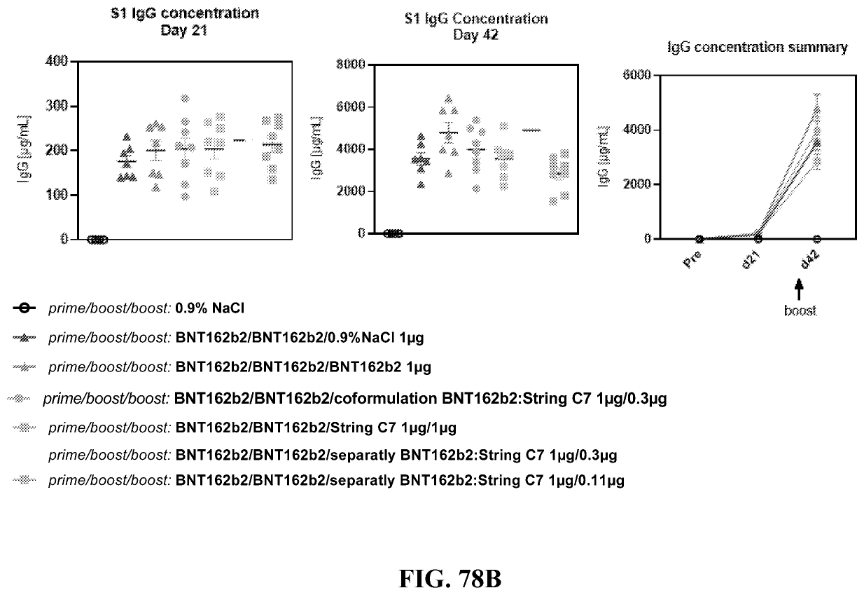

Additionally, as shown in FIG. 78A, serum was taken from mice that were injected with a prime injection at the start of study and boosted on day 21 of the study with various concentrations of RS-C7 (String C7), BNT162b2 or co-administration of BNT162b2 and String C7 at a 3:1 ratio. An ELISA against the S1 portion of the SARS-CoV-2 Spike protein was performed with the serum and the OD was used to determine the S1 specific IgG compared to a standard curve. Values were calculated at day 14 (A), day 21 (B), day 35 (C) and plotted over the length of the study (D). FIG. 78B shows serum was taken from mice that were injected with a prime injection of BNT162b2 at the start of study and boosted with BNT162b2 on day 21 of the study, and subsequently given a third dose of saline (0.9% NaCl), BNT162b2, RS-C7 (string C7), BNT162b2 or co-administration of BNT162b2 and String C7 at a 3:1 ratio and 9:1 ratio, and the RNA from BNT162b2 and RS-C7 co-formulated in the same LNP at a 3:1 ratio. An ELISA against the S1 portion of the SARS-CoV-2 Spike protein was performed with the serum and the OD was used to determine the S1 specific IgG compared to a standard curve. Values were calculated at day 21 (A), day 42 (B) and plotted over the length of the study (C).

### Example 24. Clinical Trial Scheme with Cor Vac 2.0 Vaccine

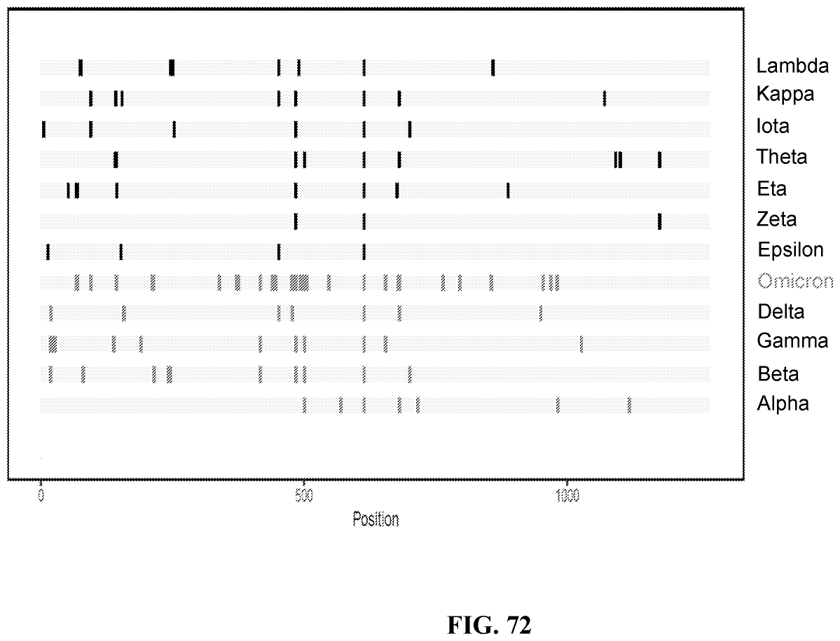

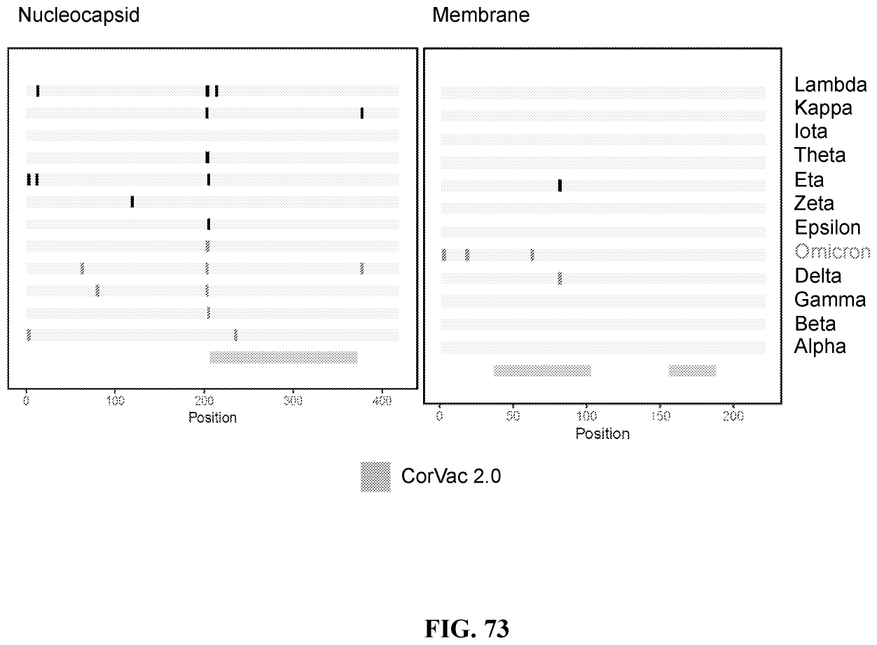

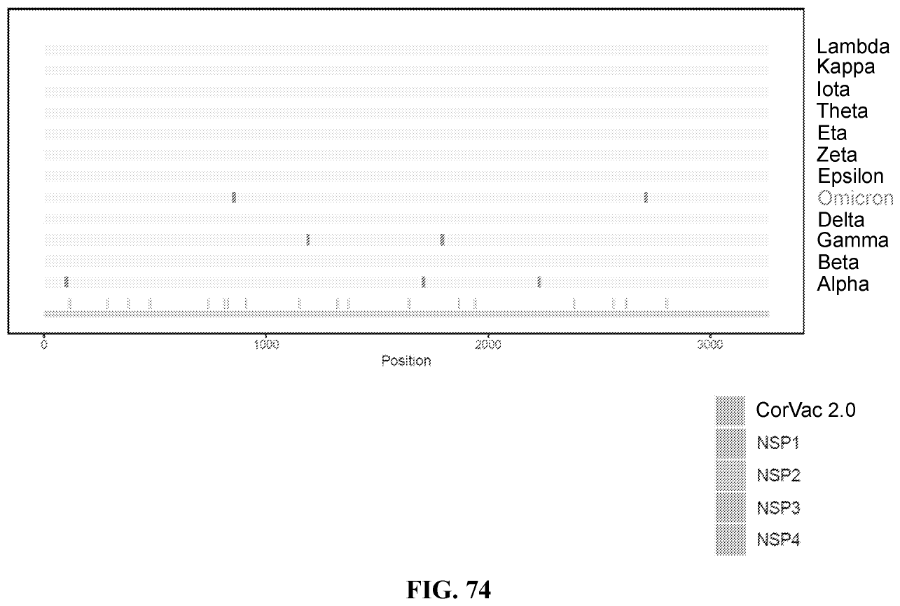

CorVac 2.0 is a T cell targeting vaccine that can complement and enhance BNT162b2. It can increase stability of the vaccine in the face of emerging SAS-CoV-2 variants including the delta and omicron variants (see FIGS. 72-74), leverage CD4 and CD8 T cell immunity against SARS-CoV-2 to protect afainst severe CoVID 19 in one or more of the following ways, enhance and broaden T cell response, shorten clearance of the virus, provide persistent long-term memory to T cell responses, and potentially enhance B cell response and durability through increased CD4 activation. An important use of CorVac 2.0 is that the vaccine can be protective over a broad range of variants and can overcome variants that could reduce vaccine efficacy. Moreover, as shown in FIGS. 73 and 74, CorVac2.0 sequence is rarely impacted by SAS-CoV-2 variant mutations With the encouraging animal studies described herein, a clinical program is designed on healthy and immunocompromised cohort.

Healthy subjects cohort: This study participants will receive a combination of CorVac2.0 with BNT162b2 as a booster to: enhance and broaden T cell responses beyond the spike T cell responses from BNT162b2 and potentially shorten time to viral clearance, increase stability in the face of emerging SARS-CoV-2 variants; provide persistent long-term memory of T cell responses. In one aspect, vaccine naïve cohorts could be explored for combination or monotherapy.

A target product profile could be tested for the following quailities. In a prospective study, (i) one group will receive BNT162b2 and CorVac 2.0 (any variant); (ii) a second group will receive CorVac 2.0 monovaccine as a booster after BNT162b2 vaccinations. For the first group, the product composition will be nucleoside modified RNA encoding spans of the nucleocapsid and membrane proteins and short CD8+targeting epitopes of ORF1ab co-administered with BNT162b2. For the second group, the product composition will be nucleoside modified RNA encoding spans of the nucleocapsid and membrane proteins and short CD8+targeting epitopes of ORF1ab.

Indication: Prevention of CoVID-19 in adults and children.

Target population for both grops in this study will include adults 18 years or older, children 6 months of age and above, pregnant women. The immunogenicity relate outcome to be tested will include significant SARS-CoV-2 neutralizing response, antigen-specific CD4+ T cell response, and antigen-specific IFNγ producing CD8+ T cell response, protection against variants of SARS-CoV-2.

Efficacy of the product: The study will determine if there is (i) clear demonstration of efficacy or observational effectiveness with ≥70% point estimate (lower bound of confidence interval >30%) (ii) Prevention of severe disease (e.g.,, hospitalization, mechanical ventilation, death) in any of the groups. Onset of immunity will be tested within 2 weeks of booster dose. Duration of protection will be tested.

Dosing schedule and route of administration: intramuscular (IM) injection of 30 microgram/dose total of the product (BNT162b2 and CorVac 2.0 alone or BNT162b2+CorVac 2.0). 2 injections would be administered at an interval of 21 days, and a booster dose 6 months after. Storage can be at 2-8° C. for at least 3 months.

Drug safety: Increased reactogenicity acceptable, such as local injection site and systemic side effects (if balanced with clinical benefit) and can be administered with other vaccine (e.g., flu vaccine). Table 17 lists some of the above.

TABLE 17
Target Product Profile-SARS-COV-2 T cell targeting vaccine
(CorVac2.0) for healthy vaccinated subjects

| | Co-admiinstration of CorVac 2.0 | CorVac 2.0 mono-vaccine |
|---|---|---|
| Product Attribute | with BNT162b2 (or variant) | booster post BNT162b2 |
| Indication | For the prevention of COVID-19 | For the prevention of COVID-19 |
| | for adults and children | for adults and children |
| Product Composition | Nucleoside modified RNA | Nucleoside modified RNA |
| | encoding spans of the nucleocapsid | encoding spans of the nucleocapsid |
| | and membrane proteins and short | and membrane proteins and short |
| | CD8+ targeting epitopes of ORF1ab | CD8+ targeting epitopes of |
| | co-administered with BNT162b2 | ORF1ab |
| Target Population | Adults 18 years and above, children | Adults 18 years and above, |
| | 6 months of age and above, | children 6 months of age and |
| | pregnant women | above, pregnant women |
| Immunogenicity | Significant SARS-COV-2 | Significant SARS-COV-2 |
| | neutralizing response, antigen- | neutralizing response, antigen- |
| | specific CD4+T cell response, and | specific CD4+T cell response, and |
| | antigen-specific IFNγ producing | antigen-specific IFNγ producing |
| | CD8+T cell response, protection | CD8+T cell response, protection |
| | against variants of SARS-COV-2 | against variants of SARS-COV-2 |
| Efficacy | Clear demonstration of efficacy or | Clear demonstration of efficacy or |
| | observational effectiveness with | observational effectiveness with |
| | ≥70% point estimate (lower bound | ≥70% point estimate (lower bound |
| | of confidence interval >30%) | of confidence interval >30%) |
| | Prevention of severe disease (e.g.., | Prevention of severe disease (e.g.., |
| | hospitalization, mechanical | hospitalization, mechanical |
| | ventilation, death) | ventilation, death) |
| Onset of Immunity | Within 2 weeks of booster dose | Within 2 weeks of booster dose |
| Duration of Protection | Confers protection for at least 1 | Confers protection for at least 1 |
| | year, including T cell protection | year, including T cell protection |
| | against variants of SARS-COV-2 | against variants of SARS-COV-2 |
| Safety and Drug | Confers protection for at least 1 | Confers protection for at least 1 |
| Interaction | year, including T cell protection | year, including T cell protection |
| | against variants of SARS-COV-2 | against variants of SARS-COV-2 |
| Dosing Schedule/Route | IM injection, 30 μg/dose total | IM injection, 30 μg/dose total |
| | 2 injections separated by 21days, | 2 injections separated by 21days, |
| | booster after 6 months | booster after 6 months |
| of Administration | Supplied as SDV and preserved | Supplied as SDV and preserved |
| | MDV | MDV |
| | Storage at 2-8° C. for at least | Storage at 2-8° C. for at least |
| | 3 months | 3 months |

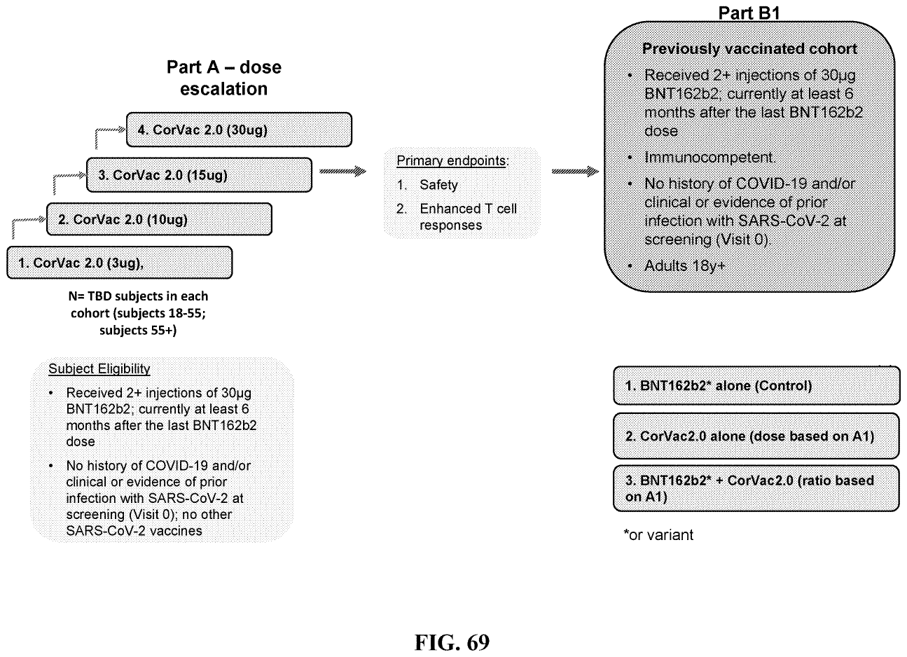

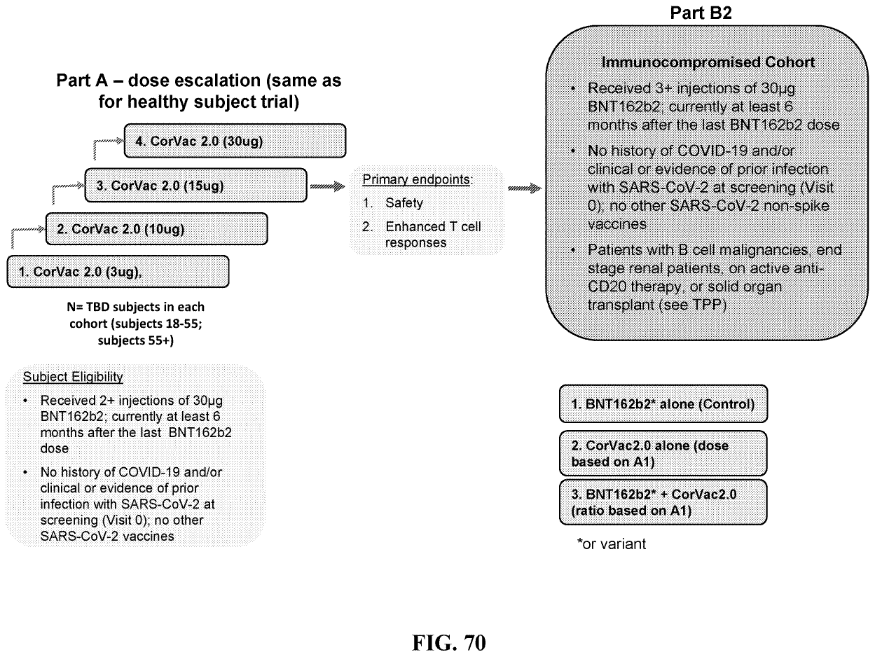

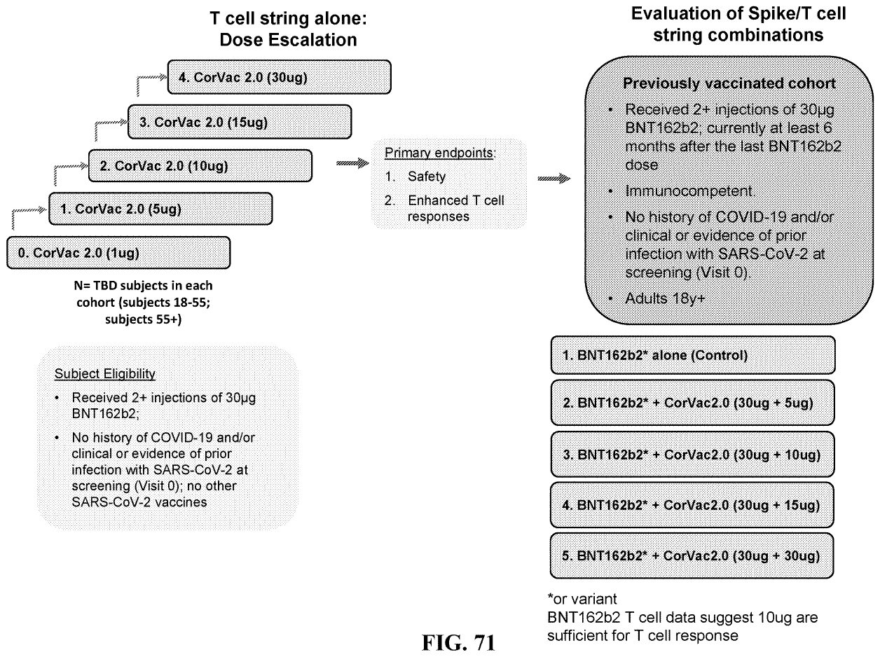

A proposed Phase 1 trial is outlined in FIG. 69. Part A of the study will be dose escalation and safety evaluation of CorVac 2.0, starting at 3 micrograms, escalating to 10 μg, 15 μg and 30 μg. Subjects may be 18 to 55 years of age, and, may be older than 55 years. Eligibility criteria for inclusion in the study would be that the subjects will have received 2 or more injections of the 30 μg dose of BNT162b2, and 6 months will have elapsed from the last BNT162b2 dose. The subject should have no history of COVID 19 or like and/or no evidence of prior infection with SARS-CoV2 at screening visit (Visit 0). The subject should have been administered no other SARS-CoV-2 vaccine in the past. Based on the dose escalation study's safety evaluation, a Go/No go decision will be established, and Part B will commence. For this, the inclusion criteria include that the subjects will have received 2+injections of 30 ug BNT162b2; currently at least 6 months after the last BNT162b2 dose; should be immunocompetent. With no history of COVID-19 and/or clinical or evidence of prior infection with SARS-CoV-2 at screening (Visit 0). Participant should be adults 18y+. The study will have three groups, one group will be administered BNT162b2 alone, the second group will be administered CorVac 2.0 alone, based on study results of Part A. The third group will receive a combination vaccine, BNT162b2+CorVac 2.0, ratio based on A1.

### Immunocomprised Patients:

CorVac 2.0 can provide broad T cell immunity to patient cohorts that have compromised B cell immunity, such as solid organ transplant subjects, patients with hematological malignancies, or patients receiving B cell targeting therapies. A target product profile can be tested for the following quailities. In a prospective CorVac2.0, a SARS-CoV-2 T cell-based vaccine could be tested in immunosuppressed patient subjects. These subjects can be divided into 2 groups, (i) in one group, a monovaccine will be administered and (ii) in the second group. A CorVac 2.0 with BNT162b2 (any variant) will be coadministered.

Indications: For the each group, the indications would be prevention of severe COVID19 and hospitalization in immunocompromised patients who are 18 years of age or older who have received 3 doses of BNT162b2. The product to be administered will be nucleoside modified RNA encoding spans of the nucleocapsid and membrane proteins and short CD8+targeting epitopes of ORF1ab for group 1, and nucleoside modified RNA encoding spans of the nucleocapsid and membrane proteins and short CD8+targeting epitopes of ORF1ab would be delivered with separately formulated BNT162b2.

The target population for each group would be (a) Patients with B cell malignancies who are either asymptomic, anti-CD20 monotherapy treatments or targeted drug therapy, (b) End stage renal patients (Maintenance hemodialysis treatment secondary to end-stage renal disease and is ≥18 years of age) (c) Subjects, who are on active immunomodulator therapy targeting anti-CD20 for an autoimmune inflammatory disorder (eg, inflammatory arthritis, such as rheumatoid arthritis, psoriatic arthritis, and juvenile idiopathic arthritis, and inflammatory bowel disease, such as ulcerative colitis and Crohn's disease) at a stable dose, (d) Subjects with solid organ transplant (receiving a solid organ transplant at least 3 months (84 days) prior to enrollment (Visit 1) and with no acute rejection episodes within 2 months (60 days) prior to enrollment (Visit 1)).

Immunogenicity will be tested to show if there was antigen-specific TH1-phenotype CD4+ T cell response, and antigen-specific IFNγ producing CD8+ T cell response in group (i) subjects, and if comparable SARS-CoV-2 Spike neutralizing response, antigen-specific TH1-phenotype CD4+ T cell response, and antigen-specific IFNγ producing CD8+ T cell response in group (ii) subjects.

Efficacy of the product: The study will determine if there is (i) clear demonstration of efficacy or observational effectiveness with ≥50* % point estimate (lower bound of confidence interval >30%) in any of the groups, and also (ii) prevention of severe disease (e.g., hospitalization, mechanical ventilation, death). Onset of immunity will be tested within 2 weeks of vaccine dose. Duration of protection will be tested.

Drug safety: Increased reactogenicity acceptable, such as local injection site and systemic side effects (if balanced with clinical benefit) and can be administered with other vaccine (e.g., flu vaccine). For group (i), there will be no combination with other vaccines; for group (ii) there will be coadministration of BNT162b2.

Dosing schedule and route of administration: intramuscular (IM) injection of 30 microgram/dose total of the product (BNT162b2 and CorVac 2.0 alone or BNT162b2+CorVac 2.0). 2 injections would be administered at an interval of 21 days, and a booster dose 6 months after. Storage can be at 2-8° C. for at least 3 months.

Table 18 lists some of the above.

TABLE 18
Target Product Profile-SARS-COV-2 T cell-based
vaccine in immunosuppressed patients

| | | Co-adminstration CorVac2.0 |
|---|---|---|
| Product Attribute | Mono-vaccine | with BNT162b2 (or variant) |
| Indication | For the prevention of severe | For the prevention of severe |
| | COVID19 and hospitalization in | COVID19 and hospitalization in |
| | immunocompromised patients 18 | immunocompromised patients 18 |
| | years of age or older who have | years of age or older 3 doses of |
| | received 3 doses of BNT162b2 | BNT162b2 |
| Product Composition | Nucleoside modified RNA | Nucleoside modified RNA |
| | encoding spans of the nucleocapsid | encoding spans of the nucleocapsid |
| | and membrane proteins and short | and membrane proteins and short |
| | CD8+ targeting epitopes of ORF1ab | CD8+ targeting epitopes of |
| | | ORF1ab delivered with separately |
| | | formulated BNT162b2 |
| Target Population | Patients with B cell malignancies | Patients with B cell malignancies |
| | who are either asymptomic, anti- | who are either asymptomic, anti- |
| | CD20 monotherapy treatments or | CD20 monotherapy treatments or |
| | targeted drug therapy | targeted drug therapy |
| | End stage renal patients | End stage renal patients |
| | (Maintenance hemodialysis | (Maintenance hemodialysis |
| | treatment secondary to end-stage | treatment secondary to end-stage |
| | renal disease and is | renal disease and is |
| | ≥18 years of age) | ≥18 years of age) |
| | Is on active immunomodulator | Is on active immunomodulator |
| | therapy targeting anti-CD20 for an | therapy targeting anti-CD20 for an |
| | autoimmune inflammatory disorder | autoimmune inflammatory |
| | (eg, inflammatory arthritis, such as | disorder (eg, inflammatory |
| | rheumatoid arthritis, psoriatic | arthritis, such as rheumatoid |
| | arthritis, and juvenile idiopathic | arthritis, psoriatic arthritis, and |
| | arthritis, and inflammatory bowel | juvenile idiopathic arthritis, and |
| | disease, such as ulcerative colitis | inflammatory bowel disease, such |
| | and Crohn's disease) at a stable dose | as ulcerative colitis and Crohn's |
| | Solid organ transplant (Receiving a | disease) at a stable dose |
| | solid organ transplant at least 3 | Solid organ transplant (Receiving a |
| | months (84 days) prior to | solid organ transplant at least 3 |
| | enrollment (Visit 1) and with no | months (84 days) prior to |
| | acute rejection epi-sodes within 2 | enrollment (Visit 1) and with no |
| | months (60 days) prior to | acute rejection epi-sodes within 2 |
| | enrollment (Visit 1)) | months (60 days) prior to |
| | | enrollment (Visit 1)) |
| Immunogenicity | Antigen-specific TH1-phenotype | Comparable SARS-COV-2 Spike |
| | CD4+T cell response, and antigen- | neutralizing response, antigen- |
| | specific IFNγ producing CD8+T | specific TH1-phenotype CD4+T |
| | cell response Comparable SARS- | cell response, and antigen-specific |
| | CoV-2 Spike neutralizing response, | IFNγ producing CD8+T cell |
| | antigen-specific TH1-phenotype | response2 |
| | CD4+T cell response, and antigen- | |
| | specificIFNγ producing CD8+T | |
| | cell response | |
| Efficacy | Clear demonstration of efficacy or | Clear demonstration of efficacy or |
| | observational effectiveness with | observational effectiveness with |
| | ≥50*% point estimate (lower bound | ≥50*% point estimate (lower |
| | of confidence interval >30%)* | bound of confidence interval >30%)* |
| | | Prevention of severe disease (e.g.., |
| | | hospitalization, mechanical |
| | | ventilation, death) |
| Onset of Immunity | Within 2 weeks of vaccine dose | Within 2 weeks of vaccine dose |
| Duration of Protection | Confers protection from severe | Confers protection for at least 6 |
| | disease for at least 6 months through | months; provides protection of |
| | T cell responses | severe disease against variants of |
| | | concern |
| Safety and Drug | Increased reactogenicity | Increased reactogenicity |
| Interaction | acceptable, such as local injection | acceptable, such as local injection |
| | site and systemic side effects (if | site and systemic side effects (if |
| | balanced with clinical benefit) | balanced with clinical benefit) |
| | No combination with other vaccines | Co-administration with BNT162b2 |
| Dosing Schedule/ | IM injection, 30 μg/dose | IM injection, 30 μg dose (total |
| Route of | 2 injections separated by 21days | dose of both BNT162b2 + |
| Administration | Supplied as SDV and preserved MDV | CorVac 2.0) |
| | Storage at 2-8° C. for at least | 2 injections separated by 21days |
| | 3months | Supplied as SDV and preserved MDV |
| | | Storage at 2-8° C. for at least 3 |
| | | months |

A proposed Phase 1 trial for clinical evaluation of CorVac 2.0 in immunocompromised patients is outlined in FIG. 70. Part A of the study will be dose escalation and safety evaluation of CorVac 2.0, starting at 3 micrograms, escalating to 10 μg, 15 μg and 30 μg. Subjects may be 18 to 55 years of age, and, may be older than 55 years. Eligibility criteria for inclusion in the study would be that the subjects will have received 2 or more injections of the 30 μg dose of BNT162b2, and 6 months will have elapsed from the last BNT162b2 dose. The subject should have no history of COVID 19 or like and/or no evidence of prior infection with SARS-CoV2 at screening visit (Visit 0). The subject should have been administered no other SARS-CoV-2 vaccine in the past. Based on the dose escalation study's safety evaluation, a Go/No go decision will be established, and Part B will commence on immunocompromised cohort. For this, the inclusion criteria include that the subjects will have received 3+injections of 30 μg BNT162b2; currently at least 6 months after the last BNT162b2 dose, with no history of COVID-19 and/or clinical or evidence of prior infection with SARS-CoV-2 at screening (Visit 0). Participant will not have received any other spike or non-spike vaccine in the past. Participant should be adults 18y+. Patients with B cell malignancies, end stage renal patients, on active anti-CD20 therapy, or solid organ transplant (see TPP) will be included. The study will have three groups, one group will be administered BNT162b2 alone, the second group will be administered CorVac 2.0 alone, based on study results of Part A. The third group will receive a combination vaccine, BNT162b2+CorVac 2.0, ratio based on A1.

### Example 25. MS Validation of C7 T-String

To examine whether the C7 T-string can generate T cell epitopes that are processed and presented onto HLA complexes, targeted mass spectrometry was used to detect peptides from the C7 T-string bound to HLA-I molecules. To accurately determine which HLA allele an epitope is bound to, monoallelic profiling of tagged allele constructs (MAPTAC) was used to isolate a single allele from the endogenous alleles present in the A375 cell line (in the present example, a protocol similar to that used in Abelin, Jennifer G., et al. “Defining HLA-II ligand processing and binding rules with mass spectrometry enhances cancer epitope prediction.” Immunity 51.4 (2019): 766-779; and Choi, Jaewon, et al. “Systematic discovery and validation of T cell targets directed against oncogenic KRAS mutations.” Cell reports methods 1.5 (2021): 100084 was used, the contents of each of which is incorporated by reference herein in their entirety).

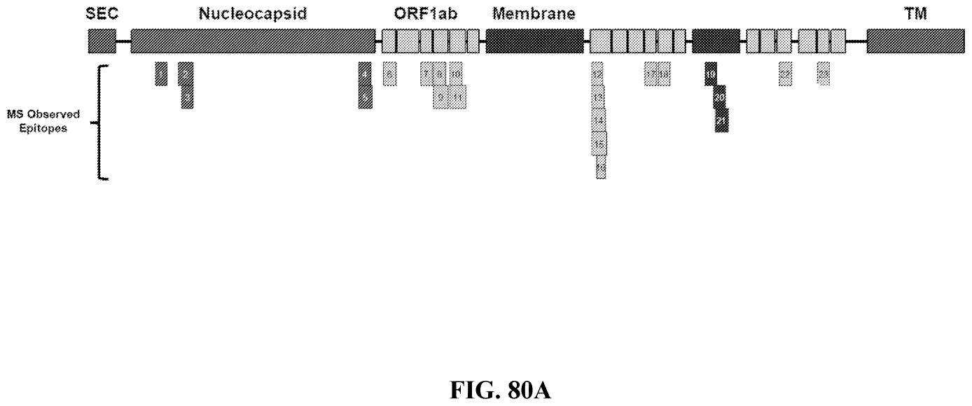

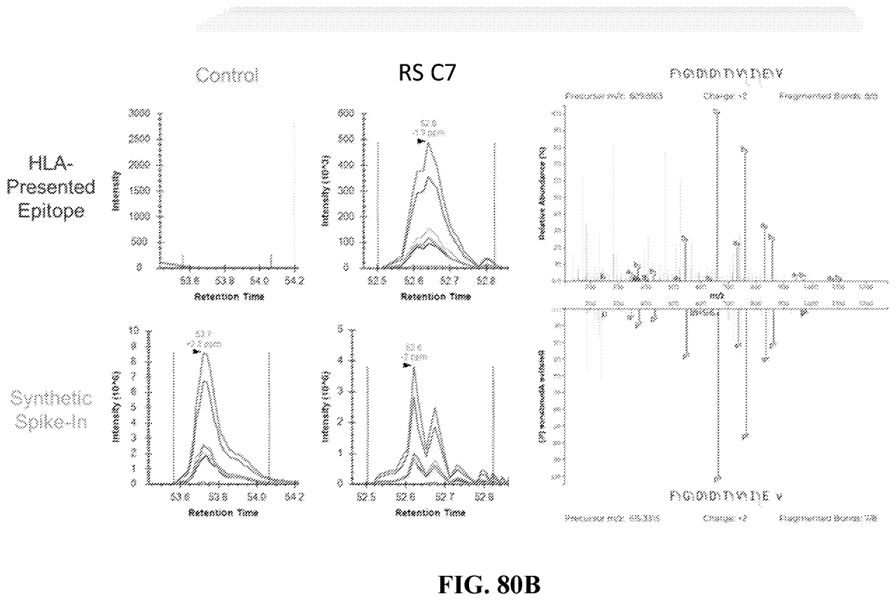

In addition, the pan-HLA-I antibody W6/32 was used to profile C7 T-string epitopes bound to alleles endogenous to the cell line used. Altogether, 20 unique peptide epitopes were detected across seven HLA alleles with 22 unique peptide-HLA pairs observed (FIG. 80A). Verified peptides spanned the entire length of the C7 T-string and peptides derived from all encoded viral antigens were detected. Notably, of the 20 MS-verified epitopes, 15 were cited as immunogenic in the literature, with 8 of them being cited as “immunodominant” within the respective studies (Ferretti et al., 2020; Francis et al., 2022; Gangaev et al., 2020; Kared et al., 2021; Saini et al., 2021). Table 19, below, lists the epitopes observed in these experiments.

TABLE 19
Observed T-cell Epitopes Encoded
by the C7 T-string Construct

| # | Epitope | Allele(s) |
|---|---|---|
| 1 | LLLDRLNQL | A02:01 |
| 2 | GQQQQGQTVTK | A11:01 |
| 3 | QQQGQTVTK | A11:01 |
| 4 | KTFPPTEPK | A11:01, A03:01 |
| 5 | KTFPPTEPKK | A11:01, A03:01 |
| 6 | VTNNTFTLK | A11:01 |
| 7 | KTIQPRVEK | A11:01, A03:01 |
| 8 | YLFDESGEF | A02:01 |
| 9 | YLFDESGEFKL | A02:01 |
| 10 | SEVGPEHSL | B44:03* |
| 11 | SEVGPEHSLAEY | B44:03* |
| 12 | TTDPSFLG | A01:01 |
| 13 | TTDPSFLGR | A01:01 |
| 14 | TTDPSFLGRY | A01:01, A02:01, A03:01, |
| | | A11:01, B08:01 |
| 15 | TTDPSFLGRYM | A01:01 |
| 16 | PSFLGRY | A01:01 |
| 17 | QLMCQPILL | A02:01 |
| 18 | AEAELAKNV | B44:03* |
| 19 | LPKEITVAT | B08:01 |
| 20 | VATSRTLSY | C16:01* |
| 21 | ATSRTLSYYK | A11:01 |
| 22 | PTDNYITTY | A01:01, A02:01, A03:01, |
| | | A11:01, B08:01 |
| 23 | FGDDTVIEV | A02:01 |

*Allele inferred from score in W6/32 pulldown.

### Example 26. Further Immunogenicity Studies of CorVac 2.0 C-7 String with BNT162b2 in k18-hACE2 Mice

In the present Example, different dosing regimens strategies were compared in k18-hACE2 mice to test the impact of CorVac 2.0 on anti-Spike antibodies induced by a coronavirus vaccine (e.g., BNT162b2) when administered at different ratios, where the coronavirus vaccine (e.g., BNT162b2) dosage was kept constant so as to not reduce Ab titers.

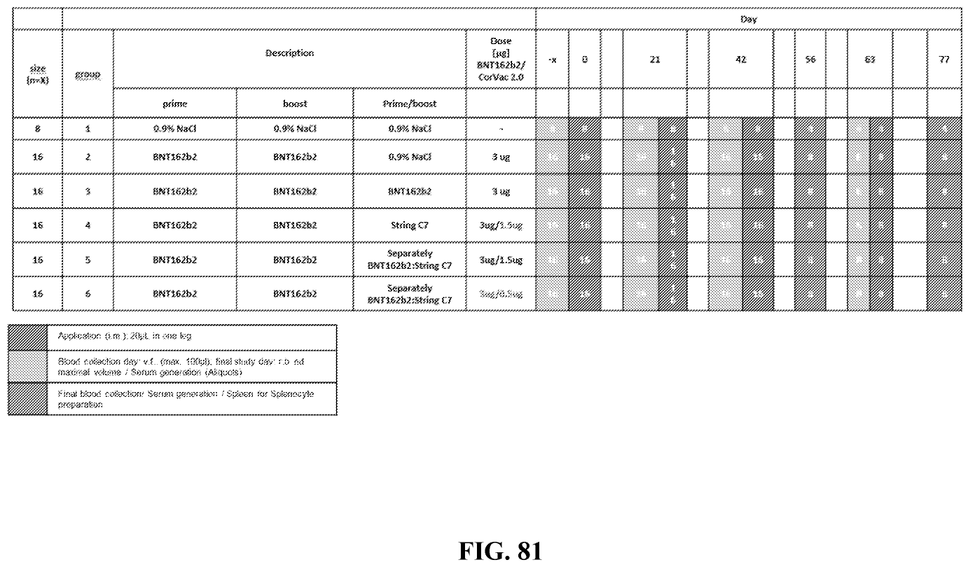

The specific dosing regimens tested in the present Example are summarized in FIG. 81, and include (a) two doses of 3 micrograms of a spike mRNA vaccine alone (in the present Example BNT162b2), (b) four doses of 3 micrograms of a spike mRNA vaccine alone, (c) two 3 microgram doses of a spike mRNA vaccine alone followed by two 1.5 microgram doses of CorVac2.0 alone (in the present example, a C7 string), (d) two 3 microgram doses of a spike mRNA vaccine alone followed by a third and fourth dose of separately formulated CorVac2.0 and a spike mRNA vaccine, where the third and four dose comprise 3 micrograms of a spike mRNA vaccine and 1.5 micrograms of CorVac2.0 RNA, and (e) two 3 microgram doses of a spike mRNA vaccine alone followed by a third and fourth dose of separately formulated CorVac2.0 and a spike mRNA vaccine, where the third and four dose comprise 3 micrograms of a spike mRNA vaccine and 0.5 micrograms of CorVac2.0 RNA.

Compositions were administered on days 0, 21, 42, and 63 (where day 0 corresponds to administration of the first dose). Blood samples were collected on days 0 (before first dose), 21, 42, 56 and 63 and analyzed for concentrations of antibodies that bind the SARS-CoV-2 Spike protein and neutralization titers. On days 56 and 77, half of the animals in each group were sacrificed, and spleens were harvested for anlysis.

### ELISA

Serum samples were tested in 96-well plates for antigen specific antibody concentrations (in the present Example, concentrations of anti-Spike protein antibody cocentrations). Each well of a MaxiSorp plate was coated with 100 ng/100 μL recombinant Spike protein per well or isotype controls according to plate layout. Plates were incubated at 4° C. o/n. Wells were washed and blocked with 1x blocking buffer and incubated at 37° C. for 1 h on a shaker. Plates were washed and dilutions of the positive control antibody/serum samples were added and incubated at 37° C. for 1 h on a shaker. Plates were washed three times with 300 μL/well PBS-T and the diluted secondary antibodies were incubated at 37° C. for 45 min on shaker. After a wash, TMB substrate was added and incubated for 8 min at RT. The reaction was stopped with 100 μL 25% sulfuric acid and read on plate reader (450 nm, reference: 620 nm).

For concentration analysis, the signal of the specific samples was correlated to a positive control antibody. For reciprocal serum endpoint titer, the serum dilution that emitted the OD exceeding 4-fold background was used. The background was defined as the OD signal given by the recombinant protein incubated with the secondary detection anti-mouse IgG antibody only.

### Pseudovirus-Based Neutralization Assay

Replication-deficient vesicular stomatitis virus (VSV) that lacks the genetic information for the VSV envelope glycoprotein VSV-G but contains an open reading frame (ORF) for green fluorescent protein (GFP) was used for SARS-CoV-2—S pseudovirus generation. VSV pseudotypes were generated according to a published protocol (Hoffmann M, Kleine-Weber H, et al. SARS-CoV-2 Cell Entry Depends on ACE2 andTMPRSS2 and Is Blocked by a Clinically Proven Protease Inhibitor Cell. 2020; 181 (2): 271-280, the contents of which are incorporated by reference herein in their entirety). In brief, HEK293T/17 cells cultured in DMEM supplemented with 10% FBS were transfected with a pcDNA3.1-derived expression plasmid (Invitrogen) coding for the SARS-CoV-2 spike protein (GenBank ID: QHD43416.1) with shortened cytoplasmic tail, i.e., pSARS-CoV-2-S-CΔ19, using Lipofectamine® LTX & PLUS™ Reagent (Invitrogen) following the manufacturer's instructions. The cytoplasmic tail was truncated for the 19 C-terminal amino acids to facilitate a more efficient integration of SARS-CoV-2-S into VSV virions analogous to SARS-COV-S pseudotyped VSV (Fukushi et al. 2005). At 24 h post transfection, cells were inoculated with VSV-G Trans-complemented VSV-ΔG-GFP vector (Indiana strain, de novo generated by reverse genetics from plasmid (Lawson et al. 1995)) at a multiplicity of infection (MOI) of three and incubated for 2 h at 37° C. and 5% CO2. Next, the inoculum was removed, cells were washed with PBS, and standard culture medium which contained 0.5 μg/mL anti-VSV-G antibody (clone 8G5F11) was added to neutralize residual input virus. Twenty-four hours after infection, VSV/SARS-CoV-2—S pseudovirus-containing supernatants were harvested, filtered (0.45 μm) and stored at −80° C. in aliquots until further use.

For titration of VSV/SARS-CoV-2—S pseudovirus, Vero-76 cells (ATCC) were thawed diluted to 2.67×10⁵ cells/mL in assay medium (DMEM and 10% FBS) and seeded in 96-well flat-bottom plates at 4×10⁴ cells per well. Cells were incubated for 4 to 6 h at 37° C. and 7.5% CO2. Meanwhile, two-fold, eight-step serial dilutions were prepared in 96-well V-bottom plates beginning with undiluted pseudovirus supernatant. Vero-76 wells were inoculated with 50 μL of the diluted pseudovirus supernatant and incubated for 16 to 24 h at 37° C. and 7.5% CO2. Each dilution was tested in duplicate wells. After the incubation, the cell culture plates were removed from the incubator, placed in an IncuCyte Live Cell Analysis system (Essen Bioscience) and equilibrated for 30 min prior to the analysis. Whole well scanning for brightfield and GFP fluorescence was performed using a 4×objective. The number of infected GFP-fluorescent cells per well was plotted as a function of pseudovirus supernatant dilution using GraphPad Prism. Data (x=logx) were fitted with linear regression and the derived slope and γ-intercept used to calculate the amount of viral supernatant needed to obtain 144 infected cells/96-well (20% excess for virus neutralization test included).

For the pVNT assay, Vero-76 cells were thawed, diluted to 2.67×10⁵ cells/mL in assay medium (DMEM and 10% FBS) and seeded in 96-well flat-bottom plates at 4×10⁴ cells per well. Cells were incubated for 4 to 6 h at 37° C. and 7.5% CO2. Initial dilutions of mouse serum samples were prepared by adding 10 μL of serum to 50 μL assay medium in a 96-well V-bottom plate. Seven additional dilutions were subsequently prepared in two-fold dilution steps, by iteratively transferring 30 μL of diluted sera to wells containing 30 μL assay medium. VSV/SARS-CoV-2 pseudovirus was thawed and diluted to obtain 120 infected cells/25 μL (4.8×10³ infectious units [IU]/mL). 30 μL of diluted pseudovirus (corresponds to 144 infected cells) were added to the wells containing the serum dilution series. Pseudovirus/serum dilution mix was incubated for 5 min at RT on a microplate shaker at 750 rpm, and additional 5 min at RT without agitation. Pseudovirus/serum dilution mix was then added to the seeded Vero-76 cells (50 μL mix per well, MOI: 0.003), followed by incubation for 16 to 24 h at 37° C. and 5% CO2. Each dilution of serum samples was tested in duplicate wells. Vero-76 cells incubated with pseudovirus in the absence of mouse sera were used as positive controls. Vero-76 cells incubated without pseudovirus were used as negative controls. After the incubation, the cell culture plates were removed from the incubator, placed in an IncuCyte Live Cell Analysis system and incubated for 30 min prior to the analysis. Whole well scanning for brightfield and GFP fluorescence was performed using a 4×objective. To calculate the neutralizing titer, infected GFP-positive cell number per well was compared with the no-serum pseudovirus positive control. Mean values of the no-serum pseudovirus positive control multiplied by 0.5 represent the pseudovirus neutralization 50% (pVN50); mean values of the no-serum pseudovirus positive control multiplied by 0.1 represent the pseudovirus neutralization 90%. Serum samples with mean values below this cut-off exhibit >50% or >90% virus neutralization activity, respectively.

### Results

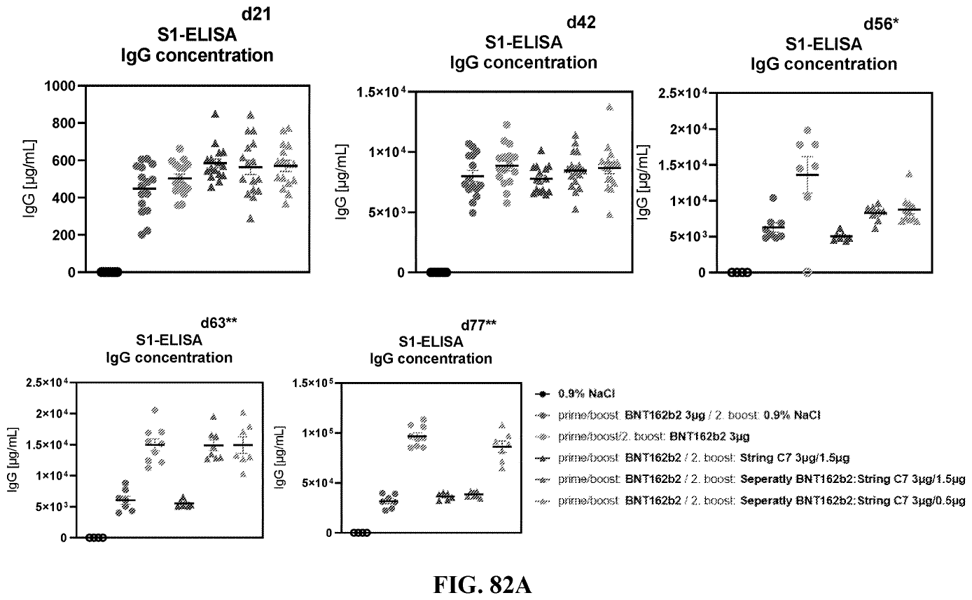

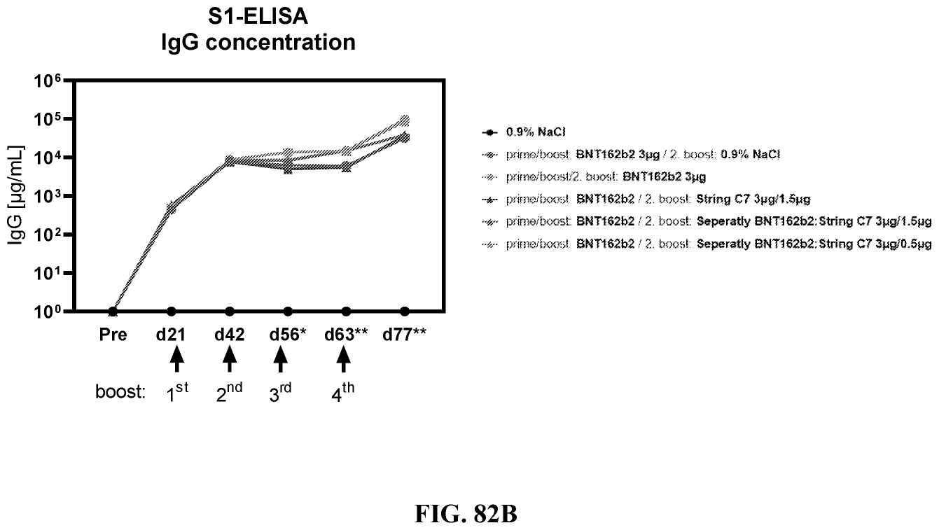

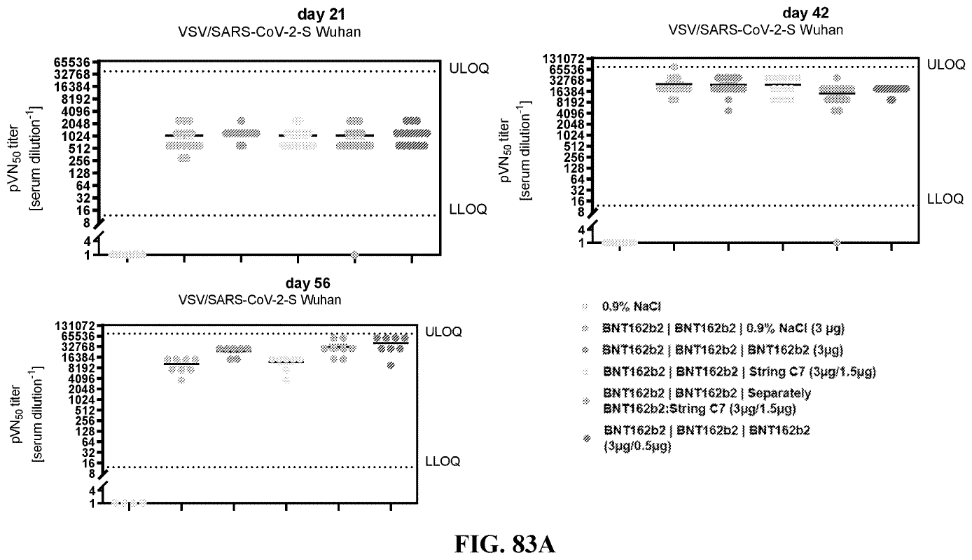

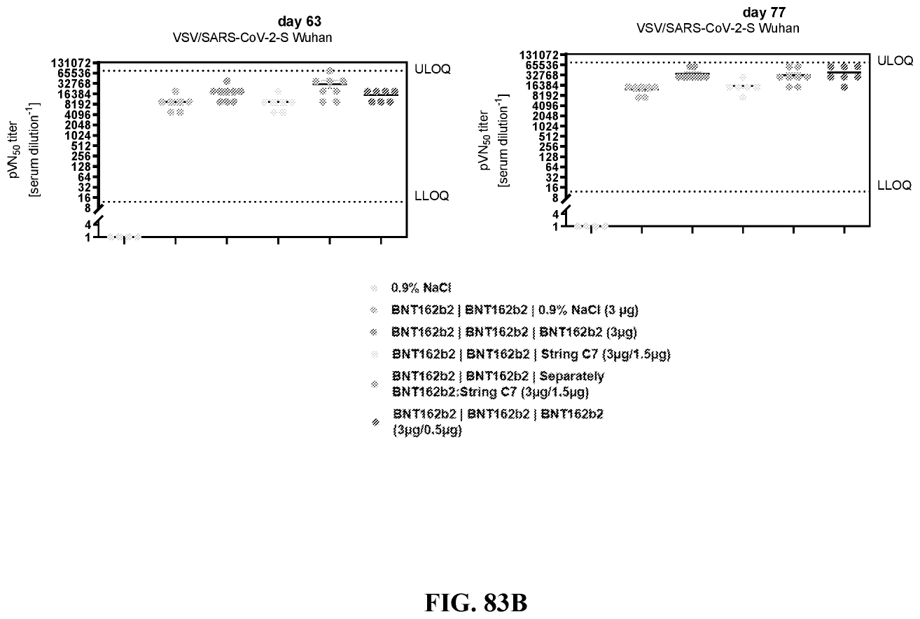

As shown in FIGS. 82A-82B, animals that had been given two doses of a spike specific mRNA followed by two doses of CorVac2.0 showed similar Spike-specific IgG concentrations as animals that had been given only two doses of a spike specific mRNA. Animals administered four doses of a spike specific mRNA also showed similar Spike-specific antibody levels as animals that had been given two doses of a spike specific mRNA followed by two doses of the combination of a spike specific mRNA and CorVac2.0. These IgG results indicate that administration of CorVac2.0 does not impair Spike IgG responses induced by a spike specific mRNA. Similarly, as shown in FIGS. 83A-B, administration of CorVac2.0 was also not found to negatively impact neutralization titers induced by an mRNA encoding an S protein.

### Example 27: Efficacy Studies in Syrian Hamsters

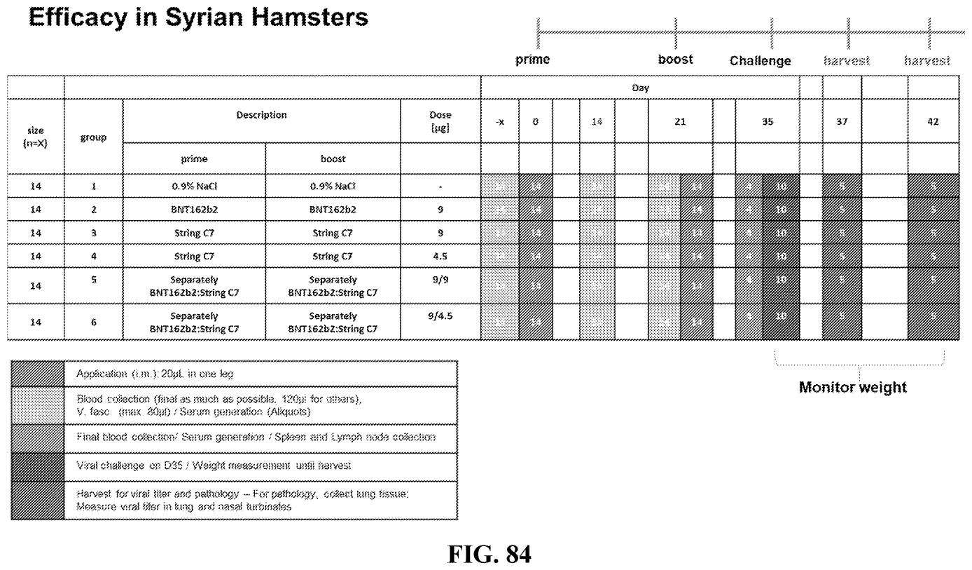

An efficacy study was performed in Syrian Hamsters to test the efficacy of compositions and dosing regimens disclosed herein. Golden Syrian hamsters were immunized with a prime dose and boost dose of either RNA encoding a SARS-CoV-2 S protein (in the present Example, BNT162b2), CorVac2.0 (in the present Example, RNA encoding RS C7), or a combination of both. Dosing regimens and sample collection protocol are summarized in FIG. 84. Hamsters were administered: (a) two 9 microgram doses of BNT162b2, (b) two 9 microgram doses of CorVac2.0, (c) two 4.5 microgram doses of CorVac2.0, (d) two doses of separately formulated BNT162b2 and CorVac2.0, where each dose comprises 9 micrograms of BNT162b2 and 9 micrgrams of CorVac2.0, and (e) two doses of separately formulated BNT162b2 and CorVac2.0, where each dose comprises 9 micrograms of BNT162b2 and 4.5 micrograms of CorVac2.0. Doses were administered on days 0 and 21, and blood samples were collected on days 0, 14, 21. Animals were challenged on D35 with the WT Wuhan virus variant. Half of the cohort was harvested two days post infection and the remain half was harvested 7 days post infection, and lungs and nasal turbinates were collected at each timepoint. Oral swabs were also taken daily to measure viral load.

SARS-CoV-2 isolate USA-WA1/2020 was used for viral challenge, and animals were challenged via intranasal inoculation (IN) on Ketamine/Xylazine anesthetized hamsters. . . . The virus was expanded in Calu-3 cells (ATCC® HTB-55) from a seed stock (BEI Resources Cat. #: NR-52281). Animals were challenged with 6×10³ pfu of USA-WA1/2020 SARS-CoV-2, with a total volume of 100 μL per animal (50 μL/nare). The infectious titers of the stock were 2.34×10⁹ TCID50/mL (3.68×10⁷ PFU/mL) in Vero TMPRSS2 cells, and 6.0×10⁵ PFU/mL in VeroE6 cells. Administration of virus inoculum was conducted as follows: Using a calibrated P200 pipettor, 50 μL of the viral inoculum was administered dropwise into each nostril. Anesthetized animals were held upright such that the nostrils of the hamsters were pointing towards the ceiling. The tip of the syringe was placed into the first nostril and virus inoculum was slowly injecting into the nasal passage, and then removed. This protocol was repeated for the second nostril. The animal's head was tilted back for about 20 seconds and then returned to its housing unit and monitored until fully recovered.

The body weight of each animal was measured starting on the day of viral infection, up to 7 days post infection. Animals immunized with CorVac2.0 showed less weight loss, as compared to animals adminsitered a saline control, in a dose-dependent manner, indicating that CorVac2.0 provides protection against SARS-CoV-2. Animals administered a combination of CorVac2.0 and RNA encoding a SARS-CoV-2 S protein showed comparable protection against infection as compared to animals administered, indicating that inclusion of CorVac2.0 in combination with BNT162b2 does not negatively impact protection, and in the group with the high dose of CorVac2.0, seems to even lead to slightly less weight loss than the BNT162b2 alone.

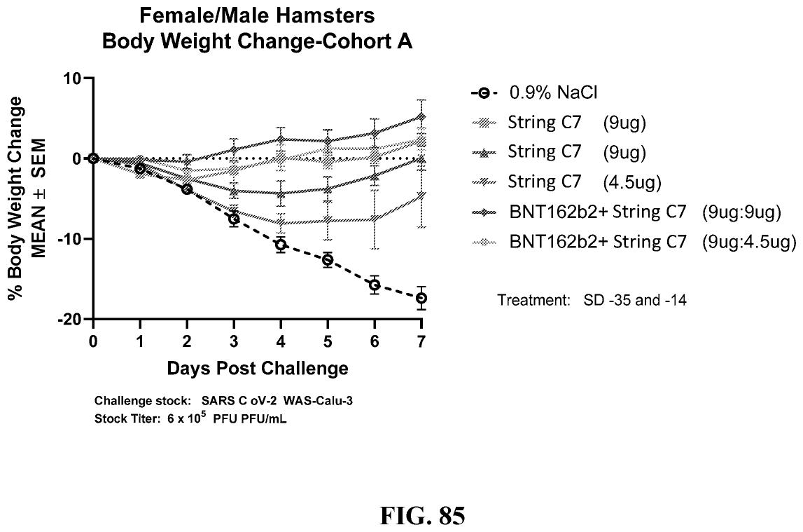

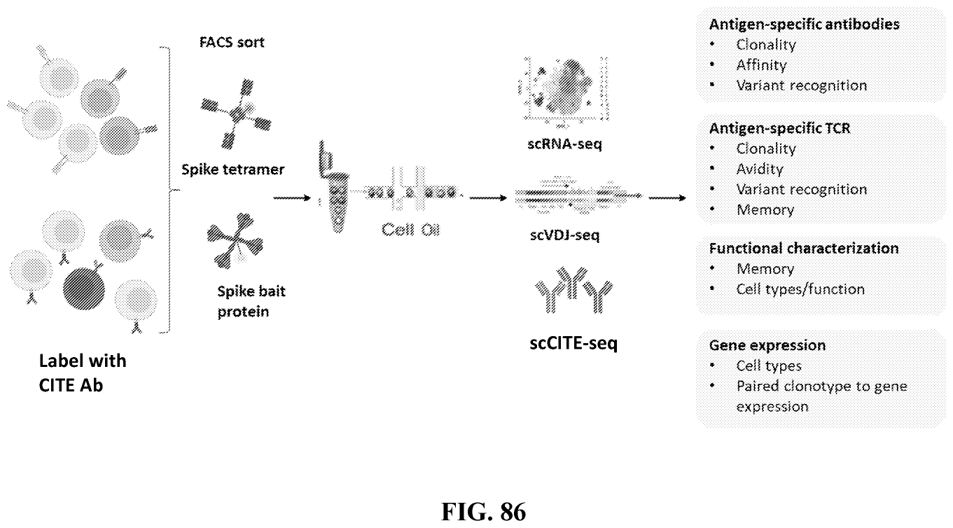

The body weight of each animal was measured starting the day of infection to 7 days post infection. As shown in FIG. 85, animals immunized with CorVac2.0 showed less weight loss as compared to a saline control, and this response was observed in a dose-dependent manner, indicating that CorVac2.0 provides protection against SARS-CoV-2. Animals administered a combination of CorVac2.0 and BNT162b2 showed no significant body weight changes as compared to animals administered BNT162b2 alone, indicating that inclusion of CorVac2.0 in combination with RNA encoding a SARS-CoV-2 S protein does not negatively impact protection against SARS-CoV-2 infection. Animals administered 9 micrograms of BNT162b2 and 9 micrograms of CorVac2.0 showed even less weight loss than animals administered 9 micrograms of BNT162b2 alone, indicating that CorVac2.0 administered in combination with an RNA encoding an SARS-CoV-2 S protein (e.g., BNT162b2) may provide improved protection as compared to RNA encoding a SARS-CoV-2 S protein alone.

### Example 28: Sequence Analysis of Spike+ T Cells and B Cells

Splenocytes were harvested from animals studied in Example 23 (specifically, animals from the groups 8 and 11 described in FIG. 66, which were administered (i) two doses of RNA encoding a SARS-CoV-2 S protein (in the present example, BNT162b2) or (ii) two doses of RNA encoding a SARS-CoV-2 S protein followed by a third dose of CorVac2.0 (in the present Example, RS C7). Spike-specific T and B cells were (a) labelled with Spike pMHC tetramers and tetramerized Spike bait protein, respectively, (b) isolated using FACS, and (c) sequenced using a 10× sequencing system to generate single cell RNA gene expression, VDJ, and CITE-seq libraries so as to understand how administration of CorVac2.0 impacts TCR and BCR clonality and phenotype. In the present example, the 10× sequencing system was used in accordance with manufactuer's recommended protocols, and VDJ and CITE-seq libraries were prepared using a protocol similar to that described in Stoeckius, Marlon, et al. “Simultaneous epitope and transcriptome measurement in single cells.” Nature methods 14.9 (2017): 865-868; and Gao, Shouguo, et al. “Single-cell RNA sequencing coupled to TCR profiling of large granular lymphocyte leukemia T cells.” Nature communications 13.1 (2022): 1-14, respectively, the contents of each of which are incorporated by reference herein in their entirety). Data was analyzed using cellranger using a protocol similar to that described in Zheng, Grace X Y, et al. “Massively parallel digital transcriptional profiling of single cells,” Nature communications 8.1 (2017): 1-12.

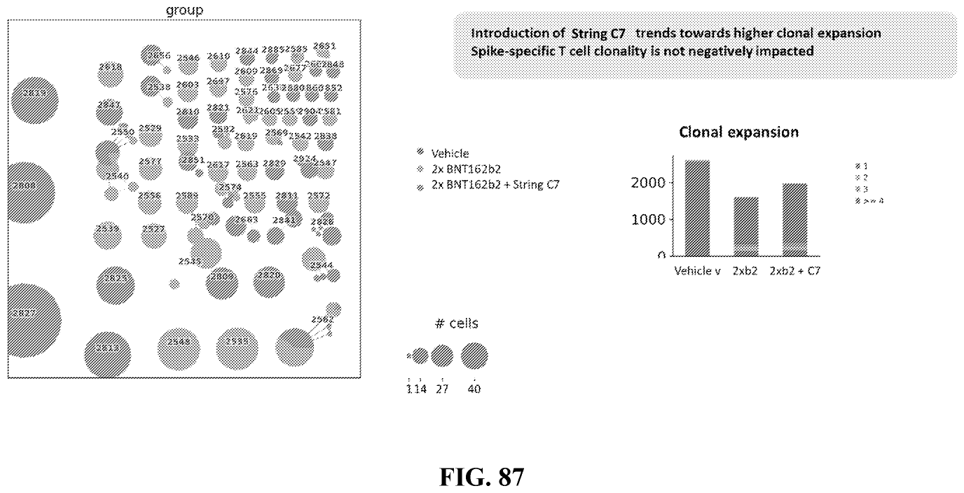

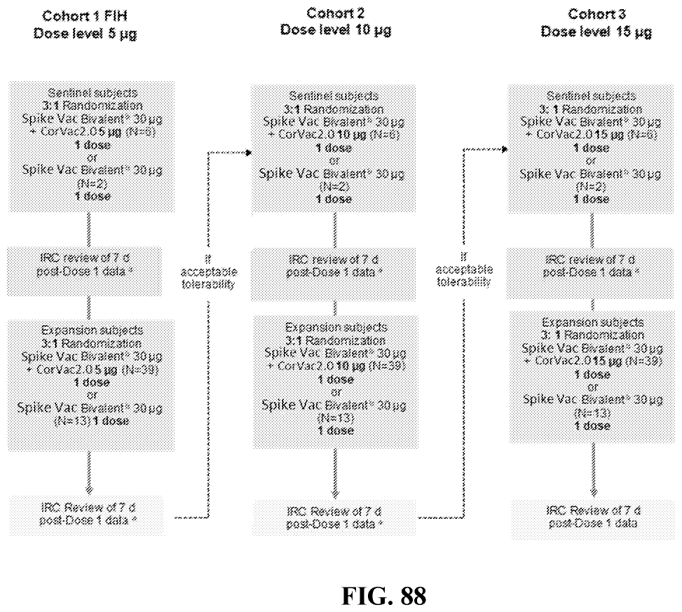

The experimental protocol used in the present example to determine B and T cell clonotypes is depicted in FIG. 87, and is similar to that described in Andersen, Rikke Sick, et al. “Parallel detection of antigen-specific T cell responses by combinatorial encoding of MHC multimers.” Nature protocols 7.5 (2012): 891-902, the contents of which is incorporated by reference herein in their entirety. In brief, Spike-specific T cells were labeled by staining splenocytes with fluorescently-labelled Spike pMHC tetramers (fluorescently labeled multimeric complexes of peptide-MHC (molecular entities recognized by T cell receptosr)) at 37° C. for 15 minutes and spike-specific B cells were labeled by staining splenocytes with fluorescently-labelled, tetramerized Spike bait protein. Cells were washed and labelled with CITE Abs, and surface stained for CD3, CD19, and CD11b for sorting. Sorted cells were collected from each animal, and animals from each group were pooled for single cell library generation.

Clonotype networks: Sequence-based distance matrices for VJ and VDJ sequences were calculated using alignment distance (BLOSUM62 matrix) of CDR3 aa sequences.

Clustering cells by similarity of CDR3-sequences: Each fully connected subnetwork shown in FIG. 87 represents a “clonotype cluster” and each dot represents cells with identical receptor configurations (i.e., TCR sequence), with the size of the dot corresponding to the number of cells detecting having a given receptor configuration.

Clonal expansion: the bar graph in FIG. 87 shows the number of cells belonging to an expanded clonotype per category in stacked bar plot. As shown in the bag graph, introduction of CorVac2.0 trends towards higher conal explansion, with clonality of Spike-specific T cells not negatively impacted.

TABLE 9
Group 1 Construct-maps

| CONSTRUCT | Segment | | | | |
|---|---|---|---|---|---|
| Name | NAME | AA SEQUENCE | DNA SEQUENCE | START | END |
| RS C1 GSS | SP1 | MRVMAPRTLILLLSGALALTETWA | ATGAGAGTGATGGCCCCCAGAACCCTGATCCTGCTGCTGTCT | 1 | 78 |
| linkers | | GS | GGCGCCCTGGCCCTGACAGAGACATGGGCCGGAAGC | | |
| RS C1 GSS | start GSS | GGSGGGGSGG | GGAGGTTCCGGAGGAGGCGGCAGTGGCGGC | 79 | 108 |
| linkers | | | | | |
| RS C1 GSS | nucleocapsid | SDNGPQNQRNAPRITFGGPSDSTGS | TCTGATAATGGACCCCAAAATCAGCGAAATGCACCCCGCATT | 109 | 1362 |
| linkers | phosphoprotein | NONGERSGARSKQRRPQGLPNNTA | ACGTTTGGTGGACCCTCAGATTCAACTGGCAGTAACCAGAAT | | |
| | | SWFTALTQHGKEDLKFPRGQGVPI | GGAGAACGCAGTGGGGCGCGATCAAAACAACGTCGGCCCCA | | |
| | | NTNSSPDDQIGYYRRATRRIRGGDG | AGGTTTACCCAATAATACTGCGTCTTGGTTCACCGCTCTCACT | | |
| | | KMKDLSPRWYFYYLGTGPEAGLPY | CAACATGGCAAGGAAGACCTTAAATTCCCTCGAGGACAAGGC | | |
| | | GANKDGIIWVATEGALNTPKDHIGT | GTTCCAATTAACACCAATAGCAGTCCAGATGACCAAATTGGC | | |
| | | RNPANNAAIVLQLPQGTTLPKGFYA | TACTACCGAAGAGCTACCAGACGAATTCGTGGTGGTGACGGT | | |
| | | EGSRGGSQASSRSSSRSRNSSRNSTP | AAAATGAAAGATCTCAGTCCAAGATGGTATTTCTACTACCTA | | |
| | | GSSRGTSPARMAGNGGDAALALLL | GGAACTGGGCCAGAAGCTGGACTTCCCTATGGTGCTAACAAA | | |
| | | LDRLNQLESKMSGKGQQQQGQTV | GACGGCATCATATGGGTTGCAACTGAGGGAGCCTTGAATACA | | |
| | | TKKSAAEASKKPRQKRTATKAYNV | CCAAAAGATCACATTGGCACCCGCAATCCTGCTAACAATGCT | | |
| | | TQAFGRRGPEQTQGNFGDQELIRQ | GCAATCGTGCTACAACTTCCTCAAGGAACAACATTGCCAAAA | | |
| | | GTDYKHWPQIAQFAPSASAFFGMS | GGCTTCTACGCAGAAGGGAGCAGAGGCGGCAGTCAAGCCTCT | | |
| | | RIGMEVTPSGTWLTYTGAIKLDDK | TCTCGTTCCTCATCACGTAGTCGCAACAGTTCAAGAAATTCAA | | |
| | | DPNFKDQVILLNKHIDAYKTFPPTE | CTCCAGGCAGCAGTAGGGGAACTTCTCCTGCTAGAATGGCTG | | |
| | | PKKDKKKKADETQALPQRQKKQQ | GCAATGGCGGTGATGCTGCTCTTGCTTTGCTGCTGCTTGACAG | | |
| | | TVTLLPAADLDDFSKQLQQSMSSA | ATTGAACCAGCTTGAGAGCAAAATGTCTGGTAAAGGCCAACA | | |
| | | DSTQA | ACAACAAGGCCAAACTGTCACTAAGAAATCTGCTGCTGAGGC | | |
| | | | TTCTAAGAAGCCTCGGCAAAAACGTACTGCCACTAAAGCATA | | |
| | | | CAATGTAACACAAGCTTTCGGCAGACGTGGTCCAGAACAAAC | | |
| | | | CCAAGGAAATTTTGGGGACCAGGAACTAATCAGACAAGGAA | | |
| | | | CTGATTACAAACATTGGCCGCAAATTGCACAATTTGCCCCCA | | |
| | | | GCGCTTCAGCGTTCTTCGGAATGTCGCGCATTGGCATGGAAG | | |
| | | | TCACACCTTCGGGAACGTGGTTGACCTACACAGGTGCCATCA | | |
| | | | AATTGGATGACAAAGATCCAAATTTCAAAGATCAAGTCATTT | | |
| | | | TGCTGAATAAGCATATTGACGCATACAAAACATTCCCACCAA | | |
| | | | CAGAGCCTAAAAAGGACAAAAAGAAGAAGGCTGATGAAACT | | |
| | | | CAAGCCTTACCGCAGAGACAGAAGAAACAGCAAACTGTGAC | | |
| | | | TCTTCTTCCTGCTGCAGATTTGGATGATTTCTCCAAACAATTG | | |
| | | | CAACAATCCATGAGCAGTGCTGACTCAACTCAGGCC | | |
| RS C1 GSS | middle | GGSGGGGSGG | GGAGGTTCTGGGGGCGGCGGTTCAGGGGGC | 1363 | 1392 |
| linkers | GSS | | | | |
| RS C1 GSS | ORF9b | DPKISEMHPALRLVDPQIQLAVTRM | GACCCCAAAATCAGCGAAATGCACCCCGCATTACGTTTGGTG | 1393 | 1680 |
| linkers | protein | ENAVGRDQNNVGPKVYPIILRLGSP | GACCCTCAGATTCAACTGGCAGTAACCAGAATGGAGAACGCA | | |
| | | LSLNMARKTLNSLEDKAFQLTPIAV | GTGGGGCGCGATCAAAACAACGTCGGCCCCAAGGTTTACCCA | | |
| | | QMTKLATTEELPDEFVVVTVK | ATAATACTGCGTCTTGGTTCACCGCTCTCACTCAACATGGCAA | | |
| | | | GGAAGACCTTAAATTCCCTCGAGGACAAGGCGTTCCAATTAA | | |
| | | | CACCAATAGCAGTCCAGATGACCAAATTGGCTACTACCGAAG | | |
| | | | AGCTACCAGACGAATTCGTGGTGGTGACGGTAAAA | | |
| RS C1 GSS | middle | GGSGGGGSGG | GGAGGTTCTGGGGGCGGCGGTTCAGGGGGC | 1681 | 1710 |
| linkers | GSS | | | | |
| RS C1 GSS | cleavage | YHFF | TACCATTTCTTT | 1711 | 1722 |
| linkers | linker | | | | |
| RS C1 GSS | ORF1ab | HTTDPSFLGRYMSAL | CACACAACTGATCCTAGTTTTCTGGGTAGGTACATGTCAGCAT | 1723 | 1767 |
| linkers | NSP3 | | TA | | |
| | 1636- | | | | |
| | 1650 | | | | |
| RS C1 GSS | ORF1ab | FADDLNQLTGY | TTTGCTGATGATTTAAACCAGTTAACTGGTTAT | 1768 | 1800 |
| linkers | NSP3 | | | | |
| | 1937- | | | | |
| | 1947 | | | | |
| RS C1 GSS | ORF1ab | HTDFSSEIIGY | CATACTGACTTTTCAAGTGAAATCATAGGATAC | 1801 | 1833 |
| linkers | NSP4 | | | | |
| | 2799- | | | | |
| | 2809 | | | | |
| RS C1 GSS | ORF1ab | QLMCQPILL | CAGCTTATGTGTCAACCTATACTGTTA | 1834 | 1860 |
| linkers | NSP3 | | | | |
| | 2563- | | | | |
| | 2571 | | | | |
| RS C1 GSS | ORF1ab | | | 1861 | 1893 |
| linkers | NSP3 | AEAELAKNVSL | GCAGAAGCTGAACTTGCAAAGAATGTGTCCTTA | | |
| | 2616- | | | | |
| | 2626 | | | | |
| RS C1 GSS | ORF1ab | ILGTVSWNL | ATTCTTGGAACTGTTTCTTGGAATTTG | 1894 | 1920 |
| linkers | NSP3 | | | | |
| | 1367- | | | | |
| | 1375 | | | | |
| RS C1 GSS | cleavage | KRRY | AAGCGTCGCTAT | 1921 | 1932 |
| linkers | linker | | | | |
| RS C1 GSS | ORF1ab | LLSAGIFGA | TTATTATCAGCTGGTATTTTTGGTGCT | 1933 | 1959 |
| linkers | NSP3 | | | | |
| | 1148- | | | | |
| | 1156 | | | | |
| RS C1 GSS | ORF1ab | ITDVFYKENSY | ATTACGGATGTTTTCTACAAAGAAAACAGTTAC | 1960 | 1992 |
| linkers | NSP3 | | | | |
| | 1863- | | | | |
| | 1873 | | | | |
| RS C1 GSS | ORF1ab | KVPTDNYITTY | AAAGTGCCAACAGACAATTATATAACCACTTAC | 1993 | 2025 |
| linkers | NSP3 | | | | |
| | 1319- | | | | |
| | 1329 | | | | |
| RS C1 GSS | cleavage | ARMA | GCGCGGATGGCT | 2026 | 2037 |
| linkers | linker | | | | |
| RS C1 GSS | ORF1ab | APKEIIFLEGETL | GCCCCAAAAGAAATTATCTTCTTAGAGGGAGAAACACTT | 2038 | 2076 |
| linkers | NSP2 | | | | |
| | 735-747 | | | | |
| RS C1 GSS | ORF1ab | FGDDTVIEV | TTTGGTGATGACACTGTGATAGAAGTG | 2077 | 2103 |
| linkers | INSP3 | | | | |
| | 825-833 | | | | |
| RS C1 GSS | ORF1ab | AIILASFSAST | GCCATTATTTTGGCATCTTTTTCTGCTTCCACA | 2104 | 2136 |
| linkers | NSP2 | | | | |
| | 474-484 | | | | |
| RS C1 GSS | cleavage | RRMA | CGCCGTATGGCA | 2137 | 2148 |
| linkers | linker | | | | |
| RS C1 GSS | ORF1ab | MVTNNTFTLK | ATGGTAACAAACAATACCTTCACACTCAAA | 2149 | 2178 |
| linkers | NSP2 | | | | |
| | 807-816 | | | | |
| RS C1 GSS | ORF1ab | VPHVGEIPVAYRKVLL | GTCCCTCATGTGGGCGAAATACCAGTGGCTTACCGCAAGGTT | 2179 | 2226 |
| linkers | NSP1 | | CTTCTT | | |
| | 108-123 | | | | |
| RS C1 GSS | ORF1ab | KTIQPRVEK | AAGACTATTCAACCAAGGGTTGAAAAG | 2227 | 2253 |
| linkers | NSP2 | | | | |
| | 282-290 | | | | |
| RS C1 GSS | NSP3 | YLFDESGEFKL | TACTTATTTGATGAGTCTGGTGAGTTTAAATTG | 2254 | 2286 |
| linkers | ORF1ab | | | | |
| | 906-916 | | | | |
| RS C1 GSS | ORF1ab | SEVGPEHSLAEY | TCAGAAGTAGGACCTGAGCATAGTCTTGCCGAATAC | 2287 | 2322 |
| linkers | NSP2 | | | | |
| | 376-387 | | | | |
| RS C1 GSS | ORF1ab | YIFFASFYY | TACATCTTCTTTGCATCATTTTATTAT | 2323 | 2349 |
| linkers | NSP3 | | | | |
| | 2384- | | | | |
| | 2392 | | | | |
| RS C1 GSS | cleavage | KRNG | AAACGTAACGGG | 2350 | 2361 |
| linkers | linker | | | | |
| RS C1 GSS | middle | GGSGGGGSGG | GGAGGTTCTGGGGGCGGCGGTTCAGGGGGC | 2362 | 2391 |
| linkers | GSS | | | | |
| RS C1 GSS | ORF3a | DLFMRIFTIGTVTLKQGEIKDATPSD | AGCAAGGTGAAATCAAGGATGCTACTCCTTCAGATTTTGTTC | 2392 | 3213 |
| linkers | protein | FVRATATIPIQASLPFGWLIVGVALL | GATTTGTTTATGAGAATCTTCACAATTGGAACTGTAACTTTGA | | |
| | | AVFQSASKIITLKKRWQLALSKGVH | GCGCTACTGCAACGATACCGATACAAGCCTCACTCCCTTTCG | | |
| | | FVCNLLLLFVTVYSHLLLVAAGLEA | GATGGCTTATTGTTGGCGTTGCACTTCTTGCTGTTTTTCAGAG | | |
| | | PFLYLYALVYFLQSINFVRIIMRLWL | CGCTTCCAAAATCATAACCCTCAAAAAGAGATGGCAACTAGC | | |
| | | CWKCRSKNPLLYDANYFLCWHTN | ACTCTCCAAGGGTGTTCACTTTGTTTGCAACTTGCTGTTGTTG | | |
| | | CYDYCIPYNSVTSSIVITSGDGTTSPI | TTTGTAACAGTTTACTCACACCTTTTGCTCGTTGCTGCTGGCCT | | |
| | | SEHDYQIGGYTEKWESGVKDCVVL | TGAAGCCCCTTTTCTCTATCTTTATGCTTTAGTCTACTTCTTGC | | |
| | | HSYFTSDYYQLYSTQLSTDTGVEH | AGAGTATAAACTTTGTAAGAATAATAATGAGGCTTTGGCTTT | | |
| | | VTFFIYNKIVDEPEEHVQIHTIDGSS | GCTGGAAATGCCGTTCCAAAAACCCATTACTTTATGATGCCA | | |
| | | GVVNPVMEPIYDEPTTTTSVPL | ACTATTTTCTTTGCTGGCATACTAATTGTTACGACTATTGTAT | | |
| | | | ACCTTACAATAGTGTAACTTCTTCAATTGTCATTACTTCAGGT | | |
| | | | GATGGCACAACAAGTCCTATTTCTGAACATGACTACCAGATT | | |
| | | | GGTGGTTATACTGAAAAATGGGAATCTGGAGTAAAAGACTGT | | |
| | | | GTTGTATTACACAGTTACTTCACTTCAGACTATTACCAGCTGT | | |
| | | | ACTCAACTCAATTGAGTACAGACACTGGTGTTGAACATGTTA | | |
| | | | CCTTCTTCATCTACAATAAAATTGTTGATGAGCCTGAAGAACA | | |
| | | | TGTCCAAATTCACACAATCGACGGTTCATCCGGAGTTGTTAAT | | |
| | | | CCAGTAATGGAACCAATTTATGATGAACCGACGACGACTACT | | |
| | | | AGCGTGCCTTTG | | |
| RS C1 GSS | middle | GGSGGGGSGG | GGAGGTTCTGGGGGCGGCGGTTCAGGGGGC | 3214 | 3243 |
| linkers | GSS | | | | |
| RS C1 GSS | membrane | ADSNGTITVEELKKLLEQWNLVIGF | GCAGATTCCAACGGTACTATTACCGTTGAAGAGCTTAAAAAG | 3244 | 3906 |
| linkers | glycoprotein | LFLTWICLLQFAYANRNRFLYIIKLI | CTCCTTGAACAATGGAACCTAGTAATAGGTTTCCTATTCCTTA | | |
| | | FLWLLWPVTLACFVLAAVYRINWI | CATGGATTTGTCTTCTACAATTTGCCTATGCCAACAGGAATAG | | |
| | | TGGIAIAMACLVGLMWLSYFIASFR | GTTTTTGTATATAATTAAGTTAATTTTCCTCTGGCTGTTATGGC | | |
| | | LFARTRSMWSFNPETNILLNVPLHG | CAGTAACTTTAGCTTGTTTTGTGCTTGCTGCTGTTTACAGAAT | | |
| | | TILTRPLLESELVIGAVILRGHLRIAG | AAATTGGATCACCGGTGGAATTGCTATCGCAATGGCTTGTCTT | | |
| | | HHLGRCDIKDLPKEITVATSRTLSY | GTAGGCTTGATGTGGCTCAGCTACTTCATTGCTTCTTTCAGAC | | |
| | | YKLGASQRVAGDSGFAAYSRYRIG | TGTTTGCGCGTACGCGTTCCATGTGGTCATTCAATCCAGAAAC | | |
| | | NYKLNTDHSSSSDNIALLVQ | TAACATTCTTCTCAACGTGCCACTCCATGGCACTATTCTGACC | | |
| | | | AGACCGCTTCTAGAAAGTGAACTCGTAATCGGAGCTGTGATC | | |
| | | | CTTCGTGGACATCTTCGTATTGCTGGACACCATCTAGGACGCT | | |
| | | | GTGACATCAAGGACCTGCCTAAAGAAATCACTGTTGCTACAT | | |
| | | | CACGAACGCTTTCTTATTACAAATTGGGAGCTTCGCAGCGTGT | | |
| | | | AGCAGGTGACTCAGGTTTTGCTGCATACAGTCGCTACAGGAT | | |
| | | | TGGCAACTATAAATTAAACACAGACCATTCCAGTAGCAGTGA | | |
| | | | CAATATTGCTTTGCTTGTACAG | | |
| RS C1 GSS | end GSS | GGSLGGGGSG | GGTGGTTCTCTTGGCGGAGGGGGTTCGGGA | 3907 | 3936 |
| linkers | | | | | |
| RS C1 GSS | MITD | IVGIVAGLAVLAVVVIGAVVATVM | ATCGTGGGAATTGTGGCAGGACTGGCAGTGCTGGCCGTGGTG | 3937 | 4107 |
| linkers | | CRRKSSGGKGGSYSQAASSDSAQG | GTGATCGGAGCCGTGGTGGCTACCGTGATGTGCAGACGGAAG | | |
| | | SDVSLTA** | TCCAGCGGAGGCAAGGGCGGCAGCTACAGCCAGGCCGCCAG | | |
| | | | CTCTGATAGCGCCCAGGGCAGCGACGTGTCACTGACAGCCTA | | |
| | | | GTAA | | |
| RS C2 GSS | ISP1 | MRVMAPRTLILLLSGALALTETWA | ATGAGAGTGATGGCCCCCAGAACCCTGATCCTGCTGCTGTCT | 1 | 78 |
| linkers- | | GS | GGCGCCCTGGCCCTGACAGAGACATGGGCCGGAAGC | | |
| inverted | | | | | |
| RS C2 GSS | start GSS | GGSGGGGSGG | GGAGGTTCCGGAGGAGGCGGCAGTGGCGGC | 79 | 108 |
| linkers- | | | | | |
| inverted | | | | | |
| RS C2 GSS | membrane | ADSNGTITVEELKKLLEQWNLVIGF | GCAGATTCCAACGGTACTATTACCGTTGAAGAGCTTAAAAAG | 109 | 771 |
| linkers- | glycoprotein | LFLTWICLLQFAYANRNRFLYIIKLI | CTCCTTGAACAATGGAACCTAGTAATAGGTTTCCTATTCCTTA | | |
| inverted | | FLWLLWPVTLACFVLAAVYRINWI | CATGGATTTGTCTTCTACAATTTGCCTATGCCAACAGGAATAG | | |
| | | TGGIAIAMACLVGLMWLSYFIASFR | GTTTTTGTATATAATTAAGTTAATTTTCCTCTGGCTGTTATGGC | | |
| | | LFARTRSMWSFNPETNILLNVPLHG | CAGTAACTTTAGCTTGTTTTGTGCTTGCTGCTGTTTACAGAAT | | |
| | | TILTRPLLESELVIGAVILRGHLRIAG | AAATTGGATCACCGGTGGAATTGCTATCGCAATGGCTTGTCTT | | |
| | | HHLGRCDIKDLPKEITVATSRTLSY | GTAGGCTTGATGTGGCTCAGCTACTTCATTGCTTCTTTCAGAC | | |
| | | YKLGASQRVAGDSGFAAYSRYRIG | TGTTTGCGCGTACGCGTTCCATGTGGTCATTCAATCCAGAAAC | | |
| | | NYKLNTDHSSSSDNIALLVQ | TAACATTCTTCTCAACGTGCCACTCCATGGCACTATTCTGACC | | |
| | | | AGACCGCTTCTAGAAAGTGAACTCGTAATCGGAGCTGTGATC | | |
| | | | CTTCGTGGACATCTTCGTATTGCTGGACACCATCTAGGACGCT | | |
| | | | GTGACATCAAGGACCTGCCTAAAGAAATCACTGTTGCTACAT | | |
| | | | CACGAACGCTTTCTTATTACAAATTGGGAGCTTCGCAGCGTGT | | |
| | | | AGCAGGTGACTCAGGTTTTGCTGCATACAGTCGCTACAGGAT | | |
| | | | TGGCAACTATAAATTAAACACAGACCATTCCAGTAGCAGTGA | | |
| | | | CAATATTGCTTTGCTTGTACAG | | |
| RS C2 GSS | middle | GGSGGGGSGG | GGAGGTTCTGGGGGCGGCGGTTCAGGGGGC | 772 | 801 |
| linkers- | GSS | | | | |
| inverted | | | | | |
| RS C2 GSS | ORF3a | DLFMRIFTIGTVTLKQGEIKDATPSD | GATTTGTTTATGAGAATCTTCACAATTGGAACTGTAACTTTGA | 802 | 1623 |
| linkers- | protein | FVRATATIPIQASLPFGWLIVGVALL | AGCAAGGTGAAATCAAGGATGCTACTCCTTCAGATTTTGTTC | | |
| inverted | | AVFQSASKIITLKKRWQLALSKGVH | GCGCTACTGCAACGATACCGATACAAGCCTCACTCCCTTTCG | | |
| | | FVCNLLLLFVTVYSHLLLVAAGLEA | GATGGCTTATTGTTGGCGTTGCACTTCTTGCTGTTTTTCAGAG | | |
| | | PFLYLYALVYFLQSINFVRIIMRLWL | CGCTTCCAAAATCATAACCCTCAAAAAGAGATGGCAACTAGC | | |
| | | CWKCRSKNPLLYDANYFLCWHTN | ACTCTCCAAGGGTGTTCACTTTGTTTGCAACTTGCTGTTGTTG | | |
| | | CYDYCIPYNSVTSSIVITSGDGTTSPI | TTTGTAACAGTTTACTCACACCTTTTGCTCGTTGCTGCTGGCCT | | |
| | | SEHDYQIGGYTEKWESGVKDCVVL | TGAAGCCCCTTTTCTCTATCTTTATGCTTTAGTCTACTTCTTGC | | |
| | | HSYFTSDYYQLYSTQLSTDTGVEH | AGAGTATAAACTTTGTAAGAATAATAATGAGGCTTTGGCTTT | | |
| | | VTFFIYNKIVDEPEEHVQIHTIDGSS | GCTGGAAATGCCGTTCCAAAAACCCATTACTTTATGATGCCA | | |
| | | GVVNPVMEPIYDEPTTTTSVPL | ACTATTTTCTTTGCTGGCATACTAATTGTTACGACTATTGTAT | | |
| | | | ACCTTACAATAGTGTAACTTCTTCAATTGTCATTACTTCAGGT | | |
| | | | GATGGCACAACAAGTCCTATTTCTGAACATGACTACCAGATT | | |
| | | | GGTGGTTATACTGAAAAATGGGAATCTGGAGTAAAAGACTGT | | |
| | | | GTTGTATTACACAGTTACTTCACTTCAGACTATTACCAGCTGT | | |
| | | | ACTCAACTCAATTGAGTACAGACACTGGTGTTGAACATGTTA | | |
| | | | CCTTCTTCATCTACAATAAAATTGTTGATGAGCCTGAAGAACA | | |
| | | | TGTCCAAATTCACACAATCGACGGTTCATCCGGAGTTGTTAAT | | |
| | | | CCAGTAATGGAACCAATTTATGATGAACCGACGACGACTACT | | |
| | | | AGCGTGCCTTTG | | |
| RS C2 GSS | middle | GGSGGGGSGG | GGAGGTTCTGGGGGCGGCGGTTCAGGGGGC | 1624 | 1653 |
| linkers- | GSS | | | | |
| inverted | | | | | |
| RS C2 GSS | cleavage | YHFF | TACCATTTCTTT | 1654 | 1665 |
| linkers- | linker | | | | |
| inverted | | | | | |
| RS C2 GSS | ORF1ab | HTTDPSFLGRYMSAL | CACACAACTGATCCTAGTTTTCTGGGTAGGTACATGTCAGCAT | 1666 | 1710 |
| linkers- | NSP3 | | | | |
| inverted | 1636- | | TA | | |
| | 1650 | | | | |
| RS C2 GSS | ORF1ab | FADDLNQLTGY | TTTGCTGATGATTTAAACCAGTTAACTGGTTAT | 1711 | 1743 |
| linkers- | NSP3 | | | | |
| inverted | 1937- | | | | |
| | 1947 | | | | |
| RS C2 GSS | ORF1ab | HTDFSSEIIGY | CATACTGACTTTTCAAGTGAAATCATAGGATAC | 1744 | 1776 |
| linkers- | NSP4 | | | | |
| inverted | 2799- | | | | |
| | 2809 | | | | |
| RS C2 GSS | ORF1ab | QLMCQPILL | CAGCTTATGTGTCAACCTATACTGTTA | 1777 | 1803 |
| linkers- | NSP3 | | | | |
| inverted | 2563- | | | | |
| | 2571 | | | | |
| RS C2 GSS | ORF1ab | AEAELAKNVSL | GCAGAAGCTGAACTTGCAAAGAATGTGTCCTTA | 1804 | 1836 |
| linkers- | NSP3 | | | | |
| inverted | 2616- | | | | |
| | 2626 | | | | |
| RS C2 GSS | ORF1ab | ILGTVSWNL | ATTCTTGGAACTGTTTCTTGGAATTTG | 1837 | 1863 |
| linkers- | NSP3 | | | | |
| inverted | 1367- | | | | |
| | 1375 | | | | |
| RS C2 GSS | cleavage | KRRY | AAGCGTCGCTAT | 1864 | 1875 |
| linkers- | linker | | | | |
| inverted | | | | | |
| RS C2 GSS | ORF1ab | LLSAGIFGA | TTATTATCAGCTGGTATTTTTGGTGCT | 1876 | 1902 |
| linkers- | NSP3 | | | | |
| inverted | 1148- | | | | |
| | 1156 | | | | |
| RS C2 GSS | ORF1ab | ITDVFYKENSY | ATTACGGATGTTTTCTACAAAGAAAACAGTTAC | 1903 | 1935 |
| linkers- | NSP3 | | | | |
| inverted | 1863- | | | | |
| | 1873 | | | | |
| RS C2 GSS | ORF1ab | KVPTDNYITTY | AAAGTGCCAACAGACAATTATATAACCACTTAC | 1936 | 1968 |
| linkers- | NSP3 | | | | |
| inverted | 1319- | | | | |
| | 1329 | | | | |
| RS C2 GSS | cleavage | ARMA | GCGCGGATGGCT | 1969 | 1980 |
| linkers- | linker | | | | |
| inverted | | | | | |
| RS C2 GSS | ORF1ab | APKEIIFLEGETL | GCCCCAAAAGAAATTATCTTCTTAGAGGGAGAAACACTT | 1981 | 2019 |
| linkers- | NSP2 | | | | |
| inverted | 735-747 | | | | |
| RS C2 GSS | ORF1ab | FGDDTVIEV | TTTGGTGATGACACTGTGATAGAAGTG | 2020 | 2046 |
| linkers- | NSP3 | | | | |
| inverted | 825-833 | | | | |
| RS C2 GSS | ORF1ab | AIILASFSAST | GCCATTATTTTGGCATCTTTTTCTGCTTCCACA | 2047 | 2079 |
| linkers- | NSP2 | | | | |
| inverted | 474-484 | | | | |
| RS C2 GSS | cleavage | RRMA | CGCCGTATGGCA | 2080 | 2091 |
| linkers- | linker | | | | |
| inverted | | | | | |
| RS C2 GSS | ORF1ab | MVTNNTFTLK | ATGGTAACAAACAATACCTTCACACTCAAA | 2092 | 2121 |
| linkers- | NSP2 | | | | |
| inverted | 807-816 | | | | |
| RS C2 GSS | ORF1ab | VPHVGEIPVAYRKVLL | GTCCCTCATGTGGGCGAAATACCAGTGGCTTACCGCAAGGTT | 2122 | 2169 |
| linkers- | NSP1 | | CTTCTT | | |
| inverted | 108-123 | | | | |
| RS C2 GSS | ORF1ab | KTIQPRVEK | AAGACTATTCAACCAAGGGTTGAAAAG | 2170 | 2196 |
| linkers- | NSP2 | | | | |
| inverted | 282-290 | | | | |
| RS C2 GSS | ORF1ab | YLFDESGEFKL | TACTTATTTGATGAGTCTGGTGAGTTTAAATTG | 2197 | 2229 |
| linkers- | NSP3 | | | | |
| inverted | 906-916 | | | | |
| RS C2 GSS | ORF1ab | SEVGPEHSLAEY | TCAGAAGTAGGACCTGAGCATAGTCTTGCCGAATAC | 12230 | 2265 |
| linkers- | NSP2 | | | | |
| inverted | 376-387 | | | | |
| RS C2 GSS | ORF1ab | YIFFASFYY | TACATCTTCTTTGCATCATTTTATTAT | 2266 | 2292 |
| linkers- | NSP3 | | | | |
| inverted | 12384- | | | | |
| | 2392 | | | | |
| RS C2 GSS | cleavage | KRNG | AAACGTAACGGG | 2293 | 2304 |
| linkers- | linker | | | | |
| inverted | | | | | |
| RS C2 GSS | middle | GGSGGGGSGG | GGAGGTTCTGGGGGCGGCGGTTCAGGGGGC | 2305 | 2334 |
| linkers- | GSS | | | | |
| inverted | | | | | |
| RS C2 GSS | ORF9b | DPKISEMHPALRLVDPQIQLAVTRM | GACCCCAAAATCAGCGAAATGCACCCCGCATTACGTTTGGTG | 2335 | 2622 |
| linkers- | protein | ENAVGRDQNNVGPKVYPIILRLGSP | GACCCTCAGATTCAACTGGCAGTAACCAGAATGGAGAACGCA | | |
| inverted | | LSLNMARKTLNSLEDKAFQLTPIAV | GTGGGGCGCGATCAAAACAACGTCGGCCCCAAGGTTTACCCA | | |
| | | QMTKLATTEELPDEFVVVTVK | ATAATACTGCGTCTTGGTTCACCGCTCTCACTCAACATGGCAA | | |
| | | | GGAAGACCTTAAATTCCCTCGAGGACAAGGCGTTCCAATTAA | | |
| | | | CACCAATAGCAGTCCAGATGACCAAATTGGCTACTACCGAAG | | |
| | | | AGCTACCAGACGAATTCGTGGTGGTGACGGTAAAA | | |
| RS C2 GSS | middle | GGSGGGGSGG | GGAGGTTCTGGGGGCGGCGGTTCAGGGGGC | 2623 | 2652 |
| linkers- | GSS | | | | |
| inverted | | | | | |
| RS C2 GSS | nucleocapsid | SDNGPQNQRNAPRITFGGPSDSTGS | TCTGATAATGGACCCCAAAATCAGCGAAATGCACCCCGCATT | 2653 | 3906 |
| linkers- | phosphoprotein | NQNGERSGARSKQRRPQGLPNNTA | ACGTTTGGTGGACCCTCAGATTCAACTGGCAGTAACCAGAAT | | |
| inverted | | SWFTALTQHGKEDLKFPRGQGVPI | GGAGAACGCAGTGGGGCGCGATCAAAACAACGTCGGCCCCA | | |
| | | NTNSSPDDQIGYYRRATRRIRGGDG | AGGTTTACCCAATAATACTGCGTCTTGGTTCACCGCTCTCACT | | |
| | | KMKDLSPRWYFYYLGTGPEAGLPY | CAACATGGCAAGGAAGACCTTAAATTCCCTCGAGGACAAGGC | | |
| | | GANKDGIIWVATEGALNTPKDHIGT | GTTCCAATTAACACCAATAGCAGTCCAGATGACCAAATTGGC | | |
| | | RNPANNAAIVLQLPQGTTLPKGFYA | TACTACCGAAGAGCTACCAGACGAATTCGTGGTGGTGACGGT | | |
| | | EGSRGGSQASSRSSSRSRNSSRNSTP | AAAATGAAAGATCTCAGTCCAAGATGGTATTTCTACTACCTA | | |
| | | GSSRGTSPARMAGNGGDAALALLL | GGAACTGGGCCAGAAGCTGGACTTCCCTATGGTGCTAACAAA | | |
| | | LDRLNQLESKMSGKGQQQQGQTV | GACGGCATCATATGGGTTGCAACTGAGGGAGCCTTGAATACA | | |
| | | TKKSAAEASKKPRQKRTATKAYNV | CCAAAAGATCACATTGGCACCCGCAATCCTGCTAACAATGCT | | |
| | | TQAFGRRGPEQTQGNFGDQELIRQ | GCAATCGTGCTACAACTTCCTCAAGGAACAACATTGCCAAAA | | |
| | | GTDYKHWPQIAQFAPSASAFFGMS | GGCTTCTACGCAGAAGGGAGCAGAGGCGGCAGTCAAGCCTCT | | |
| | | RIGMEVTPSGTWLTYTGAIKLDDK | TCTCGTTCCTCATCACGTAGTCGCAACAGTTCAAGAAATTCAA | | |
| | | DPNFKDQVILLNKHIDAYKTFPPTE | CTCCAGGCAGCAGTAGGGGAACTTCTCCTGCTAGAATGGCTG | | |
| | | PKKDKKKKADETQALPQRQKKQQ | GCAATGGCGGTGATGCTGCTCTTGCTTTGCTGCTGCTTGACAG | | |
| | | TVTLLPAADLDDFSKQLQQSMSSA | ATTGAACCAGCTTGAGAGCAAAATGTCTGGTAAAGGCCAACA | | |
| | | DSTQA | ACAACAAGGCCAAACTGTCACTAAGAAATCTGCTGCTGAGGC | | |
| | | | TTCTAAGAAGCCTCGGCAAAAACGTACTGCCACTAAAGCATA | | |
| | | | CAATGTAACACAAGCTTTCGGCAGACGTGGTCCAGAACAAAC | | |
| | | | CCAAGGAAATTTTGGGGACCAGGAACTAATCAGACAAGGAA | | |
| | | | CTGATTACAAACATTGGCCGCAAATTGCACAATTTGCCCCCA | | |
| | | | GCGCTTCAGCGTTCTTCGGAATGTCGCGCATTGGCATGGAAG | | |
| | | | TCACACCTTCGGGAACGTGGTTGACCTACACAGGTGCCATCA | | |
| | | | AATTGGATGACAAAGATCCAAATTTCAAAGATCAAGTCATTT | | |
| | | | TGCTGAATAAGCATATTGACGCATACAAAACATTCCCACCAA | | |
| | | | CAGAGCCTAAAAAGGACAAAAAGAAGAAGGCTGATGAAACT | | |
| | | | CAAGCCTTACCGCAGAGACAGAAGAAACAGCAAACTGTGAC | | |
| | | | TCTTCTTCCTGCTGCAGATTTGGATGATTTCTCCAAACAATTG | | |
| | | | CAACAATCCATGAGCAGTGCTGACTCAACTCAGGCC | | |
| RS C2 GSS | end GSS | GGSLGGGGSG | GGTGGTTCTCTTGGCGGAGGGGGTTCGGGA | 3907 | 3936 |
| linkers- | | | | | |
| inverted | | | | | |
| RS C2 GSS | MITD | IVGIVAGLAVLAVVVIGAVVATVM | ATCGTGGGAATTGTGGCAGGACTGGCAGTGCTGGCCGTGGTG | 3937 | 4107 |
| linkers- | | CRRKSSGGKGGSYSQAASSDSAQG | GTGATCGGAGCCGTGGTGGCTACCGTGATGTGCAGACGGAAG | | |
| inverted | | SDVSLTA** | TCCAGCGGAGGCAAGGGCGGCAGCTACAGCCAGGCCGCCAG | | |
| | | | CTCTGATAGCGCCCAGGGCAGCGACGTGTCACTGACAGCCTA | | |
| | | | GTAA | | |
| RS C3 2A | SP1 | MRVMAPRTLILLLSGALALTETWA | ATGAGAGTGATGGCCCCCAGAACCCTGATCCTGCTGCTGTCT | 1 | 78 |
| linkers | | GS | GGCGCCCTGGCCCTGACAGAGACATGGGCCGGAAGC | | |
| RS C3 2A | start GSS | GGSGGGGSGG | GGAGGTTCCGGAGGAGGCGGCAGTGGCGGC | 79 | 108 |
| linkers | | | | | |
| RS C3 2A | nucleocapsid | SDNGPQNQRNAPRITFGGPSDSTGS | TCTGATAATGGACCCCAAAATCAGCGAAATGCACCCCGCATT | 109 | 1362 |
| linkers | phosphoprotein | NQNGERSGARSKQRRPQGLPNNTA | ACGTTTGGTGGACCCTCAGATTCAACTGGCAGTAACCAGAAT | | |
| | | SWFTALTQHGKEDLKFPRGQGVPI | GGAGAACGCAGTGGGGCGCGATCAAAACAACGTCGGCCCCA | | |
| | | NTNSSPDDQIGYYRRATRRIRGGDG | AGGTTTACCCAATAATACTGCGTCTTGGTTCACCGCTCTCACT | | |
| | | KMKDLSPRWYFYYLGTGPEAGLPY | CAACATGGCAAGGAAGACCTTAAATTCCCTCGAGGACAAGGC | | |
| | | GANKDGIIWVATEGALNTPKDHIGT | GTTCCAATTAACACCAATAGCAGTCCAGATGACCAAATTGGC | | |
| | | RNPANNAAIVLQLPQGTTLPKGFYA | TACTACCGAAGAGCTACCAGACGAATTCGTGGTGGTGACGGT | | |
| | | EGSRGGSQASSRSSSRSRNSSRNSTP | AAAATGAAAGATCTCAGTCCAAGATGGTATTTCTACTACCTA | | |
| | | GSSRGTSPARMAGNGGDAALALLL | GGAACTGGGCCAGAAGCTGGACTTCCCTATGGTGCTAACAAA | | |
| | | LDRLNQLESKMSGKGQQQQGQTV | GACGGCATCATATGGGTTGCAACTGAGGGAGCCTTGAATACA | | |
| | | TKKSAAEASKKPRQKRTATKAYNV | CCAAAAGATCACATTGGCACCCGCAATCCTGCTAACAATGCT | | |
| | | TQAFGRRGPEQTQGNFGDQELIRQ | GCAATCGTGCTACAACTTCCTCAAGGAACAACATTGCCAAAA | | |
| | | GTDYKHWPQIAQFAPSASAFFGMS | GGCTTCTACGCAGAAGGGAGCAGAGGCGGCAGTCAAGCCTCT | | |
| | | RIGMEVTPSGTWLTYTGAIKLDDK | TCTCGTTCCTCATCACGTAGTCGCAACAGTTCAAGAAATTCAA | | |
| | | DPNFKDQVILLNKHIDAYKTFPPTE | CTCCAGGCAGCAGTAGGGGAACTTCTCCTGCTAGAATGGCTG | | |
| | | PKKDKKKKADETQALPQRQKKQQ | GCAATGGCGGTGATGCTGCTCTTGCTTTGCTGCTGCTTGACAG | | |
| | | TVTLLPAADLDDFSKQLQQSMSSA | ATTGAACCAGCTTGAGAGCAAAATGTCTGGTAAAGGCCAACA | | |
| | | DSTQA | ACAACAAGGCCAAACTGTCACTAAGAAATCTGCTGCTGAGGC | | |
| | | | TTCTAAGAAGCCTCGGCAAAAACGTACTGCCACTAAAGCATA | | |
| | | | CAATGTAACACAAGCTTTCGGCAGACGTGGTCCAGAACAAAC | | |
| | | | CCAAGGAAATTTTGGGGACCAGGAACTAATCAGACAAGGAA | | |
| | | | CTGATTACAAACATTGGCCGCAAATTGCACAATTTGCCCCCA | | |
| | | | GCGCTTCAGCGTTCTTCGGAATGTCGCGCATTGGCATGGAAG | | |
| | | | TCACACCTTCGGGAACGTGGTTGACCTACACAGGTGCCATCA | | |
| | | | AATTGGATGACAAAGATCCAAATTTCAAAGATCAAGTCATTT | | |
| | | | TGCTGAATAAGCATATTGACGCATACAAAACATTCCCACCAA | | |
| | | | CAGAGCCTAAAAAGGACAAAAAGAAGAAGGCTGATGAAACT | | |
| | | | CAAGCCTTACCGCAGAGACAGAAGAAACAGCAAACTGTGAC | | |
| | | | TCTTCTTCCTGCTGCAGATTTGGATGATTTCTCCAAACAATTG | | |
| | | | CAACAATCCATGAGCAGTGCTGACTCAACTCAGGCC | | |
| RS C3 2A | 1 GSG | GSGGSGEGRGSLLTCGDVEENPGP | GGCAGCGGCGGCTCCGGCGAGGGCAGGGGAAGTCTTCTAAC | 1363 | 1434 |
| linkers | T2A | | ATGCGGGGACGTGGAGGAAAATCCCGGCCCA | | |
| RS C3 2A | ORF9b | DPKISEMHPALRLVDPQIQLAVTRM | GACCCCAAAATCAGCGAAATGCACCCCGCATTACGTTTGGTG | 1435 | 1722 |
| linkers | protein | ENAVGRDQNNVGPKVYPIILRLGSP | GACCCTCAGATTCAACTGGCAGTAACCAGAATGGAGAACGCA | | |
| | | LSLNMARKTLNSLEDKAFQLTPIAV | GTGGGGCGCGATCAAAACAACGTCGGCCCCAAGGTTTACCCA | | |
| | | QMTKLATTEELPDEFVVVTVK | ATAATACTGCGTCTTGGTTCACCGCTCTCACTCAACATGGCAA | | |
| | | | GGAAGACCTTAAATTCCCTCGAGGACAAGGCGTTCCAATTAA | | |
| | | | CACCAATAGCAGTCCAGATGACCAAATTGGCTACTACCGAAG | | |
| | | | AGCTACCAGACGAATTCGTGGTGGTGACGGTAAAA | | |
| RS C3 2A | 2 GSG | GSGGSGATNFSLLKQAGDVEENPG | GGCAGCGGCGGCAGCGGCGCCACAAACTTCTCTCTGCTAAAG | 1723 | 1797 |
| linkers | P2A | P | CAAGCAGGTGATGTTGAAGAAAACCCCGGGCCT | | |
| RS C3 2A | cleavage | YHFF | TACCATTTCTTT | 1798 | 1809 |
| linkers | linker | | | | |
| RS C3 2A | ORF1ab | HTTDPSFLGRYMSAL | CACACAACTGATCCTAGTTTTCTGGGTAGGTACATGTCAGCAT | 1810 | 1854 |
| linkers | NSP3 | | TA | | |
| | 1636- | | | | |
| | 1650 | | | | |
| RS C3 2A | ORF1ab | FADDLNQLTGY | TTTGCTGATGATTTAAACCAGTTAACTGGTTAT | 1855 | 1887 |
| linkers | NSP3 | | | | |
| | 1937- | | | | |
| | 1947 | | | | |
| RS C3 2A | ORF1ab | HTDFSSEIIGY | CATACTGACTTTTCAAGTGAAATCATAGGATAC | 1888 | 1920 |
| linkers | NSP4 | | | | |
| | 2799- | | | | |
| | 2809 | | | | |
| RS C3 2A | ORF1ab | QLMCQPILL | CAGCTTATGTGTCAACCTATACTGTTA | 1921 | 1947 |
| linkers | NSP3 | | | | |
| | 2563- | | | | |
| | 2571 | | | | |
| RS C3 2A | ORF1ab | AEAELAKNVSL | GCAGAAGCTGAACTTGCAAAGAATGTGTCCTTA | 1948 | 1980 |
| linkers | NSP3 | | | | |
| | 2616- | | | | |
| | 2626 | | | | |
| RS C3 2A | ORF1ab | ILGTVSWNL | ATTCTTGGAACTGTTTCTTGGAATTTG | 1981 | 2007 |
| linkers | NSP3 | | | | |
| | 1367- | | | | |
| | 1375 | | | | |
| RS C3 2A | cleavage | KRRY | AAGCGTCGCTAT | 2008 | 2019 |
| linkers | linker | | | | |
| RS C3 2A | ORF1ab | LLSAGIFGA | TTATTATCAGCTGGTATTTTTGGTGCT | 2020 | 2046 |
| linkers | NSP3 | | | | |
| | 1148- | | | | |
| | 1156 | | | | |
| RS C3 2A | ORF1ab | ITDVFYKENSY | ATTACGGATGTTTTCTACAAAGAAAACAGTTAC | 2047 | 2079 |
| linkers | NSP3 | | | | |
| | 1863- | | | | |
| | 1873 | | | | |
| RS C3 2A | ORF1ab | KVPTDNYITTY | AAAGTGCCAACAGACAATTATATAACCACTTAC | 2080 | 2112 |
| linkers | NSP3 | | | | |
| | 1319- | | | | |
| | 1329 | | | | |
| RS C3 2A | cleavage | ARMA | GCGCGGATGGCT | 2113 | 2124 |
| linkers | linker | | | | |
| RS C3 2A | ORF1ab | APKEIIFLEGETL | GCCCCAAAAGAAATTATCTTCTTAGAGGGAGAAACACTT | 2125 | 2163 |
| linkers | NSP2 | | | | |
| | 735-747 | | | | |
| RS C3 2A | ORF1ab | FGDDTVIEV | TTTGGTGATGACACTGTGATAGAAGTG | 2164 | 2190 |
| linkers | NSP3 | | | | |
| | 825-833 | | | | |
| RS C3 2A | ORF1ab | AIILASFSAST | GCCATTATTTTGGCATCTTTTTCTGCTTCCACA | 2191 | 2223 |
| linkers | NSP2 | | | | |
| | 474-484 | | | | |
| RS C3 2A | cleavage | RRMA | CGCCGTATGGCA | 2224 | 2235 |
| linkers | linker | | | | |
| RS C3 2A | ORF1ab | MVTNNTFTLK | ATGGTAACAAACAATACCTTCACACTCAAA | 2236 | 2265 |
| linkers | NSP2 | | | | |
| | 807-816 | | | | |
| RS C3 2A | ORF1ab | VPHVGEIPVAYRKVLL | GTCCCTCATGTGGGCGAAATACCAGTGGCTTACCGCAAGGTT | 2266 | 2313 |
| linkers | NSP1 | | CTTCTT | | |
| | 108-123 | | | | |
| RS C3 2A | ORF1ab | KTIQPRVEK | AAGACTATTCAACCAAGGGTTGAAAAG | 2314 | 2340 |
| linkers | NSP2 | | | | |
| | 282-290 | | | | |
| RS C3 2A | ORF1ab NSP3 | YLFDESGEFKL | TACTTATTTGATGAGTCTGGTGAGTTTAAATTG | 2341 | 2373 |
| linkers | 906-916 | | | | |
| RS C3 2A | ORF1ab | SEVGPEHSLAEY | TCAGAAGTAGGACCTGAGCATAGTCTTGCCGAATAC | 2374 | 2409 |
| linkers | NSP2 | | | | |
| | 376-387 | | | | |
| RS C3 2A | ORF1ab | YIFFASFYY | TACATCTTCTTTGCATCATTTTATTAT | 2410 | 2436 |
| linkers | NSP3 | | | | |
| | 2384- | | | | |
| | 2392 | | | | |
| RS C3 2A | cleavage | KRNG | AAACGTAACGGG | 2437 | 2448 |
| linkers | linker | | | | |
| RS C3 2A | 3 | GSGVKQTLNFDLLKLAGDVESNPG | GGCAGCGGCGTGAAACAGACTTTGAATTTTGACCTTCTCAAG | 2449 | 2523 |
| linkers | GSG F2A | P | TTGGCGGGAGACGTGGAGTCCAACCCTGGACCT | | |
| RS C3 2A | ORF3a | DLFMRIFTIGTVTLKQGEIKDATPSD | GATTTGTTTATGAGAATCTTCACAATTGGAACTGTAACTTTGA | 2524 | 3345 |
| linkers | protein | FVRATATIPIQASLPFGWLIVGVALL | AGCAAGGTGAAATCAAGGATGCTACTCCTTCAGATTTTGTTC | | |
| | | AVFQSASKIITLKKRWQLALSKGVH | GCGCTACTGCAACGATACCGATACAAGCCTCACTCCCTTTCG | | |
| | | FVCNLLLLFVTVYSHLLLVAAGLEA | GATGGCTTATTGTTGGCGTTGCACTTCTTGCTGTTTTTCAGAG | | |
| | | PFLYLYALVYFLQSINFVRIIMRLWL | CGCTTCCAAAATCATAACCCTCAAAAAGAGATGGCAACTAGC | | |
| | | CWKCRSKNPLLYDANYFLCWHTN | ACTCTCCAAGGGTGTTCACTTTGTTTGCAACTTGCTGTTGTTG | | |
| | | CYDYCIPYNSVTSSIVITSGDGTTSPI | TTTGTAACAGTTTACTCACACCTTTTGCTCGTTGCTGCTGGCCT | | |
| | | SEHDYQIGGYTEKWESGVKDCVVL | TGAAGCCCCTTTTCTCTATCTTTATGCTTTAGTCTACTTCTTGC | | |
| | | HSYFTSDYYQLYSTQLSTDTGVEH | AGAGTATAAACTTTGTAAGAATAATAATGAGGCTTTGGCTTT | | |
| | | VTFFIYNKIVDEPEEHVQIHTIDGSS | GCTGGAAATGCCGTTCCAAAAACCCATTACTTTATGATGCCA | | |
| | | GVVNPVMEPIYDEPTTTTSVPL | ACTATTTTCTTTGCTGGCATACTAATTGTTACGACTATTGTAT | | |
| | | | ACCTTACAATAGTGTAACTTCTTCAATTGTCATTACTTCAGGT | | |
| | | | GATGGCACAACAAGTCCTATTTCTGAACATGACTACCAGATT | | |
| | | | GGTGGTTATACTGAAAAATGGGAATCTGGAGTAAAAGACTGT | | |
| | | | GTTGTATTACACAGTTACTTCACTTCAGACTATTACCAGCTGT | | |
| | | | ACTCAACTCAATTGAGTACAGACACTGGTGTTGAACATGTTA | | |
| | | | CCTTCTTCATCTACAATAAAATTGTTGATGAGCCTGAAGAACA | | |
| | | | TGTCCAAATTCACACAATCGACGGTTCATCCGGAGTTGTTAAT | | |
| | | | CCAGTAATGGAACCAATTTATGATGAACCGACGACGACTACT | | |
| | | | AGCGTGCCTTTG | | |
| RS C3 2A | 4 GSG | GSGGSGQCTNYALLKLAGDVESNP | GGCAGCGGCGGCTCGGGCCAGTGTACTAATTATGCTCTCTTG | 3346 | 3423 |
| linkers | E2A | GP | AAATTGGCTGGAGATGTTGAGAGCAACCCAGGTCCC | | |
| RS C3 2A | membrane | ADSNGTITVEELKKLLEQWNLVIGF | GCAGATTCCAACGGTACTATTACCGTTGAAGAGCTTAAAAAG | 3424 | 4086 |
| linkers | glycoprotein | LFLTWICLLQFAYANRNRFLYIIKLI | CTCCTTGAACAATGGAACCTAGTAATAGGTTTCCTATTCCTTA | | |
| | | FLWLLWPVTLACFVLAAVYRINWI | CATGGATTTGTCTTCTACAATTTGCCTATGCCAACAGGAATAG | | |
| | | TGGIAIAMACLVGLMWLSYFIASFR | GTTTTTGTATATAATTAAGTTAATTTTCCTCTGGCTGTTATGGC | | |
| | | LFARTRSMWSFNPETNILLNVPLHG | CAGTAACTTTAGCTTGTTTTGTGCTTGCTGCTGTTTACAGAAT | | |
| | | TILTRPLLESELVIGAVILRGHLRIAG | AAATTGGATCACCGGTGGAATTGCTATCGCAATGGCTTGTCTT | | |
| | | HHLGRCDIKDLPKEITVATSRTLSY | GTAGGCTTGATGTGGCTCAGCTACTTCATTGCTTCTTTCAGAC | | |
| | | YKLGASQRVAGDSGFAAYSRYRIG | TGTTTGCGCGTACGCGTTCCATGTGGTCATTCAATCCAGAAAC | | |
| | | NYKLNTDHSSSSDNIALLVQ | TAACATTCTTCTCAACGTGCCACTCCATGGCACTATTCTGACC | | |
| | | | AGACCGCTTCTAGAAAGTGAACTCGTAATCGGAGCTGTGATC | | |
| | | | CTTCGTGGACATCTTCGTATTGCTGGACACCATCTAGGACGCT | | |
| | | | GTGACATCAAGGACCTGCCTAAAGAAATCACTGTTGCTACAT | | |
| | | | CACGAACGCTTTCTTATTACAAATTGGGAGCTTCGCAGCGTGT | | |
| | | | AGCAGGTGACTCAGGTTTTGCTGCATACAGTCGCTACAGGAT | | |
| | | | TGGCAACTATAAATTAAACACAGACCATTCCAGTAGCAGTGA | | |
| | | | CAATATTGCTTTGCTTGTACAG | | |
| RS C3 2A | end GSS | GGSLGGGGSG | GGTGGTTCTCTTGGCGGAGGGGGTTCGGGA | 4087 | 4116 |
| linkers | | | | | |
| RS C3 2A | MITD | IVGIVAGLAVLAVVVIGAVVATVM | ATCGTGGGAATTGTGGCAGGACTGGCAGTGCTGGCCGTGGTG | 4117 | 4287 |
| linkers | | CRRKSSGGKGGSYSQAASSDSAQG | GTGATCGGAGCCGTGGTGGCTACCGTGATGTGCAGACGGAAG | | |
| | | SDVSLTA** | TCCAGCGGAGGCAAGGGCGGCAGCTACAGCCAGGCCGCCAG | | |
| | | | CTCTGATAGCGCCCAGGGCAGCGACGTGTCACTGACAGCCTA | | |
| | | | GTAA | | |
| RS C4 | SP1 | MRVMAPRTLILLLSGALALTETWA | ATGAGAGTGATGGCCCCCAGAACCCTGATCCTGCTGCTGTCT | 1 | 78 |
| ORF1ab as | | GS | GGCGCCCTGGCCCTGACAGAGACATGGGCCGGAAGC | | |
| linkers | | | | | |
| RS C4 | start GSS | GGSGGGGSGG | GGAGGTTCCGGAGGAGGCGGCAGTGGCGGC | 79 | 108 |
| ORF1ab as | | | | | |
| linkers | | | | | |
| RS C4 | nucleocapsid | SDNGPQNQRNAPRITFGGPSDSTGS | TCTGATAATGGACCCCAAAATCAGCGAAATGCACCCCGCATT | 109 | 1362 |
| ORF1ab as | phosphoprotein | NONGERSGARSKQRRPQGLPNNTA | ACGTTTGGTGGACCCTCAGATTCAACTGGCAGTAACCAGAAT | | |
| linkers | | SWFTALTQHGKEDLKFPRGQGVPI | GGAGAACGCAGTGGGGCGCGATCAAAACAACGTCGGCCCCA | | |
| | | NTNSSPDDQIGYYRRATRRIRGGDG | AGGTTTACCCAATAATACTGCGTCTTGGTTCACCGCTCTCACT | | |
| | | KMKDLSPRWYFYYLGTGPEAGLPY | CAACATGGCAAGGAAGACCTTAAATTCCCTCGAGGACAAGGC | | |
| | | GANKDGIIWVATEGALNTPKDHIGT | GTTCCAATTAACACCAATAGCAGTCCAGATGACCAAATTGGC | | |
| | | RNPANNAAIVLQLPQGTTLPKGFYA | TACTACCGAAGAGCTACCAGACGAATTCGTGGTGGTGACGGT | | |
| | | EGSRGGSQASSRSSSRSRNSSRNSTP | AAAATGAAAGATCTCAGTCCAAGATGGTATTTCTACTACCTA | | |
| | | GSSRGTSPARMAGNGGDAALALLL | GGAACTGGGCCAGAAGCTGGACTTCCCTATGGTGCTAACAAA | | |
| | | LDRLNQLESKMSGKGQQQQGQTV | GACGGCATCATATGGGTTGCAACTGAGGGAGCCTTGAATACA | | |
| | | TKKSAAEASKKPRQKRTATKAYNV | CCAAAAGATCACATTGGCACCCGCAATCCTGCTAACAATGCT | | |
| | | TQAFGRRGPEQTQGNFGDQELIRQ | GCAATCGTGCTACAACTTCCTCAAGGAACAACATTGCCAAAA | | |
| | | GTDYKHWPQIAQFAPSASAFFGMS | GGCTTCTACGCAGAAGGGAGCAGAGGCGGCAGTCAAGCCTCT | | |
| | | RIGMEVTPSGTWLTYTGAIKLDDK | TCTCGTTCCTCATCACGTAGTCGCAACAGTTCAAGAAATTCAA | | |
| | | DPNFKDQVILLNKHIDAYKTFPPTE | CTCCAGGCAGCAGTAGGGGAACTTCTCCTGCTAGAATGGCTG | | |
| | | PKKDKKKKADETQALPQRQKKQQ | GCAATGGCGGTGATGCTGCTCTTGCTTTGCTGCTGCTTGACAG | | |
| | | TVTLLPAADLDDFSKQLQQSMSSA | ATTGAACCAGCTTGAGAGCAAAATGTCTGGTAAAGGCCAACA | | |
| | | DSTQA | ACAACAAGGCCAAACTGTCACTAAGAAATCTGCTGCTGAGGC | | |
| | | | TTCTAAGAAGCCTCGGCAAAAACGTACTGCCACTAAAGCATA | | |
| | | | CAATGTAACACAAGCTTTCGGCAGACGTGGTCCAGAACAAAC | | |
| | | | CCAAGGAAATTTTGGGGACCAGGAACTAATCAGACAAGGAA | | |
| | | | CTGATTACAAACATTGGCCGCAAATTGCACAATTTGCCCCCA | | |
| | | | GCGCTTCAGCGTTCTTCGGAATGTCGCGCATTGGCATGGAAG | | |
| | | | TCACACCTTCGGGAACGTGGTTGACCTACACAGGTGCCATCA | | |
| | | | AATTGGATGACAAAGATCCAAATTTCAAAGATCAAGTCATTT | | |
| | | | TGCTGAATAAGCATATTGACGCATACAAAACATTCCCACCAA | | |
| | | | CAGAGCCTAAAAAGGACAAAAAGAAGAAGGCTGATGAAACT | | |
| | | | CAAGCCTTACCGCAGAGACAGAAGAAACAGCAAACTGTGAC | | |
| | | | TCTTCTTCCTGCTGCAGATTTGGATGATTTCTCCAAACAATTG | | |
| | | | CAACAATCCATGAGCAGTGCTGACTCAACTCAGGCC | | |
| RS C4 | cleavage | FRAC | TTCAGGGCTTGC | 1363 | 1374 |
| ORF1ab as | linker | | | | |
| linkers | | | | | |
| RS C4 | ORF1ab | MVTNNTFTLK | ATGGTAACAAACAATACCTTCACACTCAAA | 1375 | 1404 |
| ORFlab as | NSP2 | | | | |
| linkers | 807-816 | | | | |
| RS C4 | ORF1ab | VPHVGEIPVAYRKVLL | GTCCCTCATGTGGGCGAAATACCAGTGGCTTACCGCAAGGTT | 1405 | 1452 |
| ORFlab as | NSP1 | | CTTCTT | | |
| linkers | 108-123 | | | | |
| RS C4 | ORF1ab | KTIQPRVEK | AAGACTATTCAACCAAGGGTTGAAAAG | 1453 | 1479 |
| ORF1ab as | NSP2 | | | | |
| linkers | 282-290 | | | | |
| RS C4 | ORF1ab | YLFDESGEFKL | TACTTATTTGATGAGTCTGGTGAGTTTAAATTG | 1480 | 1512 |
| ORF1ab as | NSP3 | | | | |
| linkers | 906-916 | | | | |
| RS C4 | ORF1ab | SEVGPEHSLAEY | TCAGAAGTAGGACCTGAGCATAGTCTTGCCGAATAC | 1513 | 1548 |
| ORF1ab as | NSP2 | | | | |
| linkers | 376-387 | | | | |
| RS C4 | ORF1ab | YIFFASFYY | TACATCTTCTTTGCATCATTTTATTAT | 1549 | 1575 |
| ORF1ab | NSP3 | | | | |
| as | 2384- | | | | |
| linkers | 2392 | | | | |
| RS C4 | cleavage | KRNG | AAAAGAAATGGC | 1576 | 1587 |
| ORFlab as | linker | | | | |
| linkers | | | | | |
| RS C4 | ORF9b | DPKISEMHPALRLVDPQIQLAVTRM | GACCCCAAAATCAGCGAAATGCACCCCGCATTACGTTTGGTG | 1588 | 1875 |
| ORF1ab as | protein | ENAVGRDQNNVGPKVYPIILRLGSP | GACCCTCAGATTCAACTGGCAGTAACCAGAATGGAGAACGCA | | |
| linkers | | LSLNMARKTLNSLEDKAFQLTPIAV | GTGGGGCGCGATCAAAACAACGTCGGCCCCAAGGTTTACCCA | | |
| | | QMTKLATTEELPDEFVVVTVK | ATAATACTGCGTCTTGGTTCACCGCTCTCACTCAACATGGCAA | | |
| | | | GGAAGACCTTAAATTCCCTCGAGGACAAGGCGTTCCAATTAA | | |
| | | | CACCAATAGCAGTCCAGATGACCAAATTGGCTACTACCGAAG | | |
| | | | AGCTACCAGACGAATTCGTGGTGGTGACGGTAAAA | | |
| RS C4 | cleavage | FNSF | TTCAACTCTTTT | 1876 | 1887 |
| ORF1ab as | linker | | | | |
| linkers | | | | | |
| RS C4 | ORF1ab | HTTDPSFLGRYMSAL | CACACAACTGATCCTAGTTTTCTGGGTAGGTACATGTCAGCAT | 1888 | 1932 |
| ORF1ab | NSP3 | | TA | | |
| as | 1636- | | | | |
| linkers | 1650 | | | | |
| RS C4 | ORF1ab | FADDLNQLTGY | TTTGCTGATGATTTAAACCAGTTAACTGGTTAT | 1933 | 1965 |
| ORF1ab as | NSP3 | | | | |
| linkers | 1937- | | | | |
| | 1947 | | | | |
| RS C4 | ORF1ab | HTDFSSEIIGY | CATACTGACTTTTCAAGTGAAATCATAGGATAC | 1966 | 1998 |
| ORF1ab | NSP4 | | | | |
| as | 2799- | | | | |
| linkers | 2809 | | | | |
| RS C4 | ORF1ab | QLMCQPILL | CAGCTTATGTGTCAACCTATACTGTTA | 1999 | 2025 |
| ORF1ab | INSP3 | | | | |
| as | 2563- | | | | |
| linkers | 2571 | | | | |
| RS C4 | ORF1ab | AEAELAKNVSL | GCAGAAGCTGAACTTGCAAAGAATGTGTCCTTA | 2026 | 2058 |
| ORF1ab | NSP3 | | | | |
| as | 2616- | | | | |
| linkers | 2626 | | | | |
| RS C4 | ORF1ab | ILGTVSWNL | ATTCTTGGAACTGTTTCTTGGAATTTG | 2059 | 2085 |
| ORF1ab | NSP3 | | | | |
| as | 1367- | | | | |
| linkers | 1375 | | | | |
| RS C4 | cleavage | KKQG | AAAAAACAGGGC | 2086 | 2097 |
| ORF1ab as | linker | | | | |
| linkers | | | | | |
| RS C4 | ORF3a | DLFMRIFTIGTVTLKQGEIKDATPSD | ACCTTACAATAGTGTAACTTCTTCAATTGTCATTACTTCAGGT | 2098 | 2919 |
| ORF1ab as | protein | FVRATATIPIQASLPFGWLIVGVALL | GGTGGTTATACTGAAAAATGGGAATCTGGAGTAAAAGACTGT | | |
| linkers | | AVFQSASKIITLKKRWQLALSKGVH | GATTTGTTTATGAGAATCTTCACAATTGGAACTGTAACTTTGA | | |
| | | FVCNLLLLFVTVYSHLLLVAAGLEA | AGCAAGGTGAAATCAAGGATGCTACTCCTTCAGATTTTGTTC | | |
| | | PFLYLYALVYFLQSINFVRIIMRLWL | GCGCTACTGCAACGATACCGATACAAGCCTCACTCCCTTTCG | | |
| | | CWKCRSKNPLLYDANYFLCWHTN | GATGGCTTATTGTTGGCGTTGCACTTCTTGCTGTTTTTCAGAG | | |
| | | CYDYCIPYNSVTSSIVITSGDGTTSPI | CGCTTCCAAAATCATAACCCTCAAAAAGAGATGGCAACTAGC | | |
| | | SEHDYQIGGYTEKWESGVKDCVVL | ACTCTCCAAGGGTGTTCACTTTGTTTGCAACTTGCTGTTGTTG | | |
| | | HSYFTSDYYQLYSTQLSTDTGVEH | TTTGTAACAGTTTACTCACACCTTTTGCTCGTTGCTGCTGGCCT | | |
| | | VTFFIYNKIVDEPEEHVQIHTIDGSS | TGAAGCCCCTTTTCTCTATCTTTATGCTTTAGTCTACTTCTTGC | | |
| | | GVVNPVMEPIYDEPTTTTSVPL | AGAGTATAAACTTTGTAAGAATAATAATGAGGCTTTGGCTTT | | |
| | | | GCTGGAAATGCCGTTCCAAAAACCCATTACTTTATGATGCCA | | |
| | | | ACTATTTTCTTTGCTGGCATACTAATTGTTACGACTATTGTAT | | |
| | | | GATGGCACAACAAGTCCTATTTCTGAACATGACTACCAGATT | | |
| | | | GTTGTATTACACAGTTACTTCACTTCAGACTATTACCAGCTGT | | |
| | | | ACTCAACTCAATTGAGTACAGACACTGGTGTTGAACATGTTA | | |
| | | | CCTTCTTCATCTACAATAAAATTGTTGATGAGCCTGAAGAACA | | |
| | | | TGTCCAAATTCACACAATCGACGGTTCATCCGGAGTTGTTAAT | | |
| | | | CCAGTAATGGAACCAATTTATGATGAACCGACGACGACTACT | | |
| | | | AGCGTGCCTTTG | | |
| RS C4 | cleavage | RRSY | CGCCGGAGTTAC | 2920 | 2931 |
| ORF1ab as | linker | | | | |
| linkers | | | | | |
| RS C4 | ORF1ab | LLSAGIFGA | TTATTATCAGCTGGTATTTTTGGTGCT | 2932 | 2958 |
| ORF1ab as | NSP3 | | | | |
| linkers | 1148- | | | | |
| | 1156 | | | | |
| RS C4 | ORF1ab | ITDVFYKENSY | ATTACGGATGTTTTCTACAAAGAAAACAGTTAC | 2959 | 2991 |
| ORF1ab | NSP3 | | | | |
| as | 1863- | | | | |
| linkers | 1873 | | | | |
| RS C4 | ORF1ab | KVPTDNYITTY | AAAGTGCCAACAGACAATTATATAACCACTTAC | 2992 | 3024 |
| ORF1ab as | NSP3 | | | | |
| linkers | 1319- | | | | |
| | 1329 | | | | |
| RS C4 | cleavage | ARMA | GCGCGAATGGCG | 3025 | 3036 |
| ORFlab as | linker | | | | |
| linkers | | | | | |
| RS C4 | ORF1ab | APKEIIFLEGETL | GCCCCAAAAGAAATTATCTTCTTAGAGGGAGAAACACTT | 3037 | 3075 |
| ORF1ab as | NSP2 | | | | |
| linkers | 735-747 | | | | |
| RS C4 | ORF1ab | FGDDTVIEV | TTTGGTGATGACACTGTGATAGAAGTG | 3076 | 3102 |
| ORFlab as | NSP3 | | | | |
| linkers | 825-833 | | | | |
| RS C4 | ORF1ab | AIILASFSAST | GCCATTATTTTGGCATCTTTTTCTGCTTCCACA | 3103 | 3135 |
| ORFlab as | NSP2 474-484 | | | | |
| linkers | | | | | |
| RS C4 | cleavage | LLVQ | CTGCTGGTCCAG | 3136 | 3147 |
| ORF1ab as | linker | | | | |
| linkers | | | | | |
| RS C4 | membrane | ADSNGTITVEELKKLLEQWNLVIGF | GCAGATTCCAACGGTACTATTACCGTTGAAGAGCTTAAAAAG | 3148 | 3810 |
| ORFlab as | glycoprotein | LFLTWICLLQFAYANRNRFLYIIKLI | CTCCTTGAACAATGGAACCTAGTAATAGGTTTCCTATTCCTTA | | |
| linkers | | FLWLLWPVTLACFVLAAVYRINWI | CATGGATTTGTCTTCTACAATTTGCCTATGCCAACAGGAATAG | | |
| | | TGGIAIAMACLVGLMWLSYFIASFR | GTTTTTGTATATAATTAAGTTAATTTTCCTCTGGCTGTTATGGC | | |
| | | LFARTRSMWSFNPETNILLNVPLHG | CAGTAACTTTAGCTTGTTTTGTGCTTGCTGCTGTTTACAGAAT | | |
| | | TILTRPLLESELVIGAVILRGHLRIAG | AAATTGGATCACCGGTGGAATTGCTATCGCAATGGCTTGTCTT | | |
| | | HHLGRCDIKDLPKEITVATSRTLSY | GTAGGCTTGATGTGGCTCAGCTACTTCATTGCTTCTTTCAGAC | | |
| | | YKLGASQRVAGDSGFAAYSRYRIG | TGTTTGCGCGTACGCGTTCCATGTGGTCATTCAATCCAGAAAC | | |
| | | NYKLNTDHSSSSDNIALLVQ | TAACATTCTTCTCAACGTGCCACTCCATGGCACTATTCTGACC | | |
| | | | AGACCGCTTCTAGAAAGTGAACTCGTAATCGGAGCTGTGATC | | |
| | | | CTTCGTGGACATCTTCGTATTGCTGGACACCATCTAGGACGCT | | |
| | | | GTGACATCAAGGACCTGCCTAAAGAAATCACTGTTGCTACAT | | |
| | | | CACGAACGCTTTCTTATTACAAATTGGGAGCTTCGCAGCGTGT | | |
| | | | AGCAGGTGACTCAGGTTTTGCTGCATACAGTCGCTACAGGAT | | |
| | | | TGGCAACTATAAATTAAACACAGACCATTCCAGTAGCAGTGA | | |
| | | | CAATATTGCTTTGCTTGTACAG | | |
| RS C4 | end GSS | GGSLGGGGSG | GGTGGTTCTCTTGGCGGAGGGGGTTCGGGA | 3811 | 3840 |
| ORF1ab as | | | | | |
| linkers | | | | | |
| RS C4 | MITD | IVGIVAGLAVLAVVVIGAVVATVM | ATCGTGGGAATTGTGGCAGGACTGGCAGTGCTGGCCGTGGTG | 3841 | 4011 |
| ORF1ab as | | CRRKSSGGKGGSYSQAASSDSAQG | GTGATCGGAGCCGTGGTGGCTACCGTGATGTGCAGACGGAAG | | |
| linkers | | SDVSLTA** | TCCAGCGGAGGCAAGGGCGGCAGCTACAGCCAGGCCGCCAG | | |
| | | | CTCTGATAGCGCCCAGGGCAGCGACGTGTCACTGACAGCCTA | | |
| | | | GTAA | | |

TABLE 10
Group 2 Construct-maps

| CONSTRUCT | Segment | | | | |
|---|---|---|---|---|---|
| Name | NAME | AA SEQUENCE | DNA SEQUENCE | START | END |
| RS C5 2300 | SP1 | MRVMAPRTLILLLSGALALTETWAG | ATGAGAGTGATGGCCCCCAGAACCC | 1 | 78 |
| | | S | TGATCCTGCTGCTGTCTGGCGCCCT | | |
| | | | GGCCCTGACAGAGACATGGGCCGGA | | |
| | | | AGC | | |
| RS C5 2300 | start GSS | GGSGGGGSGG | GGAGGTTCCGGAGGAGGCGGCAGTG | 79 | 108 |
| | | | GCGGC | | |
| RS C5 2300 | N without | KDLSPRWYFYYLGTGPEAGLPYGAN | GAGGCTTCTAAGAAGCCTCGGCAAA | 109 | 1062 |
| | Orf9b | KDGIIWVATEGALNTPKDHIGTRNP | AACGTACTGCCACTAAAAAGATCTC | | |
| | | ANNAAIVLQLPQGTTLPKGFYAEGS | AGTCCAAGATGGTATTTCTACTACC | | |
| | | RGGSQASSRSSSRSRNSSRNSTPGS | TAGGAACTGGGCCAGAAGCTGGACT | | |
| | | SRGTSPARMAGNGGDAALALLLLDR | TCCCTATGGTGCTAACAAAGACGGC | | |
| | | LNQLESKMSGKGQQQQGQTVTKKSA | ATCATATGGGTTGCAACTGAGGGAG | | |
| | | AEASKKPRQKRTATKAYNVTQAFGR | CCTTGAATACACCAAAAGATCACAT | | |
| | | RGPEQTQGNFGDQELIRQGTDYKHW | TGGCACCCGCAATCCTGCTAACAAT | | |
| | | PQIAQFAPSASAFFGMSRIGMEVTP | GCTGCAATCGTGCTACAACTTCCTC | | |
| | | SGTWLTYTGAIKLDDKDPNFKDQVI | AAGGAACAACATTGCCAAAAGGCTT | | |
| | | LLNKHIDAYKTFPPTEPKKDKKKKA | CTACGCAGAAGGGAGCAGAGGCGGC | | |
| | | DETQALPQRQKKQQTVTLLPAADLD | AGTCAAGCCTCTTCTCGTTCCTCAT | | |
| | | DFSKQLQQSMSSADSTQA | CACGTAGTCGCAACAGTTCAAGAAA | | |
| | | | TTCAACTCCAGGCAGCAGTAGGGGA | | |
| | | | ACTTCTCCTGCTAGAATGGCTGGCA | | |
| | | | ATGGCGGTGATGCTGCTCTTGCTTT | | |
| | | | GCTGCTGCTTGACAGATTGAACCAG | | |
| | | | CTTGAGAGCAAAATGTCTGGTAAAG | | |
| | | | GCCAACAACAACAAGGCCAAACTGT | | |
| | | | CACTAAGAAATCTGCTGCTAGCATA | | |
| | | | CAATGTAACACAAGCTTTCGGCAGA | | |
| | | | CGTGGTCCAGAACAAACCCAAGGAA | | |
| | | | ATTTTGGGGACCAGGAACTAATCAG | | |
| | | | ACAAGGAACTGATTACAAACATTGG | | |
| | | | CCGCAAATTGCACAATTTGCCCCCA | | |
| | | | GCGCTTCAGCGTTCTTCGGAATGTC | | |
| | | | GCGCATTGGCATGGAAGTCACACCT | | |
| | | | TCGGGAACGTGGTTGACCTACACAG | | |
| | | | GTGCCATCAAATTGGATGACAAAGA | | |
| | | | TCCAAATTTCAAAGATCAAGTCATT | | |
| | | | TTGCTGAATAAGCATATTGACGCAT | | |
| | | | ACAAAACATTCCCACCAACAGAGCC | | |
| | | | TAAAAAGGACAAAAAGAAGAAGGCT | | |
| | | | GATGAAACTCAAGCCTTACCGCAGA | | |
| | | | GACAGAAGAAACAGCAAACTGTGAC | | |
| | | | TCTTCTTCCTGCTGCAGATTTGGAT | | |
| | | | GATTTCTCCAAACAATTGCAACAAT | | |
| | | | CCATGAGCAGTGCTGACTCAACTCA | | |
| | | | GGCC | | |
| RS C5 2300 | cleavage | FRAC | TTCCGCGCGTGC | 1063 | 1074 |
| | linker | | | | |
| RS C5 2300 | 807-816 | MVTNNTFTLK | ATGGTAACAAACAATACCTTCACAC | 1075 | 1104 |
| | ORF1ab | | TCAAA | | |
| | NSP2 | | | | |
| RS C5 2300 | ORF1ab | VPHVGEIPVAY | GTCCCTCATGTGGGCGAAATACCAG | 1105 | 1152 |
| | NSP1 | RKVLL | TGGCTTACCGCAAGGTTCTTCTT | | |
| | 108-123 | | | | |
| RS C5 2300 | ORF1ab | KTIQPRVEK | AAGACTATTCAACCAAGGGTTGAAA | 1153 | 1179 |
| | NSP2 | | AG | | |
| | 282-290 | | | | |
| RS C5 2300 | ORF1ab | YLFDESGEFKL | TACTTATTTGATGAGTCTGGTGAGT | 1180 | 1212 |
| | NSP3 | | TTAAATTG | | |
| | 906-916 | | | | |
| RS C5 2300 | ORF1ab | SEVGPEHSLAEY | TCAGAAGTAGGACCTGAGCATAGTC | 1213 | 1248 |
| | NSP2 | | TTGCCGAATAC | | |
| | 376-387 | | | | |
| RS C5 2300 | ORF1ab | YIFFASFYY | TACATCTTCTTTGCATCATTTTATT | 1249 | 1275 |
| | NSP3 | | AT | | |
| | 2384- | | | | |
| | 2392 | | | | |
| RS C5 2300 | cleavage | KRCF | AAAAGATGTTTT | 1276 | 1287 |
| | linker | | | | |
| RS C5 2300 | ORF1ab | HTTDPSFLGRYMSAL | CACACAACTGATCCTAGTTTTCTGG | 1288 | 1332 |
| | NSP3 | | GTAGGTACATGTCAGCATTA | | |
| | 1636- | | | | |
| | 1650 | | | | |
| RS C5 2300 | ORF1ab | FADDLNQLTGY | TTTGCTGATGATTTAAACCAGTTAA | 1333 | 1365 |
| | NSP3 | | CTGGTTAT | | |
| | 1937- | | | | |
| | 1947 | | | | |
| | ORF1ab | HTDFSSEIIGY | CATACTGACTTTTCAAGTGAAATCA | 1366 | 1398 |
| RS C5 2300 | NSP4 | | TAGGATAC | | |
| | 2799- | | | | |
| | 2809 | | | | |
| RS C5 2300 | ORF1ab | QLMCQPILL | CAGCTTATGTGTCAACCTATACTGT | 1399 | 1425 |
| | NSP3 | | TA | | |
| | 2563- | | | | |
| | 2571 | | | | |
| RS C5 2300 | ORF1ab | AEAELAKNVSL | GCAGAAGCTGAACTTGCAAAGAATG | 1426 | 1458 |
| | NSP3 | | TGTCCTTA | | |
| | 2616- | | | | |
| | 2626 | | | | |
| RS C5 2300 | ORF1ab | ILGTVSWNL | ATTCTTGGAACTGTTTCTTGGAATT | 1459 | 1485 |
| | NSP3 | | TG | | |
| | 1367- | | | | |
| | 1375 | | | | |
| RS C5 2300 | cleavage | KKRY | AAAAAGCGTTAT | 1486 | 1497 |
| | linker | | | | |
| RS C5 2300 | ORF1ab | LLSAGIFGA | TTATTATCAGCTGGTATTTTTGGTG | 1498 | 1524 |
| | NSP3 | | CT | | |
| | 1148- | | | | |
| | 1156 | | | | |
| RS C5 2300 | ORF1ab | ITDVFYKENSY | ATTACGGATGTTTTCTACAAAGAAA | 1525 | 1557 |
| | NSP3 | | ACAGTTAC | | |
| | 1863- | | | | |
| | 1873 | | | | |
| RS C5 2300 | ORF1ab | KVPTDNYITTY | AAAGTGCCAACAGACAATTATATAA | 1558 | 1590 |
| | NSP3 | | CCACTTAC | | |
| | 1319- | | | | |
| | 1329 | | | | |
| RS C5 2300 | cleavage | ARMA | GCCCGAATGGCT | 1591 | 1602 |
| | linker | | | | |
| RS C5 2300 | ORF1ab | APKEIIFLEGETL | GCCCCAAAAGAAATTATCTTCTTAG | 1603 | 1641 |
| | NSP2 | | AGGGAGAAACACTT | | |
| | 735-747 | | | | |
| RS C5 2300 | ORF1ab | FGDDTVIEV | TTTGGTGATGACACTGTGATAGAAG | 1642 | 1668 |
| | NSP3 | | TG | | |
| | 825-833 | | | | |
| RS C5 2300 | ORF1ab | AIILASFSAST | GCCATTATTTTGGCATCTTTTTCTG | 1669 | 1701 |
| | NSP2 | | CTTCCACA | | |
| | 474-484 | | | | |
| RS C5 2300 | cleavage | RRSG | AGGAGGTCTGGA | 1702 | 1713 |
| | linker | | | | |
| RS C5 2300 | membrane | ADSNGTITVEELKKLLEQWNLVIGF | GCAGATTCCAACGGTACTATTACCG | 1714 | 2376 |
| | glycoprotein | LFLTWICLLQFAYANRNRFLYIIKL | TTGAAGAGCTTAAAAAGCTCCTTGA | | |
| | | IFLWLLWPVTLACFVLAAVYRINWI | ACAATGGAACCTAGTAATAGGTTTC | | |
| | | TGGIAIAMACLVGLMWLSYFIASFR | CTATTCCTTACATGGATTTGTCTTC | | |
| | | LFARTRSMWSFNPETNILLNVPLHG | TACAATTTGCCTATGCCAACAGGAA | | |
| | | TILTRPLLESELVIGAVILRGHLRI | TAGGTTTTTGTATATAATTAAGTTA | | |
| | | AGHHLGRCDIKDLPKEITVATSRTL | ATTTTCCTCTGGCTGTTATGGCCAG | | |
| | | SYYKLGASQRVAGDSGFAAYSRYRI | TAACTTTAGCTTGTTTTGTGCTTGC | | |
| | | GNYKLNTDHSSSSDNIALLVQ | TGCTGTTTACAGAATAAATTGGATC | | |
| | | | ACCGGTGGAATTGCTATCGCAATGG | | |
| | | | CTTGTCTTGTAGGCTTGATGTGGCT | | |
| | | | CAGCTACTTCATTGCTTCTTTCAGA | | |
| | | | CTGTTTGCGCGTACGCGTTCCATGT | | |
| | | | GGTCATTCAATCCAGAAACTAACAT | | |
| | | | TCTTCTCAACGTGCCACTCCATGGC | | |
| | | | ACTATTCTGACCAGACCGCTTCTAG | | |
| | | | AAAGTGAACTCGTAATCGGAGCTGT | | |
| | | | GATCCTTCGTGGACATCTTCGTATT | | |
| | | | GCTGGACACCATCTAGGACGCTGTG | | |
| | | | ACATCAAGGACCTGCCTAAAGAAAT | | |
| | | | CACTGTTGCTACATCACGAACGCTT | | |
| | | | TCTTATTACAAATTGGGAGCTTCGC | | |
| | | | AGCGTGTAGCAGGTGACTCAGGTTT | | |
| | | | TGCTGCATACAGTCGCTACAGGATT | | |
| | | | GGCAACTATAAATTAAACACAGACC | | |
| | | | ATTCCAGTAGCAGTGACAATATTGC | | |
| | | | TTTGCTTGTACAG | | |
| RS C5 2300 | end GSS | GGSLGGGGSG | GGTGGTTCTCTTGGCGGAGGGGGTT | 2377 | 2406 |
| | | | CGGGA | | |
| RS C5 2300 | MITD | IVGIVAGLAVLAVVVIGAVVATVMC | ATCGTGGGAATTGTGGCAGGACTGG | 2407 | 2577 |
| | | RRKSSGGKGGSYSQAASSDSAQGSD | CAGTGCTGGCCGTGGTGGTGATCGG | | |
| | | VSLTA** | AGCCGTGGTGGCTACCGTGATGTGC | | |
| | | | AGACGGAAGTCCAGCGGAGGCAAGG | | |
| | | | GCGGCAGCTACAGCCAGGCCGCCAG | | |
| | | | CTCTGATAGCGCCCAGGGCAGCGAC | | |
| | | | GTGTCACTGACAGCCTAGTAA | | |
| RS C6 1200 | SP1 | MRVMAPRTLILLLSGALALTETWAG | ATGAGAGTGATGGCCCCCAGAACCC | 1 | 78 |
| | | IS | TGATCCTGCTGCTGTCTGGCGCCCT | | |
| | | | GGCCCTGACAGAGACATGGGCCGGA | | |
| | | | AGC | | |
| RS C6 1200 | start GSS | GGSGGGGSGG | GGAGGTTCCGGAGGAGGCGGCAGTG | 79 | 108 |
| | | | GCGGC | | |
| RS C6 1200 | N 99 | RMAGNGGDAALALLLLDRLNQLESK | AGAATGGCTGGCAATGGCGGTGATG | 109 | 207 |
| | | MSGKGQQQ | CTGCTCTTGCTTTGCTGCTGCTTGA | | |
| | | | CAGATTGAACCAGCTTGAGAGCAAA | | |
| | | | ATGTCTGGTAAAGGCCAACAACAA | | |
| RS C6 1200 | cleavage | MRAC | ATGAGAGCGTGC | 208 | 219 |
| | linker | | | | |
| RS C6 1200 | ORF1ab | MVTNNTFTLK | ATGGTAACAAACAATACCTTCACAC | 220 | 249 |
| | NSP2 | | TCAAA | | |
| | 807-816 | | | | |
| RS C6 1200 | ORF1ab | VPHVGEIPVAYRKVLL | GTCCCTCATGTGGGCGAAATACCAG | 250 | 297 |
| | NSP1 | | TGGCTTACCGCAAGGTTCTTCTT | | |
| | 108-123 | | | | |
| RS C6 1200 | ORF1ab | KTIQPRVEK | AAGACTATTCAACCAAGGGTTGAAA | 298 | 324 |
| | NSP2 | | AG | | |
| | 282-290 | | | | |
| RS C6 1200 | ORF1ab | YLFDESGEFKL | TACTTATTTGATGAGTCTGGTGAGT | 325 | 357 |
| | NSP3 | | TTAAATTG | | |
| | 906-916 | | | | |
| RS C6 1200 | ORF1ab | SEVGPEHSLAEY | TCAGAAGTAGGACCTGAGCATAGTC | 358 | 393 |
| | NSP2 | | TTGCCGAATAC | | |
| | 376-387 | | | | |
| RS C6 1200 | cleavage | KMCG | AAAATGTGCGGA | 394 | 405 |
| | linker | | | | |
| RS C6 1200 | N 201 | YKHWPQIAQFAPSASAFFGMSRIGM | TACAAACATTGGCCGCAAATTGCAC | 406 | 606 |
| | | EVTPSGTWLTYTGAIKLDDKDPNFK | AATTTGCCCCCAGCGCTTCAGCGTT | | |
| | | DQVILLNKHIDAYKTFP | CTTCGGAATGTCGCGCATTGGCATG | | |
| | | | GAAGTCACACCTTCGGGAACGTGGT | | |
| | | | TGACCTACACAGGTGCCATCAAATT | | |
| | | | GGATGACAAAGATCCAAATTTCAAA | | |
| | | | GATCAAGTCATTTTGCTGAATAAGC | | |
| | | | ATATTGACGCATACAAAACATTCCC | | |
| | | | A | | |
| RS C6 1200 | cleavage | ARCA | GCTCGGTGTGCA | 607 | 618 |
| | linker | | | | |
| RS C6 1200 | ORF1ab | TTDPSFLGRYMSAL | ACAACTGATCCTAGTTTTCTGGGTA | 619 | 660 |
| | NSP3 | | GGTACATGTCAGCATTA | | |
| | 1635- | | | | |
| | 1650 | | | | |
| RS C6 1200 | ORF1ab | FADDLNQLTGY | TTTGCTGATGATTTAAACCAGTTAA | 661 | 693 |
| | NSP3 | | CTGGTTAT | | |
| | 1937- | | | | |
| | 1947 | | | | |
| RS C6 1200 | ORF1ab | HTDFSSEIIGY | CATACTGACTTTTCAAGTGAAATCA | 694 | 726 |
| | NSP4 | | TAGGATAC | | |
| | 2799- | | | | |
| | 2809 | | | | |
| RS C6 1200 | ORF1ab | QLMCQPILL | CAGCTTATGTGTCAACCTATACTGT | 727 | 753 |
| | NSP3 | | TA | | |
| | 2563- | | | | |
| | 2571 | | | | |
| RS C6 1200 | ORF1ab | AEAELAKNVSL | GCAGAAGCTGAACTTGCAAAGAATG | 754 | 786 |
| | NSP3 | | TGTCCTTA | | |
| | 2616- | | | | |
| | 2626 | | | | |
| RS C6 1200 | ORF1ab | ILGTVSWNL | ATTCTTGGAACTGTTTCTTGGAATT | 787 | 813 |
| | NSP3 | | TG | | |
| | 1367- | | | | |
| | 1375 | | | | |
| RS C6 1200 | cleavage | KKQG | AAAAAACAAGGT | 814 | 825 |
| | linker | | | | |
| RS C6 1200 | M 201 | FAYANRNRFLYIIKLIFLWLLWPVT | TTTGCCTATGCCAACAGGAATAGGT | 826 | 1026 |
| | | LACFVLAAVYRINWITGGIAIAMAC | TTTTGTATATAATTAAGTTAATTTT | | |
| | | LVGLMWLSYFIASFRLF | CCTCTGGCTGTTATGGCCAGTAACT | | |
| | | | TTAGCTTGTTTTGTGCTTGCTGCTG | | |
| | | | TTTACAGAATAAATTGGATCACCGG | | |
| | | | TGGAATTGCTATCGCAATGGCTTGT | | |
| | | | CTTGTAGGCTTGATGTGGCTCAGCT | | |
| | | | ACTTCATTGCTTCTTTCAGACTGTT | | |
| | | | T | | |
| RS C6 1200 | cleavage | YRSY | TACCGCTCCTAT | 1027 | 1038 |
| | linker | | | | |
| RS C6 1200 | ORF1ab | LLSAGIFGA | TTATTATCAGCTGGTATTTTTGGTG | 1039 | 1065 |
| | NSP3 | | CT | | |
| | 1148- | | | | |
| | 1156 | | | | |
| RS C6 1200 | ORF1ab | ITDVFYKENSY | ATTACGGATGTTTTCTACAAAGAAA | 1066 | 1098 |
| | NSP3 | | ACAGTTAC | | |
| | 1863- | | | | |
| | 1873 | | | | |
| RS C6 1200 | ORF1ab | KVPTDNYITTY | AAAGTGCCAACAGACAATTATATAA | 1099 | 1131 |
| | NSP3 | | CCACTTAC | | |
| | 1319- | | | | |
| | 1329 | | | | |
| RS C6 1200 | cleavage | ARMA | GCTCGGATGGCT | 1132 | 1143 |
| | linker | | | | |
| RS C6 1200 | ORF1ab | APKEIIFLEGETL | GCCCCAAAAGAAATTATCTTCTTAG | 1144 | 1182 |
| | NSP2 | | AGGGAGAAACACTT | | |
| | 735-747 | | | | |
| RS C6 1200 | ORF1ab | FGDDTVIEV | TTTGGTGATGACACTGTGATAGAAG | 1183 | 1209 |
| | NSP3 | | TG | | |
| | 825-833 | | | | |
| RS C6 1200 | cleavage | SMFN | TCTATGTTTAAC | 1210 | 1221 |
| | linker | | | | |
| RS C6 1200 | M 99 | LGRCDIKDLPKEITVATSRTLSYYK | CTAGGACGCTGTGACATCAAGGACC | 1222 | 1320 |
| | | LGASQRVA | TGCCTAAAGAAATCACTGTTGCTAC | | |
| | | | ATCACGAACGCTTTCTTATTACAAA | | |
| | | | TTGGGAGCTTCGCAGCGTGTAGCA | | |
| RS C6 1200 | end GSS | GGSLGGGGSG | GGTGGTTCTCTTGGCGGAGGGGGTT | 1321 | 1350 |
| | | | CGGGA | | |
| RS C6 1200 | MITD | IVGIVAGLAVLAVVVIGAVVATVMC | ATCGTGGGAATTGTGGCAGGACTGG | 1351 | 1521 |
| | | RRKSSGGKGGSYSQAASSDSAQGSD | CAGTGCTGGCCGTGGTGGTGATCGG | | |
| | | VSLTA** | AGCCGTGGTGGCTACCGTGATGTGC | | |
| | | | AGACGGAAGTCCAGCGGAGGCAAGG | | |
| | | | GCGGCAGCTACAGCCAGGCCGCCAG | | |
| | | | CTCTGATAGCGCCCAGGGCAGCGAC | | |
| | | | GTGTCACTGACAGCCTAGTAA | | |
| RS C7 1500 | SP1 | MRVMAPRTLILLLSGALALTETWAG | ATGAGAGTGATGGCCCCCAGAACCC | 1 | 78 |
| M chunks | | IS | TGATCCTGCTGCTGTCTGGCGCCCT | | |
| | | | GGCCCTGACAGAGACATGGGCCGGA | | |
| | | | AGC | | |
| RS C7 1500 | start GSS | GGSGGGGSGG | GGAGGTTCCGGAGGAGGCGGCAGTG | 79 | 108 |
| M chunks | | | GCGGC | | |
| RS C7 1500 | N 501 | SPARMAGNGGDAALALLLLDRLNQL | TCTCCTGCTAGAATGGCTGGCAATG | 109 | 609 |
| M chunks | | ESKMSGKGQQQQGQTVTKKSAAEAS | GCGGTGATGCTGCTCTTGCTTTGCT | | |
| | | KKPRQKRTATKAYNVTQAFGRRGPE | GCTGCTTGACAGATTGAACCAGCTT | | |
| | | QTQGNFGDQELIRQGTDYKHWPQIA | GAGAGCAAAATGTCTGGTAAAGGCC | | |
| | | QFAPSASAFFGMSRIGMEVTPSGTW | AACAACAACAAGGCCAAACTGTCAC | | |
| | | LTYTGAIKLDDKDPNFKDQVILLNK | TAAGAAATCTGCTGCTGAGGCTTCT | | |
| | | HIDAYKTFPPTEPKKDK | AAGAAGCCTCGGCAAAAACGTACTG | | |
| | | | CCACTAAAGCATACAATGTAACACA | | |
| | | | AGCTTTCGGCAGACGTGGTCCAGAA | | |
| | | | CAAACCCAAGGAAATTTTGGGGACC | | |
| | | | AGGAACTAATCAGACAAGGAACTGA | | |
| | | | TTACAAACATTGGCCGCAAATTGCA | | |
| | | | CAATTTGCCCCCAGCGCTTCAGCGT | | |
| | | | TCTTCGGAATGTCGCGCATTGGCAT | | |
| | | | GGAAGTCACACCTTCGGGAACGTGG | | |
| | | | TTGACCTACACAGGTGCCATCAAAT | | |
| | | | TGGATGACAAAGATCCAAATTTCAA | | |
| | | | AGATCAAGTCATTTTGCTGAATAAG | | |
| | | | CATATTGACGCATACAAAACATTCC | | |
| | | | CACCAACAGAGCCTAAAAAGGACAA | | |
| | | | A | | |
| RS C7 1500 | cleavage | FKAA | TTCAAAGCCGCA | 610 | 621 |
| M chunks | linker | | | | |
| RS C7 1500 | ORF1ab | MVTNNTFTLK | ATGGTAACAAACAATACCTTCACAC | 622 | 651 |
| M chunks | NSP2 | | TCAAA | | |
| | 807-816 | | | | |
| RS C7 1500 | NSP1 | VPHVGEIPVAYRKVLL | GTCCCTCATGTGGGCGAAATACCAG | 652 | 699 |
| M chunks | ORF1ab | | TGGCTTACCGCAAGGTTCTTCTT | | |
| | 108-123 | | | | |
| RS C7 1500 | ORF1ab | KTIQPRVEK | AAGACTATTCAACCAAGGGTTGAAA | 700 | 726 |
| M chunks | NSP2 | | AG | | |
| | 282-290 | | | | |
| RS C7 1500 | ORF1ab | YLFDESGEFKL | TACTTATTTGATGAGTCTGGTGAGT | 727 | 759 |
| M chunks | NSP3 | | TTAAATTG | | |
| | 906-916 | | | | |
| RS C7 1500 | ORF1ab | SEVGPEHSLAEY | TCAGAAGTAGGACCTGAGCATAGTC | 760 | 795 |
| M chunks | NSP2 | | TTGCCGAATAC | | |
| | 376-387 | | | | |
| RS C7 1500 | ORF1ab | YIFFASFYY | TACATCTTCTTTGCATCATTTTATT | 796 | 822 |
| M chunks | NSP3 | | AT | | |
| | 2384- | | | | |
| | 2392 | | | | |
| RS C7 1500 | cleavage | KRNG | AAGAGGAATGGT | 823 | 834 |
| M chunks | linker | | | | |
| RS C7 1500 | M 201 | FAYANRNRFLYIIKLIFLWLLWPVT | TTTGCCTATGCCAACAGGAATAGGT | 835 | 1035 |
| M chunks | | LACFVLAAVYRINWITGGIAIAMAC | TTTTGTATATAATTAAGTTAATTTT | | |
| | | LVGLMWLSYFIASFRLF | CCTCTGGCTGTTATGGCCAGTAACT | | |
| | | | TTAGCTTGTTTTGTGCTTGCTGCTG | | |
| | | | TTTACAGAATAAATTGGATCACCGG | | |
| | | | TGGAATTGCTATCGCAATGGCTTGT | | |
| | | | CTTGTAGGCTTGATGTGGCTCAGCT | | |
| | | | ACTTCATTGCTTCTTTCAGACTGTT | | |
| | | | T | | |
| RS C7 1500 | cleavage | YNSF | TACAATTCTTTC | 1036 | 1047 |
| M chunks | linker | | | | |
| RS C7 1500 | ORF1ab | HTTDPSFLGRYMSAL | CACACAACTGATCCTAGTTTTCTGG | 1048 | 1092 |
| M chunks | NSP3 | | GTAGGTACATGTCAGCATTA | | |
| | 1636- | | | | |
| | 1650 | | | | |
| RS C7 1500 | ORF1ab | FADDLNQLTGY | TTTGCTGATGATTTAAACCAGTTAA | 1093 | 1125 |
| M chunks | NSP3 | | CTGGTTAT | | |
| | 1937- | | | | |
| | 1947 | | | | |
| RS C7 1500 | ORF1ab | HTDFSSEIIGY | CATACTGACTTTTCAAGTGAAATCA | 1126 | 1158 |
| M chunks | NSP4 | | TAGGATAC | | |
| | 2799- | | | | |
| | 2809 | | | | |
| RS C7 1500 | ORF1ab | QLMCQPILL | CAGCTTATGTGTCAACCTATACTGT | 1159 | 1185 |
| M chunks | NSP3 | | TA | | |
| | 2563- | | | | |
| | 2571 | | | | |
| RS C7 1500 | ORF1ab | AEAELAKNVSL | GCAGAAGCTGAACTTGCAAAGAATG | 1186 | 1218 |
| M chunks | NSP3 | | TGTCCTTA | | |
| | 2616- | | | | |
| | 2626 | | | | |
| RS C7 1500 | ORF1ab | ILGTVSWNL | ATTCTTGGAACTGTTTCTTGGAATT | 1219 | 1245 |
| M chunks | NSP3 | | TG | | |
| | 1367- | | | | |
| | 1375 | | | | |
| RS C7 1500 | cleavage | KKNG | AAGAAAAACGGA | 1246 | 1257 |
| M chunks | linker | | | | |
| RS C7 1500 | M 99 | LGRCDIKDLPKEITVATSRTLSYYK | CTAGGACGCTGTGACATCAAGGACC | 1258 | 1356 |
| M chunks | | LGASQRVA | TGCCTAAAGAAATCACTGTTGCTAC | | |
| | | | ATCACGAACGCTTTCTTATTACAAA | | |
| | | | TTGGGAGCTTCGCAGCGTGTAGCA | | |
| RS C7 1500 | cleavage | YRSY | TACCGATCCTAC | 1357 | 1368 |
| M chunks | linker | | | | |
| RS C7 1500 | ORF1ab | LLSAGIFGA | TTATTATCAGCTGGTATTTTTGGTG | 1369 | 1395 |
| M chunks | NSP3 | | CT | | |
| | 1148- | | | | |
| | 1156 | | | | |
| RS C7 1500 | ORF1ab | ITDVFYKENSY | ATTACGGATGTTTTCTACAAAGAAA | 1396 | 1428 |
| M chunks | NSP3 | | ACAGTTAC | | |
| | 1863- | | | | |
| | 1873 | | | | |
| RS C7 1500 | ORF1ab | KVPTDNYITTY | AAAGTGCCAACAGACAATTATATAA | 1429 | 1461 |
| M chunks | NSP3 | | CCACTTAC | | |
| | 1319- | | | | |
| | 1329 | | | | |
| RS C7 1500 | cleavage | ARMA | GCTCGGATGGCG | 1462 | 1473 |
| M chunks | linker | | | | |
| RS C7 1500 | ORF1ab | APKEIIFLEGETL | GCCCCAAAAGAAATTATCTTCTTAG | 1474 | 1512 |
| M chunks | NSP2 | | AGGGAGAAACACTT | | |
| | 735-747 | | | | |
| RS C7 1500 | ORF1ab | FGDDTVIEV | TTTGGTGATGACACTGTGATAGAAG | 1513 | 1539 |
| M chunks | NSP3 | | TG | | |
| | 825-833 | | | | |
| RS C7 1500 | NSP2 | AIILASFSAST | GCCATTATTTTGGCATCTTTTTCTG | 1540 | 1572 |
| M chunks | ORF1ab | | CTTCCACA | | |
| | 474-484 | | | | |
| RS C7 1500 | cleavage | RRRG | AGGCGACGGGGT | 1573 | 1584 |
| M chunks | linker | | | | |
| RS C7 1500 | end GSS | GGSLGGGGSG | GGTGGTTCTCTTGGCGGAGGGGGTT | 1585 | 1614 |
| M chunks | | | CGGGA | | |
| RS C7 1500 | MITD | IVGIVAGLAVLAVVVIGAVVATVMC | ATCGTGGGAATTGTGGCAGGACTGG | 1615 | 1785 |
| M chunks | | RRKSSGGKGGSYSQAASSDSAQGSD | CAGTGCTGGCCGTGGTGGTGATCGG | | |
| | | VSLTA** | AGCCGTGGTGGCTACCGTGATGTGC | | |
| | | | AGACGGAAGTCCAGCGGAGGCAAGG | | |
| | | | GCGGCAGCTACAGCCAGGCCGCCAG | | |
| | | | CTCTGATAGCGCCCAGGGCAGCGAC | | |
| | | | GTGTCACTGACAGCCTAGTAA | | |
| RS C8 1500 | SP1 | MRVMAPRTLILLLSGALALTETWAG | ATGAGAGTGATGGCCCCCAGAACCC | 1 | 78 |
| M epitopes | | S | TGATCCTGCTGCTGTCTGGCGCCCT | | |
| | | | GGCCCTGACAGAGACATGGGCCGGA | | |
| | | | AGC | | |
| RS C8 1500 | start GSS | GGSGGGGSGG | GGAGGTTCCGGAGGAGGCGGCAGTG | 79 | 108 |
| M epitopes | | | GCGGC | | |
| RS C8 1500 | N 501 | SPARMAGNGGDAALALLLLDRLNQL | TCTCCTGCTAGAATGGCTGGCAATG | 109 | 609 |
| M epitopes | | ESKMSGKGQQQQGQTVTKKSAAEAS | GCGGTGATGCTGCTCTTGCTTTGCT | | |
| | | KKPRQKRTATKAYNVTQAFGRRGPE | GCTGCTTGACAGATTGAACCAGCTT | | |
| | | QTQGNFGDQELIRQGTDYKHWPQIA | GAGAGCAAAATGTCTGGTAAAGGCC | | |
| | | QFAPSASAFFGMSRIGMEVTPSGTW | AACAACAACAAGGCCAAACTGTCAC | | |
| | | LTYTGAIKLDDKDPNFKDQVILLNK | TAAGAAATCTGCTGCTGAGGCTTCT | | |
| | | HIDAYKTFPPTEPKKDK | AAGAAGCCTCGGCAAAAACGTACTG | | |
| | | | CCACTAAAGCATACAATGTAACACA | | |
| | | | AGCTTTCGGCAGACGTGGTCCAGAA | | |
| | | | CAAACCCAAGGAAATTTTGGGGACC | | |
| | | | AGGAACTAATCAGACAAGGAACTGA | | |
| | | | TTACAAACATTGGCCGCAAATTGCA | | |
| | | | CAATTTGCCCCCAGCGCTTCAGCGT | | |
| | | | TCTTCGGAATGTCGCGCATTGGCAT | | |
| | | | GGAAGTCACACCTTCGGGAACGTGG | | |
| | | | TTGACCTACACAGGTGCCATCAAAT | | |
| | | | TGGATGACAAAGATCCAAATTTCAA | | |
| | | | AGATCAAGTCATTTTGCTGAATAAG | | |
| | | | CATATTGACGCATACAAAACATTCC | | |
| | | | CACCAACAGAGCCTAAAAAGGACAA | | |
| | | | A | | |
| RS C8 1500 | cleavage | FRAC | TTTCGTGCGTGC | 610 | 621 |
| M epitopes | linker | | | | |
| RS C8 1500 | ORF1ab | MVTNNTFTLK | ATGGTAACAAACAATACCTTCACAC | 622 | 651 |
| M epitopes | NSP2 | | TCAAA | | |
| | 807-816 | | | | |
| RS C8 1500 | ORF1ab | VPHVGEIPVAYRKVLL | GTCCCTCATGTGGGCGAAATACCAG | 652 | 699 |
| M epitopes | NSP1 | | TGGCTTACCGCAAGGTTCTTCTT | | |
| | 108-123 | | | | |
| RS C8 1500 | ORF1ab | KTIQPRVEK | AAGACTATTCAACCAAGGGTTGAAA | 700 | 726 |
| M epitopes | NSP2 | | AG | | |
| | 282-290 | | | | |
| RS C8 1500 | ORF1ab | YLFDESGEFKL | TACTTATTTGATGAGTCTGGTGAGT | 727 | 759 |
| M epitopes | NSP3 | | TTAAATTG | | |
| | 906-916 | | | | |
| RS C8 1500 | ORF1ab | SEVGPEHSLAEY | TCAGAAGTAGGACCTGAGCATAGTC | 760 | 795 |
| M epitopes | NSP2 | | TTGCCGAATAC | | |
| | 376-387 | | | | |
| RS C8 1500 | ORF1ab | YIFFASFYY | TACATCTTCTTTGCATCATTTTATT | 796 | 822 |
| M epitopes | NSP3 | | AT | | |
| | 2384- | | | | |
| | 2392 | | | | |
| RS C8 1500 | cleavage | KRCF | AAGCGCTGTTTT | 823 | 834 |
| M epitopes | linker | | | | |
| RS C8 1500 | ORF1ab | HTTDPSFLGRYMSAL | CACACAACTGATCCTAGTTTTCTGG | 835 | 879 |
| M epitopes | NSP3 | | GTAGGTACATGTCAGCATTA | | |
| | 1636- | | | | |
| | 1650 | | | | |
| RS C8 1500 | ORF1ab | FADDLNQLTGY | TTTGCTGATGATTTAAACCAGTTAA | 880 | 912 |
| M epitopes | NSP3 | | CTGGTTAT | | |
| | 1937- | | | | |
| | 1947 | | | | |
| RS C8 1500 | ORF1ab | HTDFSSEIIGY | CATACTGACTTTTCAAGTGAAATCAT | 913 | 945 |
| M epitopes | NSP4 | | AGGATAC | | |
| | 2799- | | | | |
| | 2809 | | | | |
| RS C8 1500 | ORF1ab | QLMCQPILL | CAGCTTATGTGTCAACCTATACTGTTA | 946 | 972 |
| M epitopes | NSP3 | | | | |
| | 2563- | | | | |
| | 2571 | | | | |
| RS C8 1500 | ORF1ab | AEAELAKNVSL | GCAGAAGCTGAACTTGCAAAGAATGTG | 973 | 1005 |
| M epitopes | NSP3 | | TCCTTA | | |
| | 2616- | | | | |
| | 2626 | | | | |
| RS C8 1500 | ORF1ab | ILGTVSWNL | ATTCTTGGAACTGTTTCTTGGAATTTG | 1006 | 1032 |
| M epitopes | NSP3 | | | | |
| | 1367- | | | | |
| | 1375 | | | | |
| RS C8 1500 | cleavage | KKRY | AAAAAACGTTAC | 1033 | 1044 |
| M epitopes | linker | | | | |
| RS C8 1500 | ORF1ab | LLSAGIFGA | TTATTATCAGCTGGTATTTTTGGTGCT | 1045 | 1071 |
| M epitopes | NSP3 | | | | |
| | 1148- | | | | |
| | 1156 | | | | |
| RS C8 1500 | ORF1ab | ITDVFYKENSY | ATTACGGATGTTTTCTACAAAGAAAAC | 1072 | 1104 |
| M epitopes | NSP3 | | AGTTAC | | |
| | 1863- | | | | |
| | 1873 | | | | |
| RS C8 1500 | ORF1ab | KVPTDNYITTY | AAAGTGCCAACAGACAATTATATAACCA | 1105 | 1137 |
| M epitopes | NSP3 | | CTTAC | | |
| | 1319- | | | | |
| | 1329 | | | | |
| RS C8 1500 | cleavage | ARMA | GCGCGAATGGCA | 1138 | 1149 |
| M epitopes | linker | | | | |
| RS C8 1500 | ORF1ab | APKEIIFLEGETL | GCCCCAAAAGAAATTATCTTCTTAG | 1150 | 1188 |
| M epitopes | NSP2 | | AGGGAGAAACACTT | | |
| | 735-747 | | | | |
| RS C8 1500 | ORF1ab | FGDDTVIEV | TTTGGTGATGACACTGTGATAGAAG | 1189 | 1215 |
| M epitopes | NSP3 | | TG | | |
| | 825-833 | | | | |
| RS C8 1500 | ORF1ab | AIILASFSAST | GCCATTATTTTGGCATCTTTTTCTG | 1216 | 1248 |
| M epitopes | NSP2 | | CTTCCACA | | |
| | 474-484 | | | | |
| RS C8 1500 | cleavage | RRRG | CGCAGGCGCGGA | 1249 | 1260 |
| M epitopes | linker | | | | |
| RS C8 1500 | M 15-26 | KLLEQWNLVIGF | AAGCTCCTTGAACAATGGAACCTAG | 1261 | 1296 |
| M epitopes | | | TAATAGGTTTC | | |
| RS C8 1500 | M 41-71 | NRNRFLYIIKLIFLWLLWPVTLACF | AACAGGAATAGGTTTTTGTATATAA | 1297 | 1389 |
| M epitopes | | VLAAVY | TTAAGTTAATTTTCCTCTGGCTGTT | | |
| | | | ATGGCCAGTAACTTTAGCTTGTTTT | | |
| | | | GTGCTTGCTGCTGTTTAC | | |
| RS C8 1500 | M 136- | SELVIGAVILRGHLRIAGHHLGR | AGTGAACTCGTAATCGGAGCTGTGA | 1390 | 1458 |
| M epitopes | 158 | | TCCTTCGTGGACATCTTCGTATTGC | | |
| | | | TGGACACCATCTAGGACGC | | |
| RS C8 1500 | M 169- | TVATSRTLSYYKLGASQRV | ACTGTTGCTACATCACGAACGCTTT | 1459 | 1515 |
| M epitopes | 187 | | CTTATTACAAATTGGGAGCTTCGCA | | |
| | | | GCGTGTA | | |
| RS C8 1500 | cleavage | KRYS | AAACGCTACAGT | 1516 | 1527 |
| M epitopes | linker | | | | |
| RS C8 1500 | M 89-96 | GLMWLSYF | GGCTTGATGTGGCTCAGCTACTTC | 1528 | 1551 |
| M epitopes | | | | | |
| RS C8 1500 | cleavage | ARYA | GCGCGTTATGCC | 1552 | 1563 |
| M epitopes | linker | | | | |
| RS C8 1500 | end GSS | GGSLGGGGSG | GGTGGTTCTCTTGGCGGAGGGGGTT | 1564 | 1593 |
| M epitopes | | | CGGGA | | |
| RS C8 1500 | MITD | IVGIVAGLAVLAVVVIGAVVATVMC | ATCGTGGGAATTGTGGCAGGACTGG | 1594 | 1764 |
| M epitopes | | RRKSSGGKGGSYSQAASSDSAQGSD | CAGTGCTGGCCGTGGTGGTGATCGG | | |
| | | VSLTA** | AGCCGTGGTGGCTACCGTGATGTGC | | |
| | | | AGACGGAAGTCCAGCGGAGGCAAGG | | |
| | | | GCGGCAGCTACAGCCAGGCCGCCAG | | |
| | | | CTCTGATAGCGCCCAGGGCAGCGAC | | |
| | | | GTGTCACTGACAGCCTAGTAA | | |

TABLE 11
String sequences Group 1

| | | | AA | DNA |
|---|---|---|---|---|
| NAME | AA SEQUENCE | DNA SEQUENCE | LENGTH | LENGTH |
| RS C1- | NAMED SEGMENTS | | | |
| GSS | SP1-start GSS-nucleocapsid phosphoprotein-middle GSS-ORF9b protein-middle GSS- | | | |
| Linker | Orf1ab GSS-middle GSS-ORF3a protein-middle GSS-membrane glycoprotein-end GSS-MITD | | | |
| RS C1 | MRVMAPRTLILLLSGALALTETWAGSG | ATGAGAGTGATGGCCCCCAGAACCCTGATCCTGCTGCTG | 1369 | 4107 |
| GSS | GSGGGGSGGSDNGPQNQRNAPRITFGG | TCTGGCGCCCTGGCCCTGACAGAGACATGGGCCGGAAGC | | |
| linkers | PSDSTGSNQNGERSGARSKQRRPQGLP | GGCGGATCGGGGGGGGGGGGCTCGGGAGGATCTGATAA | | |
| | NNTASWFTALTQHGKEDLKFPRGQGVP | TGGACCCCAAAATCAGCGAAATGCACCCCGCATTACGTT | | |
| | INTNSSPDDQIGYYRRATRRIRGGDGKM | TGGTGGACCCTCAGATTCAACTGGCAGTAACCAGAATGG | | |
| | KDLSPRWYFYYLGTGPEAGLPYGANKD | AGAACGCAGTGGGGCGCGATCAAAACAACGTCGGCCCC | | |
| | GIIWVATEGALNTPKDHIGTRNPANNAA | AAGGTTTACCCAATAATACTGCGTCTTGGTTCACCGCTCT | | |
| | IVLQLPQGTTLPKGFYAEGSRGGSQASS | CACTCAACATGGCAAGGAAGACCTTAAATTCCCTCGAGG | | |
| | RSSSRSRNSSRNSTPGSSRGTSPARMAG | ACAAGGCGTTCCAATTAACACCAATAGCAGTCCAGATGA | | |
| | NGGDAALALLLLDRLNQLESKMSGKG | CCAAATTGGCTACTACCGAAGAGCTACCAGACGAATTCG | | |
| | QQQQGQTVTKKSAAEASKKPRQKRTAT | TGGTGGTGACGGTAAAATGAAAGATCTCAGTCCAAGATG | | |
| | KAYNVTQAFGRRGPEQTQGNFGDQELI | GTATTTCTACTACCTAGGAACTGGGCCAGAAGCTGGACT | | |
| | RQGTDYKHWPQIAQFAPSASAFFGMSRI | TCCCTATGGTGCTAACAAAGACGGCATCATATGGGTTGC | | |
| | GMEVTPSGTWLTYTGAIKLDDKDPNFK | AACTGAGGGAGCCTTGAATACACCAAAAGATCACATTG | | |
| | DQVILLNKHIDAYKTFPPTEPKKDKKKK | GCACCCGCAATCCTGCTAACAATGCTGCAATCGTGCTAC | | |
| | ADETQALPQRQKKQQTVTLLPAADLDD | AACTTCCTCAAGGAACAACATTGCCAAAAGGCTTCTACG | | |
| | FSKQLQQSMSSADSTQAGGSGGGGSGG | CAGAAGGGAGCAGAGGCGGCAGTCAAGCCTCTTCTCGTT | | |
| | DPKISEMHPALRLVDPQIQLAVTRMENA | CCTCATCACGTAGTCGCAACAGTTCAAGAAATTCAACTC | | |
| | VGRDQNNVGPKVYPIILRLGSPLSLNMA | CAGGCAGCAGTAGGGGAACTTCTCCTGCTAGAATGGCTG | | |
| | RKTLNSLEDKAFQLTPIAVQMTKLATTE | GCAATGGCGGTGATGCTGCTCTTGCTTTGCTGCTGCTTGA | | |
| | ELPDEFVVVTVKGGSGGGGSGGYHFFH | CAGATTGAACCAGCTTGAGAGCAAAATGTCTGGTAAAGG | | |
| | TTDPSFLGRYMSALFADDLNQLTGYHT | CCAACAACAACAAGGCCAAACTGTCACTAAGAAATCTG | | |
| | DFSSEIIGYQLMCQPILLAEAELAKNVSL | CTGCTGAGGCTTCTAAGAAGCCTCGGCAAAAACGTACTG | | |
| | ILGTVSWNLKRRYLLSAGIFGAITDVFY | CCACTAAAGCATACAATGTAACACAAGCTTTCGGCAGAC | | |
| | KENSYKVPTDNYITTYARMAAPKEIIFL | GTGGTCCAGAACAAACCCAAGGAAATTTTGGGGACCAG | | |
| | EGETLFGDDTVIEVAIILASFSASTRRMA | GAACTAATCAGACAAGGAACTGATTACAAACATTGGCC | | |
| | MVTNNTFTLKVPHVGEIPVAYRKVLLK | GCAAATTGCACAATTTGCCCCCAGCGCTTCAGCGTTCTTC | | |
| | TIQPRVEKYLFDESGEFKLSEVGPEHSLA | GGAATGTCGCGCATTGGCATGGAAGTCACACCTTCGGGA | | |
| | EYYIFFASFYYKRNGGGSGGGGSGGDL | ACGTGGTTGACCTACACAGGTGCCATCAAATTGGATGAC | | |
| | FMRIFTIGTVTLKQGEIKDATPSDFVRAT | AAAGATCCAAATTTCAAAGATCAAGTCATTTTGCTGAAT | | |
| | ATIPIQASLPFGWLIVGVALLAVFQSASK | AAGCATATTGACGCATACAAAACATTCCCACCAACAGAG | | |
| | UITLKKRWQLALSKGVHFVCNLLLLFVT | CCTAAAAAGGACAAAAAGAAGAAGGCTGATGAAACTCA | | |
| | VYSHLLLVAAGLEAPFLYLYALVYFLQ | AGCCTTACCGCAGAGACAGAAGAAACAGCAAACTGTGA | | |
| | SINFVRIIMRLWLCWKCRSKNPLLYDAN | CTCTTCTTCCTGCTGCAGATTTGGATGATTTCTCCAAACA | | |
| | YFLCWHTNCYDYCIPYNSVTSSIVITSG | ATTGCAACAATCCATGAGCAGTGCTGACTCAACTCAGGC | | |
| | DGTTSPISEHDYQIGGYTEKWESGVKDC | CGGGGGAAGTGGAGGAGGGGGATCTGGAGGGGACCCCA | | |
| | VVLHSYFTSDYYQLYSTQLSTDTGVEH | AAATCAGCGAAATGCACCCCGCATTACGTTTGGTGGACC | | |
| | VTFFIYNKIVDEPEEHVQIHTIDGSSGVV | CTCAGATTCAACTGGCAGTAACCAGAATGGAGAACGCA | | |
| | NPVMEPIYDEPTTTTSVPLGGSGGGGSG | GTGGGGCGCGATCAAAACAACGTCGGCCCCAAGGTTTAC | | |
| | GADSNGTITVEELKKLLEQWNLVIGFLF | CCAATAATACTGCGTCTTGGTTCACCGCTCTCACTCAACA | | |
| | LTWICLLQFAYANRNRFLYIIKLIFLWLL | TGGCAAGGAAGACCTTAAATTCCCTCGAGGACAAGGCGT | | |
| | WPVTLACFVLAAVYRINWITGGIAIAM | TCCAATTAACACCAATAGCAGTCCAGATGACCAAATTGG | | |
| | ACLVGLMWLSYFIASFRLFARTRSMWS | CTACTACCGAAGAGCTACCAGACGAATTCGTGGTGGTGA | | |
| | FNPETNILLNVPLHGTILTRPLLESELVIG | CGGTAAAAGGGGGAAGTGGAGGAGGGGGATCTGGAGGG | | |
| | AVILRGHLRIAGHHLGRCDIKDLPKEITV | TATCATTTCTTTCACACAACTGATCCTAGTTTTCTGGGTA | | |
| | ATSRTLSYYKLGASQRVAGDSGFAAYS | GGTACATGTCAGCATTATTTGCTGATGATTTAAACCAGTT | | |
| | RYRIGNYKLNTDHSSSSDNIALLVQGGS | AACTGGTTATCATACTGACTTTTCAAGTGAAATCATAGG | | |
| | LGGGGSGIVGIVAGLAVLAVVVIGAVV | ATACCAGCTTATGTGTCAACCTATACTGTTAGCAGAAGC | | |
| | ATVMCRRKSSGGKGGSYSQAASSDSAQ | TGAACTTGCAAAGAATGTGTCCTTAATTCTTGGAACTGTT | | |
| | GSDVSLTA** (SEQ ID NO: RS | TCTTGGAATTTGAAGCGCCGTTATTTATTATCAGCTGGTA | | |
| | C1p1full) | TTTTTGGTGCTATTACGGATGTTTTCTACAAAGAAAACAG | | |
| | | TTACAAAGTGCCAACAGACAATTATATAACCACTTACGC | | |
| | | CCGAATGGCGGCCCCAAAAGAAATTATCTTCTTAGAGGG | | |
| | | AGAAACACTTTTTGGTGATGACACTGTGATAGAAGTGGC | | |
| | | CATTATTTTGGCATCTTTTTCTGCTTCCACACGAAGAATG | | |
| | | GCCATGGTAACAAACAATACCTTCACACTCAAAGTCCCT | | |
| | | CATGTGGGCGAAATACCAGTGGCTTACCGCAAGGTTCTT | | |
| | | CTTAAGACTATTCAACCAAGGGTTGAAAAGTACTTATTT | | |
| | | GATGAGTCTGGTGAGTTTAAATTGTCAGAAGTAGGACCT | | |
| | | GAGCATAGTCTTGCCGAATACTACATCTTCTTTGCATCAT | | |
| | | TTTATTATAAACGTAATGGTGGGGGAAGTGGAGGAGGG | | |
| | | GGATCTGGAGGGGATTTGTTTATGAGAATCTTCACAATT | | |
| | | GGAACTGTAACTTTGAAGCAAGGTGAAATCAAGGATGCT | | |
| | | ACTCCTTCAGATTTTGTTCGCGCTACTGCAACGATACCGA | | |
| | | TACAAGCCTCACTCCCTTTCGGATGGCTTATTGTTGGCGT | | |
| | | TGCACTTCTTGCTGTTTTTCAGAGCGCTTCCAAAATCATA | | |
| | | ACCCTCAAAAAGAGATGGCAACTAGCACTCTCCAAGGGT | | |
| | | GTTCACTTTGTTTGCAACTTGCTGTTGTTGTTTGTAACAG | | |
| | | TTTACTCACACCTTTTGCTCGTTGCTGCTGGCCTTGAAGC | | |
| | | CCCTTTTCTCTATCTTTATGCTTTAGTCTACTTCTTGCAGA | | |
| | | GTATAAACTTTGTAAGAATAATAATGAGGCTTTGGCTTT | | |
| | | GCTGGAAATGCCGTTCCAAAAACCCATTACTTTATGATG | | |
| | | CCAACTATTTTCTTTGCTGGCATACTAATTGTTACGACTA | | |
| | | TTGTATACCTTACAATAGTGTAACTTCTTCAATTGTCATT | | |
| | | ACTTCAGGTGATGGCACAACAAGTCCTATTTCTGAACAT | | |
| | | GACTACCAGATTGGTGGTTATACTGAAAAATGGGAATCT | | |
| | | GGAGTAAAAGACTGTGTTGTATTACACAGTTACTTCACT | | |
| | | TCAGACTATTACCAGCTGTACTCAACTCAATTGAGTACA | | |
| | | GACACTGGTGTTGAACATGTTACCTTCTTCATCTACAATA | | |
| | | AAATTGTTGATGAGCCTGAAGAACATGTCCAAATTCACA | | |
| | | CAATCGACGGTTCATCCGGAGTTGTTAATCCAGTAATGG | | |
| | | AACCAATTTATGATGAACCGACGACGACTACTAGCGTGC | | |
| | | CTTTGGGGGGAAGTGGAGGAGGGGGATCTGGAGGGGCA | | |
| | | GATTCCAACGGTACTATTACCGTTGAAGAGCTTAAAAAG | | |
| | | CTCCTTGAACAATGGAACCTAGTAATAGGTTTCCTATTCC | | |
| | | TTACATGGATTTGTCTTCTACAATTTGCCTATGCCAACAG | | |
| | | GAATAGGTTTTTGTATATAATTAAGTTAATTTTCCTCTGG | | |
| | | CTGTTATGGCCAGTAACTTTAGCTTGTTTTGTGCTTGCTG | | |
| | | CTGTTTACAGAATAAATTGGATCACCGGTGGAATTGCTA | | |
| | | TCGCAATGGCTTGTCTTGTAGGCTTGATGTGGCTCAGCTA | | |
| | | CTTCATTGCTTCTTTCAGACTGTTTGCGCGTACGCGTTCC | | |
| | | ATGTGGTCATTCAATCCAGAAACTAACATTCTTCTCAAC | | |
| | | GTGCCACTCCATGGCACTATTCTGACCAGACCGCTTCTA | | |
| | | GAAAGTGAACTCGTAATCGGAGCTGTGATCCTTCGTGGA | | |
| | | CATCTTCGTATTGCTGGACACCATCTAGGACGCTGTGAC | | |
| | | ATCAAGGACCTGCCTAAAGAAATCACTGTTGCTACATCA | | |
| | | CGAACGCTTTCTTATTACAAATTGGGAGCTTCGCAGCGT | | |
| | | GTAGCAGGTGACTCAGGTTTTGCTGCATACAGTCGCTAC | | |
| | | AGGATTGGCAACTATAAATTAAACACAGACCATTCCAGT | | |
| | | AGCAGTGACAATATTGCTTTGCTTGTACAGGGAGGGAGC | | |
| | | CTGGGTGGGGGCGGCAGTGGGATCGTGGGAATTGTGGC | | |
| | | AGGACTGGCAGTGCTGGCCGTGGTGGTGATCGGAGCCGT | | |
| | | GGTGGCTACCGTGATGTGCAGACGGAAGTCCAGCGGAG | | |
| | | GCAAGGGCGGCAGCTACAGCCAGGCCGCCAGCTCTGAT | | |
| | | AGCGCCCAGGGCAGCGACGTGTCACTGACAGCCTAGTA | | |
| | | A (SEQ ID NO: RS C1n1) | | |
| RS C2 | | | | |
| GSS | NAMED SEGMENTS | | | |
| linkers | SP1-start GSS-membrane glycoprotein-middle GSS-ORF3a protein-middle GSS-Orf1ab GSS- | | | |
| inverted | middle GSS-ORF9b protein-middle GSS-nucleocapsid phosphoprotein-end GSS-MITD | | | |
| RS C2 | MRVMAPRTLILLLSGALALTETWAGSG | ATGAGAGTGATGGCCCCCAGAACCCTGATCCTGCTGCTG | 1369 | 4107 |
| GSS | GSGGGGSGGADSNGTITVEELKKLLEQ | TCTGGCGCCCTGGCCCTGACAGAGACATGGGCCGGAAGC | | |
| linkers- | WNLVIGFLFLTWICLLQFAYANRNRFLY | GGCGGATCGGGGGGGGGGGGCTCGGGAGGAGCAGATTC | | |
| inverted | IIKLIFLWLLWPVTLACFVLAAVYRINWI | CAACGGTACTATTACCGTTGAAGAGCTTAAAAAGCTCCT | | |
| | TGGIAIAMACLVGLMWLSYFIASFRLFA | TGAACAATGGAACCTAGTAATAGGTTTCCTATTCCTTAC | | |
| | RTRSMWSFNPETNILLNVPLHGTILTRPL | ATGGATTTGTCTTCTACAATTTGCCTATGCCAACAGGAAT | | |
| | LESELVIGAVILRGHLRIAGHHLGRCDIK | AGGTTTTTGTATATAATTAAGTTAATTTTCCTCTGGCTGT | | |
| | DLPKEITVATSRTLSYYKLGASQRVAGD | TATGGCCAGTAACTTTAGCTTGTTTTGTGCTTGCTGCTGT | | |
| | SGFAAYSRYRIGNYKLNTDHSSSSDNIA | TTACAGAATAAATTGGATCACCGGTGGAATTGCTATCGC | | |
| | LLVQGGSGGGGSGGDLFMRIFTIGTVTL | AATGGCTTGTCTTGTAGGCTTGATGTGGCTCAGCTACTTC | | |
| | KQGEIKDATPSDFVRATATIPIQASLPFG | ATTGCTTCTTTCAGACTGTTTGCGCGTACGCGTTCCATGT | | |
| | WLIVGVALLAVFQSASKIITLKKRWQLA | GGTCATTCAATCCAGAAACTAACATTCTTCTCAACGTGC | | |
| | LSKGVHFVCNLLLLFVTVYSHLLLVAA | CACTCCATGGCACTATTCTGACCAGACCGCTTCTAGAAA | | |
| | GLEAPFLYLYALVYFLQSINFVRIIMRL | GTGAACTCGTAATCGGAGCTGTGATCCTTCGTGGACATC | | |
| | WLCWKCRSKNPLLYDANYFLCWHTNC | TTCGTATTGCTGGACACCATCTAGGACGCTGTGACATCA | | |
| | YDYCIPYNSVTSSIVITSGDGTTSPISEHD | AGGACCTGCCTAAAGAAATCACTGTTGCTACATCACGAA | | |
| | YQIGGYTEKWESGVKDCVVLHSYFTSD | CGCTTTCTTATTACAAATTGGGAGCTTCGCAGCGTGTAGC | | |
| | YYQLYSTQLSTDTGVEHVTFFIYNKIVD | AGGTGACTCAGGTTTTGCTGCATACAGTCGCTACAGGAT | | |
| | EPEEHVQIHTIDGSSGVVNPVMEPIYDEP | TGGCAACTATAAATTAAACACAGACCATTCCAGTAGCAG | | |
| | TTTTSVPLGGSGGGGSGGYHFFHTTDPS | TGACAATATTGCTTTGCTTGTACAGGGGGGAAGTGGAGG | | |
| | FLGRYMSALFADDLNQLTGYHTDFSSEI | AGGGGGATCTGGAGGGGATTTGTTTATGAGAATCTTCAC | | |
| | IGYQLMCQPILLAEAELAKNVSLILGTV | AATTGGAACTGTAACTTTGAAGCAAGGTGAAATCAAGGA | | |
| | SWNLKRRYLLSAGIFGAITDVFYKENSY | TGCTACTCCTTCAGATTTTGTTCGCGCTACTGCAACGATA | | |
| | KVPTDNYITTYARMAAPKEIIFLEGETLF | CCGATACAAGCCTCACTCCCTTTCGGATGGCTTATTGTTG | | |
| | GDDTVIEVAIILASFSASTRRMAMVTNN | GCGTTGCACTTCTTGCTGTTTTTCAGAGCGCTTCCAAAAT | | |
| | TFTLKVPHVGEIPVAYRKVLLKTIQPRV | CATAACCCTCAAAAAGAGATGGCAACTAGCACTCTCCAA | | |
| | EKYLFDESGEFKLSEVGPEHSLAEYYIFF | GGGTGTTCACTTTGTTTGCAACTTGCTGTTGTTGTTTGTA | | |
| | ASFYYKRNGGGSGGGGSGGDPKISEMH | ACAGTTTACTCACACCTTTTGCTCGTTGCTGCTGGCCTTG | | |
| | PALRLVDPQIQLAVTRMENAVGRDQNN | AAGCCCCTTTTCTCTATCTTTATGCTTTAGTCTACTTCTTG | | |
| | VGPKVYPIILRLGSPLSLNMARKTLNSLE | CAGAGTATAAACTTTGTAAGAATAATAATGAGGCTTTGG | | |
| | DKAFQLTPIAVQMTKLATTEELPDEFVV | CTTTGCTGGAAATGCCGTTCCAAAAACCCATTACTTTATG | | |
| | VTVKGGSGGGGSGGSDNGPQNQRNAP | ATGCCAACTATTTTCTTTGCTGGCATACTAATTGTTACGA | | |
| | RITFGGPSDSTGSNQNGERSGARSKQRR | CTATTGTATACCTTACAATAGTGTAACTTCTTCAATTGTC | | |
| | PQGLPNNTASWFTALTQHGKEDLKFPR | ATTACTTCAGGTGATGGCACAACAAGTCCTATTTCTGAA | | |
| | GQGVPINTNSSPDDQIGYYRRATRRIRG | CATGACTACCAGATTGGTGGTTATACTGAAAAATGGGAA | | |
| | GDGKMKDLSPRWYFYYLGTGPEAGLP | TCTGGAGTAAAAGACTGTGTTGTATTACACAGTTACTTC | | |
| | YGANKDGIIWVATEGALNTPKDHIGTR | ACTTCAGACTATTACCAGCTGTACTCAACTCAATTGAGT | | |
| | NPANNAAIVLQLPQGTTLPKGFYAEGSR | ACAGACACTGGTGTTGAACATGTTACCTTCTTCATCTACA | | |
| | GGSQASSRSSSRSRNSSRNSTPGSSRGTS | ATAAAATTGTTGATGAGCCTGAAGAACATGTCCAAATTC | | |
| | PARMAGNGGDAALALLLLDRLNQLESK | ACACAATCGACGGTTCATCCGGAGTTGTTAATCCAGTAA | | |
| | MSGKGQQQQGQTVTKKSAAEASKKPR | TGGAACCAATTTATGATGAACCGACGACGACTACTAGCG | | |
| | QKRTATKAYNVTQAFGRRGPEQTQGNF | TGCCTTTGGGGGGAAGTGGAGGAGGGGGATCTGGAGGG | | |
| | GDQELIRQGTDYKHWPQIAQFAPSASAF | TATCATTTCTTTCACACAACTGATCCTAGTTTTCTGGGTA | | |
| | FGMSRIGMEVTPSGTWLTYTGAIKLDD | GGTACATGTCAGCATTATTTGCTGATGATTTAAACCAGTT | | |
| | KDPNFKDQVILLNKHIDAYKTFPPTEPK | AACTGGTTATCATACTGACTTTTCAAGTGAAATCATAGG | | |
| | KDKKKKADETQALPQRQKKQQTVTLLP | ATACCAGCTTATGTGTCAACCTATACTGTTAGCAGAAGC | | |
| | AADLDDESKQLQQSMSSADSTQAGGSL | TGAACTTGCAAAGAATGTGTCCTTAATTCTTGGAACTGTT | | |
| | GGGGSGIVGIVAGLAVLAVVVIGAVVA | TCTTGGAATTTGAAGCGCCGTTATTTATTATCAGCTGGTA | | |
| | TVMCRRKSSGGKGGSYSQAASSDSAQG | TTTTTGGTGCTATTACGGATGTTTTCTACAAAGAAAACAG | | |
| | SDVSLTA** (SEQ ID NO: RS | TTACAAAGTGCCAACAGACAATTATATAACCACTTACGC | | |
| | C2p1full) | CCGAATGGCGGCCCCAAAAGAAATTATCTTCTTAGAGGG | | |
| | | AGAAACACTTTTTGGTGATGACACTGTGATAGAAGTGGC | | |
| | | CATTATTTTGGCATCTTTTTCTGCTTCCACACGAAGAATG | | |
| | | GCCATGGTAACAAACAATACCTTCACACTCAAAGTCCCT | | |
| | | CATGTGGGCGAAATACCAGTGGCTTACCGCAAGGTTCTT | | |
| | | CTTAAGACTATTCAACCAAGGGTTGAAAAGTACTTATTT | | |
| | | GATGAGTCTGGTGAGTTTAAATTGTCAGAAGTAGGACCT | | |
| | | GAGCATAGTCTTGCCGAATACTACATCTTCTTTGCATCAT | | |
| | | TTTATTATAAACGTAATGGTGGGGGAAGTGGAGGAGGG | | |
| | | GGATCTGGAGGGGACCCCAAAATCAGCGAAATGCACCC | | |
| | | CGCATTACGTTTGGTGGACCCTCAGATTCAACTGGCAGT | | |
| | | AACCAGAATGGAGAACGCAGTGGGGCGCGATCAAAACA | | |
| | | ACGTCGGCCCCAAGGTTTACCCAATAATACTGCGTCTTG | | |
| | | GTTCACCGCTCTCACTCAACATGGCAAGGAAGACCTTAA | | |
| | | ATTCCCTCGAGGACAAGGCGTTCCAATTAACACCAATAG | | |
| | | CAGTCCAGATGACCAAATTGGCTACTACCGAAGAGCTAC | | |
| | | CAGACGAATTCGTGGTGGTGACGGTAAAAGGGGGAAGT | | |
| | | GGAGGAGGGGGATCTGGAGGGTCTGATAATGGACCCCA | | |
| | | AAATCAGCGAAATGCACCCCGCATTACGTTTGGTGGACC | | |
| | | CTCAGATTCAACTGGCAGTAACCAGAATGGAGAACGCA | | |
| | | GTGGGGCGCGATCAAAACAACGTCGGCCCCAAGGTTTAC | | |
| | | CCAATAATACTGCGTCTTGGTTCACCGCTCTCACTCAACA | | |
| | | TGGCAAGGAAGACCTTAAATTCCCTCGAGGACAAGGCGT | | |
| | | TCCAATTAACACCAATAGCAGTCCAGATGACCAAATTGG | | |
| | | CTACTACCGAAGAGCTACCAGACGAATTCGTGGTGGTGA | | |
| | | CGGTAAAATGAAAGATCTCAGTCCAAGATGGTATTTCTA | | |
| | | CTACCTAGGAACTGGGCCAGAAGCTGGACTTCCCTATGG | | |
| | | TGCTAACAAAGACGGCATCATATGGGTTGCAACTGAGGG | | |
| | | AGCCTTGAATACACCAAAAGATCACATTGGCACCCGCAA | | |
| | | TCCTGCTAACAATGCTGCAATCGTGCTACAACTTCCTCA | | |
| | | AGGAACAACATTGCCAAAAGGCTTCTACGCAGAAGGGA | | |
| | | GCAGAGGCGGCAGTCAAGCCTCTTCTCGTTCCTCATCAC | | |
| | | GTAGTCGCAACAGTTCAAGAAATTCAACTCCAGGCAGCA | | |
| | | GTAGGGGAACTTCTCCTGCTAGAATGGCTGGCAATGGCG | | |
| | | GTGATGCTGCTCTTGCTTTGCTGCTGCTTGACAGATTGAA | | |
| | | CCAGCTTGAGAGCAAAATGTCTGGTAAAGGCCAACAAC | | |
| | | AACAAGGCCAAACTGTCACTAAGAAATCTGCTGCTGAGG | | |
| | | CTTCTAAGAAGCCTCGGCAAAAACGTACTGCCACTAAAG | | |
| | | CATACAATGTAACACAAGCTTTCGGCAGACGTGGTCCAG | | |
| | | AACAAACCCAAGGAAATTTTGGGGACCAGGAACTAATC | | |
| | | AGACAAGGAACTGATTACAAACATTGGCCGCAAATTGC | | |
| | | ACAATTTGCCCCCAGCGCTTCAGCGTTCTTCGGAATGTC | | |
| | | GCGCATTGGCATGGAAGTCACACCTTCGGGAACGTGGTT | | |
| | | GACCTACACAGGTGCCATCAAATTGGATGACAAAGATCC | | |
| | | AAATTTCAAAGATCAAGTCATTTTGCTGAATAAGCATAT | | |
| | | TGACGCATACAAAACATTCCCACCAACAGAGCCTAAAA | | |
| | | AGGACAAAAAGAAGAAGGCTGATGAAACTCAAGCCTTA | | |
| | | CCGCAGAGACAGAAGAAACAGCAAACTGTGACTCTTCTT | | |
| | | CCTGCTGCAGATTTGGATGATTTCTCCAAACAATTGCAA | | |
| | | CAATCCATGAGCAGTGCTGACTCAACTCAGGCCGGAGGG | | |
| | | AGCCTGGGTGGGGGCGGCAGTGGGATCGTGGGAATTGT | | |
| | | GGCAGGACTGGCAGTGCTGGCCGTGGTGGTGATCGGAG | | |
| | | CCGTGGTGGCTACCGTGATGTGCAGACGGAAGTCCAGCG | | |
| | | GAGGCAAGGGCGGCAGCTACAGCCAGGCCGCCAGCTCT | | |
| | | GATAGCGCCCAGGGCAGCGACGTGTCACTGACAGCCTA | | |
| | | GTAA (SEQ ID NO: RS C2n1) | | |
| RS C3 | NAMED SEGMENTS | | | |
| 2A | SP1-start GSS-nucleocapsid phosphoprotein-1 GSG T2A-ORF9b protein-2 GSG P2A-Orf1ab | | | |
| linkers | GSS-3 GSG F2A-ORF3a protein-4 GSG E2A-membrane glycoprotein-end GSS-MITD | | | |
| RS C3 | MRVMAPRTLILLLSGALALTETWAGSG | ATGAGAGTGATGGCCCCCAGAACCCTGATCCTGCTGCTG | 1429 | 4287 |
| 2A | GSGGGGSGGSDNGPQNQRNAPRITFGG | TCTGGCGCCCTGGCCCTGACAGAGACATGGGCCGGAAGC | | |
| linkers | PSDSTGSNQNGERSGARSKQRRPQGLP | GGCGGATCGGGGGGGGGGGGCTCGGGAGGATCTGATAA | | |
| | NNTASWFTALTQHGKEDLKFPRGQGVP | TGGACCCCAAAATCAGCGAAATGCACCCCGCATTACGTT | | |
| | INTNSSPDDQIGYYRRATRRIRGGDGKM | TGGTGGACCCTCAGATTCAACTGGCAGTAACCAGAATGG | | |
| | KDLSPRWYFYYLGTGPEAGLPYGANKD | AGAACGCAGTGGGGCGCGATCAAAACAACGTCGGCCCC | | |
| | GIIWVATEGALNTPKDHIGTRNPANNAA | AAGGTTTACCCAATAATACTGCGTCTTGGTTCACCGCTCT | | |
| | IVLQLPQGTTLPKGFYAEGSRGGSQASS | CACTCAACATGGCAAGGAAGACCTTAAATTCCCTCGAGG | | |
| | RSSSRSRNSSRNSTPGSSRGTSPARMAG | ACAAGGCGTTCCAATTAACACCAATAGCAGTCCAGATGA | | |
| | NGGDAALALLLLDRLNQLESKMSGKG | CCAAATTGGCTACTACCGAAGAGCTACCAGACGAATTCG | | |
| | QQQQGQTVTKKSAAEASKKPRQKRTAT | TGGTGGTGACGGTAAAATGAAAGATCTCAGTCCAAGATG | | |
| | KAYNVTQAFGRRGPEQTQGNFGDQELI | GTATTTCTACTACCTAGGAACTGGGCCAGAAGCTGGACT | | |
| | RQGTDYKHWPQIAQFAPSASAFFGMSRI | TCCCTATGGTGCTAACAAAGACGGCATCATATGGGTTGC | | |
| | GMEVTPSGTWLTYTGAIKLDDKDPNFK | AACTGAGGGAGCCTTGAATACACCAAAAGATCACATTG | | |
| | DQVILLNKHIDAYKTFPPTEPKKDKKKK | GCACCCGCAATCCTGCTAACAATGCTGCAATCGTGCTAC | | |
| | ADETQALPQRQKKQQTVTLLPAADLDD | AACTTCCTCAAGGAACAACATTGCCAAAAGGCTTCTACG | | |
| | FSKQLQQSMSSADSTQAGSGGSGEGRG | CAGAAGGGAGCAGAGGCGGCAGTCAAGCCTCTTCTCGTT | | |
| | SLLTCGDVEENPGPDPKISEMHPALRLV | CCTCATCACGTAGTCGCAACAGTTCAAGAAATTCAACTC | | |
| | DPQIQLAVTRMENAVGRDQNNVGPKV | CAGGCAGCAGTAGGGGAACTTCTCCTGCTAGAATGGCTG | | |
| | YPIILRLGSPLSLNMARKTLNSLEDKAFQ | GCAATGGCGGTGATGCTGCTCTTGCTTTGCTGCTGCTTGA | | |
| | LTPIAVQMTKLATTEELPDEFVVVTVKG | CAGATTGAACCAGCTTGAGAGCAAAATGTCTGGTAAAGG | | |
| | SGGSGATNFSLLKQAGDVEENPGPYHF | CCAACAACAACAAGGCCAAACTGTCACTAAGAAATCTG | | |
| | FHTTDPSFLGRYMSALFADDLNQLTGY | CTGCTGAGGCTTCTAAGAAGCCTCGGCAAAAACGTACTG | | |
| | HTDFSSEIIGYQLMCQPILLAEAELAKNV | CCACTAAAGCATACAATGTAACACAAGCTTTCGGCAGAC | | |
| | SLILGTVSWNLKRRYLLSAGIFGAITDVF | GTGGTCCAGAACAAACCCAAGGAAATTTTGGGGACCAG | | |
| | YKENSYKVPTDNYITTYARMAAPKEIIF | GAACTAATCAGACAAGGAACTGATTACAAACATTGGCC | | |
| | LEGETLFGDDTVIEVAIILASFSASTRRM | GCAAATTGCACAATTTGCCCCCAGCGCTTCAGCGTTCTTC | | |
| | AMVTNNTFTLKVPHVGEIPVAYRKVLL | GGAATGTCGCGCATTGGCATGGAAGTCACACCTTCGGGA | | |
| | KTIQPRVEKYLFDESGEFKLSEVGPEHSL | ACGTGGTTGACCTACACAGGTGCCATCAAATTGGATGAC | | |
| | AEYYIFFASFYYKRNGGSGVKQTLNFD | AAAGATCCAAATTTCAAAGATCAAGTCATTTTGCTGAAT | | |
| | LLKLAGDVESNPGPDLFMRIFTIGTVTL | AAGCATATTGACGCATACAAAACATTCCCACCAACAGAG | | |
| | KQGEIKDATPSDFVRATATIPIQASLPFG | CCTAAAAAGGACAAAAAGAAGAAGGCTGATGAAACTCA | | |
| | WLIVGVALLAVFQSASKIITLKKRWQLA | AGCCTTACCGCAGAGACAGAAGAAACAGCAAACTGTGA | | |
| | LSKGVHFVCNLLLLFVTVYSHLLLVAA | CTCTTCTTCCTGCTGCAGATTTGGATGATTTCTCCAAACA | | |
| | GLEAPFLYLYALVYFLQSINFVRIIMRL | ATTGCAACAATCCATGAGCAGTGCTGACTCAACTCAGGC | | |
| | WLCWKCRSKNPLLYDANYFLCWHTNC | CGGCAGCGGCGGCTCCGGCGAGGGCAGGGGAAGTCTTC | | |
| | YDYCIPYNSVTSSIVITSGDGTTSPISEHD | TAACATGCGGGGACGTGGAGGAAAATCCCGGCCCAGAC | | |
| | YQIGGYTEKWESGVKDCVVLHSYFTSD | CCCAAAATCAGCGAAATGCACCCCGCATTACGTTTGGTG | | |
| | YYQLYSTQLSTDTGVEHVTFFIYNKIVD | GACCCTCAGATTCAACTGGCAGTAACCAGAATGGAGAA | | |
| | EPEEHVQIHTIDGSSGVVNPVMEPIYDEP | CGCAGTGGGGCGCGATCAAAACAACGTCGGCCCCAAGG | | |
| | TTTTSVPLGSGGSGQCTNYALLKLAGD | TTTACCCAATAATACTGCGTCTTGGTTCACCGCTCTCACT | | |
| | VESNPGPADSNGTITVEELKKLLEQWNL | CAACATGGCAAGGAAGACCTTAAATTCCCTCGAGGACA | | |
| | VIGFLFLTWICLLQFAYANRNRFLYIIKLI | AGGCGTTCCAATTAACACCAATAGCAGTCCAGATGACCA | | |
| | FLWLLWPVTLACFVLAAVYRINWITGGI | AATTGGCTACTACCGAAGAGCTACCAGACGAATTCGTGG | | |
| | AIAMACLVGLMWLSYFIASFRLFARTRS | TGGTGACGGTAAAAGGCAGCGGCGGCAGCGGCGCCACA | | |
| | MWSFNPETNILLNVPLHGTILTRPLLESE | AACTTCTCTCTGCTAAAGCAAGCAGGTGATGTTGAAGAA | | |
| | LVIGAVILRGHLRIAGHHLGRCDIKDLP | AACCCCGGGCCTTATCATTTCTTTCACACAACTGATCCTA | | |
| | KEITVATSRTLSYYKLGASQRVAGDSGF | GTTTTCTGGGTAGGTACATGTCAGCATTATTTGCTGATGA | | |
| | AAYSRYRIGNYKLNTDHSSSSDNIALLV | TTTAAACCAGTTAACTGGTTATCATACTGACTTTTCAAGT | | |
| | QGGSLGGGGSGIVGIVAGLAVLAVVVI | GAAATCATAGGATACCAGCTTATGTGTCAACCTATACTG | | |
| | GAVVATVMCRRKSSGGKGGSYSQAAS | TTAGCAGAAGCTGAACTTGCAAAGAATGTGTCCTTAATT | | |
| | SDSAQGSDVSLTA** (SEQ ID NO: RS | CTTGGAACTGTTTCTTGGAATTTGAAGCGCCGTTATTTAT | | |
| | C3p1full) | TATCAGCTGGTATTTTTGGTGCTATTACGGATGTTTTCTA | | |
| | | CAAAGAAAACAGTTACAAAGTGCCAACAGACAATTATA | | |
| | | TAACCACTTACGCCCGAATGGCGGCCCCAAAAGAAATTA | | |
| | | TCTTCTTAGAGGGAGAAACACTTTTTGGTGATGACACTG | | |
| | | TGATAGAAGTGGCCATTATTTTGGCATCTTTTTCTGCTTC | | |
| | | CACACGAAGAATGGCCATGGTAACAAACAATACCTTCAC | | |
| | | ACTCAAAGTCCCTCATGTGGGCGAAATACCAGTGGCTTA | | |
| | | CCGCAAGGTTCTTCTTAAGACTATTCAACCAAGGGTTGA | | |
| | | AAAGTACTTATTTGATGAGTCTGGTGAGTTTAAATTGTCA | | |
| | | GAAGTAGGACCTGAGCATAGTCTTGCCGAATACTACATC | | |
| | | TTCTTTGCATCATTTTATTATAAACGTAATGGTGGCAGCG | | |
| | | GCGTGAAACAGACTTTGAATTTTGACCTTCTCAAGTTGG | | |
| | | CGGGAGACGTGGAGTCCAACCCTGGACCTGATTTGTTTA | | |
| | | TGAGAATCTTCACAATTGGAACTGTAACTTTGAAGCAAG | | |
| | | GTGAAATCAAGGATGCTACTCCTTCAGATTTTGTTCGCGC | | |
| | | TACTGCAACGATACCGATACAAGCCTCACTCCCTTTCGG | | |
| | | ATGGCTTATTGTTGGCGTTGCACTTCTTGCTGTTTTTCAG | | |
| | | AGCGCTTCCAAAATCATAACCCTCAAAAAGAGATGGCA | | |
| | | ACTAGCACTCTCCAAGGGTGTTCACTTTGTTTGCAACTTG | | |
| | | CTGTTGTTGTTTGTAACAGTTTACTCACACCTTTTGCTCG | | |
| | | TTGCTGCTGGCCTTGAAGCCCCTTTTCTCTATCTTTATGCT | | |
| | | TTAGTCTACTTCTTGCAGAGTATAAACTTTGTAAGAATAA | | |
| | | TAATGAGGCTTTGGCTTTGCTGGAAATGCCGTTCCAAAA | | |
| | | ACCCATTACTTTATGATGCCAACTATTTTCTTTGCTGGCA | | |
| | | TACTAATTGTTACGACTATTGTATACCTTACAATAGTGTA | | |
| | | ACTTCTTCAATTGTCATTACTTCAGGTGATGGCACAACAA | | |
| | | GTCCTATTTCTGAACATGACTACCAGATTGGTGGTTATAC | | |
| | | TGAAAAATGGGAATCTGGAGTAAAAGACTGTGTTGTATT | | |
| | | ACACAGTTACTTCACTTCAGACTATTACCAGCTGTACTCA | | |
| | | ACTCAATTGAGTACAGACACTGGTGTTGAACATGTTACC | | |
| | | TTCTTCATCTACAATAAAATTGTTGATGAGCCTGAAGAA | | |
| | | CATGTCCAAATTCACACAATCGACGGTTCATCCGGAGTT | | |
| | | GTTAATCCAGTAATGGAACCAATTTATGATGAACCGACG | | |
| | | ACGACTACTAGCGTGCCTTTGGGCAGCGGCGGCTCGGGC | | |
| | | CAGTGTACTAATTATGCTCTCTTGAAATTGGCTGGAGAT | | |
| | | GTTGAGAGCAACCCAGGTCCCGCAGATTCCAACGGTACT | | |
| | | ATTACCGTTGAAGAGCTTAAAAAGCTCCTTGAACAATGG | | |
| | | AACCTAGTAATAGGTTTCCTATTCCTTACATGGATTTGTC | | |
| | | TTCTACAATTTGCCTATGCCAACAGGAATAGGTTTTTGTA | | |
| | | TATAATTAAGTTAATTTTCCTCTGGCTGTTATGGCCAGTA | | |
| | | ACTTTAGCTTGTTTTGTGCTTGCTGCTGTTTACAGAATAA | | |
| | | ATTGGATCACCGGTGGAATTGCTATCGCAATGGCTTGTC | | |
| | | TTGTAGGCTTGATGTGGCTCAGCTACTTCATTGCTTCTTT | | |
| | | CAGACTGTTTGCGCGTACGCGTTCCATGTGGTCATTCAAT | | |
| | | CCAGAAACTAACATTCTTCTCAACGTGCCACTCCATGGC | | |
| | | ACTATTCTGACCAGACCGCTTCTAGAAAGTGAACTCGTA | | |
| | | ATCGGAGCTGTGATCCTTCGTGGACATCTTCGTATTGCTG | | |
| | | GACACCATCTAGGACGCTGTGACATCAAGGACCTGCCTA | | |
| | | AAGAAATCACTGTTGCTACATCACGAACGCTTTCTTATTA | | |
| | | CAAATTGGGAGCTTCGCAGCGTGTAGCAGGTGACTCAGG | | |
| | | TTTTGCTGCATACAGTCGCTACAGGATTGGCAACTATAA | | |
| | | ATTAAACACAGACCATTCCAGTAGCAGTGACAATATTGC | | |
| | | TTTGCTTGTACAGGGAGGGAGCCTGGGTGGGGGCGGCAG | | |
| | | TGGGATCGTGGGAATTGTGGCAGGACTGGCAGTGCTGGC | | |
| | | CGTGGTGGTGATCGGAGCCGTGGTGGCTACCGTGATGTG | | |
| | | CAGACGGAAGTCCAGCGGAGGCAAGGGCGGCAGCTACA | | |
| | | GCCAGGCCGCCAGCTCTGATAGCGCCCAGGGCAGCGAC | | |
| | | GTGTCACTGACAGCCTAGTAA (SEQ ID NO: RS C3n1) | | |
| RS C4 | | | | |
| ORF1ab | NAMED SEGMENTS | | | |
| as | SP1-start GSS-nucleocapsid phosphoprotein-Orf1ab linker 1-ORF9b protein-Orf1ab | | | |
| linkers | linker 2-ORF3a protein-Orf1ab linker 3-membrane glycoprotein-end GSS-MITD | | | |
| ORF1ab | MRVMAPRTLILLLSGALALTETWAGSG | ATGAGAGTGATGGCCCCCAGAACCCTGATCCTGCTGCTG | 1337 | 4011 |
| as | GSGGGGSGGSDNGPQNQRNAPRITFGG | TCTGGCGCCCTGGCCCTGACAGAGACATGGGCCGGAAGC | | |
| linkers | PSDSTGSNQNGERSGARSKQRRPQGLP | GGAGGGAGTGGTGGCGGGGGATCTGGAGGTTCTGATAA | | |
| | NNTASWFTALTQHGKEDLKFPRGQGVP | TGGACCCCAAAATCAGCGAAATGCACCCCGCATTACGTT | | |
| | INTNSSPDDQIGYYRRATRRIRGGDGKM | TGGTGGACCCTCAGATTCAACTGGCAGTAACCAGAATGG | | |
| | KDLSPRWYFYYLGTGPEAGLPYGANKD | AGAACGCAGTGGGGCGCGATCAAAACAACGTCGGCCCC | | |
| | GIIWVATEGALNTPKDHIGTRNPANNAA | AAGGTTTACCCAATAATACTGCGTCTTGGTTCACCGCTCT | | |
| | IVLQLPQGTTLPKGFYAEGSRGGSQASS | CACTCAACATGGCAAGGAAGACCTTAAATTCCCTCGAGG | | |
| | RSSSRSRNSSRNSTPGSSRGTSPARMAG | ACAAGGCGTTCCAATTAACACCAATAGCAGTCCAGATGA | | |
| | NGGDAALALLLLDRLNQLESKMSGKG | CCAAATTGGCTACTACCGAAGAGCTACCAGACGAATTCG | | |
| | QQQQGQTVTKKSAAEASKKPRQKRTAT | TGGTGGTGACGGTAAAATGAAAGATCTCAGTCCAAGATG | | |
| | KAYNVTQAFGRRGPEQTQGNFGDQELI | GTATTTCTACTACCTAGGAACTGGGCCAGAAGCTGGACT | | |
| | RQGTDYKHWPQIAQFAPSASAFFGMSRI | TCCCTATGGTGCTAACAAAGACGGCATCATATGGGTTGC | | |
| | GMEVTPSGTWLTYTGAIKLDDKDPNFK | AACTGAGGGAGCCTTGAATACACCAAAAGATCACATTG | | |
| | DQVILLNKHIDAYKTFPPTEPKKDKKKK | GCACCCGCAATCCTGCTAACAATGCTGCAATCGTGCTAC | | |
| | ADETQALPQRQKKQQTVTLLPAADLDD | AACTTCCTCAAGGAACAACATTGCCAAAAGGCTTCTACG | | |
| | FSKQLQQSMSSADSTQAFRACMVTNNT | CAGAAGGGAGCAGAGGCGGCAGTCAAGCCTCTTCTCGTT | | |
| | FTLKVPHVGEIPVAYRKVLLKTIQPRVE | CCTCATCACGTAGTCGCAACAGTTCAAGAAATTCAACTC | | |
| | KYLFDESGEFKLSEVGPEHSLAEYYIFF | CAGGCAGCAGTAGGGGAACTTCTCCTGCTAGAATGGCTG | | |
| | ASFYYKRNGDPKISEMHPALRLVDPQIQ | GCAATGGCGGTGATGCTGCTCTTGCTTTGCTGCTGCTTGA | | |
| | LAVTRMENAVGRDQNNVGPKVYPIILR | CAGATTGAACCAGCTTGAGAGCAAAATGTCTGGTAAAGG | | |
| | LGSPLSLNMARKTLNSLEDKAFQLTPIA | CCAACAACAACAAGGCCAAACTGTCACTAAGAAATCTG | | |
| | VQMTKLATTEELPDEFVVVTVKFNSFH | CTGCTGAGGCTTCTAAGAAGCCTCGGCAAAAACGTACTG | | |
| | TTDPSFLGRYMSALFADDLNQLTGYHT | CCACTAAAGCATACAATGTAACACAAGCTTTCGGCAGAC | | |
| | DFSSEIIGYQLMCQPILLAEAELAKNVSL | GTGGTCCAGAACAAACCCAAGGAAATTTTGGGGACCAG | | |
| | ILGTVSWNLKKQGDLFMRIFTIGTVTLK | GAACTAATCAGACAAGGAACTGATTACAAACATTGGCC | | |
| | QGEIKDATPSDFVRATATIPIQASLPFGW | GCAAATTGCACAATTTGCCCCCAGCGCTTCAGCGTTCTTC | | |
| | LIVGVALLAVFQSASKIITLKKRWQLAL | GGAATGTCGCGCATTGGCATGGAAGTCACACCTTCGGGA | | |
| | SKGVHFVCNLLLLFVTVYSHLLLVAAG | ACGTGGTTGACCTACACAGGTGCCATCAAATTGGATGAC | | |
| | LEAPFLYLYALVYFLQSINFVRIIMRLWL | AAAGATCCAAATTTCAAAGATCAAGTCATTTTGCTGAAT | | |
| | CWKCRSKNPLLYDANYFLCWHTNCYD | AAGCATATTGACGCATACAAAACATTCCCACCAACAGAG | | |
| | YCIPYNSVTSSIVITSGDGTTSPISEHDYQ | CCTAAAAAGGACAAAAAGAAGAAGGCTGATGAAACTCA | | |
| | IGGYTEKWESGVKDCVVLHSYFTSDYY | AGCCTTACCGCAGAGACAGAAGAAACAGCAAACTGTGA | | |
| | QLYSTQLSTDTGVEHVTFFIYNKIVDEPE | CTCTTCTTCCTGCTGCAGATTTGGATGATTTCTCCAAACA | | |
| | EHVQIHTIDGSSGVVNPVMEPIYDEPTTT | ATTGCAACAATCCATGAGCAGTGCTGACTCAACTCAGGC | | |
| | TSVPLRRSYLLSAGIFGAITDVFYKENSY | CTTTCGGGCCTGCATGGTAACAAACAATACCTTCACACT | | |
| | KVPTDNYITTYARMAAPKEIIFLEGETLF | CAAAGTCCCTCATGTGGGCGAAATACCAGTGGCTTACCG | | |
| | GDDTVIEVAIILASFSASTLLVQADSNGT | CAAGGTTCTTCTTAAGACTATTCAACCAAGGGTTGAAAA | | |
| | ITVEELKKLLEQWNLVIGFLFLTWICLL | GTACTTATTTGATGAGTCTGGTGAGTTTAAATTGTCAGAA | | |
| | QFAYANRNRFLYIIKLIFLWLLWPVTLA | GTAGGACCTGAGCATAGTCTTGCCGAATACTACATCTTC | | |
| | CFVLAAVYRINWITGGIAIAMACLVGL | TTTGCATCATTTTATTATAAGCGGAACGGCGACCCCAAA | | |
| | MWLSYFIASFRLFARTRSMWSFNPETNI | ATCAGCGAAATGCACCCCGCATTACGTTTGGTGGACCCT | | |
| | LLNVPLHGTILTRPLLESELVIGAVILRG | CAGATTCAACTGGCAGTAACCAGAATGGAGAACGCAGT | | |
| | HLRIAGHHLGRCDIKDLPKEITVATSRTL | GGGGCGCGATCAAAACAACGTCGGCCCCAAGGTTTACCC | | |
| | SYYKLGASQRVAGDSGFAAYSRYRIGN | AATAATACTGCGTCTTGGTTCACCGCTCTCACTCAACATG | | |
| | YKLNTDHSSSSDNIALLVQGGSLGGGGS | GCAAGGAAGACCTTAAATTCCCTCGAGGACAAGGCGTTC | | |
| | GIVGIVAGLAVLAVVVIGAVVATVMCR | CAATTAACACCAATAGCAGTCCAGATGACCAAATTGGCT | | |
| | RKSSGGKGGSYSQAASSDSAQGSDVSL | ACTACCGAAGAGCTACCAGACGAATTCGTGGTGGTGACG | | |
| | TA** (SEQ ID NO: RS C4p1full) | GTAAAATTTAACTCTTTTCACACAACTGATCCTAGTTTTC | | |
| | | TGGGTAGGTACATGTCAGCATTATTTGCTGATGATTTAA | | |
| | | ACCAGTTAACTGGTTATCATACTGACTTTTCAAGTGAAAT | | |
| | | CATAGGATACCAGCTTATGTGTCAACCTATACTGTTAGC | | |
| | | AGAAGCTGAACTTGCAAAGAATGTGTCCTTAATTCTTGG | | |
| | | AACTGTTTCTTGGAATTTGAAAAAGCAAGGAGATTTGTT | | |
| | | TATGAGAATCTTCACAATTGGAACTGTAACTTTGAAGCA | | |
| | | AGGTGAAATCAAGGATGCTACTCCTTCAGATTTTGTTCG | | |
| | | CGCTACTGCAACGATACCGATACAAGCCTCACTCCCTTT | | |
| | | CGGATGGCTTATTGTTGGCGTTGCACTTCTTGCTGTTTTT | | |
| | | CAGAGCGCTTCCAAAATCATAACCCTCAAAAAGAGATG | | |
| | | GCAACTAGCACTCTCCAAGGGTGTTCACTTTGTTTGCAA | | |
| | | CTTGCTGTTGTTGTTTGTAACAGTTTACTCACACCTTTTG | | |
| | | CTCGTTGCTGCTGGCCTTGAAGCCCCTTTTCTCTATCTTT | | |
| | | ATGCTTTAGTCTACTTCTTGCAGAGTATAAACTTTGTAAG | | |
| | | AATAATAATGAGGCTTTGGCTTTGCTGGAAATGCCGTTC | | |
| | | CAAAAACCCATTACTTTATGATGCCAACTATTTTCTTTGC | | |
| | | TGGCATACTAATTGTTACGACTATTGTATACCTTACAATA | | |
| | | GTGTAACTTCTTCAATTGTCATTACTTCAGGTGATGGCAC | | |
| | | AACAAGTCCTATTTCTGAACATGACTACCAGATTGGTGG | | |
| | | TTATACTGAAAAATGGGAATCTGGAGTAAAAGACTGTGT | | |
| | | TGTATTACACAGTTACTTCACTTCAGACTATTACCAGCTG | | |
| | | TACTCAACTCAATTGAGTACAGACACTGGTGTTGAACAT | | |
| | | GTTACCTTCTTCATCTACAATAAAATTGTTGATGAGCCTG | | |
| | | AAGAACATGTCCAAATTCACACAATCGACGGTTCATCCG | | |
| | | GAGTTGTTAATCCAGTAATGGAACCAATTTATGATGAAC | | |
| | | CGACGACGACTACTAGCGTGCCTTTGAGACGTTCTTATTT | | |
| | | ATTATCAGCTGGTATTTTTGGTGCTATTACGGATGTTTTC | | |
| | | TACAAAGAAAACAGTTACAAAGTGCCAACAGACAATTA | | |
| | | TATAACCACTTACGCTCGAATGGCTGCCCCAAAAGAAAT | | |
| | | TATCTTCTTAGAGGGAGAAACACTTTTTGGTGATGACAC | | |
| | | TGTGATAGAAGTGGCCATTATTTTGGCATCTTTTTCTGCT | | |
| | | TCCACACTACTTGTCCAGGCAGATTCCAACGGTACTATT | | |
| | | ACCGTTGAAGAGCTTAAAAAGCTCCTTGAACAATGGAAC | | |
| | | CTAGTAATAGGTTTCCTATTCCTTACATGGATTTGTCTTC | | |
| | | TACAATTTGCCTATGCCAACAGGAATAGGTTTTTGTATAT | | |
| | | AATTAAGTTAATTTTCCTCTGGCTGTTATGGCCAGTAACT | | |
| | | TTAGCTTGTTTTGTGCTTGCTGCTGTTTACAGAATAAATT | | |
| | | GGATCACCGGTGGAATTGCTATCGCAATGGCTTGTCTTG | | |
| | | TAGGCTTGATGTGGCTCAGCTACTTCATTGCTTCTTTCAG | | |
| | | ACTGTTTGCGCGTACGCGTTCCATGTGGTCATTCAATCCA | | |
| | | GAAACTAACATTCTTCTCAACGTGCCACTCCATGGCACT | | |
| | | ATTCTGACCAGACCGCTTCTAGAAAGTGAACTCGTAATC | | |
| | | GGAGCTGTGATCCTTCGTGGACATCTTCGTATTGCTGGAC | | |
| | | ACCATCTAGGACGCTGTGACATCAAGGACCTGCCTAAAG | | |
| | | AAATCACTGTTGCTACATCACGAACGCTTTCTTATTACAA | | |
| | | ATTGGGAGCTTCGCAGCGTGTAGCAGGTGACTCAGGTTT | | |
| | | TGCTGCATACAGTCGCTACAGGATTGGCAACTATAAATT | | |
| | | AAACACAGACCATTCCAGTAGCAGTGACAATATTGCTTT | | |
| | | GCTTGTACAGGGTGGCTCGTTAGGTGGCGGAGGGTCAGG | | |
| | | CATCGTGGGAATTGTGGCAGGACTGGCAGTGCTGGCCGT | | |
| | | GGTGGTGATCGGAGCCGTGGTGGCTACCGTGATGTGCAG | | |
| | | ACGGAAGTCCAGCGGAGGCAAGGGCGGCAGCTACAGCC | | |
| | | AGGCCGCCAGCTCTGATAGCGCCCAGGGCAGCGACGTGT | | |
| | | CACTGACAGCCTAGTAA (SEQ ID NO: RS C4n1) | | |

TABLE 12
String Sequences Group 2

| | | | AA | DNA |
|---|---|---|---|---|
| NAME | AA SEQUENCE | DNA SEQUENCE | LENGTH | LENGTH |
| | Named Segments: SP1-start GSS-N without Orf9b-Orf1ab 2300-membrane glycoprotein-end | | | |
| RS C5 | GSS-MITD | | | |
| RS C5 | MRVMAPRTLILLLSGALALTETWAGSG | ATGAGAGTGATGGCCCCCAGAACCCTGATCCTGCTGCT | 859 | 2577 |
| 2300 | GSGGGGSGGKDLSPRWYFYYLGTGPEA | GTCTGGCGCCCTGGCCCTGACAGAGACATGGGCCGGAA | | |
| | GLPYGANKDGIIWVATEGALNTPKDHI | GCGGAGGTTCCGGAGGAGGCGGCAGTGGCGGCAAAGA | | |
| | GTRNPANNAAIVLQLPQGTTLPKGFYA | TCTCAGTCCAAGATGGTATTTCTACTACCTAGGAACTGG | | |
| | EGSRGGSQASSRSSSRSRNSSRNSTPGSS | GCCAGAAGCTGGACTTCCCTATGGTGCTAACAAAGACG | | |
| | RGTSPARMAGNGGDAALALLLLDRLN | GCATCATATGGGTTGCAACTGAGGGAGCCTTGAATACA | | |
| | QLESKMSGKGQQQQGQTVTKKSAAEA | CCAAAAGATCACATTGGCACCCGCAATCCTGCTAACAA | | |
| | SKKPRQKRTATKAYNVTQAFGRRGPEQ | TGCTGCAATCGTGCTACAACTTCCTCAAGGAACAACATT | | |
| | TQGNFGDQELIRQGTDYKHWPQIAQFA | GCCAAAAGGCTTCTACGCAGAAGGGAGCAGAGGCGGC | | |
| | PSASAFFGMSRIGMEVTPSGTWLTYTG | AGTCAAGCCTCTTCTCGTTCCTCATCACGTAGTCGCAAC | | |
| | AIKLDDKDPNFKDQVILLNKHIDAYKTF | AGTTCAAGAAATTCAACTCCAGGCAGCAGTAGGGGAAC | | |
| | PPTEPKKDKKKKADETQALPQRQKKQQ | TTCTCCTGCTAGAATGGCTGGCAATGGCGGTGATGCTGC | | |
| | TVTLLPAADLDDFSKQLQQSMSSADST | TCTTGCTTTGCTGCTGCTTGACAGATTGAACCAGCTTGA | | |
| | QAFRACMVTNNTFTLKVPHVGEIPVAY | GAGCAAAATGTCTGGTAAAGGCCAACAACAACAAGGCC | | |
| | RKVLLKTIQPRVEKYLFDESGEFKLSEV | AAACTGTCACTAAGAAATCTGCTGCTGAGGCTTCTAAG | | |
| | GPEHSLAEYYIFFASFYYKRCFHTTDPSF | AAGCCTCGGCAAAAACGTACTGCCACTAAAGCATACAA | | |
| | LGRYMSALFADDLNQLTGYHTDESSEII | TGTAACACAAGCTTTCGGCAGACGTGGTCCAGAACAAA | | |
| | GYQLMCQPILLAEAELAKNVSLILGTVS | CCCAAGGAAATTTTGGGGACCAGGAACTAATCAGACAA | | |
| | WNLKKRYLLSAGIFGAITDVFYKENSY | GGAACTGATTACAAACATTGGCCGCAAATTGCACAATT | | |
| | KVPTDNYITTYARMAAPKEIIFLEGETLF | TGCCCCCAGCGCTTCAGCGTTCTTCGGAATGTCGCGCAT | | |
| | GDDTVIEVAIILASFSASTRRSGADSNGT | TGGCATGGAAGTCACACCTTCGGGAACGTGGTTGACCT | | |
| | ITVEELKKLLEQWNLVIGFLFLTWICLL | ACACAGGTGCCATCAAATTGGATGACAAAGATCCAAAT | | |
| | QFAYANRNRFLYIIKLIFLWLLWPVTLA | TTCAAAGATCAAGTCATTTTGCTGAATAAGCATATTGAC | | |
| | CFVLAAVYRINWITGGIAIAMACLVGL | GCATACAAAACATTCCCACCAACAGAGCCTAAAAAGGA | | |
| | MWLSYFIASFRLFARTRSMWSFNPETNI | CAAAAAGAAGAAGGCTGATGAAACTCAAGCCTTACCGC | | |
| | LLNVPLHGTILTRPLLESELVIGAVILRG | AGAGACAGAAGAAACAGCAAACTGTGACTCTTCTTCCT | | |
| | HLRIAGHHLGRCDIKDLPKEITVATSRT | GCTGCAGATTTGGATGATTTCTCCAAACAATTGCAACAA | | |
| | LSYYKLGASQRVAGDSGFAAYSRYRIG | TCCATGAGCAGTGCTGACTCAACTCAGGCCTTCCGCGCG | | |
| | NYKLNTDHSSSSDNIALLVQGGSLGGG | TGCATGGTAACAAACAATACCTTCACACTCAAAGTCCCT | | |
| | GSGIVGIVAGLAVLAVVVIGAVVATVM | CATGTGGGCGAAATACCAGTGGCTTACCGCAAGGTTCT | | |
| | CRRKSSGGKGGSYSQAASSDSAQGSDV | TCTTAAGACTATTCAACCAAGGGTTGAAAAGTACTTATT | | |
| | SLTA (SEQ ID NO: RS C5p1full) | TGATGAGTCTGGTGAGTTTAAATTGTCAGAAGTAGGAC | | |
| | | CTGAGCATAGTCTTGCCGAATACTACATCTTCTTTGCAT | | |
| | | CATTTTATTATAAAAGATGTTTTCACACAACTGATCCTA | | |
| | | GTTTTCTGGGTAGGTACATGTCAGCATTATTTGCTGATG | | |
| | | ATTTAAACCAGTTAACTGGTTATCATACTGACTTTTCAA | | |
| | | GTGAAATCATAGGATACCAGCTTATGTGTCAACCTATAC | | |
| | | TGTTAGCAGAAGCTGAACTTGCAAAGAATGTGTCCTTA | | |
| | | ATTCTTGGAACTGTTTCTTGGAATTTGAAAAAGCGTTAT | | |
| | | TTATTATCAGCTGGTATTTTTGGTGCTATTACGGATGTTT | | |
| | | TCTACAAAGAAAACAGTTACAAAGTGCCAACAGACAAT | | |
| | | TATATAACCACTTACGCCCGAATGGCTGCCCCAAAAGA | | |
| | | AATTATCTTCTTAGAGGGAGAAACACTTTTTGGTGATGA | | |
| | | CACTGTGATAGAAGTGGCCATTATTTTGGCATCTTTTTC | | |
| | | TGCTTCCACAAGGAGGTCTGGAGCAGATTCCAACGGTA | | |
| | | CTATTACCGTTGAAGAGCTTAAAAAGCTCCTTGAACAAT | | |
| | | GGAACCTAGTAATAGGTTTCCTATTCCTTACATGGATTT | | |
| | | GTCTTCTACAATTTGCCTATGCCAACAGGAATAGGTTTT | | |
| | | TGTATATAATTAAGTTAATTTTCCTCTGGCTGTTATGGC | | |
| | | CAGTAACTTTAGCTTGTTTTGTGCTTGCTGCTGTTTACAG | | |
| | | AATAAATTGGATCACCGGTGGAATTGCTATCGCAAT | | |
| | | GGCTTGTCTTGTAGGCTTGATGTGGCTCAGCTACTTCAT | | |
| | | TGCTTCTTTCAGACTGTTTGCGCGTACGCGTTCCATGTG | | |
| | | GTCATTCAATCCAGAAACTAACATTCTTCTCAACGTGCC | | |
| | | ACTCCATGGCACTATTCTGACCAGACCGCTTCTAGAAAG | | |
| | | TGAACTCGTAATCGGAGCTGTGATCCTTCGTGGACATCT | | |
| | | TCGTATTGCTGGACACCATCTAGGACGCTGTGACATCAA | | |
| | | GGACCTGCCTAAAGAAATCACTGTTGCTACATCACGAA | | |
| | | CGCTTTCTTATTACAAATTGGGAGCTTCGCAGCGTGTAG | | |
| | | CAGGTGACTCAGGTTTTGCTGCATACAGTCGCTACAGG | | |
| | | ATTGGCAACTATAAATTAAACACAGACCATTCCAGTAG | | |
| | | CAGTGACAATATTGCTTTGCTTGTACAGGGTGGTTCTCT | | |
| | | TGGCGGAGGGGGTTCGGGAATCGTGGGAATTGTGGCAG | | |
| | | GACTGGCAGTGCTGGCCGTGGTGGTGATCGGAGCCGTG | | |
| | | GTGGCTACCGTGATGTGCAGACGGAAGTCCAGCGGAGG | | |
| | | CAAGGGCGGCAGCTACAGCCAGGCCGCCAGCTCTGATA | | |
| | | GCGCCCAGGGCAGCGACGTGTCACTGACAGCCTAGTAA | | |
| | | (SEQ ID NO: RS C5n1) | | |
| RS C6 | Named Segments: SP1-start GSS-N 99-Orf1ab linker1 1200-N 201-Orf1ab linker2 1200-M | | 507 | 1521 |
| 1200 | 201-Orf1ab linker3 1200-M 99-end GSS-MITD | | | |
| RS C6 | MRVMAPRTLILLLSGALALTETWAGSG | ATGAGAGTGATGGCCCCCAGAACCCTGATCCTGCTGCT | | |
| 1200 | GSGGGGSGGRMAGNGGDAALALLLLD | GTCTGGCGCCCTGGCCCTGACAGAGACATGGGCCGGAA | | |
| | RLNQLESKMSGKGQQQMRACMVTNNT | GCGGAGGTTCCGGAGGAGGCGGCAGTGGCGGCAGAAT | | |
| | FTLKVPHVGEIPVAYRKVLLKTIQPRVE | GGCTGGCAATGGCGGTGATGCTGCTCTTGCTTTGCTGCT | | |
| | KYLFDESGEFKLSEVGPEHSLAEYKMC | GCTTGACAGATTGAACCAGCTTGAGAGCAAAATGTCTG | | |
| | GYKHWPQIAQFAPSASAFFGMSRIGME | GTAAAGGCCAACAACAAATGAGAGCGTGCATGGTAACA | | |
| | VTPSGTWLTYTGAIKLDDKDPNFKDQV | AACAATACCTTCACACTCAAAGTCCCTCATGTGGGCGA | | |
| | ILLNKHIDAYKTFPARCATTDPSFLGRY | AATACCAGTGGCTTACCGCAAGGTTCTTCTTAAGACTAT | | |
| | MSALFADDLNQLTGYHTDFSSEIIGYQL | TCAACCAAGGGTTGAAAAGTACTTATTTGATGAGTCTG | | |
| | MCQPILLAEAELAKNVSLILGTVSWNLK | GTGAGTTTAAATTGTCAGAAGTAGGACCTGAGCATAGT | | |
| | KQGFAYANRNRFLYIIKLIFLWLLWPVT | CTTGCCGAATACAAAATGTGCGGATACAAACATTGGCC | | |
| | LACFVLAAVYRINWITGGIAIAMACLVG | GCAAATTGCACAATTTGCCCCCAGCGCTTCAGCGTTCTT | | |
| | LMWLSYFIASFRLFYRSYLLSAGIFGAIT | CGGAATGTCGCGCATTGGCATGGAAGTCACACCTTCGG | | |
| | DVFYKENSYKVPTDNYITTYARMAAPK | GAACGTGGTTGACCTACACAGGTGCCATCAAATTGGAT | | |
| | EIIFLEGETLFGDDTVIEVSMFNLGRCDI | GACAAAGATCCAAATTTCAAAGATCAAGTCATTTTGCT | | |
| | KDLPKEITVATSRTLSYYKLGASQRVAG | GAATAAGCATATTGACGCATACAAAACATTCCCAGCTC | | |
| | GSLGGGGSGIVGIVAGLAVLAVVVIGA | GGTGTGCAACAACTGATCCTAGTTTTCTGGGTAGGTACA | | |
| | VVATVMCRRKSSGGKGGSYSQAASSDS | TGTCAGCATTATTTGCTGATGATTTAAACCAGTTAACTG | | |
| | AQGSDVSLTA (SEQ ID NO: RS C6p1full) | GTTATCATACTGACTTTTCAAGTGAAATCATAGGATACC | | |
| | | AGCTTATGTGTCAACCTATACTGTTAGCAGAAGCTGAAC | | |
| | | TTGCAAAGAATGTGTCCTTAATTCTTGGAACTGTTTCTT | | |
| | | GGAATTTGAAAAAACAAGGTTTTGCCTATGCCAACAGG | | |
| | | AATAGGTTTTTGTATATAATTAAGTTAATTTTCCTCTGG | | |
| | | CTGTTATGGCCAGTAACTTTAGCTTGTTTTGTGCTTGCTG | | |
| | | CTGTTTACAGAATAAATTGGATCACCGGTGGAATTGCTA | | |
| | | TCGCAATGGCTTGTCTTGTAGGCTTGATGTGGCTCAGCT | | |
| | | ACTTCATTGCTTCTTTCAGACTGTTTTACCGCTCCTATTT | | |
| | | ATTATCAGCTGGTATTTTTGGTGCTATTACGGATGTTTTC | | |
| | | TACAAAGAAAACAGTTACAAAGTGCCAACAGACAATTA | | |
| | | TATAACCACTTACGCTCGGATGGCTGCCCCAAAAGAAA | | |
| | | TTATCTTCTTAGAGGGAGAAACACTTTTTGGTGATGACA | | |
| | | CTGTGATAGAAGTGTCTATGTTTAACCTAGGACGCTGTG | | |
| | | ACATCAAGGACCTGCCTAAAGAAATCACTGTTGCTACA | | |
| | | TCACGAACGCTTTCTTATTACAAATTGGGAGCTTCGCAG | | |
| | | CGTGTAGCAGGTGGTTCTCTTGGCGGAGGGGGTTCGGG | | |
| | | AATCGTGGGAATTGTGGCAGGACTGGCAGTGCTGGCCG | | |
| | | TGGTGGTGATCGGAGCCGTGGTGGCTACCGTGATGTGC | | |
| | | AGACGGAAGTCCAGCGGAGGCAAGGGCGGCAGCTACA | | |
| | | GCCAGGCCGCCAGCTCTGATAGCGCCCAGGGCAGCGAC | | |
| | | GTGTCACTGACAGCCTAGTAA (SEQ ID NO: RS C6n1) | | |
| RS C7 | | | | |
| 1500 M | Named Segments: SP1-start GSS-N 501-Orf1ab linker1 1500-M 201-Orf1ab linker2 1500-M | | | |
| chunks | 99-Orf1ab linker3 1500-end GSS-MITD | | | |
| RS C7 | MRVMAPRTLILLLSGALALTETWAGSG | ATGAGAGTGATGGCCCCCAGAACCCTGATCCTGCTGCT | 595 | 1785 |
| 1500 M | GSGGGGSGGSPARMAGNGGDAALALL | GTCTGGCGCCCTGGCCCTGACAGAGACATGGGCCGGAA | | |
| chunks | LLDRLNQLESKMSGKGQQQQGQTVTK | GCGGAGGTTCCGGAGGAGGCGGCAGTGGCGGCTCTCCT | | |
| | KSAAEASKKPRQKRTATKAYNVTQAFG | GCTAGAATGGCTGGCAATGGCGGTGATGCTGCTCTTGCT | | |
| | RRGPEQTQGNFGDQELIRQGTDYKHWP | TTGCTGCTGCTTGACAGATTGAACCAGCTTGAGAGCAA | | |
| | QIAQFAPSASAFFGMSRIGMEVTPSGTW | AATGTCTGGTAAAGGCCAACAACAACAAGGCCAAACTG | | |
| | LTYTGAIKLDDKDPNFKDQVILLNKHID | TCACTAAGAAATCTGCTGCTGAGGCTTCTAAGAAGCCTC | | |
| | AYKTFPPTEPKKDKFKAAMVTNNTFTL | GGCAAAAACGTACTGCCACTAAAGCATACAATGTAACA | | |
| | KVPHVGEIPVAYRKVLLKTIQPRVEKYL | CAAGCTTTCGGCAGACGTGGTCCAGAACAAACCCAAGG | | |
| | FDESGEFKLSEVGPEHSLAEYYIFFASFY | AAATTTTGGGGACCAGGAACTAATCAGACAAGGAACTG | | |
| | YKRNGFAYANRNRFLYIIKLIFLWLLWP | ATTACAAACATTGGCCGCAAATTGCACAATTTGCCCCCA | | |
| | VTLACFVLAAVYRINWITGGIAIAMACL | GCGCTTCAGCGTTCTTCGGAATGTCGCGCATTGGCATGG | | |
| | VGLMWLSYFIASFRLFYNSFHTTDPSFL | AAGTCACACCTTCGGGAACGTGGTTGACCTACACAGGT | | |
| | GRYMSALFADDLNQLTGYHTDFSSEIIG | GCCATCAAATTGGATGACAAAGATCCAAATTTCAAAGA | | |
| | YQLMCQPILLAEAELAKNVSLILGTVSW | TCAAGTCATTTTGCTGAATAAGCATATTGACGCATACAA | | |
| | NLKKNGLGRCDIKDLPKEITVATSRTLS | AACATTCCCACCAACAGAGCCTAAAAAGGACAAATTCA | | |
| | YYKLGASQRVAYRSYLLSAGIFGAITDV | AAGCCGCAATGGTAACAAACAATACCTTCACACTCAAA | | |
| | FYKENSYKVPTDNYITTYARMAAPKEII | GTCCCTCATGTGGGCGAAATACCAGTGGCTTACCGCAA | | |
| | FLEGETLFGDDTVIEVAIILASFSASTRR | GGTTCTTCTTAAGACTATTCAACCAAGGGTTGAAAAGTA | | |
| | RGGGSLGGGGSGIVGIVAGLAVLAVVV | CTTATTTGATGAGTCTGGTGAGTTTAAATTGTCAGAAGT | | |
| | IGAVVATVMCRRKSSGGKGGSYSQAAS | AGGACCTGAGCATAGTCTTGCCGAATACTACATCTTCTT | | |
| | SDSAQGSDVSLTA (SEQ ID NO: RS | TGCATCATTTTATTATAAGAGGAATGGTTTTGCCTATGC | | |
| | C7p1full) | CAACAGGAATAGGTTTTTGTATATAATTAAGTTAATTTT | | |
| | | CCTCTGGCTGTTATGGCCAGTAACTTTAGCTTGTTTTGT | | |
| | | GCTTGCTGCTGTTTACAGAATAAATTGGATCACCGGTGG | | |
| | | AATTGCTATCGCAATGGCTTGTCTTGTAGGCTTGATGTG | | |
| | | GCTCAGCTACTTCATTGCTTCTTTCAGACTGTTTTACAAT | | |
| | | TCTTTCCACACAACTGATCCTAGTTTTCTGGGTAGGTAC | | |
| | | ATGTCAGCATTATTTGCTGATGATTTAAACCAGTTAACT | | |
| | | GGTTATCATACTGACTTTTCAAGTGAAATCATAGGATAC | | |
| | | CAGCTTATGTGTCAACCTATACTGTTAGCAGAAGCTGAA | | |
| | | CTTGCAAAGAATGTGTCCTTAATTCTTGGAACTGTTTCT | | |
| | | TGGAATTTGAAGAAAAACGGACTAGGACGCTGTGACAT | | |
| | | CAAGGACCTGCCTAAAGAAATCACTGTTGCTACATCAC | | |
| | | GAACGCTTTCTTATTACAAATTGGGAGCTTCGCAGCGTG | | |
| | | TAGCATACCGATCCTACTTATTATCAGCTGGTATTTTTG | | |
| | | GTGCTATTACGGATGTTTTCTACAAAGAAAACAGTTACA | | |
| | | AAGTGCCAACAGACAATTATATAACCACTTACGCTCGG | | |
| | | ATGGCGGCCCCAAAAGAAATTATCTTCTTAGAGGGAGA | | |
| | | AACACTTTTTGGTGATGACACTGTGATAGAAGTGGCCAT | | |
| | | TATTTTGGCATCTTTTTCTGCTTCCACAAGGCGACGGGG | | |
| | | TGGTGGTTCTCTTGGCGGAGGGGGTTCGGGAATCGTGG | | |
| | | GAATTGTGGCAGGACTGGCAGTGCTGGCCGTGGTGGTG | | |
| | | ATCGGAGCCGTGGTGGCTACCGTGATGTGCAGACGGAA | | |
| | | GTCCAGCGGAGGCAAGGGCGGCAGCTACAGCCAGGCC | | |
| | | GCCAGCTCTGATAGCGCCCAGGGCAGCGACGTGTCACT | | |
| | | GACAGCCTAGTAA (SEQ ID NO: RS C7n1) | | |
| RS C8 | | | | |
| 1500 M | | | | |
| epitopes | Named Segments: SP1-start GSS-N 501-Orf1ab 1500 M epitopes-end GSS-MITD | | | |
| RS C8 | MRVMAPRTLILLLSGALALTETWAGSG | ATGAGAGTGATGGCCCCCAGAACCCTGATCCTGCTGCT | 588 | 1764 |
| 1500 M | GSGGGGSGGSPARMAGNGGDAALALL | GTCTGGCGCCCTGGCCCTGACAGAGACATGGGCCGGAA | | |
| epitopes | LLDRLNQLESKMSGKGQQQQGQTVTK | GCGGAGGTTCCGGAGGAGGCGGCAGTGGCGGCTCTCCT | | |
| | KSAAEASKKPRQKRTATKAYNVTQAFG | GCTAGAATGGCTGGCAATGGCGGTGATGCTGCTCTTGCT | | |
| | RRGPEQTQGNFGDQELIRQGTDYKHWP | TTGCTGCTGCTTGACAGATTGAACCAGCTTGAGAGCAA | | |
| | QIAQFAPSASAFFGMSRIGMEVTPSGTW | AATGTCTGGTAAAGGCCAACAACAACAAGGCCAAACTG | | |
| | LTYTGAIKLDDKDPNFKDQVILLNKHID | TCACTAAGAAATCTGCTGCTGAGGCTTCTAAGAAGCCTC | | |
| | AYKTFPPTEPKKDKFRACMVTNNTFTL | GGCAAAAACGTACTGCCACTAAAGCATACAATGTAACA | | |
| | KVPHVGEIPVAYRKVLLKTIQPRVEKYL | CAAGCTTTCGGCAGACGTGGTCCAGAACAAACCCAAGG | | |
| | FDESGEFKLSEVGPEHSLAEYYIFFASFY | AAATTTTGGGGACCAGGAACTAATCAGACAAGGAACTG | | |
| | YKRCFHTTDPSFLGRYMSALFADDLNQ | ATTACAAACATTGGCCGCAAATTGCACAATTTGCCCCCA | | |
| | LTGYHTDFSSEIIGYQLMCQPILLAEAEL | GCGCTTCAGCGTTCTTCGGAATGTCGCGCATTGGCATGG | | |
| | AKNVSLILGTVSWNLKKRYLLSAGIFGA | AAGTCACACCTTCGGGAACGTGGTTGACCTACACAGGT | | |
| | ITDVFYKENSYKVPTDNYITTYARMAA | GCCATCAAATTGGATGACAAAGATCCAAATTTCAAAGA | | |
| | PKEIIFLEGETLFGDDTVIEVAIILASFSAS | TCAAGTCATTTTGCTGAATAAGCATATTGACGCATACAA | | |
| | TRRRGKLLEQWNLVIGFNRNRFLYIIKLI | AACATTCCCACCAACAGAGCCTAAAAAGGACAAATTTC | | |
| | FLWLLWPVTLACFVLAAVYSELVIGAVI | GTGCGTGCATGGTAACAAACAATACCTTCACACTCAAA | | |
| | LRGHLRIAGHHLGRTVATSRTLSYYKL | GTCCCTCATGTGGGCGAAATACCAGTGGCTTACCGCAA | | |
| | GASQRVKRYSGLMWLSYFARYAGGSL | GGTTCTTCTTAAGACTATTCAACCAAGGGTTGAAAAGTA | | |
| | GGGGSGIVGIVAGLAVLAVVVIGAVVA | CTTATTTGATGAGTCTGGTGAGTTTAAATTGTCAGAAGT | | |
| | TVMCRRKSSGGKGGSYSQAASSDSAQG | AGGACCTGAGCATAGTCTTGCCGAATACTACATCTTCTT | | |
| | SDVSLTA (SEQ ID NO: RS C8p1full) | TGCATCATTTTATTATAAGCGCTGTTTTCACACAACTGA | | |
| | | TCCTAGTTTTCTGGGTAGGTACATGTCAGCATTATTTGC | | |
| | | TGATGATTTAAACCAGTTAACTGGTTATCATACTGACTT | | |
| | | TTCAAGTGAAATCATAGGATACCAGCTTATGTGTCAACC | | |
| | | TATACTGTTAGCAGAAGCTGAACTTGCAAAGAATGTGT | | |
| | | CCTTAATTCTTGGAACTGTTTCTTGGAATTTGAAAAAAC | | |
| | | GTTACTTATTATCAGCTGGTATTTTTGGTGCTATTACGG | | |
| | | ATGTTTTCTACAAAGAAAACAGTTACAAAGTGCCAACA | | |
| | | GACAATTATATAACCACTTACGCGCGAATGGCAGCCCC | | |
| | | AAAAGAAATTATCTTCTTAGAGGGAGAAACACTTTTTG | | |
| | | GTGATGACACTGTGATAGAAGTGGCCATTATTTTGGCAT | | |
| | | CTTTTTCTGCTTCCACACGCAGGCGCGGAAAGCTCCTTG | | |
| | | AACAATGGAACCTAGTAATAGGTTTCAACAGGAATAGG | | |
| | | TTTTTGTATATAATTAAGTTAATTTTCCTCTGGCTGTTAT | | |
| | | GGCCAGTAACTTTAGCTTGTTTTGTGCTTGCTGCTGTTTA | | |
| | | CAGTGAACTCGTAATCGGAGCTGTGATCCTTCGTGGAC | | |
| | | ATCTTCGTATTGCTGGACACCATCTAGGACGCACTGTTG | | |
| | | CTACATCACGAACGCTTTCTTATTACAAATTGGGAGCTT | | |
| | | CGCAGCGTGTAAAACGCTACAGTGGCTTGATGTGGCTC | | |
| | | AGCTACTTCGCGCGTTATGCCGGTGGTTCTCTTGGCGGA | | |
| | | GGGGGTTCGGGAATCGTGGGAATTGTGGCAGGACTGGC | | |
| | | AGTGCTGGCCGTGGTGGTGATCGGAGCCGTGGTGGCTA | | |
| | | CCGTGATGTGCAGACGGAAGTCCAGCGGAGGCAAGGGC | | |
| | | GGCAGCTACAGCCAGGCCGCCAGCTCTGATAGCGCCCA | | |
| | | GGGCAGCGACGTGTCACTGACAGCCTAGTAA (SEQ ID | | |
| | | NO: RS C8n1) | | |

TABLE 15
Modified String Sequences Group 2

| Con- | | | |
|---|---|---|---|
| struct | Name | Amino Acid sequence | Nucleotide sequence |
| name | | | |
| RS-C5 | dEarI- | | ATTCTTCTGGTCCCCACAGACTCAGAGAGAACCC |
| | hAg | | |
| RS-C5 | Kozak | | GCCACC |
| RS-C5 | SEC | MFVFLVLLPLVSSQCVNLT | ATGTTTGTTTTTCTTGTTTTACTTCCTTTAGTCTCTTCTCAGTGTGTCAAT |
| | | | TTGACA |
| RS-C5 | RS-C5 | GGSGGGGSGGKDLSPRWYFYYLGT | GGCGGCTCTGGAGGAGGCGGCTCCGGAGGCAAAGATCTGAGCCCAAG |
| | | GPEAGLPYGANKDGIIWVATEGALN | ATGGTACTTTTACTACCTGGGAACAGGACCTGAAGCTGGACTGCCTTA |
| | | TPKDHIGTRNPANNAAIVLQLPQGT | TGGAGCAAATAAAGATGGAATCATCTGGGTGGCAACAGAAGGAGCAC |
| | | TLPKGFYAEGSRGGSQASSRSSSRSR | TGAATACACCAAAAGATCACATTGGAACCAGAAATCCAGCAAATAAT |
| | | NSSRNSTPGSSRGTSPARMAGNGGD | GCAGCAATCGTGCTGCAGCTGCCACAGGGAACAACACTGCCAAAAGG |
| | | AALALLLLDRLNQLESKMSGKGQQ | ATTTTACGCAGAAGGAAGCAGAGGAGGAAGCCAGGCAAGCAGCAGAA |
| | | QQGQTVTKKSAAEASKKPRQKRTA | GCAGCAGCAGAAGCAGAAATAGCAGCAGAAATAGCACACCTGGAAGC |
| | | TKAYNVTQAFGRRGPEQTQGNFGD | AGCAGAGGAACAAGCCCTGCAAGAATGGCAGGAAATGGAGGAGATGC |
| | | QELIRQGTDYKHWPQIAQFAPSASA | AGCACTGGCACTGCTGCTGCTGGATAGACTGAATCAGCTGGAAAGCAA |
| | | FFGMSRIGMEVTPSGTWLTYTGAIK | AATGAGCGGAAAAGGACAGCAGCAGCAGGGACAGACAGTGACAAAG |
| | | LDDKDPNFKDQVILLNKHIDAYKTF | AAAAGCGCTGCTGAAGCTTCCAAGAAACCAAGACAGAAAAGAACAGC |
| | | PPTEPKKDKKKKADETQALPQRQK | AACAAAAGCATACAATGTGACACAGGCATTTGGAAGGAGAGGACCTG |
| | | KQQTVTLLPAADLDDFSKQLQQSMS | AACAGACCCAGGGAAATTTTGGAGATCAGGAACTGATCAGACAGGGA |
| | | SADSTQAFRACMVTNNTFTLKVPHV | ACCGATTACAAACACTGGCCTCAGATTGCTCAGTTTGCTCCTTCTGCTT |
| | | GEIPVAYRKVLLKTIQPRVEKYLFDE | CTGCTTTCTTTGGAATGTCCAGAATTGGAATGGAAGTGACACCTTCTGG |
| | | SGEFKLSEVGPEHSLAEYYIFFASFY | AACATGGCTGACATACACAGGAGCAATCAAACTGGATGATAAAGATC |
| | | YKRCFHTTDPSFLGRYMSALFADDL | CAAATTTTAAAGATCAGGTGATTCTGCTGAATAAACACATTGATGCTT |
| | | NQLTGYHTDFSSEIIGYQLMCQPILL | ACAAAACATTTCCACCAACAGAACCAAAGAAAGATAAAAAGAAGAAA |
| | | AEAELAKNVSLILGTVSWNLKKRYL | GCTGATGAAACACAGGCTCTGCCTCAGAGACAGAAGAAACAGCAGAC |
| | | LSAGIFGAITDVFYKENSYKVPTDNY | AGTGACACTGCTGCCTGCTGCTGATCTGGATGATTTTTCCAAACAGCTG |
| | | ITTYARMAAPKEIIFLEGETLFGDDT | CAGCAGTCCATGTCCTCCGCTGATTCCACACAGGCTTTTAGAGCTTGCA |
| | | VIEVAIILASFSASTRRSGADSNGTIT | TGGTGACAAACAACACATTTACACTGAAAGTGCCTCATGTGGGAGAAA |
| | | VEELKKLLEQWNLVIGFLFLTWICLL | TTCCTGTGGCTTACAGAAAAGTGCTGCTGAAAACAATTCAACCAAGAG |
| | | QFAYANRNRFLYIIKLIFLWLLWPVT | TGGAAAAATACCTGTTTGATGAATCTGGAGAATTTAAACTTTCTGAAG |
| | | LACFVLAAVYRINWITGGIAIAMAC | TGGGACCTGAACACTCTCTTGCTGAATACTACATTTTCTTTGCTTCTTTT |
| | | LVGLMWLSYFIASFRLFARTRSMWS | TACTATAAAAGATGTTTTCACACAACTGATCCATCTTTTCTGGGAAGAT |
| | | FNPETNILLNVPLHGTILTRPLLESEL | ACATGTCTGCTCTTTTTGCTGATGATTTAAATCAGTTAACAGGATATCA |
| | | VIGAVILRGHLRIAGHHLGRCDIKDL | CACAGATTTTTCTTCTGAAATCATTGGATACCAGTTGATGTGTCAACCA |
| | | PKEITVATSRTLSYYKLGASQRVAG | ATTTTGTTGGCTGAAGCTGAATTGGCAAAAAACGTGTCTTTGATTCTTG |
| | | DSGFAAYSRYRIGNYKLNTDHSSSS | GAACAGTTTCTTGGAATCTTAAAAAGAGATACTTATTATCTGCTGGAAT |
| | | DNIALLVQGGSGGGGSGG (SEQ ID | TTTTGGAGCTATCACAGATGTGTTTTACAAAGAAAACTCATACAAAGT |
| | | NO: RS C5p2) | GCCAACAGACAATTACATAACAACATACGCCAGAATGGCTGCTCCAAA |
| | | | AGAAATCATCTTCCTTGAAGGAGAAACACTTTTTGGAGATGATACAGT |
| | | | GATTGAAGTGGCAATTATTCTGGCATCTTTTTCTGCATCCACAAGAAGA |
| | | | TCTGGAGCTGATTCCAATGGAACAATTACAGTGGAAGAACTGAAAAAG |
| | | | CTGCTGGAACAGTGGAATCTGGTGATTGGATTTCTGTTTCTGACATGGA |
| | | | TTTGTCTGCTGCAGTTTGCTTACGCTAATAGAAATAGATTTCTGTACAT |
| | | | TATTAAACTGATTTTTCTGTGGCTGCTGTGGCCTGTGACACTGGCTTGC |
| | | | TTTGTGCTGGCTGCTGTGTACAGAATTAATTGGATTACAGGAGGAATT |
| | | | GCAATTGCAATGGCATGCCTGGTGGGACTGATGTGGCTGTCTTACTTTA |
| | | | TTGCTTCTTTTAGACTGTTTGCAAGAACAAGATCTATGTGGTCTTTTAA |
| | | | TCCTGAAACAAATATTCTGCTGAATGTGCCTCTGCACGGAACAATTCTG |
| | | | ACAAGACCTCTGCTGGAATCTGAACTGGTGATTGGAGCTGTGATTCTG |
| | | | AGAGGACACCTGAGAATTGCTGGACACCACCTGGGAAGATGCGATATT |
| | | | AAAGATCTGCCAAAAGAAATTACAGTGGCAACAAGCAGAACACTGAG |
| | | | CTACTACAAACTGGGAGCTTCCCAAAGAGTGGCTGGAGATAGCGGATT |
| | | | TGCTGCTTACAGCAGATACAGAATTGGAAATTACAAACTGAATACCGA |
| | | | TCACAGCTCATCTTCTGATAATATTGCTCTGCTGGTGCAGGGAGGATCC |
| | | | GGTGGTGGCGGCAGCGGCGGC (SEQ ID NO: RS C5n2) |
| RS-C5 | TM | EQYIKWPWYIWLGFIAGLIAIVMVTI | GAACAGTACATTAAATGGCCTTGGTACATTTGGCTTGGATTTATTGCAG |
| | | MLCCMTSCCSCLKGCCSCGSCCKFD | GATTAATTGCAATTGTGATGGTGACAATTATGTTATGTTGTATGACATC |
| | | EDDSEPVLKGVKLHYT** | ATGTTGTTCTTGTTTAAAAGGATGTTGTTCTTGTGGAAGCTGTTGTAAA |
| | | | TTTGATGAAGATGATTCTGAACCTGTGTTAAAAGGAGTGAAATTGCAT |
| | | | TACACATGATGA |
| RS-C5 | | | CTCGAG |
| RS-C5 | F element | | CTGGTACTGCATGCACGCAATGCTAGCTGCCCCTTTCCCGTCCTGGGTA |
| | | | CCCCGAGTCTCCCCCGACCTCGGGTCCCAGGTATGCTCCCACCTCCACC |
| | | | TGCCCCACTCACCACCTCTGCTAGTTCCAGACACCTCC |
| RS-C5 | I element | | CAAGCACGCAGCAATGCAGCTCAAAACGCTTAGCCTAGCCACACCCCC |
| | | | ACGGGAAACAGCAGTGATTAACCTTTAGCAATAAACGAAAGTTTAACT |
| | | | AAGCTATACTAACCCCAGGGTTGGTCAATTTCGTGCCAGCCACACC |
| RS-C5 | | | CTGGAGCTAGC |
| RS-C5 | PolyA | | AAAAAAAAAAAAAAAAAAAAAAAAAAAAAAGCATATGACTAAAAAA |
| | | | AAAAAAAAAAAAAAAAAAAAAAAAAAAAAAAAAAAAAAAAAAAAA |
| | | | AAAAAAAAAAAAAAAAAAA |
| RS-C5 | Full | MFVFLVLLPLVSSQCVNLTGGSGGG | ATTCTTCTGGTCCCCACAGACTCAGAGAGAACCCGCCACCATGTTTGTT |
| | Sequence | GSGGKDLSPRWYFYYLGTGPEAGLP | TTTCTTGTTTTACTTCCTTTAGTCTCTTCTCAGTGTGTCAATTTGACAGG |
| | | YGANKDGIIWVATEGALNTPKDHIG | CGGCTCTGGAGGAGGCGGCTCCGGAGGCAAAGATCTGAGCCCAAGAT |
| | | TRNPANNAAIVLQLPQGTTLPKGFY | GGTACTTTTACTACCTGGGAACAGGACCTGAAGCTGGACTGCCTTATG |
| | | AEGSRGGSQASSRSSSRSRNSSRNST | GAGCAAATAAAGATGGAATCATCTGGGTGGCAACAGAAGGAGCACTG |
| | | PGSSRGTSPARMAGNGGDAALALLL | AATACACCAAAAGATCACATTGGAACCAGAAATCCAGCAAATAATGC |
| | | LDRLNQLESKMSGKGQQQQGQTVT | AGCAATCGTGCTGCAGCTGCCACAGGGAACAACACTGCCAAAAGGATT |
| | | KKSAAEASKKPRQKRTATKAYNVT | TTACGCAGAAGGAAGCAGAGGAGGAAGCCAGGCAAGCAGCAGAAGC |
| | | QAFGRRGPEQTQGNFGDQELIRQGT | AGCAGCAGAAGCAGAAATAGCAGCAGAAATAGCACACCTGGAAGCAG |
| | | DYKHWPQIAQFAPSASAFFGMSRIG | CAGAGGAACAAGCCCTGCAAGAATGGCAGGAAATGGAGGAGATGCAG |
| | | MEVTPSGTWLTYTGAIKLDDKDPNF | CACTGGCACTGCTGCTGCTGGATAGACTGAATCAGCTGGAAAGCAAAA |
| | | KDQVILLNKHIDAYKTFPPTEPKKD | TGAGCGGAAAAGGACAGCAGCAGCAGGGACAGACAGTGACAAAGAA |
| | | KKKKADETQALPQRQKKQQTVTLL | AAGCGCTGCTGAAGCTTCCAAGAAACCAAGACAGAAAAGAACAGCAA |
| | | PAADLDDFSKQLQQSMSSADSTQAF | CAAAAGCATACAATGTGACACAGGCATTTGGAAGGAGAGGACCTGAA |
| | | RACMVTNNTFTLKVPHVGEIPVAYR | CAGACCCAGGGAAATTTTGGAGATCAGGAACTGATCAGACAGGGAAC |
| | | KVLLKTIQPRVEKYLFDESGEFKLSE | CGATTACAAACACTGGCCTCAGATTGCTCAGTTTGCTCCTTCTGCTTCT |
| | | VGPEHSLAEYYIFFASFYYKRCFHTT | GCTTTCTTTGGAATGTCCAGAATTGGAATGGAAGTGACACCTTCTGGA |
| | | DPSFLGRYMSALFADDLNQLTGYHT | ACATGGCTGACATACACAGGAGCAATCAAACTGGATGATAAAGATCC |
| | | DFSSEIIGYQLMCQPILLAEAELAKN | AAATTTTAAAGATCAGGTGATTCTGCTGAATAAACACATTGATGCTTA |
| | | VSLILGTVSWNLKKRYLLSAGIFGAI | CAAAACATTTCCACCAACAGAACCAAAGAAAGATAAAAAGAAGAAAG |
| | | TDVFYKENSYKVPTDNYITTYARMA | CTGATGAAACACAGGCTCTGCCTCAGAGACAGAAGAAACAGCAGACA |
| | | APKEIIFLEGETLFGDDTVIEVAIILAS | GTGACACTGCTGCCTGCTGCTGATCTGGATGATTTTTCCAAACAGCTGC |
| | | FSASTRRSGADSNGTITVEELKKLLE | AGCAGTCCATGTCCTCCGCTGATTCCACACAGGCTTTTAGAGCTTGCAT |
| | | QWNLVIGFLFLTWICLLQFAYANRN | GGTGACAAACAACACATTTACACTGAAAGTGCCTCATGTGGGAGAAAT |
| | | RFLYIIKLIFLWLLWPVTLACFVLAA | TCCTGTGGCTTACAGAAAAGTGCTGCTGAAAACAATTCAACCAAGAGT |
| | | VYRINWITGGIAIAMACLVGLMWLS | GGAAAAATACCTGTTTGATGAATCTGGAGAATTTAAACTTTCTGAAGT |
| | | YFIASFRLFARTRSMWSFNPETNILL | GGGACCTGAACACTCTCTTGCTGAATACTACATTTTCTTTGCTTCTTTTT |
| | | NVPLHGTILTRPLLESELVIGAVILRG | ACTATAAAAGATGTTTTCACACAACTGATCCATCTTTTCTGGGAAGATA |
| | | HLRIAGHHLGRCDIKDLPKEITVATS | CATGTCTGCTCTTTTTGCTGATGATTTAAATCAGTTAACAGGATATCAC |
| | | RTLSYYKLGASQRVAGDSGFAAYSR | ACAGATTTTTCTTCTGAAATCATTGGATACCAGTTGATGTGTCAACCAA |
| | | YRIGNYKLNTDHSSSSDNIALLVQG | TTTTGTTGGCTGAAGCTGAATTGGCAAAAAACGTGTCTTTGATTCTTGG |
| | | GSGGGGSGGEQYIKWPWYIWLGFIA | AACAGTTTCTTGGAATCTTAAAAAGAGATACTTATTATCTGCTGGAATT |
| | | GLIAIVMVTIMLCCMTSCCSCLKGC | TTTGGAGCTATCACAGATGTGTTTTACAAAGAAAACTCATACAAAGTG |
| | | CSCGSCCKFDEDDSEPVLKGVKLHY | CCAACAGACAATTACATAACAACATACGCCAGAATGGCTGCTCCAAAA |
| | | T** (SEQ ID NO: RS C5p2full) | GAAATCATCTTCCTTGAAGGAGAAACACTTTTTGGAGATGATACAGTG |
| | | | ATTGAAGTGGCAATTATTCTGGCATCTTTTTCTGCATCCACAAGAAGAT |
| | | | CTGGAGCTGATTCCAATGGAACAATTACAGTGGAAGAACTGAAAAAG |
| | | | CTGCTGGAACAGTGGAATCTGGTGATTGGATTTCTGTTTCTGACATGGA |
| | | | TTTGTCTGCTGCAGTTTGCTTACGCTAATAGAAATAGATTTCTGTACAT |
| | | | TATTAAACTGATTTTTCTGTGGCTGCTGTGGCCTGTGACACTGGCTTGC |
| | | | TTTGTGCTGGCTGCTGTGTACAGAATTAATTGGATTACAGGAGGAATT |
| | | | GCAATTGCAATGGCATGCCTGGTGGGACTGATGTGGCTGTCTTACTTTA |
| | | | TTGCTTCTTTTAGACTGTTTGCAAGAACAAGATCTATGTGGTCTTTTAA |
| | | | TCCTGAAACAAATATTCTGCTGAATGTGCCTCTGCACGGAACAATTCTG |
| | | | ACAAGACCTCTGCTGGAATCTGAACTGGTGATTGGAGCTGTGATTCTG |
| | | | AGAGGACACCTGAGAATTGCTGGACACCACCTGGGAAGATGCGATATT |
| | | | AAAGATCTGCCAAAAGAAATTACAGTGGCAACAAGCAGAACACTGAG |
| | | | CTACTACAAACTGGGAGCTTCCCAAAGAGTGGCTGGAGATAGCGGATT |
| | | | TGCTGCTTACAGCAGATACAGAATTGGAAATTACAAACTGAATACCGA |
| | | | TCACAGCTCATCTTCTGATAATATTGCTCTGCTGGTGCAGGGAGGATCC |
| | | | GGTGGTGGCGGCAGCGGCGGCGAACAGTACATTAAATGGCCTTGGTAC |
| | | | ATTTGGCTTGGATTTATTGCAGGATTAATTGCAATTGTGATGGTGACAA |
| | | | TTATGTTATGTTGTATGACATCATGTTGTTCTTGTTTAAAAGGATGTTGT |
| | | | TCTTGTGGAAGCTGTTGTAAATTTGATGAAGATGATTCTGAACCTGTGT |
| | | | TAAAAGGAGTGAAATTGCATTACACATGATGACTCGAGCTGGTACTGC |
| | | | ATGCACGCAATGCTAGCTGCCCCTTTCCCGTCCTGGGTACCCCGAGTCT |
| | | | CCCCCGACCTCGGGTCCCAGGTATGCTCCCACCTCCACCTGCCCCACTC |
| | | | ACCACCTCTGCTAGTTCCAGACACCTCCCAAGCACGCAGCAATGCAGC |
| | | | TCAAAACGCTTAGCCTAGCCACACCCCCACGGGAAACAGCAGTGATTA |
| | | | ACCTTTAGCAATAAACGAAAGTTTAACTAAGCTATACTAACCCCAGGG |
| | | | TTGGTCAATTTCGTGCCAGCCACACCCTGGAGCTAGCAAAAAAAAAAA |
| | | | AAAAAAAAAAAAAAAAAAAGCATATGACTAAAAAAAAAAAAAAAAA |
| | | | AAAAAAAAAAAAAAAAAAAAAAAAAAAAAAAAAAAAAAAAAAAAA |
| | | | AAAAAAAA (SEQ ID NO: RS C5n2full) |
| | | | ATGAGAGTGATGGCCCCCAGAACCCTGATCCTGCTGCTGTCTGGCGCC |
| | | | CTGGCCCTGACAGAGACATGGGCCGGAAGCGGCGGCTCTGGAGGAGG |
| | | | CGGCTCCGGAGGCAAAGATCTGAGCCCAAGATGGTACTTTTACTACCT |
| | | | GGGAACAGGACCTGAAGCTGGACTGCCTTATGGAGCAAATAAAGATG |
| | | | GAATCATCTGGGTGGCAACAGAAGGAGCACTGAATACACCAAAAGAT |
| | | | CACATTGGAACCAGAAATCCAGCAAATAATGCAGCAATCGTGCTGCAG |
| | | | CTGCCACAGGGAACAACACTGCCAAAAGGATTTTACGCAGAAGGAAG |
| | | | CAGAGGAGGAAGCCAGGCAAGCAGCAGAAGCAGCAGCAGAAGCAGA |
| | | | AATAGCAGCAGAAATAGCACACCTGGAAGCAGCAGAGGAACAAGCCC |
| | | | TGCAAGAATGGCAGGAAATGGAGGAGATGCAGCACTGGCACTGCTGC |
| | | | TGCTGGATAGACTGAATCAGCTGGAAAGCAAAATGAGCGGAAAAGGA |
| | | | CAGCAGCAGCAGGGACAGACAGTGACAAAGAAAAGCGCTGCTGAAGC |
| | | | TTCCAAGAAACCAAGACAGAAAAGAACAGCAACAAAAGCATACAATG |
| | | | TGACACAGGCATTTGGAAGGAGAGGACCTGAACAGACCCAGGGAAAT |
| | | | TTTGGAGATCAGGAACTGATCAGACAGGGAACCGATTACAAACACTGG |
| | | | CCTCAGATTGCTCAGTTTGCTCCTTCTGCTTCTGCTTTCTTTGGAATGTC |
| | | | CAGAATTGGAATGGAAGTGACACCTTCTGGAACATGGCTGACATACAC |
| | | | AGGAGCAATCAAACTGGATGATAAAGATCCAAATTTTAAAGATCAGGT |
| | | | GATTCTGCTGAATAAACACATTGATGCTTACAAAACATTTCCACCAAC |
| | | | AGAACCAAAGAAAGATAAAAAGAAGAAAGCTGATGAAACACAGGCTC |
| | | | TGCCTCAGAGACAGAAGAAACAGCAGACAGTGACACTGCTGCCTGCTG |
| | | | CTGATCTGGATGATTTTTCCAAACAGCTGCAGCAGTCCATGTCCTCCGC |
| | | | TGATTCCACACAGGCTTTTAGAGCTTGCATGGTGACAAACAACACATT |
| | | | TACACTGAAAGTGCCTCATGTGGGAGAAATTCCTGTGGCTTACAGAAA |
| | | | AGTGCTGCTGAAAACAATTCAACCAAGAGTGGAAAAATACCTGTTTGA |
| | | | TGAATCTGGAGAATTTAAACTTTCTGAAGTGGGACCTGAACACTCTCTT |
| | | | GCTGAATACTACATTTTCTTTGCTTCTTTTTACTATAAAAGATGTTTTCA |
| | | | CACAACTGATCCATCTTTTCTGGGAAGATACATGTCTGCTCTTTTTGCT |
| | | | GATGATTTAAATCAGTTAACAGGATATCACACAGATTTTTCTTCTGAAA |
| | | | TCATTGGATACCAGTTGATGTGTCAACCAATTTTGTTGGGTGAAGCAG |
| | | | AATTGGCAAAAAACGTGTCTTTGATTCTTGGAACAGTTTCTTGGAATCT |
| | | | TAAAAAGAGATACTTATTATCTGCTGGAATTTTTGGAGCTATCACAGAT |
| | | | GTGTTTTACAAAGAAAACTCATACAAAGTGCCAACAGACAATTACATA |
| | | | ACAACATACGCCAGAATGGCTGCTCCAAAAGAAATCATCTTCCTTGAA |
| | | | GGAGAAACACTTTTTGGAGATGATACAGTGATTGAAGTGGCAATTATT |
| | | | CTGGCATCTTTTTCTGCATCCACAAGAAGATCTGGAGCTGATTCCAATG |
| | | | GAACAATTACAGTGGAAGAACTGAAAAAGCTGCTGGAACAGTGGAAT |
| | | | CTGGTGATTGGATTTCTGTTTCTGACATGGATTTGTCTGCTGCAGTTTG |
| | | | CTTACGCTAATAGAAATAGATTTCTGTACATTATTAAACTGATTTTTCT |
| | | | GTGGCTGCTGTGGCCTGTGACACTGGCTTGCTTTGTGCTGGCTGCTGTG |
| | | | TACAGAATTAATTGGATTACAGGAGGAATTGCAATTGCAATGGCATGC |
| | | | CTGGTGGGACTGATGTGGCTGTCTTACTTTATTGCTTCTTTTAGACTGTT |
| | | | TGCAAGAACAAGATCTATGTGGTCTTTTAATCCTGAAACAAATATTCTG |
| | | | CTGAATGTGCCTCTGCACGGAACAATTCTGACAAGACCTCTGCTGGAA |
| | | | TCTGAACTGGTGATTGGAGCTGTGATTCTGAGAGGACACCTGAGAATT |
| | | | GCTGGACACCACCTGGGAAGATGCGATATTAAAGATCTGCCAAAAGA |
| | | | AATTACAGTGGCAACAAGCAGAACACTGAGCTACTACAAACTGGGAG |
| | | | CTTCCCAAAGAGTGGCTGGAGATAGCGGATTTGCTGCTTACAGCAGAT |
| | | | ACAGAATTGGAAATTACAAACTGAATACCGATCACAGCTCATCTTCTG |
| | | | ATAATATTGCTCTGCTGGTGCAGGGTGGTTCTCTTGGCGGAGGGGGTTC |
| | | | GGGAATCGTGGGAATTGTGGCAGGACTGGCAGTGCTGGCCGTGGTGGT |
| | | | GATCGGAGCCGTGGTGGCTACCGTGATGTGCAGACGGAAGTCCAGCGG |
| | | | AGGCAAGGGCGGCAGCTACAGCCAGGCCGCCAGCTCTGATAGCGCCC |
| | | | AGGGCAGCGACGTGTCACTGACAGCCTAGTAA (SEQ ID NO: RS C5n3) |
| RS-C6 | dEarI- | | ATTCTTCTGGTCCCCACAGACTCAGAGAGAACCC |
| | hAg | | |
| RS-C6 | Kozak | | GCCACC |
| RS-C6 | SEC | MFVFLVLLPLVSSQCVNLT | ATGTTTGTTTTTCTTGTTTTACTTCCTTTAGTCTCTTCTCAGTGTGTCAAT |
| | | | TTGACA |
| RS-C6 | RS-C6 | GGSGGGGSGGRMAGNGGDAALAL | GGCGGCTCTGGAGGAGGCGGCTCCGGAGGCAGAATGGCTGGAAATGG |
| | | LLLDRLNQLESKMSGKGQQQMRAC | AGGAGATGCTGCTCTGGCTCTGCTGCTGCTGGATAGACTGAATCAGCT |
| | | MVTNNTFTLKVPHVGEIPVAYRKVL | GGAATCAAAAATGTCTGGAAAAGGACAGCAACAGATGAGAGCTTGCA |
| | | LKTIQPRVEKYLFDESGEFKLSEVGP | TGGTGACAAACAACACATTTACACTGAAAGTGCCTCATGTGGGAGAAA |
| | | EHSLAEYKMCGYKHWPQIAQFAPS | TTCCTGTGGCTTACAGAAAAGTGCTGCTGAAAACAATTCAACCAAGAG |
| | | ASAFFGMSRIGMEVTPSGTWLTYTG | TGGAAAAATACCTGTTTGATGAATCTGGAGAATTTAAACTTTCTGAAG |
| | | AIKLDDKDPNFKDQVILLNKHIDAY | TGGGACCTGAACACTCTCTTGCTGAATACAAAATGTGCGGATACAAAC |
| | | KTFPARCATTDPSFLGRYMSALFAD | ACTGGCCTCAGATTGCTCAGTTTGCTCCTTCTGCTTCTGCTTTCTTTGGA |
| | | DLNQLTGYHTDFSSEIIGYQLMCQPI | ATGAGCAGAATTGGAATGGAAGTGACACCTTCTGGAACATGGCTGACA |
| | | LLAEAELAKNVSLILGTVSWNLKKQ | TACACAGGAGCAATCAAACTGGATGATAAAGATCCAAACTTTAAAGAT |
| | | GFAYANRNRFLYIIKLIFLWLLWPVT | CAGGTGATTCTGCTGAACAAACACATTGATGCTTACAAAACATTTCCT |
| | | LACFVLAAVYRINWITGGIAIAMAC | GCTAGATGTGCTACAACTGATCCTTCTTTTCTGGGAAGATACATGTCTG |
| | | LVGLMWLSYFIASFRLFYRSYLLSA | CTCTTTTTGCTGATGATTTAAATCAGTTAACAGGATATCACACAGATTT |
| | | GIFGAITDVFYKENSYKVPTDNYITT | TTCTTCTGAAATCATTGGATACCAGTTGATGTGTCAACCAATTTTGTTG |
| | | YARMAAPKEIIFLEGETLFGDDTVIE | GCTGAAGCTGAATTGGCAAAAAACGTGTCTTTGATTCTTGGAACAGTT |
| | | VSMFNLGRCDIKDLPKEITVATSRTL | TCTTGGAATCTTAAAAAACAAGGATTTGCATACGCAAATAGAAATAGA |
| | | SYYKLGASQRVAGGSGGGGSGG | TTTCTGTACATTATTAAACTGATTTTTCTGTGGCTGCTGTGGCCTGTGAC |
| | | (SEQ ID NO: RS C6p2) | ACTGGCTTGCTTTGTGCTGGCTGCTGTGTACAGAATAAATTGGATAACA |
| | | | GGAGGAATTGCAATTGCAATGGCATGCTTGGTGGGATTGATGTGGTTG |
| | | | TCTTACTTTATTGCTTCTTTTAGACTGTTTTACAGATCCTACTTATTATC |
| | | | TGCTGGAATTTTTGGAGCTATCACAGATGTGTTTTACAAAGAAAACTC |
| | | | ATACAAAGTGCCAACAGACAATTACATAACAACATACGCTAGAATGGC |
| | | | TGCTCCAAAAGAAATCATCTTCCTTGAAGGAGAAACACTTTTTGGAGA |
| | | | TGATACAGTGATTGAAGTGTCCATGTTTAATCTGGGAAGATGCGATAT |
| | | | TAAAGATCTGCCAAAAGAAATTACAGTGGCAACAAGCAGAACACTGA |
| | | | GCTACTACAAACTGGGAGCAAGCCAGAGAGTGGCAGGAGGATCCGGT |
| | | | GGTGGCGGCAGCGGCGGC (SEQ ID NO: RS C6n2) |
| RS-C6 | TM | EQYIKWPWYIWLGFIAGLIAIVMVTI | GAACAGTACATTAAATGGCCTTGGTACATTTGGCTTGGATTTATTGCAG |
| | | MLCCMTSCCSCLKGCCSCGSCCKFD | GATTAATTGCAATTGTGATGGTGACAATTATGTTATGTTGTATGACATC |
| | | EDDSEPVLKGVKLHYT** | ATGTTGTTCTTGTTTAAAAGGATGTTGTTCTTGTGGAAGCTGTTGTAAA |
| | | | TTTGATGAAGATGATTCTGAACCTGTGTTAAAAGGAGTGAAATTGCAT |
| | | | TACACATGATGA |
| RS-C6 | | | CTCGAG |
| RS-C6 | F element | | CTGGTACTGCATGCACGCAATGCTAGCTGCCCCTTTCCCGTCCTGGGTA |
| | | | CCCCGAGTCTCCCCCGACCTCGGGTCCCAGGTATGCTCCCACCTCCACC |
| | | | TGCCCCACTCACCACCTCTGCTAGTTCCAGACACCTCC |
| RS-C6 | I element | | CAAGCACGCAGCAATGCAGCTCAAAACGCTTAGCCTAGCCACACCCCC |
| | | | ACGGGAAACAGCAGTGATTAACCTTTAGCAATAAACGAAAGTTTAACT |
| | | | AAGCTATACTAACCCCAGGGTTGGTCAATTTCGTGCCAGCCACACC |
| RS-C6 | | | CTGGAGCTAGC |
| RS-C6 | PolyA | | AAAAAAAAAAAAAAAAAAAAAAAAAAAAAAGCATATGACTAAAAAA |
| | | | AAAAAAAAAAAAAAAAAAAAAAAAAAAAAAAAAAAAAAAAAAAAA |
| | | | AAAAAAAAAAAAAAAAAAA |
| RS-C6 | FullSeq | MFVFLVLLPLVSSQCVNLTGGSGGG | ATTCTTCTGGTCCCCACAGACTCAGAGAGAACCCGCCACCATGTTTGTT |
| | | GSGGRMAGNGGDAALALLLLDRLN | TTTCTTGTTTTACTTCCTTTAGTCTCTTCTCAGTGTGTCAATTTGACAGG |
| | | QLESKMSGKGQQQMRACMVTNNT | CGGCTCTGGAGGAGGCGGCTCCGGAGGCAGAATGGCTGGAAATGGAG |
| | | FTLKVPHVGEIPVAYRKVLLKTIQPR | GAGATGCTGCTCTGGCTCTGCTGCTGCTGGATAGACTGAATCAGCTGG |
| | | VEKYLFDESGEFKLSEVGPEHSLAE | AATCAAAAATGTCTGGAAAAGGACAGCAACAGATGAGAGCTTGCATG |
| | | YKMCGYKHWPQIAQFAPSASAFFG | GTGACAAACAACACATTTACACTGAAAGTGCCTCATGTGGGAGAAATT |
| | | MSRIGMEVTPSGTWLTYTGAIKLDD | CCTGTGGCTTACAGAAAAGTGCTGCTGAAAACAATTCAACCAAGAGTG |
| | | KDPNFKDQVILLNKHIDAYKTFPAR | GAAAAATACCTGTTTGATGAATCTGGAGAATTTAAACTTTCTGAAGTG |
| | | CATTDPSFLGRYMSALFADDLNQLT | GGACCTGAACACTCTCTTGCTGAATACAAAATGTGCGGATACAAACAC |
| | | GYHTDFSSEIIGYQLMCQPILLAEAE | TGGCCTCAGATTGCTCAGTTTGCTCCTTCTGCTTCTGCTTTCTTTGGAAT |
| | | LAKNVSLILGTVSWNLKKQGFAYA | GAGCAGAATTGGAATGGAAGTGACACCTTCTGGAACATGGCTGACATA |
| | | NRNRFLYIIKLIFLWLLWPVTLACFV | CACAGGAGCAATCAAACTGGATGATAAAGATCCAAACTTTAAAGATCA |
| | | LAAVYRINWITGGIAIAMACLVGLM | GGTGATTCTGCTGAACAAACACATTGATGCTTACAAAACATTTCCTGCT |
| | | WLSYFIASFRLFYRSYLLSAGIFGAIT | AGATGTGCTACAACTGATCCTTCTTTTCTGGGAAGATACATGTCTGCTC |
| | | DVFYKENSYKVPTDNYITTYARMA | TTTTTGCTGATGATTTAAATCAGTTAACAGGATATCACACAGATTTTTC |
| | | APKEIIFLEGETLFGDDTVIEVSMFNL | TTCTGAAATCATTGGATACCAGTTGATGTGTCAACCAATTTTGTTGGCT |
| | | GRCDIKDLPKEITVATSRTLSYYKLG | GAAGCTGAATTGGCAAAAAACGTGTCTTTGATTCTTGGAACAGTTTCTT |
| | | ASQRVAGGSGGGGSGGEQYIKWPW | GGAATCTTAAAAAACAAGGATTTGCATACGCAAATAGAAATAGATTTC |
| | | YIWLGFIAGLIAIVMVTIMLCCMTSC | TGTACATTATTAAACTGATTTTTCTGTGGCTGCTGTGGCCTGTGACACT |
| | | CSCLKGCCSCGSCCKFDEDDSEPVL | GGCTTGCTTTGTGCTGGCTGCTGTGTACAGAATAAATTGGATAACAGG |
| | | KGVKLHYT** (SEQ ID NO: RS | AGGAATTGCAATTGCAATGGCATGCTTGGTGGGATTGATGTGGTTGTC |
| | | C6p2full) | TTACTTTATTGCTTCTTTTAGACTGTTTTACAGATCCTACTTATTATCTG |
| | | | CTGGAATTTTTGGAGCTATCACAGATGTGTTTTACAAAGAAAACTCAT |
| | | | ACAAAGTGCCAACAGACAATTACATAACAACATACGCTAGAATGGCTG |
| | | | CTCCAAAAGAAATCATCTTCCTTGAAGGAGAAACACTTTTTGGAGATG |
| | | | ATACAGTGATTGAAGTGTCCATGTTTAATCTGGGAAGATGCGATATTA |
| | | | AAGATCTGCCAAAAGAAATTACAGTGGCAACAAGCAGAACACTGAGC |
| | | | TACTACAAACTGGGAGCAAGCCAGAGAGTGGCAGGAGGATCCGGTGG |
| | | | TGGCGGCAGCGGCGGCGAACAGTACATTAAATGGCCTTGGTACATTTG |
| | | | GCTTGGATTTATTGCAGGATTAATTGCAATTGTGATGGTGACAATTATG |
| | | | TTATGTTGTATGACATCATGTTGTTCTTGTTTAAAAGGATGTTGTTCTTG |
| | | | TGGAAGCTGTTGTAAATTTGATGAAGATGATTCTGAACCTGTGTTAAA |
| | | | AGGAGTGAAATTGCATTACACATGATGACTCGAGCTGGTACTGCATGC |
| | | | ACGCAATGCTAGCTGCCCCTTTCCCGTCCTGGGTACCCCGAGTCTCCCC |
| | | | CGACCTCGGGTCCCAGGTATGCTCCCACCTCCACCTGCCCCACTCACCA |
| | | | CCTCTGCTAGTTCCAGACACCTCCCAAGCACGCAGCAATGCAGCTCAA |
| | | | AACGCTTAGCCTAGCCACACCCCCACGGGAAACAGCAGTGATTAACCT |
| | | | TTAGCAATAAACGAAAGTTTAACTAAGCTATACTAACCCCAGGGTTGG |
| | | | TCAATTTCGTGCCAGCCACACCCTGGAGCTAGCAAAAAAAAAAAAAA |
| | | | AAAAAAAAAAAAAAAAGCATATGACTAAAAAAAAAAAAAAAAAAAA |
| | | | AAAAAAAAAAAAAAAAAAAAAAAAAAAAAAAAAAAAAAAAAAAAA |
| | | | AAAAA (SEQ ID NO: RS Con2full) |
| | | | ATGAGAGTGATGGCCCCCAGAACCCTGATCCTGCTGCTGTCTGGCGCC |
| | | | CTGGCCCTGACAGAGACATGGGCCGGAAGCGGCGGCTCTGGAGGAGG |
| | | | CGGCTCCGGAGGCAGAATGGCTGGAAATGGAGGAGATGCTGCTCTGG |
| | | | CTCTGCTGCTGCTGGATAGACTGAATCAGCTGGAATCAAAAATGTCTG |
| | | | GAAAAGGACAGCAACAGATGAGAGCTTGCATGGTGACAAACAACACA |
| | | | TTTACACTGAAAGTGCCTCATGTGGGAGAAATTCCTGTGGCTTACAGA |
| | | | AAAGTGCTGCTGAAAACAATTCAACCAAGAGTGGAAAAATACCTGTTT |
| | | | GATGAATCTGGAGAATTTAAACTTTCTGAAGTGGGACCTGAACACTCT |
| | | | CTTGCTGAATACAAAATGTGCGGATACAAACACTGGCCTCAGATTGCT |
| | | | CAGTTTGCTCCTTCTGCTTCTGCTTTCTTTGGAATGAGCAGAATTGGAA |
| | | | TGGAAGTGACACCTTCTGGAACATGGCTGACATACACAGGAGCAATCA |
| | | | AACTGGATGATAAAGATCCAAACTTTAAAGATCAGGTGATTCTGCTGA |
| | | | ACAAACACATTGATGCTTACAAAACATTTCCTGCTAGATGTGCTACAA |
| | | | CTGATCCTTCTTTTCTGGGAAGATACATGTCTGCTCTTTTTGCTGATGAT |
| | | | TTAAATCAGTTAACAGGATATCACACAGATTTTTCTTCTGAAATCATTG |
| | | | GATACCAGTTGATGTGTCAACCAATTTTGTTGGGTGAAGCAGAATTGG |
| | | | CAAAAAACGTGTCTTTGATTCTTGGAACAGTTTCTTGGAATCTTAAAAA |
| | | | ACAAGGATTTGCATACGCAAATAGAAATAGATTTCTGTACATTATTAA |
| | | | ACTGATTTTTCTGTGGCTGCTGTGGCCTGTGACACTGGCTTGCTTTGTG |
| | | | CTGGCTGCTGTGTACAGAATAAATTGGATAACAGGAGGAATTGCAATT |
| | | | GCAATGGCATGCTTGGTGGGATTGATGTGGTTGTCTTACTTTATTGCTT |
| | | | CTTTTAGACTGTTTTACAGATCCTACTTATTATCTGCTGGAATTTTTGGA |
| | | | GCTATCACAGATGTGTTTTACAAAGAAAACTCATACAAAGTGCCAACA |
| | | | GACAATTACATAACAACATACGCTAGAATGGCTGCTCCAAAAGAAATC |
| | | | ATCTTCCTTGAAGGAGAAACACTTTTTGGAGATGATACAGTGATTGAA |
| | | | GTGTCCATGTTTAATCTGGGAAGATGCGATATTAAAGATCTGCCAAAA |
| | | | GAAATTACAGTGGCAACAAGCAGAACACTGAGCTACTACAAACTGGG |
| | | | AGCAAGCCAGAGAGTGGCAGGTGGTTCTCTTGGCGGAGGGGGTTCGG |
| | | | GAATCGTGGGAATTGTGGCAGGACTGGCAGTGCTGGCCGTGGTGGTGA |
| | | | TCGGAGCCGTGGTGGCTACCGTGATGTGCAGACGGAAGTCCAGCGGAG |
| | | | GCAAGGGCGGCAGCTACAGCCAGGCCGCCAGCTCTGATAGCGCCCAG |
| | | | GGCAGCGACGTGTCACTGACAGCCTAGTAA (SEQ ID NO: RS C6n3) |
| RS-C7 | dEarI- | | ATTCTTCTGGTCCCCACAGACTCAGAGAGAACCC |
| | hAg | | |
| RS-C7 | Kozak | | GCCACC |
| RS-C7 | SEC | MFVFLVLLPLVSSQCVNLT | ATGTTTGTTTTTCTTGTTTTACTTCCTTTAGTCTCTTCTCAGTGTGTCAAT |
| | | | TTGACA |
| RS-C7 | RS-C7 | GGSGGGGSGGSPARMAGNGGDAAL | GGCGGCTCTGGAGGAGGCGGCTCCGGAGGCTCTCCTGCCAGAATGGCT |
| | | ALLLLDRLNQLESKMSGKGQQQQG | GGAAATGGAGGAGATGCTGCTCTGGCTCTGCTGCTGCTGGATAGACTG |
| | | QTVTKKSAAEASKKPRQKRTATKA | AATCAGCTGGAAAGCAAAATGAGCGGAAAAGGACAGCAGCAGCAGGG |
| | | YNVTQAFGRRGPEQTQGNFGDQELI | ACAAACAGTGACAAAGAAATCTGCTGCTGAAGCCAGCAAGAAACCAA |
| | | RQGTDYKHWPQIAQFAPSASAFFGM | GACAGAAAAGAACAGCCACAAAAGCCTACAATGTGACACAGGCCTTT |
| | | SRIGMEVTPSGTWLTYTGAIKLDDK | GGAAGAAGGGGACCTGAACAGACACAGGGAAATTTTGGAGATCAGGA |
| | | DPNFKDQVILLNKHIDAYKTFPPTEP | ACTGATCAGACAGGGAACAGATTACAAACACTGGCCTCAGATCGCCCA |
| | | KKDKFKAAMVTNNTFTLKVPHVGE | GTTTGCCCCATCTGCCTCTGCCTTCTTTGGAATGAGCAGAATTGGAATG |
| | | IPVAYRKVLLKTIQPRVEKYLFDESG | GAAGTGACACCTTCTGGAACATGGCTGACATACACAGGAGCCATCAAA |
| | | EFKLSEVGPEHSLAEYYIFFASFYYK | CTGGATGATAAAGATCCAAATTTTAAAGATCAGGTGATTCTGCTGAAT |
| | | RNGFAYANRNRFLYIIKLIFLWLLWP | AAACACATTGATGCCTACAAAACATTTCCACCAACAGAACCAAAGAAA |
| | | VTLACFVLAAVYRINWITGGIAIAM | GATAAATTCAAAGCTGCTATGGTGACAAACAACACATTTACACTGAAA |
| | | ACLVGLMWLSYFIASFRLFYNSFHT | GTGCCTCATGTGGGAGAAATTCCTGTGGCTTACAGAAAAGTGCTGCTG |
| | | TDPSFLGRYMSALFADDLNQLTGYH | AAAACAATTCAACCAAGAGTGGAAAAATACCTGTTTGATGAATCTGGA |
| | | TDFSSEIIGYQLMCQPILLAEAELAK | GAATTTAAACTTTCTGAAGTGGGACCTGAACACTCTCTTGCTGAATACT |
| | | NVSLILGTVSWNLKKNGLGRCDIKD | ACATTTTCTTTGCTTCTTTTTACTATAAGAGGAATGGATTTGCATACGC |
| | | LPKEITVATSRTLSYYKLGASQRVA | AAATAGAAATAGATTTCTGTACATTATTAAACTGATTTTTCTGTGGCTG |
| | | YRSYLLSAGIFGAITDVFYKENSYKV | CTGTGGCCTGTGACACTGGCTTGCTTTGTGCTGGCTGCTGTGTACAGAA |
| | | PTDNYITTYARMAAPKEIIFLEGETL | TAAATTGGATAACAGGAGGAATTGCAATTGCAATGGCATGCTTGGTGG |
| | | FGDDTVIEVAIILASFSASTRRRGGGS | GATTGATGTGGTTGTCTTACTTTATTGCTTCTTTTAGACTGTTTTACAAC |
| | | GGGGSGG (SEQ ID NO: RS C7p2) | TCTTTTCACACAACTGATCCATCTTTTCTGGGAAGATACATGTCTGCTC |
| | | | TTTTTGCTGATGATTTAAATCAGTTAACAGGATATCACACAGATTTTTC |
| | | | TTCTGAAATCATTGGATACCAGTTGATGTGTCAACCAATTTTGTTGGCT |
| | | | GAAGCTGAATTGGCAAAAAACGTGTCTTTGATTCTTGGAACAGTTTCTT |
| | | | GGAATCTTAAGAAAAACGGACTGGGAAGATGCGATATTAAAGATCTG |
| | | | CCAAAAGAAATTACAGTGGCAACAAGCAGAACACTGAGCTACTACAA |
| | | | ACTGGGAGCAAGCCAGAGAGTGGCATACAGATCCTACTTATTATCTGC |
| | | | TGGAATTTTTGGAGCTATCACAGATGTGTTTTACAAAGAAAACTCATA |
| | | | CAAAGTGCCAACAGACAATTACATAACAACATACGCTAGAATGGCTGC |
| | | | TCCAAAAGAAATCATCTTCCTTGAAGGAGAAACACTTTTTGGAGATGA |
| | | | TACAGTGATTGAAGTGGCAATTATTCTGGCATCTTTTTCTGCATCCACA |
| | | | AGAAGAAGGGGAGGAGGATCCGGTGGTGGCGGCAGCGGCGGC (SEQ |
| | | | ID NO: RS C7n2) |
| RS-C7 | TM | EQYIKWPWYIWLGFIAGLIAIVMVTI | GAACAGTACATTAAATGGCCTTGGTACATTTGGCTTGGATTTATTGCAG |
| | | MLCCMTSCCSCLKGCCSCGSCCKFD | GATTAATTGCAATTGTGATGGTGACAATTATGTTATGTTGTATGACATC |
| | | EDDSEPVLKGVKLHYT** | ATGTTGTTCTTGTTTAAAAGGATGTTGTTCTTGTGGAAGCTGTTGTAAA |
| | | | TTTGATGAAGATGATTCTGAACCTGTGTTAAAAGGAGTGAAATTGCAT |
| | | | TACACATGATGA |
| RS-C7 | | | CTCGAG |
| RS-C7 | F element | | CTGGTACTGCATGCACGCAATGCTAGCTGCCCCTTTCCCGTCCTGGGTA |
| | | | CCCCGAGTCTCCCCCGACCTCGGGTCCCAGGTATGCTCCCACCTCCACC |
| | | | TGCCCCACTCACCACCTCTGCTAGTTCCAGACACCTCC |
| RS-C7 | I element | | CAAGCACGCAGCAATGCAGCTCAAAACGCTTAGCCTAGCCACACCCCC |
| | | | ACGGGAAACAGCAGTGATTAACCTTTAGCAATAAACGAAAGTTTAACT |
| | | | AAGCTATACTAACCCCAGGGTTGGTCAATTTCGTGCCAGCCACACC |
| RS-C7 | | | CTGGAGCTAGC |
| RS-C7 | PolyA | | AAAAAAAAAAAAAAAAAAAAAAAAAAAAAAGCATATGACTAAAAAA |
| | | | AAAAAAAAAAAAAAAAAAAAAAAAAAAAAAAAAAAAAAAAAAAAA |
| | | | AAAAAAAAAAAAAAAAAAA |
| RS-C7 | FullSeq | MFVFLVLLPLVSSQCVNLTGGSGGG | ATTCTTCTGGTCCCCACAGACTCAGAGAGAACCCGCCACCATGTTTGTT |
| | | GSGGSPARMAGNGGDAALALLLLD | TTTCTTGTTTTACTTCCTTTAGTCTCTTCTCAGTGTGTCAATTTGACAGG |
| | | RLNQLESKMSGKGQQQQGQTVTKK | CGGCTCTGGAGGAGGCGGCTCCGGAGGCTCTCCTGCCAGAATGGCTGG |
| | | SAAEASKKPRQKRTATKAYNVTQA | AAATGGAGGAGATGCTGCTCTGGCTCTGCTGCTGCTGGATAGACTGAA |
| | | FGRRGPEQTQGNFGDQELIRQGTDY | TCAGCTGGAAAGCAAAATGAGCGGAAAAGGACAGCAGCAGCAGGGAC |
| | | KHWPQIAQFAPSASAFFGMSRIGME | AAACAGTGACAAAGAAATCTGCTGCTGAAGCCAGCAAGAAACCAAGA |
| | | VTPSGTWLTYTGAIKLDDKDPNFKD | CAGAAAAGAACAGCCACAAAAGCCTACAATGTGACACAGGCCTTTGG |
| | | QVILLNKHIDAYKTFPPTEPKKDKFK | AAGAAGGGGACCTGAACAGACACAGGGAAATTTTGGAGATCAGGAAC |
| | | AAMVTNNTFTLKVPHVGEIPVAYRK | TGATCAGACAGGGAACAGATTACAAACACTGGCCTCAGATCGCCCAGT |
| | | VLLKTIQPRVEKYLFDESGEFKLSEV | TTGCCCCATCTGCCTCTGCCTTCTTTGGAATGAGCAGAATTGGAATGGA |
| | | GPEHSLAEYYIFFASFYYKRNGFAY | AGTGACACCTTCTGGAACATGGCTGACATACACAGGAGCCATCAAACT |
| | | ANRNRFLYIIKLIFLWLLWPVTLACF | GGATGATAAAGATCCAAATTTTAAAGATCAGGTGATTCTGCTGAATAA |
| | | VLAAVYRINWITGGIAIAMACLVGL | ACACATTGATGCCTACAAAACATTTCCACCAACAGAACCAAAGAAAGA |
| | | MWLSYFIASFRLFYNSFHTTDPSFLG | TAAATTCAAAGCTGCTATGGTGACAAACAACACATTTACACTGAAAGT |
| | | RYMSALFADDLNQLTGYHTDESSEII | GCCTCATGTGGGAGAAATTCCTGTGGCTTACAGAAAAGTGCTGCTGAA |
| | | GYQLMCQPILLAEAELAKNVSLILG | AACAATTCAACCAAGAGTGGAAAAATACCTGTTTGATGAATCTGGAGA |
| | | TVSWNLKKNGLGRCDIKDLPKEITV | ATTTAAACTTTCTGAAGTGGGACCTGAACACTCTCTTGCTGAATACTAC |
| | | ATSRTLSYYKLGASQRVAYRSYLLS | ATTTTCTTTGCTTCTTTTTACTATAAGAGGAATGGATTTGCATACGCAA |
| | | AGIFGAITDVFYKENSYKVPTDNYIT | ATAGAAATAGATTTCTGTACATTATTAAACTGATTTTTCTGTGGCTGCT |
| | | TYARMAAPKEIIFLEGETLFGDDTVI | GTGGCCTGTGACACTGGCTTGCTTTGTGCTGGCTGCTGTGTACAGAATA |
| | | EVAIILASFSASTRRRGGGSGGGGSG | AATTGGATAACAGGAGGAATTGCAATTGCAATGGCATGCTTGGTGGGA |
| | | GEQYIKWPWYIWLGFIAGLIAIVMV | TTGATGTGGTTGTCTTACTTTATTGCTTCTTTTAGACTGTTTTACAACTC |
| | | TIMLCCMTSCCSCLKGCCSCGSCCK | TTTTCACACAACTGATCCATCTTTTCTGGGAAGATACATGTCTGCTCTT |
| | | FDEDDSEPVLKGVKLHYT** (SEQ | TTTGCTGATGATTTAAATCAGTTAACAGGATATCACACAGATTTTTCTT |
| | | ID NO: RS C7p2full) | CTGAAATCATTGGATACCAGTTGATGTGTCAACCAATTTTGTTGGCTGA |
| | | | AGCTGAATTGGCAAAAAACGTGTCTTTGATTCTTGGAACAGTTTCTTGG |
| | | | AATCTTAAGAAAAACGGACTGGGAAGATGCGATATTAAAGATCTGCCA |
| | | | AAAGAAATTACAGTGGCAACAAGCAGAACACTGAGCTACTACAAACT |
| | | | GGGAGCAAGCCAGAGAGTGGCATACAGATCCTACTTATTATCTGCTGG |
| | | | AATTTTTGGAGCTATCACAGATGTGTTTTACAAAGAAAACTCATACAA |
| | | | AGTGCCAACAGACAATTACATAACAACATACGCTAGAATGGCTGCTCC |
| | | | AAAAGAAATCATCTTCCTTGAAGGAGAAACACTTTTTGGAGATGATAC |
| | | | AGTGATTGAAGTGGCAATTATTCTGGCATCTTTTTCTGCATCCACAAGA |
| | | | AGAAGGGGAGGAGGATCCGGTGGTGGCGGCAGCGGCGGCGAACAGTA |
| | | | CATTAAATGGCCTTGGTACATTTGGCTTGGATTTATTGCAGGATTAATT |
| | | | GCAATTGTGATGGTGACAATTATGTTATGTTGTATGACATCATGTTGTT |
| | | | CTTGTTTAAAAGGATGTTGTTCTTGTGGAAGCTGTTGTAAATTTGATGA |
| | | | AGATGATTCTGAACCTGTGTTAAAAGGAGTGAAATTGCATTACACATG |
| | | | ATGACTCGAGCTGGTACTGCATGCACGCAATGCTAGCTGCCCCTTTCCC |
| | | | GTCCTGGGTACCCCGAGTCTCCCCCGACCTCGGGTCCCAGGTATGCTCC |
| | | | CACCTCCACCTGCCCCACTCACCACCTCTGCTAGTTCCAGACACCTCCC |
| | | | AAGCACGCAGCAATGCAGCTCAAAACGCTTAGCCTAGCCACACCCCCA |
| | | | CGGGAAACAGCAGTGATTAACCTTTAGCAATAAACGAAAGTTTAACTA |
| | | | AGCTATACTAACCCCAGGGTTGGTCAATTTCGTGCCAGCCACACCCTG |
| | | | GAGCTAGCAAAAAAAAAAAAAAAAAAAAAAAAAAAAAAGCATATGA |
| | | | CTAAAAAAAAAAAAAAAAAAAAAAAAAAAAAAAAAAAAAAAAAAAA |
| | | | AAAAAAAAAAAAAAAAAAAAAAAAAA (SEQ ID NO: RS C7n2full) |
| RS-C7 | SEC | MRVMAPRTLILLLSGALALTETWAG | ATGAGAGTGATGGCCCCCAGAACCCTGATCCTGCTGCTGTCTGGCGCC |
| | | S | CTGGCCCTGACAGAGACATGGGCCGGAAGC |
| RS-C7 | | | GGCGGCTCTGGAGGAGGCGGCTCCGGAGGCTCTCCTGCCAGAATGGCT |
| | | | GGAAATGGAGGAGATGCTGCTCTGGCTCTGCTGCTGCTGGATAGACTG |
| | | | AATCAGCTGGAAAGCAAAATGAGCGGAAAAGGACAGCAGCAGCAGGG |
| | | | ACAAACAGTGACAAAGAAATCTGCTGCTGAAGCCAGCAAGAAACCAA |
| | | | GACAGAAAAGAACAGCCACAAAAGCCTACAATGTGACACAGGCCTTT |
| | | | GGAAGAAGGGGACCTGAACAGACACAGGGAAATTTTGGAGATCAGGA |
| | | | ACTGATCAGACAGGGAACAGATTACAAACACTGGCCTCAGATCGCCCA |
| | | | GTTTGCCCCATCTGCCTCTGCCTTCTTTGGAATGAGCAGAATTGGAATG |
| | | | GAAGTGACACCTTCTGGAACATGGCTGACATACACAGGAGCCATCAAA |
| | | | CTGGATGATAAAGATCCAAATTTTAAAGATCAGGTGATTCTGCTGAAT |
| | | | AAACACATTGATGCCTACAAAACATTTCCACCAACAGAACCAAAGAAA |
| | | | GATAAATTCAAAGCTGCTATGGTGACAAACAACACATTTACACTGAAA |
| | | | GTGCCTCATGTGGGAGAAATTCCTGTGGCTTACAGAAAAGTGCTGCTG |
| | | | AAAACAATTCAACCAAGAGTGGAAAAATACCTGTTTGATGAATCTGGA |
| | | | GAATTTAAACTTTCTGAAGTGGGACCTGAACACTCTCTTGCTGAATACT |
| | | | ACATTTTCTTTGCTTCTTTTTACTATAAGAGGAATGGATTTGCATACGC |
| | | | AAATAGAAATAGATTTCTGTACATTATTAAACTGATTTTTCTGTGGCTG |
| | | | CTGTGGCCTGTGACACTGGCTTGCTTTGTGCTGGCTGCTGTGTACAGAA |
| | | | TAAATTGGATAACAGGAGGAATTGCAATTGCAATGGCATGCTTGGTGG |
| | | | GATTGATGTGGTTGTCTTACTTTATTGCTTCTTTTAGACTGTTTTACAAC |
| | | | TCTTTTCACACAACTGATCCATCTTTTCTGGGAAGATACATGTCTGCTC |
| | | | TTTTTGCTGATGATTTAAATCAGTTAACAGGATATCACACAGATTTTTC |
| | | | TTCTGAAATCATTGGATACCAGTTGATGTGTCAACCAATTTTGTTGGGT |
| | | | GAAGCAGAATTGGCAAAAAACGTGTCTTTGATTCTTGGAACAGTTTCT |
| | | | TGGAATCTTAAGAAAAACGGACTGGGAAGATGCGATATTAAAGATCTG |
| | | | CCAAAAGAAATTACAGTGGCAACAAGCAGAACACTGAGCTACTACAA |
| | | | ACTGGGAGCAAGCCAGAGAGTGGCATACAGATCCTACTTATTATCTGC |
| | | | TGGAATTTTTGGAGCTATCACAGATGTGTTTTACAAAGAAAACTCATA |
| | | | CAAAGTGCCAACAGACAATTACATAACAACATACGCTAGAATGGCTGC |
| | | | TCCAAAAGAAATCATCTTCCTTGAAGGAGAAACACTTTTTGGAGATGA |
| | | | TACAGTGATTGAAGTGGCAATTATTCTGGCATCTTTTTCTGCATCCACA |
| | | | AGAAGAAGGGGAGGTGGTTCTCTTGGCGGAGGGGGTTCGGGA (SEQ ID |
| | | | NO: RS C7n3) |
| RS-C7 | TM | | ATCGTGGGAATTGTGGCAGGACTGGCAGTGCTGGCCGTGGTGGTGATC |
| | | | GGAGCCGTGGTGGCTACCGTGATGTGCAGACGGAAGTCCAGCGGAGG |
| | | | CAAGGGCGGCAGCTACAGCCAGGCCGCCAGCTCTGATAGCGCCCAGG |
| | | | GCAGCGACGTGTCACTGACAGCCTAGTAA |
| RS-C7 | | | AGAATAAACTAGT |
| RS-C7 | dEarI- | | ATTCTTCTGGTCCCCACAGACTCAGAGAGAACCC |
| | hAg | | |
| RS-C7 | Kozak | | GCCACC |
| RS-C7 | SEC | MFVFLVLLPLVSSQCVNLT | ATGTTTGTTTTTCTTGTTTTACTTCCTTTAGTCTCATCTCAGTGTGTCAA |
| | | | TTTGACA |
| RS-C7 | RS-C7 | GGSGGGGSGGSPARMAGNGGDAAL | GGCGGCTCTGGAGGAGGCGGCTCCGGAGGCTCTCCTGCCAGAATGGCT |
| | | ALLLLDRLNQLESKMSGKGQQQQG | GGAAATGGAGGAGATGCTGCTCTGGCTCTGCTGCTGCTGGATAGACTG |
| | | QTVTKKSAAEASKKPRQKRTATKA | AATCAGCTGGAAAGCAAAATGAGCGGAAAAGGACAGCAGCAGCAGGG |
| | | YNVTQAFGRRGPEQTQGNFGDQELI | ACAAACAGTGACAAAGAAATCTGCTGCTGAAGCCAGCAAGAAACCAA |
| | | RQGTDYKHWPQIAQFAPSASAFFGM | GACAGAAAAGAACAGCCACAAAAGCCTACAATGTGACACAGGCCTTT |
| | | SRIGMEVTPSGTWLTYTGAIKLDDK | GGAAGAAGGGGACCTGAACAGACACAGGGAAATTTTGGAGATCAGGA |
| | | DPNFKDQVILLNKHIDAYKTFPPTEP | ACTGATCAGACAGGGAACAGATTACAAACACTGGCCTCAGATCGCCCA |
| | | KKDKFKAAMVTNNTFTLKVPHVGE | GTTTGCCCCATCTGCCTCTGCCTTCTTTGGAATGAGCAGAATTGGAATG |
| | | IPVAYRKVLLKTIQPRVEKYLFDESG | GAAGTGACACCTTCTGGAACATGGCTGACATACACAGGAGCCATCAAA |
| | | EFKLSEVGPEHSLAEYYIFFASFYYK | CTGGATGATAAAGATCCAAATTTTAAAGATCAGGTGATTCTGCTGAAT |
| | | RNGFAYANRNRFLYIIKLIFLWLLWP | AAACACATTGATGCCTACAAAACATTTCCACCAACAGAACCAAAGAAA |
| | | VTLACFVLAAVYRINWITGGIAIAM | GATAAATTCAAAGCTGCTATGGTGACAAACAACACATTTACACTGAAA |
| | | ACLVGLMWLSYFIASFRLFYNSFHT | GTGCCTCATGTGGGAGAAATTCCTGTGGCTTACAGAAAAGTGCTGCTG |
| | | TDPSFLGRYMSALFADDLNQLTGYH | AAAACAATTCAACCAAGAGTGGAAAAATACCTGTTTGATGAATCTGGA |
| | | TDFSSEIIGYQLMCQPILLAEAELAK | GAATTTAAACTTTCTGAAGTGGGACCTGAACACTCTCTTGCTGAATACT |
| | | NVSLILGTVSWNLKKNGLGRCDIKD | ACATTTTCTTTGCTTCTTTTTACTATAAGAGGAATGGATTTGCATACGC |
| | | LPKEITVATSRTLSYYKLGASQRVA | AAATAGAAATAGATTTCTGTACATTATTAAACTGATTTTTCTGTGGCTG |
| | | YRSYLLSAGIFGAITDVFYKENSYKV | CTGTGGCCTGTGACACTGGCTTGCTTTGTGCTGGCTGCTGTGTACAGAA |
| | | PTDNYITTYARMAAPKEIIFLEGETL | TAAATTGGATAACAGGAGGAATTGCAATTGCAATGGCATGCTTGGTGG |
| | | FGDDTVIEVAIILASFSASTRRRGGGS | GATTGATGTGGTTGTCTTACTTTATTGCTTCTTTTAGACTGTTTTACAAC |
| | | GGGGSGG (SEQ ID NO: RS C7p4) | TCTTTTCACACAACTGATCCATCTTTTCTGGGAAGATACATGTCTGCTC |
| | | | TTTTTGCTGATGATTTAAATCAGTTAACAGGATATCACACAGATTTTTC |
| | | | TTCTGAAATCATTGGATACCAGTTGATGTGTCAACCAATTTTGTTGGCT |
| | | | GAAGCTGAATTGGCAAAAAACGTGTCTTTGATTCTTGGAACAGTTTCTT |
| | | | GGAATCTTAAGAAAAACGGACTGGGAAGATGCGATATTAAAGATCTG |
| | | | CCAAAAGAAATTACAGTGGCAACAAGCAGAACACTGAGCTACTACAA |
| | | | ACTGGGAGCAAGCCAGAGAGTGGCATACAGATCCTACTTATTATCTGC |
| | | | TGGAATTTTTGGAGCTATCACAGATGTGTTTTACAAAGAAAACTCATA |
| | | | CAAAGTGCCAACAGACAATTACATAACAACATACGCTAGAATGGCTGC |
| | | | TCCAAAAGAAATCATCTTCCTTGAAGGAGAAACACTTTTTGGAGATGA |
| | | | TACAGTGATTGAAGTGGCAATTATTCTGGCATCTTTTTCTGCATCCACA |
| | | | AGAAGAAGGGGAGGAGGATCCGGTGGTGGCGGCAGCGGCGGC (SEQ |
| | | | ID NO: RS C7n4) |
| RS-C7 | TM | EQYIKWPWYIWLGFIAGLIAIVMVTI | GAACAGTACATTAAATGGCCTTGGTACATTTGGCTTGGATTTATTGCAG |
| | | MLCCMTSCCSCLKGCCSCGSCCKFD | GATTAATTGCAATTGTGATGGTGACAATTATGTTATGTTGTATGACATC |
| | | EDDSEPVLKGVKLHYT** | ATGTTGTTCTTGTTTAAAAGGATGTTGTTCTTGTGGAAGCTGTTGTAAA |
| | | | TTTGATGAAGATGATTCTGAACCTGTGTTAAAAGGAGTGAAATTGCAT |
| | | | TACACATGATGA |
| RS-C7 | | | CTCGAG |
| RS-C7 | F element | | CTGGTACTGCATGCACGCAATGCTAGCTGCCCCTTTCCCGTCCTGGGTA |
| | | | CCCCGAGTCTCCCCCGACCTCGGGTCCCAGGTATGCTCCCACCTCCACC |
| | | | TGCCCCACTCACCACCTCTGCTAGTTCCAGACACCTCC |
| RS-C7 | I element | | CAAGCACGCAGCAATGCAGCTCAAAACGCTTAGCCTAGCCACACCCCC |
| | | | ACGGGAAACAGCAGTGATTAACCTTTAGCAATAAACGAAAGTTTAACT |
| | | | AAGCTATACTAACCCCAGGGTTGGTCAATTTCGTGCCAGCCACACC |
| RS-C7 | | | CTGGAGCTAGC |
| RS-C7 | PolyA | | AAAAAAAAAAAAAAAAAAAAAAAAAAAAAAGCATATGACTAAAAAA |
| | | | AAAAAAAAAAAAAAAAAAAAAAAAAAAAAAAAAAAAAAAAAAAAA |
| | | | AAAAAAAAAAAAAAAAAAA |
| RS-C7 | FullSeq | MFVFLVLLPLVSSQCVNLTGGSGGG | AGAATAAACTAGTATTCTTCTGGTCCCCACAGACTCAGAGAGAACCCG |
| | | GSGGSPARMAGNGGDAALALLLLD | CCACCATGTTTGTTTTTCTTGTTTTACTTCCTTTAGTCTCATCTCAGTGT |
| | | RLNQLESKMSGKGQQQQGQTVTKK | GTCAATTTGACAGGCGGCTCTGGAGGAGGCGGCTCCGGAGGCTCTCCT |
| | | SAAEASKKPRQKRTATKAYNVTQA | GCCAGAATGGCTGGAAATGGAGGAGATGCTGCTCTGGCTCTGCTGCTG |
| | | FGRRGPEQTQGNFGDQELIRQGTDY | CTGGATAGACTGAATCAGCTGGAAAGCAAAATGAGCGGAAAAGGACA |
| | | KHWPQIAQFAPSASAFFGMSRIGME | GCAGCAGCAGGGACAAACAGTGACAAAGAAATCTGCTGCTGAAGCCA |
| | | VTPSGTWLTYTGAIKLDDKDPNFKD | GCAAGAAACCAAGACAGAAAAGAACAGCCACAAAAGCCTACAATGTG |
| | | QVILLNKHIDAYKTFPPTEPKKDKFK | ACACAGGCCTTTGGAAGAAGGGGACCTGAACAGACACAGGGAAATTT |
| | | AAMVTNNTFTLKVPHVGEIPVAYRK | TGGAGATCAGGAACTGATCAGACAGGGAACAGATTACAAACACTGGC |
| | | VLLKTIQPRVEKYLFDESGEFKLSEV | CTCAGATCGCCCAGTTTGCCCCATCTGCCTCTGCCTTCTTTGGAATGAG |
| | | GPEHSLAEYYIFFASFYYKRNGFAY | CAGAATTGGAATGGAAGTGACACCTTCTGGAACATGGCTGACATACAC |
| | | ANRNRFLYIIKLIFLWLLWPVTLACF | AGGAGCCATCAAACTGGATGATAAAGATCCAAATTTTAAAGATCAGGT |
| | | VLAAVYRINWITGGIAIAMACLVGL | GATTCTGCTGAATAAACACATTGATGCCTACAAAACATTTCCACCAAC |
| | | MWLSYFIASFRLFYNSFHTTDPSFLG | AGAACCAAAGAAAGATAAATTCAAAGCTGCTATGGTGACAAACAACA |
| | | RYMSALFADDLNQLTGYHTDFSSEII | CATTTACACTGAAAGTGCCTCATGTGGGAGAAATTCCTGTGGCTTACA |
| | | GYQLMCQPILLAEAELAKNVSLILG | GAAAAGTGCTGCTGAAAACAATTCAACCAAGAGTGGAAAAATACCTG |
| | | TVSWNLKKNGLGRCDIKDLPKEITV | TTTGATGAATCTGGAGAATTTAAACTTTCTGAAGTGGGACCTGAACAC |
| | | ATSRTLSYYKLGASQRVAYRSYLLS | TCTCTTGCTGAATACTACATTTTCTTTGCTTCTTTTTACTATAAGAGGAA |
| | | AGIFGAITDVFYKENSYKVPTDNYIT | TGGATTTGCATACGCAAATAGAAATAGATTTCTGTACATTATTAAACTG |
| | | TYARMAAPKEIIFLEGETLFGDDTVI | ATTTTTCTGTGGCTGCTGTGGCCTGTGACACTGGCTTGCTTTGTGCTGG |
| | | EVAIILASFSASTRRRGGGSGGGGSG | CTGCTGTGTACAGAATAAATTGGATAACAGGAGGAATTGCAATTGCAA |
| | | GEQYIKWPWYIWLGFIAGLIAIVMV | TGGCATGCTTGGTGGGATTGATGTGGTTGTCTTACTTTATTGCTTCTTTT |
| | | TIMLCCMTSCCSCLKGCCSCGSCCK | AGACTGTTTTACAACTCTTTTCACACAACTGATCCATCTTTTCTGGGAA |
| | | FDEDDSEPVLKGVKLHYT** (SEQ | GATACATGTCTGCTCTTTTTGCTGATGATTTAAATCAGTTAACAGGATA |
| | | ID NO: RS C7p4full) | TCACACAGATTTTTCTTCTGAAATCATTGGATACCAGTTGATGTGTCAA |
| | | | CCAATTTTGTTGGCTGAAGCTGAATTGGCAAAAAACGTGTCTTTGATTC |
| | | | TTGGAACAGTTTCTTGGAATCTTAAGAAAAACGGACTGGGAAGATGCG |
| | | | ATATTAAAGATCTGCCAAAAGAAATTACAGTGGCAACAAGCAGAACA |
| | | | CTGAGCTACTACAAACTGGGAGCAAGCCAGAGAGTGGCATACAGATC |
| | | | CTACTTATTATCTGCTGGAATTTTTGGAGCTATCACAGATGTGTTTTAC |
| | | | AAAGAAAACTCATACAAAGTGCCAACAGACAATTACATAACAACATA |
| | | | CGCTAGAATGGCTGCTCCAAAAGAAATCATCTTCCTTGAAGGAGAAAC |
| | | | ACTTTTTGGAGATGATACAGTGATTGAAGTGGCAATTATTCTGGCATCT |
| | | | TTTTCTGCATCCACAAGAAGAAGGGGAGGAGGATCCGGTGGTGGCGGC |
| | | | AGCGGCGGCGAACAGTACATTAAATGGCCTTGGTACATTTGGCTTGGA |
| | | | TTTATTGCAGGATTAATTGCAATTGTGATGGTGACAATTATGTTATGTT |
| | | | GTATGACATCATGTTGTTCTTGTTTAAAAGGATGTTGTTCTTGTGGAAG |
| | | | CTGTTGTAAATTTGATGAAGATGATTCTGAACCTGTGTTAAAAGGAGT |
| | | | GAAATTGCATTACACATGATGACTCGAGCTGGTACTGCATGCACGCAA |
| | | | TGCTAGCTGCCCCTTTCCCGTCCTGGGTACCCCGAGTCTCCCCCGACCT |
| | | | CGGGTCCCAGGTATGCTCCCACCTCCACCTGCCCCACTCACCACCTCTG |
| | | | CTAGTTCCAGACACCTCCCAAGCACGCAGCAATGCAGCTCAAAACGCT |
| | | | TAGCCTAGCCACACCCCCACGGGAAACAGCAGTGATTAACCTTTAGCA |
| | | | ATAAACGAAAGTTTAACTAAGCTATACTAACCCCAGGGTTGGTCAATT |
| | | | TCGTGCCAGCCACACCCTGGAGCTAGCAAAAAAAAAAAAAAAAAAAA |
| | | | AAAAAAAAAAGCATATGACTAAAAAAAAAAAAAAAAAAAAAAAAAA |
| | | | AAAAAAAAAAAAAAAAAAAAAAAAAAAAAAAAAAAAAAAAAAAA |
| | | | (SEQ ID NO: RS C7n4full) |
| RS-C8 | dEarI- | | ATTCTTCTGGTCCCCACAGACTCAGAGAGAACCC |
| | hAg | | |
| RS-C8 | Kozak | | GCCACC |
| RS-C8 | SEC | MFVFLVLLPLVSSQCVNLT | ATGTTTGTTTTTCTTGTTTTACTTCCTTTAGTCTCTTCTCAGTGTGTCAAT |
| | | | TTGACA |
| RS-C8 | RS-C8 | GGSGGGGSGGSPARMAGNGGDAAL | GGCGGCTCTGGAGGAGGCGGCTCCGGAGGCTCTCCTGCCAGAATGGCT |
| | | ALLLLDRLNQLESKMSGKGQQQQG | GGAAATGGAGGAGATGCTGCTCTGGCTCTGCTGCTGCTGGATAGACTG |
| | | QTVTKKSAAEASKKPRQKRTATKA | AATCAGCTGGAAAGCAAAATGAGCGGAAAAGGACAGCAGCAGCAGGG |
| | | YNVTQAFGRRGPEQTQGNFGDQELI | ACAAACAGTGACAAAGAAATCTGCTGCTGAAGCCAGCAAGAAACCAA |
| | | RQGTDYKHWPQIAQFAPSASAFFGM | GACAGAAAAGAACAGCCACAAAAGCCTACAATGTGACACAGGCCTTT |
| | | SRIGMEVTPSGTWLTYTGAIKLDDK | GGAAGAAGGGGACCTGAACAGACACAGGGAAATTTTGGAGATCAGGA |
| | | DPNFKDQVILLNKHIDAYKTFPPTEP | ACTGATCAGACAGGGAACAGATTACAAACACTGGCCTCAGATCGCCCA |
| | | KKDKFRACMVTNNTFTLKVPHVGEI | GTTTGCCCCATCTGCCTCTGCCTTCTTTGGAATGAGCAGAATTGGAATG |
| | | PVAYRKVLLKTIQPRVEKYLFDESG | GAAGTGACACCTTCTGGAACATGGCTGACATACACAGGAGCCATCAAA |
| | | EFKLSEVGPEHSLAEYYIFFASFYYK | CTGGATGATAAAGATCCAAATTTTAAAGATCAGGTGATTCTGCTGAAT |
| | | RCFHTTDPSFLGRYMSALFADDLNQ | AAACACATTGATGCCTACAAAACATTTCCACCAACAGAACCAAAGAAA |
| | | LTGYHTDFSSEIIGYQLMCQPILLAE | GATAAATTTAGAGCTTGCATGGTGACAAACAACACATTTACACTGAAA |
| | | AELAKNVSLILGTVSWNLKKRYLLS | GTGCCTCATGTGGGAGAAATTCCTGTGGCTTACAGAAAAGTGCTGCTG |
| | | AGIFGAITDVFYKENSYKVPTDNYIT | AAAACAATTCAACCAAGAGTGGAAAAATACCTGTTTGATGAATCTGGA |
| | | TYARMAAPKEIIFLEGETLFGDDTVI | GAATTTAAACTTTCTGAAGTGGGACCTGAACACTCTCTTGCTGAATACT |
| | | EVAIILASFSASTRRRGKLLEQWNLV | ACATTTTCTTTGCTTCTTTTTACTATAAAAGATGTTTTCACACAACTGAT |
| | | IGFNRNRFLYIIKLIFLWLLWPVTLA | CCATCTTTTCTGGGAAGATACATGTCTGCTCTTTTTGCTGATGATTTAA |
| | | CFVLAAVYSELVIGAVILRGHLRIAG | ATCAGTTAACAGGATATCACACAGATTTTTCTTCTGAAATCATTGGATA |
| | | HHLGRTVATSRTLSYYKLGASQRVK | CCAGTTGATGTGTCAACCAATTTTGTTGGCTGAAGCTGAATTGGCAAA |
| | | RYSGLMWLSYFARYAGGSGGGGSG | AAACGTGTCTTTGATTCTTGGAACAGTTTCTTGGAATCTTAAAAAGAGA |
| | | G (SEQ ID NO: RS C8p2) | TACTTATTATCTGCTGGAATTTTTGGAGCTATCACAGATGTGTTTTACA |
| | | | AAGAAAACTCATACAAAGTGCCAACAGACAATTACATAACAACATAC |
| | | | GCCAGAATGGCTGCTCCAAAAGAAATCATCTTCCTTGAAGGAGAAACA |
| | | | CTTTTTGGAGATGATACAGTGATTGAAGTGGCAATTATTCTGGCATCTT |
| | | | TTTCTGCATCCACAAGAAGAAGGGGAAAACTGCTGGAACAGTGGAATC |
| | | | TTGTGATTGGATTTAATAGAAATAGATTTCTTTACATTATTAAATTGAT |
| | | | TTTTCTTTGGCTTCTTTGGCCTGTGACACTTGCTTGCTTTGTGCTTGCTG |
| | | | CTGTGTACTCTGAACTGGTGATTGGAGCTGTGATTCTGAGAGGACACC |
| | | | TGAGAATTGCTGGACACCACCTGGGAAGAACAGTGGCAACATCAAGA |
| | | | ACACTTTCTTACTACAAACTGGGAGCTTCTCAGAGAGTGAAAAGATAC |
| | | | TCTGGCCTGATGTGGCTGAGCTACTTTGCTAGATATGCTGGAGGATCCG |
| | | | GTGGTGGCGGCAGCGGCGGC (SEQ ID NO: RS C8n2) |
| RS-C8 | TM | EQYIKWPWYIWLGFIAGLIAIVMVTI | GAACAGTACATTAAATGGCCTTGGTACATTTGGCTTGGATTTATTGCAG |
| | | MLCCMTSCCSCLKGCCSCGSCCKFD | GATTAATTGCAATTGTGATGGTGACAATTATGTTATGTTGTATGACATC |
| | | EDDSEPVLKGVKLHYT** | ATGTTGTTCTTGTTTAAAAGGATGTTGTTCTTGTGGAAGCTGTTGTAAA |
| | | | TTTGATGAAGATGATTCTGAACCTGTGTTAAAAGGAGTGAAATTGCAT |
| | | | TACACATGATGA |
| RS-C8 | | | CTCGAG |
| RS-C8 | F element | | CTGGTACTGCATGCACGCAATGCTAGCTGCCCCTTTCCCGTCCTGGGTA |
| | | | CCCCGAGTCTCCCCCGACCTCGGGTCCCAGGTATGCTCCCACCTCCACC |
| | | | TGCCCCACTCACCACCTCTGCTAGTTCCAGACACCTCC |
| RS-C8 | I element | | CAAGCACGCAGCAATGCAGCTCAAAACGCTTAGCCTAGCCACACCCCC |
| | | | ACGGGAAACAGCAGTGATTAACCTTTAGCAATAAACGAAAGTTTAACT |
| | | | AAGCTATACTAACCCCAGGGTTGGTCAATTTCGTGCCAGCCACACC |
| RS-C8 | | | CTGGAGCTAGC |
| RS-C8 | PolyA | | AAAAAAAAAAAAAAAAAAAAAAAAAAAAAAGCATATGACTAAAAAA |
| | | | AAAAAAAAAAAAAAAAAAAAAAAAAAAAAAAAAAAAAAAAAAAAA |
| | | | AAAAAAAAAAAAAAAAAAA |
| RS-C8 | FullSeq | MFVFLVLLPLVSSQCVNLTGGSGGG | ATTCTTCTGGTCCCCACAGACTCAGAGAGAACCCGCCACCATGTTTGTT |
| | | GSGGSPARMAGNGGDAALALLLLD | TTTCTTGTTTTACTTCCTTTAGTCTCTTCTCAGTGTGTCAATTTGACAGG |
| | | RLNQLESKMSGKGQQQQGQTVTKK | CGGCTCTGGAGGAGGCGGCTCCGGAGGCTCTCCTGCCAGAATGGCTGG |
| | | SAAEASKKPRQKRTATKAYNVTQA | AAATGGAGGAGATGCTGCTCTGGCTCTGCTGCTGCTGGATAGACTGAA |
| | | FGRRGPEQTQGNFGDQELIRQGTDY | TCAGCTGGAAAGCAAAATGAGCGGAAAAGGACAGCAGCAGCAGGGAC |
| | | KHWPQIAQFAPSASAFFGMSRIGME | AAACAGTGACAAAGAAATCTGCTGCTGAAGCCAGCAAGAAACCAAGA |
| | | VTPSGTWLTYTGAIKLDDKDPNFKD | CAGAAAAGAACAGCCACAAAAGCCTACAATGTGACACAGGCCTTTGG |
| | | QVILLNKHIDAYKTFPPTEPKKDKFR | AAGAAGGGGACCTGAACAGACACAGGGAAATTTTGGAGATCAGGAAC |
| | | ACMVTNNTFTLKVPHVGEIPVAYRK | TGATCAGACAGGGAACAGATTACAAACACTGGCCTCAGATCGCCCAGT |
| | | VLLKTIQPRVEKYLFDESGEFKLSEV | TTGCCCCATCTGCCTCTGCCTTCTTTGGAATGAGCAGAATTGGAATGGA |
| | | GPEHSLAEYYIFFASFYYKRCFHTTD | AGTGACACCTTCTGGAACATGGCTGACATACACAGGAGCCATCAAACT |
| | | PSFLGRYMSALFADDLNQLTGYHTD | GGATGATAAAGATCCAAATTTTAAAGATCAGGTGATTCTGCTGAATAA |
| | | FSSEIIGYQLMCQPILLAEAELAKNV | ACACATTGATGCCTACAAAACATTTCCACCAACAGAACCAAAGAAAGA |
| | | SLILGTVSWNLKKRYLLSAGIFGAIT | TAAATTTAGAGCTTGCATGGTGACAAACAACACATTTACACTGAAAGT |
| | | DVFYKENSYKVPTDNYITTYARMA | GCCTCATGTGGGAGAAATTCCTGTGGCTTACAGAAAAGTGCTGCTGAA |
| | | APKEIIFLEGETLFGDDTVIEVAIILAS | AACAATTCAACCAAGAGTGGAAAAATACCTGTTTGATGAATCTGGAGA |
| | | FSASTRRRGKLLEQWNLVIGFNRNR | ATTTAAACTTTCTGAAGTGGGACCTGAACACTCTCTTGCTGAATACTAC |
| | | FLYIIKLIFLWLLWPVTLACFVLAAV | ATTTTCTTTGCTTCTTTTTACTATAAAAGATGTTTTCACACAACTGATCC |
| | | YSELVIGAVILRGHLRIAGHHLGRTV | ATCTTTTCTGGGAAGATACATGTCTGCTCTTTTTGCTGATGATTTAAAT |
| | | ATSRTLSYYKLGASQRVKRYSGLM | CAGTTAACAGGATATCACACAGATTTTTCTTCTGAAATCATTGGATACC |
| | | WLSYFARYAGGSGGGGSGGEQYIK | AGTTGATGTGTCAACCAATTTTGTTGGCTGAAGCTGAATTGGCAAAAA |
| | | WPWYIWLGFIAGLIAIVMVTIMLCC | ACGTGTCTTTGATTCTTGGAACAGTTTCTTGGAATCTTAAAAAGAGATA |
| | | MTSCCSCLKGCCSCGSCCKFDEDDS | CTTATTATCTGCTGGAATTTTTGGAGCTATCACAGATGTGTTTTACAAA |
| | | EPVLKGVKLHYT** (SEQ ID NO: RS | GAAAACTCATACAAAGTGCCAACAGACAATTACATAACAACATACGCC |
| | | C8p2full) | AGAATGGCTGCTCCAAAAGAAATCATCTTCCTTGAAGGAGAAACACTT |
| | | | TTTGGAGATGATACAGTGATTGAAGTGGCAATTATTCTGGCATCTTTTT |
| | | | CTGCATCCACAAGAAGAAGGGGAAAACTGCTGGAACAGTGGAATCTT |
| | | | GTGATTGGATTTAATAGAAATAGATTTCTTTACATTATTAAATTGATTT |
| | | | TTCTTTGGCTTCTTTGGCCTGTGACACTTGCTTGCTTTGTGCTTGCTGCT |
| | | | GTGTACTCTGAACTGGTGATTGGAGCTGTGATTCTGAGAGGACACCTG |
| | | | AGAATTGCTGGACACCACCTGGGAAGAACAGTGGCAACATCAAGAAC |
| | | | ACTTTCTTACTACAAACTGGGAGCTTCTCAGAGAGTGAAAAGATACTC |
| | | | TGGCCTGATGTGGCTGAGCTACTTTGCTAGATATGCTGGAGGATCCGG |
| | | | TGGTGGCGGCAGCGGCGGCGAACAGTACATTAAATGGCCTTGGTACAT |
| | | | TTGGCTTGGATTTATTGCAGGATTAATTGCAATTGTGATGGTGACAATT |
| | | | ATGTTATGTTGTATGACATCATGTTGTTCTTGTTTAAAAGGATGTTGTT |
| | | | CTTGTGGAAGCTGTTGTAAATTTGATGAAGATGATTCTGAACCTGTGTT |
| | | | AAAAGGAGTGAAATTGCATTACACATGATGACTCGAGCTGGTACTGCA |
| | | | TGCACGCAATGCTAGCTGCCCCTTTCCCGTCCTGGGTACCCCGAGTCTC |
| | | | CCCCGACCTCGGGTCCCAGGTATGCTCCCACCTCCACCTGCCCCACTCA |
| | | | CCACCTCTGCTAGTTCCAGACACCTCCCAAGCACGCAGCAATGCAGCT |
| | | | CAAAACGCTTAGCCTAGCCACACCCCCACGGGAAACAGCAGTGATTAA |
| | | | CCTTTAGCAATAAACGAAAGTTTAACTAAGCTATACTAACCCCAGGGT |
| | | | TGGTCAATTTCGTGCCAGCCACACCCTGGAGCTAGCAAAAAAAAAAAA |
| | | | AAAAAAAAAAAAAAAAAAGCATATGACTAAAAAAAAAAAAAAAAAA |
| | | | AAAAAAAAAAAAAAAAAAAAAAAAAAAAAAAAAAAAAAAAAAAAA |
| | | | AAAAAAA (SEQ ID NO: RS C8n2full) |
| | | | ATGAGAGTGATGGCCCCCAGAACCCTGATCCTGCTGCTGTCTGGCGCC |
| | | | CTGGCCCTGACAGAGACATGGGCCGGAAGCGGCGGCTCTGGAGGAGG |
| | | | CGGCTCCGGAGGCTCTCCTGCCAGAATGGCTGGAAATGGAGGAGATGC |
| | | | TGCTCTGGCTCTGCTGCTGCTGGATAGACTGAATCAGCTGGAAAGCAA |
| | | | AATGAGCGGAAAAGGACAGCAGCAGCAGGGACAAACAGTGACAAAG |
| | | | AAATCTGCTGCTGAAGCCAGCAAGAAACCAAGACAGAAAAGAACAGC |
| | | | CACAAAAGCCTACAATGTGACACAGGCCTTTGGAAGAAGGGGACCTG |
| | | | AACAGACACAGGGAAATTTTGGAGATCAGGAACTGATCAGACAGGGA |
| | | | ACAGATTACAAACACTGGCCTCAGATCGCCCAGTTTGCCCCATCTGCCT |
| | | | CTGCCTTCTTTGGAATGAGCAGAATTGGAATGGAAGTGACACCTTCTG |
| | | | GAACATGGCTGACATACACAGGAGCCATCAAACTGGATGATAAAGAT |
| | | | CCAAATTTTAAAGATCAGGTGATTCTGCTGAATAAACACATTGATGCC |
| | | | TACAAAACATTTCCACCAACAGAACCAAAGAAAGATAAATTTAGAGCT |
| | | | TGCATGGTGACAAACAACACATTTACACTGAAAGTGCCTCATGTGGGA |
| | | | GAAATTCCTGTGGCTTACAGAAAAGTGCTGCTGAAAACAATTCAACCA |
| | | | AGAGTGGAAAAATACCTGTTTGATGAATCTGGAGAATTTAAACTTTCT |
| | | | GAAGTGGGACCTGAACACTCTCTTGCTGAATACTACATTTTCTTTGCTT |
| | | | CTTTTTACTATAAAAGATGTTTTCACACAACTGATCCATCTTTTCTGGG |
| | | | AAGATACATGTCTGCTCTTTTTGCTGATGATTTAAATCAGTTAACAGGA |
| | | | TATCACACAGATTTTTCTTCTGAAATCATTGGATACCAGTTGATGTGTC |
| | | | AACCAATTTTGTTGGGTGAAGCAGAATTGGCAAAAAACGTGTCTTTGA |
| | | | TTCTTGGAACAGTTTCTTGGAATCTTAAAAAGAGATACTTATTATCTGC |
| | | | TGGAATTTTTGGAGCTATCACAGATGTGTTTTACAAAGAAAACTCATA |
| | | | CAAAGTGCCAACAGACAATTACATAACAACATACGCCAGAATGGCTGC |
| | | | TCCAAAAGAAATCATCTTCCTTGAAGGAGAAACACTTTTTGGAGATGA |
| | | | TACAGTGATTGAAGTGGCAATTATTCTGGCATCTTTTTCTGCATCCACA |
| | | | AGAAGAAGGGGAAAACTGCTGGAACAGTGGAATCTTGTGATTGGATTT |
| | | | AATAGAAATAGATTTCTTTACATTATTAAATTGATTTTTCTTTGGCTTCT |
| | | | TTGGCCTGTGACACTTGCTTGCTTTGTGCTTGCTGCTGTGTACTCTGAA |
| | | | CTGGTGATTGGAGCTGTGATTCTGAGAGGACACCTGAGAATTGCTGGA |
| | | | CACCACCTGGGAAGAACAGTGGCAACATCAAGAACACTTTCTTACTAC |
| | | | AAACTGGGAGCTTCTCAGAGAGTGAAAAGATACTCTGGCCTGATGTGG |
| | | | CTGAGCTACTTTGCTAGATATGCTGGTGGTTCTCTTGGCGGAGGGGGTT |
| | | | CGGGAATCGTGGGAATTGTGGCAGGACTGGCAGTGCTGGCCGTGGTGG |
| | | | TGATCGGAGCCGTGGTGGCTACCGTGATGTGCAGACGGAAGTCCAGCG |
| | | | GAGGCAAGGGCGGCAGCTACAGCCAGGCCGCCAGCTCTGATAGCGCC |
| | | | CAGGGCAGCGACGTGTCACTGACAGCCTAGTAA (SEQ ID NO: RS |
| | | | C8n3) |

TABLE 16
Highly predicted epitopes within the string sequences of RS C5 - RS C8 strings.

| Peptide (1) | Alleles (1) | Peptide (2) | Alleles (2) |
|---|---|---|---|
| AADLDDFS | HLA-A01:01 | LLNVPLHGTI | HLA-B51:01 |
| AADLDDFSKQ | HLA-A01:01 | LLNVPLHGTIL | HLA-A02:07 |
| AAEASKKPRQK | HLA-C02:02 | LLPAADLDDF | HLA-B13:01 |
| AGDSGFAA | HLA-C08:02 | LLQFAYANRNR | HLA-A31:01; HLA-A74:01 |
| AGDSGFAAYS | HLA-B46:01 | LLWPVTLACFV | HLA-A02:01; HLA-A02:04; HLA-A02:07; HLA-A02:11 |
| AGLPYGANKDG | HLA-C07:01 | LLWPVTLACFVL | HLA-B48:01 |
| AGNGGDAALAL | HLA-C01:02; HLA-C03:02; HLA- | LMWLSYFIASF | HLA-B35:01 |
| | C07:04; HLA-C12:03 | | |
| AIILASFSAS | HLA-C17:01 | LNVPLHGTI | HLA-B14:02 |
| AIKLDDKD | HLA-E01:01 | LNVPLHGTIL | HLA-C15:02; HLA-G01:01; HLA-G01:04 |
| AIKLDDKDPNF | HLA-E01:01 | LPKEITVATS | HLA-B54:01; HLA-B55:01; HLA-B55:02; HLA-B56:01 |
| AIVLQLPQGTTL | HLA-B15:09 | LPKEITVATSR | HLA-A33:03; HLA-A34:01 |
| ALNTPKDHI | HLA-A02:02; HLA-B13:01; HLA-B13:02 | LPQGTTLP | HLA-A25:01 |
| AMACLVGLMW | HLA-A32:01 | LPQGTTLPK | HLA-A30:01; HLA-A34:02 |
| APKEIIFLEGET | HLA-B81:01 | LPQGTTLPKG | HLA-B55:01 |
| APSASAFFG | HLA-B35:02 | LPQRQKKQQTV | HLA-B07:04; HLA-B42:01 |
| ARMAGNGGDA | HLA-C07:01 | LPYGANKDGII | HLA-B51:01; HLA-B55:01; HLA-B81:01; HLA-C02:02 |
| ARMAGNGGDAA | HLA-B39:06 | LPYGANKDGIIW | HLA-B35:01; HLA-B35:07; HLA-B51:01; HLA-B53:01 |
| ARMAGNGGDAAL | HLA-B15:09; HLA-B27:05 | LQLPQGTTLPK | HLA-A03:01; HLA-A11:01; HLA-A11:02 |
| ASAFFGMSRI | HLA-B49:01 | LQQSMSSA | HLA-B39:06 |
| ASKKPRQKRTA | HLA-E01:01 | LSYFIASFRL | HLA-A03:01; HLA-A68:02; HLA-B15:17; HLA- |
| | | | B57:01; HLA-B58:02; HLA-C15:02 |
| ASKKPRQKRTAT | HLA-E01:01 | LSYYKLGASQR | HLA-A31:01 |
| ASQRVAGDSGF | HLA-B57:03 | LTRPLLESEL | HLA-B57:01; HLA-B57:03 |
| ATKAYNVTQAF | HLA-B57:01; HLA-B57:03; HLA-B58:02 | LTYTGAIK | HLA-A03:01; HLA-A34:02 |
| ATSRTLSYYKL | HLA-B57:03 | LWPVTLACFV | HLA-C04:01 |
| AVILRGHLRIA | HLA-B08:01 | LWPVTLACFVL | HLA-A23:01; HLA-A24:07; HLA-C04:01 |
| AVYRINWIT | HLA-B48:01 | MAGNGGDAALAL | HLA-B35:02 |
| AYKTFPPTEP | HLA-C18:01 | MEVTPSGTWLT | HLA-B40:06 |
| AYKTFPPTEPK | HLA-A31:01; HLA-A33:03 | MWLSYFIASFR | HLA-A31:01; HLA-A33:03 |
| AYKTFPPTEPKK | HLA-A30:01 | MWSFNPETNI | HLA-A24:02 |
| AYNVTQAFGRR | HLA-A31:01 | MWSFNPETNIL | HLA-A24:02 |
| AYSRYRIGNYK | HLA-A30:01 | MWSFNPETNILL | HLA-A24:02 |
| DAYKTFPPTE | HLA-B51:01 | NFKDQVILLN | HLA-A24:07 |
| DDFSKQLQQSM | HLA-A33:01 | NFKDQVILLNK | HLA-A30:01; HLA-A33:01; HLA-A33:03 |
| DDKDPNFKD | HLA-B35:02 | NLVIGFLFLTW | HLA-B44:02; HLA-B44:03 |
| DDKDPNFKDQV | HLA-A33:01 | NPANNAAIVLQL | HLA-B35:01 |
| DDKDPNFKDQVI | HLA-B14:01 | NPETNILLNVPL | HLA-B81:01 |
| DHIGTRNP | HLA-B39:06 | NRFLYIIKLIFL | HLA-B27:05; HLA-C18:01 |
| DHSSSSDNIA | HLA-B39:06 | NSSRNSTPG | HLA-B58:01 |
| DIKDLPKEITVA | HLA-A26:01 | NTDHSSSSDNI | HLA-A01:01 |
| DKDPNFKDQVI | HLA-B38:02 | NVPLHGTI | HLA-B14:02; HLA-B51:01; HLA-C01:02 |
| DKKKKADE | HLA-E01:01 | NWITGGIAIA | HLA-B39:06 |
| DLPKEITVATSR | HLA-A68:01 | PARMAGNGGD | HLA-E01:01 |
| DLSPRWYF | HLA-B50:01 | PETNILLNVPL | HLA-C06:02 |
| DLSPRWYFYYL | HLA-A02:04; HLA-A33:01 | PHVGEIPV | HLA-B15:09 |
| DPSFLGRYMSA | HLA-B54:01 | PKDHIGTR | HLA-A33:01 |
| DSGFAAYSRY | HLA-A01:01; HLA-A25:01; HLA-A26:01 | PKEIIFLE | HLA-A33:01; HLA-B57:03 |
| DSGFAAYSRYR | HLA-A33:01; HLA-A66:01; HLA-A68:01 | PKKDKKKKA | HLA-C01:02 |
| DSNGTITVEEL | HLA-C04:03 | PKKDKKKKADE | HLA-C07:01 |
| DYKHWPQI | HLA-A23:01 | PLLESELVIG | HLA-A26:01 |
| DYKHWPQIA | HLA-A33:01 | PPTEPKKD | HLA-B14:01 |
| DYKHWPQIAQ | HLA-A33:03 | PPTEPKKDK | HLA-B81:01 |
| EAGLPYGAN | HLA-A34:01 | PQGTTLPK | HLA-A11:02 |
| EIPVAYRKVLL | HLA-B14:02; HLA-G01:01; HLA- | PQIAQFAPSASA | HLA-B39:06 |
| | G01:03; HLA-G01:04 | | |
| EITVATSRTLSY | HLA-A36:01 | PSFLGRYMSAL | HLA-C03:02 |
| ELIRQGTDYK | HLA-A34:01 | PTEPKKDK | HLA-A11:02 |
| EPKKDKKKKADE | HLA-E01:01 | PTEPKKDKK | HLA-B49:01 |
| EQWNLVIGFLF | HLA-A24:02; HLA-B27:05 | PVAYRKVL | HLA-B07:04; HLA-B42:01; HLA-B46:01 |
| EQWNLVIGFLFL | HLA-A68:02 | PYGANKDGIIW | HLA-A24:02; HLA-A24:07 |
| ESELVIGAVIL | HLA-A01:01 | QASSRSSSRSR | HLA-A33:01 |
| ESELVIGAVILR | HLA-A68:01 | QGNFGDQELI | HLA-B44:02 |
| ETNILLNVPLH | HLA-A26:01 | QGTTLPKGF | HLA-B44:02 |
| ETQALPQRQKK | HLA-A33:03; HLA-A34:02 | QLMCQPIL | HLA-B08:01 |
| EVGPEHSLAE | HLA-C07:04 | QQGQTVTKK | HLA-A11:02; HLA-A30:02 |
| EVTPSGTWLT | HLA-A68:02 | QQQGQTVTKK | HLA-A11:02 |
| FAAYSRYRIGNY | HLA-A26:01 | QQQQGQTVTKK | HLA-A11:02 |
| FAPSASAFFGM | HLA-A02:07; HLA-C02:02; HLA- | QRQKKQQTVTL | HLA-C18:01 |
| | C03:03; HLA-C03:04; HLA-C16:01 | | |
| FARTRSMW | HLA-B53:01 | QRVAGDSGFAAY | HLA-B27:05 |
| FDESGEFK | HLA-A11:02 | QTVTLLPAA | HLA-A02:05; HLA-A02:06 |
| FGMSRIGM | HLA-C12:03; HLA-C16:01 | QVILLNKHIDAY | HLA-A26:01 |
| FLWLLWPVTLA | HLA-A02:01; HLA-A02:04; HLA- | RCDIKDLPK | HLA-A36:01 |
| | A02:07; HLA-A02:11; HLA-B54:01 | | |
| FLYIIKLIFL WL | HLA-C17:01 | RFLYIIKLIFL W | HLA-A24:07; HLA-B44:03; HLA-B57:01 |
| FPPTEPKKDKKK | HLA-C07:01 | RGHLRIAGHHL | HLA-B48:01 |
| FRLFARTRSM | HLA-B14:01 | RGPEQTQGNF | HLA-B37:01 |
| FVLAAVYRINW | HLA-B57:01; HLA-B57:03 | RIGMEVTPSGTW | HLA-A32:01 |
| FYYLGTGPEAG | HLA-C14:02 | RINWITGGIA | HLA-B54:01 |
| FYYLGTGPEAGL | HLA-C03:03; HLA-C07:02; HLA- | RINWITGGIAI | HLA-B13:02; HLA-B27:05; HLA-B44:02; HLA-B55:02 |
| | C14:02; HLA-C14:03; HLA-C18:01 | | |
| GANKDGIIWVA | HLA-B40:06; HLA-B54:01 | RINWITGGIAIA | HLA-B55:02; HLA-B56:01 |
| GDAALALLLL | HLA-B40:02 | RLFARTRSMWSF | HLA-A32:01; HLA-B37:01; HLA-B48:01 |
| GDSGFAAY | HLA-A36:01 | RMAGNGGDAA | HLA-A02:02 |
| GFLFLTWICL | HLA-G01:04 | RMAGNGGDAAL | HLA-A02:02; HLA-A32:01; HLA-B13:01; HLA- |
| | | | B15:01; HLA-B48:01 |
| GGDAALALLLL | HLA-B14:01 | RNPANNAAIVL | HLA-A03:01; HLA-B07:04; HLA-G01:01; HLA- |
| | | | G01:03; HLA-G01:04 |
| GHHLGRCD | HLA-B15:10 | RNRFLYIIKLI | HLA-B14:01 |
| GIAIAMACL | HLA-G01:03 | RNRFLYIIKLIF | HLA-B14:01 |
| GLPYGANK | HLA-A03:01; HLA-A11:02 | RPLLESELVI | HLA-B55:01; HLA-B81:01 |
| GLPYGANKDGI | HLA-B13:01 | RPLLESELVIGA | HLA-B55:01; HLA-B55:02 |
| GNGGDAALAL | HLA-G01:01 | RQKKQQTVTLL | HLA-B13:02; HLA-B15:01; HLA-B48:01; HLA-B52:01 |
| GNGGDAALALLL | HLA-G01:04 | RQKRTATKAYNV | HLA-B13:02 |
| GRCDIKDLPKE | HLA-B14:01 | RSMWSFNPETNI | HLA-B58:02 |
| GSRGGSQASSR | HLA-A31:01 | RSSSRSRNSSR | HLA-A31:01 |
| GSSRGTSPAR | HLA-A68:02 | SAAEASKKPRQK | HLA-A11:02; HLA-B15:02 |
| GTILTRPLLE | HLA-A68:02 | SAFFGMSRIGM | HLA-C12:03 |
| GTITVEELKKL | HLA-B57:01 | SAFFGMSRIGME | HLA-C12:03 |
| GTRNPANNAA | HLA-A30:01 | SELVIGAVILR | HLA-A34:01; HLA-B44:02 |
| HGTILTRP | HLA-A02:02; HLA-A02:07; HLA-B35:03 | SEVGPEHSLAE | HLA-C07:04 |
| HGTILTRPL | HLA-B15:09; HLA-B15:10; HLA-B35:03 | SMWSFNPET | HLA-A02:01; HLA-A02:04; HLA-A02:11 |
| HGTILTRPLL | HLA-B14:02; HLA-B15:09; HLA-G01:04 | SMWSFNPETNI | HLA-A02:01; HLA-A02:04; HLA-A02:11 |
| HHLGRCDI | HLA-B38:01; HLA-B38:02 | SSRGTSPARM | HLA-A30:01; HLA-C02:02 |
| HIDAYKTFPP | HLA-B41:01 | SSRNSTPGS | HLA-A30:01 |
| HLRIAGHHLGR | HLA-A33:03 | SSRNSTPGSSR | HLA-A33:03; HLA-A66:01 |
| HSSSSDNI | HLA-B38:02 | SYFIASFRLFAR | HLA-A31:01; HLA-A33:01 |
| HSSSSDNIALL | HLA-A68:02; HLA-B38:01; HLA- | TATKAYNVTQA | HLA-B54:01 |
| | B38:02; HLA-B57:03; HLA-C05:01 | | |
| HTTDPSFLGRYM | HLA-B57:01 | TDHSSSSDNIAL | HLA-B35:02 |
| HVGEIPVAYRK | HLA-A03:01; HLA-A11:01; HLA- | TDPSFLGR | HLA-A34:02; HLA-A36:01; HLA-A66:01 |
| | A11:02; HLA-A31:01; HLA-A34:02; HLA- | | |
| | A68:01 | | |
| HVGEIPVAYRKV | HLA-A68:02 | TDPSFLGRYM | HLA-A36:01 |
| IAQFAPSASA | HLA-B54:01; HLA-B55:02; HLA-B56:01 | TDVFYKENSY | HLA-B18:01 |
| IAQFAPSASAF | HLA-B46:01; HLA-C02:02; HLA- | TEGALNTPK | HLA-A11:02; HLA-A30:02; HLA-A36:01 |
| | C03:02; HLA-C03:03; HLA-C03:04; HLA- | | |
| | C07:04; HLA-C12:02; HLA-C14:02; HLA- | | |
| | C16:01 | | |
| IASFRLFA | HLA-C16:01 | TEPKKDKKK | HLA-A34:02 |
| IGMEVTPSGTW | HLA-A24:02; HLA-A32:01; HLA- | TGAIKLDD | HLA-B14:01 |
| | B44:02; HLA-B53:01; HLA-B57:01; HLA- | | |
| | B58:01 | | |
| IIKLIFLWLL | HLA-A33:01 | TGGIAIAM | HLA-C07:04 |
| IIKLIFLWLLW | HLA-B44:02; HLA-B57:01 | TGPEAGLPYGA | HLA-A02:07 |
| IIWVATEGA | HLA-A02:11 | TILTRPLL | HLA-A30:01; HLA-B08:01; HLA-B14:02; HLA- |
| | | | B50:01; HLA-C03:02; HLA-C06:02; HLA-G01:01; HLA- |
| | | | G01:03; HLA-G01:04 |
| IIWVATEGAL | HLA-C08:01 | TILTRPLLE | HLA-C03:02 |
| IKDLPKEITV | HLA-B18:01 | TIQPRVEK | HLA-A23:01; HLA-A30:01 |
| IKDLPKEITVA | HLA-C04:03 | TITVEELKKLLE | HLA-B14:01 |
| ILLNVPLHGTI | HLA-A02:07; HLA-B51:01 | TKAYNVTQAF | HLA-B15:03 |
| ILTRPLLESEL | HLA-A02:02; HLA-B27:05 | TLACFVLAAVYR | HLA-A68:01 |
| ITGGIAIAMA | HLA-A02:05; HLA-A68:02 | TLLPAADLDDF | HLA-B38:02 |
| ITVATSRTLSYY | HLA-A36:01 | TLPKGFYA | HLA-A02:11 |
| ITVEELKKLLE | HLA-B08:01 | TLSYYKLGA | HLA-A02:03 |
| IVLQLPQGTTL | HLA-A02:11; HLA-B38:01 | TNILLNVPL | HLA-B15:09; HLA-G01:03 |
| KAYNVTQAFGRR | HLA-A03:01; HLA-A31:01 | TPGSSRGTSPA | HLA-B07:02; HLA-B55:01; HLA-B55:02; HLA-B56:01 |
| KDGIIWVAT | HLA-B37:01 | TPKDHIGTRNP | HLA-B55:01; HLA-B55:02 |
| KDHIGTRNP | HLA-B41:01 | TQALPQRQKK | HLA-A03:01; HLA-A11:02 |
| KDLPKEITVA | HLA-B40:06 | TRNPANNA | HLA-B39:06 |
| KDLSPRWYFYY | HLA-A30:02 | TRNPANNAAIVL | HLA-B15:09; HLA-B38:01 |
| KEIIFLEGET | HLA-B40:06; HLA-B49:01 | TSRTLSYYKL | HLA-B58:02; HLA-G01:04 |
| KEITVATSRT | HLA-B40:02; HLA-E01:01 | TSRTLSYYKLG | HLA-A02:01 |
| KHWPQIAQFA | HLA-B39:06 | TTDPSFLGRYMS | HLA-A01:01 |
| KHWPQIAQFAP | HLA-B39:06 | TTLPKGFYA | HLA-A02:06 |
| KKDKKKKAD | HLA-E01:01 | TVATSRTLS | HLA-B46:01 |
| KKKADETQA | HLA-B15:03 | VATEGALNTPK | HLA-A11:01; HLA-A34:02 |
| KKKKADETQAL | HLA-B48:01 | VEELKKLLEQW | HLA-B44:02 |
| KLDDKDPNFKD | HLA-A02:07 | VGEIPVAY | HLA-A33:01; HLA-A36:01; HLA-B15:03; HLA-B58:02 |
| KLIFLWLLWPV | HLA-A02:04; HLA-A02:11 | VGEIPVAYRK | HLA-A11:01 |
| KPRQKRTATKA | HLA-B07:04; HLA-B55:01; HLA- | VGLMWLSY | HLA-B46:01 |
| | B55:02; HLA-B56:01 | | |
| KSAAEASKKPR | HLA-A74:01 | VIGAVILRGHL | HLA-C04:03 |
| KTFPPTEPKKD | HLA-B58:02 | VILLNKHID | HLA-E01:01 |
| KTFPPTEPKKDK | HLA-A11:01; HLA-A68:02 | VLAAVYRINW | HLA-A32:01; HLA-B57:01 |
| KVPTDNYI | HLA-B38:02 | VPLHGTILTR | HLA-A33:01 |
| KVPTDNYIT | HLA-G01:01 | VTKKSAAEA | HLA-A02:05 |
| LDDKDPNF | HLA-B37:01 | VTLLPAADLD | HLA-A68:02 |
| LDDKDPNFK | HLA-A11:02 | VTQAFGRR | HLA-A66:01 |
| LDRLNQLE | HLA-B14:01 | WPVTLACF | HLA-B35:07 |
| LESELVIGAV | HLA-B08:01; HLA-B49:01 | WSFNPETNILL | HLA-B35:01; HLA-B50:01 |
| LESELVIGAVIL | HLA-B40:01; HLA-B44:02; HLA-B44:03 | YAEGSRGGSQA | HLA-A26:01 |
| LFARTRSMW | HLA-A24:07; HLA-A32:01; HLA-B58:01 | YANRNRFLYII | HLA-B51:01 |
| LFARTRSMWSF | HLA-A24:07 | YFYYLGTGPEA | HLA-B54:01; HLA-C14:02; HLA-C14:03 |
| LGTGPEAGL | HLA-B15:09 | YGANKDGII | HLA-C02:02 |
| LHGTILTRP | HLA-B15:09; HLA-B15:10 | YGANKDGIIW | HLA-B53:01; HLA-B57:01; HLA-B58:01 |
| LHGTILTRPL | HLA-B15:09; HLA-B15:10 | YIIKLIFLWLL | HLA-A02:01; HLA-A02:02; HLA-A02:04; HLA- |
| | | | A02:07; HLA-A02:11; HLA-A29:02; HLA-B13:02 |
| LHGTILTRPLL | HLA-B15:09 | YKLGASQRV | HLA-B15:03 |
| LIFLWLLW | HLA-B57:01 | YLGTGPEA | HLA-A02:01; HLA-B55:01; HLA-C03:03 |
| LIFLWLLWPV | HLA-A02:04 | YLGTGPEAG | HLA-A02:03 |
| LIRQGTDYK | HLA-A30:01 | YLGTGPEAGLPY | HLA-A01:01; HLA-A29:02; HLA-B46:01 |
| LIRQGTDYKHW | HLA-A25:01; HLA-B57:01 | YRINWITGGI | HLA-B27:05 |
| LLESELVIGAV | HLA-A02:07 | YRINWITGGIAI | HLA-B27:05 |
| LLNKHIDAYKT | HLA-B58:02 | YSRYRIGNYK | HLA-A30:01 |
| LLNKHIDAYKTF | HLA-B48:01 | YYKLGASQRV | HLA-C18:01 |

1-61. (canceled)

62. A method of treating or preventing an infection by a virus or treating a respiratory disease or condition associated with an infection by a virus comprising administering to a subject in need thereof a pharmaceutical composition comprising: a polypeptide having an amino acid sequence with at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15; or a recombinant polynucleotide encoding a polypeptide having an amino acid sequence with at least 70%, at least 75%, at least 80%, at least 85%, at least 90%, at least 95%, or 100% sequence identity to a sequence of any one of the sequences depicted in column 2 of Table 11, column 2 of Table 12 or column 3 of Table 15.

63. The method of claim 62, wherein the subject has an immunodeficiency.

64. The method of claim 63, wherein the subject has a B cell immunodeficiency.

65. The method of claim 62, wherein the pharmaceutical composition comprises a polypeptide with at least 70%, at least 75%, at least 80%, at least 85%, at least 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2 and RS C8p2full; or a polynucleotide encoding a polypeptide with at least 70%, at least 75%, at least 80%, at least 85%, at least 90%, at least 95%, or 100% sequence identity to a sequence selected from the group consisting of RS Clp1full, RS C2p1full, RS C3p1full, RS C4p1full, RS C5p1, RS C5p2, RS C5p2full, RS C6p1, RS C6p2, RS C6p2full, RS C7p1, RS C7p2, RS C7p2full, RS C7p4, RS C7p4full, RS C8p1, RS C8p2, and RS C8p2full.

66. The method of claim 62, wherein the pharmaceutical composition comprises a polynucleotide with at least 70%, at least 75%, at least 80%, at least 85%, at least 90%, at least 95%, or 100% sequence identity to a sequence selected from the group consisting of SEQ ID NOs: RS C1n1, RS C2n1, RS C3n1, RS C4n1, RS C5n1, RS Con1, RS C7n1, RS C8n1, RS C5n2, RS Con2, RS C7n2, RS C8n2, RS C5n2full, RS Con2full, RS C7n2full, RS C8n2full, RS C7n4, RS C7n4full.

67. The method of claim 62, wherein the polynucleotide is an mRNA.

68. The method of claim 62, wherein the pharmaceutical composition further comprises one or more lipids.

69. The method of claim 68, wherein the one or more lipids comprise a lipid nanoparticle (LNP), and wherein the LNP encapsulates the recombinant polynucleotide construct.

71-115. (canceled)

116. A pharmaceutical composition comprising:
(i) a first recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and
(ii) a second recombinant polynucleotide encoding a 2019 SARS-CoV 2 spike protein or a variant or fragment thereof.

117. The pharmaceutical composition of claim 116, wherein the pharmaceutical composition comprises a nanoparticle, wherein the nanoparticle comprises the first recombinant polynucleotide and the second recombinant polynucleotide.

118. The pharmaceutical composition of claim 117, wherein the nanoparticle is present in the pharmaceutical composition at a dose of from 100 ng to 500 micrograms.

119. The pharmaceutical composition of claim 116, wherein the mass ratio of the first recombinant polynucleotide to the second recombinant polynucleotide is from about 1:50 to 50:1.

120. A composition comprising:
(i) a first pharmaceutical composition comprising a recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and
(ii) a second pharmaceutical composition comprising a recombinant polynucleotide encoding a 2019 SARS-CoV 2 spike protein or a variant or fragment thereof.

121. The composition of claim 120, wherein the first pharmaceutical composition comprises a first nanoparticle, wherein the first nanoparticle comprises the recombinant polynucleotide encoding a polypeptide comprising at least two of the following (a) a sequence comprising an epitope sequence from ORF1ab, a sequence comprising an epitope sequence from membrane glycoprotein (M), and a sequence comprising an epitope sequence from nucleocapsid phosphoprotein (N); and wherein the second pharmaceutical composition comprises a second nanoparticle, wherein the second nanoparticle comprises the recombinant polynucleotide encoding a 2019 SARS-CoV 2 spike protein or a variant or fragment thereof.

122. The composition of claim 121, wherein the mass ratio of the recombinant polynucleotide in (i) to the recombinant polynucleotide in (ii) is from about 1:50 to 50:1.

123. The composition of claim 121, wherein the first nanoparticle is present in the first pharmaceutical composition at a dose of from about 100 ng to 500 micrograms.

124. The composition of claim 121, wherein the second nanoparticle is present in the second pharmaceutical composition at a dose of from about 100 ng to 500 micrograms.

125. The composition of claim 121, wherein the recombinant polynucleotide in (i) is present in the first pharmaceutical composition at a dose of from about 50 ng to 250 micrograms.

126. The composition of claim 121, wherein the recombinant polynucleotide in (ii) is present in the second pharmaceutical composition at a dose of from about 50 ng to 250 micrograms.

127. The composition of claim 121, wherein the nanoparticle is a lipid nanoparticle.

128-149. (canceled)
